(19) United States
(12) Patent Application Publication
(10) Pub. No.: US 2025/0333397 A1
(43) Pub. Date: Oct. 30, 2025

(54) COMPOUNDS AND METHODS FOR MODULATING SPLICING

(71) Applicant: REMIX THERAPEUTICS INC., Watertown, MA (US)

(72) Inventors: Dominic Reynolds, Stoneham, MA (US); Michael W. Seiler, Belmont, MA (US); Anant A. Agrawal, Waltham, MA (US); Frederic Vaillancourt, Newton, MA (US); Peter Smith, Arlington, MA (US); Sudeep Prajapati, Somerville, MA (US); Allen T. Hopper, Lexington, MA (US); Stepan Vyskocil, Benesov (CZ)

(21) Appl. No.: 18/688,100

(22) PCT Filed: Aug. 30, 2022
(86) PCT No.: PCT/US2022/075712

§ 371 Date: Feb. 29, 2024

Related U.S. Application Data
(60) Provisional application No. 63/238,697, filed on Aug. 30, 2021.
(60) Provisional application No. 63/238,405, filed on Aug. 30, 2021.
(60) Provisional application No. 63/283,145, filed on Nov. 24, 2021.
(60) Provisional application No. 63/325,511, filed on Mar. 30, 2022.
(60) Provisional application No. 63/325,503, filed on Mar. 30, 2022.

(51) Int. Cl. C07D 403/14; A61K 31/501; A61K 31/506; A61K 31/513; A61K 31/53; C07D 401/14; C07D 405/14; C07D 409/14; C07D 413/14; C07D 417/14

(52) U.S. Cl. CPC C07D 403/14; A61K 31/501; A61K 31/506; A61K 31/513; A61K 31/53; C07D 401/14; C07D 405/14; C07D 409/14; C07D 413/14; C07D 417/14

(57) ABSTRACT

The present disclosure features compounds and related compositions that, inter alia, modulate nucleic acid splicing, e.g., splicing of a pre-mRNA, as well as methods of use thereof.

### CLAIM OF PRIORITY

This application is a U.S. National Phase Application under 35 U.S.C. § 371 of International Application No. PCT/US2022/075712, filed Aug. 30, 2022, which claims priority to U.S. Application No. 63/238,697, filed on Aug. 30, 2021; U.S. Application No. 63/238,405, filed on Aug. 30, 2021; U.S. Application No. 63/283,145, filed on Nov. 24, 2021; U.S. Application No. 63/325,503, filed on Mar. 30, 2022; and U.S. Application No. 63/325,511, filed on Mar. 30, 2022. The disclosure of each of the foregoing applications is incorporated herein by reference in its entirety.

### BACKGROUND

Alternative splicing is a major source of protein diversity in higher eukaryotes and is frequently regulated in a tissue-specific or development stage-specific manner. Disease associated alternative splicing patterns in pre-mRNAs are often mapped to changes in splice site signals or sequence motifs and regulatory splicing factors (Faustino and Cooper (2003), Genes Dev 17(4):419-37). Current therapies to modulate RNA expression involve oligonucleotide targeting and gene therapy; however, each of these modalities exhibit unique challenges as currently presented. As such, there is a need for new technologies to modulate RNA expression, including the development of small molecule compounds that target splicing.

### SUMMARY

The present disclosure features compounds and related compositions that, inter alia, modulate nucleic acid splicing, e.g., splicing of a pre-mRNA, as well as methods of use thereof. In an embodiment, the compounds described herein are compounds of Formula (I), (II), (III), or (IV), (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a)) and pharmaceutically acceptable salts, solvates, hydrates, tautomers, or stereoisomers thereof. The present disclosure additionally provides methods of using the compounds of the invention (e.g., compounds of Formulas (I), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a), and pharmaceutically acceptable salts, solvates, hydrates, tautomers, stereoisomers thereof), and compositions thereof, e.g., to target, and in embodiments bind or form a complex with, a nucleic acid (e.g., a pre-mRNA or nucleic acid component of a small nuclear ribonucleoprotein (snRNP) or spliceosome), a protein (e.g., a protein component of an snRNP or spliceosome, e.g., a member of the splicing machinery, e.g., one or more of the U1, U2, U4, U5, U6, U11, U12, U4atac, U6atac snRNPs), or a combination thereof. In another aspect, the compounds described herein may be used to alter the composition or structure of a nucleic acid (e.g., a pre-mRNA or mRNA (e.g., a pre-mRNA and the mRNA which arises from the pre-mRNA), e.g., by increasing or decreasing splicing at a splice site. In some embodiments, increasing or decreasing splicing results in modulating the level of a gene product (e.g., an RNA or protein) produced.

In another aspect, the compounds described herein may be used for the prevention and/or treatment of a disease, disorder, or condition, e.g., a disease, disorder or condition associated with splicing, e.g., alternative splicing. In some embodiments, the compounds described herein (e.g., compounds of Formulas (I), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a), and pharmaceutically acceptable salts, solvates, hydrates, tautomers, stereoisomers thereof) and compositions thereof are used for the prevention and/or treatment of a proliferative disease, disorder, or condition (e.g., a disease, disorder, or condition characterized by unwanted cell proliferation, e.g., a cancer or a benign neoplasm) in a subject. In some embodiments, the compounds described herein (e.g., compounds of Formulas (I), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a), and pharmaceutically acceptable salts, solvates, hydrates, tautomers, stereoisomers thereof) and compositions thereof are used for the prevention and/or treatment of a non-proliferative disease, disorder, or condition. In some embodiments, the compounds described herein (e.g., compounds of Formulas (I), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a) and pharmaceutically acceptable salts, solvates, hydrates, tautomers, stereoisomers thereof) and compositions thereof are used for the prevention and/or treatment of a neurological disease or disorder, an autoimmune disease or disorder, immunodeficiency disease or disorder, a lysosomal storage disease or disorder, a cardiovascular disease or disorder, a metabolic disease or disorder, a respiratory disease or disorder, a renal disease or disorder, or an infectious disease in a subject.

In one aspect, the present disclosure provides compounds of Formula (I):

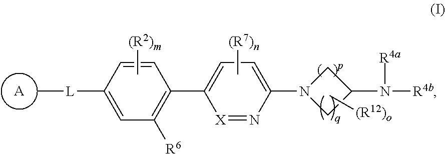

or a pharmaceutically acceptable salt, solvate, hydrate, tautomer, or stereoisomer thereof, wherein A is heteroaryl optionally substituted with one or more R¹; L is absent, —O—, —C(O)—, —N(R³)—; X is C(R⁵) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, C₁-C₆ alkylene-aryl, C₁-C₆ alkenylene-aryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² and R⁷ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D} NR^{B}C(O)R^{D}, or —C(O)NR^{B}R^{C}; each R³ is independently hydrogen, C₁-C₆-alkyl, C₁-C₆-haloalkyl, or cycloalkyl; each of R^{4a} and R^{4b} is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R⁹; or R^{4a} and R^{4b} are taken together with the nitrogen atom to which they are attached to form a heterocyclyl or heteroaryl, wherein the heterocyclyl and heteroaryl are optionally substituted with one or more R⁹; R⁵ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D} NR^{B}C(O)R^{D}, or —C(O)NR^{B}R^{C}; R⁶ is C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D}NR^{B}C(O)R^{D}, or —C(O)NR^{B}R^{C}; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NBC(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R⁹ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —NR^{B}R^{C}, —NR^{B}C(O)R^{D}—C(O)NR^{B}R^{C}, —C(O)R^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹⁰; each R¹⁰, R¹¹, and R¹²is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, halo, oxo, cyano, —OR^{A}, or —NR^{B}R^{C}; each R^{A} is independently hydrogen, C₁-C₆ alkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹³; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; R¹³ is C₁-C₆-alkyl or halo; m is 0, 1, 2, or 3; n is 0, 1, or 2; p and q are each independently 1, 2, 3, or 4; o is 0, 1, 2, 3, 4, 5, 6, 7, 8, 9, 10, 11, 12, or 13; and x is 0, 1, or 2.

In another aspect, the present disclosure features a compound of Formula (II):

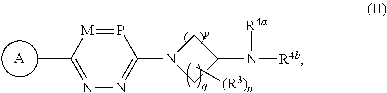

or a pharmaceutically acceptable salt, solvate, hydrate, tautomer, or stereoisomer thereof, wherein A is heteroaryl optionally substituted with one or more R¹; M and P are each independently C(R²) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NBC(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; q is 0, 1, 2, or 3; and x is 0, 1, or 2.

In another aspect, the present disclosure features a compound of Formula (III):

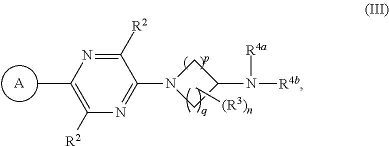

or a pharmaceutically acceptable salt, solvate, hydrate, tautomer, or stereoisomer thereof, wherein A is heteroaryl optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R6 are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; q is 0, 1, 2, or 3; and x is 0, 1, or 2.

In another aspect, the present disclosure features a compound of Formula (IV):

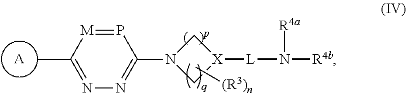

or a pharmaceutically acceptable salt, solvate, hydrate, tautomer, or stereoisomer thereof, wherein A is heteroaryl optionally substituted with one or more R¹; M and P are each independently C(R²) or N; X is C(R³) or N; L is absent, C₁-C₆-alkylene, C₂-C₆-alkenylene, C₁-C₆-heteroalkylene, —C(O)—, —NR^{B}C(O)—, —C(O)NR^{B}—; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R6 are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; q is 0, 1, 2, or 3; and x is 0, 1, or 2.

In another aspect, the present invention provides pharmaceutical compositions comprising a compound of Formula (I), (II), (III), (IV) (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a)), or a pharmaceutically acceptable salt, solvate, hydrate, tautomer, or stereoisomer thereof, and optionally a pharmaceutically acceptable excipient. In an embodiment, the pharmaceutical compositions described herein include an effective amount (e.g., a therapeutically effective amount) of a compound of Formula (I), (II), (III), (IV) (e.g., a compound of Formulas (I), (II), (III), (IV), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a) or a pharmaceutically acceptable salt, solvate, hydrate, tautomer, or stereoisomer thereof.

In another aspect, the present disclosure provides methods for modulating splicing, e.g., splicing of a nucleic acid (e.g., a DNA or RNA, e.g., a pre-mRNA) with a compound of Formula (I), (II), (III), (IV) (e.g., a compound of Formulas (I), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a) or a pharmaceutically acceptable salt, solvate, hydrate, tautomer, or stereoisomer thereof. In another aspect, the present disclosure provides compositions for use in modulating splicing, e.g., splicing of a nucleic acid (e.g., a DNA or RNA, e.g., a pre-mRNA) with a compound of Formula (I), (II), (III), (IV) (e.g., a compound of Formulas (I), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a) or a pharmaceutically acceptable salt, solvate, hydrate, tautomer, or stereoisomer thereof. Modulation of splicing may comprise impacting any step involved in splicing and may include an event upstream or downstream of a splicing event. For example, in some embodiments, the compound of Formula (I), (II), (III), (IV) binds to a target, e.g., a target nucleic acid (e.g., DNA or RNA, e.g., a precursor RNA, e.g., a pre-mRNA), a target protein, or combination thereof (e.g., an snRNP and a pre-mRNA). A target may include a splice site in a pre-mRNA or a component of the splicing machinery, such as the U1 snRNP. In some embodiments, the compound of Formula (I), (II), (III), (IV) alters a target nucleic acid (e.g., DNA or RNA, e.g., a precursor RNA, e.g., a pre-mRNA), target protein, or combination thereof. In some embodiments, the compound of Formula (I), (II), (III), (IV) increases or decreases splicing at a splice site on a target nucleic acid (e.g., an RNA, e.g., a precursor RNA, e.g., a pre-mRNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I), (II), (III), (IV), e.g., in a healthy or diseased cell or tissue). In some embodiments, the presence of a compound of Formula (I), (II), (III), (IV) results an increase or decrease of transcription of a target nucleic acid (e.g., an RNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I), (II), (III), (IV), e.g., in a healthy or diseased cell or tissue).

In another aspect, the present disclosure provides methods for preventing and/or treating a disease, disorder, or condition in a subject by administering a compound of Formula (I), (II), (III), (IV) (e.g., a compound of Formulas (I), (II), (III), (IV), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a)) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides methods of down-regulating the expression of (e.g., the level of or the rate of production of) a target protein with a compound of Formula (I), (II), (III), (IV) (e.g., a compound of Formulas (I), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a)) or a pharmaceutically acceptable salt, solvate, hydrate, tautomer, or stereoisomer thereof in a biological sample or subject. In another aspect, the present disclosure provides methods of up-regulating the expression of (e.g., the level of or the rate of production of) a target protein with a compound of Formula (I), (II), (III), (IV) (e.g., a compound of Formulas (I), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a)) or a pharmaceutically acceptable salt, solvate, hydrate, tautomer, or stereoisomer thereof in a biological sample or subject. In another aspect, the present disclosure provides methods of altering the isoform of a target protein with a compound of Formula (I), (II), (III), (IV) (e.g., a compound of Formulas (I), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a)) or a pharmaceutically acceptable salt, solvate, hydrate, tautomer, or stereoisomer thereof in a biological sample or subject. Another aspect of the disclosure relates to methods of inhibiting the activity of a target protein in a biological sample or subject. In some embodiments, administration of a compound of Formula (I), (II), (III), (IV) to a biological sample, a cell, or a subject comprises inhibition of cell growth or induction of cell death.

In another aspect, the present disclosure provides compositions for use in preventing and/or treating a disease, disorder, or condition in a subject by administering a compound of Formula (I), (II), (III), (IV) (e.g., a compound of Formulas (I), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a)) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides compositions for use in treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides compositions for use in down-regulating the expression of (e.g., the level of or the rate of production of) a target protein with a compound of Formula (I), (II), (III), (IV) (e.g., a compound of Formulas (I), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a)) or a pharmaceutically acceptable salt, solvate, hydrate, tautomer, or stereoisomer thereof in a biological sample or subject. In another aspect, the present disclosure provides compositions for use in up-regulating the expression of (e.g., the level of or the rate of production of) a target protein with a compound of Formula (I), (II), (III), (IV) (e.g., a compound of Formulas (I), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a)) or a pharmaceutically acceptable salt, solvate, hydrate, tautomer, or stereoisomer thereof in a biological sample or subject. In another aspect, the present disclosure provides compositions for use in altering the isoform of a target protein with a compound of Formula (I), (II), (III), (IV) (e.g., a compound of Formulas (I), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a)) or a pharmaceutically acceptable salt, solvate, hydrate, tautomer, or stereoisomer thereof in a biological sample or subject. Another aspect of the disclosure relates to compositions for use in inhibiting the activity of a target protein in a biological sample or subject. In some embodiments, administration of a compound of Formula (I), (II), (III), (IV) to a biological sample, a cell, or a subject comprises inhibition of cell growth or induction of cell death.

In another aspect, the present disclosure features kits comprising a container with a compound of Formula (I), (II), (III), (IV) (e.g., a compound of Formulas (I), (I-a), (I-b), (I-c), (I-d), (I-e), (I-f), (I-g), (I-h), (II-a), (II-b), (II-c), (II-d), (II-e), (II-f), (II-g), (II-h), (III-a), (III-b), (III-c), (III-d), (III-e), (III-f), (III-g), (IV-a)) or a pharmaceutically acceptable salt, solvate, hydrate, tautomer, stereoisomer thereof, or a pharmaceutical composition thereof. In certain embodiments, the kits described herein further include instructions for administering the compound of Formula (I), (II), (III), (IV) or the pharmaceutically acceptable salt, solvate, hydrate, tautomer, stereoisomer thereof, or the pharmaceutical composition thereof.

In any and all aspects of the present disclosure, in some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein other than a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, and WO 2019/199972. In some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, and WO 2019/199972, each of which is incorporated herein by reference in its entirety.

The details of one or more embodiments of the invention are set forth herein. Other features, objects, and advantages of the invention will be apparent from the Detailed Description, the Examples, and the Claims.

### DETAILED DESCRIPTION

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁o alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—CI₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized.

The heteroatom(s) 0, N, P, S, and Si may be placed at any position of the heteroalkyl group.

Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl. “Heterocyclyl” as used herein refers to a radical of a 3- to 16-membered non-aromatic ring system having ring carbon atoms and 1 to 12 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or more rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-16 membered heterocyclyl. In certain embodiments, the heterocyclyl group is substituted 3-16 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl.

Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substitutent having two oxygen atoms bound to a nitrogen atom, e.g., —NO₂.

As used herein, the term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, —O-alkyl, or other modification.

As used herein, the term “nucleic acid” refers to deoxyribonucleic acids (DNA) or ribonucleic acids (RNA) and polymers thereof in either single- or double-stranded form. The term “nucleic acid” includes a gene, cDNA, pre-mRNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein, “oxo” refers to a carbonyl, i.e., —C(O)—.

The symbol “ [symbol] ” as used herein in relation to a compound of Formula (I), (II), (III), (IV) refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The compounds provided herein may exist in one or more particular geometric, optical, enantiomeric, diasteriomeric, epimeric, stereoisomeric, tautomeric, conformational, or anomeric forms, including but not limited to: cis- and trans-forms; E- and Z-forms; endo- and exo-forms; R-, S-, and meso-forms; D- and L-forms; d- and 1-forms; (+) and (−) forms; keto-, enol-, and enolate-forms; syn- and anti-forms; synclinal- and anticlinal-forms; a- and p-forms; axial and equatorial forms; boat-, chair-, twist-, envelope-, and half chair-forms; and combinations thereof, hereinafter collectively referred to as “isomers” (or “isomeric forms”).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising an enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound.

In some embodiments, a diastereomerically pure compound can be present with other active or inactive ingredients. For example, a pharmaceutical composition comprising a diastereometerically pure exo compound can comprise, for example, about 90% excipient and about 10% diastereometerically pure exo compound. In certain embodiments, the diastereometerically pure exo compound in such compositions can, for example, comprise, at least about 95% by weight exo compound and at most about 5% by weight endo compound, by total weight of the compound. For example, a pharmaceutical composition comprising a diastereometerically pure endo compound can comprise, for example, about 90% excipient and about 10% diastereometerically pure endo compound. In certain embodiments, the diastereometerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; 0 may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal ofPharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I), (II), (III), (IV) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·xH₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomer” refers to compounds that are interchangeable forms of a particular compound structure, and that vary in the displacement of hydrogen atoms and electrons. Thus, two structures may be in equilibrium through the movement of R electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range. “Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a compound of Formula (I), (II), (III), (IV) refers to an amount sufficient to elicit the desired biological response, i.e., treating the condition. As will be appreciated by those of ordinary skill in this art, the effective amount of a compound of Formula (I), (II), (III), (IV) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I), (II), (III), (IV) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a compound described herein (e.g., a compound of Formula (I), (II), (III), (IV)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formula (I), (II), (III), (IV)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

A “non-proliferative disease” refers to a disease that does not primarily extend through the abnormal multiplication of cells. A non-proliferative disease may be associated with any cell type or tissue type in a subject. Exemplary non-proliferative diseases include neurological diseases or disorders (e.g., a repeat expansion disease); autoimmune disease or disorders; immunodeficiency diseases or disorders; lysosomal storage diseases or disorders; inflammatory diseases or disorders; cardiovascular conditions, diseases, or disorders; metabolic diseases or disorders; respiratory conditions, diseases, or disorders; renal diseases or disorders; and infectious diseases.

### Compounds

In one aspect, the present disclosure provides compounds of Formula (I):

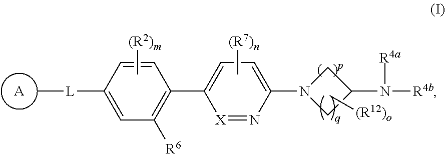

or a pharmaceutically acceptable salt, solvate, hydrate, tautomer, or stereoisomer thereof, wherein A is heteroaryl optionally substituted with one or more R¹; L is absent, —O—, —C(O)—, —N(R³)—; X is C(R⁵) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, C₁-C₆ alkylene-aryl, C₁-C₆ alkenylene-aryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² and R⁷ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D} NR^{B}C(O)R^{D}, or —C(O)NR^{B}R^{C}; each R³ is independently hydrogen, C₁-C₆-alkyl, C₁-C₆-haloalkyl, or cycloalkyl; each of R^{4a} and R^{4b} is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R⁹; or R^{4a} and R^{4b} are taken together with the nitrogen atom to which they are attached to form a heterocyclyl or heteroaryl, wherein the heterocyclyl and heteroaryl are optionally substituted with one or more R⁹; R⁵ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D} NR^{B}C(O)R^{D}, or —C(O)NR^{B}R^{C}; R⁶ is C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D}NR^{B}C(O)R^{D}, or —C(O)NR^{B}R^{C}; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NBC(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R⁹ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —NR^{B}R^{C}, —NR^{B}C(O)R^{D}—C(O)NR^{B}R^{C}, —C(O)R^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹⁰; each R¹⁰, R¹¹, and R¹² is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, halo, oxo, cyano, —OR^{A}, or —NR^{B}R^{C}; each R^{A} is independently hydrogen, C₁-C₆ alkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹³; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; R¹³ is C₁-C₆-alkyl or halo; m is 0, 1, 2, or 3; n is 0, 1, or 2; p and q are each independently 1, 2, 3, or 4; o is 0, 1, 2, 3, 4, 5, 6, 7, 8, 9, 10, 11, 12, or 13; and x is 0, 1, or 2.

As generally described herein, A is cycloalkyl, heterocyclyl, aryl, or heteroaryl, each of which is optionally substituted with one or more R¹. In some embodiments, A is independently a monocyclic ring, e.g., monocyclic cycloalkyl, monocyclic heterocyclyl, monocyclic aryl, or monocyclic heteroaryl. The monocyclic ring may be saturated, partially unsaturated, or fully unsaturated (e.g., aromatic). In some embodiments, A is a monocyclic ring comprising between 3 and 10 ring atoms (e.g., 3, 4, 5, 6, 7, 8, 9, or 10 ring atoms). In some embodiments, A is a 4-membered monocyclic ring. In some embodiments, A is a 5-membered monocyclic ring. In some embodiments, A is a 6-membered monocyclic ring. In some embodiments, A is a 7-membered monocyclic ring. In some embodiments, A is an 8-membered monocyclic ring. In some embodiments, A is independently a monocyclic ring optionally substituted with one or more R¹.

In some embodiments, A is a bicyclic heteroaryl optionally substituted with one or more R¹. The bicyclic ring may be saturated, partially unsaturated, or fully unsaturated (e.g., aromatic).

In some embodiments, A is a bicyclic ring comprising a fused, bridged, or spiro ring system. In some embodiments, A is independently a bicyclic ring comprising between 4 and 18 ring atoms (e.g., 4, 5, 6, 7, 8, 9, 10, 11, 12, 13, 14, 15, 16, 17, or 18 ring atoms). In some embodiments, A is a 6-membered bicyclic ring. In some embodiments, A is a 7-membered bicyclic ring. In some embodiments, A is an 8-membered bicyclic ring. In some embodiments, A is a 9-membered bicyclic ring. In some embodiments, A is a 10-membered bicyclic ring. In some embodiments, A is an 11-membered bicyclic ring. In some embodiments, A is a 12-membered bicyclic ring.

In some embodiments, A is a nitrogen-containing heteroaryl, e.g., heteroaryl comprising one or more nitrogen atom. The one or more nitrogen atom of the nitrogen-containing heteroaryl may be at any position of the ring. In some embodiments, A is a heteroaryl comprising at least 1, at least 2, at least 3, at least 4, at least 5, or at least 6 nitrogen atoms. In some embodiments, A is heteroaryl comprising 1 nitrogen atom. In some embodiments, A is heteroaryl comprising 2 nitrogen atoms. In some embodiments, A is heteroaryl comprising 3 nitrogen atoms. In some embodiments, A is heteroaryl comprising 4 nitrogen atoms. In some embodiments, A is a nitrogen-containing heteroaryl comprising one or more additional heteroatoms, e.g., one or more of oxygen, sulfur, boron, silicon, or phosphorus. In some embodiments, the one or more nitrogen of the nitrogen-containing heteroaryl is substituted, e.g., with R¹.

In some embodiments, A is selected from:

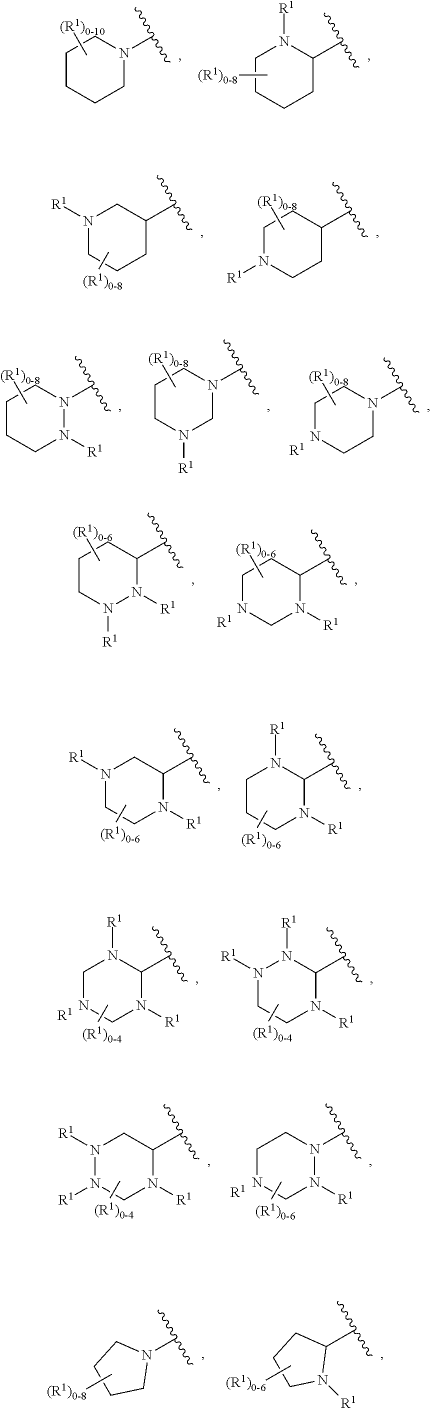

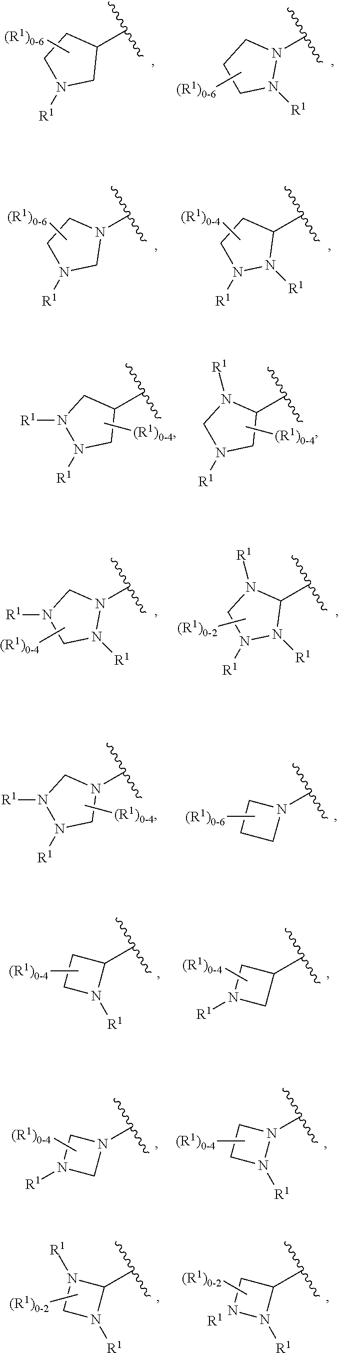

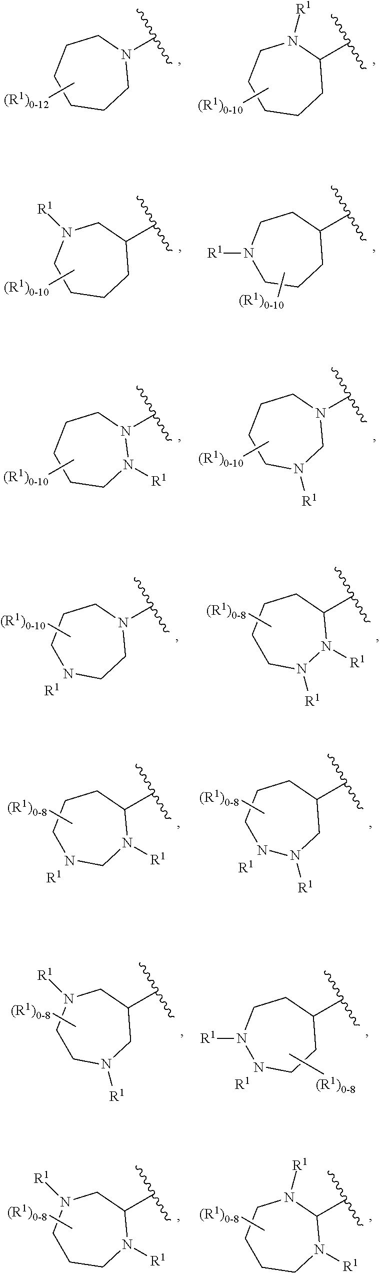

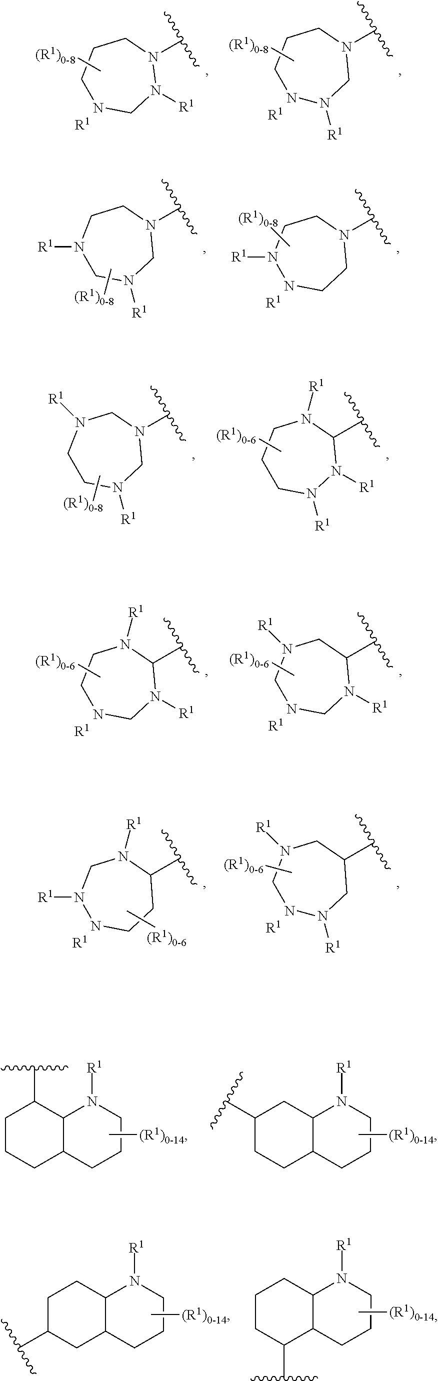

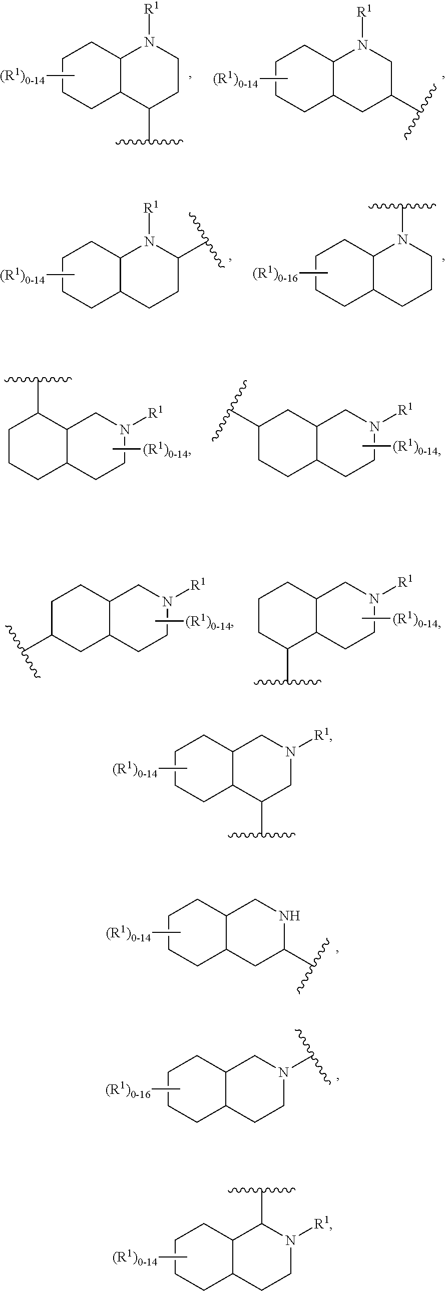

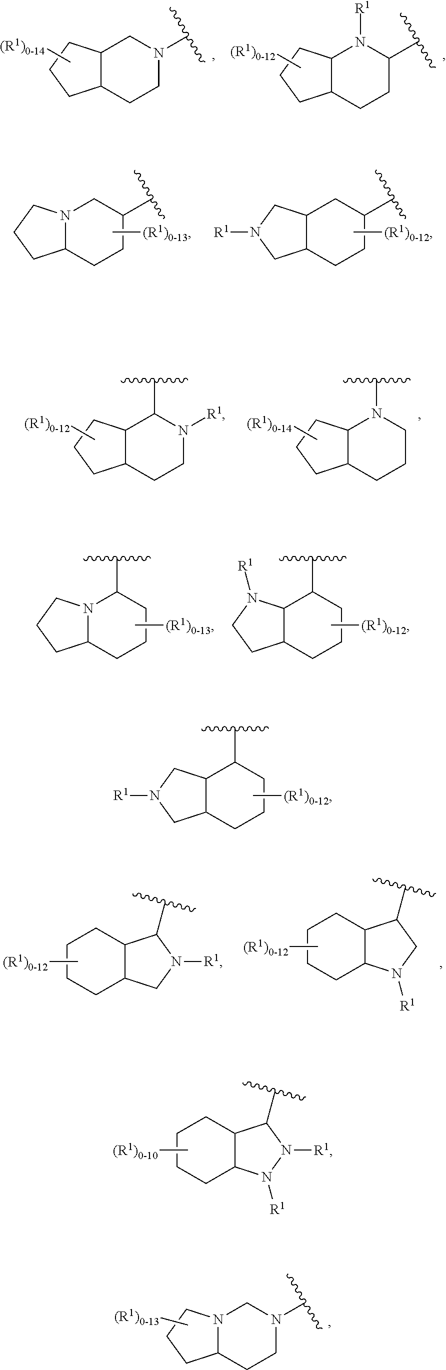

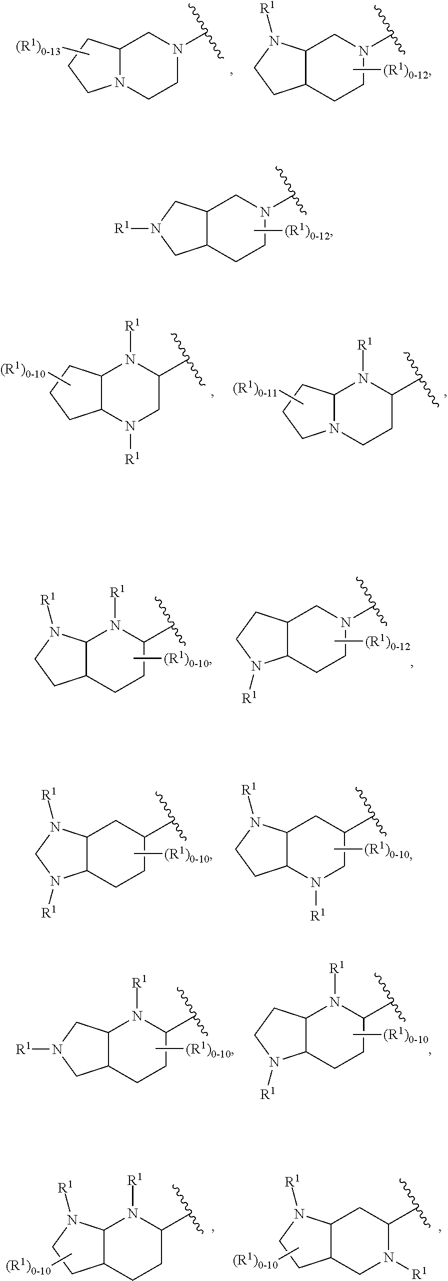

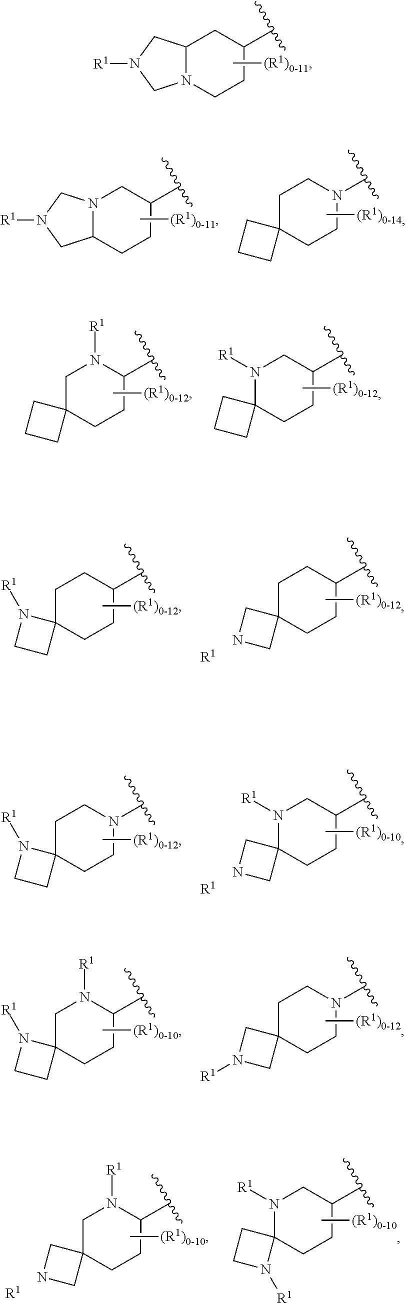

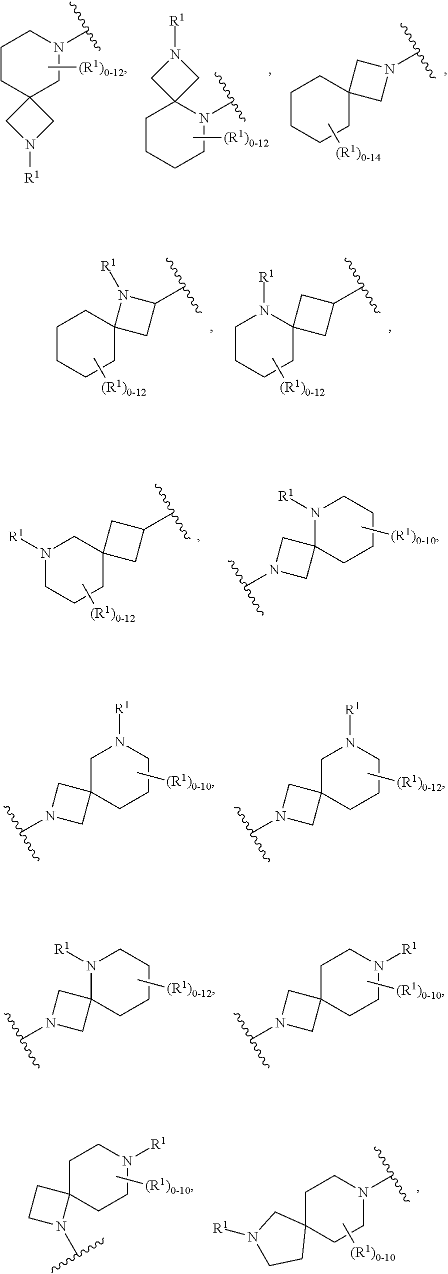

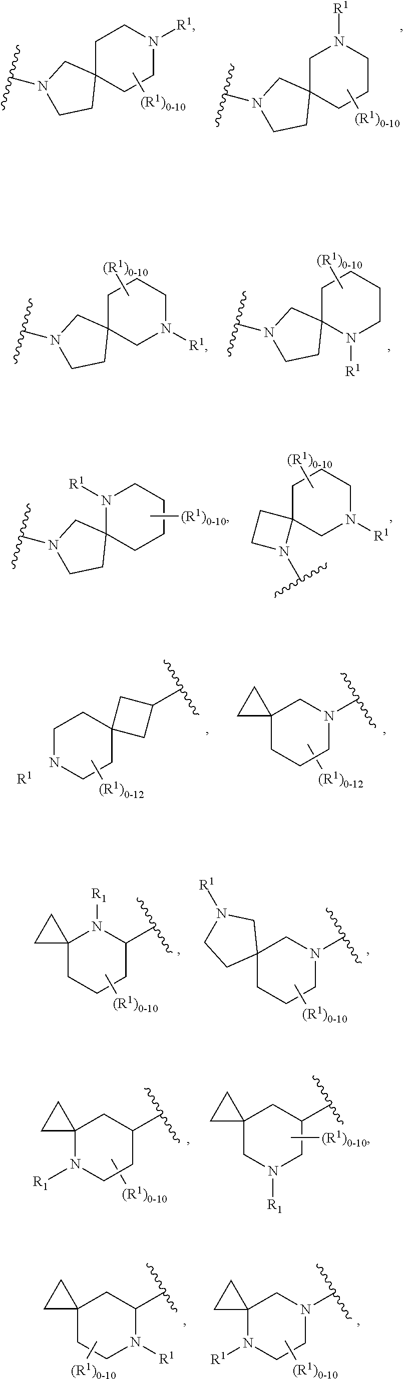

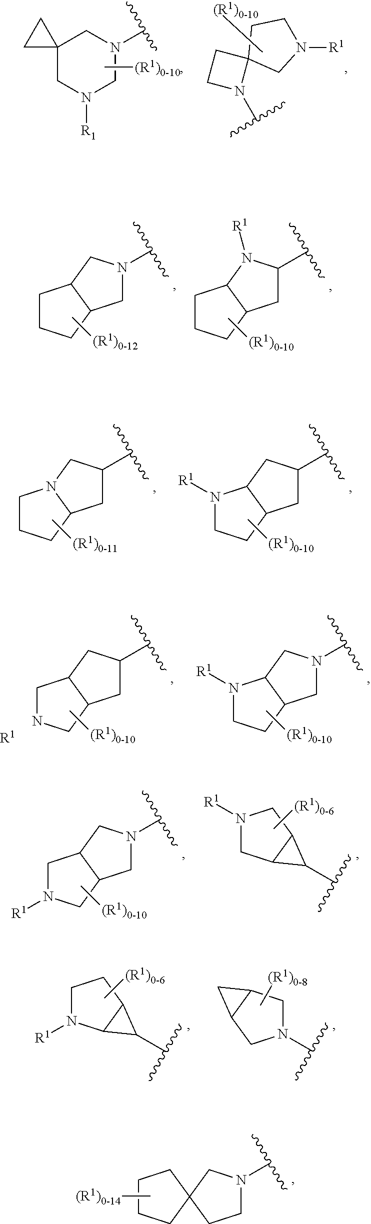

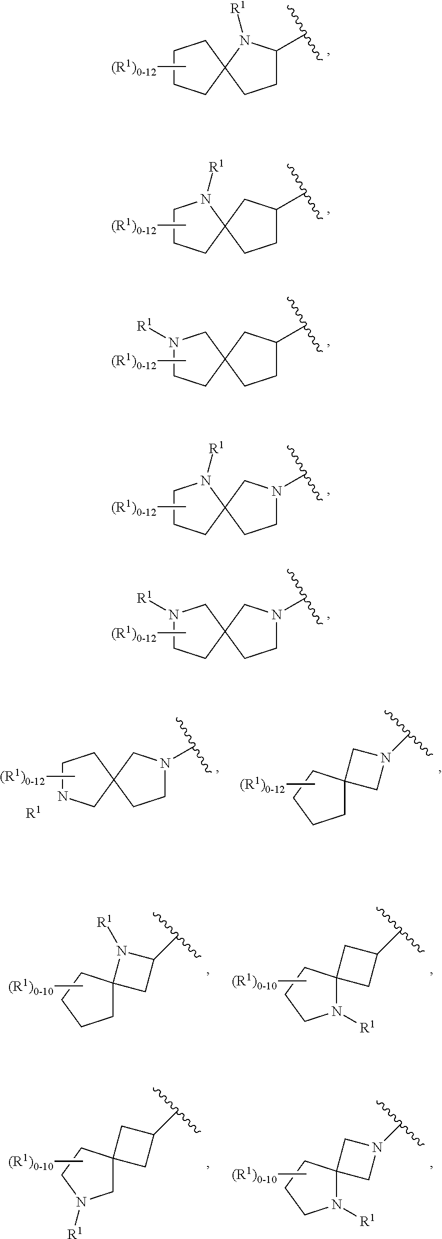

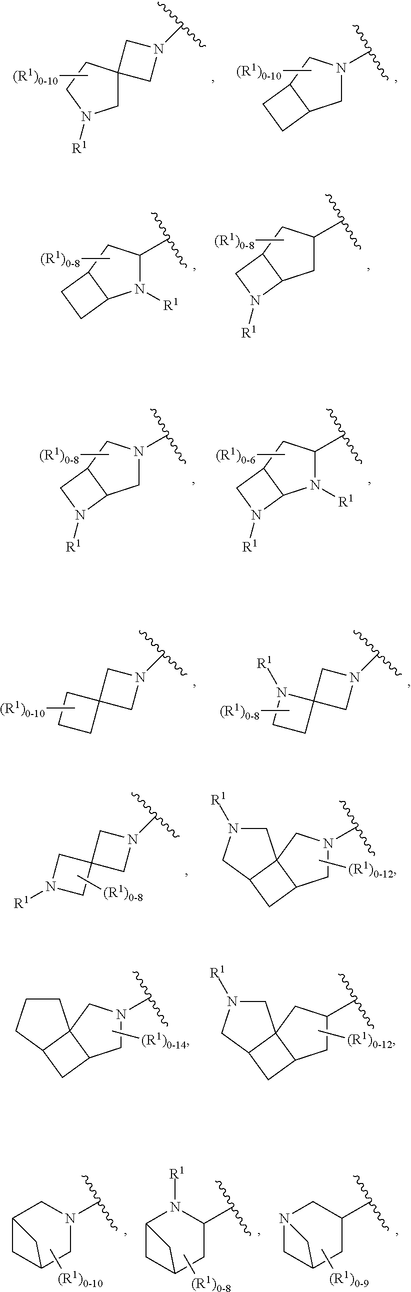

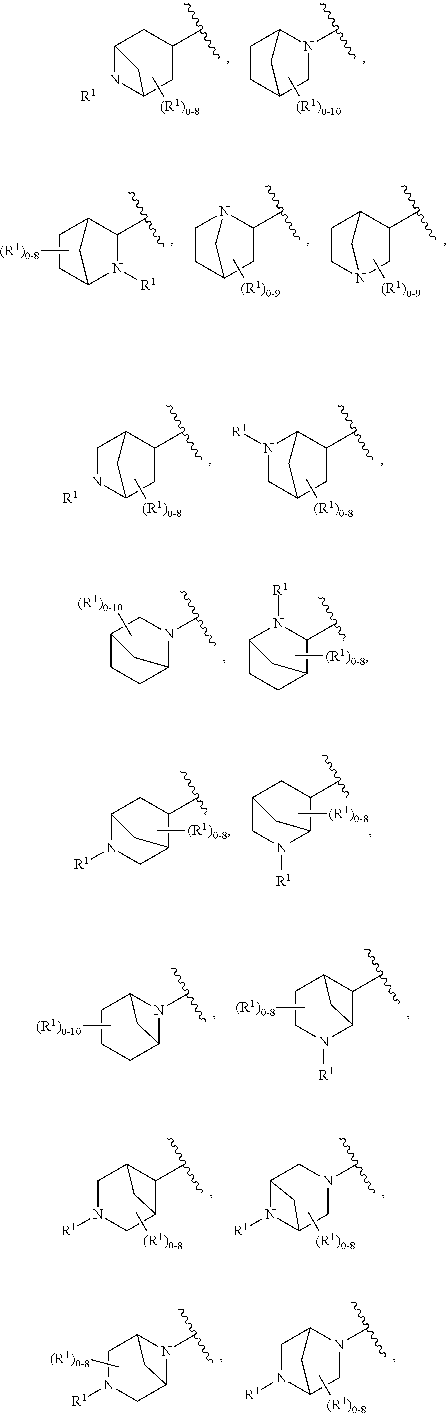

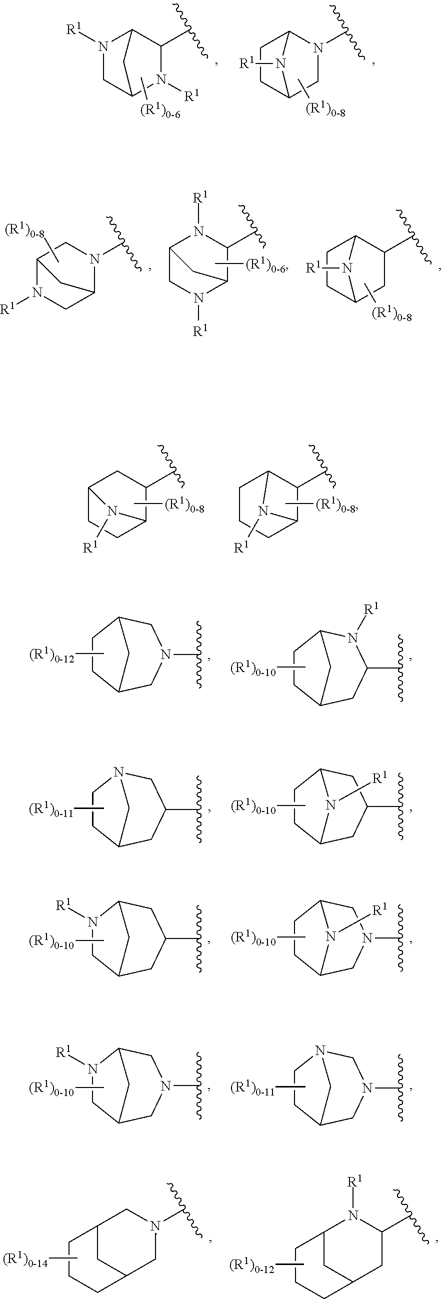

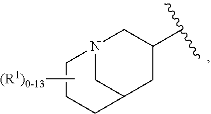

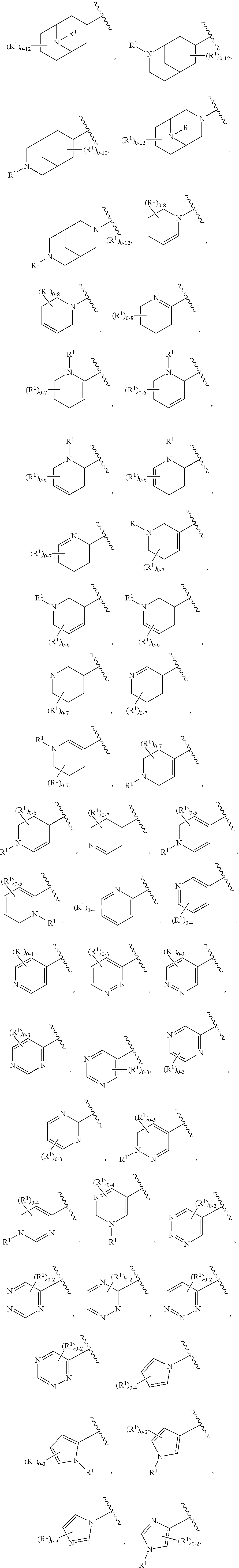

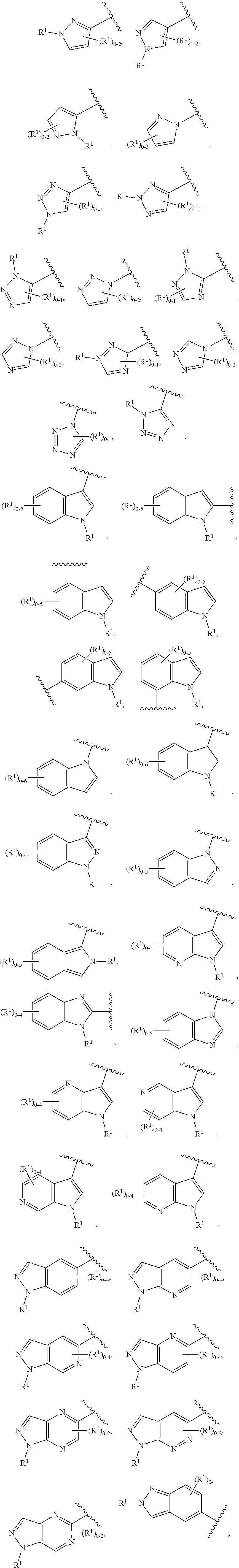

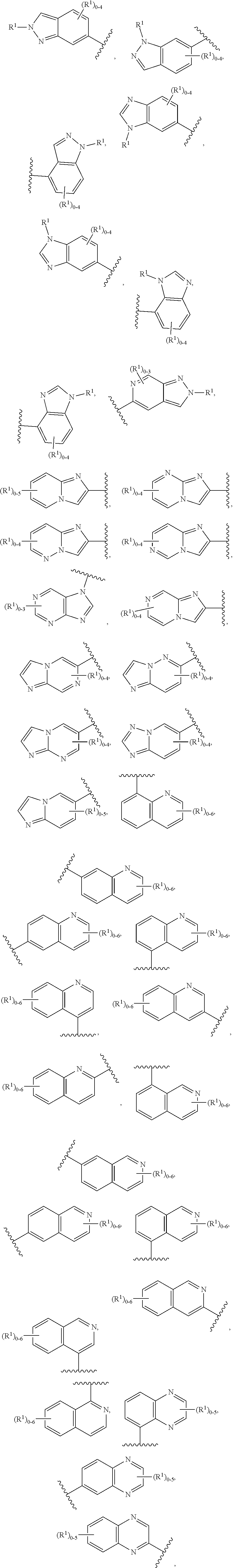

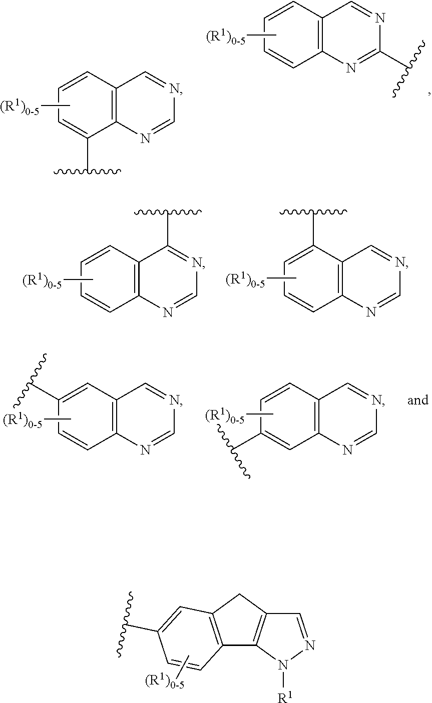

wherein each R¹ is as defined herein. In an embodiment, A is a saturated, partially saturated, or unsaturated (e.g., aromatic) derivative of one of the rings described above. In an embodiment, A is a stereoisomer of one of the rings described above.

In some embodiments, A is selected from:

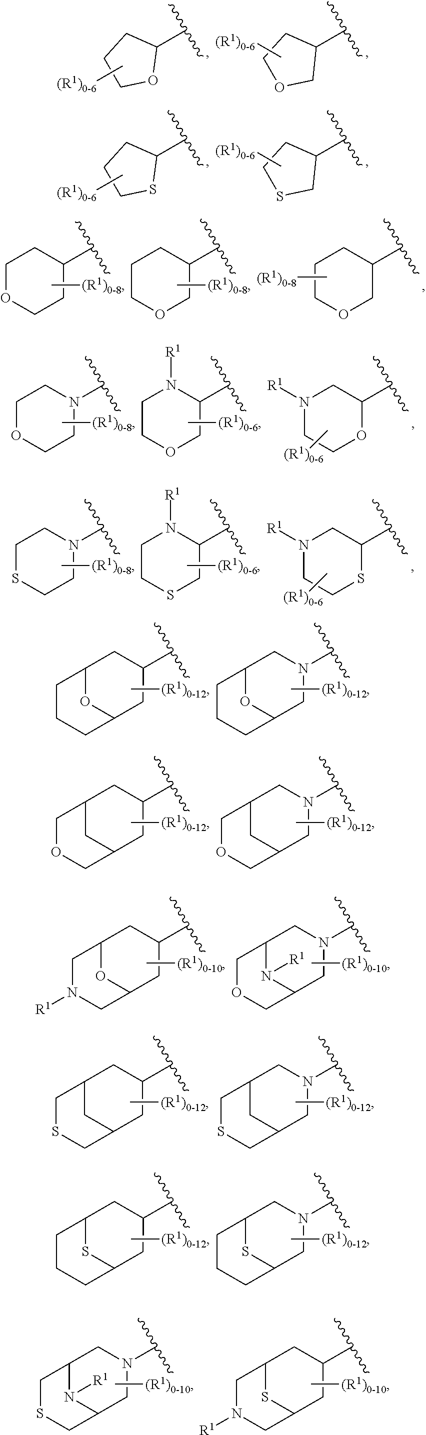

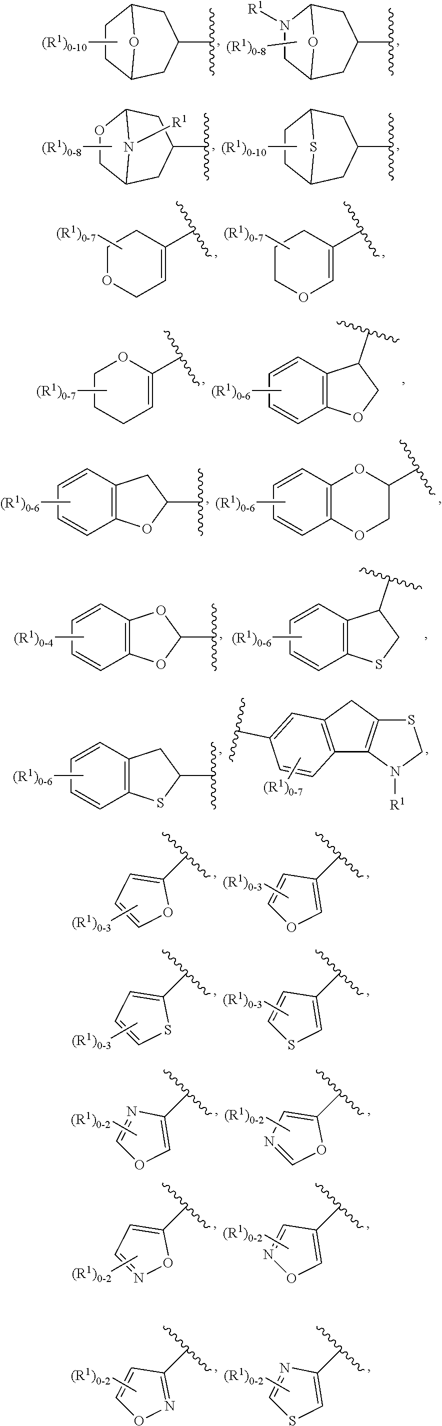

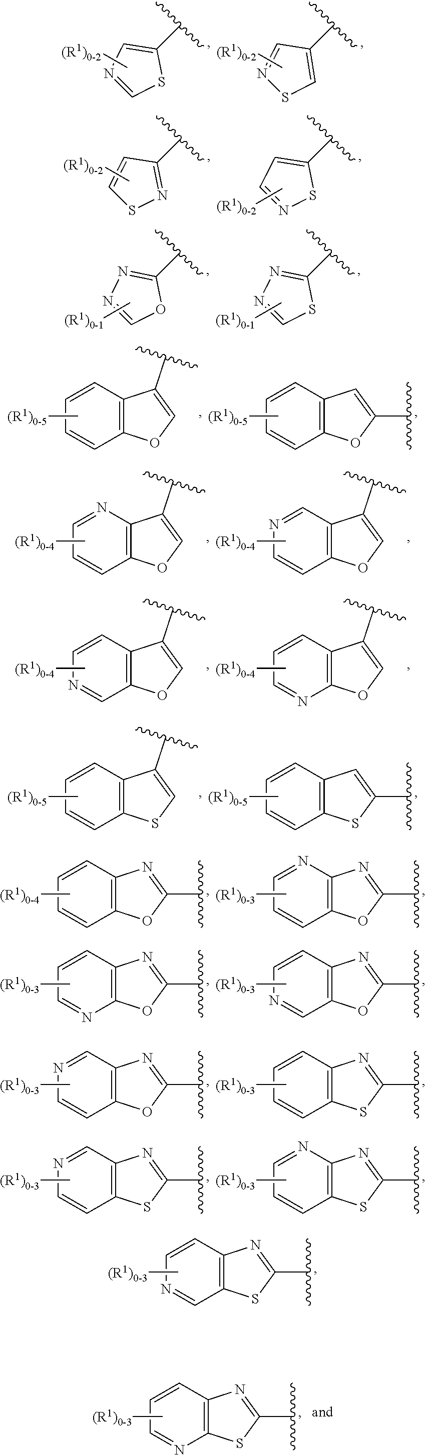

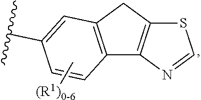

wherein each R¹ is as defined herein. In an embodiment, A is a saturated, partially saturated, or unsaturated (e.g., aromatic) derivative of one of the rings described above. In an embodiment, A is a stereoisomer of one of the rings described above.

In some embodiments, A is selected from

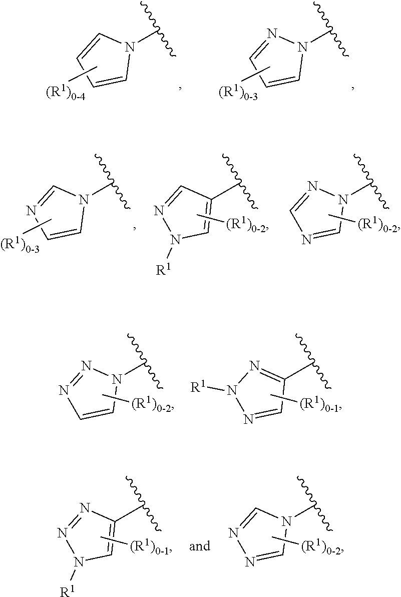

wherein R¹ is as defined herein. In some embodiments, A is selected from

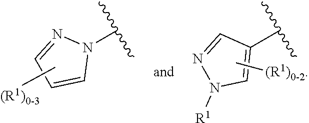

In some embodiments, A is selected from

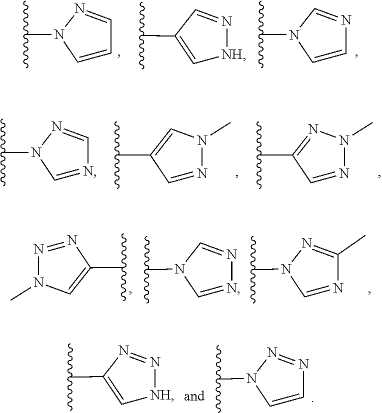

In some embodiments, A is selected from

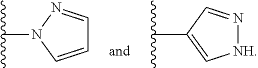 [SMILES: *n1cccn1 ; *c1cn[nH]c1]

In some embodiments A is

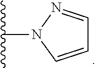 [SMILES: *n1cccn1]

In some embodiments, A is

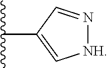 [SMILES: *c1cn[nH]c1]

In some embodiments, A is

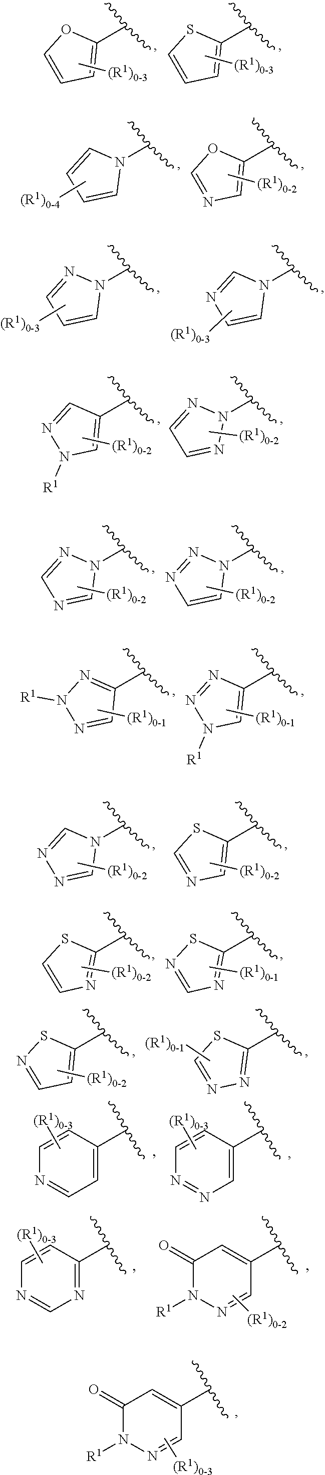

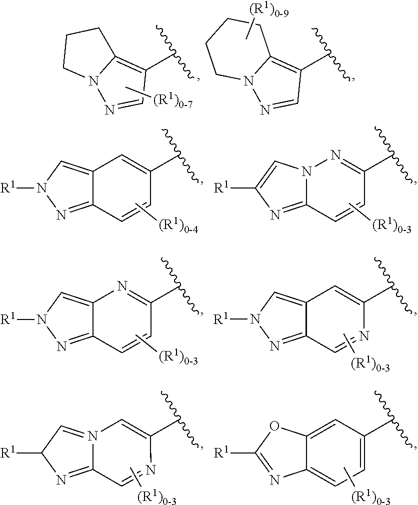

wherein R¹ is as described herein.

In some embodiments, R¹ is hydrogen. In some embodiments, R¹ is C₁-C₆-alkyl. In some embodiments, R¹ is C₂-C₆-alkenyl. In some embodiments, R¹ is C₂-C₆-alkynyl. In some embodiments, R¹ is C₁-C₆-heteroalkyl. In some embodiments, R¹ is C₁-C₆-haloalkyl (e.g., —CF₃). In some embodiments, R¹ is C₁-alkyl (e.g., methyl). In some embodiments, R¹ is unsubstituted C₁-C₆-alkyl, unsubstituted C₂-C₆-alkenyl, unsubstituted C₂-C₆-alkynyl, unsubstituted C₁-C₆-heteroalkyl, or unsubstituted C₁-C₆-haloalkyl. In some embodiments, R¹ is C₁-C₆-alkyl substituted with one or more R⁶. In some embodiments, R¹ is C₂-C₆-alkenyl substituted with one or more R⁶. In some embodiments, R¹ is C₂-C₆-alkynyl substituted with one or more R⁸. In some embodiments, R¹ is C₁-C₆-heteroalkyl substituted with one or more R⁸. In some embodiments, R¹ is C₁-C₆-haloalkyl substituted with one or more R⁸. In some embodiments, R¹ is methyl.

In some embodiments, R¹ is cycloalkyl (e.g., 3-7 membered cycloalkyl). In some embodiments, R¹ is heterocyclyl (e.g., 3-7 membered heterocyclyl). In some embodiments, R¹ is aryl. In some embodiments, R¹ is C₁-C₆ alkylene-aryl (e.g., benzyl). In some embodiments, R¹ is C₁-C₆ alkenylene-aryl. In some embodiments, R¹ is C₁-C₆ alkylene-heteroaryl. In some embodiments, R¹ is heteroaryl. In some embodiments, R¹ is unsubstituted cycloalkyl, unsubstituted heterocyclyl, unsubstituted aryl, unsubstituted C₁-C₆ alkylene-aryl, unsubstituted C₁-C₆ alkenylene-aryl, unsubstituted C₁-C₆ alkylene-heteroaryl, or unsubstituted heteroaryl. In some embodiments, R¹ is cycloalkyl substituted with one or more R⁸. In some embodiments, R¹ is heterocyclyl substituted with one or more R⁸. In some embodiments, R¹ is aryl substituted with one or more R⁸. In some embodiments, R¹ is C₁-C₆ alkylene-aryl substituted with one or more R⁸. In some embodiments, R¹ is C₁-C₆ alkenylene-aryl substituted with one or more R⁸. In some embodiments, R¹ is C₁-C₆ alkylene-heteroaryl substituted with one or more R⁸. In some embodiments, R¹ is heteroaryl substituted with one or more R⁸.

In some embodiments, R¹ is —OR^{A}. In some embodiments, R¹ is —NR^{B}R^{C} (e.g., NH₂ or NMe₂). In some embodiments, R¹ is —NR^{B}C(O)R^{D}. In some embodiments, R¹ is-C(O)NR^{B}R^{C}.

In some embodiments, R¹ is —C(O)R^{D}. In some embodiments, R¹ is —C(O)OR^{D}. In some embodiments, R¹ is —SR^{E}. In some embodiments, R¹ is —S(O)ₓR^{D}. In some embodiments, R¹ is halo, e.g., fluoro, chloro, bromo, or iodo. In some embodiments, R¹ is cyano. In some embodiments, R¹ is nitro (—NO₂). In some embodiments, R¹ is oxo.

In some embodiments, two R¹ groups, together with the atoms to which they are attached, form a 3-7-membered cycloalkyl. In some embodiments, two R¹ groups, together with the atoms to which they are attached, form a 3-7-membered heterocyclyl. In some embodiments, two R¹ groups, together with the atoms to which they are attached, form a 5- or 6-membered aryl. In some embodiments, two R¹ groups, together with the atoms to which they are attached, form a 5- or 6-membered heteroaryl. The cycloalkyl, heterocyclyl, aryl, or heteroaryl may be substituted with one or more R⁸.

In some embodiments, R² is hydrogen. In some embodiments, R² is C₁-C₆ alkyl. In some embodiments, R² is C₂-C₆-alkenyl. In some embodiments, R² is C₂-C₆-alkynyl. In some embodiments, R² is C₁-alkyl (e.g., methyl). In some embodiments, R² is methyl. In some embodiments, R² is —OR^{A}. In some embodiments, R² is halo (e.g., fluoro, chloro, bromo, or iodo). In some embodiments, R² is fluoro. In some embodiments, R² is cyano.

In some embodiments, each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, heteroalkyl, haloalkyl, cycloalkyl, heterocyclyl, aryl, and heteroaryl is optionally substituted with one or more R⁹. In some embodiments, one of R^{4a} and R^{4b} is independently hydrogen and the other of R^{4a} and R^{4b} is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, heteroalkyl, haloalkyl, cycloalkyl, heterocyclyl, aryl, and heteroaryl is optionally substituted with one or more R⁹. In some embodiments, one of R^{4a} and R^{4b} is independently hydrogen and the other of R^{4a} and R^{4b} is independently C₁-C₆-alkyl, C₁-C₆-haloalkyl, or cycloalkyl, wherein each alkyl, heteroalkyl, haloalkyl, cycloalkyl, heterocyclyl, aryl, and heteroaryl is optionally substituted with one or more R⁹. In some embodiments, one of R^{4a} and R^{4b} is independently hydrogen and the other of R^{4a} and R^{4b} is independently C₁-C₆-alkyl (e.g., t-butyl). In some embodiments, one of R^{4a} and R^{4b} is independently hydrogen and the other of R^{4a} and R^{4b} is independently cycloalkyl (e.g., cyclopropyl or cyclobutyl) optionally substituted with one or more R⁹. In some embodiments, one of R^{4a} and R^{4b} is independently hydrogen and the other of R^{4a} and R^{4b} is independently C₁-C₆-haloalkyl. In some embodiments, R^{4a} is hydrogen and R^{4b} is t-butyl, cyclopropyl, CH₂-cyclopropyl, cyclobutyl, 1-methylcylcopropyl, 1-trifluoromethylcyclopropyl, 1-fluoromethylcyclopropyl, and 1-difluoromethylcyclopropyl. In some embodiments,

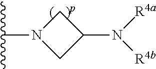

is selected from

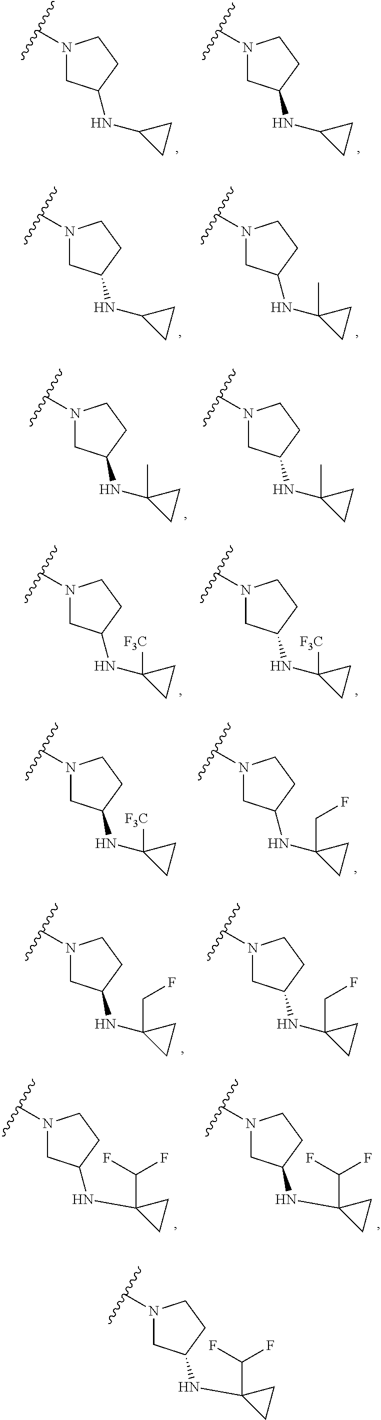

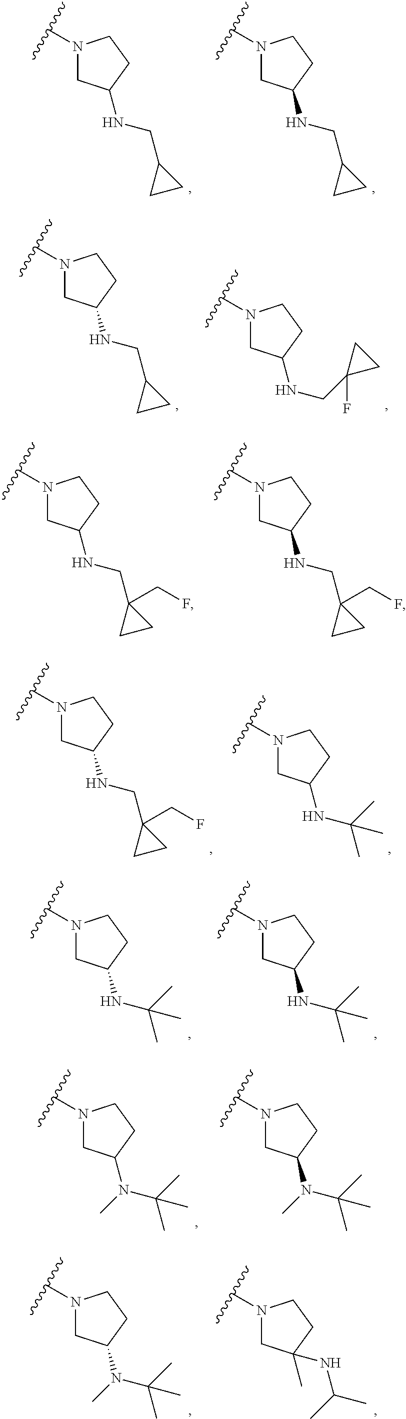

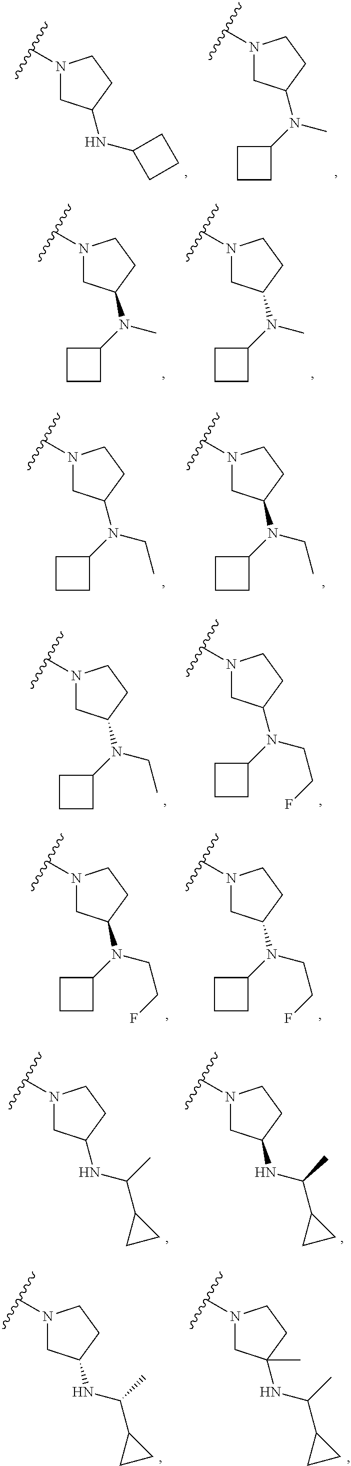

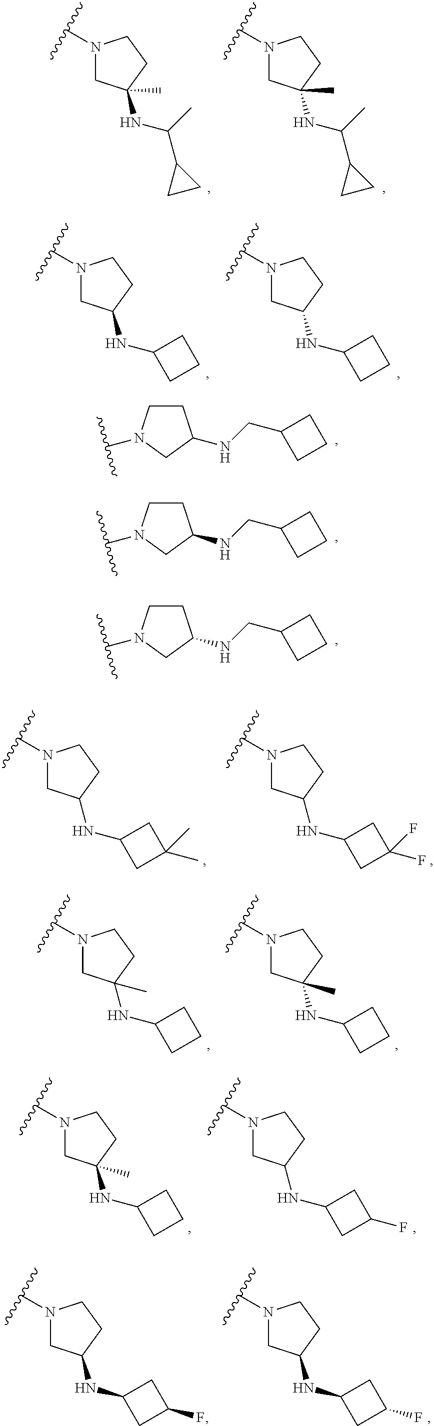

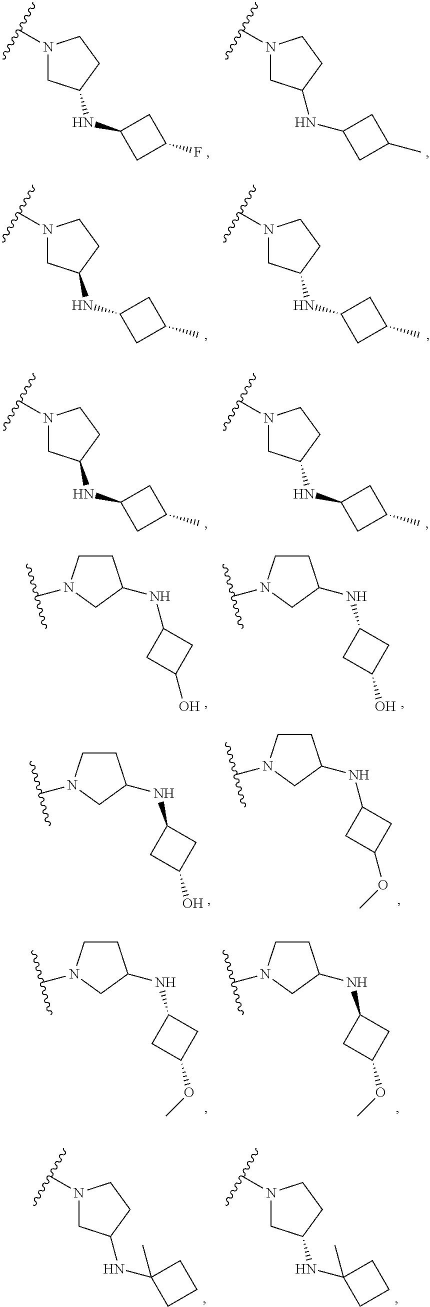

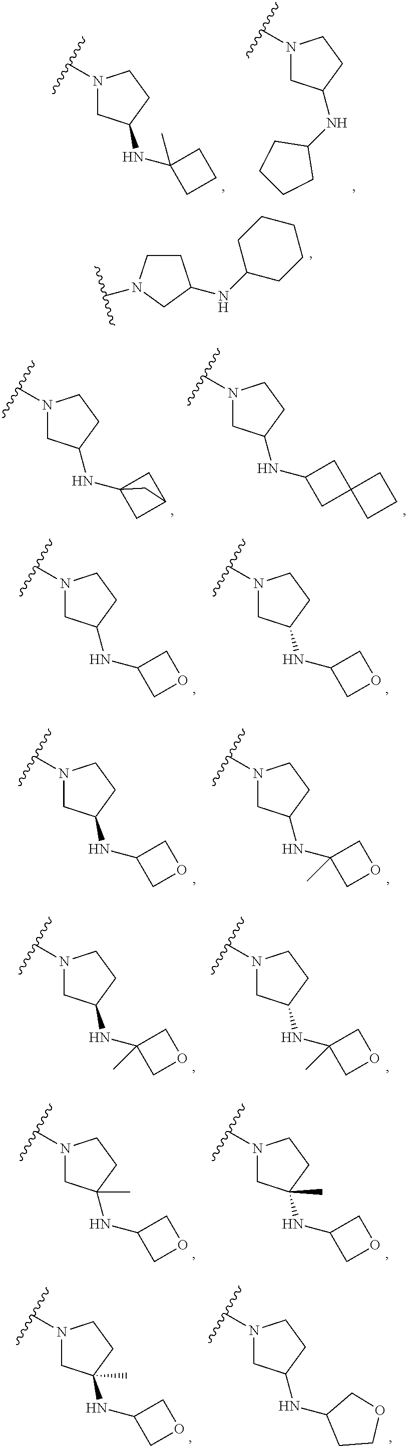

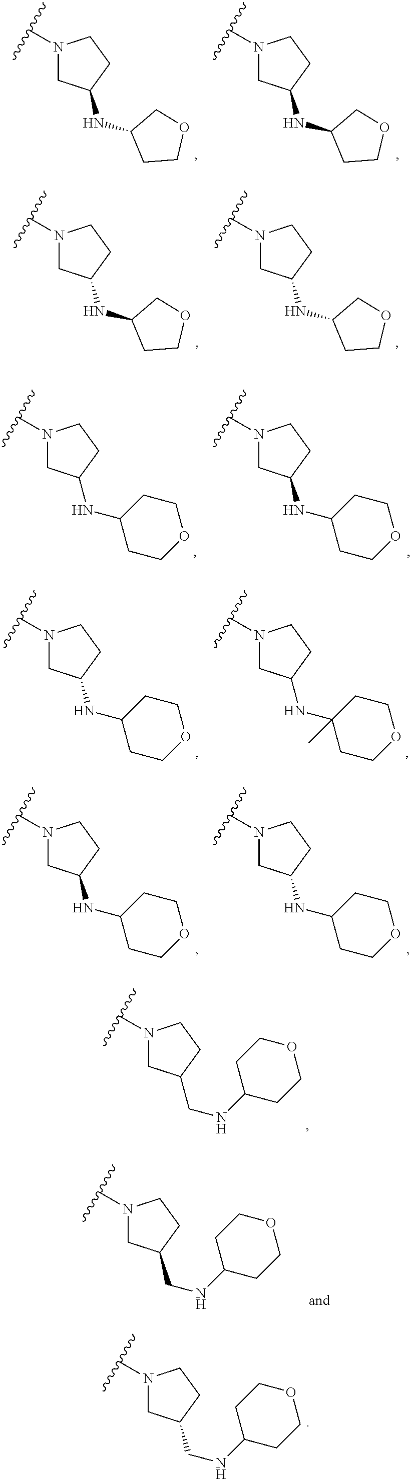

In some embodiments,

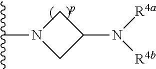

is selected from

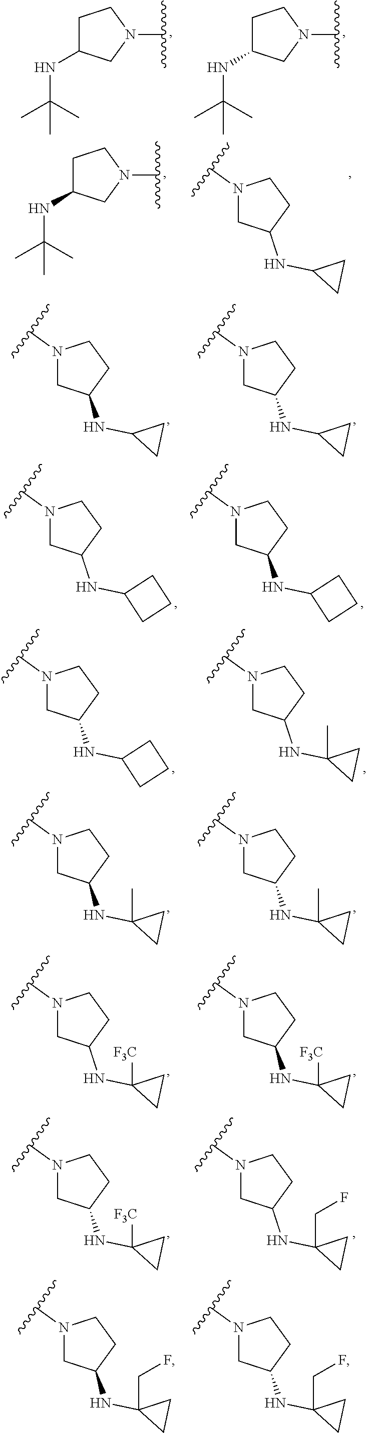

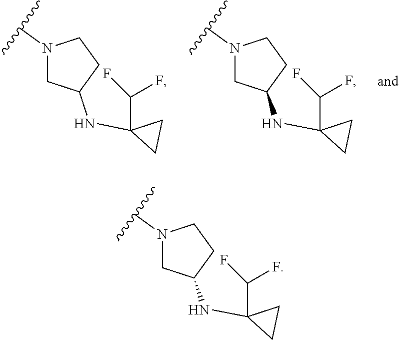 [SMILES: *N1CCC(NC2(C(F)F)CC2)C1 ; *N1CC[C@@H](NC2(C(F)F)CC2)C1 ; *N1CC[C@H](NC2(C(F)F)CC2)C1]

In some embodiments,

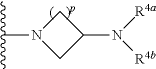

is selected from

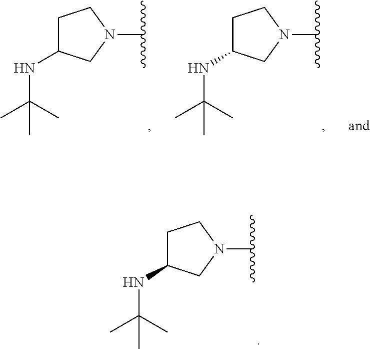 [SMILES: *N1CCC(NC(C)(C)C)C1 ; *N1CC[C@@H](NC(C)(C)C)C1 ; *N1CC[C@H](NC(C)(C)C)C1]

In some embodiments, R⁵ is hydrogen. In some embodiments, R⁵ is C₁-C₆-alkyl.

In some embodiments, R⁶ is hydrogen. In some embodiments, R⁶ is C₁-C₆-alkyl.

In some embodiments, R⁷ is C₂-C₆-alkynyl substituted with one or more R⁷. In some embodiments, one of R⁵ and R⁶ is independently C₁-C₆-heteroalkyl substituted with one or more R⁷. In some embodiments, one of R⁵ and R⁶ is independently C₁-C₆-haloalkyl substituted with one or more R⁷. In some embodiments, one of R⁵ and R⁶ is independently halo, e.g., fluoro, chloro, bromo, or iodo. In some embodiments, one of R⁵ and R⁶ is independently fluoro. In some embodiments, one of R⁵ and R⁶ is independently cyano. In some embodiments, one of R⁵ and R⁶ is independently oxo. In some embodiments, R⁷ is NR^{B}C(O)R^{D}. In some embodiments, one of R⁵ and R⁶ is independently —C(O)NR^{B}R^{C}. In some embodiments, one of R⁵ and R⁶ is independently —C(O)R^{D}.

In some embodiments, R⁸ is C₁-C₆-alkyl. In some embodiments, R⁸ is cycloalkyl. In some embodiments, R⁸ is halo (e.g., fluoro, chloro, bromo, or iodo).

In some embodiments, R^{A}is hydrogen. In some embodiments, R^{A} is C₁-C₆ alkyl (e.g., methyl). In some embodiments, R^{A} is C₁-C₆ haloalkyl. In some embodiments, R^{A} is aryl. In some embodiments, R^{A} is heteroaryl. In some embodiments, R^{A} is C₁-C₆ alkylene-aryl (e.g., benzyl). In some embodiments, R^{A} is C₁-C₆ alkylene-heteroaryl. In some embodiments, R^{A} is C(O)R^{D}. In some embodiments, R^{A} is —S(O)ₓR^{D}.

In some embodiments, R^{B}, R^{C}, or both are each independently hydrogen, C₁-C₆-alkyl, C₁-C₆-heteroalkyl, cycloalkyl, heterocyclyl, or —OR^{A}. In some embodiments, each of R^{B} and R^{C} is independently hydrogen. In some embodiments, each of R^{B} and R^{C} is independently C₁-C₆ alkyl. In some embodiments, one of R^{B} and R^{C} is hydrogen, and the other of R^{B} and R^{C} is C₁-C₆ alkyl. In some embodiments, R^{B} and R^{C} together with the atom to which they are attached form a 3-7-membered heterocyclyl ring optionally substituted with one or more of R⁸ (e.g., 1, 2, or 3 R⁸).

In some embodiments, R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl (e.g., benzyl), or C₁-C₆ alkylene-heteroaryl. In some embodiments, R^{D} is C₁-C₆ alkyl. In some embodiments, R^{D} is hydrogen. In some embodiments, R^{D} is heterocyclyl. In some embodiments, R^{D} is aryl. In some embodiments, R^{D} is heteroaryl. In some embodiments, R^{D} is C₁-C₆ alkylene-aryl (e.g., benzyl). In some embodiments, R^{D} is C₁-C₆ alkylene-heteroaryl.

In some embodiments, p is 1. In some embodiments, p is 2. In some embodiments, p is 3. In some embodiments, x is an integer between 0 and 2 (e.g., 0, 1, or 2). In some embodiments, x is 0. In some embodiments, x is 1. In some embodiments, x is 2.

In some embodiments, the compound of Formula (I) is a compound of Formula (I-a):

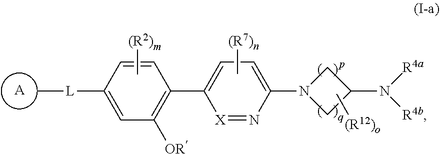

or a pharmaceutically acceptable salt, solvate, hydrate, tautomer, or stereoisomer thereof, wherein A is heteroaryl optionally substituted with one or more R¹; L is absent, —O—, —C(O)—, —N(R³)—; X is C(R⁵) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² and R⁷ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D} NR^{B}C(O)R^{D}, or —C(O)NR^{B}R^{C}; each R³ is independently hydrogen, C₁-C₆-alkyl, C₁-C₆-haloalkyl, or cycloalkyl; each of R^{4a} and R^{4b} is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R⁹; R⁵ is hydrogen, C₁-C₆-alkyl, C₁-C₆-haloalkyl, or C(O)R^{D}; R⁶ is hydrogen or C₁-C₆-alkyl; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R⁹ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}; each R^{A} is independently hydrogen, C₁-C₆ alkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, 2, 3, or 4; n is 0, 1, or 2; p and q are each independently 1, 2, 3, or 4; o is 0, 1, 2, 3, 4, 5, 6, 7, 8, 9, 10, 11, 12, or 13; and x is 0, 1, or 2.

In some embodiments, the compound of Formula (I) is a compound of Formula (I-b):

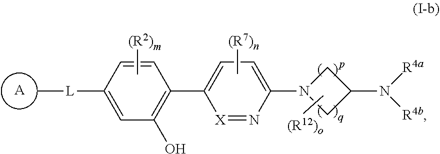

or a pharmaceutically acceptable salt, solvate, hydrate, tautomer, or stereoisomer thereof, wherein A is heteroaryl optionally substituted with one or more R¹; L is absent, —O—, —C(O)—, —N(R³)—; X is C(R⁵) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² and R⁷ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D} NR^{B}C(O)R^{D}, or —C(O)NR^{B}R^{C}; each R³ is independently hydrogen, C₁-C₆-alkyl, C₁-C₆-haloalkyl, or cycloalkyl; each of R^{4a} and R^{4b} is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R⁹; R⁵ is hydrogen, C₁-C₆-alkyl, C₁-C₆-haloalkyl, or C(O)R^{D}; R′ is hydrogen, C₁-C₆-alkyl or cycloalkyl; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R⁹ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}; each R^{A}is independently hydrogen, C₁-C₆ alkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, 2, 3, or 4; n is 0, 1, or 2; p and q are each independently 1, 2, 3, or 4; o is 0, 1, 2, 3, 4, 5, 6, 7, 8, 9, 10, 11, 12, or 13; and x is 0, 1, or 2.

In some embodiments, the compound of Formula (I) is a compound of Formula (I-c):

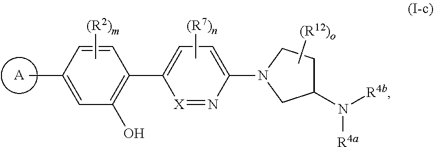

or a pharmaceutically acceptable salt, solvate, hydrate, tautomer, or stereoisomer thereof, wherein A is heteroaryl optionally substituted with one or more R¹; X is C(R⁵) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² and R⁷ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D}, NR^{B}C(O)R^{D}, or —C(O)NR^{B}R^{C}; each of R^{4a} and R^{4b} is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R⁹; R⁵ is hydrogen, C₁-C₆-alkyl, C₁-C₆-haloalkyl, or C(O)R^{D}; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R⁹ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}; each R^{A} is independently hydrogen, C₁-C₆ alkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, 2, 3, or 4; o is 0, 1, 2, 3, 4, 5, 6, or 7; n is 0, 1, or 2; and x is 0, 1, or 2.

In some embodiments, the compound of Formula (I) is a compound of Formula (I-d):

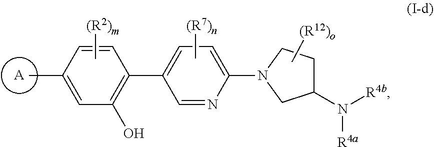

or a pharmaceutically acceptable salt, solvate, hydrate, tautomer, or stereoisomer thereof, wherein A is heteroaryl optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² and R⁷ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D}NR^{B}C(O)R^{D}, or —C(O)NR^{B}R^{C}; each of R^{4a} and R^{4b} is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R⁹; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R⁹ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}; each R^{A} is independently hydrogen, C₁-C₆ alkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, 2, 3, or 4; n is 0, 1, or 2; o is 0, 1, 2, 3, 4, 5, 6, or 7; and x is 0, 1, or 2.

In some embodiments, R^{4a} is hydrogen and R^{4b} is t-butyl, cyclopropyl, CH₂-cyclopropyl, cyclobutyl, 1-methylcylcopropyl, 1-trifluoromethylcyclopropyl, 1-fluoromethylcyclopropyl, and 1-difluoromethylcyclopropyl.

In some embodiments, the compound of Formula (I) is a compound of Formula (I-e):

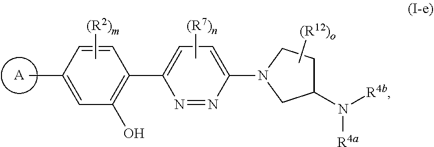

or a pharmaceutically acceptable salt, solvate, hydrate, tautomer, or stereoisomer thereof, wherein A is heteroaryl optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² and R⁷ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D}NR^{B}C(O)R^{D}, or —C(O)NR^{B}R^{C}; each of R^{4a} and R^{4b} is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R⁹; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R⁹ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}; each R^{A} is independently hydrogen, C₁-C₆ alkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, 2, 3, or 4; n is 0, 1, or 2; o is 0, 1, 2, 3, 4, 5, 6, or 7; and x is 0, 1, or 2.

In some embodiments, R^{4a} is hydrogen and R^{4b} is t-butyl, cyclopropyl, CH₂-cyclopropyl, cyclobutyl, 1-methylcylcopropyl, 1-trifluoromethylcyclopropyl, 1-fluoromethylcyclopropyl, and 1-difluoromethylcyclopropyl.

In some embodiments, the compound of Formula (I) is a compound of Formula (I-f):

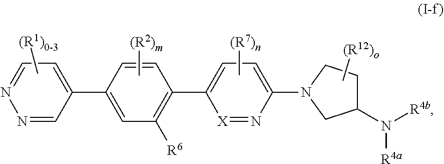

or a pharmaceutically acceptable salt, solvate, hydrate, tautomer, or stereoisomer thereof, wherein X is C(R⁵) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² and R⁷ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D} NR^{B}C(O)R^{D}, or —C(O)NR^{B}R^{C}; each of R^{4a} and R^{4b} is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R⁹; R⁵ is hydrogen, C₁-C₆-alkyl, C₁-C₆-haloalkyl, or C(O)R^{D}; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R⁹ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}—C(O)NR^{B}R^{C}, —C(O)R^{D}, or —S(O)ₓR^{D}; each R^{A}is independently hydrogen, C₁-C₆ alkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, 2, 3, or 4; n is 0, 1, or 2; o is 0, 1, 2, 3, 4, 5, 6, or 7; and x is 0, 1, or 2.

In some embodiments, R^{4a} is hydrogen and R^{4b} is t-butyl, cyclopropyl, CH₂-cyclopropyl, cyclobutyl, 1-methylcylcopropyl, 1-trifluoromethylcyclopropyl, 1-fluoromethylcyclopropyl, and 1-difluoromethylcyclopropyl.

In some embodiments, the compound of Formula (I) is a compound of Formula (I-g):

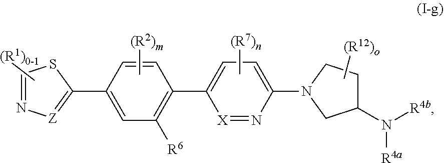

or a pharmaceutically acceptable salt, solvate, hydrate, tautomer, or stereoisomer thereof, wherein X is C(R⁵) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² and R⁷ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D} NR^{B}C(O)R^{D}, or —C(O)NR^{B}R^{C}; each of R^{4a} and R^{4b} is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R⁹; R⁵ is hydrogen, C₁-C₆-alkyl, C₁-C₆-haloalkyl, or C(O)R^{D}; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R⁹ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}—C(O)NR^{B}R^{C}, —C(O)R^{D}, or —S(O)ₓR^{D}; each R^{A}is independently hydrogen, C₁-C₆ alkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, 2, 3, or 4; o is 0, 1, 2, 3, 4, 5, 6, or 7; n is 0, 1, or 2; and x is 0, 1, or 2.

In some embodiments, R^{4a} is hydrogen and R^{4b} is t-butyl, cyclopropyl, CH₂-cyclopropyl, cyclobutyl, 1-methylcylcopropyl, 1-trifluoromethylcyclopropyl, 1-fluoromethylcyclopropyl, and 1-difluoromethylcyclopropyl.

In some embodiments, the compound of Formula (I) is a compound of Formula (I-h):

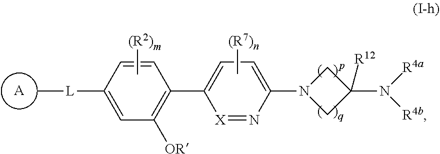

or a pharmaceutically acceptable salt, solvate, hydrate, tautomer, or stereoisomer thereof, wherein A is heteroaryl optionally substituted with one or more R¹; L is absent, —O—, —C(O)—, —N(R³)—; X is C(R⁵) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² and R⁷ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D} NR^{B}C(O)R^{D}, or —C(O)NR^{B}R^{C}; each R³ is independently hydrogen, C₁-C₆-alkyl, C₁-C₆-haloalkyl, or cycloalkyl; each of R^{4a} and R^{4b} is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R⁹; R⁵ is hydrogen, C₁-C₆-alkyl, C₁-C₆-haloalkyl, or C(O)R^{D}; R⁶ is hydrogen or C₁-C₆-alkyl; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R⁹ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}; each R^{A} is independently hydrogen, C₁-C₆ alkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, 2, 3, or 4; n is 0, 1, or 2; p and q are each independently 1, 2, 3, or 4; and x is 0, 1, or 2.

In some embodiments, the compound of Formula (I) is selected from a compound in Table 1, or a pharmaceutically acceptable salt thereof.

TABLE 1
Exemplary compounds of Formula (I)

| Compound No. | Structure |
|---|---|
| 100 | 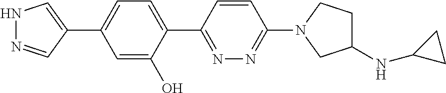 [SMILES: Oc1cc(-c2cn[nH]c2)ccc1-c1ccc(N2CCC(NC3CC3)C2)nn1] |
| 101 | 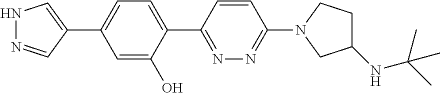 [SMILES: CC(C)(C)NC1CCN(c2ccc(-c3ccc(-c4cn[nH]c4)cc3O)nn2)C1] |
| 102 | 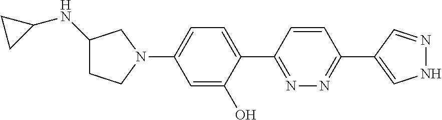 [SMILES: Oc1cc(N2CCC(NC3CC3)C2)ccc1-c1ccc(-c2cn[nH]c2)nn1] |
| 103 | 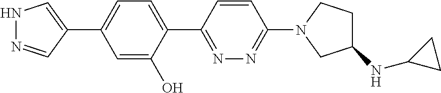 [SMILES: Oc1cc(-c2cn[nH]c2)ccc1-c1ccc(N2CC[C@@H](NC3CC3)C2)nn1] |
| 104 | 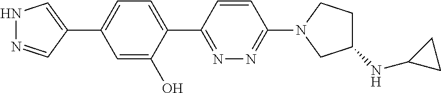 [SMILES: Oc1cc(-c2cn[nH]c2)ccc1-c1ccc(N2CC[C@H](NC3CC3)C2)nn1] |
| 105 | 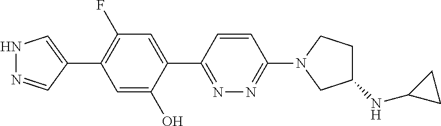 [SMILES: Oc1cc(-c2cn[nH]c2)c(F)cc1-c1ccc(N2CC[C@H](NC3CC3)C2)nn1] |
| 106 | 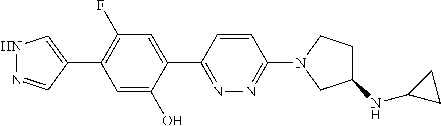 [SMILES: Oc1cc(-c2cn[nH]c2)c(F)cc1-c1ccc(N2CC[C@@H](NC3CC3)C2)nn1] |
| 107 | 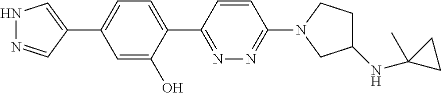 [SMILES: CC1(NC2CCN(c3ccc(-c4ccc(-c5cn[nH]c5)cc4O)nn3)C2)CC1] |
| 108 | 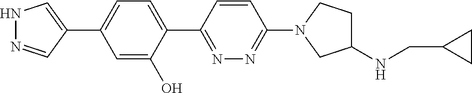 [SMILES: Oc1cc(-c2cn[nH]c2)ccc1-c1ccc(N2CCC(NCC3CC3)C2)nn1] |
| 109 | 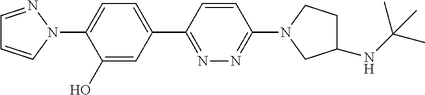 [SMILES: CC(C)(C)NC1CCN(c2ccc(-c3ccc(-n4cccn4)c(O)c3)nn2)C1] |
| 110 | 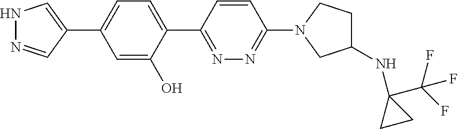 [SMILES: Oc1cc(-c2cn[nH]c2)ccc1-c1ccc(N2CCC(NC3(C(F)(F)F)CC3)C2)nn1] |
| 111 | 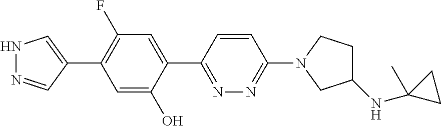 [SMILES: CC1(NC2CCN(c3ccc(-c4cc(F)c(-c5cn[nH]c5)cc4O)nn3)C2)CC1] |
| 112 | 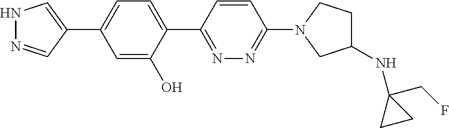 [SMILES: Oc1cc(-c2cn[nH]c2)ccc1-c1ccc(N2CCC(NC3(CF)CC3)C2)nn1] |
| 113 | 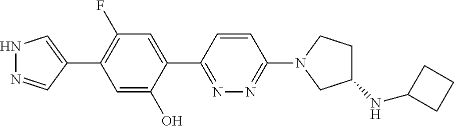 [SMILES: Oc1cc(-c2cn[nH]c2)c(F)cc1-c1ccc(N2CC[C@H](NC3CCC3)C2)nn1] |
| 114 | 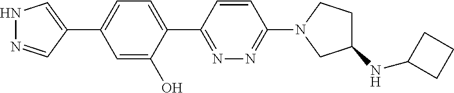 [SMILES: Oc1cc(-c2cn[nH]c2)ccc1-c1ccc(N2CC[C@@H](NC3CCC3)C2)nn1] |
| 115 | 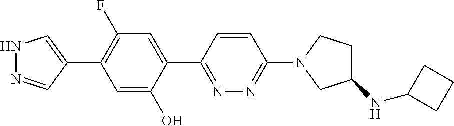 [SMILES: Oc1cc(-c2cn[nH]c2)c(F)cc1-c1ccc(N2CC[C@@H](NC3CCC3)C2)nn1] |
| 116 | 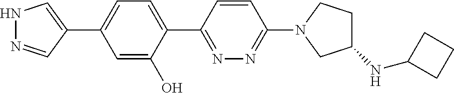 [SMILES: Oc1cc(-c2cn[nH]c2)ccc1-c1ccc(N2CC[C@H](NC3CCC3)C2)nn1] |
| 117 | 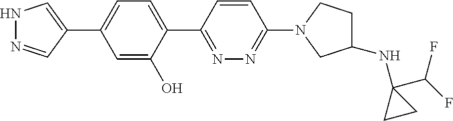 [SMILES: Oc1cc(-c2cn[nH]c2)ccc1-c1ccc(N2CCC(NC3(C(F)F)CC3)C2)nn1] |
| 118 | 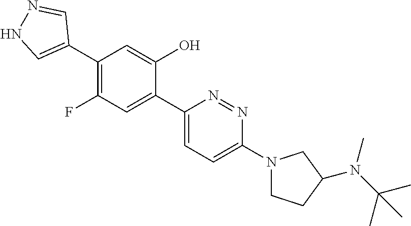 [SMILES: CN(C1CCN(c2ccc(-c3cc(F)c(-c4cn[nH]c4)cc3O)nn2)C1)C(C)(C)C] |
| 119 | 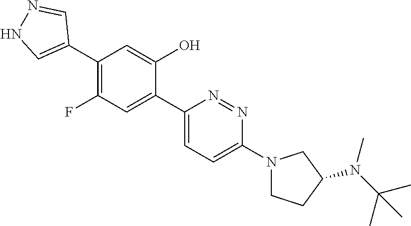 [SMILES: CN([C@@H]1CCN(c2ccc(-c3cc(F)c(-c4cn[nH]c4)cc3O)nn2)C1)C(C)(C)C] |
| 120 | 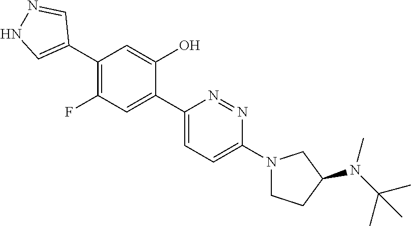 [SMILES: CN([C@H]1CCN(c2ccc(-c3cc(F)c(-c4cn[nH]c4)cc3O)nn2)C1)C(C)(C)C] |
| 121 | 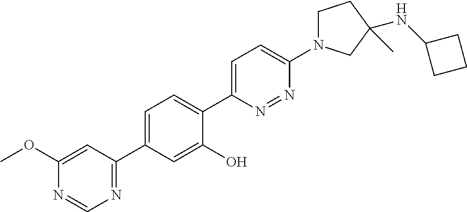 [SMILES: COc1cc(-c2ccc(-c3ccc(N4CCC(C)(NC5CCC5)C4)nn3)c(O)c2)ncn1] |
| 122 | 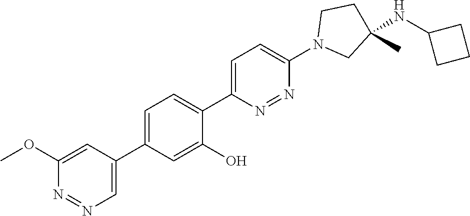 [SMILES: COc1cc(-c2ccc(-c3ccc(N4CC[C@](C)(NC5CCC5)C4)nn3)c(O)c2)cnn1] |
| 123 | 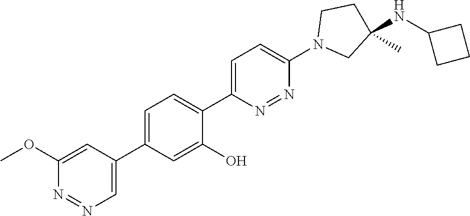 [SMILES: COc1cc(-c2ccc(-c3ccc(N4CC[C@@](C)(NC5CCC5)C4)nn3)c(O)c2)cnn1] |
| 124 | 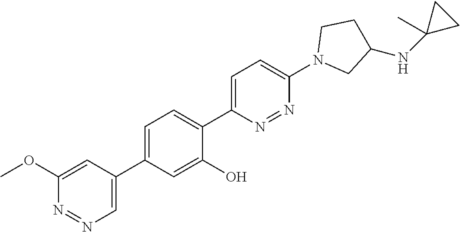 [SMILES: COc1cc(-c2ccc(-c3ccc(N4CCC(NC5(C)CC5)C4)nn3)c(O)c2)cnn1] |
| 125 | 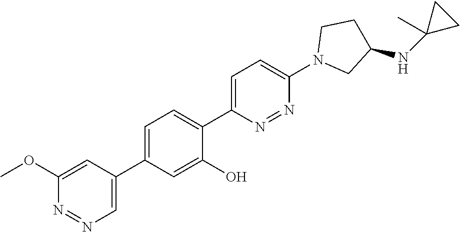 [SMILES: COc1cc(-c2ccc(-c3ccc(N4CC[C@@H](NC5(C)CC5)C4)nn3)c(O)c2)cnn1] |
| 126 | 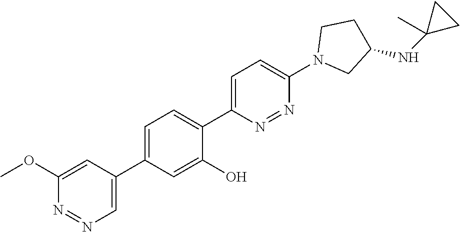 [SMILES: COc1cc(-c2ccc(-c3ccc(N4CC[C@H](NC5(C)CC5)C4)nn3)c(O)c2)cnn1] |
| 127 | 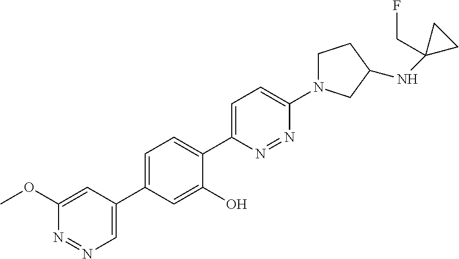 [SMILES: COc1cc(-c2ccc(-c3ccc(N4CCC(NC5(CF)CC5)C4)nn3)c(O)c2)cnn1] |
| 128 | 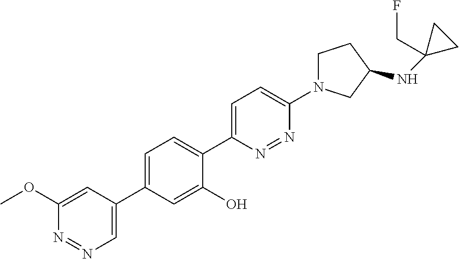 [SMILES: COc1cc(-c2ccc(-c3ccc(N4CC[C@@H](NC5(CF)CC5)C4)nn3)c(O)c2)cnn1] |
| 129 | 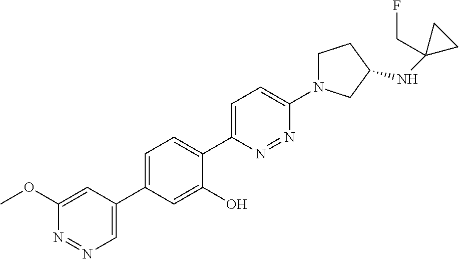 [SMILES: COc1cc(-c2ccc(-c3ccc(N4CC[C@H](NC5(CF)CC5)C4)nn3)c(O)c2)cnn1] |
| 130 | 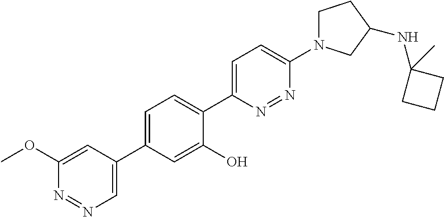 [SMILES: COc1cc(-c2ccc(-c3ccc(N4CCC(NC5(C)CCC5)C4)nn3)c(O)c2)cnn1] |
| 131 | 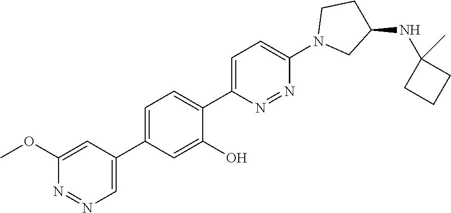 [SMILES: COc1cc(-c2ccc(-c3ccc(N4CC[C@@H](NC5(C)CCC5)C4)nn3)c(O)c2)cnn1] |
| 132 | 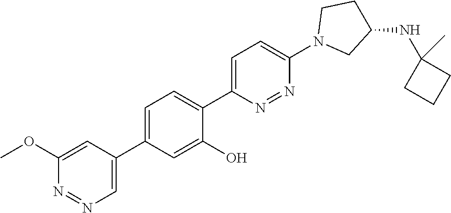 [SMILES: COc1cc(-c2ccc(-c3ccc(N4CC[C@H](NC5(C)CCC5)C4)nn3)c(O)c2)cnn1] |
| 133 | 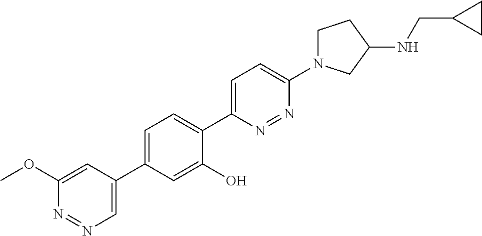 [SMILES: COc1cc(-c2ccc(-c3ccc(N4CCC(NCC5CC5)C4)nn3)c(O)c2)cnn1] |
| 134 | 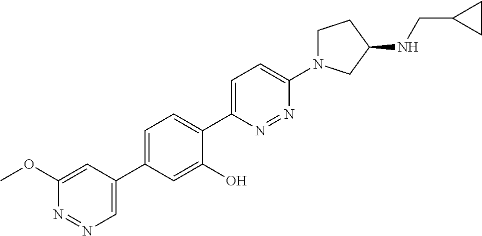 [SMILES: COc1cc(-c2ccc(-c3ccc(N4CC[C@@H](NCC5CC5)C4)nn3)c(O)c2)cnn1] |
| 135 | 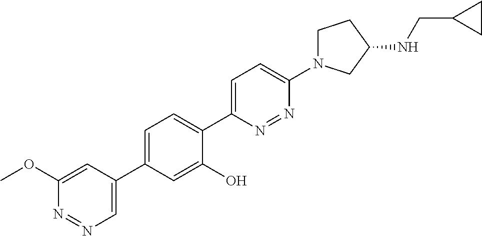 [SMILES: COc1cc(-c2ccc(-c3ccc(N4CC[C@H](NCC5CC5)C4)nn3)c(O)c2)cnn1] |
| 136 | 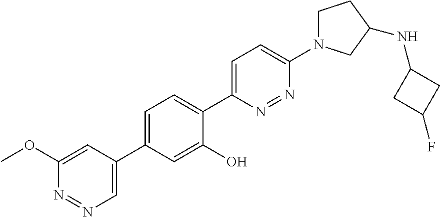 [SMILES: COc1cc(-c2ccc(-c3ccc(N4CCC(NC5CC(F)C5)C4)nn3)c(O)c2)cnn1] |
| 137 | 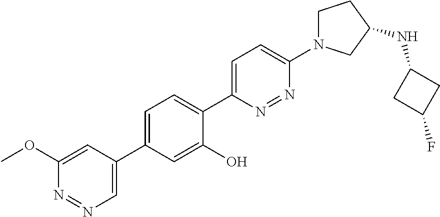 [SMILES: COc1cc(-c2ccc(-c3ccc(N4CC[C@H](N[C@H]5C[C@@H](F)C5)C4)nn3)c(O)c2)cnn1] |
| 138 | 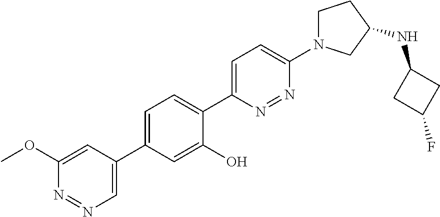 [SMILES: COc1cc(-c2ccc(-c3ccc(N4CC[C@H](N[C@H]5C[C@H](F)C5)C4)nn3)c(O)c2)cnn1] |
| 139 | 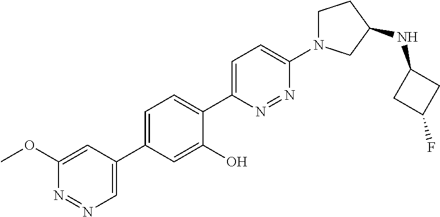 [SMILES: COc1cc(-c2ccc(-c3ccc(N4CC[C@@H](N[C@H]5C[C@H](F)C5)C4)nn3)c(O)c2)cnn1] |
| 140 | 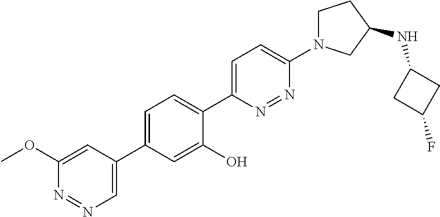 [SMILES: COc1cc(-c2ccc(-c3ccc(N4CC[C@@H](N[C@H]5C[C@@H](F)C5)C4)nn3)c(O)c2)cnn1] |
| 141 | 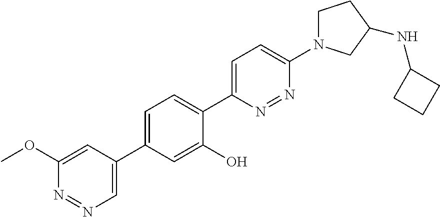 [SMILES: COc1cc(-c2ccc(-c3ccc(N4CCC(NC5CCC5)C4)nn3)c(O)c2)cnn1] |
| 142 | 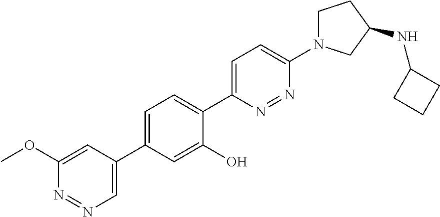 [SMILES: COc1cc(-c2ccc(-c3ccc(N4CC[C@@H](NC5CCC5)C4)nn3)c(O)c2)cnn1] |
| 143 | 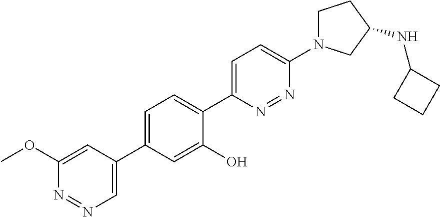 [SMILES: COc1cc(-c2ccc(-c3ccc(N4CC[C@H](NC5CCC5)C4)nn3)c(O)c2)cnn1] |
| 144 | 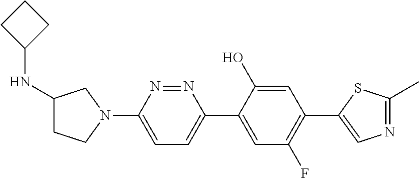 [SMILES: Cc1ncc(-c2cc(O)c(-c3ccc(N4CCC(NC5CCC5)C4)nn3)cc2F)s1] |
| 145 | 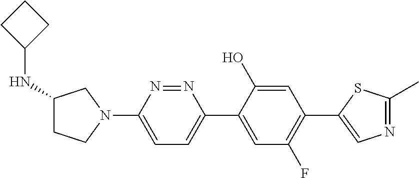 [SMILES: Cc1ncc(-c2cc(O)c(-c3ccc(N4CC[C@H](NC5CCC5)C4)nn3)cc2F)s1] |
| 146 | 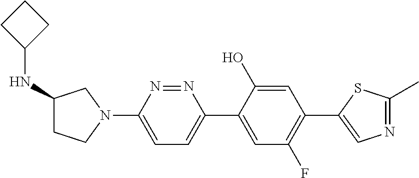 [SMILES: Cc1ncc(-c2cc(O)c(-c3ccc(N4CC[C@@H](NC5CCC5)C4)nn3)cc2F)s1] |
| 147 | 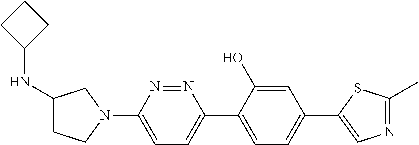 [SMILES: Cc1ncc(-c2ccc(-c3ccc(N4CCC(NC5CCC5)C4)nn3)c(O)c2)s1] |
| 148 | 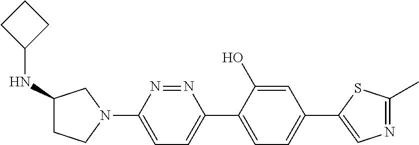 [SMILES: Cc1ncc(-c2ccc(-c3ccc(N4CC[C@@H](NC5CCC5)C4)nn3)c(O)c2)s1] |
| 149 | 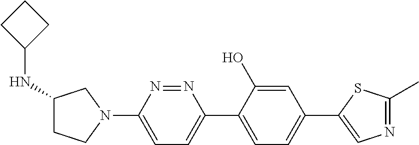 [SMILES: Cc1ncc(-c2ccc(-c3ccc(N4CC[C@H](NC5CCC5)C4)nn3)c(O)c2)s1] |
| 151 | 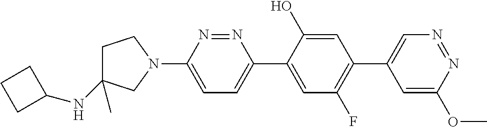 [SMILES: COc1cc(-c2cc(O)c(-c3ccc(N4CCC(C)(NC5CCC5)C4)nn3)cc2F)cnn1] |
| 152 | 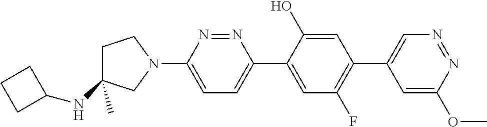 [SMILES: COc1cc(-c2cc(O)c(-c3ccc(N4CC[C@](C)(NC5CCC5)C4)nn3)cc2F)cnn1] |
| 153 | 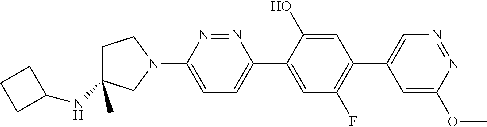 [SMILES: COc1cc(-c2cc(O)c(-c3ccc(N4CC[C@@](C)(NC5CCC5)C4)nn3)cc2F)cnn1] |
| 154 | 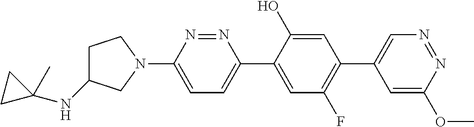 [SMILES: COc1cc(-c2cc(O)c(-c3ccc(N4CCC(NC5(C)CC5)C4)nn3)cc2F)cnn1] |
| 155 | 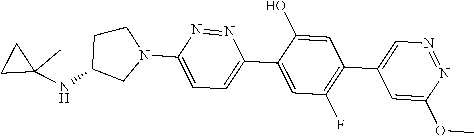 |
| 156 | 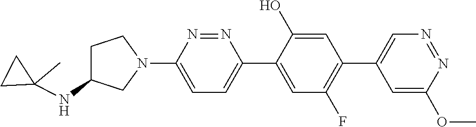 |
| 157 | 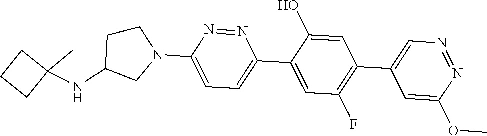 |
| 158 | 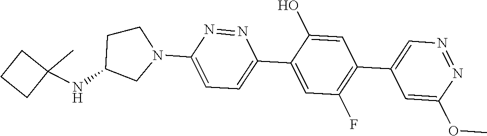 |
| 159 | 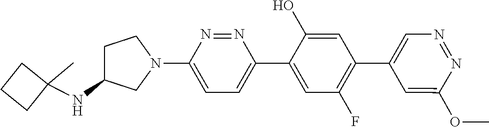 |
| 160 | 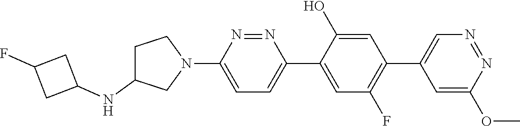 [SMILES: COc1cc(-c2cc(O)c(-c3ccc(N4CCC(NC5CC(F)C5)C4)nn3)cc2F)cnn1] |
| 161 | 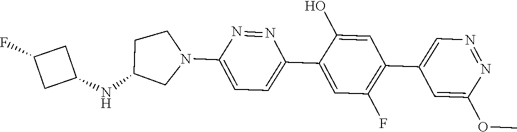 [SMILES: COc1cc(-c2cc(O)c(-c3ccc(N4CC[C@@H](N[C@H]5C[C@@H](F)C5)C4)nn3)cc2F)cnn1] |
| 162 | 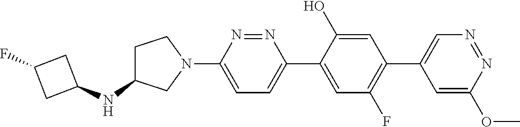 [SMILES: COc1cc(-c2cc(O)c(-c3ccc(N4CC[C@H](N[C@H]5C[C@H](F)C5)C4)nn3)cc2F)cnn1] |
| 163 | 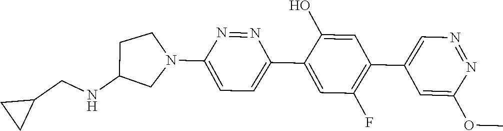 |
| 164 | 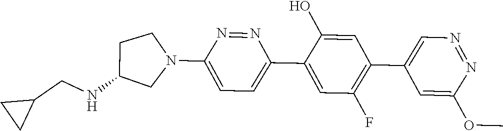 |
| 165 | 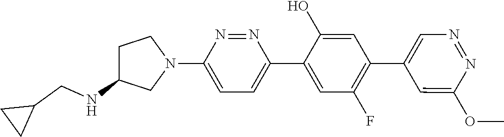 |
| 166 | 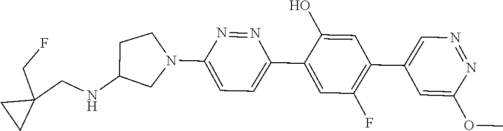 |
| 167 | 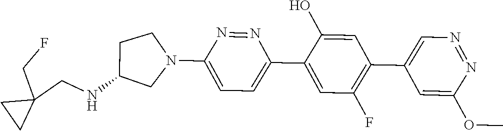 |
| 168 | 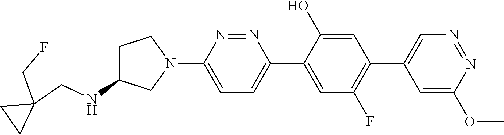 |
| 170 | 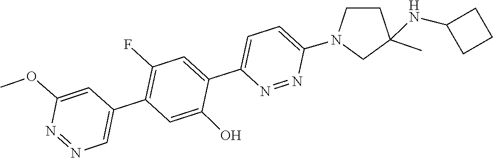 [SMILES: COc1cc(-c2cc(O)c(-c3ccc(N4CCC(C)(NC5CCC5)C4)nn3)cc2F)cnn1] |
| 171 | 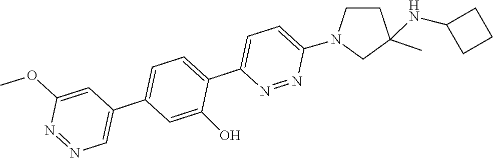 [SMILES: COc1cc(-c2ccc(-c3ccc(N4CCC(C)(NC5CCC5)C4)nn3)c(O)c2)cnn1] |
| 172 | 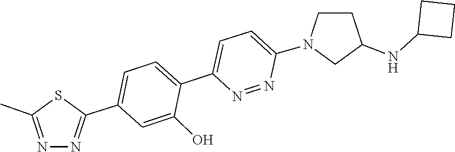 [SMILES: Cc1nnc(-c2ccc(-c3ccc(N4CCC(NC5CCC5)C4)nn3)c(O)c2)s1] |
| 173 | 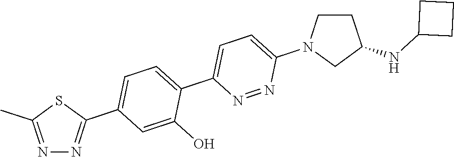 [SMILES: Cc1nnc(-c2ccc(-c3ccc(N4CC[C@H](NC5CCC5)C4)nn3)c(O)c2)s1] |
| 174 | 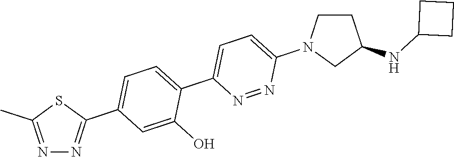 [SMILES: Cc1nnc(-c2ccc(-c3ccc(N4CC[C@@H](NC5CCC5)C4)nn3)c(O)c2)s1] |
| 175 | 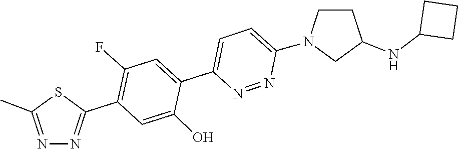 [SMILES: Cc1nnc(-c2cc(O)c(-c3ccc(N4CCC(NC5CCC5)C4)nn3)cc2F)s1] |
| 176 | 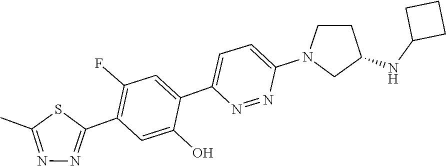 [SMILES: Cc1nnc(-c2cc(O)c(-c3ccc(N4CC[C@H](NC5CCC5)C4)nn3)cc2F)s1] |
| 177 | 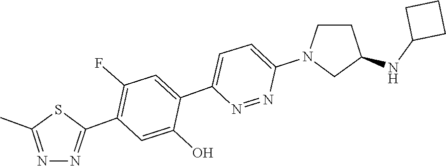 [SMILES: Cc1nnc(-c2cc(O)c(-c3ccc(N4CC[C@@H](NC5CCC5)C4)nn3)cc2F)s1] |
| 178 | 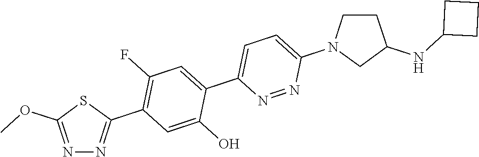 [SMILES: COc1nnc(-c2cc(O)c(-c3ccc(N4CCC(NC5CCC5)C4)nn3)cc2F)s1] |
| 179 | 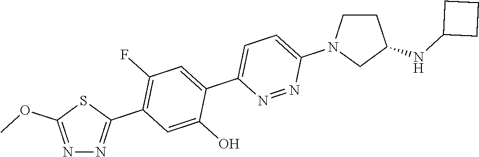 [SMILES: COc1nnc(-c2cc(O)c(-c3ccc(N4CC[C@H](NC5CCC5)C4)nn3)cc2F)s1] |
| 180 | 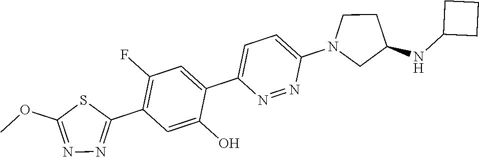 [SMILES: COc1nnc(-c2cc(O)c(-c3ccc(N4CC[C@@H](NC5CCC5)C4)nn3)cc2F)s1] |
| 181 | 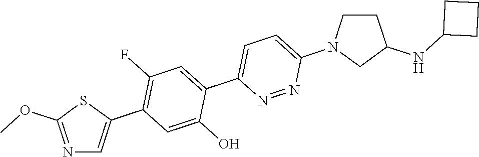 [SMILES: COc1ncc(-c2cc(O)c(-c3ccc(N4CCC(NC5CCC5)C4)nn3)cc2F)s1] |
| 182 | 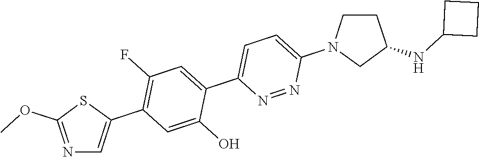 [SMILES: COc1ncc(-c2cc(O)c(-c3ccc(N4CC[C@H](NC5CCC5)C4)nn3)cc2F)s1] |
| 183 | 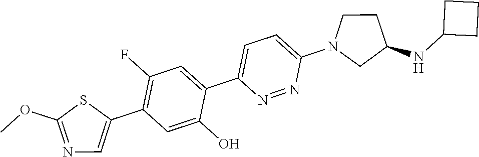 [SMILES: COc1ncc(-c2cc(O)c(-c3ccc(N4CC[C@@H](NC5CCC5)C4)nn3)cc2F)s1] |
| 184 | 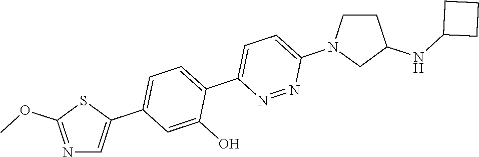 [SMILES: COc1ncc(-c2ccc(-c3ccc(N4CCC(NC5CCC5)C4)nn3)c(O)c2)s1] |
| 185 | 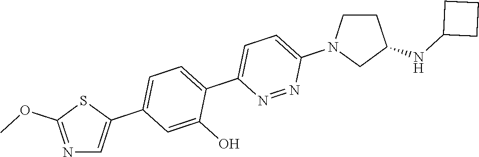 [SMILES: COc1ncc(-c2ccc(-c3ccc(N4CC[C@H](NC5CCC5)C4)nn3)c(O)c2)s1] |
| 186 | 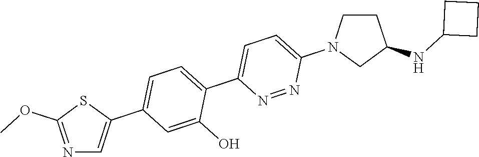 [SMILES: COc1ncc(-c2ccc(-c3ccc(N4CC[C@@H](NC5CCC5)C4)nn3)c(O)c2)s1] |
| 187 | 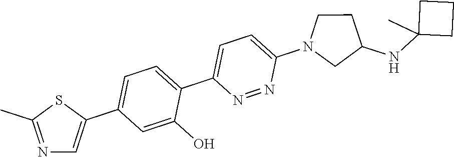 [SMILES: Cc1ncc(-c2ccc(-c3ccc(N4CCC(NC5(C)CCC5)C4)nn3)c(O)c2)s1] |
| 188 | 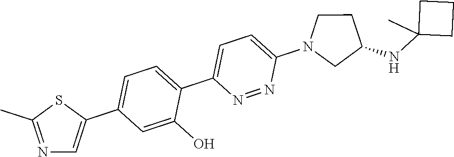 [SMILES: Cc1ncc(-c2ccc(-c3ccc(N4CC[C@H](NC5(C)CCC5)C4)nn3)c(O)c2)s1] |
| 189 | 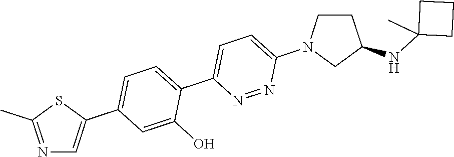 [SMILES: Cc1ncc(-c2ccc(-c3ccc(N4CC[C@@H](NC5(C)CCC5)C4)nn3)c(O)c2)s1] |
| 190 | 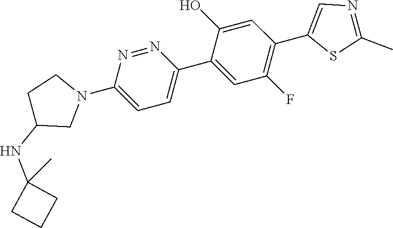 [SMILES: Cc1ncc(-c2cc(O)c(-c3ccc(N4CCC(NC5(C)CCC5)C4)nn3)cc2F)s1] |
| 191 | 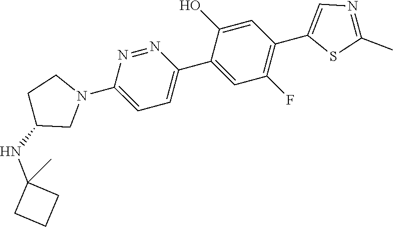 [SMILES: Cc1ncc(-c2cc(O)c(-c3ccc(N4CC[C@@H](NC5(C)CCC5)C4)nn3)cc2F)s1] |
| 192 | 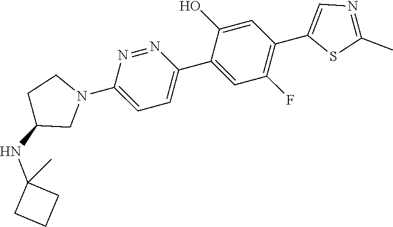 [SMILES: Cc1ncc(-c2cc(O)c(-c3ccc(N4CC[C@H](NC5(C)CCC5)C4)nn3)cc2F)s1] |
| 193 | 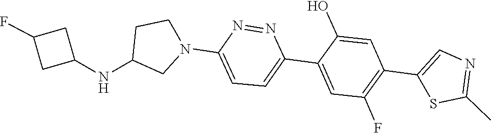 [SMILES: Cc1ncc(-c2cc(O)c(-c3ccc(N4CCC(NC5CC(F)C5)C4)nn3)cc2F)s1] |
| 194 | 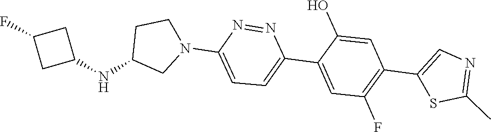 |
| 195 | 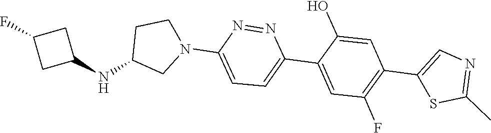 |
| 196 | 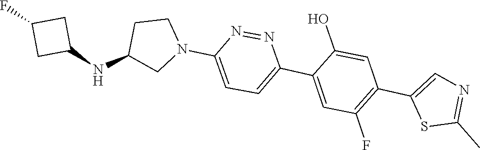 |
| 197 | 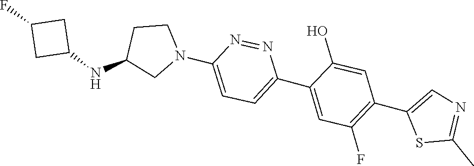 |
| 198 | 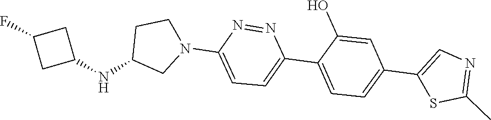 |
| 199 | 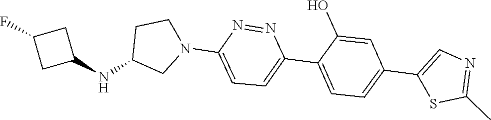 |
| 200 | 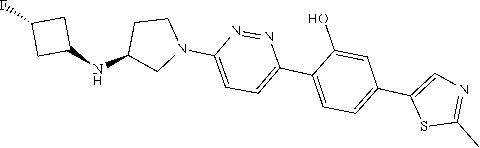 |
| 201 | 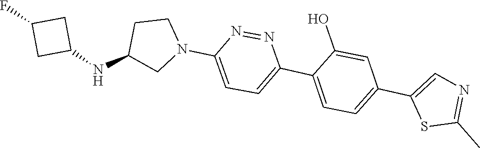 |
| 202 | 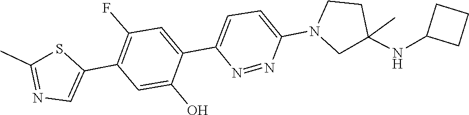 [SMILES: Cc1ncc(-c2cc(O)c(-c3ccc(N4CCC(C)(NC5CCC5)C4)nn3)cc2F)s1] |
| 203 | 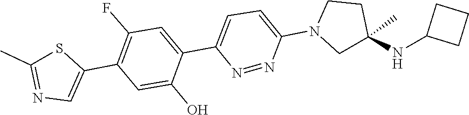 [SMILES: Cc1ncc(-c2cc(O)c(-c3ccc(N4CC[C@@](C)(NC5CCC5)C4)nn3)cc2F)s1] |
| 204 | 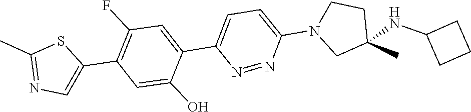 [SMILES: Cc1ncc(-c2cc(O)c(-c3ccc(N4CC[C@](C)(NC5CCC5)C4)nn3)cc2F)s1] |
| 205 | 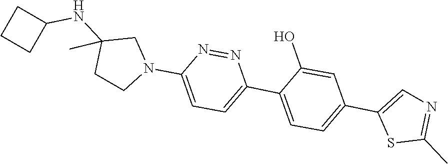 [SMILES: Cc1ncc(-c2ccc(-c3ccc(N4CCC(C)(NC5CCC5)C4)nn3)c(O)c2)s1] |
| 206 | 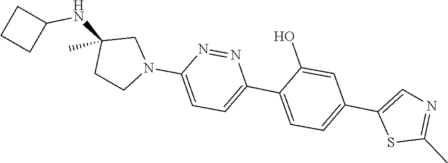 [SMILES: Cc1ncc(-c2ccc(-c3ccc(N4CC[C@@](C)(NC5CCC5)C4)nn3)c(O)c2)s1] |
| 207 | 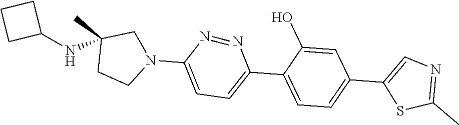 [SMILES: Cc1ncc(-c2ccc(-c3ccc(N4CC[C@](C)(NC5CCC5)C4)nn3)c(O)c2)s1] |
| 208 | 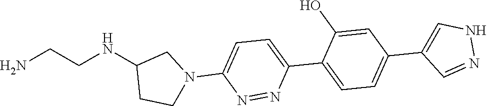 [SMILES: NCCNC1CCN(c2ccc(-c3ccc(-c4cn[nH]c4)cc3O)nn2)C1] |
| 209 | 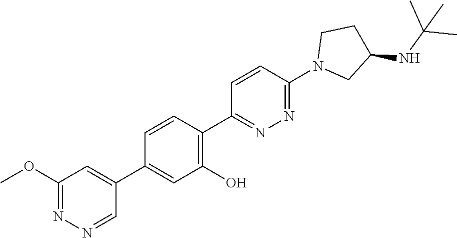 [SMILES: COc1cc(-c2ccc(-c3ccc(N4CC[C@@H](NC(C)(C)C)C4)nn3)c(O)c2)cnn1] |
| 210 | 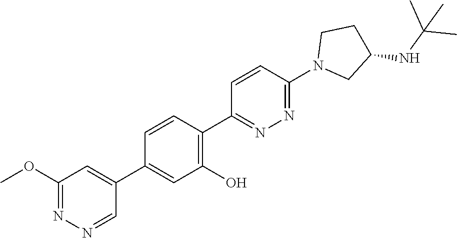 [SMILES: COc1cc(-c2ccc(-c3ccc(N4CC[C@H](NC(C)(C)C)C4)nn3)c(O)c2)cnn1] |
| 211 | 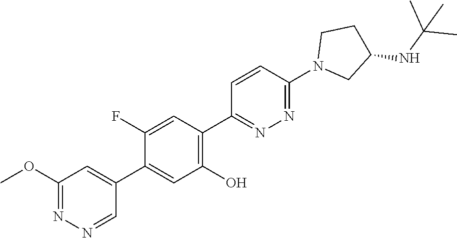 [SMILES: COc1cc(-c2cc(O)c(-c3ccc(N4CC[C@H](NC(C)(C)C)C4)nn3)cc2F)cnn1] |
| 212 | 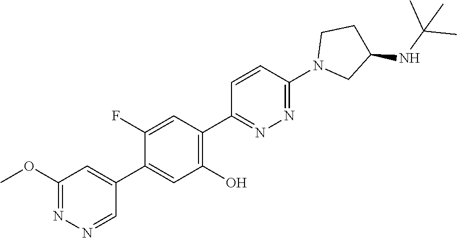 [SMILES: COc1cc(-c2cc(O)c(-c3ccc(N4CC[C@@H](NC(C)(C)C)C4)nn3)cc2F)cnn1] |
| 213 | 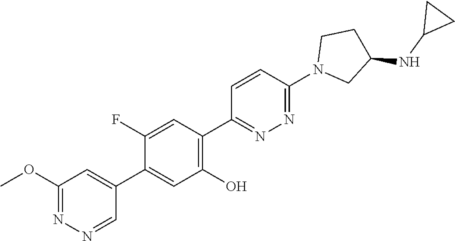 [SMILES: COc1cc(-c2cc(O)c(-c3ccc(N4CC[C@@H](NC5CC5)C4)nn3)cc2F)cnn1] |
| 214 | 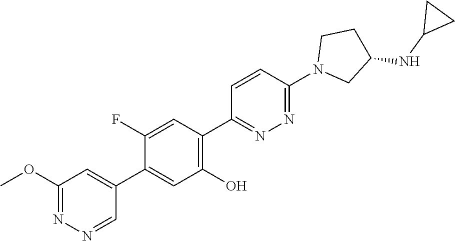 [SMILES: COc1cc(-c2cc(O)c(-c3ccc(N4CC[C@H](NC5CC5)C4)nn3)cc2F)cnn1] |
| 215 | 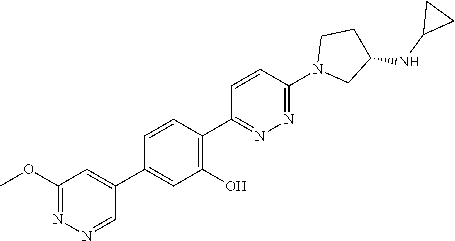 [SMILES: COc1cc(-c2ccc(-c3ccc(N4CC[C@H](NC5CC5)C4)nn3)c(O)c2)cnn1] |
| 216 | 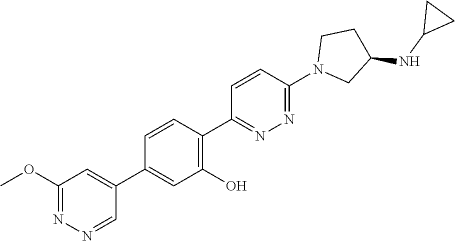 [SMILES: COc1cc(-c2ccc(-c3ccc(N4CC[C@@H](NC5CC5)C4)nn3)c(O)c2)cnn1] |
| 217 | 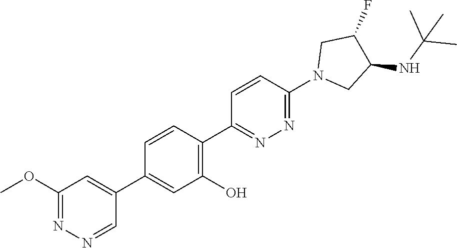 [SMILES: COc1cc(-c2ccc(-c3ccc(N4C[C@H](NC(C)(C)C)[C@@H](F)C4)nn3)c(O)c2)cnn1] |
| 218 | 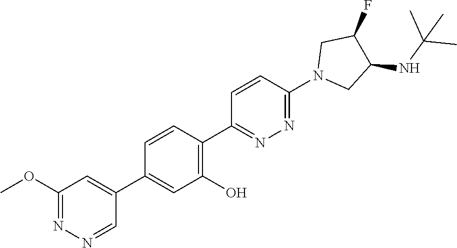 [SMILES: COc1cc(-c2ccc(-c3ccc(N4C[C@@H](F)[C@@H](NC(C)(C)C)C4)nn3)c(O)c2)cnn1] |
| 219 | 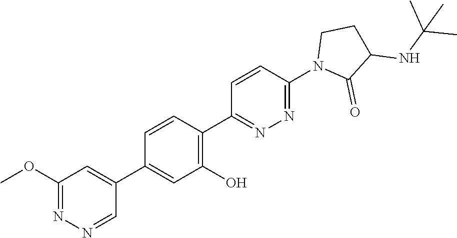 [SMILES: COc1cc(-c2ccc(-c3ccc(N4CCC(NC(C)(C)C)C4=O)nn3)c(O)c2)cnn1] |
| 220 | 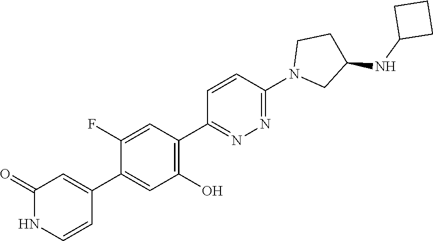 [SMILES: O=c1cc(-c2cc(O)c(-c3ccc(N4CC[C@@H](NC5CCC5)C4)nn3)cc2F)cc[nH]1] |
| 221 | 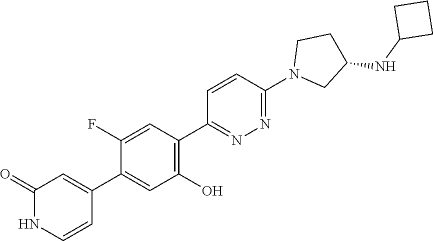 [SMILES: O=c1cc(-c2cc(O)c(-c3ccc(N4CC[C@H](NC5CCC5)C4)nn3)cc2F)cc[nH]1] |
| 222 | 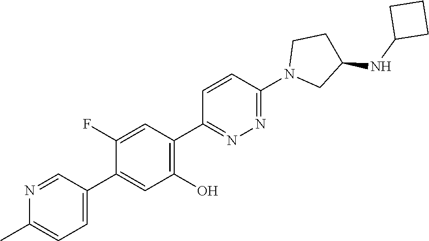 [SMILES: Cc1ccc(-c2cc(O)c(-c3ccc(N4CC[C@@H](NC5CCC5)C4)nn3)cc2F)cn1] |
| 223 | 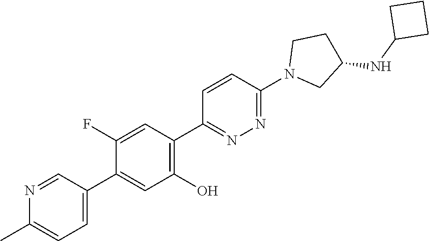 [SMILES: Cc1ccc(-c2cc(O)c(-c3ccc(N4CC[C@H](NC5CCC5)C4)nn3)cc2F)cn1] |
| 224 | 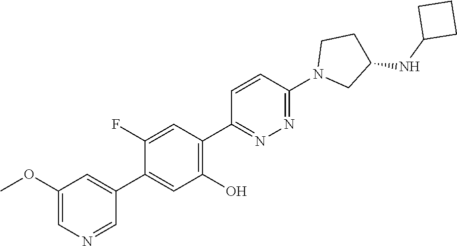 [SMILES: COc1cncc(-c2cc(O)c(-c3ccc(N4CC[C@H](NC5CCC5)C4)nn3)cc2F)c1] |
| 225 | 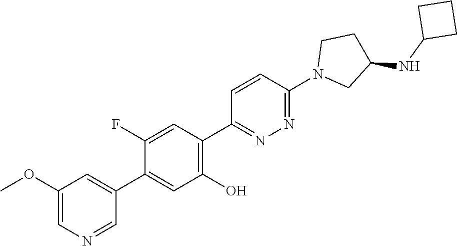 [SMILES: COc1cncc(-c2cc(O)c(-c3ccc(N4CC[C@@H](NC5CCC5)C4)nn3)cc2F)c1] |
| 226 | 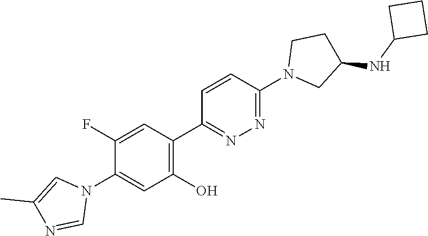 [SMILES: Cc1cn(-c2cc(O)c(-c3ccc(N4CC[C@@H](NC5CCC5)C4)nn3)cc2F)cn1] |
| 227 | 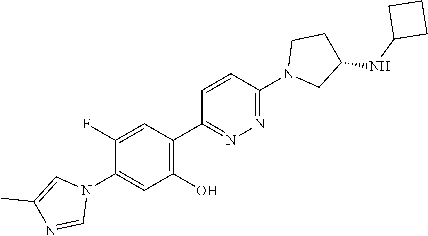 [SMILES: Cc1cn(-c2cc(O)c(-c3ccc(N4CC[C@H](NC5CCC5)C4)nn3)cc2F)cn1] |
| 228 | 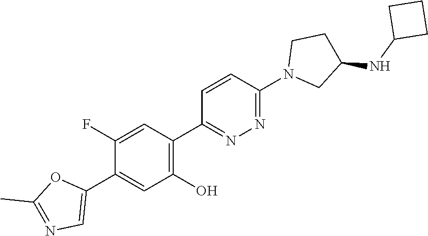 [SMILES: Cc1ncc(-c2cc(O)c(-c3ccc(N4CC[C@@H](NC5CCC5)C4)nn3)cc2F)o1] |
| 229 | 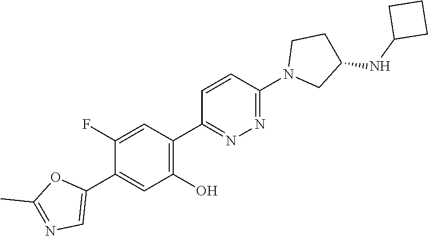 [SMILES: Cc1ncc(-c2cc(O)c(-c3ccc(N4CC[C@H](NC5CCC5)C4)nn3)cc2F)o1] |
| 230 | 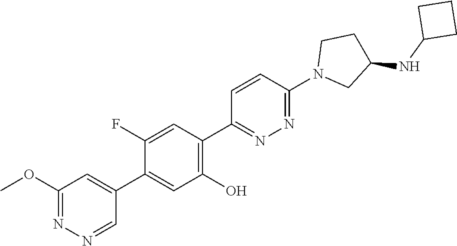 [SMILES: COc1cc(-c2cc(O)c(-c3ccc(N4CC[C@@H](NC5CCC5)C4)nn3)cc2F)cnn1] |
| 231 | 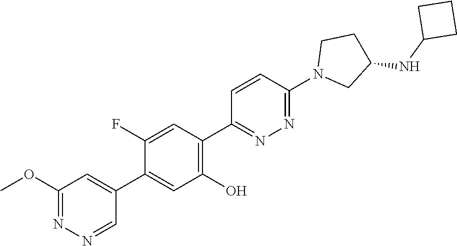 [SMILES: COc1cc(-c2cc(O)c(-c3ccc(N4CC[C@H](NC5CCC5)C4)nn3)cc2F)cnn1] |
| 232 | 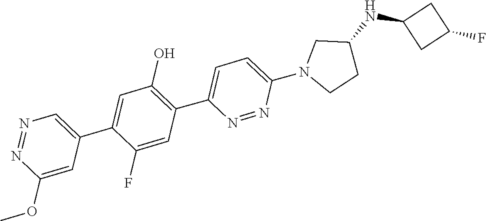 [SMILES: COc1cc(-c2cc(O)c(-c3ccc(N4CC[C@@H](N[C@H]5C[C@H](F)C5)C4)nn3)cc2F)cnn1] |
| 234 | 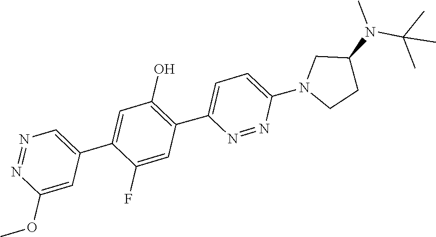 [SMILES: COc1cc(-c2cc(O)c(-c3ccc(N4CC[C@H](N(C)C(C)(C)C)C4)nn3)cc2F)cnn1] |
| 235 | 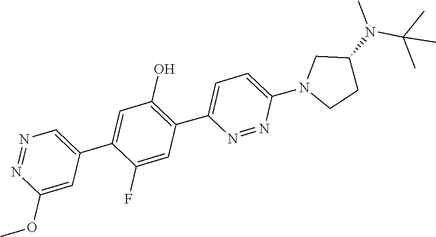 [SMILES: COc1cc(-c2cc(O)c(-c3ccc(N4CC[C@@H](N(C)C(C)(C)C)C4)nn3)cc2F)cnn1] |
| 236 | 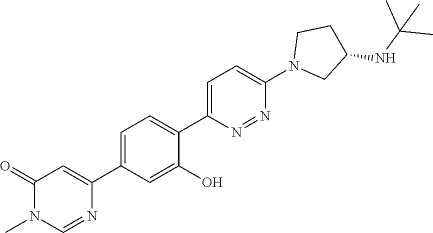 [SMILES: Cn1cnc(-c2ccc(-c3ccc(N4CC[C@H](NC(C)(C)C)C4)nn3)c(O)c2)cc1=O] |
| 237 | 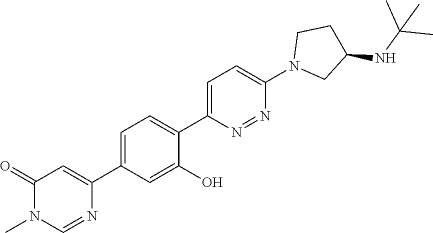 [SMILES: Cn1cnc(-c2ccc(-c3ccc(N4CC[C@@H](NC(C)(C)C)C4)nn3)c(O)c2)cc1=O] |
| 238 | 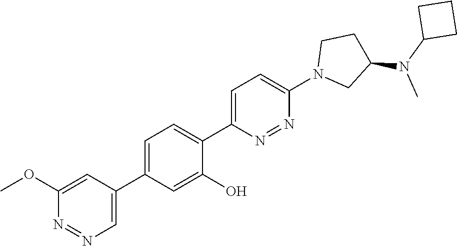 [SMILES: COc1cc(-c2ccc(-c3ccc(N4CC[C@@H](N(C)C5CCC5)C4)nn3)c(O)c2)cnn1] |
| 239 | 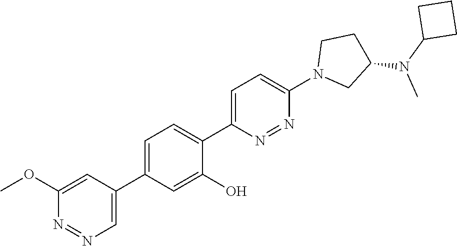 [SMILES: COc1cc(-c2ccc(-c3ccc(N4CC[C@H](N(C)C5CCC5)C4)nn3)c(O)c2)cnn1] |
| 241 | 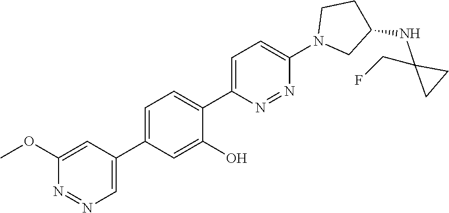 [SMILES: COc1cc(-c2ccc(-c3ccc(N4CC[C@H](NC5(CF)CC5)C4)nn3)c(O)c2)cnn1] |
| 242 | 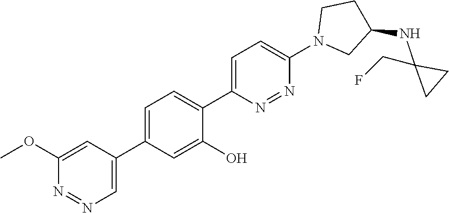 [SMILES: COc1cc(-c2ccc(-c3ccc(N4CC[C@@H](NC5(CF)CC5)C4)nn3)c(O)c2)cnn1] |
| 243 | 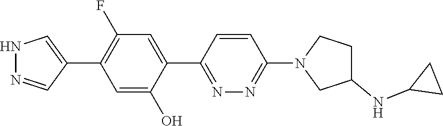 [SMILES: Oc1cc(-c2cn[nH]c2)c(F)cc1-c1ccc(N2CCC(NC3CC3)C2)nn1] |
| 244 | 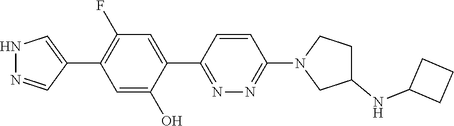 [SMILES: Oc1cc(-c2cn[nH]c2)c(F)cc1-c1ccc(N2CCC(NC3CCC3)C2)nn1] |
| 245 | 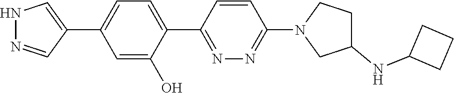 [SMILES: Oc1cc(-c2cn[nH]c2)ccc1-c1ccc(N2CCC(NC3CCC3)C2)nn1] |
| 246 | 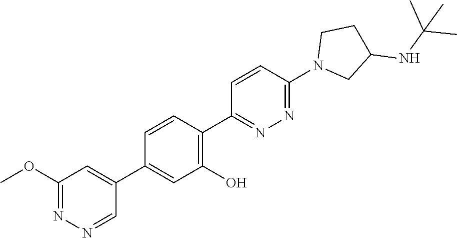 [SMILES: COc1cc(-c2ccc(-c3ccc(N4CCC(NC(C)(C)C)C4)nn3)c(O)c2)cnn1] |
| 247 | 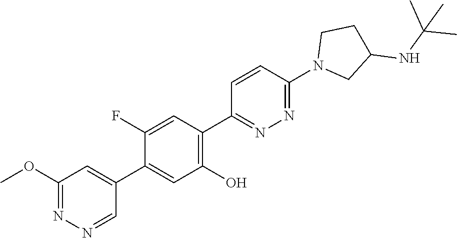 [SMILES: COc1cc(-c2cc(O)c(-c3ccc(N4CCC(NC(C)(C)C)C4)nn3)cc2F)cnn1] |
| 248 | 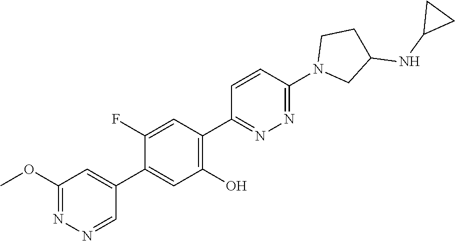 [SMILES: COc1cc(-c2cc(O)c(-c3ccc(N4CCC(NC5CC5)C4)nn3)cc2F)cnn1] |
| 249 | 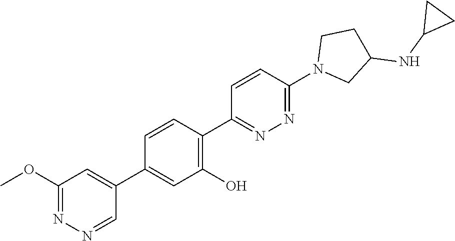 [SMILES: COc1cc(-c2ccc(-c3ccc(N4CCC(NC5CC5)C4)nn3)c(O)c2)cnn1] |
| 250 | 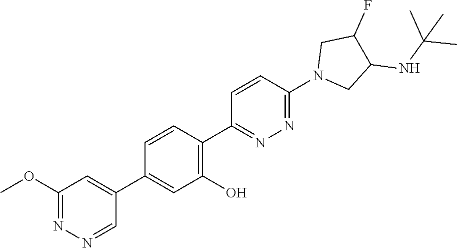 [SMILES: COc1cc(-c2ccc(-c3ccc(N4CC(F)C(NC(C)(C)C)C4)nn3)c(O)c2)cnn1] |
| 251 | 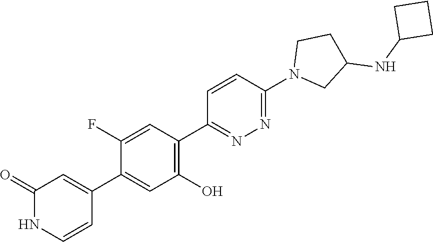 [SMILES: O=c1cc(-c2cc(O)c(-c3ccc(N4CCC(NC5CCC5)C4)nn3)cc2F)cc[nH]1] |
| 252 | 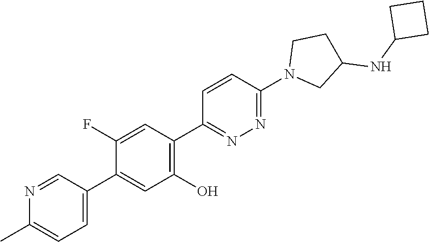 [SMILES: Cc1ccc(-c2cc(O)c(-c3ccc(N4CCC(NC5CCC5)C4)nn3)cc2F)cn1] |
| 253 | 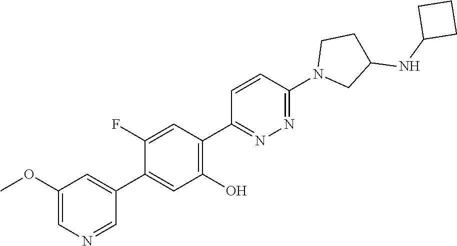 [SMILES: COc1cncc(-c2cc(O)c(-c3ccc(N4CCC(NC5CCC5)C4)nn3)cc2F)c1] |
| 254 | 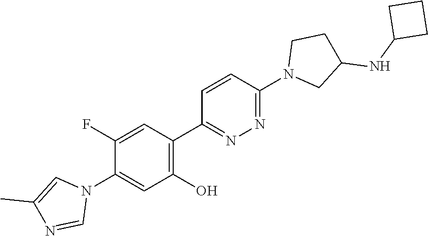 [SMILES: Cc1cn(-c2cc(O)c(-c3ccc(N4CCC(NC5CCC5)C4)nn3)cc2F)cn1] |
| 255 | 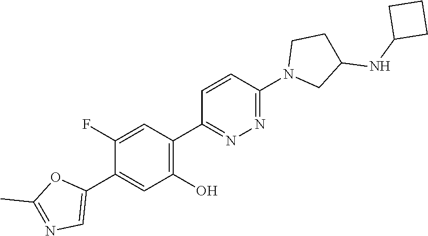 [SMILES: Cc1ncc(-c2cc(O)c(-c3ccc(N4CCC(NC5CCC5)C4)nn3)cc2F)o1] |
| 256 | 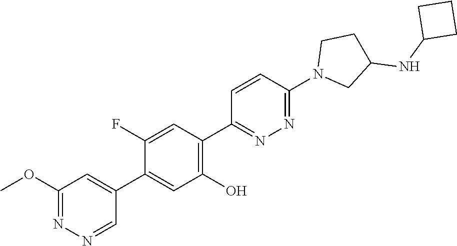 [SMILES: COc1cc(-c2cc(O)c(-c3ccc(N4CCC(NC5CCC5)C4)nn3)cc2F)cnn1] |
| 257 | 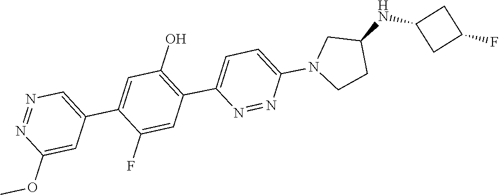 [SMILES: COc1cc(-c2cc(O)c(-c3ccc(N4CC[C@H](N[C@H]5C[C@@H](F)C5)C4)nn3)cc2F)cnn1] |
| 258 | 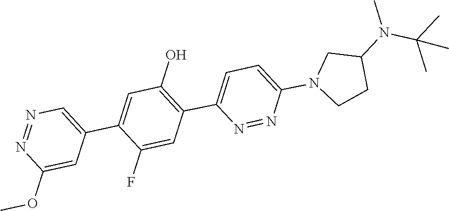 [SMILES: COc1cc(-c2cc(O)c(-c3ccc(N4CCC(N(C)C(C)(C)C)C4)nn3)cc2F)cnn1] |
| 259 | 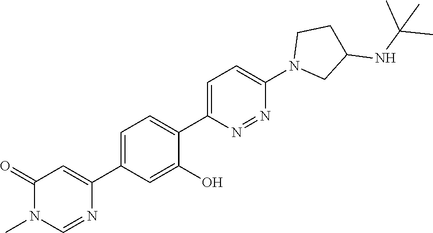 [SMILES: Cn1cnc(-c2ccc(-c3ccc(N4CCC(NC(C)(C)C)C4)nn3)c(O)c2)cc1=O] |
| 260 | 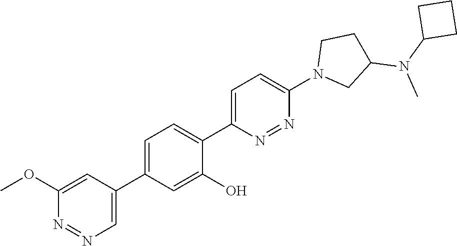 [SMILES: COc1cc(-c2ccc(-c3ccc(N4CCC(N(C)C5CCC5)C4)nn3)c(O)c2)cnn1] |
| 261 | 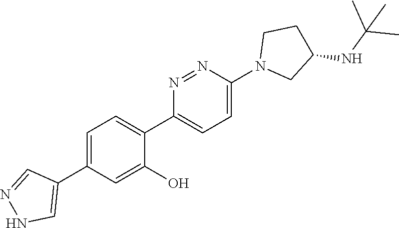 [SMILES: CC(C)(C)N[C@H]1CCN(c2ccc(-c3ccc(-c4cn[nH]c4)cc3O)nn2)C1] |
| 262 | 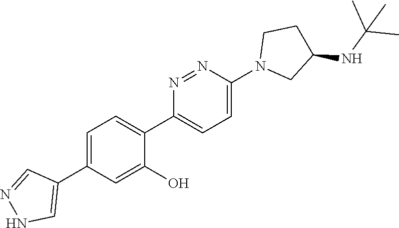 [SMILES: CC(C)(C)N[C@@H]1CCN(c2ccc(-c3ccc(-c4cn[nH]c4)cc3O)nn2)C1] |
| 263 | 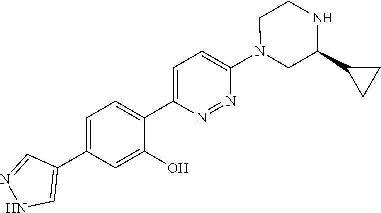 [SMILES: Oc1cc(-c2cn[nH]c2)ccc1-c1ccc(N2CCN[C@@H](C3CC3)C2)nn1] |
| 264 | 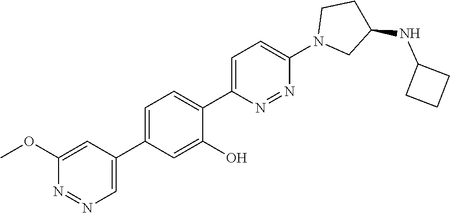 [SMILES: COc1cc(-c2ccc(-c3ccc(N4CC[C@@H](NC5CCC5)C4)nn3)c(O)c2)cnn1] |
| 267 | 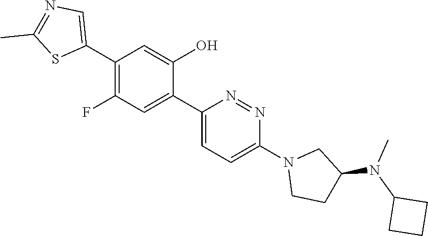 [SMILES: Cc1ncc(-c2cc(O)c(-c3ccc(N4CC[C@H](N(C)C5CCC5)C4)nn3)cc2F)s1] |
| 268 | 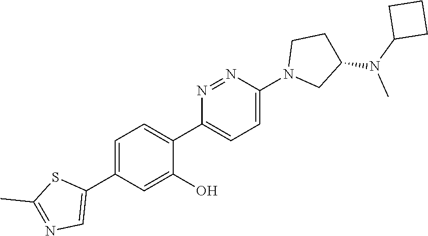 [SMILES: Cc1ncc(-c2ccc(-c3ccc(N4CC[C@H](N(C)C5CCC5)C4)nn3)c(O)c2)s1] |
| 269 | 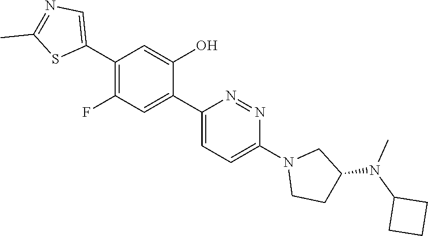 [SMILES: Cc1ncc(-c2cc(O)c(-c3ccc(N4CC[C@@H](N(C)C5CCC5)C4)nn3)cc2F)s1] |
| 270 | 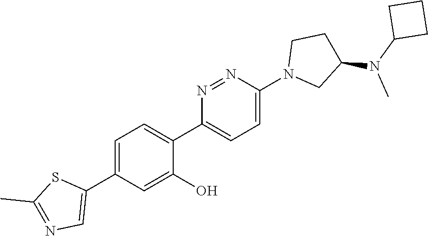 [SMILES: Cc1ncc(-c2ccc(-c3ccc(N4CC[C@@H](N(C)C5CCC5)C4)nn3)c(O)c2)s1] |
| 271 | 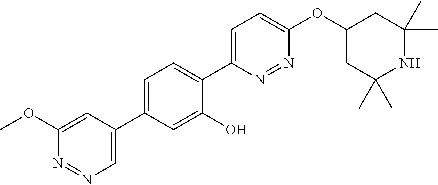 |
| 272 | 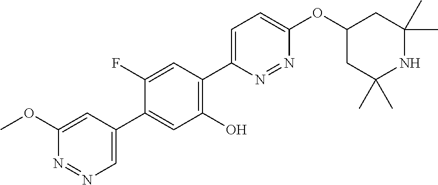 |
| 273 | 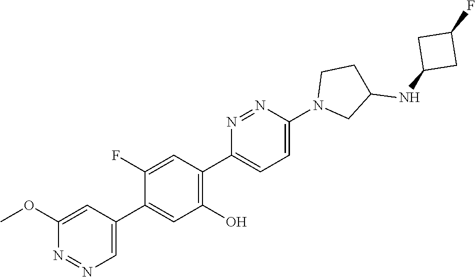 [SMILES: COc1cc(-c2cc(O)c(-c3ccc(N4CCC(N[C@H]5C[C@@H](F)C5)C4)nn3)cc2F)cnn1] |
| 274 | 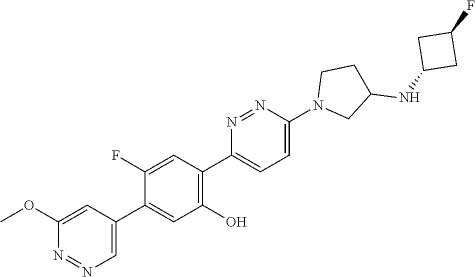 [SMILES: COc1cc(-c2cc(O)c(-c3ccc(N4CCC(N[C@H]5C[C@H](F)C5)C4)nn3)cc2F)cnn1] |
| 275 | 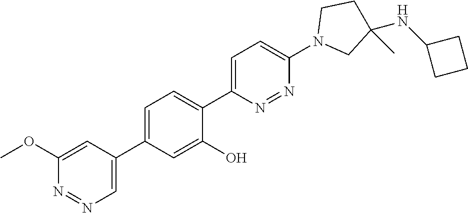 [SMILES: COc1cc(-c2ccc(-c3ccc(N4CCC(C)(NC5CCC5)C4)nn3)c(O)c2)cnn1] |
| 276 | 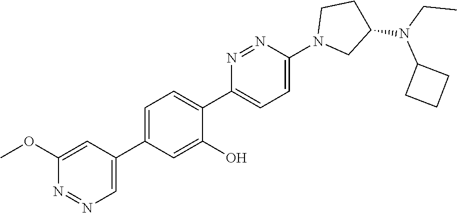 [SMILES: CCN(C1CCC1)[C@H]1CCN(c2ccc(-c3ccc(-c4cnnc(OC)c4)cc3O)nn2)C1] |
| 277 | 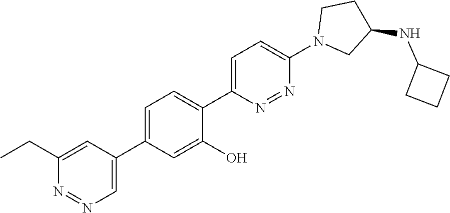 [SMILES: CCc1cc(-c2ccc(-c3ccc(N4CC[C@@H](NC5CCC5)C4)nn3)c(O)c2)cnn1] |
| 278 | 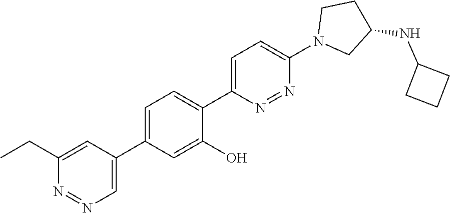 [SMILES: CCc1cc(-c2ccc(-c3ccc(N4CC[C@H](NC5CCC5)C4)nn3)c(O)c2)cnn1] |
| 279 | 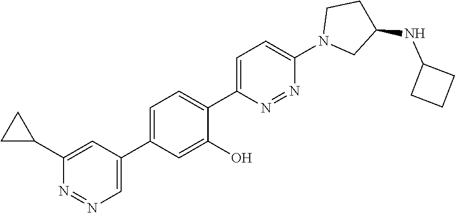 [SMILES: Oc1cc(-c2cnnc(C3CC3)c2)ccc1-c1ccc(N2CC[C@@H](NC3CCC3)C2)nn1] |
| 280 | 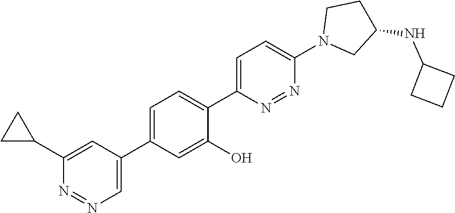 [SMILES: Oc1cc(-c2cnnc(C3CC3)c2)ccc1-c1ccc(N2CC[C@H](NC3CCC3)C2)nn1] |
| 281 | 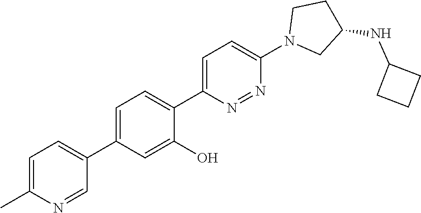 [SMILES: Cc1ccc(-c2ccc(-c3ccc(N4CC[C@H](NC5CCC5)C4)nn3)c(O)c2)cn1] |
| 282 | 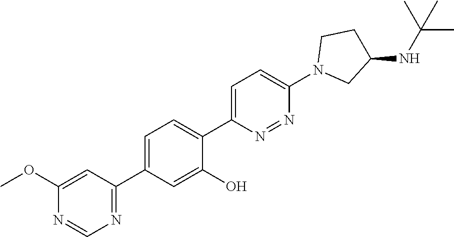 [SMILES: COc1cc(-c2ccc(-c3ccc(N4CC[C@@H](NC(C)(C)C)C4)nn3)c(O)c2)ncn1] |
| 283 | 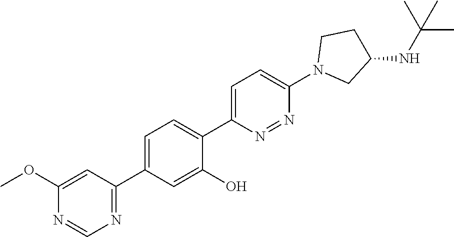 [SMILES: COc1cc(-c2ccc(-c3ccc(N4CC[C@H](NC(C)(C)C)C4)nn3)c(O)c2)ncn1] |
| 284 | 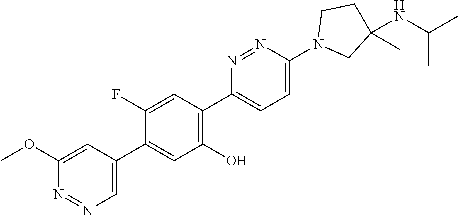 [SMILES: COc1cc(-c2cc(O)c(-c3ccc(N4CCC(C)(NC(C)C)C4)nn3)cc2F)cnn1] |
| 285 | 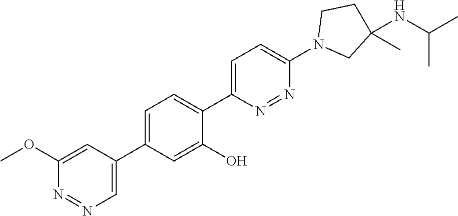 [SMILES: COc1cc(-c2ccc(-c3ccc(N4CCC(C)(NC(C)C)C4)nn3)c(O)c2)cnn1] |
| 286 | 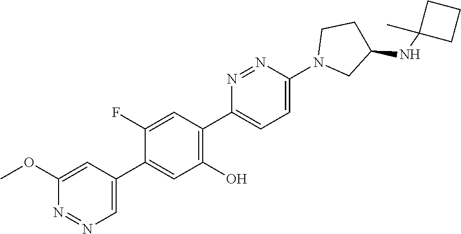 [SMILES: COc1cc(-c2cc(O)c(-c3ccc(N4CC[C@@H](NC5(C)CCC5)C4)nn3)cc2F)cnn1] |
| 287 | 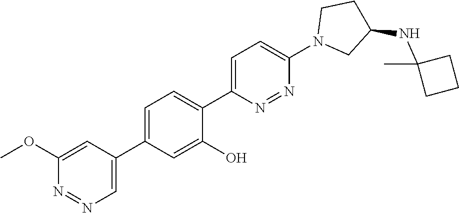 [SMILES: COc1cc(-c2ccc(-c3ccc(N4CC[C@@H](NC5(C)CCC5)C4)nn3)c(O)c2)cnn1] |
| 288 | 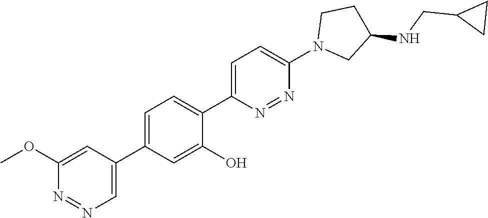 [SMILES: COc1cc(-c2ccc(-c3ccc(N4CC[C@@H](NCC5CC5)C4)nn3)c(O)c2)cnn1] |
| 289 | 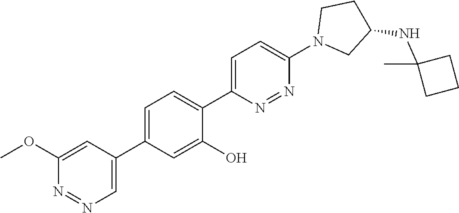 [SMILES: COc1cc(-c2ccc(-c3ccc(N4CC[C@H](NC5(C)CCC5)C4)nn3)c(O)c2)cnn1] |
| 290 | 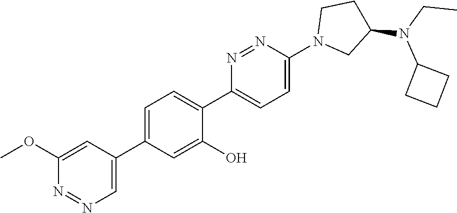 [SMILES: CCN(C1CCC1)[C@@H]1CCN(c2ccc(-c3ccc(-c4cnnc(OC)c4)cc3O)nn2)C1] |
| 291 | 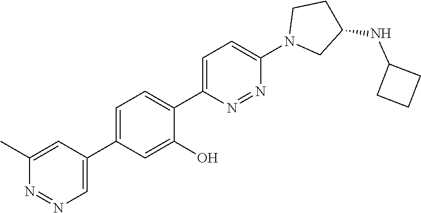 [SMILES: Cc1cc(-c2ccc(-c3ccc(N4CC[C@H](NC5CCC5)C4)nn3)c(O)c2)cnn1] |
| 292 | 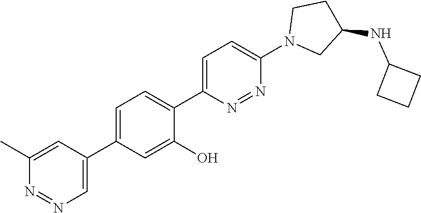 [SMILES: Cc1cc(-c2ccc(-c3ccc(N4CC[C@@H](NC5CCC5)C4)nn3)c(O)c2)cnn1] |
| 293 | 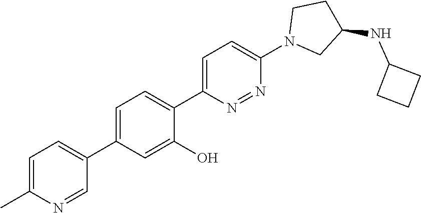 [SMILES: Cc1ccc(-c2ccc(-c3ccc(N4CC[C@@H](NC5CCC5)C4)nn3)c(O)c2)cn1] |
| 294 | 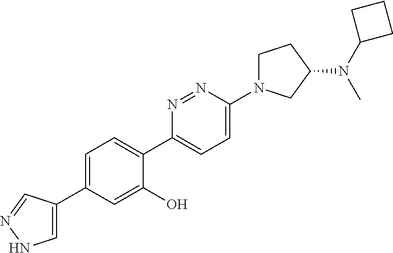 [SMILES: CN(C1CCC1)[C@H]1CCN(c2ccc(-c3ccc(-c4cn[nH]c4)cc3O)nn2)C1] |
| 295 | 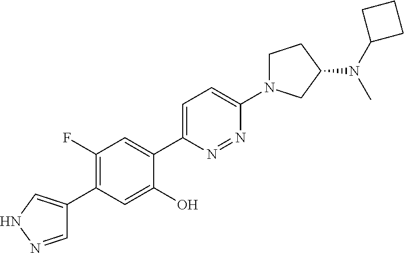 [SMILES: CN(C1CCC1)[C@H]1CCN(c2ccc(-c3cc(F)c(-c4cn[nH]c4)cc3O)nn2)C1] |
| 296 | 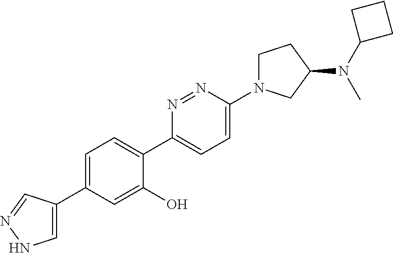 [SMILES: CN(C1CCC1)[C@@H]1CCN(c2ccc(-c3ccc(-c4cn[nH]c4)cc3O)nn2)C1] |
| 297 | 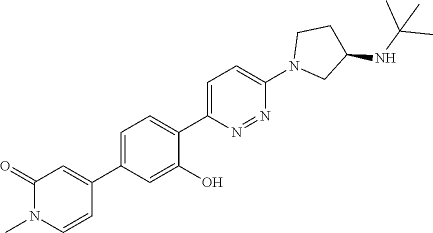 [SMILES: Cn1ccc(-c2ccc(-c3ccc(N4CC[C@@H](NC(C)(C)C)C4)nn3)c(O)c2)cc1=O] |
| 298 | 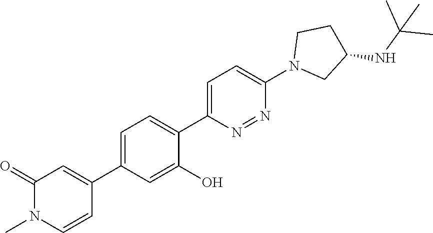 [SMILES: Cn1ccc(-c2ccc(-c3ccc(N4CC[C@H](NC(C)(C)C)C4)nn3)c(O)c2)cc1=O] |
| 299 | 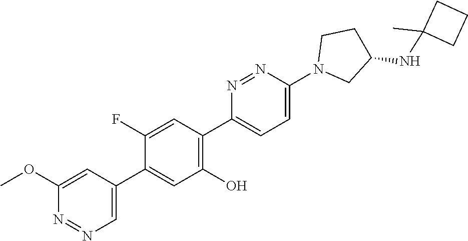 [SMILES: COc1cc(-c2cc(O)c(-c3ccc(N4CC[C@H](NC5(C)CCC5)C4)nn3)cc2F)cnn1] |
| 301 | 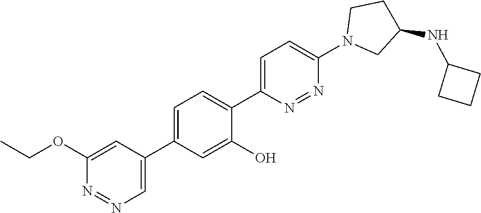 [SMILES: CCOc1cc(-c2ccc(-c3ccc(N4CC[C@@H](NC5CCC5)C4)nn3)c(O)c2)cnn1] |
| 302 | 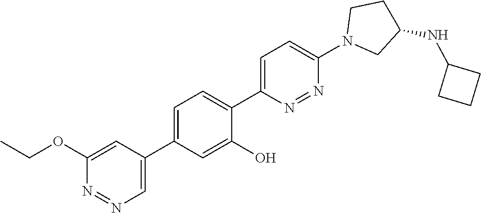 [SMILES: CCOc1cc(-c2ccc(-c3ccc(N4CC[C@H](NC5CCC5)C4)nn3)c(O)c2)cnn1] |
| 303 | 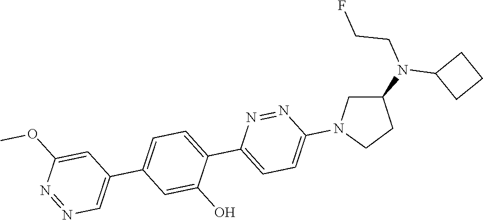 [SMILES: COc1cc(-c2ccc(-c3ccc(N4CC[C@H](N(CCF)C5CCC5)C4)nn3)c(O)c2)cnn1] |
| 304 | 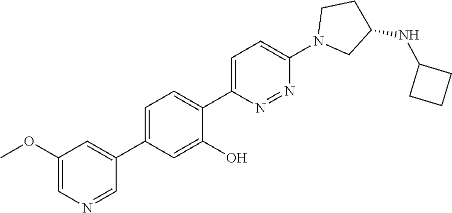 [SMILES: COc1cncc(-c2ccc(-c3ccc(N4CC[C@H](NC5CCC5)C4)nn3)c(O)c2)c1] |
| 305 | 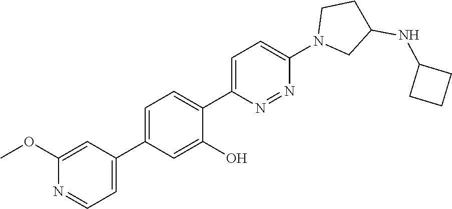 [SMILES: COc1cc(-c2ccc(-c3ccc(N4CCC(NC5CCC5)C4)nn3)c(O)c2)ccn1] |
| 306 | 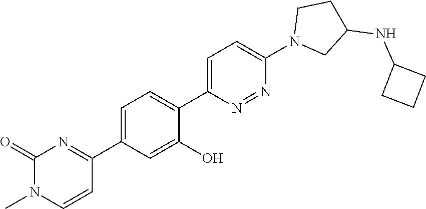 [SMILES: Cn1ccc(-c2ccc(-c3ccc(N4CCC(NC5CCC5)C4)nn3)c(O)c2)nc1=O] |
| 307 | 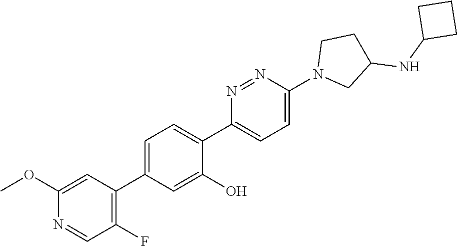 [SMILES: COc1cc(-c2ccc(-c3ccc(N4CCC(NC5CCC5)C4)nn3)c(O)c2)c(F)cn1] |
| 308 | 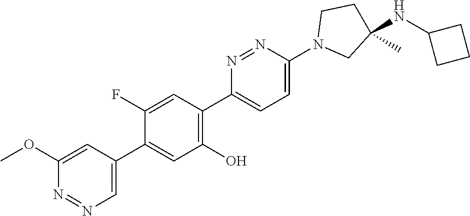 [SMILES: COc1cc(-c2cc(O)c(-c3ccc(N4CC[C@@](C)(NC5CCC5)C4)nn3)cc2F)cnn1] |
| 309 | 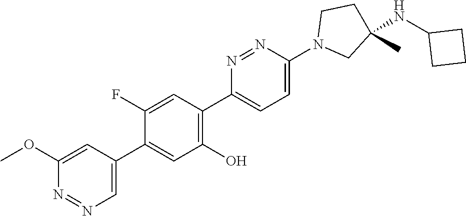 [SMILES: COc1cc(-c2cc(O)c(-c3ccc(N4CC[C@](C)(NC5CCC5)C4)nn3)cc2F)cnn1] |
| 310 | 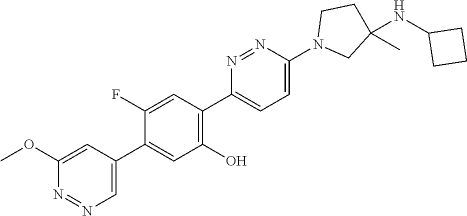 [SMILES: COc1cc(-c2cc(O)c(-c3ccc(N4CCC(C)(NC5CCC5)C4)nn3)cc2F)cnn1] |
| 311 | 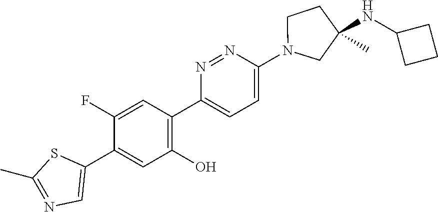 [SMILES: Cc1ncc(-c2cc(O)c(-c3ccc(N4CC[C@@](C)(NC5CCC5)C4)nn3)cc2F)s1] |
| 312 | 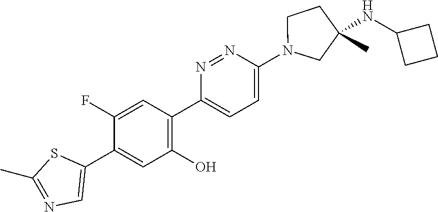 [SMILES: Cc1ncc(-c2cc(O)c(-c3ccc(N4CC[C@](C)(NC5CCC5)C4)nn3)cc2F)s1] |
| 313 | 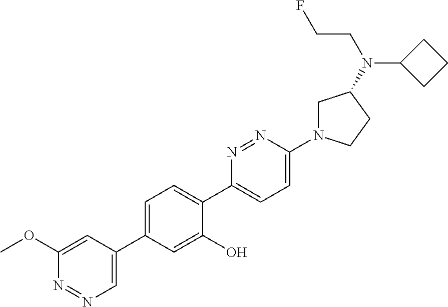 [SMILES: COc1cc(-c2ccc(-c3ccc(N4CC[C@@H](N(CCF)C5CCC5)C4)nn3)c(O)c2)cnn1] |
| 314 | 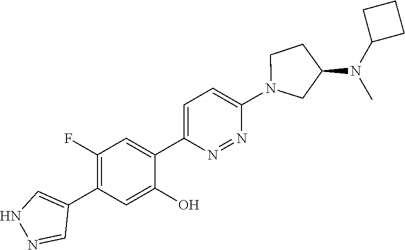 [SMILES: CN(C1CCC1)[C@@H]1CCN(c2ccc(-c3cc(F)c(-c4cn[nH]c4)cc3O)nn2)C1] |
| 315 | 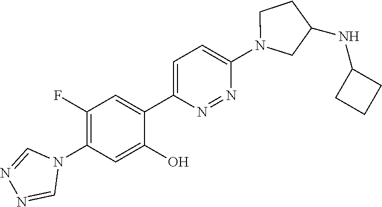 [SMILES: Oc1cc(-n2cnnc2)c(F)cc1-c1ccc(N2CCC(NC3CCC3)C2)nn1] |
| 316 | 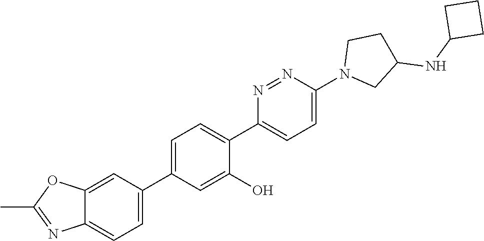 [SMILES: Cc1nc2ccc(-c3ccc(-c4ccc(N5CCC(NC6CCC6)C5)nn4)c(O)c3)cc2o1] |
| 317 | 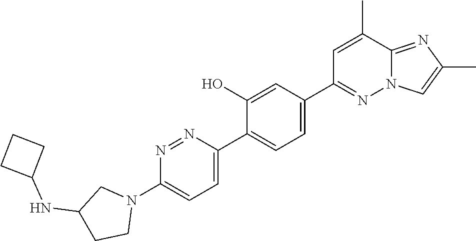 [SMILES: Cc1cn2nc(-c3ccc(-c4ccc(N5CCC(NC6CCC6)C5)nn4)c(O)c3)cc(C)c2n1] |
| 318 | 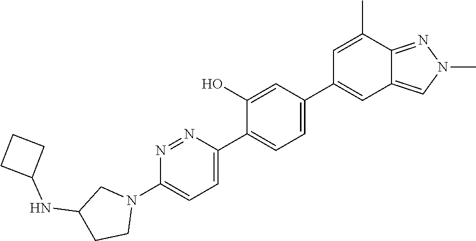 [SMILES: Cc1cc(-c2ccc(-c3ccc(N4CCC(NC5CCC5)C4)nn3)c(O)c2)cc2cn(C)nc12] |
| 319 | 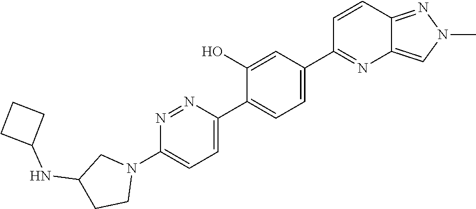 [SMILES: Cn1cc2nc(-c3ccc(-c4ccc(N5CCC(NC6CCC6)C5)nn4)c(O)c3)ccc2n1] |
| 320 | 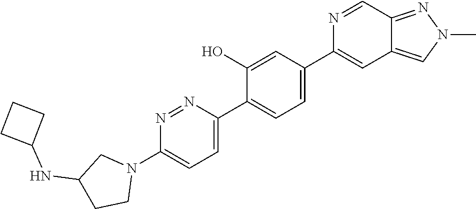 [SMILES: Cn1cc2cc(-c3ccc(-c4ccc(N5CCC(NC6CCC6)C5)nn4)c(O)c3)ncc2n1] |
| 321 | 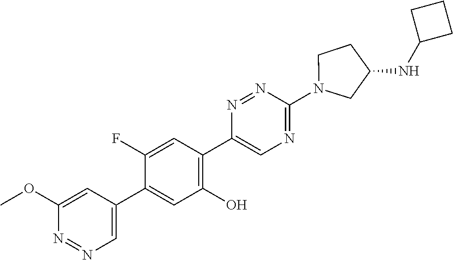 [SMILES: COc1cc(-c2cc(O)c(-c3cnc(N4CC[C@H](NC5CCC5)C4)nn3)cc2F)cnn1] |
| 322 | 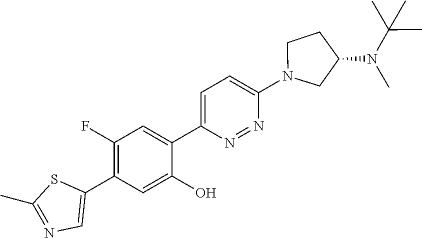 [SMILES: Cc1ncc(-c2cc(O)c(-c3ccc(N4CC[C@H](N(C)C(C)(C)C)C4)nn3)cc2F)s1] |
| 325 | 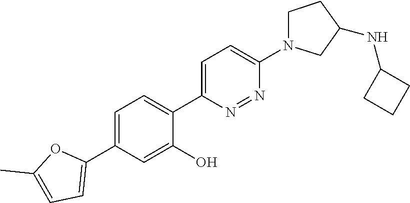 [SMILES: Cc1ccc(-c2ccc(-c3ccc(N4CCC(NC5CCC5)C4)nn3)c(O)c2)o1] |
| 326 | 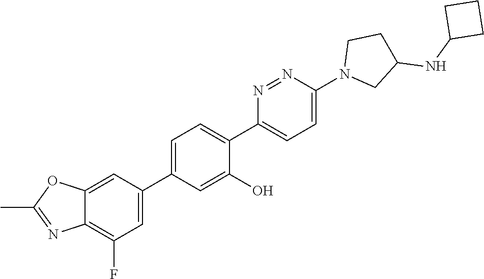 [SMILES: Cc1nc2c(F)cc(-c3ccc(-c4ccc(N5CCC(NC6CCC6)C5)nn4)c(O)c3)cc2o1] |
| 327 | 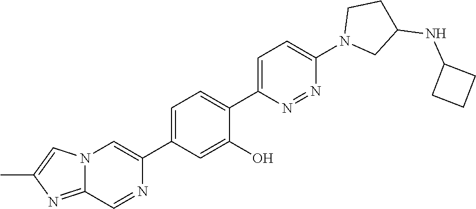 [SMILES: Cc1cn2cc(-c3ccc(-c4ccc(N5CCC(NC6CCC6)C5)nn4)c(O)c3)ncc2n1] |
| 328 | 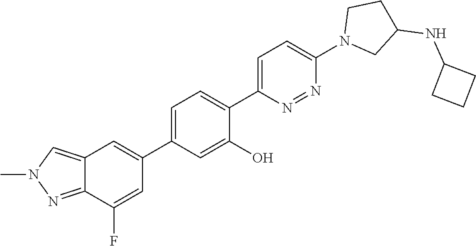 [SMILES: Cn1cc2cc(-c3ccc(-c4ccc(N5CCC(NC6CCC6)C5)nn4)c(O)c3)cc(F)c2n1] |
| 329 | 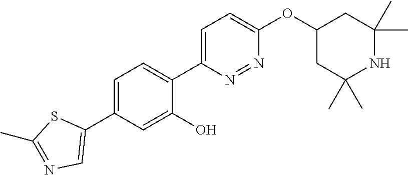 |
| 330 | 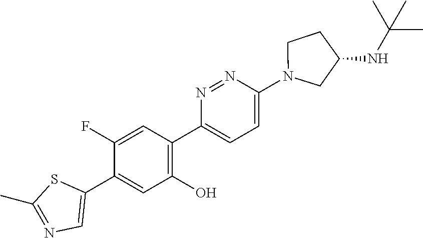 [SMILES: Cc1ncc(-c2cc(O)c(-c3ccc(N4CC[C@H](NC(C)(C)C)C4)nn3)cc2F)s1] |
| 331 | 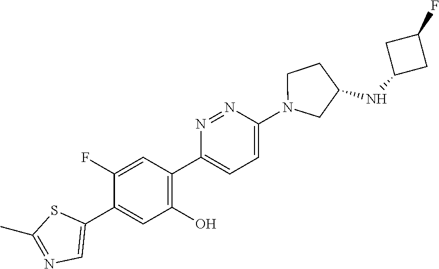 [SMILES: Cc1ncc(-c2cc(O)c(-c3ccc(N4CC[C@H](N[C@H]5C[C@H](F)C5)C4)nn3)cc2F)s1] |
| 332 | 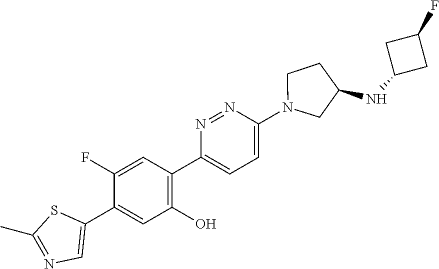 [SMILES: Cc1ncc(-c2cc(O)c(-c3ccc(N4CC[C@@H](N[C@H]5C[C@H](F)C5)C4)nn3)cc2F)s1] |
| 333 | 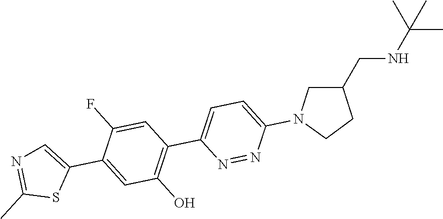 [SMILES: Cc1ncc(-c2cc(O)c(-c3ccc(N4CCC(CNC(C)(C)C)C4)nn3)cc2F)s1] |
| 334 | 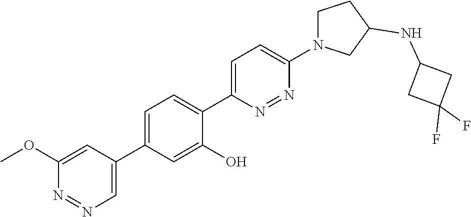 [SMILES: COc1cc(-c2ccc(-c3ccc(N4CCC(NC5CC(F)(F)C5)C4)nn3)c(O)c2)cnn1] |
| 335 | 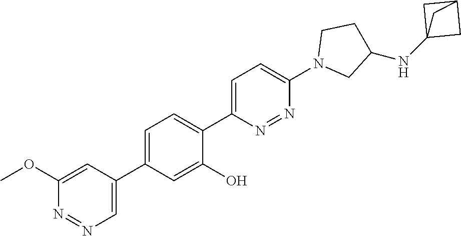 [SMILES: COc1cc(-c2ccc(-c3ccc(N4CCC(NC56CC(C5)C6)C4)nn3)c(O)c2)cnn1] |
| 336 | 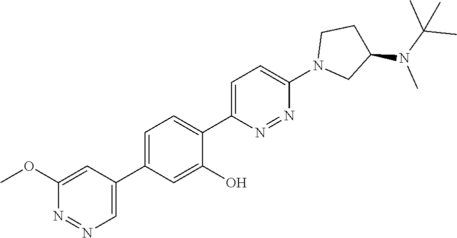 [SMILES: COc1cc(-c2ccc(-c3ccc(N4CC[C@@H](N(C)C(C)(C)C)C4)nn3)c(O)c2)cnn1] |
| 337 | 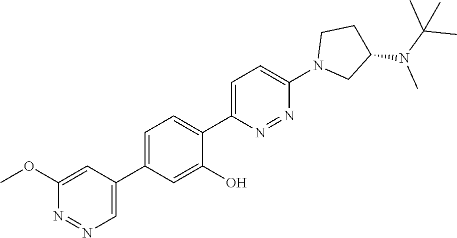 [SMILES: COc1cc(-c2ccc(-c3ccc(N4CC[C@H](N(C)C(C)(C)C)C4)nn3)c(O)c2)cnn1] |
| 338 | 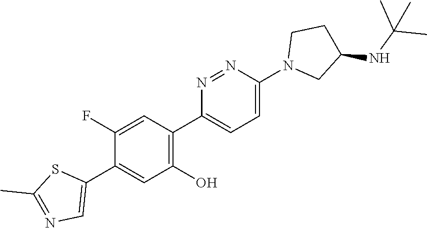 [SMILES: Cc1ncc(-c2cc(O)c(-c3ccc(N4CC[C@@H](NC(C)(C)C)C4)nn3)cc2F)s1] |
| 339 | 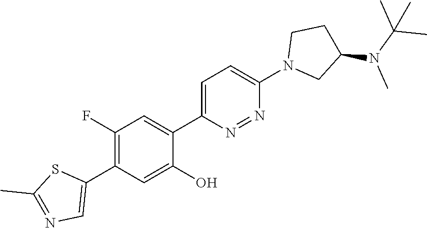 [SMILES: Cc1ncc(-c2cc(O)c(-c3ccc(N4CC[C@@H](N(C)C(C)(C)C)C4)nn3)cc2F)s1] |
| 340 | 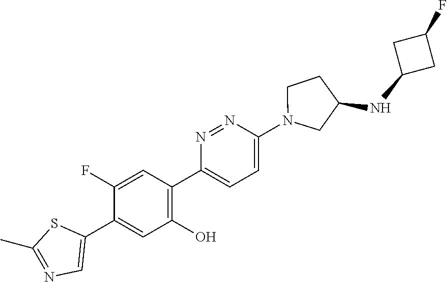 [SMILES: Cc1ncc(-c2cc(O)c(-c3ccc(N4CC[C@@H](N[C@H]5C[C@@H](F)C5)C4)nn3)cc2F)s1] |
| 341 | 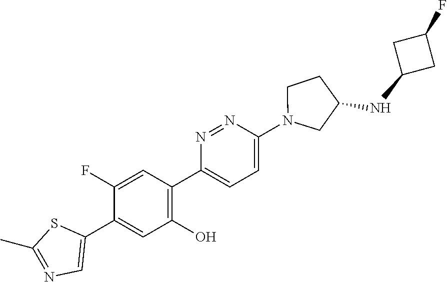 [SMILES: Cc1ncc(-c2cc(O)c(-c3ccc(N4CC[C@H](N[C@H]5C[C@@H](F)C5)C4)nn3)cc2F)s1] |
| 342 | 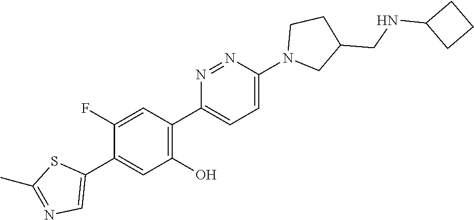 [SMILES: Cc1ncc(-c2cc(O)c(-c3ccc(N4CCC(CNC5CCC5)C4)nn3)cc2F)s1] |
| 343 | 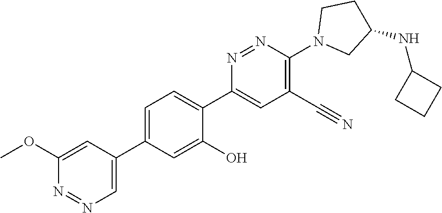 [SMILES: COc1cc(-c2ccc(-c3cc(C#N)c(N4CC[C@H](NC5CCC5)C4)nn3)c(O)c2)cnn1] |
| 344 | 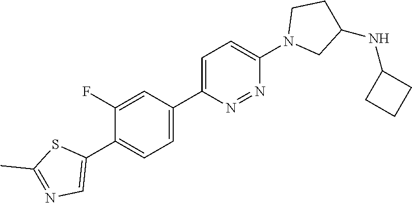 [SMILES: Cc1ncc(-c2ccc(-c3ccc(N4CCC(NC5CCC5)C4)nn3)cc2F)s1] |
| 345 | 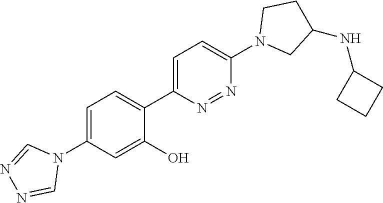 [SMILES: Oc1cc(-n2cnnc2)ccc1-c1ccc(N2CCC(NC3CCC3)C2)nn1] |
| 346 | 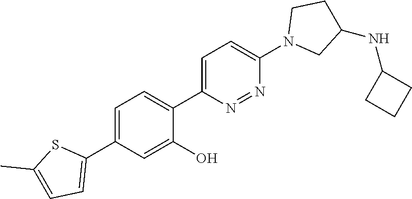 [SMILES: Cc1ccc(-c2ccc(-c3ccc(N4CCC(NC5CCC5)C4)nn3)c(O)c2)s1] |
| 347 | 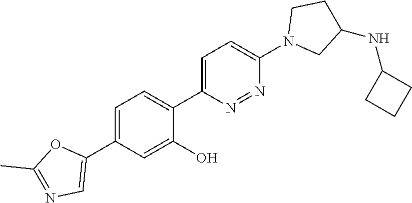 [SMILES: Cc1ncc(-c2ccc(-c3ccc(N4CCC(NC5CCC5)C4)nn3)c(O)c2)o1] |
| 348 | 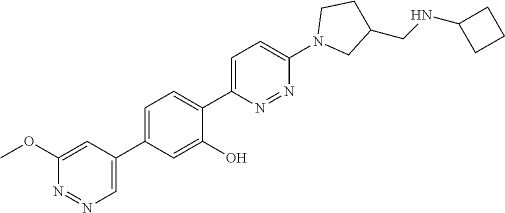 [SMILES: COc1cc(-c2ccc(-c3ccc(N4CCC(CNC5CCC5)C4)nn3)c(O)c2)cnn1] |
| 349 | 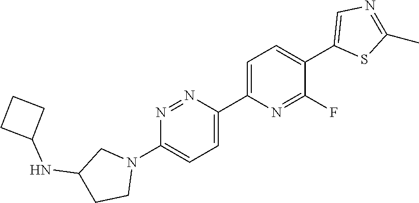 [SMILES: Cc1ncc(-c2ccc(-c3ccc(N4CCC(NC5CCC5)C4)nn3)nc2F)s1] |
| 350 | 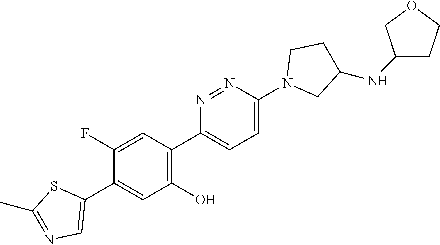 [SMILES: Cc1ncc(-c2cc(O)c(-c3ccc(N4CCC(NC5CCOC5)C4)nn3)cc2F)s1] |
| 351 | 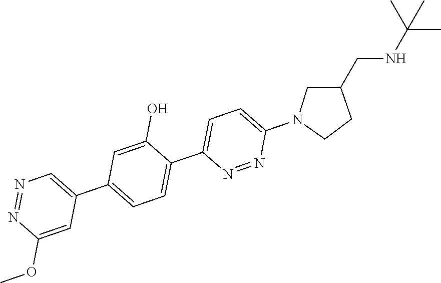 [SMILES: COc1cc(-c2ccc(-c3ccc(N4CCC(CNC(C)(C)C)C4)nn3)c(O)c2)cnn1] |
| 352 | 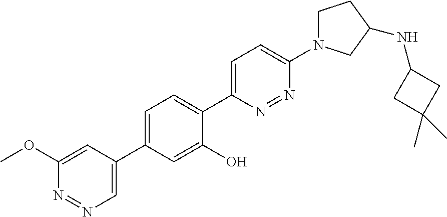 |
| 353 | 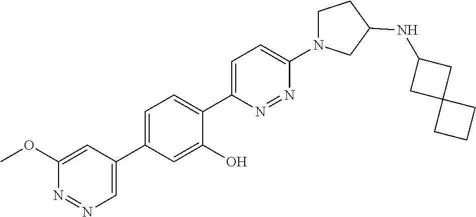 [SMILES: COc1cc(-c2ccc(-c3ccc(N4CCC(NC5CC6(CCC6)C5)C4)nn3)c(O)c2)cnn1] |
| 354 | 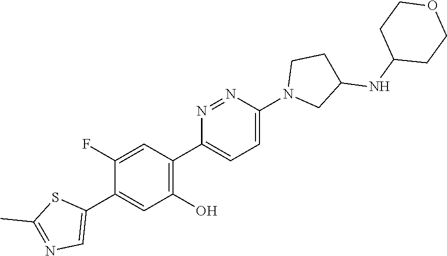 [SMILES: Cc1ncc(-c2cc(O)c(-c3ccc(N4CCC(NC5CCOCC5)C4)nn3)cc2F)s1] |
| 355 | 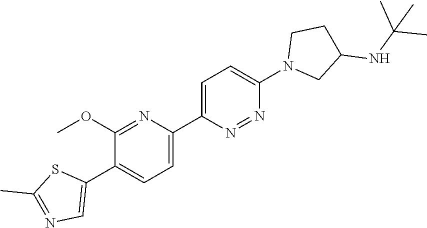 [SMILES: COc1nc(-c2ccc(N3CCC(NC(C)(C)C)C3)nn2)ccc1-c1cnc(C)s1] |
| 356 | 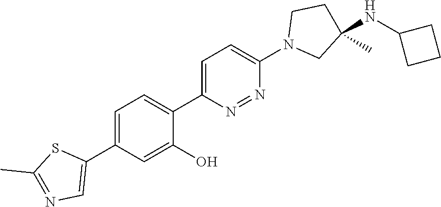 [SMILES: Cc1ncc(-c2ccc(-c3ccc(N4CC[C@@](C)(NC5CCC5)C4)nn3)c(O)c2)s1] |
| 357 | 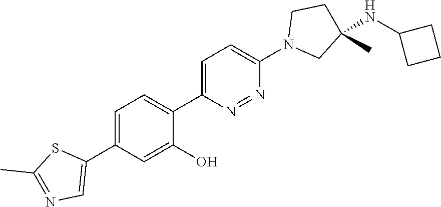 [SMILES: Cc1ncc(-c2ccc(-c3ccc(N4CC[C@](C)(NC5CCC5)C4)nn3)c(O)c2)s1] |
| 358 | 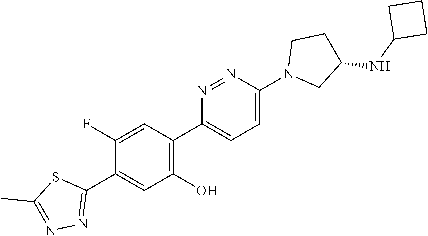 [SMILES: Cc1nnc(-c2cc(O)c(-c3ccc(N4CC[C@H](NC5CCC5)C4)nn3)cc2F)s1] |
| 359 | 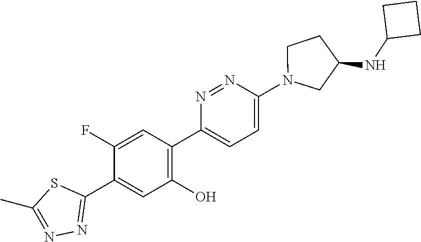 [SMILES: Cc1nnc(-c2cc(O)c(-c3ccc(N4CC[C@@H](NC5CCC5)C4)nn3)cc2F)s1] |
| 360 | 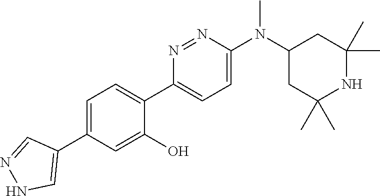 [SMILES: CN(c1ccc(-c2ccc(-c3cn[nH]c3)cc2O)nn1)C1CC(C)(C)NC(C)(C)C1] |
| 361 | 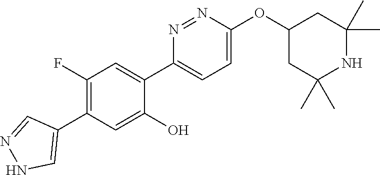 [SMILES: CC1(C)CC(Oc2ccc(-c3cc(F)c(-c4cn[nH]c4)cc3O)nn2)CC(C)(C)N1] |
| 362 | 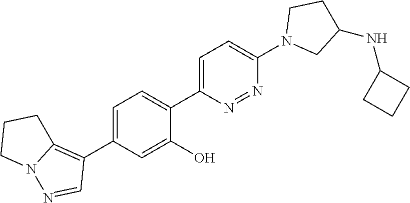 [SMILES: Oc1cc(-c2cnn3c2CCC3)ccc1-c1ccc(N2CCC(NC3CCC3)C2)nn1] |
| 363 | 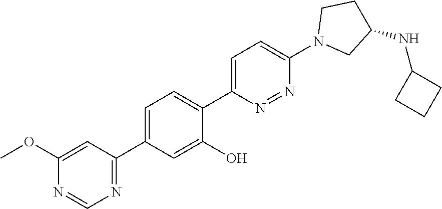 [SMILES: COc1cc(-c2ccc(-c3ccc(N4CC[C@H](NC5CCC5)C4)nn3)c(O)c2)ncn1] |
| 364 | 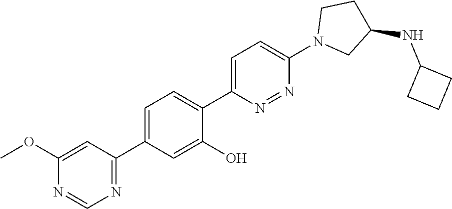 [SMILES: COc1cc(-c2ccc(-c3ccc(N4CC[C@@H](NC5CCC5)C4)nn3)c(O)c2)ncn1] |
| 365 | 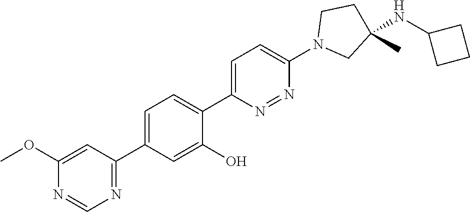 [SMILES: COc1cc(-c2ccc(-c3ccc(N4CC[C@](C)(NC5CCC5)C4)nn3)c(O)c2)ncn1] |
| 366 | 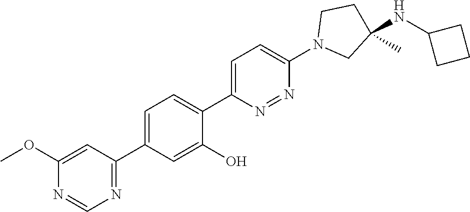 [SMILES: COc1cc(-c2ccc(-c3ccc(N4CC[C@@](C)(NC5CCC5)C4)nn3)c(O)c2)ncn1] |
| 367 | 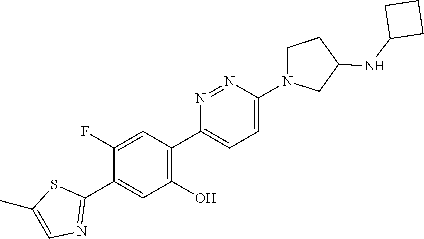 [SMILES: Cc1cnc(-c2cc(O)c(-c3ccc(N4CCC(NC5CCC5)C4)nn3)cc2F)s1] |
| 368 | 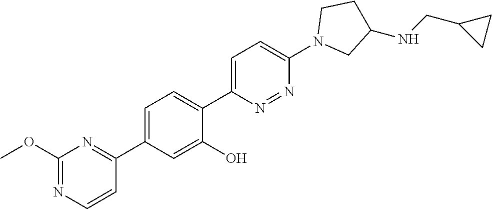 [SMILES: COc1nccc(-c2ccc(-c3ccc(N4CCC(NCC5CC5)C4)nn3)c(O)c2)n1] |
| 369 | 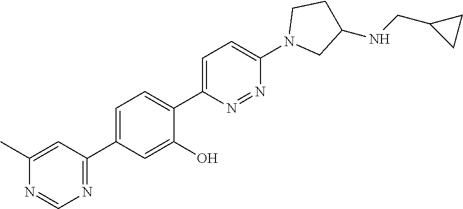 [SMILES: Cc1cc(-c2ccc(-c3ccc(N4CCC(NCC5CC5)C4)nn3)c(O)c2)ncn1] |
| 370 | 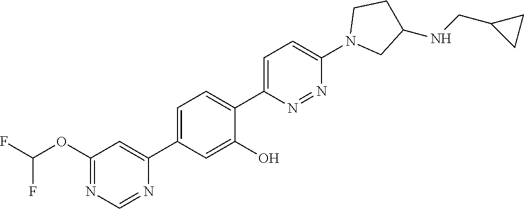 [SMILES: Oc1cc(-c2cc(OC(F)F)ncn2)ccc1-c1ccc(N2CCC(NCC3CC3)C2)nn1] |
| 371 | 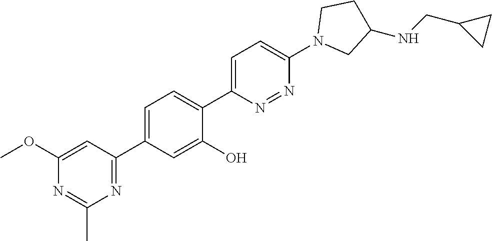 [SMILES: COc1cc(-c2ccc(-c3ccc(N4CCC(NCC5CC5)C4)nn3)c(O)c2)nc(C)n1] |
| 372 | 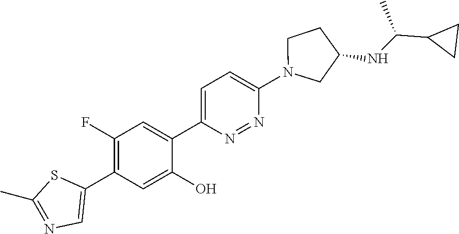 [SMILES: Cc1ncc(-c2cc(O)c(-c3ccc(N4CC[C@H](N[C@H](C)C5CC5)C4)nn3)cc2F)s1] |
| 373 | 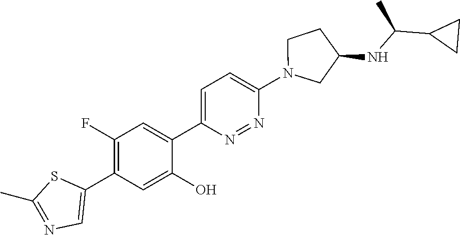 [SMILES: Cc1ncc(-c2cc(O)c(-c3ccc(N4CC[C@@H](N[C@@H](C)C5CC5)C4)nn3)cc2F)s1] |
| 374 | 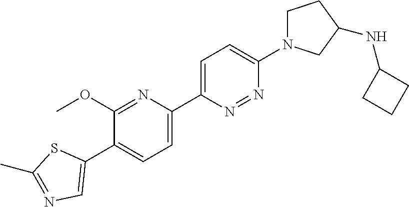 [SMILES: COc1nc(-c2ccc(N3CCC(NC4CCC4)C3)nn2)ccc1-c1cnc(C)s1] |
| 375 | 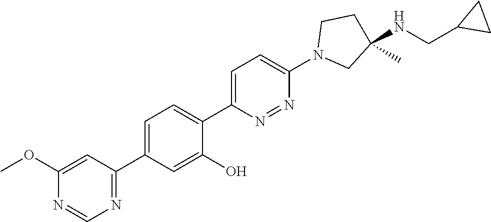 [SMILES: COc1cc(-c2ccc(-c3ccc(N4CC[C@@](C)(NCC5CC5)C4)nn3)c(O)c2)ncn1] |
| 376 | 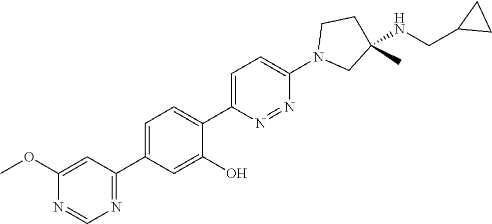 [SMILES: COc1cc(-c2ccc(-c3ccc(N4CC[C@](C)(NCC5CC5)C4)nn3)c(O)c2)ncn1] |
| 377 | 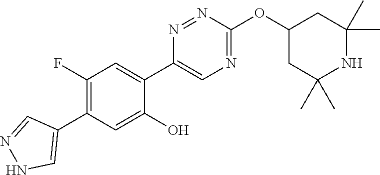 [SMILES: CC1(C)CC(Oc2ncc(-c3cc(F)c(-c4cn[nH]c4)cc3O)nn2)CC(C)(C)N1] |
| 378 | 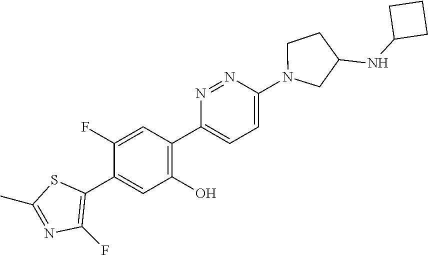 [SMILES: Cc1nc(F)c(-c2cc(O)c(-c3ccc(N4CCC(NC5CCC5)C4)nn3)cc2F)s1] |
| 379 | 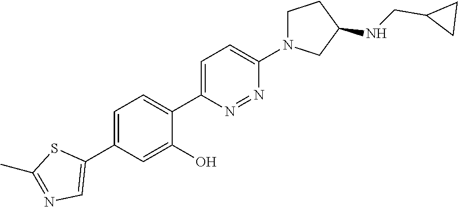 [SMILES: Cc1ncc(-c2ccc(-c3ccc(N4CC[C@@H](NCC5CC5)C4)nn3)c(O)c2)s1] |
| 380 | 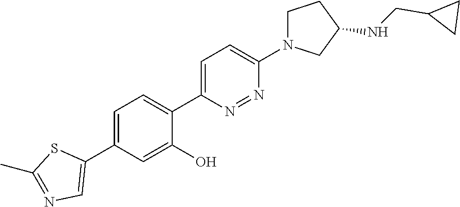 [SMILES: Cc1ncc(-c2ccc(-c3ccc(N4CC[C@H](NCC5CC5)C4)nn3)c(O)c2)s1] |
| 381 | 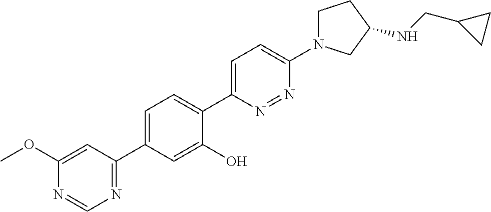 [SMILES: COc1cc(-c2ccc(-c3ccc(N4CC[C@H](NCC5CC5)C4)nn3)c(O)c2)ncn1] |
| 382 | 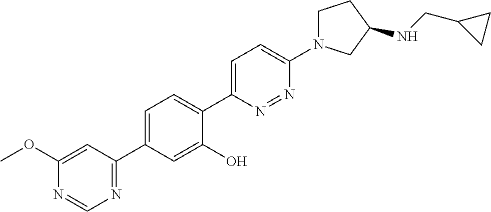 [SMILES: COc1cc(-c2ccc(-c3ccc(N4CC[C@@H](NCC5CC5)C4)nn3)c(O)c2)ncn1] |
| 383 | 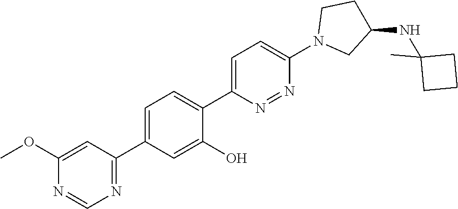 [SMILES: COc1cc(-c2ccc(-c3ccc(N4CC[C@@H](NC5(C)CCC5)C4)nn3)c(O)c2)ncn1] |
| 384 | 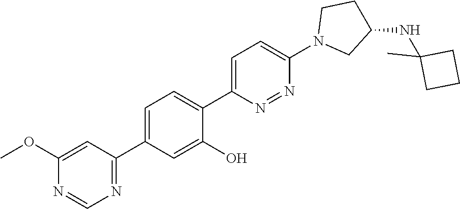 [SMILES: COc1cc(-c2ccc(-c3ccc(N4CC[C@H](NC5(C)CCC5)C4)nn3)c(O)c2)ncn1] |
| 385 | 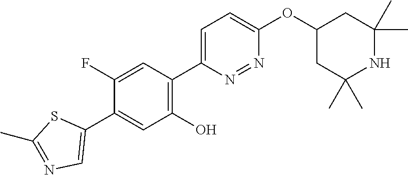 |
| 386 | 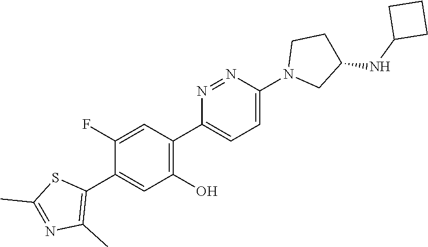 [SMILES: Cc1nc(C)c(-c2cc(O)c(-c3ccc(N4CC[C@H](NC5CCC5)C4)nn3)cc2F)s1] |
| 387 | 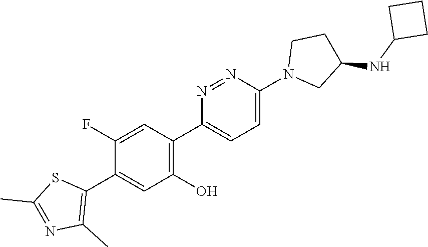 [SMILES: Cc1nc(C)c(-c2cc(O)c(-c3ccc(N4CC[C@@H](NC5CCC5)C4)nn3)cc2F)s1] |
| 388 | 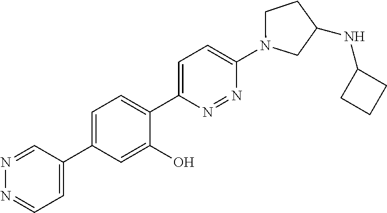 [SMILES: Oc1cc(-c2ccnnc2)ccc1-c1ccc(N2CCC(NC3CCC3)C2)nn1] |
| 389 | 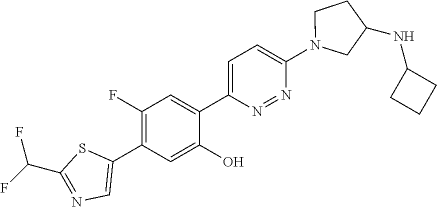 [SMILES: Oc1cc(-c2cnc(C(F)F)s2)c(F)cc1-c1ccc(N2CCC(NC3CCC3)C2)nn1] |
| 390 | 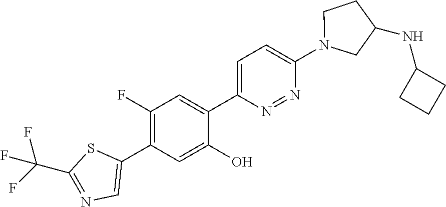 [SMILES: Oc1cc(-c2cnc(C(F)(F)F)s2)c(F)cc1-c1ccc(N2CCC(NC3CCC3)C2)nn1] |
| 391 | 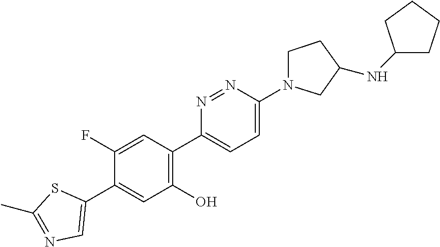 [SMILES: Cc1ncc(-c2cc(O)c(-c3ccc(N4CCC(NC5CCCC5)C4)nn3)cc2F)s1] |
| 392 | 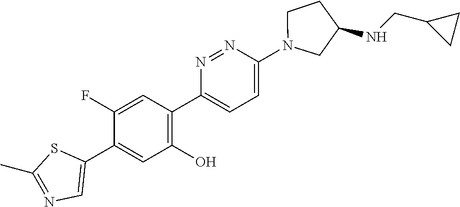 [SMILES: Cc1ncc(-c2cc(O)c(-c3ccc(N4CC[C@@H](NCC5CC5)C4)nn3)cc2F)s1] |
| 393 | 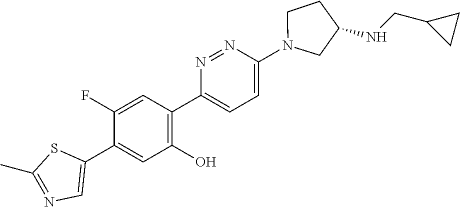 [SMILES: Cc1ncc(-c2cc(O)c(-c3ccc(N4CC[C@H](NCC5CC5)C4)nn3)cc2F)s1] |
| 394 | 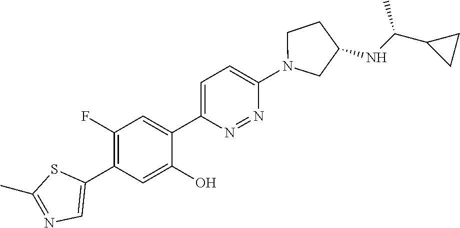 [SMILES: Cc1ncc(-c2cc(O)c(-c3ccc(N4CC[C@H](N[C@H](C)C5CC5)C4)nn3)cc2F)s1] |
| 395 | 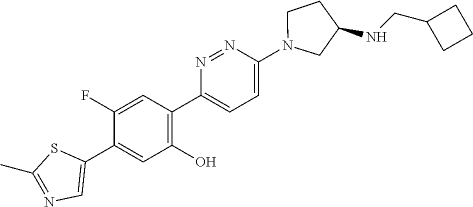 [SMILES: Cc1ncc(-c2cc(O)c(-c3ccc(N4CC[C@@H](NCC5CCC5)C4)nn3)cc2F)s1] |
| 396 | 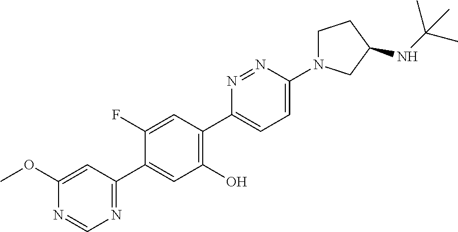 [SMILES: COc1cc(-c2cc(O)c(-c3ccc(N4CC[C@@H](NC(C)(C)C)C4)nn3)cc2F)ncn1] |
| 397 | 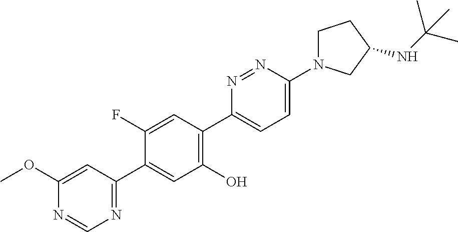 [SMILES: COc1cc(-c2cc(O)c(-c3ccc(N4CC[C@H](NC(C)(C)C)C4)nn3)cc2F)ncn1] |
| 398 | 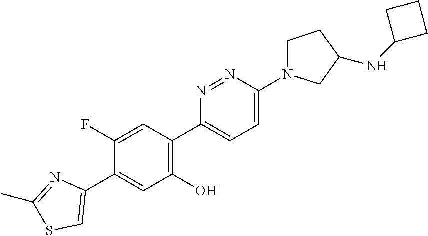 [SMILES: Cc1nc(-c2cc(O)c(-c3ccc(N4CCC(NC5CCC5)C4)nn3)cc2F)cs1] |
| 399 | 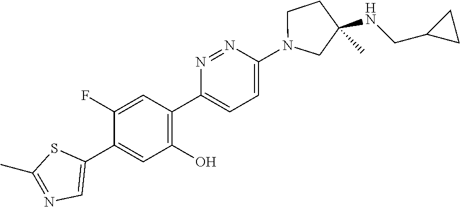 [SMILES: Cc1ncc(-c2cc(O)c(-c3ccc(N4CC[C@@](C)(NCC5CC5)C4)nn3)cc2F)s1] |
| 400 | 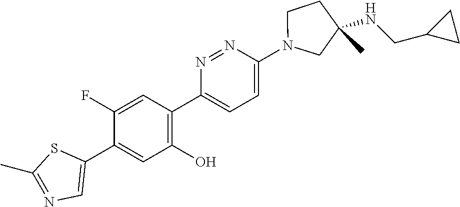 [SMILES: Cc1ncc(-c2cc(O)c(-c3ccc(N4CC[C@](C)(NCC5CC5)C4)nn3)cc2F)s1] |
| 401 | 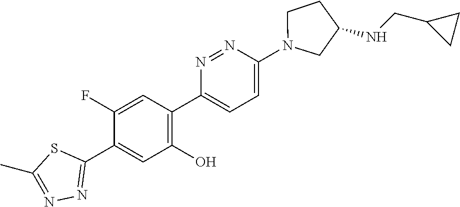 [SMILES: Cc1nnc(-c2cc(O)c(-c3ccc(N4CC[C@H](NCC5CC5)C4)nn3)cc2F)s1] |
| 402 | 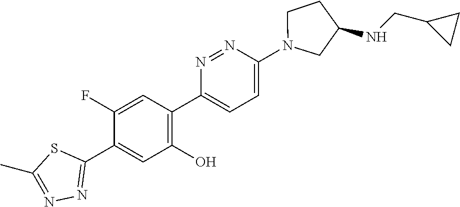 [SMILES: Cc1nnc(-c2cc(O)c(-c3ccc(N4CC[C@@H](NCC5CC5)C4)nn3)cc2F)s1] |
| 403 | 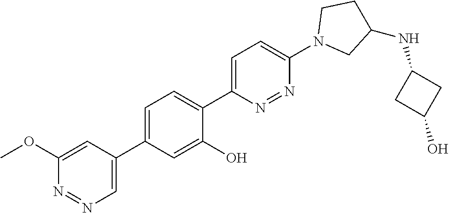 [SMILES: COc1cc(-c2ccc(-c3ccc(N4CCC(N[C@H]5C[C@@H](O)C5)C4)nn3)c(O)c2)cnn1] |
| 404 | 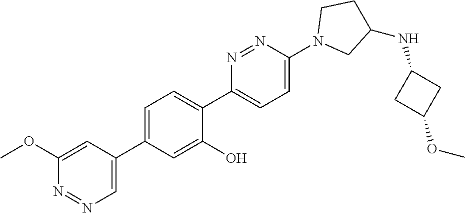 |
| 405 | 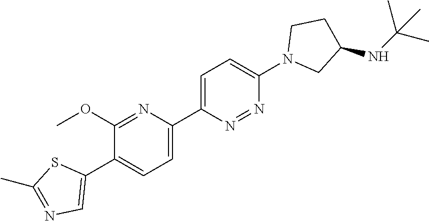 [SMILES: COc1nc(-c2ccc(N3CC[C@@H](NC(C)(C)C)C3)nn2)ccc1-c1cnc(C)s1] |
| 406 | 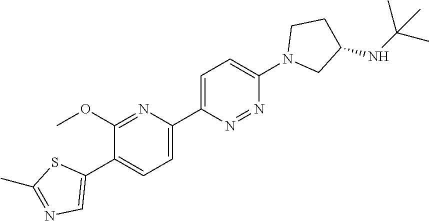 [SMILES: COc1nc(-c2ccc(N3CC[C@H](NC(C)(C)C)C3)nn2)ccc1-c1cnc(C)s1] |
| 407 | 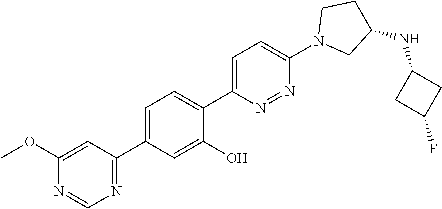 [SMILES: COc1cc(-c2ccc(-c3ccc(N4CC[C@H](N[C@H]5C[C@@H](F)C5)C4)nn3)c(O)c2)ncn1] |
| 408 | 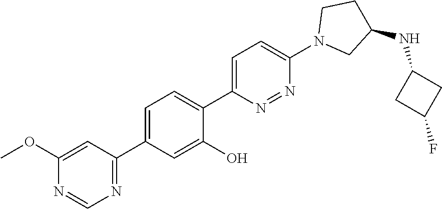 [SMILES: COc1cc(-c2ccc(-c3ccc(N4CC[C@@H](N[C@H]5C[C@@H](F)C5)C4)nn3)c(O)c2)ncn1] |
| 409 | 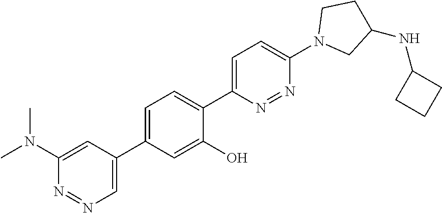 [SMILES: CN(C)c1cc(-c2ccc(-c3ccc(N4CCC(NC5CCC5)C4)nn3)c(O)c2)cnn1] |
| 410 | 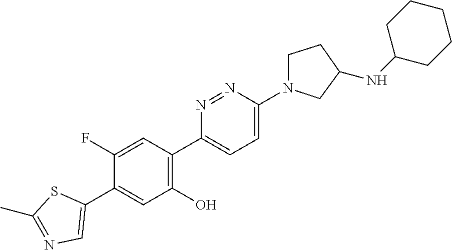 [SMILES: Cc1ncc(-c2cc(O)c(-c3ccc(N4CCC(NC5CCCCC5)C4)nn3)cc2F)s1] |
| 411 | 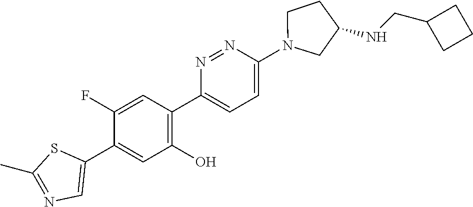 [SMILES: Cc1ncc(-c2cc(O)c(-c3ccc(N4CC[C@H](NCC5CCC5)C4)nn3)cc2F)s1] |
| 412 | 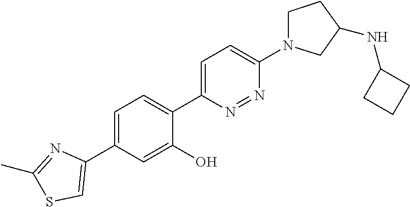 [SMILES: Cc1nc(-c2ccc(-c3ccc(N4CCC(NC5CCC5)C4)nn3)c(O)c2)cs1] |
| 413 | 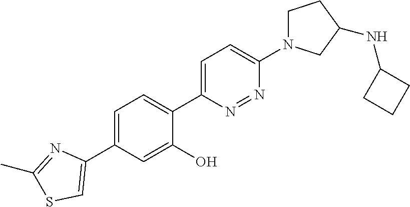 [SMILES: Cc1nc(-c2ccc(-c3ccc(N4CCC(NC5CCC5)C4)nn3)c(O)c2)cs1] |
| 414 | 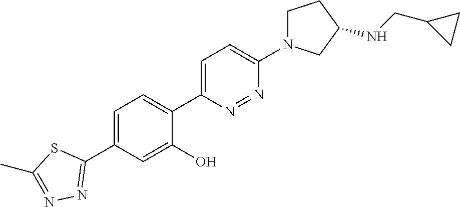 [SMILES: Cc1nnc(-c2ccc(-c3ccc(N4CC[C@H](NCC5CC5)C4)nn3)c(O)c2)s1] |
| 415 | 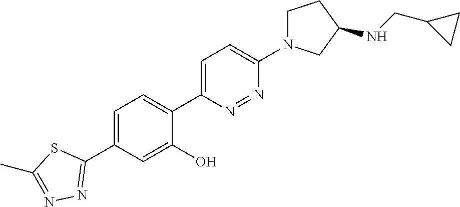 [SMILES: Cc1nnc(-c2ccc(-c3ccc(N4CC[C@@H](NCC5CC5)C4)nn3)c(O)c2)s1] |
| 416 | 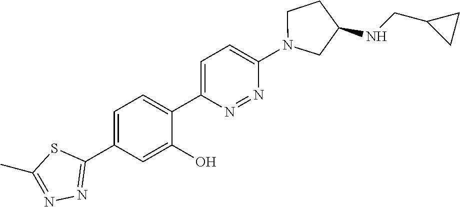 [SMILES: Cc1nnc(-c2ccc(-c3ccc(N4CC[C@@H](NCC5CC5)C4)nn3)c(O)c2)s1] |
| 417 | 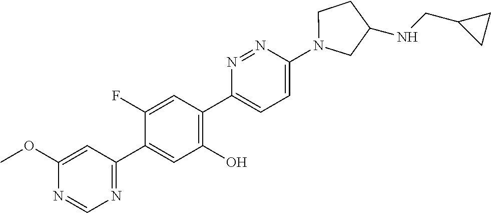 [SMILES: COc1cc(-c2cc(O)c(-c3ccc(N4CCC(NCC5CC5)C4)nn3)cc2F)ncn1] |
| 418 | 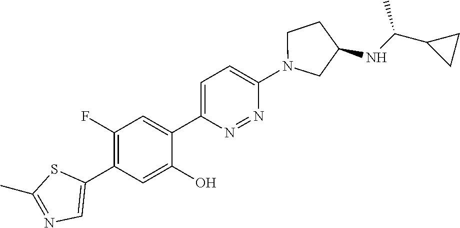 [SMILES: Cc1ncc(-c2cc(O)c(-c3ccc(N4CC[C@@H](N[C@H](C)C5CC5)C4)nn3)cc2F)s1] |
| 419 | 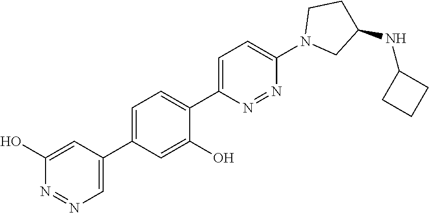 [SMILES: Oc1cc(-c2ccc(-c3ccc(N4CC[C@@H](NC5CCC5)C4)nn3)c(O)c2)cnn1] |
| 420 | 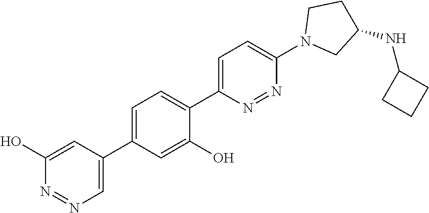 [SMILES: Oc1cc(-c2ccc(-c3ccc(N4CC[C@H](NC5CCC5)C4)nn3)c(O)c2)cnn1] |
| 421 | 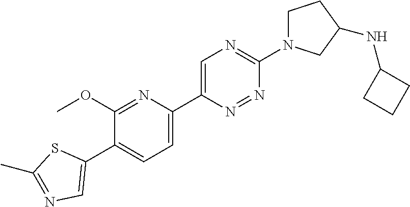 [SMILES: COc1nc(-c2cnc(N3CCC(NC4CCC4)C3)nn2)ccc1-c1cnc(C)s1] |
| 422 | 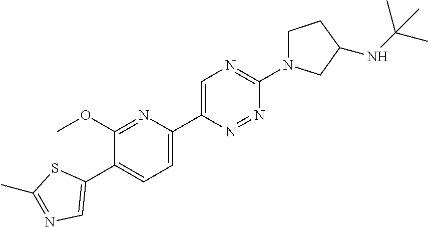 [SMILES: COc1nc(-c2cnc(N3CCC(NC(C)(C)C)C3)nn2)ccc1-c1cnc(C)s1] |
| 423 | 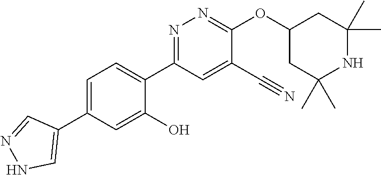 [SMILES: CC1(C)CC(Oc2nnc(-c3ccc(-c4cn[nH]c4)cc3O)cc2C#N)CC(C)(C)N1] |
| 424 | 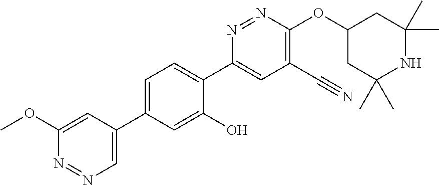 |
| 425 | 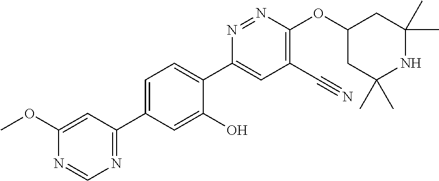 |
| 426 | 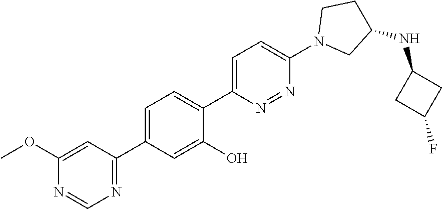 [SMILES: COc1cc(-c2ccc(-c3ccc(N4CC[C@H](N[C@H]5C[C@H](F)C5)C4)nn3)c(O)c2)ncn1] |
| 427 | 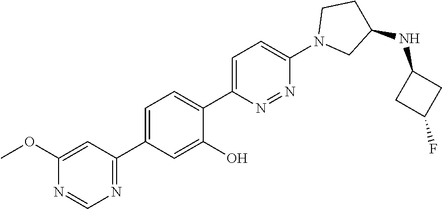 [SMILES: COc1cc(-c2ccc(-c3ccc(N4CC[C@@H](N[C@H]5C[C@H](F)C5)C4)nn3)c(O)c2)ncn1] |
| 428 | 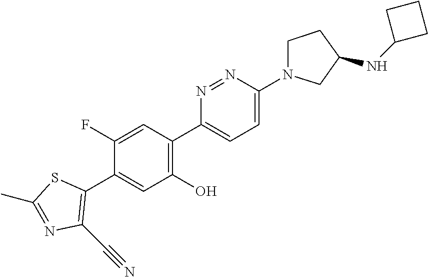 [SMILES: Cc1nc(C#N)c(-c2cc(O)c(-c3ccc(N4CC[C@@H](NC5CCC5)C4)nn3)cc2F)s1] |
| 429 | 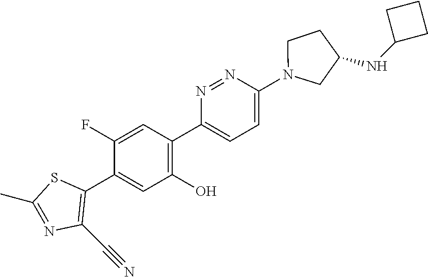 [SMILES: Cc1nc(C#N)c(-c2cc(O)c(-c3ccc(N4CC[C@H](NC5CCC5)C4)nn3)cc2F)s1] |
| 430 | 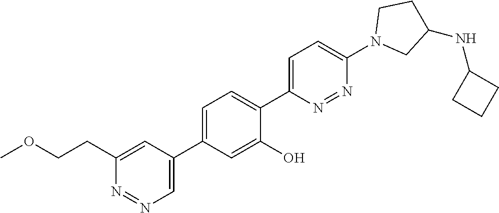 [SMILES: COCCc1cc(-c2ccc(-c3ccc(N4CCC(NC5CCC5)C4)nn3)c(O)c2)cnn1] |
| 431 | 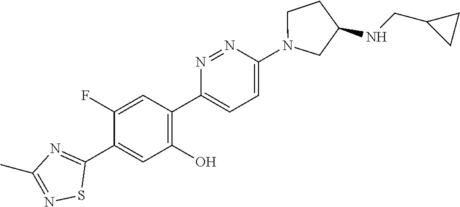 [SMILES: Cc1nsc(-c2cc(O)c(-c3ccc(N4CC[C@@H](NCC5CC5)C4)nn3)cc2F)n1] |
| 432 | 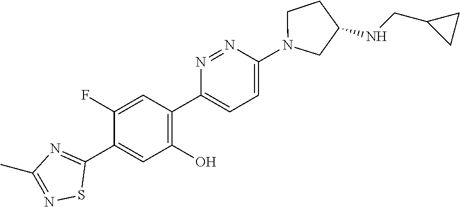 [SMILES: Cc1nsc(-c2cc(O)c(-c3ccc(N4CC[C@H](NCC5CC5)C4)nn3)cc2F)n1] |
| 433 | 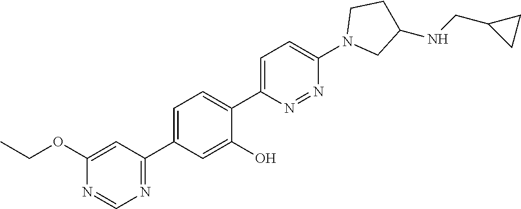 [SMILES: CCOc1cc(-c2ccc(-c3ccc(N4CCC(NCC5CC5)C4)nn3)c(O)c2)ncn1] |
| 434 | 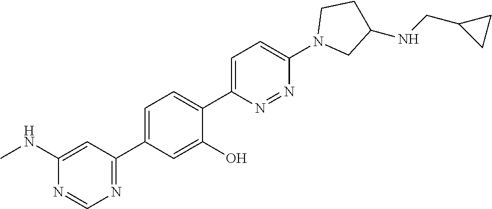 [SMILES: CNc1cc(-c2ccc(-c3ccc(N4CCC(NCC5CC5)C4)nn3)c(O)c2)ncn1] |
| 435 | 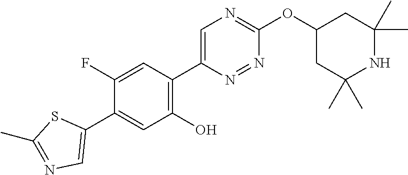 |
| 436 | 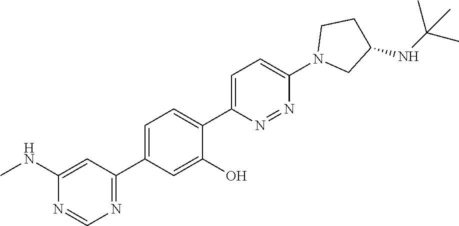 [SMILES: CNc1cc(-c2ccc(-c3ccc(N4CC[C@H](NC(C)(C)C)C4)nn3)c(O)c2)ncn1] |
| 437 | 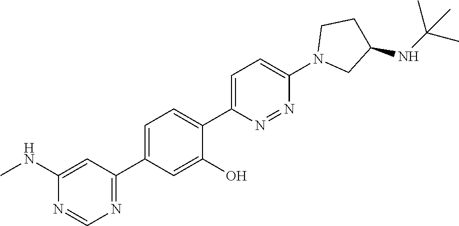 [SMILES: CNc1cc(-c2ccc(-c3ccc(N4CC[C@@H](NC(C)(C)C)C4)nn3)c(O)c2)ncn1] |
| 438 | 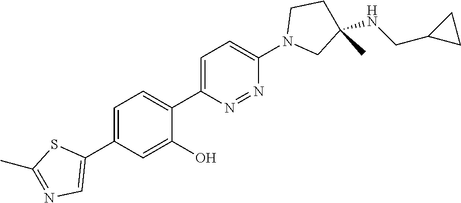 [SMILES: Cc1ncc(-c2ccc(-c3ccc(N4CC[C@](C)(NCC5CC5)C4)nn3)c(O)c2)s1] |
| 439 | 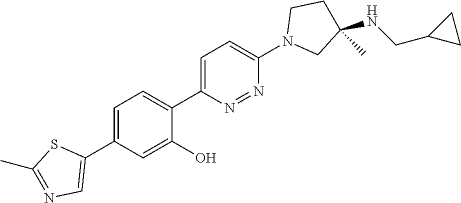 [SMILES: Cc1ncc(-c2ccc(-c3ccc(N4CC[C@@](C)(NCC5CC5)C4)nn3)c(O)c2)s1] |
| 440 | 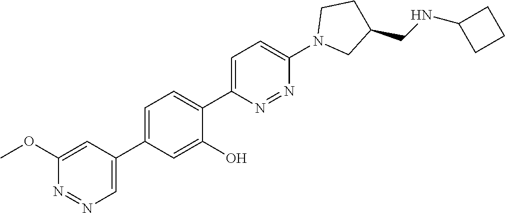 [SMILES: COc1cc(-c2ccc(-c3ccc(N4CC[C@@H](CNC5CCC5)C4)nn3)c(O)c2)cnn1] |
| 441 | 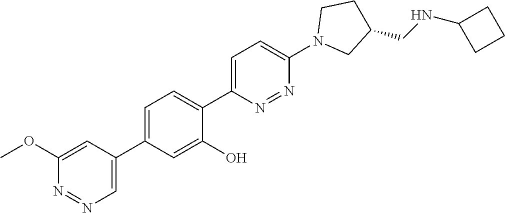 [SMILES: COc1cc(-c2ccc(-c3ccc(N4CC[C@H](CNC5CCC5)C4)nn3)c(O)c2)cnn1] |
| 442 | 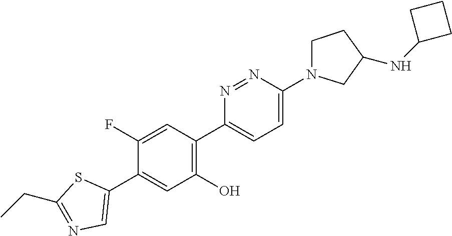 [SMILES: CCc1ncc(-c2cc(O)c(-c3ccc(N4CCC(NC5CCC5)C4)nn3)cc2F)s1] |
| 443 | 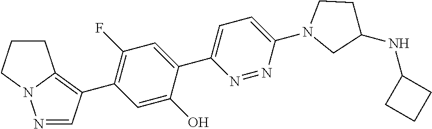 |
| 444 | 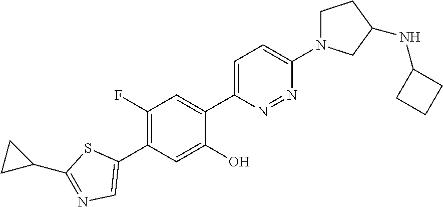 [SMILES: Oc1cc(-c2cnc(C3CC3)s2)c(F)cc1-c1ccc(N2CCC(NC3CCC3)C2)nn1] |
| 445 | 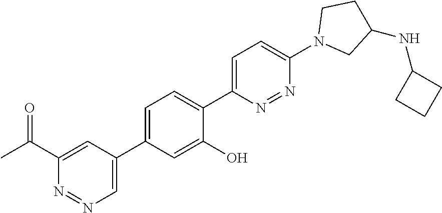 [SMILES: CC(=O)c1cc(-c2ccc(-c3ccc(N4CCC(NC5CCC5)C4)nn3)c(O)c2)cnn1] |
| 446 | 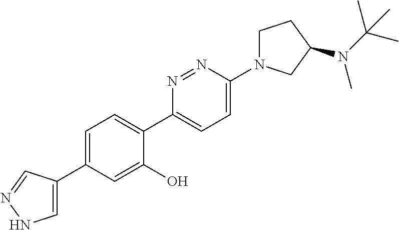 [SMILES: CN([C@@H]1CCN(c2ccc(-c3ccc(-c4cn[nH]c4)cc3O)nn2)C1)C(C)(C)C] |
| 447 | 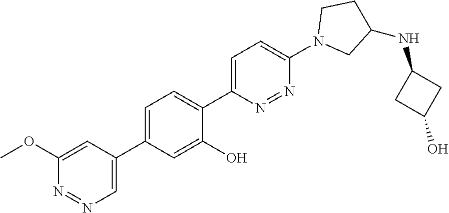 [SMILES: COc1cc(-c2ccc(-c3ccc(N4CCC(N[C@H]5C[C@H](O)C5)C4)nn3)c(O)c2)cnn1] |
| 448 | 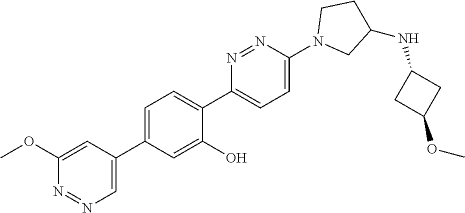 |
| 449 | 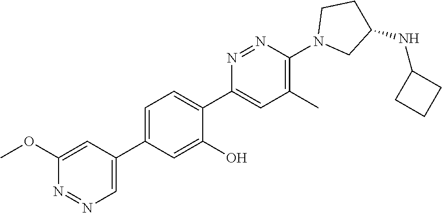 [SMILES: COc1cc(-c2ccc(-c3cc(C)c(N4CC[C@H](NC5CCC5)C4)nn3)c(O)c2)cnn1] |
| 450 | 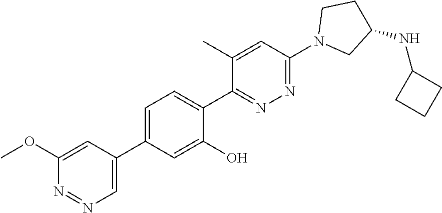 [SMILES: COc1cc(-c2ccc(-c3nnc(N4CC[C@H](NC5CCC5)C4)cc3C)c(O)c2)cnn1] |
| 451 | 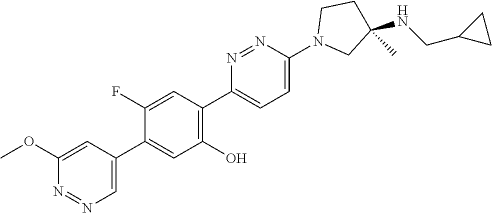 [SMILES: COc1cc(-c2cc(O)c(-c3ccc(N4CC[C@@](C)(NCC5CC5)C4)nn3)cc2F)cnn1] |
| 452 | 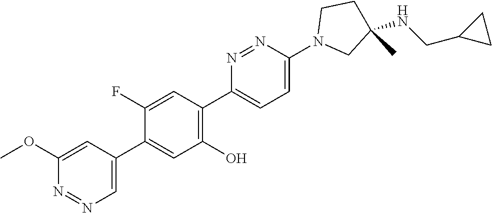 [SMILES: COc1cc(-c2cc(O)c(-c3ccc(N4CC[C@](C)(NCC5CC5)C4)nn3)cc2F)cnn1] |
| 453 | 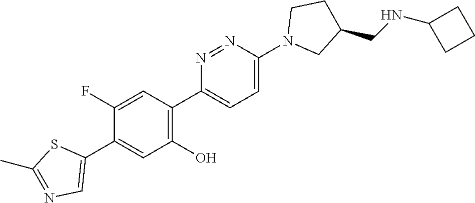 [SMILES: Cc1ncc(-c2cc(O)c(-c3ccc(N4CC[C@@H](CNC5CCC5)C4)nn3)cc2F)s1] |
| 454 | 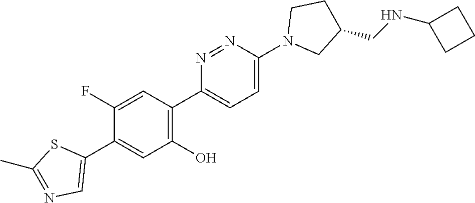 [SMILES: Cc1ncc(-c2cc(O)c(-c3ccc(N4CC[C@H](CNC5CCC5)C4)nn3)cc2F)s1] |
| 455 | 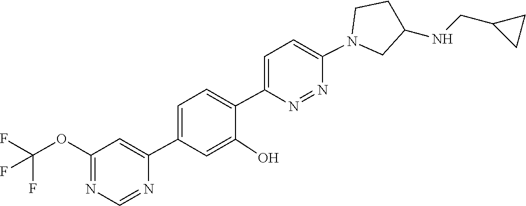 [SMILES: Oc1cc(-c2cc(OC(F)(F)F)ncn2)ccc1-c1ccc(N2CCC(NCC3CC3)C2)nn1] |
| 456 | 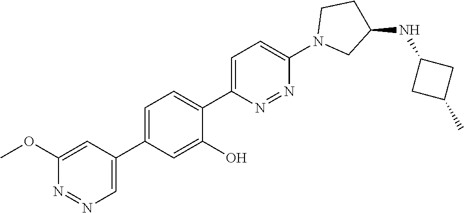 |
| 457 | 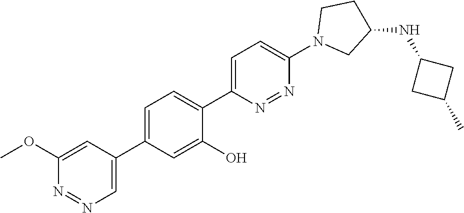 |
| 458 | 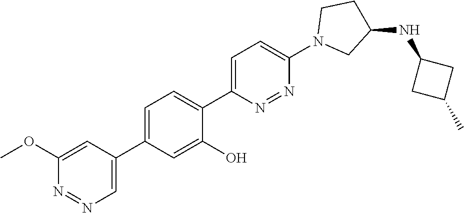 |
| 459 | 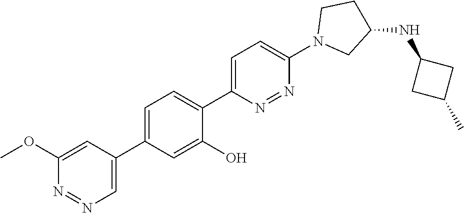 |
| 460 | 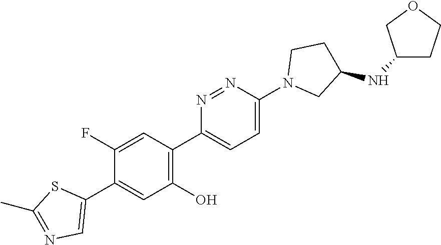 [SMILES: Cc1ncc(-c2cc(O)c(-c3ccc(N4CC[C@@H](N[C@H]5CCOC5)C4)nn3)cc2F)s1] |
| 461 | 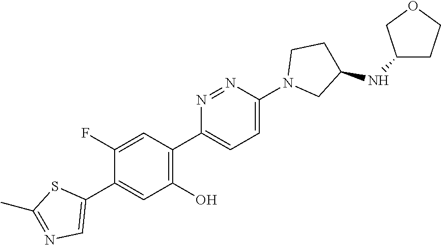 [SMILES: Cc1ncc(-c2cc(O)c(-c3ccc(N4CC[C@@H](N[C@H]5CCOC5)C4)nn3)cc2F)s1] |
| 462 | 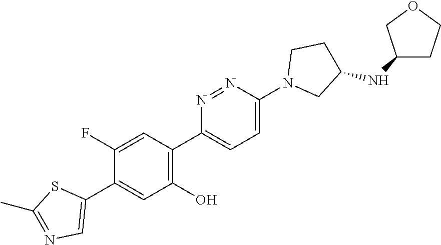 [SMILES: Cc1ncc(-c2cc(O)c(-c3ccc(N4CC[C@H](N[C@@H]5CCOC5)C4)nn3)cc2F)s1] |
| 463 | 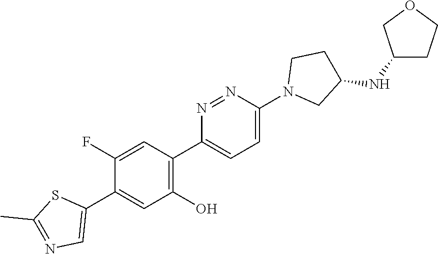 [SMILES: Cc1ncc(-c2cc(O)c(-c3ccc(N4CC[C@H](N[C@H]5CCOC5)C4)nn3)cc2F)s1] |
| 464 | 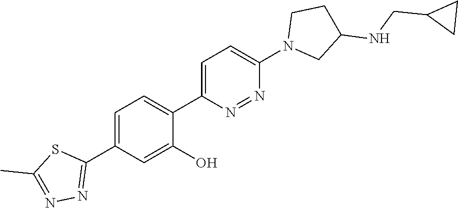 [SMILES: Cc1nnc(-c2ccc(-c3ccc(N4CCC(NCC5CC5)C4)nn3)c(O)c2)s1] |
| 465 | 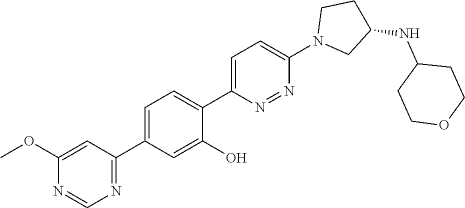 [SMILES: COc1cc(-c2ccc(-c3ccc(N4CC[C@H](NC5CCOCC5)C4)nn3)c(O)c2)ncn1] |
| 466 | 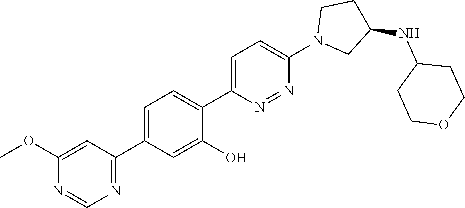 [SMILES: COc1cc(-c2ccc(-c3ccc(N4CC[C@@H](NC5CCOCC5)C4)nn3)c(O)c2)ncn1] |
| 467 | 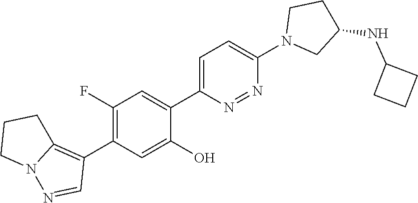 [SMILES: Oc1cc(-c2cnn3c2CCC3)c(F)cc1-c1ccc(N2CC[C@H](NC3CCC3)C2)nn1] |
| 468 | 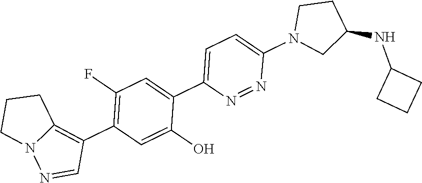 [SMILES: Oc1cc(-c2cnn3c2CCC3)c(F)cc1-c1ccc(N2CC[C@@H](NC3CCC3)C2)nn1] |
| 469 | 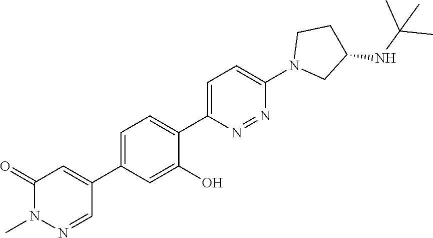 [SMILES: Cn1ncc(-c2ccc(-c3ccc(N4CC[C@H](NC(C)(C)C)C4)nn3)c(O)c2)cc1=O] |
| 470 | 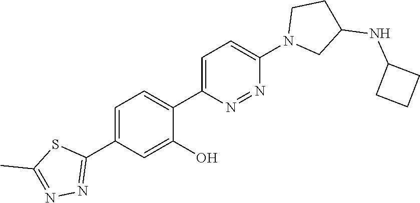 [SMILES: Cc1nnc(-c2ccc(-c3ccc(N4CCC(NC5CCC5)C4)nn3)c(O)c2)s1] |
| 471 | 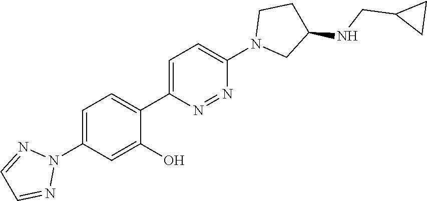 [SMILES: Oc1cc(-n2nccn2)ccc1-c1ccc(N2CC[C@@H](NCC3CC3)C2)nn1] |
| 472 | 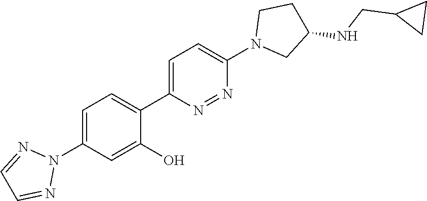 [SMILES: Oc1cc(-n2nccn2)ccc1-c1ccc(N2CC[C@H](NCC3CC3)C2)nn1] |
| 473 | 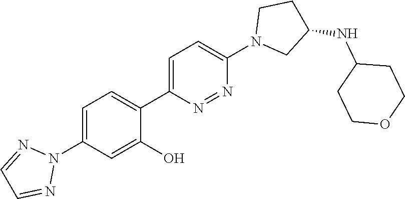 [SMILES: Oc1cc(-n2nccn2)ccc1-c1ccc(N2CC[C@H](NC3CCOCC3)C2)nn1] |
| 474 | 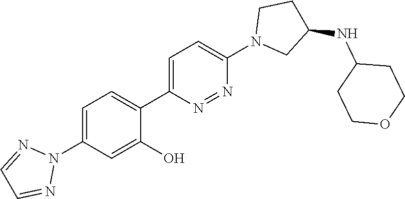 [SMILES: Oc1cc(-n2nccn2)ccc1-c1ccc(N2CC[C@@H](NC3CCOCC3)C2)nn1] |
| 475 | 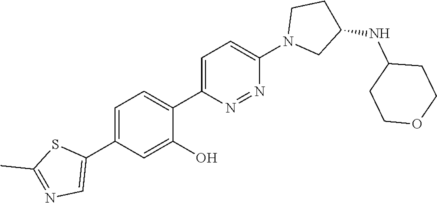 [SMILES: Cc1ncc(-c2ccc(-c3ccc(N4CC[C@H](NC5CCOCC5)C4)nn3)c(O)c2)s1] |
| 476 | 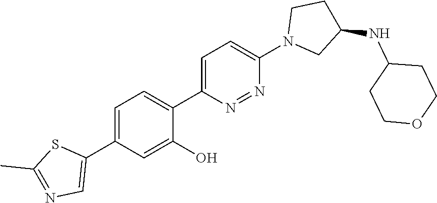 [SMILES: Cc1ncc(-c2ccc(-c3ccc(N4CC[C@@H](NC5CCOCC5)C4)nn3)c(O)c2)s1] |
| 477 | 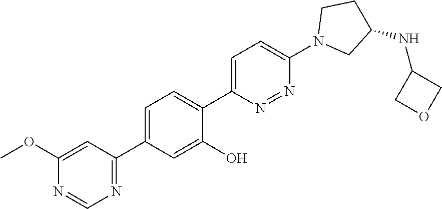 [SMILES: COc1cc(-c2ccc(-c3ccc(N4CC[C@H](NC5COC5)C4)nn3)c(O)c2)ncn1] |
| 478 | 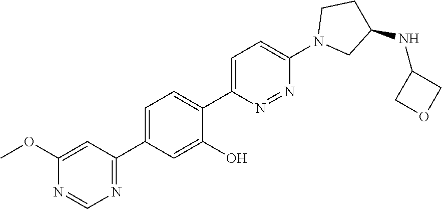 [SMILES: COc1cc(-c2ccc(-c3ccc(N4CC[C@@H](NC5COC5)C4)nn3)c(O)c2)ncn1] |
| 479 | 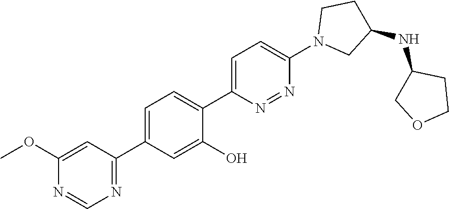 [SMILES: COc1cc(-c2ccc(-c3ccc(N4CC[C@@H](N[C@H]5CCOC5)C4)nn3)c(O)c2)ncn1] |
| 480 | 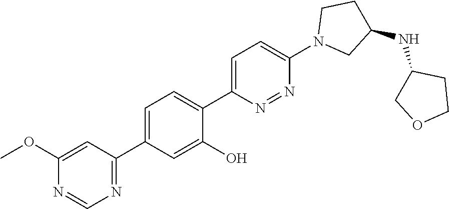 [SMILES: COc1cc(-c2ccc(-c3ccc(N4CC[C@@H](N[C@@H]5CCOC5)C4)nn3)c(O)c2)ncn1] |
| 481 | 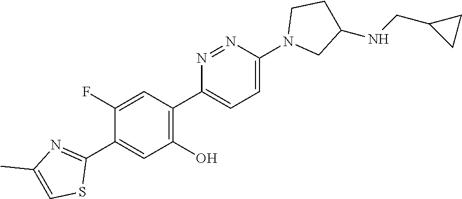 [SMILES: Cc1csc(-c2cc(O)c(-c3ccc(N4CCC(NCC5CC5)C4)nn3)cc2F)n1] |
| 482 | 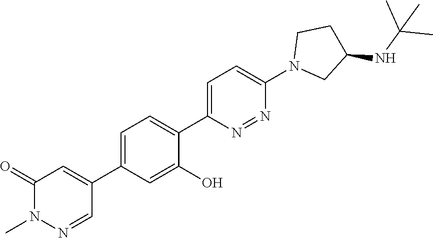 [SMILES: Cn1ncc(-c2ccc(-c3ccc(N4CC[C@@H](NC(C)(C)C)C4)nn3)c(O)c2)cc1=O] |
| 483 | 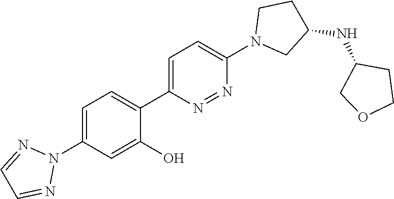 [SMILES: Oc1cc(-n2nccn2)ccc1-c1ccc(N2CC[C@H](N[C@@H]3CCOC3)C2)nn1] |
| 484 | 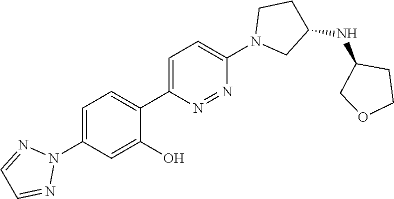 [SMILES: Oc1cc(-n2nccn2)ccc1-c1ccc(N2CC[C@H](N[C@H]3CCOC3)C2)nn1] |
| 485 | 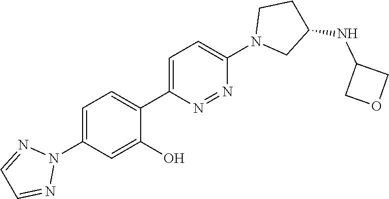 [SMILES: Oc1cc(-n2nccn2)ccc1-c1ccc(N2CC[C@H](NC3COC3)C2)nn1] |
| 486 | 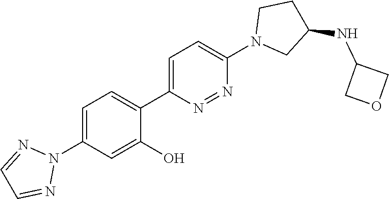 [SMILES: Oc1cc(-n2nccn2)ccc1-c1ccc(N2CC[C@@H](NC3COC3)C2)nn1] |
| 487 | 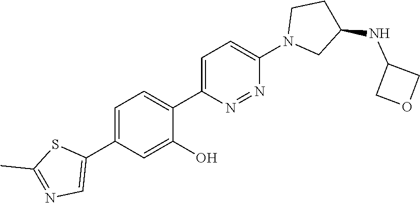 [SMILES: Cc1ncc(-c2ccc(-c3ccc(N4CC[C@@H](NC5COC5)C4)nn3)c(O)c2)s1] |
| 488 | 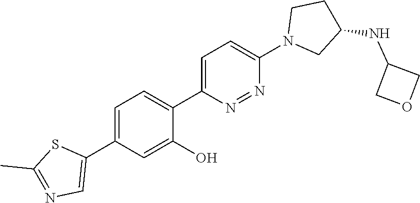 [SMILES: Cc1ncc(-c2ccc(-c3ccc(N4CC[C@H](NC5COC5)C4)nn3)c(O)c2)s1] |
| 489 | 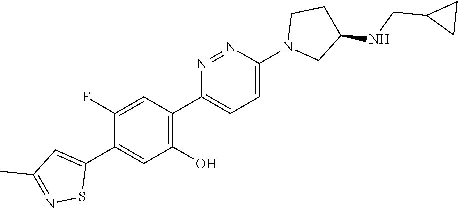 [SMILES: Cc1cc(-c2cc(O)c(-c3ccc(N4CC[C@@H](NCC5CC5)C4)nn3)cc2F)sn1] |
| 490 | 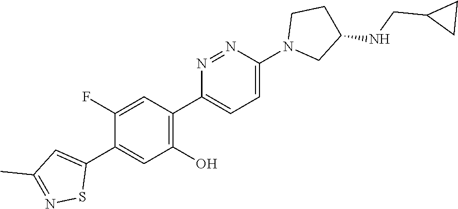 [SMILES: Cc1cc(-c2cc(O)c(-c3ccc(N4CC[C@H](NCC5CC5)C4)nn3)cc2F)sn1] |
| 491 | 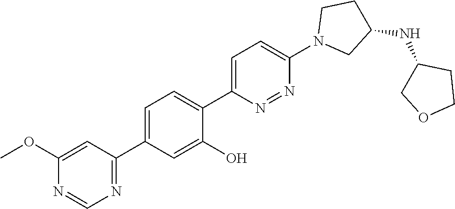 [SMILES: COc1cc(-c2ccc(-c3ccc(N4CC[C@H](N[C@@H]5CCOC5)C4)nn3)c(O)c2)ncn1] |
| 492 | 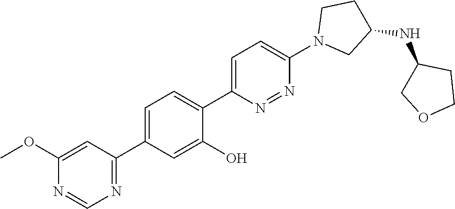 [SMILES: COc1cc(-c2ccc(-c3ccc(N4CC[C@H](N[C@H]5CCOC5)C4)nn3)c(O)c2)ncn1] |
| 493 | 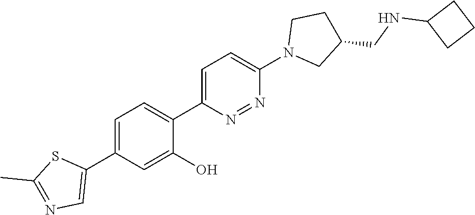 [SMILES: Cc1ncc(-c2ccc(-c3ccc(N4CC[C@H](CNC5CCC5)C4)nn3)c(O)c2)s1] |
| 494 | 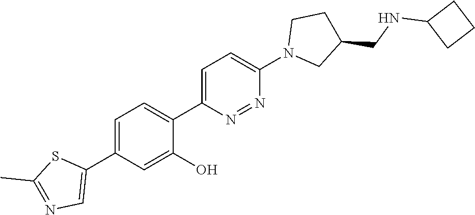 [SMILES: Cc1ncc(-c2ccc(-c3ccc(N4CC[C@@H](CNC5CCC5)C4)nn3)c(O)c2)s1] |
| 495 | 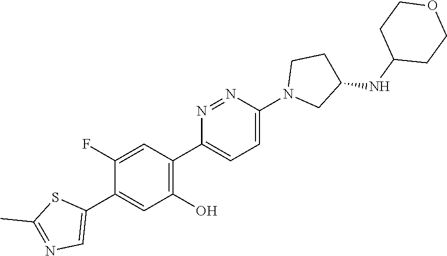 [SMILES: Cc1ncc(-c2cc(O)c(-c3ccc(N4CC[C@H](NC5CCOCC5)C4)nn3)cc2F)s1] |
| 496 | 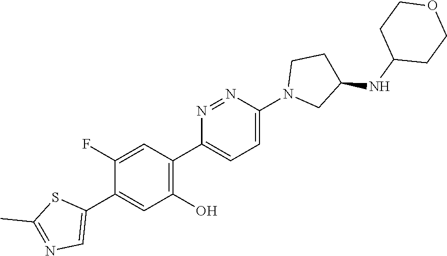 [SMILES: Cc1ncc(-c2cc(O)c(-c3ccc(N4CC[C@@H](NC5CCOCC5)C4)nn3)cc2F)s1] |
| 497 | 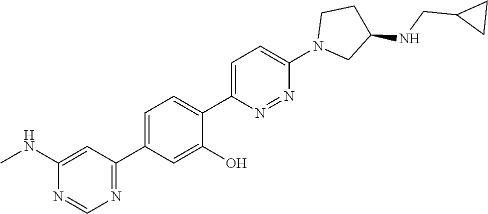 [SMILES: CNc1cc(-c2ccc(-c3ccc(N4CC[C@@H](NCC5CC5)C4)nn3)c(O)c2)ncn1] |
| 498 | 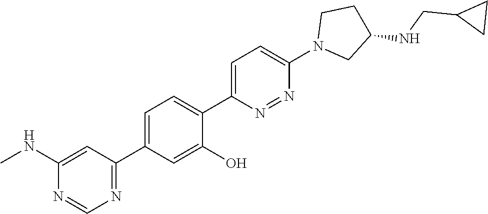 [SMILES: CNc1cc(-c2ccc(-c3ccc(N4CC[C@H](NCC5CC5)C4)nn3)c(O)c2)ncn1] |
| 499 | 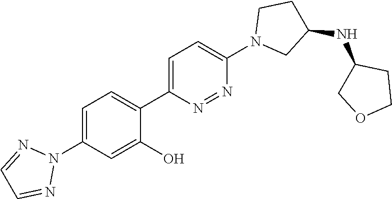 [SMILES: Oc1cc(-n2nccn2)ccc1-c1ccc(N2CC[C@@H](N[C@H]3CCOC3)C2)nn1] |
| 500 | 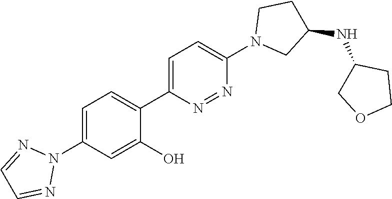 [SMILES: Oc1cc(-n2nccn2)ccc1-c1ccc(N2CC[C@@H](N[C@@H]3CCOC3)C2)nn1] |
| 501 | 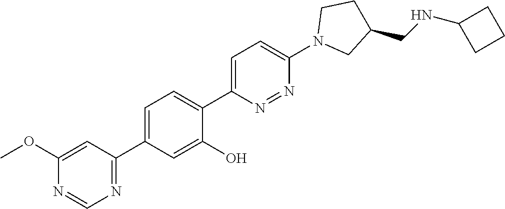 [SMILES: COc1cc(-c2ccc(-c3ccc(N4CC[C@@H](CNC5CCC5)C4)nn3)c(O)c2)ncn1] |
| 502 | 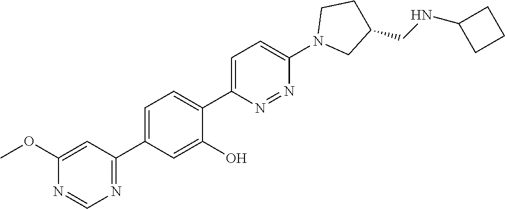 [SMILES: COc1cc(-c2ccc(-c3ccc(N4CC[C@H](CNC5CCC5)C4)nn3)c(O)c2)ncn1] |
| 503 | 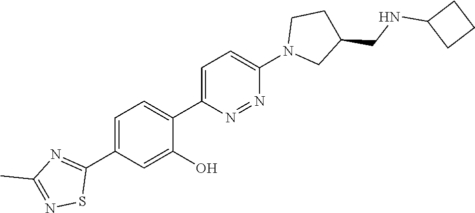 [SMILES: Cc1nsc(-c2ccc(-c3ccc(N4CC[C@@H](CNC5CCC5)C4)nn3)c(O)c2)n1] |
| 504 | 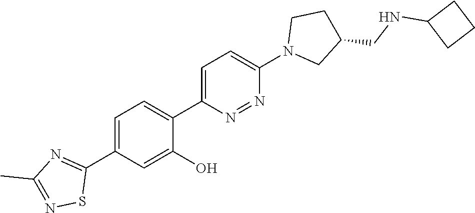 [SMILES: Cc1nsc(-c2ccc(-c3ccc(N4CC[C@H](CNC5CCC5)C4)nn3)c(O)c2)n1] |
| 505 | 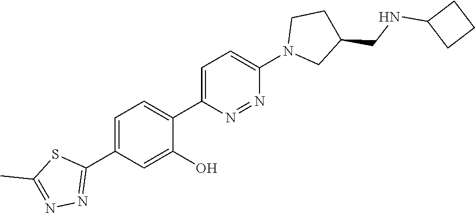 [SMILES: Cc1nnc(-c2ccc(-c3ccc(N4CC[C@@H](CNC5CCC5)C4)nn3)c(O)c2)s1] |
| 506 | 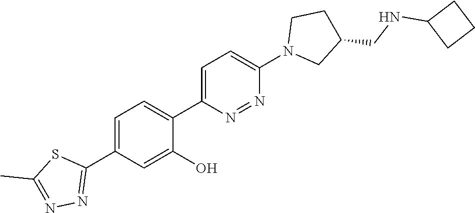 [SMILES: Cc1nnc(-c2ccc(-c3ccc(N4CC[C@H](CNC5CCC5)C4)nn3)c(O)c2)s1] |
| 507 | 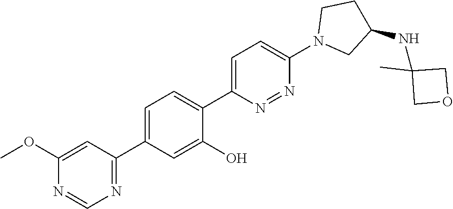 [SMILES: COc1cc(-c2ccc(-c3ccc(N4CC[C@@H](NC5(C)COC5)C4)nn3)c(O)c2)ncn1] |
| 508 | 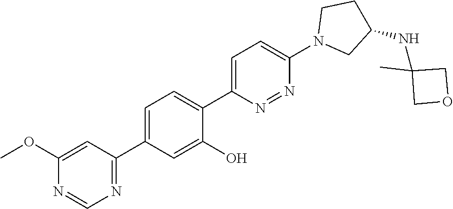 [SMILES: COc1cc(-c2ccc(-c3ccc(N4CC[C@H](NC5(C)COC5)C4)nn3)c(O)c2)ncn1] |
| 509 | 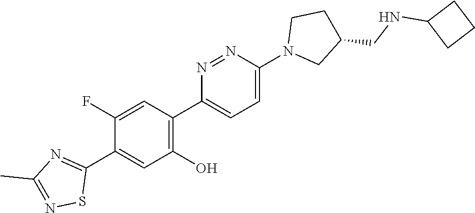 [SMILES: Cc1nsc(-c2cc(O)c(-c3ccc(N4CC[C@H](CNC5CCC5)C4)nn3)cc2F)n1] |
| 510 | 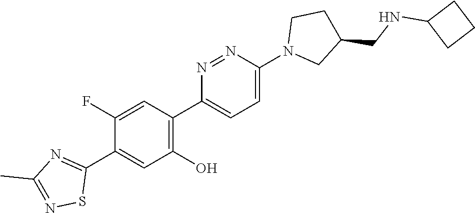 [SMILES: Cc1nsc(-c2cc(O)c(-c3ccc(N4CC[C@@H](CNC5CCC5)C4)nn3)cc2F)n1] |
| 511 | 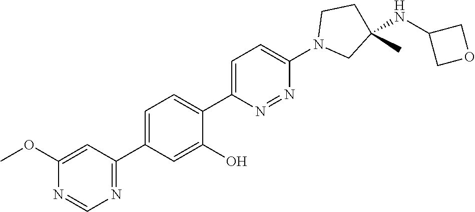 [SMILES: COc1cc(-c2ccc(-c3ccc(N4CC[C@](C)(NC5COC5)C4)nn3)c(O)c2)ncn1] |
| 512 | 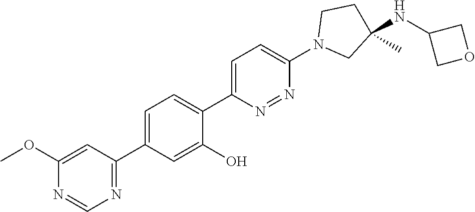 [SMILES: COc1cc(-c2ccc(-c3ccc(N4CC[C@@](C)(NC5COC5)C4)nn3)c(O)c2)ncn1] |
| 513 | 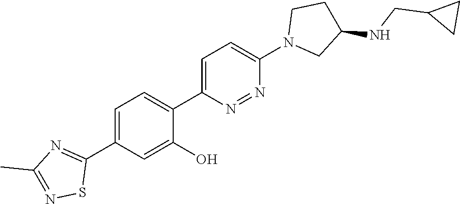 [SMILES: Cc1nsc(-c2ccc(-c3ccc(N4CC[C@@H](NCC5CC5)C4)nn3)c(O)c2)n1] |
| 514 | 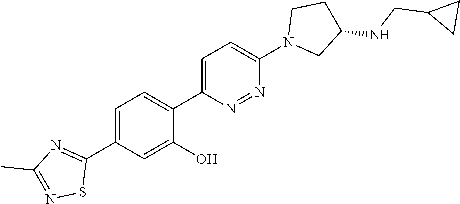 [SMILES: Cc1nsc(-c2ccc(-c3ccc(N4CC[C@H](NCC5CC5)C4)nn3)c(O)c2)n1] |
| 515 | 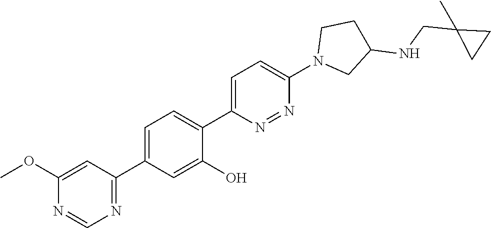 [SMILES: COc1cc(-c2ccc(-c3ccc(N4CCC(NCC5(C)CC5)C4)nn3)c(O)c2)ncn1] |
| 516 | 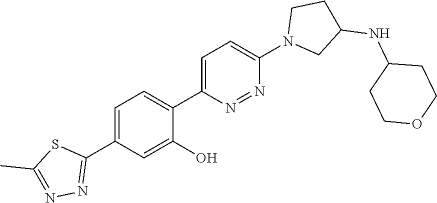 [SMILES: Cc1nnc(-c2ccc(-c3ccc(N4CCC(NC5CCOCC5)C4)nn3)c(O)c2)s1] |
| 517 | 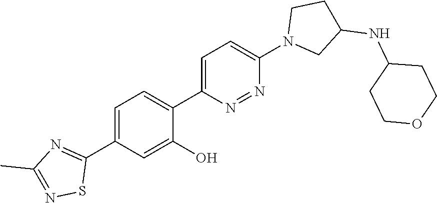 [SMILES: Cc1nsc(-c2ccc(-c3ccc(N4CCC(NC5CCOCC5)C4)nn3)c(O)c2)n1] |
| 518 | 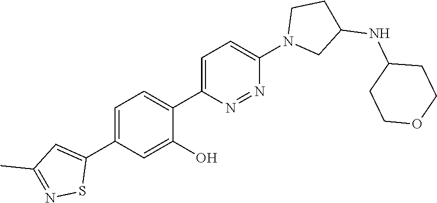 [SMILES: Cc1cc(-c2ccc(-c3ccc(N4CCC(NC5CCOCC5)C4)nn3)c(O)c2)sn1] |
| 519 | 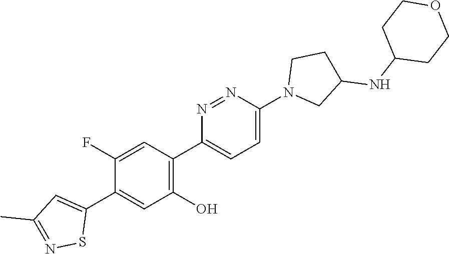 [SMILES: Cc1cc(-c2cc(O)c(-c3ccc(N4CCC(NC5CCOCC5)C4)nn3)cc2F)sn1] |
| 520 | 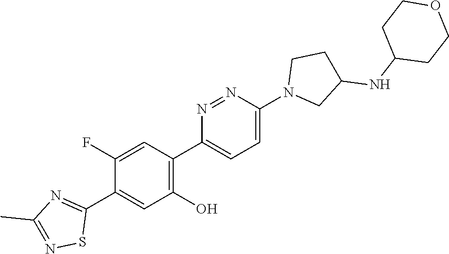 [SMILES: Cc1nsc(-c2cc(O)c(-c3ccc(N4CCC(NC5CCOCC5)C4)nn3)cc2F)n1] |
| 521 | 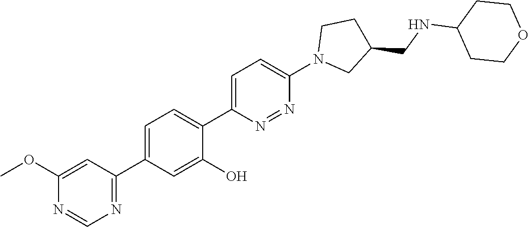 [SMILES: COc1cc(-c2ccc(-c3ccc(N4CC[C@@H](CNC5CCOCC5)C4)nn3)c(O)c2)ncn1] |
| 522 | 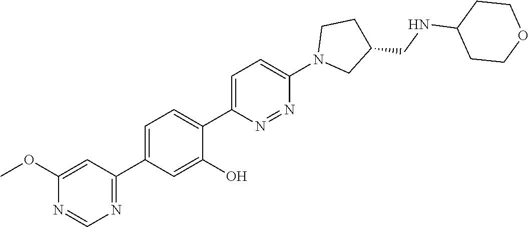 [SMILES: COc1cc(-c2ccc(-c3ccc(N4CC[C@H](CNC5CCOCC5)C4)nn3)c(O)c2)ncn1] |
| 523 | 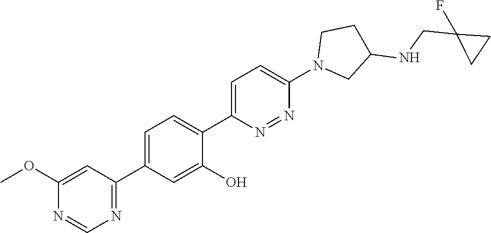 [SMILES: COc1cc(-c2ccc(-c3ccc(N4CCC(NCC5(F)CC5)C4)nn3)c(O)c2)ncn1] |
| 524 | 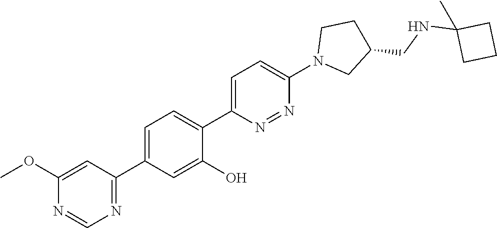 [SMILES: COc1cc(-c2ccc(-c3ccc(N4CC[C@H](CNC5(C)CCC5)C4)nn3)c(O)c2)ncn1] |
| 525 | 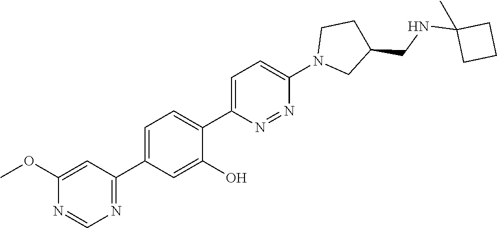 [SMILES: COc1cc(-c2ccc(-c3ccc(N4CC[C@@H](CNC5(C)CCC5)C4)nn3)c(O)c2)ncn1] |
| 526 | 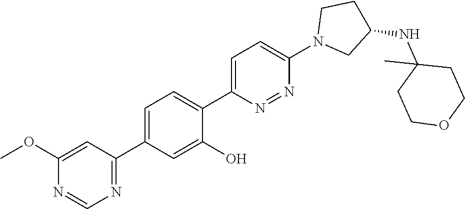 [SMILES: COc1cc(-c2ccc(-c3ccc(N4CC[C@H](NC5(C)CCOCC5)C4)nn3)c(O)c2)ncn1] |
| 527 | 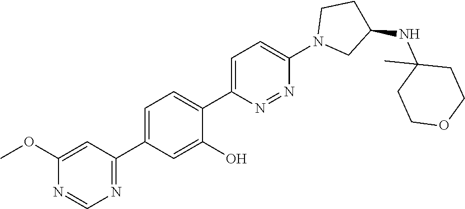 [SMILES: COc1cc(-c2ccc(-c3ccc(N4CC[C@@H](NC5(C)CCOCC5)C4)nn3)c(O)c2)ncn1] |
| 528 | 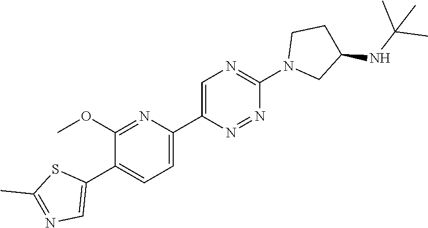 [SMILES: COc1nc(-c2cnc(N3CC[C@@H](NC(C)(C)C)C3)nn2)ccc1-c1cnc(C)s1] |
| 529 | 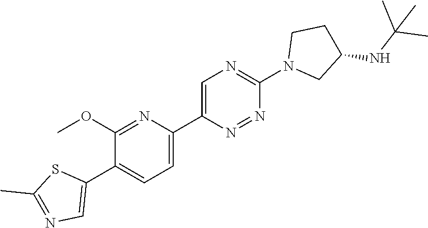 [SMILES: COc1nc(-c2cnc(N3CC[C@H](NC(C)(C)C)C3)nn2)ccc1-c1cnc(C)s1] |
| 530 | 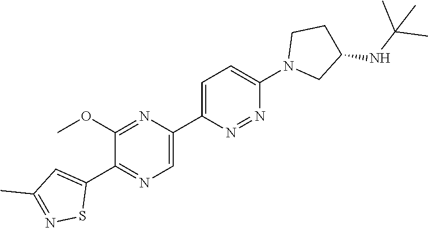 [SMILES: COc1nc(-c2ccc(N3CC[C@H](NC(C)(C)C)C3)nn2)cnc1-c1cc(C)ns1] |
| 531 | 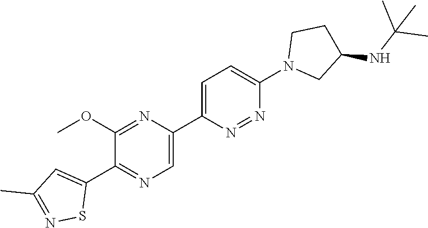 [SMILES: COc1nc(-c2ccc(N3CC[C@@H](NC(C)(C)C)C3)nn2)cnc1-c1cc(C)ns1] |
| 532 | 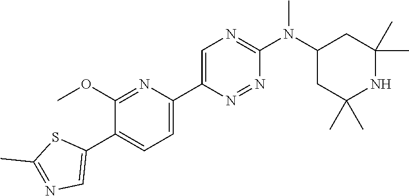 |
| 533 | 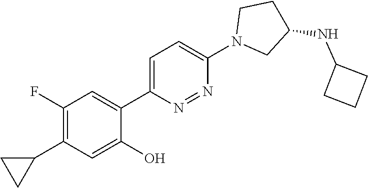 [SMILES: Oc1cc(C2CC2)c(F)cc1-c1ccc(N2CC[C@H](NC3CCC3)C2)nn1] |
| 534 | 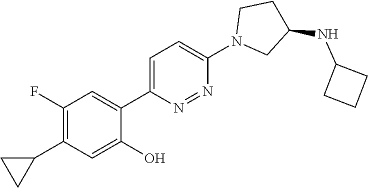 [SMILES: Oc1cc(C2CC2)c(F)cc1-c1ccc(N2CC[C@@H](NC3CCC3)C2)nn1] |
| 535 | 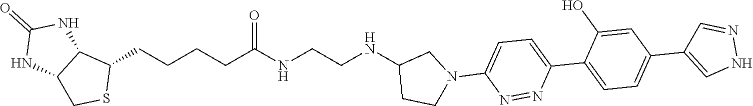 [SMILES: O=C(CCCC[C@@H]1SC[C@@H]2NC(=O)N[C@@H]21)NCCNC1CCN(c2ccc(-c3ccc(-c4cn[nH]c4)cc3O)nn2)C1] |
| 536 | 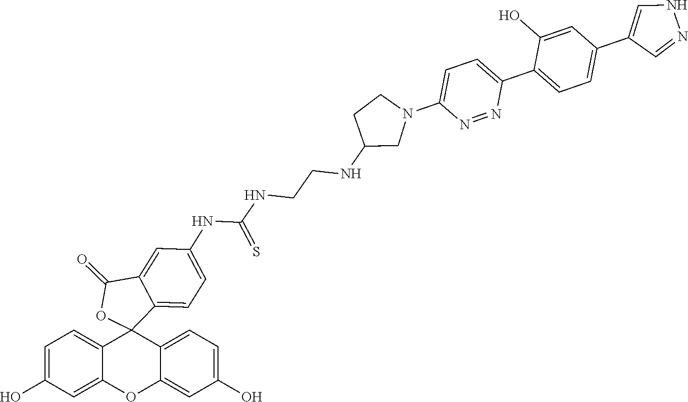 [SMILES: O=C1OC2(c3ccc(O)cc3Oc3cc(O)ccc32)c2ccc(NC(=S)NCCNC3CCN(c4ccc(-c5ccc(-c6cn[nH]c6)cc5O)nn4)C3)cc21] |
| 537 | 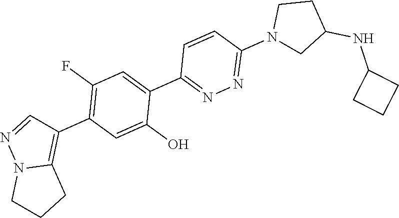 [SMILES: Oc1cc(-c2cnn3c2CCC3)c(F)cc1-c1ccc(N2CCC(NC3CCC3)C2)nn1] |

In some embodiments, for Formula (I), A is monocyclic heteroaryl (e.g., pyrazolyl); L is absent; X is N; R^{4a} is cycloalkyl (e.g., cyclopropyl); R^{4b} is hydrogen; R⁶ is hydrogen; m is 0; n is 0; and p is 2. In some embodiments, the compound of Formulas (I), (I-a), (I-b), (I-c) and (I-e) is Compound 100, 103, 104, or a pharmaceutically acceptable salt, solvate, hydrate, tautomer, or stereoisomer thereof.

In some embodiments, for Formula (I), A is monocyclic heteroaryl (e.g., pyrazolyl); L is absent; X is N; R^{4a} is C₁-C₆-alkyl (e.g., tert-butyl); R^{4b} is hydrogen; R6 is hydrogen; m is 0; n is 0; and p is 2. In some embodiments, the compound of Formulas (I), (I-a), (I-b), (I-c) and (I-e) is Compound 101, or a pharmaceutically acceptable salt, solvate, hydrate, tautomer, or stereoisomer thereof.

In some embodiments, for Formula (I), A is monocyclic heteroaryl (e.g., pyrazolyl); L is absent; X is N; R² is halo (e.g., fluoro); R^{4a} is cycloalkyl (e.g., cyclopropyl); R^{4b} is hydrogen; R⁶ is hydrogen; m is 1; n is 0; and p is 2. In some embodiments, the compound of Formulas (I), (I-a), (I-b), (I-c) and (I-e) is Compound 105, 106, 243, or a pharmaceutically acceptable salt, solvate, hydrate, tautomer, or stereoisomer thereof.

In some embodiments, for Formula (I), A is monocyclic heteroaryl (e.g., pyrazolyl); L is absent; X is N; R^{4a} is cycloalkyl (e.g., 1-methylcyclopropyl); R^{4b} is hydrogen; R⁶ is hydrogen; m is 0; n is 0; and p is 2. In some embodiments, the compound of Formulas (I), (I-a) (I-b), (I-c) and (I-e) is Compound 107, or a pharmaceutically acceptable salt, solvate, hydrate, tautomer, or stereoisomer thereof.

In some embodiments, for Formula (I), A is monocyclic heteroaryl (e.g., pyrazolyl); L is absent; X is N; R^{4a} is C₁-C₆-alkyl (e.g., cyclopropylmethyl); R^{4b} is hydrogen; R⁶ is hydrogen; m is 0; n is 0; and p is 2. In some embodiments, the compound of Formulas (I), (I-a)(I-b), (I-c) and (I-e)) is Compound 108, or a pharmaceutically acceptable salt, solvate, hydrate, tautomer, or stereoisomer thereof.

In some embodiments, for Formula (I), A is monocyclic heteroaryl (e.g., pyrazolyl); L is absent; X is N; R^{4a} is cycloalkyl (e.g., 1-(trifluoromethyl)cyclopropyl); R^{4b} is hydrogen; R⁶ is hydrogen; m is 0; n is 0; and p is 2. In some embodiments, the compound of Formulas (I), (I-b), (I-c) and (I-e) is Compound 110, or a pharmaceutically acceptable salt, solvate, hydrate, tautomer, or stereoisomer thereof.

In some embodiments, for Formula (I), A is monocyclic heteroaryl (e.g., pyrazolyl); L is absent; X is N; R² is halo (e.g., fluoro); R^{4a} is cycloalkyl (e.g., 1-methylcyclopropyl); R^{4b} is hydrogen; R⁶ is hydrogen; m is 1; n is 0; and p is 2. In some embodiments, the compound of Formulas (I), (I-b), (I-c) and (I-e) is Compound 111, or a pharmaceutically acceptable salt, solvate, hydrate, tautomer, or stereoisomer thereof.

In some embodiments, for Formula (I), A is monocyclic heteroaryl (e.g., pyrazolyl); L is absent; X is N; R^{4a} is cycloalkyl (e.g., 1-(fluoromethyl)cyclopropyl); R^{4b} is hydrogen; R⁶ is hydrogen; m is 0; n is 0; and p is 2. In some embodiments, the compound of Formulas (I), (I-b), (I-c) and (I-e) is Compound 112, or a pharmaceutically acceptable salt, solvate, hydrate, tautomer, or stereoisomer thereof.

In some embodiments, for Formula (I), A is monocyclic heteroaryl (e.g., pyrazolyl); L is absent; X is N; R² is halo (e.g., fluoro); R^{4a} is cycloalkyl (e.g., cyclobutyl); R^{4b} is hydrogen; R⁶ is hydrogen; m is 1; n is 0; and p is 2. In some embodiments, the compound of Formulas (I), (I-b), (I-c) and (I-e) is Compound 113, 115, 244, or a pharmaceutically acceptable salt, solvate, hydrate, tautomer, or stereoisomer thereof.

In some embodiments, for Formula (I), A is monocyclic heteroaryl (e.g., pyrazolyl); L is absent; X is N; R^{4a} is cycloalkyl (e.g., cyclobutyl); R^{4b} is hydrogen; R⁶ is hydrogen; m is 0; n is 0; and p is 2. In some embodiments, the compound of Formulas (I), (I-b), (I-c) and (I-e) is Compound 114, 116, 245, or a pharmaceutically acceptable salt, solvate, hydrate, tautomer, or stereoisomer thereof.

In some embodiments, for Formula (I), A is monocyclic heteroaryl (e.g., pyrazolyl); L is absent; X is N; R^{4a} is cycloalkyl (e.g., 1-(difluoromethyl)cyclopropyl); R^{4b} is hydrogen; R⁶ is hydrogen; m is 0; n is 0; and p is 2. In some embodiments, the compound of Formulas (I), (I-b), (I-c) and (I-e) is Compound 117, or a pharmaceutically acceptable salt, solvate, hydrate, tautomer, or stereoisomer thereof.

In some embodiments, for Formula (I), A is monocyclic heteroaryl (e.g., pyrazolyl); L is absent; X is N; R² is halo (e.g., fluoro); R^{4a} is C₁-C₆-alkyl (e.g., tert-butyl); R^{4b} is methyl; R⁶ is hydrogen; m is 1; n is 0; and p is 2. In some embodiments, the compound of Formulas (I), (I-a), (I-b), (I-c) and (I-e) is Compound 118, 119, 120, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R^{4a} is cycloalkyl (e.g., 1-methylcyclopropyl); R^{4b} is hydrogen; R⁶ is hydrogen; m is 0; n is 0; and p is 2. In some embodiments, the compound of Formulas (I), (I-a), (I-b), (I-c) and (I-e) is Compound 124, 125, 126, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R^{4a} is cycloalkyl (e.g., 1-(fluoromethyl)cyclopropyl); R^{4b}is hydrogen; R⁶ is hydrogen; m is 0; n is 0; and p is 2. In some embodiments, the compound of Formulas (I), (I-a), (I-b), (I-c), (I-e), and (I-f) is Compound 127, 128, 129, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R^{4a} is cycloalkyl (e.g., 1-methylcyclobutyl); R^{4b} is hydrogen; R⁶ is hydrogen; m is 0; n is 0; and p is 2. In some embodiments, the compound of Formulas (I), (I-a), (I-b), (I-c), (I-e), and (I-f) is Compound 130, 131, 132, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R^{4a} is C₁-C₆-alkyl (e.g., cyclopropylmethyl); R^{4b} is hydrogen; R⁶ is hydrogen; m is 0; n is 0; and p is 2. In some embodiments, the compound of Formulas (I), (I-a), (I-b), (I-c), (I-e), and (I-f) is Compound 133, 134, 135, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R^{4a} is cycloalkyl (e.g., 3-fluorocyclobutyl); R^{4b} is hydrogen; R⁶ is hydrogen; m is 0; n is 0; and p is 2. In some embodiments, the compound of Formulas (I), (I-a), (I-b), (I-c), (I-e), (I-f) is Compound 136, 137, 138, 139, 140, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R^{4a} is cycloalkyl (e.g., cyclobutyl); R^{4b} is hydrogen; R⁶ is hydrogen; m is 0; n is 0; and p is 2. In some embodiments, the compound of Formulas (I), (I-a), (I-b), (I-c) and (I-e) is Compound 141, 142, 143, or a pharmaceutically acceptable salt, solvate, hydrate, tautomer, or stereoisomer thereof.

In some embodiments, for Formula (I), A is monocyclic heteroaryl (e.g., 2-methylthiazolyl); L is absent; X is N; R² is halo (e.g., fluoro); R^{4a} is cycloalkyl (e.g., cyclobutyl); R^{4b} is hydrogen; R⁶ is hydrogen; m is 1; n is 0; and p is 2. In some embodiments, the compound of Formulas (I), (I-a), (I-b), (I-c), (I-e), and (I-g) is Compound 144, 145, 146, or a pharmaceutically acceptable salt, solvate, hydrate, tautomer, or stereoisomer thereof.

In some embodiments, for Formula (I), A is monocyclic heteroaryl (e.g., 2-methylthiazolyl); L is absent; X is N; R^{4a} is cycloalkyl (e.g., cyclobutyl); R^{4b} is hydrogen; R⁶ is hydrogen; m is 0; n is 0; and p is 2. In some embodiments, the compound of Formulas (I), (I-a), (I-b), (I-c), (I-e), and (I-f) is Compound 147, 148, 149, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R² is halo (e.g., fluoro); R^{4a} is cycloalkyl (e.g., 1-methylcyclopropyl); R^{4b} is hydrogen; R⁶ is hydrogen; m is 1; n is 0; and p is 2. In some embodiments, the compound of Formulas (I), (I-a), (I-b), (I-c), (I-e), and (I-f) is Compound 154, 155, 156, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R² is halo (e.g., fluoro); R^{4a} is cycloalkyl (e.g., 1-methylcyclobutyl); R^{4b} is hydrogen; R⁶ is hydrogen; m is 1; n is 0; and p is 2. In some embodiments, the compound of Formulas (I), (I-a), (I-b), (I-c), (I-e), and (I-f) is Compound 157, 158, 159, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R² is halo (e.g., fluoro); R^{4a} is cycloalkyl (e.g., 3-fluorocyclopropyl); R^{4b} is hydrogen; R⁶ is hydrogen; m is 1; n is 0; and p is 2. In some embodiments, the compound of Formulas (I), (I-a), (I-b), (I-c), (I-e), and (I-f) is Compound 160, 161, 162, 257, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R² is halo (e.g., fluoro); R^{4a} is C₁-C₆-alkyl (e.g., cyclopropylmethyl); R^{4b} is hydrogen; R⁶ is hydrogen; m is 1; n is 0; and p is 2. In some embodiments, the compound of Formulas (I), (I-a), (I-b), (I-c), (I-e), and (I-f) is Compound 163, 164, 165, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R² is halo (e.g., fluoro); R^{4a} is cycloalkyl (e.g., 1-fluoromethylcyclopropyl); R^{4b} is hydrogen; R⁶ is hydrogen; m is 1; n is 0; and p is 2. In some embodiments, the compound of Formulas (I), (I-a), (I-b), (I-c), (I-e), and (I-f) is Compound 166, 167, 168, or a pharmaceutically acceptable salt, solvate, hydrate, tautomer, or stereoisomer thereof.

In some embodiments, for Formula (I), A is monocyclic heteroaryl (e.g., 2-methyl-1,3,4-thiadiazolyl); L is absent; X is N; R^{4a} is cycloalkyl (e.g., cyclobutyl); R^{4b} is hydrogen; R⁶ is hydrogen; m is 0; n is 0; and p is 2. In some embodiments, the compound of Formulas (I), (I-a), (I-b), (I-c), (I-e), and (I-g) is Compound 172, 173, 174, or a pharmaceutically acceptable salt, solvate, hydrate, tautomer, or stereoisomer thereof.

In some embodiments, for Formula (I), A is monocyclic heteroaryl (e.g., 2-methyl-1,3,4-thiadiazolyl); L is absent; X is N; R² is halo (e.g., fluoro); R^{4a} is cycloalkyl (e.g., cyclobutyl); R^{4b}is hydrogen; R⁶ is hydrogen; m is 1; n is 0; and p is 2. In some embodiments, the compound of Formulas (I), (I-a), (I-b), (I-c), (I-e), and (I-g) is Compound 175, 176, 177, or a pharmaceutically acceptable salt, solvate, hydrate, tautomer, or stereoisomer thereof.

In some embodiments, for Formula (I), A is monocyclic heteroaryl (e.g., 2-methoxy-1,3,4-thiadiazolyl); L is absent; X is N; R² is halo (e.g., fluoro); R^{4a} is cycloalkyl (e.g., cyclobutyl); R^{4b} is hydrogen; R⁶ is hydrogen; m is 1; n is 0; and p is 2. In some embodiments, the compound of Formulas (I), (I-a), (I-b), (I-c), (I-e), and (I-g) is Compound 178, 179, 180, or a pharmaceutically acceptable salt, solvate, hydrate, tautomer, or stereoisomer thereof.

In some embodiments, for Formula (I), A is monocyclic heteroaryl (e.g., 2-methoxythiazolyl); L is absent; X is N; R² is halo (e.g., fluoro); R^{4a} is cycloalkyl (e.g., cyclobutyl); R^{4b} is hydrogen; R⁶ is hydrogen; m is 1; n is 0; and p is 2. In some embodiments, the compound of Formulas (I), (I-a), (I-b), (I-c), (I-e), and (I-fg is Compound 181, 182, 183, or a pharmaceutically acceptable salt, solvate, hydrate, tautomer, or stereoisomer thereof.

In some embodiments, for Formula (I), A is monocyclic heteroaryl (e.g., 2-methoxythiazolyl); L is absent; X is N; R^{4a} is cycloalkyl (e.g., cyclobutyl); R^{4b} is hydrogen; R⁶ is hydrogen; m is 0; n is 0; and p is 2. In some embodiments, the compound of Formulas (I), (I-a), (I-b), (I-c), (I-e), and (I-g) is Compound 184, 185, 186, or a pharmaceutically acceptable salt, solvate, hydrate, tautomer, or stereoisomer thereof.

In some embodiments, for Formula (I), A is monocyclic heteroaryl (e.g., 2-methylthiazolyl); L is absent; X is N; R^{4a} is cycloalkyl (e.g., 1-methylcyclobutyl); R^{4b} is hydrogen; R⁶ is hydrogen; m is 0; n is 0; and p is 2. In some embodiments, the compound of Formulas (I), (I-a), (I-b), (I-c), (I-e), and (I-g) is Compound 187, 188, 189, or a pharmaceutically acceptable salt, solvate, hydrate, tautomer, or stereoisomer thereof.

In some embodiments, for Formula (I), A is monocyclic heteroaryl (e.g., 2-methylthiazolyl); L is absent; X is N; R² is halo (e.g., fluoro); R^{4a} is cycloalkyl (e.g., 1-methylcyclobutyl); R^{4b} is hydrogen; R⁶ is hydrogen; m is 1; n is 0; and p is 2. In some embodiments, the compound of Formulas (I), (I-a), (I-b), (I-c), (I-e), and (I-g) is Compound 190, 191, 192, or a pharmaceutically acceptable salt, solvate, hydrate, tautomer, or stereoisomer thereof.

In some embodiments, for Formula (I), A is monocyclic heteroaryl (e.g., 2-methylthiazolyl); L is absent; X is N; R² is halo (e.g., fluoro); R^{4a} is cycloalkyl (e.g., 3-fluorocyclobutyl); R^{4b} is hydrogen; R⁶ is hydrogen; m is 1; n is 0; and p is 2. In some embodiments, the compound of Formulas (I), (I-a), (I-b), (I-c), (I-e), and (I-g) is Compound 193, 194, 195, 196, 197, or a pharmaceutically acceptable salt, solvate, hydrate, tautomer, or stereoisomer thereof.

In some embodiments, for Formula (I), A is monocyclic heteroaryl (e.g., 2-methylthiazolyl); L is absent; X is N; R^{4a} is cycloalkyl (e.g., 3-fluorocyclobutyl); R^{4b} is hydrogen; R⁶ is hydrogen; m is 0; n is 0; and p is 2. In some embodiments, the compound of Formulas (I), (I-a), (I-b), (I-c), (I-e), and (I-g) is Compound 198, 199, 200, 201, A or a pharmaceutically acceptable salt, solvate, hydrate, tautomer, or stereoisomer thereof.

In some embodiments, for Formula (I), A is monocyclic heteroaryl (e.g., pyrazolyl); L is absent; X is N; R^{4a} is C₁-C₆-heteroalkyl (e.g., 2-aminoethyl); R^{4b} is hydrogen; R⁶ is hydrogen; m is 0; n is 0; and p is 2. In some embodiments, the compound of Formulas (I), (I-a), (I-b), (I-c), and (I-e), is Compound 208, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R^{4a} is C₁-C₆-alkyl (e.g., tert-butyl); R^{4b} is hydrogen; R⁶ is hydrogen; m is 0; n is 0; and p is 2. In some embodiments, the compound of Formulas (I), (I-a), (I-b), (I-c), (I-e), and (I-f) is Compound 209, 210, 246, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R² is halo (e.g., fluoro); R^{4a} is C₁-C₆-alkyl (e.g., tert-butyl); R^{4b} is hydrogen; R⁶ is hydrogen; m is 1; n is 0; and p is 2. In some embodiments, the compound of Formulas (I), (I-a), (I-b), (I-c), (I-e), and (I-f) is Compound 211, 212, 247, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R² is halo (e.g., fluoro); R^{4a} is cycloalkyl (e.g., cyclopropyl); R^{4b} is hydrogen; R⁶ is hydrogen; m is 1; n is 0; and p is 2. In some embodiments, the compound of Formulas (I), (I-a), (I-b), (I-c), (I-e), and (I-f) is Compound 213, 214, 248, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R^{4a} is cycloalkyl (e.g., cyclopropyl); R^{4b} is hydrogen; R⁶ is hydrogen; m is 0; n is 0; and p is 2. In some embodiments, the compound of Formulas (I), (I-a), (I-b), (I-c), (I-e), and (I-f) is Compound 215, 216, 249, or a pharmaceutically acceptable salt, solvate, hydrate, tautomer, or stereoisomer thereof.

In some embodiments, for Formula (I), A is monocyclic heteroaryl (e.g., pyridin-2(1H)-only); L is absent; X is N; R² is halo (e.g., fluoro); R^{4a} is cycloalkyl (e.g., cyclobutyl); R^{4b} is hydrogen; R⁶ is hydrogen; m is 1; n is 0; and p is 2. In some embodiments, the compound of Formulas (I), (I-a), (I-b), (I-c), and (I-e), is Compound 220, 221, 251, or a pharmaceutically acceptable salt, solvate, hydrate, tautomer, or stereoisomer thereof.

In some embodiments, for Formula (I), A is monocyclic heteroaryl (e.g., 2-methylpyridinyl); L is absent; X is N; R² is halo (e.g., fluoro); R^{4a} is cycloalkyl (e.g., cyclobutyl); R^{4b} is hydrogen; R⁶ is hydrogen; m is 1; n is 0; and p is 2. In some embodiments, the compound of Formulas (I), (I-a), (I-b), (I-c), and (I-e), is Compound 222, 223, 252, or a pharmaceutically acceptable salt, solvate, hydrate, tautomer, or stereoisomer thereof.

In some embodiments, for Formula (I), A is monocyclic heteroaryl (e.g., 3-methoxypyridinyl); L is absent; X is N; R² is halo (e.g., fluoro); R^{4a} is cycloalkyl (e.g., cyclobutyl); R^{4b} is hydrogen; R⁶ is hydrogen; m is 1; n is 0; and p is 2. In some embodiments, the compound of Formulas (I), (I-a), (I-b), (I-c), and (I-e), is Compound 224, 225, 253, or a pharmaceutically acceptable salt, solvate, hydrate, tautomer, or stereoisomer thereof.

In some embodiments, for Formula (I), A is monocyclic heteroaryl (e.g., 4-methyl-1H-imidazolyl); L is absent; X is N; R² is halo (e.g., fluoro); R^{4a} is cycloalkyl (e.g., cyclobutyl); R^{4b}is hydrogen; R⁶ is hydrogen; m is 1; n is 0; and p is 2. In some embodiments, the compound of Formulas (I), (I-a), (I-b), (I-c), and (I-e), is Compound 226, 227, 254, or a pharmaceutically acceptable salt, solvate, hydrate, tautomer, or stereoisomer thereof.

In some embodiments, for Formula (I), A is monocyclic heteroaryl (e.g., 2-methyloxazolyl); L is absent; X is N; R² is halo (e.g., fluoro); R^{4a} is cycloalkyl (e.g., cyclobutyl); R^{4b} is hydrogen; R⁶ is hydrogen; m is 1; n is 0; and p is 2. In some embodiments, the compound of Formulas (I), (I-a), (I-b), (I-c), and (I-e), is Compound 228, 229, 255, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R² is halo (e.g., fluoro); R^{4a} is cycloalkyl (e.g., cyclobutyl); R^{4b} is hydrogen; R⁶ is hydrogen; m is 1; n is 0; and p is 2. In some embodiments, the compound of Formulas (I), (I-a), (I-b), and (I-d) is Compound 230, 231, 256, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R² is halo (e.g., fluoro); R^{4a} is cycloalkyl (e.g., 3-fluorocyclobutyl); R^{4b} is hydrogen; R⁶ is hydrogen; m is 1; n is 0; and p is 2. In some embodiments, the compound of Formulas (I), (I-a), (I-b), (I-c), (I-e), and (I-f) is Compound 232, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R² is halo (e.g., fluoro); R^{4a} is C₁-C₆-alkyl (e.g., tert-butyl); R^{4b} is methyl; R⁶ is hydrogen; m is 1; n is 0; and p is 2. In some embodiments, the compound of Formulas (I), (I-a), (I-b), (I-c), (I-e), and (I-f) is Compound 234, 235, 258, or a pharmaceutically acceptable salt, solvate, hydrate, tautomer, or stereoisomer thereof.

In some embodiments, for Formula (I), A is monocyclic heteroaryl (e.g., 3-methylpyrimidin-4(3H)-onyl); L is absent; X is N; R^{4a} is C₁-C₆-alkyl (e.g., tert-butyl); R^{4b} is hydrogen; R⁶ is hydrogen; m is 0; n is 0; and p is 2. In some embodiments, the compound of Formulas (I), (I-a), (I-b), (I-c), and (I-e), is Compound 236, 237, 259, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R^{4a} is cycloalkyl (e.g., cyclobutyl); R^{4b} is C₁-C₆-alkyl (e.g., methyl); R⁶ is hydrogen; m is 0; n is 0; and p is 2. In some embodiments, the compound of Formulas (I), (I-a), (I-b), (I-c), (I-e), and (I-f) is Compound 238, 239, 260, or a pharmaceutically acceptable salt, solvate, hydrate, tautomer, or stereoisomer thereof.

In some embodiments, for Formula (I), A is monocyclic heteroaryl (e.g., 3-methoxypyridazyl); L is absent; X is N; R^{4a} is cycloalkyl (e.g., 1-fluoromethylcyclopropyl); R^{4b}is hydrogen; R⁶ is hydrogen; m is 0; n is 0; and p is 2. In some embodiments, the compound of Formulas (I), (I-a), (I-b), (I-c), (I-e), and (I-f) is Compound 241, 242, or a pharmaceutically acceptable salt, solvate, hydrate, tautomer, or stereoisomer thereof.

In some embodiments the compound of Formula (II) is a compound of Formula (II-a):

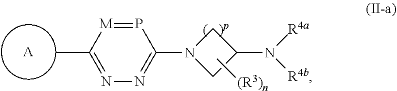

or a pharmaceutically acceptable salt, solvate, hydrate, tautomer, or stereoisomer thereof, wherein A is bicyclic heteroaryl optionally substituted with one or more R¹; M and P are each independently C(R²) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NBC(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NBC(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; and x is 0, 1, or 2.

In some embodiments, the compound of Formula (II) is a compound of Formula (II-b):

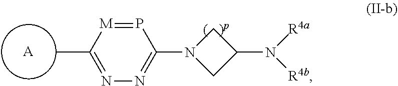

or a pharmaceutically acceptable salt, solvate, hydrate, tautomer, or stereoisomer thereof, wherein A is bicyclic heteroaryl optionally substituted with one or more R¹; M and P are each independently C(R²) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently C₁-C₆-alkyl, C₂-C₆-alkenyl, C₂-C₆-alkynyl, halo, cyano, cycloalkyl, heterocyclyl, or —OR^{A}; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; p is 1, 2, 3, or 4; and x is 0, 1, or 2.

In some embodiments, the compound of Formula (II) is a compound of Formula (II-c):

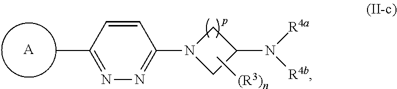

or a pharmaceutically acceptable salt, solvate, hydrate, tautomer, or stereoisomer thereof, wherein A is bicyclic heteroaryl optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, heteroalkyl, haloalkyl, cycloalkyl, heterocyclyl, aryl, and heteroaryl is optionally substituted with one or more R⁶; or R4a and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A}is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; and x is 0, 1, or 2.

In some embodiments, the compound of Formula (II) is a compound of Formula (II-c-i):

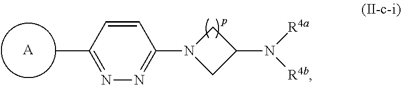

or a pharmaceutically acceptable salt, solvate, hydrate, tautomer, or stereoisomer thereof, wherein A is bicyclic heteroaryl optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; p is 1, 2, 3, or 4; and x is 0, 1, or 2.

In some embodiments, A is selected from A is selected from

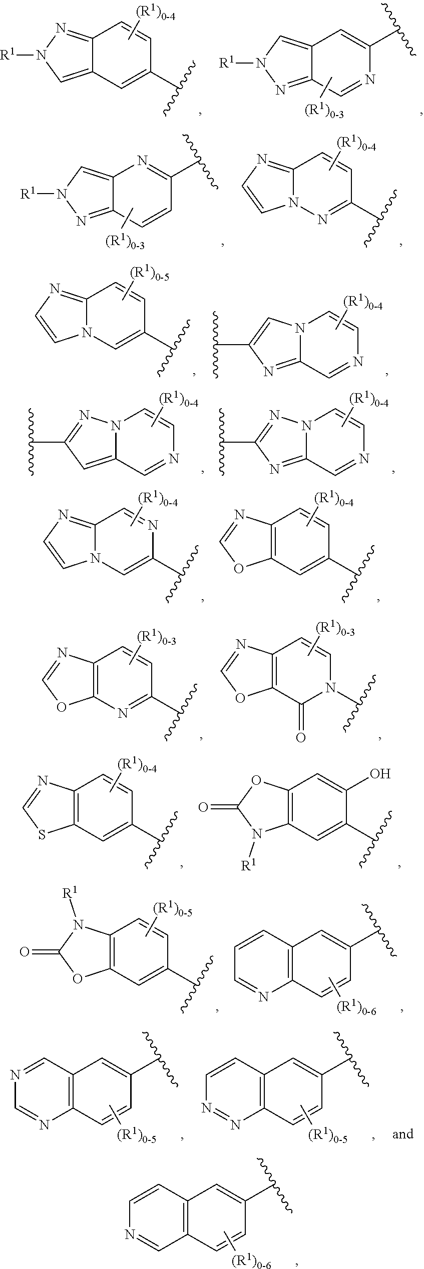

wherein R¹ is as described herein. In some embodiments, A is selected from

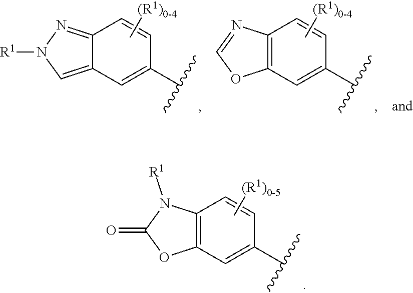

wherein R¹ is as defined herein. In some embodiments, A is selected from

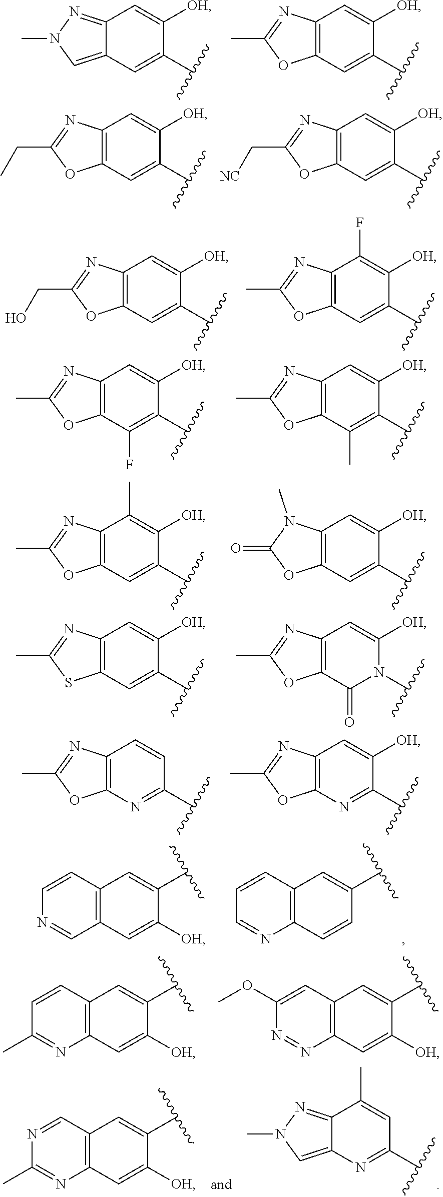

In some embodiments, A is selected from

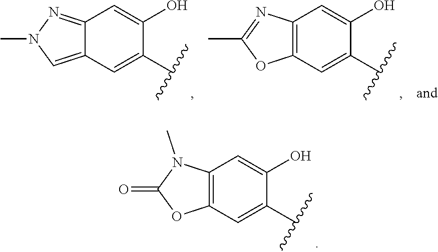 [SMILES: *c1cc2cn(C)nc2cc1O ; *c1cc2oc(C)nc2cc1O ; *c1cc2oc(=O)n(C)c2cc1O]

In some embodiments, each of M and P is independently C(R²), e.g., CH.

In some embodiments, R^{4a} is hydrogen and R^{4b} is t-butyl, cyclopropyl, CH₂-cyclopropyl, cyclobutyl, 1-methylcylcopropyl, 1-trifluoromethylcyclopropyl, 1-fluoromethylcyclopropyl, and 1-difluoromethylcyclopropyl. In some embodiments, p is 1. In some embodiments, p is 2.

In some embodiments,

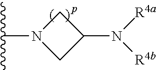

is selected from

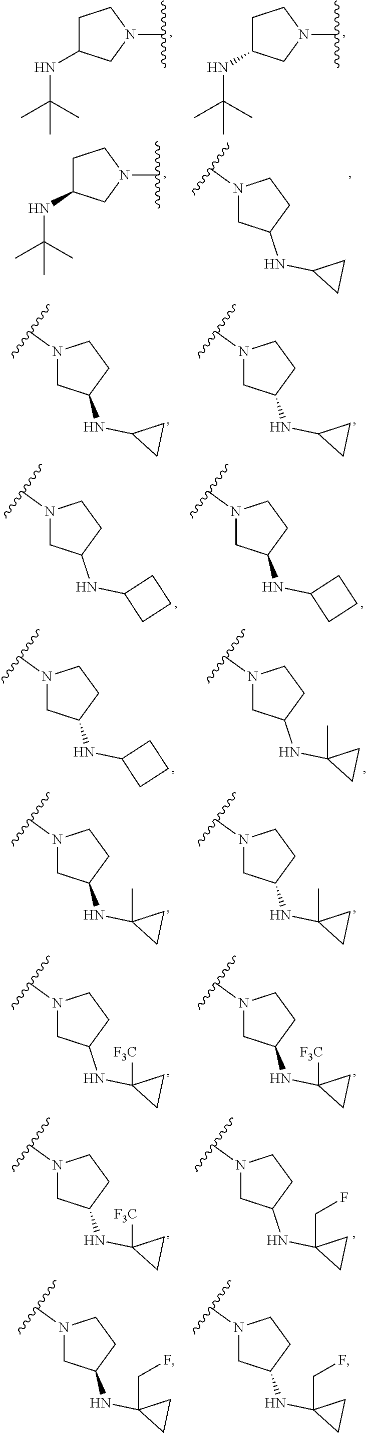

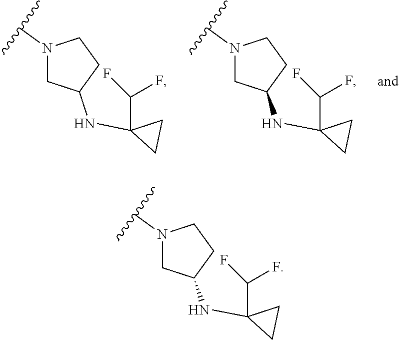 [SMILES: *N1CCC(NC2(C(F)F)CC2)C1 ; *N1CC[C@@H](NC2(C(F)F)CC2)C1 ; *N1CC[C@H](NC2(C(F)F)CC2)C1]

In some embodiments,

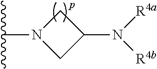

is selected from

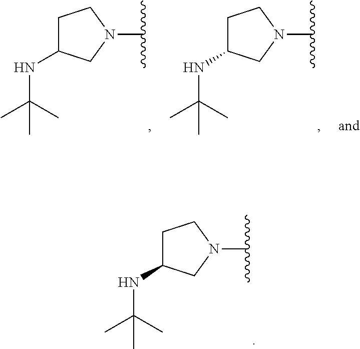 [SMILES: *N1CCC(NC(C)(C)C)C1 ; *N1CC[C@@H](NC(C)(C)C)C1 ; *N1CC[C@H](NC(C)(C)C)C1]

In some embodiments, the compound of Formula (II) is a compound of Formula (II-d):

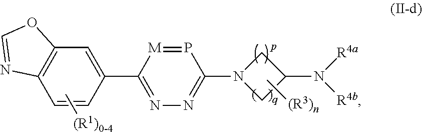

or a pharmaceutically acceptable salt, solvate, hydrate, tautomer, or stereoisomer thereof, wherein M and P are each independently C(R²) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; q is 0, 1, 2, or 3; and x is 0, 1, or 2.

In some embodiments, the compound of Formula (II) is a compound of Formula (II-d-i):

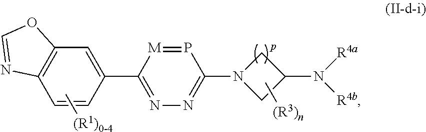

or a pharmaceutically acceptable salt, solvate, hydrate, tautomer, or stereoisomer thereof, wherein M and P are each independently C(R²) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR ^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; and x is 0, 1, or 2.

In some embodiments, p is 1. In some embodiments, p is 2.

In some embodiments, R^{4a} is hydrogen and R^{4b} is t-butyl, cyclopropyl, CH₂-cyclopropyl, cyclobutyl, 1-methylcylcopropyl, 1-trifluoromethylcyclopropyl, 1-fluoromethylcyclopropyl, and 1-difluoromethylcyclopropyl. In some embodiments,

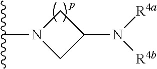

is selected from

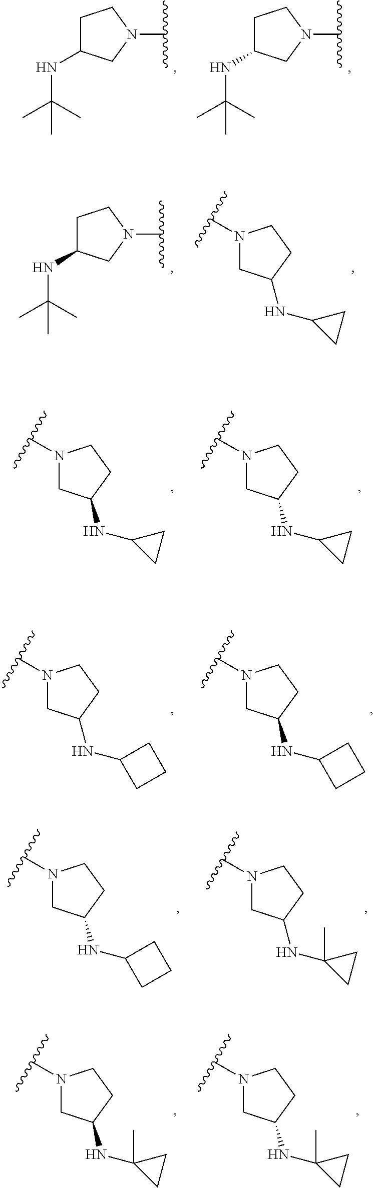

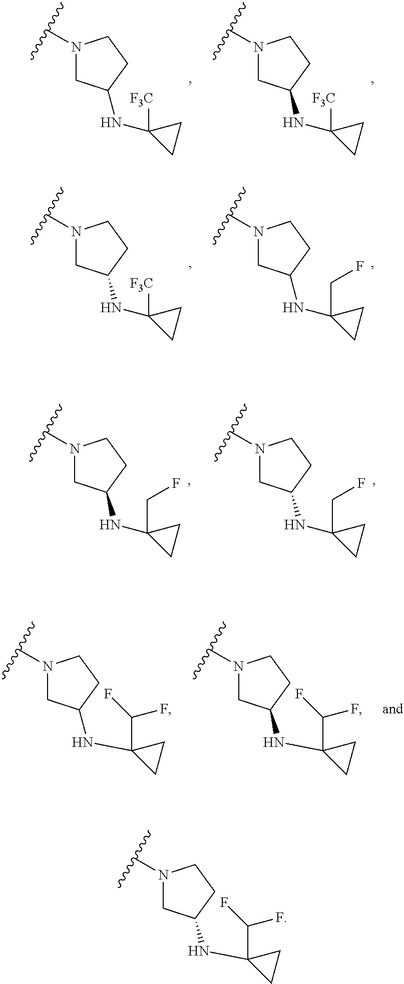

In some embodiments,

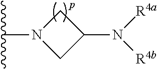

is selected from

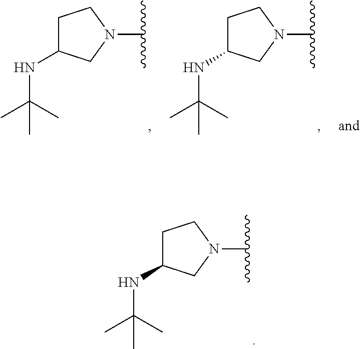 [SMILES: *N1CCC(NC(C)(C)C)C1 ; *N1CC[C@@H](NC(C)(C)C)C1 ; *N1CC[C@H](NC(C)(C)C)C1]

In some embodiments, the compound of Formula (I) is a compound of Formula (I-e):

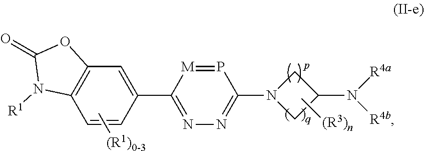

or a pharmaceutically acceptable salt, solvate, hydrate, tautomer, or stereoisomer thereof, wherein M and P are each independently C(R²) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; q is 0, 1, 2, or 3; and x is 0, 1, or 2.

In some embodiments, the compound of Formula (II) is a compound of Formula (II-e-i):

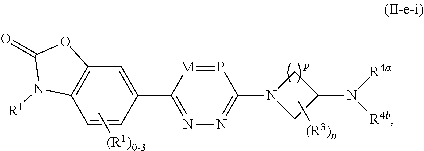

or a pharmaceutically acceptable salt, solvate, hydrate, tautomer, or stereoisomer thereof, wherein M and P are each independently C(R²) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NBC(O)R^{D}—NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; and x is 0, 1, or 2.

In some embodiments, each of M and P is independently C(R²), e.g., CH.

In some embodiments, p is 1. In some embodiments, p is 2.

In some embodiments, R^{4a} is hydrogen and R^{4b} is t-butyl, cyclopropyl, CH₂-cyclopropyl, cyclobutyl, 1-methylcylcopropyl, 1-trifluoromethylcyclopropyl, 1-fluoromethylcyclopropyl, and 1-difluoromethylcyclopropyl. In some embodiments,

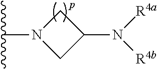

is selected from

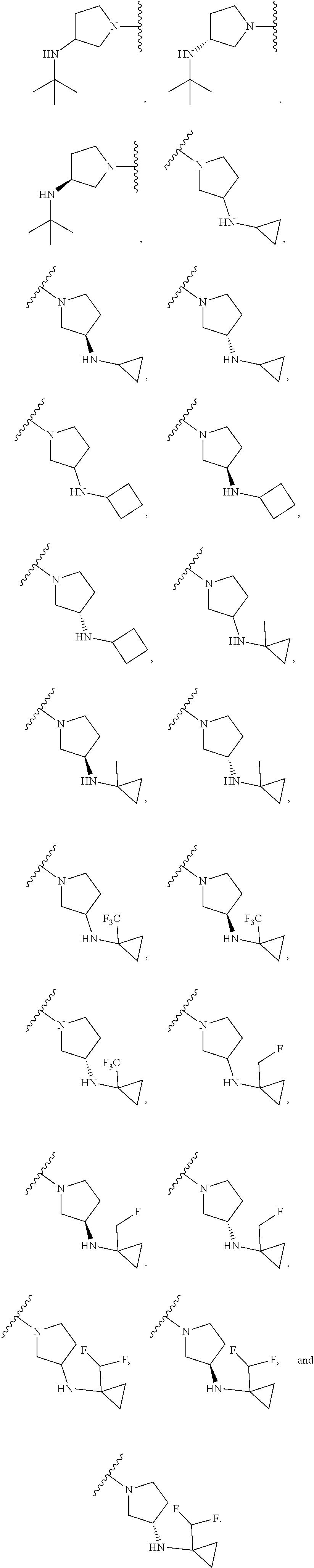

In some embodiments,

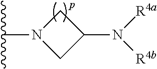

is selected from

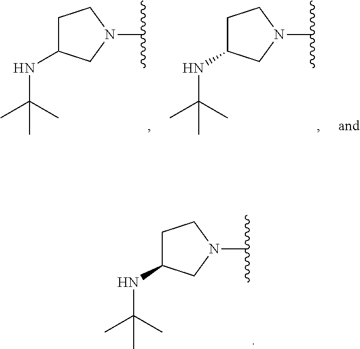 [SMILES: *N1CCC(NC(C)(C)C)C1 ; *N1CC[C@@H](NC(C)(C)C)C1 ; *N1CC[C@H](NC(C)(C)C)C1]

In some embodiments, the compound of Formula (II) is a compound of Formula (II-f):

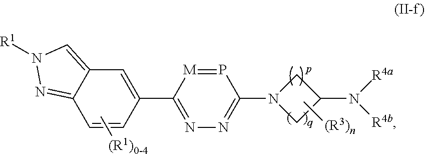

or a pharmaceutically acceptable salt, solvate, hydrate, tautomer, or stereoisomer thereof, wherein M and P are each independently C(R²) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}(O)R^{D}—NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; q is 0, 1, 2, or 3; and x is 0, 1, or 2.

In some embodiments, the compound of Formula (II) is a compound of Formula (II-f-i):

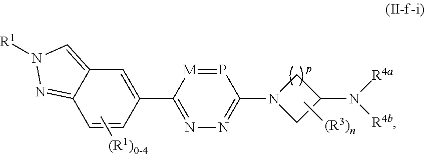

or a pharmaceutically acceptable salt, solvate, hydrate, tautomer, or stereoisomer thereof, wherein M and P are each independently C(R²) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; and x is 0, 1, or 2.

In some embodiments, each of M and P is independently C(R²), e.g., CH.

In some embodiments, p is 1. In some embodiments, p is 2.

In some embodiments, R^{4a} is hydrogen and R^{4b} is t-butyl, cyclopropyl, CH₂-cyclopropyl, cyclobutyl, 1-methylcylcopropyl, 1-trifluoromethylcyclopropyl, 1-fluoromethylcyclopropyl, and 1-difluoromethylcyclopropyl. In some embodiments,

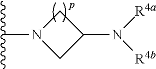

is selected from

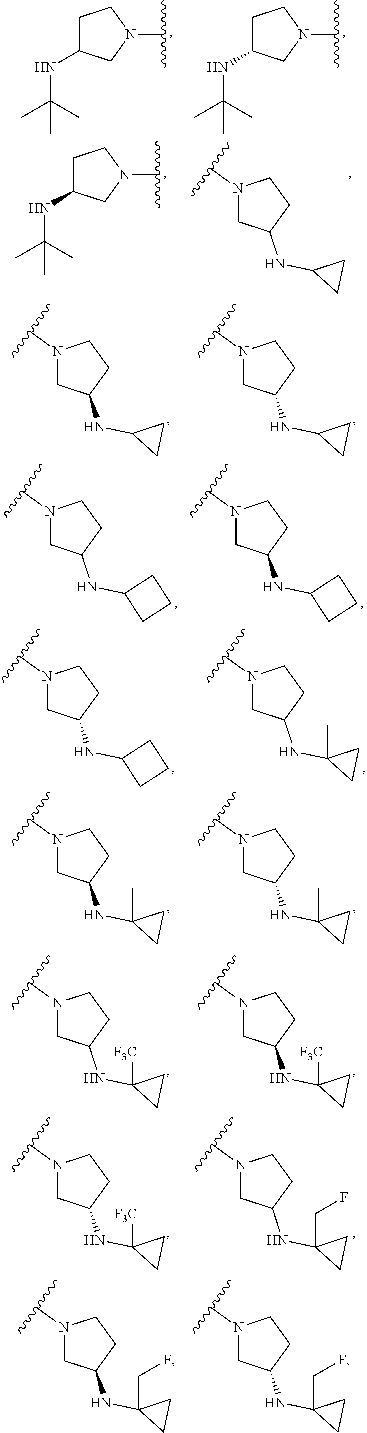

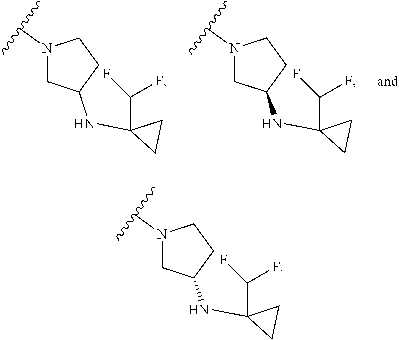 [SMILES: *N1CCC(NC2(C(F)F)CC2)C1 ; *N1CC[C@@H](NC2(C(F)F)CC2)C1 ; *N1CC[C@H](NC2(C(F)F)CC2)C1]

In some embodiments,

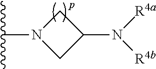

is selected from

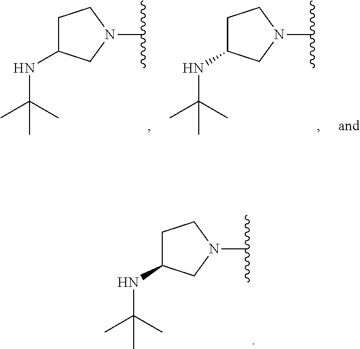 [SMILES: *N1CCC(NC(C)(C)C)C1 ; *N1CC[C@@H](NC(C)(C)C)C1 ; *N1CC[C@H](NC(C)(C)C)C1]

In some embodiments, the compound of Formula (II) is a compound of Formula (II-g):

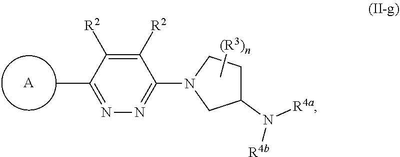

or a pharmaceutically acceptable salt, solvate, hydrate, tautomer, or stereoisomer thereof, wherein A is bicyclic heteroaryl optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —BC(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; and x is 0, 1, or 2.

In some embodiments, the compound of Formula (II) a compound of Formula (II-g-i):

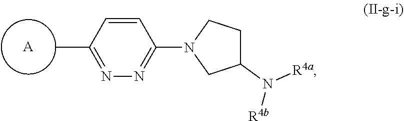

or a pharmaceutically acceptable salt, solvate, hydrate, tautomer, or stereoisomer thereof, wherein A is bicyclic heteroaryl optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; p is 1, 2, 3, or 4; and x is 0, 1, or 2.

In some embodiments, the compound of Formula (III) is a compound of Formula (III-a):

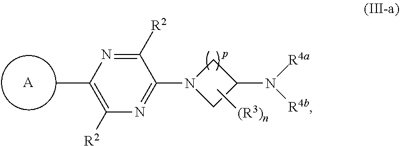

or a pharmaceutically acceptable salt, solvate, hydrate, tautomer, or stereoisomer thereof, wherein A is bicyclic heteroaryl optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R6 are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; and x is 0, 1, or 2.

In some embodiments, the compound of Formula (III) is a compound of Formula (III-a):

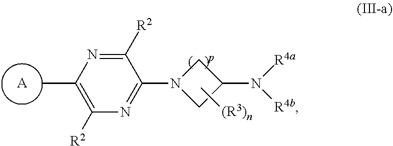

or a pharmaceutically acceptable salt, solvate, hydrate, tautomer, or stereoisomer thereof, wherein A is bicyclic heteroaryl optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R6 are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; and x is 0, 1, or 2.

In some embodiments, the compound of Formula (III) is a compound of Formula (III-b):

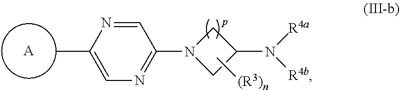

or a pharmaceutically acceptable salt, solvate, hydrate, tautomer, or stereoisomer thereof, wherein A is bicyclic heteroaryl optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NBC(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; and x is 0, 1, or 2.

In some embodiments, the compound of Formula (III) is a compound of Formula (III-c):

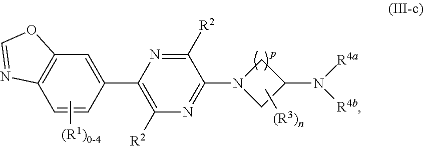

or a pharmaceutically acceptable salt, solvate, hydrate, tautomer, or stereoisomer thereof, wherein each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NBC(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen, C₁-C₆-alkyl, C₂-C₆-alkenyl, C₂-C₆-alkynyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; and x is 0, 1, or 2.

In some embodiments, the compound of Formula (III) is a compound of Formula (III-d):

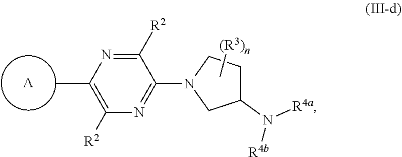

or a pharmaceutically acceptable salt, solvate, hydrate, tautomer, or stereoisomer thereof, wherein A is bicyclic heteroaryl optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen, C₁-C₆-alkyl, C₂-C₆-alkenyl, C₂-C₆-alkynyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; and x is 0, 1, or 2.

In some embodiments, the compound of Formula (III) is a compound of Formula (III-e):

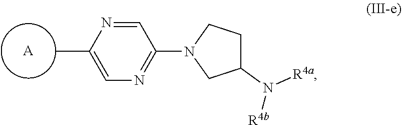

or a pharmaceutically acceptable salt, solvate, hydrate, tautomer, or stereoisomer thereof, wherein A is bicyclic heteroaryl optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —R^{B}C(O)R^{D}—NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; and x is 0, 1, or 2.

In some embodiments, A is selected from

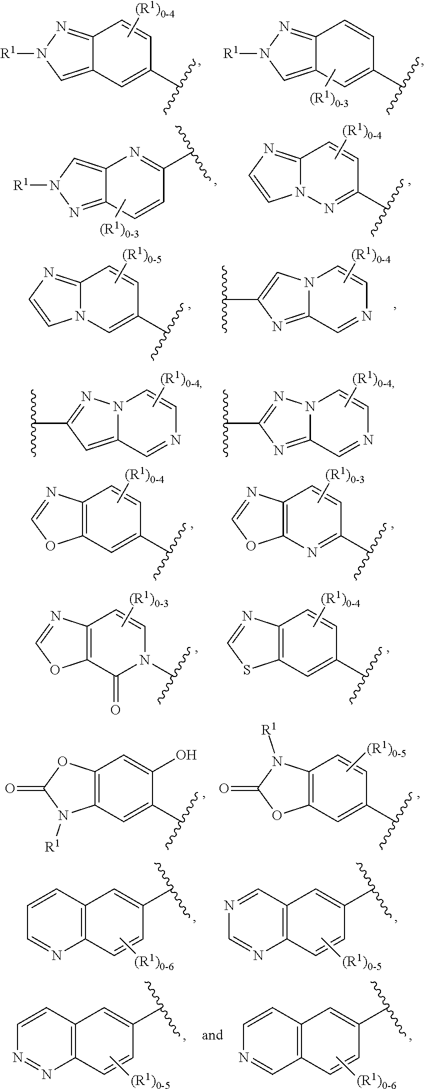

wherein R¹ is as described herein. In some embodiments, A is selected from

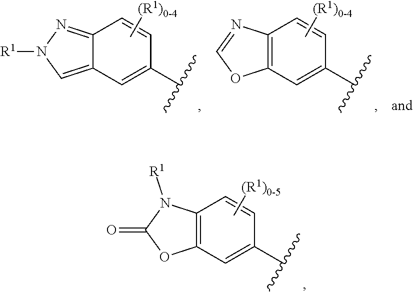

wherein R¹ is as defined herein. In some embodiments, A is selected from

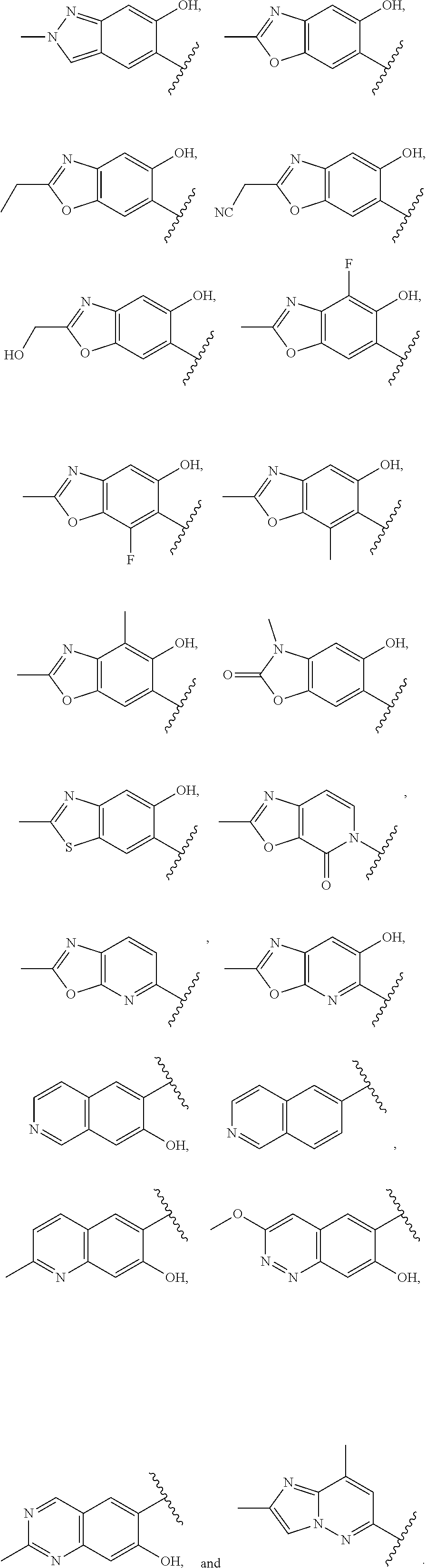

In some embodiments, A is selected from

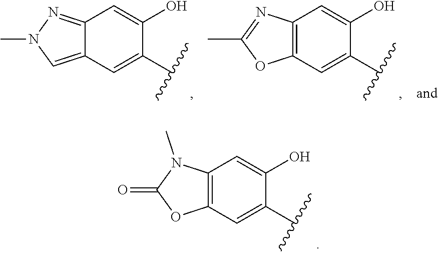 [SMILES: *c1cc2cn(C)nc2cc1O ; *c1cc2oc(C)nc2cc1O ; *c1cc2oc(=O)n(C)c2cc1O]

In some embodiments, the compound of Formula (IV) is a compound of Formula (IV-a):

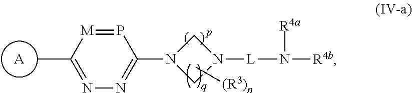

or a pharmaceutically acceptable salt, solvate, hydrate, tautomer, or stereoisomer thereof, wherein A is heteroaryl optionally substituted with one or more R¹; M and P are each independently C(R²) or N; L is absent, C₁-C₆-alkylene, C₂-C₆-alkenylene, C₁-C₆-heteroalkylene, —C(O)—, —NR^{B}C(O)—, —C(O)NR^{B}—, or each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A}; each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NBC(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷; each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶; R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁸ is C₁-C₆-alkyl, halo, or cycloalkyl; n is 0, 1, 2, 3, 4, 5, 6, 7, 8, 9, 10, or 11; p is 1, 2, 3, or 4; q is 0, 1, 2, or 3; and x is 0, 1, or 2.

In some embodiments, the compound of Formula (II), (III), or (IV) is selected from a compound in Table 2, or a pharmaceutically acceptable salt thereof.

| Compound No. | Structure |
|---|---|
| 800 | 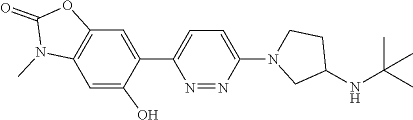 [SMILES: Cn1c(=O)oc2cc(-c3ccc(N4CCC(NC(C)(C)C)C4)nn3)c(O)cc21] |
| 801 | 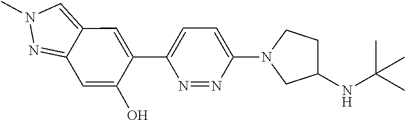 [SMILES: Cn1cc2cc(-c3ccc(N4CCC(NC(C)(C)C)C4)nn3)c(O)cc2n1] |
| 802 | 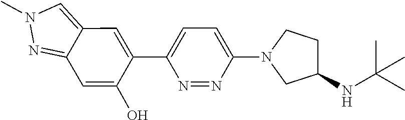 [SMILES: Cn1cc2cc(-c3ccc(N4CC[C@@H](NC(C)(C)C)C4)nn3)c(O)cc2n1] |
| 803 | 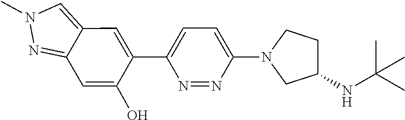 [SMILES: Cn1cc2cc(-c3ccc(N4CC[C@H](NC(C)(C)C)C4)nn3)c(O)cc2n1] |
| 804 | 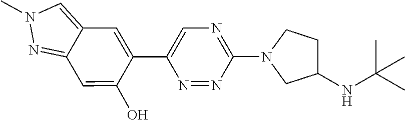 [SMILES: Cn1cc2cc(-c3cnc(N4CCC(NC(C)(C)C)C4)nn3)c(O)cc2n1] |
| 805 | 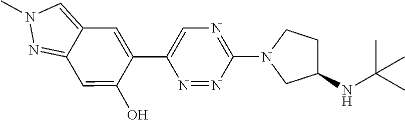 [SMILES: Cn1cc2cc(-c3cnc(N4CC[C@@H](NC(C)(C)C)C4)nn3)c(O)cc2n1] |
| 806 | 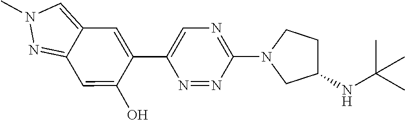 [SMILES: Cn1cc2cc(-c3cnc(N4CC[C@H](NC(C)(C)C)C4)nn3)c(O)cc2n1] |
| 807 | 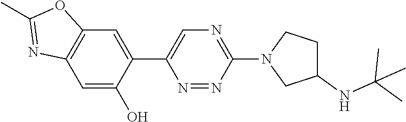 [SMILES: Cc1nc2cc(O)c(-c3cnc(N4CCC(NC(C)(C)C)C4)nn3)cc2o1] |
| 808 | 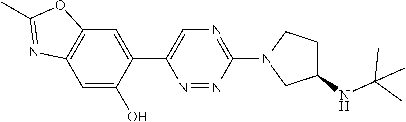 [SMILES: Cc1nc2cc(O)c(-c3cnc(N4CC[C@@H](NC(C)(C)C)C4)nn3)cc2o1] |
| 809 | 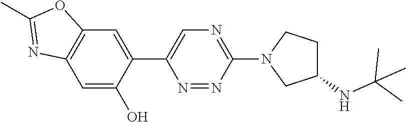 [SMILES: Cc1nc2cc(O)c(-c3cnc(N4CC[C@H](NC(C)(C)C)C4)nn3)cc2o1] |
| 810 | 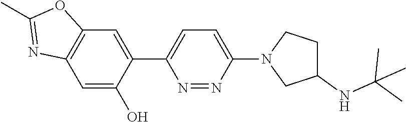 [SMILES: Cc1nc2cc(O)c(-c3ccc(N4CCC(NC(C)(C)C)C4)nn3)cc2o1] |
| 811 | 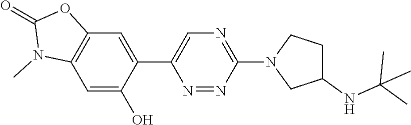 [SMILES: Cn1c(=O)oc2cc(-c3cnc(N4CCC(NC(C)(C)C)C4)nn3)c(O)cc21] |
| 812 | 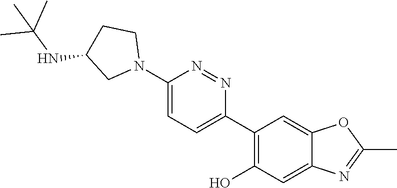 [SMILES: Cc1nc2cc(O)c(-c3ccc(N4CC[C@@H](NC(C)(C)C)C4)nn3)cc2o1] |
| 813 | 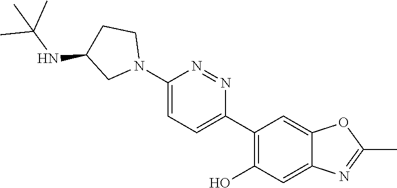 [SMILES: Cc1nc2cc(O)c(-c3ccc(N4CC[C@H](NC(C)(C)C)C4)nn3)cc2o1] |
| 814 | 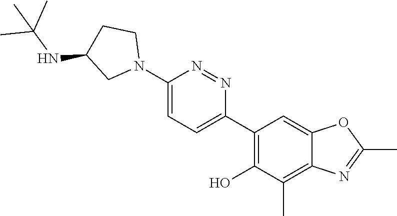 [SMILES: Cc1nc2c(C)c(O)c(-c3ccc(N4CC[C@H](NC(C)(C)C)C4)nn3)cc2o1] |
| 815 | 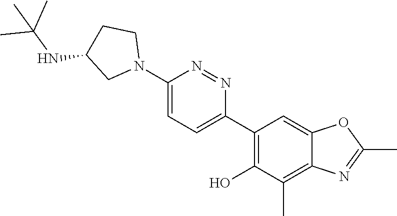 [SMILES: Cc1nc2c(C)c(O)c(-c3ccc(N4CC[C@@H](NC(C)(C)C)C4)nn3)cc2o1] |
| 816 | 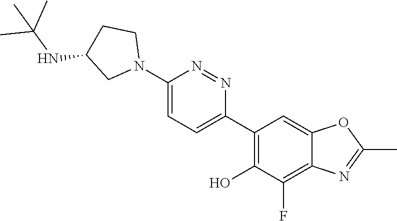 [SMILES: Cc1nc2c(F)c(O)c(-c3ccc(N4CC[C@@H](NC(C)(C)C)C4)nn3)cc2o1] |
| 817 | 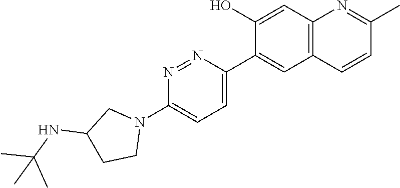 [SMILES: Cc1ccc2cc(-c3ccc(N4CCC(NC(C)(C)C)C4)nn3)c(O)cc2n1] |
| 818 | 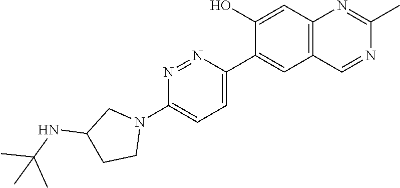 [SMILES: Cc1ncc2cc(-c3ccc(N4CCC(NC(C)(C)C)C4)nn3)c(O)cc2n1] |
| 819 | 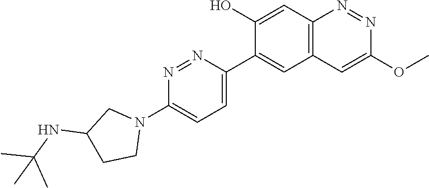 [SMILES: COc1cc2cc(-c3ccc(N4CCC(NC(C)(C)C)C4)nn3)c(O)cc2nn1] |
| 820 | 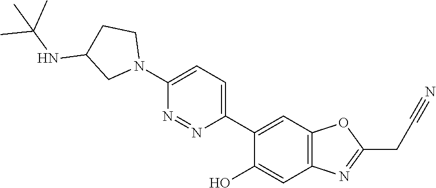 [SMILES: CC(C)(C)NC1CCN(c2ccc(-c3cc4oc(CC#N)nc4cc3O)nn2)C1] |
| 821 | 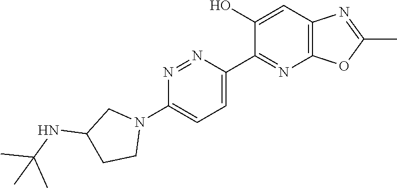 [SMILES: Cc1nc2cc(O)c(-c3ccc(N4CCC(NC(C)(C)C)C4)nn3)nc2o1] |
| 822 | 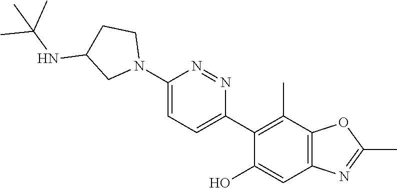 [SMILES: Cc1nc2cc(O)c(-c3ccc(N4CCC(NC(C)(C)C)C4)nn3)c(C)c2o1] |
| 823 | 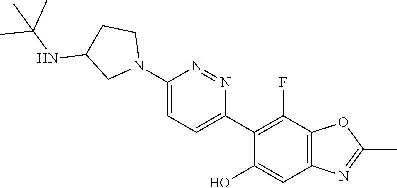 [SMILES: Cc1nc2cc(O)c(-c3ccc(N4CCC(NC(C)(C)C)C4)nn3)c(F)c2o1] |
| 824 | 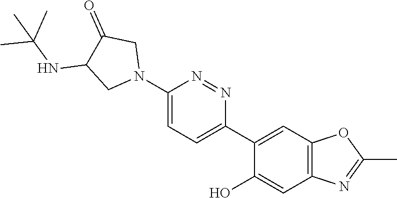 [SMILES: Cc1nc2cc(O)c(-c3ccc(N4CC(=O)C(NC(C)(C)C)C4)nn3)cc2o1] |
| 825 | 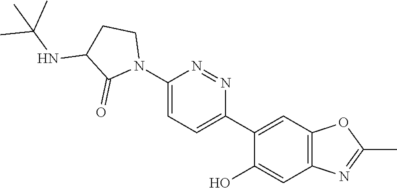 [SMILES: Cc1nc2cc(O)c(-c3ccc(N4CCC(NC(C)(C)C)C4=O)nn3)cc2o1] |
| 826 | 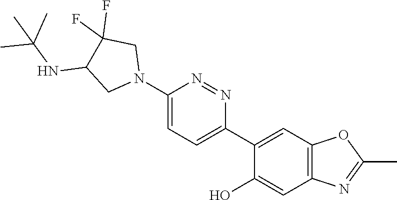 [SMILES: Cc1nc2cc(O)c(-c3ccc(N4CC(NC(C)(C)C)C(F)(F)C4)nn3)cc2o1] |
| 827 | 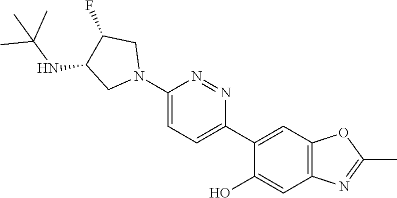 [SMILES: Cc1nc2cc(O)c(-c3ccc(N4C[C@@H](F)[C@@H](NC(C)(C)C)C4)nn3)cc2o1] |
| 828 | 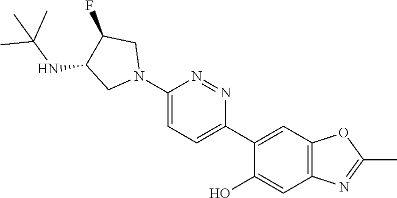 [SMILES: Cc1nc2cc(O)c(-c3ccc(N4C[C@H](NC(C)(C)C)[C@@H](F)C4)nn3)cc2o1] |
| 829 | 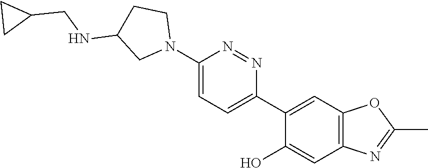 [SMILES: Cc1nc2cc(O)c(-c3ccc(N4CCC(NCC5CC5)C4)nn3)cc2o1] |
| 830 | 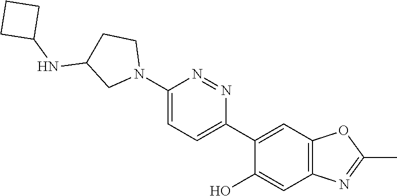 [SMILES: Cc1nc2cc(O)c(-c3ccc(N4CCC(NC5CCC5)C4)nn3)cc2o1] |
| 831 | 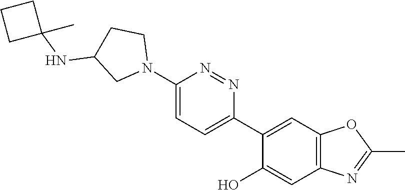 [SMILES: Cc1nc2cc(O)c(-c3ccc(N4CCC(NC5(C)CCC5)C4)nn3)cc2o1] |
| 832 | 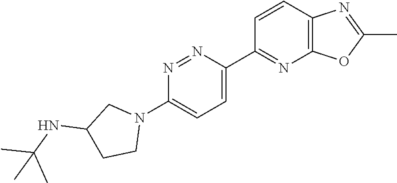 [SMILES: Cc1nc2ccc(-c3ccc(N4CCC(NC(C)(C)C)C4)nn3)nc2o1] |
| 833 | 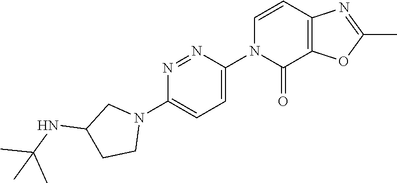 [SMILES: Cc1nc2ccn(-c3ccc(N4CCC(NC(C)(C)C)C4)nn3)c(=O)c2o1] |
| 834 | 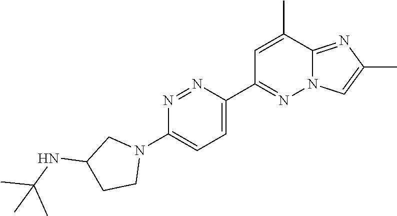 [SMILES: Cc1cn2nc(-c3ccc(N4CCC(NC(C)(C)C)C4)nn3)cc(C)c2n1] |
| 835 | 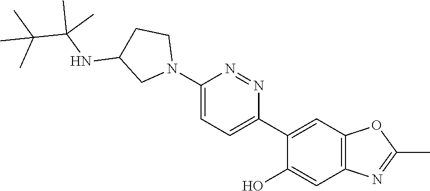 [SMILES: Cc1nc2cc(O)c(-c3ccc(N4CCC(NC(C)(C)C(C)(C)C)C4)nn3)cc2o1] |
| 836 | 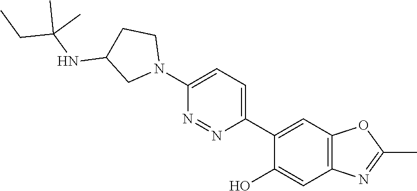 [SMILES: CCC(C)(C)NC1CCN(c2ccc(-c3cc4oc(C)nc4cc3O)nn2)C1] |
| 837 | 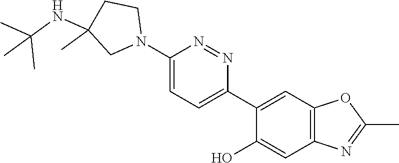 [SMILES: Cc1nc2cc(O)c(-c3ccc(N4CCC(C)(NC(C)(C)C)C4)nn3)cc2o1] |
| 838 | 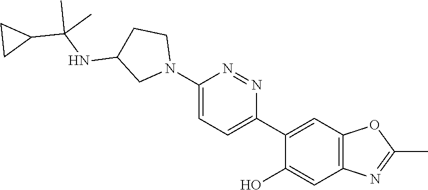 [SMILES: Cc1nc2cc(O)c(-c3ccc(N4CCC(NC(C)(C)C5CC5)C4)nn3)cc2o1] |
| 839 | 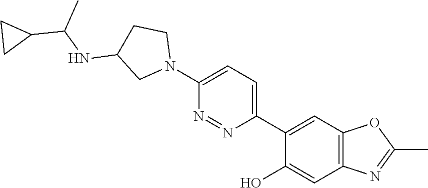 [SMILES: Cc1nc2cc(O)c(-c3ccc(N4CCC(NC(C)C5CC5)C4)nn3)cc2o1] |
| 840 | 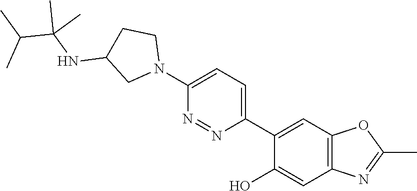 [SMILES: Cc1nc2cc(O)c(-c3ccc(N4CCC(NC(C)(C)C(C)C)C4)nn3)cc2o1] |
| 841 | 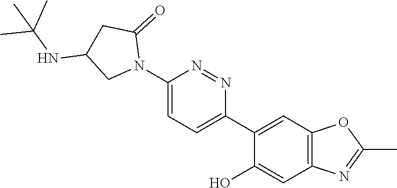 [SMILES: Cc1nc2cc(O)c(-c3ccc(N4CC(NC(C)(C)C)CC4=O)nn3)cc2o1] |
| 842 | 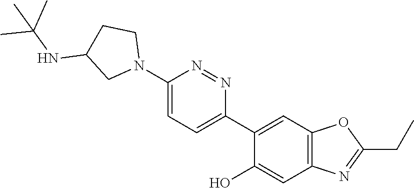 [SMILES: CCc1nc2cc(O)c(-c3ccc(N4CCC(NC(C)(C)C)C4)nn3)cc2o1] |
| 843 | 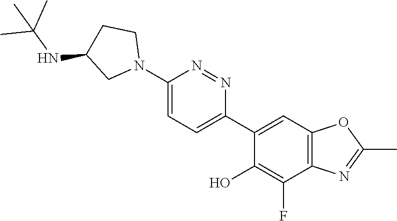 [SMILES: Cc1nc2c(F)c(O)c(-c3ccc(N4CC[C@H](NC(C)(C)C)C4)nn3)cc2o1] |
| 844 | 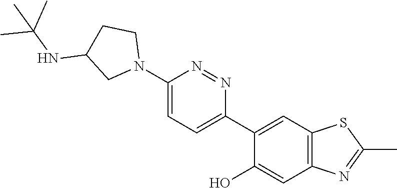 [SMILES: Cc1nc2cc(O)c(-c3ccc(N4CCC(NC(C)(C)C)C4)nn3)cc2s1] |
| 845 | 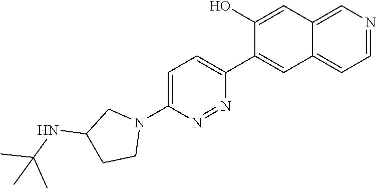 [SMILES: CC(C)(C)NC1CCN(c2ccc(-c3cc4ccncc4cc3O)nn2)C1] |
| 846 | 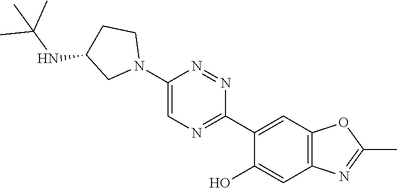 [SMILES: Cc1nc2cc(O)c(-c3ncc(N4CC[C@@H](NC(C)(C)C)C4)nn3)cc2o1] |
| 847 | 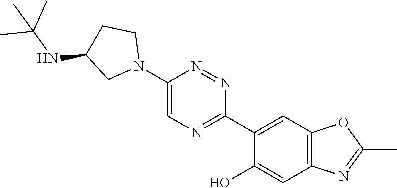 [SMILES: Cc1nc2cc(O)c(-c3ncc(N4CC[C@H](NC(C)(C)C)C4)nn3)cc2o1] |
| 848 | 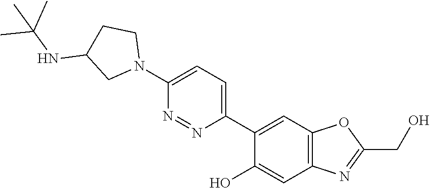 [SMILES: CC(C)(C)NC1CCN(c2ccc(-c3cc4oc(CO)nc4cc3O)nn2)C1] |
| 849 | 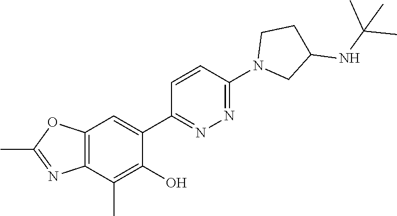 [SMILES: Cc1nc2c(C)c(O)c(-c3ccc(N4CCC(NC(C)(C)C)C4)nn3)cc2o1] |
| 850 | 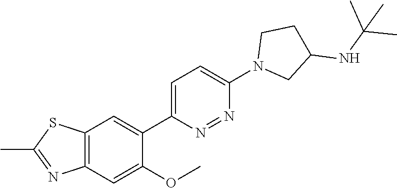 [SMILES: COc1cc2nc(C)sc2cc1-c1ccc(N2CCC(NC(C)(C)C)C2)nn1] |
| 851 | 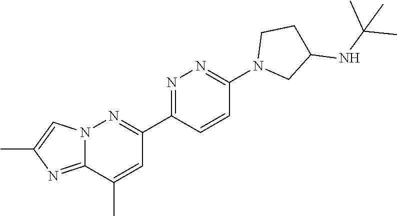 [SMILES: Cc1cn2nc(-c3ccc(N4CCC(NC(C)(C)C)C4)nn3)cc(C)c2n1] |
| 852 | 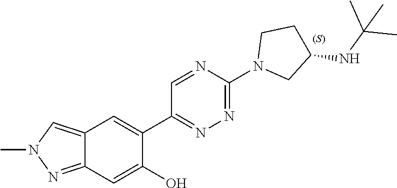 |
| 853 | 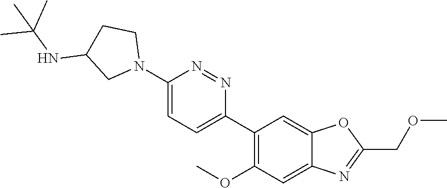 [SMILES: COCc1nc2cc(OC)c(-c3ccc(N4CCC(NC(C)(C)C)C4)nn3)cc2o1] |
| 854 | 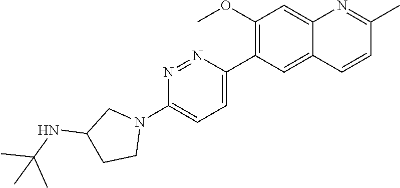 [SMILES: COc1cc2nc(C)ccc2cc1-c1ccc(N2CCC(NC(C)(C)C)C2)nn1] |
| 855 | 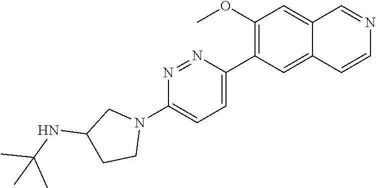 [SMILES: COc1cc2cnccc2cc1-c1ccc(N2CCC(NC(C)(C)C)C2)nn1] |
| 856 | 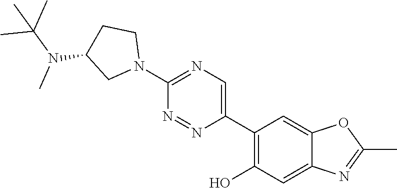 [SMILES: Cc1nc2cc(O)c(-c3cnc(N4CC[C@@H](N(C)C(C)(C)C)C4)nn3)cc2o1] |
| 857 | 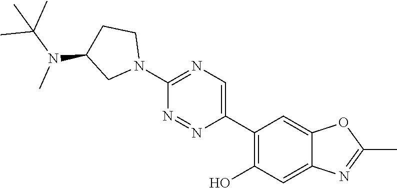 [SMILES: Cc1nc2cc(O)c(-c3cnc(N4CC[C@H](N(C)C(C)(C)C)C4)nn3)cc2o1] |
| 858 | 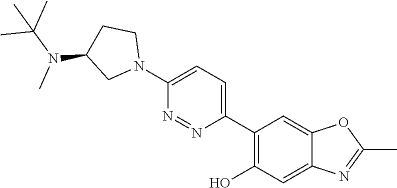 [SMILES: Cc1nc2cc(O)c(-c3ccc(N4CC[C@H](N(C)C(C)(C)C)C4)nn3)cc2o1] |
| 859 | 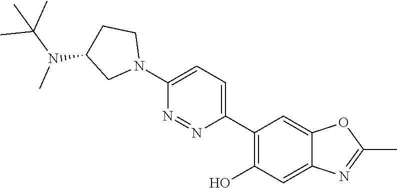 [SMILES: Cc1nc2cc(O)c(-c3ccc(N4CC[C@@H](N(C)C(C)(C)C)C4)nn3)cc2o1] |
| 860 | 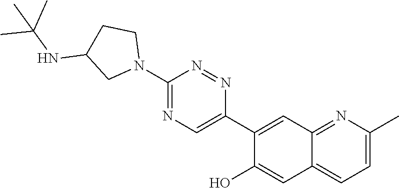 |
| 861 | 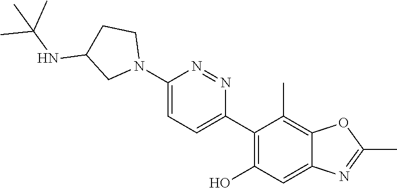 [SMILES: Cc1nc2cc(O)c(-c3ccc(N4CCC(NC(C)(C)C)C4)nn3)c(C)c2o1] |
| 862 | 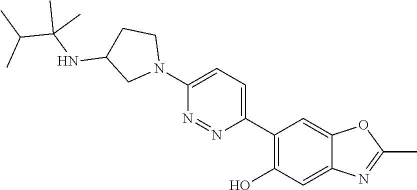 [SMILES: Cc1nc2cc(O)c(-c3ccc(N4CCC(NC(C)(C)C(C)C)C4)nn3)cc2o1] |
| 863 | 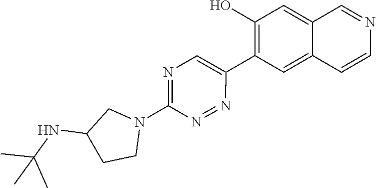 |
| 864 | 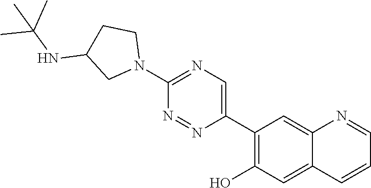 |
| 865 | 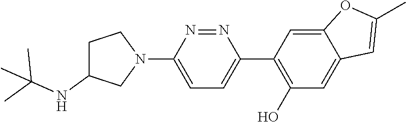 [SMILES: Cc1cc2cc(O)c(-c3ccc(N4CCC(NC(C)(C)C)C4)nn3)cc2o1] |
| 866 | 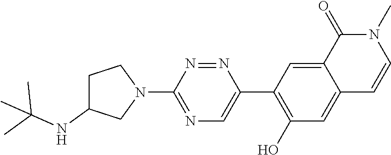 [SMILES: Cn1ccc2cc(O)c(-c3cnc(N4CCC(NC(C)(C)C)C4)nn3)cc2c1=O] |
| 867 | 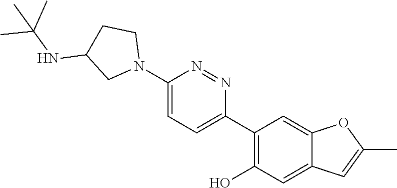 [SMILES: Cc1cc2cc(O)c(-c3ccc(N4CCC(NC(C)(C)C)C4)nn3)cc2o1] |
| 868 | 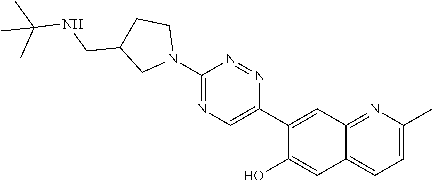 [SMILES: Cc1ccc2cc(O)c(-c3cnc(N4CCC(CNC(C)(C)C)C4)nn3)cc2n1] |
| 869 | 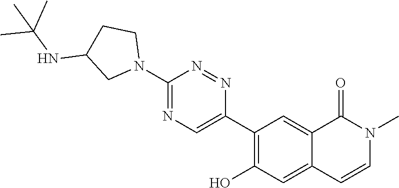 [SMILES: Cn1ccc2cc(O)c(-c3cnc(N4CCC(NC(C)(C)C)C4)nn3)cc2c1=O] |
| 870 | 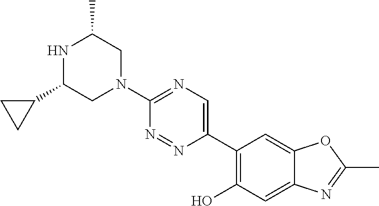 [SMILES: Cc1nc2cc(O)c(-c3cnc(N4C[C@@H](C)N[C@@H](C5CC5)C4)nn3)cc2o1] |
| 871 | 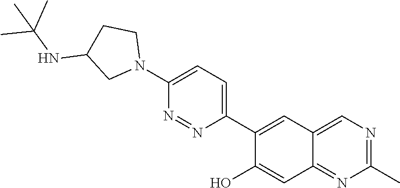 [SMILES: Cc1ncc2cc(-c3ccc(N4CCC(NC(C)(C)C)C4)nn3)c(O)cc2n1] |
| 872 | 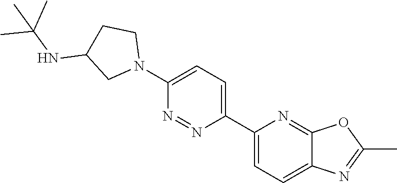 [SMILES: Cc1nc2ccc(-c3ccc(N4CCC(NC(C)(C)C)C4)nn3)nc2o1] |
| 873 | 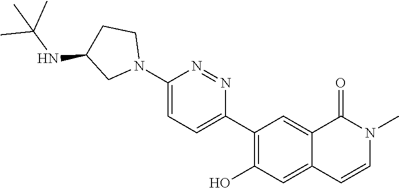 [SMILES: Cn1ccc2cc(O)c(-c3ccc(N4CC[C@H](NC(C)(C)C)C4)nn3)cc2c1=O] |
| 874 | 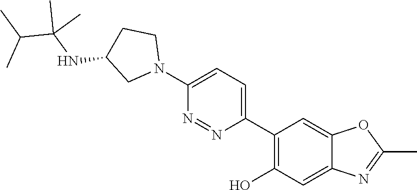 [SMILES: Cc1nc2cc(O)c(-c3ccc(N4CC[C@@H](NC(C)(C)C(C)C)C4)nn3)cc2o1] |
| 875 | 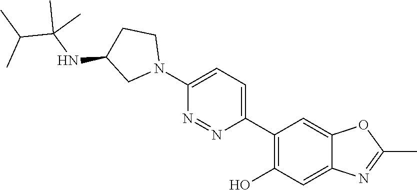 [SMILES: Cc1nc2cc(O)c(-c3ccc(N4CC[C@H](NC(C)(C)C(C)C)C4)nn3)cc2o1] |
| 876 | 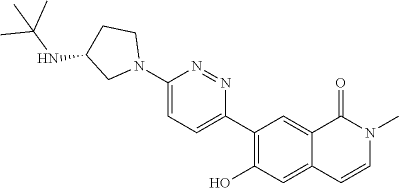 [SMILES: Cn1ccc2cc(O)c(-c3ccc(N4CC[C@@H](NC(C)(C)C)C4)nn3)cc2c1=O] |
| 877 | 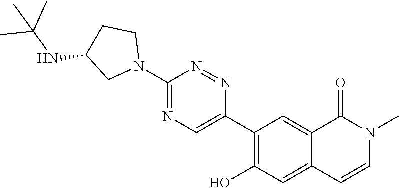 [SMILES: Cn1ccc2cc(O)c(-c3cnc(N4CC[C@@H](NC(C)(C)C)C4)nn3)cc2c1=O] |
| 878 | 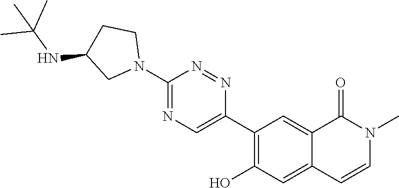 [SMILES: Cn1ccc2cc(O)c(-c3cnc(N4CC[C@H](NC(C)(C)C)C4)nn3)cc2c1=O] |

### Pharmaceutical Compositions, Kits, and Administration

The present invention provides pharmaceutical compositions comprising a compound of Formula (I), (II), (III), (IV) e.g., a compound of Formula (I), (II), (III), (IV) or a pharmaceutically acceptable salt, solvate, hydrate, tautomer, or stereoisomer, as described herein, and optionally a pharmaceutically acceptable excipient. In certain embodiments, the pharmaceutical composition described herein comprises a compound of Formula (I), (II), (III), (IV) or a pharmaceutically acceptable salt thereof, and optionally a pharmaceutically acceptable excipient. In certain embodiments, the compound of Formula (I), (II), (III), (IV) or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I), (II), (III), (IV) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension.

These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I), (II), (III), (IV) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a compound described herein, or a pharmaceutically acceptable salt, solvate, hydrate, tautomer, or stereoisomer thereof, or a pharmaceutical composition thereof. In certain embodiments, the kit of the disclosure includes a first container comprising a compound described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the compound, or a pharmaceutically acceptable salt, solvate, hydrate, tautomer, or stereoisomer thereof, or a pharmaceutical composition thereof, to a subject to prevent and/or treat a proliferative disease or a non-proliferative disease.

### Methods of Use

Described herein are compounds useful for modulating splicing. In some embodiments, a compound of Formula (I), (II), (III), (IV) may be used to alter the amount, structure, or composition of a nucleic acid (e.g., a precursor RNA, e.g., a pre-mRNA, or the resulting mRNA) by increasing or decreasing splicing at a splice site. In some embodiments, increasing or decreasing splicing results in modulating the level or structure of a gene product (e.g., an RNA or protein) produced. In some embodiments, a compound of Formula (I), (II), (III), (IV) may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a precursor RNA, e.g., a pre-mRNA) through inclusion of a splice site in the target, wherein the method comprises providing a compound of Formula (I), (II), (III), (IV). In some embodiments, inclusion of a splice site in a target (e.g., a precursor RNA, e.g., a pre-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a precursor RNA, e.g., a pre-mRNA, or the resulting mRNA) through exclusion of a splice site in the target, wherein the method comprises providing a compound of Formula (I), (II), (III), (IV).

In some embodiments, exclusion of a splice site in a target (e.g., a precursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a precursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35%, 40%, 45%, 50%, 55%, 60%, 65%, 70%, 75%, 80%, 85%, 90%, 95%, 99%, or more), e.g., as compared to a reference (e.g., the absence of a compound of Formula (I), (II), (III), (IV), or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, ABCA4, ABCA9, ABCB1, ABCB5, ABCC9, ABCD1, ACADL, ACADM, ACADSB, ACSS2, ACTB, ACTG2, ADA, ADAL, ADAM10, ADAM15, ADAM22, ADAM32, ADAMTS12, ADAMTS13, ADAMTS20, ADAMTS6, ADAMTS9, ADAR, ADCY3, ADCY10, ADCY8, ADNP, ADRBK2, AFP, AGL, AGT, AHCTF1, AHR, AKAP10, AKAP3, AKNA, ALAS1, ALS2CL, ALB, ALDH3A2, ALG6, AMBRA1, ANK3, ANTXR2, ANXA10, ANXA11, ANGPTL3, AP2A2, AP4E1, APC, APOA1, APOB, APOC3, APOH, AR, ARID2, ARID3A, ARID3B, ARFGEF1, ARFGEF2, ARHGAP1, ARHGAP8, ARHGAP18, ARHGAP26, ARHGEF18, ARHGEF2, ARPC3, ARS2, ASH1L, ASHIL-IT1, ASNSD1, ASPM, ATAD5, ATF1, ATG4A, ATG16L2, ATM, ATN1, ATP11C, ATP6V1G3, ATP13A5, ATP7A, ATP7B, ATR, ATXN2, ATXN3, ATXN7, ATXN10, AXIN1, B2M, B4GALNT3, BBS4, BCL2, BCL2L1, BCL2-like 11 (BIM), BCL11B, BBOX1, BCS1L, BEAN1, BHLHE40, BMPR2, BMP2K, BPTF, BRAF, BRCA1, BRCA2, BRCC3, BRSK1, BRSK2, BTAF1, BTK, C2orf55, C4orf29, C6orf118, C9orf43, C9orf72, C10orf137, C11orf3O, C11orf65, C11orf70, C11orf87, C12orf51, C13orf1, C13orf15, C14orf01, C14orf118, C15orf29, C15orf42, C15orf60, C16orf33, C16orf38, C16orf48, C18orf8, C19orf42, C1orf107, C1orf114, C1orf130, C1orf149, C1orf27, C1orf71, C1orf94, C1R, C20orf74, C21orf70, C3orf23, C4orf18, C5orf34, C8B, C8orf33, C9orf114, C9orf86, C9orf98, C3, CA11, CAB39, CACHD1, CACNA1A, CACNA1B, CACNA1C, CACNA2D1, CACNA1G, CACNA1H, CALCA, CALCOCO2, CAMK1D, CAMKK1, CAPN3, CAPN9, CAPSL, CARD11, CARKD, CASZ1, CAT, CBLB, CBX1, CBX3, CCDC102B, CCDC11, CCDC15, CCDC18, CCDC5, CCDC81, CCDC131, CCDC146, CD4, CD274, CD1B, CDC14A, CDC16, CDC2L5, CDC42BPB, CDCA8, CDH10, CDH11, CDH24, CDH8, CDH9, CDK5RAP2, CDK6, CDK8, CDK11B, CD33, CD46, CDH1, CDH23, CDK6, CDK11B, CDK13, CEBPZ, CEL, CELSR3, CENPA, CENPI, CENPT, CENTB2, CENTG2, CEP110, CEP170, CEP192, CETP, CFB, CFTR, CFH, CGN, CGNL1, CHAF1A, CHD9, CHIC2, CHL1, CHN1, CHM, CLEC16A, CL1C2, CLCN1, CLINT1, CLK1, CLPB, CLPTM1, CMIP, CMYA5, CNGA3, CNOT1, CNOT7, CNTN6, COG3, COL11A1, COL11A2, COL12A1, COL14A1, COL15A1, COL17AM, COL19A1, COL1A1, COL1A2, COL2A1, COL3A1, COL4A, COL4A2, COL4A5, COL4A6, COL5A2, COL6A1, COL7A, COL9A, COL9A2, COL22A1, COL24A1, COL25A1, COL29A1, COLQ, COMTD1, COPA, COPB2, COPS7B, COPZ2, CPSF2, CPXM2, CR1, CRBN, CRYZ, CREBBP, CRKRS, CSE1L, CSTB, CSTF3, CT45-6, CTNNB1, CUBN, CUL4B, CUL5, CXorf41, CXXC1, CYBB, CYFIP2, CYP3A4, CYP3A43, CYP3A5, CYP4F2, CYP4F3, CYP17, CYP19, CYP24A1, CYP27A1, DAB1, DAZ2, DCBLD1, DCC, DCTN3, DCUN1D4, DDA1, DDEF1, DDX1, DDX24, DDX4, DENND2D, DEPDC2, DES, DGAT2, DHFR, DHRS7, DHRS9, DHX8, DIP2A, DMD, DMTF, DNAH3, DNAH8, DNAI1, DNAJA4, DNAJC13, DNAJC7, DNMT1, DNTTIP2, DOCK4, DOCK5, DOCK10, DOCK11, DOT1L, DPP3, DPP4, DPY19L2P2, DR1, DSCC1, DVL3, DUX4, DYNC1H1, DYSF, E2F1, E2F3, E2F8, E4F1, EBF1, EBF3, ECM2, EDEM3, EFCAB3, EFCAB4B, EFNA4, EFTUD2, EGFR, EIF3A, ELA1, ELA2A, ELF2, ELF3, ELF4, EMCN, EMD, EML5, ENO3, ENPP3, EP300, EPAS1, EPB41L5, EPHA3, EPHA4, EPHB1, EPHB2, EPHB3, EPS15, ERBB4, ERCC1, ERCC8, ERGIC3, ERMN, ERMP1, ERN1, ERN2, ESR1, ESRRG, ETS2, ETV3, ETV4, ETV5, ETV6, EVC2, EWSR1, EXO1, EXOC4, F3, F11, F13A1, F5, F7, F8, FAH, FAM13A1, FAM13B1, FAM13C1, FAM134A, FAM161A, FAM176B, FAM184A, FAM19A1, FAM20A, FAM23B, FAM65C, FANCA, FANCC, FANCG, FANCM, FANK1, FAR2, FBN1, FBXO15, FBX018, FBXO38, FCGBP, FECH, FEZ2, FGA, FGD6, FGFR2, FGFR1OP, FGFR1OP2, FGFR2, FGG, FGR, FIX, FKBP3, FLI1, FLJ35848, FLJ36070, FLNA, FN1, FNBP1L, FOLH1, FOSL1, FOSL2, FOXK1, FOAM1, FOXO1, FOXP4, FRAS1, FUT9, FXN, FZD3, FZD6, GAB1, GABPA, GALC, GALNT3, GAPDH, GART, GAS2L3, GATA3, GATAD2A, GBA, GBGT1, GCG, GCGR, GCK, GFI1, GFM1, GH1, GHR, GHV, GJA1, GLA, GLT8D1, GNA11, GNAQ, GNAS, GNB5, GOLGB1, GOLT1A, GOLT1B, GPATCH1, GPR158, GPR160, GPX4, GRAMD3, GRHL1, GRHL2, GRHPR, GRIA1, GRIA3, GRIA4, GRIN2B, GRM3, GRM4, GRN, GSDMB, GSTCD, GSTO2, GTF2I, GTPBP4, HADHA, HAND2, HBA2, HBB, HCK, HDAC3, HDAC5, HDX, HEPACAM2, HERC1, HES7, HEXA, HEXB, HHEX, HIPK3, HLA-DPB1, HLA-G, HLCS, HLTF, HMBS, HMGA1, HMGCL, HNF1A, HNF1B, HNF4A, HNF4G, HNRNPH1, HOXC10, HP1BP3, HPGD, HPRT1, HPRT2, HSF1, HSF4, HSF2BP, HSPA9, HSPG2, HTT, HXA, ICA1, IDH1, IDS, IFI44L, IKBKAP, IKZF, IKZF3, IL1R2, IL5RA, IL7RA, IMMT, INPP5D, INSR, INTS3, INTU, IP04, IP08, IQGAP2, IRF2, IRF4, IRF8, IRX3, ISL1, ISL2, ITFG1, ITGA6, ITGAL, ITGB1, ITGB2, 1TGB3, ITGB4, ITIH1, ITPR2, IWS1, JAK1, JAK2, JAG1, JMJD1C, JPH3, KALRN, KAT6A, KATNAL2, KCNN2, KCNT2, KDM2A, KIAA0256, KIAA0528, KIAA0564, KIAA0586, KIAA1033, KIAA1166, KIAA1219, KIAA1409, KIAA1622, KIAA1787, KIF3B, KIF15, KIF16B, KIFSA, KIF5B, KIF9, KIN, KIR2DL5B, KIR3DL2, KIR3DL3, KIT, KLF3, KLF5, KLF7, KLFO, KLF12, KLF16, KLHL20, KLK12, KLKBI, KMT2A, KMT2B, KPNAS, KRAS, KREMEN1, KRIT1, KRT5, KRTCAP2, KYNU, LICAM, L3MBTL, L3MBTL2, LACE1, LAMA1, LAMA2, LAMA3, LAMB1, LARP7, LDLR, LEF1, LENG1, LGALS3, LGMN, LHCGR, LHX3, LHX6, LIMCHI, LIMK2, LIN28B, LIN54, LMBRD1, LMBRD2, LMLN, LMNA, LMO2, LMO7, LOC389634, LOC390110, LPA, LPCAT2, LPL, LRP4, LRPPR^{C}, LRRK2, LRRC19, LRRC42, LRWD1, LUM, LVRN, LYN, LYST, MADD, MAGI1, MAGT1, MALT1, MAP2K1, MAP4K4, MAPK8IP3, MAPK9, MAPT, MARC1, MARCHS, MATN2, MBD3, MCF2L2, MCM6, MDGA2, MDM4, ASXL1, FUS, SPR54, MECOM, MEF2C, MEF2D, MEGF0, MEGF1, MEMO1, METL MGA, MGAM, MGAT4A, MGAT5, MGC16169, MGC34774, MKKS, MIB1, MIER2, MITF, MKL2, MLANA, MLH1, MLLS, MLX, MME, MPDZ, MPI, MRAP2, MRPL11, MRPL39, MRPS28, MRPS35, MS4A13, MSH2, MSH3, MSMB, MST1R, MTDH, MTERF3, MTF1, MTF2, MTIF2, MTHFR, MUC2, MUT, MVK, MYB, MYBL2, MYC, MYCBP2, MYH2, MYRF, MYT1, MY019, MY03A, MY09B, MYOM2, MYOM3, NAG, NARG1, NARG2, NCOAJ, NDC80, NDFIP2, NEB, NEDD4, NEK, NEKS, NEK1, NF, NF2, NFATC2, NFE2L2, NFIA, NFIB, NFIX, NFKB1, NFKB2, NFKBIL2, NFRKB, NFYA, NFYB, NIPA2, NKAIN2, NKAP, NLRC3, NLRC5, NLRP3, NLRP7, NLRP8, NLRP13, NME1, NME1-NME2, NME2, NME7, NOL10, NOP561, NOS1, NOS2A, NOTCH1, NPAS4, NPM1, NRID1, NRIH3, NRIH4, NR4A3, NR5A1, NRXNI, NSMAF, NSMCE2, NT5C, NT5C2, NT5C3, NUBP1, NUBPL, NUDT5, NUMA1, NUP88, NUP98, NUP160, NUPL1, OAT, OAZ1, OBFC2A, OBFC2B, OLIG2, OMA1, OPAl, OPN4, OPTN, OSBPLI1, OSBPL8, OSGEPLI, OTC, OTX2, OVOL2, OXT, PA2G4, PADI4, PAH, PAN2, PAOX, PAPOLG, PARD3, PARP1, PARVB, PAWR, PAX3, PAX8, PBGD, PBRM1, PBX2, PCBP4, PCCA, PCGF2, PCNX, PCOTH, PDCD4, PDE4D, PDE8B, PDE10A, PD1A3, PDH1, PDLIM5, PDXK, PDZRN3, PELI2, PDK4, PDS5A, PDS5B, PGK1, PGM2, PHACTR4, PHEX, PHKB, PHLDB2, PHOX2B, PHTF1, PIAS1, PIEZO1, PIGF, PIGN, PIGT, PIK3C2G, PIK3CA, PIK3CD, PIK3CG, PIK3RI, PIP5K1A, PITRM1, PIWIL3, PKD1, PKHD1L1, PKD2, PKIB, PKLR, PKM1, PKM2, PLAGL2, PLCB1, PLCB4, PLCG1, PLD1, PLEKHA5, PLEKHA7, PLEKHM1, PLKR, PLXNCI, PMFBP1, POLN, POLR3D, POMT2, POSTN, POU2AF1, POU2F2, POU2F3, PPARA, PPFIA2, PPP1R12A, PPP3CB, PPP4C, PPP4RIL, PPP4R2, PRAME, PRC1, PRDM1, PREX1, PREX2, PRIM1, PRIM2, PRKARIA, PRKCA, PRKG1, PRMT7, PROC, PROCR, PROSC, PRODH, PROX1, PRPF40B, PRPF4B, PRRG2, PRUNE2, PSD3, PSEN1, PSMAL, PTCH1, PTEN, PTK2, PTK2B, PTPN2, PTPN3, PTPN4, PTPN11, PTPN22, PTPRD, PTPRK, PTPRM, PTPRN2, PTPRT, PUSIO, PVRL2, PYGM, QRSLI, RABI1FIP2, RAB23, RAF1, RALBP1, RALGDS, RBICCI, RBL2, RBM39, RBM45, RBPJ, RBSN, REC8, RELB, RFC4, RFTJ, RFTN1, RHOA, RHPN2, RIF1, RIT1, RLN3, RMND5B, RNFI1, RNF32, RNFT1, RNGTT, ROCK1, ROCK2, RORA, RP1, RP6KA3, RP11-265F1, RP13-36C9, RPAP3, RPNI, RPGR, RPL22, RPL22L1, RPS6KA6, RREB1, RRM1, RRP1B, RSK2, RTEL1, RTF1, RUFY1, RUNX1, RUNX2, RXRA, RYR3, SAAL1, SAE1, SALL4, SAT1, SATB2, SBCAD, SCNIA, SCN2A, SCN3A, SCN4A, SCN5A, SCN8A, SCNA, SCNIJA, SCO1, SCYL3, SDCJ, SDK1, SDK2, SEC24A, SEC24D, SEC31A, SELIL, SENP3, SENP6, SENP7, SERPINA1, SETD3, SETD4, SETDBI, SEZ6, SFRS12, SGCE, SGOL2, SGPLI, SH2DIA, SH3BGRL2, SH3PXD2A, SH3PXD2B, SH3RF2, SH3TC2, SHOC2, SIPAIL2, SIPAIL3, SIVA1, SKAP1, SKIV2L2, SLC6A11, SLC6A13, SLC6A6, SLC7A2, SLC12A3, SLC13A1, SLC22A17, SLC25A14, SLC28A3, SLC33A1, SLC35F6, SLC38A1, SLC38A4, SLC39A10, SLC4A2, SLC6A8, SMARCA1, SMARCA2, SMARCAS, SMARCC2, SMC5, SMN2, SMOX, SMS, SMTN, SNCAIP, SNORD86, SNRK, SNRP70, SNX5, SNX6, SOD1, SOD10, SOS, SOS2, SOX5, SOX6, SOX8, SP1, SP2, SP3, SPH1O, SPAG9, SPATA13, SPATA4, SPATS1, SPECCIL, SPDEF, SPI1, SPINKS, SPP2, SPTAI, SRF, SRM, SRP72, SSX3, SSX5, SSX9, STAG1, STAG2, STAMBPLI, STARD6, STAT1, STAT3, STAT5A, STAT5B, STAT6, STK17B, STX3, STXBPJ, SUCLG2, SULF2, SUPT6H, SUPT16H, SV2C, SYCP2, SYT6, SYCPI, SYTL3, SYTL5, TAF2, TARDBP, TBCID3G, TBCID8B, TBCID26, TBCID29, TBCEL, TBK1, TBP, TBPL1, TBR1, TBX, TCEB3, TCF3, TCF4, TCF7L2, TCFL5, TCF12, TCPIIL2, TDRD3, TEAD1, TEAD3, TEAD4, TECTB, TEK, TERF1, TERF2, TET2, TFAP2A, TFAP2B, TFAP2C, TFAP4, TFDP1, TFR^{C}, TG, TGM7, TGSI, THAP7, THAP12, THOC2, TIAL1, TIAM2, TIMM50, TLK2, TM4SF20, TM6SF1, TMEM27, TMEM77, TMEM156, TMEM194A, TMFI, TMPRSS6, TNFRSF10A, TNFRSF10B, TNFRSF8, TNK2, TNKS, TNKS2, TOM1L1, TOM1L2, TOP2B, TP53, TP53INP1, TP53BP2, TP53I3, TP63, TRAF3IP3, TRAPPC2, TRIM44, TRIM65, TRIML1, TRIML2, TRPM3, TRPM5, TRPM7, TRPSI, TSC1, TSC2, TSHB, TSPAN7, TTC17, TTF1, TTLL5, TTLL9, TTN, TTPAL, TTR, TUSC3, TXNDC10, UBE3A, UCK1, UGT1A1, UHRF1BP1, UNC45B, UNC5C, USH2A, USF2, USP1, USP6, USP18, USP38, USP39, UTP20, UTP15, UTP18, UTRN, UTX, UTY, UVRAG, UXT, VAPA, VEGFA, VPS29, VPS35, VPS39, VT11A, VTI1B, VWA3B, WDFY2, WDR16, WDR17, WDR26, WDR44, WDR67, WDTC1, WRN, WRNIP1, WT1, WWC3, XBPI, XRNI, XRRN2, XX—FW88277, YAP1, YARS, YBX1, YGM, YYJ, ZBTB18, ZBTB20, ZC3HAV1, ZC3HC1, ZC3H7A, ZDHHC19, ZEB1, ZEB2, ZFPM1, ZFYVE1, ZFX, ZIC2, ZNF37A, ZNF91, ZNF114, ZNF155, ZNF169, ZNF205, ZNF236, ZNF317, ZNF320, ZNF326, ZNF335, ZNF365, ZNF367, ZNF407, ZNF468, ZNF506, ZNF511, ZNF511-PRAP1, ZNF519, ZNF521, ZNF592, ZNF618, ZNF763, and ZWINT.

Additional exemplary genes encoding a target sequence (e.g., a target sequence comprising DNA or RNA, e.g., pre-mRNA) include genes include AICF, A4GALT, AAR2, ABAT, ABCA11P, ZNF721, ABCA5, ABHDIO, ABHD13, ABHD2, ABHD6, ACO000120.3, KRIT1, AC004076.1, ZNF772, AC004076.9, ZNF772, AC004223.3, RAD51D, AC004381.6, AC006486.1, ERF, ACO007390.5, AC007780.1, PRKARIA, ACO007998.2, INO80C, ACO009070.1, CMC2, AC009879.2, AC009879.3, ADHFE1, AC010487.3, ZNF816-ZNF321P, ZNF816, AC010328.3, AC010522.1, ZNF587B, AC010547.4, ZNF19, AC012313.3, ZNF497, AC012651.1, CAPN3, AC013489.1, DET1, AC016747.4, C2orf74, AC020907.6, FXYD3, AC021087.5, PDCD6, AHRR, AC022137.3, ZNF761, AC025283.3, NAA60, AC027644.4, RABGEFJ, AC055811.2, FLCN, AC069368.3, ANKDDIA, AC073610.3, ARF3, AC074091.1, GPNI, AC079447.1, LIPT1, AC092587.1, AC079594.2, TRIM59, AC091060.1, C18orf21, AC092143.3, MCIR, AC093227.2, ZNF607, AC093512.2, ALDOA, AC098588.1, ANAPC10, AC107871.1, CALML4, AC114490.2, ZMYM6, AC138649.1, NIPA1, AC138894.1, CLN3, AC139768.1, AC242426.2, CHDIL, ACADM, ACAP3, ACKR2, RP11-141M3.5, KRBOX1, ACMSD, ACOT9, ACP5, ACPL2, ACSBG1, ACSF2, ACSF3, ACSL1, ACSL3, ACVRI, ADAL, ADAM29, ADAMTS10, ADAMTSL5, ADARB1, ADAT2, ADCK3, ADD3, ADGRG1, ADGRG2, ADH1B, ADIPOR1, ADNP, ADPRH, AGBL5, AGPAT1, AGPAT3, AGR2, AGTR1, AHDC1, AHI1, AHNAK, AIFM1, AIFM3, AIMP2, AK4, AKAP1, AKNAD1, CLCC1, AKR1A1, AKT1, AKT1S1, AKT2, AL139011.2, PEX19, AL157935.2, ST6GALNAC6, AL358113.1, TJP2, AL441992.2, KYAT1, AL449266.1, CLCC1, AL590556.3, LINC00339, CDC42, ALAS1, ALB, ALDH16A1, ALDH1B1, ALDH3A1, ALDH3B2, ALDOA, ALKBH2, ALPL, AMD1, AMICA1, AMN1, AMOTL2, AMYIB, AMY2B, ANAPC10, ANAPC11, ANAPC15, ANG, RNASE4, AL163636.2, ANGEL2, ANGPTL1, ANKMY1, ANKRD11, ANKRD28, ANKRD46, ANKRD9, ANKS3, ANKS3, RP11-127I20.7, ANKS6, ANKZF1, ANPEP, ANXA11, ANXA2, ANXA8L2, AL603965.1, AOC3, AP000304.12, CRYZL1, AP000311.1, CRYZL1, AP000893.2, RAB30, AP001267.5, ATP5MG, AP002495.2, AP003175.1, OR2AT4, AP003419.1, CLCF1, AP005263.1, ANKRD12, AP006621.5, AP006621.1, AP1G1, AP3M1, AP3M2, APBA2, APBB1, APLP2, APOA2, APOL1, APOL3, APTX, ARAPI, STARDIO, ARF4, ARFIP1, ARFIP2, ARFRPI, ARHGAP11A, ARHGAP33, ARHGAP4, ARHGEFIO, ARHGEF3, ARHGEF35, OR2A1-AS1, ARHGEF35, OR2A1-AS1, ARHGEF34P, ARIDIB, ARHGEF35, OR2A20P, OR2A1-AS1, ARHGEF9, ARL1, ARL13B, ARL16, ARL6, ARMC6, ARMC8, ARMCX2, ARMCX5, RP4-769N13.6, ARMCX5-GPRASP2, BHLHB9, ARMCX5-GPRASP2, GPRASP1, ARMCX5-GPRASP2, GPRASP2, ARMCX6, ARNT2, ARPP19, ARRB2, ARSA, ART3, ASB3, GPR75-ASB3, ASCC2, ASNS, ASNS, AC079781.5, ASPSCR1, ASS1, ASUN, ATE1, ATF1, ATF7IP2, ATG13, ATG4D, ATG7, ATG9A, ATM, ATOX1, ATP1B3, ATP2C1, ATP5FIA, ATP5G2, ATP5J, ATP5MD, ATP5PF, ATP6AP2, ATP6VOB, ATP6V1C1, ATP6VID, ATP7B, ATXN1, ATXAIL, ISTI, ATXA3, ATXA7L1, AURKA, AURKB, AXDNDI, B3GALNT1, B3GALT5, AF064860.1, B3GALT5, AF064860.5, B3GNT5, B4GALT3, B4GALT4, B9D1, BACH1, BAIAP2, BANF1, BANF2, BAX, BAZ2A, BBIPJ, BCHE, BCL2L14, BCL6, BCL9L, BCSIL, BDH1, BDKRB2, AL355102.2, BEST1, BEST3, BEX4, BHLHB9, BID, BIN3, BIRC2, BIVM, BIVM-ERCC5, BIVM, BLCAP, BLK, BLOC1S1, RP11-644F5.10, BLOCIS6, AC090527.2, BLOCIS6, RP11-96020.4, BLVRA, BMF, BOLA1, BORCS8-MEF2B, BORCS8, BRCA1, BRDI, BRDT, BRINP3, BROX, BTBD10, BTBD3, BTBD9, BTD, BTF3L4, BTNL9, BUB1B-PAK6, PAK6, BUB3, C10orf68, C11orf1, C11orf48, C11orf54, C11orf54, AP001273.2, C11orf57, C11orf63, C11orf82, C12orf23, C12orf4, C12orf65, C12orf79, C14orf159, C14orf93, C17orf62, C18orf21, C19orf12, C19orf40, C19orf47, C19orf48, C19orf54, C1D, C1GALT1, C1QB, C1QTNF1, C1S, C1orf101, C1orf112, C1orf116, C1orf159, C1orf63, C2, C2, CFB, C20orf27, C21orf58, C2CD4D, C2orf15, LIPT1, MRPL30, C2orf80, C2orf81, C3orf14, C3orf17, C3orf18, C3orf22, C3orf33, AC104472.3, C4orf33, C5orf28, C5orf34, C6orf118, C6orf203, C6orf211, C6orf48, C7orf50, C7orf55, C7orf55-LUC7L2, LUC7L2, C8orf44-SGK3, C8orf44, C8orf59, C9, DAB2, C9orf153, C9orf9, CA5BP1, CASB, CABYR, CALCA, CALCOCO1, CALCOCO2, CALM1, CALM3, CALML4, RP11-315D16.2, CALN, CALU, CANT1, CANX, CAP1, CAPN12, CAPS2, CARD8, CARHSP1, CARNS1, CASCI, CASP3, CASP7, CBFA2T2, CBS, CBY1, CCBL1, CCBL2, RBMXLJ, CCDC12, CCDC126, CCDC14, CCDC149, CCDC150, CCDC169-SOHLH2, CCDC169, CCDC171, CCDC37, CCDC41, CCDC57, CCDC63, CCDC7, CCDC74B, CCDC77, CCDC82, CCDC90B, CCDC91, CCDC92, CCNE1, CCHCR1, CCL28, CCNB1IP1, CCNC, CCND3, CCNG1, CCP110, CCR9, CCT7, CCT8, CD151, CDID, CD200, CD22, CD226, CD276, CD36, CD59, CDC26, CDC42, CDC42SE1, CDC42SE2, CDHR3, CDK1O, CDK16, CDK4, CDKALl, CDKL3, CTD-2410N18.4, CDKN1A, CDKN2A, CDNF, CEBPZOS, CELF1, CEMIP, CENPK, CEP170B, CEP250, CEP57, CEP57L1, CEP63, CERS4, CFL1, CFL2, CFLAR, CGNL1, CHCHD7, CHD1L, CHD8, CHFR, ZNF605, CHIA, CHID1, CHL1, CHM, CHMP1A, CHMP3, RNF103-CHMP3, CHRNA2, CIDEC, CIRBP, CITED1, CKLF-CMTM1, CMTM1, CKMT1B, CLDN12, CTB-13L3.1, CLDNDI, AC021660.3, CLDND1, CPOX, CLHC1, CLIP1, CLUL1, CMC4, MTCP1, CNDP2, CNFN, CNOT1, CNOT6, CNOT7, CNOT8, CNR1, CNR2, CNTFR, CNTRL, COA1, COASY, COCH, COL8A1, COLCA1, COLEC11, COMMD3-BMI1, BMI1, COPS5, COPS7B, COQ8A, CORO6, COTLI, COX14, RP4-60503.4, COX7A2, COX7A2L, COX7B2, CPA4, CPA5, CPEBI, CPNEI, AL109827.1, RBM12, CPNEI, RP1-309K20.6, RBM12, CPNE3, CPSF3L, CPT1C, CREB3L2, CREM, CRP, CRYZ, CS, AC073896.1, CS, RP11-977G19.10, CSAD, CSDE1, CSF2RA, CSGALNACT1, CSK, CSNK2A1, CSRNP2, CT45A4, CT45A4, CT45A5, CT45A6, CTBP2, CTCFL, CTD-2116N17.1, KIAA0101, CTD-2349B8.1, SYT17, CTD-2528L19.4, ZNF607, CTD-2619J13.8, ZNF497, CTNNA1, CTNNBIP1, CTNND1, CTPS2, CTSB, CTSL, CTTN, CUL2, CUL9, CWC15, CXorf40B, CYB561A3, CYBC1, CYLD, CYP11A1, CYP2R1, CYP4B1, CYP4F22, DAG1, DAGLB, KDELR2, DARS, DBNL, DCAF11, DCAF8, PEX19, DCLRE1C, DCTD, DCTN1, DCTN4, DCUN1D2, DDR1, DDX11, DDX19B, AC012184.2, DDX19B, RP11-529K10.3, DDX25, DDX39B, ATP6V1G2-DDX39B, SNORD84, DDX42, DDX60L, DEDD, DEDD2, DEFA1, DEFA1B, DEFA1B, DEFA3, DENND1C, DENND2A, DENND4B, DET1, DGKA, DGKZ, DGLUCY, DHRS4L2, DHRS9, DHX40, DIABLO, AC048338.1, DIAPH1, DICER1, DKKL1, DLG1, DLG3, DLST, DMC1, DMKN, DMTF1, DMTN, DNAJC14, DNAJC19, DNAL1, DNASEIL1, DNMT3A, DOC2A, DOCK8, DOK1, DOPEY1, DPAGT1, DPP8, DRAM2, DRD2, DROSHA, DSN1, DTNA, DTX2, DTX3, DUOX1, DUOXA1, DUS2, DUSP10, DUSP13, DUSP18, DUSP22, DYDC1, DYDC2, DYNLL1, DYNLT1, DYRK1A, DYRK2, DYRK4, RP11-500M8.7, DZIP1L, E2F6, ECHDC1, ECSIT, ECT2, EDC3, EDEM1, EDEM2, MMP24-AS1, RP4-61404.1H, EEFIAKNMT, EEF1D, EFEMP1, EFHCl, EGFL7, EHF, EI24, EIF1AD, EIF2B5, EIF4G1, EIF2B5, POLR2H, EIF3E, EIF3K, EIF4E3, EIF4G1, ELF1, ELMO2, ELMOD1, AP000889.3, ELMOD3, ELOC, ELOF1, ELOVL, ELOVL7, ELP1, ELP6, EML3, EMP3, ENC1, ENDOV, ENO1, ENPP5, ENTHD2, ENTPD6, EP400NL, EPB41L1, EPDR1, NME8, EPHX1, EPM2A, EPN1, EPN2, EPN3, EPS8L2, ERBB3, ERC1, ERCC1, ERG, ERI2, ERI2, DCUNID3, ERLIN2, ERMARD, ERRFIl, ESR2, RP11-544I20.2, ESRRA, ESRRB, ESRRG, ETFA, ETFRF1, ETVJ, ETV4, ETV7, EVA1A, EVC2, EVX1, EXD2, EXO5, EXOC1, EXOC2, FAAP24, FABP6, FADS1, FADS2, FAHD2B, FAM107B, FAM111A, FAM111B, FAM114A1, FAM114A2, FAM115C, FAM115C, FAM115D, FAM120B, FAM133B, FAM135A, FAM153A, FAM153B, FAM154B, FAM156A, FAM156B, FAM168B, FAM172A, FAM182B, FAM192A, FAM19A2, FAM200B, FAM220A, FAM220A, AC009412.1, FAM222B, FAM227B, FAM234A, AC004754.1, FAM3C, FAM45A, FAM49B, FAM60A, FAM63A, FAM81A, FAM86B1, FAM86B2, FANCI, FANK1, FAR2, FAXC, FAXDC2, FBF1, FBH1, FBXL4, FBXO18, FBXO22, FBXO31, FBXO41, FBXO44, FBXO45, FBXW9, FCHOI, FCHSD2, FDFT1, FDPS, FER, FETUB, FGD4, FGF1, FGFR1, FGFRL1, FGL1, FHL2, FIBCD1, FIGNL1, FIGNL1, DDC, FKBP5, FKRP, FLRT2, FLRT3, FMC1, LUC7L2, FMC1-LUC7L2, FNDC3B, FOLH1, FOLRI, FOXPI, FOXK1, FOXMl, FOXO1, FOXP4, AC097634.4, FOXREDI, FPR1, FPR2, FRG1B, FRS2, FTO, FTSJ1, FUK, FUTIO, FUT3, FUT6, FXYD3, FZD3, G2E3, GAA, GABARAPLI, GABPB1, GABRAS, GAL3ST1, GALE, GALNT1l, GALNT14, GALNT6, GAPVD1, GARNL3, GAS2L3, GAS8, GATA1, GATA2, GATA4, GBA, GCNT1, GDPD2, GDPD5, GEMIN7, MARK4, GEMIN8, GGA3, GGACT, AL356966.1, GGPS1, GHRL, GID8, GIGYF2, GIMAP8, GIPCI, GJB1, GJB6, GLBIL, GLIJ, GLT8D1, GMFG, GMPR2, GNAI2, GNAQ, GNB1, GNB2, GNE, GNG2, GNGT2, GNPDA1, GNPDA2, GOLGA3, CHFR, GOLGA4, GOLPH3L, GOLTIB, GPBPILI, GPERI, GPR116, GPR141, EPDR1, GPR155, GPR161, GPR56, GPR63, GPR75-ASB3, ASB3, GPR85, GPSM2, GRAMDIB, GRBIO, GRB7, GREM2, GRIA2, GSDMB, GSE1, GSN, GSTA4, GSTZ1, GTDC1, GTF2H1, GTF2H4, VARS2, GTF3C2, GUCY1A3, GUCYIB3, GUK1, GULP1, GYPC, GYS1, GZF1, HAGH, HA02, HAPLN3, HAVCR1, HAX1, HBG2, AC104389.4, HBG2, AC104389.4, HBE1, HBG2, AC104389.4, HBEI, OR51B5, HBG2, HBE1, AC104389.28, HBS1L, HCFC1R1, HCK, HDAC2, HDAC6, HDAC7, HDLBP, HEATR4, HECTD4, HEXIM2, HHAT, HHATL, CCDC13, HINFP, HIRA, C22orf39, HIVEP3, HJV, HKRI, HLF, HMBOXI, HMGA1, HMGB3, HMGCR, HMGN4, HMOX2, HNRNPC, HNRNPD, HNRNPH1, HNRNPH3, HNRNPR, HOMER3, HOPX, HOXA3, HOXB3, HOXB3, HOXB4, HOXC4, HOXD3, HOXD3, HOXD4, HPCAL1, HPS4, HPS5, HRHJ, HS3ST3A1, HSH2D, HSP90AA1, HSPD1, HTT, HUWE1, HYOU1, IAHJ, ICA1L, ICAM2, ICE2, ICK, IDH2, IDH3G, IDS, IFI27, IFI44, IFT20, IFT22, IFT88, IGF2, INS-IGF2, IGF2BP3, IGFBP6, IKBKAP, IKBKB, IL11, IL18BP, IL18RAP, ILIRAP, ILIRLI, IL18R1, ILIRN, IL32, IL4I1, NUP62, AC011452.1, IL4I1, NUP62, CTC-326K19.6, IL6ST, ILVBL, IMALPIL, IMPDHI, INCA1, ING1, INIP, INPPI, INPP5J, INPP5K, INSIG2, INTS11, INTS12, INTS14, IP6K2, IP6K3, IPO11, LRRC70, IQCE, IQGAP3, IRAK4, IRF3, IRF5, IRF6, ISG20, ISTI, ISYNA1, ITFG2, ITGBIBPI, ITGB7, ITIH4, RP5-966M1.6, ITPRIPL1, JADE1, JAK2, JARID2, JDP2, KANK1, KANK1, RP11-31F19.1, KANK2, KANSL1L, KAT6A, KBTBD2, KBTBD3, KCNAB2, KCNE3, KCNG1, KCNJ16, KCNJ9, KCNMB2, AC117457.1, LINC01014, KCTD20, KCTD7, RABGEF1, KDM1B, KDM4A, AL451062.3, KHNYN, KIAA0040, KIAA0125, KIAA0196, KIAA0226L, PPPIR2P4, KIAA0391, KIAA0391, AL121594.1, KIAA0391, PSMA6, KIAA0753, KIAA0895, KIAA0895L, KIAA1191, KIAA1407, KIAA1841, C2orf74, KIF12, KIF14, KIF27, KIF9, KIFC3, KIN, KIRREL1, KITLG, KLC, APOPT, AL139300.1, KLC4, KLHDC4, KLHDC8A, KLHL13, KLHL18, KLHL2, KLHL24, KLHL7, KLK11, KLK2, KLK5, KLK6, KLK7, KNOP1, KRBA2, AC135178.2, KRBA2, RP11-849F2.7, KRIT1, KRT15, KRT8, KTNI, KXDI, KYAT3, RBMXL1, KYNU, L3MBTL1, LACCl, LARGE, LARP4, LARP7, LAT2, LBHDI, LCA5, LCA5L, LCTL, LEPROTLI, LGALS8, LGALS9C, LGMN, LHFPL2, LIG4, LIMCH, LIMK2, LIMS2, LINC00921, ZNF263, LIPF, LLGL2, LMAN2L, LMCD, LMFI, RPLl-161M6.2, LMO1, LMO3, LOXHDI, LPAR1, LPAR2, LPAR4, LPAR5, LPAR⁶, LPHNI, LPIN2, LPIN3, LPP, LRFN5, LRIFI, LRAMP, LRRC14, LRRC20, LRRC24, C8orf82, LRRC39, LRRC42, LRRC48, LRRC4C, LRRC8A, LRRC8B, LRRD1, LRTOMT, LRTOMT, AP000812.5, LSM7, LTB4R, LTBP3, LUC7L2, FMCI-LUC7L2, LUC7L3, LUZPJ, LYG1, LYL1, LYPD4, LYPD6B, LYRMl, LYRMS, LYSMD4, MACC1, MADIL1, MAD1L1, AC069288.1, MAEA, MAFF, MAFG, MAFK, MAGEA12, CSAG4, MAGEA2, MAGEA2B, MAGEA4, MAGEB1, MAGOHB, MAN2A2, MANBAL, MAOB, MAP2K3, MAP3K7CL, MAP3K8, MAP7, MAP9, MAPK6, MAPK7, MAPK8, MAPKAPI, 10-Mar, 7-Mar, 8-Mar, MARK2, MASPI, MATK, MATR3, MATR3, SNHG4, MB, MBD5, MBNL1, MBOAT7, MCC, MCFD2, MCM9, MCOLN3, MCRSI, MDCl, MDGA2, MDH2, MDM2, ME1, MEAK7, MECR, MED4, MEF2A, MEF2B, BORCS8-MEF2B, MEF2BNB-MEF2B, MEF2B, MEF2BNB, MEF2C, MEF2D, MEGF10, MEI1, MEIS2, MELK, MET, METTL13, METTL23, MFF, MFN2, MFSD2A, MGST3, MIB2, MICAL1, MICAL3, MICOSO, NBL1, MICOS1O-NBLJ, MID1, MINA, MINOS1-NBL1, MINOS1, MIOS, MIPOL1, MIS12, MKLNJ, MKNKI, MKNKI, MOB3C, MLF2, MLH1, MMP17, MOBP, MOCS1, MOGS, MOK, MORF4L1, MPC1, MPC2, MPG, MPI, MPP1, MPP2, MPPEI, MPST, MRAS, MRO, MROH1, MROH7-TTC4, MROH7, MRPL14, MRPL24, MRPL33, BABAM2, MRPL33, BRE, MRPL47, MRPL48, MRPL55, MRRF, MRTFA, MRTFB, MRVIJ, MS4A1, MS4A15, MS4A3, MS4A6E, MS4A7, MS4A14, MSANTD3, MSANTD4, MSH5, MSH5-SAPCDl, MSL2, MSRB3, MSS51, MTCPI, CMC4, MTERF, MTERFJ, MTERF3, MTERFD2, MTERFD3, MTF2, MTG2, MTHFD2, MTHFD2L, MTIF2, MTIF3, MTMRI0, MTRF1, MTRR, MTUS2, MUTYH, MVK, MX1, MX2, MYH10, MYL12A, MYB, MYD88, MYLS, MYLIP, MYNN, MYOl5A, MYOiB, MYOM2, MZFJ, N4BP2L2, NAA60, NAB1, NAEl, NAGK, NAPILI, NAPIL4, NAPG, NARFL, NARG2, NAT, NATI0, NBPF11, WI2-3658N16.1, NBPF12, NBPF15, NBPF24, NBPF6, NBPF9, NBRI, NCAPG2, NCBP2, NCEHJ, NCOA1, NCOA4, NDCl, NDRG1, NDRG2, NDRG4, NDSTI, NDUFAF6, NDUFB2, NDUFCJ, NDUFS1, NDUFS8, NDUFVJ, NEDDI, NEIL1, NEIL2, NEK10, NEK11, NEK6, NEK9, NELFA, NEU4, NFAT5, NFE2, NFE2L2, AC019080.1, NFRKB, NFYA, NFYC, NIF3L1, NIPA2, NKIRASI, NKX2-1, NLRC3, NME1, NME1-NME2, NME2, NME1-NME2, NME2, NME4, NME6, NME9, NOD1, NOL10, NOL8, NONO, NPAS1, NPIPA8, RP11-1212A22.1, NPIPB3, NPIPB4, NPIPB9, NPL, NPM1, NPPA, NQO2, NRIH3, NR2C2, NR2F2, NR4A1, NRDC, NREP, NRFJ, NRG4, NRIP1, NSD2, NSDHL, NSG1, NSMCE2, NSRP1, NT5C2, NTF4, NTMTJ, NTNG2, NUBP2, NUCB2, NUDT1, NUDT2, NUDT4, NUF2, NUMBL, NUP50, NUP54, NUP85, NVL, NXF1, NXPE1, NXPE3, OARD1, OAT, OAZ2, OCIAD1, OCLN, ODF2, OGDHL, OGFOD2, AC026362.1, OGFOD2, RP11-197N18.2, OLA1, OPRLI, OPTN, OR2H1, ORAI2, ORMDL1, ORMDL2, ORMDL3, OSBPL2, OSBPL3, OSBPL5, OSBPL9, OSER1, OSGIN1, OSR2, P2RX4, P2RY2, P2RY6, P4HA2, PABPC1, PACRGL, PACSIN3, PADI1, PAIP2, PAK1, PAK3, PAK4, PAK7, PALB2, PANK2, PAQR⁶, PARP11, PARVG, PASK, PAX6, PBRM1, PBXIPl, PCBP3, PCBP4, AC115284.1, PCBP4, RP11-155D18.14, RP11-155D18.12, PCGF3, PCGF5, PCNP, PCSK9, PDCDIO, PDCD6, AHRR, PDDCl, PDGFRB, PDIA6, PDIKIL, PDLIM7, PDP1, PDPK1, PDPN, PDZD11, PEA15, PEX2, PEX5, PEX5L, PFKM, PFN4, PGAP2, PGAP2, AC090587.2, PGAP3, PGM3, PGPEP1, PHB, PHC2, PHF20, PHF21A, PHF23, PHKB, PHLDB1, PHOSPHO1, PHOSPHO2, KLHL23, PI4 KB, PIAS2, PICALM, PIF1, PIGN, PIGO, PIGT, PIK3CD, PILRB, STAG3L5P-PVRIG2P-PILRB, PIP5K1B, PIR, PISD, PIWIL4, FUT4, PKD2, PKIA, PKIG, PKM, PKN2, PLA1A, PLA2G2A, PLA2G5, PLA2G7, PLAC8, PLAGL1, PLD1, PLD3, PLEKHAI, PLEKHA2, PLEKHA6, PLEKHG5, PLIN1, PLSI, PLS3, PLSCRI, PLSCR2, PLSCR4, PLXNBI, PLXNB2, PMP22, PMS1, PNISR, PNKP, AKTIS1, PNMT, PNPLA4, PNPLA8, PNPO, PNRCI, POCIB, POFUT1, POLB, POLD1, POLH, POLI, POLL, POLRIB, POM121, POM121C, AC006014.7, POM121C, AC211429.1, POMC, POMT1, POP1, PORCN, POU5F1, PSORSIC3, PPARD, PPARG, PPHLNJ, PPIL3, PPIL4, PPM1A, PPM1B, AC013717.1, PPP1CB, PPP1R11, PPP1R13L, PPP1R26, PPP1R9A, PPP2R2B, PPP3CA, PPP6R1, PPP6R3, PPT2, PPT2-EGFL8, EGFL8, PPWD1, PRDM2, PRDM8, PRELID3A, PREPL, PRICKLE1, PRKAGI, PRMT2, PRMTS, PRMT7, PROM1, PRPSI, PRPSAP2, PRR14L, PRR15L, PRR5, PRR5-ARHGAP8, PRR5L, PRR7, PRRC2B, PRRT4, PRSS50, PRSS45, PRSS44, PRUNE, PRUNE1, PSEN1, PSMA2, PSMF1, PSORSICI, PSPH, PSRC1, PTBP3, PTHLH, PTK2, PTPDCI, PTPRM, PUF60, PUM2, PUS1, PUSIO, PXN, PXYLP1, PYCR1, QRICH1, R3HCCIL, R3HDM2, RAB17, RAB23, RAB3A, RAB3D, TMEM205, RAB4B-EGLN2, EGLN2, AC008537.1, RAB5B, RAB7L1, RABL2A, RABL2B, RABL5, RACGAPI, RAD17, RAD51L3-RFFL, RAD51D, RAD52, RAE1, RAI14, RAI2, RALBP1, RAN, RANGAP1, RAPIA, RAPIB, RAPIGAP, RAPGEF4, RAPGEFL1, RASGRP2, RASSF1, RBCKI, RBMI2B, RBM14, RBM4, RBM14-RBM4, RBM23, RBM4, RBM14-RBM4, RBM47, RBM7, AP002373.1, RBM7, RP11-212D19.4, RBMS2, RBMYIE, RBPJ, RBPMS, RBSN, RCBTB2, RCC1, RCC1, SNHG3, RCCD1, RECQL, RELL2, REPIN1, AC073111.3, REPIN1, ZNF775, RER1, RERE, RFWD3, RFX3, RGL2, RGMB, RGSI1, RGS3, RGS5, AL592435.1, RHBDDI, RHNOI, TULP3, RHOC, AL603832.3, RHOC, RP11-426L16.10, RHOH, RIC8B, RIMKLB, RIN1, RIPK2, RIT1, RLIM, RNASE4, ANG, AL163636.6, RNASEK, RNASEK-C₁₇orf49, RNFI11, RNF123, RNF13, RNF14, RNF185, RNF216, RNF24, RNF32, RNF34, RNF38, RNF4, RNF44, RNH1, RNMT, RNPSI, R060, ROPNI, ROPNIB, ROR2, RP1-102H19.8, C6orf163, RP1-283E3.8, CDKIIA, RP11-120M18.2, PRKAR1A, RP11-133K10.2, PAK6, RP11-164J13.1, CAPN3, RP11-21J18.1, ANKRD12, RP11-322E1.6, INO80C, RP11-337C18.10, CHDIL, RP11-432B6.3, TRIM59, RP11-468E2.4, IRF9, RP11-484M3.5, UPKIB, RP11-517H2.6, CCR⁶, RP11-613M10.9, SLC25A51, RP11-659G9.3, RAB30, RP11-691N7.6, CTNND, RP11-849H4.2, RPI-896J0.3, NKX2-1, RP11-96020.4, SQRDL, RP11-986E7.7, SERPINA3, RP4-769N13.6, GPRASP1, RP4-769N13.6, GPRASP2, RP4-798P15.3, SEC16B, RP5-10211I20.4, ZNF410, RP6-109B7.3, FLJ27365, RPE, RPH3AL, RPL15, RPL17, RPL17-C18orf32, RPL17, RPL23A, RPL36, HSDI1BIL, RPP38, RPS20, RPS27A, RPS3A, RPS6KA3, RPS6KC, RPS6KL1, RPUSD1, RRAGD, RRAS2, RRBPJ, RSLID1, RSRC2, RSRPI, RUBCNL, RUNXITI, RUVBL2, RWDDI, RWDD4, SOOA13, AL162258.1, SOOA13, RP1-178F15.5, SOOA16, SI00A4, S100A3, S100A6, SOOPBP, SAA1, SACMIL, SAMD4B, SARlA, SARAF, SARNP, RP11-762I7.5, SCAMPS, SCAP, SCAPER, SCFD, SCGB3A2, SCIN, SCMLI, SCNNID, SCO2, SCOC, SCRN1, SDC2, SDC4, SEC13, SEC14L1, SEC14L2, SEC22C, SEC23B, SEC24C, SEC61G, SEMA4A, SEMA4C, SEMA4D, SEMA6C, SENP7, SEPPI, 11-Sep, 2-Sep, SERGEF, AC055860.1, SERP1, SERPINA1, SERPINA5, SERPINB6, SERPING1, SERPINHI, SERTAD3, SETD5, SFMBT1, AC096887.1, SFTPAI, SFTPA2, SFXN2, SGCD, SGCE, SGK3, SGK3, C8orf44, SH2B1, SH2D6, SH3BP1, Z83844.3, SH3BP2, SH3BP5, SH3D19, SH3YL1, SHC1, SHISAS, SHMT1, SHMT2, SHOC2, SHROOM1, SIGLEC5, SIGLEC14, SIL1, SIN3A, SIRT2, SIRT6, SKP1, STAT4, AC104109.3, SLAIN1, SLCI0A3, SLC12A9, SLC14A1, SLC16A6, SLCIA2, SLCIA6, SLC20A2, SLC25A18, SLC25A19, SLC25A22, SLC25A25, SLC25A29, SLC25A30, SLC25A32, SLC25A39, SLC25A44, SLC25A45, SLC25A53, SLC26A11, SLC26A4, SLC28A1, SLC29A1, SLC2A14, SLC2A5, SLC2A8, SLC35B2, SLC35B3, SLC35C2, SLC37A1, SLC38A1, SLC38A11, SLC39A13, SLC39A14, SLC41A3, SLC44A3, SLC4A7, SLC4A8, SLC5A10, SLC5A11, SLC6A1, SLC6A12, SLC6A9, SLC7A2, SLC7A6, SLC7A7, SLCOIA2, SLCO1C1, SLCO2B1, SLFNI1, SLFN12, SLFNL1, SLMOI, SLTM, SLU7, SMAD2, SMAP2, SMARCA2, SMARCE1, AC073508.2, SMARCE1, KRT222, SMC6, SMG7, SMIM22, SMOX, SMPDL3A, SMTN, SMU1, SMUG1, SNAP25, SNCA, SNRK, SNRPC, SNRPD1, SNRPD2, SNRPN, SNRPN, SNURF, SNUPN, SNXI1, SNX16, SNX17, SOATI, SOHLH2, CCDC169-SOHLH2, CCDC169, SORBS1, SORBS2, SOX5, SP2, SPART, SPATA20, SPATA21, SPATS2, SPATS2L, SPDYE2, SPECC1, SPECCIL, SPECCIL-ADORA2A, SPECCIL-ADORA2A, ADORA2A, SPEG, SPG20, SPG21, SPIDR, SPIN1, SPOCD1, SPOP, SPRR2A, SPRR2B, SPRR2E, SPRR2B, SPRR2F, SPRR2D, SPRR3, SPRY1, SPRY4, SPTBN2, SR^{C}, SRGAPI, SRP68, SRSF1l, SSXJ, SSX2IP, ST3GAL4, ST3GAL6, ST5, ST6GALNAC6, ST7L, STAC3, STAG1, STAG2, STAMBP, STAMBPLI, STARD3NL, STAT6, STAU1, STAU2, AC022826.2, STAU2, RP11-463D19.2, STEAP2, STEAP3, STIL, STK25, STK33, STK38L, STK40, STMN1, STON1, STON1-GTF2AIL, STRAP, STRBP, STR^{C}, AC011330.5, STR^{C}, CATSPER2, STRC, CATSPER2, AC011330.5, STRC, STRCPI, STT3A, STX16-NPEPLl, NPEPL1, STX5, STX6, STX8, STXBP6, STYK1, SULTIAl, SULTIA2, SUMF2, SUN1, SUN2, SUN2, DNAL4, SUOX, SUPT6H, SUV39H2, SV2B, SYBU, SYNCRIP, SYNJ2, SYT1, SYTL4, TAB2, TACC1, TADA2B, TAFIC, TAF6, AC073842.2, TAF6, RP11-506M12.1, TAF9, TAGLN, TANK, TAPSARI, PSMB9, TAPT1, TATDN1, TAZ, TBC1D1, TBC1D12, HELLS, TBC1D15, TBCID3H, TBCID3G, TBC1D5, TBC1D5, SATB1, TBCA, TBCEL, TBCEL, AP000646.1, TBL1XR1, TBP, TBX5, TBXAS1, TCAF1, TCEA2, TCEAL4, TCEAL8, TCEAL9, TCEANC, TCEB1, TCF19, TCF25, TCF4, TCP1, TCPIOL, AP000275.65, TCP1l, TCPI1L2, TCTNI, TDG, TDP1, TDRD7, TEAD2, TECR, TENC1, TENT4A, TEX264, TEX30, TEX37, TFDP1, TFDP2, TFEB, TFG, TFP1, TF, TFPI, TGIFI, THAP6, THBS3, THOCS, THRAP3, THUMPD3, TIAL1, TIMM9, TIMP1, TIRAP, TJAP1, TJP2, TK2, TLDC1, TLE3, TLE6, TLN1, TLRIO, TM9SF1, TMBIM1, TMBIM4, TMBIM6, TMC6, TMCCI, TMCO4, TMEM126A, TMEM139, TMEM150B, TMEM155, TMEM161B, TMEM164, TMEM168, TMEM169, TMEM175, TMEM176B, TMEM182, TMEM199, CTB-96E2.3, TMEM216, TMEM218, TMEM230, TMEM263, TMEM45A, TMEM45B, TMEM62, TMEM63B, TMEM66, TMEM68, TMEM98, TMEM9B, TMPRSSIID, TMPRSS5, TMSB15B, TMTC4, TMUB2, TMX2-CTNND1, RP11-691N7.6, CTNND1, TNFAIP2, TNFAIP8L2, SCNM1, TNFRSF10C, TNFRSF19, TNFRSF8, TNFSF12-TNFSF13, TNFSF12, TNFSF13, TNFSF12-TNFSF13, TNFSF13, TNIP1, TNK2, TNNT1, TNRC18, TNS3, TOB2, TOMILI, TOPIMT, TOP3B, TOX2, TP53, RP11-199F11.2, TP5311, TP53INP2, TPCN1, TPM3P9, AC022137.3, TPT1, TRA2B, TRAF2, TRAF3, TRAPPC12, TRAPPC3, TREH, TREXI, TREX2, TRIB2, TRIM3, TRIM36, TRIM39, TRIM46, TRIM6, TRIM6-TRIM34, TRIM6-TRIM34, TRIM34, TRIM66, TRIM73, TRITI, TRMTJOB, TRMT2B, TRMT2B-AS1, TRNT1, TRO, TROVE2, TRPS1, TRPTI, TSC2, TSGA10, TSPANJ4, TSPAN3, TSPAN4, TSPAN5, TSPAN6, TSPAN9, TSPO, TTCJ2, TTC23, TTC3, TTC39A, TTC39C, TTLLI, TTLL7, TTPAL, TUBDI, TWNK, TXNL4A, TXNL4B, TXNRDJ, TYK2, U2AF1, UBA2, UBA52, UBAP2, UBE2D2, UBE2D3, UBE2E3, UBE2I, UBE2J2, UBE3A, UBL7, UBXNJJ, UBXN7, UGDH, UGGTI, UGP2, UMADJ, AC007161.3, UNC45A, UQCCJ, URGCP-MRPS24, URGCP, USMG5, USP16, USP21, USP28, USP3, USP33, USP35, USP54, USP9Y, USPLI, UTP15, VARS2, VASH2, VAV3, VDACI, VDAC2, VDR, VEZT, VGF, VILI, VILL, VIPRI, VPS29, VPS37C, VPS8, VPS9D1, VRK2, VWAI, VWA5A, WARS, WASFI, WASHC5, WBP5, WDHDI, WDPCP, WDR37, WDR53, WDR⁶, WDR72, WDR74, WDR81, WDR86, WDYHVI, WFDC3, WHSCI, WIPF, WSCD2, WWP2, XAGEIA, XAGEIB, XKR9, XPNPEPI, XRCC3, XRN2, XXYLTI, YIFIA, YIFIB, YIPF1, YIPF5, YPEL5, YWHAB, YWHAZ, YY1AP1, ZBTBI, ZBTB14, ZBTB18, ZBTB20, ZBTB21, ZBTB25, ZBTB33, ZBTB34, ZBTB38, ZBTB43, ZBTB49, ZBTB7B, ZBTB7C, ZBTB8OS, ZC3H11A, ZBED6, ZC3H13, ZCCHC17, ZCCHC7, ZDHHCII, ZDHHC13, ZEB2, ZFANDS, ZFAND6, ZFPI, ZFP62, ZFX, ZFYVE16, ZFYVE19, ZFYVE20, ZFYVE27, ZHX2, AC016405.1, ZHX3, ZIKI, ZIM2, PEG3, ZKSCANI, ZKSCAN3, ZKSCAN8, ZMAT3, ZMAT5, ZMIZ2, ZMYM6, ZMYNDII, ZNFO, AC026786.1, ZNFJ33, ZNF146, ZNF16, ZNFJ77, ZNF18, ZNF200, ZNF202, ZNF211, ZNF219, ZNF226, ZNF227, ZNF23, AC010547.4, ZNF23, AC010547.9, ZNF239, ZNF248, ZNF25, ZNF253, ZNF254, ZNF254, AC092279.1, ZNF263, ZNF274, ZNF275, ZNF28, ZNF468, ZNF283, ZNF287, ZNF3, ZNF320, ZNF322, ZNF324B, ZNF331, ZNF334, ZNF34, ZNF350, ZNF385A, ZNF395, FBXO16, ZNF415, ZNF418, ZNF43, ZNF433-AS1, AC008770.4, ZNF438, ZNF444, ZNF445, ZNF467, ZNF480, ZNF493, ZNF493, CTD-2561J22.3, ZNF502, ZNF507, ZNF512, AC074091.1, ZNF512, RP11-158I13.2, ZNF512B, ZNF512B, SAMD10, ZNF521, ZNF532, ZNF544, AC020915.5, ZNF544, CTD-3138B18.4, ZNF559, ZNF177, ZNF562, ZNF567, ZNF569, ZNF570, ZNF571-AS1, ZNF540, ZNF577, ZNF580, ZNF581, ZNF580, ZNF581, CCDC106, ZNF600, ZNF611, ZNF613, ZNF615, ZNF619, ZNF620, ZNF639, ZNF652, ZNF665, ZNF667, ZNF668, ZNF671, ZNF682, ZNF687, ZNF691, ZNF696, ZNF701, ZNF706, ZNF707, ZNF714, ZNF717, ZNF718, ZNF720, ZNF721, ZNF730, ZNF763, ZNF780B, AC005614.5, ZNF782, ZNF786, ZNF79, ZNF791, ZNF81, ZNF83, ZNF837, ZNF839, ZNF84, ZNF845, ZNF846, ZNF865, ZNF91, ZNF92, ZNHIT3, ZSCAN21, ZSCAN25, ZSCAN30, and ZSCAN32.

In some embodiments, the gene encoding a target sequence comprises the HTT gene. In some embodiments, the gene encoding a target sequence comprises the MYB gene. In some embodiments, the gene encoding a target sequence comprises the SMN2 gene. In some embodiments, the gene encoding a target sequence comprises the FOXM1 gene.

Exemplary genes that may be modulated by the compounds of Formula (I), (II), (III), (IV) described herein may also include, inter alia, AC005258.1, AC005943.1, AC007849.1, AC008770.2, AC010487.3, AC011477.4, AC012651.1, AC012531.3, AC034102.2, AC073896.4, AC104472.3, AL109811.3, AL133342.1, AL137782.1, AL157871.5, AF241726.2, AL355336.1, AL358113.1, AL360181.3, AL445423.2, AL691482.3, AP001267.5, RF01169, and RF02271.

The compounds described herein may further be used to modulate a sequence comprising a particular splice site sequence, e.g., an RNA sequence (e.g., a pre-mRNA sequence). In some embodiments, the splice site sequence comprises a 5′ splice site sequence. In some embodiments, the splice site sequence comprises a 3′ splice site sequence. Exemplary gene sequences and splice site sequences (e.g., 5′ splice site sequences) include AAAgcaaguu (SEQ ID NO: 1), AAAguaaaaa (SEQ ID NO: 2), AAAguaaaau (SEQ ID NO: 3), AAAguaaagu (SEQ ID NO: 4), AAAguaaaua (SEQ ID NO: 5), AAAguaaaug (SEQ ID NO: 6), AAAguaaauu (SEQ ID NO: 7), AAAguaacac (SEQ ID NO: 8), AAAguaacca (SEQ ID NO: 9), AAAguaacuu (SEQ ID NO: 10), AAAguaagaa (SEQ ID NO: 11), AAAguaagac (SEQ ID NO: 12), AAAguaagag (SEQ ID NO: 13), AAAguaagau (SEQ ID NO: 14), AAAguaagca (SEQ ID NO: 15), AAAguaagcc (SEQ ID NO: 16), AAAguaagcu (SEQ ID NO: 17), AAAguaagga (SEQ ID NO: 18), AAAguaaggg (SEQ ID NO: 19), AAAguaaggu (SEQ ID NO: 20), AAAguaagua (SEQ ID NO: 21), AAAguaaguc (SEQ ID NO: 22), AAAguaagug (SEQ ID NO: 23), AAAguaaguu (SEQ ID NO: 24), AAAguaaucu (SEQ ID NO: 25), AAAguaauua (SEQ ID NO: 26), AAAguacaaa (SEQ ID NO: 27), AAAguaccgg (SEQ ID NO: 28), AAAguacuag (SEQ ID NO: 29), AAAguacugg (SEQ ID NO: 30), AAAguacuuc (SEQ ID NO: 31), AAAguacuug (SEQ ID NO: 32), AAAguagcuu (SEQ ID NO: 33), AAAguaggag (SEQ ID NO: 34), AAAguaggau (SEQ ID NO: 35), AAAguagggg (SEQ ID NO: 36), AAAguaggua (SEQ ID NO: 37), AAAguaguaa (SEQ ID NO: 38), AAAguauauu (SEQ ID NO: 39), AAAguauccu (SEQ ID NO: 40), AAAguaucuc (SEQ ID NO: 41), AAAguaugga (SEQ ID NO: 42), AAAguaugua (SEQ ID NO: 43), AAAguaugug (SEQ ID NO: 44), AAAguauguu (SEQ ID NO: 45), AAAguauugg (SEQ ID NO: 46), AAAguauuuu (SEQ ID NO: 47), AAAgucagau (SEQ ID NO: 48), AAAgucugag (SEQ ID NO: 49), AAAgugaaua (SEQ ID NO: 50), AAAgugagaa (SEQ ID NO: 51), AAAgugagac (SEQ ID NO: 52), AAAgugagag (SEQ ID NO: 53), AAAgugagau (SEQ ID NO: 54), AAAgugagca (SEQ ID NO: 55), AAAgugagcu (SEQ ID NO: 56), AAAgugaggg (SEQ ID NO: 57), AAAgugagua (SEQ ID NO: 58), AAAgugaguc (SEQ ID NO: 59), AAAgugagug (SEQ ID NO: 60), AAAgugaguu (SEQ ID NO: 61), AAAgugcguc (SEQ ID NO: 62), AAAgugcuga (SEQ ID NO: 63), AAAguggguc (SEQ ID NO: 64), AAAguggguu (SEQ ID NO: 65), AAAgugguaa (SEQ ID NO: 66), AAAguguaug (SEQ ID NO: 67), AAAgugugug (SEQ ID NO: 68), AAAguguguu (SEQ ID NO: 69), AAAguuaagu (SEQ ID NO: 70), AAAguuacuu (SEQ ID NO: 71), AAAguuagug (SEQ ID NO: 72), AAAguuaugu (SEQ ID NO: 73), AAAguugagu (SEQ ID NO: 74), AAAguuugua (SEQ ID NO: 75), AACguaaaac (SEQ ID NO: 76), AACguaaagc (SEQ ID NO: 77), AACguaaagg (SEQ ID NO: 78), AACguaagca (SEQ ID NO: 79), AACguaaggg (SEQ ID NO: 80), AACguaaguc (SEQ ID NO: 81), AACguaagug (SEQ ID NO: 82), AACguaaugg (SEQ ID NO: 83), AACguaguga (SEQ ID NO: 84), AACguaugua (SEQ ID NO: 85), AACguauguu (SEQ ID NO: 86), AACgugagca (SEQ ID NO: 87), AACgugagga (SEQ ID NO: 88), AACgugauuu (SEQ ID NO: 89), AACgugggau (SEQ ID NO: 90), AACgugggua (SEQ ID NO: 91), AACguguguu (SEQ ID NO: 92), AACguuggua (SEQ ID NO: 93), AAGgcaaauu (SEQ ID NO: 94), AAGgcaagag (SEQ ID NO: 95), AAGgcaagau (SEQ ID NO: 96), AAGgcaagcc (SEQ ID NO: 97), AAGgcaagga (SEQ ID NO: 98), AAGgcaaggg (SEQ ID NO: 99), AAGgcaagug (SEQ ID NO: 100), AAGgcaaguu (SEQ ID NO: 101), AAGgcacugc (SEQ ID NO: 102), AAGgcagaaa (SEQ ID NO: 103), AAGgcaggau (SEQ ID NO: 104), AAGgcaggca (SEQ ID NO: 105), AAGgcaggga (SEQ ID NO: 106), AAGgcagggg (SEQ ID NO: 107), AAGgcaggua (SEQ ID NO: 108), AAGgcaggug (SEQ ID NO: 109), AAGgcaucuc (SEQ ID NO: 110), AAGgcaugcu (SEQ ID NO: 111), AAGgcaugga (SEQ ID NO: 112), AAGgcauguu (SEQ ID NO: 113), AAGgcauuau (SEQ ID NO: 114), AAGgcgagcu (SEQ ID NO: 115), AAGgcgaguc (SEQ ID NO: 116), AAGgcgaguu (SEQ ID NO: 117), AAGgcuagcc (SEQ ID NO: 118), AAGguaaaaa (SEQ ID NO: 119), AAGguaaaac (SEQ ID NO: 120), AAGguaaaag (SEQ ID NO: 121), AAGguaaaau (SEQ ID NO: 122), AAGguaaaca (SEQ ID NO: 123), AAGguaaacc (SEQ ID NO: 124), AAGguaaacu (SEQ ID NO: 125), AAGguaaaga (SEQ ID NO: 126), AAGguaaagc (SEQ ID NO: 127), AAGguaaagg (SEQ ID NO: 128), AAGguaaagu (SEQ ID NO: 129), AAGguaaaua (SEQ ID NO: 130), AAGguaaauc (SEQ ID NO: 131), AAGguaaaug (SEQ ID NO: 132), AAGguaaauu (SEQ ID NO: 133), AAGguaacaa (SEQ ID NO: 134), AAGguaacau (SEQ ID NO: 135), AAGguaaccc (SEQ ID NO: 136), AAGguaacua (SEQ ID NO: 137), AAGguaacuc (SEQ ID NO: 138), AAGguaacug (SEQ ID NO: 139), AAGguaacuu (SEQ ID NO: 140), AAGguaagaa (SEQ ID NO: 141), AAGguaagac (SEQ ID NO: 142), AAGguaagag (SEQ ID NO: 143), AAGguaagau (SEQ ID NO: 144), AAGguaagca (SEQ ID NO: 145), AAGguaagcc (SEQ ID NO: 146), AAGguaagcg (SEQ ID NO: 147), AAGguaagcu (SEQ ID NO: 148), AAGguaagga (SEQ ID NO: 149), AAGguaaggc (SEQ ID NO: 150), AAGguaaggg (SEQ ID NO: 151), AAGguaaggu (SEQ ID NO: 152), AAGguaagua (SEQ ID NO: 153), AAGguaaguc (SEQ ID NO: 154), AAGguaagug (SEQ ID NO: 155), AAGguaaguu (SEQ ID NO: 156), AAGguaauaa (SEQ ID NO: 157), AAGguaauac (SEQ ID NO: 158), AAGguaauag (SEQ ID NO: 159), AAGguaauau (SEQ ID NO: 160), AAGguaauca (SEQ ID NO: 161), AAGguaaucc (SEQ ID NO: 162), AAGguaaucu (SEQ ID NO: 163), AAGguaauga (SEQ ID NO: 164), AAGguaaugc (SEQ ID NO: 165), AAGguaaugg (SEQ ID NO: 166), AAGguaaugu (SEQ ID NO: 167), AAGguaauua (SEQ ID NO: 168), AAGguaauuc (SEQ ID NO: 169), AAGguaauug (SEQ ID NO: 170), AAGguaauuu (SEQ ID NO: 171), AAGguacaaa (SEQ ID NO: 172), AAGguacaag (SEQ ID NO: 173), AAGguacaau (SEQ ID NO: 174), AAGguacacc (SEQ ID NO: 175), AAGguacacu (SEQ ID NO: 176), AAGguacagg (SEQ ID NO: 177), AAGguacagu (SEQ ID NO: 178), AAGguacaua (SEQ ID NO: 179), AAGguacaug (SEQ ID NO: 180), AAGguacauu (SEQ ID NO: 181), AAGguaccaa (SEQ ID NO: 182), AAGguaccag (SEQ ID NO: 183), AAGguaccca (SEQ ID NO: 184), AAGguacccu (SEQ ID NO: 185), AAGguaccuc (SEQ ID NO: 186), AAGguaccug (SEQ ID NO: 187), AAGguaccuu (SEQ ID NO: 188), AAGguacgaa (SEQ ID NO: 189), AAGguacggg (SEQ ID NO: 190), AAGguacggu (SEQ ID NO: 191), AAGguacguc (SEQ ID NO: 192), AAGguacguu (SEQ ID NO: 193), AAGguacuaa (SEQ ID NO: 194), AAGguacuau (SEQ ID NO: 195), AAGguacucu (SEQ ID NO: 196), AAGguacuga (SEQ ID NO: 197), AAGguacugc (SEQ ID NO: 198), AAGguacugu (SEQ ID NO: 199), AAGguacuuc (SEQ ID NO: 200), AAGguacuug (SEQ ID NO: 201), AAGguacuuu (SEQ ID NO: 202), AAGguagaaa (SEQ ID NO: 203), AAGguagaac (SEQ ID NO: 204), AAGguagaca (SEQ ID NO: 205), AAGguagacc (SEQ ID NO: 206), AAGguagacu (SEQ ID NO: 207), AAGguagagu (SEQ ID NO: 208), AAGguagaua (SEQ ID NO: 209), AAGguagcaa (SEQ ID NO: 210), AAGguagcag (SEQ ID NO: 211), AAGguagcca (SEQ ID NO: 212), AAGguagccu (SEQ ID NO: 213), AAGguagcua (SEQ ID NO: 214), AAGguagcug (SEQ ID NO: 215), AAGguagcuu (SEQ ID NO: 216), AAGguaggaa (SEQ ID NO: 217), AAGguaggag (SEQ ID NO: 218), AAGguaggau (SEQ ID NO: 219), AAGguaggca (SEQ ID NO: 220), AAGguaggcc (SEQ ID NO: 221), AAGguaggcu (SEQ ID NO: 222), AAGguaggga (SEQ ID NO: 223), AAGguagggc (SEQ ID NO: 224), AAGguagggg (SEQ ID NO: 225), AAGguagggu (SEQ ID NO: 226), AAGguaggua (SEQ ID NO: 227), AAGguagguc (SEQ ID NO: 228), AAGguaggug (SEQ ID NO: 229), AAGguagguu (SEQ ID NO: 230), AAGguaguaa (SEQ ID NO: 231), AAGguaguag (SEQ ID NO: 232), AAGguagucu (SEQ ID NO: 233), AAGguagugc (SEQ ID NO: 234), AAGguagugg (SEQ ID NO: 235), AAGguaguuc (SEQ ID NO: 236), AAGguaguuu (SEQ ID NO: 237), AAGguauaaa (SEQ ID NO: 238), AAGguauaau (SEQ ID NO: 239), AAGguauaca (SEQ ID NO: 240), AAGguauacu (SEQ ID NO: 241), AAGguauaua (SEQ ID NO: 242), AAGguauauc (SEQ ID NO: 243), AAGguauaug (SEQ ID NO: 244), AAGguauauu (SEQ ID NO: 245), AAGguaucac (SEQ ID NO: 246), AAGguaucag (SEQ ID NO: 247), AAGguauccc (SEQ ID NO: 248), AAGguauccu (SEQ ID NO: 249), AAGguaucuc (SEQ ID NO: 250), AAGguaucug (SEQ ID NO: 251), AAGguaucuu (SEQ ID NO: 252), AAGguaugaa (SEQ ID NO: 253), AAGguaugac (SEQ ID NO: 254), AAGguaugag (SEQ ID NO: 255), AAGguaugau (SEQ ID NO: 256), AAGguaugca (SEQ ID NO: 257), AAGguaugcc (SEQ ID NO: 258), AAGguaugcu (SEQ ID NO: 259), AAGguaugga (SEQ ID NO: 260), AAGguauggc (SEQ ID NO: 261), AAGguauggg (SEQ ID NO: 262), AAGguaugua (SEQ ID NO: 263), AAGguauguc (SEQ ID NO: 264), AAGguaugug (SEQ ID NO: 265), AAGguauguu (SEQ ID NO: 266), AAGguauuaa (SEQ ID NO: 267), AAGguauuac (SEQ ID NO: 268), AAGguauuag (SEQ ID NO: 269), AAGguauuau (SEQ ID NO: 270), AAGguauucc (SEQ ID NO: 271), AAGguauuga (SEQ ID NO: 272), AAGguauugu (SEQ ID NO: 273), AAGguauuua (SEQ ID NO: 274), AAGguauuuc (SEQ ID NO: 275), AAGguauuug (SEQ ID NO: 276), AAGguauuuu (SEQ ID NO: 277), AAGgucaaau (SEQ ID NO: 278), AAGgucaaga (SEQ ID NO: 279), AAGgucaagu (SEQ ID NO: 280), AAGgucacag (SEQ ID NO: 281), AAGgucagaa (SEQ ID NO: 282), AAGgucagac (SEQ ID NO: 283), AAGgucagag (SEQ ID NO: 284), AAGgucagca (SEQ ID NO: 285), AAGgucagcc (SEQ ID NO: 286), AAGgucagcg (SEQ ID NO: 287), AAGgucagcu (SEQ ID NO: 288), AAGgucagga (SEQ ID NO: 289), AAGgucaggc (SEQ ID NO: 290), AAGgucaggg (SEQ ID NO: 291), AAGgucaggu (SEQ ID NO: 292), AAGgucagua (SEQ ID NO: 293), AAGgucaguc (SEQ ID NO: 294), AAGgucagug (SEQ ID NO: 295), AAGgucaguu (SEQ ID NO: 296), AAGgucauag (SEQ ID NO: 297), AAGgucaucu (SEQ ID NO: 298), AAGguccaca (SEQ ID NO: 299), AAGguccaga (SEQ ID NO: 300), AAGguccaua (SEQ ID NO: 301), AAGgucccag (SEQ ID NO: 302), AAGgucccuc (SEQ ID NO: 303), AAGguccuuc (SEQ ID NO: 304), AAGgucgagg (SEQ ID NO: 305), AAGgucuaau (SEQ ID NO: 306), AAGgucuacc (SEQ ID NO: 307), AAGgucuaua (SEQ ID NO: 308), AAGgucuccu (SEQ ID NO: 309), AAGgucucug (SEQ ID NO: 310), AAGgucucuu (SEQ ID NO: 311), AAGgucugaa (SEQ ID NO: 312), AAGgucugag (SEQ ID NO: 313), AAGgucugga (SEQ ID NO: 314), AAGgucuggg (SEQ ID NO: 315), AAGgucugua (SEQ ID NO: 316), AAGgucuguu (SEQ ID NO: 317), AAGgucuucu (SEQ ID NO: 318), AAGgucuuuu (SEQ ID NO: 319), AAGgugaaac (SEQ ID NO: 320), AAGgugaaag (SEQ ID NO: 321), AAGgugaaau (SEQ ID NO: 322), AAGgugaacu (SEQ ID NO: 323), AAGgugaagc (SEQ ID NO: 324), AAGgugaagg (SEQ ID NO: 325), AAGgugaagu (SEQ ID NO: 326), AAGgugaaua (SEQ ID NO: 327), AAGgugaaug (SEQ ID NO: 328), AAGgugaauu (SEQ ID NO: 329), AAGgugacaa (SEQ ID NO: 330), AAGgugacag (SEQ ID NO: 331), AAGgugacau (SEQ ID NO: 332), AAGgugacug (SEQ ID NO: 333), AAGgugacuu (SEQ ID NO: 334), AAGgugagaa (SEQ ID NO: 335), AAGgugagac (SEQ ID NO: 336), AAGgugagag (SEQ ID NO: 337), AAGgugagau (SEQ ID NO: 338), AAGgugagca (SEQ ID NO: 339), AAGgugagcc (SEQ ID NO: 340), AAGgugagcg (SEQ ID NO: 341), AAGgugagcu (SEQ ID NO: 342), AAGgugagga (SEQ ID NO: 343), AAGgugaggc (SEQ ID NO: 344), AAGgugaggg (SEQ ID NO: 345), AAGgugaggu (SEQ ID NO: 346), AAGgugagua (SEQ ID NO: 347), AAGgugaguc (SEQ ID NO: 348), AAGgugagug (SEQ ID NO: 349), AAGgugaguu (SEQ ID NO: 350), AAGgugauaa (SEQ ID NO: 351), AAGgugauca (SEQ ID NO: 352), AAGgugaucc (SEQ ID NO: 353), AAGgugauga (SEQ ID NO: 354), AAGgugaugc (SEQ ID NO: 355), AAGgugaugu (SEQ ID NO: 356), AAGgugauua (SEQ ID NO: 357), AAGgugauug (SEQ ID NO: 358), AAGgugauuu (SEQ ID NO: 359), AAGgugcaca (SEQ ID NO: 360), AAGgugcauc (SEQ ID NO: 361), AAGgugcccu (SEQ ID NO: 362), AAGgugccug (SEQ ID NO: 363), AAGgugcgug (SEQ ID NO: 364), AAGgugcguu (SEQ ID NO: 365), AAGgugcucc (SEQ ID NO: 366), AAGgugcuga (SEQ ID NO: 367), AAGgugcugc (SEQ ID NO: 368), AAGgugcugg (SEQ ID NO: 369), AAGgugcuua (SEQ ID NO: 370), AAGgugcuuu (SEQ ID NO: 371), AAGguggaua (SEQ ID NO: 372), AAGguggcua (SEQ ID NO: 373), AAGguggcug (SEQ ID NO: 374), AAGguggcuu (SEQ ID NO: 375), AAGgugggaa (SEQ ID NO: 376), AAGgugggag (SEQ ID NO: 377), AAGgugggau (SEQ ID NO: 378), AAGgugggca (SEQ ID NO: 379), AAGgugggcc (SEQ ID NO: 380), AAGgugggcg (SEQ ID NO: 381), AAGgugggga (SEQ ID NO: 382), AAGguggggu (SEQ ID NO: 383), AAGgugggua (SEQ ID NO: 384), AAGgugggug (SEQ ID NO: 385), AAGguggguu (SEQ ID NO: 386), AAGgugguaa (SEQ ID NO: 387), AAGgugguac (SEQ ID NO: 388), AAGgugguau (SEQ ID NO: 389), AAGguggugg (SEQ ID NO: 390), AAGgugguua (SEQ ID NO: 391), AAGgugguuc (SEQ ID NO: 392), AAGgugguuu (SEQ ID NO: 393), AAGguguaag (SEQ ID NO: 394), AAGgugucaa (SEQ ID NO: 395), AAGgugucag (SEQ ID NO: 396), AAGgugucug (SEQ ID NO: 397), AAGgugugaa (SEQ ID NO: 398), AAGgugugag (SEQ ID NO: 399), AAGgugugca (SEQ ID NO: 400), AAGgugugga (SEQ ID NO: 401), AAGguguggu (SEQ ID NO: 402), AAGgugugua (SEQ ID NO: 403), AAGguguguc (SEQ ID NO: 404), AAGgugugug (SEQ ID NO: 405), AAGguguguu (SEQ ID NO: 406), AAGguguucu (SEQ ID NO: 407), AAGguguugc (SEQ ID NO: 408), AAGguguugg (SEQ ID NO: 409), AAGguguuug (SEQ ID NO: 410), AAGguuaaaa (SEQ ID NO: 411), AAGguuaaca (SEQ ID NO: 412), AAGguuaagc (SEQ ID NO: 413), AAGguuaauu (SEQ ID NO: 414), AAGguuacau (SEQ ID NO: 415), AAGguuagaa (SEQ ID NO: 416), AAGguuagau (SEQ ID NO: 417), AAGguuagca (SEQ ID NO: 418), AAGguuagcc (SEQ ID NO: 419), AAGguuagga (SEQ ID NO: 420), AAGguuaggc (SEQ ID NO: 421), AAGguuagua (SEQ ID NO: 422), AAGguuaguc (SEQ ID NO: 423), AAGguuagug (SEQ ID NO: 424), AAGguuaguu (SEQ ID NO: 425), AAGguuauag (SEQ ID NO: 426), AAGguuauga (SEQ ID NO: 427), AAGguucaaa (SEQ ID NO: 428), AAGguucaag (SEQ ID NO: 429), AAGguuccuu (SEQ ID NO: 430), AAGguucggc (SEQ ID NO: 431), AAGguucguu (SEQ ID NO: 432), AAGguucuaa (SEQ ID NO: 433), AAGguucuga (SEQ ID NO: 434), AAGguucuua (SEQ ID NO: 435), AAGguugaau (SEQ ID NO: 436), AAGguugacu (SEQ ID NO: 437), AAGguugagg (SEQ ID NO: 438), AAGguugagu (SEQ ID NO: 439), AAGguugaua (SEQ ID NO: 440), AAGguugcac (SEQ ID NO: 441), AAGguugcug (SEQ ID NO: 442), AAGguuggaa (SEQ ID NO: 443), AAGguuggca (SEQ ID NO: 444), AAGguuggga (SEQ ID NO: 445), AAGguugggg (SEQ ID NO: 446), AAGguuggua (SEQ ID NO: 447), AAGguugguc (SEQ ID NO: 448), AAGguuggug (SEQ ID NO: 449), AAGguugguu (SEQ ID NO: 450), AAGguuguaa (SEQ ID NO: 451), AAGguugucc (SEQ ID NO: 452), AAGguugugc (SEQ ID NO: 453), AAGguuguua (SEQ ID NO: 454), AAGguuuacc (SEQ ID NO: 455), AAGguuuaua (SEQ ID NO: 456), AAGguuuauu (SEQ ID NO: 457), AAGguuuccu (SEQ ID NO: 458), AAGguuucgu (SEQ ID NO: 459), AAGguuugag (SEQ ID NO: 460), AAGguuugca (SEQ ID NO: 461), AAGguuugcc (SEQ ID NO: 462), AAGguuugcu (SEQ ID NO: 463), AAGguuugga (SEQ ID NO: 464), AAGguuuggu (SEQ ID NO: 465), AAGguuugua (SEQ ID NO: 466), AAGguuuguc (SEQ ID NO: 467), AAGguuugug (SEQ ID NO: 468), AAGguuuuaa (SEQ ID NO: 469), AAGguuuuca (SEQ ID NO: 470), AAGguuuucg (SEQ ID NO: 471), AAGguuuugc (SEQ ID NO: 472), AAGguuuugu (SEQ ID NO: 473), AAGguuuuuu (SEQ ID NO: 474), AAUgcaagua (SEQ ID NO: 475), AAUgcaaguc (SEQ ID NO: 476), AAUguaaaca (SEQ ID NO: 477), AAUguaaaua (SEQ ID NO: 478), AAUguaaauc (SEQ ID NO: 479), AAUguaaaug (SEQ ID NO: 480), AAUguaaauu (SEQ ID NO: 481), AAUguaacua (SEQ ID NO: 482), AAUguaagaa (SEQ ID NO: 483), AAUguaagag (SEQ ID NO: 484), AAUguaagau (SEQ ID NO: 485), AAUguaagcc (SEQ ID NO: 486), AAUguaagcu (SEQ ID NO: 487), AAUguaagga (SEQ ID NO: 488), AAUguaagua (SEQ ID NO: 489), AAUguaaguc (SEQ ID NO: 490), AAUguaagug (SEQ ID NO: 491), AAUguaaguu (SEQ ID NO: 492), AAUguaauca (SEQ ID NO: 493), AAUguaauga (SEQ ID NO: 494), AAUguaaugu (SEQ ID NO: 495), AAUguacauc (SEQ ID NO: 496), AAUguacaug (SEQ ID NO: 497), AAUguacgau (SEQ ID NO: 498), AAUguacgua (SEQ ID NO: 499), AAUguacguc (SEQ ID NO: 500), AAUguacgug (SEQ ID NO: 501), AAUguacucu (SEQ ID NO: 502), AAUguaggca (SEQ ID NO: 503), AAUguagguu (SEQ ID NO: 504), AAUguaucua (SEQ ID NO: 505), AAUguaugaa (SEQ ID NO: 506), AAUguaugua (SEQ ID NO: 507), AAUguaugug (SEQ ID NO: 508), AAUguauguu (SEQ ID NO: 509), AAUgucagag (SEQ ID NO: 510), AAUgucagau (SEQ ID NO: 511), AAUgucagcu (SEQ ID NO: 512), AAUgucagua (SEQ ID NO: 513), AAUgucaguc (SEQ ID NO: 514), AAUgucagug (SEQ ID NO: 515), AAUgucaguu (SEQ ID NO: 516), AAUgucggua (SEQ ID NO: 517), AAUgucuguu (SEQ ID NO: 518), AAUgugagaa (SEQ ID NO: 519), AAUgugagca (SEQ ID NO: 520), AAUgugagcc (SEQ ID NO: 521), AAUgugagga (SEQ ID NO: 522), AAUgugagua (SEQ ID NO: 523), AAUgugaguc (SEQ ID NO: 524), AAUgugagug (SEQ ID NO: 525), AAUgugaguu (SEQ ID NO: 526), AAUgugauau (SEQ ID NO: 527), AAUgugcaua (SEQ ID NO: 528), AAUgugcgua (SEQ ID NO: 529), AAUgugcguc (SEQ ID NO: 530), AAUgugggac (SEQ ID NO: 531), AAUguggguc (SEQ ID NO: 532), AAUgugggug (SEQ ID NO: 533), AAUgugguuu (SEQ ID NO: 534), AAUgugugua (SEQ ID NO: 535), AAUguuaagu (SEQ ID NO: 536), AAUguuagaa (SEQ ID NO: 537), AAUguuagau (SEQ ID NO: 538), AAUguuagua (SEQ ID NO: 539), AAUguuggug (SEQ ID NO: 540), ACAgcaagua (SEQ ID NO: 541), ACAguaaaua (SEQ ID NO: 542), ACAguaaaug (SEQ ID NO: 543), ACAguaagaa (SEQ ID NO: 544), ACAguaagca (SEQ ID NO: 545), ACAguaagua (SEQ ID NO: 546), ACAguaaguc (SEQ ID NO: 547), ACAguaagug (SEQ ID NO: 548), ACAguaaguu (SEQ ID NO: 549), ACAguacgua (SEQ ID NO: 550), ACAguaggug (SEQ ID NO: 551), ACAguauaac (SEQ ID NO: 552), ACAguaugua (SEQ ID NO: 553), ACAgucaguu (SEQ ID NO: 554), ACAgugagaa (SEQ ID NO: 555), ACAgugagcc (SEQ ID NO: 556), ACAgugagcu (SEQ ID NO: 557), ACAgugagga (SEQ ID NO: 558), ACAgugaggu (SEQ ID NO: 559), ACAgugagua (SEQ ID NO: 560), ACAgugaguc (SEQ ID NO: 561), ACAgugagug (SEQ ID NO: 562), ACAgugaguu (SEQ ID NO: 563), ACAgugggua (SEQ ID NO: 564), ACAguggguu (SEQ ID NO: 565), ACAguguaaa (SEQ ID NO: 566), ACAguuaagc (SEQ ID NO: 567), ACAguuaagu (SEQ ID NO: 568), ACAguuaugu (SEQ ID NO: 569), ACAguugagu (SEQ ID NO: 570), ACAguuguga (SEQ ID NO: 571), ACCguaagua (SEQ ID NO: 572), ACCgugagaa (SEQ ID NO: 573), ACCgugagca (SEQ ID NO: 574), ACCgugaguu (SEQ ID NO: 575), ACCgugggug (SEQ ID NO: 576), ACGguaaaac (SEQ ID NO: 577), ACGguaacua (SEQ ID NO: 578), ACGguaagua (SEQ ID NO: 579), ACGguaagug (SEQ ID NO: 580), ACGguaaguu (SEQ ID NO: 581), ACGguaauua (SEQ ID NO: 582), ACGguaauuu (SEQ ID NO: 583), ACGguacaau (SEQ ID NO: 584), ACGguacagu (SEQ ID NO: 585), ACGguaccag (SEQ ID NO: 586), ACGguacggu (SEQ ID NO: 587), ACGguacgua (SEQ ID NO: 588), ACGguaggaa (SEQ ID NO: 589), ACGguaggag (SEQ ID NO: 590), ACGguaggug (SEQ ID NO: 591), ACGguaguaa (SEQ ID NO: 592), ACGguauaau (SEQ ID NO: 593), ACGguaugac (SEQ ID NO: 594), ACGguaugcg (SEQ ID NO: 595), ACGguaugua (SEQ ID NO: 596), ACGguauguc (SEQ ID NO: 597), ACGgugaaac (SEQ ID NO: 598), ACGgugaagu (SEQ ID NO: 599), ACGgugaauc (SEQ ID NO: 600), ACGgugacag (SEQ ID NO: 601), ACGgugacca (SEQ ID NO: 602), ACGgugagaa (SEQ ID NO: 603), ACGgugagau (SEQ ID NO: 604), ACGgugagcc (SEQ ID NO: 605), ACGgugagua (SEQ ID NO: 606), ACGgugagug (SEQ ID NO: 607), ACGgugaguu (SEQ ID NO: 608), ACGgugcgug (SEQ ID NO: 609), ACGguggcac (SEQ ID NO: 610), ACGguggggc (SEQ ID NO: 611), ACGgugggug (SEQ ID NO: 612), ACGguguagu (SEQ ID NO: 613), ACGgugucac (SEQ ID NO: 614), ACGgugugua (SEQ ID NO: 615), ACGguguguu (SEQ ID NO: 616), ACGguuagug (SEQ ID NO: 617), ACGguuaguu (SEQ ID NO: 618), ACGguucaau (SEQ ID NO: 619), ACUguaaaua (SEQ ID NO: 620), ACUguaagaa (SEQ ID NO: 621), ACUguaagac (SEQ ID NO: 622), ACUguaagca (SEQ ID NO: 623), ACUguaagcu (SEQ ID NO: 624), ACUguaagua (SEQ ID NO: 625), ACUguaaguc (SEQ ID NO: 626), ACUguaaguu (SEQ ID NO: 627), ACUguacguu (SEQ ID NO: 628), ACUguacuge (SEQ ID NO: 629), ACUguaggcu (SEQ ID NO: 630), ACUguaggua (SEQ ID NO: 631), ACUguauauu (SEQ ID NO: 632), ACUguaugaa (SEQ ID NO: 633), ACUguaugcu (SEQ ID NO: 634), ACUguaugug (SEQ ID NO: 635), ACUguauucc (SEQ ID NO: 636), ACUgucagcu (SEQ ID NO: 637), ACUgucagug (SEQ ID NO: 638), ACUgugaacg (SEQ ID NO: 639), ACUgugagca (SEQ ID NO: 640), ACUgugagcg (SEQ ID NO: 641), ACUgugagcu (SEQ ID NO: 642), ACUgugagua (SEQ ID NO: 643), ACUgugaguc (SEQ ID NO: 644), ACUgugagug (SEQ ID NO: 645), ACUgugaguu (SEQ ID NO: 646), ACUgugggua (SEQ ID NO: 647), ACUgugugug (SEQ ID NO: 648), ACUguuaagu (SEQ ID NO: 649), AGAgcaagua (SEQ ID NO: 650), AGAguaaaac (SEQ ID NO: 651), AGAguaaacg (SEQ ID NO: 652), AGAguaaaga (SEQ ID NO: 653), AGAguaaagu (SEQ ID NO: 654), AGAguaaauc (SEQ ID NO: 655), AGAguaaaug (SEQ ID NO: 656), AGAguaacau (SEQ ID NO: 657), AGAguaacua (SEQ ID NO: 658), AGAguaagaa (SEQ ID NO: 659), AGAguaagac (SEQ ID NO: 660), AGAguaagag (SEQ ID NO: 661), AGAguaagau (SEQ ID NO: 662), AGAguaagca (SEQ ID NO: 663), AGAguaagcu (SEQ ID NO: 664), AGAguaagga (SEQ ID NO: 665), AGAguaaggc (SEQ ID NO: 666), AGAguaaggg (SEQ ID NO: 667), AGAguaaggu (SEQ ID NO: 668), AGAguaaguc (SEQ ID NO: 669), AGAguaagug (SEQ ID NO: 670), AGAguaaguu (SEQ ID NO: 671), AGAguaauaa (SEQ ID NO: 672), AGAguaaugu (SEQ ID NO: 673), AGAguaauuc (SEQ ID NO: 674), AGAguaauuu (SEQ ID NO: 675), AGAguacacc (SEQ ID NO: 676), AGAguaccug (SEQ ID NO: 677), AGAguacgug (SEQ ID NO: 678), AGAguacucu (SEQ ID NO: 679), AGAguacuga (SEQ ID NO: 680), AGAguacuuu (SEQ ID NO: 681), AGAguagcug (SEQ ID NO: 682), AGAguaggaa (SEQ ID NO: 683), AGAguaggga (SEQ ID NO: 684), AGAguagggu (SEQ ID NO: 685), AGAguagguc (SEQ ID NO: 686), AGAguaggug (SEQ ID NO: 687), AGAguagguu (SEQ ID NO: 688), AGAguauaua (SEQ ID NO: 689), AGAguauauu (SEQ ID NO: 690), AGAguaugaa (SEQ ID NO: 691), AGAguaugac (SEQ ID NO: 692), AGAguaugau (SEQ ID NO: 693), AGAguauguc (SEQ ID NO: 694), AGAguaugug (SEQ ID NO: 695), AGAguauguu (SEQ ID NO: 696), AGAguauuaa (SEQ ID NO: 697), AGAguauuau (SEQ ID NO: 698), AGAgucagug (SEQ ID NO: 699), AGAgugagac (SEQ ID NO: 700), AGAgugagag (SEQ ID NO: 701), AGAgugagau (SEQ ID NO: 702), AGAgugagca (SEQ ID NO: 703), AGAgugagua (SEQ ID NO: 704), AGAgugaguc (SEQ ID NO: 705), AGAgugagug (SEQ ID NO: 706), AGAgugaguu (SEQ ID NO: 707), AGAgugcguc (SEQ ID NO: 708), AGAgugggga (SEQ ID NO: 709), AGAgugggug (SEQ ID NO: 710), AGAgugugug (SEQ ID NO: 711), AGAguguuuc (SEQ ID NO: 712), AGAguuagua (SEQ ID NO: 713), AGAguugaga (SEQ ID NO: 714), AGAguugagu (SEQ ID NO: 715), AGAguugguu (SEQ ID NO: 716), AGAguuugau (SEQ ID NO: 717), AGCguaagcu (SEQ ID NO: 718), AGCguaagug (SEQ ID NO: 719), AGCgugagcc (SEQ ID NO: 720), AGCgugagug (SEQ ID NO: 721), AGCguuguuc (SEQ ID NO: 722), AGGgcagagu (SEQ ID NO: 723), AGGgcagccu (SEQ ID NO: 724), AGGgcuagua (SEQ ID NO: 725), AGGguaaaga (SEQ ID NO: 726), AGGguaaaua (SEQ ID NO: 727), AGGguaaauc (SEQ ID NO: 728), AGGguaaauu (SEQ ID NO: 729), AGGguaacca (SEQ ID NO: 730), AGGguaacug (SEQ ID NO: 731), AGGguaacuu (SEQ ID NO: 732), AGGguaagaa (SEQ ID NO: 733), AGGguaagag (SEQ ID NO: 734), AGGguaagau (SEQ ID NO: 735), AGGguaagca (SEQ ID NO: 736), AGGguaagga (SEQ ID NO: 737), AGGguaaggc (SEQ ID NO: 738), AGGguaaggg (SEQ ID NO: 739), AGGguaagua (SEQ ID NO: 740), AGGguaaguc (SEQ ID NO: 741), AGGguaagug (SEQ ID NO: 742), AGGguaaguu (SEQ ID NO: 743), AGGguaauac (SEQ ID NO: 744), AGGguaauga (SEQ ID NO: 745), AGGguaauua (SEQ ID NO: 746), AGGguaauuu (SEQ ID NO: 747), AGGguacacc (SEQ ID NO: 748), AGGguacagu (SEQ ID NO: 749), AGGguacggu (SEQ ID NO: 750), AGGguaggac (SEQ ID NO: 751), AGGguaggag (SEQ ID NO: 752), AGGguaggca (SEQ ID NO: 753), AGGguaggcc (SEQ ID NO: 754), AGGguaggga (SEQ ID NO: 755), AGGguagggu (SEQ ID NO: 756), AGGguagguc (SEQ ID NO: 757), AGGguaggug (SEQ ID NO: 758), AGGguagguu (SEQ ID NO: 759), AGGguauaua (SEQ ID NO: 760), AGGguaugac (SEQ ID NO: 761), AGGguaugag (SEQ ID NO: 762), AGGguaugau (SEQ ID NO: 763), AGGguaugca (SEQ ID NO: 764), AGGguaugcu (SEQ ID NO: 765), AGGguauggg (SEQ ID NO: 766), AGGguauggu (SEQ ID NO: 767), AGGguaugua (SEQ ID NO: 768), AGGguauguc (SEQ ID NO: 769), AGGguaugug (SEQ ID NO: 770), AGGguauuac (SEQ ID NO: 771), AGGguauucu (SEQ ID NO: 772), AGGguauuuc (SEQ ID NO: 773), AGGgucagag (SEQ ID NO: 774), AGGgucagca (SEQ ID NO: 775), AGGgucagga (SEQ ID NO: 776), AGGgucaggg (SEQ ID NO: 777), AGGgucagug (SEQ ID NO: 778), AGGgucaguu (SEQ ID NO: 779), AGGguccccu (SEQ ID NO: 780), AGGgucggga (SEQ ID NO: 781), AGGgucugca (SEQ ID NO: 782), AGGgucuguu (SEQ ID NO: 783), AGGgugaaga (SEQ ID NO: 784), AGGgugacua (SEQ ID NO: 785), AGGgugagaa (SEQ ID NO: 786), AGGgugagac (SEQ ID NO: 787), AGGgugagag (SEQ ID NO: 788), AGGgugagca (SEQ ID NO: 789), AGGgugagcc (SEQ ID NO: 790), AGGgugagcu (SEQ ID NO: 791), AGGgugagga (SEQ ID NO: 792), AGGgugaggg (SEQ ID NO: 793), AGGgugaggu (SEQ ID NO: 794), AGGgugagua (SEQ ID NO: 795), AGGgugaguc (SEQ ID NO: 796), AGGgugagug (SEQ ID NO: 797), AGGgugaguu (SEQ ID NO: 798), AGGgugggga (SEQ ID NO: 799), AGGguggggu (SEQ ID NO: 800), AGGgugggua (SEQ ID NO: 801), AGGgugggug (SEQ ID NO: 802), AGGgugugua (SEQ ID NO: 803), AGGgugugug (SEQ ID NO: 804), AGGguuaaug (SEQ ID NO: 805), AGGguuagaa (SEQ ID NO: 806), AGGguuaguu (SEQ ID NO: 807), AGGguuggug (SEQ ID NO: 808), AGGguuugug (SEQ ID NO: 809), AGGguuuguu (SEQ ID NO: 810), AGUguaaaag (SEQ ID NO: 811), AGUguaaaua (SEQ ID NO: 812), AGUguaaauu (SEQ ID NO: 813), AGUguaagaa (SEQ ID NO: 814), AGUguaagag (SEQ ID NO: 815), AGUguaagau (SEQ ID NO: 816), AGUguaagca (SEQ ID NO: 817), AGUguaagcc (SEQ ID NO: 818), AGUguaagua (SEQ ID NO: 819), AGUguaagug (SEQ ID NO: 820), AGUguaaguu (SEQ ID NO: 821), AGUguaauug (SEQ ID NO: 822), AGUguaggac (SEQ ID NO: 823), AGUguagguc (SEQ ID NO: 824), AGUguaugag (SEQ ID NO: 825), AGUguaugua (SEQ ID NO: 826), AGUguauguu (SEQ ID NO: 827), AGUguauugu (SEQ ID NO: 828), AGUguauuua (SEQ ID NO: 829), AGUgucaguc (SEQ ID NO: 830), AGUgugagag (SEQ ID NO: 831), AGUgugagca (SEQ ID NO: 832), AGUgugagcc (SEQ ID NO: 833), AGUgugagcu (SEQ ID NO: 834), AGUgugagua (SEQ ID NO: 835), AGUgugaguc (SEQ ID NO: 836), AGUgugagug (SEQ ID NO: 837), AGUgugaguu (SEQ ID NO: 838), AGUgugggua (SEQ ID NO: 839), AGUgugggug (SEQ ID NO: 840), AGUgugugua (SEQ ID NO: 841), AGUguuccua (SEQ ID NO: 842), AGUguugggg (SEQ ID NO: 843), AGUguuucag (SEQ ID NO: 844), AUAguaaaua (SEQ ID NO: 845), AUAguaagac (SEQ ID NO: 846), AUAguaagau (SEQ ID NO: 847), AUAguaagca (SEQ ID NO: 848), AUAguaagua (SEQ ID NO: 849), AUAguaagug (SEQ ID NO: 850), AUAguaaguu (SEQ ID NO: 851), AUAguaggua (SEQ ID NO: 852), AUAguauguu (SEQ ID NO: 853), AUAgucucac (SEQ ID NO: 854), AUAgugagac (SEQ ID NO: 855), AUAgugagag (SEQ ID NO: 856), AUAgugagau (SEQ ID NO: 857), AUAgugagcc (SEQ ID NO: 858), AUAgugaggc (SEQ ID NO: 859), AUAgugagua (SEQ ID NO: 860), AUAgugaguc (SEQ ID NO: 861), AUAgugagug (SEQ ID NO: 862), AUAgugcguc (SEQ ID NO: 863), AUAgugugua (SEQ ID NO: 864), AUAguucagu (SEQ ID NO: 865), AUCguaagcc (SEQ ID NO: 866), AUCguaaguu (SEQ ID NO: 867), AUCguauucc (SEQ ID NO: 868), AUCgugagua (SEQ ID NO: 869), AUGgcaagcg (SEQ ID NO: 870), AUGgcaagga (SEQ ID NO: 871), AUGgcaaguu (SEQ ID NO: 872), AUGgcaggua (SEQ ID NO: 873), AUGgcaugug (SEQ ID NO: 874), AUGgcgccau (SEQ ID NO: 875), AUGgcuugug (SEQ ID NO: 876), AUGguaaaac (SEQ ID NO: 877), AUGguaaaau (SEQ ID NO: 878), AUGguaaacc (SEQ ID NO: 879), AUGguaaaga (SEQ ID NO: 880), AUGguaaaua (SEQ ID NO: 881), AUGguaaaug (SEQ ID NO: 882), AUGguaaauu (SEQ ID NO: 883), AUGguaacag (SEQ ID NO: 884), AUGguaacau (SEQ ID NO: 885), AUGguaacua (SEQ ID NO: 886), AUGguaacuc (SEQ ID NO: 887), AUGguaacuu (SEQ ID NO: 888), AUGguaagaa (SEQ ID NO: 889), AUGguaagac (SEQ ID NO: 890), AUGguaagag (SEQ ID NO: 891), AUGguaagau (SEQ ID NO: 892), AUGguaagca (SEQ ID NO: 893), AUGguaagcc (SEQ ID NO: 894), AUGguaagcu (SEQ ID NO: 895), AUGguaagga (SEQ ID NO: 896), AUGguaaggg (SEQ ID NO: 897), AUGguaagua (SEQ ID NO: 898), AUGguaaguc (SEQ ID NO: 899), AUGguaagug (SEQ ID NO: 900), AUGguaaguu (SEQ ID NO: 901), AUGguaauaa (SEQ ID NO: 902), AUGguaauau (SEQ ID NO: 903), AUGguaauga (SEQ ID NO: 904), AUGguaaugg (SEQ ID NO: 905), AUGguaauug (SEQ ID NO: 906), AUGguaauuu (SEQ ID NO: 907), AUGguacagc (SEQ ID NO: 908), AUGguacauc (SEQ ID NO: 909), AUGguaccag (SEQ ID NO: 910), AUGguaccug (SEQ ID NO: 911), AUGguacgag (SEQ ID NO: 912), AUGguacggu (SEQ ID NO: 913), AUGguagauc (SEQ ID NO: 914), AUGguagcag (SEQ ID NO: 915), AUGguagcug (SEQ ID NO: 916), AUGguaggaa (SEQ ID NO: 917), AUGguaggau (SEQ ID NO: 918), AUGguaggca (SEQ ID NO: 919), AUGguaggcu (SEQ ID NO: 920), AUGguagggg (SEQ ID NO: 921), AUGguagggu (SEQ ID NO: 922), AUGguaggua (SEQ ID NO: 923), AUGguaggug (SEQ ID NO: 924), AUGguaguuu (SEQ ID NO: 925), AUGguauagu (SEQ ID NO: 926), AUGguauaua (SEQ ID NO: 927), AUGguaucag (SEQ ID NO: 928), AUGguaucuu (SEQ ID NO: 929), AUGguaugau (SEQ ID NO: 930), AUGguaugca (SEQ ID NO: 931), AUGguaugcc (SEQ ID NO: 932), AUGguaugcg (SEQ ID NO: 933), AUGguaugcu (SEQ ID NO: 934), AUGguaugga (SEQ ID NO: 935), AUGguauggc (SEQ ID NO: 936), AUGguaugug (SEQ ID NO: 937), AUGguauguu (SEQ ID NO: 938), AUGguauuau (SEQ ID NO: 939), AUGguauuga (SEQ ID NO: 940), AUGguauuug (SEQ ID NO: 941), AUGgucaggg (SEQ ID NO: 942), AUGgucaguc (SEQ ID NO: 943), AUGgucagug (SEQ ID NO: 944), AUGgucauuu (SEQ ID NO: 945), AUGgugaaaa (SEQ ID NO: 946), AUGgugaaac (SEQ ID NO: 947), AUGgugaaau (SEQ ID NO: 948), AUGgugaacu (SEQ ID NO: 949), AUGgugaaga (SEQ ID NO: 950), AUGgugacgu (SEQ ID NO: 951), AUGgugagaa (SEQ ID NO: 952), AUGgugagac (SEQ ID NO: 953), AUGgugagag (SEQ ID NO: 954), AUGgugagca (SEQ ID NO: 955), AUGgugagcc (SEQ ID NO: 956), AUGgugagcg (SEQ ID NO: 957), AUGgugagcu (SEQ ID NO: 958), AUGgugaggc (SEQ ID NO: 959), AUGgugaggg (SEQ ID NO: 960), AUGgugagua (SEQ ID NO: 961), AUGgugaguc (SEQ ID NO: 962), AUGgugagug (SEQ ID NO: 963), AUGgugaguu (SEQ ID NO: 964), AUGgugauuu (SEQ ID NO: 965), AUGgugcgau (SEQ ID NO: 966), AUGgugcgug (SEQ ID NO: 967), AUGgugggua (SEQ ID NO: 968), AUGgugggug (SEQ ID NO: 969), AUGguggguu (SEQ ID NO: 970), AUGgugguua (SEQ ID NO: 971), AUGguguaag (SEQ ID NO: 972), AUGgugugaa (SEQ ID NO: 973), AUGgugugua (SEQ ID NO: 974), AUGgugugug (SEQ ID NO: 975), AUGguuacuc (SEQ ID NO: 976), AUGguuagca (SEQ ID NO: 977), AUGguuaguc (SEQ ID NO: 978), AUGguuagug (SEQ ID NO: 979), AUGguuaguu (SEQ ID NO: 980), AUGguucagu (SEQ ID NO: 981), AUGguucguc (SEQ ID NO: 982), AUGguuggua (SEQ ID NO: 983), AUGguugguc (SEQ ID NO: 984), AUGguugguu (SEQ ID NO: 985), AUGguuguuu (SEQ ID NO: 986), AUGguuugca (SEQ ID NO: 987), AUGguuugua (SEQ ID NO: 988), AUUgcaagua (SEQ ID NO: 989), AUUguaaaua (SEQ ID NO: 990), AUUguaagau (SEQ ID NO: 991), AUUguaagca (SEQ ID NO: 992), AUUguaagga (SEQ ID NO: 993), AUUguaaggc (SEQ ID NO: 994), AUUguaagua (SEQ ID NO: 995), AUUguaaguc (SEQ ID NO: 996), AUUguaaguu (SEQ ID NO: 997), AUUguaauua (SEQ ID NO: 998), AUUguaauuu (SEQ ID NO: 999), AUUguacaaa (SEQ ID NO: 1000), AUUguaccuc (SEQ ID NO: 1001), AUUguacgug (SEQ ID NO: 1002), AUUguacuug (SEQ ID NO: 1003), AUUguaggua (SEQ ID NO: 1004), AUUguaugag (SEQ ID NO: 1005), AUUguaugua (SEQ ID NO: 1006), AUUgucuguu (SEQ ID NO: 1007), AUUgugagcu (SEQ ID NO: 1008), AUUgugagua (SEQ ID NO: 1009), AUUgugaguc (SEQ ID NO: 1010), AUUgugaguu (SEQ ID NO: 1011), AUUgugcgug (SEQ ID NO: 1012), AUUgugggug (SEQ ID NO: 1013), AUUguuagug (SEQ ID NO: 1014), CAAguaaaaa (SEQ ID NO: 1015), CAAguaaaua (SEQ ID NO: 1016), CAAguaaauc (SEQ ID NO: 1017), CAAguaaaug (SEQ ID NO: 1018), CAAguaaccc (SEQ ID NO: 1019), CAAguaacua (SEQ ID NO: 1020), CAAguaacug (SEQ ID NO: 1021), CAAguaagaa (SEQ ID NO: 1022), CAAguaagac (SEQ ID NO: 1023), CAAguaagau (SEQ ID NO: 1024), CAAguaaggu (SEQ ID NO: 1025), CAAguaagua (SEQ ID NO: 1026), CAAguaaguc (SEQ ID NO: 1027), CAAguaagug (SEQ ID NO: 1028), CAAguaaguu (SEQ ID NO: 1029), CAAguaaucc (SEQ ID NO: 1030), CAAguaaucu (SEQ ID NO: 1031), CAAguaauua (SEQ ID NO: 1032), CAAguaauuc (SEQ ID NO: 1033), CAAguaauug (SEQ ID NO: 1034), CAAguaauuu (SEQ ID NO: 1035), CAAguacaca (SEQ ID NO: 1036), CAAguacguu (SEQ ID NO: 1037), CAAguacuuu (SEQ ID NO: 1038), CAAguagcug (SEQ ID NO: 1039), CAAguaggau (SEQ ID NO: 1040), CAAguaggua (SEQ ID NO: 1041), CAAguagguc (SEQ ID NO: 1042), CAAguaggug (SEQ ID NO: 1043), CAAguagguu (SEQ ID NO: 1044), CAAguaguuu (SEQ ID NO: 1045), CAAguauaac (SEQ ID NO: 1046), CAAguauaug (SEQ ID NO: 1047), CAAguaucuu (SEQ ID NO: 1048), CAAguaugag (SEQ ID NO: 1049), CAAguaugua (SEQ ID NO: 1050), CAAguauguc (SEQ ID NO: 1051), CAAguaugug (SEQ ID NO: 1052), CAAguauguu (SEQ ID NO: 1053), CAAguauuga (SEQ ID NO: 1054), CAAguauuuc (SEQ ID NO: 1055), CAAgucagac (SEQ ID NO: 1056), CAAgucagua (SEQ ID NO: 1057), CAAgucuaua (SEQ ID NO: 1058), CAAgucugau (SEQ ID NO: 1059), CAAgugacuu (SEQ ID NO: 1060), CAAgugagaa (SEQ ID NO: 1061), CAAgugagac (SEQ ID NO: 1062), CAAgugagca (SEQ ID NO: 1063), CAAgugaggc (SEQ ID NO: 1064), CAAgugaggg (SEQ ID NO: 1065), CAAgugagua (SEQ ID NO: 1066), CAAgugaguc (SEQ ID NO: 1067), CAAgugagug (SEQ ID NO: 1068), CAAgugaucc (SEQ ID NO: 1069), CAAgugaucu (SEQ ID NO: 1070), CAAgugauuc (SEQ ID NO: 1071), CAAgugauug (SEQ ID NO: 1072), CAAgugauuu (SEQ ID NO: 1073), CAAgugccuu (SEQ ID NO: 1074), CAAgugggua (SEQ ID NO: 1075), CAAguggguc (SEQ ID NO: 1076), CAAgugggug (SEQ ID NO: 1077), CAAgugugag (SEQ ID NO: 1078), CAAguuaaaa (SEQ ID NO: 1079), CAAguuaagu (SEQ ID NO: 1080), CAAguuaauc (SEQ ID NO: 1081), CAAguuagaa (SEQ ID NO: 1082), CAAguuaguu (SEQ ID NO: 1083), CAAguucaag (SEQ ID NO: 1084), CAAguuccgu (SEQ ID NO: 1085), CAAguuggua (SEQ ID NO: 1086), CAAguuuagu (SEQ ID NO: 1087), CAAguuucca (SEQ ID NO: 1088), CAAguuuguu (SEQ ID NO: 1089), CACguaagag (SEQ ID NO: 1090), CACguaagca (SEQ ID NO: 1091), CACguaauug (SEQ ID NO: 1092), CACguaggac (SEQ ID NO: 1093), CACguaucga (SEQ ID NO: 1094), CACgucaguu (SEQ ID NO: 1095), CACgugagcu (SEQ ID NO: 1096), CACgugaguc (SEQ ID NO: 1097), CACgugagug (SEQ ID NO: 1098), CAGgcaagaa (SEQ ID NO: 1099), CAGgcaagac (SEQ ID NO: 1100), CAGgcaagag (SEQ ID NO: 1101), CAGgcaagga (SEQ ID NO: 1102), CAGgcaagua (SEQ ID NO: 1103), CAGgcaagug (SEQ ID NO: 1104), CAGgcaaguu (SEQ ID NO: 1105), CAGgcacgca (SEQ ID NO: 1106), CAGgcagagg (SEQ ID NO: 1107), CAGgcaggug (SEQ ID NO: 1108), CAGgcaucau (SEQ ID NO: 1109), CAGgcaugaa (SEQ ID NO: 1110), CAGgcaugag (SEQ ID NO: 1111), CAGgcaugca (SEQ ID NO: 1112), CAGgcaugcg (SEQ ID NO: 1113), CAGgcaugug (SEQ ID NO: 1114), CAGgcgagag (SEQ ID NO: 1115), CAGgcgccug (SEQ ID NO: 1116), CAGgcgugug (SEQ ID NO: 1117), CAGguaaaaa (SEQ ID NO: 1118), CAGguaaaag (SEQ ID NO: 1119), CAGguaaaca (SEQ ID NO: 1120), CAGguaaacc (SEQ ID NO: 1121), CAGguaaaga (SEQ ID NO: 1122), CAGguaaagc (SEQ ID NO: 1123), CAGguaaagu (SEQ ID NO: 1124), CAGguaaaua (SEQ ID NO: 1125), CAGguaaauc (SEQ ID NO: 1126), CAGguaaaug (SEQ ID NO: 1127), CAGguaaauu (SEQ ID NO: 1128), CAGguaacag (SEQ ID NO: 1129), CAGguaacau (SEQ ID NO: 1130), CAGguaacca (SEQ ID NO: 1131), CAGguaaccg (SEQ ID NO: 1132), CAGguaacgu (SEQ ID NO: 1133), CAGguaacua (SEQ ID NO: 1134), CAGguaacuc (SEQ ID NO: 1135), CAGguaacug (SEQ ID NO: 1136), CAGguaacuu (SEQ ID NO: 1137), CAGguaagaa (SEQ ID NO: 1138), CAGguaagac (SEQ ID NO: 1139), CAGguaagag (SEQ ID NO: 1140), CAGguaagau (SEQ ID NO: 1141), CAGguaagcc (SEQ ID NO: 1142), CAGguaagga (SEQ ID NO: 1143), CAGguaaggc (SEQ ID NO: 1144), CAGguaaggg (SEQ ID NO: 1145), CAGguaaggu (SEQ ID NO: 1146), CAGguaagua (SEQ ID NO: 1147), CAGguaagug (SEQ ID NO: 1148), CAGguaaguu (SEQ ID NO: 1149), CAGguaauaa (SEQ ID NO: 1150), CAGguaauau (SEQ ID NO: 1151), CAGguaaucc (SEQ ID NO: 1152), CAGguaaugc (SEQ ID NO: 1153), CAGguaaugg (SEQ ID NO: 1154), CAGguaaugu (SEQ ID NO: 1155), CAGguaauua (SEQ ID NO: 1156), CAGguaauuc (SEQ ID NO: 1157), CAGguaauug (SEQ ID NO: 1158), CAGguaauuu (SEQ ID NO: 1159), CAGguacaaa (SEQ ID NO: 1160), CAGguacaag (SEQ ID NO: 1161), CAGguacaau (SEQ ID NO: 1162), CAGguacaca (SEQ ID NO: 1163), CAGguacacg (SEQ ID NO: 1164), CAGguacaga (SEQ ID NO: 1165), CAGguacagg (SEQ ID NO: 1166), CAGguacagu (SEQ ID NO: 1167), CAGguacaua (SEQ ID NO: 1168), CAGguacaug (SEQ ID NO: 1169), CAGguacauu (SEQ ID NO: 1170), CAGguaccac (SEQ ID NO: 1171), CAGguaccca (SEQ ID NO: 1172), CAGguacccg (SEQ ID NO: 1173), CAGguacccu (SEQ ID NO: 1174), CAGguaccgc (SEQ ID NO: 1175), CAGguaccgg (SEQ ID NO: 1176), CAGguaccuc (SEQ ID NO: 1177), CAGguaccug (SEQ ID NO: 1178), CAGguaccuu (SEQ ID NO: 1179), CAGguacgag (SEQ ID NO: 1180), CAGguacgca (SEQ ID NO: 1181), CAGguacgcc (SEQ ID NO: 1182), CAGguacggu (SEQ ID NO: 1183), CAGguacgua (SEQ ID NO: 1184), CAGguacgug (SEQ ID NO: 1185), CAGguacuaa (SEQ ID NO: 1186), CAGguacuag (SEQ ID NO: 1187), CAGguacuau (SEQ ID NO: 1188), CAGguacucc (SEQ ID NO: 1189), CAGguacucu (SEQ ID NO: 1190), CAGguacuga (SEQ ID NO: 1191), CAGguacugc (SEQ ID NO: 1192), CAGguacugu (SEQ ID NO: 1193), CAGguacuua (SEQ ID NO: 1194), CAGguacuuu (SEQ ID NO: 1195), CAGguagaaa (SEQ ID NO: 1196), CAGguagaac (SEQ ID NO: 1197), CAGguagaag (SEQ ID NO: 1198), CAGguagaca (SEQ ID NO: 1199), CAGguagacc (SEQ ID NO: 1200), CAGguagaga (SEQ ID NO: 1201), CAGguagauu (SEQ ID NO: 1202), CAGguagcaa (SEQ ID NO: 1203), CAGguagcac (SEQ ID NO: 1204), CAGguagcag (SEQ ID NO: 1205), CAGguagcca (SEQ ID NO: 1206), CAGguagcgu (SEQ ID NO: 1207), CAGguagcua (SEQ ID NO: 1208), CAGguagcuc (SEQ ID NO: 1209), CAGguagcug (SEQ ID NO: 1210), CAGguagcuu (SEQ ID NO: 1211), CAGguaggaa (SEQ ID NO: 1212), CAGguaggac (SEQ ID NO: 1213), CAGguaggag (SEQ ID NO: 1214), CAGguaggca (SEQ ID NO: 1215), CAGguaggga (SEQ ID NO: 1216), CAGguagggc (SEQ ID NO: 1217), CAGguagggg (SEQ ID NO: 1218), CAGguagggu (SEQ ID NO: 1219), CAGguaggua (SEQ ID NO: 1220), CAGguagguc (SEQ ID NO: 1221), CAGguaggug (SEQ ID NO: 1222), CAGguagguu (SEQ ID NO: 1223), CAGguaguaa (SEQ ID NO: 1224), CAGguaguau (SEQ ID NO: 1225), CAGguaguca (SEQ ID NO: 1226), CAGguagucc (SEQ ID NO: 1227), CAGguaguga (SEQ ID NO: 1228), CAGguagugu (SEQ ID NO: 1229), CAGguaguuc (SEQ ID NO: 1230), CAGguaguug (SEQ ID NO: 1231), CAGguaguuu (SEQ ID NO: 1232), CAGguauaag (SEQ ID NO: 1233), CAGguauaca (SEQ ID NO: 1234), CAGguauaga (SEQ ID NO: 1235), CAGguauauc (SEQ ID NO: 1236), CAGguauaug (SEQ ID NO: 1237), CAGguauauu (SEQ ID NO: 1238), CAGguaucag (SEQ ID NO: 1239), CAGguaucau (SEQ ID NO: 1240), CAGguauccu (SEQ ID NO: 1241), CAGguaucga (SEQ ID NO: 1242), CAGguaucgc (SEQ ID NO: 1243), CAGguaucua (SEQ ID NO: 1244), CAGguaucug (SEQ ID NO: 1245), CAGguaucuu (SEQ ID NO: 1246), CAGguaugaa (SEQ ID NO: 1247), CAGguaugac (SEQ ID NO: 1248), CAGguaugag (SEQ ID NO: 1249), CAGguaugau (SEQ ID NO: 1250), CAGguaugca (SEQ ID NO: 1251), CAGguaugcc (SEQ ID NO: 1252), CAGguaugcg (SEQ ID NO: 1253), CAGguaugcu (SEQ ID NO: 1254), CAGguaugga (SEQ ID NO: 1255), CAGguauggg (SEQ ID NO: 1256), CAGguauggu (SEQ ID NO: 1257), CAGguaugua (SEQ ID NO: 1258), CAGguauguc (SEQ ID NO: 1259), CAGguaugug (SEQ ID NO: 1260), CAGguauguu (SEQ ID NO: 1261), CAGguauuau (SEQ ID NO: 1262), CAGguauuca (SEQ ID NO: 1263), CAGguauucu (SEQ ID NO: 1264), CAGguauuga (SEQ ID NO: 1265), CAGguauugg (SEQ ID NO: 1266), CAGguauugu (SEQ ID NO: 1267), CAGguauuua (SEQ ID NO: 1268), CAGguauuuc (SEQ ID NO: 1269), CAGguauuug (SEQ ID NO: 1270), CAGguauuuu (SEQ ID NO: 1271), CAGgucaaca (SEQ ID NO: 1272), CAGgucaaug (SEQ ID NO: 1273), CAGgucacgu (SEQ ID NO: 1274), CAGgucagaa (SEQ ID NO: 1275), CAGgucagac (SEQ ID NO: 1276), CAGgucagca (SEQ ID NO: 1277), CAGgucagcc (SEQ ID NO: 1278), CAGgucagcg (SEQ ID NO: 1279), CAGgucagga (SEQ ID NO: 1280), CAGgucagua (SEQ ID NO: 1281), CAGgucaguc (SEQ ID NO: 1282), CAGgucagug (SEQ ID NO: 1283), CAGgucaguu (SEQ ID NO: 1284), CAGgucaucc (SEQ ID NO: 1285), CAGgucaugc (SEQ ID NO: 1286), CAGgucauua (SEQ ID NO: 1287), CAGgucauuu (SEQ ID NO: 1288), CAGguccacc (SEQ ID NO: 1289), CAGguccacu (SEQ ID NO: 1290), CAGguccagu (SEQ ID NO: 1291), CAGguccauc (SEQ ID NO: 1292), CAGguccauu (SEQ ID NO: 1293), CAGgucccag (SEQ ID NO: 1294), CAGgucccug (SEQ ID NO: 1295), CAGguccuga (SEQ ID NO: 1296), CAGguccugc (SEQ ID NO: 1297), CAGguccugg (SEQ ID NO: 1298), CAGgucggcc (SEQ ID NO: 1299), CAGgucggug (SEQ ID NO: 1300), CAGgucguug (SEQ ID NO: 1301), CAGgucucuc (SEQ ID NO: 1302), CAGgucucuu (SEQ ID NO: 1303), CAGgucugag (SEQ ID NO: 1304), CAGgucugcc (SEQ ID NO: 1305), CAGgucugcg (SEQ ID NO: 1306), CAGgucugga (SEQ ID NO: 1307), CAGgucuggu (SEQ ID NO: 1308), CAGgucugua (SEQ ID NO: 1309), CAGgucuguc (SEQ ID NO: 1310), CAGgucugug (SEQ ID NO: 1311), CAGgucuguu (SEQ ID NO: 1312), CAGgucuucc (SEQ ID NO: 1313), CAGgucuuuc (SEQ ID NO: 1314), CAGgugaaag (SEQ ID NO: 1315), CAGgugaaau (SEQ ID NO: 1316), CAGgugaaca (SEQ ID NO: 1317), CAGgugaaga (SEQ ID NO: 1318), CAGgugaagg (SEQ ID NO: 1319), CAGgugaaua (SEQ ID NO: 1320), CAGgugaauc (SEQ ID NO: 1321), CAGgugaauu (SEQ ID NO: 1322), CAGgugacaa (SEQ ID NO: 1323), CAGgugacau (SEQ ID NO: 1324), CAGgugacca (SEQ ID NO: 1325), CAGgugaccc (SEQ ID NO: 1326), CAGgugaccg (SEQ ID NO: 1327), CAGgugaccu (SEQ ID NO: 1328), CAGgugacgg (SEQ ID NO: 1329), CAGgugacua (SEQ ID NO: 1330), CAGgugacuc (SEQ ID NO: 1331), CAGgugacug (SEQ ID NO: 1332), CAGgugagaa (SEQ ID NO: 1333), CAGgugagac (SEQ ID NO: 1334), CAGgugagag (SEQ ID NO: 1335), CAGgugagau (SEQ ID NO: 1336), CAGgugagca (SEQ ID NO: 1337), CAGgugagcc (SEQ ID NO: 1338), CAGgugagcg (SEQ ID NO: 1339), CAGgugagcu (SEQ ID NO: 1340), CAGgugagga (SEQ ID NO: 1341), CAGgugaggc (SEQ ID NO: 1342), CAGgugaggg (SEQ ID NO: 1343), CAGgugaggu (SEQ ID NO: 1344), CAGgugagua (SEQ ID NO: 1345), CAGgugaguc (SEQ ID NO: 1346), CAGgugagug (SEQ ID NO: 1347), CAGgugaguu (SEQ ID NO: 1348), CAGgugauaa (SEQ ID NO: 1349), CAGgugaucc (SEQ ID NO: 1350), CAGgugaucu (SEQ ID NO: 1351), CAGgugaugc (SEQ ID NO: 1352), CAGgugaugg (SEQ ID NO: 1353), CAGgugaugu (SEQ ID NO: 1354), CAGgugauua (SEQ ID NO: 1355), CAGgugauuc (SEQ ID NO: 1356), CAGgugauug (SEQ ID NO: 1357), CAGgugauuu (SEQ ID NO: 1358), CAGgugcaaa (SEQ ID NO: 1359), CAGgugcaag (SEQ ID NO: 1360), CAGgugcaca (SEQ ID NO: 1361), CAGgugcacg (SEQ ID NO: 1362), CAGgugcaga (SEQ ID NO: 1363), CAGgugcagg (SEQ ID NO: 1364), CAGgugcaua (SEQ ID NO: 1365), CAGgugcauc (SEQ ID NO: 1366), CAGgugcaug (SEQ ID NO: 1367), CAGgugccaa (SEQ ID NO: 1368), CAGgugccca (SEQ ID NO: 1369), CAGgugcccc (SEQ ID NO: 1370), CAGgugcccg (SEQ ID NO: 1371), CAGgugccua (SEQ ID NO: 1372), CAGgugccug (SEQ ID NO: 1373), CAGgugcgaa (SEQ ID NO: 1374), CAGgugcgca (SEQ ID NO: 1375), CAGgugcgcc (SEQ ID NO: 1376), CAGgugcgcg (SEQ ID NO: 1377), CAGgugcgga (SEQ ID NO: 1378), CAGgugcggu (SEQ ID NO: 1379), CAGgugcgua (SEQ ID NO: 1380), CAGgugcguc (SEQ ID NO: 1381), CAGgugcgug (SEQ ID NO: 1382), CAGgugcuag (SEQ ID NO: 1383), CAGgugcuau (SEQ ID NO: 1384), CAGgugcuca (SEQ ID NO: 1385), CAGgugcucc (SEQ ID NO: 1386), CAGgugcucg (SEQ ID NO: 1387), CAGgugcugc (SEQ ID NO: 1388), CAGgugcugg (SEQ ID NO: 1389), CAGgugcuua (SEQ ID NO: 1390), CAGgugcuuc (SEQ ID NO: 1391), CAGgugcuug (SEQ ID NO: 1392), CAGguggaac (SEQ ID NO: 1393), CAGguggaag (SEQ ID NO: 1394), CAGguggaau (SEQ ID NO: 1395), CAGguggaga (SEQ ID NO: 1396), CAGguggagu (SEQ ID NO: 1397), CAGguggauu (SEQ ID NO: 1398), CAGguggcca (SEQ ID NO: 1399), CAGguggcuc (SEQ ID NO: 1400), CAGguggcug (SEQ ID NO: 1401), CAGgugggaa (SEQ ID NO: 1402), CAGgugggac (SEQ ID NO: 1403), CAGgugggag (SEQ ID NO: 1404), CAGgugggau (SEQ ID NO: 1405), CAGgugggca (SEQ ID NO: 1406), CAGgugggcc (SEQ ID NO: 1407), CAGgugggcu (SEQ ID NO: 1408), CAGgugggga (SEQ ID NO: 1409), CAGguggggc (SEQ ID NO: 1410), CAGguggggg (SEQ ID NO: 1411), CAGguggggu (SEQ ID NO: 1412), CAGgugggua (SEQ ID NO: 1413), CAGguggguc (SEQ ID NO: 1414), CAGgugggug (SEQ ID NO: 1415), CAGguggguu (SEQ ID NO: 1416), CAGguggucu (SEQ ID NO: 1417), CAGguggugg (SEQ ID NO: 1418), CAGgugguug (SEQ ID NO: 1419), CAGguguaca (SEQ ID NO: 1420), CAGguguagg (SEQ ID NO: 1421), CAGguguauc (SEQ ID NO: 1422), CAGgugucac (SEQ ID NO: 1423), CAGgugucag (SEQ ID NO: 1424), CAGgugucca (SEQ ID NO: 1425), CAGguguccu (SEQ ID NO: 1426), CAGgugucua (SEQ ID NO: 1427), CAGgugucuc (SEQ ID NO: 1428), CAGgugucug (SEQ ID NO: 1429), CAGgugugaa (SEQ ID NO: 1430), CAGgugugac (SEQ ID NO: 1431), CAGgugugag (SEQ ID NO: 1432), CAGgugugau (SEQ ID NO: 1433), CAGgugugca (SEQ ID NO: 1434), CAGgugugcc (SEQ ID NO: 1435), CAGgugugcg (SEQ ID NO: 1436), CAGgugugcu (SEQ ID NO: 1437), CAGgugugga (SEQ ID NO: 1438), CAGguguggc (SEQ ID NO: 1439), CAGgugugua (SEQ ID NO: 1440), CAGguguguc (SEQ ID NO: 1441), CAGgugugug (SEQ ID NO: 1442), CAGguguguu (SEQ ID NO: 1443), CAGguguuua (SEQ ID NO: 1444), CAGguuaaaa (SEQ ID NO: 1445), CAGguuaaua (SEQ ID NO: 1446), CAGguuaauc (SEQ ID NO: 1447), CAGguuaccu (SEQ ID NO: 1448), CAGguuagaa (SEQ ID NO: 1449), CAGguuagag (SEQ ID NO: 1450), CAGguuagau (SEQ ID NO: 1451), CAGguuagcc (SEQ ID NO: 1452), CAGguuaggg (SEQ ID NO: 1453), CAGguuaggu (SEQ ID NO: 1454), CAGguuagua (SEQ ID NO: 1455), CAGguuaguc (SEQ ID NO: 1456), CAGguuagug (SEQ ID NO: 1457), CAGguuaguu (SEQ ID NO: 1458), CAGguuauca (SEQ ID NO: 1459), CAGguuaugu (SEQ ID NO: 1460), CAGguuauua (SEQ ID NO: 1461), CAGguuauug (SEQ ID NO: 1462), CAGguucaaa (SEQ ID NO: 1463), CAGguucaac (SEQ ID NO: 1464), CAGguucaag (SEQ ID NO: 1465), CAGguucaca (SEQ ID NO: 1466), CAGguucacg (SEQ ID NO: 1467), CAGguucagg (SEQ ID NO: 1468), CAGguucaug (SEQ ID NO: 1469), CAGguuccag (SEQ ID NO: 1470), CAGguuccca (SEQ ID NO: 1471), CAGguucccg (SEQ ID NO: 1472), CAGguucgaa (SEQ ID NO: 1473), CAGguucgag (SEQ ID NO: 1474), CAGguucuau (SEQ ID NO: 1475), CAGguucugc (SEQ ID NO: 1476), CAGguucuua (SEQ ID NO: 1477), CAGguucuuc (SEQ ID NO: 1478), CAGguucuuu (SEQ ID NO: 1479), CAGguugaac (SEQ ID NO: 1480), CAGguugaag (SEQ ID NO: 1481), CAGguugagu (SEQ ID NO: 1482), CAGguugaua (SEQ ID NO: 1483), CAGguuggag (SEQ ID NO: 1484), CAGguuggca (SEQ ID NO: 1485), CAGguuggcc (SEQ ID NO: 1486), CAGguugguc (SEQ ID NO: 1487), CAGguuggug (SEQ ID NO: 1488), CAGguugguu (SEQ ID NO: 1489), CAGguuguaa (SEQ ID NO: 1490), CAGguuguac (SEQ ID NO: 1491), CAGguuguau (SEQ ID NO: 1492), CAGguuguca (SEQ ID NO: 1493), CAGguuguga (SEQ ID NO: 1494), CAGguuguug (SEQ ID NO: 1495), CAGguuuaag (SEQ ID NO: 1496), CAGguuuacc (SEQ ID NO: 1497), CAGguuuagc (SEQ ID NO: 1498), CAGguuuagu (SEQ ID NO: 1499), CAGguuucuu (SEQ ID NO: 1500), CAGguuugaa (SEQ ID NO: 1501), CAGguuugag (SEQ ID NO: 1502), CAGguuugau (SEQ ID NO: 1503), CAGguuugcc (SEQ ID NO: 1504), CAGguuugcu (SEQ ID NO: 1505), CAGguuuggg (SEQ ID NO: 1506), CAGguuuggu (SEQ ID NO: 1507), CAGguuugua (SEQ ID NO: 1508), CAGguuugug (SEQ ID NO: 1509), CAGguuuguu (SEQ ID NO: 1510), CAGguuuucu (SEQ ID NO: 1511), CAGguuuugg (SEQ ID NO: 1512), CAGguuuuuc (SEQ ID NO: 1513), CAGguuuuuu (SEQ ID NO: 1514), CAUgcagguu (SEQ ID NO: 1515), CAUguaaaac (SEQ ID NO: 1516), CAUguaacua (SEQ ID NO: 1517), CAUguaagaa (SEQ ID NO: 1518), CAUguaagag (SEQ ID NO: 1519), CAUguaagau (SEQ ID NO: 1520), CAUguaagcc (SEQ ID NO: 1521), CAUguaagua (SEQ ID NO: 1522), CAUguaagug (SEQ ID NO: 1523), CAUguaaguu (SEQ ID NO: 1524), CAUguaauua (SEQ ID NO: 1525), CAUguacaua (SEQ ID NO: 1526), CAUguaccac (SEQ ID NO: 1527), CAUguacguu (SEQ ID NO: 1528), CAUguaggua (SEQ ID NO: 1529), CAUguaggug (SEQ ID NO: 1530), CAUguagguu (SEQ ID NO: 1531), CAUguaugaa (SEQ ID NO: 1532), CAUguaugua (SEQ ID NO: 1533), CAUguaugug (SEQ ID NO: 1534), CAUguauguu (SEQ ID NO: 1535), CAUgugagaa (SEQ ID NO: 1536), CAUgugagca (SEQ ID NO: 1537), CAUgugagcu (SEQ ID NO: 1538), CAUgugagua (SEQ ID NO: 1539), CAUgugaguc (SEQ ID NO: 1540), CAUgugagug (SEQ ID NO: 1541), CAUgugaguu (SEQ ID NO: 1542), CAUgugcgua (SEQ ID NO: 1543), CAUgugggaa (SEQ ID NO: 1544), CAUguggguu (SEQ ID NO: 1545), CAUgugugug (SEQ ID NO: 1546), CAUguguguu (SEQ ID NO: 1547), CAUguuaaua (SEQ ID NO: 1548), CAUguuagcc (SEQ ID NO: 1549), CCAguaagau (SEQ ID NO: 1550), CCAguaagca (SEQ ID NO: 1551), CCAguaagcc (SEQ ID NO: 1552), CCAguaagcu (SEQ ID NO: 1553), CCAguaagga (SEQ ID NO: 1554), CCAguaagua (SEQ ID NO: 1555), CCAguaaguc (SEQ ID NO: 1556), CCAguaagug (SEQ ID NO: 1557), CCAguaaguu (SEQ ID NO: 1558), CCAguaauug (SEQ ID NO: 1559), CCAguacggg (SEQ ID NO: 1560), CCAguagguc (SEQ ID NO: 1561), CCAguauugu (SEQ ID NO: 1562), CCAgugaggc (SEQ ID NO: 1563), CCAgugagua (SEQ ID NO: 1564), CCAgugagug (SEQ ID NO: 1565), CCAguggguc (SEQ ID NO: 1566), CCAguuaguu (SEQ ID NO: 1567), CCAguugagu (SEQ ID NO: 1568), CCCguaagau (SEQ ID NO: 1569), CCCguauguc (SEQ ID NO: 1570), CCCguauguu (SEQ ID NO: 1571), CCCguccugc (SEQ ID NO: 1572), CCCgugagug (SEQ ID NO: 1573), CCGguaaaga (SEQ ID NO: 1574), CCGguaagau (SEQ ID NO: 1575), CCGguaagcc (SEQ ID NO: 1576), CCGguaagga (SEQ ID NO: 1577), CCGguaaggc (SEQ ID NO: 1578), CCGguaaugg (SEQ ID NO: 1579), CCGguacagu (SEQ ID NO: 1580), CCGguacuga (SEQ ID NO: 1581), CCGguauucc (SEQ ID NO: 1582), CCGgucagug (SEQ ID NO: 1583), CCGgugaaaa (SEQ ID NO: 1584), CCGgugagaa (SEQ ID NO: 1585), CCGgugaggg (SEQ ID NO: 1586), CCGgugagug (SEQ ID NO: 1587), CCGgugaguu (SEQ ID NO: 1588), CCGgugcgcg (SEQ ID NO: 1589), CCGgugggcg (SEQ ID NO: 1590), CCGguugguc (SEQ ID NO: 1591), CCUguaaaug (SEQ ID NO: 1592), CCUguaaauu (SEQ ID NO: 1593), CCUguaagaa (SEQ ID NO: 1594), CCUguaagac (SEQ ID NO: 1595), CCUguaagag (SEQ ID NO: 1596), CCUguaagca (SEQ ID NO: 1597), CCUguaagcg (SEQ ID NO: 1598), CCUguaagga (SEQ ID NO: 1599), CCUguaaguu (SEQ ID NO: 1600), CCUguaggua (SEQ ID NO: 1601), CCUguaggug (SEQ ID NO: 1602), CCUguaucuu (SEQ ID NO: 1603), CCUguauggu (SEQ ID NO: 1604), CCUguaugug (SEQ ID NO: 1605), CCUgugagaa (SEQ ID NO: 1606), CCUgugagca (SEQ ID NO: 1607), CCUgugaggg (SEQ ID NO: 1608), CCUgugaguc (SEQ ID NO: 1609), CCUgugagug (SEQ ID NO: 1610), CCUgugaguu (SEQ ID NO: 1611), CCUguggcuc (SEQ ID NO: 1612), CCUgugggua (SEQ ID NO: 1613), CCUgugugua (SEQ ID NO: 1614), CCUguuagaa (SEQ ID NO: 1615), CGAguaaggg (SEQ ID NO: 1616), CGAguaaggu (SEQ ID NO: 1617), CGAguagcug (SEQ ID NO: 1618), CGAguaggug (SEQ ID NO: 1619), CGAguagguu (SEQ ID NO: 1620), CGAgugagca (SEQ ID NO: 1621), CGCguaagag (SEQ ID NO: 1622), CGGgcaggca (SEQ ID NO: 1623), CGGguaagcc (SEQ ID NO: 1624), CGGguaagcu (SEQ ID NO: 1625), CGGguaaguu (SEQ ID NO: 1626), CGGguaauuc (SEQ ID NO: 1627), CGGguaauuu (SEQ ID NO: 1628), CGGguacagu (SEQ ID NO: 1629), CGGguacggg (SEQ ID NO: 1630), CGGguaggag (SEQ ID NO: 1631), CGGguaggcc (SEQ ID NO: 1632), CGGguaggug (SEQ ID NO: 1633), CGGguauuua (SEQ ID NO: 1634), CGGgucugag (SEQ ID NO: 1635), CGGgugaccg (SEQ ID NO: 1636), CGGgugacuc (SEQ ID NO: 1637), CGGgugagaa (SEQ ID NO: 1638), CGGgugaggg (SEQ ID NO: 1639), CGGgugaggu (SEQ ID NO: 1640), CGGgugagua (SEQ ID NO: 1641), CGGgugagug (SEQ ID NO: 1642), CGGgugaguu (SEQ ID NO: 1643), CGGgugauuu (SEQ ID NO: 1644), CGGgugccuu (SEQ ID NO: 1645), CGGgugggag (SEQ ID NO: 1646), CGGgugggug (SEQ ID NO: 1647), CGGguggguu (SEQ ID NO: 1648), CGGguguguc (SEQ ID NO: 1649), CGGgugugug (SEQ ID NO: 1650), CGGguguguu (SEQ ID NO: 1651), CGGguucaag (SEQ ID NO: 1652), CGGguucaug (SEQ ID NO: 1653), CGGguuugcu (SEQ ID NO: 1654), CGUguagggu (SEQ ID NO: 1655), CGUguaugca (SEQ ID NO: 1656), CGUguaugua (SEQ ID NO: 1657), CGUgucugua (SEQ ID NO: 1658), CGUgugagug (SEQ ID NO: 1659), CGUguuuucu (SEQ ID NO: 1660), CUAguaaaug (SEQ ID NO: 1661), CUAguaagcg (SEQ ID NO: 1662), CUAguaagcu (SEQ ID NO: 1663), CUAguaagua (SEQ ID NO: 1664), CUAguaaguc (SEQ ID NO: 1665), CUAguaagug (SEQ ID NO: 1666), CUAguaaguu (SEQ ID NO: 1667), CUAguaauuu (SEQ ID NO: 1668), CUAguaggua (SEQ ID NO: 1669), CUAguagguu (SEQ ID NO: 1670), CUAguaugua (SEQ ID NO: 1671), CUAguauguu (SEQ ID NO: 1672), CUAgugagua (SEQ ID NO: 1673), CUCguaagca (SEQ ID NO: 1674), CUCguaagug (SEQ ID NO: 1675), CUCguaaguu (SEQ ID NO: 1676), CUCguaucug (SEQ ID NO: 1677), CUCgucugug (SEQ ID NO: 1678), CUCgugaaua (SEQ ID NO: 1679), CUCgugagua (SEQ ID NO: 1680), CUCgugauua (SEQ ID NO: 1681), CUGguaaaaa (SEQ ID NO: 1682), CUGguaaaau (SEQ ID NO: 1683), CUGguaaacc (SEQ ID NO: 1684), CUGguaaacg (SEQ ID NO: 1685), CUGguaaagc (SEQ ID NO: 1686), CUGguaaaua (SEQ ID NO: 1687), CUGguaaauc (SEQ ID NO: 1688), CUGguaaaug (SEQ ID NO: 1689), CUGguaaauu (SEQ ID NO: 1690), CUGguaacac (SEQ ID NO: 1691), CUGguaacag (SEQ ID NO: 1692), CUGguaaccc (SEQ ID NO: 1693), CUGguaaccg (SEQ ID NO: 1694), CUGguaacug (SEQ ID NO: 1695), CUGguaacuu (SEQ ID NO: 1696), CUGguaagaa (SEQ ID NO: 1697), CUGguaagag (SEQ ID NO: 1698), CUGguaagau (SEQ ID NO: 1699), CUGguaagca (SEQ ID NO: 1700), CUGguaagcc (SEQ ID NO: 1701), CUGguaagcu (SEQ ID NO: 1702), CUGguaagga (SEQ ID NO: 1703), CUGguaaggc (SEQ ID NO: 1704), CUGguaaggg (SEQ ID NO: 1705), CUGguaaggu (SEQ ID NO: 1706), CUGguaagua (SEQ ID NO: 1707), CUGguaagug (SEQ ID NO: 1708), CUGguaaguu (SEQ ID NO: 1709), CUGguaauga (SEQ ID NO: 1710), CUGguaaugc (SEQ ID NO: 1711), CUGguaauuc (SEQ ID NO: 1712), CUGguaauuu (SEQ ID NO: 1713), CUGguacaac (SEQ ID NO: 1714), CUGguacaau (SEQ ID NO: 1715), CUGguacaga (SEQ ID NO: 1716), CUGguacaua (SEQ ID NO: 1717), CUGguacauu (SEQ ID NO: 1718), CUGguaccau (SEQ ID NO: 1719), CUGguacguu (SEQ ID NO: 1720), CUGguacuaa (SEQ ID NO: 1721), CUGguacuug (SEQ ID NO: 1722), CUGguacuuu (SEQ ID NO: 1723), CUGguagaga (SEQ ID NO: 1724), CUGguagaua (SEQ ID NO: 1725), CUGguagcgu (SEQ ID NO: 1726), CUGguaggau (SEQ ID NO: 1727), CUGguaggca (SEQ ID NO: 1728), CUGguaggua (SEQ ID NO: 1729), CUGguagguc (SEQ ID NO: 1730), CUGguaggug (SEQ ID NO: 1731), CUGguaucaa (SEQ ID NO: 1732), CUGguaugau (SEQ ID NO: 1733), CUGguauggc (SEQ ID NO: 1734), CUGguauggu (SEQ ID NO: 1735), CUGguaugua (SEQ ID NO: 1736), CUGguaugug (SEQ ID NO: 1737), CUGguauguu (SEQ ID NO: 1738), CUGguauuga (SEQ ID NO: 1739), CUGguauuuc (SEQ ID NO: 1740), CUGguauuuu (SEQ ID NO: 1741), CUGgucaaca (SEQ ID NO: 1742), CUGgucagag (SEQ ID NO: 1743), CUGgucccgc (SEQ ID NO: 1744), CUGgucggua (SEQ ID NO: 1745), CUGgucuggg (SEQ ID NO: 1746), CUGgugaagu (SEQ ID NO: 1747), CUGgugaaua (SEQ ID NO: 1748), CUGgugaauu (SEQ ID NO: 1749), CUGgugacua (SEQ ID NO: 1750), CUGgugagaa (SEQ ID NO: 1751), CUGgugagac (SEQ ID NO: 1752), CUGgugagca (SEQ ID NO: 1753), CUGgugagcu (SEQ ID NO: 1754), CUGgugagga (SEQ ID NO: 1755), CUGgugaggc (SEQ ID NO: 1756), CUGgugaggg (SEQ ID NO: 1757), CUGgugaggu (SEQ ID NO: 1758), CUGgugagua (SEQ ID NO: 1759), CUGgugaguc (SEQ ID NO: 1760), CUGgugagug (SEQ ID NO: 1761), CUGgugaguu (SEQ ID NO: 1762), CUGgugauua (SEQ ID NO: 1763), CUGgugauuu (SEQ ID NO: 1764), CUGgugcaga (SEQ ID NO: 1765), CUGgugcgcu (SEQ ID NO: 1766), CUGgugcgug (SEQ ID NO: 1767), CUGgugcuga (SEQ ID NO: 1768), CUGgugggag (SEQ ID NO: 1769), CUGgugggga (SEQ ID NO: 1770), CUGgugggua (SEQ ID NO: 1771), CUGguggguc (SEQ ID NO: 1772), CUGgugggug (SEQ ID NO: 1773), CUGguggguu (SEQ ID NO: 1774), CUGgugugaa (SEQ ID NO: 1775), CUGgugugca (SEQ ID NO: 1776), CUGgugugcu (SEQ ID NO: 1777), CUGguguggu (SEQ ID NO: 1778), CUGgugugug (SEQ ID NO: 1779), CUGguguguu (SEQ ID NO: 1780), CUGguuagcu (SEQ ID NO: 1781), CUGguuagug (SEQ ID NO: 1782), CUGguucgug (SEQ ID NO: 1783), CUGguuggcu (SEQ ID NO: 1784), CUGguuguuu (SEQ ID NO: 1785), CUGguuugua (SEQ ID NO: 1786), CUGguuuguc (SEQ ID NO: 1787), CUGguuugug (SEQ ID NO: 1788), CUUguaaaug (SEQ ID NO: 1789), CUUguaagcu (SEQ ID NO: 1790), CUUguaagga (SEQ ID NO: 1791), CUUguaaggc (SEQ ID NO: 1792), CUUguaagua (SEQ ID NO: 1793), CUUguaagug (SEQ ID NO: 1794), CUUguaaguu (SEQ ID NO: 1795), CUUguacguc (SEQ ID NO: 1796), CUUguacgug (SEQ ID NO: 1797), CUUguaggua (SEQ ID NO: 1798), CUUguagugc (SEQ ID NO: 1799), CUUguauagg (SEQ ID NO: 1800), CUUgucagua (SEQ ID NO: 1801), CUUgugagua (SEQ ID NO: 1802), CUUgugaguc (SEQ ID NO: 1803), CUUgugaguu (SEQ ID NO: 1804), CUUguggguu (SEQ ID NO: 1805), CUUgugugua (SEQ ID NO: 1806), CUUguuagug (SEQ ID NO: 1807), CUUguuugag (SEQ ID NO: 1808), GAAguaaaac (SEQ ID NO: 1809), GAAguaaagc (SEQ ID NO: 1810), GAAguaaagu (SEQ ID NO: 1811), GAAguaaaua (SEQ ID NO: 1812), GAAguaaauu (SEQ ID NO: 1813), GAAguaagaa (SEQ ID NO: 1814), GAAguaagcc (SEQ ID NO: 1815), GAAguaagcu (SEQ ID NO: 1816), GAAguaagga (SEQ ID NO: 1817), GAAguaagua (SEQ ID NO: 1818), GAAguaagug (SEQ ID NO: 1819), GAAguaaguu (SEQ ID NO: 1820), GAAguaauau (SEQ ID NO: 1821), GAAguaaugc (SEQ ID NO: 1822), GAAguaauua (SEQ ID NO: 1823), GAAguaauuu (SEQ ID NO: 1824), GAAguaccau (SEQ ID NO: 1825), GAAguacgua (SEQ ID NO: 1826), GAAguacguc (SEQ ID NO: 1827), GAAguaggca (SEQ ID NO: 1828), GAAguagguc (SEQ ID NO: 1829), GAAguauaaa (SEQ ID NO: 1830), GAAguaugcu (SEQ ID NO: 1831), GAAguaugug (SEQ ID NO: 1832), GAAguauguu (SEQ ID NO: 1833), GAAguauuaa (SEQ ID NO: 1834), GAAgucagug (SEQ ID NO: 1835), GAAgugagag (SEQ ID NO: 1836), GAAgugagcg (SEQ ID NO: 1837), GAAgugaggu (SEQ ID NO: 1838), GAAgugaguc (SEQ ID NO: 1839), GAAgugagug (SEQ ID NO: 1840), GAAgugaguu (SEQ ID NO: 1841), GAAgugauaa (SEQ ID NO: 1842), GAAgugauuc (SEQ ID NO: 1843), GAAgugcgug (SEQ ID NO: 1844), GAAguguggg (SEQ ID NO: 1845), GAAguguguc (SEQ ID NO: 1846), GAAguuggug (SEQ ID NO: 1847), GACguaaagu (SEQ ID NO: 1848), GACguaagcu (SEQ ID NO: 1849), GACguaagua (SEQ ID NO: 1850), GACguaaugg (SEQ ID NO: 1851), GACguaugcc (SEQ ID NO: 1852), GACguauguu (SEQ ID NO: 1853), GACgugagcc (SEQ ID NO: 1854), GACgugagug (SEQ ID NO: 1855), GAGgcaaaug (SEQ ID NO: 1856), GAGgcaagag (SEQ ID NO: 1857), GAGgcaagua (SEQ ID NO: 1858), GAGgcaagug (SEQ ID NO: 1859), GAGgcaaguu (SEQ ID NO: 1860), GAGgcacgag (SEQ ID NO: 1861), GAGgcaggga (SEQ ID NO: 1862), GAGgcaugug (SEQ ID NO: 1863), GAGgcgaagg (SEQ ID NO: 1864), GAGguaaaaa (SEQ ID NO: 1865), GAGguaaaac (SEQ ID NO: 1866), GAGguaaaag (SEQ ID NO: 1867), GAGguaaaau (SEQ ID NO: 1868), GAGguaaacc (SEQ ID NO: 1869), GAGguaaaga (SEQ ID NO: 1870), GAGguaaagc (SEQ ID NO: 1871), GAGguaaagu (SEQ ID NO: 1872), GAGguaaaua (SEQ ID NO: 1873), GAGguaaauc (SEQ ID NO: 1874), GAGguaaaug (SEQ ID NO: 1875), GAGguaaauu (SEQ ID NO: 1876), GAGguaacaa (SEQ ID NO: 1877), GAGguaacag (SEQ ID NO: 1878), GAGguaacca (SEQ ID NO: 1879), GAGguaaccu (SEQ ID NO: 1880), GAGguaacuu (SEQ ID NO: 1881), GAGguaagaa (SEQ ID NO: 1882), GAGguaagag (SEQ ID NO: 1883), GAGguaagau (SEQ ID NO: 1884), GAGguaagca (SEQ ID NO: 1885), GAGguaagcc (SEQ ID NO: 1886), GAGguaagcg (SEQ ID NO: 1887), GAGguaagcu (SEQ ID NO: 1888), GAGguaagga (SEQ ID NO: 1889), GAGguaaggc (SEQ ID NO: 1890), GAGguaaggg (SEQ ID NO: 1891), GAGguaaggu (SEQ ID NO: 1892), GAGguaagua (SEQ ID NO: 1893), GAGguaaguc (SEQ ID NO: 1894), GAGguaauaa (SEQ ID NO: 1895), GAGguaauac (SEQ ID NO: 1896), GAGguaauau (SEQ ID NO: 1897), GAGguaauca (SEQ ID NO: 1898), GAGguaaucu (SEQ ID NO: 1899), GAGguaaugg (SEQ ID NO: 1900), GAGguaaugu (SEQ ID NO: 1901), GAGguaauug (SEQ ID NO: 1902), GAGguaauuu (SEQ ID NO: 1903), GAGguacaaa (SEQ ID NO: 1904), GAGguacaac (SEQ ID NO: 1905), GAGguacaga (SEQ ID NO: 1906), GAGguacagc (SEQ ID NO: 1907), GAGguacagu (SEQ ID NO: 1908), GAGguacaua (SEQ ID NO: 1909), GAGguacauu (SEQ ID NO: 1910), GAGguaccag (SEQ ID NO: 1911), GAGguaccga (SEQ ID NO: 1912), GAGguaccug (SEQ ID NO: 1913), GAGguaccuu (SEQ ID NO: 1914), GAGguacuag (SEQ ID NO: 1915), GAGguacuau (SEQ ID NO: 1916), GAGguacucc (SEQ ID NO: 1917), GAGguacugc (SEQ ID NO: 1918), GAGguacugg (SEQ ID NO: 1919), GAGguacugu (SEQ ID NO: 1920), GAGguacuug (SEQ ID NO: 1921), GAGguacuuu (SEQ ID NO: 1922), GAGguagaag (SEQ ID NO: 1923), GAGguagaga (SEQ ID NO: 1924), GAGguagagg (SEQ ID NO: 1925), GAGguagagu (SEQ ID NO: 1926), GAGguagauc (SEQ ID NO: 1927), GAGguagcua (SEQ ID NO: 1928), GAGguagcug (SEQ ID NO: 1929), GAGguaggaa (SEQ ID NO: 1930), GAGguaggag (SEQ ID NO: 1931), GAGguaggca (SEQ ID NO: 1932), GAGguaggcu (SEQ ID NO: 1933), GAGguaggga (SEQ ID NO: 1934), GAGguagggc (SEQ ID NO: 1935), GAGguagggg (SEQ ID NO: 1936), GAGguaggua (SEQ ID NO: 1937), GAGguaggug (SEQ ID NO: 1938), GAGguagguu (SEQ ID NO: 1939), GAGguaguaa (SEQ ID NO: 1940), GAGguaguag (SEQ ID NO: 1941), GAGguaguau (SEQ ID NO: 1942), GAGguagucu (SEQ ID NO: 1943), GAGguagugc (SEQ ID NO: 1944), GAGguagugg (SEQ ID NO: 1945), GAGguaguua (SEQ ID NO: 1946), GAGguaguug (SEQ ID NO: 1947), GAGguauaag (SEQ ID NO: 1948), GAGguauacu (SEQ ID NO: 1949), GAGguauagc (SEQ ID NO: 1950), GAGguauaug (SEQ ID NO: 1951), GAGguauauu (SEQ ID NO: 1952), GAGguaucau (SEQ ID NO: 1953), GAGguaucug (SEQ ID NO: 1954), GAGguaucuu (SEQ ID NO: 1955), GAGguaugaa (SEQ ID NO: 1956), GAGguaugac (SEQ ID NO: 1957), GAGguaugag (SEQ ID NO: 1958), GAGguaugcc (SEQ ID NO: 1959), GAGguaugcg (SEQ ID NO: 1960), GAGguaugcu (SEQ ID NO: 1961), GAGguaugga (SEQ ID NO: 1962), GAGguauggg (SEQ ID NO: 1963), GAGguauggu (SEQ ID NO: 1964), GAGguaugua (SEQ ID NO: 1965), GAGguauguc (SEQ ID NO: 1966), GAGguaugug (SEQ ID NO: 1967), GAGguauguu (SEQ ID NO: 1968), GAGguauucc (SEQ ID NO: 1969), GAGguauuga (SEQ ID NO: 1970), GAGguauugu (SEQ ID NO: 1971), GAGguauuua (SEQ ID NO: 1972), GAGguauuuc (SEQ ID NO: 1973), GAGguauuug (SEQ ID NO: 1974), GAGguauuuu (SEQ ID NO: 1975), GAGgucaaca (SEQ ID NO: 1976), GAGgucaagg (SEQ ID NO: 1977), GAGgucaaug (SEQ ID NO: 1978), GAGgucacug (SEQ ID NO: 1979), GAGgucagaa (SEQ ID NO: 1980), GAGgucagag (SEQ ID NO: 1981), GAGgucagcu (SEQ ID NO: 1982), GAGgucagga (SEQ ID NO: 1983), GAGgucaggc (SEQ ID NO: 1984), GAGgucaggg (SEQ ID NO: 1985), GAGgucaggu (SEQ ID NO: 1986), GAGgucagua (SEQ ID NO: 1987), GAGgucauau (SEQ ID NO: 1988), GAGgucaugu (SEQ ID NO: 1989), GAGgucauuu (SEQ ID NO: 1990), GAGguccaua (SEQ ID NO: 1991), GAGguccauc (SEQ ID NO: 1992), GAGguccggg (SEQ ID NO: 1993), GAGguccggu (SEQ ID NO: 1994), GAGguccuug (SEQ ID NO: 1995), GAGgucgggg (SEQ ID NO: 1996), GAGgucucgu (SEQ ID NO: 1997), GAGgucugag (SEQ ID NO: 1998), GAGgucuggu (SEQ ID NO: 1999), GAGgucuguc (SEQ ID NO: 2000), GAGgucuguu (SEQ ID NO: 2001), GAGgucuuuu (SEQ ID NO: 2002), GAGgugaaaa (SEQ ID NO: 2003), GAGgugaaau (SEQ ID NO: 2004), GAGgugaaca (SEQ ID NO: 2005), GAGgugaagg (SEQ ID NO: 2006), GAGgugaaua (SEQ ID NO: 2007), GAGgugaauu (SEQ ID NO: 2008), GAGgugacau (SEQ ID NO: 2009), GAGgugacca (SEQ ID NO: 2010), GAGgugaccu (SEQ ID NO: 2011), GAGgugacua (SEQ ID NO: 2012), GAGgugacuu (SEQ ID NO: 2013), GAGgugagaa (SEQ ID NO: 2014), GAGgugagac (SEQ ID NO: 2015), GAGgugagag (SEQ ID NO: 2016), GAGgugagau (SEQ ID NO: 2017), GAGgugagca (SEQ ID NO: 2018), GAGgugagcc (SEQ ID NO: 2019), GAGgugagcg (SEQ ID NO: 2020), GAGgugagcu (SEQ ID NO: 2021), GAGgugagga (SEQ ID NO: 2022), GAGgugaggc (SEQ ID NO: 2023), GAGgugaggg (SEQ ID NO: 2024), GAGgugagua (SEQ ID NO: 2025), GAGgugagug (SEQ ID NO: 2026), GAGgugaguu (SEQ ID NO: 2027), GAGgugauau (SEQ ID NO: 2028), GAGgugaucc (SEQ ID NO: 2029), GAGgugaucu (SEQ ID NO: 2030), GAGgugauga (SEQ ID NO: 2031), GAGgugaugg (SEQ ID NO: 2032), GAGgugaugu (SEQ ID NO: 2033), GAGgugauuc (SEQ ID NO: 2034), GAGgugcaca (SEQ ID NO: 2035), GAGgugcaga (SEQ ID NO: 2036), GAGgugcagc (SEQ ID NO: 2037), GAGgugcagg (SEQ ID NO: 2038), GAGgugccag (SEQ ID NO: 2039), GAGgugccca (SEQ ID NO: 2040), GAGgugccuu (SEQ ID NO: 2041), GAGgugcggg (SEQ ID NO: 2042), GAGgugcgug (SEQ ID NO: 2043), GAGgugcucc (SEQ ID NO: 2044), GAGgugcugg (SEQ ID NO: 2045), GAGgugcuua (SEQ ID NO: 2046), GAGgugcuug (SEQ ID NO: 2047), GAGguggaaa (SEQ ID NO: 2048), GAGguggaau (SEQ ID NO: 2049), GAGguggacc (SEQ ID NO: 2050), GAGguggacg (SEQ ID NO: 2051), GAGguggagg (SEQ ID NO: 2052), GAGguggcug (SEQ ID NO: 2053), GAGgugggaa (SEQ ID NO: 2054), GAGgugggag (SEQ ID NO: 2055), GAGgugggau (SEQ ID NO: 2056), GAGgugggca (SEQ ID NO: 2057), GAGgugggcg (SEQ ID NO: 2058), GAGgugggcu (SEQ ID NO: 2059), GAGgugggga (SEQ ID NO: 2060), GAGguggggc (SEQ ID NO: 2061), GAGguggggg (SEQ ID NO: 2062), GAGgugggua (SEQ ID NO: 2063), GAGguggguc (SEQ ID NO: 2064), GAGgugggug (SEQ ID NO: 2065), GAGguggguu (SEQ ID NO: 2066), GAGgugguau (SEQ ID NO: 2067), GAGgugguuc (SEQ ID NO: 2068), GAGgugucau (SEQ ID NO: 2069), GAGgugugag (SEQ ID NO: 2070), GAGgugugau (SEQ ID NO: 2071), GAGgugugca (SEQ ID NO: 2072), GAGgugugcu (SEQ ID NO: 2073), GAGgugugga (SEQ ID NO: 2074), GAGguguggg (SEQ ID NO: 2075), GAGguguggu (SEQ ID NO: 2076), GAGgugugua (SEQ ID NO: 2077), GAGgugugug (SEQ ID NO: 2078), GAGguuaaau (SEQ ID NO: 2079), GAGguuaaga (SEQ ID NO: 2080), GAGguuaaua (SEQ ID NO: 2081), GAGguuaccg (SEQ ID NO: 2082), GAGguuagaa (SEQ ID NO: 2083), GAGguuagac (SEQ ID NO: 2084), GAGguuagag (SEQ ID NO: 2085), GAGguuaggu (SEQ ID NO: 2086), GAGguuagua (SEQ ID NO: 2087), GAGguuaguc (SEQ ID NO: 2088), GAGguuagug (SEQ ID NO: 2089), GAGguuaguu (SEQ ID NO: 2090), GAGguuaugu (SEQ ID NO: 2091), GAGguuauuc (SEQ ID NO: 2092), GAGguucaaa (SEQ ID NO: 2093), GAGguucaua (SEQ ID NO: 2094), GAGguucuga (SEQ ID NO: 2095), GAGguugaag (SEQ ID NO: 2096), GAGguugcag (SEQ ID NO: 2097), GAGguugcug (SEQ ID NO: 2098), GAGguuggaa (SEQ ID NO: 2099), GAGguuggag (SEQ ID NO: 2100), GAGguuggau (SEQ ID NO: 2101), GAGguuggua (SEQ ID NO: 2102), GAGguugguc (SEQ ID NO: 2103), GAGguugguu (SEQ ID NO: 2104), GAGguuguag (SEQ ID NO: 2105), GAGguuucug (SEQ ID NO: 2106), GAGguuugag (SEQ ID NO: 2107), GAGguuugga (SEQ ID NO: 2108), GAGguuuggg (SEQ ID NO: 2109), GAGguuugua (SEQ ID NO: 2110), GAGguuuguu (SEQ ID NO: 2111), GAGguuuuca (SEQ ID NO: 2112), GAGguuuuga (SEQ ID NO: 2113), GAGguuuugg (SEQ ID NO: 2114), GAGguuuuua (SEQ ID NO: 2115), GAGguuuuuc (SEQ ID NO: 2116), GAUguaaaau (SEQ ID NO: 2117), GAUguaagca (SEQ ID NO: 2118), GAUguaagcc (SEQ ID NO: 2119), GAUguaaggu (SEQ ID NO: 2120), GAUguaagua (SEQ ID NO: 2121), GAUguaagug (SEQ ID NO: 2122), GAUguaaguu (SEQ ID NO: 2123), GAUguacauc (SEQ ID NO: 2124), GAUguaggua (SEQ ID NO: 2125), GAUguauggc (SEQ ID NO: 2126), GAUguaugua (SEQ ID NO: 2127), GAUguauguu (SEQ ID NO: 2128), GAUgucagug (SEQ ID NO: 2129), GAUgugagag (SEQ ID NO: 2130), GAUgugagcc (SEQ ID NO: 2131), GAUgugagcu (SEQ ID NO: 2132), GAUgugagga (SEQ ID NO: 2133), GAUgugaguc (SEQ ID NO: 2134), GAUgugagug (SEQ ID NO: 2135), GAUgugaguu (SEQ ID NO: 2136), GAUgugggua (SEQ ID NO: 2137), GAUgugggug (SEQ ID NO: 2138), GAUguguguu (SEQ ID NO: 2139), GAUguuagcu (SEQ ID NO: 2140), GAUguucagu (SEQ ID NO: 2141), GAUguucgug (SEQ ID NO: 2142), GAUguuuguu (SEQ ID NO: 2143), GCAguaaagg (SEQ ID NO: 2144), GCAguaagaa (SEQ ID NO: 2145), GCAguaagga (SEQ ID NO: 2146), GCAguaagua (SEQ ID NO: 2147), GCAguaaguc (SEQ ID NO: 2148), GCAguaaguu (SEQ ID NO: 2149), GCAguagaug (SEQ ID NO: 2150), GCAguaggua (SEQ ID NO: 2151), GCAguaugug (SEQ ID NO: 2152), GCAguauguu (SEQ ID NO: 2153), GCAgucagua (SEQ ID NO: 2154), GCAgucagug (SEQ ID NO: 2155), GCAguccggu (SEQ ID NO: 2156), GCAgugacuu (SEQ ID NO: 2157), GCAgugagcc (SEQ ID NO: 2158), GCAgugagcg (SEQ ID NO: 2159), GCAgugagcu (SEQ ID NO: 2160), GCAgugagua (SEQ ID NO: 2161), GCAgugagug (SEQ ID NO: 2162), GCAgugaguu (SEQ ID NO: 2163), GCAgugggua (SEQ ID NO: 2164), GCAguuaagu (SEQ ID NO: 2165), GCAguugagu (SEQ ID NO: 2166), GCCguaaguc (SEQ ID NO: 2167), GCCgugagua (SEQ ID NO: 2168), GCGguaaagc (SEQ ID NO: 2169), GCGguaaaua (SEQ ID NO: 2170), GCGguaagcu (SEQ ID NO: 2171), GCGguaaggg (SEQ ID NO: 2172), GCGguaagug (SEQ ID NO: 2173), GCGguaauca (SEQ ID NO: 2174), GCGguacgua (SEQ ID NO: 2175), GCGguacuug (SEQ ID NO: 2176), GCGguagggu (SEQ ID NO: 2177), GCGguagugu (SEQ ID NO: 2178), GCGgugagca (SEQ ID NO: 2179), GCGgugagcu (SEQ ID NO: 2180), GCGgugaguu (SEQ ID NO: 2181), GCGguggcuc (SEQ ID NO: 2182), GCGgugugca (SEQ ID NO: 2183), GCGguguguu (SEQ ID NO: 2184), GCGguuaagu (SEQ ID NO: 2185), GCGguuugca (SEQ ID NO: 2186), GCUgcuguaa (SEQ ID NO: 2187), GCUguaaaua (SEQ ID NO: 2188), GCUguaagac (SEQ ID NO: 2189), GCUguaagag (SEQ ID NO: 2190), GCUguaagca (SEQ ID NO: 2191), GCUguaagga (SEQ ID NO: 2192), GCUguaagua (SEQ ID NO: 2193), GCUguaaguc (SEQ ID NO: 2194), GCUguaagug (SEQ ID NO: 2195), GCUguaaguu (SEQ ID NO: 2196), GCUguaggug (SEQ ID NO: 2197), GCUguauggu (SEQ ID NO: 2198), GCUgucagug (SEQ ID NO: 2199), GCUguccuug (SEQ ID NO: 2200), GCUgugagaa (SEQ ID NO: 2201), GCUgugagcc (SEQ ID NO: 2202), GCUgugagga (SEQ ID NO: 2203), GCUgugagua (SEQ ID NO: 2204), GCUgugaguc (SEQ ID NO: 2205), GCUgugagug (SEQ ID NO: 2206), GCUgugaguu (SEQ ID NO: 2207), GCUguggguu (SEQ ID NO: 2208), GGAguaagag (SEQ ID NO: 2209), GGAguaagca (SEQ ID NO: 2210), GGAguaagcc (SEQ ID NO: 2211), GGAguaagcu (SEQ ID NO: 2212), GGAguaagga (SEQ ID NO: 2213), GGAguaagug (SEQ ID NO: 2214), GGAguaaguu (SEQ ID NO: 2215), GGAguaauuu (SEQ ID NO: 2216), GGAguacugu (SEQ ID NO: 2217), GGAguaggaa (SEQ ID NO: 2218), GGAguaggua (SEQ ID NO: 2219), GGAguagguu (SEQ ID NO: 2220), GGAguaguau (SEQ ID NO: 2221), GGAguaugac (SEQ ID NO: 2222), GGAguauggu (SEQ ID NO: 2223), GGAgucaagu (SEQ ID NO: 2224), GGAgugaggg (SEQ ID NO: 2225), GGAgugagua (SEQ ID NO: 2226), GGAgugaguc (SEQ ID NO: 2227), GGAgugagug (SEQ ID NO: 2228), GGAgugaguu (SEQ ID NO: 2229), GGAgugcuuu (SEQ ID NO: 2230), GGAgugggca (SEQ ID NO: 2231), GGAgugggug (SEQ ID NO: 2232), GGAguuaagg (SEQ ID NO: 2233), GGAguugaga (SEQ ID NO: 2234), GGCguaagcc (SEQ ID NO: 2235), GGCguaggua (SEQ ID NO: 2236), GGCguaggug (SEQ ID NO: 2237), GGCgugagcc (SEQ ID NO: 2238), GGCgugaguc (SEQ ID NO: 2239), GGGguaaaca (SEQ ID NO: 2240), GGGguaaacc (SEQ ID NO: 2241), GGGguaaacu (SEQ ID NO: 2242), GGGguaagaa (SEQ ID NO: 2243), GGGguaagag (SEQ ID NO: 2244), GGGguaagau (SEQ ID NO: 2245), GGGguaagca (SEQ ID NO: 2246), GGGguaagcc (SEQ ID NO: 2247), GGGguaagcu (SEQ ID NO: 2248), GGGguaagga (SEQ ID NO: 2249), GGGguaaggg (SEQ ID NO: 2250), GGGguaagua (SEQ ID NO: 2251), GGGguaagug (SEQ ID NO: 2252), GGGguaaguu (SEQ ID NO: 2253), GGGguagaca (SEQ ID NO: 2254), GGGguaggag (SEQ ID NO: 2255), GGGguaggcc (SEQ ID NO: 2256), GGGguaggga (SEQ ID NO: 2257), GGGguaggua (SEQ ID NO: 2258), GGGguaggug (SEQ ID NO: 2259), GGGguagguu (SEQ ID NO: 2260), GGGguagugc (SEQ ID NO: 2261), GGGguaucug (SEQ ID NO: 2262), GGGguaugac (SEQ ID NO: 2263), GGGguaugga (SEQ ID NO: 2264), GGGguaugua (SEQ ID NO: 2265), GGGguauguc (SEQ ID NO: 2266), GGGguaugug (SEQ ID NO: 2267), GGGguauguu (SEQ ID NO: 2268), GGGgucagua (SEQ ID NO: 2269), GGGguccgug (SEQ ID NO: 2270), GGGgucggag (SEQ ID NO: 2271), GGGgucugug (SEQ ID NO: 2272), GGGgugaaca (SEQ ID NO: 2273), GGGgugaaga (SEQ ID NO: 2274), GGGgugagaa (SEQ ID NO: 2275), GGGgugagau (SEQ ID NO: 2276), GGGgugagcc (SEQ ID NO: 2277), GGGgugagcg (SEQ ID NO: 2278), GGGgugagcu (SEQ ID NO: 2279), GGGgugagga (SEQ ID NO: 2280), GGGgugaggc (SEQ ID NO: 2281), GGGgugaggg (SEQ ID NO: 2282), GGGgugaguc (SEQ ID NO: 2283), GGGgugagug (SEQ ID NO: 2284), GGGgugaguu (SEQ ID NO: 2285), GGGgugcgua (SEQ ID NO: 2286), GGGguggggu (SEQ ID NO: 2287), GGGgugggua (SEQ ID NO: 2288), GGGgugggug (SEQ ID NO: 2289), GGGguggguu (SEQ ID NO: 2290), GGGgugugcg (SEQ ID NO: 2291), GGGgugugua (SEQ ID NO: 2292), GGGguguguc (SEQ ID NO: 2293), GGGgugugug (SEQ ID NO: 2294), GGGguuacag (SEQ ID NO: 2295), GGGguuggac (SEQ ID NO: 2296), GGGguuggga (SEQ ID NO: 2297), GGGguuugcc (SEQ ID NO: 2298), GGGguuugua (SEQ ID NO: 2299), GGUguaagaa (SEQ ID NO: 2300), GGUguaagau (SEQ ID NO: 2301), GGUguaagca (SEQ ID NO: 2302), GGUguaagcc (SEQ ID NO: 2303), GGUguaagcg (SEQ ID NO: 2304), GGUguaaguc (SEQ ID NO: 2305), GGUguaagug (SEQ ID NO: 2306), GGUguagguc (SEQ ID NO: 2307), GGUguaggug (SEQ ID NO: 2308), GGUguagguu (SEQ ID NO: 2309), GGUguccgua (SEQ ID NO: 2310), GGUgugagag (SEQ ID NO: 2311), GGUgugagcc (SEQ ID NO: 2312), GGUgugagcu (SEQ ID NO: 2313), GGUgugagua (SEQ ID NO: 2314), GGUgugaguc (SEQ ID NO: 2315), GGUgugcuuc (SEQ ID NO: 2316), GGUguggcug (SEQ ID NO: 2317), GGUgugguga (SEQ ID NO: 2318), GGUgugucug (SEQ ID NO: 2319), GGUguugaaa (SEQ ID NO: 2320), GGUguugcug (SEQ ID NO: 2321), GUAguaagau (SEQ ID NO: 2322), GUAguaagua (SEQ ID NO: 2323), GUAguaagug (SEQ ID NO: 2324), GUAguagcuu (SEQ ID NO: 2325), GUAguaggua (SEQ ID NO: 2326), GUAgucagua (SEQ ID NO: 2327), GUAgugagua (SEQ ID NO: 2328), GUAguggugg (SEQ ID NO: 2329), GUAguuaagu (SEQ ID NO: 2330), GUAguuucug (SEQ ID NO: 2331), GUCguaagug (SEQ ID NO: 2332), GUCgugagug (SEQ ID NO: 2333), GUCgugaguu (SEQ ID NO: 2334), GUGgcaagua (SEQ ID NO: 2335), GUGgcuugua (SEQ ID NO: 2336), GUGguaaaau (SEQ ID NO: 2337), GUGguaaaga (SEQ ID NO: 2338), GUGguaaauu (SEQ ID NO: 2339), GUGguaacau (SEQ ID NO: 2340), GUGguaacua (SEQ ID NO: 2341), GUGguaagaa (SEQ ID NO: 2342), GUGguaagac (SEQ ID NO: 2343), GUGguaagag (SEQ ID NO: 2344), GUGguaagau (SEQ ID NO: 2345), GUGguaagca (SEQ ID NO: 2346), GUGguaagcg (SEQ ID NO: 2347), GUGguaagcu (SEQ ID NO: 2348), GUGguaagga (SEQ ID NO: 2349), GUGguaaggc (SEQ ID NO: 2350), GUGguaagua (SEQ ID NO: 2351), GUGguaaguc (SEQ ID NO: 2352), GUGguaagug (SEQ ID NO: 2353), GUGguaaguu (SEQ ID NO: 2354), GUGguaauga (SEQ ID NO: 2355), GUGguaauuc (SEQ ID NO: 2356), GUGguaauuu (SEQ ID NO: 2357), GUGguacaug (SEQ ID NO: 2358), GUGguacgau (SEQ ID NO: 2359), GUGguacuau (SEQ ID NO: 2360), GUGguacuug (SEQ ID NO: 2361), GUGguagaua (SEQ ID NO: 2362), GUGguagege (SEQ ID NO: 2363), GUGguaggga (SEQ ID NO: 2364), GUGguagguc (SEQ ID NO: 2365), GUGguaggug (SEQ ID NO: 2366), GUGguagguu (SEQ ID NO: 2367), GUGguauaaa (SEQ ID NO: 2368), GUGguaucuc (SEQ ID NO: 2369), GUGguaugaa (SEQ ID NO: 2370), GUGguaugau (SEQ ID NO: 2371), GUGguaugca (SEQ ID NO: 2372), GUGguaugua (SEQ ID NO: 2373), GUGguauguu (SEQ ID NO: 2374), GUGguccgug (SEQ ID NO: 2375), GUGgucuggc (SEQ ID NO: 2376), GUGgugaaac (SEQ ID NO: 2377), GUGgugagaa (SEQ ID NO: 2378), GUGgugagau (SEQ ID NO: 2379), GUGgugagca (SEQ ID NO: 2380), GUGgugagcu (SEQ ID NO: 2381), GUGgugagga (SEQ ID NO: 2382), GUGgugaggc (SEQ ID NO: 2383), GUGgugagug (SEQ ID NO: 2384), GUGgugaguu (SEQ ID NO: 2385), GUGgugauua (SEQ ID NO: 2386), GUGgugauuc (SEQ ID NO: 2387), GUGgugcgau (SEQ ID NO: 2388), GUGgugcuua (SEQ ID NO: 2389), GUGgugggaa (SEQ ID NO: 2390), GUGgugggua (SEQ ID NO: 2391), GUGguggguc (SEQ ID NO: 2392), GUGguguccg (SEQ ID NO: 2393), GUGguuagca (SEQ ID NO: 2394), GUGguuaggu (SEQ ID NO: 2395), GUGguuagug (SEQ ID NO: 2396), GUGguuugca (SEQ ID NO: 2397), GUGguuugua (SEQ ID NO: 2398), GUUguaaggu (SEQ ID NO: 2399), GUUguaagua (SEQ ID NO: 2400), GUUguaaguc (SEQ ID NO: 2401), GUUguaaguu (SEQ ID NO: 2402), GUUguaccac (SEQ ID NO: 2403), GUUguagcgu (SEQ ID NO: 2404), GUUguaugug (SEQ ID NO: 2405), GUUguauguu (SEQ ID NO: 2406), GUUgucugug (SEQ ID NO: 2407), GUUgugagcu (SEQ ID NO: 2408), GUUgugagug (SEQ ID NO: 2409), GUUgugaguu (SEQ ID NO: 2410), GUUgugggua (SEQ ID NO: 2411), GUUguggguu (SEQ ID NO: 2412), UAAguaaaug (SEQ ID NO: 2413), UAAguaacua (SEQ ID NO: 2414), UAAguaagaa (SEQ ID NO: 2415), UAAguaagag (SEQ ID NO: 2416), UAAguaagau (SEQ ID NO: 2417), UAAguaagca (SEQ ID NO: 2418), UAAguaagcu (SEQ ID NO: 2419), UAAguaagga (SEQ ID NO: 2420), UAAguaaggu (SEQ ID NO: 2421), UAAguaagua (SEQ ID NO: 2422), UAAguaaguc (SEQ ID NO: 2423), UAAguaagug (SEQ ID NO: 2424), UAAguaaguu (SEQ ID NO: 2425), UAAguaauaa (SEQ ID NO: 2426), UAAguacuag (SEQ ID NO: 2427), UAAguaguuu (SEQ ID NO: 2428), UAAguauaaa (SEQ ID NO: 2429), UAAguauaca (SEQ ID NO: 2430), UAAguaugua (SEQ ID NO: 2431), UAAguauuau (SEQ ID NO: 2432), UAAguauuuu (SEQ ID NO: 2433), UAAgucuuuu (SEQ ID NO: 2434), UAAgugagac (SEQ ID NO: 2435), UAAgugagga (SEQ ID NO: 2436), UAAgugaggg (SEQ ID NO: 2437), UAAgugagua (SEQ ID NO: 2438), UAAgugaguc (SEQ ID NO: 2439), UAAgugagug (SEQ ID NO: 2440), UAAgugaguu (SEQ ID NO: 2441), UAAgugaucc (SEQ ID NO: 2442), UAAgugauuc (SEQ ID NO: 2443), UAAgugcgug (SEQ ID NO: 2444), UAAguuaagu (SEQ ID NO: 2445), UAAguuccag (SEQ ID NO: 2446), UAAguucuuu (SEQ ID NO: 2447), UAAguuguaa (SEQ ID NO: 2448), UAAguuguau (SEQ ID NO: 2449), UAAguuuguu (SEQ ID NO: 2450), UACguaacug (SEQ ID NO: 2451), UACguaagaa (SEQ ID NO: 2452), UACguaagau (SEQ ID NO: 2453), UACguaagua (SEQ ID NO: 2454), UACguaagug (SEQ ID NO: 2455), UACguauccu (SEQ ID NO: 2456), UACgucuggc (SEQ ID NO: 2457), UACgugacca (SEQ ID NO: 2458), UAGgcaagac (SEQ ID NO: 2459), UAGgcaaguc (SEQ ID NO: 2460), UAGgcagguc (SEQ ID NO: 2461), UAGgcgugug (SEQ ID NO: 2462), UAGguaaaaa (SEQ ID NO: 2463), UAGguaaaac (SEQ ID NO: 2464), UAGguaaaag (SEQ ID NO: 2465), UAGguaaaau (SEQ ID NO: 2466), UAGguaaaca (SEQ ID NO: 2467), UAGguaaaga (SEQ ID NO: 2468), UAGguaaaua (SEQ ID NO: 2469), UAGguaaauc (SEQ ID NO: 2470), UAGguaaaug (SEQ ID NO: 2471), UAGguaaauu (SEQ ID NO: 2472), UAGguaacac (SEQ ID NO: 2473), UAGguaacag (SEQ ID NO: 2474), UAGguaacau (SEQ ID NO: 2475), UAGguaacca (SEQ ID NO: 2476), UAGguaacgg (SEQ ID NO: 2477), UAGguaacua (SEQ ID NO: 2478), UAGguaacuc (SEQ ID NO: 2479), UAGguaacug (SEQ ID NO: 2480), UAGguaacuu (SEQ ID NO: 2481), UAGguaagac (SEQ ID NO: 2482), UAGguaagag (SEQ ID NO: 2483), UAGguaagau (SEQ ID NO: 2484), UAGguaagca (SEQ ID NO: 2485), UAGguaagcc (SEQ ID NO: 2486), UAGguaagcu (SEQ ID NO: 2487), UAGguaagga (SEQ ID NO: 2488), UAGguaaggc (SEQ ID NO: 2489), UAGguaaggg (SEQ ID NO: 2490), UAGguaagua (SEQ ID NO: 2491), UAGguaaguc (SEQ ID NO: 2492), UAGguaagug (SEQ ID NO: 2493), UAGguaaguu (SEQ ID NO: 2494), UAGguaauag (SEQ ID NO: 2495), UAGguaauau (SEQ ID NO: 2496), UAGguaaucu (SEQ ID NO: 2497), UAGguaauga (SEQ ID NO: 2498), UAGguaaugg (SEQ ID NO: 2499), UAGguaaugu (SEQ ID NO: 2500), UAGguaauua (SEQ ID NO: 2501), UAGguaauuc (SEQ ID NO: 2502), UAGguaauuu (SEQ ID NO: 2503), UAGguacagc (SEQ ID NO: 2504), UAGguacagu (SEQ ID NO: 2505), UAGguacauu (SEQ ID NO: 2506), UAGguaccag (SEQ ID NO: 2507), UAGguaccua (SEQ ID NO: 2508), UAGguaccuu (SEQ ID NO: 2509), UAGguacgag (SEQ ID NO: 2510), UAGguacgua (SEQ ID NO: 2511), UAGguacguu (SEQ ID NO: 2512), UAGguacuau (SEQ ID NO: 2513), UAGguacuga (SEQ ID NO: 2514), UAGguacugg (SEQ ID NO: 2515), UAGguacuuc (SEQ ID NO: 2516), UAGguacuuu (SEQ ID NO: 2517), UAGguagcgg (SEQ ID NO: 2518), UAGguaggaa (SEQ ID NO: 2519), UAGguaggac (SEQ ID NO: 2520), UAGguaggau (SEQ ID NO: 2521), UAGguaggga (SEQ ID NO: 2522), UAGguagggg (SEQ ID NO: 2523), UAGguaggua (SEQ ID NO: 2524), UAGguagguc (SEQ ID NO: 2525), UAGguaggug (SEQ ID NO: 2526), UAGguagguu (SEQ ID NO: 2527), UAGguaguaa (SEQ ID NO: 2528), UAGguagucu (SEQ ID NO: 2529), UAGguagugg (SEQ ID NO: 2530), UAGguagugu (SEQ ID NO: 2531), UAGguaguuu (SEQ ID NO: 2532), UAGguauaaa (SEQ ID NO: 2533), UAGguauaac (SEQ ID NO: 2534), UAGguauaag (SEQ ID NO: 2535), UAGguauaau (SEQ ID NO: 2536), UAGguauaca (SEQ ID NO: 2537), UAGguauacu (SEQ ID NO: 2538), UAGguauaua (SEQ ID NO: 2539), UAGguauauc (SEQ ID NO: 2540), UAGguauauu (SEQ ID NO: 2541), UAGguaucag (SEQ ID NO: 2542), UAGguaucua (SEQ ID NO: 2543), UAGguaucuc (SEQ ID NO: 2544), UAGguaugaa (SEQ ID NO: 2545), UAGguaugag (SEQ ID NO: 2546), UAGguaugca (SEQ ID NO: 2547), UAGguaugga (SEQ ID NO: 2548), UAGguauggc (SEQ ID NO: 2549), UAGguauggu (SEQ ID NO: 2550), UAGguaugua (SEQ ID NO: 2551), UAGguauguc (SEQ ID NO: 2552), UAGguaugug (SEQ ID NO: 2553), UAGguauguu (SEQ ID NO: 2554), UAGguauuaa (SEQ ID NO: 2555), UAGguauuac (SEQ ID NO: 2556), UAGguauuau (SEQ ID NO: 2557), UAGguauuca (SEQ ID NO: 2558), UAGguauucc (SEQ ID NO: 2559), UAGguauucu (SEQ ID NO: 2560), UAGguauuga (SEQ ID NO: 2561), UAGguauuua (SEQ ID NO: 2562), UAGguauuuc (SEQ ID NO: 2563), UAGguauuuu (SEQ ID NO: 2564), UAGgucacuc (SEQ ID NO: 2565), UAGgucagcu (SEQ ID NO: 2566), UAGgucaggu (SEQ ID NO: 2567), UAGgucagua (SEQ ID NO: 2568), UAGgucagug (SEQ ID NO: 2569), UAGgucaguu (SEQ ID NO: 2570), UAGgucaucu (SEQ ID NO: 2571), UAGgucauug (SEQ ID NO: 2572), UAGguccaau (SEQ ID NO: 2573), UAGguccugu (SEQ ID NO: 2574), UAGgucucaa (SEQ ID NO: 2575), UAGgucucgc (SEQ ID NO: 2576), UAGgucuggc (SEQ ID NO: 2577), UAGgucuguc (SEQ ID NO: 2578), UAGgucugug (SEQ ID NO: 2579), UAGgugaagu (SEQ ID NO: 2580), UAGgugaaua (SEQ ID NO: 2581), UAGgugaaug (SEQ ID NO: 2582), UAGgugaauu (SEQ ID NO: 2583), UAGgugacau (SEQ ID NO: 2584), UAGgugacca (SEQ ID NO: 2585), UAGgugacua (SEQ ID NO: 2586), UAGgugagaa (SEQ ID NO: 2587), UAGgugagac (SEQ ID NO: 2588), UAGgugagag (SEQ ID NO: 2589), UAGgugagau (SEQ ID NO: 2590), UAGgugagcc (SEQ ID NO: 2591), UAGgugagcu (SEQ ID NO: 2592), UAGgugagga (SEQ ID NO: 2593), UAGgugaggc (SEQ ID NO: 2594), UAGgugaggu (SEQ ID NO: 2595), UAGgugagua (SEQ ID NO: 2596), UAGgugaguc (SEQ ID NO: 2597), UAGgugagug (SEQ ID NO: 2598), UAGgugauca (SEQ ID NO: 2599), UAGgugauuc (SEQ ID NO: 2600), UAGgugauuu (SEQ ID NO: 2601), UAGgugcaua (SEQ ID NO: 2602), UAGgugcauc (SEQ ID NO: 2603), UAGgugccgu (SEQ ID NO: 2604), UAGgugccug (SEQ ID NO: 2605), UAGgugcgca (SEQ ID NO: 2606), UAGgugcgua (SEQ ID NO: 2607), UAGgugcgug (SEQ ID NO: 2608), UAGgugcuga (SEQ ID NO: 2609), UAGguggaua (SEQ ID NO: 2610), UAGgugggaa (SEQ ID NO: 2611), UAGgugggac (SEQ ID NO: 2612), UAGgugggag (SEQ ID NO: 2613), UAGgugggau (SEQ ID NO: 2614), UAGgugggcc (SEQ ID NO: 2615), UAGgugggcu (SEQ ID NO: 2616), UAGguggguu (SEQ ID NO: 2617), UAGguggugu (SEQ ID NO: 2618), UAGguguaaa (SEQ ID NO: 2619), UAGgugugaa (SEQ ID NO: 2620), UAGgugugag (SEQ ID NO: 2621), UAGgugugca (SEQ ID NO: 2622), UAGgugugcc (SEQ ID NO: 2623), UAGgugugcg (SEQ ID NO: 2624), UAGguguggu (SEQ ID NO: 2625), UAGgugugua (SEQ ID NO: 2626), UAGgugugug (SEQ ID NO: 2627), UAGguguugg (SEQ ID NO: 2628), UAGguuaagc (SEQ ID NO: 2629), UAGguuagac (SEQ ID NO: 2630), UAGguuagcc (SEQ ID NO: 2631), UAGguuaggc (SEQ ID NO: 2632), UAGguuagua (SEQ ID NO: 2633), UAGguuaguc (SEQ ID NO: 2634), UAGguuagug (SEQ ID NO: 2635), UAGguucccc (SEQ ID NO: 2636), UAGguucuac (SEQ ID NO: 2637), UAGguuggua (SEQ ID NO: 2638), UAGguugguu (SEQ ID NO: 2639), UAGguugucc (SEQ ID NO: 2640), UAGguuuauu (SEQ ID NO: 2641), UAGguuugcc (SEQ ID NO: 2642), UAGguuugua (SEQ ID NO: 2643), UAGguuuguc (SEQ ID NO: 2644), UAGguuugug (SEQ ID NO: 2645), UAGguuuguu (SEQ ID NO: 2646), UAGguuuuuc (SEQ ID NO: 2647), UAGguuuuug (SEQ ID NO: 2648), UAUguaagaa (SEQ ID NO: 2649), UAUguaagau (SEQ ID NO: 2650), UAUguaagca (SEQ ID NO: 2651), UAUguaagcc (SEQ ID NO: 2652), UAUguaagua (SEQ ID NO: 2653), UAUguaaguc (SEQ ID NO: 2654), UAUguaagug (SEQ ID NO: 2655), UAUguaaguu (SEQ ID NO: 2656), UAUguacgug (SEQ ID NO: 2657), UAUguacguu (SEQ ID NO: 2658), UAUguagguc (SEQ ID NO: 2659), UAUguagguu (SEQ ID NO: 2660), UAUguauccu (SEQ ID NO: 2661), UAUguaucuc (SEQ ID NO: 2662), UAUguaugua (SEQ ID NO: 2663), UAUguauguc (SEQ ID NO: 2664), UAUguaugug (SEQ ID NO: 2665), UAUguauuau (SEQ ID NO: 2666), UAUgucagaa (SEQ ID NO: 2667), UAUgucugua (SEQ ID NO: 2668), UAUgugaaua (SEQ ID NO: 2669), UAUgugacag (SEQ ID NO: 2670), UAUgugagua (SEQ ID NO: 2671), UAUgugagug (SEQ ID NO: 2672), UAUgugaguu (SEQ ID NO: 2673), UAUgugggca (SEQ ID NO: 2674), UAUgugugua (SEQ ID NO: 2675), UAUguguuua (SEQ ID NO: 2676), UAUguuuugu (SEQ ID NO: 2677), UCAgcgacau (SEQ ID NO: 2678), UCAguaaaau (SEQ ID NO: 2679), UCAguaaaua (SEQ ID NO: 2680), UCAguaacug (SEQ ID NO: 2681), UCAguaagaa (SEQ ID NO: 2682), UCAguaagag (SEQ ID NO: 2683), UCAguaagau (SEQ ID NO: 2684), UCAguaagca (SEQ ID NO: 2685), UCAguaagcc (SEQ ID NO: 2686), UCAguaagcu (SEQ ID NO: 2687), UCAguaaggg (SEQ ID NO: 2688), UCAguaagua (SEQ ID NO: 2689), UCAguaaguc (SEQ ID NO: 2690), UCAguaagug (SEQ ID NO: 2691), UCAguaaguu (SEQ ID NO: 2692), UCAguaucuu (SEQ ID NO: 2693), UCAguaugga (SEQ ID NO: 2694), UCAguauggu (SEQ ID NO: 2695), UCAgucccca (SEQ ID NO: 2696), UCAgugagca (SEQ ID NO: 2697), UCAgugagcu (SEQ ID NO: 2698), UCAgugagua (SEQ ID NO: 2699), UCAgugagug (SEQ ID NO: 2700), UCAgugaguu (SEQ ID NO: 2701), UCAgugauug (SEQ ID NO: 2702), UCAgugggug (SEQ ID NO: 2703), UCAguugagc (SEQ ID NO: 2704), UCAguugauu (SEQ ID NO: 2705), UCAguuuagu (SEQ ID NO: 2706), UCCguaagca (SEQ ID NO: 2707), UCCguaagcu (SEQ ID NO: 2708), UCCguaaguc (SEQ ID NO: 2709), UCCguaagug (SEQ ID NO: 2710), UCCguaauag (SEQ ID NO: 2711), UCCguacuua (SEQ ID NO: 2712), UCCguaugua (SEQ ID NO: 2713), UCCguauguu (SEQ ID NO: 2714), UCCgugagau (SEQ ID NO: 2715), UCCgugaguc (SEQ ID NO: 2716), UCGguaaauu (SEQ ID NO: 2717), UCGguaagag (SEQ ID NO: 2718), UCGguaagcu (SEQ ID NO: 2719), UCGguacauc (SEQ ID NO: 2720), UCGguacucc (SEQ ID NO: 2721), UCGguagacc (SEQ ID NO: 2722), UCGguagguu (SEQ ID NO: 2723), UCGguaguaa (SEQ ID NO: 2724), UCGguaugug (SEQ ID NO: 2725), UCGguauguu (SEQ ID NO: 2726), UCGguauuga (SEQ ID NO: 2727), UCGgucagua (SEQ ID NO: 2728), UCGgucuuag (SEQ ID NO: 2729), UCGgugaagu (SEQ ID NO: 2730), UCGgugagaa (SEQ ID NO: 2731), UCGgugagca (SEQ ID NO: 2732), UCGgugaggc (SEQ ID NO: 2733), UCGgugagua (SEQ ID NO: 2734), UCGgugcgcu (SEQ ID NO: 2735), UCGgugcuuu (SEQ ID NO: 2736), UCGgugguuu (SEQ ID NO: 2737), UCGguuagcu (SEQ ID NO: 2738), UCUguaaaag (SEQ ID NO: 2739), UCUguaagaa (SEQ ID NO: 2740), UCUguaagau (SEQ ID NO: 2741), UCUguaagca (SEQ ID NO: 2742), UCUguaagcu (SEQ ID NO: 2743), UCUguaagua (SEQ ID NO: 2744), UCUguaaguc (SEQ ID NO: 2745), UCUguaagug (SEQ ID NO: 2746), UCUguaaguu (SEQ ID NO: 2747), UCUguaauaa (SEQ ID NO: 2748), UCUguaauga (SEQ ID NO: 2749), UCUguaaugu (SEQ ID NO: 2750), UCUguaggua (SEQ ID NO: 2751), UCUguagguu (SEQ ID NO: 2752), UCUguauaua (SEQ ID NO: 2753), UCUguaugac (SEQ ID NO: 2754), UCUguaugua (SEQ ID NO: 2755), UCUguccueg (SEQ ID NO: 2756), UCUgugagag (SEQ ID NO: 2757), UCUgugagcu (SEQ ID NO: 2758), UCUgugagga (SEQ ID NO: 2759), UCUgugagua (SEQ ID NO: 2760), UCUgugaguc (SEQ ID NO: 2761), UCUgugagug (SEQ ID NO: 2762), UCUgugaguu (SEQ ID NO: 2763), UCUgugcgua (SEQ ID NO: 2764), UCUgugugag (SEQ ID NO: 2765), UGAguaacuu (SEQ ID NO: 2766), UGAguaagau (SEQ ID NO: 2767), UGAguaagca (SEQ ID NO: 2768), UGAguaagcu (SEQ ID NO: 2769), UGAguaaggc (SEQ ID NO: 2770), UGAguaaggu (SEQ ID NO: 2771), UGAguaagua (SEQ ID NO: 2772), UGAguaaguc (SEQ ID NO: 2773), UGAguaagug (SEQ ID NO: 2774), UGAguaaguu (SEQ ID NO: 2775), UGAguaaucc (SEQ ID NO: 2776), UGAguaauua (SEQ ID NO: 2777), UGAguacagu (SEQ ID NO: 2778), UGAguacgua (SEQ ID NO: 2779), UGAguacguu (SEQ ID NO: 2780), UGAguacugu (SEQ ID NO: 2781), UGAguagcug (SEQ ID NO: 2782), UGAguaggua (SEQ ID NO: 2783), UGAguauaaa (SEQ ID NO: 2784), UGAguaugcu (SEQ ID NO: 2785), UGAguaugga (SEQ ID NO: 2786), UGAguaugua (SEQ ID NO: 2787), UGAguauguc (SEQ ID NO: 2788), UGAguauguu (SEQ ID NO: 2789), UGAgucagag (SEQ ID NO: 2790), UGAgucuacg (SEQ ID NO: 2791), UGAgugaaua (SEQ ID NO: 2792), UGAgugaauu (SEQ ID NO: 2793), UGAgugagaa (SEQ ID NO: 2794), UGAgugagau (SEQ ID NO: 2795), UGAgugagca (SEQ ID NO: 2796), UGAgugagcc (SEQ ID NO: 2797), UGAgugagga (SEQ ID NO: 2798), UGAgugagua (SEQ ID NO: 2799), UGAgugagug (SEQ ID NO: 2800), UGAgugaguu (SEQ ID NO: 2801), UGAgugggaa (SEQ ID NO: 2802), UGAguuaaga (SEQ ID NO: 2803), UGAguuaaug (SEQ ID NO: 2804), UGAguuacgg (SEQ ID NO: 2805), UGAguuaggu (SEQ ID NO: 2806), UGAguucuau (SEQ ID NO: 2807), UGAguugguu (SEQ ID NO: 2808), UGAguuguag (SEQ ID NO: 2809), UGAguuuauc (SEQ ID NO: 2810), UGCguaaguc (SEQ ID NO: 2811), UGCguaagug (SEQ ID NO: 2812), UGCguacggc (SEQ ID NO: 2813), UGCguacggg (SEQ ID NO: 2814), UGCguaugua (SEQ ID NO: 2815), UGGgcaaguc (SEQ ID NO: 2816), UGGgcaagug (SEQ ID NO: 2817), UGGgcacauc (SEQ ID NO: 2818), UGGgccacgu (SEQ ID NO: 2819), UGGgccccgg (SEQ ID NO: 2820), UGGguaaaau (SEQ ID NO: 2821), UGGguaaagc (SEQ ID NO: 2822), UGGguaaagg (SEQ ID NO: 2823), UGGguaaagu (SEQ ID NO: 2824), UGGguaaaua (SEQ ID NO: 2825), UGGguaaaug (SEQ ID NO: 2826), UGGguaaauu (SEQ ID NO: 2827), UGGguaacag (SEQ ID NO: 2828), UGGguaacau (SEQ ID NO: 2829), UGGguaacua (SEQ ID NO: 2830), UGGguaacuu (SEQ ID NO: 2831), UGGguaagaa (SEQ ID NO: 2832), UGGguaagac (SEQ ID NO: 2833), UGGguaagag (SEQ ID NO: 2834), UGGguaagau (SEQ ID NO: 2835), UGGguaagca (SEQ ID NO: 2836), UGGguaagcc (SEQ ID NO: 2837), UGGguaagcu (SEQ ID NO: 2838), UGGguaaggg (SEQ ID NO: 2839), UGGguaaggu (SEQ ID NO: 2840), UGGguaagua (SEQ ID NO: 2841), UGGguaaguc (SEQ ID NO: 2842), UGGguaagug (SEQ ID NO: 2843), UGGguaaguu (SEQ ID NO: 2844), UGGguaaugu (SEQ ID NO: 2845), UGGguaauua (SEQ ID NO: 2846), UGGguaauuu (SEQ ID NO: 2847), UGGguacaaa (SEQ ID NO: 2848), UGGguacagu (SEQ ID NO: 2849), UGGguacuac (SEQ ID NO: 2850), UGGguaggga (SEQ ID NO: 2851), UGGguagguc (SEQ ID NO: 2852), UGGguaggug (SEQ ID NO: 2853), UGGguagguu (SEQ ID NO: 2854), UGGguaguua (SEQ ID NO: 2855), UGGguauagu (SEQ ID NO: 2856), UGGguaugaa (SEQ ID NO: 2857), UGGguaugac (SEQ ID NO: 2858), UGGguaugag (SEQ ID NO: 2859), UGGguaugua (SEQ ID NO: 2860), UGGguauguc (SEQ ID NO: 2861), UGGguaugug (SEQ ID NO: 2862), UGGguauguu (SEQ ID NO: 2863), UGGguauuug (SEQ ID NO: 2864), UGGgucuuug (SEQ ID NO: 2865), UGGgugaccu (SEQ ID NO: 2866), UGGgugacua (SEQ ID NO: 2867), UGGgugagac (SEQ ID NO: 2868), UGGgugagag (SEQ ID NO: 2869), UGGgugagca (SEQ ID NO: 2870), UGGgugagcc (SEQ ID NO: 2871), UGGgugagga (SEQ ID NO: 2872), UGGgugaggc (SEQ ID NO: 2873), UGGgugaggg (SEQ ID NO: 2874), UGGgugagua (SEQ ID NO: 2875), UGGgugaguc (SEQ ID NO: 2876), UGGgugagug (SEQ ID NO: 2877), UGGgugaguu (SEQ ID NO: 2878), UGGgugcgug (SEQ ID NO: 2879), UGGguggagg (SEQ ID NO: 2880), UGGguggcuu (SEQ ID NO: 2881), UGGguggggg (SEQ ID NO: 2882), UGGgugggua (SEQ ID NO: 2883), UGGguggguc (SEQ ID NO: 2884), UGGgugggug (SEQ ID NO: 2885), UGGguggguu (SEQ ID NO: 2886), UGGgugugga (SEQ ID NO: 2887), UGGguguguc (SEQ ID NO: 2888), UGGgugugug (SEQ ID NO: 2889), UGGguguguu (SEQ ID NO: 2890), UGGguguuua (SEQ ID NO: 2891), UGGguuaaug (SEQ ID NO: 2892), UGGguuaguc (SEQ ID NO: 2893), UGGguuagug (SEQ ID NO: 2894), UGGguuaguu (SEQ ID NO: 2895), UGGguucaag (SEQ ID NO: 2896), UGGguucgua (SEQ ID NO: 2897), UGGguuggug (SEQ ID NO: 2898), UGGguuuaag (SEQ ID NO: 2899), UGGguuugua (SEQ ID NO: 2900), UGUgcaagua (SEQ ID NO: 2901), UGUguaaaua (SEQ ID NO: 2902), UGUguaagaa (SEQ ID NO: 2903), UGUguaagac (SEQ ID NO: 2904), UGUguaagag (SEQ ID NO: 2905), UGUguaaggu (SEQ ID NO: 2906), UGUguaagua (SEQ ID NO: 2907), UGUguaaguc (SEQ ID NO: 2908), UGUguaaguu (SEQ ID NO: 2909), UGUguacuuc (SEQ ID NO: 2910), UGUguaggeg (SEQ ID NO: 2911), UGUguaggua (SEQ ID NO: 2912), UGUguaguua (SEQ ID NO: 2913), UGUguaugug (SEQ ID NO: 2914), UGUgucagua (SEQ ID NO: 2915), UGUgucugua (SEQ ID NO: 2916), UGUgucuguc (SEQ ID NO: 2917), UGUgugaccc (SEQ ID NO: 2918), UGUgugagau (SEQ ID NO: 2919), UGUgugagca (SEQ ID NO: 2920), UGUgugagcc (SEQ ID NO: 2921), UGUgugagua (SEQ ID NO: 2922), UGUgugaguc (SEQ ID NO: 2923), UGUgugagug (SEQ ID NO: 2924), UGUgugcgug (SEQ ID NO: 2925), UGUgugggug (SEQ ID NO: 2926), UGUguggguu (SEQ ID NO: 2927), UGUgugugag (SEQ ID NO: 2928), UGUguguucu (SEQ ID NO: 2929), UGUguuuaga (SEQ ID NO: 2930), UUAguaaaua (SEQ ID NO: 2931), UUAguaagaa (SEQ ID NO: 2932), UUAguaagua (SEQ ID NO: 2933), UUAguaagug (SEQ ID NO: 2934), UUAguaaguu (SEQ ID NO: 2935), UUAguaggug (SEQ ID NO: 2936), UUAgugagca (SEQ ID NO: 2937), UUAgugaguu (SEQ ID NO: 2938), UUAguuaagu (SEQ ID NO: 2939), UUCguaaguc (SEQ ID NO: 2940), UUCguaaguu (SEQ ID NO: 2941), UUCguaauua (SEQ ID NO: 2942), UUCgugagua (SEQ ID NO: 2943), UUCgugaguu (SEQ ID NO: 2944), UUGgcaagug (SEQ ID NO: 2945), UUGgccgagu (SEQ ID NO: 2946), UUGguaaaaa (SEQ ID NO: 2947), UUGguaaaau (SEQ ID NO: 2948), UUGguaaaga (SEQ ID NO: 2949), UUGguaaagg (SEQ ID NO: 2950), UUGguaaagu (SEQ ID NO: 2951), UUGguaaauc (SEQ ID NO: 2952), UUGguaaaug (SEQ ID NO: 2953), UUGguaaauu (SEQ ID NO: 2954), UUGguaacug (SEQ ID NO: 2955), UUGguaacuu (SEQ ID NO: 2956), UUGguaagaa (SEQ ID NO: 2957), UUGguaagag (SEQ ID NO: 2958), UUGguaagcu (SEQ ID NO: 2959), UUGguaagga (SEQ ID NO: 2960), UUGguaaggg (SEQ ID NO: 2961), UUGguaagua (SEQ ID NO: 2962), UUGguaagug (SEQ ID NO: 2963), UUGguaaguu (SEQ ID NO: 2964), UUGguaauac (SEQ ID NO: 2965), UUGguaauca (SEQ ID NO: 2966), UUGguaaugc (SEQ ID NO: 2967), UUGguaaugu (SEQ ID NO: 2968), UUGguaauug (SEQ ID NO: 2969), UUGguaauuu (SEQ ID NO: 2970), UUGguacaua (SEQ ID NO: 2971), UUGguacgug (SEQ ID NO: 2972), UUGguagagg (SEQ ID NO: 2973), UUGguaggac (SEQ ID NO: 2974), UUGguaggcg (SEQ ID NO: 2975), UUGguaggcu (SEQ ID NO: 2976), UUGguaggga (SEQ ID NO: 2977), UUGguaggua (SEQ ID NO: 2978), UUGguagguc (SEQ ID NO: 2979), UUGguaggug (SEQ ID NO: 2980), UUGguauaaa (SEQ ID NO: 2981), UUGguauaca (SEQ ID NO: 2982), UUGguauauu (SEQ ID NO: 2983), UUGguaucua (SEQ ID NO: 2984), UUGguaucuc (SEQ ID NO: 2985), UUGguaugca (SEQ ID NO: 2986), UUGguaugua (SEQ ID NO: 2987), UUGguaugug (SEQ ID NO: 2988), UUGguauguu (SEQ ID NO: 2989), UUGguauugu (SEQ ID NO: 2990), UUGguauuua (SEQ ID NO: 2991), UUGguauuuu (SEQ ID NO: 2992), UUGgucagaa (SEQ ID NO: 2993), UUGgucagua (SEQ ID NO: 2994), UUGgucucug (SEQ ID NO: 2995), UUGgucugca (SEQ ID NO: 2996), UUGgugaaaa (SEQ ID NO: 2997), UUGgugacug (SEQ ID NO: 2998), UUGgugagac (SEQ ID NO: 2999), UUGgugagau (SEQ ID NO: 3000), UUGgugagca (SEQ ID NO: 3001), UUGgugagga (SEQ ID NO: 3002), UUGgugaggg (SEQ ID NO: 3003), UUGgugagua (SEQ ID NO: 3004), UUGgugaguc (SEQ ID NO: 3005), UUGgugagug (SEQ ID NO: 3006), UUGgugaguu (SEQ ID NO: 3007), UUGgugaugg (SEQ ID NO: 3008), UUGgugauua (SEQ ID NO: 3009), UUGgugauug (SEQ ID NO: 3010), UUGgugcaca (SEQ ID NO: 3011), UUGgugggaa (SEQ ID NO: 3012), UUGguggggc (SEQ ID NO: 3013), UUGgugggua (SEQ ID NO: 3014), UUGguggguc (SEQ ID NO: 3015), UUGgugggug (SEQ ID NO: 3016), UUGguggguu (SEQ ID NO: 3017), UUGguguggu (SEQ ID NO: 3018), UUGguguguc (SEQ ID NO: 3019), UUGgugugug (SEQ ID NO: 3020), UUGguguguu (SEQ ID NO: 3021), UUGguuaagu (SEQ ID NO: 3022), UUGguuagca (SEQ ID NO: 3023), UUGguuagug (SEQ ID NO: 3024), UUGguuaguu (SEQ ID NO: 3025), UUGguuggga (SEQ ID NO: 3026), UUGguugguu (SEQ ID NO: 3027), UUGguuugua (SEQ ID NO: 3028), UUGguuuguc (SEQ ID NO: 3029), UUUgcaagug (SEQ ID NO: 3030), UUUguaaaua (SEQ ID NO: 3031), UUUguaaaug (SEQ ID NO: 3032), UUUguaagaa (SEQ ID NO: 3033), UUUguaagac (SEQ ID NO: 3034), UUUguaagag (SEQ ID NO: 3035), UUUguaagca (SEQ ID NO: 3036), UUUguaaggu (SEQ ID NO: 3037), UUUguaagua (SEQ ID NO: 3038), UUUguaaguc (SEQ ID NO: 3039), UUUguaagug (SEQ ID NO: 3040), UUUguaaguu (SEQ ID NO: 3041), UUUguaauuu (SEQ ID NO: 3042), UUUguacagg (SEQ ID NO: 3043), UUUguacgug (SEQ ID NO: 3044), UUUguacuag (SEQ ID NO: 3045), UUUguacugu (SEQ ID NO: 3046), UUUguagguu (SEQ ID NO: 3047), UUUguauccu (SEQ ID NO: 3048), UUUguauguu (SEQ ID NO: 3049), UUUgugagca (SEQ ID NO: 3050), UUUgugagug (SEQ ID NO: 3051), UUUgugcguc (SEQ ID NO: 3052), UUUguguguc (SEQ ID NO: 3053), and uGGguaccug (SEQ ID NO: 3054).

Additional exemplary gene sequences and splice site sequences (e.g., 5′ splice site sequences) include AAGgcaagau (SEQ ID NO: 96), AUGguaugug (SEQ ID NO: 937), GGGgugaggc (SEQ ID NO: 2281), CAGguaggug (SEQ ID NO: 1222), AAGgucagua (SEQ ID NO: 293), AAGguuagag (SEQ ID NO: 3055), AUGgcacuua (SEQ ID NO: 3056), UAAguaaguc (SEQ ID NO: 2423), UGGgugagcu (SEQ ID NO: 3057), CGAgcugggc (SEQ ID NO: 3058), AAAgcacccc (SEQ ID NO: 3059), UAGguggggg (SEQ ID NO: 3060), AGAguaacgu (SEQ ID NO: 3061), UCGgugaugu (SEQ ID NO: 3062), AAUgucaguu (SEQ ID NO: 516), AGGgucugag (SEQ ID NO: 3063), GAGgugacug (SEQ ID NO: 3064), AUGguagguu (SEQ ID NO: 3065), GAGgucuguc (SEQ ID NO: 2000), CAGguaugug (SEQ ID NO: 1260), CAAguacugc (SEQ ID NO: 3066), CACgugcgua (SEQ ID NO: 3067), CCGgugagcu (SEQ ID NO: 3068), CAGguacuuc (SEQ ID NO: 3069), CAGgcgagag (SEQ ID NO: 1115), GAAgcaagua (SEQ ID NO: 3070), AGGgugagca (SEQ ID NO: 789), CAGgcaaguc (SEQ ID NO: 3071), AAGgugaggc (SEQ ID NO: 344), CAGguaagua (SEQ ID NO: 1147), CCAguugggu (SEQ ID NO: 3072), AAGguguggg (SEQ ID NO: 3073), CAGguuggag (SEQ ID NO: 1484), CCGguaugaa (SEQ ID NO: 3074), UGGguaaugu (SEQ ID NO: 2845), CAGgugaggu (SEQ ID NO: 1344), AGAguaauag (SEQ ID NO: 3075), CAGguaugag (SEQ ID NO: 1249), AUGguaaguu (SEQ ID NO: 901), UUGguggguc (SEQ ID NO: 3015), UUUguaagca (SEQ ID NO: 3036), CUCguaugcc (SEQ ID NO: 3076), UAGguaagag (SEQ ID NO: 2483), UAGgcaaguu (SEQ ID NO: 3077), GGAguuaagu (SEQ ID NO: 3078), GAGguaugcc (SEQ ID NO: 1959), AAGguguggu (SEQ ID NO: 402), CAGgugggug (SEQ ID NO: 1415), UUAguaagua (SEQ ID NO: 2933), AAGguuggcu (SEQ ID NO: 3079), UGAguaugug (SEQ ID NO: 3080), CCAgccuucc (SEQ ID NO: 3081), CCUguacgug (SEQ ID NO: 3082), CCUguaggua (SEQ ID NO: 1601), CAGguacgcu (SEQ ID NO: 3083), GAGguucuuc (SEQ ID NO: 3084), AAGguugccu (SEQ ID NO: 3085), CGUguucacu (SEQ ID NO: 3086), CGGgugggga (SEQ ID NO: 3087), UAGgugggau (SEQ ID NO: 2614), CGGguaagga (SEQ ID NO: 3088), AAGguacuau (SEQ ID NO: 195), GGGguaagcu (SEQ ID NO: 2248), ACGguagagc (SEQ ID NO: 3089), CAGgugaaga (SEQ ID NO: 1318), GCGguaagag (SEQ ID NO: 3090), CAGguguugu (SEQ ID NO: 3091), GAAguuugug (SEQ ID NO: 3092), AUGgugagca (SEQ ID NO: 955), CGGguucgug (SEQ ID NO: 3093), AUUguccggc (SEQ ID NO: 3094), GAUgugugug (SEQ ID NO: 3095), AUGgucuguu (SEQ ID NO: 3096), AAGguaggau (SEQ ID NO: 219), CCGguaagau (SEQ ID NO: 1575), AAGguaaaga (SEQ ID NO: 126), GGGgugaguu (SEQ ID NO: 2285), AGGguuggug (SEQ ID NO: 808), GGAgugagug (SEQ ID NO: 2228), AGUguaagga (SEQ ID NO: 3097), UAGguaacug (SEQ ID NO: 2480), AAGgugaaga (SEQ ID NO: 3098), UGGguaagug (SEQ ID NO: 2843), CAGguaagag (SEQ ID NO: 1140), UAGgugagcg (SEQ ID NO: 3099), GAGguaaaaa (SEQ ID NO: 1865), GCCguaaguu (SEQ ID NO: 3100), AAGguuuugu (SEQ ID NO: 473), CAGgugagga (SEQ ID NO: 1341), ACAgcccaug (SEQ ID NO: 3101), GCGgugagcc (SEQ ID NO: 3102), CAGguaugca (SEQ ID NO: 1251), AUGguaccua (SEQ ID NO: 3103), CAAguaugua (SEQ ID NO: 1050), AUGgugguge (SEQ ID NO: 3104), UAAguggcag (SEQ ID NO: 3105), UAGguauagu (SEQ ID NO: 3106), CUGguauuua (SEQ ID NO: 3107), AGGguaaacg (SEQ ID NO: 3108), AUAguaagug (SEQ ID NO: 850), UUGguacuga (SEQ ID NO: 3109), GGUguaagcc (SEQ ID NO: 2303), GAGguggaua (SEQ ID NO: 3110), GAUguaagaa (SEQ ID NO: 3111), ACGgucaguu (SEQ ID NO: 3112), UAAguaaaca (SEQ ID NO: 3113), AAGguaucug (SEQ ID NO: 251), AGGguauuug (SEQ ID NO: 3114), AAGgugaaug (SEQ ID NO: 328), CUGgugaauu (SEQ ID NO: 1749), CAGguuuuuu (SEQ ID NO: 1514), CAUguaugug (SEQ ID NO: 1534), UUGguagagg (SEQ ID NO: 2973), AAGguaugcc (SEQ ID NO: 258), CAGgugccac (SEQ ID NO: 3115), UCGguauuga (SEQ ID NO: 2727), AAGguuugug (SEQ ID NO: 468), AAUguacagg (SEQ ID NO: 3116), CAUguggguu (SEQ ID NO: 1545), CAUgugaguu (SEQ ID NO: 1542), UUGguaaugu (SEQ ID NO: 2968), AGUguaggug (SEQ ID NO: 3117), GAGguaacuc (SEQ ID NO: 3118), GAGguggcgc (SEQ ID NO: 3119), CUGguaauug (SEQ ID NO: 3120), GAGguuugcu (SEQ ID NO: 3121), UGUguacgug (SEQ ID NO: 3122), UAGguaaaga (SEQ ID NO: 2468), CUAguaggca (SEQ ID NO: 3123), UCUgugaguc (SEQ ID NO: 2761), UCUguaaggc (SEQ ID NO: 3124), CAGguuugug (SEQ ID NO: 1509), GAGguagggc (SEQ ID NO: 1935), AAGguaacca (SEQ ID NO: 3125), ACUgugaguu (SEQ ID NO: 646), UAGguaauag (SEQ ID NO: 2495), AAAguaagcu (SEQ ID NO: 17), AUGgugagug (SEQ ID NO: 963), UAGguuugug (SEQ ID NO: 2645), AACguaggac (SEQ ID NO: 3126), GUAgcaggua (SEQ ID NO: 3127), GAGgucagac (SEQ ID NO: 3128), AGGguaugaa (SEQ ID NO: 3129), GAGguuagug (SEQ ID NO: 2089), CAGgcacgug (SEQ ID NO: 3130), GGGgcaagac (SEQ ID NO: 3131), CAGguguguc (SEQ ID NO: 1441), CAGguauuga (SEQ ID NO: 1265), CAGguauguc (SEQ ID NO: 1259), AAGgcaaggu (SEQ ID NO: 3132), UUGgugagaa (SEQ ID NO: 3133), AAGguaaaau (SEQ ID NO: 122), GGGguaagua (SEQ ID NO: 2251), AAGguaucuu (SEQ ID NO: 252), GACgugaguc (SEQ ID NO: 3134), UAUguaugcu (SEQ ID NO: 3135), AAGguacugu (SEQ ID NO: 199), CAGgugaacu (SEQ ID NO: 3136), CACguaaaug (SEQ ID NO: 3137), AAGgugugau (SEQ ID NO: 3138), GAAguauuug (SEQ ID NO: 3139), AAGgucugug (SEQ ID NO: 3140), AAGguggagg (SEQ ID NO: 3141), AAGguauaug (SEQ ID NO: 244), CAGguucuua (SEQ ID NO: 1477), AGGguaacca (SEQ ID NO: 730), CAGgugucac (SEQ ID NO: 1423), AAAguucugu (SEQ ID NO: 3142), UUGgugaguu (SEQ ID NO: 3007), CAAgugaguc (SEQ ID NO: 1067), UAGguagguc (SEQ ID NO: 2525), GCGgugagcu (SEQ ID NO: 2180), AUUgugagga (SEQ ID NO: 3143), CAGgugcaca (SEQ ID NO: 1361), CAGguuggaa (SEQ ID NO: 3144), CUGgucacuu (SEQ ID NO: 3145), GGAguaagug (SEQ ID NO: 2214), GAGgugggcu (SEQ ID NO: 2059), AAGguacuug (SEQ ID NO: 201), AGGguaggau (SEQ ID NO: 3146), AAUguguguu (SEQ ID NO: 3147), ACAguuaagu (SEQ ID NO: 568), GAGgugugug (SEQ ID NO: 2078), AAGgcgggcu (SEQ ID NO: 3148), AUAgcaagua (SEQ ID NO: 3149), AAGguuguua (SEQ ID NO: 454), CAAgcaaggc (SEQ ID NO: 3150), GUGguaauua (SEQ ID NO: 3151), UCUguucagu (SEQ ID NO: 3152), AGGguaggcc (SEQ ID NO: 754), AAGguaucau (SEQ ID NO: 3153), UAGguaccuu (SEQ ID NO: 2509), AAGguaugac (SEQ ID NO: 254), GGAguaggua (SEQ ID NO: 2219), UAAguuggca (SEQ ID NO: 3154), AGUgugagge (SEQ ID NO: 3155), GAGguuugug (SEQ ID NO: 3156), UGGgucugcu (SEQ ID NO: 3157), CAGgugaucc (SEQ ID NO: 1350), CAGgucagug (SEQ ID NO: 1283), AAGguaaggg (SEQ ID NO: 151), CAGgugcagu (SEQ ID NO: 3158), GAGguggguc (SEQ ID NO: 2064), GCUgugagug (SEQ ID NO: 2206), AAGguggagu (SEQ ID NO: 3159), GGGgucaguu (SEQ ID NO: 3160), AGCguaagug (SEQ ID NO: 719), AGAguaugaa (SEQ ID NO: 691), GGGguagggu (SEQ ID NO: 3161), AAGgccagca (SEQ ID NO: 3162), CGAguaugcc (SEQ ID NO: 3163), GUGgugagcg (SEQ ID NO: 3164), AAUguaaauu (SEQ ID NO: 481), CAGgugcgca (SEQ ID NO: 1375), GGUguaugaa (SEQ ID NO: 3165), CUUgugaguu (SEQ ID NO: 1804), AAGguaucuc (SEQ ID NO: 250), AGAguaagga (SEQ ID NO: 665), UAGguaagac (SEQ ID NO: 2482), GAGgugagug (SEQ ID NO: 2026), CAGguguguu (SEQ ID NO: 1443), UUGgugagua (SEQ ID NO: 3004), AGGgcgaguu (SEQ ID NO: 3166), CAGguuuugc (SEQ ID NO: 3167), UUUgugaguu (SEQ ID NO: 3168), AGGguaagca (SEQ ID NO: 736), GAGguccucu (SEQ ID NO: 3169), CCAgcaggua (SEQ ID NO: 3170), GAGguucgcg (SEQ ID NO: 3171), CAGgugaucu (SEQ ID NO: 1351), ACUguaagua (SEQ ID NO: 625), AAGguaaauc (SEQ ID NO: 131), CAGgcaaaua (SEQ ID NO: 3172), GUGguaagca (SEQ ID NO: 2346), CAGguuaaau (SEQ ID NO: 3173), UUGguaauaa (SEQ ID NO: 3174), UAUguaggua (SEQ ID NO: 3175), CAGguaguau (SEQ ID NO: 1225), AAGgugugcc (SEQ ID NO: 3176), UGGguaagag (SEQ ID NO: 2834), CAGgcaagca (SEQ ID NO: 3177), UUGguaaggg (SEQ ID NO: 2961), AAGgcaggug (SEQ ID NO: 109), ACGguaaaug (SEQ ID NO: 3178), GCUgugagca (SEQ ID NO: 3179), AUGguacaca (SEQ ID NO: 3180), GUAguguguu (SEQ ID NO: 3181), ACUguaagag (SEQ ID NO: 3182), CCCgcagguc (SEQ ID NO: 3183), GAGgugagcc (SEQ ID NO: 2019), GAGgugcugu (SEQ ID NO: 3184), UAAguaugcu (SEQ ID NO: 3185), GAGgccaucu (SEQ ID NO: 3186), UCAgugagug (SEQ ID NO: 2700), CAGgugcuac (SEQ ID NO: 3187), AAUgugggug (SEQ ID NO: 533), GAGgugugaa (SEQ ID NO: 3188), CUGguagguc (SEQ ID NO: 1730), GUGgcgcgcg (SEQ ID NO: 3189), CAGgugcaaa (SEQ ID NO: 1359), UAAguggagg (SEQ ID NO: 3190), CAUgugggua (SEQ ID NO: 3191), GAGguagggu (SEQ ID NO: 3192), AAAgugaguu (SEQ ID NO: 61), AGGguucuag (SEQ ID NO: 3193), UGUgugagcu (SEQ ID NO: 3194), AGGgugaauc (SEQ ID NO: 3195), CAGgucaggg (SEQ ID NO: 3196), AAGgucccug (SEQ ID NO: 3197), CUGguagagu (SEQ ID NO: 3198), UAGgucaguu (SEQ ID NO: 2570), AAAguaaggg (SEQ ID NO: 19), CAAguaugug (SEQ ID NO: 1052), CAGgugcuuu (SEQ ID NO: 3199), AAGguaauuc (SEQ ID NO: 169), GGGgugcacg (SEQ ID NO: 3200), ACUgugcuac (SEQ ID NO: 3201), CAGguaccua (SEQ ID NO: 3202), CAGguagcuu (SEQ ID NO: 1211), UGGgugaggc (SEQ ID NO: 2873), CUGguacauu (SEQ ID NO: 1718), AGGguaaucu (SEQ ID NO: 3203), CAGguacaag (SEQ ID NO: 1161), CAGguaauuc (SEQ ID NO: 1157), AGGgcacuug (SEQ ID NO: 3204), UAGgugagaa (SEQ ID NO: 2587), GAGguaaugc (SEQ ID NO: 3205), CCAgugaguu (SEQ ID NO: 3206), AAAguaugug (SEQ ID NO: 44), CUGgugaauc (SEQ ID NO: 3207), UAUguaugua (SEQ ID NO: 2663), CCUgcaggug (SEQ ID NO: 3208), CAGguaucug (SEQ ID NO: 1245), GAGgugaggu (SEQ ID NO: 3209), CUGguaaaac (SEQ ID NO: 3210), UGUgugugcu (SEQ ID NO: 3211), CAGguuaagu (SEQ ID NO: 3212), CAGguaaucc (SEQ ID NO: 1152), UAGguauuug (SEQ ID NO: 3213), UGGguagguc (SEQ ID NO: 2852), CAGguaacag (SEQ ID NO: 1129), AGCgugcgug (SEQ ID NO: 3214), AAGgucagga (SEQ ID NO: 289), GGUgugagcc (SEQ ID NO: 2312), CUGguaagua (SEQ ID NO: 1707), GGGgugggca (SEQ ID NO: 3215), AAGgugggaa (SEQ ID NO: 376), CAGgugagug (SEQ ID NO: 1347), CUGguuguua (SEQ ID NO: 3216), CAGguaauag (SEQ ID NO: 3217), UAGgugaguu (SEQ ID NO: 3218), AGAguaaguu (SEQ ID NO: 671), UAGguaaucc (SEQ ID NO: 3219), CCGgugacug (SEQ ID NO: 3220), GUCgugauua (SEQ ID NO: 3221), CUUguaagug (SEQ ID NO: 1794), UAGguaguca (SEQ ID NO: 3222), CUGguaaguc (SEQ ID NO: 3223), AGGgugagcg (SEQ ID NO: 3224), CAGguaugga (SEQ ID NO: 1255), AUUgugacca (SEQ ID NO: 3225), GUUgugggua (SEQ ID NO: 2411), AAGguacaag (SEQ ID NO: 173), CUAgcaagug (SEQ ID NO: 3226), CUGgugagau (SEQ ID NO: 3227), CAGgugggca (SEQ ID NO: 1406), AUGgcucgag (SEQ ID NO: 3228), CUGguacguu (SEQ ID NO: 1720), UUGgugugua (SEQ ID NO: 3229), GAGgugucug (SEQ ID NO: 3230), GAGgugggac (SEQ ID NO: 3231), GGGgugggag (SEQ ID NO: 3232), GCAgcgugag (SEQ ID NO: 3233), GAGguaaaga (SEQ ID NO: 1870), GAGguaugua (SEQ ID NO: 1965), AAGgugagac (SEQ ID NO: 336), AAGguacaau (SEQ ID NO: 174), CUGguaugag (SEQ ID NO: 3234), AACguaaaau (SEQ ID NO: 3235), GUGguaggga (SEQ ID NO: 2364), CUGguaugug (SEQ ID NO: 1737), CUUguaagca (SEQ ID NO: 3236), AAGguaggga (SEQ ID NO: 223), AUUguaagcc (SEQ ID NO: 3237), AUGguaagcu (SEQ ID NO: 895), CAGgugaauu (SEQ ID NO: 1322), UAGgugaaua (SEQ ID NO: 2581), CAAguaugga (SEQ ID NO: 3238), AUGguauggc (SEQ ID NO: 936), GAGgucaugc (SEQ ID NO: 3239), CAGguacccu (SEQ ID NO: 1174), ACAgugagac (SEQ ID NO: 3240), CAGgucugau (SEQ ID NO: 3241), GAAguugggu (SEQ ID NO: 3242), CUGgugegug (SEQ ID NO: 1767), CAGguacgag (SEQ ID NO: 1180), ACAgugagcc (SEQ ID NO: 556), AAGguaagua (SEQ ID NO: 153), GGAguaaggc (SEQ ID NO: 3243), GAGgugugua (SEQ ID NO: 2077), AAGgucauuu (SEQ ID NO: 3244), CAGguagucu (SEQ ID NO: 3245), AUGguaucug (SEQ ID NO: 3246), AAGguaaacu (SEQ ID NO: 125), GAGguaggug (SEQ ID NO: 1938), CUGguaagca (SEQ ID NO: 1700), AGGguaagag (SEQ ID NO: 734), AAAguaaagc (SEQ ID NO: 3247), CAGguuugag (SEQ ID NO: 1502), GAGgcgggua (SEQ ID NO: 3248), CGAguacgau (SEQ ID NO: 3249), CAGguuguug (SEQ ID NO: 1495), AAAguauggg (SEQ ID NO: 3250), UAGgcugguc (SEQ ID NO: 3251), AAGguaagga (SEQ ID NO: 149), AAGguuuccu (SEQ ID NO: 458), UUGguaaaac (SEQ ID NO: 3252), GAGguaagua (SEQ ID NO: 1893), CAGguucaag (SEQ ID NO: 1465), UGGguuaugu (SEQ ID NO: 3253), GAGgugaguu (SEQ ID NO: 2027), ACGgugaaac (SEQ ID NO: 598), GAUguaacca (SEQ ID NO: 3254), AAGgugcggg (SEQ ID NO: 3255), CCGguacgug (SEQ ID NO: 3256), GAUgugagaa (SEQ ID NO: 3257), GUGgegguga (SEQ ID NO: 3258), CAGguauuag (SEQ ID NO: 3259), GAGguuggga (SEQ ID NO: 3260), AAGgcuagua (SEQ ID NO: 3261), AAGgugggcg (SEQ ID NO: 381), CAGgcaggga (SEQ ID NO: 3262), AAUguuaguu (SEQ ID NO: 3263), GAGguaaagg (SEQ ID NO: 3264), CAGgugugcu (SEQ ID NO: 1437), CUGguaugau (SEQ ID NO: 1733), AUGguuaguc (SEQ ID NO: 978), CUGgugagaa (SEQ ID NO: 1751), CAGgccggcg (SEQ ID NO: 3265), CAGgugacug (SEQ ID NO: 1332), AAAguaaggu (SEQ ID NO: 20), UAAguacuug (SEQ ID NO: 3266), AAGguaaagc (SEQ ID NO: 127), UCGguagggg (SEQ ID NO: 3267), CAGguaggaa (SEQ ID NO: 1212), AGUguaagca (SEQ ID NO: 817), CCCgugagau (SEQ ID NO: 3268), GUGguuguuu (SEQ ID NO: 3269), CAGguuugcc (SEQ ID NO: 1504), AGGguauggg (SEQ ID NO: 766), UAAguaagug (SEQ ID NO: 2424), GAGguaagac (SEQ ID NO: 3270), GAUguagguc (SEQ ID NO: 3271), CAAguaggug (SEQ ID NO: 1043), AUAguaaaua (SEQ ID NO: 845), GAGguugggg (SEQ ID NO: 3272), GAGgcgagua (SEQ ID NO: 3273), CAGguagugu (SEQ ID NO: 1229), GUGguaggug (SEQ ID NO: 2366), CAAgugagug (SEQ ID NO: 1068), AAGgugacaa (SEQ ID NO: 330), CCAgcguaau (SEQ ID NO: 3274), ACGgugaggu (SEQ ID NO: 3275), GGGguauauu (SEQ ID NO: 3276), CAGgugagua (SEQ ID NO: 1345), AAGgugcgug (SEQ ID NO: 364), UAUguaaauu (SEQ ID NO: 3277), CAGgucagua (SEQ ID NO: 1281), ACGguacuua (SEQ ID NO: 3278), GAGgucagca (SEQ ID NO: 3279), UAAguaugua (SEQ ID NO: 2431), GGGgucagac (SEQ ID NO: 3280), AAUgugugag (SEQ ID NO: 3281), UCCgucagua (SEQ ID NO: 3282), CAGgugcuuc (SEQ ID NO: 1391), CCAguuagug (SEQ ID NO: 3283), CCGgugggcg (SEQ ID NO: 1590), AGGgugcaug (SEQ ID NO: 3284), GGGguaggau (SEQ ID NO: 3285), UAGgugggcc (SEQ ID NO: 2615), GAGguguucg (SEQ ID NO: 3286), UUGgcaagaa (SEQ ID NO: 3287), UCCguaagua (SEQ ID NO: 3288), CAGguguaag (SEQ ID NO: 3289), CUCgugagua (SEQ ID NO: 1680), GAGguguuuu (SEQ ID NO: 3290), GAGgugagca (SEQ ID NO: 2018), GAGguaaagu (SEQ ID NO: 1872), AAGguacguu (SEQ ID NO: 193), CAGguccagu (SEQ ID NO: 1291), AUGgugaaac (SEQ ID NO: 947), GUAgugagcu (SEQ ID NO: 3291), CAGgugaaaa (SEQ ID NO: 3292), AGGguacagg (SEQ ID NO: 3293), AAGguaacgc (SEQ ID NO: 3294), AAGguauacc (SEQ ID NO: 3295), CCUgugagau (SEQ ID NO: 3296), GGGguacgug (SEQ ID NO: 3297), GAGguauggu (SEQ ID NO: 1964), UAGguauuau (SEQ ID NO: 2557), GAAguaggag (SEQ ID NO: 3298), UCGguaaggg (SEQ ID NO: 3299), CCGguaagcg (SEQ ID NO: 3300), GAAguaauua (SEQ ID NO: 1823), CAGgugaguc (SEQ ID NO: 1346), AAGgucaaga (SEQ ID NO: 279), AUGguaaguc (SEQ ID NO: 899), CAGgugagcu (SEQ ID NO: 1340), CCAguuuuug (SEQ ID NO: 3301), CAGgugggag (SEQ ID NO: 1404), AAGguauuau (SEQ ID NO: 270), AAGguaaaua (SEQ ID NO: 130), AAGgugcugu (SEQ ID NO: 3302), AAAguacacc (SEQ ID NO: 3303), CUGguucgug (SEQ ID NO: 1783), UCAguaaguc (SEQ ID NO: 2690), GAAguacgug (SEQ ID NO: 3304), CAGgugacaa (SEQ ID NO: 1323), UGGguaagaa (SEQ ID NO: 2832), UGUguagggg (SEQ ID NO: 3305), GAGguaggca (SEQ ID NO: 1932), UUGgugaggc (SEQ ID NO: 3306), AUGgugugua (SEQ ID NO: 974), CAGguccucc (SEQ ID NO: 3307), UUGguaaaug (SEQ ID NO: 2953), GCUgugaguu (SEQ ID NO: 2207), AUGgucugua (SEQ ID NO: 3308), CAUgcaggug (SEQ ID NO: 3309), CUGguacacc (SEQ ID NO: 3310), CAGguccuua (SEQ ID NO: 3311), CAAguaaucu (SEQ ID NO: 1031), AUGgcagccu (SEQ ID NO: 3312), AAGgucagaa (SEQ ID NO: 282), AACgugaggc (SEQ ID NO: 3313), CAGgcacgca (SEQ ID NO: 1106), ACGguccagg (SEQ ID NO: 3314), UCUguacaua (SEQ ID NO: 3315), GAGgugauua (SEQ ID NO: 3316), ACGguaaaua (SEQ ID NO: 3317), AUGguaacug (SEQ ID NO: 3318), CAGgcgcguu (SEQ ID NO: 3319), CAGguauaga (SEQ ID NO: 1235), AAGguuuguu (SEQ ID NO: 3320), CAGguaugaa (SEQ ID NO: 1247), UAGguuggua (SEQ ID NO: 2638), CUGgugagac (SEQ ID NO: 1752), CAGguuagga (SEQ ID NO: 3321), AUGgugacug (SEQ ID NO: 3322), UUGguauccc (SEQ ID NO: 3323), CUUguaggac (SEQ ID NO: 3324), AAAguguguu (SEQ ID NO: 69), CAGguuucuu (SEQ ID NO: 1500), GGGguauggc (SEQ ID NO: 3325), GGGguaggac (SEQ ID NO: 3326), ACUguaaguc (SEQ ID NO: 626), AUCguaagcu (SEQ ID NO: 3327), UAGguucccc (SEQ ID NO: 2636), GGUgugagca (SEQ ID NO: 3328), CUGguuggua (SEQ ID NO: 3329), GGGguuaggg (SEQ ID NO: 3330), UGAguaagaa (SEQ ID NO: 3331), GAGguauucc (SEQ ID NO: 1969), UGGguuaguc (SEQ ID NO: 2893), CAGgcucgug (SEQ ID NO: 3332), UAGguagagu (SEQ ID NO: 3333), UAGgugcccu (SEQ ID NO: 3334), AAAgugagua (SEQ ID NO: 58), GAGguucaua (SEQ ID NO: 2094), UUGguaagag (SEQ ID NO: 2958), ACCgugugua (SEQ ID NO: 3335), UAUguaguau (SEQ ID NO: 3336), UGGguaauag (SEQ ID NO: 3337), CAGgucugaa (SEQ ID NO: 3338), AAAguauaaa (SEQ ID NO: 3339), GUGgugaguc (SEQ ID NO: 3340), AGUgugauua (SEQ ID NO: 3341), UUGgugugug (SEQ ID NO: 3020), CAGgugaugg (SEQ ID NO: 1353), GCUgugagua (SEQ ID NO: 2204), CAGguacaug (SEQ ID NO: 1169), AAGguacagu (SEQ ID NO: 178), GAAguuguag (SEQ ID NO: 3342), CAGgugauua (SEQ ID NO: 1355), UAGgugaauu (SEQ ID NO: 2583), GGUguuaaua (SEQ ID NO: 3343), CAGguauuua (SEQ ID NO: 1268), CAAguacucg (SEQ ID NO: 3344), CAAguaagaa (SEQ ID NO: 1022), AAGguaccuu (SEQ ID NO: 188), ACGgugaggg (SEQ ID NO: 3345), UGAgcaggca (SEQ ID NO: 3346), GGGgugaccg (SEQ ID NO: 3347), GAGguaaaug (SEQ ID NO: 1875), CGGguuugug (SEQ ID NO: 3348), AAGgugagcg (SEQ ID NO: 341), GUGguaugga (SEQ ID NO: 3349), CUGguaagga (SEQ ID NO: 1703), GAGguaccag (SEQ ID NO: 1911), CCGgugagug (SEQ ID NO: 1587), AAGguuagaa (SEQ ID NO: 416), GAGguacuug (SEQ ID NO: 1921), AGAguaaaac (SEQ ID NO: 651), UCUgugagua (SEQ ID NO: 2760), AAGgcgggaa (SEQ ID NO: 3350), CAGguaugcg (SEQ ID NO: 1253), AGGguaaaac (SEQ ID NO: 3351), AAGgugacug (SEQ ID NO: 333), AGGguauguu (SEQ ID NO: 3352), AAGguaugua (SEQ ID NO: 263), CAGgucucuc (SEQ ID NO: 1302), CAGgcaugua (SEQ ID NO: 3353), CUGguaggua (SEQ ID NO: 1729), AAGgucaugc (SEQ ID NO: 3354), CAGguacaca (SEQ ID NO: 1163), GAUguacguu (SEQ ID NO: 3355), ACAguacgug (SEQ ID NO: 3356), ACGguaccca (SEQ ID NO: 3357), CAGguagugc (SEQ ID NO: 3358), ACAguaagag (SEQ ID NO: 3359), GGUgcacacc (SEQ ID NO: 3360), GAGguguaac (SEQ ID NO: 3361), AAGgugugua (SEQ ID NO: 403), UAGguacuua (SEQ ID NO: 3362), GCGguacugc (SEQ ID NO: 3363), UGGguaaguc (SEQ ID NO: 2842), CAUguaggua (SEQ ID NO: 1529), CAGguaggau (SEQ ID NO: 3364), CAGgucuggc (SEQ ID NO: 3365), GUGguuuuaa (SEQ ID NO: 3366), CAGgugggaa (SEQ ID NO: 1402), UGGgugagua (SEQ ID NO: 2875), CGAgugagcc (SEQ ID NO: 3367), AAGguauggc (SEQ ID NO: 261), AGUguuguca (SEQ ID NO: 3368), CAGgugauuu (SEQ ID NO: 1358), UAGguaucuc (SEQ ID NO: 2544), UAAguauguu (SEQ ID NO: 3369), AAGguugagc (SEQ ID NO: 3370), AGAguaaaga (SEQ ID NO: 653), GGUguaagua (SEQ ID NO: 3371), GGGgugagcu (SEQ ID NO: 2279), CAGguauaau (SEQ ID NO: 3372), GAGguacaaa (SEQ ID NO: 1904), AUGguaccaa (SEQ ID NO: 3373), UAGguagggg (SEQ ID NO: 2523), UGAgucagaa (SEQ ID NO: 3374), AAGgcaauua (SEQ ID NO: 3375), UUGguaagau (SEQ ID NO: 3376), CAGguacaga (SEQ ID NO: 1165), AGAguuagag (SEQ ID NO: 3377), CAGgugcguc (SEQ ID NO: 1381), GAGguauuac (SEQ ID NO: 3378), ACGguacaga (SEQ ID NO: 3379), CAGgucuucc (SEQ ID NO: 1313), AAGguaaggu (SEQ ID NO: 152), GAGguaauuu (SEQ ID NO: 1903), AGUguaggcu (SEQ ID NO: 3380), AAAguaagcg (SEQ ID NO: 3381), CCUguaagcc (SEQ ID NO: 3382), AGGgugauuu (SEQ ID NO: 3383), UGUguaugaa (SEQ ID NO: 3384), CUGguacaca (SEQ ID NO: 3385), AGGguagaga (SEQ ID NO: 3386), AUAguaagca (SEQ ID NO: 848), AGAguaugua (SEQ ID NO: 3387), UUGgucagca (SEQ ID NO: 3388), CAGgcaaguu (SEQ ID NO: 1105), AAGguauaua (SEQ ID NO: 242), AAGgucugga (SEQ ID NO: 314), CAGguacgca (SEQ ID NO: 1181), AGGgugcggg (SEQ ID NO: 3389), AUGguaagug (SEQ ID NO: 900), AAAgugauga (SEQ ID NO: 3390), UGCgugagua (SEQ ID NO: 3391), AGAguaggga (SEQ ID NO: 684), UGUguaggua (SEQ ID NO: 2912), UAGguaggau (SEQ ID NO: 2521), UAAgugagug (SEQ ID NO: 2440), GCUguaagua (SEQ ID NO: 2193), GAAguaagaa (SEQ ID NO: 1814), UCGgugaggc (SEQ ID NO: 2733), UAGguauuuu (SEQ ID NO: 2564), AAGguacaca (SEQ ID NO: 3392), AAGguaggua (SEQ ID NO: 227), UGGguagguu (SEQ ID NO: 2854), ACAgcaagua (SEQ ID NO: 541), GAGguaggag (SEQ ID NO: 1931), UGGgugaguu (SEQ ID NO: 2878), GCGgugagau (SEQ ID NO: 3393), CCUguagguu (SEQ ID NO: 3394), CAGgugugua (SEQ ID NO: 1440), CUGguaagcc (SEQ ID NO: 1701), AAGgugauuc (SEQ ID NO: 3395), CAGguagcua (SEQ ID NO: 1208), GUUguaagug (SEQ ID NO: 3396), AUGguaagca (SEQ ID NO: 893), AUAguaggga (SEQ ID NO: 3397), GGGguucgcu (SEQ ID NO: 3398), CCGgucagag (SEQ ID NO: 3399), GUAguaugag (SEQ ID NO: 3400), CGUguaagau (SEQ ID NO: 3401), UGAguaggca (SEQ ID NO: 3402), UCAguaugua (SEQ ID NO: 3403), GAGguaucug (SEQ ID NO: 1954), AGAguauuuu (SEQ ID NO: 3404), AAGguuguag (SEQ ID NO: 3405), AGUguaaguu (SEQ ID NO: 821), CGGguaaguu (SEQ ID NO: 1626), UCGgugcgga (SEQ ID NO: 3406), UAGguaagua (SEQ ID NO: 2491), GAAguuagau (SEQ ID NO: 3407), GCUgugagac (SEQ ID NO: 3408), CAGgcaggua (SEQ ID NO: 3409), CAGguagggg (SEQ ID NO: 1218), UAAguuaaga (SEQ ID NO: 3410), AUGguggguu (SEQ ID NO: 970), UAGguaaguu (SEQ ID NO: 2494), CUGguaaauu (SEQ ID NO: 1690), CCGguaagga (SEQ ID NO: 1577), GAGgcaggca (SEQ ID NO: 3411), CAUguaagug (SEQ ID NO: 1523), AAGgugccua (SEQ ID NO: 3412), UUGguaggga (SEQ ID NO: 2977), AAGguaaaca (SEQ ID NO: 123), CGGgugugag (SEQ ID NO: 3413), GGGgugugag (SEQ ID NO: 3414), UCCguggguc (SEQ ID NO: 3415), ACGguaaauc (SEQ ID NO: 3416), UCAguaggua (SEQ ID NO: 3417), CAGgucagcc (SEQ ID NO: 1278), CAGgcggugg (SEQ ID NO: 3418), CGAguaagcu (SEQ ID NO: 3419), CCCgugagca (SEQ ID NO: 3420), AAAguaauga (SEQ ID NO: 3421), CUGguaagcu (SEQ ID NO: 1702), CGGguaacca (SEQ ID NO: 3422), CAGgucgcac (SEQ ID NO: 3423), GAGguaggcc (SEQ ID NO: 3424), UAGgugagcc (SEQ ID NO: 2591), UAGguaggca (SEQ ID NO: 3425), GCGgugcgug (SEQ ID NO: 3426), AUGgugagua (SEQ ID NO: 961), GGGgugaggg (SEQ ID NO: 2282), GAGgucacac (SEQ ID NO: 3427), CAGguaggcc (SEQ ID NO: 3428), CAAgugcuga (SEQ ID NO: 3429), GUCgucuuca (SEQ ID NO: 3430), CAUguaagaa (SEQ ID NO: 1518), GUAguaagga (SEQ ID NO: 3431), UAGguuugua (SEQ ID NO: 2643), CAAguuagag (SEQ ID NO: 3432), AAGguagagu (SEQ ID NO: 208), AAGgugagau (SEQ ID NO: 338), AAAguaggua (SEQ ID NO: 37), ACAgugaauc (SEQ ID NO: 3433), CAGgugugcg (SEQ ID NO: 1436), CAGgucggcc (SEQ ID NO: 1299), AAGguaguau (SEQ ID NO: 3434), ACUgucaguc (SEQ ID NO: 3435), UCUgcagccu (SEQ ID NO: 3436), CGAguaagug (SEQ ID NO: 3437), AGAguaauua (SEQ ID NO: 3438), AGUgugagug (SEQ ID NO: 837), CCGgugagcg (SEQ ID NO: 3439), AAGguaaccu (SEQ ID NO: 3440), AAGguugugg (SEQ ID NO: 3441), AAGgcauggg (SEQ ID NO: 3442), AAGgucagag (SEQ ID NO: 284), ACGguaaggu (SEQ ID NO: 3443), GGGgugagca (SEQ ID NO: 3444), GAGguugcuu (SEQ ID NO: 3445), AAGguaucgc (SEQ ID NO: 3446), CCGguaaagg (SEQ ID NO: 3447), AAAguuaaug (SEQ ID NO: 3448), UAGguacgag (SEQ ID NO: 2510), ACCguaauua (SEQ ID NO: 3449), GGGguaagga (SEQ ID NO: 2249), CCGguaacgc (SEQ ID NO: 3450), CAGgucagaa (SEQ ID NO: 1275), AAGguacuga (SEQ ID NO: 197), GAGgugacca (SEQ ID NO: 2010), GGGgugagcc (SEQ ID NO: 2277), AAGguacagg (SEQ ID NO: 177), AUGguaauua (SEQ ID NO: 3451), CAGgugagag (SEQ ID NO: 1335), AAGgugacuc (SEQ ID NO: 3452), AUAguaagua (SEQ ID NO: 849), GAGguaaacc (SEQ ID NO: 1869), CAGgugggau (SEQ ID NO: 1405), CAGgugagaa (SEQ ID NO: 1333), AGGguaaaaa (SEQ ID NO: 3453), GAGgugugac (SEQ ID NO: 3454), CACguaagcu (SEQ ID NO: 3455), CAGguccccc (SEQ ID NO: 3456), CAGgucaggu (SEQ ID NO: 3457), CGGguaaguc (SEQ ID NO: 3458), ACGguauggg (SEQ ID NO: 3459), GAUguaaguu (SEQ ID NO: 2123), CAAguaauau (SEQ ID NO: 3460), CAGguugggg (SEQ ID NO: 3461), CCUgugcugg (SEQ ID NO: 3462), AAGguaugau (SEQ ID NO: 256), AGGguagagg (SEQ ID NO: 3463), AAGguggguu (SEQ ID NO: 386), CAGgugugaa (SEQ ID NO: 1430), UUGguaugug (SEQ ID NO: 2988), UUGguaucuc (SEQ ID NO: 2985), GGGgugagug (SEQ ID NO: 2284), CUGgugugug (SEQ ID NO: 1779), AGGguagggc (SEQ ID NO: 3464), GUGgugagua (SEQ ID NO: 3465), CAGguaugua (SEQ ID NO: 1258), AAGguacauu (SEQ ID NO: 181), UUAguaagug (SEQ ID NO: 2934), AAUguauauc (SEQ ID NO: 3466), CUUguaagua (SEQ ID NO: 1793), GAGguuagua (SEQ ID NO: 2087), CAGguaaggu (SEQ ID NO: 1146), CAGguaaugu (SEQ ID NO: 1155), AGGgugaggc (SEQ ID NO: 3467), CAGguauuuc (SEQ ID NO: 1269), CAGgucugga (SEQ ID NO: 1307), GGGgugugcu (SEQ ID NO: 3468), UAGgugagug (SEQ ID NO: 2598), AAUguaaccu (SEQ ID NO: 3469), UAAgugaguc (SEQ ID NO: 2439), CAGgugcacu (SEQ ID NO: 3470), ACGguaagua (SEQ ID NO: 579), GAGguauccu (SEQ ID NO: 3471), UCUguaaguc (SEQ ID NO: 2745), CAGguauuca (SEQ ID NO: 1263), UGUguaagug (SEQ ID NO: 3472), CCAgcaaggc (SEQ ID NO: 3473), GAGgugaagg (SEQ ID NO: 2006), AAUguggggu (SEQ ID NO: 3474), UCGgugcgug (SEQ ID NO: 3475), UUGguaaggc (SEQ ID NO: 3476), GAGguaagug (SEQ ID NO: 3477), AAAguaagau (SEQ ID NO: 14), UAGgucuuuu (SEQ ID NO: 3478), GAGgucugau (SEQ ID NO: 3479), CCAguuagag (SEQ ID NO: 3480), UGGgugaaaa (SEQ ID NO: 3481), AGAguaagau (SEQ ID NO: 662), CAGguaauug (SEQ ID NO: 1158), CAGgccgguc (SEQ ID NO: 3482), CCGguaagag (SEQ ID NO: 3483), GAGgugagcu (SEQ ID NO: 2021), CUGguaagac (SEQ ID NO: 3484), CAGgugagau (SEQ ID NO: 1336), CUGguuuguu (SEQ ID NO: 3485), UGGguaggua (SEQ ID NO: 3486), CAGguuagug (SEQ ID NO: 1457), CAGguguucg (SEQ ID NO: 3487), CGGguagguc (SEQ ID NO: 3488), GUGguacaua (SEQ ID NO: 3489), AAGguacuaa (SEQ ID NO: 194), GAUgugagua (SEQ ID NO: 3490), UGUguaagac (SEQ ID NO: 2904), GAGguagccg (SEQ ID NO: 3491), UAGgugaucu (SEQ ID NO: 3492), CAGguacgug (SEQ ID NO: 1185), CUUgucaguc (SEQ ID NO: 3493), GAGguaucac (SEQ ID NO: 3494), GAGguaauga (SEQ ID NO: 3495), AAGguaacac (SEQ ID NO: 3496), CAGguaaagc (SEQ ID NO: 1123), AAGgcaagua (SEQ ID NO: 3497), CGCgugagcc (SEQ ID NO: 3498), AGUgugcguu (SEQ ID NO: 3499), GAUguaagca (SEQ ID NO: 2118), AAGguaauag (SEQ ID NO: 159), GGAgcaguug (SEQ ID NO: 3500), AGCguaagau (SEQ ID NO: 3501), AAGgucaggc (SEQ ID NO: 290), GAGguauuca (SEQ ID NO: 3502), AAUguaaagu (SEQ ID NO: 3503), CAGguaacaa (SEQ ID NO: 3504), UCGguaggug (SEQ ID NO: 3505), AAAguaaguc (SEQ ID NO: 22), CGGgugcagu (SEQ ID NO: 3506), GGUgugugca (SEQ ID NO: 3507), UGAgugagaa (SEQ ID NO: 2794), CACguguaag (SEQ ID NO: 3508), GUGguuggua (SEQ ID NO: 3509), GCAgccuuga (SEQ ID NO: 3510), CGAgugugau (SEQ ID NO: 3511), CAGguauaua (SEQ ID NO: 3512), UAUguaugug (SEQ ID NO: 2665), CCCgugguca (SEQ ID NO: 3513), AUGguaagac (SEQ ID NO: 890), GAGgugugga (SEQ ID NO: 2074), AGUguauccu (SEQ ID NO: 3514), UGAguguguc (SEQ ID NO: 3515), UGGguaaucu (SEQ ID NO: 3516), AUGgcagguu (SEQ ID NO: 3517), GAGguaagau (SEQ ID NO: 1884), UCAgcagcgu (SEQ ID NO: 3518), AAGgugggau (SEQ ID NO: 378), CGGgugcgcu (SEQ ID NO: 3519), CAGgugucug (SEQ ID NO: 1429), AGCgugguaa (SEQ ID NO: 3520), AAUgugaaug (SEQ ID NO: 3521), UCGgugagac (SEQ ID NO: 3522), UAGguaaagc (SEQ ID NO: 3523), CUGguaaaag (SEQ ID NO: 3524), CCGgugcgga (SEQ ID NO: 3525), CAGguacuca (SEQ ID NO: 3526), CAGguagcaa (SEQ ID NO: 1203), GAAguugagu (SEQ ID NO: 3527), GAGguggagg (SEQ ID NO: 2052), AGGguaugag (SEQ ID NO: 762), UAGguaugcu (SEQ ID NO: 3528), UAGgugagac (SEQ ID NO: 2588), CAGguaauua (SEQ ID NO: 1156), CGUguaagcc (SEQ ID NO: 3529), CUUguaaguu (SEQ ID NO: 1795), AAGguaacuu (SEQ ID NO: 140), UCGgcaaggc (SEQ ID NO: 3530), GAGguucucg (SEQ ID NO: 3531), GAGgugggcg (SEQ ID NO: 2058), AAGgcaugug (SEQ ID NO: 3532), CUGguauguu (SEQ ID NO: 1738), UAAgucauuu (SEQ ID NO: 3533), CAUguaauua (SEQ ID NO: 1525), AAUguaaaga (SEQ ID NO: 3534), UAGgugcuca (SEQ ID NO: 3535), AAGguaaugg (SEQ ID NO: 166), GAGguacuga (SEQ ID NO: 3536), UGGguaagua (SEQ ID NO: 2841), UGGguaaaaa (SEQ ID NO: 3537), AAGgugagcu (SEQ ID NO: 342), UACgugaguu (SEQ ID NO: 3538), AGGgugagcc (SEQ ID NO: 790), CGGgugagga (SEQ ID NO: 3539), UGGgugagag (SEQ ID NO: 2869), GGUguaagcu (SEQ ID NO: 3540), CGGguggguu (SEQ ID NO: 1648), CCAgcuaagu (SEQ ID NO: 3541), AAGguuuguc (SEQ ID NO: 467), GAGguuagac (SEQ ID NO: 2084), GAGguaccuc (SEQ ID NO: 3542), UUUguaaguu (SEQ ID NO: 3041), GAGguuagga (SEQ ID NO: 3543), CAGguaggga (SEQ ID NO: 1216), AGGguaauac (SEQ ID NO: 744), UGCgugugua (SEQ ID NO: 3544), CCAguaacca (SEQ ID NO: 3545), AGGgucuguc (SEQ ID NO: 3546), UGGguaugua (SEQ ID NO: 2860), GUGguaagcu (SEQ ID NO: 2348), CAGguaaccu (SEQ ID NO: 3547), AAGgugaguu (SEQ ID NO: 350), UAGguucgug (SEQ ID NO: 3548), AAAguuagua (SEQ ID NO: 3549), UGGgcaaguc (SEQ ID NO: 2816), AAGgcacagu (SEQ ID NO: 3550), GUUguaaguc (SEQ ID NO: 2401), AAGguuugcc (SEQ ID NO: 462), CUUgcauggg (SEQ ID NO: 3551), GCGgugagua (SEQ ID NO: 3552), GGGguaagcg (SEQ ID NO: 3553), GCCguaagaa (SEQ ID NO: 3554), GAGgucggga (SEQ ID NO: 3555), UUGguauugu (SEQ ID NO: 2990), AGUgugagac (SEQ ID NO: 3556), CUGgugggga (SEQ ID NO: 1770), AGAguaaggu (SEQ ID NO: 668), CCGguggguc (SEQ ID NO: 3557), CAGguauucu (SEQ ID NO: 1264), UGGguaacgu (SEQ ID NO: 3558), UUGgugagag (SEQ ID NO: 3559), UAGguacccu (SEQ ID NO: 3560), GGGgugcguc (SEQ ID NO: 3561), AAGgcaggag (SEQ ID NO: 3562), ACGguacauu (SEQ ID NO: 3563), GAGguaguua (SEQ ID NO: 1946), CAGguauggg (SEQ ID NO: 1256), UUUguguguc (SEQ ID NO: 3053), CAGguacuua (SEQ ID NO: 1194), AUGguauacu (SEQ ID NO: 3564), AGUgugagcc (SEQ ID NO: 833), ACAguaacga (SEQ ID NO: 3565), CUGguaccca (SEQ ID NO: 3566), CAGguaaccc (SEQ ID NO: 3567), GGAguaagua (SEQ ID NO: 3568), GAGgugggug (SEQ ID NO: 2065), ACUguauguc (SEQ ID NO: 3569), ACGgugagua (SEQ ID NO: 606), CUGguaaugu (SEQ ID NO: 3570), AAGguaucag (SEQ ID NO: 247), CAGgugcccc (SEQ ID NO: 1370), AGUgucagug (SEQ ID NO: 3571), AAGguaggag (SEQ ID NO: 218), GGAguaugug (SEQ ID NO: 3572), UUGguauuuu (SEQ ID NO: 2992), CCUguuguga (SEQ ID NO: 3573), UUUguaagaa (SEQ ID NO: 3033), UAGguaacau (SEQ ID NO: 2475), CAGguaagca (SEQ ID NO: 3574), CAGgucacag (SEQ ID NO: 3575), CAGgugugag (SEQ ID NO: 1432), UAGguuugcg (SEQ ID NO: 3576), CUGguaagaa (SEQ ID NO: 1697), ACGguuguau (SEQ ID NO: 3577), AAGguugggg (SEQ ID NO: 446), AAGgugaauu (SEQ ID NO: 329), GGGguuaguu (SEQ ID NO: 3578), ACGguaaggc (SEQ ID NO: 3579), CAGguuuaag (SEQ ID NO: 1496), CUGguaaguu (SEQ ID NO: 1709), GGGgugagag (SEQ ID NO: 3580), UGGguggguu (SEQ ID NO: 2886), GAGguuuguu (SEQ ID NO: 2111), UGGguaaaug (SEQ ID NO: 2826), CAGgcaggcc (SEQ ID NO: 3581), CACgugcagg (SEQ ID NO: 3582), AAGgugagcc (SEQ ID NO: 340), CAAguaagug (SEQ ID NO: 1028), CAGgucaguc (SEQ ID NO: 1282), GCGguauaau (SEQ ID NO: 3583), UAGguaaagu (SEQ ID NO: 3584), UAGguggauu (SEQ ID NO: 3585), GAGgucugga (SEQ ID NO: 3586), UCGgucaguu (SEQ ID NO: 3587), UGGguaacug (SEQ ID NO: 3588), AAGguuugau (SEQ ID NO: 3589), UGUgcuggug (SEQ ID NO: 3590), UGUguaccuc (SEQ ID NO: 3591), UGGguacagu (SEQ ID NO: 2849), AUCgucagcg (SEQ ID NO: 3592), CAGgucuugg (SEQ ID NO: 3593), GAAguuggua (SEQ ID NO: 3594), GAAguaaaga (SEQ ID NO: 3595), UUGguaagcu (SEQ ID NO: 2959), UAGguaccag (SEQ ID NO: 2507), AGGguaucau (SEQ ID NO: 3596), CAGguaaaaa (SEQ ID NO: 1118), ACGguaauuu (SEQ ID NO: 583), AUUguaaguu (SEQ ID NO: 997), GAGguacagu (SEQ ID NO: 1908), CAGgugaaag (SEQ ID NO: 1315), UGGguuguuu (SEQ ID NO: 3597), GGGguaggug (SEQ ID NO: 2259), CAGgugccca (SEQ ID NO: 1369), AGCgugagau (SEQ ID NO: 3598), CCAgugagug (SEQ ID NO: 1565), AGGguagaug (SEQ ID NO: 3599), UGGguguguc (SEQ ID NO: 2888), AUCgcgugag (SEQ ID NO: 3600), AGGguaagcc (SEQ ID NO: 3601), AGGguagcag (SEQ ID NO: 3602), UUCguuuccg (SEQ ID NO: 3603), AAGguaagcg (SEQ ID NO: 147), UGGguaagcc (SEQ ID NO: 2837), CAGguauggc (SEQ ID NO: 3604), UGUguaagua (SEQ ID NO: 2907), AAGguagaga (SEQ ID NO: 3605), ACGguaauaa (SEQ ID NO: 3606), CUGguacggu (SEQ ID NO: 3607), GAGgucacag (SEQ ID NO: 3608), UAUguaaguu (SEQ ID NO: 2656), CUGguacgcc (SEQ ID NO: 3609), CAAguaagau (SEQ ID NO: 1024), CUAgugagua (SEQ ID NO: 1673), CCGguaaccg (SEQ ID NO: 3610), CUUguaaguc (SEQ ID NO: 3611), GUGgugagaa (SEQ ID NO: 2378), ACCguaugua (SEQ ID NO: 3612), GUAguaagug (SEQ ID NO: 2324), UUGgugggua (SEQ ID NO: 3014), CGGguacuuu (SEQ ID NO: 3613), UGGguaaaua (SEQ ID NO: 2825), AGAgugagua (SEQ ID NO: 704), AAGguagguu (SEQ ID NO: 230), AAGguaugcg (SEQ ID NO: 3614), CCUguaggcu (SEQ ID NO: 3615), ACAguagaaa (SEQ ID NO: 3616), CCGguuagua (SEQ ID NO: 3617), CGGguaggcg (SEQ ID NO: 3618), GCAgugagug (SEQ ID NO: 2162), GAGgugaguc (SEQ ID NO: 3619), CUGguagccu (SEQ ID NO: 3620), CAUguaugua (SEQ ID NO: 1533), GAAguaacuu (SEQ ID NO: 3621), GAAguaagau (SEQ ID NO: 3622), AAGguuagau (SEQ ID NO: 417), AAGguaauca (SEQ ID NO: 161), AAUguaugua (SEQ ID NO: 507), UGAguaagau (SEQ ID NO: 2767), AGAgugagca (SEQ ID NO: 703), GUAguucuau (SEQ ID NO: 3623), GAGguaauca (SEQ ID NO: 1898), UAGguaugga (SEQ ID NO: 2548), UAGgugggac (SEQ ID NO: 2612), GAGguacaug (SEQ ID NO: 3624), UGGguaaggc (SEQ ID NO: 3625), CAGguacgcc (SEQ ID NO: 1182), CCAguuacgc (SEQ ID NO: 3626), ACUgugguga (SEQ ID NO: 3627), GAGguaaguc (SEQ ID NO: 1894), AUUguaggug (SEQ ID NO: 3628), ACCgucagug (SEQ ID NO: 3629), AAUgugaggg (SEQ ID NO: 3630), ACUgugagug (SEQ ID NO: 645), UGGguguggu (SEQ ID NO: 3631), AAGguuggga (SEQ ID NO: 445), AAGguuugga (SEQ ID NO: 464), UCCgugagug (SEQ ID NO: 3632), CGGgugagug (SEQ ID NO: 1642), AGAguaagcu (SEQ ID NO: 664), CAGgcaagcu (SEQ ID NO: 3633), UAGguauauu (SEQ ID NO: 2541), AAAguagcag (SEQ ID NO: 3634), GAGguaaccu (SEQ ID NO: 1880), AAGgugggca (SEQ ID NO: 379), AGGgugagua (SEQ ID NO: 795), UGGguaaggu (SEQ ID NO: 2840), CUUgucagug (SEQ ID NO: 3635), UAGgugcgcu (SEQ ID NO: 3636), GAGgcaaauu (SEQ ID NO: 3637), AGGguaccuc (SEQ ID NO: 3638), CAAgugcgua (SEQ ID NO: 3639), AGAguaagac (SEQ ID NO: 660), GUGguaaaua (SEQ ID NO: 3640), GAUguaagcg (SEQ ID NO: 3641), GAGguaaagc (SEQ ID NO: 1871), UAGgugagua (SEQ ID NO: 2596), CAGguaacau (SEQ ID NO: 1130), CCUguacggc (SEQ ID NO: 3642), UAGguauguc (SEQ ID NO: 2552), UAGguccaua (SEQ ID NO: 3643), GAGgugaaaa (SEQ ID NO: 2003), AAAguacuga (SEQ ID NO: 3644), UUGguaagcg (SEQ ID NO: 3645), CAGgcaagcg (SEQ ID NO: 3646), UUUgcagguu (SEQ ID NO: 3647), CAGguuuaua (SEQ ID NO: 3648), CUGguaaagc (SEQ ID NO: 1686), AUGgugagcu (SEQ ID NO: 958), CAGgugguug (SEQ ID NO: 1419), GUAguaaguu (SEQ ID NO: 3649), CAGguaauac (SEQ ID NO: 3650), CAGgcaaggc (SEQ ID NO: 3651), AAGguaauuu (SEQ ID NO: 171), UUUguccgug (SEQ ID NO: 3652), GAGguagguu (SEQ ID NO: 1939), ACCgugagug (SEQ ID NO: 3653), CAAguaagcu (SEQ ID NO: 3654), ACAgugagua (SEQ ID NO: 560), UUGgugagau (SEQ ID NO: 3000), AAGguagucu (SEQ ID NO: 233), CAGguaaagg (SEQ ID NO: 3655), GGGguaugga (SEQ ID NO: 2264), UUUguaagug (SEQ ID NO: 3040), GUGguaagag (SEQ ID NO: 2344), AGUgugaguu (SEQ ID NO: 838), AAGgcaagcg (SEQ ID NO: 3656), UAAgugagua (SEQ ID NO: 2438), AGGgugagug (SEQ ID NO: 797), AGUguacgug (SEQ ID NO: 3657), AGGgugcgua (SEQ ID NO: 3658), GGCgugagcc (SEQ ID NO: 2238), CGAguuauga (SEQ ID NO: 3659), CAGguaaaga (SEQ ID NO: 1122), UUGgugaaga (SEQ ID NO: 3660), AGGguaaugg (SEQ ID NO: 3661), AAGguccaga (SEQ ID NO: 300), AGUgugaguc (SEQ ID NO: 836), CAGguaauuu (SEQ ID NO: 1159), CAGguaacgc (SEQ ID NO: 3662), CUGguacacu (SEQ ID NO: 3663), CUGguuagug (SEQ ID NO: 1782), CAGguacuug (SEQ ID NO: 3664), CACguaagua (SEQ ID NO: 3665), GUGgugcggc (SEQ ID NO: 3666), GAGgucaguu (SEQ ID NO: 3667), AUGguaugcc (SEQ ID NO: 932), AAGgugugug (SEQ ID NO: 405), CUGguggguc (SEQ ID NO: 1772), CAGgugaggc (SEQ ID NO: 1342), AAGguuaguc (SEQ ID NO: 423), AAGguagcug (SEQ ID NO: 215), GAGgucagga (SEQ ID NO: 1983), GUUguaggua (SEQ ID NO: 3668), UGGguacaag (SEQ ID NO: 3669), AUGguaggug (SEQ ID NO: 924), GAGguaagcc (SEQ ID NO: 1886), AUGgcaagua (SEQ ID NO: 3670), AAGguauauu (SEQ ID NO: 245), GCGgugagag (SEQ ID NO: 3671), AAGgugcuuc (SEQ ID NO: 3672), UAGguacauc (SEQ ID NO: 3673), ACUgugguaa (SEQ ID NO: 3674), GAGguaggcu (SEQ ID NO: 1933), GAGguaugca (SEQ ID NO: 3675), AGGguaguuc (SEQ ID NO: 3676), CAGguauccu (SEQ ID NO: 1241), AGGguaaguc (SEQ ID NO: 741), AGGgucaguu (SEQ ID NO: 779), CAGguuggga (SEQ ID NO: 3677), CAGguggaua (SEQ ID NO: 3678), GGAguagguu (SEQ ID NO: 2220), GAGguaggau (SEQ ID NO: 3679), GGGguuugug (SEQ ID NO: 3680), UAGguaauug (SEQ ID NO: 3681), AAGguaaccc (SEQ ID NO: 136), ACGguaagaa (SEQ ID NO: 3682), GAGguagggg (SEQ ID NO: 1936), CGAguaggug (SEQ ID NO: 1619), UCCguaagug (SEQ ID NO: 2710), UCGguacagg (SEQ ID NO: 3683), CAAguaagcg (SEQ ID NO: 3684), AAGguccgcg (SEQ ID NO: 3685), AAUgugagua (SEQ ID NO: 523), CAGgugaaug (SEQ ID NO: 3686), GUGguaaggc (SEQ ID NO: 2350), AGAgugagug (SEQ ID NO: 706), UCUguauguc (SEQ ID NO: 3687), UGGgugaguc (SEQ ID NO: 2876), UCGguuagua (SEQ ID NO: 3688), GAUguaugca (SEQ ID NO: 3689), GAGguuggug (SEQ ID NO: 3690), GAGguggggc (SEQ ID NO: 2061), UGGgucaguc (SEQ ID NO: 3691), GCAgugagua (SEQ ID NO: 2161), CAGguugcuu (SEQ ID NO: 3692), AGGguagagu (SEQ ID NO: 3693), UAGgucaggu (SEQ ID NO: 2567), CGCguaugua (SEQ ID NO: 3694), GAGguauuaa (SEQ ID NO: 3695), CAGguaaacu (SEQ ID NO: 3696), AAAguaaguu (SEQ ID NO: 24), GGGgucuggc (SEQ ID NO: 3697), GCUguggggu (SEQ ID NO: 3698), UUGguaaguc (SEQ ID NO: 3699), AAGguagaag (SEQ ID NO: 3700), AAUgugaguc (SEQ ID NO: 524), AAGgucagcu (SEQ ID NO: 288), AAGguaagag (SEQ ID NO: 143), AUGgugagga (SEQ ID NO: 3701), AAGguacuuc (SEQ ID NO: 200), AAGguaagaa (SEQ ID NO: 141), CCGguacagc (SEQ ID NO: 3702), GCGgugcgga (SEQ ID NO: 3703), CAGguacaua (SEQ ID NO: 1168), CUGgugagga (SEQ ID NO: 1755), CUGguaggug (SEQ ID NO: 1731), AACguagguu (SEQ ID NO: 3704), AUGgugugug (SEQ ID NO: 975), UUGguacuau (SEQ ID NO: 3705), CAGgucggug (SEQ ID NO: 1300), CAGgcauggg (SEQ ID NO: 3706), AUGguaucuu (SEQ ID NO: 929), AAGguaacua (SEQ ID NO: 137), CAGgugggcg (SEQ ID NO: 3707), CACgugagga (SEQ ID NO: 3708), AAGgugguuc (SEQ ID NO: 392), UGGgcauucu (SEQ ID NO: 3709), AUGguaagcc (SEQ ID NO: 894), AGGgucagug (SEQ ID NO: 778), AGAguacgua (SEQ ID NO: 3710), AAGguaggca (SEQ ID NO: 220), AAGguauuca (SEQ ID NO: 3711), CAGguagauu (SEQ ID NO: 1202), GAGguauuua (SEQ ID NO: 1972), GAGgucuaca (SEQ ID NO: 3712), GUUguagguc (SEQ ID NO: 3713), CAGguacucg (SEQ ID NO: 3714), GUCguauguu (SEQ ID NO: 3715), AAGguacuuu (SEQ ID NO: 202), AGAgugagau (SEQ ID NO: 702), AGUguuggua (SEQ ID NO: 3716), AAUgugagug (SEQ ID NO: 525), AAGguagauu (SEQ ID NO: 3717), AUGguuugua (SEQ ID NO: 988), GAGgccccag (SEQ ID NO: 3718), AUGgucaguu (SEQ ID NO: 3719), UCUguaagga (SEQ ID NO: 3720), CAGgucgggc (SEQ ID NO: 3721), CAGguaagcc (SEQ ID NO: 1142), UAGgucagug (SEQ ID NO: 2569), AGAguaggaa (SEQ ID NO: 683), CUGguacuuc (SEQ ID NO: 3722), CUCguaagca (SEQ ID NO: 1674), CAGguaacua (SEQ ID NO: 1134), CAGguggcug (SEQ ID NO: 1401), UGGguccgua (SEQ ID NO: 3723), GAGguugugc (SEQ ID NO: 3724), CAGgugcgcg (SEQ ID NO: 1377), AAAguauggc (SEQ ID NO: 3725), UGAguacgua (SEQ ID NO: 2779), CUGguacgga (SEQ ID NO: 3726), CAAgugaccu (SEQ ID NO: 3727), AAGgugaugu (SEQ ID NO: 356), AAGgucugca (SEQ ID NO: 3728), AAAguuugua (SEQ ID NO: 75), AAGgugagca (SEQ ID NO: 339), GAUguaagcc (SEQ ID NO: 2119), CAAguaauuu (SEQ ID NO: 1035), CAGgugugug (SEQ ID NO: 1442), UGGgugaggg (SEQ ID NO: 2874), AAGgugaccu (SEQ ID NO: 3729), UAGgugugag (SEQ ID NO: 2621), CAGgcagguc (SEQ ID NO: 3730), UCAguaaguu (SEQ ID NO: 2692), UCAgcaguga (SEQ ID NO: 3731), AAGguaccac (SEQ ID NO: 3732), UAAguaggug (SEQ ID NO: 3733), AAGgucagcc (SEQ ID NO: 286), CAGguaacuc (SEQ ID NO: 1135), AAAguaagag (SEQ ID NO: 13), AAGguagaua (SEQ ID NO: 209), AAGgcaaggg (SEQ ID NO: 99), CAGgugucgg (SEQ ID NO: 3734), CAGguggcua (SEQ ID NO: 3735), GAGguugcca (SEQ ID NO: 3736), CAGgccgugg (SEQ ID NO: 3737), UUGguauaug (SEQ ID NO: 3738), GAGguugagu (SEQ ID NO: 3739), GAGguagguc (SEQ ID NO: 3740), GUGguaagac (SEQ ID NO: 2343), UAGguccuuc (SEQ ID NO: 3741), GAGgcaaguc (SEQ ID NO: 3742), GAGguaacau (SEQ ID NO: 3743), CAGguauauc (SEQ ID NO: 1236), UCGguugguu (SEQ ID NO: 3744), CAGgugaacc (SEQ ID NO: 3745), CAGgucuuuu (SEQ ID NO: 3746), CAGgcauggc (SEQ ID NO: 3747), AAAguacuug (SEQ ID NO: 32), CAGgugauuc (SEQ ID NO: 1356), UUGguagguu (SEQ ID NO: 3748), UAUgugagca (SEQ ID NO: 3749), CAGgugagcg (SEQ ID NO: 1339), AAUguaauaa (SEQ ID NO: 3750), AAAguaaggc (SEQ ID NO: 3751), UAGguuuguc (SEQ ID NO: 2644), UAGgugggag (SEQ ID NO: 2613), GAGguaaguu (SEQ ID NO: 3752), AAGguagccg (SEQ ID NO: 3753), CAGguggugc (SEQ ID NO: 3754), UGAgucaguu (SEQ ID NO: 3755), CUGguaggcc (SEQ ID NO: 3756), CAAguaagga (SEQ ID NO: 3757), CGGguaaggc (SEQ ID NO: 3758), AAGgcgagga (SEQ ID NO: 3759), CAGguaguuc (SEQ ID NO: 1230), CAGguaagga (SEQ ID NO: 1143), CCUgugagug (SEQ ID NO: 1610), AAGguaaaug (SEQ ID NO: 132), CCGguaauua (SEQ ID NO: 3760), CAGguaaguu (SEQ ID NO: 1149), AAGgugguca (SEQ ID NO: 3761), CAGguaccuc (SEQ ID NO: 1177), AUCguaagua (SEQ ID NO: 3762), CCGguacaua (SEQ ID NO: 3763), GCGgugagug (SEQ ID NO: 3764), GAGgugguau (SEQ ID NO: 2067), CUGgugugga (SEQ ID NO: 3765), GAGguaauuc (SEQ ID NO: 3766), CAAguacgua (SEQ ID NO: 3767), UCUguaagug (SEQ ID NO: 2746), AAUguaagug (SEQ ID NO: 491), AGGgucuguu (SEQ ID NO: 783), GAGguacugc (SEQ ID NO: 1918), AGGguaaggc (SEQ ID NO: 738), AAGgcaagag (SEQ ID NO: 95), CAGguggguu (SEQ ID NO: 1416), UAGguuagga (SEQ ID NO: 3768), UGAguaagcu (SEQ ID NO: 2769), AGAguaagag (SEQ ID NO: 661), AUGgcaggug (SEQ ID NO: 3769), UAGgcaagua (SEQ ID NO: 3770), AUGguaggua (SEQ ID NO: 923), GCAgcccgca (SEQ ID NO: 3771), ACGguaaacu (SEQ ID NO: 3772), AGGgugaguu (SEQ ID NO: 798), GUAguagucu (SEQ ID NO: 3773), GUGgcugaaa (SEQ ID NO: 3774), CAGguuaguc (SEQ ID NO: 1456), CUGgugagca (SEQ ID NO: 1753), UCAguaagug (SEQ ID NO: 2691), AAAgugauug (SEQ ID NO: 3775), UAGgucugga (SEQ ID NO: 3776), GAGguguuuc (SEQ ID NO: 3777), AAGguaaauu (SEQ ID NO: 133), CAUguacauc (SEQ ID NO: 3778), AAGguuugaa (SEQ ID NO: 3779), CCAgcaagug (SEQ ID NO: 3780), UAGguaauaa (SEQ ID NO: 3781), GAGgcaagug (SEQ ID NO: 1859), CAAgugauuc (SEQ ID NO: 1071), CAGgucgugg (SEQ ID NO: 3782), GAAguaugcc (SEQ ID NO: 3783), UCGgugcccu (SEQ ID NO: 3784), GAGgucaguc (SEQ ID NO: 3785), CAGgugagac (SEQ ID NO: 1334), UUUgucugua (SEQ ID NO: 3786), CAGguagaua (SEQ ID NO: 3787), UGGguaucag (SEQ ID NO: 3788), UAGgugggcu (SEQ ID NO: 2616), AUGgugagau (SEQ ID NO: 3789), CAGguaacac (SEQ ID NO: 3790), CCGguauccu (SEQ ID NO: 3791), UAGguaagcu (SEQ ID NO: 2487), UCAguacauc (SEQ ID NO: 3792), UAGguuugcc (SEQ ID NO: 2642), AUGguaagaa (SEQ ID NO: 889), UUGguaagac (SEQ ID NO: 3793), CCGguuaguc (SEQ ID NO: 3794), GAGguaagaa (SEQ ID NO: 1882), UGGguaaguu (SEQ ID NO: 2844), CCGgugagaa (SEQ ID NO: 1585), CCUgugaggg (SEQ ID NO: 1608), ACGguaggag (SEQ ID NO: 590), ACAguauguc (SEQ ID NO: 3795), CAGguauuaa (SEQ ID NO: 3796), CAGguggauc (SEQ ID NO: 3797), AGAgugcgua (SEQ ID NO: 3798), AAGgugaccg (SEQ ID NO: 3799), AGAguaggug (SEQ ID NO: 687), ACUguaugua (SEQ ID NO: 3800), UAGgucaauu (SEQ ID NO: 3801), AGUguguaag (SEQ ID NO: 3802), CGGguaccuu (SEQ ID NO: 3803), CUAgugaguu (SEQ ID NO: 3804), CUAguaagug (SEQ ID NO: 1666), CAGguacaac (SEQ ID NO: 3805), UAGgugugug (SEQ ID NO: 2627), CAUguacggc (SEQ ID NO: 3806), AUGgugugag (SEQ ID NO: 3807), AGGguggaag (SEQ ID NO: 3808), CAGgugcgag (SEQ ID NO: 3809), UAGgugcucc (SEQ ID NO: 3810), AAGguggugg (SEQ ID NO: 390), AAGgucuguu (SEQ ID NO: 317), CAGgugggcc (SEQ ID NO: 1407), AAGgucaguc (SEQ ID NO: 294), CAGguuuuua (SEQ ID NO: 3811), AACgugaggu (SEQ ID NO: 3812), CGGguaagag (SEQ ID NO: 3813), UUUgucggua (SEQ ID NO: 3814), UAGguuaagu (SEQ ID NO: 3815), GUGguaagaa (SEQ ID NO: 2342), CAGguauugg (SEQ ID NO: 1266), GCUguaaguu (SEQ ID NO: 2196), CUAguaagua (SEQ ID NO: 1664), UCGguaaaua (SEQ ID NO: 3816), CAGguaacuu (SEQ ID NO: 1137), CCUgugagua (SEQ ID NO: 3817), CAGguuauau (SEQ ID NO: 3818), CUGgugaaca (SEQ ID NO: 3819), AAGguauaaa (SEQ ID NO: 238), GAGguaagca (SEQ ID NO: 1885), AAGgugaagc (SEQ ID NO: 324), CAGgugaguu (SEQ ID NO: 1348), UUUgugagua (SEQ ID NO: 3820), CUUguacgcc (SEQ ID NO: 3821), AGAguaagug (SEQ ID NO: 670), UGGguaggug (SEQ ID NO: 2853), UGAgcccuge (SEQ ID NO: 3822), UGUguaugua (SEQ ID NO: 3823), AAGguagagg (SEQ ID NO: 3824), GAGguggggg (SEQ ID NO: 2062), UAGguaauuc (SEQ ID NO: 2502), AAGgcauggu (SEQ ID NO: 3825), AGAguaagca (SEQ ID NO: 663), AAGguaggaa (SEQ ID NO: 217), CAAguaagua (SEQ ID NO: 1026), ACUguaauug (SEQ ID NO: 3826), CAGgucugug (SEQ ID NO: 1311), UCGguaccga (SEQ ID NO: 3827), CUGgugagag (SEQ ID NO: 3828), AAGguuugcu (SEQ ID NO: 463), AUGguaccac (SEQ ID NO: 3829), UAAguuaguu (SEQ ID NO: 3830), CAGguaggac (SEQ ID NO: 1213), AGAgugaggc (SEQ ID NO: 3831), CGAgucagua (SEQ ID NO: 3832), CAGgucugag (SEQ ID NO: 1304), GAGguggugg (SEQ ID NO: 3833), ACGguauugg (SEQ ID NO: 3834), GCUgcgagua (SEQ ID NO: 3835), CUGguaagug (SEQ ID NO: 1708), GUGgugagau (SEQ ID NO: 2379), GGGguuugau (SEQ ID NO: 3836), UCUgugagug (SEQ ID NO: 2762), CUUgucagua (SEQ ID NO: 1801), GAGguaaaac (SEQ ID NO: 1866), UCUguaagau (SEQ ID NO: 2741), CCAguaaguu (SEQ ID NO: 1558), CAGguaaagu (SEQ ID NO: 1124), GCGgugagca (SEQ ID NO: 2179), UAAguaagag (SEQ ID NO: 2416), CUGgcaggug (SEQ ID NO: 3837), GAGguaaggg (SEQ ID NO: 1891), UGAguaaguu (SEQ ID NO: 2775), GAGgugagac (SEQ ID NO: 2015), GCUgucuguu (SEQ ID NO: 3838), AAGguaacaa (SEQ ID NO: 134), GAGguaacgg (SEQ ID NO: 3839), CUGguauucu (SEQ ID NO: 3840), CAAguaacug (SEQ ID NO: 1021), AAGguggggu (SEQ ID NO: 383), UAGguauggc (SEQ ID NO: 2549), CAGguauuuu (SEQ ID NO: 1271), GUGguaaacu (SEQ ID NO: 3841), GAGgucugag (SEQ ID NO: 1998), CUGguaaggu (SEQ ID NO: 1706), CAAguaaguu (SEQ ID NO: 1029), AAGguagacc (SEQ ID NO: 206), GAGgcgagcg (SEQ ID NO: 3842), CUGguaaaua (SEQ ID NO: 1687), UGUguaagcg (SEQ ID NO: 3843), CAGguuaggg (SEQ ID NO: 1453), GGGgugagga (SEQ ID NO: 2280), ACAguaugug (SEQ ID NO: 3844), CCGgugggga (SEQ ID NO: 3845), GAGgucagug (SEQ ID NO: 3846), AGGguaaggu (SEQ ID NO: 3847), ACAguaagua (SEQ ID NO: 546), GGUguaaggu (SEQ ID NO: 3848), GAGguaauaa (SEQ ID NO: 1895), CAGguauucc (SEQ ID NO: 3849), CUGguauaaa (SEQ ID NO: 3850), CCGgucugug (SEQ ID NO: 3851), CAGguaacug (SEQ ID NO: 1136), GCAguaagua (SEQ ID NO: 2147), AAGguagggg (SEQ ID NO: 225), CAAguccacc (SEQ ID NO: 3852), CAAguuggug (SEQ ID NO: 3853), CAGgugcggu (SEQ ID NO: 1379), CAGguaaaau (SEQ ID NO: 3854), ACGguaagga (SEQ ID NO: 3855), UGGguaauaa (SEQ ID NO: 3856), UAGguaagug (SEQ ID NO: 2493), CCGguagguu (SEQ ID NO: 3857), AGAguaugga (SEQ ID NO: 3858), CUCgugaguc (SEQ ID NO: 3859), AAAgccggug (SEQ ID NO: 3860), UUGguaauuu (SEQ ID NO: 2970), GAGguaaaag (SEQ ID NO: 1867), CCUgugugag (SEQ ID NO: 3861), AAAguaagga (SEQ ID NO: 18), UGAgugagug (SEQ ID NO: 2800), AAGguacaug (SEQ ID NO: 180), CCGguaaaug (SEQ ID NO: 3862), CAGgugaagc (SEQ ID NO: 3863), CAGguacccg (SEQ ID NO: 1173), GAGguaaggc (SEQ ID NO: 1890), UUUguauguu (SEQ ID NO: 3049), CAGgugcucc (SEQ ID NO: 1386), UCGguagguc (SEQ ID NO: 3864), CGGgugaggc (SEQ ID NO: 3865), AAGguaauua (SEQ ID NO: 168), ACUgugaguc (SEQ ID NO: 644), AAGgucagca (SEQ ID NO: 285), GUGgugagug (SEQ ID NO: 2384), CAUguccacc (SEQ ID NO: 3866), AAGgugaccc (SEQ ID NO: 3867), CGGguuagua (SEQ ID NO: 3868), GCGguaguaa (SEQ ID NO: 3869), GCUguaggua (SEQ ID NO: 3870), CCUguugagu (SEQ ID NO: 3871), UAGgucuggc (SEQ ID NO: 2577), GAUgugagcc (SEQ ID NO: 2131), CUUgugagua (SEQ ID NO: 1802), CUGguguguu (SEQ ID NO: 1780), GAGgcaugug (SEQ ID NO: 1863), CAGgcaagag (SEQ ID NO: 1101), UUGguaagaa (SEQ ID NO: 2957), GAGguguggg (SEQ ID NO: 2075), GAGguauuuu (SEQ ID NO: 1975), CAGguaguaa (SEQ ID NO: 1224), AGGguaagac (SEQ ID NO: 3872), UUUguaggca (SEQ ID NO: 3873), AGGgugagau (SEQ ID NO: 3874), GAGguuugua (SEQ ID NO: 2110), AAGgugagug (SEQ ID NO: 349), GAGgugggag (SEQ ID NO: 2055), AAGgugagaa (SEQ ID NO: 335), CUGguaagag (SEQ ID NO: 1698), AUAguaaaga (SEQ ID NO: 3875), GAUgugaguc (SEQ ID NO: 2134), AAGgugcagg (SEQ ID NO: 3876), CAGgucuguc (SEQ ID NO: 1310), GAGgugauuu (SEQ ID NO: 3877), CAGguuggcu (SEQ ID NO: 3878), CGGguauggg (SEQ ID NO: 3879), AUGguccauc (SEQ ID NO: 3880), CCGguuggug (SEQ ID NO: 3881), GGAguaaguc (SEQ ID NO: 3882), AAUguaagga (SEQ ID NO: 488), CAGguuuguu (SEQ ID NO: 1510), UAGgugugua (SEQ ID NO: 2626), UAUgucuuug (SEQ ID NO: 3883), ACGguacuuc (SEQ ID NO: 3884), AAGgcacgcg (SEQ ID NO: 3885), CUGguaaacc (SEQ ID NO: 1684), CUUgugggua (SEQ ID NO: 3886), UGAguaaguc (SEQ ID NO: 2773), CUGgugggug (SEQ ID NO: 1773), GAGguggaga (SEQ ID NO: 3887), GUGguggcug (SEQ ID NO: 3888), GUGguaagug (SEQ ID NO: 2353), AACgugagua (SEQ ID NO: 3889), GAAgcuguaa (SEQ ID NO: 3890), CGGguaucuu (SEQ ID NO: 3891), CAGgugucag (SEQ ID NO: 1424), AAUguacgca (SEQ ID NO: 3892), CCGgugggua (SEQ ID NO: 3893), UGGgugaggu (SEQ ID NO: 3894), AAGguauguu (SEQ ID NO: 266), CAGguauguu (SEQ ID NO: 1261), CAGguuugcu (SEQ ID NO: 1505), UUGguaaguu (SEQ ID NO: 2964), CAGguaguug (SEQ ID NO: 1231), CCUgugaaua (SEQ ID NO: 3895), GCUgugugug (SEQ ID NO: 3896), CAAguaauuc (SEQ ID NO: 1033), AGGguaaugu (SEQ ID NO: 3897), GCUgugaguc (SEQ ID NO: 2205), ACCguaaguu (SEQ ID NO: 3898), CGUguaagua (SEQ ID NO: 3899), GGGguaaguc (SEQ ID NO: 3900), AAUguaugau (SEQ ID NO: 3901), AAUgugauua (SEQ ID NO: 3902), UCAguaagaa (SEQ ID NO: 2682), CAGguccguc (SEQ ID NO: 3903), GAAguauuga (SEQ ID NO: 3904), UUGguaagga (SEQ ID NO: 2960), CAGgucgguu (SEQ ID NO: 3905), UAGguuagug (SEQ ID NO: 2635), ACGguaaaac (SEQ ID NO: 577), AAGguagguc (SEQ ID NO: 228), UACgugagua (SEQ ID NO: 3906), UUGguaagca (SEQ ID NO: 3907), GCGgugaguc (SEQ ID NO: 3908), GAAguaaggg (SEQ ID NO: 3909), CGCgugaguu (SEQ ID NO: 3910), CAGguacccc (SEQ ID NO: 3911), UCUguaagac (SEQ ID NO: 3912), GAGgugggca (SEQ ID NO: 2057), AAUguaagac (SEQ ID NO: 3913), CAGgcaaggg (SEQ ID NO: 3914), CAAguaacua (SEQ ID NO: 1020), AAAguuuguc (SEQ ID NO: 3915), CAGguacugu (SEQ ID NO: 1193), AAGgucccuc (SEQ ID NO: 303), UCGguaaguc (SEQ ID NO: 3916), UGGgugagug (SEQ ID NO: 2877), CUUgugagau (SEQ ID NO: 3917), AGAgugagcu (SEQ ID NO: 3918), UAAgugggga (SEQ ID NO: 3919), UAGguaggga (SEQ ID NO: 2522), CAGguuagcc (SEQ ID NO: 1452), AGGguaauca (SEQ ID NO: 3920), AAGguucagc (SEQ ID NO: 3921), UGGgugggug (SEQ ID NO: 2885), CAGguuguga (SEQ ID NO: 1494), AAGguaagug (SEQ ID NO: 155), CAUgugcgua (SEQ ID NO: 1543), CCGguauauu (SEQ ID NO: 3922), ACCguaugug (SEQ ID NO: 3923), CAGguauagu (SEQ ID NO: 3924), CAGguauuac (SEQ ID NO: 3925), CAGgugcagg (SEQ ID NO: 1364), GUGgugagcu (SEQ ID NO: 2381), AAGguaacau (SEQ ID NO: 135), CUGgugaugg (SEQ ID NO: 3926), AUGguaaaug (SEQ ID NO: 882), CCGgugagca (SEQ ID NO: 3927), AAGguaaacc (SEQ ID NO: 124), AAGguacugg (SEQ ID NO: 3928), GCGgucagga (SEQ ID NO: 3929), CUGgucaggg (SEQ ID NO: 3930), AAAguacguu (SEQ ID NO: 3931), AGAguagguu (SEQ ID NO: 688), AGGguaagcu (SEQ ID NO: 3932), AUUgugagua (SEQ ID NO: 1009), CCGgccacca (SEQ ID NO: 3933), GAGguaacuu (SEQ ID NO: 1881), GAGguaugaa (SEQ ID NO: 1956), CAGgucagac (SEQ ID NO: 1276), UAGgcgugug (SEQ ID NO: 2462), AGGguaaguu (SEQ ID NO: 743), CAGgcaugag (SEQ ID NO: 1111), CAGguaacgu (SEQ ID NO: 1133), CAGgcgagca (SEQ ID NO: 3934), UAGguauggu (SEQ ID NO: 2550), AGAguaggau (SEQ ID NO: 3935), CUGguuucaa (SEQ ID NO: 3936), GAGguaaacu (SEQ ID NO: 3937), CAGgcaugca (SEQ ID NO: 1112), UUGguaaucu (SEQ ID NO: 3938), AGGgcagaau (SEQ ID NO: 3939), AUGguaaaac (SEQ ID NO: 877), GCUgcaggug (SEQ ID NO: 3940), GAAgcacgug (SEQ ID NO: 3941), CAUguaaaca (SEQ ID NO: 3942), UGGguaagau (SEQ ID NO: 2835), AGGguagcua (SEQ ID NO: 3943), AGGguggggu (SEQ ID NO: 800), CCUguaaguu (SEQ ID NO: 1600), UGAgugaguu (SEQ ID NO: 2801), GGAguaugua (SEQ ID NO: 3944), CAGgugaccu (SEQ ID NO: 1328), AAAguacgga (SEQ ID NO: 3945), GAGguacaga (SEQ ID NO: 1906), GAUguaggua (SEQ ID NO: 2125), GGGguaauug (SEQ ID NO: 3946), UAGguggguu (SEQ ID NO: 2617), GUGguacgua (SEQ ID NO: 3947), AAGguacagc (SEQ ID NO: 3948), GAGgugaaga (SEQ ID NO: 3949), GGGguaagca (SEQ ID NO: 2246), UGAguagguc (SEQ ID NO: 3950), GGGguaaguu (SEQ ID NO: 2253), AUUgugaguu (SEQ ID NO: 1011), UCAguaagac (SEQ ID NO: 3951), AGUgugagcu (SEQ ID NO: 834), AAGgcaaaac (SEQ ID NO: 3952), CUGgugaguc (SEQ ID NO: 1760), AAGgucucug (SEQ ID NO: 310), GAGgcugugc (SEQ ID NO: 3953), AGAgugagac (SEQ ID NO: 700), GAGgugaugu (SEQ ID NO: 2033), AGAguauggu (SEQ ID NO: 3954), UGGguggguc (SEQ ID NO: 2884), GCUgcugagc (SEQ ID NO: 3955), CAGguagcug (SEQ ID NO: 1210), UAGgucagaa (SEQ ID NO: 3956), CCGguaggug (SEQ ID NO: 3957), GCAguaugau (SEQ ID NO: 3958), CAGguuucag (SEQ ID NO: 3959), GAGguuugcc (SEQ ID NO: 3960), GGGguggggg (SEQ ID NO: 3961), AAGguacaua (SEQ ID NO: 179), UGGguguguu (SEQ ID NO: 2890), AGAguaaggc (SEQ ID NO: 666), GCGguuagug (SEQ ID NO: 3962), AAGgugacuu (SEQ ID NO: 334), AUGguaagau (SEQ ID NO: 892), AUGguaguug (SEQ ID NO: 3963), CAUguaagac (SEQ ID NO: 3964), CUGguaugua (SEQ ID NO: 1736), UUCguaagga (SEQ ID NO: 3965), GAAguaugac (SEQ ID NO: 3966), CGGguaauuc (SEQ ID NO: 1627), UGGguaacuu (SEQ ID NO: 2831), CAGgugccua (SEQ ID NO: 1372), CAUguagggc (SEQ ID NO: 3967), ACCgucagga (SEQ ID NO: 3968), CGUguucgau (SEQ ID NO: 3969), GAGgcaggac (SEQ ID NO: 3970), UAGguaauau (SEQ ID NO: 2496), UCGguauacu (SEQ ID NO: 3971), UAGguugugc (SEQ ID NO: 3972), CCGgugaguc (SEQ ID NO: 3973), CAGgugccaa (SEQ ID NO: 1368), CAGgugaugc (SEQ ID NO: 1352), AAGgugagga (SEQ ID NO: 343), GUGgugaggg (SEQ ID NO: 3974), UGGgucagua (SEQ ID NO: 3975), GAGgucaggg (SEQ ID NO: 1985), UAGguacgua (SEQ ID NO: 2511), GAGgcaagag (SEQ ID NO: 1857), CCUguuggua (SEQ ID NO: 3976), GAGguaucca (SEQ ID NO: 3977), UAAguaagcu (SEQ ID NO: 2419), AAGgucaguu (SEQ ID NO: 296), AAAguuaaag (SEQ ID NO: 3978), GAGgugcuau (SEQ ID NO: 3979), ACGguaaguu (SEQ ID NO: 581), CUGgugaggg (SEQ ID NO: 1757), GAGguuaugu (SEQ ID NO: 2091), CUUgugugca (SEQ ID NO: 3980), UGAgcugggg (SEQ ID NO: 3981), AAGguauagu (SEQ ID NO: 3982), UAGguaaaac (SEQ ID NO: 2464), GGGgugaggu (SEQ ID NO: 3983), GAGgcaagca (SEQ ID NO: 3984), GGAguaacgu (SEQ ID NO: 3985), AGAguaagua (SEQ ID NO: 3986), AAAguaagua (SEQ ID NO: 21), GAGgcaacca (SEQ ID NO: 3987), UGUguaaguu (SEQ ID NO: 2909), UAGgugaggc (SEQ ID NO: 2594), ACAguaagaa (SEQ ID NO: 544), UGAguaagug (SEQ ID NO: 2774), CAAgucagua (SEQ ID NO: 1057), AGGguaaaug (SEQ ID NO: 3988), AAGguaugca (SEQ ID NO: 257), GCUgugcgug (SEQ ID NO: 3989), GAGguucgcc (SEQ ID NO: 3990), AAGgcuugca (SEQ ID NO: 3991), CAGgcaagug (SEQ ID NO: 1104), AUAguaaguc (SEQ ID NO: 3992), UUGguaggua (SEQ ID NO: 2978), GCAgcaggua (SEQ ID NO: 3993), AAGguauauc (SEQ ID NO: 243), AGCguaagcc (SEQ ID NO: 3994), CUGguucgaa (SEQ ID NO: 3995), ACGgugggug (SEQ ID NO: 612), CUGgucauug (SEQ ID NO: 3996), CAGgucagga (SEQ ID NO: 1280), CAAgugagac (SEQ ID NO: 1062), GAGguacugg (SEQ ID NO: 1919), GAGguguagu (SEQ ID NO: 3997), GAGguguccu (SEQ ID NO: 3998), CAGgugcgua (SEQ ID NO: 1380), AGUgcccuga (SEQ ID NO: 3999), AUGgugaguc (SEQ ID NO: 962), UGUgugugua (SEQ ID NO: 4000), CAGguaugcu (SEQ ID NO: 1254), CUGguacagu (SEQ ID NO: 4001), UUGguacgua (SEQ ID NO: 4002), UCUguacgua (SEQ ID NO: 4003), UAAguaauuc (SEQ ID NO: 4004), CACguaugug (SEQ ID NO: 4005), CAGgcaagua (SEQ ID NO: 1103), UCGgugagug (SEQ ID NO: 4006), GGUgugaguc (SEQ ID NO: 2315), UCUguaagcu (SEQ ID NO: 2743), AAGguucaga (SEQ ID NO: 4007), AGGguacuuc (SEQ ID NO: 4008), GCGgcagguu (SEQ ID NO: 4009), GAGgcccgug (SEQ ID NO: 4010), CAGguauaaa (SEQ ID NO: 4011), AUGgucaagu (SEQ ID NO: 4012), AAGgugagua (SEQ ID NO: 347), GUGguuuguu (SEQ ID NO: 4013), AGAgugagga (SEQ ID NO: 4014), GAGguaugac (SEQ ID NO: 1957), UAGgcgugag (SEQ ID NO: 4015), AAGguacucc (SEQ ID NO: 4016), UGAgugagga (SEQ ID NO: 2798), GAGguaugau (SEQ ID NO: 4017), GGGgucggua (SEQ ID NO: 4018), ACGguaugca (SEQ ID NO: 4019), CAGguaccac (SEQ ID NO: 1171), UAAguaccug (SEQ ID NO: 4020), AGGgugggcu (SEQ ID NO: 4021), CUGgucuguu (SEQ ID NO: 4022), UAGgucagag (SEQ ID NO: 4023), AAGguguguu (SEQ ID NO: 406), CUGgucagug (SEQ ID NO: 4024), AAGgugggac (SEQ ID NO: 4025), GUGguaguag (SEQ ID NO: 4026), CUAguuuagg (SEQ ID NO: 4027), CCCgccccau (SEQ ID NO: 4028), GCUguacugc (SEQ ID NO: 4029), GAGguaauau (SEQ ID NO: 1897), UAGguuggug (SEQ ID NO: 4030), AAGguccaac (SEQ ID NO: 4031), UAGgugagga (SEQ ID NO: 2593), GUGguaaguu (SEQ ID NO: 2354), AGUgugagag (SEQ ID NO: 831), AAUguacaug (SEQ ID NO: 497), UUGgcaggug (SEQ ID NO: 4032), UAGguuauug (SEQ ID NO: 4033), CAGguacuga (SEQ ID NO: 1191), GCGguggguc (SEQ ID NO: 4034), UGUguaagau (SEQ ID NO: 4035), GAGgugagua (SEQ ID NO: 2025), GCAgccccgg (SEQ ID NO: 4036), CAGgugcuaa (SEQ ID NO: 4037), AGUguaagag (SEQ ID NO: 815), CAGguacauc (SEQ ID NO: 4038), CAGgugggac (SEQ ID NO: 1403), AGGguaaaua (SEQ ID NO: 727), UAAguaauua (SEQ ID NO: 4039), CAGguaaccg (SEQ ID NO: 1132), AAGguuugca (SEQ ID NO: 461), UAGgugguuu (SEQ ID NO: 4040), CAGgugaccg (SEQ ID NO: 1327), UGUguaagcu (SEQ ID NO: 4041), GGAgugaguc (SEQ ID NO: 2227), AGGguaggag (SEQ ID NO: 752), AGGgugggug (SEQ ID NO: 802), AAGgucugag (SEQ ID NO: 313), GAUguaauau (SEQ ID NO: 4042), GGGguaauua (SEQ ID NO: 4043), UAGguaggua (SEQ ID NO: 2524), GAGgcaagua (SEQ ID NO: 1858), GAGguaagga (SEQ ID NO: 1889), UAGguacuac (SEQ ID NO: 4044), UCGgugggug (SEQ ID NO: 4045), AAGgugugga (SEQ ID NO: 401), CAGgucugcc (SEQ ID NO: 1305), UAAgugagcc (SEQ ID NO: 4046), GAAguaaguu (SEQ ID NO: 1820), GAAguaagcc (SEQ ID NO: 1815), UAGgugcgac (SEQ ID NO: 4047), GAGguauggc (SEQ ID NO: 4048), GCAguaagaa (SEQ ID NO: 2145), CAGgugugga (SEQ ID NO: 1438), UUGguaacgu (SEQ ID NO: 4049), GCUguaaaaa (SEQ ID NO: 4050), UUGguuagua (SEQ ID NO: 4051), AUAguaaggg (SEQ ID NO: 4052), UUGguacuag (SEQ ID NO: 4053), CGGgcagccg (SEQ ID NO: 4054), CAGgugcugg (SEQ ID NO: 1389), UAUgugaguu (SEQ ID NO: 2673), CAGgucuggg (SEQ ID NO: 4055), UAAguaagaa (SEQ ID NO: 2415), AAGguuauua (SEQ ID NO: 4056), AGAguaaagc (SEQ ID NO: 4057), AGAgugugag (SEQ ID NO: 4058), UAGgugcgag (SEQ ID NO: 4059), CAAguaaacg (SEQ ID NO: 4060), AAGguacgua (SEQ ID NO: 4061), CUGgugagua (SEQ ID NO: 1759), CCAguaugua (SEQ ID NO: 4062), UUGgugagug (SEQ ID NO: 3006), UGAguaagua (SEQ ID NO: 2772), GAGguuagca (SEQ ID NO: 4063), GUGguaagcc (SEQ ID NO: 4064), CUGguauggc (SEQ ID NO: 1734), AAAguaacac (SEQ ID NO: 8), CAGguacuaa (SEQ ID NO: 1186), UCUguaaguu (SEQ ID NO: 2747), GAGgugaggg (SEQ ID NO: 2024), ACUgugggua (SEQ ID NO: 647), GAUguuugug (SEQ ID NO: 4065), CAGgugucaa (SEQ ID NO: 4066), CAGgucacca (SEQ ID NO: 4067), CCGgugagua (SEQ ID NO: 4068), UUGguaaaua (SEQ ID NO: 4069), CAGguggggg (SEQ ID NO: 1411), ACUgcaggug (SEQ ID NO: 4070), UAGguauguu (SEQ ID NO: 2554), GGAgcaagug (SEQ ID NO: 4071), UCGgugccuc (SEQ ID NO: 4072), CAAguaacuu (SEQ ID NO: 4073), GAGguaacca (SEQ ID NO: 1879), CAGguaauau (SEQ ID NO: 1151), GGAguaagaa (SEQ ID NO: 4074), GAGguaccuu (SEQ ID NO: 1914), AGGguaagga (SEQ ID NO: 737), CCUgugaguc (SEQ ID NO: 1609), GAGguaaugg (SEQ ID NO: 1900), AUGguguguc (SEQ ID NO: 4075), GGGgugagua (SEQ ID NO: 4076), AGGgucaggu (SEQ ID NO: 4077), UGGguaaggg (SEQ ID NO: 2839), AGGguagguu (SEQ ID NO: 759), AUAgugaguu (SEQ ID NO: 4078), CCCguaggcu (SEQ ID NO: 4079), ACAguaugua (SEQ ID NO: 553), GACgugugua (SEQ ID NO: 4080), GCGgugagga (SEQ ID NO: 4081), CAGgugaccc (SEQ ID NO: 1326), UAAguuuagu (SEQ ID NO: 4082), ACAguugagu (SEQ ID NO: 570), CGGgugaggg (SEQ ID NO: 1639), CAGguggauu (SEQ ID NO: 1398), CGGguagagg (SEQ ID NO: 4083), UAGgugcgug (SEQ ID NO: 2608), GGGguaagaa (SEQ ID NO: 2243), GAGguggggu (SEQ ID NO: 4084), CACguggguu (SEQ ID NO: 4085), ACGguaauug (SEQ ID NO: 4086), AGAgugaguc (SEQ ID NO: 705), UUGgcuccaa (SEQ ID NO: 4087), AAGgugaugc (SEQ ID NO: 355), AAGguugguc (SEQ ID NO: 448), AGCguaaguu (SEQ ID NO: 4088), AUUguaugua (SEQ ID NO: 1006), UCAguuaagu (SEQ ID NO: 4089), CAAguacgug (SEQ ID NO: 4090), CAGgugcgug (SEQ ID NO: 1382), CAGguaggua (SEQ ID NO: 1220), AUGguggggu (SEQ ID NO: 4091), AUGgugaguu (SEQ ID NO: 964), CAGguaauca (SEQ ID NO: 4092), AAGguagggu (SEQ ID NO: 226), CAGgccaagg (SEQ ID NO: 4093), GUGgugagag (SEQ ID NO: 4094), AAGguuggug (SEQ ID NO: 449), CAGguacucu (SEQ ID NO: 1190), UAGgcaugug (SEQ ID NO: 4095), UUGguaccuu (SEQ ID NO: 4096), CUGgugugcc (SEQ ID NO: 4097), ACAguugcca (SEQ ID NO: 4098), UUGguaauau (SEQ ID NO: 4099), GAGgugcaug (SEQ ID NO: 4100), UUGguuugua (SEQ ID NO: 3028), UUGguaagug (SEQ ID NO: 2963), UGUgugugug (SEQ ID NO: 4101), GUGguuugua (SEQ ID NO: 2398), GCGguacaca (SEQ ID NO: 4102), AGAguaugcu (SEQ ID NO: 4103), UUUguaagua (SEQ ID NO: 3038), UCUgugcggg (SEQ ID NO: 4104), AAGgucagug (SEQ ID NO: 295), GAGguaggaa (SEQ ID NO: 1930), GCGguuagca (SEQ ID NO: 4105), AGGgugaggg (SEQ ID NO: 793), GAAgugagua (SEQ ID NO: 4106), CAGgugacag (SEQ ID NO: 4107), AAGgugauua (SEQ ID NO: 357), GAGgccagcc (SEQ ID NO: 4108), GAGgucuccu (SEQ ID NO: 4109), UAGguauuac (SEQ ID NO: 2556), CAUguaagag (SEQ ID NO: 1519), CUGguagggc (SEQ ID NO: 4110), GAAguaagua (SEQ ID NO: 1818), CGGguaagug (SEQ ID NO: 4111), CAGguaaucu (SEQ ID NO: 4112), GUGguaggua (SEQ ID NO: 4113), CAGgugggua (SEQ ID NO: 1413), AAGgccagug (SEQ ID NO: 4114), AAAgugaauc (SEQ ID NO: 4115), ACGguuacgu (SEQ ID NO: 4116), AUGguaggaa (SEQ ID NO: 917), CGGgugagac (SEQ ID NO: 4117), GAGguuggaa (SEQ ID NO: 2099), UGGgugagcc (SEQ ID NO: 2871), CCAgugagua (SEQ ID NO: 1564), CUAguacgag (SEQ ID NO: 4118), CAGguaugac (SEQ ID NO: 1248), GCUgugaggu (SEQ ID NO: 4119), CUGguaugaa (SEQ ID NO: 4120), GGUguacgac (SEQ ID NO: 4121), CUUgugagug (SEQ ID NO: 4122), GUGgugagca (SEQ ID NO: 2380), CUGguaacuu (SEQ ID NO: 1696), CAGguacuau (SEQ ID NO: 1188), AGGguaaggg (SEQ ID NO: 739), UUGguuaguu (SEQ ID NO: 3025), GGUguaagca (SEQ ID NO: 2302), UCGgugagga (SEQ ID NO: 4123), UGGguaaaca (SEQ ID NO: 4124), UCGguacgug (SEQ ID NO: 4125), UAGguagcag (SEQ ID NO: 4126), CUGguaaggc (SEQ ID NO: 1704), GUGguaagga (SEQ ID NO: 2349), UAAguaagca (SEQ ID NO: 2418), GAGguuccaa (SEQ ID NO: 4127), CUGguaugga (SEQ ID NO: 4128), GGGgugggua (SEQ ID NO: 2288), CAGguuuccc (SEQ ID NO: 4129), CAGgucucug (SEQ ID NO: 4130), GAGgugagga (SEQ ID NO: 2022), CUUguggguu (SEQ ID NO: 1805), AUGgugagac (SEQ ID NO: 953), CAGgugaagg (SEQ ID NO: 1319), GCGguagggg (SEQ ID NO: 4131), GUUguuuccc (SEQ ID NO: 4132), AAAgcaucca (SEQ ID NO: 4133), GUGguagguu (SEQ ID NO: 2367), AAGgugugaa (SEQ ID NO: 398), CAGguacagu (SEQ ID NO: 1167), AAGguaccaa (SEQ ID NO: 182), UUGguaauug (SEQ ID NO: 2969), AAGgugcuca (SEQ ID NO: 4134), AAGguucaac (SEQ ID NO: 4135), CAGguuuaca (SEQ ID NO: 4136), GCUguaagug (SEQ ID NO: 2195), AGGguauguc (SEQ ID NO: 769), GAGgucgggg (SEQ ID NO: 1996), AAGgugccug (SEQ ID NO: 363), AAGguaaaaa (SEQ ID NO: 119), GUGgugaguu (SEQ ID NO: 2385), UAGguaagaa (SEQ ID NO: 4137), AGGguauccu (SEQ ID NO: 4138), GUGguaauau (SEQ ID NO: 4139), UCUguaagua (SEQ ID NO: 2744), UGGguaugga (SEQ ID NO: 4140), AUGguaugga (SEQ ID NO: 935), GACgugagcc (SEQ ID NO: 1854), CUGguuuggc (SEQ ID NO: 4141), AUGguauauc (SEQ ID NO: 4142), AAAguaaacu (SEQ ID NO: 4143), AGCgugagug (SEQ ID NO: 721), CUGguauaga (SEQ ID NO: 4144), CAGgugggga (SEQ ID NO: 1409), AGAguauguu (SEQ ID NO: 696), UAGguacuug (SEQ ID NO: 4145), GCAguaggug (SEQ ID NO: 4146), AGUguauguc (SEQ ID NO: 4147), AAGguuaagc (SEQ ID NO: 413), CUGguggccu (SEQ ID NO: 4148), GAAgugaguc (SEQ ID NO: 1839), UUGguguaag (SEQ ID NO: 4149), CAGguaagaa (SEQ ID NO: 1138), CGGgucucgg (SEQ ID NO: 4150), GAGgugcaca (SEQ ID NO: 2035), CUCguuaguu (SEQ ID NO: 4151), AAGgugauca (SEQ ID NO: 352), UAUguaagaa (SEQ ID NO: 2649), GAGgugcuug (SEQ ID NO: 2047), CAGgugguca (SEQ ID NO: 4152), ACGguaaguc (SEQ ID NO: 4153), ACAguaaugu (SEQ ID NO: 4154), CCUguaaggu (SEQ ID NO: 4155), GAGguuaagu (SEQ ID NO: 4156), UCGguaugug (SEQ ID NO: 2725), UGGguauguu (SEQ ID NO: 2863), AAGguauuac (SEQ ID NO: 268), CAGgugaggg (SEQ ID NO: 1343), UUGguaaaca (SEQ ID NO: 4157), AAGguagugu (SEQ ID NO: 4158), GAGguguggc (SEQ ID NO: 4159), CAGguacgga (SEQ ID NO: 4160), AAGgucauca (SEQ ID NO: 4161), CAAguaggca (SEQ ID NO: 4162), CAGgugaaac (SEQ ID NO: 4163), CAGguacugc (SEQ ID NO: 1192), AAUgcaagug (SEQ ID NO: 4164), CAUguaauuc (SEQ ID NO: 4165), AAGguaugcu (SEQ ID NO: 259), CUGgugaguu (SEQ ID NO: 1762), CAGgugguuu (SEQ ID NO: 4166), UGUgugagua (SEQ ID NO: 2922), AAGgucggug (SEQ ID NO: 4167), AUGguaaauu (SEQ ID NO: 883), AGGguauuac (SEQ ID NO: 771), AGUguaugga (SEQ ID NO: 4168), AACguaagau (SEQ ID NO: 4169), GUGguaaggu (SEQ ID NO: 4170), ACUguuagua (SEQ ID NO: 4171), CAGguaucag (SEQ ID NO: 1239), AAGguuaguu (SEQ ID NO: 425), CUGgugagcu (SEQ ID NO: 1754), UUGgugagcu (SEQ ID NO: 4172), UGUguacgua (SEQ ID NO: 4173), GAGgucagcc (SEQ ID NO: 4174), GAGguagaau (SEQ ID NO: 4175), AAGguaugag (SEQ ID NO: 255), UAGguauuuc (SEQ ID NO: 2563), UGUguaacac (SEQ ID NO: 4176), AGUguaaggc (SEQ ID NO: 4177), GAGgucugcu (SEQ ID NO: 4178), AAGguuagca (SEQ ID NO: 418), CAGguaaaug (SEQ ID NO: 1127), AACguaagcu (SEQ ID NO: 4179), CAGgucugca (SEQ ID NO: 4180), CAGguauugu (SEQ ID NO: 1267), GUGguaauuc (SEQ ID NO: 2356), GAGguauaug (SEQ ID NO: 1951), GCCgugagcc (SEQ ID NO: 4181), GAGguaagag (SEQ ID NO: 1883), UGAguaugua (SEQ ID NO: 2787), CAGguaaggg (SEQ ID NO: 1145), GAGguaaauu (SEQ ID NO: 1876), CAGgcaacuu (SEQ ID NO: 4182), UGUguaaguc (SEQ ID NO: 2908), CAGgugcgcu (SEQ ID NO: 4183), CGGguaaacc (SEQ ID NO: 4184), CCGgucaguc (SEQ ID NO: 4185), UAGgugggcg (SEQ ID NO: 4186), GCGgucaguu (SEQ ID NO: 4187), GGGguggguc (SEQ ID NO: 4188), AGCguaauag (SEQ ID NO: 4189), ACGgugaguc (SEQ ID NO: 4190), CUGguacuug (SEQ ID NO: 1722), CAGguuggua (SEQ ID NO: 4191), AGAguaugug (SEQ ID NO: 695), CUGgugggua (SEQ ID NO: 1771), GAGguggcuu (SEQ ID NO: 4192), AUAguauuga (SEQ ID NO: 4193), UGAgucguce (SEQ ID NO: 4194), CAGgugcucu (SEQ ID NO: 4195), UACguaauau (SEQ ID NO: 4196), GCUguccuga (SEQ ID NO: 4197), CAGgcugcac (SEQ ID NO: 4198), CUGgugcgcu (SEQ ID NO: 1766), GCGguaagaa (SEQ ID NO: 4199), UAAguuacuu (SEQ ID NO: 4200), GAAgugagug (SEQ ID NO: 1840), UAGgcaaguc (SEQ ID NO: 2460), UAAguaaaua (SEQ ID NO: 4201), ACGgugagug (SEQ ID NO: 607), CAGguagguu (SEQ ID NO: 1223), GGGguauaac (SEQ ID NO: 4202), GUUgugaguu (SEQ ID NO: 2410), CAUgugagua (SEQ ID NO: 1539), GAGgugcauu (SEQ ID NO: 4203), AAGguuugua (SEQ ID NO: 466), UCGguaaugu (SEQ ID NO: 4204), CGAguaaggg (SEQ ID NO: 1616), GAGgcacgga (SEQ ID NO: 4205), AGGgugugga (SEQ ID NO: 4206), CAGguauggu (SEQ ID NO: 1257), AAGguagaaa (SEQ ID NO: 203), CAGgugccug (SEQ ID NO: 1373), UGGguauaug (SEQ ID NO: 4207), UGAgugagac (SEQ ID NO: 4208), UGGguaauuu (SEQ ID NO: 2847), AUGguaaaua (SEQ ID NO: 881), AAGgcaaagg (SEQ ID NO: 4209), AGUguuuguu (SEQ ID NO: 4210), AUGguauugg (SEQ ID NO: 4211), CUGgugaggc (SEQ ID NO: 1756), UUGguaaaau (SEQ ID NO: 2948), ACAgugaguu (SEQ ID NO: 563), CAGgugcugu (SEQ ID NO: 4212), GAGguuaaga (SEQ ID NO: 2080), AGAguaagaa (SEQ ID NO: 659), GAGguccgcg (SEQ ID NO: 4213), GUGgugagga (SEQ ID NO: 2382), CAGgugagcc (SEQ ID NO: 1338), CAGgugacau (SEQ ID NO: 1324), AUGgcaagcu (SEQ ID NO: 4214), UCGguaauau (SEQ ID NO: 4215), CAGgcaacaa (SEQ ID NO: 4216), GGGguaggga (SEQ ID NO: 2257), CUGgucucge (SEQ ID NO: 4217), UAGguaacga (SEQ ID NO: 4218), CGGguaaggu (SEQ ID NO: 4219), UAGguaaugc (SEQ ID NO: 4220), CAGgcaagaa (SEQ ID NO: 1099), ACAguaggua (SEQ ID NO: 4221), CAAguaugag (SEQ ID NO: 1049), GCUguucgaa (SEQ ID NO: 4222), AAGguuaugc (SEQ ID NO: 4223), GAUgugaguu (SEQ ID NO: 2136), CAGguggaga (SEQ ID NO: 1396), AGAguuaguu (SEQ ID NO: 4224), UGAgugugeg (SEQ ID NO: 4225), GAGguacagc (SEQ ID NO: 1907), CAGguaagac (SEQ ID NO: 1139), CAUgugcuuu (SEQ ID NO: 4226), AGGguguguu (SEQ ID NO: 4227), ACAguuaagg (SEQ ID NO: 4228), ACAgugaggg (SEQ ID NO: 4229), GAUguauacc (SEQ ID NO: 4230), UUAguaagcu (SEQ ID NO: 4231), CAGguaagau (SEQ ID NO: 1141), AGAgcugcgu (SEQ ID NO: 4232), GAGgcaaguu (SEQ ID NO: 1860), GAAguaagug (SEQ ID NO: 1819), AAGgugaaaa (SEQ ID NO: 4233), AAGguaccua (SEQ ID NO: 4234), GAGguaucag (SEQ ID NO: 4235), AUGguaugua (SEQ ID NO: 4236), AAGguaugaa (SEQ ID NO: 253), UUGgugagcc (SEQ ID NO: 4237), AAGguuagga (SEQ ID NO: 420), AGGguaugua (SEQ ID NO: 768), CAGguaccga (SEQ ID NO: 4238), AGAguaaacu (SEQ ID NO: 4239), AAGgugcaua (SEQ ID NO: 4240), AAGguaaugu (SEQ ID NO: 167), CCGgugugug (SEQ ID NO: 4241), AGGguaaauu (SEQ ID NO: 729), GGGguuuggc (SEQ ID NO: 4242), CAGguacacg (SEQ ID NO: 1164), UUGguaacca (SEQ ID NO: 4243), GAGgucaggu (SEQ ID NO: 1986), UCUguuggua (SEQ ID NO: 4244), CAGguuaguu (SEQ ID NO: 1458), UUGguauguc (SEQ ID NO: 4245), AAGgugcguc (SEQ ID NO: 4246), AGGguaagaa (SEQ ID NO: 733), UUUguaagcc (SEQ ID NO: 4247), AAGgucaggu (SEQ ID NO: 292), CUGguaaacu (SEQ ID NO: 4248), UCGguaauuu (SEQ ID NO: 4249), CUGguaggcu (SEQ ID NO: 4250), GAGgucugua (SEQ ID NO: 4251), GAGguacuuu (SEQ ID NO: 1922), CUGguaaagg (SEQ ID NO: 4252), CGGgugugug (SEQ ID NO: 1650), CAGguguggu (SEQ ID NO: 4253), UCGguacguc (SEQ ID NO: 4254), CAGgugccag (SEQ ID NO: 4255), GGGgugagaa (SEQ ID NO: 2275), ACAgcuagua (SEQ ID NO: 4256), AAGguauagc (SEQ ID NO: 4257), CUGguaggag (SEQ ID NO: 4258), GCUguacgua (SEQ ID NO: 4259), AAGguaaagg (SEQ ID NO: 128), CAAgcacgag (SEQ ID NO: 4260), CUAguaagac (SEQ ID NO: 4261), CCCguaagcg (SEQ ID NO: 4262), CAAgugugag (SEQ ID NO: 1078), AUGguaaggg (SEQ ID NO: 897), AAGgugaggg (SEQ ID NO: 345), CAAguaggua (SEQ ID NO: 1041), GGUguugcug (SEQ ID NO: 2321), GAGguacugu (SEQ ID NO: 1920), UAGguaagau (SEQ ID NO: 2484), CAGgugcgaa (SEQ ID NO: 1374), GAGguccagg (SEQ ID NO: 4263), UUGguauaca (SEQ ID NO: 2982), GGAgugagua (SEQ ID NO: 2226), GAGgugagau (SEQ ID NO: 2017), AAGguggggc (SEQ ID NO: 4264), CAGguaaacg (SEQ ID NO: 4265), UCGguaacuu (SEQ ID NO: 4266), CAGguaaauu (SEQ ID NO: 1128), GAGgugcgca (SEQ ID NO: 4267), ACUgugagua (SEQ ID NO: 643), ACGgugugac (SEQ ID NO: 4268), GUGguaaguc (SEQ ID NO: 2352), CAGguaggca (SEQ ID NO: 1215), CAGgucagca (SEQ ID NO: 1277), GUGguaugug (SEQ ID NO: 4269), AAAguaucug (SEQ ID NO: 4270), CGGguaugua (SEQ ID NO: 4271), AAGguaauaa (SEQ ID NO: 157), GAGgugggga (SEQ ID NO: 2060), GCUguaggug (SEQ ID NO: 2197), GAAgugaguu (SEQ ID NO: 1841), AAAguauuua (SEQ ID NO: 4272), UAUguaagua (SEQ ID NO: 2653), ACGguaugag (SEQ ID NO: 4273), CUGgugagug (SEQ ID NO: 1761), AGAguaaaau (SEQ ID NO: 4274), GCUguauggc (SEQ ID NO: 4275), AUGguaaacc (SEQ ID NO: 879), GCAguaauaa (SEQ ID NO: 4276), UAAguauuua (SEQ ID NO: 4277), AAUgucagug (SEQ ID NO: 515), AUUgcaggag (SEQ ID NO: 4278), CCGguaagaa (SEQ ID NO: 4279), AAGgcaaguu (SEQ ID NO: 101), GAGguuuguc (SEQ ID NO: 4280), AAGguaacug (SEQ ID NO: 139), AAAguaugag (SEQ ID NO: 4281), GAUguuagua (SEQ ID NO: 4282), CAGguggguc (SEQ ID NO: 1414), AAGguaccga (SEQ ID NO: 4283), CCAguaauua (SEQ ID NO: 4284), GUGguaugcg (SEQ ID NO: 4285), AUGgugcgcu (SEQ ID NO: 4286), CAGgucuaug (SEQ ID NO: 4287), AAGguauuua (SEQ ID NO: 274), CUAguaagau (SEQ ID NO: 4288), AGAguaauuu (SEQ ID NO: 675), GAGguaacgu (SEQ ID NO: 4289), AAGguagcca (SEQ ID NO: 212), CUGgucccgg (SEQ ID NO: 4290), GAGguccuuc (SEQ ID NO: 4291), ACGgucaccc (SEQ ID NO: 4292), AAGguaauac (SEQ ID NO: 158), CAGgugcaug (SEQ ID NO: 1367), AUGguaauag (SEQ ID NO: 4293), UUUguaacac (SEQ ID NO: 4294), UGGguaugau (SEQ ID NO: 4295), CAGgcccccc (SEQ ID NO: 4296), AGAguaguaa (SEQ ID NO: 4297), AGUguaagaa (SEQ ID NO: 814), GAAguauguu (SEQ ID NO: 1833), CAGgugugca (SEQ ID NO: 1434), UUGgugaggg (SEQ ID NO: 3003), UGGguugguu (SEQ ID NO: 4298), CAGguacgua (SEQ ID NO: 1184), GAGgugcggc (SEQ ID NO: 4299), UCUguacggg (SEQ ID NO: 4300), CGGgugcgug (SEQ ID NO: 4301), UACguaagug (SEQ ID NO: 2455), CAUguaagga (SEQ ID NO: 4302), CAGgugacgg (SEQ ID NO: 1329), GAUguaugcu (SEQ ID NO: 4303), UCUgcaauuc (SEQ ID NO: 4304), UGAguaaggc (SEQ ID NO: 2770), GAGguauauu (SEQ ID NO: 1952), AGAgugaguu (SEQ ID NO: 707), AAGguaagcu (SEQ ID NO: 148), UAGgugaagu (SEQ ID NO: 2580), CAGguuagua (SEQ ID NO: 1455), UAUguaagug (SEQ ID NO: 2655), UUGguggggg (SEQ ID NO: 4305), UGAgcucaaa (SEQ ID NO: 4306), UCGguaugua (SEQ ID NO: 4307), UAAguaugcc (SEQ ID NO: 4308), AAUguaagua (SEQ ID NO: 489), CAGguuugca (SEQ ID NO: 4309), ACGgugagag (SEQ ID NO: 4310), CAGguguuuu (SEQ ID NO: 4311), GUGgugagcc (SEQ ID NO: 4312), AGGguacaua (SEQ ID NO: 4313), UAGguaaccc (SEQ ID NO: 4314), GUGgucagua (SEQ ID NO: 4315), CUGgugagcc (SEQ ID NO: 4316), CAGgugcuua (SEQ ID NO: 1390), AUAgucguga (SEQ ID NO: 4317), AUAgugagug (SEQ ID NO: 862), GAGgucaaaa (SEQ ID NO: 4318), CGUguagcuu (SEQ ID NO: 4319), CAGguguuug (SEQ ID NO: 4320), CAGguuggac (SEQ ID NO: 4321), CAGguaagcu (SEQ ID NO: 4322), AGGgucagaa (SEQ ID NO: 4323), CACguauguc (SEQ ID NO: 4324), CACgugagug (SEQ ID NO: 1098), GGGguacgga (SEQ ID NO: 4325), AAGgcaggac (SEQ ID NO: 4326), GAGgugaagc (SEQ ID NO: 4327), GAGguuugaa (SEQ ID NO: 4328), CAGguaagug (SEQ ID NO: 1148), CAGguaacca (SEQ ID NO: 1131), CAGguacucc (SEQ ID NO: 1189), AAGgugcuuu (SEQ ID NO: 371), GAGguaaaua (SEQ ID NO: 1873), GAGgcaggug (SEQ ID NO: 4329), GAGguucgga (SEQ ID NO: 4330), CAGguauuug (SEQ ID NO: 1270), CAGguaaaua (SEQ ID NO: 1125), CAGgugaugu (SEQ ID NO: 1354), CAGgugauac (SEQ ID NO: 4331), GAGgugaggc (SEQ ID NO: 2023), AGGguggggg (SEQ ID NO: 4332), UAAguaaguu (SEQ ID NO: 2425), UGGgugaaca (SEQ ID NO: 4333), UAGguacugc (SEQ ID NO: 4334), CAGgcuccug (SEQ ID NO: 4335), AGGguaggca (SEQ ID NO: 753), CAGgugcccg (SEQ ID NO: 1371), GAGguacauc (SEQ ID NO: 4336), AGGgugugug (SEQ ID NO: 804), AAGguaguaa (SEQ ID NO: 231), UGGguaugag (SEQ ID NO: 2859), GGGgugugug (SEQ ID NO: 2294), CUAguaggug (SEQ ID NO: 4337), GAGgcaagga (SEQ ID NO: 4338), AAGgcaagac (SEQ ID NO: 4339), AAAgugcggu (SEQ ID NO: 4340), AAGguugguu (SEQ ID NO: 450), GAGguuaaug (SEQ ID NO: 4341), UUGgugaguc (SEQ ID NO: 3005), UCGguuagcu (SEQ ID NO: 2738), GCAguaagca (SEQ ID NO: 4342), AAGgcaagca (SEQ ID NO: 4343), ACAguaagcu (SEQ ID NO: 4344), GAGguaacag (SEQ ID NO: 1878), AAAguacgua (SEQ ID NO: 4345), GAGguaauac (SEQ ID NO: 1896), UUGguaggug (SEQ ID NO: 2980), CUGguuaguc (SEQ ID NO: 4346), GAGgugacgc (SEQ ID NO: 4347), ACAguaagga (SEQ ID NO: 4348), AAUguacuua (SEQ ID NO: 4349), GGGguacagu (SEQ ID NO: 4350), CGUguaugug (SEQ ID NO: 4351), UCCguagguu (SEQ ID NO: 4352), GAGguggucg (SEQ ID NO: 4353), UCAgugaguc (SEQ ID NO: 4354), AAAguaagca (SEQ ID NO: 15), GAGgucuggu (SEQ ID NO: 1999), GAGguaauua (SEQ ID NO: 4355), GUAguaagua (SEQ ID NO: 2323), AAGgugggga (SEQ ID NO: 382), UCUgugagca (SEQ ID NO: 4356), GAAguucgug (SEQ ID NO: 4357), ACGgugaggc (SEQ ID NO: 4358), UCAgugagua (SEQ ID NO: 2699), UAGguaguug (SEQ ID NO: 4359), GGUgucuggg (SEQ ID NO: 4360), GGGguaagug (SEQ ID NO: 2252), GAGguggguu (SEQ ID NO: 2066), UGUgugaguu (SEQ ID NO: 4361), CAUguaagua (SEQ ID NO: 1522), AAGguaggug (SEQ ID NO: 229), AAUguaggag (SEQ ID NO: 4362), GAGgcacguc (SEQ ID NO: 4363), CAAguacauu (SEQ ID NO: 4364), UUGguacaga (SEQ ID NO: 4365), GAGguaguag (SEQ ID NO: 1941), AAAgugaggg (SEQ ID NO: 57), UUGgucagug (SEQ ID NO: 4366), AGGgugaguc (SEQ ID NO: 796), CAGgugaaca (SEQ ID NO: 1317), GGUgugggcc (SEQ ID NO: 4367), CGGgugagcu (SEQ ID NO: 4368), GGGgugaguc (SEQ ID NO: 2283), ACAgugagag (SEQ ID NO: 4369), AGGgugaggu (SEQ ID NO: 794), GCUguaaguc (SEQ ID NO: 2194), AUAguagguu (SEQ ID NO: 4370), CAGgcaugug (SEQ ID NO: 1114), AAGguaaguu (SEQ ID NO: 156), CAGguccgug (SEQ ID NO: 4371), GAGgcaggua (SEQ ID NO: 4372), AUGguggaag (SEQ ID NO: 4373), AUGgugggcg (SEQ ID NO: 4374), GAGgugagaa (SEQ ID NO: 2014), AGUgugagca (SEQ ID NO: 832), UUGguaagua (SEQ ID NO: 2962), CAAguaagca (SEQ ID NO: 4375), GGUgugagcu (SEQ ID NO: 2313), CCCgugggua (SEQ ID NO: 4376), CAGguagaau (SEQ ID NO: 4377), CAGgcugagc (SEQ ID NO: 4378), CUGguggccc (SEQ ID NO: 4379), UGAguaagag (SEQ ID NO: 4380), CACguuagcu (SEQ ID NO: 4381), AAGgugaguc (SEQ ID NO: 348), AAGguagcuc (SEQ ID NO: 4382), UCGgugaguu (SEQ ID NO: 4383), GAGgcccuuc (SEQ ID NO: 4384), CAGguuaugc (SEQ ID NO: 4385), CCUguaagcu (SEQ ID NO: 4386), CAGgucuccu (SEQ ID NO: 4387), UAGguaggcu (SEQ ID NO: 4388), GGGguagggg (SEQ ID NO: 4389), AAGguaguga (SEQ ID NO: 4390), GAGguuguug (SEQ ID NO: 4391), CAGguugguu (SEQ ID NO: 1489), AAAguaagcc (SEQ ID NO: 16), ACAgugagug (SEQ ID NO: 562), UGGgugugau (SEQ ID NO: 4392), CCCguaacua (SEQ ID NO: 4393), AAGguguugc (SEQ ID NO: 408), AAAgcuggug (SEQ ID NO: 4394), GAGguauagu (SEQ ID NO: 4395), ACGguaagag (SEQ ID NO: 4396), AUGguacggu (SEQ ID NO: 913), GAGgccaguu (SEQ ID NO: 4397), GAGguaugcg (SEQ ID NO: 1960), UCGgugggag (SEQ ID NO: 4398), AAGguggaua (SEQ ID NO: 372), CCAguguggc (SEQ ID NO: 4399), AGGguaagug (SEQ ID NO: 742), UCUguagguc (SEQ ID NO: 4400), CAGgcaagga (SEQ ID NO: 1102), CGGguaauuu (SEQ ID NO: 1628), AUUgugaguc (SEQ ID NO: 1010), CAGguaaacc (SEQ ID NO: 1121), AAGgucaauu (SEQ ID NO: 4401), AAGgugaaua (SEQ ID NO: 327), GUCguaagaa (SEQ ID NO: 4402), GCGguaaguc (SEQ ID NO: 4403), CUGguagage (SEQ ID NO: 4404), GAGgucgguc (SEQ ID NO: 4405), CAGguaaaca (SEQ ID NO: 1120), AAGgcaagga (SEQ ID NO: 98), CAGgucgucu (SEQ ID NO: 4406), GGGguagggc (SEQ ID NO: 4407), CUGguacuaa (SEQ ID NO: 1721), GAGguagcug (SEQ ID NO: 1929), CUUgucagcu (SEQ ID NO: 4408), UAGguaaggc (SEQ ID NO: 2489), CUGguauuac (SEQ ID NO: 4409), UAAguacguc (SEQ ID NO: 4410), AAGguaagcc (SEQ ID NO: 146), ACGgugaaag (SEQ ID NO: 4411), CCAgccaaua (SEQ ID NO: 4412), CAGguuuguc (SEQ ID NO: 4413), AAGguauaau (SEQ ID NO: 239), AAGgucuuag (SEQ ID NO: 4414), AGGgugagcu (SEQ ID NO: 791), AAGguuaggg (SEQ ID NO: 4415), CGGguaaauu (SEQ ID NO: 4416), CAGguaacgg (SEQ ID NO: 4417), AGAgugugua (SEQ ID NO: 4418), ACAguaaguu (SEQ ID NO: 549), GAUguaauuu (SEQ ID NO: 4419), GAGguaggga (SEQ ID NO: 1934), UUGgcaagug (SEQ ID NO: 2945), AAAgugagga (SEQ ID NO: 4420), AAGguagugc (SEQ ID NO: 234), AGAguaauuc (SEQ ID NO: 674), GGAguaaaua (SEQ ID NO: 4421), GUGguaccca (SEQ ID NO: 4422), CAGguauugc (SEQ ID NO: 4423), GAUgugaggg (SEQ ID NO: 4424), CAAguaaauc (SEQ ID NO: 1017), CAGgugucuc (SEQ ID NO: 1428), AAGguaacag (SEQ ID NO: 4425), UUGguaaaag (SEQ ID NO: 4426), CAGguaucau (SEQ ID NO: 1240), ACGgugagac (SEQ ID NO: 4427), CUGguaugac (SEQ ID NO: 4428), CAGguucacu (SEQ ID NO: 4429), GAGgugauca (SEQ ID NO: 4430), AGUguaaguc (SEQ ID NO: 4431), AACguaagua (SEQ ID NO: 4432), AAAgugagug (SEQ ID NO: 60), GAGguacagg (SEQ ID NO: 4433), CAAguaauga (SEQ ID NO: 4434), GAUguaagga (SEQ ID NO: 4435), UCAguucccc (SEQ ID NO: 4436), GCGguaagga (SEQ ID NO: 4437), UAGguacuaa (SEQ ID NO: 4438), AAGgugaaag (SEQ ID NO: 321), ACUguaagug (SEQ ID NO: 4439), UGGguaugug (SEQ ID NO: 2862), AUGguaacag (SEQ ID NO: 884), CAGguagggu (SEQ ID NO: 1219), ACAguaagug (SEQ ID NO: 548), AAGgugcucc (SEQ ID NO: 366), AAGgugugcu (SEQ ID NO: 4440), AAGgugguga (SEQ ID NO: 4441), ACGgugcgcc (SEQ ID NO: 4442), AAGguauugc (SEQ ID NO: 4443), GGGguaugug (SEQ ID NO: 2267), CAGgugggcu (SEQ ID NO: 1408), GAGguauguu (SEQ ID NO: 1968), AACgugaaua (SEQ ID NO: 4444), CAGguaaugg (SEQ ID NO: 1154), UAGguaugau (SEQ ID NO: 4445), CAGgcaggug (SEQ ID NO: 1108), GGGguugguc (SEQ ID NO: 4446), AAGguauggg (SEQ ID NO: 262), UAAgugaggc (SEQ ID NO: 4447), CAAgugaucg (SEQ ID NO: 4448), AAAguacggg (SEQ ID NO: 4449), AGAgcuacag (SEQ ID NO: 4450), GAGgugggaa (SEQ ID NO: 2054), CAGguacuuu (SEQ ID NO: 1195), GAGgugagag (SEQ ID NO: 2016), CAGguagguc (SEQ ID NO: 1221), UGGguacagc (SEQ ID NO: 4451), AAGgugucag (SEQ ID NO: 396), AAGgcaagaa (SEQ ID NO: 4452), GAGguaaaca (SEQ ID NO: 4453), AAGguaaagu (SEQ ID NO: 129), AAGguaguca (SEQ ID NO: 4454), CUGguauguc (SEQ ID NO: 4455), GAGguauggg (SEQ ID NO: 1963), AAGguauugu (SEQ ID NO: 273), CUGguacuga (SEQ ID NO: 4456), GAGguaagcu (SEQ ID NO: 1888), UGGgugggua (SEQ ID NO: 2883), CAGguucgug (SEQ ID NO: 4457), AAGguauggu (SEQ ID NO: 4458), CAGgugagca (SEQ ID NO: 1337), UGGguaaauu (SEQ ID NO: 2827), UGUguaggug (SEQ ID NO: 4459), UGUgugagcc (SEQ ID NO: 2921), CUGguaauau (SEQ ID NO: 4460), AAAguauguu (SEQ ID NO: 45), UGUguaagaa (SEQ ID NO: 2903), CUAgugagaa (SEQ ID NO: 4461), AGGguagguc (SEQ ID NO: 757), AAGgugggug (SEQ ID NO: 385), UCGguaagug (SEQ ID NO: 4462), AGUguaaaua (SEQ ID NO: 812), GAUguaagug (SEQ ID NO: 2122), AAGguuagug (SEQ ID NO: 424), UAGguaagca (SEQ ID NO: 2485), CAAgugagaa (SEQ ID NO: 1061), AGUguaagua (SEQ ID NO: 819), CAGgugaauc (SEQ ID NO: 1321), UGGgugagac (SEQ ID NO: 2868), AAGguagggc (SEQ ID NO: 224), CUGguuugug (SEQ ID NO: 1788), GCGguagggc (SEQ ID NO: 4463), GAGguaaucc (SEQ ID NO: 4464), AUUguaauaa (SEQ ID NO: 4465), CUGgugaaua (SEQ ID NO: 1748), AAGguuuaaa (SEQ ID NO: 4466), CCUguacugu (SEQ ID NO: 4467), GCGgugagcg (SEQ ID NO: 4468), AAGguaaucc (SEQ ID NO: 162), UAUgugagua (SEQ ID NO: 2671), CCCgugagug (SEQ ID NO: 1573), CAGgugcaga (SEQ ID NO: 1363), CAGgucaguu (SEQ ID NO: 1284), CAGguaggcu (SEQ ID NO: 4469), AAAguaagug (SEQ ID NO: 23), UAGguugguc (SEQ ID NO: 4470), CAGguugccu (SEQ ID NO: 4471), AAGguaugga (SEQ ID NO: 260), GGUguggacg (SEQ ID NO: 4472), AAAgugagaa (SEQ ID NO: 51), AGGgugagag (SEQ ID NO: 788), GAUguggcau (SEQ ID NO: 4473), UCGguaaggu (SEQ ID NO: 4474), GAGgugcguc (SEQ ID NO: 4475), CGGgugaguc (SEQ ID NO: 4476), AAGguacggg (SEQ ID NO: 190), GAGguucuug (SEQ ID NO: 4477), AAGgugcuug (SEQ ID NO: 4478), UAGguaugua (SEQ ID NO: 2551), AUGgucagca (SEQ ID NO: 4479), CGGguacuca (SEQ ID NO: 4480), AGGgugagga (SEQ ID NO: 792), AUCgugagua (SEQ ID NO: 869), UCAguaagua (SEQ ID NO: 2689), UAGguaaaua (SEQ ID NO: 2469), AAGguaauug (SEQ ID NO: 170), GAAgucagug (SEQ ID NO: 1835), CAGguacaaa (SEQ ID NO: 1160), AAAguuaauc (SEQ ID NO: 4481), AGCgugagcg (SEQ ID NO: 4482), CCGgcuggug (SEQ ID NO: 4483), AGUguaauuu (SEQ ID NO: 4484), UGAgccacuc (SEQ ID NO: 4485), GGGgucugua (SEQ ID NO: 4486), AUGgcauguc (SEQ ID NO: 4487), CGGguaaaga (SEQ ID NO: 4488), AGGguagcau (SEQ ID NO: 4489), CGGguaggag (SEQ ID NO: 1631), GAGguucgug (SEQ ID NO: 4490), UAAguuauuc (SEQ ID NO: 4491), UAUguaagau (SEQ ID NO: 2650), AAGguaguuu (SEQ ID NO: 237), CAGgugguau (SEQ ID NO: 4492), GUGguaauga (SEQ ID NO: 2355), AAGgugauuu (SEQ ID NO: 359), CAGgugaagu (SEQ ID NO: 4493), GUAguaauua (SEQ ID NO: 4494), AUGguuggug (SEQ ID NO: 4495), CCAguaagug (SEQ ID NO: 1557), UAGgugagag (SEQ ID NO: 2589), AUGgugaggc (SEQ ID NO: 959), AAAguuagug (SEQ ID NO: 72), AAGgugccuu (SEQ ID NO: 4496), UAGguaugag (SEQ ID NO: 2546), CAGgugugac (SEQ ID NO: 1431), CUGguggguu (SEQ ID NO: 1774), AUGguaagga (SEQ ID NO: 896), UCUguaagaa (SEQ ID NO: 2740), UCCgugaguu (SEQ ID NO: 4497), AAAgcaggua (SEQ ID NO: 4498), UAUgugagug (SEQ ID NO: 2672), CAGguggagg (SEQ ID NO: 4499), CAGguuagac (SEQ ID NO: 4500), AUAguaagac (SEQ ID NO: 846), AAGguguugu (SEQ ID NO: 4501), GAGgucugug (SEQ ID NO: 4502), AAGguaagau (SEQ ID NO: 144), CAUguaaguu (SEQ ID NO: 1524), CUGguaauua (SEQ ID NO: 4503), CAGguaggcg (SEQ ID NO: 4504), AGAguaaguc (SEQ ID NO: 669), UGGgugagga (SEQ ID NO: 2872), AAUguaggua (SEQ ID NO: 4505), UAGguuagca (SEQ ID NO: 4506), GGGguaggua (SEQ ID NO: 2258), GAGguauugc (SEQ ID NO: 4507), AUUguacaca (SEQ ID NO: 4508), GAAguaggua (SEQ ID NO: 4509), GGAguaagcu (SEQ ID NO: 2212), UAGguaugug (SEQ ID NO: 2553), GAGgugaaua (SEQ ID NO: 2007), GAGgugggau (SEQ ID NO: 2056), AAGguaaucu (SEQ ID NO: 163), GGUgugaguu (SEQ ID NO: 4510), AACgugaguu (SEQ ID NO: 4511), GAGguaaccg (SEQ ID NO: 4512), UAGguaagga (SEQ ID NO: 2488), AUUguaagaa (SEQ ID NO: 4513), UGGgugagca (SEQ ID NO: 2870), AAGguaaggc (SEQ ID NO: 150), CCAguaucgu (SEQ ID NO: 4514), CCGgugggug (SEQ ID NO: 4515), GAGguagugu (SEQ ID NO: 4516), ACGgugggaa (SEQ ID NO: 4517), GAGgugaccu (SEQ ID NO: 2011), CACguaugua (SEQ ID NO: 4518), AGGgugggga (SEQ ID NO: 799), AAUguaaguc (SEQ ID NO: 490), AAAguuaagu (SEQ ID NO: 70), CAUgugagug (SEQ ID NO: 1541), AGAguauguc (SEQ ID NO: 694), GCGguaugac (SEQ ID NO: 4519), CGGgugaguu (SEQ ID NO: 1643), CCGguauuuu (SEQ ID NO: 4520), GAGguagaac (SEQ ID NO: 4521), UAGguaugaa (SEQ ID NO: 2545), CAGgcgcgug (SEQ ID NO: 4522), CAAguaaguc (SEQ ID NO: 1027), AGUguaagau (SEQ ID NO: 816), AAGguucuac (SEQ ID NO: 4523), CCAguaagua (SEQ ID NO: 1555), GAGguagcag (SEQ ID NO: 4524), CAGgucuguu (SEQ ID NO: 1312), CAGguacaau (SEQ ID NO: 1162), CCGguaaaga (SEQ ID NO: 1574), UAAgugcugu (SEQ ID NO: 4525), AGGgugagaa (SEQ ID NO: 786), CUCguaaggu (SEQ ID NO: 4526), CAGgucagcu (SEQ ID NO: 4527), CAGguaaggc (SEQ ID NO: 1144), AGGgugcagg (SEQ ID NO: 4528), GAGgugaaac (SEQ ID NO: 4529), AGGguaagua (SEQ ID NO: 740), AAUguaugcc (SEQ ID NO: 4530), AAGguaagca (SEQ ID NO: 145), ACGguacggu (SEQ ID NO: 587), AAGguaauga (SEQ ID NO: 164), UCUgcucaau (SEQ ID NO: 4531), ACGguaaugu (SEQ ID NO: 4532), AAGguaguug (SEQ ID NO: 4533), ACGguaagug (SEQ ID NO: 580), CAGgugauga (SEQ ID NO: 4534), GAGguaacac (SEQ ID NO: 4535), GAGguaggua (SEQ ID NO: 1937), CAGguaccuu (SEQ ID NO: 1179), CAGguaauaa (SEQ ID NO: 1150), UUGgugggug (SEQ ID NO: 3016), CUGguaauga (SEQ ID NO: 1710), UAGguaaguc (SEQ ID NO: 2492), AGGgugugac (SEQ ID NO: 4536), GAGgcaauaa (SEQ ID NO: 4537), GUGguaaagc (SEQ ID NO: 4538), CUGgugggcg (SEQ ID NO: 4539), GAUguauguu (SEQ ID NO: 2128), AGGgugagac (SEQ ID NO: 787), UCGgucagca (SEQ ID NO: 4540), AUGgugauua (SEQ ID NO: 4541), CGAgugugua (SEQ ID NO: 4542), CAGguuggug (SEQ ID NO: 1488), AGCgcaagua (SEQ ID NO: 4543), UGGguacguu (SEQ ID NO: 4544), GAGguauuug (SEQ ID NO: 1974), AGUguacaua (SEQ ID NO: 4545), AUGguaagua (SEQ ID NO: 898), ACAguagguu (SEQ ID NO: 4546), AAGgugagag (SEQ ID NO: 337), UUGgugaagu (SEQ ID NO: 4547), AAAguaugua (SEQ ID NO: 43), UGGguaagga (SEQ ID NO: 4548), UAGgugccuu (SEQ ID NO: 4549), and CCUgugggug (SEQ ID NO: 4550).

Additional exemplary gene sequences and splice site sequences (e.g., 5′ splice site sequences) include UCCguaaguu (SEQ ID NO: 4551), GUGguaaacg (SEQ ID NO: 4552), CGGgugcggu (SEQ ID NO: 4553), CAUguacuuc (SEQ ID NO: 4554), AGAguaaagg (SEQ ID NO: 4555), CGCgugagua (SEQ ID NO: 4556), AGAgugggca (SEQ ID NO: 4557), AGAguaagcc (SEQ ID NO: 4558), AGAguaaaca (SEQ ID NO: 4559), GUGguuauga (SEQ ID NO: 4560), AGGguaauaa (SEQ ID NO: 4561), UGAguaagac (SEQ ID NO: 4562), AGAguuuguu (SEQ ID NO: 4563), CGGgucugca (SEQ ID NO: 4564), CAGguaaguc (SEQ ID NO: 4565), AAGguagaau (SEQ ID NO: 4566), CAGgucccuc (SEQ ID NO: 4567), AGAguaaugg (SEQ ID NO: 4568), GAGgucuaag (SEQ ID NO: 4569), AGAguagagu (SEQ ID NO: 4570), AUGgucagua (SEQ ID NO: 4571), GAGgccuggg (SEQ ID NO: 4572), AAGguguggc (SEQ ID NO: 4573), AGAgugaucu (SEQ ID NO: 4574), AAGguaucca (SEQ ID NO: 4575), UUCguaagua (SEQ ID NO: 4576), UAAgugggug (SEQ ID NO: 4577), GCCgugaacg (SEQ ID NO: 4578), GAGguugugg (SEQ ID NO: 4579), UAUguaugca (SEQ ID NO: 4580), UGUguaacaa (SEQ ID NO: 4581), AGGguauuag (SEQ ID NO: 4582), UGAguauauc (SEQ ID NO: 4583), AGAguuugug (SEQ ID NO: 4584), GAGgucgcug (SEQ ID NO: 4585), GAGgucaucg (SEQ ID NO: 4586), ACGguaaagc (SEQ ID NO: 4587), UGAguacuug (SEQ ID NO: 4588), CGAgucgccg (SEQ ID NO: 4589), CUGguacguc (SEQ ID NO: 4590), AGGguauugc (SEQ ID NO: 4591), GAAgugaaug (SEQ ID NO: 4592), CAGaugaguc (SEQ ID NO: 4593), UGGguauugg (SEQ ID NO: 4594), UGAguaaaga (SEQ ID NO: 4595), GUGguuccug (SEQ ID NO: 4596), UGAgcaagua (SEQ ID NO: 4597), UAUguaagag (SEQ ID NO: 4598), AAGgucuugc (SEQ ID NO: 4599), AAAgcaugug (SEQ ID NO: 4600), AGAguacagu (SEQ ID NO: 4601), GUGguaaucc (SEQ ID NO: 4602), CAGguagagg (SEQ ID NO: 4603), AAGguacaac (SEQ ID NO: 4604), UGGgcagcau (SEQ ID NO: 4605), CCGgucauca (SEQ ID NO: 4606), CCGguuugua (SEQ ID NO: 4607), UGAguaaggg (SEQ ID NO: 4608), GAAguaugua (SEQ ID NO: 4609), GGGguagcuc (SEQ ID NO: 4610), GCUguacaua (SEQ ID NO: 4611), CUGgucucuu (SEQ ID NO: 4612), GUGguaaaug (SEQ ID NO: 4613), AUCguaagug (SEQ ID NO: 4614), GAGgcaugua (SEQ ID NO: 4615), AAGgucuccc (SEQ ID NO: 4616), UGGgugcguu (SEQ ID NO: 4617), UGUguagguu (SEQ ID NO: 4618), GAAgugagca (SEQ ID NO: 4619), GGUguaauuu (SEQ ID NO: 4620), CUGgugaaau (SEQ ID NO: 4621), AUCguaaguc (SEQ ID NO: 4622), AGAguaaucc (SEQ ID NO: 4623), GGAguagguc (SEQ ID NO: 4624), GAGguaccaa (SEQ ID NO: 4625), CUUguaggug (SEQ ID NO: 4626), AAGguauaag (SEQ ID NO: 4627), AGAguuggua (SEQ ID NO: 4628), AUGguuugug (SEQ ID NO: 4629), UGGgucagau (SEQ ID NO: 4630), AGAguaggac (SEQ ID NO: 4631), AGAguagugu (SEQ ID NO: 4632), AGAguaggag (SEQ ID NO: 4633), CAGgucucua (SEQ ID NO: 4634), AAGguggaug (SEQ ID NO: 4635), UGGguaucaa (SEQ ID NO: 4636), GAUguaugga (SEQ ID NO: 4637), AAGguguuuc (SEQ ID NO: 4638), GCAguguaaa (SEQ ID NO: 4639), UUAguaugua (SEQ ID NO: 4640), UCUguaugca (SEQ ID NO: 4641), AAUguaaaau (SEQ ID NO: 4642), AGAguaaauu (SEQ ID NO: 4643), GGGguacuuu (SEQ ID NO: 4644), GAAguuugau (SEQ ID NO: 4645), AAAguagauu (SEQ ID NO: 4646), UGUguagagu (SEQ ID NO: 4647), UGGguaagcg (SEQ ID NO: 4648), CGGguucagg (SEQ ID NO: 4649), AGGguacgac (SEQ ID NO: 4650), UCGguaagaa (SEQ ID NO: 4651), AGGguuggca (SEQ ID NO: 4652), AAAguacagu (SEQ ID NO: 4653), UAAguuaagg (SEQ ID NO: 4654), AUGguaaugu (SEQ ID NO: 4655), GUGguuuuac (SEQ ID NO: 4656), AGAguaacaa (SEQ ID NO: 4657), AAGguagccc (SEQ ID NO: 4658), GCGgugaggc (SEQ ID NO: 4659), AUGguucagc (SEQ ID NO: 4660), AAGguacuua (SEQ ID NO: 4661), AAGguccgug (SEQ ID NO: 4662), UAGguaagcg (SEQ ID NO: 4663), AUGguaccuu (SEQ ID NO: 4664), GCCguggugg (SEQ ID NO: 4665), CUGgugeguc (SEQ ID NO: 4666), CAGguggaaa (SEQ ID NO: 4667), AAAgucugua (SEQ ID NO: 4668), GAGguaaccc (SEQ ID NO: 4669), AGAguauggg (SEQ ID NO: 4670), UAUgccccug (SEQ ID NO: 4671), AAGgugccag (SEQ ID NO: 4672), ACGgugcggc (SEQ ID NO: 4673), AGGguacuga (SEQ ID NO: 4674), AGAguaagcg (SEQ ID NO: 4675), CUGgcaaggg (SEQ ID NO: 4676), CCAgugugug (SEQ ID NO: 4677), GAGguagacg (SEQ ID NO: 4678), CGGgugcggg (SEQ ID NO: 4679), GAUguaagcu (SEQ ID NO: 4680), AUUguauuua (SEQ ID NO: 4681), UGCgugagug (SEQ ID NO: 4682), CUGgucuaua (SEQ ID NO: 4683), GAGgugcuag (SEQ ID NO: 4684), GAGgugccau (SEQ ID NO: 4685), CAGguacguc (SEQ ID NO: 4686), GAGguucagc (SEQ ID NO: 4687), AACguaagaa (SEQ ID NO: 4688), AGAguaguac (SEQ ID NO: 4689), AAGguaacgg (SEQ ID NO: 4690), UAGgugugac (SEQ ID NO: 4691), CCGguaauag (SEQ ID NO: 4692), CAGguaccag (SEQ ID NO: 4693), UUUguaauug (SEQ ID NO: 4694), AAUguacgaa (SEQ ID NO: 4695), CAGguaauga (SEQ ID NO: 4696), AUCgucaagg (SEQ ID NO: 4697), CUGguagaug (SEQ ID NO: 4698), GGGgugcagu (SEQ ID NO: 4699), AGUgugagaa (SEQ ID NO: 4700), GGGguuuuau (SEQ ID NO: 4701), CCUguccccu (SEQ ID NO: 4702), AUUgugaagu (SEQ ID NO: 4703), AAGguaaacg (SEQ ID NO: 4704), UACgucgugg (SEQ ID NO: 4705), AAGgugccau (SEQ ID NO: 4706), GGGgucccag (SEQ ID NO: 4707), UAUguauggu (SEQ ID NO: 4708), CGGguaauua (SEQ ID NO: 4709), CGGguacucc (SEQ ID NO: 4710), CAGgugacuu (SEQ ID NO: 4711), AGUguggguu (SEQ ID NO: 4712), AGAguauggc (SEQ ID NO: 4713), AAGgccaaca (SEQ ID NO: 4714), AAAgcaagua (SEQ ID NO: 4715), UCAguagguc (SEQ ID NO: 4716), GUGguggcgg (SEQ ID NO: 4717), CAUguauccu (SEQ ID NO: 4718), UCGgugagcc (SEQ ID NO: 4719), AUAguugggu (SEQ ID NO: 4720), AAUguuagcu (SEQ ID NO: 4721), AUGgugaaug (SEQ ID NO: 4722), CGGguaaugu (SEQ ID NO: 4723), UCUguaggug (SEQ ID NO: 4724), CCGgugaggc (SEQ ID NO: 4725), UGAguccacu (SEQ ID NO: 4726), CUAguaagag (SEQ ID NO: 4727), CGGguggggc (SEQ ID NO: 4728), CGAguaagca (SEQ ID NO: 4729), UGUgccaauu (SEQ ID NO: 4730), UCGguaagcc (SEQ ID NO: 4731), UAUguaggug (SEQ ID NO: 4732), UUGgugggcc (SEQ ID NO: 4733), GAGgcugggc (SEQ ID NO: 4734), AGAguaacuu (SEQ ID NO: 4735), ACGguagguc (SEQ ID NO: 4736), CAGgcccaga (SEQ ID NO: 4737), CCGguggguu (SEQ ID NO: 4738), AAGgugacgg (SEQ ID NO: 4739), GGGguacagc (SEQ ID NO: 4740), CAUguaaguc (SEQ ID NO: 4741), AUUgugagaa (SEQ ID NO: 4742), UGUguaagga (SEQ ID NO: 4743), UUUguaagau (SEQ ID NO: 4744), AGGgucauuu (SEQ ID NO: 4745), UGGguuuguu (SEQ ID NO: 4746), CGAguaagcc (SEQ ID NO: 4747), GUGgugugua (SEQ ID NO: 4748), AUGguauaac (SEQ ID NO: 4749), UGGguacgua (SEQ ID NO: 4750), AAAguagagu (SEQ ID NO: 4751), UCGguaacug (SEQ ID NO: 4752), AGAguaauga (SEQ ID NO: 4753), AUGguggguc (SEQ ID NO: 4754), AGAguaauau (SEQ ID NO: 4755), CAGguacugg (SEQ ID NO: 4756), UAAgucaguu (SEQ ID NO: 4757), GCGguagaga (SEQ ID NO: 4758), AAGgugaugg (SEQ ID NO: 4759), ACAguauguu (SEQ ID NO: 4760), GAUguacguc (SEQ ID NO: 4761), UAGguuucuc (SEQ ID NO: 4762), GAGgcauggg (SEQ ID NO: 4763), AUAgcuaagu (SEQ ID NO: 4764), GUAgucugua (SEQ ID NO: 4765), AAGgugaacg (SEQ ID NO: 4766), GUGguggucg (SEQ ID NO: 4767), GAGguugauc (SEQ ID NO: 4768), UGAguggguu (SEQ ID NO: 4769), ACUguacgug (SEQ ID NO: 4770), CUGgugacug (SEQ ID NO: 4771), CAAguuaagc (SEQ ID NO: 4772), GAGguaccca (SEQ ID NO: 4773), AACguaacuu (SEQ ID NO: 4774), CAGguuacua (SEQ ID NO: 4775), AGAguuaguc (SEQ ID NO: 4776), UGGgcacguc (SEQ ID NO: 4777), AGUguauggu (SEQ ID NO: 4778), AAGguugcaa (SEQ ID NO: 4779), CAGguuguua (SEQ ID NO: 4780), AAGgcauccc (SEQ ID NO: 4781), GAUguaaggc (SEQ ID NO: 4782), AGGguacggg (SEQ ID NO: 4783), GAGgucaaag (SEQ ID NO: 4784), CAAgugagcg (SEQ ID NO: 4785), AGAguaaucu (SEQ ID NO: 4786), UCGguagcug (SEQ ID NO: 4787), AAAguaguag (SEQ ID NO: 4788), CAGguucguc (SEQ ID NO: 4789), CGUguaugaa (SEQ ID NO: 4790), AGUguaaaaa (SEQ ID NO: 4791), AAGgucucac (SEQ ID NO: 4792), UAGguggagc (SEQ ID NO: 4793), UGAguaggug (SEQ ID NO: 4794), AGAguaugcc (SEQ ID NO: 4795), GAGguugcau (SEQ ID NO: 4796), CAAguaagag (SEQ ID NO: 4797), UCUgugugcc (SEQ ID NO: 4798), GAGgugaugc (SEQ ID NO: 4799), GGGgugauaa (SEQ ID NO: 4800), CCCgugagcc (SEQ ID NO: 4801), AGAguaacug (SEQ ID NO: 4802), GCGguaagua (SEQ ID NO: 4803), AGAguacauc (SEQ ID NO: 4804), UCGgucuggg (SEQ ID NO: 4805), UAAguaucuc (SEQ ID NO: 4806), GGCguagguu (SEQ ID NO: 4807), AGAguacgcc (SEQ ID NO: 4808), GAUgucuucu (SEQ ID NO: 4809), AGGgcaaggu (SEQ ID NO: 4810), CGAguaugau (SEQ ID NO: 4811), AUGguagagu (SEQ ID NO: 4812), CAAguacgag (SEQ ID NO: 4813), UCGguaugau (SEQ ID NO: 4814), CCGguguguu (SEQ ID NO: 4815), AGGgucugug (SEQ ID NO: 4816), GGAguaggcu (SEQ ID NO: 4817), AAGgucuaug (SEQ ID NO: 4818), GCAgugcgug (SEQ ID NO: 4819), UGGgugagaa (SEQ ID NO: 4820), AGGguaaagu (SEQ ID NO: 4821), GAGguaggac (SEQ ID NO: 4822), CUAguaagca (SEQ ID NO: 4823), UUAguaggcu (SEQ ID NO: 4824), CUGgugggau (SEQ ID NO: 4825), CUGguuagua (SEQ ID NO: 4826), AAGguacgug (SEQ ID NO: 4827), CGGgugagau (SEQ ID NO: 4828), AAGgugcaug (SEQ ID NO: 4829), AAUgugggcu (SEQ ID NO: 4830), CAGguugacu (SEQ ID NO: 4831), CAGguuacag (SEQ ID NO: 4832), GCGguaacau (SEQ ID NO: 4833), AUUgucaguc (SEQ ID NO: 4834), CAAguauaca (SEQ ID NO: 4835), GAUgucegcc (SEQ ID NO: 4836), AAGgugcgga (SEQ ID NO: 4837), AACguaagag (SEQ ID NO: 4838), UGGguuggua (SEQ ID NO: 4839), CAAguguaag (SEQ ID NO: 4840), GUGguaacgu (SEQ ID NO: 4841), CUGgugauca (SEQ ID NO: 4842), AGGguggggc (SEQ ID NO: 4843), UCGguaaaga (SEQ ID NO: 4844), CAGguacacc (SEQ ID NO: 4845), CGGguaaggg (SEQ ID NO: 4846), CAAguuugcu (SEQ ID NO: 4847), ACAgugcgug (SEQ ID NO: 4848), UUGguauggg (SEQ ID NO: 4849), GAGgcucauc (SEQ ID NO: 4850), CUGguaauag (SEQ ID NO: 4851), AUGguggaua (SEQ ID NO: 4852), UCAgugaauu (SEQ ID NO: 4853), AAUguaauua (SEQ ID NO: 4854), GCAgucuaaa (SEQ ID NO: 4855), AAGguauucu (SEQ ID NO: 4856), GAGgucauca (SEQ ID NO: 4857), UGGguccaug (SEQ ID NO: 4858), AGAguuugua (SEQ ID NO: 4859), AGGguagacu (SEQ ID NO: 4860), AAGguaggac (SEQ ID NO: 4861), UGUguguuga (SEQ ID NO: 4862), UCAguacgug (SEQ ID NO: 4863), AUGgucucuc (SEQ ID NO: 4864), UGAguuagua (SEQ ID NO: 4865), UGAguaaagu (SEQ ID NO: 4866), GAGgugaccg (SEQ ID NO: 4867), GAGguauauc (SEQ ID NO: 4868), CAGgugccau (SEQ ID NO: 4869), AGAgugguga (SEQ ID NO: 4870), GUUguaagaa (SEQ ID NO: 4871), AGAguaaaua (SEQ ID NO: 4872), AGGgugaagg (SEQ ID NO: 4873), CUGguagauu (SEQ ID NO: 4874), GAGguucagg (SEQ ID NO: 4875), AGGgucuuca (SEQ ID NO: 4876), CUGguaaccu (SEQ ID NO: 4877), ACAguacuga (SEQ ID NO: 4878), AGAguggguc (SEQ ID NO: 4879), AUGguaugag (SEQ ID NO: 4880), AAGguuauau (SEQ ID NO: 4881), AGAguauagu (SEQ ID NO: 4882), AAAguaugaa (SEQ ID NO: 4883), UAGguggcua (SEQ ID NO: 4884), ACCguauggg (SEQ ID NO: 4885), AAAguauaau (SEQ ID NO: 4886), UUUguauggc (SEQ ID NO: 4887), GGGgucgcgu (SEQ ID NO: 4888), GUGgugguuu (SEQ ID NO: 4889), CAGguuugac (SEQ ID NO: 4890), GGAguaggcg (SEQ ID NO: 4891), GAGguacccu (SEQ ID NO: 4892), AUGgugugca (SEQ ID NO: 4893), GUGguuggug (SEQ ID NO: 4894), AAAguaugcu (SEQ ID NO: 4895), UAAguuacau (SEQ ID NO: 4896), ACAguaugag (SEQ ID NO: 4897), GGAguauguu (SEQ ID NO: 4898), UUUgugagaa (SEQ ID NO: 4899), AAUgugcguu (SEQ ID NO: 4900), CAGguagagu (SEQ ID NO: 4901), AUGguguuaa (SEQ ID NO: 4902), CAUgugeguc (SEQ ID NO: 4903), AUAguuggau (SEQ ID NO: 4904), GAGguacgua (SEQ ID NO: 4905), GUUgugagaa (SEQ ID NO: 4906), CAAguacauc (SEQ ID NO: 4907), GAGguaguuu (SEQ ID NO: 4908), ACUguacaga (SEQ ID NO: 4909), CCGguuguga (SEQ ID NO: 4910), UGGgucagug (SEQ ID NO: 4911), GUAguaagaa (SEQ ID NO: 4912), GACguacuuu (SEQ ID NO: 4913), AGAgucaguc (SEQ ID NO: 4914), UAGguuaguu (SEQ ID NO: 4915), AGGgcagcag (SEQ ID NO: 4916), AAGguccuac (SEQ ID NO: 4917), AAUguaauug (SEQ ID NO: 4918), CAGgugcggg (SEQ ID NO: 4919), CUGguaaugg (SEQ ID NO: 4920), CAAguagccc (SEQ ID NO: 4921), GAAgucaguu (SEQ ID NO: 4922), ACAguaauug (SEQ ID NO: 4923), UUAguuagua (SEQ ID NO: 4924), CCUguauuuu (SEQ ID NO: 4925), AUCguaagaa (SEQ ID NO: 4926), CCAgugagca (SEQ ID NO: 4927), GAAguaaggc (SEQ ID NO: 4928), UGAgugggua (SEQ ID NO: 4929), UCAgugguag (SEQ ID NO: 4930), UCUguacagg (SEQ ID NO: 4931), CGAgugagug (SEQ ID NO: 4932), UCCguaugug (SEQ ID NO: 4933), CAUgccguuu (SEQ ID NO: 4934), AAAgugacuu (SEQ ID NO: 4935), AGAguaggca (SEQ ID NO: 4936), GAAguaagag (SEQ ID NO: 4937), CAGgcagguu (SEQ ID NO: 4938), UUGguagagc (SEQ ID NO: 4939), AAGguggaaa (SEQ ID NO: 4940), GAGgcagguc (SEQ ID NO: 4941), AUGguacgac (SEQ ID NO: 4942), AGGguaggaa (SEQ ID NO: 4943), AGGguaggua (SEQ ID NO: 4944), UUGguaaggu (SEQ ID NO: 4945), AUGguacaga (SEQ ID NO: 4946), CAGguagagc (SEQ ID NO: 4947), UAGguaaggu (SEQ ID NO: 4948), GGGguuagag (SEQ ID NO: 4949), AAGguaucaa (SEQ ID NO: 4950), GAGguagccc (SEQ ID NO: 4951), CAGgugccuc (SEQ ID NO: 4952), GCAguaagag (SEQ ID NO: 4953), ACGguagagu (SEQ ID NO: 4954), UGGguaaugg (SEQ ID NO: 4955), CUGgucaguu (SEQ ID NO: 4956), GUGguacauu (SEQ ID NO: 4957), AAAguagguu (SEQ ID NO: 4958), AAGgccaaga (SEQ ID NO: 4959), CGGgugggca (SEQ ID NO: 4960), ACGguccggg (SEQ ID NO: 4961), CGAguaugag (SEQ ID NO: 4962), CUGguaugcc (SEQ ID NO: 4963), GAGguggaug (SEQ ID NO: 4964), CAGgccuuuc (SEQ ID NO: 4965), AAAguacauc (SEQ ID NO: 4966), AAAguaauca (SEQ ID NO: 4967), GAGguaacug (SEQ ID NO: 4968), CUGguaaaga (SEQ ID NO: 4969), CGUguaagca (SEQ ID NO: 4970), UGGgcaagua (SEQ ID NO: 4971), GCGguggcga (SEQ ID NO: 4972), GAGguggccg (SEQ ID NO: 4973), AUUgcaugca (SEQ ID NO: 4974), ACGgugacug (SEQ ID NO: 4975), CAGgucagau (SEQ ID NO: 4976), AGAguaacuc (SEQ ID NO: 4977), UGAguaacag (SEQ ID NO: 4978), AAGguacccg (SEQ ID NO: 4979), AGGguaggcu (SEQ ID NO: 4980), GGGgcaggac (SEQ ID NO: 4981), CCUguaagug (SEQ ID NO: 4982), AUUguaagug (SEQ ID NO: 4983), ACUguacgag (SEQ ID NO: 4984), GUAguagugu (SEQ ID NO: 4985), AGAguaugag (SEQ ID NO: 4986), UCAguguggg (SEQ ID NO: 4987), UGGguauaua (SEQ ID NO: 4988), UAGguagcua (SEQ ID NO: 4989), GGGguaaaga (SEQ ID NO: 4990), AGGguuacuu (SEQ ID NO: 4991), CAUguaaaug (SEQ ID NO: 4992), GGAguaguaa (SEQ ID NO: 4993), CAGgucaauc (SEQ ID NO: 4994), CGGguuagug (SEQ ID NO: 4995), UAGguacaug (SEQ ID NO: 4996), UAGguuaaga (SEQ ID NO: 4997), UGGguaccuu (SEQ ID NO: 4998), CGGguggaca (SEQ ID NO: 4999), CAGgucuuac (SEQ ID NO: 5000), AAGguggagc (SEQ ID NO: 5001), AUGguaacca (SEQ ID NO: 5002), UCGguaaguu (SEQ ID NO: 5003), UAUguacaaa (SEQ ID NO: 5004), AAUguagauu (SEQ ID NO: 5005), GUAgcuagua (SEQ ID NO: 5006), AAGguauugg (SEQ ID NO: 5007), GAGgucuuug (SEQ ID NO: 5008), GAAguucagg (SEQ ID NO: 5009), UGGguaucac (SEQ ID NO: 5010), AGAguacugg (SEQ ID NO: 5011), CAGguuaaug (SEQ ID NO: 5012), AGGguacgug (SEQ ID NO: 5013), AGGgcacagg (SEQ ID NO: 5014), CUGguuaguu (SEQ ID NO: 5015), UUGguacgag (SEQ ID NO: 5016), ACGgugauca (SEQ ID NO: 5017), CCUgugagag (SEQ ID NO: 5018), GAGgugaagu (SEQ ID NO: 5019), AAGguacauc (SEQ ID NO: 5020), UCUguaugug (SEQ ID NO: 5021), UUGguggaag (SEQ ID NO: 5022), UGGgcagguu (SEQ ID NO: 5023), GAAguggagc (SEQ ID NO: 5024), ACAguaagac (SEQ ID NO: 5025), CGGguaccaa (SEQ ID NO: 5026), CAAguacguc (SEQ ID NO: 5027), AGAgugaggg (SEQ ID NO: 5028), CGGguaagaa (SEQ ID NO: 5029), AAUguaggug (SEQ ID NO: 5030), AUCgugugcu (SEQ ID NO: 5031), UAGgucaugg (SEQ ID NO: 5032), CAGguuuuga (SEQ ID NO: 5033), AAGgcaugca (SEQ ID NO: 5034), GAGgugcugc (SEQ ID NO: 5035), AAGguuaaua (SEQ ID NO: 5036), CAGguucauc (SEQ ID NO: 5037), GCGguaggug (SEQ ID NO: 5038), GACgugagua (SEQ ID NO: 5039), CAGgucuacu (SEQ ID NO: 5040), UUGguaugag (SEQ ID NO: 5041), AGCgugggca (SEQ ID NO: 5042), AUGguaaggu (SEQ ID NO: 5043), AUGguaccuc (SEQ ID NO: 5044), UUGguauggu (SEQ ID NO: 5045), UAUguaugaa (SEQ ID NO: 5046), UGGguauggg (SEQ ID NO: 5047), GAUguaaaua (SEQ ID NO: 5048), CCGguaaguu (SEQ ID NO: 5049), GAGgucugaa (SEQ ID NO: 5050), GAGgugcgag (SEQ ID NO: 5051), CUGgucagcc (SEQ ID NO: 5052), CAGguuuugu (SEQ ID NO: 5053), CGGguggugu (SEQ ID NO: 5054), UAAguuagua (SEQ ID NO: 5055), UUUgugugug (SEQ ID NO: 5056), CAGguuaacc (SEQ ID NO: 5057), UUGguacuuu (SEQ ID NO: 5058), GCUguaaggc (SEQ ID NO: 5059), AGGguggcug (SEQ ID NO: 5060), GAUguaaaaa (SEQ ID NO: 5061), AAGgucaaaa (SEQ ID NO: 5062), CAGguagcgc (SEQ ID NO: 5063), CAGguuuggc (SEQ ID NO: 5064), GAGgugguuu (SEQ ID NO: 5065), CGGguaaaua (SEQ ID NO: 5066), CUGguucggu (SEQ ID NO: 5067), GGAgugagcc (SEQ ID NO: 5068), AAGgugcgcg (SEQ ID NO: 5069), GAAguacauc (SEQ ID NO: 5070), AGUgucugua (SEQ ID NO: 5071), CCCgugagcu (SEQ ID NO: 5072), GAGguucaca (SEQ ID NO: 5073), CUAgugggua (SEQ ID NO: 5074), GAGguaacua (SEQ ID NO: 5075), UCGguauguc (SEQ ID NO: 5076), UAAguauuug (SEQ ID NO: 5077), CAGguaagcg (SEQ ID NO: 5078), GAGgugguaa (SEQ ID NO: 5079), CGAguaagag (SEQ ID NO: 5080), CCGguaagcu (SEQ ID NO: 5081), GAGgucuugu (SEQ ID NO: 5082), AAGguggguc (SEQ ID NO: 5083), CACguaagug (SEQ ID NO: 5084), AGUguaauga (SEQ ID NO: 5085), AAAgugugua (SEQ ID NO: 5086), GGAgugccaa (SEQ ID NO: 5087), CACgugaguu (SEQ ID NO: 5088), AAGguuggau (SEQ ID NO: 5089), UAUguaaaua (SEQ ID NO: 5090), CUGguaggaa (SEQ ID NO: 5091), UAUguaaacu (SEQ ID NO: 5092), AAUguauuuu (SEQ ID NO: 5093), CUGgcaagug (SEQ ID NO: 5094), UGUgugguau (SEQ ID NO: 5095), UAUguauguu (SEQ ID NO: 5096), UUGgugacuc (SEQ ID NO: 5097), GGAguaaggu (SEQ ID NO: 5098), AAGguagaug (SEQ ID NO: 5099), UGGguagggu (SEQ ID NO: 5100), AAUguaauuc (SEQ ID NO: 5101), GUGguauggc (SEQ ID NO: 5102), GGAguggguu (SEQ ID NO: 5103), AGGguaccac (SEQ ID NO: 5104), UAGgugacag (SEQ ID NO: 5105), ACAguaggca (SEQ ID NO: 5106), AUGguuugaa (SEQ ID NO: 5107), GCAguaacua (SEQ ID NO: 5108), CCGguaggua (SEQ ID NO: 5109), AGAguaggcc (SEQ ID NO: 5110), AAGguugaca (SEQ ID NO: 5111), CUGgugugua (SEQ ID NO: 5112), GAAgucuguc (SEQ ID NO: 5113), UGGgcucgga (SEQ ID NO: 5114), CAGguagccu (SEQ ID NO: 5115), AGAguaggua (SEQ ID NO: 5116), UAAguauguc (SEQ ID NO: 5117), CUGguauauc (SEQ ID NO: 5118), GAGguguguu (SEQ ID NO: 5119), AUGgugcaug (SEQ ID NO: 5120), AAGguacgcc (SEQ ID NO: 5121), UGAguaacua (SEQ ID NO: 5122), GAGgugacag (SEQ ID NO: 5123), GUUguccugu (SEQ ID NO: 5124), UUGgugucuu (SEQ ID NO: 5125), AAUgugaagg (SEQ ID NO: 5126), UUGguggaua (SEQ ID NO: 5127), UAGguguguu (SEQ ID NO: 5128), CUGgcaaguu (SEQ ID NO: 5129), GCAguaagau (SEQ ID NO: 5130), GCGguggaaa (SEQ ID NO: 5131), UGCguccagc (SEQ ID NO: 5132), AAAguggagu (SEQ ID NO: 5133), CGUgugagcc (SEQ ID NO: 5134), AGAguacugu (SEQ ID NO: 5135), CAGguauagc (SEQ ID NO: 5136), UACguaagga (SEQ ID NO: 5137), AAGgucuuua (SEQ ID NO: 5138), AAGguggucu (SEQ ID NO: 5139), GGGguaaauu (SEQ ID NO: 5140), UCAgugagga (SEQ ID NO: 5141), AGAguacguu (SEQ ID NO: 5142), GAGgucguca (SEQ ID NO: 5143), UAGguuugau (SEQ ID NO: 5144), CAUguaaacc (SEQ ID NO: 5145), AAGguggcac (SEQ ID NO: 5146), CAGguagaug (SEQ ID NO: 5147), AACguaaaag (SEQ ID NO: 5148), UAGgucucug (SEQ ID NO: 5149), AUAguaggug (SEQ ID NO: 5150), UAGgcaagag (SEQ ID NO: 5151), UAGgcacggc (SEQ ID NO: 5152), AAGgucuuca (SEQ ID NO: 5153), CCAguaugcu (SEQ ID NO: 5154), CAAgugaguu (SEQ ID NO: 5155), CAGgucucaa (SEQ ID NO: 5156), CAGguuacau (SEQ ID NO: 5157), GGAgugagca (SEQ ID NO: 5158), AGAguacgca (SEQ ID NO: 5159), CUGguguugg (SEQ ID NO: 5160), AAGguacuca (SEQ ID NO: 5161), CUAguaaggg (SEQ ID NO: 5162), AGAguaaaag (SEQ ID NO: 5163), AAGguaacga (SEQ ID NO: 5164), CUGguccccg (SEQ ID NO: 5165), UAAguauggg (SEQ ID NO: 5166), GAGgucgagc (SEQ ID NO: 5167), UUGguauaua (SEQ ID NO: 5168), AAAgucaagg (SEQ ID NO: 5169), AAGgucuagg (SEQ ID NO: 5170), CGAguagguc (SEQ ID NO: 5171), AGGguucguu (SEQ ID NO: 5172), GAGgcaggcc (SEQ ID NO: 5173), CUAguauuac (SEQ ID NO: 5174), ACGguaugug (SEQ ID NO: 5175), UAGgugguuc (SEQ ID NO: 5176), AGAguauaac (SEQ ID NO: 5177), UUGgugcguc (SEQ ID NO: 5178), ACCguuaucu (SEQ ID NO: 5179), CCAgugauga (SEQ ID NO: 5180), GAAguaugca (SEQ ID NO: 5181), GAAguauggc (SEQ ID NO: 5182), CCGguaggac (SEQ ID NO: 5183), AAUguaagca (SEQ ID NO: 5184), AGAguaauug (SEQ ID NO: 5185), AGGguugguu (SEQ ID NO: 5186), GUGguaggag (SEQ ID NO: 5187), AAGgcaguuu (SEQ ID NO: 5188), CAAguaagcc (SEQ ID NO: 5189), CUGgcaagua (SEQ ID NO: 5190), CAGgcaugau (SEQ ID NO: 5191), AGGguaauug (SEQ ID NO: 5192), GGGguaaccu (SEQ ID NO: 5193), AAAguaacua (SEQ ID NO: 5194), UAGgucugcc (SEQ ID NO: 5195), ACGguaugaa (SEQ ID NO: 5196), AGUguauggg (SEQ ID NO: 5197), UGGguuggca (SEQ ID NO: 5198), UAGguaaacu (SEQ ID NO: 5199), AGAgugggua (SEQ ID NO: 5200), AGAguauuug (SEQ ID NO: 5201), AGUguaggaa (SEQ ID NO: 5202), CUUguacgua (SEQ ID NO: 5203), GAUgugagau (SEQ ID NO: 5204), CAGgcagcca (SEQ ID NO: 5205), AAGgucacug (SEQ ID NO: 5206), AAGgucugac (SEQ ID NO: 5207), UAGguuccuu (SEQ ID NO: 5208), CUGgugcuuu (SEQ ID NO: 5209), UGAguuggug (SEQ ID NO: 5210), UUGgugggau (SEQ ID NO: 5211), UGAguagggu (SEQ ID NO: 5212), UCGgugaggu (SEQ ID NO: 5213), AAAguaaaga (SEQ ID NO: 5214), AAGgcaaguc (SEQ ID NO: 5215), CGGguaaagc (SEQ ID NO: 5216), AAAguuaguu (SEQ ID NO: 5217), UUAguaagca (SEQ ID NO: 5218), GAGgucacau (SEQ ID NO: 5219), UAAgugguau (SEQ ID NO: 5220), UAGgugcuuu (SEQ ID NO: 5221), GGAguaggca (SEQ ID NO: 5222), UGAguaagga (SEQ ID NO: 5223), CAGguggagc (SEQ ID NO: 5224), GAUguagaag (SEQ ID NO: 5225), AAUgccugcc (SEQ ID NO: 5226), AUGguaaggc (SEQ ID NO: 5227), UGGguaauau (SEQ ID NO: 5228), CUGguaccuc (SEQ ID NO: 5229), CACgugagcc (SEQ ID NO: 5230), UGAguuugug (SEQ ID NO: 5231), CCGguagugu (SEQ ID NO: 5232), AAAgugacaa (SEQ ID NO: 5233), GAAguggguu (SEQ ID NO: 5234), CAGgugcagc (SEQ ID NO: 5235), GAGgugggcc (SEQ ID NO: 5236), UAUgugcguc (SEQ ID NO: 5237), GGGguacugg (SEQ ID NO: 5238), CUGguagguu (SEQ ID NO: 5239), UUGgcauguu (SEQ ID NO: 5240), AAUguaauac (SEQ ID NO: 5241), UAGgccggug (SEQ ID NO: 5242), AGAgucagua (SEQ ID NO: 5243), UAAguaaauc (SEQ ID NO: 5244), CAGguuccuc (SEQ ID NO: 5245), UAGguacgau (SEQ ID NO: 5246), AGAguuagug (SEQ ID NO: 5247), GCAguaagug (SEQ ID NO: 5248), AGGgugguag (SEQ ID NO: 5249), GGAguaaugu (SEQ ID NO: 5250), GAUguaaguc (SEQ ID NO: 5251), CCAguuucgu (SEQ ID NO: 5252), AAGguucggg (SEQ ID NO: 5253), AUGguggagu (SEQ ID NO: 5254), AAGguaccgg (SEQ ID NO: 5255), GAAgugcgaa (SEQ ID NO: 5256), UGGgucaguu (SEQ ID NO: 5257), AAGguguaga (SEQ ID NO: 5258), UGGguaggcc (SEQ ID NO: 5259), CCAgugaguc (SEQ ID NO: 5260), AAGgucacuu (SEQ ID NO: 5261), AGCgugaggc (SEQ ID NO: 5262), UCCgugguaa (SEQ ID NO: 5263), AGAguacuua (SEQ ID NO: 5264), GGGgucagau (SEQ ID NO: 5265), AAGguggacc (SEQ ID NO: 5266), AGAgugagcg (SEQ ID NO: 5267), AGAgucagau (SEQ ID NO: 5268), UAAguauuac (SEQ ID NO: 5269), AGAguauuuc (SEQ ID NO: 5270), AGAguucagc (SEQ ID NO: 5271), AUGgugaagu (SEQ ID NO: 5272), UAGgugaucc (SEQ ID NO: 5273), GGAguaagau (SEQ ID NO: 5274), UAGguaccaa (SEQ ID NO: 5275), AGAguugguc (SEQ ID NO: 5276), GAAgugagac (SEQ ID NO: 5277), AUCguagguu (SEQ ID NO: 5278), GAGguacgcu (SEQ ID NO: 5279), ACGguaaggg (SEQ ID NO: 5280), CAGgcauguc (SEQ ID NO: 5281), UUAguaagau (SEQ ID NO: 5282), UGAguagguu (SEQ ID NO: 5283), AGGguacgaa (SEQ ID NO: 5284), ACGguauguu (SEQ ID NO: 5285), AGGguacugu (SEQ ID NO: 5286), UUGguaugga (SEQ ID NO: 5287), UAAguaacug (SEQ ID NO: 5288), GCGgucagcc (SEQ ID NO: 5289), UUUgugaguc (SEQ ID NO: 5290), GUGgucagug (SEQ ID NO: 5291), CUGgucugua (SEQ ID NO: 5292), GAGguucuua (SEQ ID NO: 5293), AUGguacuga (SEQ ID NO: 5294), AAUgugcuuu (SEQ ID NO: 5295), AGGguggcgu (SEQ ID NO: 5296), CCGgcaggaa (SEQ ID NO: 5297), CAUguggguc (SEQ ID NO: 5298), UUGguuuguu (SEQ ID NO: 5299), CAGguucugu (SEQ ID NO: 5300), ACGguaagcg (SEQ ID NO: 5301), CUGgucagua (SEQ ID NO: 5302), UCAguaggcu (SEQ ID NO: 5303), UGAguaggac (SEQ ID NO: 5304), CAGguuuuaa (SEQ ID NO: 5305), GAGguguccc (SEQ ID NO: 5306), AGGguggguu (SEQ ID NO: 5307), GUGgugagac (SEQ ID NO: 5308), CACguaggga (SEQ ID NO: 5309), GUGguauuuu (SEQ ID NO: 5310), GAGauauccu (SEQ ID NO: 5311), AAGgugaaca (SEQ ID NO: 5312), UAAguagggc (SEQ ID NO: 5313), CUGgugcggg (SEQ ID NO: 5314), CUGgucaaua (SEQ ID NO: 5315), AGAguaaaaa (SEQ ID NO: 5316), AAGgugcagu (SEQ ID NO: 5317), CGGguaagca (SEQ ID NO: 5318), AAAgugagcc (SEQ ID NO: 5319), AUGguaauca (SEQ ID NO: 5320), GCAguacgug (SEQ ID NO: 5321), AUGguacaug (SEQ ID NO: 5322), AAGguuaaga (SEQ ID NO: 5323), CGGguaaaug (SEQ ID NO: 5324), GAGguucgca (SEQ ID NO: 5325), GAGgcucugg (SEQ ID NO: 5326), AUGgugggac (SEQ ID NO: 5327), AACgugguag (SEQ ID NO: 5328), AAGgugauag (SEQ ID NO: 5329), GGGguuugca (SEQ ID NO: 5330), CAUguaaggg (SEQ ID NO: 5331), UCAguugagu (SEQ ID NO: 5332), AAAgugcggc (SEQ ID NO: 5333), AGAgugagcc (SEQ ID NO: 5334), AUGgcaagaa (SEQ ID NO: 5335), ACAguaaggu (SEQ ID NO: 5336), AAGgucucua (SEQ ID NO: 5337), GUGguaaaaa (SEQ ID NO: 5338), AAAguaggug (SEQ ID NO: 5339), UAGgugcacu (SEQ ID NO: 5340), GUCgugguau (SEQ ID NO: 5341), CAGguauagg (SEQ ID NO: 5342), UGAgugagag (SEQ ID NO: 5343), ACUgugagcc (SEQ ID NO: 5344), AUCguuaguu (SEQ ID NO: 5345), UUUguaccaa (SEQ ID NO: 5346), UGGgugagau (SEQ ID NO: 5347), AGAgugagaa (SEQ ID NO: 5348), AGAguagggg (SEQ ID NO: 5349), AGGgcaagua (SEQ ID NO: 5350), CGGgucagua (SEQ ID NO: 5351), UUGguaugcc (SEQ ID NO: 5352), CGGguuagau (SEQ ID NO: 5353), GGGgugaagu (SEQ ID NO: 5354), CCCgugugaa (SEQ ID NO: 5355), GCAguuugga (SEQ ID NO: 5356), UGCguaagac (SEQ ID NO: 5357), AGAgucugua (SEQ ID NO: 5358), CACgugagca (SEQ ID NO: 5359), AGGguaaaag (SEQ ID NO: 5360), CAGgcugggu (SEQ ID NO: 5361), GAAgucuuca (SEQ ID NO: 5362), AAGgcaaaaa (SEQ ID NO: 5363), GUAguaaaua (SEQ ID NO: 5364), CUAgugagag (SEQ ID NO: 5365), GAAguuucug (SEQ ID NO: 5366), CCUguacgua (SEQ ID NO: 5367), GAGgugcgcg (SEQ ID NO: 5368), AAGguguaaa (SEQ ID NO: 5369), CCAguauguu (SEQ ID NO: 5370), CCGgucagcu (SEQ ID NO: 5371), AUGguuccug (SEQ ID NO: 5372), CAAguuaaau (SEQ ID NO: 5373), AGAguaggcu (SEQ ID NO: 5374), AUGgugggca (SEQ ID NO: 5375), GGAguaagac (SEQ ID NO: 5376), AGGgucacga (SEQ ID NO: 5377), UAGgugauau (SEQ ID NO: 5378), GAAguaaguc (SEQ ID NO: 5379), CGGguaagau (SEQ ID NO: 5380), CAAguagcua (SEQ ID NO: 5381), UGAguaaaau (SEQ ID NO: 5382), GUCguacgug (SEQ ID NO: 5383), AUGguacgua (SEQ ID NO: 5384), CAGgucucgg (SEQ ID NO: 5385), GAGgcauguc (SEQ ID NO: 5386), AGAgugggau (SEQ ID NO: 5387), GUGguuagag (SEQ ID NO: 5388), UGGgugguga (SEQ ID NO: 5389), AAGguuaaac (SEQ ID NO: 5390), CUUguuagcu (SEQ ID NO: 5391), AAAguaggaa (SEQ ID NO: 5392), UAGguuguau (SEQ ID NO: 5393), AGGgugcgcc (SEQ ID NO: 5394), AAGgugggcu (SEQ ID NO: 5395), UAAguaucug (SEQ ID NO: 5396), AAGguaacgu (SEQ ID NO: 5397), AUGguggggc (SEQ ID NO: 5398), CAAguacacg (SEQ ID NO: 5399), GGCguaagug (SEQ ID NO: 5400), AUAguaggac (SEQ ID NO: 5401), AGAgugaggu (SEQ ID NO: 5402), UUUguaaaaa (SEQ ID NO: 5403), GAAguuugua (SEQ ID NO: 5404), CUAguaaucu (SEQ ID NO: 5405), AAGguuuuua (SEQ ID NO: 5406), GAGgugcguu (SEQ ID NO: 5407), UAGgcgagua (SEQ ID NO: 5408), ACCgugagua (SEQ ID NO: 5409), CAGgucccga (SEQ ID NO: 5410), AUGguacugg (SEQ ID NO: 5411), UGAguucagu (SEQ ID NO: 5412), AAUguguggu (SEQ ID NO: 5413), UCCguugguu (SEQ ID NO: 5414), CAGgucagag (SEQ ID NO: 5415), CAGgucccua (SEQ ID NO: 5416), UAGguagacu (SEQ ID NO: 5417), CAAguuaagg (SEQ ID NO: 5418), GAGgugugcg (SEQ ID NO: 5419), GAAgcugccc (SEQ ID NO: 5420), CGAguacgug (SEQ ID NO: 5421), CGGguaggua (SEQ ID NO: 5422), UUGguauuga (SEQ ID NO: 5423), AUUguaugau (SEQ ID NO: 5424), UUGguaugaa (SEQ ID NO: 5425), GAGgugguca (SEQ ID NO: 5426), GCUguaugaa (SEQ ID NO: 5427), CAGguguugc (SEQ ID NO: 5428), CAGguaaaac (SEQ ID NO: 5429), AUAguaaggu (SEQ ID NO: 5430), CUGguuagag (SEQ ID NO: 5431), AGCgugugag (SEQ ID NO: 5432), AAGguuaucu (SEQ ID NO: 5433), CACgugagua (SEQ ID NO: 5434), AGGgucagua (SEQ ID NO: 5435), GAGguauaau (SEQ ID NO: 5436), CAGguuauuu (SEQ ID NO: 5437), AGGguggacu (SEQ ID NO: 5438), AUUguaauuc (SEQ ID NO: 5439), UUUguggguu (SEQ ID NO: 5440), AUGguacgug (SEQ ID NO: 5441), AAGguguucc (SEQ ID NO: 5442), CAGgugacgc (SEQ ID NO: 5443), GAGguacuaa (SEQ ID NO: 5444), ACAguucagu (SEQ ID NO: 5445), GAGgucacgg (SEQ ID NO: 5446), CAAguaaggc (SEQ ID NO: 5447), AAGguuuggg (SEQ ID NO: 5448), AAAgugggcu (SEQ ID NO: 5449), GCGguucuug (SEQ ID NO: 5450), GAGguggagc (SEQ ID NO: 5451), UGAgucagug (SEQ ID NO: 5452), CAGgucaagg (SEQ ID NO: 5453), AGUguaagcu (SEQ ID NO: 5454), GAGgcagaaa (SEQ ID NO: 5455), AAGgucacac (SEQ ID NO: 5456), GAAguagguu (SEQ ID NO: 5457), GUCguaaguu (SEQ ID NO: 5458), AGAguaugca (SEQ ID NO: 5459), CCUgugcaaa (SEQ ID NO: 5460), ACGgugaaaa (SEQ ID NO: 5461), CAGguacgaa (SEQ ID NO: 5462), CAUgugagga (SEQ ID NO: 5463), AGCgugagua (SEQ ID NO: 5464), GGUguguagg (SEQ ID NO: 5465), AACgugagcu (SEQ ID NO: 5466), GAGgugaacu (SEQ ID NO: 5467), AGAguucagu (SEQ ID NO: 5468), AACgugugua (SEQ ID NO: 5469), CAGguugugg (SEQ ID NO: 5470), AAGguacuag (SEQ ID NO: 5471), UCAgugaaaa (SEQ ID NO: 5472), AAUgucuggu (SEQ ID NO: 5473), ACGguaaaau (SEQ ID NO: 5474), CUGguguaag (SEQ ID NO: 5475), GAGgugcgaa (SEQ ID NO: 5476), AGGguuucuc (SEQ ID NO: 5477), CAGguagccc (SEQ ID NO: 5478), AUUguauugg (SEQ ID NO: 5479), AUGguacuua (SEQ ID NO: 5480), GAGgcccgac (SEQ ID NO: 5481), UCGguaagac (SEQ ID NO: 5482), CGGgcuguag (SEQ ID NO: 5483), UAUgugugug (SEQ ID NO: 5484), UAGguagaaa (SEQ ID NO: 5485), GUGgucauua (SEQ ID NO: 5486), UAGgugaaag (SEQ ID NO: 5487), ACUguaauuc (SEQ ID NO: 5488), GCAguacagg (SEQ ID NO: 5489), UCGgugaguc (SEQ ID NO: 5490), UAUguaggga (SEQ ID NO: 5491), AUGguauguc (SEQ ID NO: 5492), GUGgugugug (SEQ ID NO: 5493), CUGgugaccu (SEQ ID NO: 5494), AAUgugaaua (SEQ ID NO: 5495), UAGgucucac (SEQ ID NO: 5496), GAGguuauug (SEQ ID NO: 5497), UGAguaggcu (SEQ ID NO: 5498), CGGgcacgua (SEQ ID NO: 5499), GCAguaaaua (SEQ ID NO: 5500), CCGgugagag (SEQ ID NO: 5501), UAAguugguc (SEQ ID NO: 5502), CCGgugagcc (SEQ ID NO: 5503), AAGguuguca (SEQ ID NO: 5504), CUGguauuau (SEQ ID NO: 5505), GGGguauggg (SEQ ID NO: 5506), AAAgucagua (SEQ ID NO: 5507), UUUguaugua (SEQ ID NO: 5508), UAAguacugc (SEQ ID NO: 5509), CAGguaccaa (SEQ ID NO: 5510), GAAguucaga (SEQ ID NO: 5511), AUGgugcggu (SEQ ID NO: 5512), GUGgugaggu (SEQ ID NO: 5513), UGAguaagcc (SEQ ID NO: 5514), UAUguaaggg (SEQ ID NO: 5515), GUGguggaaa (SEQ ID NO: 5516), GAGgugauug (SEQ ID NO: 5517), GGAguuugua (SEQ ID NO: 5518), AAGgucacga (SEQ ID NO: 5519), GUGguagagg (SEQ ID NO: 5520), UAAguauauc (SEQ ID NO: 5521), AAGgugucca (SEQ ID NO: 5522), UAUgugguau (SEQ ID NO: 5523), GAGguacaau (SEQ ID NO: 5524), AAGguggggg (SEQ ID NO: 5525), GGAguaggug (SEQ ID NO: 5526), and UAGgugacuu (SEQ ID NO: 5527).

In some embodiments, the splice site sequence (e.g., 5′ splice site sequence) comprises AGA. In some embodiments, the splice site sequence (e.g., 5′ splice site sequence) comprises AAA. In some embodiments, the splice site sequence (e.g., 5′ splice site sequence) comprises AAC. In some embodiments, the splice site sequence (e.g., 5′ splice site sequence) comprises AAU. In some embodiments, the splice site sequence (e.g., 5′ splice site sequence) comprises AAG. In some embodiments, the splice site sequence (e.g., 5′ splice site sequence) comprises ACA. In some embodiments, the splice site sequence (e.g., 5′ splice site sequence) comprises AUA. In some embodiments, the splice site sequence (e.g., 5′ splice site sequence) comprises AUU. In some embodiments, the splice site sequence (e.g., 5′ splice site sequence) comprises AUG. In some embodiments, the splice site sequence (e.g., 5′ splice site sequence) comprises AUC. In some embodiments, the splice site sequence (e.g., 5′ splice site sequence) comprises CAA. In some embodiments, the splice site sequence (e.g., 5′ splice site sequence) comprises CAU. In some embodiments, the splice site sequence (e.g., 5′ splice site sequence) comprises CAC. In some embodiments, the splice site sequence (e.g., 5′ splice site sequence) comprises CAG. In some embodiments, the splice site sequence (e.g., 5′ splice site sequence) comprises GAA. In some embodiments, the splice site sequence (e.g., 5′ splice site sequence) comprises GAC. In some embodiments, the splice site sequence (e.g., 5′ splice site sequence) comprises GAU. In some embodiments, the splice site sequence (e.g., 5′ splice site sequence) comprises GAG. In some embodiments, the splice site sequence (e.g., 5′ splice site sequence) comprises GGA. In some embodiments, the splice site sequence (e.g., 5′ splice site sequence) comprises GCA. In some embodiments, the splice site sequence (e.g., 5′ splice site sequence) comprises GGG. In some embodiments, the splice site sequence (e.g., 5′ splice site sequence) comprises GGC. In some embodiments, the splice site sequence (e.g., 5′ splice site sequence) comprises GUU. In some embodiments, the splice site sequence (e.g., 5′ splice site sequence) comprises GGU. In some embodiments, the splice site sequence (e.g., 5′ splice site sequence) comprises GUC. In some embodiments, the splice site sequence (e.g., 5′ splice site sequence) comprises GUA. In some embodiments, the splice site sequence (e.g., 5′ splice site sequence) comprises GUG. In some embodiments, the splice site sequence (e.g., 5′ splice site sequence) comprises UCU. In some embodiments, the splice site sequence (e.g., 5′ splice site sequence) comprises UCC. In some embodiments, the splice site sequence (e.g., 5′ splice site sequence) comprises UCA. In some embodiments, the splice site sequence (e.g., 5′ splice site sequence) comprises UCG. In some embodiments, the splice site sequence (e.g., 5′ splice site sequence) comprises UUU. In some embodiments, the splice site sequence (e.g., 5′ splice site sequence) comprises UUC. In some embodiments, the splice site sequence (e.g., 5′ splice site sequence) comprises UUA. In some embodiments, the splice site sequence (e.g., 5′ splice site sequence) comprises UUG. In some embodiments, the splice site sequence (e.g., 5′ splice site sequence) comprises UGU. In some embodiments, the splice site sequence (e.g., 5′ splice site sequence) comprises UAU. In some embodiments, the splice site sequence (e.g., 5′ splice site sequence) comprises GGA. In some embodiments, the splice site sequence (e.g., 5′ splice site sequence) comprises CUU. In some embodiments, the splice site sequence (e.g., 5′ splice site sequence) comprises CUC. In some embodiments, the splice site sequence (e.g., 5′ splice site sequence) comprises CUA. In some embodiments, the splice site sequence (e.g., 5′ splice site sequence) comprises CUG. In some embodiments, the splice site sequence (e.g., 5′ splice site sequence) comprises CCU. In some embodiments, the splice site sequence (e.g., 5′ splice site sequence) comprises CCC. In some embodiments, the splice site sequence (e.g., 5′ splice site sequence) comprises CCA. In some embodiments, the splice site sequence (e.g., 5′ splice site sequence) comprises CCG. In some embodiments, the splice site sequence (e.g., 5′ splice site sequence) comprises ACU. In some embodiments, the splice site sequence (e.g., 5′ splice site sequence) comprises ACC. In some embodiments, the splice site sequence (e.g., 5′ splice site sequence) comprises ACG. In some embodiments, the splice site sequence (e.g., 5′ splice site sequence) comprises AGC. In some embodiments, the splice site sequence (e.g., 5′ splice site sequence) comprises AGU. In some embodiments, the splice site sequence (e.g., 5′ splice site sequence) comprises AGG. In some embodiments, the splice site sequence (e.g., 5′ splice site sequence) comprises CGU. In some embodiments, the splice site sequence (e.g., 5′ splice site sequence) comprises UAC. In some embodiments, the splice site sequence (e.g., 5′ splice site sequence) comprises UAA. In some embodiments, the splice site sequence (e.g., 5′ splice site sequence) comprises UAG. In some embodiments, the splice site sequence (e.g., 5′ splice site sequence) comprises CGC. In some embodiments, the splice site sequence (e.g., 5′ splice site sequence) comprises CGA. In some embodiments, the splice site sequence (e.g., 5′ splice site sequence) comprises CGG. In some embodiments, the splice site sequence comprises AGAguaaggg (SEQ ID NO: 667). In some embodiments, the splice site sequence comprises UGAguaagca (SEQ ID NO: 2768).

In an embodiment, a gene sequence or splice site sequence provided herein is related to a proliferative disease, disorder, or condition (e.g., cancer, benign neoplasm, or inflammatory disease). In an embodiment, a gene sequence or splice site sequence provided herein is related to a non-proliferative disease, disorder, or condition. In an embodiment, a gene sequence or splice site sequence provided herein is related to a neurological disease or disorder; autoimmune disease or disorder; immunodeficiency disease or disorder; lysosomal storage disease or disorder; cardiovascular condition, disease or disorder; metabolic disease or disorder; respiratory condition, disease, or disorder; renal disease or disorder; or infectious disease in a subject. In an embodiment, a gene sequence or splice site sequence provided herein is related to a neurological disease or disorder (e.g., Huntington's disease). In an embodiment, a gene sequence or splice site sequence provided herein is related to an immunodeficiency disease or disorder. In an embodiment, a gene sequence or splice site sequence provided herein is related to a lysosomal storage disease or disorder. In an embodiment, a gene sequence or splice site sequence provided herein is related to a cardiovascular condition, disease or disorder. In an embodiment, a gene sequence or splice site sequence provided herein is related to a metabolic disease or disorder. In an embodiment, a gene sequence or splice site sequence provided herein is related to a respiratory condition, disease, or disorder. In an embodiment, a gene sequence or splice site sequence provided herein is related to a renal disease or disorder. In an embodiment, a gene sequence or splice site sequence provided herein is related to an infectious disease.

In an embodiment, a gene sequence or splice site sequence provided herein is related to a mental retardation disorder. In an embodiment, a gene sequence or splice site sequence provided herein is related to a mutation in the SETD5 gene. In an embodiment, a gene sequence or splice site sequence provided herein is related to an immunodeficiency disorder. In an embodiment, a gene sequence and splice site sequence provided herein is related to a mutation in the GATA2 gene. In an embodiment, a gene sequence or splice site sequence provided herein is related to a lysosomal storage disease.

In some embodiments, a compound of Formula (I), (II), (III), (IV) described herein interacts with (e.g., binds to) a splicing complex component (e.g., a nucleic acid (e.g., an RNA) or a protein). In some embodiments, the splicing complex component is selected from 9G8, A1 hnRNP, A2 hnRNP, ASD-1, ASD-2b, ASF, BRR2, B1 hnRNP, C₁ hnRNP, C₂ hnRNP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, LUC7L, M hnRNP, mBBP, muscle-blind like (MBNL), NF45, NFAR, Nova-1, Nova-2, nPTB, P54/SFRS11, polypyrimidine tract binding protein (PTB), a PRP protein (e.g., PRP8, PRP6, PRP31, PRP4, PRP3, PRP28, PRP5, PRP2, PRP19), PRP19 complex proteins, RBM42, R hnRNP, RNPC1, SAD1, SAM68, SC35, SF, SF1/BBP, SF2, SF3A complex, SF3B complex, SFRS10, an Sm protein (such as B, D1, D2, D3, F, E, G), SNU17, SNU66, SNU114, an SR protein, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, Ul snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YBi.

In some embodiments, the splicing complex component comprises RNA (e.g., snRNA).

In some embodiments, a compound described herein binds to a splicing complex component comprising snRNA. The snRNA may be selected from, e.g., U1 snRNA, U2 snRNA, U4 snRNA, U5 snRNA, U6 snRNA, U11 snRNA, U12 snRNA, U4atac snRNA, and any combination thereof.

In some embodiments, the splicing complex component comprises a protein, e.g., a protein associated with an snRNA. In some embodiments, the protein comprises SC35, SRp55, SRp40, SRm300, SFRS10, TASR-1, TASR-2, SF2/ASF, 9G8, SRp75, SRp30c, SRp20 and P54/SFRS11. In some embodiments, the splicing complex component comprises a U2 snRNA auxiliary factor (e.g., U2AF65, U2AF35), Urp/U2AF1-RS2, SF1/BBP, CBP80, CBP 20, SF1 or PTB/hnRNP1. In some embodiments, the hnRNP protein comprises A1, A2/B1, L, M, K, U, F, H, G, R, I or C₁/C₂. Human genes encoding hnRNPs include HNRNPAO, HNRNPAI, HNRNPAILI, HNRNPAIL2, HNRNPA3, HNRNPA2B1, HNRNPAB, HNRNPBI, HNRNPC, HNRNPCLI, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMR1.

In one aspect, the compounds of Formula (I), (II), (III), (IV) and pharmaceutically acceptable salts, solvates, hydrates, tautomers, stereoisomers, and compositions thereof, may modulate (e.g., increase or decrease) a splicing event of a target nucleic acid sequence (e.g., DNA, RNA, or a pre-mRNA), for example, a nucleic acid encoding a gene described herein, or a nucleic acid encoding a protein described herein, or a nucleic acid comprising a splice site described herein. In an embodiment, the splicing event is an alternative splicing event.

In an embodiment, the compound of Formula (I), (II), (III), (IV) or a pharmaceutically acceptable salt, solvate, hydrate, tautomer, stereoisomer, and compositions thereof increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR. In an embodiment, the compound of Formula (I), (II), (III), (IV) or a pharmaceutically acceptable salt, solvate, hydrate, tautomer, stereoisomer, and compositions thereof decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR.

In another aspect, the present disclosure features a method of forming a complex comprising a component of a spliceosome (e.g., a major spliceosome component or a minor spliceosome component), a nucleic acid (e.g., a DNA, RNA, e.g., a pre-mRNA), and a compound of Formula (I), (II), (III), (IV) or a pharmaceutically acceptable salt, solvate, hydrate, tautomer, stereoisomer, or composition thereof, comprising contacting the nucleic acid (e.g., a DNA, RNA, e.g., a pre-mRNA) with said compound of Formula (I), (II), (III), (IV). In an embodiment, the component of a spliceosome is selected from the U1, U2, U4, U5, U6, U11, U12, U4atac, U6atac small nuclear ribonucleoproteins (snRNPs), or a related accessory factor.

In an embodiment, the component of a spliceosome is recruited to the nucleic acid in the presence of the compound of Formula (I), (II), (III), (IV) or a pharmaceutically acceptable salt, solvate, hydrate, tautomer, stereoisomer, or composition thereof.

In another aspect, the present disclosure features a method of altering the conformation of a nucleic acid (e.g., a DNA, RNA, e.g., a pre-mRNA) comprising contacting the nucleic acid with a compound of Formula (I), (II), (III), (IV) or a pharmaceutically acceptable salt, solvate, hydrate, tautomer, stereoisomer, or composition thereof. In an embodiment, the altering comprises forming a bulge or kink in the nucleic acid. In an embodiment, the altering comprises stabilizing a bulge or a kink in the nucleic acid. In an embodiment, the altering comprises reducing a bulge or a kink in the nucleic acid. In an embodiment, the nucleic acid comprises a splice site. In an embodiment, the compound of Formula (I), (II), (III), (IV) interacts with a nucleobase, ribose, or phosphate moiety of a nucleic acid (e.g., a DNA, RNA, e.g., pre-mRNA).

The present disclosure also provides methods for the treatment or prevention of a disease, disorder, or condition. In an embodiment, the disease, disorder or condition is related to (e.g., caused by) a splicing event, such as an unwanted, aberrant, or alternative splicing event. In an embodiment, the disease, disorder or condition comprises a proliferative disease (e.g., cancer, benign neoplasm, or inflammatory disease) or non-proliferative disease. In an embodiment, the disease, disorder, or condition comprises a neurological disease, autoimmune disorder, immunodeficiency disorder, cardiovascular condition, metabolic disorder, lysosomal storage disease, respiratory condition, renal disease, or infectious disease in a subject. In another embodiment, the disease, disorder, or condition comprises a haploinsufficiency disease, an autosomal recessive disease (e.g., with residual function), or a paralogue activation disorder. In another embodiment, the disease, disorder, or condition comprises an autosomal dominant disorder (e.g., with residual function). Such methods comprise the step of administering to the subject in need thereof an effective amount of a compound of Formula (I), (II), (III), (IV), or a pharmaceutically acceptable salt, solvate, hydrate, tautomer, stereoisomer thereof, or a pharmaceutical composition thereof. In certain embodiments, the methods described herein include administering to a subject an effective amount of a compound of Formula (I), (II), (III), (IV), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease may also be associated with inhibition of apoptosis of a cell in a biological sample or subject. All types of biological samples described herein or known in the art are contemplated as being within the scope of the disclosure. The compounds of Formula (I), (II), (III), (IV) and pharmaceutically acceptable salts, solvates, hydrates, tautomers, stereoisomers, and compositions thereof, may induce apoptosis, and therefore, be useful in treating and/or preventing proliferative diseases.

In certain embodiments, the proliferative disease to be treated or prevented using the compounds of Formula (I), (II), (III), (IV) is cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AIL) (e.g., B-cell AIL, T-cell AIL), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a.

Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the cancer is selected from adenoid cystic carcinoma (ACC), acute myelocytic leukemia (AML) (e.g., B-cell AML, T-cell AML), chronic myelocytic leukemia (CML) (e.g., B-cell CML, T-cell CML), non-Hodgkin lymphoma (NHL), Burkitt lymphoma, colorectal cancer (e.g., colon cancer, rectal cancer, colorectal adenocarcinoma), prostate cancer (e.g., prostate adenocarcinoma), ovarian cancer (e.g., cystadenocarcinoma, ovarian embryonal carcinoma, ovarian adenocarcinoma), and myelodysplastic syndrome (MDS).

In some embodiments, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the proliferative disease is associated with angiogenesis. All types of angiogenesis disclosed herein or known in the art are contemplated as being within the scope of the disclosure.

In some embodiments, the compound of Formula (I), (II), (III), (IV) or a pharmaceutically acceptable salt thereof, or compositions comprising such compound or pharmaceutically acceptable salt thereof, is used to prevent or treat a non-proliferative disease.

Exemplary non-proliferative diseases include a neurological disease, autoimmune disorder, immunodeficiency disorder, lysosomal storage disease, cardiovascular condition, metabolic disorder, respiratory condition, inflammatory disease, renal disease, or infectious disease.

In certain embodiments, the non-proliferative disease is a neurological disease. In certain embodiments, the compound of Formula (I), (II), (III), (IV), or a pharmaceutically acceptable salt thereof, or compositions comprising such compound or pharmaceutically acceptable salt thereof, is used to prevent or treat a neurological disease, disorder, or condition. A neurological disease, disorder, or condition may include a neurodegenerative disease, a psychiatric condition, or a musculoskeletal disease. A neurological disease may further include a repeat expansion disease, e.g., which may be characterized by the expansion of a nucleic acid sequence in the genome. For example, a repeat expansion disease includes myotonic dystrophy, amyotrophic lateral sclerosis, Huntington's disease, a trinucleotide repeat disease, or a polyglutamine disorder (e.g., ataxia, fragile X syndrome). In some embodiments, the neurological disease comprises a repeat expansion disease, e.g., Huntington's disease. Additional neurological diseases, disorders, and conditions include Alzheimer's disease, Huntington's chorea, a prion disease (e.g., Creutzfeld-Jacob disease, bovine spongiform encephalopathy, Kuru, or scrapie), a mental retardation disorder (e.g., a disorder caused by a SETD5 gene mutation, e.g., intellectual disability-facial dysmorphism syndrome, autism spectrum disorder), Lewy Body disease, diffuse Lewy body disease (DLBD), dementia, progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, Parkinson's disease, Down's syndrome, multiple system atrophy, spinal muscular atrophy (SMA), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, a demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, Machado-Joseph disease, or a combination thereof. In some embodiments, the neurological disease comprises Friedrich's ataxia or Sturge Weber syndrome. In some embodiments, the neurological disease comprises Huntington's disease. In some embodiments, the neurological disease comprises spinal muscular atrophy. All types of neurological diseases disclosed herein or known in the art are contemplated as being within the scope of the disclosure.

In certain embodiments, the non-proliferative disease is an autoimmune disorder or an immunodeficiency disorder. In certain embodiments, the compound of Formula (I), (II), (III), (IV) or a pharmaceutically acceptable salt thereof, or compositions comprising such compound or pharmaceutically acceptable salt thereof, is used to prevent or treat an autoimmune disease, disorder, or condition, or an immunodeficiency disease, disorder, or condition. Exemplary autoimmune and immunodeficiency diseases, disorders, and conditions include arthritis (e.g., rheumatoid arthritis, osteoarthritis, gout), Chagas disease, chronic obstructive pulmonary disease (COPD), dermatomyositis, diabetes mellitus type 1, endometriosis, Goodpasture's syndrome, Graves' disease, Guillain-Barre syndrome (GBS), Hashiomoto's disease, Hidradenitis suppurativa, Kawasaki disease, ankylosing spondylitis, IgA nephropathy, idiopathic thrombocytopenic purpura, inflammatory bowel disease, Crohn's disease, ulcerative colitis, collagenous colitis, lymphocytic colitis, ischemic colitis, diversion colitis, Behcet's syndrome, infective colitis, indeterminate colitisinterstitial cystitis, lupus (e.g., systemic lupus erythematosus, discoid lupus, drug-induced lupus, neonatal lupus), mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, vasculitis, vitiligo, a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), neutropenia, aplastic anemia, and Wegener's granulomatosis. In some embodiments, the autoimmune or immunodeficiency disorder comprises chronic mucocutaneous candidiasis. All types of autoimmune disorders and immunodeficiency disorders disclosed herein or known in the art are contemplated as being within the scope of the disclosure.

In certain embodiments, the non-proliferative disease is a cardiovascular condition. In certain embodiments, the compound of Formula (I), (II), (III), (IV) or a pharmaceutically acceptable salt thereof, or compositions comprising such compound or pharmaceutically acceptable salt thereof, is used to prevent or treat a cardiovascular disease, disorder, or condition.

A cardiovascular disease, disorder, or condition may include a condition relating to the heart or vascular system, such as the arteries, veins, or blood. Exemplary cardiovascular diseases, disorders, or conditions include angina, arrhythmias (atrial or ventricular or both), heart failure, arteriosclerosis, atheroma, atherosclerosis, cardiac hypertrophy, cardiac or vascular aneurysm, cardiac myocyte dysfunction, carotid obstructive disease, endothelial damage after PTCA (percutaneous transluminal coronary angioplasty), hypertension including essential hypertension, pulmonary hypertension and secondary hypertension (renovascular hypertension, chronic glomerulonephritis), myocardial infarction, myocardial ischemia, peripheral obstructive arteriopathy of a limb, an organ, or a tissue; peripheral artery occlusive disease (PAOD), reperfusion injury following ischemia of the brain, heart or other organ or tissue, restenosis, stroke, thrombosis, transient ischemic attack (TIA), vascular occlusion, vasculitis, and vasoconstriction. All types of cardiovascular diseases, disorders, or conditions disclosed herein or known in the art are contemplated as being within the scope of the disclosure.

In certain embodiments, the non-proliferative disease is a metabolic disorder. In certain embodiments, the compound of Formula (I), (II), (III), (IV) or a pharmaceutically acceptable salt thereof, or compositions comprising such compound or pharmaceutically acceptable salt thereof, is used to prevent or treat a metabolic disease, disorder, or condition. A metabolic disease, disorder, or condition may include a disorder or condition that is characterized by abnormal metabolism, such as those disorders relating to the consumption of food and water, digestion, nutrient processing, and waste removal. A metabolic disease, disorder, or condition may include an acid-base imbalance, a mitochondrial disease, a wasting syndrome, a malabsorption disorder, an iron metabolism disorder, a calcium metabolism disorder, a DNA repair deficiency disorder, a glucose metabolism disorder, hyperlactatemia, a disorder of the gut microbiota. Exemplary metabolic conditions include obesity, diabetes (Type I or Type II), insulin resistance, glucose intolerance, lactose intolerance, eczema, hypertension, Hunter syndrome, Krabbe disease, sickle cell anemia, maple syrup urine disease, Pompe disease, and metachromatic leukodystrophy. All types of metabolic diseases, disorders, or conditions disclosed herein or known in the art are contemplated as being within the scope of the disclosure.

In certain embodiments, the non-proliferative disease is a respiratory condition. In certain embodiments, the compound of Formula (I), (II), (III), (IV) or a pharmaceutically acceptable salt thereof, or compositions comprising such compound or pharmaceutically acceptable salt thereof, is used to prevent or treat a respiratory disease, disorder, or condition. A respiratory disease, disorder, or condition can include a disorder or condition relating to any part of the respiratory system, such as the lungs, alveoli, trachea, bronchi, nasal passages, or nose. Exemplary respiratory diseases, disorders, or conditions include asthma, allergies, bronchitis, allergic rhinitis, chronic obstructive pulmonary disease (COPD), lung cancer, oxygen toxicity, emphysema, chronic bronchitis, and acute respiratory distress syndrome. All types of respiratory diseases, disorders, or conditions disclosed herein or known in the art are contemplated as being within the scope of the disclosure.

In certain embodiments, the non-proliferative disease is a renal disease. In certain embodiments, the compound of Formula (I), (II), (III), (IV) or a pharmaceutically acceptable salt thereof, or compositions comprising such compound or pharmaceutically acceptable salt thereof, is used to prevent or treat a renal disease, disorder, or condition. A renal disease, disorder, or condition can include a disease, disorder, or condition relating to any part of the waste production, storage, and removal system, including the kidneys, ureter, bladder, urethra, adrenal gland, and pelvis. Exemplary renal diseases include acute kidney failure, amyloidosis, Alport syndrome, adenovirus nephritis, acute lobar nephronia, tubular necrosis, glomerulonephritis, kidney stones, urinary tract infections, chronic kidney disease, polycystic kidney disease, and focal segmental glomerulosclerosis (FSGS). In some embodiments, the renal disease, disorder, or condition comprises HIV-associated nephropathy or hypertensive nephropathy. All types of renal diseases, disorders, or conditions disclosed herein or known in the art are contemplated as being within the scope of the disclosure.

In certain embodiments, the non-proliferative disease is an infectious disease. In certain embodiments, the compound of Formula (I), (II), (III), (IV) or a pharmaceutically acceptable salt thereof, or compositions comprising such compound or pharmaceutically acceptable salt thereof, is used to prevent or treat an infectious disease, disorder, or condition. An infectious disease may be caused by a pathogen such as a virus or bacteria. Exemplary infectious diseases include human immunodeficiency syndrome (HIV), acquired immunodeficiency syndrome (AIDS), meningitis, African sleeping sickness, actinomycosis, pneumonia, botulism, chlamydia, Chagas disease, Colorado tick fever, cholera, typhus, giardiasis, food poisoning, ebola hemorrhagic fever, diphtheria, Dengue fever, gonorrhea, streptococcal infection (e.g., Group A or Group B), hepatitis A, hepatitis B, hepatitis C, herpes simplex, hookworm infection, influenza, Epstein-Barr infection, Kawasaki disease, kuru, leprosy, leishmaniasis, measles, mumps, norovirus, meningococcal disease, malaria, Lyme disease, listeriosis, rabies, rhinovirus, rubella, tetanus, shingles, scarlet fever, scabies, Zika fever, yellow fever, tuberculosis, toxoplasmosis, or tularemia. In some embodiments, the infectious disease comprises cytomegalovirus. All types of infectious diseases, disorders, or conditions disclosed herein or known in the art are contemplated as being within the scope of the disclosure.

In certain embodiments, the disease, disorder, or condition is a haploinsufficiency disease. In certain embodiments, the compound of Formula (I), (II), (III), (IV) or a pharmaceutically acceptable salt thereof, or compositions comprising such compound or pharmaceutically acceptable salt thereof, is used to prevent or treat a haploinsufficiency disease, disorder, or condition. A haploinsufficiency disease, disorder, or condition may refer to a monogenic disease in which an allele of a gene has a loss-of-function lesion, e.g., a total loss of function lesion. In an embodiment, the loss-of-function lesion is present in an autosomal dominant inheritance pattern or is derived from a sporadic event. In an embodiment, the reduction of gene product function due to the altered allele drives the disease phenotype despite the remaining functional allele (i.e. said disease is haploinsufficient with regard to the gene in question). In an embodiment, a compound of Formula (I), (II), (III), (IV) increases expression of the haploinsufficient gene locus. In an embodiment, a compound of Formula (I), (II), (III), (IV) increases one or both alleles at the haploinsufficient gene locus. Exemplary haploinsufficiency diseases, disorders, and conditions include Robinow syndrome, cardiomyopathy, cerebellar ataxia, pheochromocytoma, Charcot-Marie-Tooth disease, neuropathy, Takenouchi-Kosaki syndrome, Coffin-Siris syndrome 2, chromosome lp35 deletion syndrome, spinocerebellar ataxia 47, deafness, seizures, dystonia 9, GLUT1 deficiency syndrome 1, GLUT1 deficiency syndrome 2, stomatin-deficient cryohydrocytosis, basal cell carcinoma, basal cell nevus syndrome, medulloblastoma, somatic, brain malformations, macular degeneration, cone-rod dystrophy, Dejerine-Sottas disease, hypomyelinating neuropathy, Roussy-Levy syndrome, glaucoma, autoimmune lymphoproliferative syndrome, pituitary hormone deficiency, epileptic encephalopathy, early infantile, popliteal pterygium syndrome, van der Woude syndrome, Loeys-Dietz syndrome, Skraban-Deardorff syndrome, erythrocytosis, megalencephaly-polymicrogyria-polydactyly-hydrocephalus syndrome, mental retardation, CINCA syndrome, familial cold inflammatory syndrome 1, keratoendothelitis fugax hereditaria, Muckle-Wells syndrome, Feingold syndrome 1, Acute myeloid leukemia, Heyn-Sproul-Jackson syndrome, Tatton-Brown-Rahman syndrome, Shashi-Pena syndrome, Spastic paraplegia, autosomal dominant, macrophthalmia, colobomatous, with microcornea, holoprosencephaly, schizencephaly, endometrial cancer, familial, colorectal cancer, hereditary nonpolyposis, intellectual developmental disorder with dysmorphic facies and behavioral abnormalities, ovarian hyperstimulation syndrome, schizophrenia, Dias-Logan syndrome, premature ovarian failure, dystonia, dopa-responsive, due to sepiapterin reductase deficiency, Beck-Fahrner syndrome, chromosome 2p12-p11.2 deletion syndrome, neuronopathy, spastic paraplegia, familial adult myoclonic, colorectal cancer, hypothyroidism, Culler-Jones syndrome, holoprosencephaly, myelokathexis, WHIM syndrome, Mowat-Wilson syndrome, mental retardation, an intellectual developmental disorder, autism spectrum disorder, epilepsy, epileptic encephalopathy, Dravet syndrome, migraines, a mental retardation disorder (e.g., a disorder caused by a SETD5 gene mutation, e.g., intellectual disability-facial dysmorphism syndrome, autism spectrum disorder), a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), and febrile seizures.

In certain embodiments, the disease, disorder, or condition is an autosomal recessive disease, e.g., with residual function. In certain embodiments, the compound of Formula (I), (II), (III), (IV), or a pharmaceutically acceptable salt thereof, or compositions comprising such compound or pharmaceutically acceptable salt thereof, is used to prevent or treat an autosomal recessive disease, disorder, or condition. An autosomal recessive disease with residual function may refer to a monogenic disease with either homozygous recessive or compound heterozygous heritability. These diseases may also be characterized by insufficient gene product activity (e.g., a level of gene product greater than 0%). In an embodiment, a compound of Formula (I), (II), (III), (IV) may increase the expression of a target (e.g., a gene) related to an autosomal recessive disease with residual function. Exemplary autosomal recessive diseases with residual function include Friedreich's ataxia, Stargardt disease, Usher syndrome, chlorioderma, fragile X syndrome, achromatopsia 3, Hurler syndrome, hemophilia B, alpha-1-antitrypsin deficiency, Gaucher disease, X-linked retinoschisis, Wiskott-Aldrich syndrome, mucopolysaccharidosis (Sanfilippo B), DDC deficiency, epidermolysis bullosa dystrophica, Fabry disease, metachromatic leukodystrophy, and odontochondrodysplasia.

In certain embodiments, the disease, disorder, or condition is an autosomal dominant disease. In certain embodiments, the compound of Formula (I), (II), (III), (IV), or a pharmaceutically acceptable salt thereof, or compositions comprising such compound or pharmaceutically acceptable salt thereof, is used to prevent or treat an autosomal dominant disease, disorder, or condition. An autosomal dominant disease may refer to a monogenic disease in which the mutated gene is a dominant gene. These diseases may also be characterized by insufficient gene product activity (e.g., a level of gene product greater than 0%). In an embodiment, a compound of Formula (I), (II), (III), (IV) may increase the expression of a target (e.g., a gene) related to an autosomal dominant disease. Exemplary autosomal dominant diseases include Huntington's disease, achondroplasia, antithrombin III deficiency, Gilbert's disease, Ehlers-Danlos syndrome, hereditary hemorrhagic telangiectasia, intestinal polyposis, hereditary elliptosis, hereditary spherocytosis, marble bone disease, Marfan's syndrome, protein C deficiency, Treacher Collins syndrome, Von Willebrand's disease, tuberous sclerosis, osteogenesis imperfecta, polycystic kidney disease, neurofibromatosis, and idiopathic hypoparathyroidism.

In certain embodiments, the disease, disorder, or condition is a paralogue activation disorder. In certain embodiments, the compound of Formula (I), (II), (III), (IV), or a pharmaceutically acceptable salt thereof, or compositions comprising such compound or pharmaceutically acceptable salt thereof, is used to prevent or treat a paralogue activation disease, disorder, or condition. A paralogue activation disorder may comprise a homozygous mutation of genetic locus leading to loss-of-function for the gene product. In these disorders, there may exist a separate genetic locus encoding a protein with overlapping function (e.g. developmental paralogue), which is otherwise not expressed sufficiently to compensate for the mutated gene. In an embodiment, a compound of Formula (I), (II), (III), (IV) activates a gene connected with a paralogue activation disorder (e.g., a paralogue gene).

The cell described herein may be an abnormal cell. The cell may be in vitro or in vivo. In certain embodiments, the cell is a proliferative cell. In certain embodiments, the cell is a cancer cell. In certain embodiments, the cell is a non-proliferative cell. In certain embodiments, the cell is a blood cell. In certain embodiments, the cell is a lymphocyte. In certain embodiments, the cell is a benign neoplastic cell. In certain embodiments, the cell is an endothelial cell. In certain embodiments, the cell is an immune cell. In certain embodiments, the cell is a neuronal cell. In certain embodiments, the cell is a glial cell. In certain embodiments, the cell is a brain cell. In certain embodiments, the cell is a fibroblast. In certain embodiment, the cell is a primary cell, e.g., a cell isolated from a subject (e.g., a human subject).

In some embodiments, a compound of Formula (I), (II), (III), or (IV) or a pharmaceutically acceptable salt thereof, e.g., as described herein, has improved cell permeability over a reference compound, e.g., in a standard assay for measuring cell permeability. Cell permeability may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I), (II), (III), or (IV) or a pharmaceutically acceptable salt thereof, e.g., as described herein, has a cell permeability measurement (Papp) of <2×10⁻⁶ cm s⁻¹. In an embodiment, a compound of Formula (I), (II), (III), or (IV) or a pharmaceutically acceptable salt thereof, e.g., as described herein, has a cell permeability measurement (Papp) of between 2-6×10⁻⁶ cm s⁻¹. In an embodiment, a compound of Formula (I), (II), (III), or (IV) or a pharmaceutically acceptable salt thereof, e.g., as described herein, has a cell permeability measurement (Papp) of Papp greater than 6×10⁻⁶ cm s⁻¹. In an embodiment, a compound of Formula (I), (II), (III), or (IV) or a pharmaceutically acceptable salt thereof, e.g., as described herein, has a cell permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), (III), or (IV) or a pharmaceutically acceptable salt thereof, e.g., as described herein, exhibits decreased cell efflux, e.g., over a reference compound, e.g., in a standard assay for measuring cell efflux. Cell efflux may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I), (II), (III), or (IV) or a pharmaceutically acceptable salt thereof, e.g., as described herein, has a cell efflux ratio of less than 1.5. In an embodiment, a compound of Formula (I), (II), (III), or (IV) or a pharmaceutically acceptable salt thereof, e.g., as described herein, has a cell efflux ratio of between 1.5 and 5. In an embodiment, a compound of Formula (I), (II), (III), or (IV) or a pharmaceutically acceptable salt thereof, e.g., as described herein, has a cell efflux ratio greater than 5. In an embodiment, a compound of Formula (I), (II), (III), or (IV) or a pharmaceutically acceptable salt thereof, e.g., as described herein, has a cell efflux ratio less than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), (III), or (IV) or a pharmaceutically acceptable salt thereof, e.g., as described herein, modulates the expression of a target protein (e.g., HTT or MYB) in a reference cell or sample. In an embodiment, a compound of Formula (I), (II), (III), or (IV) or a pharmaceutically acceptable salt thereof, e.g., as described herein, increases the expression of a target protein (e.g., HTT or MYB) in a reference cell or sample. In an embodiment, a compound of Formula (I), (II), (III), or (IV) or a pharmaceutically acceptable salt thereof, e.g., as described herein, decreases the expression of a target protein (e.g., HTT or MYB) in a reference cell or sample. The effect of an exemplary compound of Formula (I), (II), (III), or (IV) on protein abundance may be measured using a standard assay for measuring protein abundance, such as the HiBit-assay system (Promega). In this assay, percent response for each respective cell line may be as calculated at each compound concentration as follows: % response=100*(S−PC)/(NC−PC). For the normalized response at each concentration, a four-parameter logistical regression may be fit to the data and the response may be interpolated at the 50% value to determine a concentration for protein abundance at 50% (IC₅₀) an untreated control. In an embodiment, a compound of Formula (I), (II), (III), or (IV) or a pharmaceutically acceptable salt thereof, e.g., as described herein, has a protein abundance response less than 100 nM. In an embodiment, a compound of Formula (I), (II), (III), or (IV) or a pharmaceutically acceptable salt thereof, e.g., as described herein, has a protein abundance response between 100-1000 nM. In an embodiment, a compound of Formula (I), (II), (III), or (IV) or a pharmaceutically acceptable salt thereof, e.g., as described herein, has a protein abundance response greater than 1000 nM. In an embodiment, a compound of Formula (I), (II), (III), or (IV) or a pharmaceutically acceptable salt thereof, e.g., as described herein, has a protein abundance response greater than 10 uM. In an embodiment, a compound of Formula (I), (II), (III), or (IV) or a pharmaceutically acceptable salt thereof, e.g., as described herein, modulates the protein abundance of a target protein by about 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), (III), or (IV) or a pharmaceutically acceptable salt thereof, e.g., as described herein, modulates the viability of a target cell in a subject or sample. In an embodiment, a compound of Formula (I), (II), (III), or (IV) or a pharmaceutically acceptable salt thereof, e.g., as described herein, increases the viability of a target cell in a subject or sample. In an embodiment, a compound of Formula (I), (II), (III), or (IV) or a pharmaceutically acceptable salt thereof, e.g., as described herein, decreases the viability of a target cell in a subject or sample. In an embodiment, a compound of Formula (I), (II), (III), or (IV) or a pharmaceutically acceptable salt thereof, e.g., as described herein, does not impact the viability of a cell (e.g., is non-toxic) in a subject or sample. The effect an exemplary compound of Formula (I), (II), (III), or (IV) on cell viability may be measured using a standard assay for measuring cell toxicity, such as the Cell Titer Glo 2.0 assay in either K562 (human chronic myelogenous leukemia) or SH-SY5Y (human neuroblastoma) cells. The concentration at which cell viability is measured may be based on the particular assay used. In an embodiment, a compound of Formula (I), (II), (III), or (IV) or a pharmaceutically acceptable salt thereof, e.g., as described herein, is tolerated by a target cell at a concentration of less than 100 nM. In an embodiment, a compound of Formula (I), (II), (III), or (IV) or a pharmaceutically acceptable salt thereof, e.g., as described herein, is tolerated by a target cell at a concentration of between 100-1000 nM. In an embodiment, a compound of Formula (I), (II), (III), or (IV) or a pharmaceutically acceptable salt thereof, e.g., as described herein, is tolerated by a target cell at a concentration of greater than 1000 nM. In an embodiment, a compound of Formula (I), (II), (III), or (IV) or a pharmaceutically acceptable salt thereof, e.g., as described herein, is tolerated by a target cell at a concentration of greater than 10 uM.

In some embodiments, a compound of Formula (I), (II), (III), or (IV) or a pharmaceutically acceptable salt thereof, e.g., as described herein, has improved brain permeability over a reference compound, e.g., in a standard assay for measuring brain permeability. Brain permeability may be measured, for example, by determining the unbound partition coefficient (Kpuu), brain. In such an assay, the unbound brain partition coefficient (K_{p,uu,brain}) may be defined as the ratio of unbound brain-free compound concentration to unbound plasma concentration. It is calculated using the following equation:

$${K}_{p , uu , brain} = \frac{{f}_{u , brain} \times {C}_{brain}}{{f}_{u , plasma} \times {C}_{plasma}}$$

Cᵣₐᵢₙ and Cₚₗₐₛₘₐ represent the total concentrations in brain and plasma, respectively. In this assay, the f_{u,brain} and f_{u,plasma} may be the unbound fraction of the compound in brain and plasma, respectively. Both f_{u,brain} and f_{u,plasma} may be determined in vitro via equilibrium dialysis. In an embodiment, a compound of Formula (I), (II), (III), or (IV) or a pharmaceutically acceptable salt thereof, e.g., as described herein, has a Kp value of greater than 5. In an embodiment, a compound of Formula (I), (II), (III), or (IV) or a pharmaceutically acceptable salt thereof, e.g., as described herein, has a Kp value between 1 and 5. In an embodiment, a compound of Formula (I), (II), (III), or (IV) or a pharmaceutically acceptable salt thereof, e.g., as described herein, has a Kp value between 0.2-1. In an embodiment, a compound of Formula (I), (II), (III), or (IV) or a pharmaceutically acceptable salt thereof, e.g., as described herein, has a Kp value of less than 0.2. In an embodiment, a compound of Formula (I), (II), (III), or (IV) or a pharmaceutically acceptable salt thereof, e.g., as described herein, has a Kpuu value of greater than 2.5. In an embodiment, a compound of Formula (I), (II), (III), or (IV) or a pharmaceutically acceptable salt thereof, e.g., as described herein, has a Kpuu value between 0.5-2.5. In an embodiment, a compound of Formula (I), (II), (III), or (IV) or a pharmaceutically acceptable salt thereof, e.g., as described herein, has a Kpuu value between 0.1-0.5. In an embodiment, a compound of Formula (I), (II), (III), or (IV) or a pharmaceutically acceptable salt thereof, e.g., as described herein, has a Kpuu value of less than 0.1. In an embodiment, a compound of Formula (I), (II), (III), or (IV) or a pharmaceutically acceptable salt thereof, e.g., as described herein, has a brain permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), (III), or (IV) or a pharmaceutically acceptable salt thereof, e.g., as described herein, exhibits selectivity for one target nucleic acid sequence, e.g., pre-mRNA transcript sequence or bulge, compared to another target nucleic acid sequence, e.g., pre-mRNA transcript sequence or bulge. In an embodiment, a compound of Formula (I), (II), (III), or (IV) or a pharmaceutically acceptable salt thereof, e.g., as described herein, exhibits selectivity for HTT, e.g., an HTT-related nucleic acid sequence. In an embodiment, a compound of Formula (I), (II), (III), or (IV) or a pharmaceutically acceptable salt thereof, e.g., as described herein, exhibits selectivity for SMN2, e.g., an SMN2-related nucleic acid sequence. In an embodiment, a compound of Formula (I), (II), (III), or (IV) or a pharmaceutically acceptable salt thereof, e.g., as described herein, exhibits selectivity for Target C, e.g., a Target C-related nucleic acid sequence. In an embodiment, a compound of Formula (I), (II), (III), or (IV) or a pharmaceutically acceptable salt thereof, e.g., as described herein, exhibits selectivity for MYB, e.g., a MYB-related nucleic acid sequence. Selectivity for one target nucleic acid sequence over another may be measured using any number of methods known in the art. In an embodiment, selectivity may be measured by determining the ratio of derived qPCR values (e.g., as described herein) for one target nucleic acid sequence over another. In an embodiment, a compound of Formula (I), (II), (III), or (IV) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for one target nucleic acid sequence over another. In an embodiment, a compound of Formula (I), (II), (III), or (IV) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over another target nucleic acid sequence. In an embodiment, a compound of Formula (I), (II), (III), or (IV) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over another. In an embodiment, a compound of Formula (I), (II), (III), or (IV) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over another target nucleic acid sequence. In an embodiment, a compound of Formula (I), (II), (III), or (IV) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for Target C sequence over another. In an embodiment, a compound of Formula (I), (II), (III), or (IV) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over MYB. In an embodiment, a compound of Formula (I), (II), (III), or (IV) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over HTT. In an embodiment, a compound of Formula (I), (II), (III), or (IV) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over SMN2. In an embodiment, a compound of Formula (I), (II), (III), or (IV) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over HTT. In an embodiment, a compound of Formula (I), (II), (III), or (IV) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over MYB. In an embodiment, a compound of Formula (I), (II), (III), or (IV) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over SMN2. In an embodiment, a compound of Formula (I), (II), (III), or (IV) or a pharmaceutically acceptable salt thereof, e.g., as described herein, has a 3-fold greater selectivity for HTT over MYB. In an embodiment, a compound of Formula (I), (II), (III), or (IV) or a pharmaceutically acceptable salt thereof, e.g., as described herein, has a 3-fold greater selectivity for MYB over HTT. In an embodiment, a compound of Formula (I), (II), (III), or (IV) or a pharmaceutically acceptable salt thereof, e.g., as described herein, has a 10-fold greater selectivity for HTT over MYB. In an embodiment, a compound of Formula (I), (II), (III), or (IV) or a pharmaceutically acceptable salt thereof, e.g., as described herein, has a 10-fold greater selectivity for MYB over HTT. In an embodiment, a compound of Formula (I), (II), (III), or (IV) or a pharmaceutically acceptable salt thereof, e.g., as described herein, has a 3-fold greater selectivity for HTT over SMN2. In an embodiment, a compound of Formula (I), (II), (III), or (IV) or a pharmaceutically acceptable salt thereof, e.g., as described herein, has a 3-fold greater selectivity for SMN2 over HTT. In an embodiment, a compound of Formula (I), (II), (III), or (IV) or a pharmaceutically acceptable salt thereof, e.g., as described herein, has a 10-fold greater selectivity for HTT over SMN2. In an embodiment, a compound of Formula (I), (II), (III), or (IV) or a pharmaceutically acceptable salt thereof, e.g., as described herein, has a 10-fold greater selectivity for SMN2 over HTT. In an embodiment, a compound of Formula (I), (II), (III), or (IV) or a pharmaceutically acceptable salt thereof, e.g., as described herein, has a 3-fold greater selectivity for MYB over SMN2. In an embodiment, a compound of Formula (I), (II), (III), or (IV) or a pharmaceutically acceptable salt thereof, e.g., as described herein, has a 3-fold greater selectivity for SMN2 over MYB. In an embodiment, a compound of Formula (I), (II), (III), or (IV) or a pharmaceutically acceptable salt thereof, e.g., as described herein, has a 10-fold greater selectivity for MYB over SMN2. In an embodiment, a compound of Formula (I), (II), (III), or (IV) or a pharmaceutically acceptable salt thereof, e.g., as described herein, has a 10-fold greater selectivity for SMN2 over MYB. In an embodiment, a compound of Formula (I), (II), (III), or (IV) or a pharmaceutically acceptable salt thereof, e.g., as described herein, has a selectivity for one target nucleic acid sequence that is greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a second nucleic acid sequence.

In certain embodiments, the methods described herein comprise the additional step of administering one or more additional pharmaceutical agents in combination with the compound of Formula (I), (II), (III), (IV), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment the modulation of splicing induced by the inventive compounds or compositions of this disclosure in the biological sample or subject. Thus, the combination of the inventive compounds or compositions and the additional pharmaceutical agent(s) may be useful in treating, for example, a cancer or other disease, disorder, or condition resistant to a treatment using the additional pharmaceutical agent(s) without the inventive compounds or compositions.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein. Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

Proton NMR: ¹H NMR spectra were recorded in CDCl₃ solution in 5-mm o.d. tubes (Wildmad) at 24° C. and were collected on a BRUKER AVANCE NEO 400 at 400 MHz for ¹H.

The chemical shifts (6) are reported relative to tetramethylsilane (TMS=0.00 ppm) and expressed in ppm.

LC/MS: Liquid chromatography-mass spectrometry (LC/MS) was performed on Shimadzu-2020EV using column: Shim-pack XR-ODS (C18, Ø4.6×50 mm, 3 μm, 120 Å, 40° C.) operating in ESI(+) ionization mode; flow rate=1.2 mL/min. Mobile phase=0.05% TFA in water or CH₃CN; or on Shimadzu-2020EV using column: Poroshell HPH—C18 (C18, Ø4.6×50 mm, 3 μm, 120 Å, 40° C.) operating in ESI(+) ionization mode; flow rate=1.2 mL/min. Mobile phase A: Water/5 mM NH₄HCO₃, Mobile phase B: CH₃CN).

Analytical chiral HPLC: Analytical chiral HPLC was performed on a Agilent 1260 using column: CHIRALPAK IG-3 CHIRALPAK IC-3 or CHirALPAK OJ-3, with flow rate=1.2 mL/min. Mobile phase=MTBE(DEA:EtOI=50:50).

Preparative HPLC or Reverse Phase Flash Chromatography purification: prep-HPLC or reverse phase flash purification was performed using one of the following conditions: Condition 1: Column: C18 silica gel; Mobile Phase A: water, Mobile Phase B: acetonitrile; Gradient 1: 10% B to 50% B in 10 min.

Condition 2: Column: Weich Ultimate XB C18 50*250 mm, 10 μm; Mobile Phase A: water (0.1% HCl), Mobile Phase B: acetonitrile; Flow rate: 90 mL/min; Gradient 1: 15% B to 51% B in 12 min.

Condition 3: Column: XBridge Prep OBD Column 19*150 mm, 8 μm; Mobile Phase A: water (0.05% NH₄HCO₃), Mobile Phase B: acetonitrile; Flow rate: 20 mL/min; Gradient 1: 15% B to 40% B in 8 min; Gradient 2: 20% B to 45% B in 8 min.

Condition 4: Column: Weich Ultimate XB, C18 50*250 mm, 10 μm; Mobile Phase A: water (0.1% TFA), Mobile Phase B: acetonitrile; Flow rate: 90 mL/min; Gradient 1: 10% B to 55% B in 15 min; Gradient 2: 11% B to 46% B in 12 min; Gradient 3: 15% B to 51% B in 12 min.

Condition 5: Column: XBridge Prep OBD Column 19*150 mm, 8 μm; Mobile Phase A: water (0.05% NH₄HCO₃), Mobile Phase B: methanol; Flow rate: 20 mL/min; Gradient 1: 50% B to 80% B in 8 min; Gradient 2: 55% B to 85% B in 8 min; Gradient 3: 20% B to 50% B in 8 min.

Condition 6: Column: Kinetex EVO C18 Column, 30*150 mm, 5 μm; Mobile Phase A: Water (10 mmol/L NH₄HCO₃), Mobile Phase B: acetonitrile; Flow rate: 60 mL/min; Gradient 1: 10% B to 56% B in 10 min.

Condition 7: Column, YMC-Actus Triart C18, 30×150 mm, 5 m; Mobile Phase A: Water (10 mmol/L NH₄HCO₃), Mobile Phase B: ACN; Flow rate: 60 mL/min; Gradient 1: 10% B to 55% B in 8 min.

Preparative chiral HPLC: purification by chiral HPLC was performed on a Gilson-GX 281 using column: CHIRALPAK IG-3, CHIRALPAK IC-3 or CHIRALPAK OJ-3.

Condition 1: Column: CHIRALPAK IF, 3*25 cm, 5 m; Mobile Phase A: Hex: DCM (5:1), Mobile Phase B: ethanol (0.1% DEA); Flow rate: 35 mL/min; Gradient 1: 30% B to 30% B in 25 min.

Condition 2: Column: CHIRAL ART Cellulose-SZ, 3*25 cm, 5 m; Mobile Phase A: methanol, Mobile Phase B: DCM (0.1% 2 M NH₃-methanol); Flow rate: 35 mL/min; Gradient 1: 30% B to 30% B in 15 min.

Condition 3: Column: CHIRAL ART Cellulose-SZ, 3*25 cm, 5 m; Mobile Phase A: methanol:DCM=2:1 (0.2% DEA), Mobile Phase B: methanol:DCM=2:1 (0.2% DEA); Flow rate: 35 mL/min; Gradient: 50% B to 50% B in 10 min.

Condition 4: Column: CHIRAL ART Cellulose-SB, 3*25 cm, 5 m; Mobile Phase A: Hex: DCM (1: 1), Mobile Phase B: ethanol (0.1% DEA); Flow rate: 32 mL/min; Gradient 1: 70% B to 70% B in 32 min.

Condition 5: Column: YMC-Actus Triart Diol-HILIC, 3*25 cm, 5 pm; Mobile Phase A: CO₂, Mobile Phase B: methanol (0.1% 2 M NH₃-methanol); Flow rate: 80 mL/min; Gradient 1: isocratic 30% B; Column Temperature (° C.): 35; Back Pressure (bar): 100.

Condition 6: Column: CHIRAL ART Cellulose-SZ, 3*25 cm, Mobile Phase A: Hex: DCM (1: 1), Mobile Phase B: ethanol; Flow rate: 35 mL/min; Gradient: 50% B to 50% B in 12 min.

Condition 7: Column: Xselect CSH C18 OBD Column 30*150 mm, 5 pm; Mobile Phase A: water (0.05% HCl); Mobile Phase B: acetonitrile; Gradient 1: 2 min at 5% B, 5% B to 35% in 8 min.

Condition 8: Column, XBridge Shield RP18 OBD Column, 30*150 mm, 5 pm; Mobile Phase A: water (10 mmol/L NH₄HCO₃), Mobile Phase B: acetonitrile; Gradient 1:15% B to 55% B in 10 min.

Condition 9: Column: YMC-Actus Triart C18, 30×150 mm, 5 m; Mobile Phase A: Water (10 mmol/L NH₄HCO₃), Mobile Phase B: Acetonitrile; Flow rate: 60 mL/min; Gradient 1: 15% B to 49% B in 8 min. Gradient 2: 10% B to 55% B in 8 min.

### General Synthetic Scheme

Compounds of the present disclosure may be prepared using a synthetic protocol illustrated in Schemes A-C below.

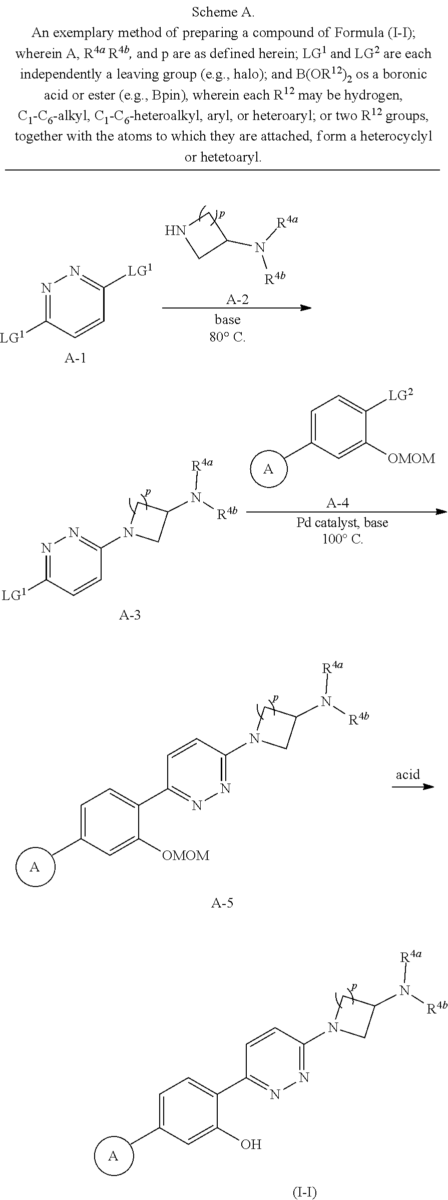

An exemplary method of preparing a compound described herein, such as a compound of Formula (I-I), is provided in Scheme A. In this scheme, A-3 is prepared in Step 1 by incubating A-1 with A-2 in the presence of a base such as potassium carbonate, potassium phosphate, potassium acetate, or a similar reagent. The coupling of A-1 and A-2 may be carried out in acetonitrile, or a similar solvent or mixture, and heated to 80° C. or temperature sufficient to provide A-3.

In Step 2, A-5 is prepared by incubating A-3 with A-4 in the presence of 1,1′-bis(diphenylphosphino)ferrocene)palladium(II) dichloride (Pd(dppf)Cl₂) and tripotassium phosphate (K₃PO₄) or a similar reagent. Alternative catalysts to Pd(dppf)Cl₂ may also be used, such as a suitable palladium catalyst (e.g., a catalyst suitable for a Suzuki reaction), for example, tris(dibenzylideneacetone)-dipalladium(0) (Pd₂(dba)₃), [(2-Di-tert-butylphosphino-2′,4′,6′-triisopropyl-1,1′-biphenyl)-2-(2′-amino-1,1′-biphenyl)]palladium(II) methanesulfonate (^{t}BuXPhos-Pd-G3). The coupling of A-1 and A-2 may be carried out in a mixture of dioxane and water, or a similar solvent or mixture, and heated to 100° C. or temperature sufficient to provide A-3.

In Step 3, the compound of Formula (I-I) is prepared by incubating A-5 with an acid, such as trifluoroacetic acid or HCl, in a solvent such as, dichloromethane at a temperature sufficient to provide the compound of Formula (I-I) or a precursor to a compound of Formula (I-I). A precursor to a compound of Formula (I-I) may be modified to arrive at a compound of Formula (I-I), for example, by removal of protecting groups and/or methylation. Each starting material and/or intermediate in Scheme A may be protected and deprotected using standard protecting group methods. In addition, purification and characterization of each intermediate as well as the final compound of Formula (I) may be afforded by any accepted procedure.

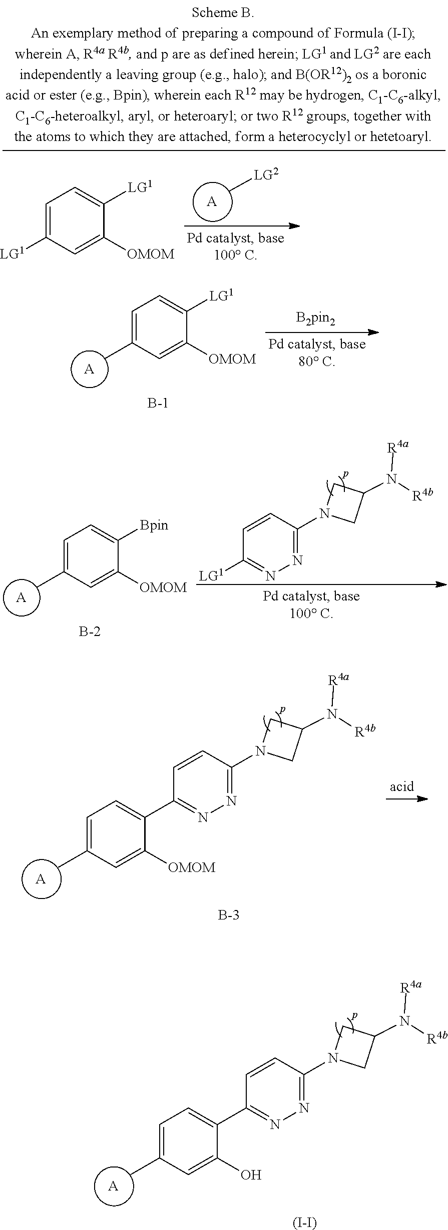

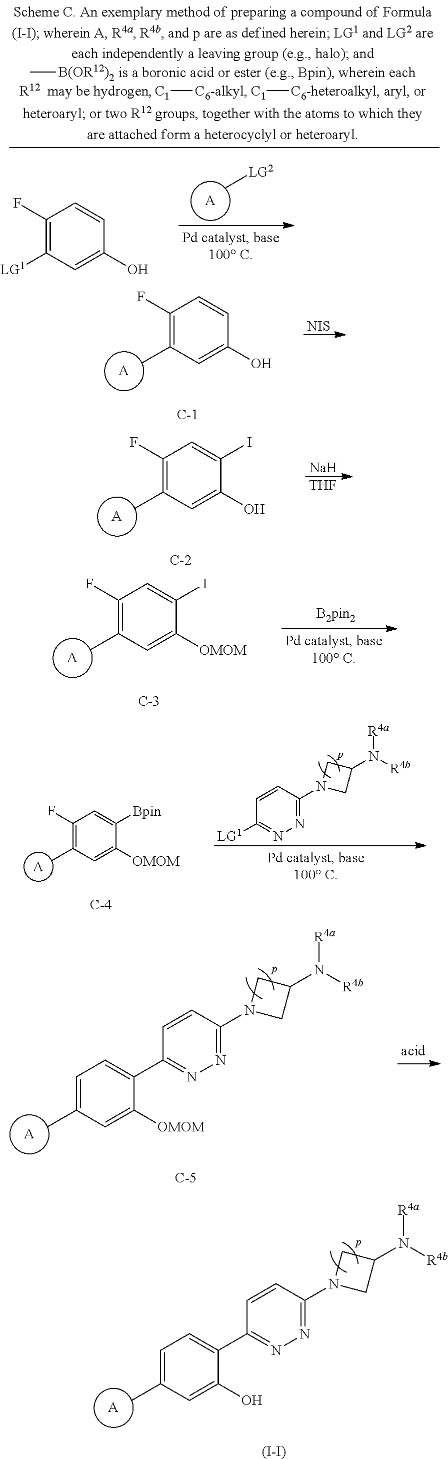

### Example 1: Synthesis of Compound 101

### Synthesis of Intermediate B]

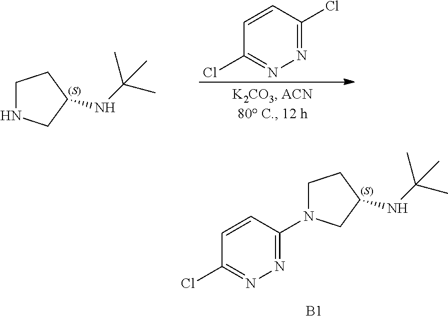

To a stirred mixture of (3S)—N-tert-butylpyrrolidin-3-amine (200 mg, 1.406 mmol, 1 equiv) and 3,6-dichloropyridazine (251.34 mg, 1.687 mmol, 1.2 equiv) in acetonitrile (2 mL) was added K₂CO₃ (582.95 mg, 4.218 mmol, 3 equiv) at room temperature. The resulting mixture was stirred for 12 h at 80° C., then concentrated under reduced pressure to give a residue. The residue was purified by silica gel column chromatography, eluted with PE/EA (1:1) to afford (3S)—N-tert-butyl-1-(6-chloropyridazin-3-yl)pyrrolidin-3-amine (300 mg, 84%) as a solid. LCMS (ES, m/z): 255 [M+H]⁺.

### Synthesis of Intermediate B2

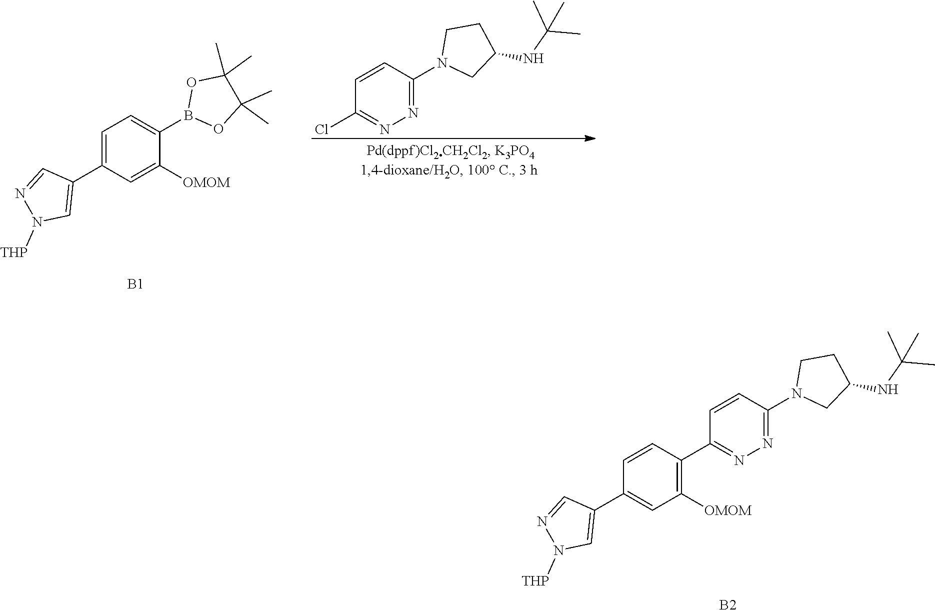

To a stirred mixture of 4-[3-(methoxymethoxy)-4-(4,4,5,5-tetramethyl-1,3,2-dioxaborolan-2-yl)phenyl]-1-(oxan-2-yl)pyrazole (160 mg, 0.386 mmol, 1 equiv) and (3S)—N-tert-butyl-1-(6-chloropyridazin-3-yl)pyrrolidin-3-amine (98.38 mg, 0.386 mmol, 1.0 equiv) in 1,4-dioxane (2.5 mL) and H₂O (0.5 mL) was added K₃PO₄ (245.92 mg, 1.158 mmol, 3 equiv) and Pd(dppf)Cl₂·CH₂Cl₂ (31.46 mg, 0.039 mmol, 0.1 equiv) at room temperature under nitrogen atmosphere. The resulting mixture was stirred for 3 h at 100° C. under nitrogen atmosphere, then concentrated under reduced pressure to give a residue. The residue was purified by silica gel column chromatography, eluted with DCM/MeOH (10:1) to afford (3S)—N-tert-butyl-1-{6-[2-(methoxymethoxy)-4-[1-(oxan-2-yl)pyrazol-4-yl]phenyl]pyridazin-3-yl}pyrrolidin-3-amine (110 mg, 56%) as a solid. LCMS (ES, m/z): 507 [M+H]⁺.

### Synthesis of Compound 101

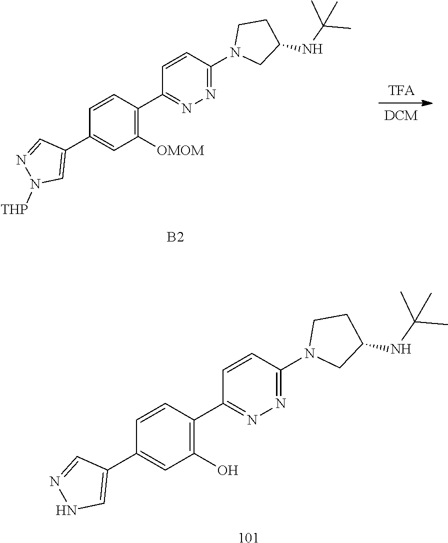

To a stirred mixture of (3S)—N-tert-butyl-1-{6-[2-(methoxymethoxy)-4-[1-(oxan-2-yl)pyrazol-4-yl]phenyl]pyridazin-3-yl}pyrrolidin-3-amine (110 mg, 0.217 mmol, 1 equiv) in DCM (1 mL) was added TFA (1 mL) at room temperature. The resulting mixture was stirred for 1 h at room temperature, then concentrated under reduced pressure to give a residue. The residue was purified by Prep-HPLC (Condition 3, Gradient 1), followed by chiral HPLC (Condition 1, Gradient 1) to afford 2-{6-[(3S)-3-(tert-butylamino)pyrrolidin-1-yl]pyridazin-3-yl}-5-(1H-pyrazol-4-yl)phenol (16.1 mg, 20%) as a solid. LCMS (ES, m/z): 379 [M+H]⁺. ¹H NMR (400 MHz, Methanol-d₄) δ 8.11 (d, J=9.9 Hz, 1H), 8.01 (s, 2H), 7.80-7.73 (m, 1H), 7.23-7.16 (m, 2H), 7.16 (d, J=9.8 Hz, 1H), 3.90 (dd, J=10.3, 7.0 Hz, 1H), 3.79-3.58 (m, 2H), 3.57-3.46 (m, 1H), 3.24 (dd, J=10.3, 7.4 Hz, 1H), 2.38 (dtd, J=12.9, 6.8, 3.1 Hz, 1H), 2.01-1.86 (m, 1H), 1.23 (s, 9H).

### Example 2: Synthesis of Compound 103

### Synthesis of Intermediate B3

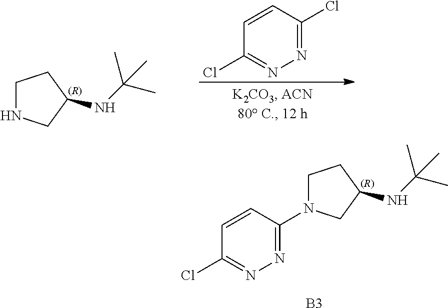

To a mixture of (3R)—N-tert-butylpyrrolidin-3-amine (200 mg, 1.406 mmol, 1 equiv) and 3,6-dichloropyridazine (251.34 mg, 1.687 mmol, 1.2 equiv) in acetonitrile (2 mL) was added K₂CO₃(582.95 mg, 4.218 mmol, 3 equiv) at room temperature. The resulting mixture was stirred for 12 h at 80° C. The resulting mixture was concentrated under reduced pressure to give a residue. The residue was purified by silica gel column chromatography, eluted with PE/EA (1:1) to afford (3R)—N-tert-butyl-1-(6-chloropyridazin-3-yl)pyrrolidin-3-amine (200 mg, 56%) as a solid.

LCMS (ES, m/z): 255[M+H]⁺.

### Synthesis of Intermediate B4

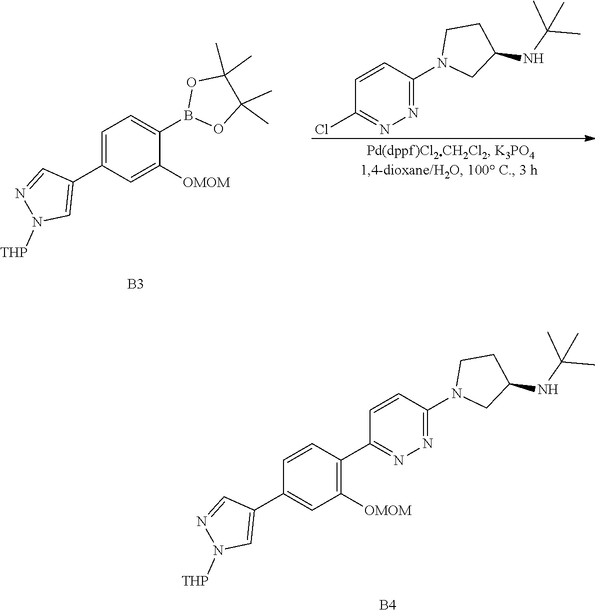

To a stirred mixture of 4-[3-(methoxymethoxy)-4-(4,4,5,5-tetramethyl-1,3,2-dioxaborolan-2-yl)phenyl]-1-(oxan-2-yl)pyrazole (160 mg, 0.386 mmol, 1 equiv) and (3R)—N-tert-butyl-1-(6-chloropyridazin-3-yl)pyrrolidin-3-amine (98.38 mg, 0.386 mmol, 1.0 equiv) in 1,4-dioxane (2 mL) and H₂O (0.4 mL) was added K₃PO₄ (245.92 mg, 1.158 mmol, 3 equiv) and Pd(dppf)Cl₂·CH₂Cl₂ (31.46 mg, 0.039 mmol, 0.1 equiv) at room temperature under nitrogen atmosphere. The resulting mixture was stirred for 3 h at 100° C. under nitrogen atmosphere, then concentrated under reduced pressure to give a residue. The residue was purified by silica gel column chromatography, eluted with DCM/MeOH (10:1) to afford (3R)—N-tert-butyl-1-{6-[2-(methoxymethoxy)-4-[1-(oxan-2-yl)pyrazol-4-yl]phenyl]pyridazin-3-yl}pyrrolidin-3-amine (110 mg, 56%) as a solid. LCMS (ES, m/z): 507[M+H]⁺.

### Synthesis of Compound 103

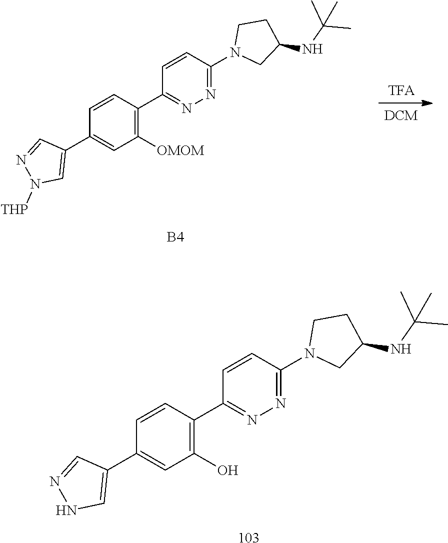

To a stirred solution of (3R)—N-tert-butyl-1-{6-[2-(methoxymethoxy)-4-[1-(oxan-2-yl)pyrazol-4-yl]phenyl]pyridazin-3-yl}pyrrolidin-3-amine (110 mg, 0.217 mmol, 1 equiv) in DCM (1 mL) was added TFA (1 mL) at room temperature. The resulting mixture was stirred for 1 h at room temperature, then concentrated under reduced pressure to give a residue. The residue was purified by Prep-HPLC (Condition 3, Gradient 1), followed by chiral HPLC (Condition 1, Gradient 1) to afford 2-{6-[(3R)-3-(tert-butylamino)pyrrolidin-1-yl]pyridazin-3-yl}-5-(1H-pyrazol-4-yl)phenol (27.6 mg, 34%) as a solid. LCMS (ES, m/z): 379 [M+H]⁺. ¹H NMR (400 MHz, Methanol-d₄) δ 8.11 (d, J=9.7 Hz, 1H), 8.04-7.99 (m, 2H), 7.77 (d, J=8.8 Hz, 1H), 7.23-7.13 (m, 3H), 3.91 (dd, J=10.3, 7.0 Hz, 1H), 3.72 (dd, J=13.1, 8.0 Hz, 2H), 3.58-3.49 (m, 1H), 3.26 (dd, J=10.3, 7.4 Hz, 1H), 2.39 (s, 1H), 2.02-1.88 (m, 1H), 1.24 (s, 9H).

### Example 3: Synthesis of Compound 209

### Synthesis of Intermediate B5

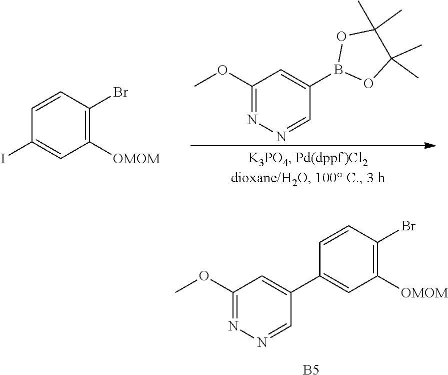

To a stirred mixture of 1-bromo-4-iodo-2-(methoxymethoxy)benzene (4 g, 11.663 mmol, 1 equiv) and 3-methoxy-5-(4,4,5,5-tetramethyl-1,3,2-dioxaborolan-2-yl)pyridazine (3.03 g, 12.829 mmol, 1.1 equiv) in dioxane (40 mL) and H₂O (4 mL) was added K₃PO₄ (7.43 g, 34.989 mmol, 3 equiv) and Pd(dppf)Cl₂ (0.85 g, 1.166 mmol, 0.1 equiv) at room temperature under nitrogen atmosphere. The resulting mixture was stirred for 3 h at 100° C. under nitrogen atmosphere, then concentrated under reduced pressure to give a residue. The residue was purified by silica gel column chromatography, eluted with PE/EA (1:1) to afford 5-[4-bromo-3-(methoxymethoxy)phenyl]-3-methoxypyridazine (4.2 g, 89%) as a solid. LCMS (ES, m/z): 325 [M+H]⁺.

### Synthesis of Intermediate B6

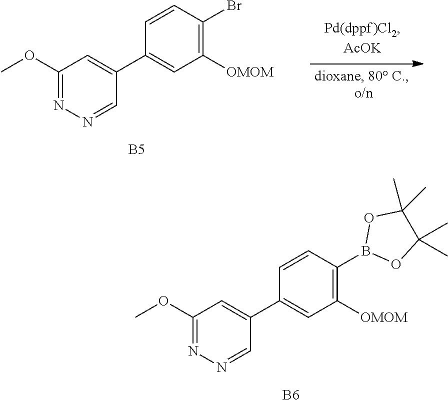

To a stirred mixture of 5-[4-bromo-3-(methoxymethoxy)phenyl]-3-methoxypyridazine (2 g, 6.151 mmol, 1 equiv) and bis(pinacolato)diboron (3.12 g, 12.286 mmol, 2.00 equiv) in dioxane (40 mL) was added AcOK (1.81 g, 18.453 mmol, 3 equiv) and Pd(dppf)Cl₂ (0.45 g, 0.615 mmol, 0.1 equiv) at room temperature under nitrogen atmosphere. The resulting mixture was stirred overnight at 80° C. under nitrogen atmosphere, then concentrated under reduced pressure to give a residue. The residue was purified by silica gel column chromatography, eluted with PE/EA (1:1) to afford 3-methoxy-5-[3-(methoxymethoxy)-4-(4,4,5,5-tetramethyl-1,3,2-dioxaborolan-2-yl)phenyl]pyridazine (1 g, 44%) as an oil. LCMS (ES, m/z): 373 [M+H]⁺.

### Synthesis of Intermediate B7

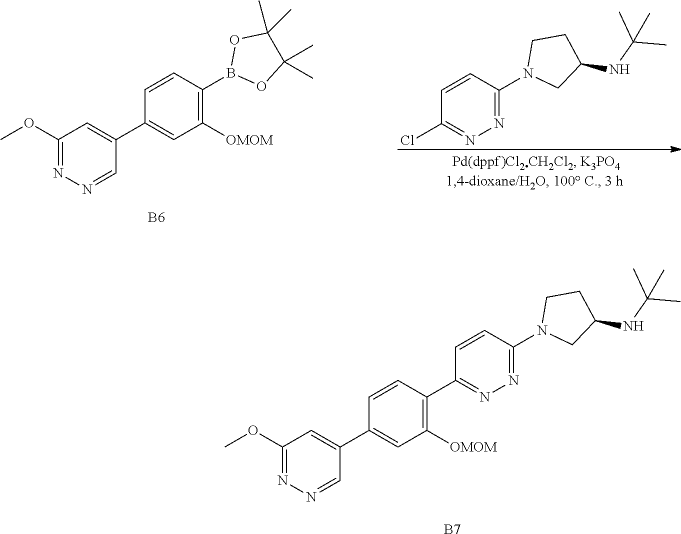

To a stirred mixture of 3-methoxy-5-[3-(methoxymethoxy)-4-(4,4,5,5-tetramethyl-1,3,2-dioxaborolan-2-yl)phenyl]pyridazine (160 mg, 0.430 mmol, 1 equiv) and (3R)—N-tert-butyl-1-(6-chloropyridazin-3-yl)pyrrolidin-3-amine (131.41 mg, 0.516 mmol, 1.2 equiv) in 1,4-dioxane (2.5 mL) and H₂O (0.5 mL) was added K₃PO₄ (182.48 mg, 0.860 mmol, 2 equiv) and Pd(dppf)Cl₂·CH₂Cl₂ (35.02 mg, 0.043 mmol, 0.1 equiv) at room temperature under nitrogen atmosphere. The resulting mixture was stirred for 3 h at 100° C. under nitrogen atmosphere, then concentrated under reduced pressure to give a residue. The residue was purified by silica gel column chromatography, eluted with DCM/MeOH (20:1) to afford (3R)—N-tert-butyl-1-{6-[2-(methoxymethoxy)-4-(6-methoxypyridazin-4-yl)phenyl]pyridazin-3-yl}pyrrolidin-3-amine (60 mg, 30%) as a solid. LCMS (ES, m/z): 465 [M+H]⁺.

### Synthesis of Compound 209

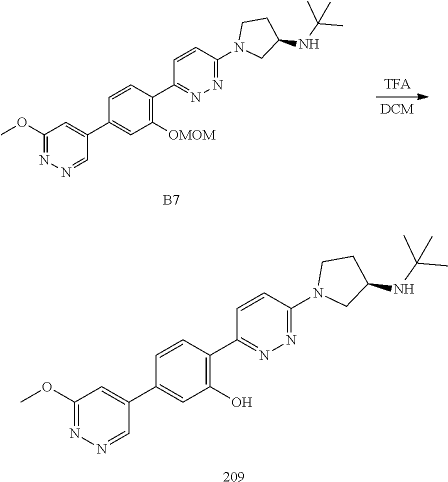

To a stirred solution of (3R)—N-tert-butyl-1-{6-[2-(methoxymethoxy)-4-(6-methoxypyridazin-4-yl)phenyl]pyridazin-3-yl}pyrrolidin-3-amine (60 mg, 0.129 mmol, 1 equiv) in DCM (1 mL) was added TFA (1 mL) at room temperature. The resulting mixture was stirred for 1 h at room temperature, then concentrated under reduced pressure to give a residue. The residue was purified by Chiral HPLC (Condition 2, Gradient 1) to afford 2-{6-[(3R)-3-(tert-butylamino)pyrrolidin-1-yl]pyridazin-3-yl}-5-(6-methoxypyridazin-4-yl)phenol (19.3 mg, 36%) as a solid. LCMS (ES, m/z): 421 [M+H]⁺. ¹H NMR (300 MHz, DMSO-d₆) δ 14.11 (s, 1H), 9.36 (d, J=1.9 Hz, 1H), 8.31 (d, J=9.9 Hz, 1H), 8.05 (d, J=8.2 Hz, 1H), 7.56 (d, J=1.9 Hz, 1H), 7.53-7.43 (m, 2H), 7.19 (d, J=9.8 Hz, 1H), 4.09 (s, 3H), 3.82 (s, 1H), 3.62 (d, J=28.1 Hz, 2H), 3.47 (d, J=9.1 Hz, 1H), 3.13 (s, 1H), 2.22 (s, 1H), 1.79 (s, 1H), 1.11 (s, 9H).

### Example 4: Synthesis of Compound 210

### Synthesis of Intermediate B8

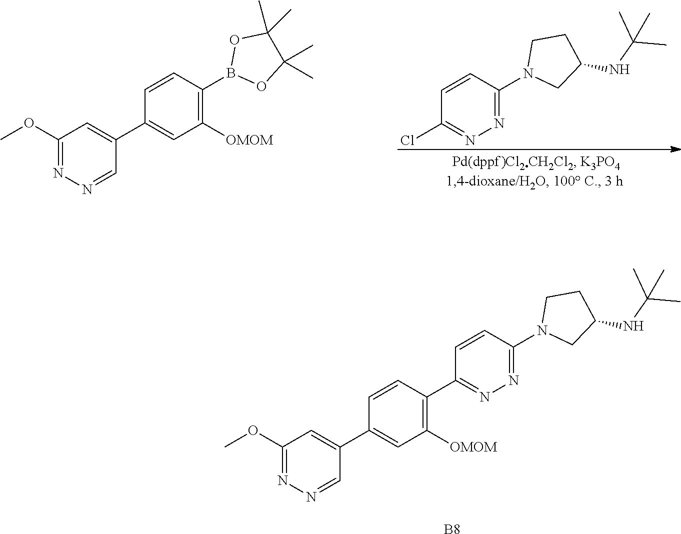

To a stirred mixture of 3-methoxy-5-[3-(methoxymethoxy)-4-(4,4,5,5-tetramethyl-1,3,2-dioxaborolan-2-yl)phenyl]pyridazine (160 mg, 0.430 mmol, 1 equiv) and (3S)—N-tert-butyl-1-(6-chloropyridazin-3-yl)pyrrolidin-3-amine (142.36 mg, 0.559 mmol, 1.3 equiv) in 1,4-dioxane (2.5 mL) and H₂O (0.5 mL) was added K₃PO₄ (273.72 mg, 1.290 mmol, 3 equiv) and Pd(dppf)Cl₂·CH₂Cl₂ (35.02 mg, 0.043 mmol, 0.1 equiv) at room temperature under nitrogen atmosphere. The resulting mixture was stirred for 3 h at 100° C. under nitrogen atmosphere, then concentrated under reduced pressure to give a residue. The residue was purified by silica gel column chromatography, eluted with DCM/MeOH (20:1) to afford (3S)—N-tert-butyl-1-{6-[2-(methoxymethoxy)-4-(6-methoxypyridazin-4-yl)phenyl]pyridazin-3-yl}pyrrolidin-3-amine (60 mg, 30%) as a solid. LCMS (ES, m/z): 465 [M+H]⁺.

### Synthesis of Compound 210

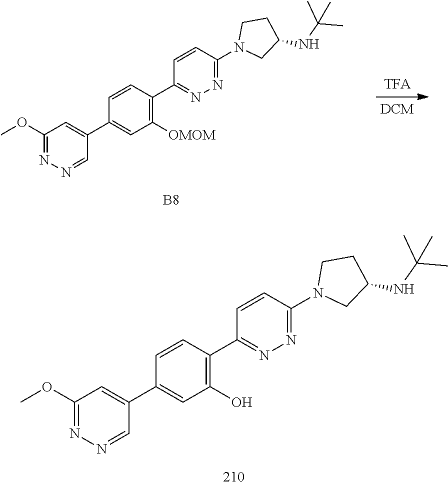

To a stirred solution of (3S)—N-tert-butyl-1-{6-[2-(methoxymethoxy)-4-(6-methoxypyridazin-4-yl)phenyl]pyridazin-3-yl}pyrrolidin-3-amine (60 mg, 0.129 mmol, 1 equiv) in DCM (1 mL) was added TFA (1 mL) at room temperature. The resulting mixture was stirred for 1 h at room temperature, then concentrated under reduced pressure to give a residue. The residue was purified by Chiral HPLC (Condition 2, Gradient 1) to afford 2-{6-[(3S)-3-(tert-butylamino)pyrrolidin-1-yl]pyridazin-3-yl}-5-(6-methoxypyridazin-4-yl)phenol (25.4 mg, 47%) as a solid. LCMS (ES, m/z): 421 [M+H]⁺. ¹H NMR (300 MHz, DMSO-d₆) δ 14.11 (s, 1H), 9.36 (d, J=1.9 Hz, 1H), 8.31 (d, J=9.8 Hz, 1H), 8.05 (d, J=8.2 Hz, 1H), 7.56 (d, J=1.9 Hz, 1H), 7.49 (d, J=9.2 Hz, 2H), 7.20 (d, J=9.6 Hz, 1H), 4.09 (s, 3H), 3.82 (s, 1H), 3.62 (d, J=29.3 Hz, 2H), 3.54-3.42 (m, 1H), 3.13 (s, 1H), 2.21 (s, 1H), 1.80 (s, 1H), 1.11 (s, 9H).

### Example 5: Synthesis of Compound 211

### Synthesis of Intermediate B9

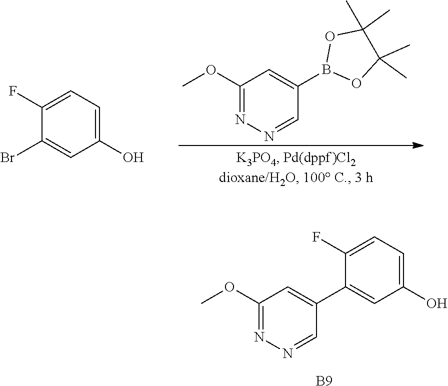

To a stirred mixture of 3-bromo-4-fluorophenol (4 g, 20.943 mmol, 1 equiv) and 3-methoxy-5-(4,4,5,5-tetramethyl-1,3,2-dioxaborolan-2-yl)pyridazine (4.94 g, 20.943 mmol, 1 equiv) in dioxane (60 mL) and H₂O (6 mL) was added K₃PO₄ (4.45 g, 20.943 mmol, 1 equiv) and Pd(dppf)Cl₂ (1.53 g, 2.094 mmol, 0.1 equiv) at room temperature under nitrogen atmosphere.

The resulting mixture was stirred for 3 h at 100° C. under nitrogen atmosphere, then concentrated under reduced pressure to give a residue. The residue was purified by silica gel column chromatography, eluted with PE/EA (1:1) to afford 4-fluoro-3-(6-methoxypyridazin-4-yl)phenol (3 g, 65%) as a solid. LCMS (ES, m/z): 221 [M+H]⁺.

### Synthesis of Intermediate B10

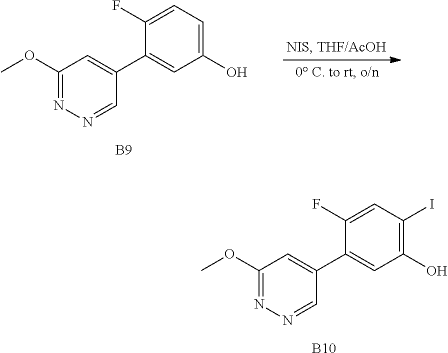

To a stirred mixture of 4-fluoro-3-(6-methoxypyridazin-4-yl)phenol (3.0 g, 13.624 mmol, 1 equiv) in THF (60 mL), AcOH (60 mL), and H₂SO₄ (0.1 mL conc.) was added NIS (3.07 g, 13.624 mmol, 1.0 equiv) in portions at 0° C. The resulting mixture was stirred overnight at room temperature, then concentrated under reduced pressure to give a residue. The residue was purified by reverse phase flash (Condition 4, Gradient 1) to afford 4-fluoro-2-iodo-5-(6-methoxypyridazin-4-yl)phenol (1.8 g, 38%) as a solid. LCMS (ES, m/z): 347 [M+H]⁺.

### Synthesis of Intermediate B11

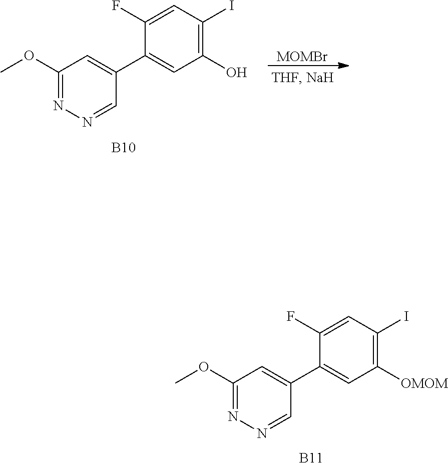

To a solution of 4-fluoro-2-iodo-5-(6-methoxypyridazin-4-yl)phenol (1.4 g, 4.045 mmol, 1 equiv) in THF (14 mL) was added NaH (0.19 g, 4.854 mmol, 1.2 equiv, 60%) at 0° C. The reaction mixture was stirred for 30 min. To the resulting solution was added bromomethyl ether (0.45 g, 3.640 mmol, 0.9 equiv) dropwise at 0° C., then warmed to RT and stirred for 2 h. The reaction mixture was quenched with methanol, then concentrated under reduced pressure to give a residue. The residue was purified by silica gel column chromatography, eluted with PE/EA (5:1) to afford 5-[2-fluoro-4-iodo-5-(methoxymethoxy)phenyl]-3-methoxypyridazine (1 g, 64%) as a solid. LCMS (ES, m/z): 391 [M+H]⁺.

### Synthesis of Intermediate B12

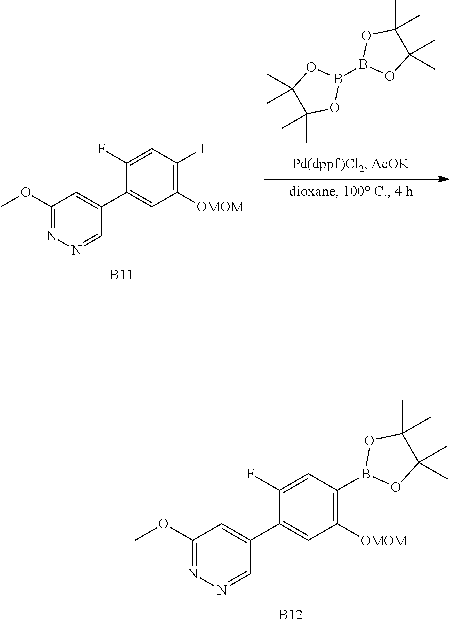

To a stirred mixture of 5-[2-fluoro-4-iodo-5-(methoxymethoxy)phenyl]-3-methoxypyridazine (1 g, 2.563 mmol, 1 equiv) and bis(pinacolato)diboron (2.60 g, 10.252 mmol, 4 equiv) in dioxane (20 mL) was added AcOK (0.75 g, 7.689 mmol, 3 equiv) and Pd(dppf)Cl₂ (0.19 g, 0.256 mmol, 0.1 equiv) at room temperature under nitrogen atmosphere. The resulting mixture was stirred for 4 h at 100° C. under nitrogen atmosphere. The resulting mixture was concentrated under reduced pressure to give a residue. The residue was purified by silica gel column chromatography, eluted with PE/EA (3:1) to afford 5-[2-fluoro-5-(methoxymethoxy)-4-(4,4,5,5-tetramethyl-1,3,2-dioxaborolan-2-yl)phenyl]-3-methoxypyridazine (900 mg, 54%) as an oil. LCMS (ES, m/z): 391[M+H]⁺.

### Synthesis of Intermediate B13

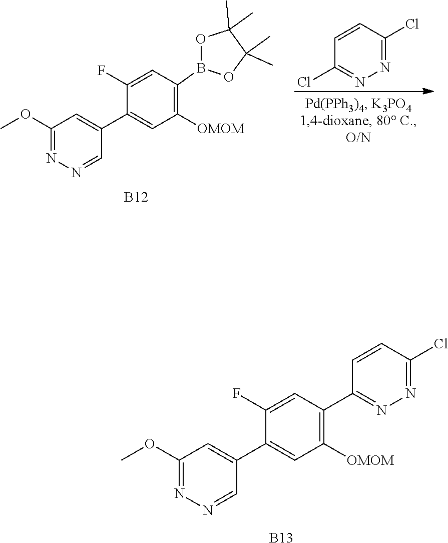

To a stirred mixture of 5-[2-fluoro-5-(methoxymethoxy)-4-(4,4,5,5-tetramethyl-1,3,2-dioxaborolan-2-yl)phenyl]-3-methoxypyridazine (1 g, 1.538 mmol, 1 equiv, 60%) and 3,6-dichloropyridazine (0.30 g, 1.999 mmol, 1.3 equiv) in 1,4-dioxane (20 mL) was added K₃PO₄ (1.37 g, 6.448 mmol, 2 equiv) and Pd(PPh₃)₄(0.18 g, 0.154 mmol, 0.1 equiv) at room temperature under nitrogen atmosphere. The resulting mixture was stirred overnight at 80° C. under nitrogen atmosphere, then concentrated under reduced pressure to give a residue. The residue was purified by silica gel column chromatography, eluted with CH₂Cl₂/MeOH (20:1) to afford 3-chloro-6-[5-fluoro-2-(methoxymethoxy)-4-(6-methoxypyridazin-4-yl)phenyl]pyridazine (470 mg, 81%) as a solid. LCMS (ES, m/z): 377 [M+H]⁺.

### Synthesis of Intermediate B14

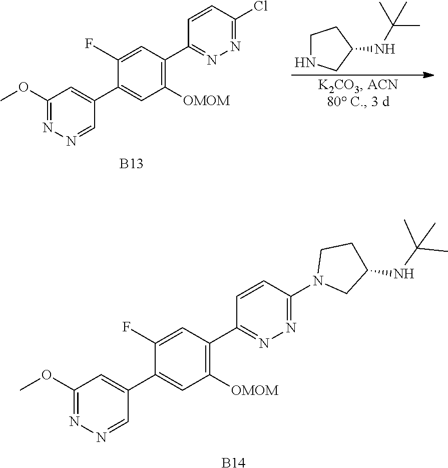

To a stirred mixture of 3-chloro-6-[5-fluoro-2-(methoxymethoxy)-4-(6-methoxypyridazin-4-yl)phenyl]pyridazine (80 mg, 0.212 mmol, 1 equiv) and (3S)—N-tert-butylpyrrolidin-3-amine (36.24 mg, 0.254 mmol, 1.2 equiv) in acetonitrile (1.6 mL) was added K₂CO₃ (88.04 mg, 0.636 mmol, 3 equiv) at room temperature. The resulting mixture was stirred for 3 days at 80° C., then concentrated under reduced pressure to give a residue. The residue was purified by silica gel column chromatography, eluted with CH₂Cl₂/MeOH (10:1) to afford (3S)—N-tert-butyl-1-{6-[5-fluoro-2-(methoxymethoxy)-4-(6-methoxypyridazin-4-yl)phenyl]pyridazin-3-yl}pyrrolidin-3-amine (60 mg, 59%) as a solid. LCMS (ES, m/z): 483 [M+H]⁺.

### Synthesis of Compound 211

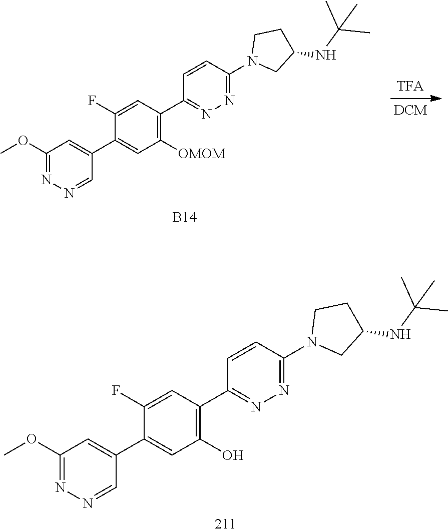

To a stirred solution of (3S)—N-tert-butyl-1-{6-[5-fluoro-2-(methoxymethoxy)-4-(6-methoxypyridazin-4-yl)phenyl]pyridazin-3-yl}pyrrolidin-3-amine (60 mg, 0.124 mmol, 1 equiv) in DCM (1 mL) was added TFA (1 mL) at room temperature. The resulting mixture was stirred for 1 h at room temperature. The resulting mixture was concentrated under reduced pressure to give a residue. The residue was purified by Prep-HPLC (Condition 5, Gradient 1), followed by chiral HPLC (Condition 3, Gradient 1) to afford 2-{6-[(3S)-3-(tert-butylamino)pyrrolidin-1-yl]pyridazin-3-yl}-4-fluoro-5-(6-methoxypyridazin-4-yl)phenol (16.9 mg, 31%) as a solid. LCMS (ES, m/z): 439 [M+H]⁺. ¹H NMR (300 MHz, Methanol-d₄) δ 9.10 (t, J=1.8 Hz, 1H), 8.16 (d, J=9.9 Hz, 1H), 7.78 (d, J=12.3 Hz, 1H), 7.43 (dd, J=1.8, 1.0 Hz, 1H), 7.26 (d, J=6.8 Hz, 1H), 7.19 (d, J=9.7 Hz, 1H), 4.17 (s, 3H), 3.99-3.87 (m, 1H), 3.84-3.63 (m, 2H), 3.62-3.47 (m, 1H), 3.27 (dd, J=10.4, 7.3 Hz, 1H), 2.40 (s, 1H), 2.04-1.85 (m, 1H), 1.23 (s, 9H).

### Example 6: Synthesis of Compound 212

### Synthesis of Intermediate B15

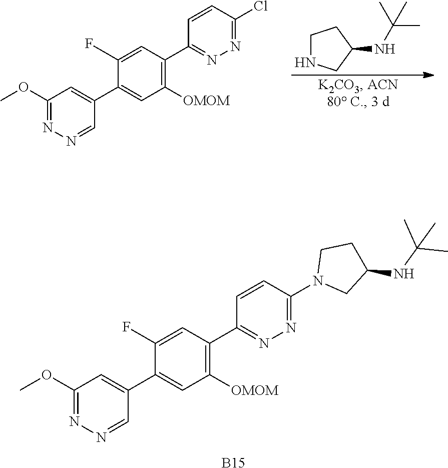

To a stirred mixture of 3-chloro-6-[5-fluoro-2-(methoxymethoxy)-4-(6-methoxypyridazin-4-yl)phenyl]pyridazine (80 mg, 0.212 mmol, 1 equiv) and (3R)—N-tert-butylpyrrolidin-3-amine (36.24 mg, 0.254 mmol, 1.2 equiv) in acetonitrile (1.6 mL) was added K₂CO₃ (88.04 mg, 0.636 mmol, 3 equiv) at room temperature. The resulting mixture was stirred for 2 days at 80° C. The resulting mixture was concentrated under reduced pressure to give a residue. The residue was purified by silica gel column chromatography, eluted with CH₂Cl₂/MeOH (10:1) to afford (3R)—N-tert-butyl-1-{6-[5-fluoro-2-(methoxymethoxy)-4-(6-methoxypyridazin-4-yl)phenyl]pyridazin-3-yl}pyrrolidin-3-amine (60 mg, 59%) as a solid. LCMS (ES, m/z): 483 [M+H]⁺.

### Synthesis of Compound 212

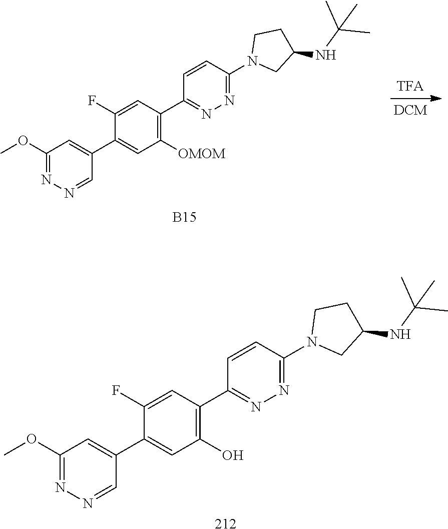

To a stirred solution of (3R)—N-tert-butyl-1-{6-[5-fluoro-2-(methoxymethoxy)-4-(6-methoxypyridazin-4-yl)phenyl]pyridazin-3-yl}pyrrolidin-3-amine (60 mg, 0.124 mmol, 1 equiv) in DCM (1 mL) was added TFA (1 mL) at room temperature. The resulting mixture was stirred for 1 h at room temperature, then concentrated under reduced pressure to give a residue. The residue was purified by Prep-HPLC (Condition 5, Gradient 1), followed by chiral HPLC (Condition 3, Gradient 1) to afford 2-{6-[(3R)-3-(tert-butylamino)pyrrolidin-1-yl]pyridazin-3-yl}-4-fluoro-5-(6-methoxypyridazin-4-yl)phenol (11.8 mg, 22%) as a solid. LCMS (ES, m/z): 439 [M+H]⁺. ¹H NMR (300 MHz, Methanol-d₄) δ 9.10 (t, J=1.8 Hz, 1H), 8.16 (d, J=9.8 Hz, 1H), 7.78 (d, J=12.3 Hz, 1H), 7.43 (d, J=1.4 Hz, 1H), 7.25 (d, J=6.8 Hz, 1H), 7.19 (d, J=9.8 Hz, 1H), 4.17 (s, 3H), 3.93 (s, 1H), 3.72 (dd, J=17.4, 9.0 Hz, 2H), 3.56 (d, J=17.7 Hz, 1H), 3.25 (d, J=8.1 Hz, 1H), 2.39 (s, 1H), 1.95 (dd, J=12.0, 8.6 Hz, 1H), 1.23 (s, 9H).

### Example 7: Synthesis of Compound 213

### Synthesis of Intermediate B16

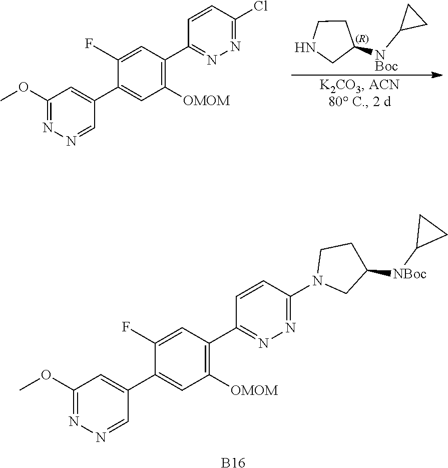

To a stirred solution of 3-chloro-6-[5-fluoro-2-(methoxymethoxy)-4-(6-methoxypyridazin-4-yl)phenyl]pyridazine (80 mg, 0.212 mmol, 1 equiv) and tert-butyl N-cyclopropyl-N-[(3R)-pyrrolidin-3-yl]carbamate (57.67 mg, 0.254 mmol, 1.2 equiv) in acetonitrile (1.6 mL) was added K₂CO₃ (88.04 mg, 0.636 mmol, 3 equiv) at room temperature. The resulting mixture was stirred for 2 days at 80° C., then concentrated under reduced pressure to give a residue. The residue was purified by silica gel column chromatography, eluted with CH₂Cl₂/MeOH (10:1) to afford tert-butyl N-cyclopropyl-N-[(3R)-1-{6-[5-fluoro-2-(methoxymethoxy)-4-(6-methoxypyridazin-4-yl)phenyl]pyridazin-3-yl}pyrrolidin-3-yl]carbamate (60 mg) as a solid. LCMS (ES, m/z): 567 [M+H]⁺.

### Synthesis of Compound 213

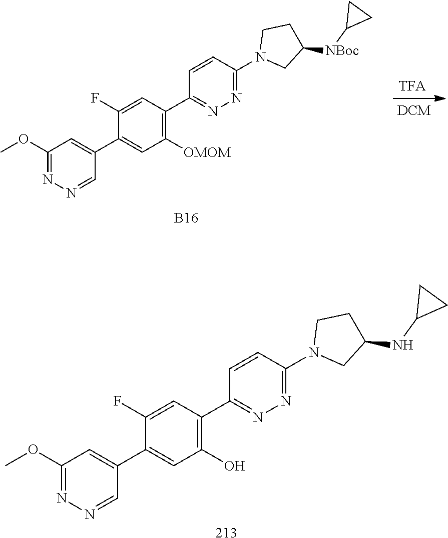

To a stirred solution of tert-butyl N-cyclopropyl-N-[(3R)-1-{6-[5-fluoro-2-(methoxymethoxy)-4-(6-methoxypyridazin-4-yl)phenyl]pyridazin-3-yl}pyrrolidin-3-yl]carbamate (60 mg, 0.106 mmol, 1 equiv) in DCM (1 mL) was added TFA (1 mL) at room temperature. The resulting mixture was stirred for 1 h at room temperature, then concentrated under reduced pressure to give a residue. The residue was purified by Prep-HPLC (Condition 5, Gradient 2) to afford 2-{6-[(3R)-3-(cyclopropylamino)pyrrolidin-1-yl]pyridazin-3-yl}-4-fluoro-5-(6-methoxypyridazin-4-yl)phenol (17.6 mg) as a solid. LCMS (ES, m/z): 423 [M+H]⁺. ¹H NMR (400 MHz, Methanol-d₄) δ 9.10 (t, J=1.8 Hz, 1H), 8.15 (d, J=9.8 Hz, 1H), 7.77 (d, J=12.2 Hz, 1H), 7.42 (s, 1H), 7.25 (d, J=6.8 Hz, 1H), 7.19 (d, J=9.8 Hz, 1H), 4.17 (s, 3H), 3.85 (dd, J=10.8, 6.2 Hz, 1H), 3.74 (d, J=7.3 Hz, 1H), 3.68 (h, J=7.3, 6.9 Hz, 1H), 3.64-3.55 (m, 1H), 2.41-2.21 (m, 2H), 2.07 (dt, J=13.1, 6.8 Hz, 1H), 0.61-0.52 (m, 2H), 0.49-0.39 (m, 2H).

### Example 8: Synthesis of Compound 214

### Synthesis of Intermediate B]7

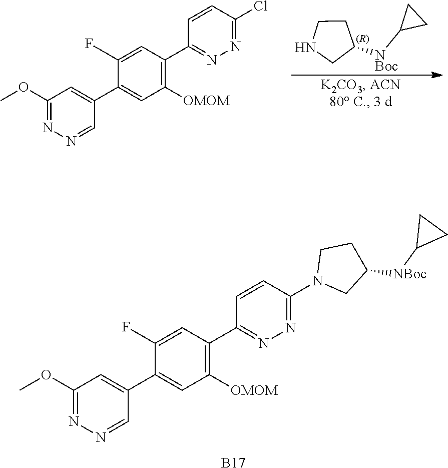

To a stirred solution of 3-chloro-6-[5-fluoro-2-(methoxymethoxy)-4-(6-methoxypyridazin-4-yl)phenyl]pyridazine (80 mg, 0.212 mmol, 1 equiv) and tert-butyl N-cyclopropyl-N-[(3S)-pyrrolidin-3-yl]carbamate (57.67 mg, 0.254 mmol, 1.2 equiv) in acetonitrile (1.6 mL) was added K₂CO₃ (88.04 mg, 0.636 mmol, 3 equiv) at room temperature. The resulting mixture was stirred for 2 days at 80° C., then concentrated under reduced pressure to give a residue. The residue was purified by silica gel column chromatography, eluted with CH₂Cl₂/MeOH (10:1) to afford tert-butyl N-cyclopropyl-N-[(3S)-1-{6-[5-fluoro-2-(methoxymethoxy)-4-(6-methoxypyridazin-4-yl)phenyl]pyridazin-3-yl}pyrrolidin-3-yl]carbamate (60 mg) as a solid. LCMS (ES, m/z): 567 [M+H]⁺.

### Synthesis of Compound 214

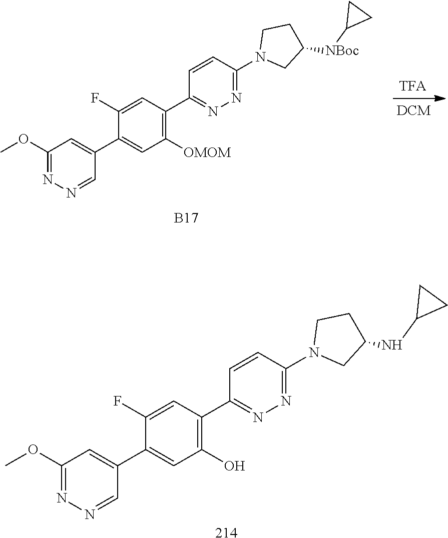

To a stirred solution of tert-butyl N-cyclopropyl-N-[(3S)-1-{6-[5-fluoro-2-(methoxymethoxy)-4-(6-methoxypyridazin-4-yl)phenyl]pyridazin-3-yl}pyrrolidin-3-yl]carbamate (60 mg, 0.106 mmol, 1 equiv) in DCM (1 mL) was added TFA (1 mL) at room temperature. The resulting mixture was stirred for 1 h at room temperature, then concentrated under reduced pressure to give a residue. The residue was purified by Prep-HPLC (Condition 5, Gradient 2) to afford 2-{6-[(3S)-3-(cyclopropylamino)pyrrolidin-1-yl]pyridazin-3-yl}-4-fluoro-5-(6-methoxypyridazin-4-yl)phenol (15 mg) as a solid. LCMS (ES, m/z): 423 [M+H]⁺. ¹H NMR (400 MHz, Methanol-d₄) δ 9.10 (d, J=1.8 Hz, 1H), 8.15 (d, J=9.8 Hz, 1H), 7.77 (d, J=12.3 Hz, 1H), 7.42 (s, 1H), 7.25 (d, J=6.7 Hz, 1H), 7.19 (d, J=9.7 Hz, 1H), 4.17 (s, 3H), 3.85 (dd, J=10.7, 6.3 Hz, 1H), 3.75 (s, 1H), 3.66 (q, J=7.5, 6.7 Hz, 1H), 3.64-3.55 (m, 1H), 3.50 (s, 1H), 2.35 (dq, J=13.0, 6.1 Hz, 1H), 2.26 (tt, J=7.0, 3.7 Hz, 1H), 2.07 (dq, J=13.6, 7.0 Hz, 1H), 0.61-0.52 (m, 2H), 0.44 (q, J=3.4, 2.8 Hz, 2H).

### Example 9: Synthesis of Compound 215

### Synthesis of Intermediate B18

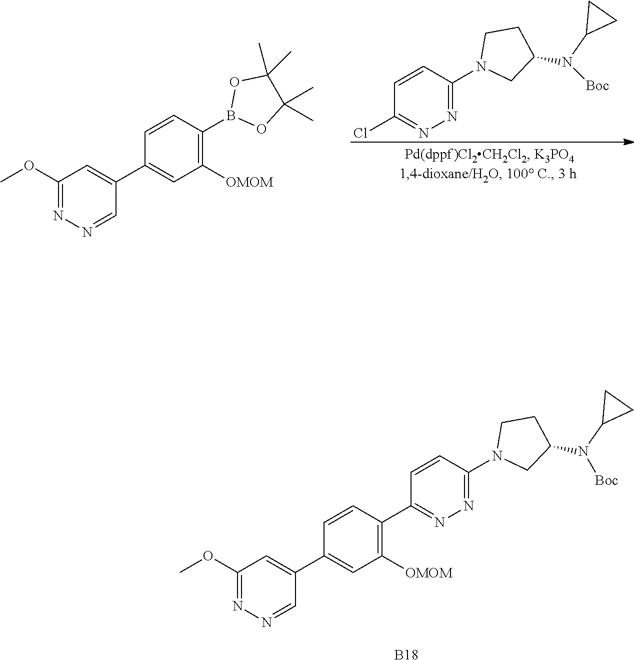

To a stirred mixture of 3-methoxy-5-[3-(methoxymethoxy)-4-(4,4,5,5-tetramethyl-1,3,2-dioxaborolan-2-yl)phenyl]pyridazine (150 mg, 0.403 mmol, 1 equiv) and tert-butyl N-[(3S)-1-(6-chloropyridazin-3-yl)pyrrolidin-3-yl]-N-cyclopropylcarbamate (163.85 mg, 0.484 mmol, 1.2 equiv) in 1,4-dioxane (2.5 mL) and H₂O (0.5 mL) was added K₃PO₄ (256.61 mg, 1.209 mmol, 3 equiv) and Pd(dppf)Cl₂·CH₂Cl₂ (32.83 mg, 0.040 mmol, 0.1 equiv) at room temperature under nitrogen atmosphere. The resulting mixture was stirred for 3 h at 100° C. under nitrogen atmosphere, then concentrated under reduced pressure to give a residue. The residue was purified by silica gel column chromatography, eluted with PE/EA (1:1) to afford tert-butyl N-cyclopropyl-N-[(3S)-1-{6-[2-(methoxymethoxy)-4-(6-methoxypyridazin-4-yl)phenyl]pyridazin-3-yl}pyrrolidin-3-yl]carbamate (70 mg, 32%) as a solid. LCMS (ES, m/z): 549[M+H]⁺.

### Synthesis of Compound 215

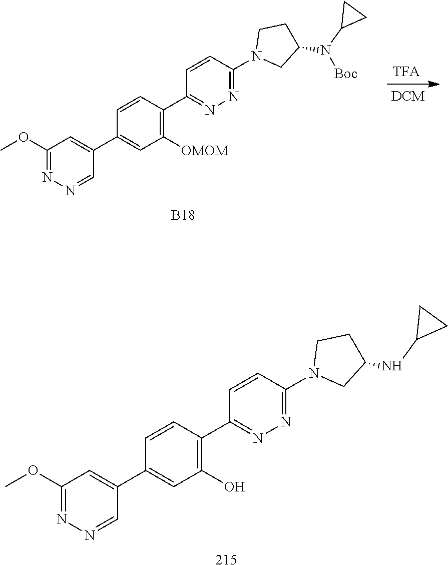

To a stirred mixture of tert-butyl N-cyclopropyl-N-[(3S)-1-{6-[2-(methoxymethoxy)-4-(6-methoxypyridazin-4-yl)phenyl]pyridazin-3-yl}pyrrolidin-3-yl]carbamate (70 mg, 0.128 mmol, 1 equiv) in DCM (1 mL) was added TFA (1 mL) at room temperature. The resulting mixture was stirred for 1 h at room temperature, then concentrated under reduced pressure to give a residue.

The residue was purified by Prep-HPLC (Condition 3, Gradient 2), followed by chiral HPLC (Condition 4, Gradient 1) to afford 2-{6-[(3S)-3-(cyclopropylamino)pyrrolidin-1-yl]pyridazin-3-yl}-5-(6-methoxypyridazin-4-yl)phenol (14.7 mg, 28%) as a solid. LCMS (ES, m/z): 405[M+H]. H NMR (300 MHz, Methanol-d₄) δ 9.20 (d, J=1.9 Hz, 1H), 8.19 (d, J=9.9 Hz, 1H), 7.97 (d, J=8.2 Hz, 1H), 7.47 (d, J=1.9 Hz, 1H), 7.41 (d, J=8.9 Hz, 2H), 7.21 (d, J=9.8 Hz, 1H), 4.17 (s, 3H), 3.86 (dd, J=10.8, 6.2 Hz, 1H), 3.75-3.59 (m, 3H), 3.49 (dd, J=10.8, 5.3 Hz, 1H), 2.41-2.20 (m, 2H), 2.07 (dd, J=12.4, 7.0 Hz, 1H), 0.61-0.53 (m, 2H), 0.49-0.38 (m, 2H).

### Example 10: Synthesis of Compound 216

### Synthesis of Intermediate B19

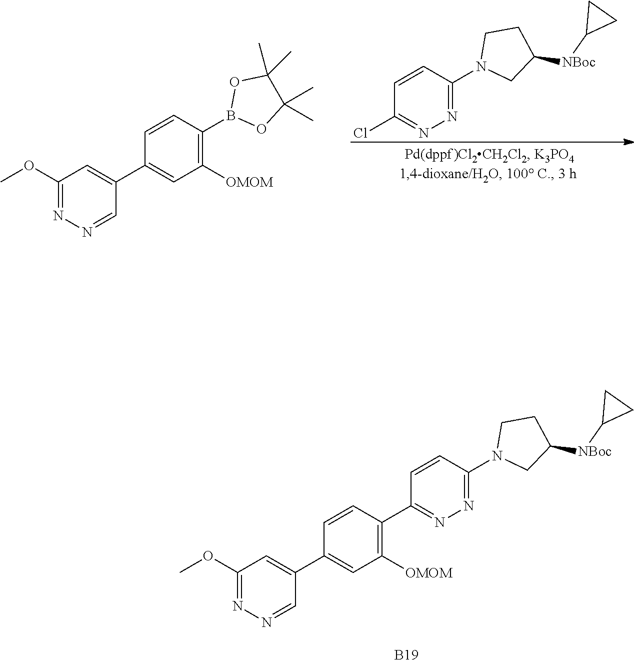

To a stirred mixture of 3-methoxy-5-[3-(methoxymethoxy)-4-(4,4,5,5-tetramethyl-1,3,2-dioxaborolan-2-yl)phenyl]pyridazine (150 mg, 0.403 mmol, 1 equiv) and tert-butyl N-[(3R)-1-(6-chloropyridazin-3-yl)pyrrolidin-3-yl]-N-cyclopropylcarbamate (163.85 mg, 0.484 mmol, 1.2 equiv) in 1,4-dioxane (2.5 mL) and H₂O (0.5 mL) was added K₃PO₄ (256.61 mg, 1.209 mmol, 3 equiv) and Pd(dppf)Cl₂—CH₂Cl₂ (32.83 mg, 0.040 mmol, 0.1 equiv) at room temperature under nitrogen atmosphere. The resulting mixture was stirred for 3 h at 100° C. under nitrogen atmosphere, then concentrated under reduced pressure to give a residue. The residue was purified by silica gel column chromatography, eluted with PE/EA (1:1) to afford tert-butyl N-cyclopropyl-N-[(3R)-1-{6-[2-(methoxymethoxy)-4-(6-methoxypyridazin-4-yl)phenyl]pyridazin-3-yl}pyrrolidin-3-yl]carbamate (70 mg, 32%) as a solid. LCMS (ES, m/z): 549[M+H]⁺.

### Synthesis of Compound 216

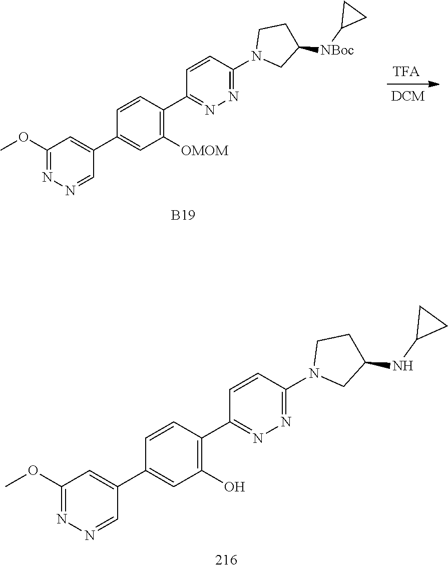

To a stirred solution of tert-butyl N-cyclopropyl-N-[(3R)-1-{6-[2-(methoxymethoxy)-4-(6-methoxypyridazin-4-yl)phenyl]pyridazin-3-yl}pyrrolidin-3-yl]carbamate (70 mg, 0.128 mmol, 1 equiv) in DCM (1 mL) was added TFA (1 mL) at room temperature. The resulting mixture was stirred for 1 h at room temperature, then concentrated under reduced pressure to give a residue. The residue was purified by Prep-HPLC (Condition 3, Gradient 2), followed by chiral HPLC (Condition 4, Gradient 1) to afford 2-{6-[(3R)-3-(cyclopropylamino)pyrrolidin-1-yl]pyridazin-3-yl}-5-(6-methoxypyridazin-4-yl)phenol (22.4 mg, 43%) as a solid. LCMS (ES, m/z): 405[M+H]. H NMR (300 MHz, Methanol-d₄) δ 9.20 (d, J=1.8 Hz, 1H), 8.19 (d, J=9.9 Hz, 1H), 7.96 (d, J=8.1 Hz, 1H), 7.47 (d, J=1.9 Hz, 1H), 7.40 (d, J=9.6 Hz, 2H), 7.21 (d, J=9.8 Hz, 1H), 4.17 (s, 3H), 3.88-3.56 (m, 4H), 3.55-3.44 (m, 1H), 2.39-2.20 (m, 2H), 2.13-2.01 (m, 1H), 0.57 (d, J=6.6 Hz, 2H), 0.44 (s, 2H).

### Example 11: Synthesis of Compound 217

### Synthesis of Intermediate B20

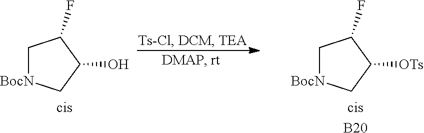 [SMILES: CC(C)(C)OC(=O)N1C[C@@H](O)[C@@H](F)C1 ; "B20" ; Cc1ccc(S(=O)(=O)O[C@@H]2CN(C(=O)OC(C)(C)C)C[C@@H]2F)cc1]

A mixture of tert-butyl (3S,4R)-3-fluoro-4-hydroxypyrrolidine-1-carboxylate (3.0 g, 14.618 mmol, 1.0 equiv), DCM (30 mL), TEA (2.96 g, 29.236 mmol, 2.0 equiv), TsCl (4.18 g, 21.927 mmol, 1.5 equiv) and DMAP (0.13 g, 1.462 mmol, 0.1 equiv) was stirred for 4 h at room temperature, then concentrated under reduced pressure to give a residue. The residue was purified by silica gel column chromatography, eluted with CH₂Cl₂/ EA (1:1) to afford tert-butyl (3S,4R)-3-fluoro-4-[(4-methylbenzenesulfonyl)oxy]pyrrolidine-1-carboxylate (5.0 g, 95%) as a solid. LCMS (ES, m/z): 360 [M+H]⁺.

### Synthesis of Intermediate B21

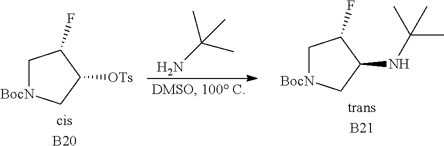

A mixture of tert-butyl (3S,4R)-3-fluoro-4-[(4-methylbenzenesulfonyl)oxy]pyrrolidine-1-carboxylate (2.0 g, 5.565 mmol, 1.0 equiv), DMSO (10 mL), and erbumine (4.07 g, 55.650 mmol, 10.0 equiv) was stirred for 3 days at 100° C. The resulting mixture was diluted with water (20 mL) and extracted with MTBE (3×30 mL). The organic layers were combined, then concentrated under reduced pressure to give a residue. The residue was purified by silica gel column chromatography, eluted with CH₂Cl₂/MeOH (10:1) to afford tert-butyl (3S,4S)-3-(tert-butylamino)-4-fluoropyrrolidine-1-carboxylate (0.3 g, 21%) as an oil. LCMS (ES, m/z): 261 [M+H]⁺.

### Synthesis of Intermediate B22

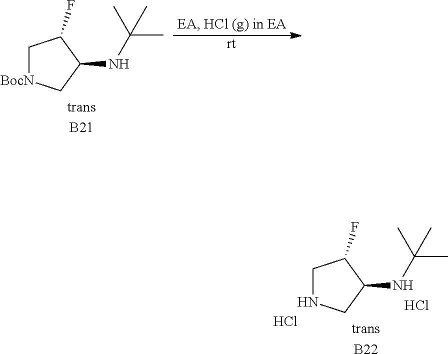 [SMILES: "B21" ; CC(C)(C)N[C@H]1CN(C(=O)OC(C)(C)C)C[C@@H]1F ; "B22" ; Cl ; CC(C)(C)N[C@H]1CNC[C@@H]1F ; Cl]

A mixture of tert-butyl (3 S,4S)-3-(tert-butylamino)-4-fluoropyrrolidine-1-carboxylate (0.3 g, 1.152 mmol, 1 equiv), ethyl acetate (2 mL) and HCl (gas) in ethyl acetate (2 mL) at room temperature. The resulting mixture was stirred for 4 h at room temperature, then concentrated under reduced pressure to afford (3S,4S)—N-tert-butyl-4-fluoropyrrolidin-3-amine dihydrochloride (0.2 g, 74%) as a solid. LCMS (ES, m/z): 161 [M+H]⁺.

### Synthesis of Intermediate B23

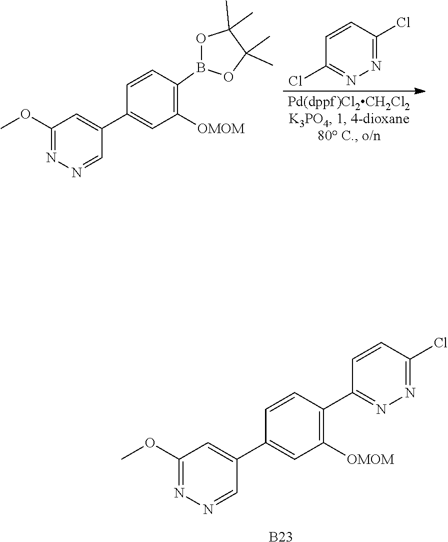

To a stirred mixture of 3-methoxy-5-[3-(methoxymethoxy)-4-(4,4,5,5-tetramethyl-1,3,2-dioxaborolan-2-yl)phenyl]pyridazine (1.2 g, 3.224 mmol, 1 equiv) and 3,6-dichloropyridazine (0.62 g, 4.191 mmol, 1.3 equiv) in 1,4-dioxane (24 mL) was added K₃PO₄ (1.37 g, 6.448 mmol, 2 equiv) and Pd(dppf)Cl₂. (0.26 g, 0.322 mmol, 0.1 equiv) at room temperature under nitrogen atmosphere. The resulting mixture was stirred overnight at 80° C. under nitrogen atmosphere, then concentrated under reduced pressure to give a residue. The residue was purified by silica gel column chromatography, eluted with CH₂Cl₂/MeOH (20:1) to afford 3-chloro-6-[2-(methoxymethoxy)-4-(6-methoxypyridazin-4-yl)phenyl]pyridazine (400 mg, 35%) as a solid. LCMS (ES, m/z): 359 [M+H]⁺.

### Synthesis of Intermediate B24

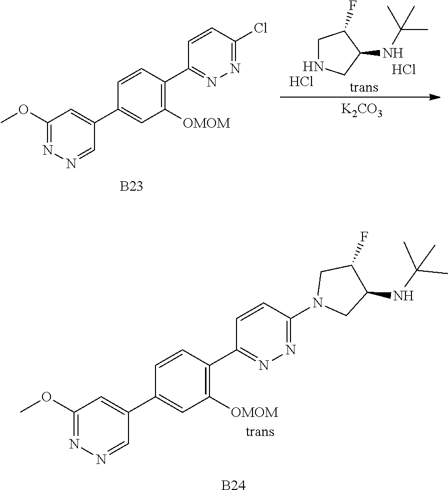

A mixture of 3-chloro-6-[2-(methoxymethoxy)-4-(6-methoxypyridazin-4-yl)phenyl]pyridazine (100 mg, 0.279 mmol, 1.0 equiv), DMSO (2 mL), (3S,4S)—N-tert-butyl-4-fluoropyrrolidin-3-amine (66.99 mg, 0.419 mmol, 1.5 equiv), and K₂CO₃ (115.56 mg, 0.837 mmol, 3.0 equiv) was stirred overnight at 100° C. The residue product was purified by reverse phase flash (Condition 1, Gradient 1) to afford (3S,4S)—N-tert-butyl-4-fluoro-1-{6-[2-(methoxymethoxy)-4-(6-methoxypyridazin-4-yl)phenyl]pyridazin-3-yl}pyrrolidin-3-amine (20 mg, 15%) as a solid.

LCMS (ES, m/z): 483 [M+H]⁺.

### Synthesis of Compound 217

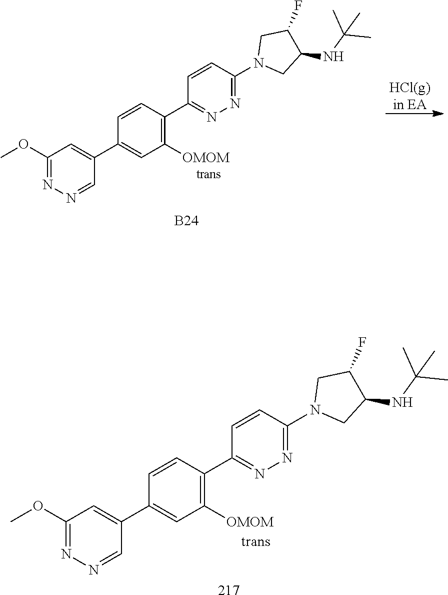

A mixture of(3S,4S)—N-tert-butyl-4-fluoro-1-{6-[2-(methoxymethoxy)-4-(6-methoxypyridazin-4-yl)phenyl]pyridazin-3-yl}pyrrolidin-3-amine (20 mg, 0.041 mmol, 1 equiv), ethyl acetate (1 mL), and HCl (gas) in ethyl acetate (1 mL) was stirred for 3 h at room temperature. The mixture was purified by reverse phase flash (Condition 1, Gradient 1), followed by chiral SFC (Condition 5, Gradient 1) to afford 2-{6-[(3S,4R)-3-(tert-butylamino)-4-fluoropyrrolidin-1-yl]pyridazin-3-yl}-5-(6-methoxypyridazin-4-yl)phenol (4 mg) as a solid. LCMS (ES, m/z): 439 [M+H]⁺. ¹H NMR (400 MHz, Chloroform-d) δ 13.78 (s, 1H), 9.16-9.11 (m, 1H), 7.89 (d, J=9.6 Hz, 1H), 7.71 (d, J=8.3 Hz, 1H), 7.38-7.33 (m, 1H), 7.19 (d, J=8.2 Hz, 1H), 7.16-7.11 (m, 1H), 6.90 (d, J=9.7 Hz, 1H), 5.09 (s, 1H), 4.19 (s, 3H), 3.98 (q, J=5.7, 4.7 Hz, 2H), 3.82 (dt, J=36.7, 12.8 Hz, 1H), 3.73-3.65 (m, 1H), 3.50 (d, J=10.9 Hz, 1H), 1.16 (s, 9H).

### Example 12: Synthesis of Compound 218

### Synthesis of Intermediate B25

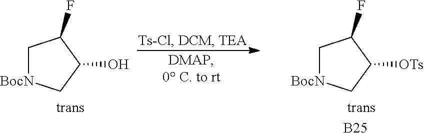 [SMILES: CC(C)(C)OC(=O)N1C[C@@H](O)[C@H](F)C1 ; "B25" ; Cc1ccc(S(=O)(=O)O[C@@H]2CN(C(=O)OC(C)(C)C)C[C@H]2F)cc1]

A mixture of tert-butyl (3R,4R)-3-fluoro-4-hydroxypyrrolidine-1-carboxylate (4.0 g, 19.490 mmol, 1.0 equiv), DCM (40 mL), TEA (3.94 g, 38.980 mmol, 2.0 equiv), TsCl (5.57 g, 29.235 mmol, 1.5 equiv) and DMAP (0.24 g, 1.949 mmol, 0.1 equiv) was stirred for 6 h at room temperature. The resulting mixture was concentrated under reduced pressure to give a residue. The residue was purified by silica gel column chromatography, eluted with CH₂Cl₂/ EA (1:1) to afford tert-butyl (3R,4R)-3-fluoro-4-[(4-methylbenzenesulfonyl)oxy]pyrrolidine-1-carboxylate (6.0 g, 86%) as a solid. LCMS (ES, m/z): 360 [M+H]⁺.

### Synthesis of Intermediate B26

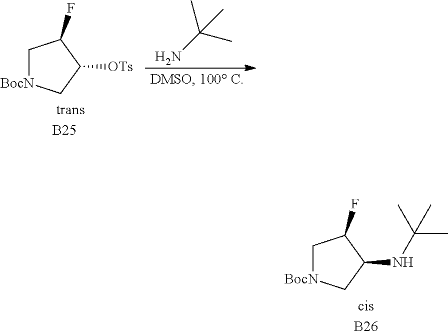

A mixture of tert-butyl (3R,4R)-3-fluoro-4-[(4-methylbenzenesulfonyl)oxy]pyrrolidine-1-carboxylate (2.0 g, 5.565 mmol, 1 equiv), DMSO (10 mL), and erbumine (4.07 g, 55.650 mmol, 10 equiv) at room temperature. The resulting mixture was stirred for 3 days at 100° C. The resulting mixture was diluted with water (20 mL) and extracted with MTBE (3×30 mL). The organic layers were combined and concentrated under reduced pressure to give a residue. The residue was purified by silica gel column chromatography, eluted with CH₂Cl₂/MeOH (10:1) to afford tert-butyl (3S,4R)-3-(tert-butylamino)-4-fluoropyrrolidine-1-carboxylate (0.3 g, 21%) as an oil. LCMS (ES, m/z): 261 [M+H]⁺.

### Synthesis Intermediate B27

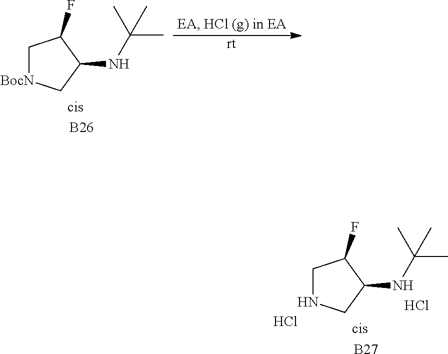 [SMILES: "B26" ; CC(C)(C)N[C@H]1CN(C(=O)OC(C)(C)C)C[C@H]1F ; "B27" ; Cl ; CC(C)(C)N[C@H]1CNC[C@H]1F ; Cl]

A mixture of tert-butyl (3S,4R)-3-(tert-butylamino)-4-fluoropyrrolidine-1-carboxylate (0.3 g, 1.152 mmol, 1 equiv), ethyl acetate (2 mL), and HCl (gas) in ethyl acetate (2 mL) at room temperature. The resulting mixture was stirred for 4 h at room temperature, then concentrated under reduced pressure to afford (3S,4R)—N-tert-butyl-4-fluoropyrrolidin-3-amine dihydrochloride (0.2 g, 75%) as a solid. LCMS (ES, m/z): 161 [M+H]⁺.

### Synthesis of Intermediate B28

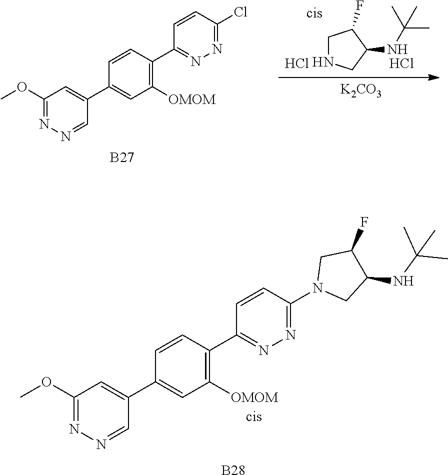

A mixture of 3-chloro-6-[2-(methoxymethoxy)-4-(6-methoxypyridazin-4-yl)phenyl]pyridazine (100 mg, 0.279 mmol, 1.0 equiv), DMSO (1 mL), (3S,4R)—N-tert-butyl-4-fluoropyrrolidin-3-amine (89.32 mg, 0.558 mmol, 2.0 equiv), and K₂CO₃ (116.41 mg, 0.837 mmol, 3.0 equiv) was stirred for 16 h at 100° C. The residue was purified by reverse phase flash (Condition 1, Gradient 1) to afford (3S,4R)—N-tert-butyl-4-fluoro-1-{6-[2-(methoxymethoxy)-4-(6-methoxypyridazin-4-yl)phenyl]pyridazin-3-yl}pyrrolidin-3-amine (20 mg, 15%) as a solid. LCMS (ES, m/z): 483 [M+H]⁺.

### Synthesis of Compound 218

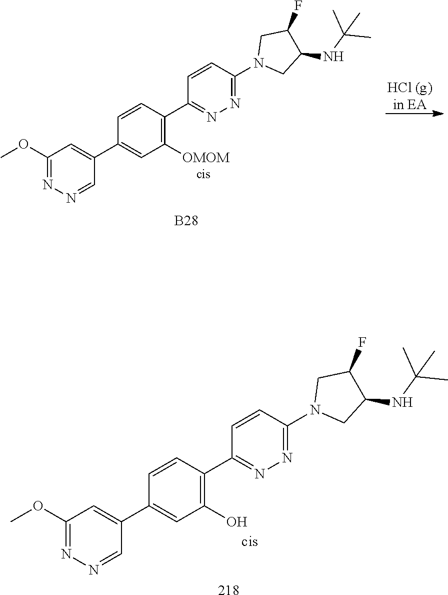

A mixture of(3S,4R)—N-tert-butyl-4-fluoro-1-{6-[2-(methoxymethoxy)-4-(6-methoxypyridazin-4-yl)phenyl]pyridazin-3-yl}pyrrolidin-3-amine (20 mg, 0.041 mmol, 1 equiv), ethyl acetate (1 mL), and HCl (gas) in ethyl acetate (1 mL) was stirred for 3 h at room temperature. The resulting mixture was purified by reverse phase flash (Condition 1, Gradient 1), followed by chiral SFC (Condition 5, Gradient 1) to afford 2-{6-[(3S,4R)-3-(tert-butylamino)-4-fluoropyrrolidin-1-yl]pyridazin-3-yl}-5-(6-methoxypyridazin-4-yl)phenol (4 mg) as a solid. LCMS (ES, m/z): 439 [M+H]⁺. ¹H NMR (400 MHz, Chloroform-d) δ 13.77 (s, 1H), 9.14 (d, J=1.9 Hz, 1H), 7.90 (d, J=9.8 Hz, 1H), 7.71 (d, J=8.3 Hz, 1H), 7.36 (d, J=1.9 Hz, 1H), 7.20 (dd, J=8.3, 1.9 Hz, 1H), 7.15 (d, J=1.9 Hz, 1H), 6.91 (d, J=9.7 Hz, 1H), 5.33 (s, 1H), 4.21 (s, 3H), 4.08 (dd, J=25.2, 13.8 Hz, 1H), 3.99-3.91 (m, 1H), 3.89-3.81 (m, 1H), 3.75 (s, 1H), 3.68 (s, 1H), 3.40 (s, 1H), 1.29 (s, 9H).

### Example 13: Synthesis of Compound 219

### Synthesis of Intermediate B29

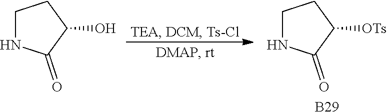 [SMILES: O=C1NCC[C@@H]1O ; "B29" ; Cc1ccc(S(=O)(=O)O[C@H]2CCNC2=O)cc1]

A mixture of (3S)-3-hydroxypyrrolidin-2-one (5.0 g, 49.454 mmol, 1.0 equiv), DCM (50 mL), TEA (10.01 g, 98.908 mmol, 2.0 equiv), TsCl (14.14 g, 74.181 mmol, 1.5 equiv), and DMAP (0.60 g, 4.945 mmol, 0.1 equiv) was stirred overnight at room temperature. The resulting mixture was concentrated under reduced pressure to give a residue. The residue was purified by silica gel column chromatography, eluted with CH₂Cl₂/ EA (1:1) to afford (3S)-2-oxopyrrolidin-3-yl 4-methylbenzenesulfonate (12.0 g, 95%) as a solid. LCMS (ES, m/z): 256 [M+H]⁺.

### Synthesis of Intermediate B30

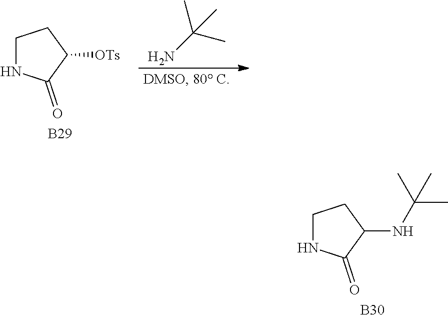

A mixture of (3S)-2-oxopyrrolidin-3-yl 4-methylbenzenesulfonate (5.0 g, 19.586 mmol, 1 equiv), DMSO (25 mL), and erbumine (14.32 g, 195.860 mmol, 10.0 equiv) at room temperature. The resulting mixture was stirred overnight at 80° C., then concentrated under reduced pressure to give a residue. The residue was purified by reverse phase flash (Condition 1, Gradient 1) to afford 3-(tert-butylamino)pyrrolidin-2-one (1.5 g, 49%) as a solid. LCMS (ES, m/z): 157 [M+H]⁺.

### Synthesis of Intermediate B31

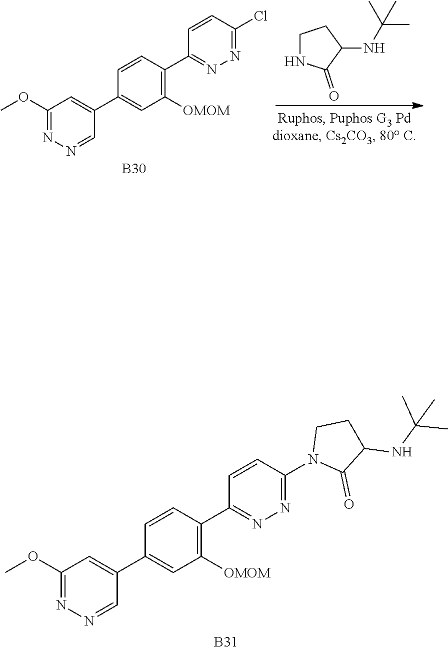

A mixture of 3-chloro-6-[2-(methoxymethoxy)-4-(6-methoxypyridazin-4-yl)phenyl]pyridazine (60 mg, 0.167 mmol, 1.0 equiv), dioxane (1 mL), 3-(tert-butylamino)pyrrolidin-2-one (31.35 mg, 0.200 mmol, 1.2 equiv), Cs₂CO₃ (163.46 mg, 0.501 mmol, 3.0 equiv), RuPhos (15.61 mg, 0.033 mmol, 0.2 equiv) and 3rd Generation RuPhos precatalyst (13.99 mg, 0.017 mmol, 0.1 equiv) was stirred overnight at 80° C. under nitrogen atmosphere. The resulting mixture was concentrated under reduced pressure to give a residue. The residue was purified by silica gel column chromatography, eluted with CH₂Cl₂/MeOH (20:1) to afford 3-(tert-butylamino)-1-{6-[2-(methoxymethoxy)-4-(6-methoxypyridazin-4-yl)phenyl]pyridazin-3-yl}pyrrolidin-2-one (40 mg, 50%) as a solid. LCMS (ES, m/z): 479 [M+H]⁺.

### Synthesis of Compound 219

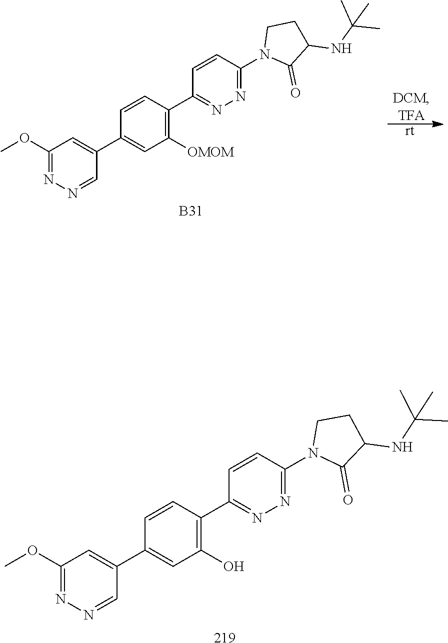

A mixture of 3-(tert-butylamino)-1-{6-[2-(methoxymethoxy)-4-(6-methoxypyridazin-4-yl)phenyl]pyridazin-3-yl}pyrrolidin-2-one (40 mg, 0.084 mmol, 1 equiv), DCM (1 mL) and TFA (1 mL) was stirred for 3 h at room temperature. The reaction mixture was concentrated under reduced pressure to give a residue. The residue was triturated with MTBE to afford 3-(tert-butylamino)-1-{6-[2-hydroxy-4-(6-methoxypyridazin-4-yl)phenyl]pyridazin-3-yl}pyrrolidin-2-one (12 mg, 26%) as a solid. LCMS (ES, m/z): 435 [M+H]⁺. ¹H NMR (400 MHz, DMSO-d₆) δ 12.54 (s, 1H), 9.35 (d, J=1.9 Hz, 1H), 9.09 (s, 1H), 8.87 (d, J=12.2 Hz, 1H), 8.70 (d, J=9.6 Hz, 1H), 8.65 (d, J=9.7 Hz, 1H), 8.13 (d, J=8.1 Hz, 1H), 7.60-7.53 (m, 3H), 4.66 (s, 1H), 4.44 (t, J=9.8 Hz, 1H), 4.11 (s, 3H), 4.00 (td, J=10.5, 6.4 Hz, 1H), 2.82-2.75 (m, 2H), 1.41 (s, 9H).

### Example 14: Synthesis of Compound 161

### Synthesis of Intermediate B32

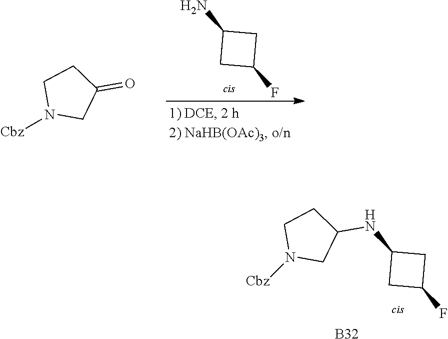 [SMILES: O=C1CCN(C(=O)OCc2ccccc2)C1 ; N[C@H]1C[C@@H](F)C1 ; "B32" ; O=C(OCc1ccccc1)N1CCC(N[C@H]2C[C@@H](F)C2)C1]

To a stirred solution of benzyl 3-oxopyrrolidine-1-carboxylate (3 g, 13.684 mmol, 1 equiv) in DCE (60 mL) was added (cis)-3-fluorocyclobutan-1-amine hydrochloride (2.06 g, 16.421 mmol, 1.2 equiv) at room temperature. The resulting mixture was stirred for 2 h at room temperature. To the reaction mixture was added STAB (8.70 g, 41.052 mmol, 3 equiv) in portions at room temperature. The resulting mixture was stirred for 4 h at room temperature, then concentrated under vacuum to give a residue. The residue was purified by silica gel column chromatography, eluted with CH₂Cl₂/MeOH (10:1) to afford benzyl (3R)-3-{[(1s,3s)-3-fluorocyclobutyl]amino}pyrrolidine-1-carboxylate (2.1 g, 52%) as an oil. LCMS (ES, m/z): 293 [M+H]⁺.

### Synthesis of Intermediate B33

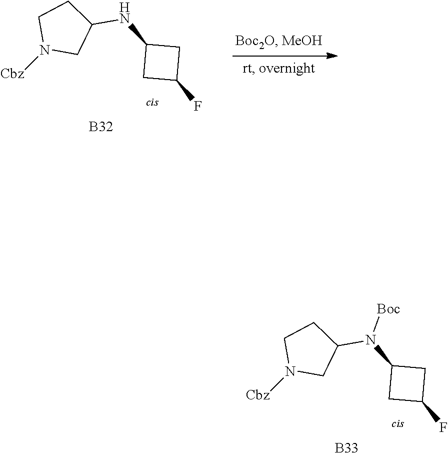 [SMILES: "B32" ; O=C(OCc1ccccc1)N1CCC(N[C@H]2C[C@@H](F)C2)C1 ; "B33" ; CC(C)(C)OC(=O)N(C1CCN(C(=O)OCc2ccccc2)C1)[C@H]1C[C@@H](F)C1]

To a stirred solution of benzyl (3R)-3-{[(1s,3s)-3-fluorocyclobutyl]amino}pyrrolidine-1-carboxylate (2.1 g, 7.183 mmol, 1 equiv) in methanol (21 mL) was added Boc₂O (3.14 g, 14.366 mmol, 2 equiv) at room temperature. The resulting mixture was stirred overnight at room temperature, then concentrated under vacuum to give a residue. The residue was purified by silica gel column chromatography, eluted with PE/EA (1:1) to afford benzyl (3R)-3-[(tert-butoxycarbonyl)[(1s,3s)-3-fluorocyclobutyl]amino]pyrrolidine-1-carboxylate (2.4 g, 85%) as an oil. LCMS (ES, m/z): 393 [M+H]⁺.

### Synthesis of Intermediate B34

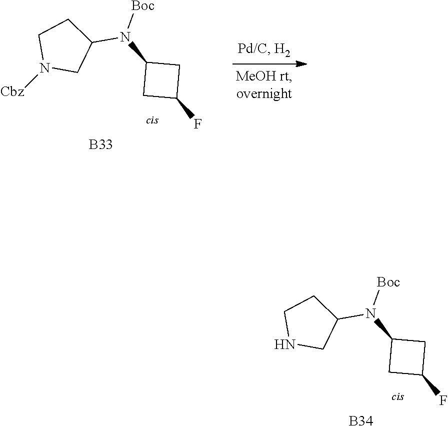 [SMILES: "B33" ; CC(C)(C)OC(=O)N(C1CCN(C(=O)OCc2ccccc2)C1)[C@H]1C[C@@H](F)C1 ; "B34" ; CC(C)(C)OC(=O)N(C1CCNC1)[C@H]1C[C@@H](F)C1]

To a solution of benzyl (3R)-3-[(tert-butoxycarbonyl)[(1s,3s)-3-fluorocyclobutyl]amino]pyrrolidine-1-carboxylate (2.4 g, 6.115 mmol, 1 equiv) in methanol (50 mL) was added Pd/C (10%, 500 mg) under nitrogen atmosphere. The resulting mixture was stirred at room temperature overnight under hydrogen atmosphere using a hydrogen balloon, filtered through a Celite pad and concentrated under reduced pressure to afford tert-butyl N-[(3R)-pyrrolidin-3-yl]-N-[(1s,3s)-3-fluorocyclobutyl]carbamate (1.2 g, 76%) as an oil. LCMS (ES, m/z): 259 [M+H]⁺.

### Synthesis of Intermediate B35

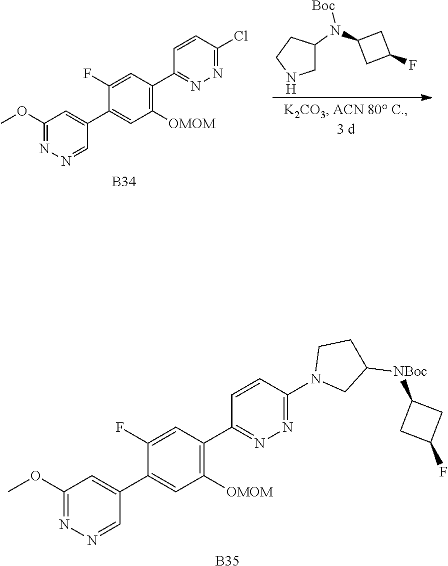

To a stirred mixture of 3-chloro-6-[5-fluoro-2-(methoxymethoxy)-4-(6-methoxypyridazin-4-yl)phenyl]pyridazine (80 mg, 0.212 mmol, 1 equiv) and tert-butyl N-(pyrrolidin-3-yl)-N-[(1s,3s)-3-fluorocyclobutyl]carbamate (65.82 mg, 0.254 mmol, 1.2 equiv) in acetonitrile (1.6 mL) was added K₂CO₃ (88.04 mg, 0.636 mmol, 3 equiv) at room temperature. The resulting mixture was stirred for 3 days at 80° C., then concentrated under reduced pressure to give a residue. The residue was purified by silica gel column chromatography, eluted with CH₂Cl₂/MeOH (10:1) to afford tert-butyl N-(1-{6-[5-fluoro-2-(methoxymethoxy)-4-(6-methoxypyridazin-4-yl)phenyl]pyridazin-3-yl}pyrrolidin-3-yl)-N-[(1s,3s)-3-fluorocyclobutyl]carbamate (60 mg, 47%) as a solid. LCMS (ES, m/z): 599 [M+H]⁺.

### Synthesis of Compound 161

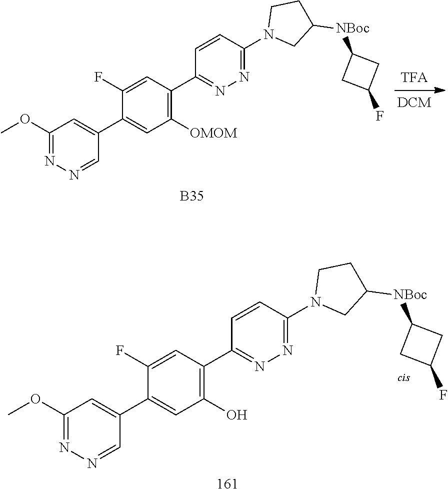

To a stirred solution of tert-butyl N-(1-{6-[5-fluoro-2-(methoxymethoxy)-4-(6-methoxypyridazin-4-yl)phenyl]pyridazin-3-yl}pyrrolidin-3-yl)-N-[(1s,3s)-3-fluorocyclobutyl]carbamate (60 mg, 0.100 mmol, 1 equiv) in DCM (1 mL) was added TFA (1 mL) at room temperature. The resulting mixture was stirred for 1 h at room temperature, then concentrated under reduced pressure to give a residue. The residue was purified by Prep-HPLC (Condition 4, Gradient 2) to afford 4-fluoro-5-(6-methoxypyridazin-4-yl)-2-[6-(3-{[(1s,3s)-3-fluorocyclobutyl]amino}pyrrolidin-1-yl)pyridazin-3-yl]phenol (39 mg, 57%) as a solid. LCMS (ES, m/z): 455 [M+H]⁺. ¹H NMR (300 MHz, DMSO-d₆) δ 13.58 (s, 1H), 9.17 (t, J=1.9 Hz, 1H), 8.40 (d, J=9.8 Hz, 1H), 8.04 (d, J=12.4 Hz, 1H), 7.48-7.39 (m, 1H), 7.37-7.27 (m, 2H), 5.40 (t, J=4.8 Hz, 1H), 5.22 (t, J=4.9 Hz, OH), 4.16 (t, J=7.5 Hz, 1H), 4.10 (s, 3H), 3.97 (s, 1H), 3.88 (dd, J=11.9, 6.1 Hz, 1H), 3.82-3.63 (m, 2H), 3.62 (d, J=7.6 Hz, 1H), 2.60 (dd, J=23.5, 9.2 Hz, 4H), 2.41 (dt, J=14.3, 6.9 Hz, 1H), 2.22 (dd, J=12.8, 6.7 Hz, 1H).

### Example 15: Synthesis of Compound 162

### Synthesis of Intermediate B36

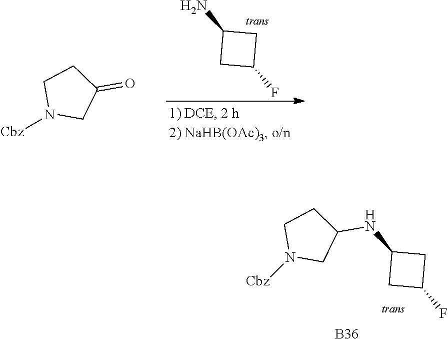 [SMILES: O=C1CCN(C(=O)OCc2ccccc2)C1 ; N[C@H]1C[C@H](F)C1 ; "B36" ; O=C(OCc1ccccc1)N1CCC(N[C@H]2C[C@H](F)C2)C1]

To a stirred solution of benzyl 3-oxopyrrolidine-1-carboxylate (3 g, 13.684 mmol, 1 equiv) in DCE (60 mL) were added (trans)-3-fluorocyclobutan-1-amine (1.34 g, 15.052 mmol, 1.1 equiv) at room temperature. The resulting mixture was stirred for 2 h at room temperature. To the above mixture was added STAB (8.70 g, 41.052 mmol, 3 equiv) in portions at room temperature. The resulting mixture was stirred for additional 4 h at room temperature. The resulting mixture was concentrated under vacuum. The residue was purified by silica gel column chromatography, eluted with CH₂Cl₂/MeOH (10:1) to afford benzyl-3-{[(1r,3r)-3-fluorocyclobutyl]amino}pyrrolidine-1-carboxylate (1.9 g, 47.49%) as an oil. LCMS (ES, m/z): 293 [M+H]⁺.

### Synthesis of Intermediate B37

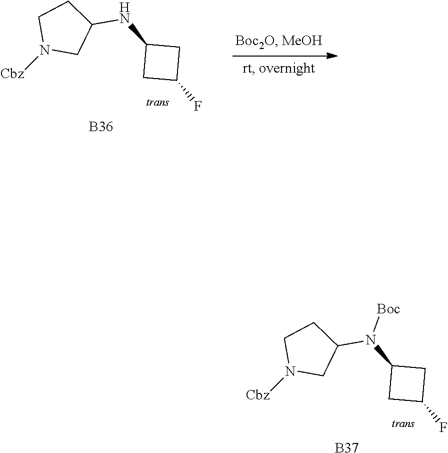 [SMILES: "B36" ; O=C(OCc1ccccc1)N1CCC(N[C@H]2C[C@H](F)C2)C1 ; "B37" ; CC(C)(C)OC(=O)N(C1CCN(C(=O)OCc2ccccc2)C1)[C@H]1C[C@H](F)C1]

To a stirred solution of benzyl-3-{[(1s,3s)-3-fluorocyclobutyl]amino}pyrrolidine-I-carboxylate (2.1 g, 7.183 mmol, 1 equiv) in methanol (21 mL) was added Boc₂O (3.14 g, 14.366 mmol, 2 equiv) at room temperature. The resulting mixture was stirred overnight at room temperature, then concentrated under vacuum to give a residue. The residue was purified by silica gel column chromatography, eluted with PE/EA (1:1) to afford benzyl-3-[(tert-butoxycarbonyl)[(1s,3s)-3-fluorocyclobutyl]amino]pyrrolidine-1-carboxylate (2.4 g, 85%) as an oil. LCMS (ES, m/z): 393 [M+H]⁺.

### Synthesis of Intermediate B38

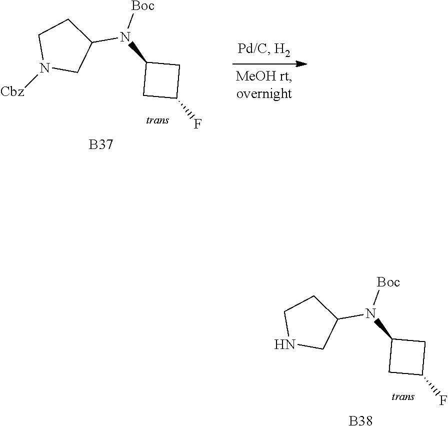 [SMILES: "B37" ; CC(C)(C)OC(=O)N(C1CCN(C(=O)OCc2ccccc2)C1)[C@H]1C[C@H](F)C1 ; "B38" ; CC(C)(C)OC(=O)N(C1CCNC1)[C@H]1C[C@H](F)C1]

To a solution of benzyl-3-[(tert-butoxycarbonyl)[(1r,3r)-3-fluorocyclobutyl]amino]pyrrolidine-1-carboxylate (1.8 g, 4.586 mmol, 1 equiv) in methanol (50 mL) was added Pd/C (10%, 400 mg) under nitrogen atmosphere. The mixture was stirred at room temperature overnight under hydrogen atmosphere using a hydrogen balloon, filtered through a Celite pad, and the filtrate concentrated under reduced pressure to afford tert-butyl N-(pyrrolidin-3-yl)-N-[(1r,3r)-3-fluorocyclobutyl]carbamate (1.0 g, 84%) as an oil. LCMS (ES, m/z): 259 [M+H]⁺.

### Synthesis of Intermediate B39

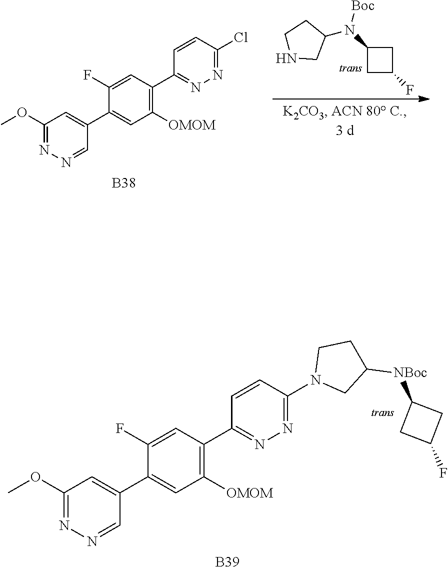

To a stirred mixture of 3-chloro-6-[5-fluoro-2-(methoxymethoxy)-4-(6-methoxypyridazin-4-yl)phenyl]pyridazine (80 mg, 0.212 mmol, 1 equiv) and tert-butyl N-(pyrrolidin-3-yl)-N-[(1r,3r)-3-fluorocyclobutyl]carbamate (65.82 mg, 0.254 mmol, 1.2 equiv) in acetonitrile (1.6 mL) was added K₂CO₃ (88.04 mg, 0.636 mmol, 3 equiv) at room temperature. The resulting mixture was stirred for 3 days at 80° C., then concentrated under reduced pressure to give a residue. The residue was purified by silica gel column chromatography, eluted with CH₂Cl₂/MeOH (10:1) to afford tert-butyl N-(1-{6-[5-fluoro-2-(methoxymethoxy)-4-(6-methoxypyridazin-4-yl)phenyl]pyridazin-3-yl}pyrrolidin-3-yl)-N-[(1r,3r)-3-fluorocyclobutyl]carbamate (60 mg, 47%) as a solid. LCMS (ES, m/z): 599 [M+H]⁺.

### Synthesis of Compound 162

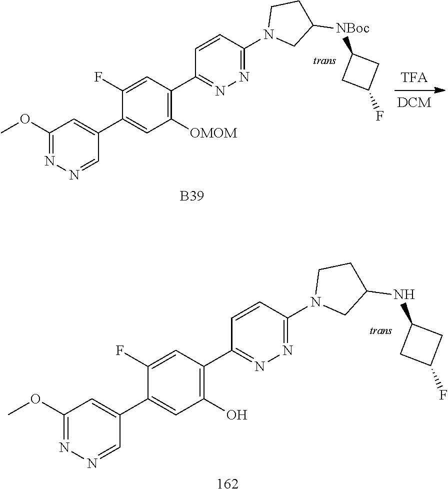

To a stirred solution of tert-butyl N-(1-{6-[5-fluoro-2-(methoxymethoxy)-4-(6-methoxypyridazin-4-yl)phenyl]pyridazin-3-yl}pyrrolidin-3-yl)-N-[(1r,3r)-3-fluorocyclobutyl]carbamate (60 mg, 0.100 mmol, 1 equiv) in DCM (1 mL) was added TFA (1 mL) at room temperature. The resulting mixture was stirred for 1 h at room temperature, then concentrated under reduced pressure to give a residue. The residue was purified by Prep-HPLC (Condition 4, Gradient 2) to afford 4-fluoro-5-(6-methoxypyridazin-4-yl)-2-[6-(3-{[(1r,3r)-3-fluorocyclobutyl]amino}pyrrolidin-1-yl)pyridazin-3-yl]phenol (40.7 mg, 60%) as a solid. LCMS (ES, m/z): 455 [M+H]⁺. ¹H NMR (300 MHz, DMSO-d₆) δ 13.55 (s, 1H), 9.17 (t, J=1.9 Hz, 1H), 8.39 (d, J=9.8 Hz, 1H), 8.04 (d, J=12.4 Hz, 1H), 7.45 (d, J=1.3 Hz, 1H), 7.37-7.27 (m, 2H), 4.94 (dt, J=55.9, 6.7 Hz, 1H), 4.10 (s, 3H), 4.02-3.82 (m, 2H), 3.73 (td, J=10.3, 9.0, 5.4 Hz, 2H), 3.67-3.54 (m, 1H), 3.48 (d, J=9.2 Hz, 1H), 2.89-2.75 (m, 2H), 2.42 (dd, J=14.0, 7.5 Hz, 3H), 2.27-2.16 (m, 1H).

### Example 16: Synthesis of Compound 220

### Synthesis of Intermediate B40

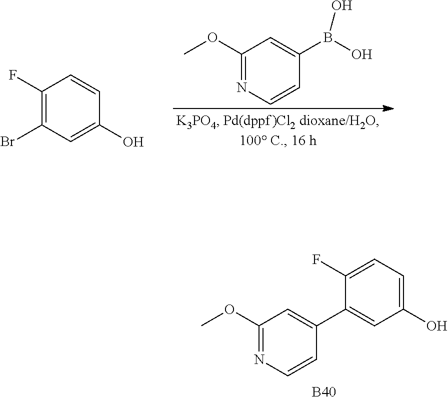 [SMILES: Oc1ccc(F)c(Br)c1 ; COc1cc(B(O)O)ccn1 ; "B40" ; COc1cc(-c2cc(O)ccc2F)ccn1]

To a stirred mixture of 3-bromo-4-fluorophenol (3 g, 15.707 mmol, 1 equiv) and 2-methoxypyridin-4-ylboronic acid (2.88 g, 18.848 mmol, 1.2 equiv) in a mixture of 1,4-dioxane and H₂O (50 mL/10 mL) was added K₃PO₄ (10.00 g, 47.121 mmol, 3 equiv) and Pd(dppf)Cl₂ (0.57 g, 0.785 mmol, 0.05 equiv) at room temperature under nitrogen atmosphere. The resulting mixture was stirred overnight at 100° C. under nitrogen atmosphere, then concentrated under vacuum to give a residue. The residue was purified by silica gel column chromatography, eluted with PE/EA (3:1) to afford 4-fluoro-3-(2-methoxypyridin-4-yl)phenol (1.8 g, 52%) as a solid.

LCMS (ES, m/z): 220 [M+H]⁺.

### Synthesis of Intermediate B41

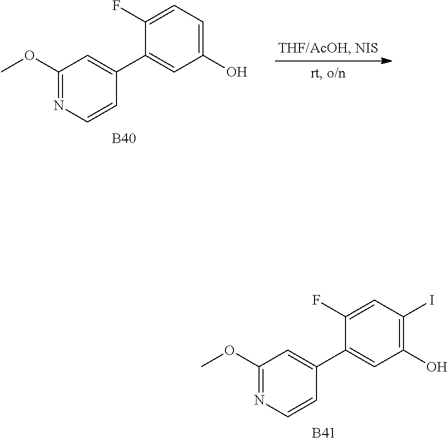 [SMILES: "B40" ; COc1cc(-c2cc(O)ccc2F)ccn1 ; "B41" ; COc1cc(-c2cc(O)c(I)cc2F)ccn1]

To a stirred mixture of 4-fluoro-3-(2-methoxypyridin-4-yl)phenol (1.8 g, 8.211 mmol, 1 equiv) in a mixture of THF and AcOH (40 mL/40 mL) was added NIS (1.85 g, 8.211 mmol, 1 equiv) in portions at room temperature. The resulting mixture was stirred overnight at room temperature, then concentrated under vacuum to give a residue. The residue was purified by Prep-HPLC (Condition 4, Gradient 2) to afford 4-fluoro-2-iodo-5-(2-methoxypyridin-4-yl)phenol (1.2 g, 42%) as a solid. LCMS (ES, m/z): 346 [M+H]⁺.

Synthesis of PH-Intermediate B42

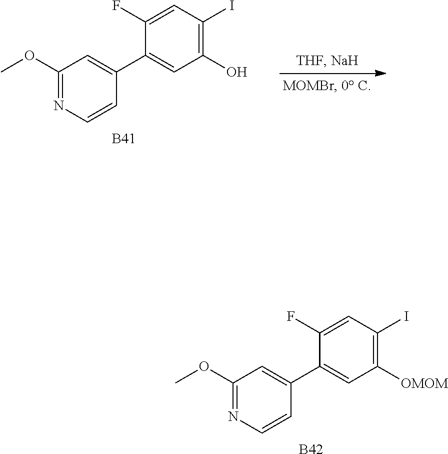

To a stirred solution of 4-fluoro-2-iodo-5-(2-methoxypyridin-4-yl)phenol (1.0 g, 2.898 mmol, 1.0 equiv) in THE (10 mL) was added NaH (0.08 g, 3.478 mmol, 1.2 equiv) in portions at 0° C. under nitrogen atmosphere, then stirred for 30 min at 0° C. under nitrogen atmosphere. To the resulting mixture was added bromo(methoxy)methane (0.33 g, 2.608 mmol, 0.9 equiv) dropwise at 0° C. The resulting mixture was stirred for an additional 30 min at 0° C., then quenched with methanol at 0° C. and concentrated under reduced pressure to give a residue. The residue was purified by silica gel column chromatography, eluted with PE/EA (5:1) to afford 4-[2-fluoro-4-iodo-5-(methoxymethoxy)phenyl]-2-methoxypyridine (0.6 g, 53%) as a solid. LCMS (ES, m/z): 390 [M+H]⁺.

### Synthesis of Intermediate B43

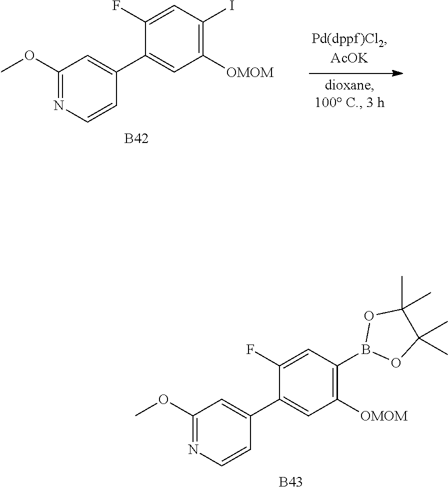

A mixture of 4-[2-fluoro-4-iodo-5-(methoxymethoxy)phenyl]-2-methoxypyridine (600 mg, 1.542 mmol, 1.0 equiv), dioxane (12 mL), 4,4,5,5-tetramethyl-2-(tetramethyl-1,3,2-dioxaborolan-2-yl)-1,3,2-dioxaborolane (1566.05 mg, 6.168 mmol, 4.0 equiv), AcOK (453.93 mg, 4.626 mmol, 3.0 equiv), and Pd(dppf)Cl₂ (112.81 mg, 0.154 mmol, 0.1 equiv) was stirred for 3 h at 100° C. under nitrogen atmosphere, then concentrated under reduced pressure to give a residue. The residue was purified by silica gel column chromatography, eluted with PE/EA (1:1) to afford 4-[2-fluoro-5-(methoxymethoxy)-4-(4,4,5,5-tetramethyl-1,3,2-dioxaborolan-2-yl)phenyl]-2-methoxypyridine (500 mg, 83%) as an oil. LCMS (ES, m/z): 390 [M+H]⁺.

### Synthesis of Intermediate B44

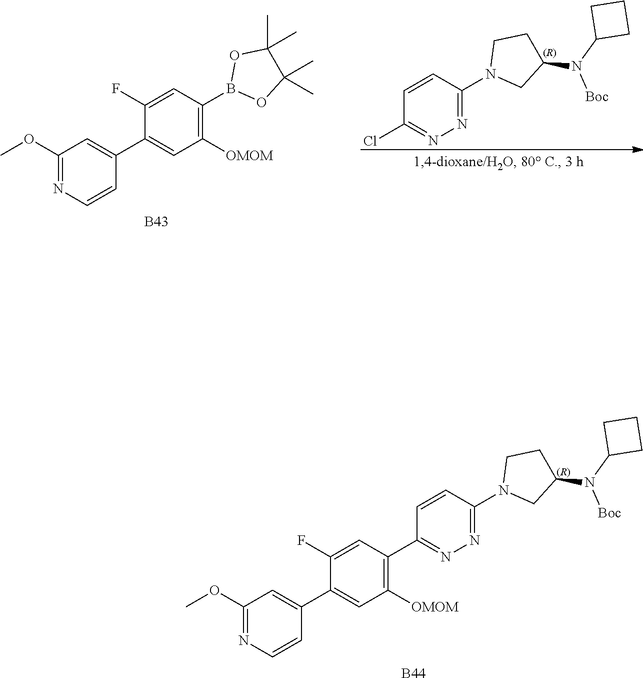

A mixture of 4-[2-fluoro-5-(methoxymethoxy)-4-(4,4,5,5-tetramethyl-1,3,2-dioxaborolan-2-yl)phenyl]-2-methoxypyridine (200 mg, 0.514 mmol, 1.2 equiv), dioxane (2 mL), water (0.4 mL), tert-butyl N-[(3R)-1-(6-chloropyridazin-3-yl)pyrrolidin-3-yl]-N-cyclobutylcarbamate (151.09 mg, 0.428 mmol, 1.0 equiv), K₃PO₄ (181.78 mg, 0.857 mmol, 2.0 equiv), and Pd(PPh₃)₄ (49.48 mg, 0.043 mmol, 0.1 equiv) was stirred 3 h at 80° C. under nitrogen atmosphere. The resulting mixture was concentrated under reduced pressure to give a residue. The residue was purified by silica gel column chromatography, eluted with PE/EA (1:1) to afford tert-butyl N-cyclobutyl-N-[(3R)-1-{6-[5-fluoro-2-(methoxymethoxy)-4-(2-methoxypyridin-4-yl)phenyl]pyridazin-3-yl}pyrrolidin-3-yl]carbamate (170 mg, 68%) as an oil. LCMS (ES, m/z): 580 [M+H]⁺.

### Synthesis of Compound 220

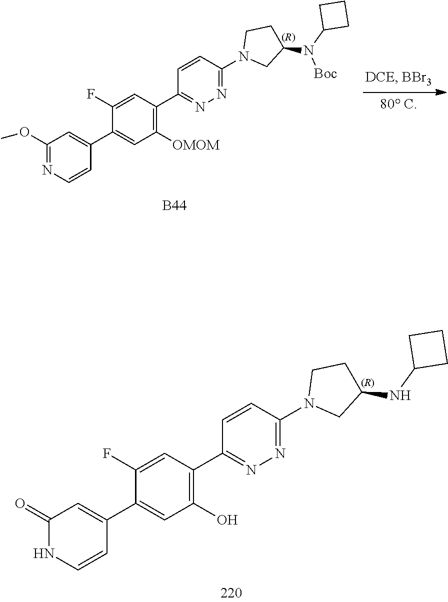

A mixture of tert-butyl N-cyclobutyl-N-[(3R)-1-{6-[5-fluoro-2-(methoxymethoxy)-4-(2-methoxypyridin-4-yl)phenyl]pyridazin-3-yl}pyrrolidin-3-yl]carbamate (100 mg, 0.173 mmol, 1.0 equiv), DCE (1 mL), and BBr₃ (129.65 mg, 0.519 mmol, 3.0 equiv) was stirred overnight at 80° C. The reaction mixture was quenched with methanol (5 mL) at room temperature, then concentrated under reduced pressure to give a residue. The residue was purified by reverse phase flash chromatography (Condition 1, Gradient 1), followed by chiral SFC (Condition 6, Gradient 1) to afford 4-(4-{6-[(3R)-3-(cyclobutylamino)pyrrolidin-1-yl]pyridazin-3-yl}-2-fluoro-5-hydroxyphenyl)-1H-pyridin-2-one (10 mg) as a solid. LCMS (ES, m/z): 422 [M+H]⁺. ¹H NMR (400 MHz, DMSO-d₆) δ 13.58 (s, 1H), 8.30 (s, 1H), 7.93 (d, J=12.3 Hz, 1H), 7.46 (d, J=6.8 Hz, 1H), 7.21 (s, 1H), 7.07 (d, J=6.8 Hz, 1H), 6.51 (s, 1H), 6.39 (d, J=6.8 Hz, 1H), 3.65 (s, 6H), 2.19 (s, 3H), 1.91 (s, 2H), 1.67 (s, 3H).

### Example 17: Synthesis of Compound 221

### Synthesis of Intermediate B45

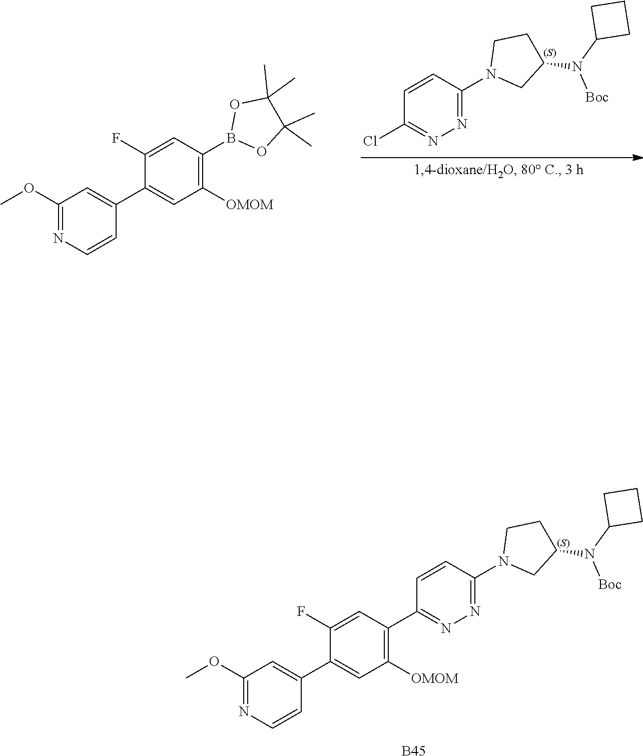

A mixture of 4-[2-fluoro-5-(methoxymethoxy)-4-(4,4,5,5-tetramethyl-1,3,2-dioxaborolan-2-yl)phenyl]-2-methoxypyridine (200 mg, 0.514 mmol, 1.2 equiv), dioxane (2 mL), water (0.4 mg), tert-butyl N-[(3S)-1-(6-chloropyridazin-3-yl)pyrrolidin-3-yl]-N-cyclobutylcarbamate (151.09 mg, 0.428 mmol, 1.0 equiv), K₃PO₄ (181.78 mg, 0.857 mmol, 2.0 equiv), and Pd(PPh₃)4 (49.48 mg, 0.043 mmol, 0.1 equiv) at room temperature under nitrogen atmosphere. The resulting mixture was stirred overnight at 80° C. under nitrogen atmosphere, then concentrated under reduced pressure to give a residue. The residue was purified by silica gel column chromatography, eluted with PE/EA (1:1) to afford tert-butyl N-cyclobutyl-N-[(3S)-1-{6-[5-fluoro-2-(methoxymethoxy)-4-(2-methoxypyridin-4-yl)phenyl]pyridazin-3-yl}pyrrolidin-3-yl]carbamate (170 mg, 68%) as an oil. LCMS (ES, m/z): 580 [M+H]⁺.

### Synthesis of Compound 221

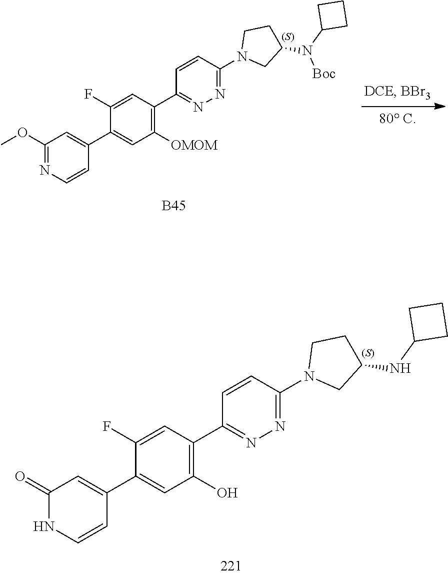

A mixture of tert-butyl N-cyclobutyl-N-[(3S)-1-{6-[5-fluoro-2-(methoxymethoxy)-4-(2-methoxypyridin-4-yl)phenyl]pyridazin-3-yl}pyrrolidin-3-yl]carbamate (100 mg, 0.173 mmol, 1.0 equiv), DCE (1 mL), and BBr₃ (129.65 mg, 0.519 mmol, 3.0 equiv) was stirred overnight at 80° C. The reaction mixture was quenched with methanol (5 mL) at room temperature, then concentrated under reduced pressure to give a residue. The residue was purified by reverse phase flash chromatography (Condition 1, Gradient 1), followed by chiral SFC (Condition 6, Gradient 1) to afford 4-(4-{6-[(3S-3-(cyclobutylamino)pyrrolidin-1-yl]pyridazin-3-yl}-2-fluoro-5-hydroxyphenyl)-1H-pyridin-2-one (10 mg) as a solid. LCMS (ES, m/z): 422 [M+H]⁺. ¹H NMR (400 MHz, DMSO-d₆) δ 13.61 (s, 1H), 11.69 (s, 1H), 8.28 (d, J=9.5 Hz, 1H), 7.91 (d, J=12.2 Hz, 1H), 7.46 (s, 1H), 7.17 (d, J=9.7 Hz, 1H), 7.06 (s, 1H), 6.50 (s, 1H), 6.39 (s, 1H), 3.64 (s, 3H), 2.14 (s, 4H), 1.86 (s, 2H), 1.73 (s, 2H), 1.61 (s, 3H).

### Example 18: Synthesis of Compound 222

### Synthesis of Intermediate B46

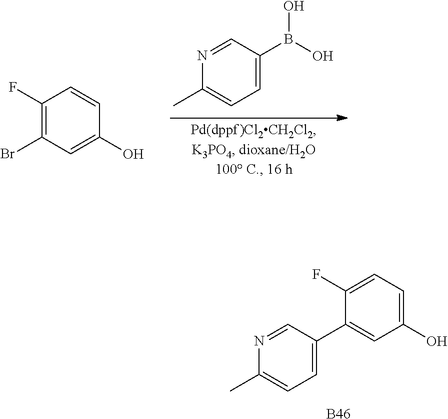

To a stirred mixture of 3-bromo-4-fluorophenol (4 g, 20.943 mmol, 1 equiv) and 6-methylpyridin-3-ylboronic acid (3.44 g, 25.132 mmol, 1.2 equiv) in a mixture of dioxane (60 mL) and H₂O (12 mL) was added K₃PO₄ (13.34 g, 62.829 mmol, 3 equiv) and Pd(dppf)Cl₂·CH₂Cl₂ (1.71 g, 2.094 mmol, 0.1 equiv) at room temperature under nitrogen atmosphere. The resulting mixture was stirred for 15 h at 100° C. under nitrogen atmosphere, then concentrated under reduced pressure to give a residue. The residue was purified by silica gel column chromatography, eluted with PE/EA (3:1) to afford 4-fluoro-3-(6-methylpyridin-3-yl)phenol (3.2 g, 75%) as a solid. LCMS (ES, m/z): 204 [M+H]⁺.

### Synthesis of Intermediate B47

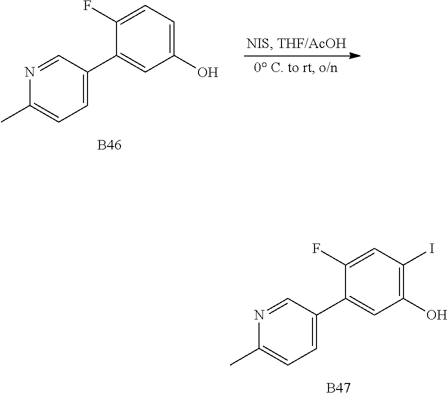 [SMILES: "B46" ; Cc1ccc(-c2cc(O)ccc2F)cn1 ; "B47" ; Cc1ccc(-c2cc(O)c(I)cc2F)cn1]

To a stirred mixture of 4-fluoro-3-(6-methylpyridin-3-yl)phenol (3.2 g, 15.747 mmol, 1 equiv) in THF (60 mL) and AcOH (60 mL) was added NIS (3.07 g, 13.624 mmol, 1.0 equiv) in portions at 0° C. The resulting mixture was stirred overnight at room temperature, then concentrated under reduced pressure to give a residue. The residue was purified by reverse phase flash chromatography (Condition 4, Gradient 1) to afford 4-fluoro-2-iodo-5-(6-methylpyridin-3-yl)phenol (2.4 g, 46%) as a solid. LCMS (ES, m/z): 330 [M+H]⁺.

### Synthesis of Intermediate B48

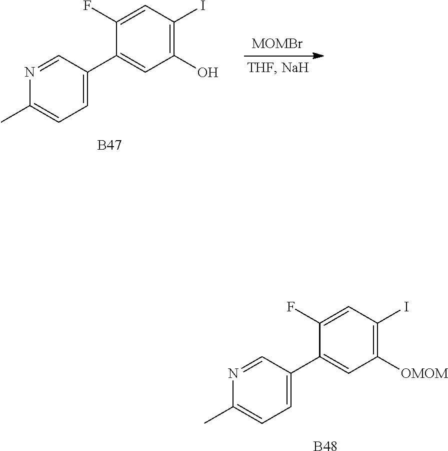

To a solution of 4-fluoro-2-iodo-5-(6-methylpyridin-3-yl)phenol (2.4 g, 7.292 mmol, 1 equiv) in THE (24 mL) was added NaH (0.35 g, 8.750 mmol, 1.2 equiv, 60%) at 0° C. The resulting mixture was stirred for 30 min. To the reaction mixture was added bromo(methoxy)methane (0.82 g, 6.563 mmol, 0.9 equiv) dropwise at 0° C. The resulting mixture was allowed to warm to room temperature and stirred for 2 h, then quenched with methanol. The resulting mixture was concentrated under reduced pressure to give a residue. The residue was purified by silica gel column chromatography, eluted with PE/EA (5:1) to afford 5-[2-fluoro-4-iodo-5-(methoxymethoxy)phenyl]-2-methylpyridine (1.2 g, 44%) as a solid. LCMS (ES, m/z): 374 [M+H]⁺.

### Synthesis of Intermediate B49

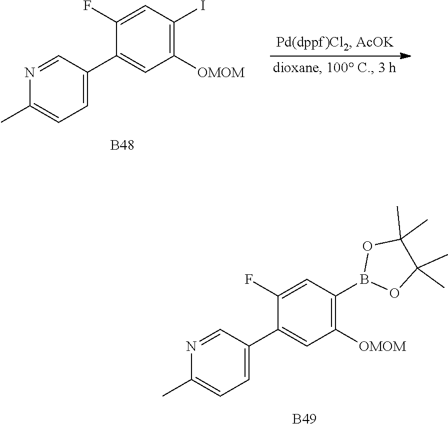

To a stirred mixture of 5-[2-fluoro-4-iodo-5-(methoxymethoxy)phenyl]-2-methylpyridine (1.2 g, 3.216 mmol, 1 equiv) and bis(pinacolato)diboron (1.22 g, 4.824 mmol, 1.5 equiv) in dioxane (24 mL) was added AcOK (0.95 g, 9.648 mmol, 3 equiv) and Pd(dppf)Cl₂ (0.24 g, 0.322 mmol, 0.1 equiv) at room temperature under nitrogen atmosphere. The resulting mixture was stirred for 3 h at 100° C. under nitrogen atmosphere, then concentrated under reduced pressure to give a residue. The residue was purified by silica gel column chromatography, eluted with PE/EA (3:1) to afford 5-[2-fluoro-5-(methoxymethoxy)-4-(4,4,5,5-tetramethyl-1,3,2-dioxaborolan-2-yl)phenyl]-2-methylpyridine (0.9 g, 37%) as an oil. LCMS (ES, m/z): 374 [M+H]⁺.

### Synthesis of Intermediate B50

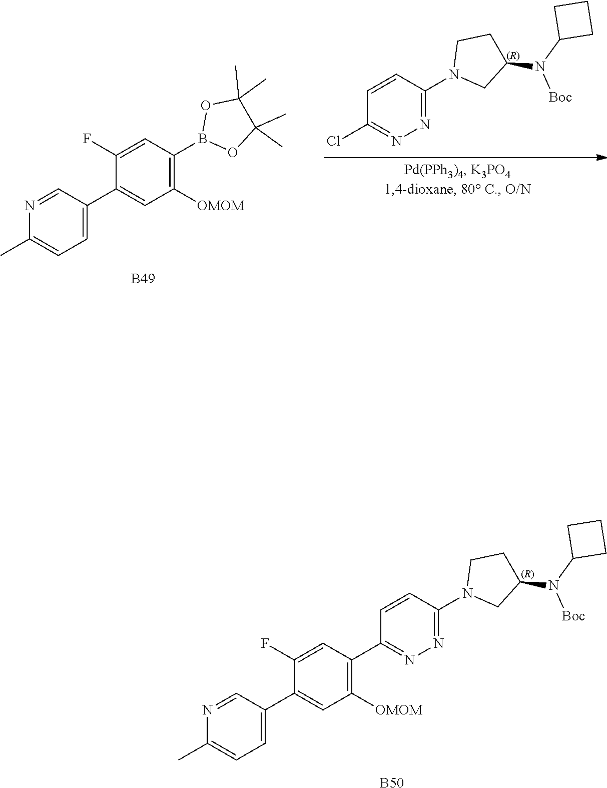

To a stirred mixture of 5-[2-fluoro-5-(methoxymethoxy)-4-(4,4,5,5-tetramethyl-1,3,2-dioxaborolan-2-yl)phenyl]-2-methylpyridine (200 mg, 0.536 mmol, 1 equiv) and tert-butyl N-[(3R)-1-(6-chloropyridazin-3-yl)pyrrolidin-3-yl]-N-cyclobutylcarbamate (226.90 mg, 0.643 mmol, 1.2 equiv) in 1,4-dioxane (4 mL) was added K₃PO₄ (341.23 mg, 1.608 mmol, 3 equiv) and Pd(PPh₃)₄(61.92 mg, 0.054 mmol, 0.1 equiv) at room temperature under nitrogen atmosphere. The resulting mixture was stirred overnight at 80° C. under nitrogen atmosphere, then concentrated under reduced pressure to give a residue. The residue was purified by silica gel column chromatography, eluted with CH₂Cl₂/MeOH (20:1) to afford tert-butyl N-cyclobutyl-N-[(3R)-1-{6-[5-fluoro-2-(methoxymethoxy)-4-(6-methylpyridin-3-yl)phenyl]pyridazin-3-yl}pyrrolidin-3-yl]carbamate (100 mg, 23%) as a solid. LCMS (ES, m/z): 564 [M+H]⁺.

### Synthesis of Compound 222

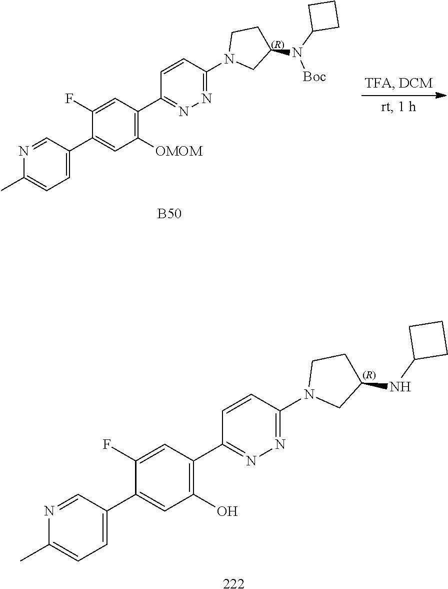

To a stirred solution of tert-butyl N-cyclobutyl-N-[(3R)-1-{6-[5-fluoro-2-(methoxymethoxy)-4-(6-methylpyridin-3-yl)phenyl]pyridazin-3-yl}pyrrolidin-3-yl]carbamate (100 mg, 0.124 mmol, 1 equiv, 70%) in DCM (1 mL) was added TFA (1 mL) at room temperature. The resulting mixture was stirred for 1 h at room temperature, then concentrated under reduced pressure to give a residue. The residue was purified by Prep-HPLC (Condition 5, Gradient 1) to afford 2-{6-[(3S)-3-(tert-butylamino)pyrrolidin-1-yl]pyridazin-3-yl}-4-fluoro-5-(6-methoxypyridazin-4-yl)phenol (16.9 mg, 31%) as a solid. LCMS (ES, m/z): 420 [M+H]⁺. ¹H NMR (400 MHz, Methanol-d₄) δ 8.65 (d, J=2.5 Hz, 1H), 8.13 (d, J=9.8 Hz, 1H), 7.98 (dt, J=8.1, 1.7 Hz, 1H), 7.70 (d, J=12.0 Hz, 1H), 7.43 (d, J=8.1 Hz, 1H), 7.18 (d, J=9.7 Hz, 1H), 7.10 (d, J=6.9 Hz, 1H), 3.85-3.68 (m, 2H), 3.63-3.49 (m, 2H), 3.40 (p, J=7.2, 6.7 Hz, 2H), 2.61 (s, 3H), 2.30 (ddd, J=13.2, 8.6, 4.1 Hz, 3H), 1.97 (dq, J=14.5, 7.5 Hz, 1H), 1.91-1.77 (m, 2H), 1.80-1.67 (m, 2H).

### Example 19: Synthesis of Compound 223

### Synthesis of Intermediate B51

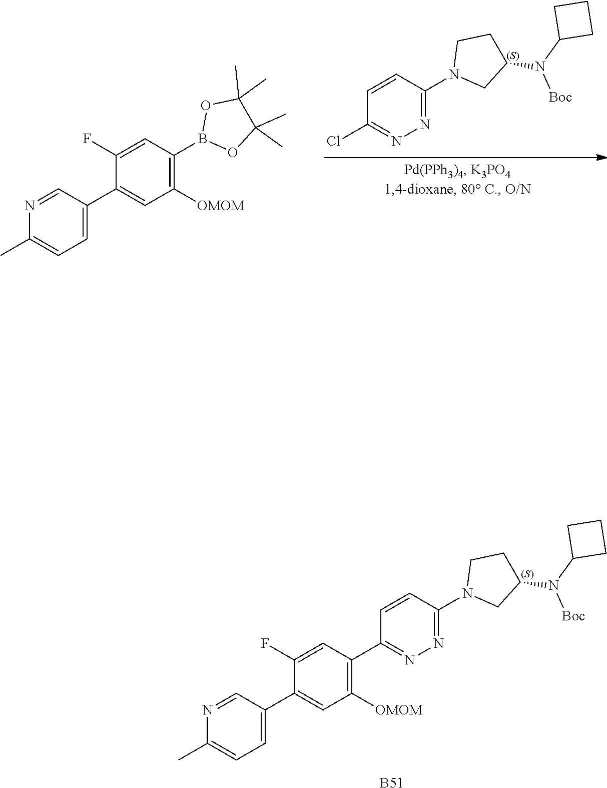

To a stirred mixture of 5-[2-fluoro-5-(methoxymethoxy)-4-(4,4,5,5-tetramethyl-1,3,2-dioxaborolan-2-yl)phenyl]-2-methylpyridine (200 mg, 0.536 mmol, 1 equiv) and tert-butyl N-[(3S)-1-(6-chloropyridazin-3-yl)pyrrolidin-3-yl]-N-cyclobutylcarbamate (226.90 mg, 0.643 mmol, 1.2 equiv) in 1,4-dioxane (4 mL) was added K₃PO₄ (341.23 mg, 1.608 mmol, 3 equiv) and Pd(PPh₃)₄(61.92 mg, 0.054 mmol, 0.1 equiv) at room temperature under nitrogen atmosphere. The resulting mixture was stirred overnight at 80° C. under nitrogen atmosphere, then concentrated under reduced pressure to give a residue. The residue was purified by silica gel column chromatography, eluted with CH₂Cl₂/MeOH (20:1) to afford tert-butyl N-cyclobutyl-N-[(3S)-1-{6-[5-fluoro-2-(methoxymethoxy)-4-(6-methylpyridin-3-yl)phenyl]pyridazin-3-yl}pyrrolidin-3-yl]carbamate (100 mg, 23%) as a solid. LCMS (ES, m/z): 564 [M+H]⁺.

### Synthesis of Compound 223

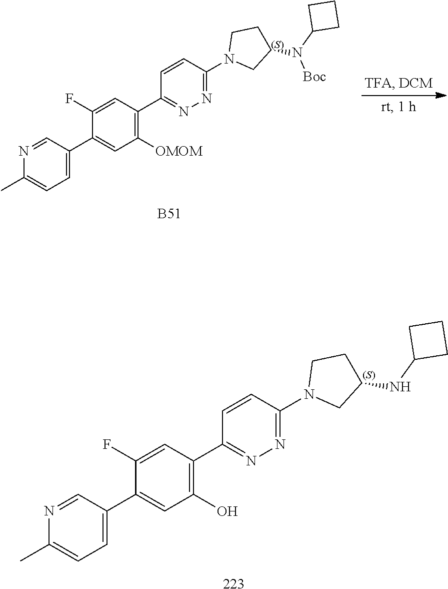

To a stirred solution of tert-butyl N-cyclobutyl-N-[(3S)-1-{6-[5-fluoro-2-(methoxymethoxy)-4-(6-methylpyridin-3-yl)phenyl]pyridazin-3-yl}pyrrolidin-3-yl]carbamate (100 mg, 0.124 mmol, 1 equiv, 70%) in DCM (1 mL) was added TFA (1 mL) at room temperature. The resulting mixture was stirred for 1 h at room temperature, then concentrated under reduced pressure to give a residue. The residue was purified by Prep-HPLC (Condition 5, Gradient 1) to afford 2-{6-[(3S)-3-(cyclobutylamino)pyrrolidin-1-yl]pyridazin-3-yl}-4-fluoro-5-(6-methylpyridin-3-yl)phenol (25.7 mg, 49%) as a solid. LCMS (ES, m/z): 420 [M+H]⁺. ¹H NMR (400 MHz, Methanol-d₄) δ 8.64 (d, J=2.4 Hz, 1H), 8.13 (d, J=9.7 Hz, 1H), 8.01-7.94 (m, 1H), 7.69 (d, J=12.1 Hz, 1H), 7.43 (d, J=8.1 Hz, 1H), 7.17 (d, J=9.7 Hz, 1H), 7.09 (d, J=6.9 Hz, 1H), 3.84-3.69 (m, 2H), 3.62-3.49 (m, 2H), 3.39 (q, J=8.9, 8.4 Hz, 2H), 2.61 (s, 3H), 2.30 (ddd, J=13.1, 8.9, 4.1 Hz, 3H), 1.97 (dq, J=14.5, 7.5 Hz, 1H), 1.92-1.80 (m, 2H), 1.74 (ddt, J=12.7, 6.7, 3.2 Hz, 2H).

### Example 20: Synthesis of Compound 224

### Synthesis of Intermediate B52

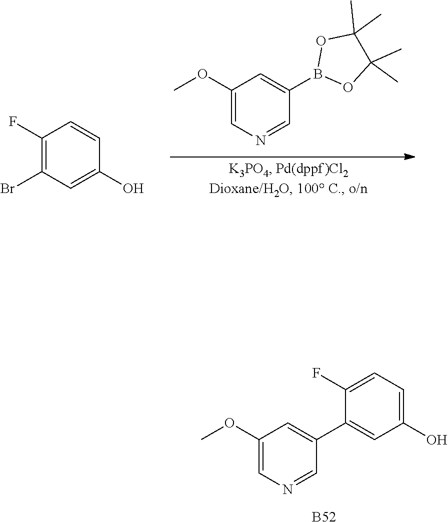

To a stirred mixture of 3-bromo-4-fluorophenol (1.5 g, 7.853 mmol, 1 equiv) and 3-methoxy-5-(4,4,5,5-tetramethyl-1,3,2-dioxaborolan-2-yl)pyridine (2.22 g, 9.424 mmol, 1.2 equiv) in a mixture of 1,4-dioxane and H₂O (25 mL/5 mL) was added K₃PO₄ (5.00 g, 23.559 mmol, 3 equiv) and Pd(dppf)Cl₂ (0.29 g, 0.393 mmol, 0.05 equiv) at room temperature under nitrogen atmosphere. The resulting mixture was stirred overnight at 100° C. under nitrogen atmosphere, then concentrated under vacuum to give a residue. The residue was purified by silica gel column chromatography, eluted with PE/EA (1:1) to afford 4-fluoro-3-(5-methoxypyridin-3-yl)phenol (1.2 g, 70%) as a solid. LCMS (ES, m/z): 220 [M+H]⁺.

### Synthesis of Intermediate B53

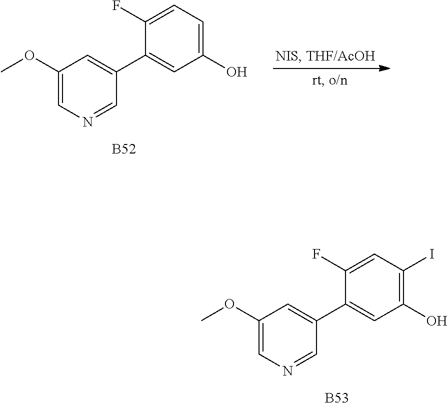 [SMILES: "B52" ; COc1cncc(-c2cc(O)ccc2F)c1 ; "B53" ; COc1cncc(-c2cc(O)c(I)cc2F)c1]

To a stirred mixture of 4-fluoro-3-(5-methoxypyridin-3-yl)phenol (1.6 g, 7.299 mmol, 1 equiv) in THE and AcOH (32 mL/32 mL) was added NIS (1.64 g, 7.299 mmol, 1.0 equiv) in portions at room temperature. The resulting mixture was stirred overnight at room temperature, then concentrated under vacuum to give a residue. The residue was purified by silica gel column chromatography, eluted with PE/EA (5:1), then triturated with methanol (10 mL) to afford 4-fluoro-2-iodo-5-(5-methoxypyridin-3-yl)phenol (0.85 g, 29%) as a solid. LCMS (ES, m/z): 346 [M+H]⁺.

### Synthesis of Intermediate B54

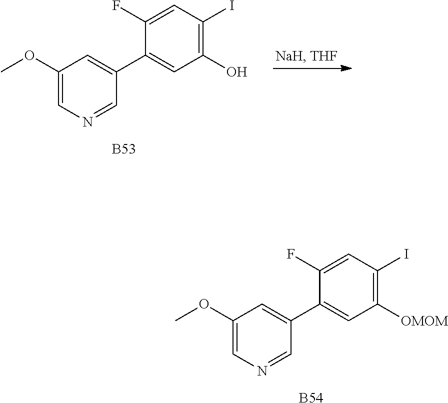

To a stirred solution of 4-fluoro-2-iodo-5-(5-methoxypyridin-3-yl)phenol (0.7 g, 2.028 mmol, 1.0 equiv) in THE (10 mL) was added NaH (0.06 g, 2.434 mmol, 1.2 equiv) in portions at 0° C. under nitrogen atmosphere, then stirred for 30 min at 0° C. under nitrogen atmosphere. To the resulting mixture was added bromo(methoxy)methane (0.22 g, 1.724 mmol, 0.85 equiv) dropwise at 0° C. The resulting mixture was stirred for an additional 1 h at 0° C. The reaction mixture was quenched with methanol (10 mL) at 0° C. and concentrated under reduced pressure to give a residue. The residue was purified by silica gel column chromatography, eluted with PE/EA (5:1) to afford 3-[2-fluoro-4-iodo-5-(methoxymethoxy)phenyl]-5-methoxypyridine (0.4 g, 51%) as a solid. LCMS (ES, m/z): 390 [M+H]⁺.

### Synthesis of Intermediate B55

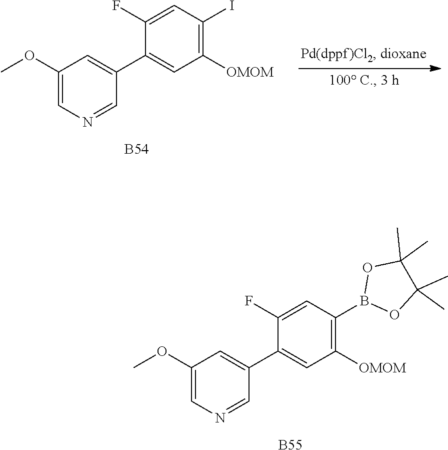

A mixture of 3-[2-fluoro-4-iodo-5-(methoxymethoxy)phenyl]-5-methoxypyridine (400 mg, 1.028 mmol, 1.0 equiv), 4,4,5,5-tetramethyl-2-(tetramethyl-1,3,2-dioxaborolan-2-yl)-1,3,2-dioxaborolane (1044.04 mg, 4.112 mmol, 4.0 equiv), AcOK (302.62 mg, 3.084 mmol, 3.0 equiv), dioxane (8 mL), and Pd(dppf)Cl₂ (75.21 mg, 0.103 mmol, 0.1 equiv) was stirred for 4 h at 100° C. under nitrogen atmosphere. The reaction mixture was concentrated and purified by silica gel column chromatography, eluted with PE/EA (3:1) to afford 3-[2-fluoro-5-(methoxymethoxy)-4-(4,4,5,5-tetramethyl-1,3,2-dioxaborolan-2-yl)phenyl]-5-methoxypyridine (360 mg, 90%) as an oil. LCMS (ES, m/z): 390 [M+H]⁺.

### Synthesis of Intermediate B56

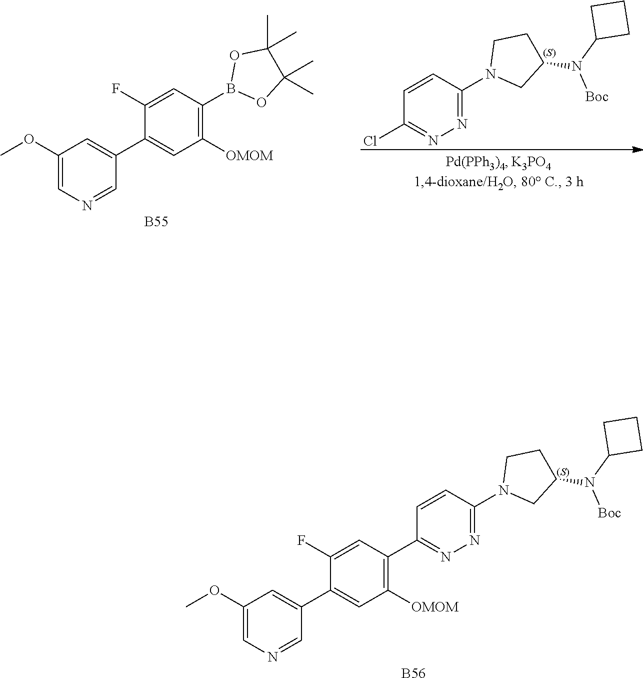

A mixture of tert-butyl N-[(3S)-1-(6-chloropyridazin-3-yl)pyrrolidin-3-yl]-N-cyclobutylcarbamate (135.98 mg, 0.385 mmol, 1.0 equiv), 3-[2-fluoro-5-(methoxymethoxy)-4-(4,4,5,5-tetramethyl-1,3,2-dioxaborolan-2-yl)phenyl]-5-methoxypyridine (180 mg, 0.462 mmol, 1.2 equiv), K₃PO₄ (163.60 mg, 0.770 mmol, 2.0 equiv), dioxane (2 mL), water (0.4 mL) and Pd(PPh₃)₄(44.53 mg, 0.039 mmol, 0.1 equiv) was stirred for 3 h at 80° C. under nitrogen atmosphere. The reaction mixture was concentrated and the residue was purified by silica gel column chromatography, eluted with PE/EA (1:1) to afford tert-butyl N-cyclobutyl-N-[(3S)-1-{6-[5-fluoro-2-(methoxymethoxy)-4-(5-methoxypyridin-3-yl)phenyl]pyridazin-3-yl}pyrrolidin-3-yl]carbamate (100 mg, 45%) as an oil. LCMS (ES, m/z): 580 [M+H]⁺.

### Synthesis of Compound 224

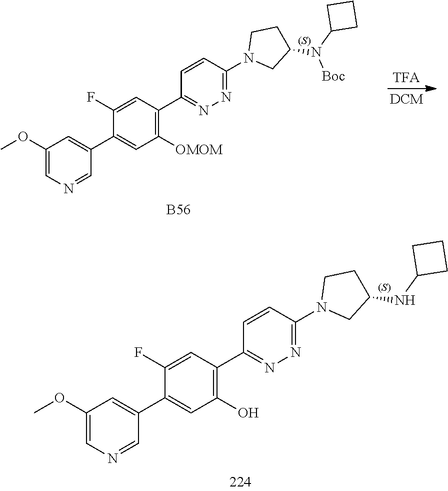

A mixture of tert-butyl N-cyclobutyl-N-[(3S)-1-{6-[5-fluoro-2-(methoxymethoxy)-4-(5-methoxypyridin-3-yl)phenyl]pyridazin-3-yl}pyrrolidin-3-yl]carbamate (100 mg, 0.173 mmol, 1 equiv), DCM (1 mL), and TFA (1 mL) was stirred for 3 h at room temperature. The resulting mixture was concentrated to give a residue. The residue was purified by reverse phase flash chromatography (Condition 1, Gradient 1) to afford 2-{6-[(3S)-3-(cyclobutylamino)pyrrolidin-1-yl]pyridazin-3-yl}-4-fluoro-5-(5-methoxypyridin-3-yl)phenol (15 mg, 20%) as a solid. LCMS (ES, m/z): 436 [M+H]⁺. ¹H NMR (400 MHz, DMSO-d₆) δ 13.71 (s, 1H), 8.40 (t, J=1.9 Hz, 1H), 8.34 (d, J=2.9 Hz, 1H), 8.29 (d, J=9.7 Hz, 1H), 7.94 (d, J=12.2 Hz, 1H), 7.58 (s, 1H), 7.21-7.14 (m, 2H), 3.91 (s, 3H), 3.68-3.59 (m, 2H), 3.52 (s, 1H), 3.44-3.37 (m, 1H), 3.31-3.23 (m, 2H), 2.14 (s, 3H), 1.84 (dd, J=12.4, 6.5 Hz, 1H), 1.71 (s, 2H), 1.60 (s, 2H).

### Example 21: Synthesis of Compound 225

### Synthesis of Intermediate B57

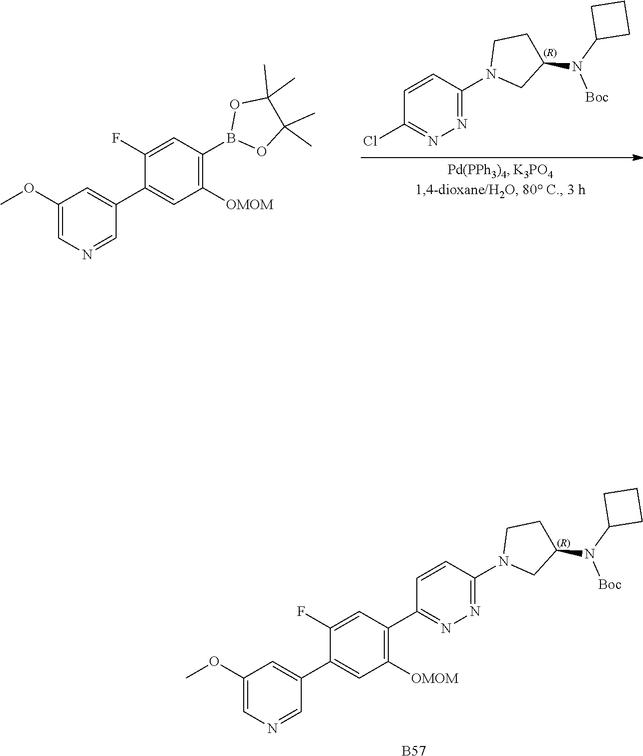

A mixture of 3-[2-fluoro-5-(methoxymethoxy)-4-(4,4,5,5-tetramethyl-1,3,2-dioxaborolan-2-yl)phenyl]-5-methoxypyridine (180 mg, 0.462 mmol, 1.2 equiv), tert-butyl N-[(3R)-1-(6-chloropyridazin-3-yl)pyrrolidin-3-yl]-N-cyclobutylcarbamate (135.98 mg, 0.385 mmol, 1.0 equiv), K₃PO₄ (163.60 mg, 0.770 mmol, 2.0 equiv), dioxane (2 mL), water (0.4 mL) and Pd(PPh₃)₄(44.53 mg, 0.039 mmol, 0.1 equiv) was stirred for 3 h at 80° C. under nitrogen atmosphere. The mixture was concentrated to give a residue and the residue was purified by silica gel column chromatography, eluted with PE/EA (1:1) to afford tert-butyl N-cyclobutyl-N-[(3R)-1-{6-[5-fluoro-2-(methoxymethoxy)-4-(5-methoxypyridin-3-yl)phenyl]pyridazin-3-yl}pyrrolidin-3-yl]carbamate (100 mg, 45%) as an oil. LCMS (ES, m/z): 580 [M+H]⁺.

### Synthesis of Compound 225

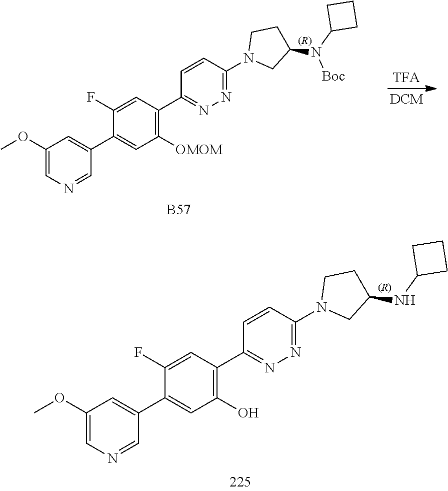

A mixture of tert-butyl N-cyclobutyl-N-[(3R)-1-{6-[5-fluoro-2-(methoxymethoxy)-4-(5-methoxypyridin-3-yl)phenyl]pyridazin-3-yl}pyrrolidin-3-yl]carbamate (100 mg, 0.173 mmol, 1 equiv), DCM (1 mL) and TFA (1 mL) was stirred for 3 h at room temperature. The reaction mixture was concentrated in vacuum to give a residue. The residue was purified by reverse phase flash chromatography (Condition 1, Gradient 1) to afford 2-{6-[(3R)-3-(cyclobutylamino)pyrrolidin-1-yl]pyridazin-3-yl}-4-fluoro-5-(5-methoxypyridin-3-yl)phenol (20 mg, 27%) as a solid. LCMS (ES, m/z): 436 [M+H]⁺. ¹H NMR (400 MHz, DMSO-d₆) δ 13.71 (s, 1H), 8.40 (d, J=2.0 Hz, 1H), 8.34 (d, J=2.8 Hz, 1H), 8.30 (d, J=9.6 Hz, 1H), 7.94 (d, J=12.2 Hz, 1H), 7.58 (s, 1H), 7.21-7.14 (m, 2H), 3.91 (s, 3H), 3.64 (s, 2H), 3.52 (s, 1H), 3.40 (q, J=5.6 Hz, 1H), 3.32-3.20 (m, 2H), 2.14 (s, 3H), 1.84 (s, 1H), 1.71 (s, 2H), 1.59 (q, J=9.5 Hz, 2H).

### Example 22: Synthesis of Compound 226

### Synthesis of Intermediate B58

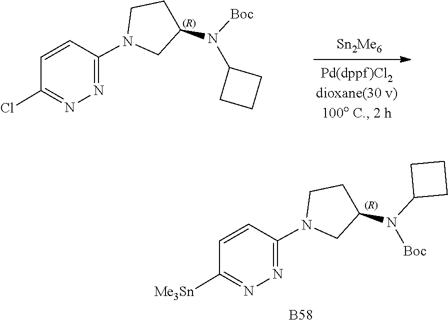

A mixture of tert-butyl N-[(3R)-1-(6-chloropyridazin-3-yl)pyrrolidin-3-yl]-N-cyclobutylcarbamate (3.0 g, 8.502 mmol, 1 equiv), dioxane (100 mL), Sn₂Me₆ (5.57 g, 17.004 mmol, 2.0 equiv), and Pd(dppf)Cl₂ (0.62 g, 0.850 mmol, 0.1 equiv) was stirred for 2 h at 100° C. under nitrogen atmosphere. The resulting mixture was concentrated under reduced pressure to give a residue. The residue was purified by basic Al₂O₃ column eluted with PE/EA (3:1) to afford tert-butyl N-cyclobutyl-N-[(3R)-1-[6-(trimethylstannyl)pyridazin-3-yl]pyrrolidin-3-yl]carbamate (1.0 g, 24%) as an oil. LCMS (ES, m/z): 483 [M+H]⁺.

### Synthesis of Intermediate B59

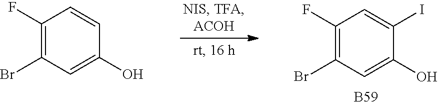 [SMILES: Oc1ccc(F)c(Br)c1 ; "B59" ; Oc1cc(Br)c(F)cc1I]

To a stirred mixture of 3-bromo-4-fluorophenol (13 g, 68.063 mmol, 1 equiv) in THE (130 mL) and AcOH (130 mL) was added NIS (15.31 g, 68.063 mmol, 1 equiv) in portions at 0° C. The resulting mixture was stirred for 16 h at room temperature, then concentrated under reduced pressure to give a residue. The residue was purified by reverse phase flash chromatography (Condition 4, Gradient 1) to afford 5-bromo-4-fluoro-2-iodophenol (2.7 g) as a solid. LCMS (ES, m/z): 317 [M+H]⁺.

### Synthesis of Intermediate B60

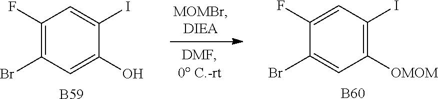

To a stirred mixture of 5-bromo-4-fluoro-2-iodophenol (2.7 g, 8.520 mmol, 1 equiv) and DIEA (3.30 g, 25.560 mmol, 3 equiv) in DCM (24 mL) was added bromo(methoxy)methane (1.17 g, 9.372 mmol, 1.1 equiv) dropwise at 0° C. The resulting mixture was stirred for 4 h at room temperature, then concentrated under reduced pressure to give a residue. The residue was purified by silica gel column chromatography, eluted with PE/EA (5:1) to afford 1-bromo-2-fluoro-4-iodo-5-(methoxymethoxy)benzene (2.4 g, 78%) as a solid. LCMS (ES, m/z): 361 [M+H]⁺.

### Synthesis of Intermediate B61

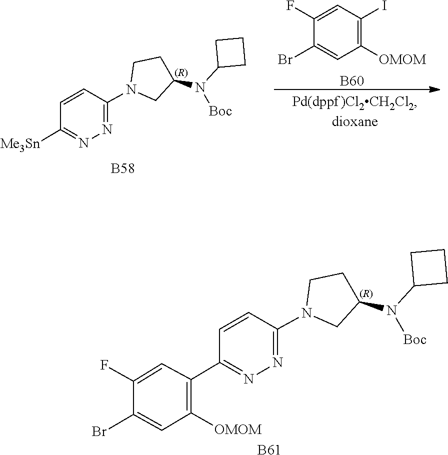

A mixture of tert-butyl N-cyclobutyl-N-[(3R)-1-[6-(trimethylstannyl)pyridazin-3-yl]pyrrolidin-3-yl]carbamate (1.0 g, 2.078 mmol, 1.2 equiv), dioxane (10 mL), 1-bromo-2-fluoro-4-iodo-5-(methoxymethoxy)benzene (0.63 g, 1.732 mmol, 1.0 equiv), and Pd(dppf)Cl₂ (0.13 g, 0.173 mmol, 0.1 equiv) was stirred for 4 h at 80° C. under nitrogen atmosphere. The resulting mixture was concentrated under reduced pressure to give a residue. The residue was purified by silica gel column chromatography, eluted with PE/EA (1:1) to afford tert-butyl N-[(3R)-1-{6-[4-bromo-5-fluoro-2-(methoxymethoxy)phenyl]pyridazin-3-yl}pyrrolidin-3-yl]-N-cyclobutylcarbamate (0.4 g, 42%) as a solid. LCMS (ES, m/z): 551 [M+H]⁺.

### Synthesis of Intermediate B62

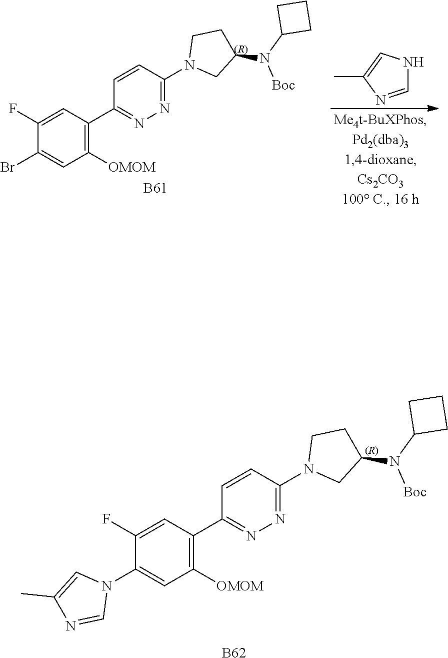

A mixture of tert-butyl N-[(3R)-1-{6-[4-bromo-5-fluoro-2-(methoxymethoxy)phenyl]pyridazin-3-yl}pyrrolidin-3-yl]-N-cyclobutylcarbamate (80 mg, 0.145 mmol, 1.0 equiv), 4(5)-methylimidazole (23.82 mg, 0.290 mmol, 2.0 equiv), Cs₂CO₃ (141.80 mg, 0.435 mmol, 3.0 equiv), di-tert-butyl([2,3,4,5-tetramethyl-6-[2,4,6-tris(propan-2-yl)phenyl]phenyl])phosphane (13.95 mg, 0.029 mmol, 0.2 equiv), dioxane (1 mL) and Pd₂(dba)₃ (15.02 mg, 0.014 mmol, 0.1 equiv) was stirred for 16 h at 100° C. under nitrogen atmosphere. The resulting mixture was concentrated under reduced pressure to give a residue. The residue was purified by silica gel column chromatography, eluted with CH₂Cl₂/MeOH (20:1) to afford tert-butyl N-cyclobutyl-N-[(3R)-1-{6-[5-fluoro-2-(methoxymethoxy)-4-(4-methylimidazol-1-yl)phenyl]pyridazin-3-yl}pyrrolidin-3-yl]carbamate (50 mg, 62%) as a solid. LCMS (ES, m/z): 553 [M+H]⁺.

### Synthesis of Compound 226

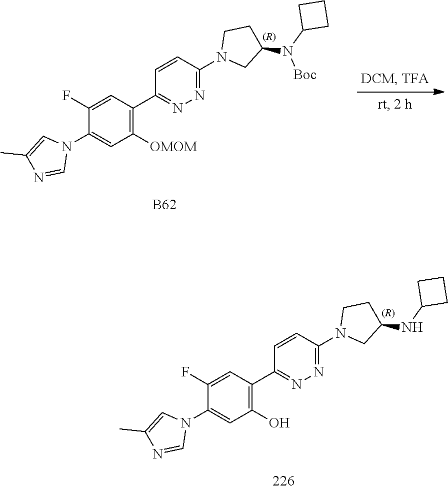

A mixture of tert-butyl N-cyclobutyl-N-[(3R)-1-{6-[5-fluoro-2-(methoxymethoxy)-4-(4-methylimidazol-1-yl)phenyl]pyridazin-3-yl}pyrrolidin-3-yl]carbamate (50 mg, 0.090 mmol, 1 equiv), DCM (1 mL) and TFA (1 mL) was stirred for 2 h at room temperature. The resulting mixture was concentrated under reduced pressure to give a residue. The residue was purified by reverse phase flash chromatography (Condition 1, Gradient 1) to afford 2-{6-[(3R)-3-(cyclobutylamino)pyrrolidin-1-yl]pyridazin-3-yl}-4-fluoro-5-(4-methylimidazol-1-yl)phenol (10 mg, 27%) as a solid. LCMS (ES, m/z): 409 [M+H]⁺. ¹H NMR (400 MHz, Methanol-d₄) δ 8.13 (d, J=9.8 Hz, 1H), 7.96 (t, J=1.7 Hz, 1H), 7.82 (d, J=12.5 Hz, 1H), 7.25 (s, 1H), 7.19 (d, J=9.8 Hz, 1H), 7.12 (d, J=6.8 Hz, 1H), 3.81 (dd, J=10.8, 6.4 Hz, 1H), 3.75 (d, J=7.5 Hz, 1H), 3.58 (dt, J=12.6, 6.9 Hz, 2H), 3.43 (dq, J=11.8, 6.4, 5.1 Hz, 2H), 2.32 (s, 2H), 2.36-2.26 (m, 4H), 2.06-1.83 (m, 3H), 1.82-1.69 (m, 2H).

### Example 23: Synthesis of Compound 227

### Synthesis Intermediate B63

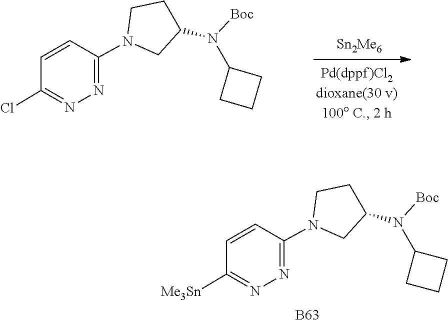

A mixture of tert-butyl N-[(3 S)-1-(6-chloropyridazin-3-yl)pyrrolidin-3-yl]-N-cyclobutylcarbamate (3.0 g, 8.502 mmol, 1.0 equiv), dioxane (100 mL), Sn₂Me₆ (5.57 g, 17.004 mmol, 2.0 equiv) and Pd(dppf)Cl₂ (0.62 g, 0.850 mmol, 0.1 equiv) was stirred for 2 h at 100° C. under nitrogen atmosphere. The resulting mixture was concentrated under reduced pressure to give a residue. The residue was purified by basic Al₂O₃ column eluted with PE/EA (3:1) to afford tert-butyl N-cyclobutyl-N-[(3S)-1-[6-(trimethylstannyl)pyridazin-3-yl]pyrrolidin-3-yl]carbamate (1.0 g, 24%) as an oil. LCMS (ES, m/z): 483 [M+H]⁺.

### Synthesis of Intermediate B64

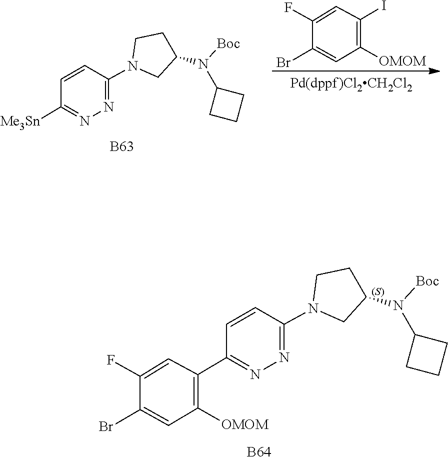

A mixture of tert-butyl N-cyclobutyl-N-[(3S)-1-[6-(trimethylstannyl)pyridazin-3-yl]pyrrolidin-3-yl]carbamate (0.8 g, 1.662 mmol, 1.2 equiv), dioxane (10 mL), 1-bromo-2-fluoro-4-iodo-5-(methoxymethoxy)benzene (0.50 g, 1.385 mmol, 1.0 equiv), and Pd(dppf)Cl₂CH₂Cl₂ (0.11 g, 0.139 mmol, 0.1 equiv) was stirred for 4 h at 80° C. under nitrogen atmosphere. The resulting mixture was concentrated under reduced pressure to give a residue. The residue was purified by silica gel column chromatography, eluted with PE/EA (1:1) to afford tert-butyl N-[(3S)-1-{6-[4-bromo-5-fluoro-2-(methoxymethoxy)phenyl]pyridazin-3-yl}pyrrolidin-3-yl]-N-cyclobutylcarbamate (0.43 g, 56%) as a solid. LCMS (ES, m/z): 551 [M+H]⁺.

### Synthesis of Intermediate B65

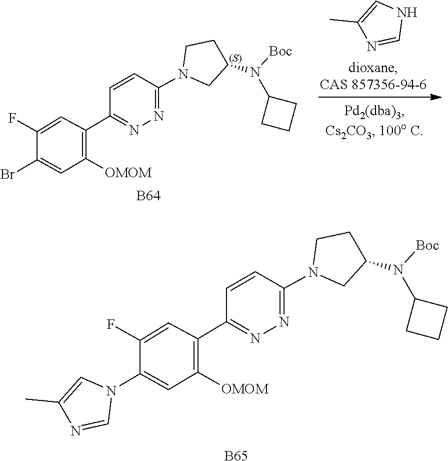

A mixture of tert-butyl N-[(3 S)-1-{6-[4-bromo-5-fluoro-2-(methoxymethoxy)phenyl]pyridazin-3-yl}pyrrolidin-3-yl]-N-cyclobutylcarbamate (80 mg, 0.145 mmol, 1.0 equiv), dioxane (1 mL), 4(5)-methylimidazole (14.29 mg, 0.174 mmol, 1.2 equiv), Cs₂CO₃ (141.80 mg, 0.435 mmol, 3.0 equiv), di-tert-butyl([2,3,4,5-tetramethyl-6-[2,4,6-tris(propan-2-yl)phenyl]phenyl])phosphane (13.95 mg, 0.029 mmol, 0.2 equiv), and Pd₂(dba)₃ (13.28 mg, 0.014 mmol, 0.1 equiv) was stirred for 16 h at 100° C. under nitrogen atmosphere. The resulting mixture was concentrated under reduced pressure to give a residue. The residue was purified by silica gel column chromatography, eluted with CH₂Cl₂/MeOH (20:1) to afford tert-butyl N-cyclobutyl-N-[(3S)-1-{6-[5-fluoro-2-(methoxymethoxy)-4-(4-methylimidazol-1-yl)phenyl]pyridazin-3-yl}pyrrolidin-3-yl]carbamate (50 mg, 62%) as an oil. LCMS (ES, m/z): 553 [M+H]⁺.

### Synthesis of Compound 227

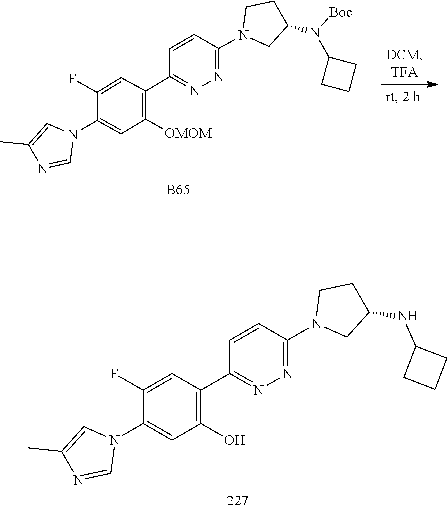

A mixture of tert-butyl N-cyclobutyl-N-[(3S)-1-{6-[5-fluoro-2-(methoxymethoxy)-4-(4-methylimidazol-1-yl)phenyl]pyridazin-3-yl}pyrrolidin-3-yl]carbamate (50 mg, 0.090 mmol, 1 equiv), DCM (1 mL) and TFA (1 mL) was stirred for 2 h at room temperature. The resulting mixture was concentrated under reduced pressure to give a residue. The residue was purified by reverse phase flash chromatography (Condition 1, Gradient 1) to afford 2-{6-[(3S)-3-(cyclobutylamino)pyrrolidin-1-yl]pyridazin-3-yl}-4-fluoro-5-(4-methylimidazol-1-yl)phenol (10 mg, 27%) as a solid. LCMS (ES, m/z): 409 [M+H]⁺. ¹H NMR (400 MHz, Methanol-d₄) δ 8.13 (d, J=9.8 Hz, 1H), 7.96 (t, J=1.7 Hz, 1H), 7.82 (d, J=12.6 Hz, 1H), 7.27-7.22 (m, 1H), 7.19 (d, J=9.7 Hz, 1H), 7.12 (d, J=6.8 Hz, 1H), 3.81 (dd, J=10.8, 6.4 Hz, 1H), 3.75 (d, J=7.7 Hz, 1H), 3.59 (q, J=7.4, 6.1 Hz, 2H), 3.49-3.38 (m, 2H), 2.39-2.26 (m, 6H), 2.07-1.84 (m, 3H), 1.76 (ddd, J=13.9, 11.6, 6.9 Hz, 2H).

### Example 24: Synthesis of Compound 145

### Synthesis of Intermediate B66

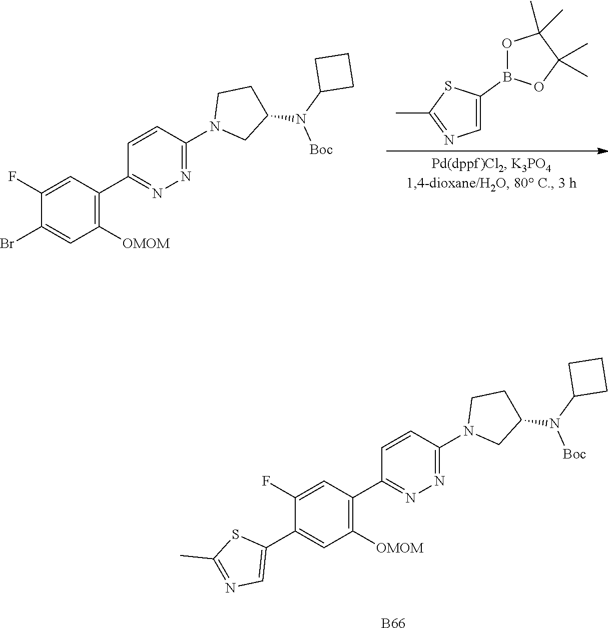

A mixture of tert-butyl N-[(3 S)-i-{6-[4-bromo-5-fluoro-2-(methoxymethoxy)phenyl]pyridazin-3-yl}pyrrolidin-3-yl]-N-cyclobutylcarbamate (80 mg, 0.145 mmol, 1.0 equiv), dioxane/H₂O (1 mL/0.2 mL), 2-methyl-5-(4,4,5,5-tetramethyl-1,3,2-dioxaborolan-2-yl)-1,3-thiazole (39.19 mg, 0.174 mmol, 1.2 equiv), K₃PO₄ (61.59 mg, 0.290 mmol, 2.0 equiv), and Pd(dpp)Cl₂ (11.82 mg, 0.014 mmol, 0.1 equiv) was stirred for 4 h at 80° C. under nitrogen atmosphere. The mixture was concentrate under reduced pressure to give a residue. The residue was purified by silica gel column chromatography, eluted with PE/EA (1:3) to afford tert-butyl N-cyclobutyl-N-[(3S)-i-{6-[5-fluoro-2-(methoxymethoxy)-4-(2-methyl-1,3-thiazol-5-yl)phenyl]pyridazin-3-yl}pyrrolidin-3-yl]carbamate (50 mg, 61%) as an oil. LCMS (ES, m/z): 570 [M+H]n.

### Synthesis of Compound 145

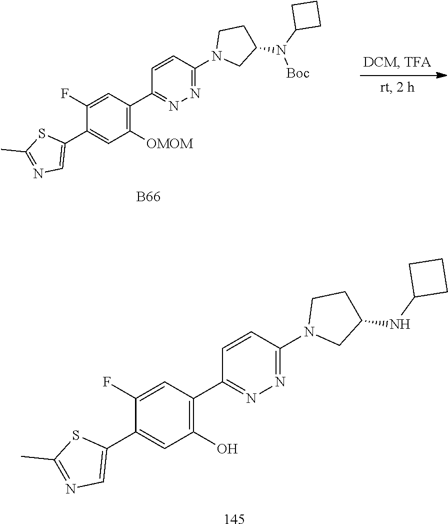

A mixture of tert-butyl N-cyclobutyl-N-[(3S)-1-{6-[5-fluoro-2-(methoxymethoxy)-4-(2-methyl-1,3-thiazol-5-yl)phenyl]pyridazin-3-yl}pyrrolidin-3-yl]carbamate (50 mg, 0.088 mmol, 1 equiv), DCM (1 mL) and TFA (1 mL) was stirred for 3 h at room temperature. The mixture was concentrate under reduced pressure to give a residue. The residue was purified by Prep-HPLC (Condition 1, Gradient 1) to afford 2-{6-[(3S)-3-(cyclobutylamino)pyrrolidin-1-yl]pyridazin-3-yl}-4-fluoro-5-(2-methyl-1,3-thiazol-5-yl)phenol (2 mg, 5%) as a solid. LCMS (ES, m/z): 426 [M+H]⁺. ¹H NMR (400 MHz, Methanol-d₄) δ 8.11 (d, J=9.8 Hz, 1H), 8.04 (s, 1H), 7.69 (d, J=12.5 Hz, 1H), 7.22 (d, J=6.6 Hz, 1H), 7.16 (d, J=9.8 Hz, 1H), 3.83-3.71 (m, 2H), 3.59 (d, J=7.6 Hz, 1H), 3.53 (dd, J=13.0, 7.0 Hz, 2H), 3.39 (t, J=7.9 Hz, 1H), 2.76 (s, 3H), 2.30 (dd, J=12.3, 6.4 Hz, 3H), 1.97 (dd, J=13.0, 7.0 Hz, 1H), 1.86 (q, J=9.5 Hz, 2H), 1.74 (s, 2H).

### Example 25: Synthesis of Compound 146

### Synthesis of Intermediate B67

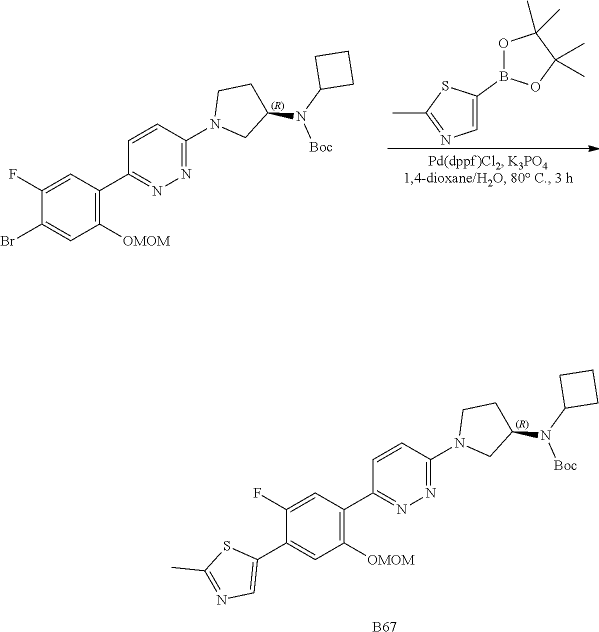

A mixture of tert-butyl N-[(3R)-1-{6-[4-bromo-5-fluoro-2-(methoxymethoxy)phenyl]pyridazin-3-yl}pyrrolidin-3-yl]-N-cyclobutylcarbamate (80 mg, 0.145 mmol, 1.0 equiv), dioxane/H₂O (1 mL/0.2 mL), 2-methyl-5-(4,4,5,5-tetramethyl-1,3,2-dioxaborolan-2-yl)-1,3-thiazole (39.19 mg, 0.174 mmol, 1.2 equiv), K₃PO₄ (61.59 mg, 0.290 mmol, 2.0 equiv) and Pd(dppf)Cl₂ (11.82 mg, 0.014 mmol, 0.1 equiv) was stirred for 4 h at 80° C. under nitrogen atmosphere. The mixture was concentrated under reduced pressure to give a residue. The residue was purified by silica gel column chromatography, eluted with PE/EA (1:1) to afford tert-butyl N-cyclobutyl-N-[(3R)-1-{6-[5-fluoro-2-(methoxymethoxy)-4-(2-methyl-1,3-thiazol-5-yl)phenyl]pyridazin-3-yl}pyrrolidin-3-yl]carbamate (50 mg, 61%) as an oil. LCMS (ES, m/z): 570 [M+H]⁺.

### Synthesis of Compound 146

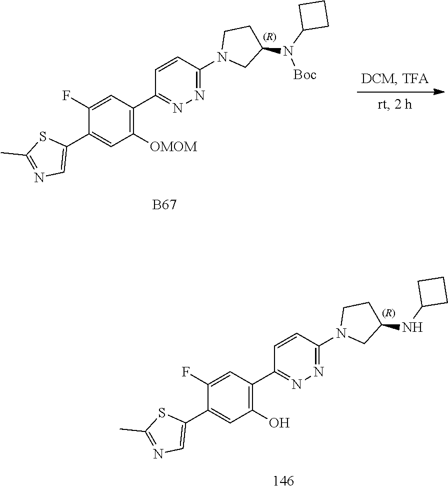

A mixture of tert-butyl N-cyclobutyl-N-[(3R)-1-{6-[5-fluoro-2-(methoxymethoxy)-4-(2-methyl-1,3-thiazol-5-yl)phenyl]pyridazin-3-yl}pyrrolidin-3-yl]carbamate (50 mg, 0.088 mmol, 1 equiv), DCM (1 mL) and TFA (1 mL) was stirred for 2 h at room temperature. The mixture was concentrated under reduced pressure to give a residue. The residue was purified by Prep-HPLC (Condition 1, Gradient 1) to afford 2-{6-[(3R)-3-(cyclobutylamino)pyrrolidin-1-yl]pyridazin-3-yl}-4-fluoro-5-(2-methyl-1,3-thiazol-5-yl)phenol (4 mg) as a solid. LCMS (ES, m/z): 426 [M+H]⁺. ¹H NMR (400 MHz, Chloroform-d) δ 13.46 (s, 1H), 8.04 (s, 1H), 7.75 (d, J=9.7 Hz, 1H), 7.36 (d, J=12.0 Hz, 1H), 7.24 (d, J=6.7 Hz, 1H), 6.87 (d, J=9.8 Hz, 1H), 3.79 (s, 2H), 3.59 (t, J=5.6 Hz, 2H), 3.41 (s, 2H), 2.77 (s, 3H), 2.31 (s, 3H), 1.96 (s, 1H), 1.75 (s, 4H).

### Example 26: Synthesis of Compound 228

### Synthesis of Intermediate B68

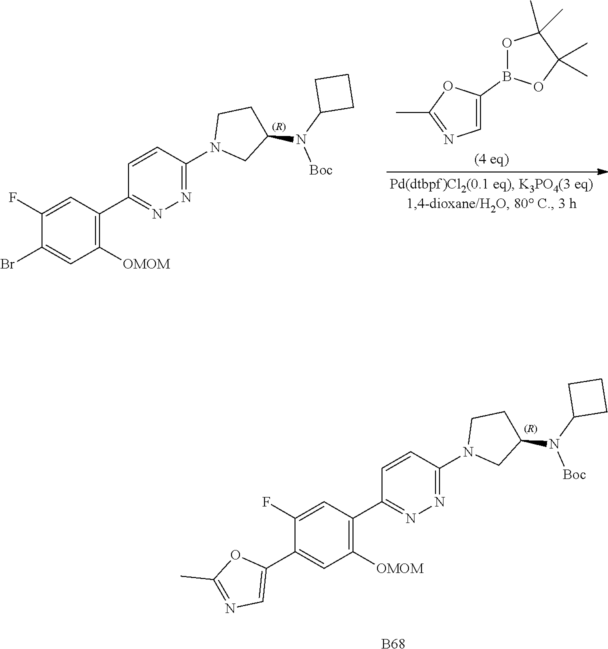

A solution of tert-butyl N-[(3R)-1-{6-[4-bromo-5-fluoro-2-(methoxymethoxy)phenyl]pyridazin-3-yl}pyrrolidin-3-yl]-N-cyclobutylcarbamate (70 mg, 0.127 mmol, 1 equiv) in 1,4-dioxane (2 mL) and H₂O (0.5 mL) was treated with K₃PO₄ (80.83 mg, 0.381 mmol, 3 equiv) and Pd(DtBPF)Cl₂ (8.27 mg, 0.013 mmol, 0.1 equiv). The reaction mixture was stirred for 2 h at 80° C. under nitrogen atmosphere. To the resulting mixture was added 2-methyl-5-(4,4,5,5-tetramethyl-1,3,2-dioxaborolan-2-yl)-1,3-oxazole (39.80 mg, 0.191 mmol, 1.5 equiv) dropwise at 80° C. The mixture was cooled to room temperature, then quenched with H₂O at room temperature. The resulting mixture was extracted with ethyl acetate (3×10 mL). The organic layers were combined, washed with NaCl Solution (2×10 mL), dried over anhydrous Na₂SO₄, and filtered. After filtration, the filtrate was concentrated under reduced pressure to give a residue. The residue was purified by silica gel column chromatography, eluted with PE:EA (1:1) to afford tert-butyl N-cyclobutyl-N-[(3R)-1-{6-[5-fluoro-2-(methoxymethoxy)-4-(2-methyl-1,3-oxazol-5-yl)phenyl]pyridazin-3-yl}pyrrolidin-3-yl]carbamate (56 mg, 80%) as a solid. LCMS (ES, m/z): 446 [M+H]⁺.

### Synthesis of Compound 228

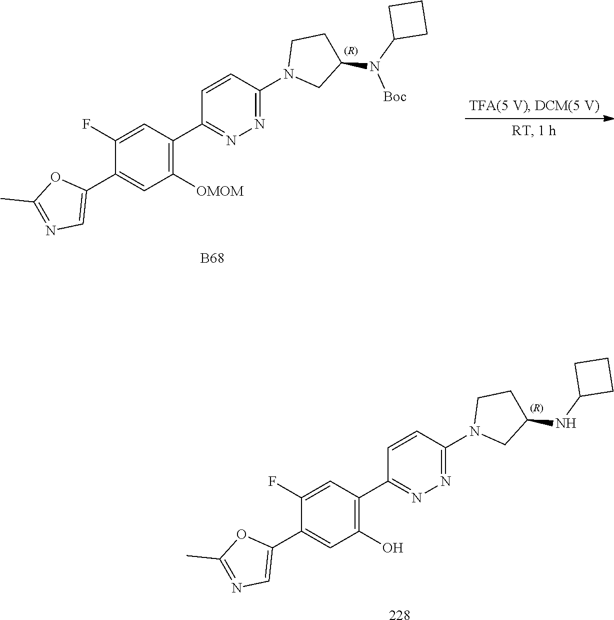

To a stirred solution of tert-butyl N-cyclobutyl-N-[(3R)-1-{6-[5-fluoro-2-(methoxymethoxy)-4-(2-methyl-1,3-oxazol-5-yl)phenyl]pyridazin-3-yl}pyrrolidin-3-yl]carbamate (56 mg, 0.101 mmol, 1 equiv) in DCM (2 mL) was added TFA (2 mL) dropwise at room temperature. The mixture was stirred for 1 h at room temperature, then concentrated under reduced pressure to give a residue. The residue was purified by reverse phase flash chromatography (Condition 6, Gradient 1) to afford 2-{6-[(3R)-3-(cyclobutylamino)pyrrolidin-1-yl]pyridazin-3-yl}-4-fluoro-5-(2-methyl-1,3-oxazol-5-yl)phenol (12.7 mg, 30%) as a solid. LCMS (ES, m/z): 410 [M+H]⁺. ¹H NMR (400 MHz, DMSO-d₆) δ 13.69 (s, 1H), 8.26 (d, J=9.9 Hz, 1H), 7.94 (d, J=12.5 Hz, 1H), 7.43 (d, J=3.6 Hz, 1H), 7.22-7.12 (m, 2H), 3.64 (dd, J=10.4, 6.1 Hz, 2H), 3.50 (d, J=8.7 Hz, 1H), 3.42-3.34 (m, 1H), 3.25 (t, J=7.7 Hz, 2H), 2.67 (s, 3H), 2.18-2.02 (m, 3H), 1.82 (dq, J=13.2, 6.8 Hz, 1H), 1.72-1.66 (m, 2H), 1.66-1.49 (m, 2H).

### Example 27: Synthesis of Compound 229

### Synthesis of Intermediate B69

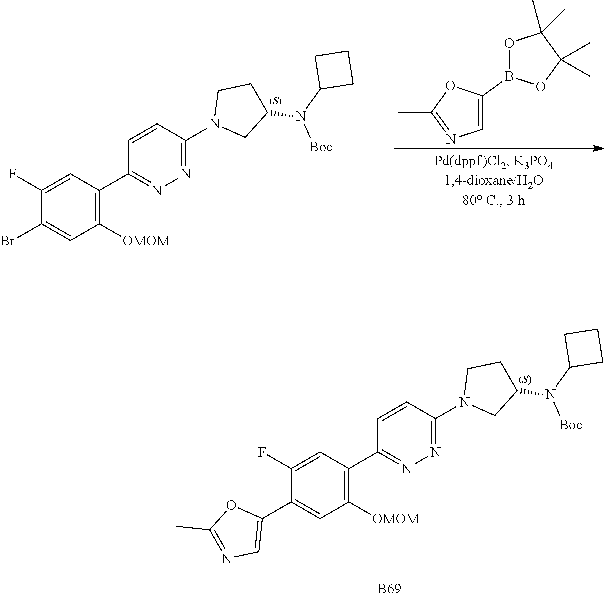

A solution of tert-butyl N-[(3S)-1-{6-[4-bromo-5-fluoro-2-(methoxymethoxy) phenyl]pyridazin-3-yl}pyrrolidin-3-yl]-N-cyclobutylcarbamate (60 mg, 0.109 mmol, 1 equiv) in dioxane (2 mL) was treated with 2-methyl-5-(4,4,5,5-tetramethyl-1,3,2-dioxaborolan-2-yl)-1,3-oxazole (22.75 mg, 0.109 mmol, 1 equiv) and K₃PO₄ (69.29 mg, 0.327 mmol, 3 equiv) in H₂O (0.5 mL). The reaction mixture was stirred for 3 h at 100° C. under nitrogen atmosphere. The resulting mixture was diluted with water (50 mL) and extracted with ethyl acetate (3×20 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with PE/EA (5:1) to afford tert-butyl N-cyclobutyl-N-[(3S)-1-{6-[5-fluoro-2-(methoxymethoxy)-4-(2-methyl-1,3-oxazol-5-yl) phenyl]pyridazin-3-yl}pyrrolidin-3-yl]carbamate (45 mg, 75%). LCMS (ES, m/z):554 [M+H]⁺.

### Synthesis of Compound 229

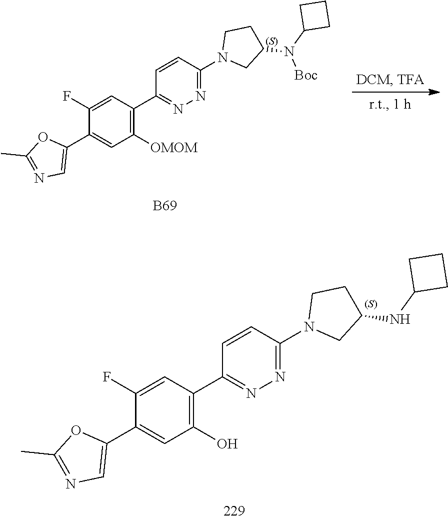

A solution of tert-butyl N-cyclobutyl-N-[(3S)-1-{6-[5-fluoro-2-(methoxymethoxy)-4-(2-methyl-1,3-oxazol-5-yl) phenyl]pyridazin-3-yl}pyrrolidin-3-yl]carbamate (60 mg, 0.108 mmol, 1 equiv) in CH₂Cl₂ was treated with TFA (0.19 mL) for 1 h at room temperature. The resulting mixture was concentrated under reduced pressure to give a residue. The residue was basified to pH 8 with TEA, then concentrated under reduced pressure and extracted with ethyl acetate (3×20 mL). The organic layers were combined, washed with water (2×10 mL), dried over anhydrous Na₂SO₄, and filtered. After filtration, the filtrate was concentrated under reduced pressure to afford 2-{6-[(3S)-3-(cyclobutylamino) pyrrolidin-1-yl]pyridazin-3-yl}-4-fluoro-5-(2-methyl-1,3-oxazol-5-yl) phenol (12.4 mg, 28%) as a solid. LCMS (ES, m/z):410 [M+H]⁺. H NMR (400 MHz, DMSO-d₆) δ 13.70 (s, 1H), 8.26 (d, J=9.8 Hz, 1H), 7.94 (d, J=12.5 Hz, 1H), 7.43 (d, J=3.6 Hz, 1H), 7.22-7.11 (m, 2H), 3.63 (dd, J=10.5, 6.0 Hz, 2H), 3.49 (d, J=7.8 Hz, 1H), 3.38 (d, J=11.2 Hz, 1H), 3.24 (p, J=7.5 Hz, 2H), 2.52 (s, 3H), 2.19-2.02 (m, 4H), 1.82 (dq, J 13.0, 6.6 Hz, 1H), 1.76-1.67 (m, 1H), 1.71-1.57 (m, 2H), 1.61-1.48 (m, 1H).

### Example 28: Synthesis of Compound 120

### Synthesis of Intermediate B70

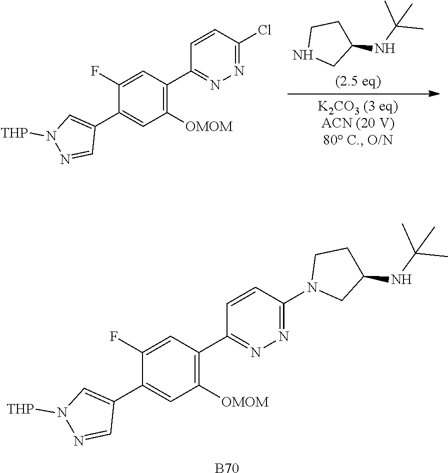

A mixture of 3-chloro-6-[5-fluoro-2-(methoxymethoxy)-4-[1-(oxan-2-yl)pyrazol-4-yl]phenyl]pyridazine (60 mg, 0.143 mmol, 1 equiv), (3R)—N-tert-butylpyrrolidin-3-amine (50.94 mg, 0.357 mmol, 2.5 equiv) and K₂CO₃ (59.39 mg, 0.429 mmol, 3 equiv) in acetonitrile was stirred for 2 days at 80° C. under nitrogen atmosphere. The resulting mixture was extracted with CH₂Cl₂ (5×10 mL). The organic layers were combined, dried over anhydrous Na₂SO₄, and filtered. After filtration, the filtrate was concentrated under reduced pressure to afford (3R)—N-tert-butyl-1-{6-[5-fluoro-2-(methoxymethoxy)-4-[1-(oxan-2-yl)pyrazol-4-yl]phenyl]pyridazin-3-yl}pyrrolidin-3-amine (75 mg, 100%) as a solid.

### Synthesis of Intermediate B71

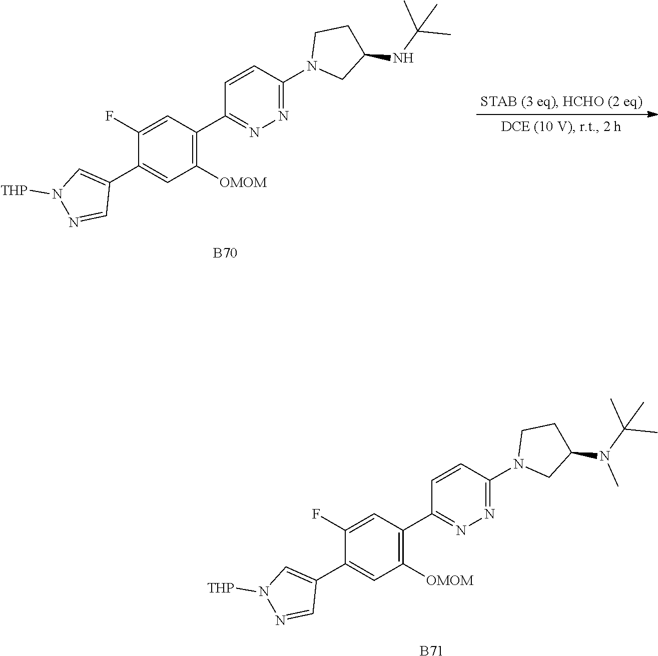

A mixture of (3R)—N-tert-butyl-1-{6-[5-fluoro-2-(methoxymethoxy)-4-[1-(oxan-2-yl)pyrazol-4-yl]phenyl]pyridazin-3-yl}pyrrolidin-3-amine (65 mg, 0.124 mmol, 1 equiv) and HCHO (7.44 mg, 0.248 mmol, 2 equiv) in DCE (6.5 mL) was stirred for 10 min at room temperature. To the resulting mixture was added STAB (78.77 mg, 0.372 mmol, 3 equiv) in portions at room temperature. The resulting mixture was extracted with CH₂Cl₂ (5×5 mL). The organic layers were combined, dried over anhydrous Na₂SO₄, and filtered. After filtration, the filtrate was concentrated under reduced pressure to afford (3R)—N-tert-butyl-1-{6-[5-fluoro-2-(methoxymethoxy)-4-[1-(oxan-2-yl)pyrazol-4-yl]phenyl]pyridazin-3-yl}-N-methylpyrrolidin-3-amine (110 mg) as a solid.

### Synthesis of Compound 120

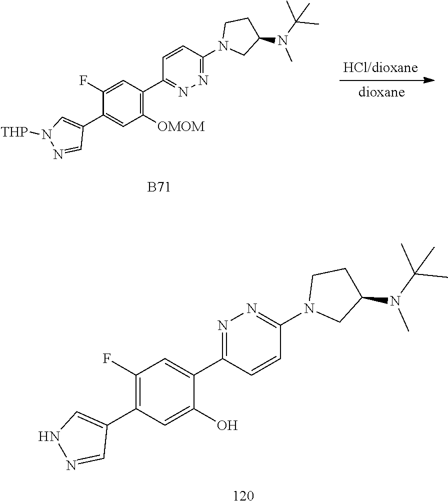

A mixture of (3R)—N-tert-butyl-1-{6-[5-fluoro-2-(methoxymethoxy)-4-[1-(oxan-2-yl)pyrazol-4-yl]phenyl]pyridazin-3-yl}-N-methylpyrrolidin-3-amine (100 mg, 0.186 mmol, 1 equiv) and HCl (gas) in 1,4-dioxane (4 mol/L, 2 mL) was stirred for 6 h at room temperature. The resulting mixture was neutralized to pH 7 with saturated NaHCO₃ (aq.), then concentrated under reduced pressure to give a residue. The residue was purified by Chiral-Prep-HPLC (Condition 7, Gradient 1), followed by Chiral-Prep-HPLC (Condition 8, Gradient 1) to afford 2-{6-[(3R)-3-[tert-butyl(methyl)amino]pyrrolidin-1-yl]pyridazin-3-yl}-4-fluoro-5-(1H-pyrazol-4-yl)phenol (6.47 mg, 8%) as a solid. LCMS (ES, m/z): 411 [M+H]⁺. ¹H NMR (400 MHz, DMSO-d₆) δ 13.66 (s, 1H), 13.12 (s, 1H), 8.24 (d, J=9.9 Hz, 2H), 7.99 (s, 1H), 7.82 (d, J=12.7 Hz, 1H), 7.29 (d, J=7.0 Hz, 1H), 7.19 (d, J=9.7 Hz, 1H), 4.05-3.97 (m, J=9.0 Hz, 1H), 3.70 (t, J=9.6 Hz, 1H), 3.48 (d, J=9.8 Hz, 1H), 3.43-3.36 (m, 1H), 3.30 (s, 1H), 2.22 (s, 3H), 2.05 (t, J=10.2 Hz, 1H), 1.92 (d, J=8.2 Hz, 1H), 1.10 (s, 9H).

### Example 29: Synthesis of Compound 119

### Synthesis of Intermediate B72

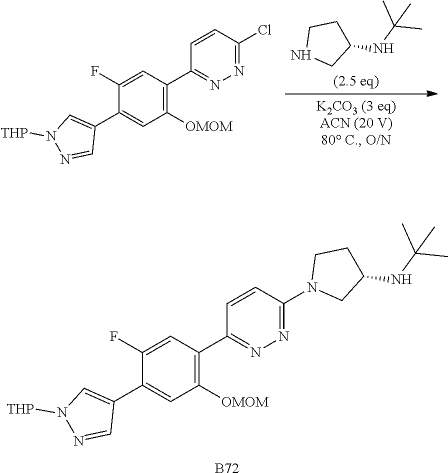

A mixture of 3-chloro-6-[5-fluoro-2-(methoxymethoxy)-4-[1-(oxan-2-yl)pyrazol-4-yl]phenyl]pyridazine (60 mg, 0.143 mmol, 1 equiv), (3S)—N-tert-butylpyrrolidin-3-amine (50.94 mg, 0.357 mmol, 2.5 equiv), and K₂CO₃ (59.39 mg, 0.429 mmol, 3 equiv) in acetonitrile was stirred for 2 days at 80° C. under nitrogen atmosphere. The resulting mixture was extracted with CH₂Cl₂ (5×10 mL). The organic layers were combined, dried over anhydrous Na₂SO₄, and filtered. The filtrate was concentrated under reduced pressure to afford the crude product (3S)—N-(tert-butyl)-1-(6-(5-fluoro-2-(methoxymethoxy)-4-(1-(tetrahydro-2H-pyran-2-yl)-1H-pyrazol-4-yl)phenyl)pyridazin-3-yl)pyrrolidin-3-amine as a solid.

### Synthesis of Intermediate B73

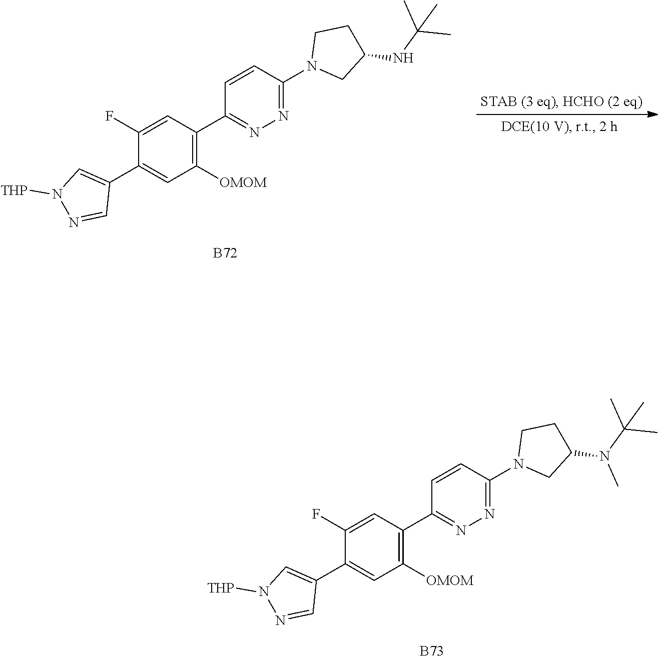

A mixture of (3s)-N-tert-butyl-1-{6-[5-fluoro-2-(methoxymethoxy)-4-[1-(oxan-2-yl)pyrazol-4-yl]phenyl]pyridazin-3-yl}pyrrolidin-3-amine (10 mg, 0.019 mmol, 1 equiv) and HCHO (1.14 mg, 0.038 mmol, 2 equiv) in DCE(1 mL) was stirred for 10 min at room temperature. To the reaction mixture was added STAB (12.12 mg, 0.057 mmol, 3 equiv) in portions at room temperature. The resulting mixture was extracted with CH₂Cl₂ (3×20 mL). The organic layers were combined, washed with saturated aqueous NaCl (50 mL), dried over anhydrous Na₂SO₄, and filtered. The filtrate was concentrated under reduced pressure to afford the crude product (3S)—N-tert-butyl-1-{6-[5-fluoro-2-(methoxymethoxy)-4-[1-(oxan-2-yl)pyrazol-4-yl]phenyl]pyridazin-3-yl}-N-methylpyrrolidin-3-amine as a solid.

### Synthesis of Compound 119

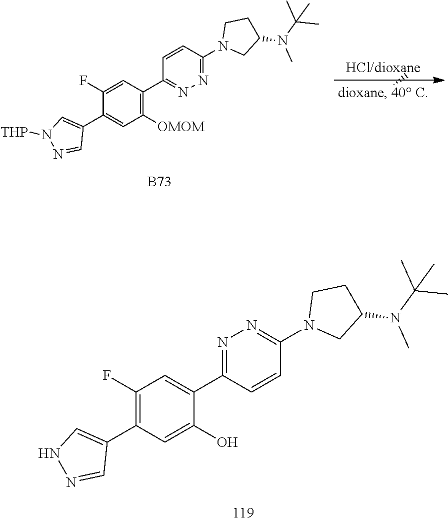

A mixture of(3S)—N-tert-butyl-1-{6-[5-fluoro-2-(methoxymethoxy)-4-[1-(oxan-2-yl)pyrazol-4-yl]phenyl]pyridazin-3-yl}-N-methylpyrrolidin-3-amine (60 mg, 0.186 mmol, 1 equiv) and HCl (gas) in 1,4-dioxane (4M/L, 40 mL) at 40° C. The resulting mixture was stirred for 4 h at 40° C., then neutralized to pH 7 with saturated NaHCO₃ (aq.) and concentrated under reduced pressure to give a residue. The residue was purified by Chiral-Prep-HPLC (Condition 7, Gradient 1) to afford 2-{6-[(3S)-3-[tert-butyl(methyl)amino]pyrrolidin-1-yl]pyridazin-3-yl}-4-fluoro-5-(1H-pyrazol-4-yl)phenol (8.6 mg, 8%) as a solid. LCMS (ES, m/z): 411 [M+H]⁺. ¹H NMR (400 MHz, DMSO-d₆) δ 13.62 (s, 1H), 13.10 (s, 1H), 8.22 (d, J=9.8 Hz, 2H), 7.98 (s, 1H), 7.81 (d, J=12.7 Hz, 1H), 7.28 (d, J=7.0 Hz, 1H), 7.18 (d, J=9.7 Hz, 1H), 4.00-3.91 (m, 1H), 3.70 (t, J=9.7 Hz, 1H), 3.49 (t, J=9.4 Hz, 1H), 3.44-3.35 (m, 1H), 3.27 (s, 1H), 2.22 (s, 3H), 2.05 (t, J=10.4, 9.8 Hz, 1H), 1.93 (s, 1H), 1.10 (s, 9H).

### Example 30: Synthesis of Compound 230

### Synthesis of Intermediate B74

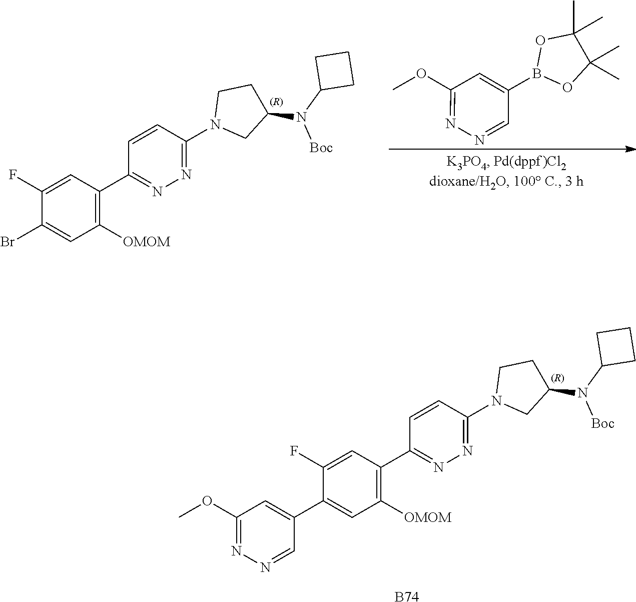

To a stirred mixture of tert-butyl N-[(3R)-1-{6-[4-bromo-5-fluoro-2-(methoxymethoxy)phenyl]pyridazin-3-yl}pyrrolidin-3-yl]-N-cyclobutylcarbamate (80 mg, 0.145 mmol, 1 equiv) and 3-methoxy-5-(4,4,5,5-tetramethyl-1,3,2-dioxaborolan-2-yl)pyridazine (41.10 mg, 0.174 mmol, 1.2 equiv) in a mixture of dioxane (1 mL) and H₂O (0.2 mL) was added K₃PO₄ (92.38 mg, 0.435 mmol, 3 equiv) and Pd(dppf)Cl₂ (10.61 mg, 0.014 mmol, 0.1 equiv) at room temperature under nitrogen atmosphere. The resulting mixture was stirred for 3 h at 100° C. under nitrogen atmosphere, then concentrated under reduced pressure to give a residue. The residue was purified by silica gel column chromatography, eluted with CH₂Cl₂/MeOH (20:1) to afford tert-butyl N-cyclobutyl-N-[(3R)-1-{6-[5-fluoro-2-(methoxymethoxy)-4-(6-methoxypyridazin-4-yl)phenyl]pyridazin-3-yl}pyrrolidin-3-yl]carbamate (60 mg, 71%) as a solid. LCMS (ES, m/z): 581 [M+H]⁺.

### Synthesis of Compound 230

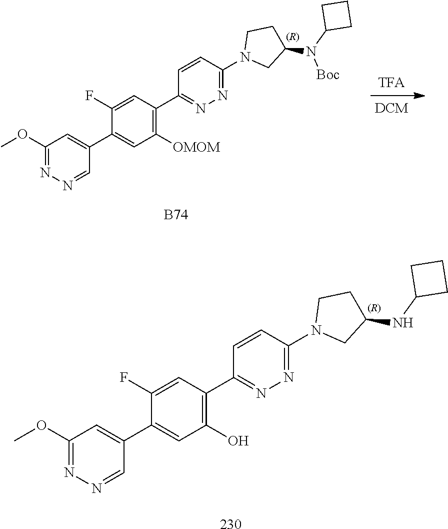

To a stirred solution of tert-butyl N-cyclobutyl-N-[(3R)-1-{6-[5-fluoro-2-(methoxymethoxy)-4-(6-methoxypyridazin-4-yl)phenyl]pyridazin-3-yl}pyrrolidin-3-yl]carbamate (60 mg, 0.103 mmol, 1 equiv) in DCM (1 mL) was added TFA (1 mL) at room temperature. The resulting mixture was stirred for 1 h at room temperature, then concentrated under reduced pressure to give a residue. The residue was purified by Prep-HPLC (Condition 5, Gradient 1) to afford 2-{6-[(3R)-3-(cyclobutylamino)pyrrolidin-1-yl]pyridazin-3-yl}-4-fluoro-5-(6-methoxypyridazin-4-yl)phenol (20.2 mg, 45%) as a solid. LCMS (ES, m/z): 437 [M+H]⁺. ¹H NMR (400 MHz, Methanol-d₄) δ 9.09 (t, J=1.8 Hz, 1H), 8.13 (d, J=9.8 Hz, 1H), 7.74 (d, J=12.3 Hz, 1H), 7.41 (t, J=1.3 Hz, 1H), 7.23 (d, J=6.8 Hz, 1H), 7.16 (d, J=9.7 Hz, 1H), 4.17 (s, 3H), 3.83-3.69 (m, 2H), 3.54 (ddt, J=18.4, 12.5, 6.8 Hz, 2H), 3.39 (p, J=7.8 Hz, 2H), 2.29 (ddt, J=9.7, 7.0, 4.8 Hz, 3H), 2.03-1.92 (m, 1H), 1.86 (ddd, J=12.9, 10.2, 8.1 Hz, 2H), 1.74 (dtd, J=13.7, 6.5, 3.2 Hz, 2H).

### Example 31: Synthesis of Compound 231

### Synthesis of Intermediate B75

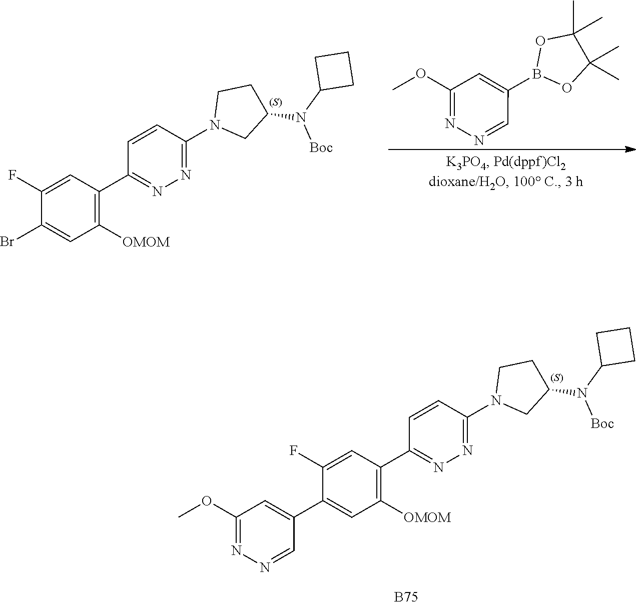

To a stirred mixture of tert-butyl N-[(3S)-1-{6-[4-bromo-5-fluoro-2-(methoxymethoxy)phenyl]pyridazin-3-yl}pyrrolidin-3-yl]-N-cyclobutylcarbamate (80 mg, 0.145 mmol, 1 equiv) and 3-methoxy-5-(4,4,5,5-tetramethyl-1,3,2-dioxaborolan-2-yl)pyridazine (41.10 mg, 0.174 mmol, 1.2 equiv) in a mixture of dioxane (1 mL) and H₂O (0.2 mL) was added K₃PO₄ (92.38 mg, 0.435 mmol, 3 equiv) and Pd(dppf)Cl₂ (10.61 mg, 0.014 mmol, 0.1 equiv) at room temperature under nitrogen atmosphere. The resulting mixture was stirred for 3 h at 100° C. under nitrogen atmosphere, then concentrated under reduced pressure to give a residue. The residue was purified by silica gel column chromatography, eluted with CH₂Cl₂/MeOH (20:1) to afford tert-butyl N-cyclobutyl-N-[(3S)-1-{6-[5-fluoro-2-(methoxymethoxy)-4-(6-methoxypyridazin-4-yl)phenyl]pyridazin-3-yl}pyrrolidin-3-yl]carbamate (60 mg, 71%) as a solid. LCMS (ES, m/z): 581 [M+H]⁺.

### Synthesis of Compound 231

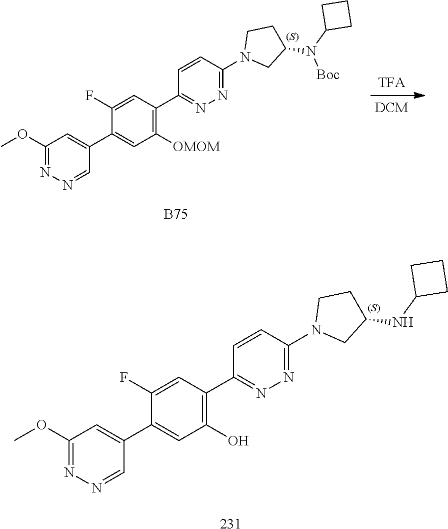

To a stirred solution of tert-butyl N-cyclobutyl-N-[(3S)-1-{6-[5-fluoro-2-(methoxymethoxy)-4-(6-methoxypyridazin-4-yl)phenyl]pyridazin-3-yl}pyrrolidin-3-yl]carbamate (60 mg, 0.103 mmol, 1 equiv) in DCM (1 mL) was added TFA (1 mL) at room temperature. The resulting mixture was stirred for 1 h at room temperature, then concentrated under reduced pressure to give a residue. The residue was purified by Prep-HPLC (Condition 5, Gradient 1) to afford 2-{6-[(3S)-3-(cyclobutylamino)pyrrolidin-1-yl]pyridazin-3-yl}-4-fluoro-5-(6-methoxypyridazin-4-yl)phenol (22.5 mg, 50%) as a solid. LCMS (ES, m/z): 437 [M+H]⁺. ¹H NMR (400 MHz, Methanol-d₄) δ 9.09 (t, J=1.8 Hz, 1H), 8.14 (d, J=9.8 Hz, 1H), 7.75 (d, J=12.3 Hz, 1H), 7.41 (d, J=1.5 Hz, 1H), 7.23 (d, J=6.8 Hz, 1H), 7.17 (d, J=9.8 Hz, 1H), 4.17 (s, 3H), 3.76 (tt, J=13.1, 6.5 Hz, 2H), 3.54 (ddt, J=18.4, 12.5, 6.8 Hz, 2H), 3.39 (p, J=7.5 Hz, 2H), 2.30 (dq, J=10.8, 6.6 Hz, 3H), 2.03-1.91 (m, 1H), 1.86 (dtd, J=10.2, 8.3, 7.9, 2.0 Hz, 2H), 1.74 (ddt, J=14.0, 10.6, 6.2 Hz, 2H).

### Example 32: Synthesis of Compound 100

### Synthesis of Intermediate B76

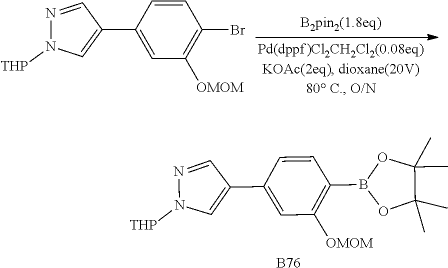

A flask containing a mixture of 4-(4-bromo-3-(methoxymethoxy)phenyl)-1-(tetrahydro-2H-pyran-2-yl)-1H-pyrazole (3.25 g, 8.88 mmol, 1.00 equiv), bis(pinacolato)diboron (4.06 g, 15.98 mmol, 1.80 equiv), potassium acetate (1.74 g, 17.76 mmol, 2.00 equiv), Pd(dppf)Cl₂ CH₂Cl₂ (0.72 g, 0.888 mmol, 0.10 equiv) and dioxane (65 mL) was evacuated and flushed three times with nitrogen. The resulting solution was stirred for 16 h at 80° C. Solids were removed by filtration, and the reaction mixture was quenched with water (100 mL). The resulting solution was extracted with ethyl acetate (3×100 mL). The organic layers were combined, washed with saturated aqueous NaCl (1×300 mL), dried over anhydrous sodium sulfate, and filtered. The filtrate was concentrated under vacuum to give a residue. The residue was applied onto a silica gel column with ethyl acetate/petroleum ether to afford 4-(3-(methoxymethoxy)-4-(4,4,5,5-tetramethyl-1,3,2-dioxaborolan-2-yl)phenyl)-1-(tetrahydro-2H-pyran-2-yl)-1H-pyrazole (1.7 g, 46%) an oil. LCMS (ES, m/z): 415 [M+H]⁺.

### Synthesis of Intermediate B77

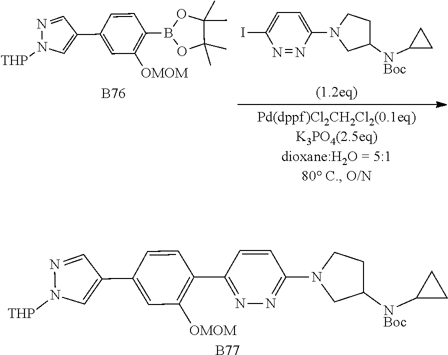

A flask containing a mixture of 4-(3-(methoxymethoxy)-4-(4,4,5,5-tetramethyl-1,3,2-dioxaborolan-2-yl) phenyl)-1-(tetrahydro-2H-pyran-2-yl)-1H-pyrazole (50.0 mg, 0.12 mmol, 1.00 equiv), tert-butyl cyclopropyl(1-(6-iodopyridazin-3-yl)pyrrolidin-3-yl)carbamate (62.3 mg, 0.14 mmol, 1.20 equiv), dioxane (2 mL), K₃PO₄ (64.0 mg, 0.30 mmol, 2.50 equiv), H₂O (0.4 mL), and Pd(dppf)Cl₂ CH₂Cl₂ (9.8 mg, 0.012 mmol, 0.10 equiv) was evacuated and flushed three times with nitrogen. The resulting solution was stirred for 16 h at 80° C., then quenched with water (20 mL) and extracted with ethyl acetate (3×20 mL). The organic layers were combined, washed with saturated aqueous NaCl (1×50 mL), dried over anhydrous sodium sulfate, and filtered. The filtrate was concentrated under vacuum to give a residue. The residue was purified by reverse phase flash chromatography to afford tert-butyl cyclopropyl(1-(6-(2-hydroxy-4-(1-(tetrahydro-2H-pyran-2-yl)-1H-pyrazol-4-yl)phenyl)pyridazin-3-yl)pyrrolidin-3-yl)carbamate (15.0 mg, 21.0%) as a solid. LCMS (ES, m/z): 591 [M+H]⁺.

### Synthesis of Compound 100

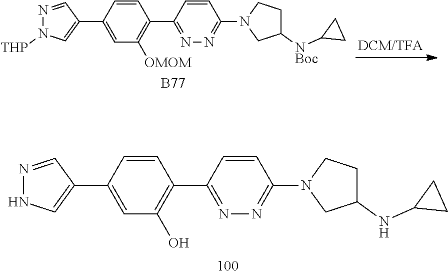

A mixture of tert-butyl cyclopropyl(1-(6-(2-hydroxy-4-(1-(tetrahydro-2H-pyran-2-yl)-1H-pyrazol-4-yl)phenyl)pyridazin-3-yl)pyrrolidin-3-yl)carbamate (15.0 mg, 0.025 mmol, 1.00 equiv), DCM (1.0 mL), and TFA (0.2 mL) was stirred for 4 h at room temperature. The resulting mixture was concentrated under vacuum to give a residue. The residue was purified by Prep-HPLC (Condition 9, Gradient 2) to afford 2-(6-(3-(cyclopropylamino)pyrrolidin-1-yl)pyridazin-3-yl)-5-(1H-pyrazol-4-yl)phenol (4.2 mg, 45%) as a solid. LCMS (ES, m/z): 363 [M+H]^{+ 1}H-NMR (400 MHz, DMSO-d₆) δ 13.92 (s, 1H), 12.97 (s, 1H), 8.26 (s, 1H), 8.21 (d, J=9.9 Hz, 1H), 7.97 (s, 1H), 7.83 (d, J=8.3 Hz, 1H), 7.22-7.11 (m, 3H), 3.69 (dd, J=10.7, 5.9 Hz, 1H), 3.65-3.56 (m, 1H), 3.56-3.46 (m, 2H), 3.41-3.34 (m, 1H), 2.20-2.08 (m, 2H), 1.93 (dq, J=12.8, 6.4 Hz, 1H), 0.48-0.35 (m, 2H), 0.33-0.19 (m, 2H).

### Example 33: Synthesis of Compound 101

### Synthesis of Intermediate B78

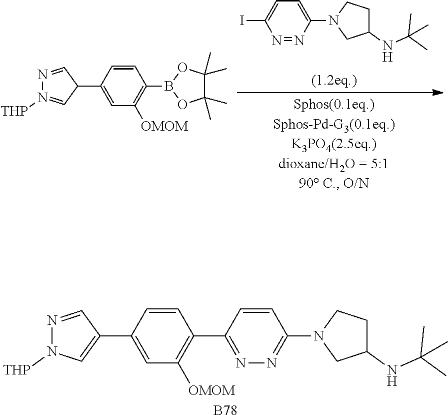

A flask containing a mixture of 4-(3-(methoxymethoxy)-4-(4,4,5,5-tetramethyl-1,3,2-dioxaborolan-2-yl) phenyl)-1-(tetrahydro-2H-pyran-2-yl)-1H-pyrazole (100.0 mg, 0.24 mmol, 1.00 equiv), N-(tert-butyl)-1-(6-iodopyridazin-3-yl)pyrrolidin-3-amine (100.3 mg, 0.29 mmol, 1.20 equiv), dioxane (3 mL), K₃PO₄ (128.0 mg, 0.60 mmol, 2.50 equiv), H₂O (0.6 mL), Sphos (9.9 mg, 0.024 mmol, 0.10 equiv), and Sphos-Pd-G3 (18.8 mg, 0.024 mmol, 0.10 equiv) was evacuated and flushed three times with nitrogen. The resulting solution was stirred for 16 h at 90° C., then quenched with water (30 mL), extracted with ethyl acetate (3×20 mL). The organic layers were combined, washed with saturated aqueous NaCl (1×50 mL), dried over anhydrous sodium sulfate, and filtered. The filtrate was concentrated under vacuum to give a residue. The residue was purified by reverse phase flash (Condition 9, Gradient 1) to afford N-(tert-butyl)-1-(6-(2-(methoxymethoxy)-4-(1-(tetrahydro-2H-pyran-2-yl)-1H-pyrazol-4-yl)phenyl)pyridazin-3-yl)pyrrolidin-3-amine (50.0 mg, 41%) as an oil. LCMS (ES, m/z): 507 [M+H]⁺.

### Synthesis of Compound 101

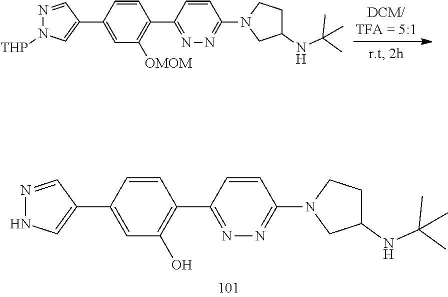

A mixture of N-(tert-butyl)-1-(6-(2-(methoxymethoxy)-4-(1-(tetrahydro-2H-pyran-2-yl)-1H-pyrazol-4-yl)phenyl)pyridazin-3-yl)pyrrolidin-3-amine (50.0 mg, 0.098 mmol, 1.00 equiv), DCM (1.0 mL), and TFA (0.2 mL) was stirred for 4 h at room temperature. The resulting mixture was concentrated under vacuum to give a residue. The residue was purified by Prep-HPLC (Condition 9, Gradient 1) to afford 2-(6-(3-(tert-butylamino)pyrrolidin-1-yl)pyridazin-3-yl)-5-(1H-pyrazol-4-yl)phenol as a solid (7.0 mg, 19%). LCMS (ES, m/z): 379 [M+H]^{+ 1}H-NMR (400 MHz, DMSO-d₆) δ 13.93 (s, 1H), 12.97 (s, 1H), 8.26 (s, 1H), 8.20 (d, J=9.8 Hz, 1H), 7.98 (s, 1H), 7.84 (d, J=8.3 Hz, 1H), 7.22-7.14 (m, 2H), 7.14 (d, J=9.7 Hz, 1H), 3.79 (t, J=8.6 Hz, 1H), 3.65 (s, 1H), 3.58-3.50 (m, 1H), 3.49-3.40 (m, 1H), 3.08 (dd, J=10.3, 7.1 Hz, 1H), 2.19 (s, 1H), 1.83-1.69 (m, 2H), 1.09 (s, 9H).

### Example 34: Synthesis of Compound 102

### Synthesis of Intermediate B79

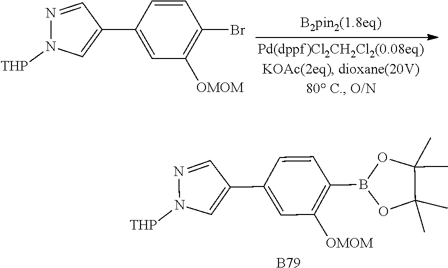

Into a 250-mL round-bottom flask, was placed 4-(4-bromo-3-(methoxymethoxy)phenyl)-1-(tetrahydro-2H-pyran-2-yl)-1H-pyrazole (3.25 g, 8.88 mmol, 1.00 equiv), bis(pinacolato)diboron (4.06 g, 15.98 mmol, 1.80 equiv), KOAc (1.74 g, 17.76 mmol, 2.00 equiv), Pd(dppf)Cl₂ CH₂Cl₂ (0.72 g, 0.888 mmol, 0.10 equiv) and dioxane (65 mL). The reaction mixture was evacuated and flushed three times with nitrogen. The resulting solution was stirred for 16 h at 80° C. The solids were filtered out. The reaction was then quenched by the addition of 100 mL of water. The resulting solution was extracted with 3×100 mL of ethyl acetate and the organic layers combined. The resulting mixture was washed with 1×300 ml of saturated aqueous NaCl. The mixture was dried over anhydrous sodium sulfate. The solids were filtered out. The resulting mixture was concentrated under vacuum. The residue was applied onto a silica gel column with ethyl acetate/petroleum ether to afford 1.7 g (46.2%) of 4-(3-(methoxymethoxy)-4-(4,4,5,5-tetramethyl-1,3,2-dioxaborolan-2-yl)phenyl)-1-(tetrahydro-2H-pyran-2-yl)-1H-pyrazole as an oil. LCMS (ES, m/z): 415 [M+H]⁺.

### Synthesis of Intermediate B80

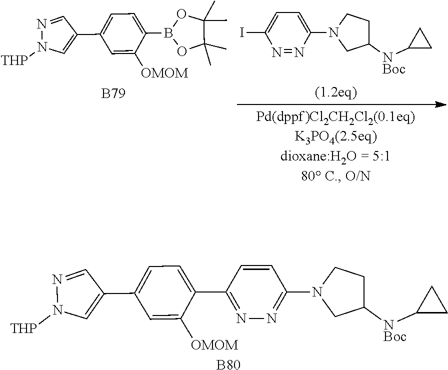

Into a 40-mL vial, was placed 4-(3-(methoxymethoxy)-4-(4,4,5,5-tetramethyl-1,3,2-dioxaborolan-2-yl) phenyl)-1-(tetrahydro-2H-pyran-2-yl)-1H-pyrazole (50.0 mg, 0.12 mmol, 1.00 equiv), tert-butyl cyclopsrop-yl(1-(6-iodopyridazin-3-yl)pyrrolidin-3-yl)carbamate (62.3 mg, 0.14 mmol, 1.20 equiv), dioxane (2 mL), K₃PO₄ (64.0 mg, 0.30 mmol, 2.50 equiv), H₂O (0.4 mL), Pd(dppf)Cl₂ CH₂Cl₂ (9.8 mg, 0.012 mmol, 0.10 equiv). The reaction mixture was evacuated and flushed three times with nitrogen. The resulting solution was stirred for 16 h at 80° C. The reaction was then quenched by the addition of 20 mL of water. The resulting solution was extracted with 3×20 mL of ethyl acetate and the organic layers combined. The resulting mixture was washed with 1×50 ml of saturated aqueous NaCl. The mixture was dried over anhydrous sodium sulfate. The solids were filtered out. The resulting mixture was concentrated under vacuum. The crude product was purified by reverse phase flash with the following conditions: (Water (10 MMOL/L NH₄HCO₃) and ACN) to afford 15.0 mg (21%) of tert-butyl cyclopropyl(1-(6-(2-hydroxy-4-(1-(tetrahydro-2H-pyran-2-yl)-1H-pyrazol-4-yl)phenyl)pyridazin-3-yl)pyrrolidin-3-yl)carbamate as a solid.

LCMS (ES, m/z): 591 [M+H]⁺.

### Synthesis of Compound 102

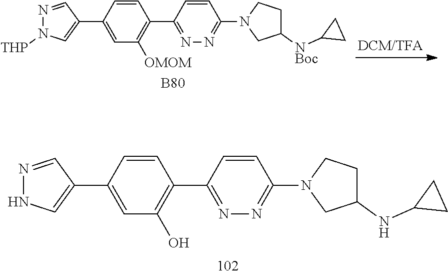

Into a 25-mL round-bottom flask, was tert-butyl cyclopropyl(1-(6-(2-hydroxy-4-(1-(tetrahydro-2H-pyran-2-yl)-1H-pyrazol-4-yl)phenyl)pyridazin-3-yl)pyrrolidin-3-yl)carbamate (15.0 mg, 0.025 mmol, 1.00 equiv), DCM (1.0 mL), TFA (0.2 mL). The resulting solution was stirred for 4 h at room temperature. The resulting mixture was concentrated under vacuum. The crude product was purified by Prep-HPLC (Condition 7, Gradient 1) to afford 4.2 mg (46%) of 2-(6-(3-(cyclopropylamino)pyrrolidin-1-yl)pyridazin-3-yl)-5-(1H-pyrazol-4-yl)phenol as a solid. LCMS (ES, m/z): 363 [M+H]⁺¹H-NMR (400 MHz, DMSO-d₆) δ 13.92 (s, 1H), 12.97 (s, 1H), 8.26 (s, 1H), 8.21 (d, J=9.9 Hz, 1H), 7.97 (s, 1H), 7.83 (d, J=8.3 Hz, 1H), 7.22-7.11 (m, 3H), 3.69 (dd, J=10.7, 5.9 Hz, 1H), 3.65-3.56 (m, 1H), 3.56-3.46 (m, 2H), 3.41-3.34 (m, 1H), 2.20-2.08 (m, 2H), 1.93 (dq, J=12.8, 6.4 Hz, 1H), 0.48-0.35 (m, 2H), 0.33-0.19 (m, 2H).

### Example 35: Synthesis of Compound 103

### Synthesis of Intermediate R81

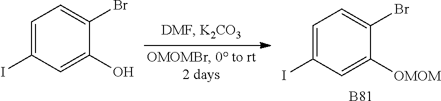

Into a 250 mL 3-necked round-bottom flask were added 2-bromo-5-iodophenol (10 g, 33.455 mmol, 1.00 equiv), DMF (50 mL) and K₂CO₃ (9.25 g, 66.910 mmol, 2.0 equiv) at room temperature. To the above mixture was added bromo(methoxy)methane (8.36 g, 66.910 mmol, 2.0 equiv) dropwise over 20 min at 0° C. The resulting mixture was stirred for additional 2 days at room temperature. The resulting mixture was diluted with water (100 mL). The resulting mixture was extracted with EtOAc (3×200 mL). The combined organic layers were washed with water (2×200 mL), dried over anhydrous Na₂SO₄. After filtration, the filtrate was concentrated under reduced pressure. The residue was purified by silica gel column chromatography, eluted with PE/EA (10:1) to afford 1-bromo-4-iodo-2-(methoxymethoxy)benzene (8 g, 69%) as an oil. LCMS (ES, m/z): 343 [M+H]⁺.

### Synthesis of Intermediate B82

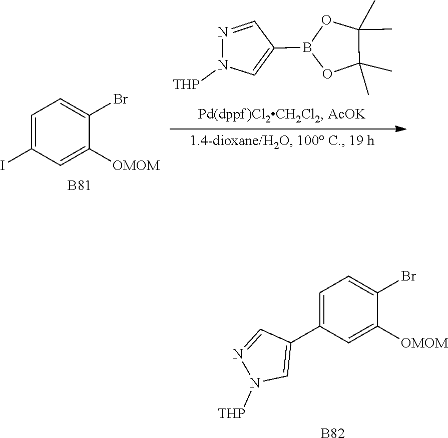

To a mixture of 1-bromo-4-iodo-2-(methoxymethoxy)benzene (8 g, 23.326 mmol, 1 equiv) and 1-(oxan-2-yl)-4-(4,4,5,5-tetramethyl-1,3,2-dioxaborolan-2-yl)pyrazole (6.81 g, 24.492 mmol, 1.05 equiv) in 1,4-dioxane (75 mL) and H₂O (15 mL) were added AcOK (6.87 g, 69.978 mmol, 3 equiv) and Pd(dppf)Cl₂·CH₂Cl₂ (1.90 g, 2.333 mmol, 0.1 equiv) at room temperature under nitrogen atmosphere. The mixture was stirred for 19 h at 100° C. under nitrogen atmosphere and concentrated under reduced pressure. The residue was purified by silica gel column chromatography, eluted with PE/EA (4:1) to afford 4-[4-bromo-3-(methoxymethoxy)phenyl]-1-(oxan-2-yl)pyrazole (7 g, 82%) as oil. LCMS (ES, m/z): 367 [M+H]⁺.

### Synthesis of Intermediate B83

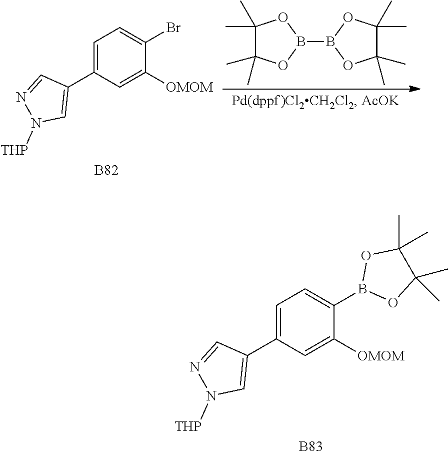

To a solution of 4-[4-bromo-3-(methoxymethoxy)phenyl]-1-(oxan-2-yl)pyrazole (5 g, 13.615 mmol, 1 equiv) and bis(pinacolato)diboron (4.15 g, 16.342 mmol, 1.20 equiv) in 1,4-dioxane (50 mL) were added AcOK (4.01 g, 40.845 mmol, 3.0 equiv) and Pd(dppf)Cl₂·CH₂Cl₂(1.11 g, 1.362 mmol, 0.1 equiv) at room temperature under nitrogen atmosphere. The mixture was stirred for 3 h at 100° C. under nitrogen atmosphere, and then concentrated under reduced pressure. The residue was purified by silica gel column chromatography, eluted with PE/EA (6:1) to afford 4-[3-(methoxymethoxy)-4-(4,4,5,5-tetramethyl-1,3,2-dioxaborolan-2-yl)phenyl]-1-(oxan-2-yl)pyrazole (3.9 g, 69%) as an oil. LCMS (ES, m/z): 415 [M+H]⁺.

### Synthesis of Intermediate B84

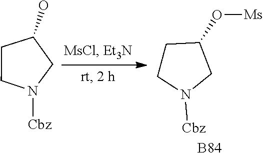 [SMILES: "B84" ; O=C(OCc1ccccc1)N1CC[C@H](O)C1 ; CS(=O)(=O)O[C@H]1CCN(C(=O)OCc2ccccc2)C1]

To a stirred solution of benzyl (3S)-3-hydroxypyrrolidine-1-carboxylate (10 g, 45.197 mmol, 1 equiv) and Et₃N (9.15 g, 90.394 mmol, 2.0 equiv) in DCM (100 mL) was added MsCl (6.21 g, 54.236 mmol, 1.2 equiv) dropwise at room temperature. After stirred for 2 hr at room temperature, the resulting mixture was washed with water. The organic phase was concentrated under reduced pressure to afford benzyl (S)-3-((methylsulfonyl)oxy)pyrrolidine-1-carboxylate (12.8 g, 95%) as an oil. The crude product was used in the next step directly without further purification. LCMS (ES, m/z): 300 [M+H]⁺.

### Synthesis of Intermediate B85

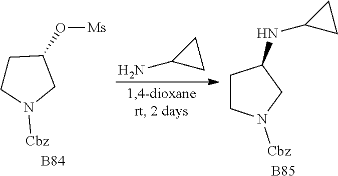

To a stirred solution of benzyl (3S)-3-(methanesulfonyloxy)pyrrolidine-1-carboxylate (13 g, 43.429 mmol, 1 equiv) in 1,4-dioxane (50 mL) was added aminocyclopropane (24.80 g, 434.290 mmol, 10 equiv) at room temperature. The resulting mixture was stirred for additional 2 days at room temperature. The resulting mixture was concentrated under reduced pressure. The residue was purified by silica gel column chromatography, eluted with PE/EA (10:1) to afford benzyl (3R)-3-(cyclopropylamino)pyrrolidine-1-carboxylate (7 g, 62%) as an oil. LCMS (ES, m/z): 261 [M+H]⁺.

### Synthesis of Intermediate B86

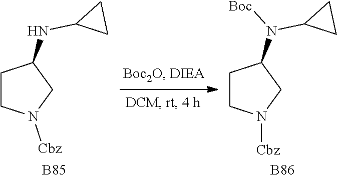 [SMILES: "B85" ; O=C(OCc1ccccc1)N1CC[C@@H](NC2CC2)C1 ; "B86" ; CC(C)(C)OC(=O)N(C1CC1)[C@@H]1CCN(C(=O)OCc2ccccc2)C1]

To a stirred solution of benzyl (3R)-3-(cyclopropylamino)pyrrolidine-1-carboxylate (6.6 g, 25.352 mmol, 1 equiv) and Boc₂O (8.30 g, 38.028 mmol, 1.5 equiv) in DCM (66 mL) was added DIEA (6.55 g, 50.704 mmol, 2.0 equiv) at room temperature. The resulting mixture was stirred for 4 h at room temperature. The resulting mixture was concentrated under reduced pressure. The residue was purified by silica gel column chromatography, eluted with PE/EA (8:1) to afford benzyl (3R)-3-[(tert-butoxycarbonyl)(cyclopropyl)amino]pyrrolidine-1-carboxylate (6.9 g, 76%) as an oil. LCMS (ES, m/z): 361 [M+H]⁺.

### Synthesis of Intermediate B87

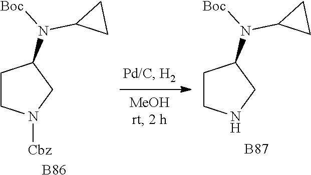 [SMILES: "B86" ; CC(C)(C)OC(=O)N(C1CC1)[C@@H]1CCN(C(=O)OCc2ccccc2)C1 ; "B87" ; CC(C)(C)OC(=O)N(C1CC1)[C@@H]1CCNC1]

To a solution of benzyl (3R)-3-[(tert-butoxycarbonyl)(cyclopropyl)amino]pyrrolidine-1-carboxylate (6.7 g, 18.588 mmol, 1 equiv) in 120 mL MeOH was added Pd/C (10%, 0.4 g) under nitrogen atmosphere in a 250 mL round-bottom flask. The mixture was hydrogenated at room temperature for 2 h under hydrogen atmosphere using a hydrogen balloon, filtered through a Celite pad and concentrated under reduced pressure to afford tert-butyl N-cyclopropyl-N-[(3R)-pyrrolidin-3-yl]carbamate (4 g, 95%) as an oil. The crude product was used in the next step directly without further purification. LCMS (ES, m/z): 227 [M+H]⁺.

### Synthesis of Intermediate B88

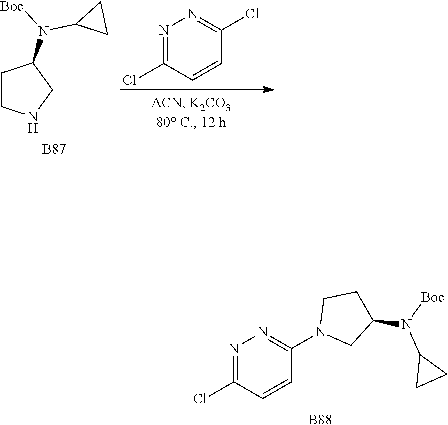 [SMILES: "B87" ; CC(C)(C)OC(=O)N(C1CC1)[C@@H]1CCNC1 ; "B88" ; Clc1ccc(Cl)nn1 ; CC(C)(C)OC(=O)N(C1CC1)[C@@H]1CCN(c2ccc(Cl)nn2)C1]

To a stirred mixture of tert-butyl N-cyclopropyl-N-[(3R)-pyrrolidin-3-yl]carbamate (0.5 g, 2.209 mmol, 1 equiv) and pyridazine, 3,6-dichloro- (0.39 g, 2.651 mmol, 1.2 equiv) in ACN (7.5 mL) was added K₂CO₃ (0.92 g, 6.627 mmol, 3 equiv) at room temperature.The resulting mixture was stirred for 12 h at 80° C. and filtered. The filtrated was concentrated under reduced pressure.

The residue was purified by silica gel column chromatography, eluted with PE/EA (4:1) to afford tert-butyl N-[(3R)-1-(6-chloropyridazin-3-yl)pyrrolidin-3-yl]-N-cyclopropylcarbamate (0.56 g, 75%) as a solid. LCMS (ES, m/z): 339 [M+H]⁺.

### Synthesis of Intermediate B89

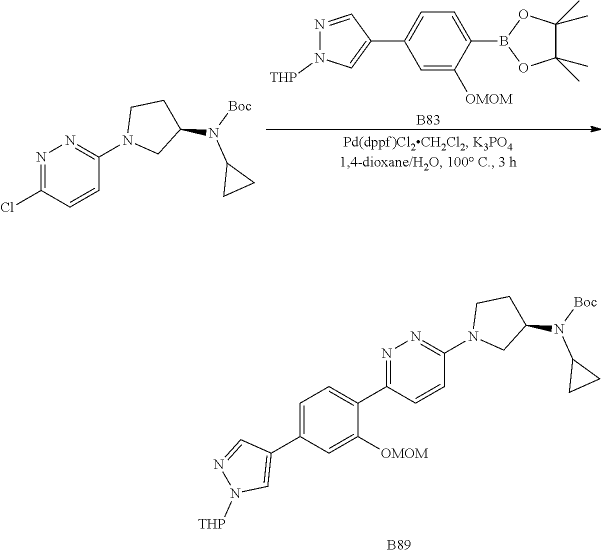

To a solution of tert-butyl (R)-(1-(6-chloropyridazin-3-yl)pyrrolidin-3-yl)(cyclopropyl)carbamate (480 mg, 1.417 mmol, 1 equiv) and 4-(3-(methoxymethoxy)-4-(4,4,5,5-tetramethyl-1,3,2-dioxaborolan-2-yl)phenyl)-1-(tetrahydro-2H-pyran-2-yl)-1H-pyrazole (762.98 mg, 1.842 mmol, 1.3 equiv) in 1,4-dioxane (5 mL) and H₂O (1 mL) were added K₃PO₄ (902.09 mg, 4.251 mmol, 3 equiv) and Pd(dppf)Cl₂·CH₂Cl₂ (115.40 mg, 0.142 mmol, 0.1 equiv) at room temperature under nitrogen atmosphere. After stirred for 3 h at 100° C. under nitrogen atmosphere, the resulting mixture was concentrated under reduced pressure. The residue was purified by silica gel column chromatography, eluted with CH₂Cl₂/MeOH (50:1) to afford tert-butyl cyclopropyl((3R)-1-(6-(2-(methoxymethoxy)-4-(1-(tetrahydro-2H-pyran-2-yl)-1H-pyrazol-4-yl)phenyl)pyridazin-3-yl)pyrrolidin-3-yl)carbamate (270 mg, 32%) as a solid. LCMS (ES, m/z): 591 [M+H]⁺.

### Synthesis of Compound 103

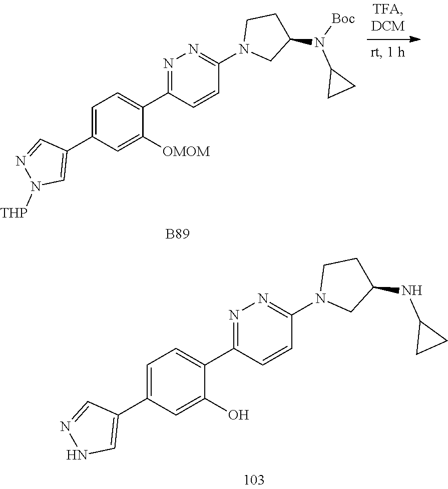

To a stirred solution of tert-butyl cyclopropyl((3R)-1-(6-(2-(methoxymethoxy)-4-(1-(tetrahydro-2H-pyran-2-yl)-1H-pyrazol-4-yl)phenyl)pyridazin-3-yl)pyrrolidin-3-yl)carbamate (270 mg, 0.457 mmol, 1 equiv) in DCM (2.5 mL) was added TFA (2.5 mL) at room temperature. The resulting mixture was stirred for 1 hr at room temperature and concentrated under reduced pressure. The crude product was purified by chiral SFC with the following conditions (Column: Lux 5 um Cellulose-4, 3*25 cm, 5 m; Mobile Phase A: CO₂, Mobile Phase B: MeOH:ACN=1:1(0.1% 2M NH₃-MeOH); Flow rate: 120 mL/min; Gradient: isocratic 45% B; Column Temperature (° C.): 35; Back Pressure(bar): 100; Wave Length: 220 nm; RT1(min): 17.5; RT2(min): 19.5; Sample Solvent: MeOH:ACN=1: 1; Injection Volume: 0.5 mL; Number Of Runs: 35) to afford (R)-2-(6-(3-(cyclopropylamino)pyrrolidin-1-yl)pyridazin-3-yl)-5-(1H-pyrazol-4-yl)phenol (102 mg, 62%) as solid. LCMS (ES, m/z): 363[M+H]^{+ 1}H NMR (400 MHz, DMSO-d₆) δ 13.91 (s, 1H), 12.97 (s, 1H), 8.30-8.17 (m, 2H), 7.97 (s, 1H), 7.83 (d, J=8.2 Hz, 1H), 7.23-7.11 (m, 3H), 3.73-3.47 (m, 4H), 3.37 (dd, J=10.4, 4.7 Hz, 1H), 3.39-3.35 (m, 1H), 2.20-2.08 (m, 2H), 1.93 (dq, J=12.8, 6.5 Hz, 1H), 0.47-0.36 (m, 2H), 0.31-0.20 (m, 2H).

### Example 36: Synthesis of Compound 104

### Synthesis of Intermediate B90

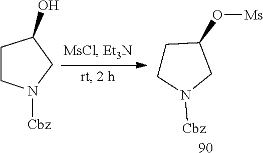

To a stirred solution of benzyl (3R)-3-hydroxypyrrolidine-1-carboxylate (10 g, 45.197 mmol, 1 equiv) and Et₃N (9.15 g, 90.394 mmol, 2 equiv) in DCM (100 mL) was added MsCl (6.21 g, 54.236 mmol, 1.2 equiv) dropwise at room temperature. After stirring for 2 h at room temperature, the resulting mixture was concentrated under reduced pressure to afford benzyl (3R)-3-(methanesulfonyloxy)pyrrolidine-1-carboxylate (12.5 g, 92.39%) as an oil. The crude product was used in the next step directly without further purification. LCMS (ES, m/z): 300 [M+H]⁺.

### Synthesis of Intermediate B91

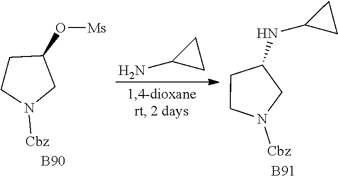

To a stirred solution of benzyl (3R)-3-(methanesulfonyloxy)pyrrolidine-1-carboxylate (12 g, 40.088 mmol, 1 equiv) in 1,4-dioxane (50 mL) was added aminocyclopropane (22.89 g, 400 mmol, 10 equiv). The resulting mixture was stirred for 2 days at room temperature. The resulting mixture was concentrated under reduced pressure. The residue was purified by silica gel column chromatography, eluted with PE/EA (10:1) to afford benzyl (3S)-3-(cyclopropylamino)pyrrolidine-1-carboxylate (6 g, 57%) as am oil. LCMS (ES, m/z): 261 [M+H]⁺.

### Synthesis of Intermediate B92

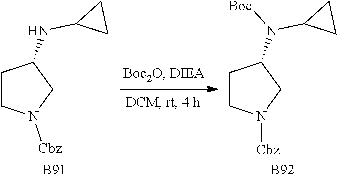 [SMILES: "B91" ; O=C(OCc1ccccc1)N1CC[C@H](NC2CC2)C1 ; "B92" ; CC(C)(C)OC(=O)N(C1CC1)[C@H]1CCN(C(=O)OCc2ccccc2)C1]

To a stirred solution of benzyl benzyl (3S)-3-(cyclopropylamino)pyrrolidine-1-carboxylate (6 g, 23.047 mmol, 1 equiv) and Boc₂O (7.54 g, 34.571 mmol, 1.5 equiv) in DCM (60 mL) was added DIEA (5.96 g, 46.094 mmol, 2 equiv) dropwise at room temperature. The resulting mixture was stirred for 4 hr at room temperature. The resulting mixture was concentrated under reduced pressure. The residue was purified by silica gel column chromatography, eluted with PE/EA(8:1) to afford benzyl (3S)-3-[(tert-butoxycarbonyl)(cyclopropyl)amino]pyrrolidine-1-carboxylate (5.2 g, 63%) as an oil. LCMS (ES, m/z): 361 [M+H]⁺.

### Synthesis of Intermediate B93

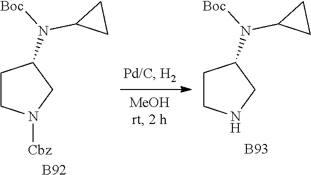 [SMILES: "B92" ; CC(C)(C)OC(=O)N(C1CC1)[C@H]1CCN(C(=O)OCc2ccccc2)C1 ; "B93" ; CC(C)(C)OC(=O)N(C1CC1)[C@H]1CCNC1]

To a solution of benzyl (3S)-3-[(tert-butoxycarbonyl)(cyclopropyl)amino]pyrrolidine-1-carboxylate (5.2 g, 14.426 mmol, 1 equiv) in MEOH (110 mL) was added Pd/C (10%, 0.4 g) under nitrogen atmosphere in a 250 mL round-bottom flask. The mixture was hydrogenated at room temperature for 2 h under hydrogen atmosphere using a hydrogen balloon. The mixture was filtered through a Celite pad and concentrated under reduced pressure to afford tert-butyl N-cyclopropyl-N-[(3S)-pyrrolidin-3-yl]carbamate (3 g, 92%) as an oil. The crude product was used in the next step directly without further purification. LCMS (ES, m/z): 227 [M+H]⁺.

### Synthesis of Intermediate B94

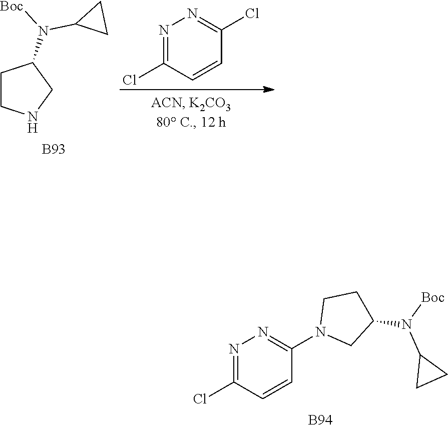 [SMILES: "B93" ; CC(C)(C)OC(=O)N(C1CC1)[C@H]1CCNC1 ; "B94" ; Clc1ccc(Cl)nn1 ; CC(C)(C)OC(=O)N(C1CC1)[C@H]1CCN(c2ccc(Cl)nn2)C1]

To a stirred mixture of tert-butyl N-cyclopropyl-N-[(3S)-pyrrolidin-3-yl]carbamate (500 mg, 2.209 mmol, 1 equiv) and 3,6-dichloropyridazine (394.94 mg, 2.651 mmol, 1.2 equiv) in ACN (5 mL) was added K₂CO₃ (916.00 mg, 6.627 mmol, 3 equiv) in portions at room temperature. The resulting mixture was stirred for 12 h at 80° C. The resulting mixture was filtered. The filtrate was concentrated under reduced pressure. The residue was purified by silica gel column chromatography, eluted with PE/EA(4:1) to afford tert-butyl N-[(3S)-1-(6-chloropyridazin-3-yl)pyrrolidin-3-yl]-N-cyclopropylcarbamate (500 mg, 67%) as a solid.

LCMS (ES, m/z): 339 [M+H]⁺.

### Synthesis of Intermediate B95

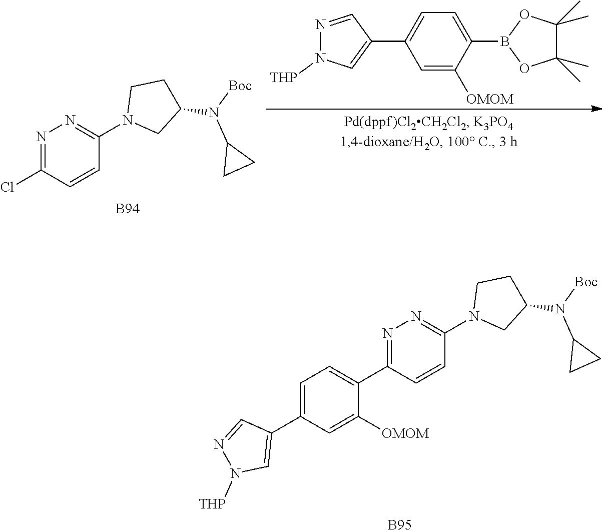

To a solution of tert-butyl N-[(3S)-1-(6-chloropyridazin-3-yl)pyrrolidin-3-yl]-N-cyclopropylcarbamate (480 mg, 1.417 mmol, 1 equiv) and 4-[3-(methoxymethoxy)-4-(4,4,5,5-tetramethyl-1,3,2-dioxaborolan-2-yl)phenyl]-1-(oxan-2-yl)pyrazole (762.98 mg, 1.842 mmol, 1.3 equiv) in 1,4-dioxane (5 mL) and H2O (1 mL) was added K₃PO₄ (902.09 mg, 4.251 mmol, 3 equiv) and Pd(dppf)Cl₂—CH₂Cl₂ (115.40 mg, 0.142 mmol, 0.1 equiv) at room temperature under nitrogen atmosphere. The resulting mixture was stirred for 3 hr at 80° C. under nitrogen atmosphere. The resulting mixture was concentrated under reduced pressure. The residue was purified by silica gel column chromatography, eluted with CH₂Cl₂/MeOH(50:1) to afford tert-butyl N-cyclopropyl-N-[(3S)-1-{6-[2-(methoxymethoxy)-4-[1-(oxan-2-yl)pyrazol-4-yl]phenyl]pyridazin-3-yl}pyrrolidin-3-yl]carbamate (300 mg, 36%) as a solid. LCMS (ES, m/z): 591 [M+H]⁺.

### Synthesis of Compound 104

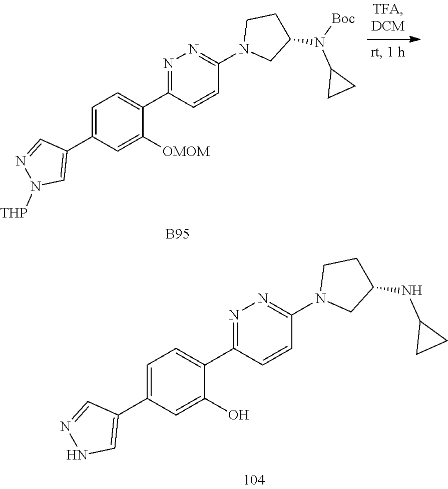

To a stirred solution of tert-butyl N-cyclopropyl-N-[(3S)-1-{6-[2-(methoxymethoxy)-4-[1-(oxan-2-yl)pyrazol-4-yl]phenyl]pyridazin-3-yl}pyrrolidin-3-yl]carbamate (300 mg, 0.508 mmol, 1 equiv) in DCM (3 mL) was added TFA (3 mL) at room temperature. The resulting mixture was stirred for 1 h at room temperature and concentrated under reduced pressure. The crude product was purified by Prep-HPLC (Condition 4, Gradient 3). The crude product was further purified by chiral Prep-HPLC with the following conditions (Column: Lux 5 um Cellulose-2, 3*25 cm, 5 m; Mobile Phase A: CO₂, Mobile Phase B: MeOH:ACN=1: 1(0.1% 2M NH₃-MeOH); Flow rate: 90 mL/min; Gradient: isocratic 60% B; Column Temperature(° C.): 35; Back Pressure(bar): 100; Wave Length: 220 nm; RT1(min): 8.15; RT2(min): 9.83; Sample Solvent: MeOH-Preparative; Injection Volume: 3 mL; Number Of Runs: 10) to afford 2-{6-[(3S)-3-(cyclopropylamino)pyrrolidin-1-yl]pyridazin-3-yl}-5-(1H-pyrazol-4-yl)phenol; ethane (175 mg, 88%) as solid LCMS (ES, m/z): 363[M+H]^{+ 1}H NMR (400 MHz, DMSO-d6) δ 13.92 (s, 1H), 12.97 (s, 1H), 8.30-8.17 (m, 2H), 7.97 (s, 1H), 7.83 (d, J=8.2 Hz, 1H), 7.25-7.14 (m, 3H), 3.69 (dd, J=10.6, 5.9 Hz, 1H), 3.64-3.56 (m, 1H), 3.52 (dd, J=10.3, 5.7 Hz, 2H), 3.39-3.30 (m, 2H), 2.12 (td, J=6.8, 3.9 Hz, 2H), 1.93 (dq, J=12.7, 6.6 Hz, 1H), 0.42 (dd, J=6.6, 1.6 Hz, 2H), 0.31-0.20 (m, 2H).

### Example 37: Synthesis of Compound 106

### Synthesis of Intermediate B96

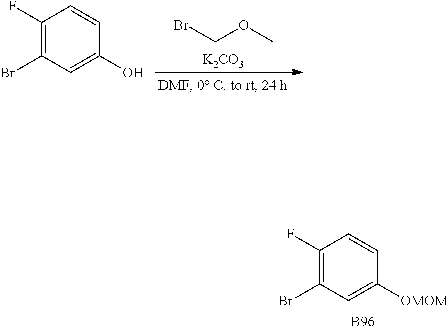

Into a 100 mL 3-necked round-bottom flask was added 3-bromo-4-fluorophenol (5 g, 26.178 mmol, 1 equiv), DMF (50 mL) and K₂CO₃ (7.29 g, 52.356 mmol, 2.0 equiv) at room temperature. To the above mixture was added bromo(methoxy)methane (3.43 g, 27.487 mmol, 1.05 equiv) dropwise over 20 min at 0° C. The resulting mixture was stirred for additional 1 day at room temperature. The resulting mixture was diluted with water (50 mL) and extracted with EtOAc (3×100 mL). The combined organic layers were washed with water (2×100 mL), dried over anhydrous Na₂SO₄. After filtration, the filtrate was concentrated under reduced pressure. The residue was purified by silica gel column chromatography, eluted with PE/EA (10:1) to afford 2-bromo-1-fluoro-4-(methoxymethoxy)benzene (3 g, 49%) as an oil. LCMS (ES, m/z): 235 [M+H]⁺.

### Synthesis of Intermediate B97

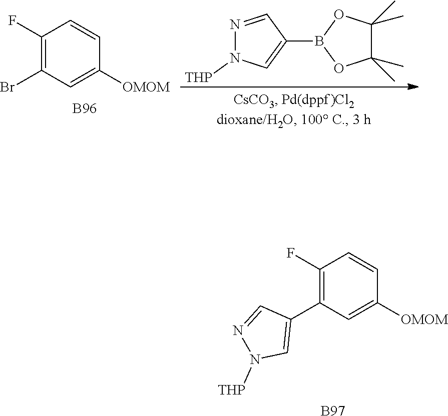

To a mixture of 2-bromo-1-fluoro-4-(methoxymethoxy)benzene (2 g, 8.509 mmol, 1 equiv) and 1-(oxan-2-yl)-4-(4,4,5,5-tetramethyl-1,3,2-dioxaborolan-2-yl)pyrazole (2.49 g, 8.934 mmol, 1.05 equiv) in 1,4-dioxane (100 mL) and H₂O (20 mL) was added Cs₂CO₃ (8.32 g, 25.527 mmol, 3 equiv) and Pd(dppf)Cl₂—CH₂Cl₂(0.69 g, 0.851 mmol, 0.1 equiv) at room temperature under nitrogen atmosphere. The resulting mixture was stirred for 3 h at 100° C. under nitrogen atmosphere, the resulting mixture was concentrated under reduced pressure. The residue was purified by silica gel column chromatography, eluted with PE/EA (4:1) to afford 4-[2-fluoro-5-(methoxymethoxy)phenyl]-1-(oxan-2-yl)pyrazole (2.3 g, 88%) as an oil. LCMS (ES, m/z): 307 [M+H]⁺.

### Synthesis of Intermediate B98

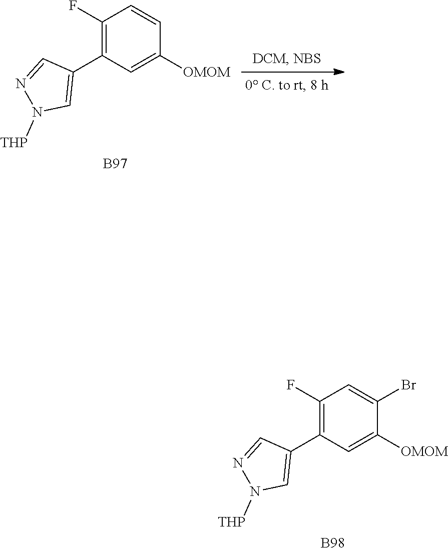

To a stirred solution of 4-[2-fluoro-5-(methoxymethoxy)phenyl]-1-(oxan-2-yl)pyrazole (600 mg, 1.959 mmol, 1 equiv) in DCM (6 mL) was added NBS (331.17 mg, 1.861 mmol, 0.95 equiv) in portions at 0° C. The resulting mixture was stirred for 8 hr at room temperature. The resulting mixture was concentrated under reduced pressure. The residue was purified by silica gel column chromatography, eluted with PE/EA (4:1) to afford 4-[4-bromo-2-fluoro-5-(methoxymethoxy)phenyl]-1-(oxan-2-yl)pyrazole (600 mg, 80%) as an oil. LCMS (ES, m/z): 385 [M+H]⁺.

### Synthesis of Intermediate B99

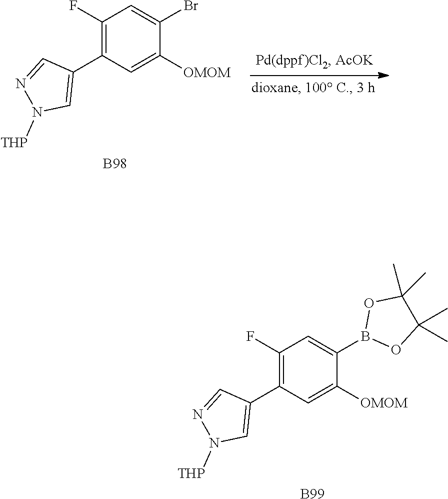

To a stirred solution of 4-[4-bromo-2-fluoro-5-(methoxymethoxy)phenyl]-1-(oxan-2-yl)pyrazole (300 mg, 0.779 mmol, 1 equiv) and bis(pinacolato)diboron (296.63 mg, 1.169 mmol, 1.5 equiv) in dioxane (3 mL) was added AcOK (229.28 mg, 2.337 mmol, 3 equiv) and Pd(dppf)Cl₂ (56.98 mg, 0.078 mmol, 0.1 equiv) at room temperature under nitrogen atmosphere. The resulting mixture was stirred for 3 hr at 100° C. under nitrogen atmosphere. The resulting mixture was concentrated under reduced pressure. The residue was purified by silica gel column chromatography, eluted with PE/EA (5:1) to afford 4-[2-fluoro-5-(methoxymethoxy)-4-(4,4,5,5-tetramethyl-1,3,2-dioxaborolan-2-yl)phenyl]-1-(oxan-2-yl)pyrazole (270 mg, 80%) as an oil.

LCMS (ES, m/z): 433 [M+H]⁺

### Synthesis of Intermediate B100

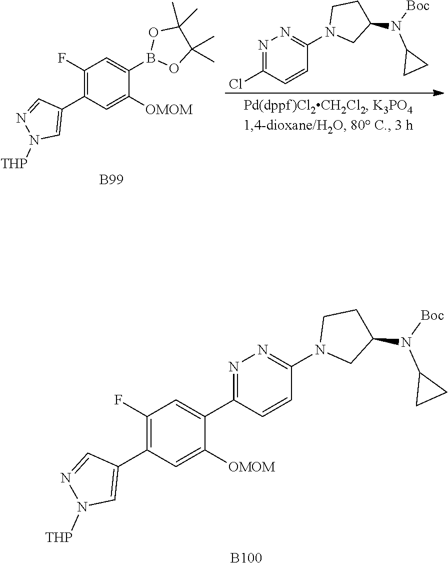

To a solution of 4-[2-fluoro-5-(methoxymethoxy)-4-(4,4,5,5-tetramethyl-1,3,2-dioxaborolan-2-yl)phenyl]-1-(oxan-2-yl)pyrazole (80 mg, 0.185 mmol, 1.3 equiv) and tert-butyl N-[(3R)-1-(6-chloropyridazin-3-yl)pyrrolidin-3-yl]-N-cyclopropylcarbamate (48.23 mg, 0.142 mmol, 1 equiv) in 1,4-dioxane (4 mL) and H₂O (0.8 mL) was added K₃PO₄ (90.65 mg, 0.427 mmol, 3 equiv) and Pd(dppf)Cl₂—CH₂Cl₂ (11.60 mg, 0.014 mmol, 0.1 equiv) at room temperature under nitrogen atmosphere. The resulting mixture was stirred for 3 h at 80° C. under nitrogen atmosphere, the resulting mixture was concentrated under reduced pressure. The residue was purified by silica gel column chromatography, eluted with CH₂Cl₂/MeOH (50:1) to afford tert-butyl N-cyclopropyl-N-[(3R)-1-{6-[5-fluoro-2-(methoxymethoxy)-4-[1-(oxan-2-yl)pyrazol-4-yl]phenyl]pyridazin-3-yl}pyrrolidin-3-yl]carbamate (60 mg, 69%) as a solid. LCMS (ES, m/z): 609 [M+H]⁺.

### Synthesis of Compound 106

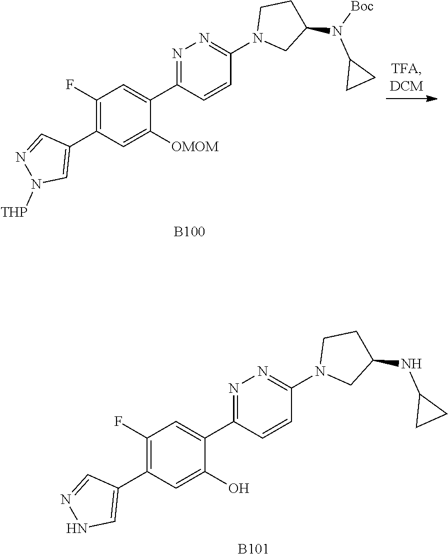

To a stirred solution of tert-butyl N-cyclopropyl-N-[(3R)-1-{6-[5-fluoro-2-(methoxymethoxy)-4-[1-(oxan-2-yl)pyrazol-4-yl]phenyl]pyridazin-3-yl}pyrrolidin-3-yl]carbamate (60 mg, 0.099 mmol, 1 equiv) in DCM (0.06 mL, 0.948 mmol, 9.58 equiv) was added TFA (0.06 mL, 0.812 mmol, 8.20 equiv) at room temperature. The resulting mixture was stirred for 1 hr at room temperature under. The resulting mixture was concentrated under reduced pressure. The crude product was first purified by reverse phase flash chromatography (Condition 4, Gradient 2). The product was further purified by chiral SFC with the following conditions (Column: Lux 5 um Cellulose-2, 3*25 cm, 5 m; Mobile Phase A: CO₂, Mobile Phase B: MeOH: ACN=1: 1(0.1% 2M NH₃-MeOH); Flow rate: 80 mL/min; Gradient: isocratic 50% B; Column Temperature(° C.): 35; Back Pressure(bar): 100; Wave Length: 220 nm; RT1(min): 16; RT2(min): 19; Sample Solvent: MeOH-HPLC; Injection Volume: 1 mL; Number Of Runs: 10) to afford 2-{6-[(3R)-3-(cyclopropylamino)pyrrolidin-1-yl]pyridazin-3-yl}-4-fluoro-5-(1H-pyrazol-4-yl)phenol (15 mg, 40%) as a solid LCMS (ES, m/z):509[M+H]^{+ 1}H NMR (400 MHz, DMSO-d6) δ 13.60 (s, 1H), 13.13 (s, 1H), 8.25 (t, J=8.7 Hz, 2H), 7.99 (s, 1H), 7.83 (d, J=12.7 Hz, 1H), 7.30 (d, J=7.0 Hz, 1H), 7.19 (d, J=9.8 Hz, 1H), 3.80-3.60 (m, 2H), 3.59-3.44 (m, 2H), 2.41-2.16 (m, 2H), 2.04 (s, 1H), 0.54 (d, J=6.6 Hz, 2H), 0.42 (s, 2H).

### Example 38: Synthesis of Compound 105

### Synthesis of Intermediate B102

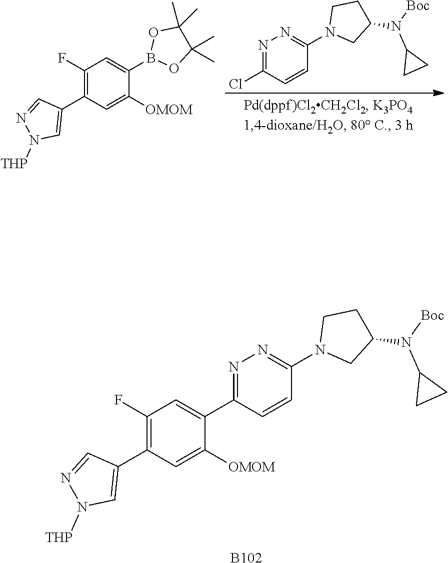

To a solution of 4-[2-fluoro-5-(methoxymethoxy)-4-(4,4,5,5-tetramethyl-1,3,2-dioxaborolan-2-yl)phenyl]-1-(oxan-2-yl)pyrazole (100 mg, 0.231 mmol, 1.3 equiv) and tert-butyl (S)-(1-(6-chloropyridazin-3-yl)pyrrolidin-3-yl)(cyclopropyl)carbamate (60.29 mg, 0.178 mmol, 1 equiv) in 1,4-dioxane (5 mL) and H₂O (1 mL) was added K₃PO₄ (113.31 mg, 0.533 mmol, 3 equiv) and Pd(dppf)Cl₂—CH₂Cl₂ (14.50 mg, 0.018 mmol, 0.1 equiv) at room temperature under nitrogen atmosphere. The resulting mixture was stirred for 3 hr at 80° C. under nitrogen atmosphere, the resulting mixture was concentrated under reduced pressure. The residue was purified by silica gel column chromatography, eluted with CH₂Cl₂/MeOH (50:1) to afford tert-butyl N-cyclopropyl-N-[(3S)-1-{6-[5-fluoro-2-(methoxymethoxy)-4-[1-(oxan-2-yl)pyrazol-4-yl]phenyl]pyridazin-3-yl}pyrrolidin-3-yl]carbamate (60 mg, 55%) as a solid. LCMS (ES, m/z): 609 [M+H]⁺.

### Synthesis of Compound 105

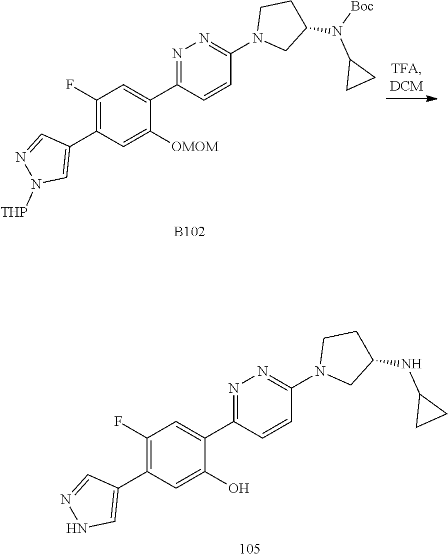

To a stirred solution of tert-butyl N-cyclopropyl-N-[(3S)-1-{6-[5-fluoro-2-(methoxymethoxy)-4-[1-(oxan-2-yl)pyrazol-4-yl]phenyl]pyridazin-3-yl}pyrrolidin-3-yl]carbamate (60 mg, 0.099 mmol, 1 equiv) and DCM (0.6 mL) was added TFA (0.6 mL) at room temperature. The resulting mixture was stirred for 1 h at room temperature. The resulting mixture was concentrated under reduced pressure. The crude product was purified by reverse phase flash chromatography (Condition 4, Gradient 2). The product was purified by chiral SFC with the following conditions (Column: Lux 5 um Cellulose-2, 3*25 cm, 5 m; Mobile Phase A: C02, Mobile Phase B: MeOH:ACN=3: 1(0.1% 2 mM NH₃-MeOH); Flow rate: 80 mL/min; Gradient: isocratic 50% B; Column Temperature(° C.): 35; Back Pressure(bar): 100; Wave Length: 220 nm; RT1(min): 16; RT2(min): 19; Sample Solvent: MeOH-HPLC; Injection Volume: 2 mL; Number Of Runs: 5) to afford 2-{6-[(3R)-3-(cyclopropylamino)pyrrolidin-1-yl]pyridazin-3-yl}-4-fluoro-5-(1H-pyrazol-4-yl)phenol (15 mg, 40%) as a solid. LCMS (ES, m/z): 381[M+H]^{+ 1}H NMR (400 MHz, DMSO-d6) δ 8.96 (d, J=25.0 Hz, 2H), 8.32 (d, J=9.8 Hz, 1H), 8.11 (s, 2H), 7.86 (d, J=12.7 Hz, 1H), 7.30 (dd, J=8.3, 2.8 Hz, 2H), 4.14 (s, 1H), 3.92 (dd, J=12.1, 6.3 Hz, 1H), 3.82 (dd, J=12.1, 4.2 Hz, 1H), 3.71 (t, J=7.8 Hz, 1H), 3.64 (dd, J=8.6, 6.0 Hz, 1H), 2.88 (s, 1H), 2.49-2.44 (m, 2H), 2.46-2.41 (m, 1H), 2.29 (dd, J=12.8, 6.8 Hz, 1H), 0.86 (d, J=5.7 Hz, 4H).

### Example 39: Synthesis of Compound 107

### Synthesis of Intermediate B103

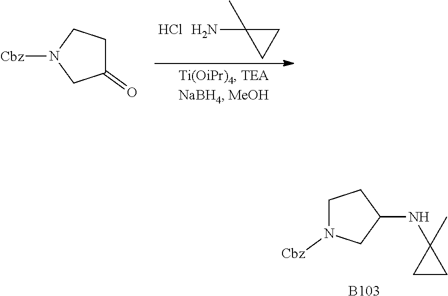

To a stirred mixture of 1-methylcyclopropan-1-amine hydrochloride (4.32 g, 40.139 mmol, 2.2 equiv) and TEA (4.06 g, 40.139 mmol, 2.2 equiv) was added benzyl 3-oxopyrrolidine-1-carboxylate (4 g, 18.245 mmol, 1 equiv) and Ti(Oi-Pr)₄ (4 mL) at room temperature under nitrogen atmosphere. The resulting mixture was stirred for 2 h at room temperature under nitrogen atmosphere. The reaction mixture was added MeOH (40 mL) and then NaBH₄ (1.24 g, 32.841 mmol, 1.8 equiv) was added in portions at 0° C. The resulting mixture was stirred for 2 h at room temperature under nitrogen atmosphere. The reaction was quenched with water/ice at room temperature. The resulting mixture was extracted with EtOAc (2×100 mL). The combined organic layers were washed with brine (1×100 mL), dried over anhydrous Na₂SO₄. After filtration, the filtrate was concentrated under reduced pressure. The residue was purified by silica gel column chromatography, eluted with PE/THF (2:1) to afford benzyl 3-[(1-methylcyclopropyl)amino]pyrrolidine-1-carboxylate (1.2 g, 24%) as an oil. LCMS (ES, m/z): 275 [M+H]⁺.

### Synthesis of Intermediate B104

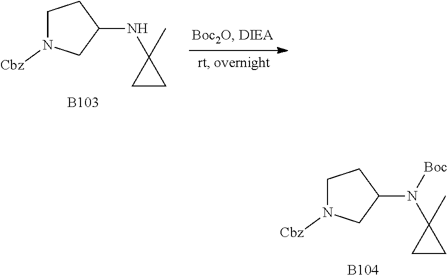

To a stirred mixture of benzyl 3-[(1-methylcyclopropyl)amino]pyrrolidine-1-carboxylate (1.3 g, 4.738 mmol, 1 equiv) and Boc₂O (1.55 g, 7.107 mmol, 1.5 equiv) in DCM (13 mL) was added DIEA (1.22 g, 9.476 mmol, 2.0 equiv) dropwise at room temperature. The resulting mixture was stirred overnight at room temperature. The resulting mixture was concentrated under reduced pressure. The residue was purified by silica gel column chromatography, eluted with PE/THE (5:1) to afford benzyl 3-[(tert-butoxycarbonyl)(1-methylcyclopropyl)amino]pyrrolidine-1-carboxylate (1.2 g, 68%) as an oil. LCMS (ES, m/z): 375 [M+H]⁺.

### Synthesis of Intermediate B105

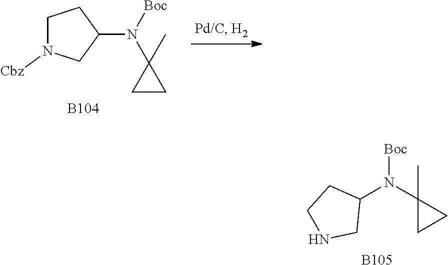

To a solution of benzyl (3R)-3-[(tert-butoxycarbonyl)(cyclopropyl)amino]pyrrolidine-1-carboxylate (1.2 g, 3.329 mmol, 1 equiv) in 120 mL MeOH was added Pd/C (10%, 0.4 g) under nitrogen atmosphere in a 250 mL round-bottom flask. The mixture was hydrogenated at room temperature for 2 hr under hydrogen atmosphere using a hydrogen balloon. The mixture was filtered through a Celite pad and the filtrate was concentrated under reduced pressure to afford tert-butyl N-cyclopropyl-N-[(3R)-pyrrolidin-3-yl]carbamate (0.6 g, 80%) as an oil. The crude product was used in the next step directly without further purification. LCMS (ES, m/z): 241 [M+H]⁺.

### Synthesis of Intermediate B106

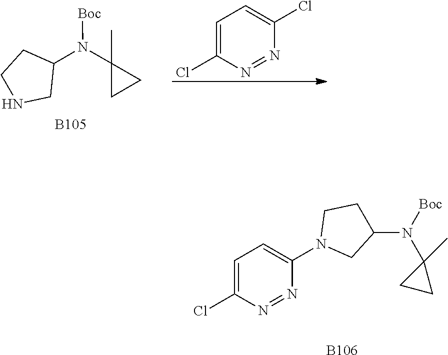

To a stirred mixture of tert-butyl N-(1-methylcyclopropyl)-N-(pyrrolidin-3-yl)carbamate (150 mg, 0.624 mmol, 1 equiv) and 3,6-dichloro pyridazine (111.57 mg, 0.749 mmol, 1.2 equiv) in ACN (1.5 mL) was added K₂CO₃ (258.76 mg, 1.872 mmol, 3.00 equiv) at room temperature. The resulting mixture was stirred for 12 hr at 80° C. The resulting mixture was filtered and the filtrated was concentrated under reduced pressure. The residue was purified by silica gel column chromatography, eluted with PE/EA (4:1) to afford tert-butyl N-[1-(6-chloropyridazin-3-yl)pyrrolidin-3-yl]-N-(1-methylcyclopropyl)carbamate (160 mg, 73%) as a solid. LCMS (ES, m/z): 353 [M+H]⁺.

### Synthesis of Intermediate B107

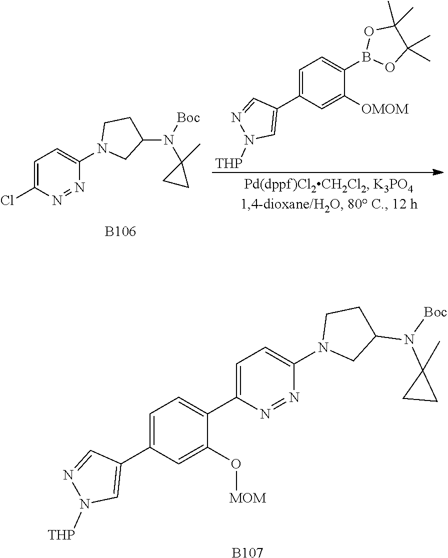

To a solution of tert-butyl N-[1-(6-chloropyridazin-3-yl)pyrrolidin-3-yl]-N-(1-methylcyclopropyl)carbamate (50 mg, 0.142 mmol, 1 equiv) and 4-[3-(methoxymethoxy)-4-(4,4,5,5-tetramethyl-1,3,2-dioxaborolan-2-yl)phenyl]-1-(oxan-2-yl)pyrazole (76.32 mg, 0.185 mmol, 1.3 equiv) in 1,4-dioxane (0.5 mL) and H₂O (0.1 mL) was added K₃PO₄ (90.23 mg, 0.426 mmol, 3 equiv) and Pd(dppf)Cl₂—CH₂Cl₂ (11.54 mg, 0.014 mmol, 0.1 equiv) at room temperature under nitrogen atmosphere. After stirring for 12 hr at 80° C. under nitrogen atmosphere, the resulting mixture was concentrated under reduced pressure. The residue was purified by silica gel column chromatography, eluted with CH₂Cl₂/MeOH (50:1) to afford tert-butyl N-(1-{6-[2-(methoxymethoxy)-4-[1-(oxan-2-yl)pyrazol-4-yl]phenyl]pyridazin-3-yl}pyrrolidin-3-yl)-N-(1-methylcyclopropyl)carbamate (70 mg, 82%) as an oil. LCMS (ES, m/z): 605 [M+H]⁺.

### Synthesis of Compound 107

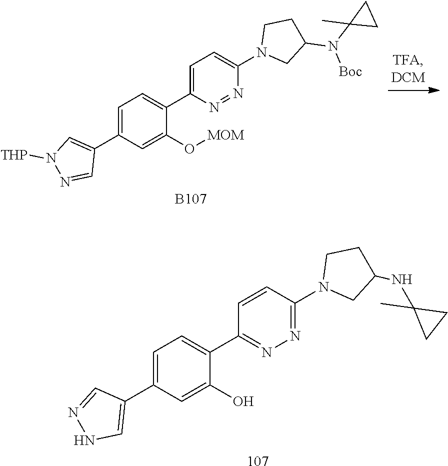

To a stirred solution of tert-butyl N-(1-{6-[2-(methoxymethoxy)-4-[1-(oxan-2-yl)pyrazol-4-yl]phenyl]pyridazin-3-yl}pyrrolidin-3-yl)-N-(1-methylcyclopropyl)carbamate (70 mg, 0.116 mmol, 1 equiv) in DCM (0.7 mL) was added TFA (0.7 mL) at room temperature. The resulting mixture was stirred for 1 hr at room temperature. The resulting mixture was concentrated under reduced pressure. The crude product was purified by reverse phase flash (Condition 5, Gradient 3) to afford 2-(6-{3-[(1-methylcyclopropyl)amino]pyrrolidin-1-yl}pyridazin-3-yl)-5-(1H-pyrazol-4-yl)phenol (22.1 mg, 51%) as a solid. LCMS (ES, m/z): 377[M+H]^{+ 1}H NMR (400 MHz, DMSO-d₆) δ 13.89 (s, 1H), 13.00-12.89 (m, 2H), 8.26-8.10 (m, 3H), 7.84 (d, J=8.2 Hz, 1H), 7.22-7.13 (m, 3H), 3.67 (d, J=54.4 Hz, 3H), 3.49 (d, J=9.0 Hz, 1H), 2.18 (s, 1H), 1.93 (s, 1H), 1.29 (s, 3H), 0.55 (s, 2H), 0.40 (s, 2H).

### Example 40: Synthesis of Compound 112

### Synthesis of Intermediate B108

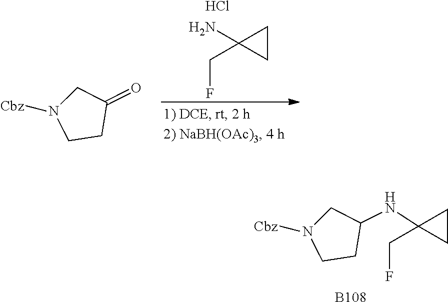

A solution of benzyl 3-oxopyrrolidine-1-carboxylate (500 mg, 2.281 mmol, 1 equiv) and 1-(fluoromethyl)cyclopropan-1-amine hydrochloride (315.01 mg, 2.509 mmol, 1.1 equiv) in 1,2-dichloroethane (10 mL) was stirred for 2 h at room temperature under nitrogen atmosphere. NaBH(OAc)₃ (1450.06 mg, 6.843 mmol, 3 equiv) was added and the mixture was stirred for 4h. The reaction mixture was quenched by water (20 mL) and extracted with DCM (3×30 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (2:1) to afford benzyl 3-{[1-(fluoromethyl)cyclopropyl]amino}pyrrolidine-1-carboxylate (530 mg, 80%) as an oil. LCMS (ES, m/z): 293 [M+H]⁺.

### Synthesis of Intermediate B109

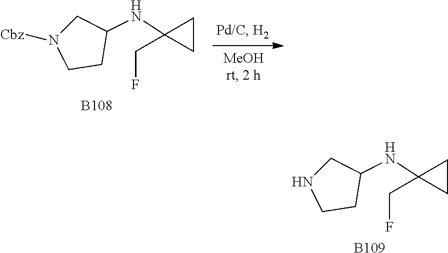

To a solution of benzyl 3-{[1-(fluoromethyl)cyclopropyl]amino}pyrrolidine-1-carboxylate (450 mg, 1.539 mmol, 1 equiv) in methanol (5 mL) was added Pd/C (10%, 200 mg) under nitrogen atmosphere in a 50 mL round-bottom flask. The mixture was hydrogenated at room temperature for 2 h under hydrogen atmosphere using a hydrogen balloon, filtered through a Celite pad and concentrated under reduced pressure. This resulted in N-[1-(fluoromethyl)cyclopropyl]pyrrolidin-3-amine (210 mg, 86%) as an oil. LCMS (ES, m/z): 159 [M+H]⁺.

### Synthesis of Intermediate B110

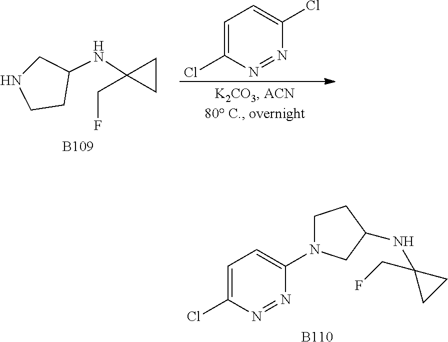

To a stirred solution of N-[1-(fluoromethyl)cyclopropyl]pyrrolidin-3-amine (210 mg, 1.327 mmol, 1 equiv) and 3,6-dichloropyridazine (197.72 mg, 1.327 mmol, 1 equiv) in ACN (2 mL) was added K₂CO₃ (366.87 mg, 2.654 mmol, 2 equiv) at room temperature. The resulting mixture was stirred overnight at 80° C. The resulting mixture was concentrated under reduced pressure. The residue was purified by silica gel column chromatography, eluted with PE/EA (3:1) to afford 1-(6-chloropyridazin-3-yl)-N-[1-(fluoromethyl)cyclopropyl]pyrrolidin-3-amine (200 mg, 56%) as a solid. LCMS (ES, m/z): 271 [M+H]⁺.

### Synthesis of Intermediate B111

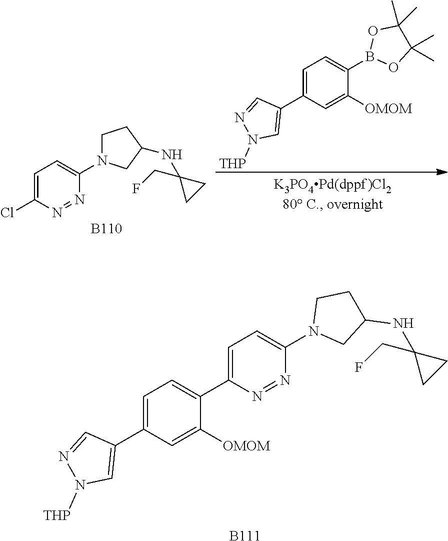

To a stirred mixture of 1-(6-chloropyridazin-3-yl)-N-[1-(fluoromethyl)cyclopropyl]pyrrolidin-3-amine (85 mg, 0.314 mmol, 1 equiv) and 4-[3-(methoxymethoxy)-4-(4,4,5,5-tetramethyl-1,3,2-dioxaborolan-2-yl)phenyl]-1-(oxan-2-yl)pyrazole (156.09 mg, 0.377 mmol, 1.2 equiv) in 1,4-dioxane (1 mL) and H₂O (0.2 mL) was added K₃PO₄ (199.92 mg, 0.942 mmol, 3 equiv) and Pd(dppf)Cl₂ (22.97 mg, 0.031 mmol, 0.1 equiv) at room temperature under nitrogen atmosphere. The resulting mixture was stirred overnight at 80° C. under nitrogen atmosphere. The resulting mixture was concentrated under reduced pressure. The residue was purified by silica gel column chromatography, eluted with PE/EA (2:1) to afford N-[1-(fluoromethyl)cyclopropyl]-1-{6-[2-(methoxymethoxy)-4-[1-(oxan-2-yl)pyrazol-4-yl]phenyl]pyridazin-3-yl}pyrrolidin-3-amine (80 mg, 49%) as an oil. LCMS (ES, m/z): 523 [M+H]⁺.

### Synthesis of Compound 112

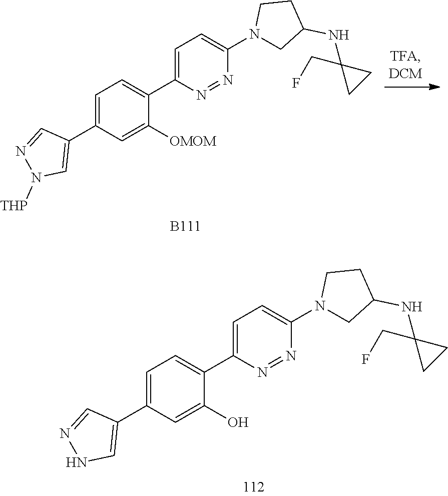

To a stirred solution of N-[1-(fluoromethyl)cyclopropyl]-1-{6-[2-(methoxymethoxy)-4-[1-(oxan-2-yl)pyrazol-4-yl]phenyl]pyridazin-3-yl}pyrrolidin-3-amine (80 mg, 0.153 mmol, 1 equiv) in DCM (1 mL, 102.77 equiv) was added TFA (1 mL, 87.95 equiv) at room temperature. The resulting mixture was stirred for 1 h at room temperature and concentrated under reduced pressure. The crude product was purified by reverse phase flash (Condition 5, Gradient 3) to afford 2-[6-(3-{[1-(fluoromethyl)cyclopropyl]amino}pyrrolidin-1-yl)pyridazin-3-yl]-5-(1H-pyrazol-4-yl)phenol (12.4 mg, 21%) as a solid. LCMS (ES, m/z): 395[M+H]^{+ 1}H NMR (300 MHz, DMSO-d₆) δ 13.92 (s, 1H), 12.98 (s, 1H), 8.21 (d, J=9.8 Hz, 1H), 7.83 (d, J=8.2 Hz, 1H), 7.23-7.09 (m, 3H), 4.49 (s, 1H), 4.32 (s, 1H), 3.72 (d, J=7.1 Hz, 2H), 3.59 (d, J=7.3 Hz, 1H), 3.52-3.43 (m, 1H), 2.18-2.08 (m, 1H), 1.89 (dd, J=12.0, 6.0 Hz, 1H), 0.66-0.59 (m, 4H).

### Example 41: Synthesis of Compound

### Synthesis of Intermediate B112

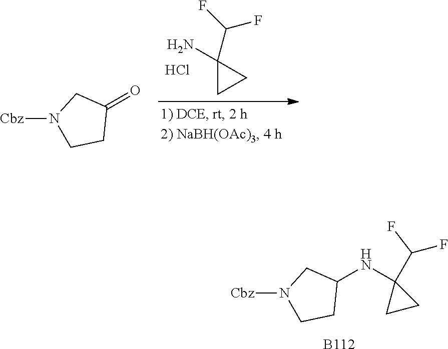

A solution of benzyl 3-oxopyrrolidine-1-carboxylate (500 mg, 2.281 mmol, 1 equiv) and 1-(difluoromethyl)cyclopropan-1-amine hydrochloride (327.40 mg, 2.281 mmol, 1 equiv) in 1,2-dichloroethane (10 mL) was stirred for 2 h at room temperature under nitrogen atmosphere. NaBH(OAc)₃ (1450.06 mg, 6.843 mmol, 3 equiv) was added and the mixture was stirred for 4h. The reaction mixture was quenched by water (20 mL) and extracted with DCM (3*50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (2:1) to afford benzyl 3-{[1-(difluoromethyl)cyclopropyl]amino}pyrrolidine-1-carboxylate (630 mg, 89%) as an oil. LCMS (ES, m/z): 311 [M+H]⁺.

### Synthesis of Intermediate B113

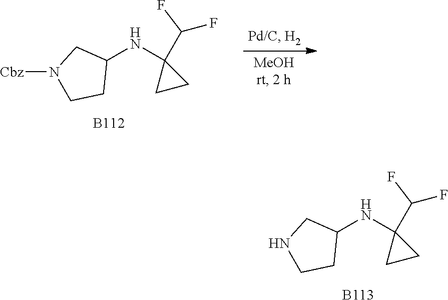

To a solution of benzyl 3-{[1-(difluoromethyl)cyclopropyl]amino}pyrrolidine-1-carboxylate (500 mg, 1.611 mmol, 1 equiv) in methanol (5 mL) was added Pd/C (10%, 200 mg) under nitrogen atmosphere in a 50 mL round-bottom flask. The mixture was hydrogenated at room temperature for 2 hr under hydrogen atmosphere using a hydrogen balloon, filtered through a Celite pad and concentrated under reduced pressure. This resulted in N-[1-(difluoromethyl)cyclopropyl]pyrrolidin-3-amine (250 mg, 88%) as an oil. LCMS (ES, m/z): 177 [M+H]⁺.

### Synthesis of Intermediate B114

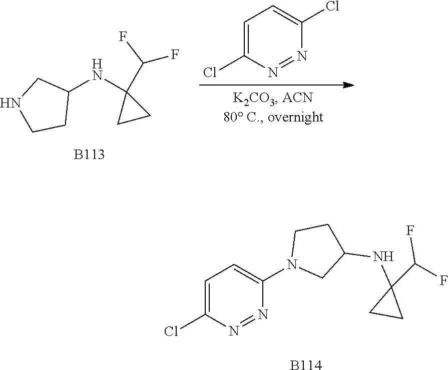

To a stirred solution of N-[1-(difluoromethyl)cyclopropyl]pyrrolidin-3-amine (250 mg, 1.419 mmol, 1 equiv) and 3,6-dichloropyridazine (211.35 mg, 1.419 mmol, 1 equiv) in ACN (2.5 mL) was added K₂CO₃ (392.16 mg, 2.838 mmol, 2 equiv) at room temperature. The resulting mixture was stirred overnight at 80° C. The resulting mixture was concentrated under reduced pressure. The residue was purified by silica gel column chromatography, eluted with PE/EA (3:1) to afford 1-(6-chloropyridazin-3-yl)-N-[1-(difluoromethyl)cyclopropyl]pyrrolidin-3-amine (240 mg, 59%) as a solid. LCMS (ES, m/z): 289 [M+H]⁺.

### Synthesis of B115

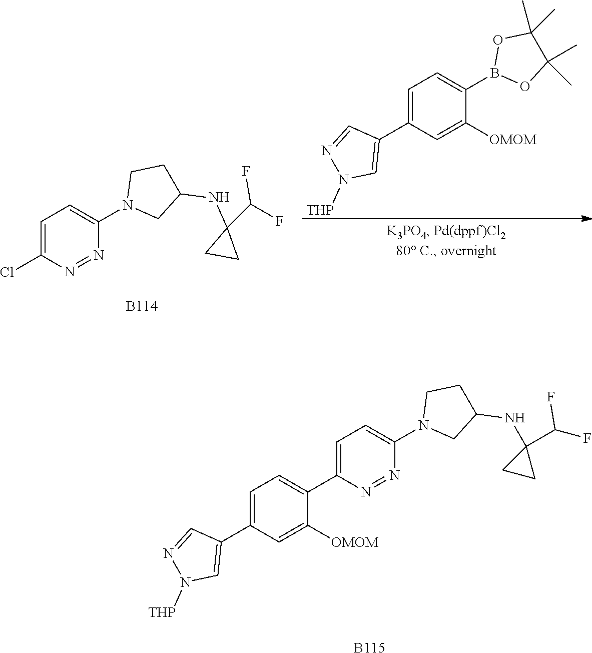

To a stirred mixture of 1-(6-chloropyridazin-3-yl)-N-[1-(difluoromethyl)cyclopropyl]pyrrolidin-3-amine (85 mg, 0.294 mmol, 1 equiv) and 4-[3-(methoxymethoxy)-4-(4,4,5,5-tetramethyl-1,3,2-dioxaborolan-2-yl)phenyl]-1-(oxan-2-yl)pyrazole (146.36 mg, 0.353 mmol, 1.2 equiv) in 1,4-dioxane (1 mL) and H₂O (0.2 mL) was added K₃PO₄ (187.47 mg, 0.882 mmol, 3 equiv) and Pd(dppf)Cl₂ (21.54 mg, 0.029 mmol, 0.1 equiv) at room temperature under nitrogen atmosphere. The resulting mixture was stirred overnight at 80° C. under nitrogen atmosphere and concentrated under reduced pressure. The residue was purified by silica gel column chromatography, eluted with PE/EA (2:1) to afford N-[1-(difluoromethyl)cyclopropyl]-1-{6-[2-(methoxymethoxy)-4-[1-(oxan-2-yl)pyrazol-4-yl]phenyl]pyridazin-3-yl}pyrrolidin-3-amine (80 mg, 50%) as an oil. LCMS (ES, m/z): 541 [M+H]⁺.

### Synthesis of Compound 117

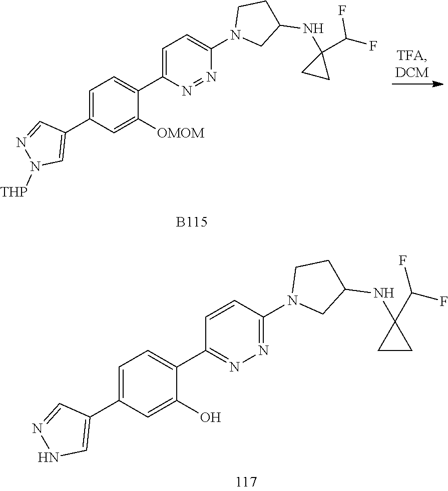

To a stirred solution of N-[1-(difluoromethyl)cyclopropyl]-1-{6-[2-(methoxymethoxy)-4-[1-(oxan-2-yl)pyrazol-4-yl]phenyl]pyridazin-3-yl}pyrrolidin-3-amine (85 mg, 0.157 mmol, 1 equiv) in DCM (1 mL, 15.731 mmol, 100.05 equiv) was added TFA (1 mL, 13.463 mmol, 85.63 equiv) at room temperature. The resulting mixture was stirred for 1 h at room temperature and concentrated under reduced pressure. The crude product was purified by reverse phase flash (Condition 5, Gradient 3) to afford 2-[6-(3-{[1-(difluoromethyl)cyclopropyl]amino}pyrrolidin-1-yl)pyridazin-3-yl]-5-(1H-pyrazol-4-yl)phenol (13.8 mg, 21%) as a solid. LCMS (ES, m/z): 413[M+H]⁺.

¹H NMR (300 MHz, DMSO-d₆) δ 13.91 (s, 1H), 12.98 (s, 1H), 8.29-8.16 (m, 2H), 7.98 (s, 1H), 7.83 (d, J=8.1 Hz, 1H), 7.23-7.09 (m, 3H), 3.70 (d, J=7.7 Hz, 2H), 3.60 (s, 1H), 3.46 (s, 1H), 2.95 (d, J=4.2 Hz, 1H), 2.13 (s, 1H), 1.89 (s, 1H), 0.79 (d, J=4.5 Hz, 2H), 0.75 (s, 2H).

### Example 42: Synthesis of Compound 110

### Synthesis of Intermediate B116

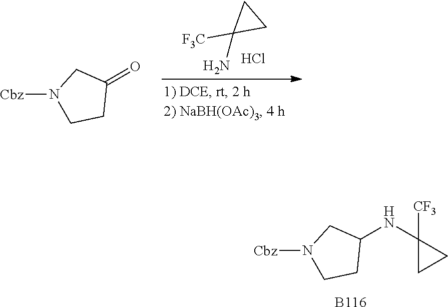

A mixture of benzyl 3-oxopyrrolidine-1-carboxylate (500 mg, 2.281 mmol, 1 equiv) and 1-(trifluoromethyl)cyclopropan-1-amine hydrochloride (442 mg, 2.737 mmol, 1.2 equiv) in 1,2-dichloroethane (10 mL) was stirred for 2 hr at room temperature. NaBH(OAc)₃ (1450 mg, 6.843 mmol, 3 equiv) was added and the mixture was stirred for 4 hr. The reaction mixture was quenched by water and extracted with DCM (3×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (2:1) to afford benzyl 3-{[1-(trifluoromethyl)cyclopropyl]amino}pyrrolidine-1-carboxylate (560 mg, 75%) as an oil. LCMS (ES, m/z): 329 [M+H]⁺.

### Synthesis of Intermediate B117

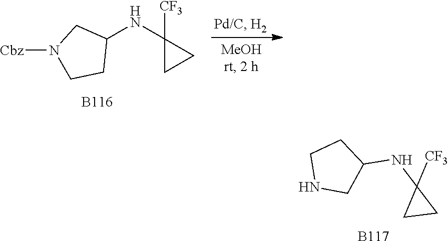 [SMILES: "B116" ; O=C(OCc1ccccc1)N1CCC(NC2(C(F)(F)F)CC2)C1 ; "B117" ; FC(F)(F)C1(NC2CCNC2)CC1]

To a solution of benzyl 3-{[1-(trifluoromethyl)cyclopropyl]amino}pyrrolidine-1-carboxylate (500 mg, 1.523 mmol, 1 equiv) in methanol (10 mL) was added Pd/C (10%, 200 mg) under nitrogen atmosphere in a 50 mL round-bottom flask. The mixture was hydrogenated at room temperature for 2 hr under hydrogen atmosphere using a hydrogen balloon, filtered through a Celite pad and concentrated under reduced pressure. This resulted in N-[1-(trifluoromethyl)cyclopropyl]pyrrolidin-3-amine (280 mg, 95%) as an oil. LCMS (ES, m/z): 195 [M+H]⁺.

### Synthesis of Intermediate B118

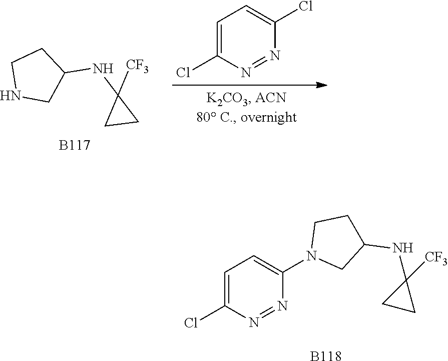

To a stirred solution of N-[1-(trifluoromethyl)cyclopropyl]pyrrolidin-3-amine (280 mg, 1.442 mmol, 1 equiv) and pyridazine, 3,6-dichloro- (236.26 mg, 1.586 mmol, 1.1 equiv) in ACN (5 mL) was added K₂CO₃ (398.53 mg, 2.884 mmol, 2 equiv) at room temperature. The resulting mixture was stirred overnight at 80° C. The resulting mixture was filtered and the filtrate was concentrated under reduced pressure. The residue was purified by silica gel column chromatography, eluted with PE/EA (3:1) to afford 1-(6-chloropyridazin-3-yl)-N-[1-(fluoromethyl)cyclopropyl]pyrrolidin-3-amine (200 mg, 56%) as a solid. LCMS (ES, m/z): 307 [M+H]⁺.

### Synthesis of Intermediate B119

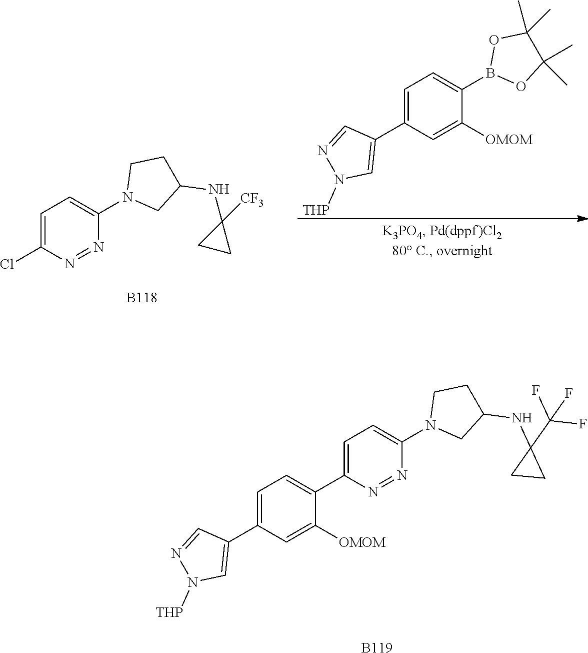

To a solution of 1-(6-chloropyridazin-3-yl)-N-[1-(trifluoromethyl)cyclopropyl]pyrrolidin-3-amine (100 mg, 0.326 mmol, 1 equiv) and 4-[3-(methoxymethoxy)-4-(4,4,5,5-tetramethyl-1,3,2-dioxaborolan-2-yl)phenyl]-1-(oxan-2-yl)pyrazole (162 mg, 0.391 mmol, 1.2 equiv) in 1,4-dioxane (0.9 mL) and H₂O (0.3 mL) were added K₃PO₄ (208 mg, 0.978 mmol, 3 equiv) and Pd(dppf)Cl₂ (24 mg, 0.033 mmol, 0.1 equiv). After stirring overnight at 80° C. under a nitrogen atmosphere, the resulting mixture was concentrated under reduced pressure. The residue was purified by silica gel column chromatography, eluted with PE/EA (2:1) to afford 1-{6-[2-(methoxymethoxy)-4-[1-(oxan-2-yl)pyrazol-4-yl]phenyl]pyridazin-3-yl}-N-[1-(trifluoromethyl) cyclopropyl]pyrrolidin-3-amine (140 mg, 77%) as a solid. LCMS (ES, m/z): 559 [M+H]⁺.

### Synthesis of Compound 110

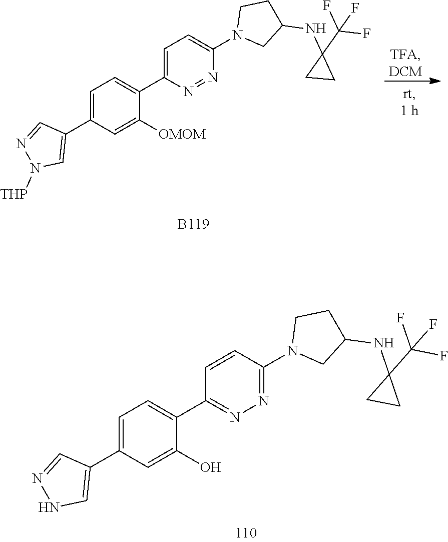

To a stirred solution of 1-{6-[2-(methoxymethoxy)-4-[1-(oxan-2-yl)pyrazol-4-yl]phenyl]pyridazin-3-yl}-N-[1-(trifluoromethyl)cyclopropyl]pyrrolidin-3-amine (140 mg, 0.251 mmol, 1 equiv) in DCM (1 mL) was added TFA (1 mL, 13.463 mmol, 53.72 equiv) at room temperature. The resulting mixture was stirred for 1 h at room temperature. The resulting mixture was concentrated under reduced pressure. The crude product was purified by reverse phase flash (Condition 5, Gradient 3) to afford 5-(1H-pyrazol-4-yl)-2-[6-(3-{[1-(trifluoromethyl)cyclopropyl]amino}pyrrolidin-1-yl)pyridazin-3-yl]phenol (12.2 mg, 11%) as a solid. LCMS (ES, m/z): 431[M+H]^{+ 1}H NMR (300 MHz, DMSO-d₆) δ 13.90 (s, 1H), 12.97 (s, 1H), 8.21 (d, J=9.8 Hz, 1H), 7.84 (d, J=8.2 Hz, 1H), 7.23-7.10 (m, 3H), 3.76-3.58 (m, 3H), 3.52 (dd, J=15.3, 8.3 Hz, 1H), 3.29 (s, 2H), 2.20-2.09 (m, 1H), 1.89 (dd, J=12.5, 6.3 Hz, 1H), 1.02 (s, 2H), 0.94 (s, 2H).

### Example 43: Synthesis of Compound 111

### Synthesis of Intermediate B120

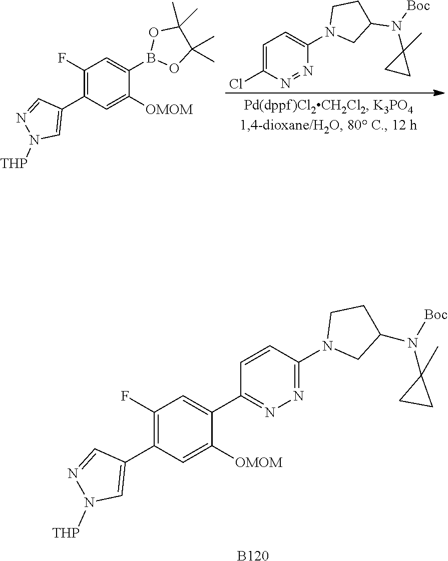

To a solution of tert-butyl N-[1-(6-chloropyridazin-3-yl)pyrrolidin-3-yl]-N-(1-methylcyclopropyl)carbamate (50 mg, 0.142 mmol, 1 equiv) and 4-[2-fluoro-5-(methoxymethoxy)-4-(4,4,5,5-tetramethyl-1,3,2-dioxaborolan-2-yl)phenyl]-1-(oxan-2-yl)pyrazole (79.63 mg, 0.185 mmol, 1.3 equiv) in 1,4-dioxane (0.5 mL) and H₂O (0.1 mL) was added K₃PO₄ (90.23 mg, 0.426 mmol, 3 equiv) and Pd(dppf)Cl₂—CH₂Cl₂ (11.54 mg, 0.014 mmol, 0.1 equiv) at room temperature under nitrogen atmosphere. After stirring for 12 hr at 80° C. under nitrogen atmosphere, the resulting mixture was concentrated under reduced pressure. The residue was purified by silica gel column chromatography, eluted with CH₂Cl₂/MeOH (50:1) to afford tert-butyl N-(1-{6-[5-fluoro-2-(methoxymethoxy)-4-[1-(oxan-2-yl)pyrazol-4-yl]phenyl]pyridazin-3-yl}pyrrolidin-3-yl)-N-(1-methylcyclopropyl)carbamate (60 mg, 68%) as an oil. LCMS (ES, m/z): 623 [M+H]⁺.

### Synthesis of Compound 111

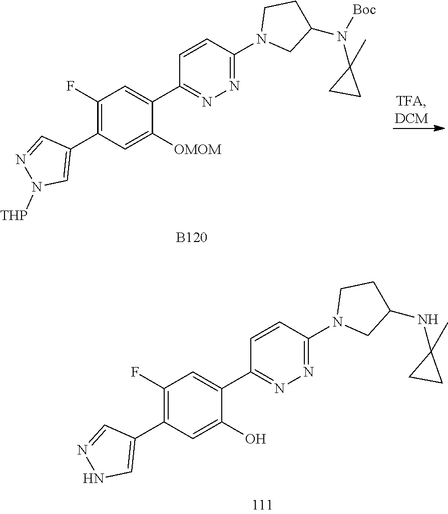

To a stirred solution of tert-butyl N-(1-{6-[5-fluoro-2-(methoxymethoxy)-4-[1-(oxan-2-yl)pyrazol-4-yl]phenyl]pyridazin-3-yl}pyrrolidin-3-yl)-N-(1-methylcyclopropyl)carbamate (60 mg, 0.096 mmol, 1 equiv) in DCM (0.6 mL) was added TFA (0.6 mL) at room temperature. The resulting mixture was stirred for 1 h at room temperature. The resulting mixture was concentrated under reduced pressure. The crude product was purified by reverse phase flash chromatography (Condition 5, Gradient 3) to afford 4-fluoro-2-(6-{3-[(1-methylcyclopropyl)amino]pyrrolidin-1-yl}pyridazin-3-yl)-5-(1H-pyrazol-4-yl)phenol (9 mg, 24%) as a solid. LCMS (ES, m/z): 395[M+H]^{+ 1}H NMR (400 MHz, DMSO-d₆) δ 8.53 (d, J=2.1 Hz, 1H), 8.22 (dd, J=9.9, 1.9 Hz, 1H), 8.10 (d, J=2.2 Hz, 2H), 7.80 (dd, J=12.7, 1.9 Hz, 1H), 7.29 (dd, J=7.0, 2.0 Hz, 1H), 7.14 (dd, J=9.7, 2.0 Hz, 1H), 3.45 (s, 4H), 2.48 (s, 1H), 2.13 (dd, J=12.3, 6.2 Hz, 1H), 1.87 (dd, J=12.6, 6.6 Hz, 1H), 1.31-1.22 (m, 4H), 0.52-0.42 (m, 2H), 0.38-0.28 (m, 2H).

### Example 44: Synthesis of Compound 108

### Synthesis of Intermediate B121

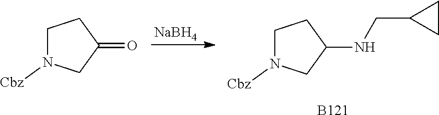

A solution of benzyl 3-oxopyrrolidine-1-carboxylate (10 g, 45.612 mmol, 1 equiv) and 1-cyclopropylmethanamine (6.49 g, 91.224 mmol, 2 equiv) in DCM (100 mL) was stirred for 2 h at room temperature. NaBH₄ (3.45 g, 91.224 mmol, 2 equiv) was added and the mixture was stirred for 4 h. The reaction mixture was quenched by water and extracted with DCM (3×50 mL). The combined organic layers were washed with brine (1×100 mL), dried over anhydrous Na2SO4. After filtration, the filtrate was concentrated under reduced pressure. The residue was purified by silica gel column chromatography, eluted with PE/EA (1:1) to afford benzyl 3-[(cyclopropylmethyl)amino]pyrrolidine-1-carboxylate (8.6 g, 69%) as an oil. LCMS (ES, m/z): 275 [M+H]⁺.

### Synthesis of Intermediate B122

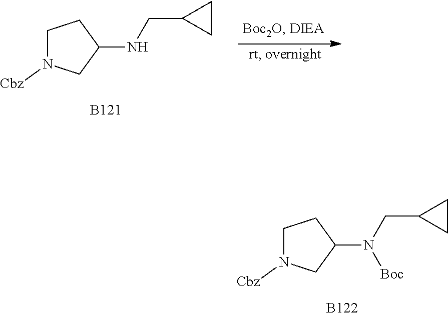

To a stirred solution of benzyl 3-[(cyclopropylmethyl)amino]pyrrolidine-1-carboxylate (8.6 g, 31.345 mmol, 1 equiv) and DIEA (6.08 g, 47.017 mmol, 1.5 equiv) in DCM (100 mL) was added Boc₂O (8.21 g, 37.614 mmol, 1.2 equiv) in portions at room temperature. The resulting mixture was stirred overnight at room temperature. The resulting mixture was concentrated under vacuum. The residue was purified by silica gel column chromatography, eluted with PE/EA (2:1) to afford benzyl 3-[(tert-butoxycarbonyl)(cyclopropylmethyl)amino]pyrrolidine-1-carboxylate (10 g, 85%) as an oil. LCMS (ES, m/z): 375 [M+H]⁺.

### Synthesis of Intermediate B123

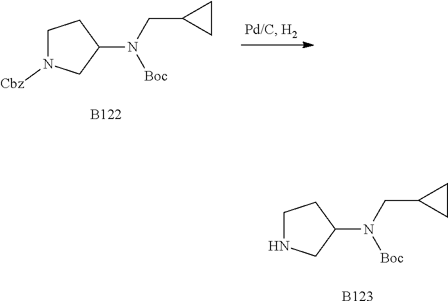

To a solution of benzyl 3-[(tert-butoxycarbonyl)(cyclopropylmethyl)amino]pyrrolidine-1-carboxylate (10 g, 26.704 mmol, 1 equiv) in 100 mL MeOH was added Pd/C (10%, 1 g) under nitrogen atmosphere in a 250 mL round-bottom flask. The mixture was hydrogenated at room temperature for 2 hr under hydrogen atmosphere using a hydrogen balloon. The mixture was filtered through a Celite pad and concentrated under reduced pressure to afford tert-butyl N-(cyclopropylmethyl)-N-(pyrrolidin-3-yl)carbamate (5.5 g, 86%) as an oil. The crude product was used in the next step directly without further purification. LCMS (ES, m/z): 241 [M+H]⁺.

### Synthesis of Intermediate B124

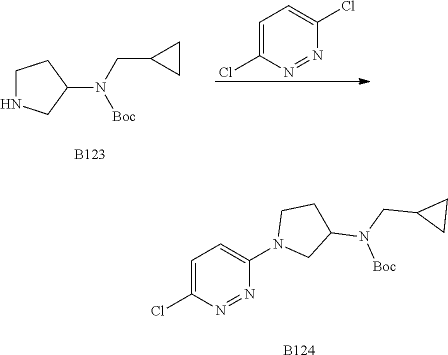

To a stirred mixture of tert-butyl N-(cyclopropylmethyl)-N-(pyrrolidin-3-yl)carbamate (1.5 g, 6.241 mmol, 1 equiv) and pyridazine, 3,6-dichloro- (1.12 g, 7.489 mmol, 1.2 equiv) in ACN (15 mL) was added K₂CO₃ (2.59 g, 18.723 mmol, 3 equiv) at room temperature. The resulting mixture was stirred for 12 hr at 80° C. The resulting mixture was filtered and the filtrate was concentrated under reduced pressure. The residue was purified by silica gel column chromatography, eluted with PE/EA (4:1) to afford tert-butyl N-[1-(6-chloropyridazin-3-yl)pyrrolidin-3-yl]-N-(cyclopropylmethyl)carbamate (1.6 g, 73%) as a solid. LCMS (ES, m/z): 353 [M+H]⁺.

### Synthesis of Intermediate B125

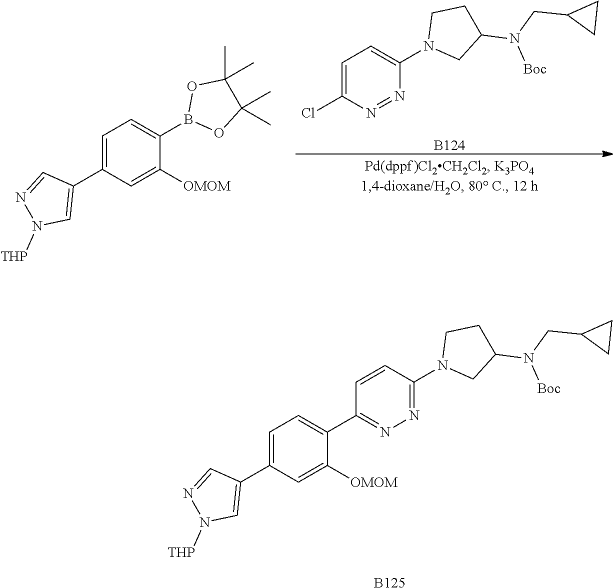

To a solution of tert-butyl N-[1-(6-chloropyridazin-3-yl)pyrrolidin-3-yl]-N-(cyclopropylmethyl)carbamate (50 mg, 0.142 mmol, 1 equiv) and 4-[3-(methoxymethoxy)-4-(4,4,5,5-tetramethyl-1,3,2-dioxaborolan-2-yl)phenyl]-1-(oxan-2-yl)pyrazole (76.32 mg, 0.185 mmol, 1.3 equiv) in 1,4-dioxane (0.5 mL) and H₂O (0.1 mL) were added K₃PO₄ (90.23 mg, 0.426 mmol, 3 equiv) and Pd(dppf)Cl₂—CH₂Cl₂ (11.54 mg, 0.014 mmol, 0.1 equiv) at room temperature under nitrogen atmosphere. After stirring for 12 hr at 80° C. under nitrogen atmosphere, the resulting mixture was concentrated under reduced pressure. The residue was purified by silica gel column chromatography, eluted with CH₂Cl₂/MeOH (50:1) to afford tert-butyl N-(cyclopropylmethyl)-N-(1-{6-[2-(methoxymethoxy)-4-[1-(oxan-2-yl)pyrazol-4-yl]phenyl]pyridazin-3-yl}pyrrolidin-3-yl)carbamate (65 mg, 76%) as an oil. LCMS (ES, m/z): 605 [M+H]⁺.

### Synthesis of Compound 108

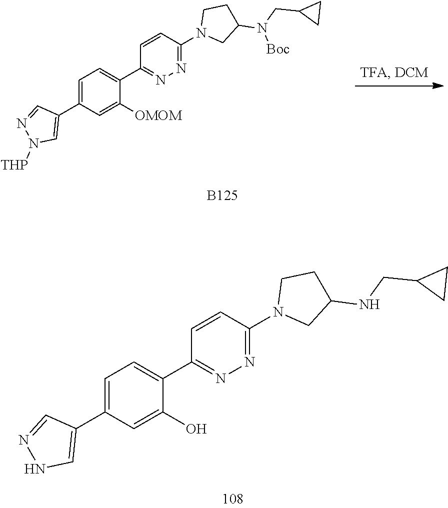

To a stirred solution of tert-butyl N-(cyclopropylmethyl)-N-(1-{6-[2-(methoxymethoxy)-4-[1-(oxan-2-yl)pyrazol-4-yl]phenyl]pyridazin-3-yl}pyrrolidin-3-yl)carbamate (65 mg, 0.107 mmol, 1 equiv) in DCM (0.65 mL) was added TFA (0.65 mL) at room temperature. The resulting mixture was stirred for 1 hr at room temperature. The resulting mixture was concentrated under reduced pressure. The crude product was purified by reverse phase flash (Condition 5, Gradient 3) to afford 2-(6-{3-[(cyclopropylmethyl)amino]pyrrolidin-1-yl}pyridazin-3-yl)-5-(1H-pyrazol-4-yl)phenol; methane (16.3 mg, 39%) as a solid. LCMS (ES, m/z): 377[M+H]^{+ 1}H NMR (400 MHz, DMSO-d₆) δ 13.92 (s, 1H), 12.97 (s, 1H), 8.26 (s, 1H), 8.21 (d, J=9.8 Hz, 1H), 7.98 (s, 1H), 7.84 (d, J=8.2 Hz, 1H), 7.22-7.12 (m, 3H), 3.68 (dd, J=10.7, 5.9 Hz, 1H), 3.61 (t, J=7.7 Hz, 1H), 3.50 (dt, J=22.6, 6.2 Hz, 2H), 2.47 (d, J=6.8 Hz, 4H), 2.14 (dq, J=12.9, 6.6 Hz, 1H), 1.88 (dq, J=12.7, 6.2 Hz, 1H), 0.93-0.86 (m, 1H), 0.47-0.38 (m, 2H), 0.17-0.09 (m, 2H).

### Example 45: Synthesis of Compound 109

### Synthesis of Intermediate B126

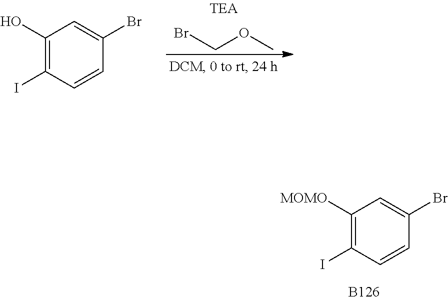

Into a 100 mL 3-necked round-bottom flask were added 5-bromo-2-iodophenol (3.0 g, 10.037 mmol, 1.00 equiv), DCM (30 mL) and TEA (3.05 g, 30.111 mmol, 3.0 equiv) at room temperature. To the above mixture was added bromo(methoxy)methane (2.51 g, 20.074 mmol, 2.0 equiv) dropwise at 0° C. The resulting mixture was stirred for 24 h at room temperature. The resulting mixture was diluted with CH₂Cl₂ (150 mL), washed with water (2×20 mL). The organic phase was dried over anhydrous Na₂SO₄. After filtration, the filtrate was concentrated under reduced pressure. The residue was purified by silica gel column chromatography, eluted with PE/EA (9:1) to afford 4-bromo-1-iodo-2-(methoxymethoxy)benzene (1.7 g, 49.39%) as an oil. LCMS (ES, m/z): 343 [M+H]⁺.

### Synthesis of Intermediate B127

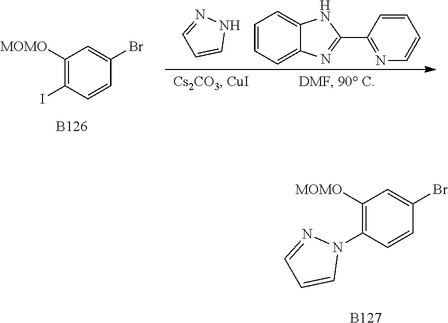

Into a 40 mL vial were added 4-bromo-1-iodo-2-(methoxymethoxy)benzene (300 mg, 0.875 mmol, 1.00 equiv), DMF (3 mL), 1H-pyrazole (59.55 mg, 0.875 mmol, 1.0 equiv),Cs₂CO₃ (570.02 mg, 1.750 mmol, 2.0 equiv), 2-(pyridin-2-yl)-1H-1,3-benzodiazole (17.08 mg, 0.088 mmol, 0.1 equiv) and CuI (16.66 mg, 0.088 mmol, 0.1 equiv) at room temperature under nitrogen atmosphere. The resulting mixture was stirred overnight at 90° C. under nitrogen atmosphere. The mixture was diluted with water (20 mL), extracted with EtOAc (3×20 mL).The combined organic layers were washed with brine (2×20 mL), dried over anhydrous Na₂SO₄. After filtration, the filtrate was concentrated under reduced pressure. The residue was purified by silica gel column chromatography, eluted with PE/EA (3:1) to afford 1-[4-bromo-2-(methoxymethoxy)phenyl]pyrazole (120 mg, 48%) as an oil. LCMS (ES, m/z): 283 [M+H]⁺.

### Synthesis of Intermediate B128

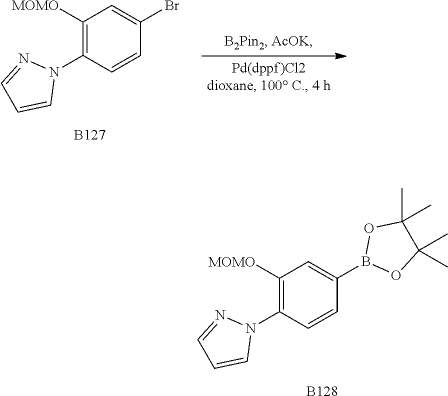

Into an 8 mL vial were added 1-[4-bromo-2-(methoxymethoxy)phenyl]pyrazole (120 mg, 0.424 mmol, 1.00 equiv), 1,4-dioxane (2 mL), 4,4,5,5-tetramethyl-2-(tetramethyl-1,3,2-dioxaborolan-2-yl)-1,3,2- dioxaborolane (129.16 mg, 0.509 mmol, 1.2 equiv), AcOK (124.79 mg, 1.272 mmol, 3.0 equiv) and Pd(dppf)Cl₂ (31.01 mg, 0.042 mmol, 0.1 equiv) at room temperature under nitrogen atmosphere. The resulting mixture was stirred for 4 h at 100° C. under nitrogen atmosphere. The mixture was concentrated under reduced pressure. The residue was purified by silica gel column chromatography, eluted with PE/EA (3:1) to afford 1-[2-(methoxymethoxy)-4-(4,4,5,5-tetramethyl-1,3,2-dioxaborolan-2-yl)phenyl]pyrazole (110 mg, 79%) as an oil. LCMS (ES, m/z): 331 [M+H]⁺.

### Synthesis of Intermediate B129

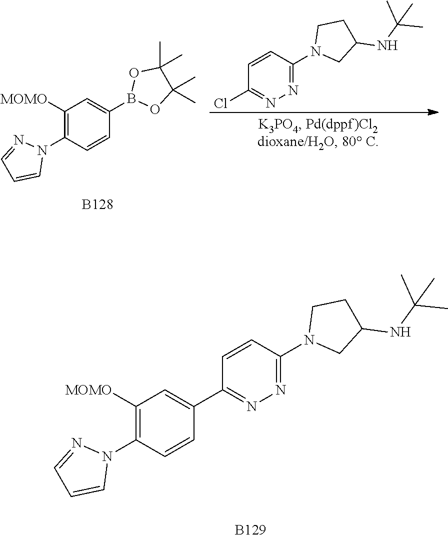

Into an 8 mL vial were added 1-[2-(methoxymethoxy)-4-(4,4,5,5-tetramethyl-1,3,2-dioxaborolan-2-yl) phenyl]pyrazole (90 mg, 0.273 mmol, 1.0 equiv), dioxane (1 mL), water (0.2 mL), N-tert-butyl-1-(6-chloropyridazin-3-yl)pyrrolidin-3-amine (69.44 mg, 0.273 mmol, 1.0 equiv), K₃PO₄ (115.71 mg, 0.546 mmol, 2.0 equiv) and Pd(dppf)Cl₂ (22.20 mg, 0.027 mmol, 0.1 equiv) at room temperature under nitrogen atmosphere. The resulting mixture was stirred overnight at 8° C. under nitrogen atmosphere. The mixture was diluted with water (1OmL) and extracted with EtOAc (3×10 mL). The combined organic layers were washed with brine (1×20 mL), dried over anhydrous Na₂SO₄. After filtration, the filtrate was concentrated under reduced pressure. The residue was purified by silica gel column chromatography, eluted with CH₂Cl₂/MeOH (9:1) to afford N-tert-butyl-1-{6-[3-(methoxymethoxy)-4-(pyrazol-1-yl)phenyl]pyridazin-3-yl}pyrrolidin-3-amine (60 mg, 52%) as an oil. LCMS (ES, m/z): 423 [M+H]⁺.

### Synthesis of Compound 109

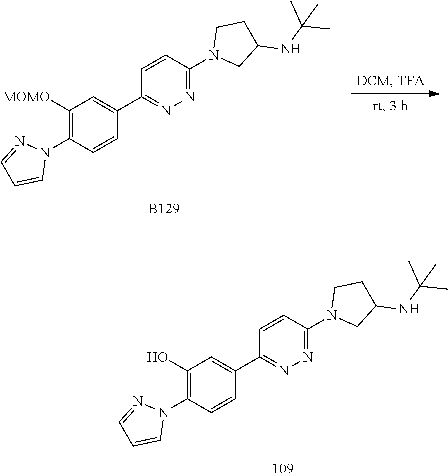

Into an 8 mL vial were added N-tert-butyl-1-{6-[3-(methoxymethoxy)-4-(pyrazol-1-yl)phenyl]pyridazin-3-yl}pyrrolidin-3-amine (60 mg, 0.142 mmol, 1 equiv), DCM (1 mL, 15.731 mmol, 110.78 equiv) and TFA (1 mL, 13.463 mmol, 94.81 equiv) at room temperature. The resulting mixture was stirred for 3 h at room temperature. The resulting mixture was concentrated under reduced pressure. The residue was purified by reverse phase flash (Condition 4, Gradient 3) to afford 5-{6-[3-(tert-butylamino)pyrrolidin-1-yl]pyridazin-3-yl}-2-(pyrazol-1-yl)phenol trifluoroacetate (12 mg, 18%) as a solid. LCMS (ES, m/z): 379 [M+H]^{+ 1}H NMR (300 MHz, DMSO-d6) δ 11.06 (s, 1H), 9.03 (s, 1H), 8.91 (d, J=11.8 Hz, 1H), 8.50 (d, J=2.5 Hz, 1H), 8.19 (d, J=9.6 Hz, 1H), 7.85 (d, J=8.4 Hz, 1H), 7.79 (d, J=1.9 Hz, 2H), 7.60 (dd, J=8.6, 1.9 Hz, 1H), 7.44 (d, J=9.6 Hz, 1H), 6.57 (t, J=2.1 Hz, 1H), 4.21 (s, 1H), 4.04 (dd, J=11.6, 6.6 Hz, 1H), 3.77 (dd, J=11.3, 5.5 Hz, 2H), 3.63 (d, J=9.5 Hz, 1H), 2.29 (dd, J=13.4, 6.9 Hz, 1H), 1.38 (s, 9H).

### Example 46: Synthesis of Compound 113

### Synthesis of Intermediate B130

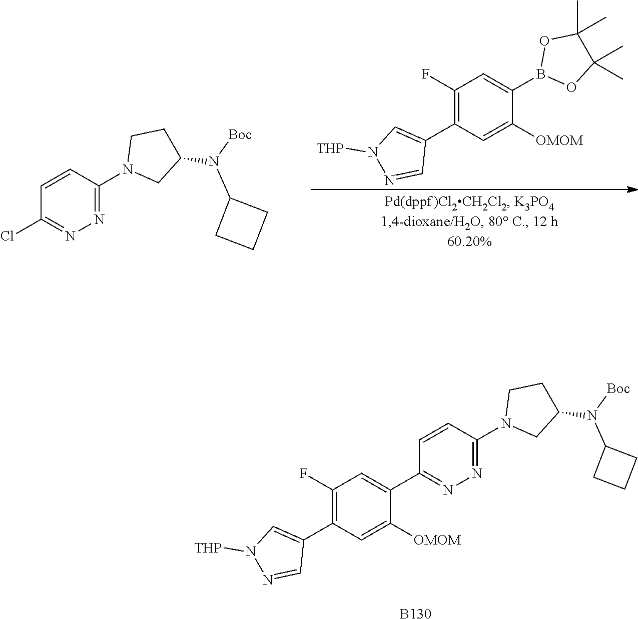

To a solution of tert-butyl N-[(3S)-1-(6-chloropyridazin-3-yl)pyrrolidin-3-yl]-N-cyclobutylcarbamate (160 mg, 0.453 mmol, 1 equiv) and 4-[2-fluoro-5-(methoxymethoxy)-4-(4,4,5,5-tetramethyl-1,3,2-dioxaborolan-2-yl)phenyl]-1-(oxan-2-yl)pyrazole (235.23 mg, 0.544 mmol, 1.2 equiv) in 1,4-dioxane (1.5 mL) and H₂O (0.3 mL) was added K₃PO₄ (288.75 mg, 1.359 mmol, 3 equiv) and Pd(dppf)Cl₂—CH₂Cl₂(36.94 mg, 0.045 mmol, 0.1 equiv) at room temperature under nitrogen atmosphere. After stirring for 12 hr at 80° C. under nitrogen atmosphere, the resulting mixture was concentrated under reduced pressure. The residue was purified by silica gel column chromatography, eluted with CH₂Cl₂/MeOH (50:1) to afford tert-butyl N-cyclobutyl-N-[(3S)-1-{6-[5-fluoro-2-(methoxymethoxy)-4-[1-(oxan-2-yl)pyrazol-4-yl]phenyl]pyridazin-3-yl}pyrrolidin-3-yl]carbamate (170 mg, 60%) as an oil. LCMS (ES, m/z): 623 [M+H]⁺.

### Synthesis of Compound 113

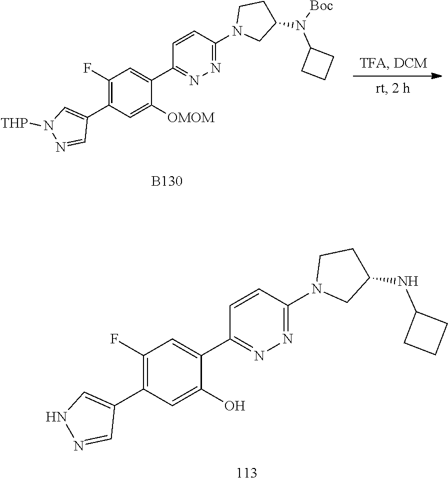

To a stirred solution of tert-butyl N-cyclobutyl-N-[(3S)-1-{6-[5-fluoro-2-(methoxymethoxy)-4-[1-(oxan-2-yl)pyrazol-4-yl]phenyl]pyridazin-3-yl}pyrrolidin-3-yl]carbamate (170 mg, 0.273 mmol, 1 equiv) in DCM (2 mL) was added TFA (2 mL, 26.926 mmol, 85.70 equiv) at room temperature. The resulting mixture was stirred for 1 h at room temperature and concentrated under reduced pressure. The crude product was purified by reverse phase flash (Condition 5, Gradient 3) to afford 2-{6-[(3S)-3-(cyclobutylamino)pyrrolidin-1-yl]pyridazin-3-yl}-4-fluoro-5-(1H-pyrazol-4-yl)phenol (67.6 mg, 63%) as a solid. LCMS (ES, m/z): 395[M+H]^{+ 1}H NMR (300 MHz, DMSO-d₆) δ 13.65 (s, 1H), 13.12 (s, 1H), 8.23 (d, J=9.8 Hz, 1H), 7.99 (s, 1H), 7.81 (d, J=12.7 Hz, 1H), 7.29 (d, J=7.0 Hz, 1H), 7.14 (d, J=9.8 Hz, 1H), 3.63 (dt, J=11.6, 6.0 Hz, 2H), 3.50 (t, J=7.8 Hz, 1H), 3.39 (t, J=5.6 Hz, 1H), 3.31-3.20 (m, 2H), 2.10 (dt, J=13.0, 6.0 Hz, 4H), 1.81 (dt, J=13.3, 6.8 Hz, 1H), 1.73-1.49 (m, 4H).

### Example 47: Synthesis of Compound 114

### Synthesis of Intermediate B131

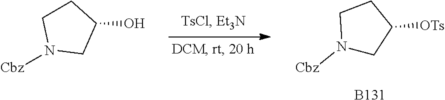 [SMILES: O=C(OCc1ccccc1)N1CC[C@H](O)C1 ; "B131" ; Cc1ccc(S(=O)(=O)O[C@H]2CCN(C(=O)OCc3ccccc3)C2)cc1]

To a stirred solution of benzyl (3S)-3-hydroxypyrrolidine-1-carboxylate (10 g, 45.197 mmol, 1 equiv) and TEA (9.15 g, 90.394 mmol, 2 equiv) in DCM (100 mL) was added TsCl (17.23 g, 90.394 mmol, 2 equiv) in portions at room temperature. The resulting mixture was stirred overnight at room temperature. The reaction solution was washed with 2×50 mL of brine and dried over anhydrous Na₂SO₄. After filtration, the filtrate was concentrated under reduced pressure to afford benzyl (3S)-3-[(4-methylbenzenesulfonyl)oxy]pyrrolidine-1-carboxylate (8.1 g, 48%) as an oil. LCMS (ES, m/z): 376 [M+H]⁺.

### Synthesis of Intermediate

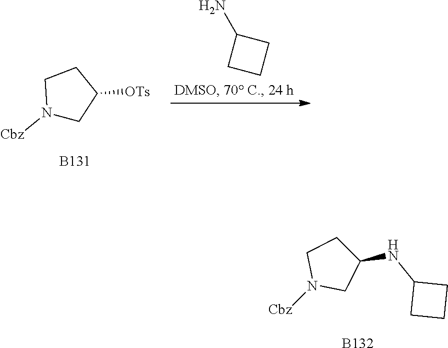

A solution of benzyl (3S)-3-[(4-methylbenzenesulfonyl)oxy]pyrrolidine-1-carboxylate (8.1 g, 21.575 mmol, 1 equiv) in DMSO (40 mL) was added cyclobutylamine (7.67 g, 107.875 mmol, 5 equiv) at room temperature. The resulting mixture was stirred for 24 hr at 70° C. The resulting mixture was diluted with H₂O (150 mL) and extracted with EtOAc (3×100 mL). The combined organic layers were washed with brine (3×100 mL), dried over anhydrous Na₂SO₄. After filtration, the filtrate was concentrated under reduced pressure to afford benzyl (3R)-3-(cyclobutylamino)pyrrolidine-1-carboxylate (4.1 g, 69%) as a solid. LCMS (ES, m/z): 275 [M+H]⁺.

### Synthesis of Intermediate B133

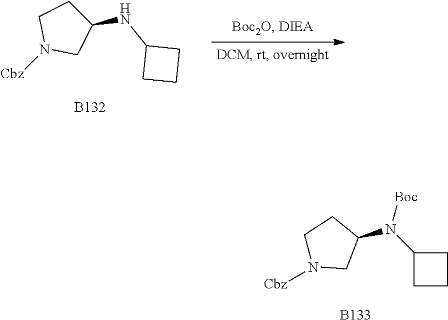

To a stirred solution of benzyl (3R)-3-(cyclobutylamino)pyrrolidine-1-carboxylate (4.1 g, 14.944 mmol, 1 equiv) and DIEA (2.90 g, 22.416 mmol, 1.5 equiv) in DCM (50 mL) was added Boc₂O (3.91 g, 17.933 mmol, 1.2 equiv) in portions at room temperature. The resulting mixture was stirred overnight at room temperature and concentrated under vacuum. The residue was purified by silica gel column chromatography, eluted with PE/EA (2:1) to afford benzyl (3R)-3-[(tert-butoxycarbonyl)(cyclobutyl)amino]pyrrolidine-1-carboxylate (4.5 g, 80%) as a solid.

LCMS (ES, m/z):375 [M+H]⁺.

### Synthesis of Intermediate B134

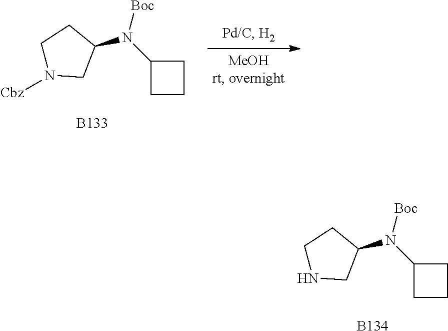

To a solution of benzyl (3R)-3-[(tert-butoxycarbonyl)(cyclobutyl)amino]pyrrolidine-1-carboxylate (1 g, 2.670 mmol, 1 equiv) in 50 mL MeOH was added Pd/C (10%, 0.2 g) under nitrogen atmosphere in a 250 mL round-bottom flask. The mixture was hydrogenated at room temperature for overnight under hydrogen atmosphere using a hydrogen balloon, filtered through a Celite pad and concentrated under reduced pressure to afford tert-butyl N-cyclobutyl-N-[(3R)-pyrrolidin-3-yl]carbamate (570 mg, 89%) as an oil. The crude product was used in the next step directly without further purification. LCMS (ES, m/z): 241 [M+H]⁺.

### Synthesis of Intermediate B135

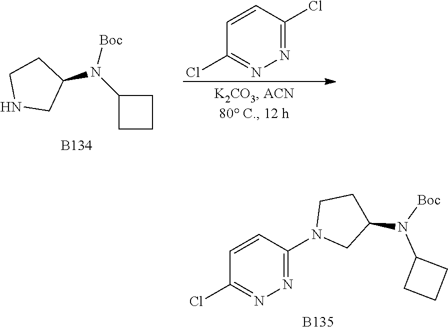

To a stirred mixture of tert-butyl N-cyclobutyl-N-[(3R)-pyrrolidin-3-yl]carbamate (570 mg, 2.372 mmol, 1 equiv) and 3,6-dichloropyridazine (423.95 mg, 2.846 mmol, 1.2 equiv) in ACN (5 mL) was added K₂CO₃ (983.29 mg, 7.116 mmol, 3 equiv) at room temperature. The resulting mixture was stirred for 12 hr at 80° C. The resulting mixture was concentrated under reduced pressure. The residue was purified by silica gel column chromatography, eluted with PE/EA (1:1) to afford tert-butyl N-[(3R)-1-(6-chloropyridazin-3-yl)pyrrolidin-3-yl]-N-cyclobutylcarbamate (670 mg, 80%) as a solid. LCMS (ES, m/z): 353 [M+H]⁺.

### Synthesis of Intermediate B136

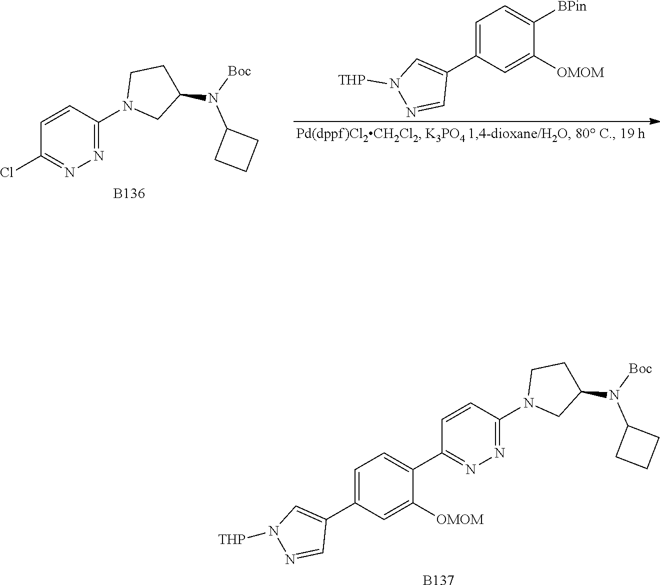

To a solution of tert-butyl N-[(3R)-1-(6-chloropyridazin-3-yl)pyrrolidin-3-yl]-N-cyclobutylcarbamate (200 mg, 0.567 mmol, 1 equiv) and 4-[3-(methoxymethoxy)-4-(4,4,5,5-tetramethyl-1,3,2-dioxaborolan-2-yl)phenyl]-1-(oxan-2-yl)pyrazole (281.80 mg, 0.680 mmol, 1.2 equiv) in 1,4-dioxane (2 mL) and H₂O (0.4 mL) was added K₃PO₄ (360.93 mg, 1.701 mmol, 3 equiv) and Pd(dppf)Cl₂—CH₂Cl₂ (46.17 mg, 0.057 mmol, 0.1 equiv) at room temperature under nitrogen atmosphere. After stirring for 19 hr at 80° C. under nitrogen atmosphere, the resulting mixture was concentrated under reduced pressure. The residue was purified by silica gel column chromatography, eluted with CH₂Cl₂/MeOH (50:1) to afford tert-butyl N-cyclobutyl-N-[(3R)-1-{6-[2-(methoxymethoxy)-4-[1-(oxan-2-yl)pyrazol-4-yl]phenyl]pyridazin-3-yl}pyrrolidin-3-yl]carbamate (190 mg, 55%) as an oil. LCMS (ES, m/z): 605 [M+H]⁺.

### Synthesis of Compound 114

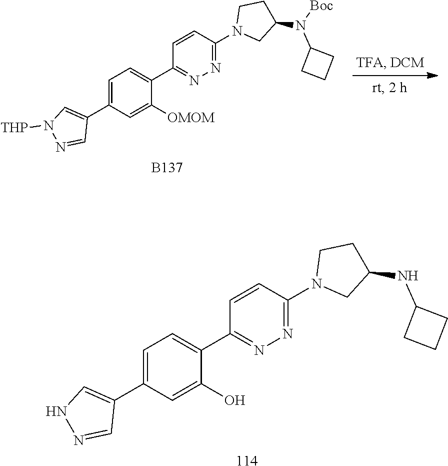

To a stirred solution of tert-butyl N-cyclobutyl-N-[(3R)-1-{6-[2-(methoxymethoxy)-4-[1-(oxan-2-yl)pyrazol-4-yl]phenyl]pyridazin-3-yl}pyrrolidin-3-yl]carbamate (190 mg, 0.314 mmol, 1 equiv) in DCM (2 mL) was added TFA (2 mL, 26.926 mmol, 85.70 equiv) at room temperature. The resulting mixture was stirred for 1 hr at room temperature and concentrated under reduced pressure. The crude product was purified by Prep-HPLC (Condition 5, Gradient 3). The crude product was further purified by chiral SFC with the following conditions (Column: CHIRAL ART Cellulose-SB, 3*25 cm, 5 m; Mobile Phase A: CO₂, Mobile Phase B: MEOH: DCM=2: 1(0.1% 2M NH₃-MeOH); Flow rate: 80 mL/min; Gradient: isocratic 35% B; Column Temperature(° C.): 35; Back Pressure(bar): 100; Wave Length: 220 nm; RT1(min): 13.5; RT2(min): 14.5; Sample Solvent: MeOH:DCM=1:1; Injection Volume: 1 mL; Number Of Runs: 40) to afford 2-{6-[(3R)-3-(cyclobutylamino)pyrrolidin-1-yl]pyridazin-3-yl}-5-(1H-pyrazol-4-yl)phenol (53.5 mg, 45.23%) as yellow solid. LCMS (ES, m/z): 377[M+H]^{+ 1}H NMR (300 MHz, DMSO-d6) δ 13.92 (s, 1H), 12.97 (s, 1H), 8.21 (d, J=9.9 Hz, 1H), 7.98 (s, 1H), 7.83 (d, J=8.2 Hz, 1H), 7.23-7.09 (m, 3H), 3.62 (dq, J=14.1, 6.4 Hz, 2H), 3.54-3.44 (m, 1H), 3.47-3.34 (m, 1H), 3.24 (q, J=7.5, 7.1 Hz, 2H), 2.09 (td, J=12.2, 6.2 Hz, 4H), 1.90-1.48 (m, 5H).

### Example 48: Synthesis of Compound 115

### Synthesis of Intermediate B138

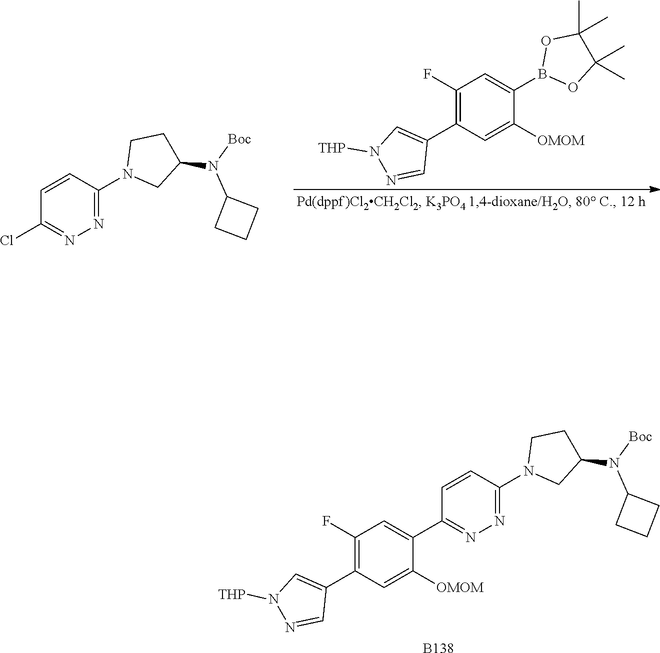

To a solution of tert-butyl N-[(3R)-1-(6-chloropyridazin-3-yl)pyrrolidin-3-yl]-N-cyclobutylcarbamate (160 mg, 0.453 mmol, 1 equiv) and 4-[2-fluoro-5-(methoxymethoxy)-4-(4,4,5,5-tetramethyl-1,3,2-dioxaborolan-2-yl)phenyl]-1-(oxan-2-yl)pyrazole (235.23 mg, 0.544 mmol, 1.2 equiv) in 1,4-dioxane (1.5 mL) and H₂O (0.3 mL) was added K₃PO₄ (288.75 mg, 1.359 mmol, 3 equiv) and Pd(dppf)Cl₂—CH₂Cl₂ (36.94 mg, 0.045 mmol, 0.1 equiv) at room temperature under nitrogen atmosphere. After stirring for 12 hr at 80° C. under nitrogen atmosphere, the resulting mixture was concentrated under reduced pressure. The residue was purified by silica gel column chromatography, eluted with CH₂Cl₂/MeOH (50:1) to afford ttert-butyl N-cyclobutyl-N-[(3R)-1-{6-[5-fluoro-2-(methoxymethoxy)-4-[1-(oxan-2-yl)pyrazol-4-yl]phenyl]pyridazin-3-yl}pyrrolidin-3-yl]carbamate (180 mg, 64%) as an oil. LCMS (ES, m/z): 623 [M+H]⁺.

### Synthesis of Compound 115

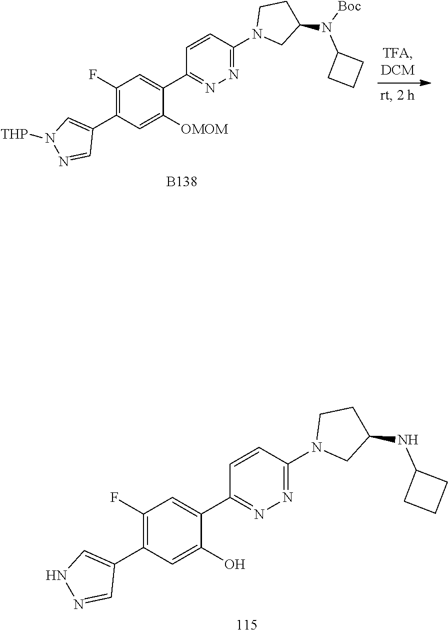

To a stirred solution of tert-butyl N-cyclobutyl-N-[(3R)-1-{6-[5-fluoro-2-(methoxymethoxy)-4-[1-(oxan-2-yl)pyrazol-4-yl]phenyl]pyridazin-3-yl}pyrrolidin-3-yl]carbamate (180 mg, 0.289 mmol, 1 equiv) in DCM (2 mL) was added TFA (2 mL, 26.926 mmol, 85.70 equiv) at room temperature. The resulting mixture was stirred for 1 h at room temperature and concentrated under reduced pressure. The crude product was purified by reverse phase flash (Condition 5, Gradient 2) to afford 2-{6-[(3R)-3-(cyclobutylamino)pyrrolidin-1-yl]pyridazin-3-yl}-4-fluoro-5-(1H-pyrazol-4-yl)phenol (11.9 mg, 10%) as a solid. LCMS (ES, m/z): 395[M+H]^{+ 1}H NMR (300 MHz, DMSO-d₆) δ 13.65 (s, 1H), 13.13 (s, 1H), 8.23 (d, J=9.8 Hz, 1H), 7.99 (s, 1H), 7.81 (d, J=12.7 Hz, 1H), 7.29 (d, J=7.0 Hz, 1H), 7.15 (d, J=9.8 Hz, 1H), 3.62 (s, 2H), 3.50 (t, J=7.8 Hz, 1H), 3.39 (t, J=5.6 Hz, 1H), 3.31-3.20 (m, 2H), 2.11 (dt, J=12.3, 6.3 Hz, 4H), 1.82 (dd, J=12.2, 6.5 Hz, 1H), 1.73-1.56 (m, 4H).

### Example 49: Synthesis of Compound 116

### Synthesis of Intermediate B139

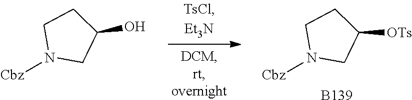 [SMILES: O=C(OCc1ccccc1)N1CC[C@@H](O)C1 ; "B139" ; Cc1ccc(S(=O)(=O)O[C@@H]2CCN(C(=O)OCc3ccccc3)C2)cc1]

To a stirred solution of benzyl (3R)-3-hydroxypyrrolidine-1-carboxylate (10 g, 45.197 mmol, 1 equiv) and Et₃N (9.15 g, 90.394 mmol, 2 equiv) in DCM (100 mL) was added TsCl (17.23 g, 90.394 mmol, 2 equiv) in portions at room temperature. The resulting mixture was stirred overnight at room temperature. The reaction solution was washed with 2×50 mL of brine and dried over anhydrous Na₂SO₄. After filtration, the filtrate was concentrated under reduced pressure to afford benzyl (3R)-3-[(4-methylbenzenesulfonyl)oxy]pyrrolidine-1-carboxylate (7.9 g, 46.56%) as an oil. LCMS (ES, m/z): 376 [M+H]⁺.

### Synthesis of Intermediate B140

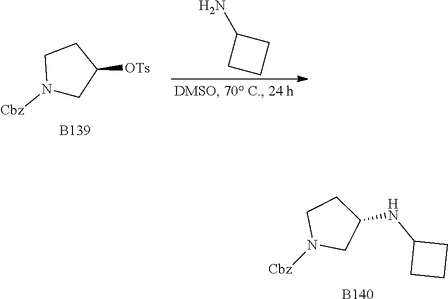

A solution of benzyl (3R)-3-[(4-methylbenzenesulfonyl)oxy]pyrrolidine-1-carboxylate (7.9 g, 21.042 mmol, 1 equiv) in DMSO (40 mL) was added cyclobutylamine (7.48 g, 105.210 mmol, 5 equiv) at room temperature. The resulting mixture was stirred for 24 hr at 70° C. The resulting mixture was diluted with H₂O (150 mL) and extracted with EtOAc (3×100 mL). The combined organic layers were washed with brine (3×100 mL), dried over anhydrous Na₂SO₄. After filtration, the filtrate was concentrated under reduced pressure to afford benzyl (3S)-3-(cyclobutylamino)pyrrolidine-1-carboxylate (3.8 g, 66%) as a solid. LCMS (ES, m/z): 275 [M+H]⁺.

### Synthesis of Intermediate B141

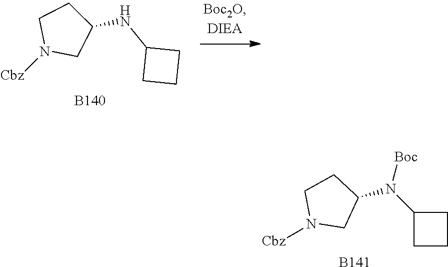 [SMILES: "B140" ; O=C(OCc1ccccc1)N1CC[C@H](NC2CCC2)C1 ; "B141" ; CC(C)(C)OC(=O)N(C1CCC1)[C@H]1CCN(C(=O)OCc2ccccc2)C1]

To a stirred solution of benzyl (3S)-3-(cyclobutylamino)pyrrolidine-1-carboxylate (3.8 g, 13.850 mmol, 1 equiv) and DIEA (2.69 g, 20.775 mmol, 1.5 equiv) in DCM (50 mL) was added Boc₂O (3.63 g, 16.620 mmol, 1.2 equiv) in portions at room temperature. The resulting mixture was stirred overnight at room temperature and concentrated under vacuum. The residue was purified by silica gel column chromatography, eluted with PE/EA (2:1) to afford benzyl (3S)-3-[(tert-butoxycarbonyl)(cyclobutyl)amino]pyrrolidine-1-carboxylate (4 g, 77%) as a solid. LCMS (ES, m/z):375 [M+H]⁺.

### Synthesis of Intermediate B142

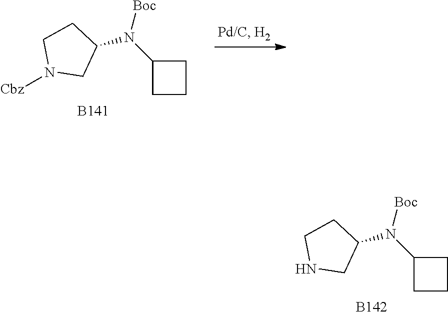

To a solution of benzyl (3S)-3-[(tert-butoxycarbonyl)(cyclobutyl)amino]pyrrolidine-1-carboxylate (1 g, 2.670 mmol, 1 equiv) in 50 mL MeOH was added Pd/C (10%, 0.2 g) under nitrogen atmosphere in a 250 mL round-bottom flask. The mixture was hydrogenated at room temperature for 2 h under hydrogen atmosphere using a hydrogen balloon, filtered through a Celite pad and concentrated under reduced pressure to afford tert-butyl N-cyclobutyl-N-[(3S)-pyrrolidin-3-yl]carbamate (560 mg, 87%) as an oil. The crude product was used in the next step directly without further purification. LCMS (ES, m/z): 241 [M+H]⁺.

### Synthesis of Intermediate B143

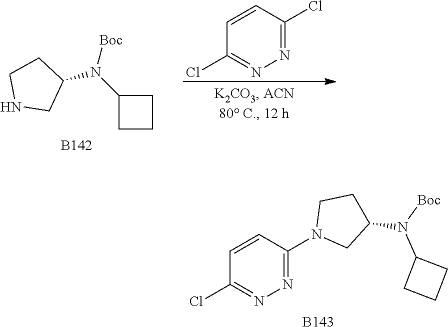

To a stirred mixture of tert-butyl N-cyclobutyl-N-[(3S)-pyrrolidin-3-yl]carbamate (560 mg, 2.330 mmol, 1 equiv) and 3,6-dichloropyridazine (347.09 mg, 2.330 mmol, 1 equiv) in ACN (5 mL) was added K₂CO₃ (966.04 mg, 6.990 mmol, 3 equiv) at room temperature. The resulting mixture was stirred for 12 h at 80° C. The resulting mixture was concentrated under reduced pressure. The residue was purified by silica gel column chromatography, eluted with PE/EA (1:1) to afford tert-butyl N-[(3S)-1-(6-chloropyridazin-3-yl)pyrrolidin-3-yl]-N-cyclobutylcarbamate (700 mg, 85%) as a solid. LCMS (ES, m/z): 353 [M+H]⁺.

### Synthesis of Intermediate B144

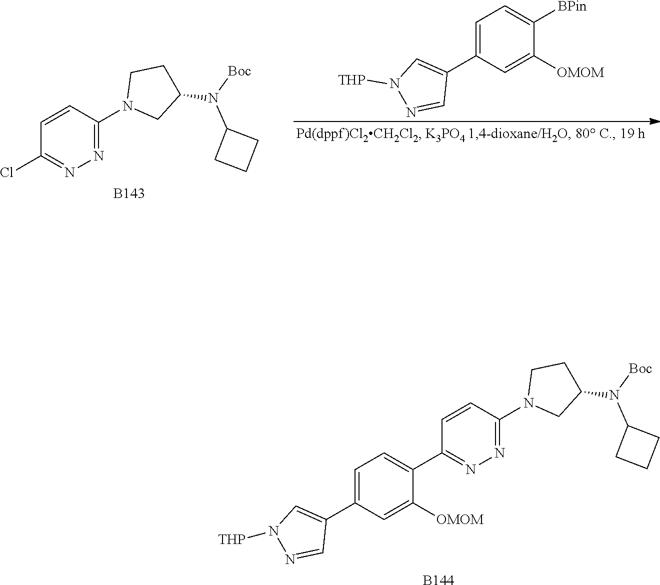

To a solution of tert-butyl N-[(3S)-1-(6-chloropyridazin-3-yl)pyrrolidin-3-yl]-N-cyclobutylcarbamate (210 mg, 0.595 mmol, 1 equiv) and 4-[3-(methoxymethoxy)-4-(4,4,5,5-tetramethyl-1,3,2-dioxaborolan-2-yl)phenyl]-1-(oxan-2-yl)pyrazole (295.89 mg, 0.714 mmol, 1.2 equiv) in 1,4-dioxane (2 mL) and H₂O (0.4 mL) was added K₃PO₄ (378.98 mg, 1.785 mmol, 3 equiv) and Pd(dppf)Cl₂—CH₂Cl₂ (48.48 mg, 0.059 mmol, 0.1 equiv) at room temperature under nitrogen atmosphere. After stirring for 19 hr at 80° C. under nitrogen atmosphere, the resulting mixture was concentrated under reduced pressure. The residue was purified by silica gel column chromatography, eluted with CH₂Cl₂/MeOH (50:1) to afford tert-butyl N-cyclobutyl-N-[(3S)-1-{6-[2-(methoxymethoxy)-4-[1-(oxan-2-yl)pyrazol-4-yl]phenyl]pyridazin-3-yl}pyrrolidin-3-yl]carbamate (210 mg, 58%) as an oil. LCMS (ES, m/z): 605 [M+H]⁺.

### Synthesis of Compound 116

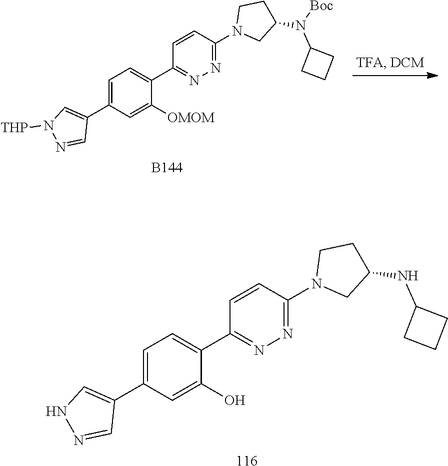

To a stirred solution of tert-butyl N-cyclobutyl-N-[(3S)-1-{6-[2-(methoxymethoxy)-4-[1-(oxan-2-yl)pyrazol-4-yl]phenyl]pyridazin-3-yl}pyrrolidin-3-yl]carbamate (210 mg, 0.347 mmol, 1 equiv) in DCM (2 mL) was added TFA (2 mL, 26.926 mmol, 85.70 equiv) at room temperature. The resulting mixture was stirred for 2 h at room temperature and concentrated under reduced pressure. The crude product was purified by Prep-HPLC(Condition 5, Gradient 3).

The crude product was further purified by chiral SFC with the following conditions (Column: CHIRAL ART Cellulose-SB, 3*25 cm, 5 m; Mobile Phase A: CO₂, Mobile Phase B: MEOH: DCM=2: 1(0.1% 2M NH₃-MeOH); Flow rate: 80 mL/min; Gradient: isocratic 35% B; Column Temperature(° C.): 35; Back Pressure(bar): 100; Wave Length: 220 nm; RT1(min): 13.5; RT2(min): 14.5; Sample Solvent: MeOH:DCM=1:1; Injection Volume: 2 mL; Number Of Runs: 35) to afford 2-{6-[(3S)-3-(cyclobutylamino)pyrrolidin-1-yl]pyridazin-3-yl}-5-(1H-pyrazol-4-yl)phenol (54.7 mg, 42%) as a solid. LCMS (ES, m/z): 377[M+H]^{+ 1}H NMR (300 MHz, DMSO-d₆) δ 13.92 (s, 1H), 12.97 (s, 1H), 8.22 (s, 1H), 8.20 (d, J=9.8 Hz, 1H), 7.99 (s, 1H), 7.83 (d, J=8.2 Hz, 1H), 7.23-7.13 (m, 2H), 7.14 (d, J=9.8 Hz, 1H), 3.70-3.34 (m, 3H), 3.32-3.18 (m, 2H), 2.12 (td, J=12.6, 11.3, 5.3 Hz, 3H), 1.80 (ddd, J=29.3, 14.7, 7.1 Hz, 1H), 1.74-1.48 (m, 4H).

### Example 50: Synthesis of Compound 120

### Synthesis of Intermediate B145

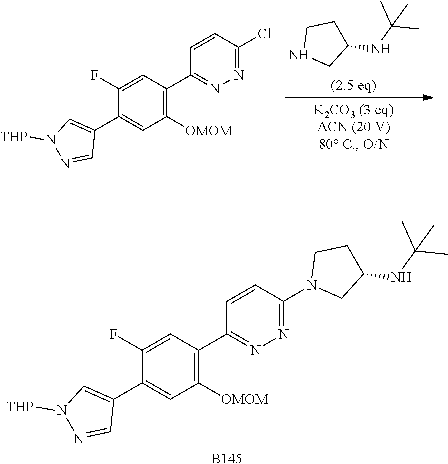

A solution of 3-chloro-6-[5-fluoro-2-(methoxymethoxy)-4-[1-(oxan-2-yl)pyrazol-4-yl]phenyl]pyridazine (60 mg, 0.143 mmol, 1 equiv), (3S)—N-tert-butylpyrrolidin-3-amine (50.94 mg, 0.357 mmol, 2.5 equiv) and K₂CO₃ (59.39 mg, 0.429 mmol, 3 equiv) in acetonitrile was stirred for 2 days at 80° C. under nitrogen atmosphere. The resulting mixture was extracted with CH₂Cl₂ (5×10 mL). The combined organic layers were dried over anhydrous Na₂SO₄. The filtrate was concentrated under reduced pressure to afford the crude product (3S)—N-(tert-butyl)-1-(6-(5-fluoro-2-(methoxymethoxy)-4-(1-(tetrahydro-2H-pyran-2-yl)-1H-pyrazol-4-yl)phenyl)pyridazin-3-yl)pyrrolidin-3-amine as a solid.

### Synthesis of Intermediate B146

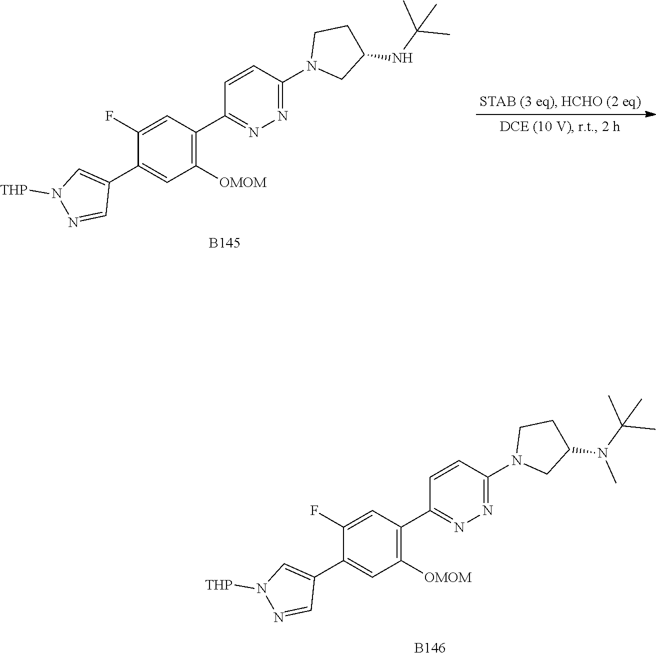

A solution of (3s)-N-tert-butyl-1-{6-[5-fluoro-2-(methoxymethoxy)-4-[1-(oxan-2-yl)pyrazol-4-yl]phenyl]pyridazin-3-yl}pyrrolidin-3-amine (10 mg, 0.019 mmol, 1 equiv) and HCHO (1.14 mg, 0.038 mmol, 2 equiv) in DCE (1 mL) was stirred for 10 min at room temperature. To the above mixture was added STAB (12.12 mg, 0.057 mmol, 3 equiv) in portions at room temperature. The resulting mixture was extracted with CH₂Cl₂ (3×20 mL). The combined organic layers were washed with saturated aqueous NaCl (50 mL), dried over anhydrous Na₂SO₄. The filtrate was concentrated under reduced pressure to afford the crude product (3S)—N-tert-butyl-1-{6-[5-fluoro-2-(methoxymethoxy)-4-[1-(oxan-2-yl)pyrazol-4-yl]phenyl]pyridazin-3-yl}-N-methylpyrrolidin-3-amine as a solid.

### Synthesis of Compound 120

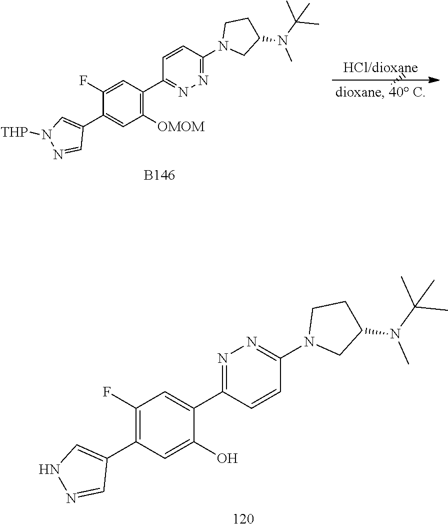

Into a 100 mL round-bottom flask were added (3S)—N-tert-butyl-1-{6-[5-fluoro-2-(methoxymethoxy)-4-[1-(oxan-2-yl)pyrazol-4-yl]phenyl]pyridazin-3-yl}-N-methylpyrrolidin-3-amine (60 mg, 0.186 mmol, 1 equiv) and HCl(gas) in 1,4-dioxane (4M/L; 40 mL) at 40° C. The resulting mixture was stirred for 6 h at room temperature. The mixture was neutralized to pH 7 with saturated NaHCO₃ (aq.). The crude product was purified by Chiral-Prep-HPLC (Condition 7, Gradient 1) to afford 2-{6-[(3S)-3-[tert-butyl(methyl)amino]pyrrolidin-1-yl]pyridazin-3-yl}-4-fluoro-5-(1H-pyrazol-4-yl)phenol (8.6 mg, 8%) as a solid. LCMS (ES, m/z): 411 [M+H]^{+ 1}H NMR (400 MHz, DMSO-d₆) δ 13.62 (s, 1H), 13.10 (s, 1H), 8.22 (d, J=9.8 Hz, 2H), 7.98 (s, 1H), 7.81 (d, J=12.7 Hz, 1H), 7.28 (d, J=7.0 Hz, 1H), 7.18 (d, J=9.7 Hz, 1H), 4.00-3.91 (m, 1H), 3.70 (t, J=9.7 Hz, 1H), 3.49 (t, J=9.4 Hz, 1H), 3.44-3.35 (m, 1H), 3.27 (s, 1H), 2.22 (s, 3H), 2.05 (t, J=10.4, 9.8 Hz, 1H), 1.93 (s, 1H), 1.10 (s, 9H).

### Example 51: Synthesis of Compound 122, 123, and 171

### Synthesis of Intermediate B147

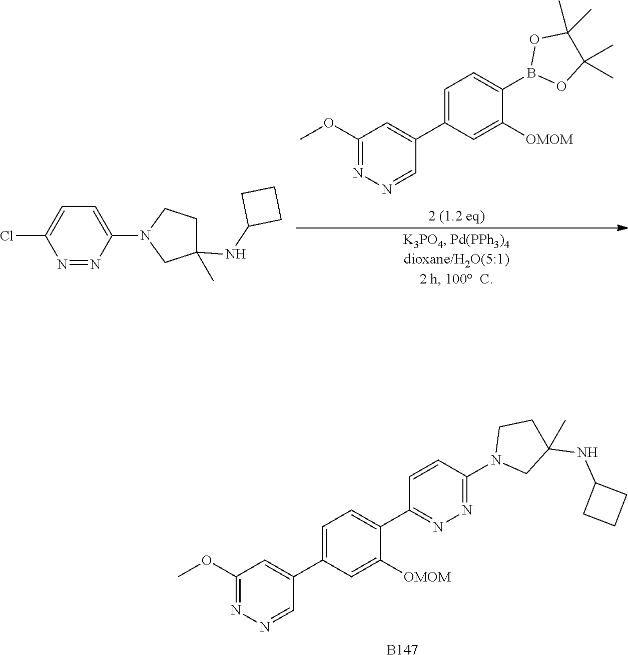

To a solution of 1-(6-chloropyridazin-3-yl)-N-cyclobutyl-3-methylpyrrolidin-3-amine (200 mg, 0.750 mmol, 1.0 equiv) and 3-methoxy-5-[3-(methoxymethoxy)-4-(4,4,5,5-tetramethyl-1,3,2-dioxaborolan-2-yl) phenyl]pyridazine (334.88 mg, 0.900 mmol, 1.2 equiv) in 1,4-dioxane (2 mL) and H₂O (400 uL) were added K₃PO₄ (397.84 mg, 1.875 mmol, 2.5 equiv) and Pd(PPh₃)4 (86.64 mg, 0.075 mmol, 0.1 equiv). After stirring for 2 h at 100° C. under a nitrogen atmosphere. The resulting mixture was extracted with EtOAc (30×mL). The combined organic layers were washed with brine (3×30 mL), dried over anhydrous Na₂SO₄. After filtration, the filtrate was concentrated under reduced pressure. The residue was purified by Prep-TLC (PE/EA 1:4) to afford N-cyclobutyl-1-{6-[2-(methoxymethoxy)-4-(6-methoxypyridazin-4-yl)phenyl]pyridazin-3-yl}-3-methylpyrrolidin-3-amine (200 mg, 56%) as a solid. LCMS:(ES, m/z):

### Synthesis of Compound 171

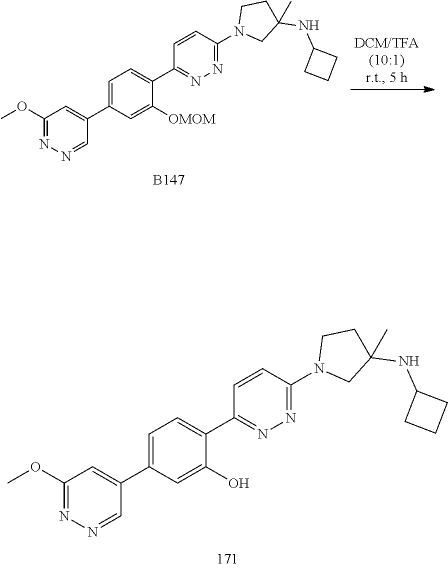

A solution of N-cyclobutyl-1-{6-[2-(methoxymethoxy)-4-(6-methoxypyridazin-4-yl) phenyl]pyridazin-3-yl}-3-methylpyrrolidin-3-amine (200 mg, 0.420 mmol, 1 equiv) in DCM (2 mL) was treated with TFA (1 mL) for 5 h at room temperature. The mixture basified to pH 9 with saturated Na₂CO₃ (aq.). The resulting mixture was extracted with CH₂Cl₂ (3×20 mL). The combined organic layers were washed with water (3×20 mL), dried over anhydrous Na₂SO₄. After filtration, the filtrate was concentrated under reduced pressure. The crude product was purified by Chiral-Prep-HPLC (condition 10, Gradient 1) to afford 2-{6-[3-(cyclobutylamino)-3-methylpyrrolidin-1-yl]pyridazin-3-yl}-5-(6-methoxypyridazin-4-yl) phenol (160 mg, 88%) as a solid. LCMS: (ES, m/z): 433 [M+H]⁺¹H NMR: (400 MHz, DMSO-d₆) δ14.11 (s, 1H), 9.36 (d, J=1.9 Hz, 1H), 8.31 (d, J=9.8 Hz, 1H), 8.05 (d, J=8.3 Hz, 1H), 7.56 (d, J=1.9 Hz, 1H), 7.52-7.43 (m, 2H), 7.17 (d, J=9.7 Hz, 1H), 4.08 (s, 3H), 3.63-3.35 (q, 5H), 2.15 (ddd, J=11.2, 7.4, 3.7 Hz, 2H), 2.02 (t, J=10.3 Hz, 1H), 1.80 (d, J=40.4 Hz, 3H), 1.56 (d, J=9.9 Hz, 2H), 1.23 (s, 3H)

### Synthesis of Compounds 122 and 123

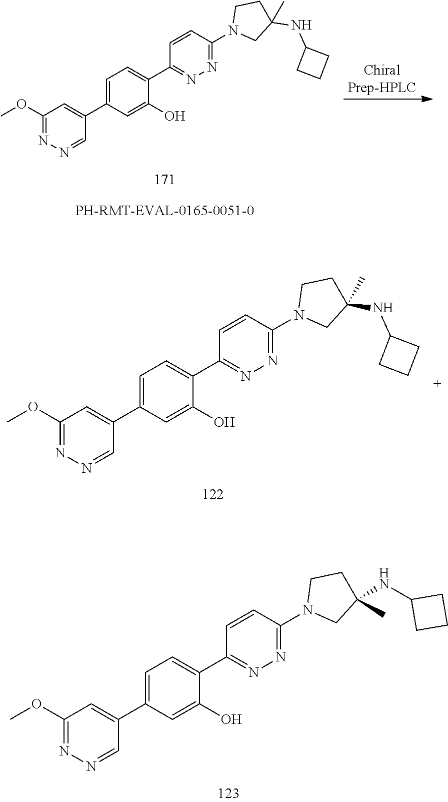 [SMILES: COc1cc(-c2ccc(-c3ccc(N4CCC(C)(NC5CCC5)C4)nn3)c(O)c2)cnn1 ; COc1cc(-c2ccc(-c3ccc(N4CC[C@@](C)(NC5CCC5)C4)nn3)c(O)c2)cnn1 ; COc1cc(-c2ccc(-c3ccc(N4CC[C@](C)(NC5CCC5)C4)nn3)c(O)c2)cnn1]

2-{6-[3-(cyclobutylamino)-3-methylpyrrolidin-1-yl]pyridazin-3-yl}-5-(6-methoxypyridazin-4-yl) phenol (120 mg) was purified with the following conditions Column: CHIRAL ART Cellulose-SB, 4.6×100 mm, 3.Oum; Mobile Phase A: MTBE(0.1% DEA): MeOH=50: 50; Flow rate: 1 mL/min; Gradient: 0% B to 0% B; Injection Volume: Sul mL. This resulted in 2-{6-[(3R)-3-(cyclobutylamino)-3-methylpyrrolidin-1-yl]pyridazin-3-yl}-5-(6-methoxypyridazin-4-yl) phenol (41.6 mg, 34.11%) as a white solid and 2-{6-[(3R)-3-(cyclobutylamino)-3-methylpyrrolidin-1-yl]pyridazin-3-yl}-5-(6-methoxypyridazin-4-yl)phenol (41.6 mg, 34.11%) as a solid. Compound 122: LCMS: (ES, m/z): 433 [M+H]⁺¹H NMR: (400 MHz, DMSO-d₆) δ14.11 (s, 1H), 9.36 (d, J=1.9 Hz, 1H), 8.31 (d, J=9.8 Hz, 1H), 8.05 (d, J=8.3 Hz, 1H), 7.56 (d, J=1.9 Hz, 1H), 7.52-7.43 (m, 2H), 7.17 (d, J=9.7 Hz, 1H), 4.08 (s, 3H), 3.78-3.65 (s, 1H), 3.63 (s, 1H), 3.55 (s, 1H), 3.46 (s, 1H)3.30-3.19 (s, 1H), 2.15 (ddd, J=11.2, 7.4, 3.7 Hz, 2H), 2.02 (t, J=10.3 Hz, 1H), 1.80 (d, J=40.4 Hz, 3H), 1.56 (d, J=9.9 Hz, 2H), 1.23 (s, 3H) Compound 123: LCMS: (ES, m/z): 433 [M+H]⁺¹H NMR: (400 MHz, DMSO-d₆) δ614.11 (s, 1H), 9.36 (d, J=1.9 Hz, 1H), 8.31 (d, J=9.8 Hz, 1H), 8.05 (d, J=8.3 Hz, 1H), 7.56 (d, J=1.9 Hz, 1H), 7.52-7.43 (m, 2H), 7.17 (d, J=9.7 Hz, 1H), 4.08 (s, 3H), 3.78-3.65 (s, 1H), 3.63 (s, 1H), 3.55 (s, 1H), 3.46 (s, 1H)3.30-3.19 (s, 1H), 2.15 (ddd, J=11.2, 7.4, 3.7 Hz, 2H), 2.02 (t, J 10.3 Hz, 1H), 1.80 (d, J=40.4 Hz, 3H), 1.56 (d, J=9.9 Hz, 2H), 1.23 (s, 3H)

### Example 52: Synthesis of Compounds 125 and 126

### Synthesis of Intermediate B148

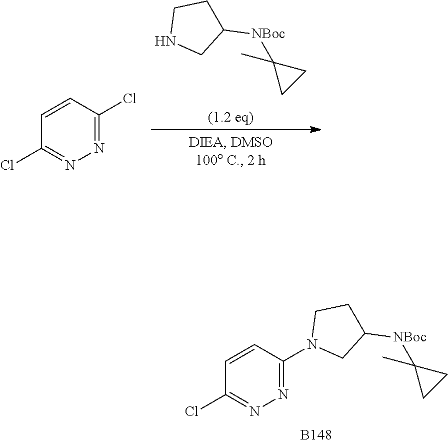

A solution of pyridazine, 3,6-dichloro- (1 g, 6.713 mmol, 1 equiv) and tert-butyl N-(1-methylcyclopropyl)-N-(pyrrolidin-3-yl)carbamate (1.94 g, 8.056 mmol, 1.2 equiv), DIEA (2.60 g, 20.139 mmol, 3 equiv) in DMSO (10 mL) was stirred for 2 h at 100° C. The mixture was allowed to cool down to room temperature. The resulting mixture was poured into water. The resulting mixture was filtered and the filter cake was washed with water (1×5 mL). The cake was concentrated under reduced pressure. This resulted in tert-butyl N-[1-(6-chloropyridazin-3-yl)pyrrolidin-3-yl]-N-(1-methylcyclopropyl)carbamate (2 g, 84%) as a solid. LCMS:(ES, m/z):353

### Synthesis of Intermediate B149

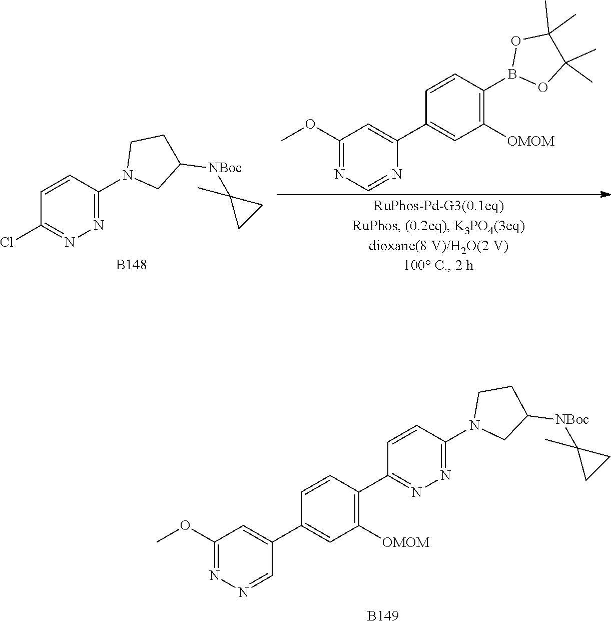

A solution of tert-butyl N-[1-(6-chloropyridazin-3-yl)pyrrolidin-3-yl]-N-(1-methylcyclopropyl)carbamate (200 mg, 0.567 mmol, 1 equiv) and 3-methoxy-5-[3-(methoxymethoxy)-4-(4,4,5,5-tetramethyl-1,3,2-dioxaborolan-2-yl)phenyl]pyridazine (422 mg, 1.134 mmol, 2 equiv), RuPhos Palladacycle Gen.3 (47 mg, 0.057 mmol, 0.1 equiv), RuPhos (53 mg, 0.113 mmol, 0.2 equiv),K₃PO₄ (361 mg, 1.701 mmol, 3 equiv) in 1,4-dioxane (1.6 mL), water (0.4 mL) was stirred for 2 h at 100° C. under nitrogen atmosphere. The mixture was allowed to cool down to room temperature. The resulting mixture was poured into water. The resulting mixture was extracted with EA (1×9 mL) dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-(1-{6-[2-(methoxymethoxy)-4-(6-methoxypyridazin-4-yl)phenyl]pyridazin-3-yl}pyrrolidin-3-yl)-N-(1-methylcyclopropyl)carbamate (300 mg, 94%) as a solid. LCMS:(ES, m/z):563

### Synthesis of Intermediates B5 and B151

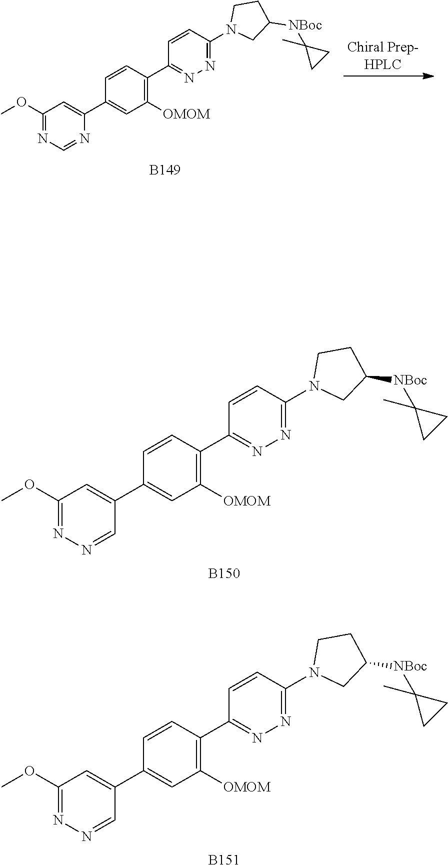

The crude product (150 mg) was purified by Prep-HPLC with the following conditions to afford B150 (59 mg) as a solid and B151 (61 mg) as a solid.

### Synthesis of Compound 125

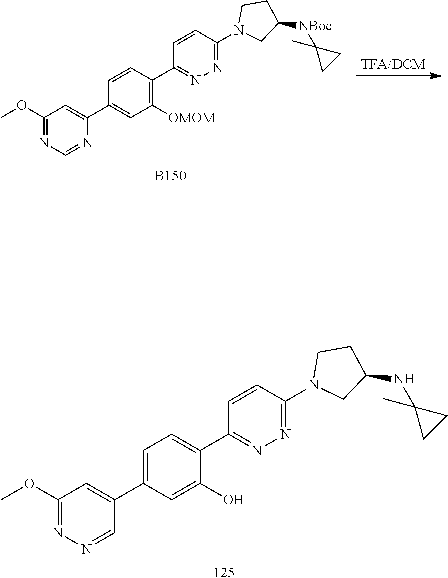

A solution of tert-butyl N-[(3R)-1-{6-[2-(methoxymethoxy)-4-(6-methoxypyridazin-4-yl)phenyl]pyridazin-3-yl}pyrrolidin-3-yl]-N-(1-methylcyclopropyl)carbamate (59 mg, 0.105 mmol, 1 equiv) in DCM (1.8 mL). TFA (0.6 mL) was stirred for 1 h at room temperature. The resulting mixture was concentrated under reduced pressure. The crude product was purified by Prep-HPLC (Condition 10, Gradient 2) to afford 5-(6-methoxypyridazin-4-yl)-2-{6-[(3R)-3-[(1-methylcyclopropyl)amino]pyrrolidin-1-yl]pyridazin-3-yl}phenol (18.4 mg, 41%) as a solid. LCMS:(ES, m/z):419 ¹HNMR: (400 MHz, DMSO-d₆) δ 14.11 (s, 1H), 9.35 (d, J=1.9 Hz, 1H), 8.29 (d, J=9.9 Hz, 1H), 8.04 (d, J=8.3 Hz, 1H), 7.55 (d, J=2.0 Hz, 1H), 7.52-7.44 (m, 2H), 7.17 (d, J=9.7 Hz, 1H), 4.09 (s, 3H), 3.76-3.66 (m, 1H), 3.64-3.60 (q, J=6.0 Hz, 2H), 3.49 (q, J=8.7, 8.1 Hz, 1H), 3.29 (s, 1H), 2.40 (s, 1H), 2.13 (dq, J=12.5, 6.2 Hz, 1H), 1.89 (dt, J 12.2, 6.9 Hz, 1H), 1.26 (s, 3H), 0.53-0.40 (m, 2H), 0.39-0.27 (m, 2H).

### Synthesis of Compound 126

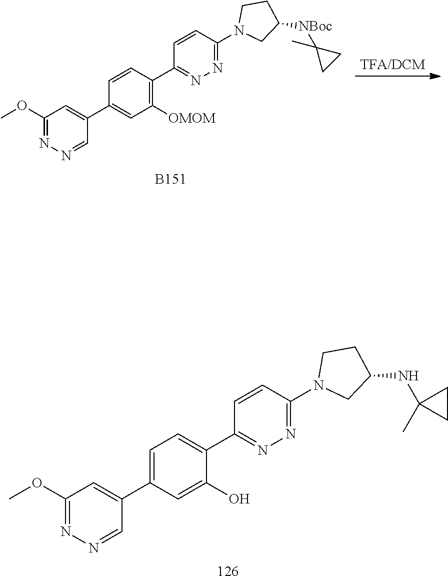

A solution of tert-butyl N-[(3S)-1-{6-[2-(methoxymethoxy)-4-(6-methoxypyridazin-4-yl)phenyl]pyridazin-3-yl}pyrrolidin-3-yl]-N-(1-methylcyclopropyl)carbamate (61 mg, 0.108 mmol, 1 equiv) in DCM (1.8 mL). TFA (0.6 mL) was stirred for 1 h at room temperature. The resulting mixture was concentrated under reduced pressure. The crude product was purified by Prep-(Condition 10, Gradient 2) to afford 5-(6-methoxypyridazin-4-yl)-2-{6-[(3S)-3-[(1-methylcyclopropyl)amino]pyrrolidin-1-yl]pyridazin-3-yl}phenol (19.2 mg, 42%) as a solid. LCMS:(ES, m/z):419 ¹HNMR: (400 MHz, DMSO-d₆) δ 14.11 (s, 1H), 9.35 (d, J=1.9 Hz, 1H), 8.29 (d, J=9.8 Hz, 1H), 8.04 (d, J=8.3 Hz, 1H), 7.55 (d, J=1.9 Hz, 1H), 7.52-7.44 (m, 2H), 7.17 (d, J=9.8 Hz, 1H), 4.09 (s, 3H), 3.72 (dd, J=10.4, 6.1 Hz, 1H), 3.65-3.60 (p, J=6.0 Hz, 2H), 3.50 (t, J=8.1 Hz, 1H), 3.29 (s, 1H), 2.40 (s, 1H), 2.13 (dq, J=12.3, 6.1 Hz, 1H), 1.88 (dq, J=13.8, 7.1 Hz, 1H), 1.26 (s, 3H), 0.52-0.40 (m, 2H), 0.33 (q, J=2.6, 2.0 Hz, 2H).

### Example 53: Synthesis of Compounds 137 and 140

### Synthesis of Intermediate B152

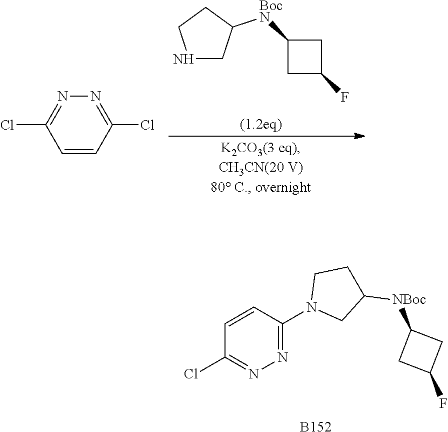

A solution of pyridazine, 3,6-dichloro- (500 mg, 3.356 mmol, 1 equiv), tert-butyl N-(pyrrolidin-3-yl)-N-[(1s,3s)-3-fluorocyclobutyl]carbamate (1040.49 mg, 4.027 mmol, 1.2 equiv) and K₂CO₃ (1391.61 mg, 10.068 mmol, 3 equiv) in CH₃CN (10 mL) was stirred for overnight at 80° C. under. The resulting mixture was filtered and the filter cake was washed with CH₂Cl₂ (3×30 mL). The filtrate was concentrated under reduced pressure. The residue was purified by silica gel column chromatography, eluted with CH₂Cl₂/MeOH (5:1) to afford tert-butyl N-[1-(6-chloropyridazin-3-yl)pyrrolidin-3-yl]-N-[(1s,3s)-3-fluorocyclobutyl]carbamate (1 g, 72.31%) as a solid. LCMS (ES, m/z): 371 [M+H]⁺

### Synthesis of Intermediate B153

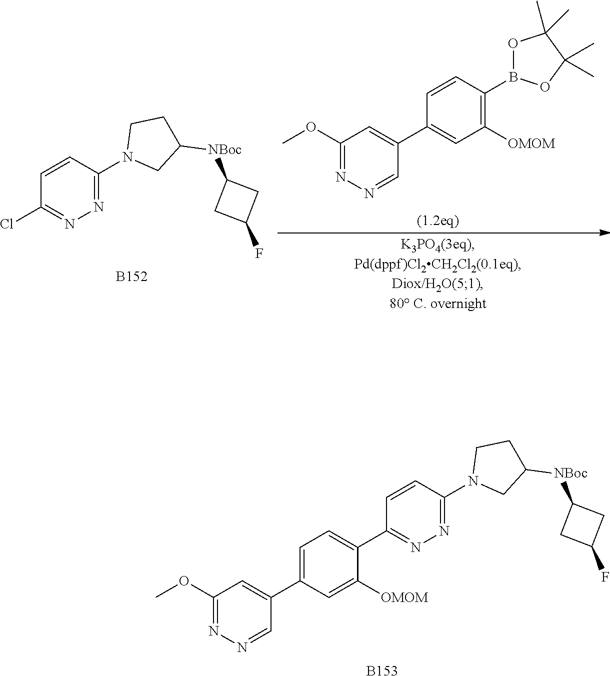

To a solution of tert-butyl N-[1-(6-chloropyridazin-3-yl)pyrrolidin-3-yl]-N-[(1s,3s)-3-fluorocyclobutyl]carbamate (500 mg, 1.348 mmol, 1 equiv) and 3-methoxy-5-[3-(methoxymethoxy)-4-(4,4,5,5-tetramethyl-1,3,2-dioxaborolan-2-yl)phenyl]pyridazine (602.23 mg, 1.618 mmol, 1.2 equiv) in 1,4-dioxane (10 mL) and H₂O (2 mL) were added Pd(dppf)Cl₂·CH₂Cl₂ (109.83 mg, 0.135 mmol, 0.1 equiv) and K₃PO₄ (858.56 mg, 4.044 mmol, 3 equiv). After stirring for overnight at 80° C. under a nitrogen atmosphere, the resulting mixture was concentrated under reduced pressure. The residue was purified by silica gel column chromatography, eluted with CH₂Cl₂/MeOH (10:1) to afford tert-butyl N-(1-{6-[2-(methoxymethoxy)-4-(6-methoxypyridazin-4-yl)phenyl]pyridazin-3-yl}pyrrolidin-3-yl)-N-[(1s,3s)-3-fluorocyclobutyl]carbamate (320 mg, 41%) as a solid. LCMS (ES, m/z): 581 [M+H]⁺

### Synthesis of Compounds 137 and 140

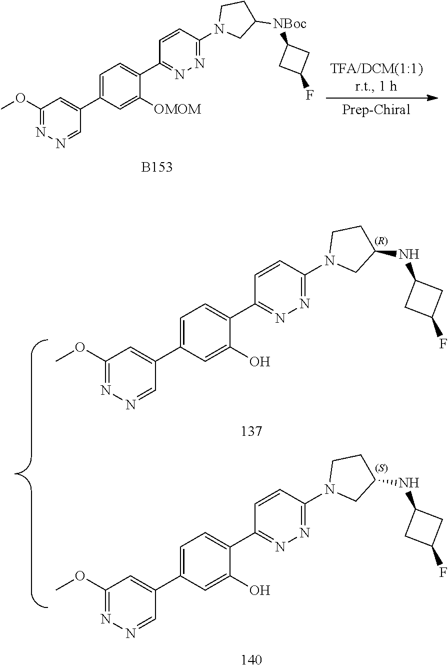

A solution of tert-butyl N-(1-{6-[2-(methoxymethoxy)-4-(6-methoxypyridazin-4-yl)phenyl]pyridazin-3-yl}pyrrolidin-3-yl)-N-[(1s,3s)-3-fluorocyclobutyl]carbamate (200 mg, 0.344 mmol, 1 equiv) and TFA (5 mL, 67.315 mmol, 195.44 equiv) in DCM (5 mL) was stirred for 1 h at room temperature. The resulting mixture was concentrated under reduced pressure. The crude product (100 mg) was purified by Prep-Chiral-HPLC (Condition 11, Gradient 1) to afford 5-(6-methoxypyridazin-4-yl)-2-{6-[(3S)-3-{[(1s,3s)-3-fluorocyclobutyl]amino}pyrrolidin-1-yl]pyridazin-3-yl}phenol (18.2 mg) as a yellow solid and 5-(6-methoxypyridazin-4-yl)-2-{6-[(3R)-3-{[(1s,3s)-3-fluorocyclobutyl]amino}pyrrolidin-1-yl]pyridazin-3-yl}phenol (25.6 mg) as a solid.

### Example 54: Synthesis of Compounds 138 and 139

### Synthesis of Intermediate B154

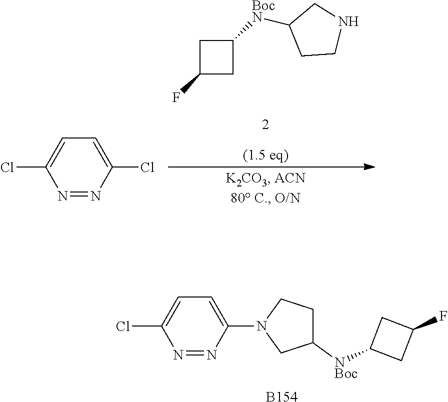

A solution of tert-butyl N-(pyrrolidin-3-yl)-N-[(1r,3r)-3-fluorocyclobutyl]carbamate (1300.62 mg, 5.034 mmol, 1.5 equiv) in ACN (20 mL, 380.484 mmol, 113.36 equiv) was treated with pyridazine, 3,6-dichloro- (500 mg, 3.356 mmol, 1 equiv) and K₂CO₃ (1391.61 mg, 10.068 mmol, 3 equiv) overnight at 80° C. The resulting mixture was filtered and the filter cake was washed with CH₂Cl₂ (3×5 mL). The filtrate was concentrated under reduced pressure. The residue was purified by silica gel column chromatography, eluted with PE/EA (3:2) to afford tert-butyl N-[1-(6-chloropyridazin-3-yl)pyrrolidin-3-yl]-N-[(1r,3r)-3-fluorocyclobutyl]carbamate (900 mg, 72%) as a solid.

### Synthesis of Intermediate B155

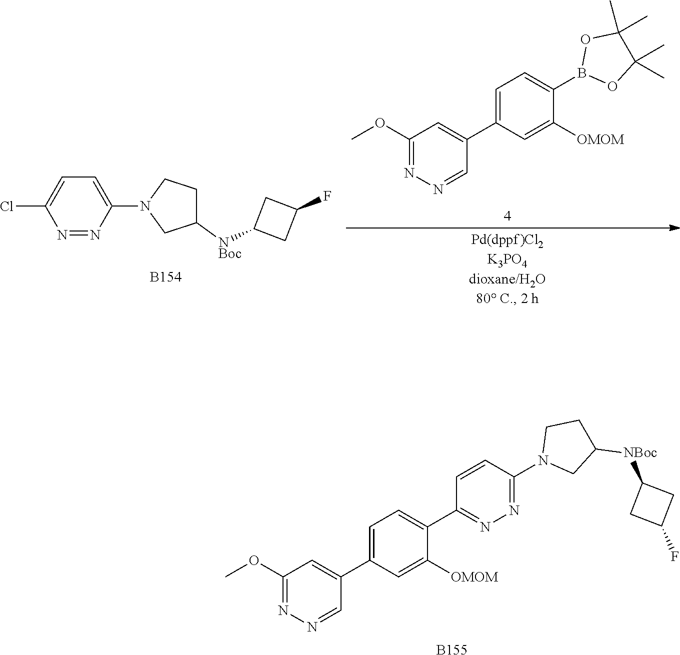

A solution of tert-butyl N-[1-(6-chloropyridazin-3-yl)pyrrolidin-3-yl]-N-[(1r,3r)-3-fluorocyclobutyl]carbamate (200 mg, 0.539 mmol, 1 equiv) in dioxane (5 mL)/H₂O (1 mL) was treated with 3-methoxy-5-[3-(methoxymethoxy)-4-(4,4,5,5-tetramethyl-1,3,2-dioxaborolan-2-yl)phenyl]pyridazine (401.49 mg, 1.078 mmol, 2 equiv), Pd(dppf)Cl₂·CH₂Cl₂ (39.46 mg, 0.054 mmol, 0.1 equiv) and K₃PO₄ (343.42 mg, 1.617 mmol, 3 equiv) for 2 h at 80° C. under nitrogen atmosphere. The reaction was quenched with water (5 mL) at 0° C. The aqueous layer was extracted with EtOAc (3×5 mL). The combined organic layers were dried over anhydrous Na₂SO₄. After filtration, the filtrate was concentrated under reduced pressure. The crude product(250 mg) was used in the next step directly without further purification.

### Synthesis of Compounds 138 and 139

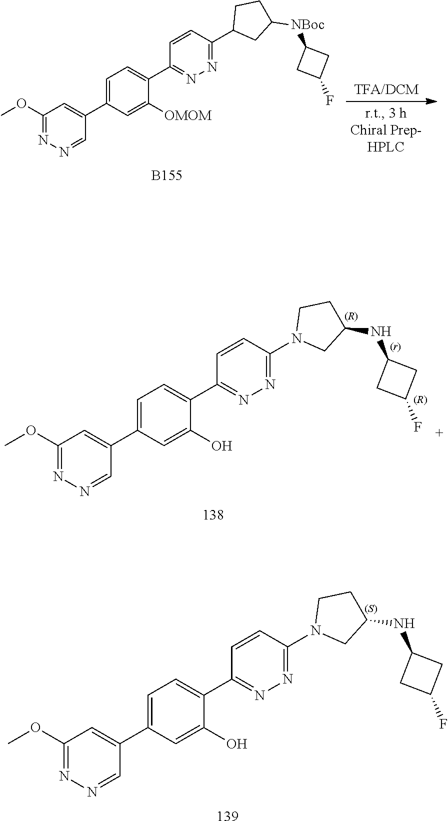

A solution of tert-butyl N-(1-{6-[2-(methoxymethoxy)-4-(6-methoxypyridazin-4-yl)phenyl]pyridazin-3-yl}pyrrolidin-3-yl)-N-[(1r,3r)-3-fluorocyclobutyl]carbamate (250 mg, 0.431 mmol, 1 equiv) in DCM (3 mL) was treated with TFA (1 mL) for 3 h at room temperature. The resulting mixture was concentrated under reduced pressure. The crude product was purified by Prep-Chiral-HPLC (Condition 11, Gradient 2) to afford 5-(6-methoxypyridazin-4-yl)-2-[6-(3-{[(1r,3r)-3-fluorocyclobutyl]amino}pyrrolidin-1-yl)pyridazin-3-yl]phenol (40 mg, 21.28%) as a solid. 5-(6-methoxypyridazin-4-yl)-2-[6-(3-{[(1r,3r)-3-fluorocyclobutyl]amino}pyrrolidin-1-yl)pyridazin-3-yl]phenol (40 mg, 0.092 mmol, 1 equiv) was purified under identical conditions to afford 5-(6-methoxypyridazin-4-yl)-2-{6-[(3R)-3-{[(1r,3r)-3-fluorocyclobutyl]amino}pyrrolidin-1-yl]pyridazin-3-yl}phenol (6.9 mg, 17%) as a solid and 5-(6-methoxypyridazin-4-yl)-2-{6-[(3S)-3-{[(1r,3r)-3-fluorocyclobutyl]amino}pyrrolidin-1-yl]pyridazin-3-yl}phenol (9.1 mg, 22%) as a solid. Compound 138: LCMS:(ES, m/z):437′H NMR (400 MHz, DMSO-d₆) δ 14.11 (s, 1H), 9.35 (d, J=1.9 Hz, 1H), 8.31 (d, J=9.9 Hz, 1H), 8.05 (d, J=8.3 Hz, 1H), 7.56 (d, J=1.9 Hz, 1H), 7.51-7.45 (m, 2H), 7.19 (d, J=9.8 Hz, 1H), 5.19 (dtt, J=56.9, 6.5, 3.5 Hz, 1H), 4.08 (s, 3H), 3.72-3.60 (m, 2H), 3.50 (dd, J=10.1, 7.1 Hz, 2H), 3.39 (t, J=5.6 Hz, 1H), 3.29 (t, J=5.6 Hz, 1H), 2.40-2.29 (m, 2H), 2.21-2.08 (m, 3H), 1.85 (dt, J=12.6, 6.6 Hz, 1H). Compound 139: LCMS:(ES, m/z):437′H NMR: (400 MHz, DMSO-d₆) δ 14.12 (s, 1H), 9.35 (t, J=1.6 Hz, 1H), 8.34-8.27 (m, 1H), 8.04 (d, J=8.2 Hz, 1H), 7.56 (t, J=1.6 Hz, 1H), 7.53-7.44 (m, 2H), 7.22-7.15 (m, 1H), 5.19 (ddq, J=56.9, 6.8, 3.4 Hz, 1H), 4.09 (d, J=1.3 Hz, 3H), 3.73-3.59 (m, 2H), 3.53 (h, J=8.5, 6.6 Hz, 2H), 3.39 (q, J=5.7 Hz, 1H), 3.29 (d, J=11.6 Hz, 1H), 2.39-2.28 (m, 2H), 2.13 (ddt, J=18.7, 13.0, 6.7 Hz, 3H), 1.83 (dq, J=13.2, 6.8 Hz, 1H).

### Example 55: Synthesis of Compound 142, 238, and 290

### Synthesis of Intermediate B]56

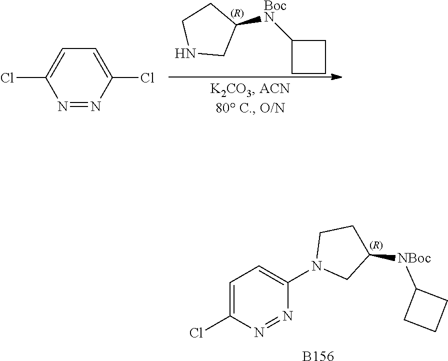

A solution of tert-butyl N-cyclobutyl-N-[(3R)-pyrrolidin-3-yl]carbamate (726.03 mg, 3.021 mmol, 1.5 equiv) in ACN (10 mL) was treated with pyridazine, 3,6-dichloro- (300 mg, 2.014 mmol, 1 equiv) and K₂CO₃ (834.96 mg, 6.042 mmol, 3 equiv) overnight at 80° C. The resulting mixture was filtered and the filter cake was washed with CH₂Cl₂ (3×5 mL). The filtrate was concentrated under reduced pressure. The residue was purified by silica gel column chromatography, eluted with PE/EA (3:2) to afford tert-butyl N-[(3R)-1-(6-chloropyridazin-3-yl)pyrrolidin-3-yl]-N-cyclobutylcarbamate (600 mg, 84%) as a solid.

### Synthesis of Intermediate B]57

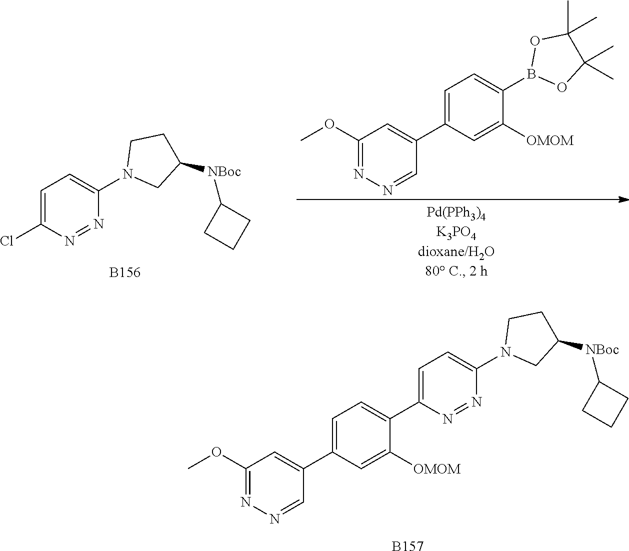

A solution of tert-butyl N-[(3R)-1-(6-chloropyridazin-3-yl)pyrrolidin-3-yl]-N-cyclobutylcarbamate (500 mg, 1.417 mmol, 1 equiv) in dioxane (10 mL)/H₂O (2 mL) was treated with 3-methoxy-5-[3-(methoxymethoxy)-4-(4,4,5,5-tetramethyl-1,3,2-dioxaborolan-2-yl)phenyl]pyridazine (791.17 mg, 2.126 mmol, 1.5 equiv), Pd(PPh₃)4 (163.75 mg, 0.142 mmol, 0.1 equiv) and K₃PO₄ (902.33 mg, 4.251 mmol, 3 equiv) for 2 h at 80° C. under nitrogen atmosphere. The resulting mixture was filtered and the filter cake was washed with EtOAc (3×5 mL). The aqueous layer was extracted with EtOAc (3×2 mL). The combined organic layers were dried over anhydrous Na₂SO₄. After filtration, the filtrate was concentrated under reduced pressure. The residue was purified by silica gel column chromatography, eluted with PE/EA (2:1) to afford tert-butyl N-cyclobutyl-N-[(3R)-1-{6-[2-(methoxymethoxy)-4-(6-methoxypyridazin-4-yl)phenyl]pyridazin-3-yl}pyrrolidin-3-yl]carbamate (650 mg, 82%) as a solid.

### Synthesis of Compound 142

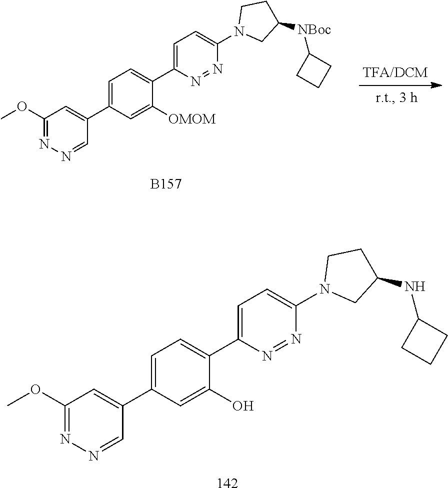

A solution of tert-butyl N-cyclobutyl-N-[(3R)-1-{6-[2-(methoxymethoxy)-4-(6-methoxypyridazin-4-yl)phenyl]pyridazin-3-yl}pyrrolidin-3-yl]carbamate (650 mg, 1.155 mmol, 1 equiv) in TFA (1 mL)DCM (3 mL) was stirred for 3 h at room temperature. The resulting mixture was concentrated under reduced pressure to afford crude product 400 mg. A part of crude product (100 mg) was purified by Chiral-Prep-HPLC (Condition 10, Gradient 3) to afford 2-{6-[(3R)-3-(cyclobutylamino)pyrrolidin-1-yl]pyridazin-3-yl}-5-(6-methoxypyridazin-4-yl)phenol (15.9 mg, 3%) as a solid. LCMS:(ES, m/z): 419 [M+H]^{+ 1}HNMR: (400 MHz, DMSO-d₆) δ 14.14 (s, 1H), 9.36 (d, J=1.9 Hz, 1H), 8.30 (d, J=9.8 Hz, 1H), 8.04 (d, J=8.3 Hz, 1H), 7.56 (d, J=1.9 Hz, 1H), 7.53-7.44 (m, 2H), 7.17 (d, J=9.7 Hz, 1H), 4.08 (s, 3H), 3.63 (dt, J 14.9, 7.5 Hz, 2H), 3.49 (d, J=7.6 Hz, 1H), 3.25 (q, J=8.0, 7.5 Hz, 2H), 2.12 (ddp, J=24.6, 18.7, 6.1 Hz, 4H), 1.82 (dq, J=13.0, 6.7 Hz, 1H), 1.76-1.62 (m, 2H), 1.57 (dtd, J=18.1, 10.1, 7.7 Hz, 2H).

### Synthesis of Compound 238

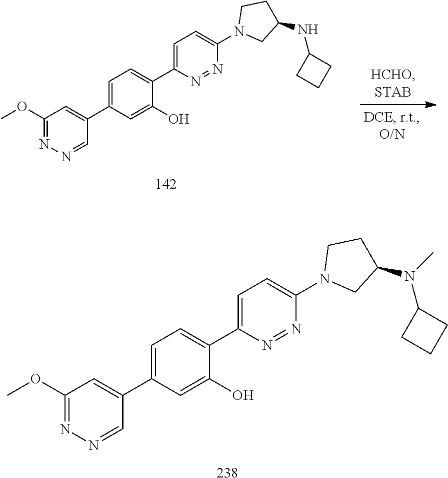 [SMILES: COc1cc(-c2ccc(-c3ccc(N4CC[C@@H](NC5CCC5)C4)nn3)c(O)c2)cnn1 ; "142" ; COc1cc(-c2ccc(-c3ccc(N4CC[C@@H](N(C)C5CCC5)C4)nn3)c(O)c2)cnn1 ; "238"]

A solution of 2-{6-[(3R)-3-(cyclobutylamino)pyrrolidin-1-yl]pyridazin-3-yl}-5-(6-methoxypyridazin-4-yl)phenol (150 mg, 0.358 mmol, 1 equiv) in DCE (7.5 mL, 94.634 mmol, 264.34 equiv) was treated with HCHO (16.14 mg, 0.537 mmol, 1.5 equiv) for 10 min followed by the addition of STAB (227.89 mg, 1.074 mmol, 3 equiv) dropwise at 0° C. The final reaction mixture was stirred overnight at room temperature. The reaction was quenched by the addition of Water (5 mL) at 0° C. The aqueous layer was extracted with EtOAc (3×3 mL). The combined organic layers were dried over anhydrous Na₂SO₄. After filtration, the filtrate was concentrated under reduced pressure. The crude product was purified by Chiral-Prep-HPLC (Condition 10, Gradient 3) to afford 2-{6-[(3R)-3-[cyclobutyl(methyl)amino]pyrrolidin-1-yl]pyridazin-3-yl}-5-(6-methoxypyridazin-4-yl)phenol (16.2 mg, 10%) as a solid. LCMS:(ES, m/z):433 [M+H]⁺¹H NMR (400 MHz, DMSO-d₆) δ 14.09 (s, 1H), 9.35 (d, J=1.9 Hz, 1H), 8.31 (d, J=9.8 Hz, 1H), 8.05 (d, J=8.3 Hz, 1H), 7.56 (d, J=1.9 Hz, 1H), 7.52-7.44 (m, 2H), 7.21 (d, J=9.7 Hz, 1H), 4.08 (s, 3H), 3.71 (s, 2H), 3.42 (td, J=10.4, 7.3 Hz, 1H), 3.28 (dd, J=10.4, 8.3 Hz, 1H), 3.14 (q, J=7.8 Hz, 1H), 3.02 (p, J=8.2 Hz, 1H), 2.10 (s, 4H), 1.99 (dq, J=7.6, 3.9 Hz, 2H), 1.94-1.80 (m, 3H), 1.58 (dtt, J=13.8, 10.6, 5.2 Hz, 2H).

### Synthesis of Compound 290

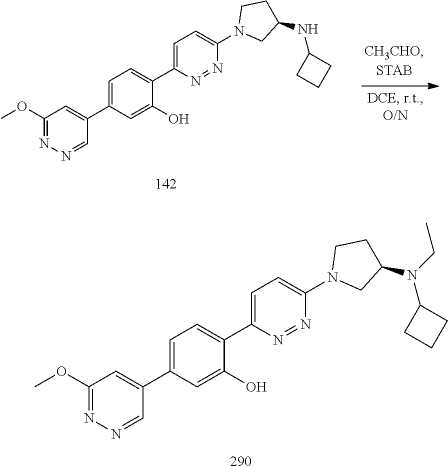 [SMILES: COc1cc(-c2ccc(-c3ccc(N4CC[C@@H](NC5CCC5)C4)nn3)c(O)c2)cnn1 ; "142" ; CCN(C1CCC1)[C@@H]1CCN(c2ccc(-c3ccc(-c4cnnc(OC)c4)cc3O)nn2)C1 ; "290"]

A solution of 2-{6-[(3R)-3-(cyclobutylamino)pyrrolidin-1-yl]pyridazin-3-yl}-5-(6-methoxypyridazin-4-yl)phenol (150 mg, 0.358 mmol, 1 equiv) in DCE (3 mL)was treated with acetaldehyde (23.68 mg, 0.537 mmol, 1.5 equiv) for 10 min followed by the addition of STAB (227.89 mg, 1.074 mmol, 3 equiv) dropwise at 0° C. The final reaction mixture was stirred overnight at room temperature. The reaction was quenched by the addition of Water (5 mL) at 0° C. The aqueous layer was extracted with EtOAc (3×3 mL). The combined organic layers were dried over anhydrous Na₂SO₄. After filtration, the filtrate was concentrated under reduced pressure. The crude product was purified by Chiral-Prep-HPLC (Condition 10, Gradient 3) to afford 2-{6-[(3R)-3-[cyclobutyl(ethyl)amino]pyrrolidin-1-yl]pyridazin-3-yl}-5-(6-methoxypyridazin-4-yl)phenol (22.8 mg, 14%) as a solid. LCMS:(ES, m/z):447 [M+H]⁺¹H NMR (400 MHz, DMSO-d₆) δ 14.19-14.04 (m, 1H), 9.36 (d, J=1.9 Hz, 1H), 8.31 (d, J=9.8 Hz, 1H), 8.05 (d, J=8.3 Hz, 1H), 7.56 (d, J=1.9 Hz, 1H), 7.53-7.43 (m, 2H), 7.20 (d, J=9.7 Hz, 1H), 4.08 (s, 3H), 3.72 (d, J=9.5 Hz, 2H), 3.46-3.37 (m, 2H), 3.22 (ddd, J=19.4, 11.1, 5.8 Hz, 2H), 2.65 (qd, J=6.9, 3.7 Hz, 2H), 2.14 (dt, J=12.4, 6.3 Hz, 1H), 2.08-1.97 (m, 2H), 1.88 (p, J=10.0 Hz, 3H), 1.56 (ddd, J=18.2, 10.3, 7.2 Hz, 2H), 0.94 (t, J=7.1 Hz, 3H).

### Example 56: Synthesis of Compounds 148 and 270

### Synthesis of Intermediate

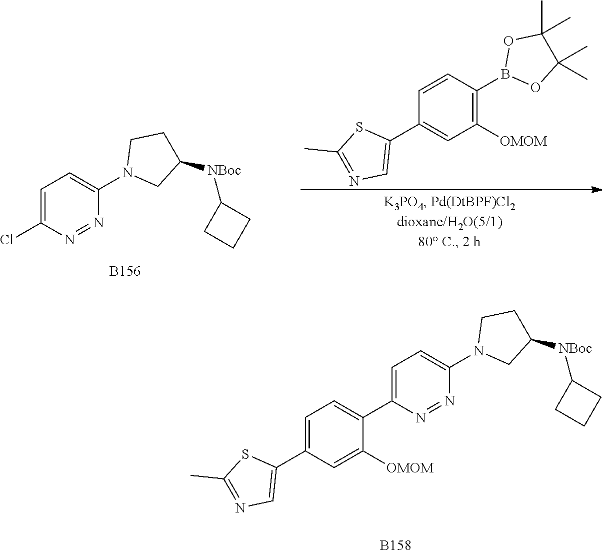

To a solution of tert-butyl N-[(3R)-1-(6-chloropyridazin-3-yl) pyrrolidin-3-yl]-N-cyclobutylcarbamate (400 mg, 1.134 mmol, 1.0 equiv) and 5-[3-(methoxymethoxy)-4-(4,4,5,5-tetramethyl-1,3,2-dioxaborolan-2-yl) phenyl]-2-methyl-1,3-thiazole (491.43 mg, 1.361 mmol, 1.2 equiv) in 1,4-dioxane (5 mL) and H₂O (1 mL) were added K₃PO₄ (601.56 mg, 2.835 mmol, 2.5 equiv) and Pd(DtBPF)Cl₂ (73.88 mg, 0.113 mmol, 0.1 equiv). After stirring for 2 h at 80° C. under a nitrogen atmosphere, the resulting mixture was extracted with EtOAc (3×50 mL). The combined organic layers were washed with water (3×20 mL), dried over anhydrous Na₂SO₄.

After filtration, the filtrate was concentrated under reduced pressure. The residue was purified by silica gel column chromatography, eluted with CH₂Cl₂/MeOH (10:1) to afford tert-butyl N-cyclobutyl-N-[(3R)-1-{6-[2-(methoxymethoxy)-4-(2-methyl-1,3-thiazol-5-yl) phenyl]pyridazin-3-yl}pyrrolidin-3-yl]carbamate (220 mg, 32%) as a solid.

### Synthesis of Compound 148

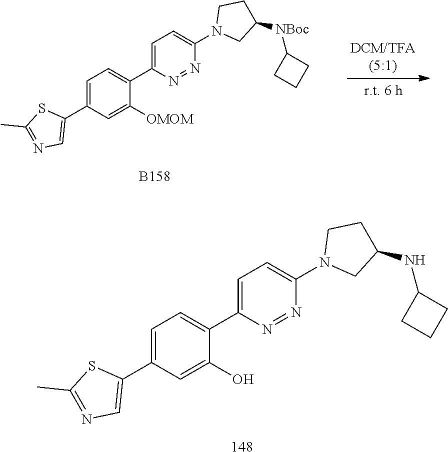

A solution of tert-butyl N-cyclobutyl-N-[(3R)-1-{6-[2-(methoxymethoxy)-4-(2-methyl-1,3-thiazol-5-yl)phenyl]pyridazin-3-yl}pyrrolidin-3-yl]carbamate (220 mg, 0.399 mmol, 1.0 equiv) in DCM (3 mL) was treated with TFA (600 uL) for 6 h at room temperature under nitrogen atmosphere. The resulting mixture was concentrated under reduced pressure. The crude product was purified by Chiral-Prep-HPLC (Condition 8, Gradient 1) to afford 2-{6-[(3R)-3-(cyclobutylamino) pyrrolidin-1-yl]pyridazin-3-yl}-5-(2-methyl-1,3-thiazol-5-yl) phenol (11.6 mg, 7%) as a solid. LCMS:(ES, m/z):408′HNMR: (400 MHz, DMSO-d₆) δ 10.53 (s, 1H), 8.16 (s, 1H), 7.81 (d, J=9.5 Hz, 1H), 7.75-7.63 (m, 2H), 7.45 (dd, J=8.2, 1.8 Hz, 1H), 6.91 (d, J=9.5 Hz, 1H), 3.69-3.53 (m, 2H), 3.51-3.42 (m, 1H), 3.38 (d, J=5.7 Hz, 1H), 3.22 (d, J=4.3 Hz, 2H), 2.66 (s, 3H), 2.11 (tp, J=18.9, 6.4, 5.3 Hz, 4H), 1.80 (dq, J=12.9, 6.8 Hz, 1H), 1.76-1.63 (m, 2H), 1.63-1.49 (m, 2H).

### Synthesis of Compound 270

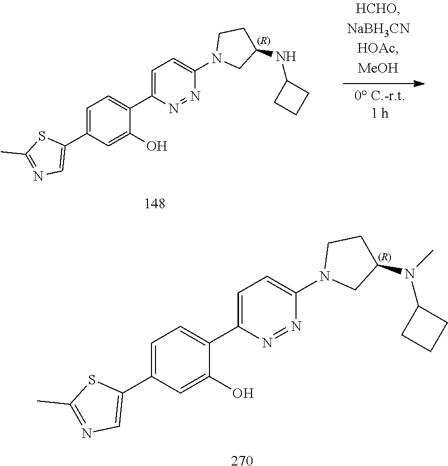

A solution of 2-{6-[(3R)-3-(cyclobutylamino) pyrrolidin-1-yl]pyridazin-3-yl}-5-(2-methyl-1,3-thiazol-5-yl) phenol (120 mg, 0.294 mmol, 1.0 equiv) in MeOH (3 mL) was treated with HOAc (88.41 mg, 1.470 mmol, 5.0 equiv) for 0.5 h at room temperature under nitrogen atmosphere followed by the addition of NaBH₃CN (55.51 mg, 0.882 mmol, 3.0 equiv) at 0° C. The resulting mixture was stirred for 0.5 h at room temperature under nitrogen atmosphere. The reaction was quenched by the addition of Water (3 mL) at room temperature. The resulting mixture was extracted with EtOAc (3×20 mL). The combined organic layers were washed with water (3×20 mL), dried over anhydrous Na₂SO₄. After filtration, the filtrate was concentrated under reduced pressure. The crude product was purified by Chiral-Prep-HPLC (Condition 8, Gradient 1) to afford 2-{6-[(3R)-3-[cyclobutyl(methyl)amino]pyrrolidin-1-yl]pyridazin-3-yl}-5-(2-methyl-1,3-thiazol-5-yl) phenol (21.1 mg, 17%) as a solid. LCMS:(ES, m/z):422′H NMR (400 MHz, DMSO-d₆) δ10.06 (s, 1H), 8.15 (s, 1H), 7.82 (d, J=9.5 Hz, 1H), 7.72 (d, J=8.2 Hz, 1H), 7.67 (d, J=1.8 Hz, 1H), 7.44 (d, J=8.3 Hz, 1H), 6.94 (d, J=9.5 Hz, 1H), 3.69 (q, J 9.1, 8.5 Hz, 2H), 3.41 (dt, J=10.0, 4.9 Hz, 1H), 3.25 (d, J=9.6 Hz, 1H), 3.14 (q, J=7.7 Hz, 1H), 3.01 (q, J=8.7, 8.2 Hz, 1H), 2.66 (s, 3H), 2.10 (m, 4H), 2.00 (dt, J=7.1, 3.5 Hz, 2H), 1.89 (m, J=9.5 Hz, 3H), 1.58 (qt, J=10.9, 5.3 Hz, 2H).

### Example 57: Synthesis of Compound 149

### Synthesis of Intermediate B159

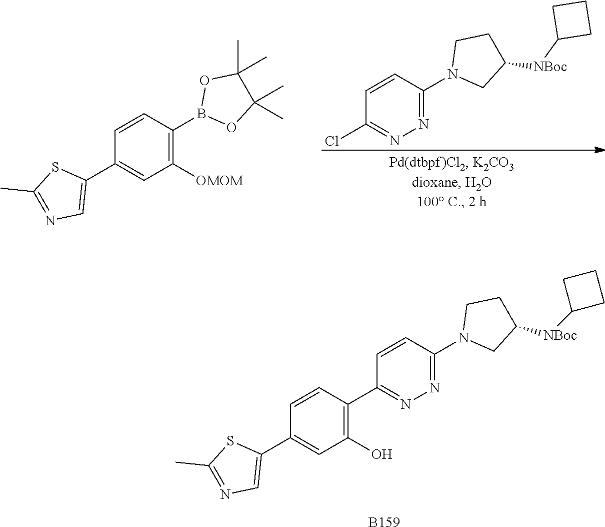

To a solution of 5-[3-(methoxymethoxy)-4-(4,4,5,5-tetramethyl-1,3,2-dioxaborolan-2-yl)phenyl]-2-methyl-1,3-thiazole (200 mg, 0.554 mmol, 1.00 equiv) and tert-butyl N-[(3S)-1-(6-chloropyridazin-3-yl)pyrrolidin-3-yl]-N-cyclobutylcarbamate (214.88 mg, 0.609 mmol, 1.1 equiv) in 1,4-dioxane (0.4 mL) and H₂O (2 mL) were added Pd(dtbpf)Cl₂ (18.4 mg, 0.028 mmol, 0.05 equiv) and K₂CO₃ (229.54 mg, 1.662 mmol, 3 equiv). After stirring for 2 h at 100° C. under a nitrogen atmosphere, the resulting mixture was concentrated under reduced pressure. The residue was purified by silica gel column chromatography, eluted with PE/EA (3:7) to afford tert-butyl N-cyclobutyl-N-[(3S)-1-{6-[2-hydroxy-4-(2-methyl-1,3-thiazol-5-yl)phenyl]pyridazin-3-yl}pyrrolidin-3-yl]carbamate (160 mg, 57%) as a solid.

### Synthesis of Compound 149

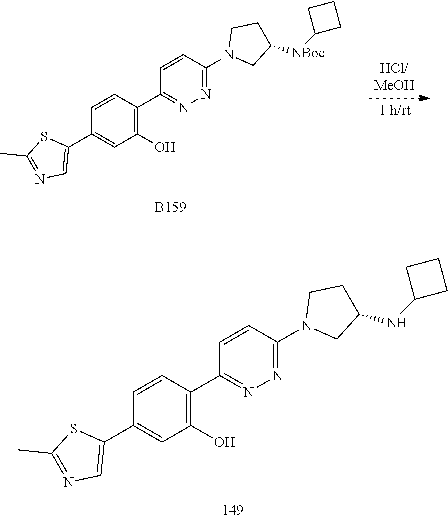 [SMILES: "B159" ; Cc1ncc(-c2ccc(-c3ccc(N4CC[C@H](N(C(=O)OC(C)(C)C)C5CCC5)C4)nn3)c(O)c2)s1 ; Cc1ncc(-c2ccc(-c3ccc(N4CC[C@H](NC5CCC5)C4)nn3)c(O)c2)s1 ; "149"]

To a solution of tert-butyl N-cyclobutyl-N-[(3S)-1-{6-[2-hydroxy-4-(2-methyl-1,3-thiazol-5-yl)phenyl]pyridazin-3-yl}pyrrolidin-3-yl]carbamate (40 mg, 0.079 mmol, 1 equiv) in HCl(gas) in 1,4-dioxane (1 mL) and MeOH (1 mL). After stirring for 1 h at 25° C. under a nitrogen atmosphere, the resulting mixture was concentrated under reduced pressure. The crude product was purified by Chiral-Prep-HPLC (Condition 8, Gradient 1) to afford 2-{6-[(3S)-3-(cyclobutylamino) pyrrolidin-1-yl]pyridazin-3-yl}-5-(2-methyl-1,3-thiazol-5-yl)phenol (6.0 mg, 19%) as a solid. LCMS (ES, m/z): 407.9 [M+H]^{+ 1}H NMR (400 MHz, DMSO-d₆) δ 10.28 (s, 1H), 8.16 (s, 1H), 7.81 (d, J=9.5 Hz, 1H), 7.75-7.62 (m, 2H), 7.45 (d, J=8.2 Hz, 1H), 6.90 (d, J=9.5 Hz, 1H), 3.61 (tt, J=14.7, 6.6 Hz, 2H), 3.52-3.42 (m, 1H), 3.34-3.37 (m, 1H), 3.23 (d, J=8.5 Hz, 2H), 2.66 (s, 3H), 2.11 (tdd, J=18.7, 10.8, 5.0 Hz, 4H), 1.80 (dd, J=12.5, 6.5 Hz, 1H), 1.73-1.48 (m, 4H).

### Example 58: Synthesis of Compounds 151, 152, and 153

### Synthesis of Intermediate B160

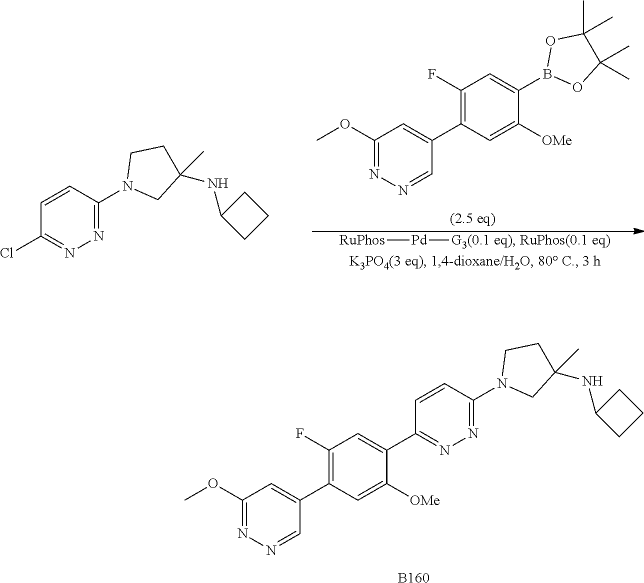

A solution of 1-(6-chloropyridazin-3-yl)-N-(1-methylcyclobutyl)pyrrolidin-3-amine (200 mg, 0.750 mmol, 1.00 equiv), 5-[2-fluoro-5-methoxy-4-(4,4,5,5-tetramethyl-1,3,2-dioxaborolan-2-yl)phenyl]-3-methoxypyridazine (675.09 mg, 1.875 mmol, 2.5 equiv) in 1,4-dioxane (10 mL) and H₂O (2 mL) was treated with K₃PO₄ (477.41 mg, 2.250 mmol, 3 equiv) and RuPhos Palladacycle Gen.3 (62.7 mg, 0.075 mmol, 0.1 equiv) and RuPhos (34.98 mg, 0.075 mmol, 0.1 equiv) for 3 h at 80° C. under nitrogen atmosphere. The mixture was allowed to cool down to room temperature. The reaction was quenched with H₂O at room temperature. The resulting mixture was extracted with EA (2×10 mL). The combined organic layers were washed with brine Solution (1×10 mL), dried over anhydrous Na₂SO₄. After filtration, the filtrate was concentrated under reduced pressure.

The residue was purified by silica gel column chromatography, eluted with DCM:MeOH (9:1) to afford 1-{6-[5-fluoro-2-methoxy-4-(6-methoxypyridazin-4-yl)phenyl]pyridazin-3-yl}-N-(1-methylcyclobutyl)pyrrolidin-3-amine (165 mg, 38%) as a solid. LCMS (ES, m/z): 465 [M+H]⁺

### Synthesis of Intermediate B161

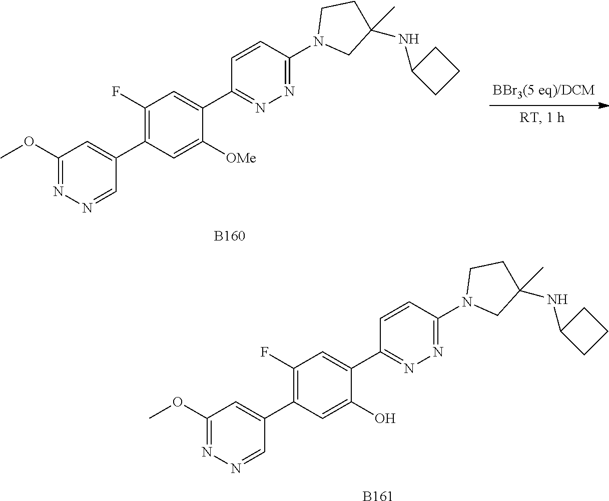

A solution of N-cyclobutyl-1-{6-[5-fluoro-2-methoxy-4-(6-methoxypyridazin-4-yl)phenyl]pyridazin-3-yl}-3-methylpyrrolidin-3-amine (165 mg, 0.284 mmol, 1 equiv, 80%) in DCM (10 mL) was stirred for BBr₃ (444.91 mg, 1.775 mmol, 5 equiv) at room temperature. The mixture was stirred 1 h at room temperature. The reaction was quenched by the addition of MeOH (5 mL) at 0° C. The resulting mixture was concentrated under reduced pressure. The mixture was basified to pH 8 with saturated NaHCO₃ (aq.). The aqueous layer was extracted with DCM (3×10 mL). The combined organic layers were washed with brine Solution (1×15 mL), dried over anhydrous Na₂SO₄. After filtration, the filtrate was concentrated under reduced pressure. The crude product was purified by Prep-HPLC (Condition 6, Gradient 1) to afford 2-{6-[3-(cyclobutylamino)-3-methylpyrrolidin-1-yl]pyridazin-3-yl}-4-fluoro-5-(6-methoxypyridazin-4-yl)phenol (23 mg, 18%) as a solid. LCMS (ES, m/z): 451 [M+H]⁺

### Synthesis of Compounds 152 and 153

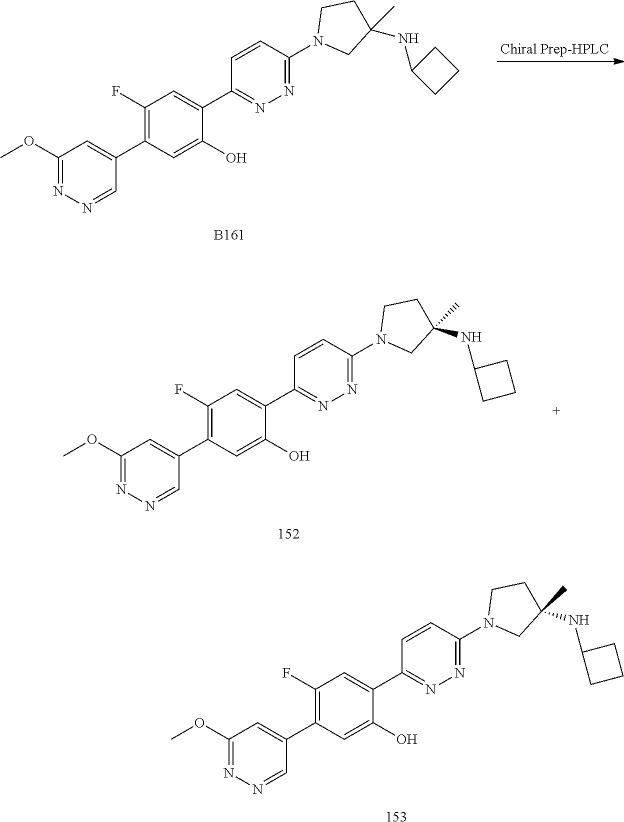 [SMILES: "B161" ; COc1cc(-c2cc(O)c(-c3ccc(N4CCC(C)(NC5CCC5)C4)nn3)cc2F)cnn1 ; COc1cc(-c2cc(O)c(-c3ccc(N4CC[C@@](C)(NC5CCC5)C4)nn3)cc2F)cnn1 ; "152" ; COc1cc(-c2cc(O)c(-c3ccc(N4CC[C@](C)(NC5CCC5)C4)nn3)cc2F)cnn1 ; "153"]

The residue was purified by Prep-Chiral-HPLC (Condition 12, Gradient 1) to afford first eluting 2-{6-[(3R)-3-(cyclobutylamino)-3-methylpyrrolidin-1-yl]pyridazin-3-yl}-4-fluoro-5-(6-methoxypyridazin-4-yl)phenol (1.2 mg, 5.12%) as a and second eluting 2-{6-[(3S)-3-(cyclobutylamino)-3-methylpyrrolidin-1-yl]pyridazin-3-yl}-4-fluoro-5-(6-methoxypyridazin-4-yl)phenol (1.5 mg, 5%) as a solid. Compound 152: LCMS (ES, m/z): 451 [M+H] ⁺¹H NMR (400 MHz, DMSO-d₆) δ 13.73 (s, 1H), 8.25 (d, J=9.8 Hz, 1H), 8.16 (s, 1H), 7.92 (d, J=12.7 Hz, 1H), 7.29 (d, J=6.8 Hz, 1H), 7.14 (d, J=9.7 Hz, 1H), 3.62 (s, 1H), 3.54 (s, 1H), 3.44 (s, 1H), 3.32 (s, 1H), 3.28 (s, 1H), 2.70 (s, 3H), 2.13 (m, 2H), 1.99 (dt, J=12.8, 6.7 Hz, 1H), 1.81 (dt, J=12.4, 7.0 Hz, 1H), 1.70 (q, J 9.4 Hz, 2H), 1.59-1.46 (m, 2H), 1.21 (s, 3H). Compound 153: LCMS (ES, m/z): 451 [M+H] ⁺¹H NMR (400 MHz, DMSO-d₆) δ 13.73 (s, 1H), 8.25 (d, J=9.8 Hz, 1H), 8.16 (s, 1H), 7.92 (d, J=12.7 Hz, 1H), 7.29 (d, J=6.8 Hz, 1H), 7.14 (d, J=9.8 Hz, 1H), 3.61 (s, 1H), 3.54 (s, 1H), 3.43 (s, 1H), 3.32 (s, 1H), 3.28 (s, 1H), 2.71 (s, 3H), 2.13 (m, 2H), 1.99 (dt, J=13.0, 6.9 Hz, 1H), 1.81 (dt, J=12.6, 7.2 Hz, 1H), 1.76-1.67 (m, 2H), 1.54-1.48 (m, 2H), 1.21 (s, 3H).

### Example 59: Synthesis of Compounds 154, 155, and 156

### Synthesis of Intermediate B162

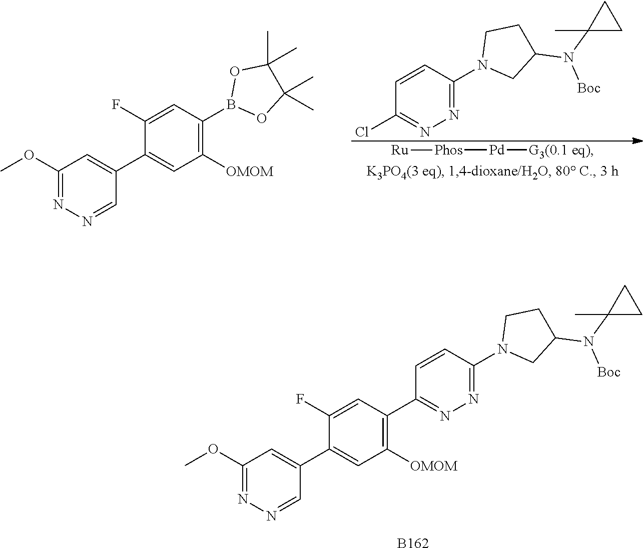

A solution of 5-[2-fluoro-5-(methoxymethoxy)-4-(4,4,5,5-tetramethyl-1,3,2-dioxaborolan-2-yl) phenyl]-3-methoxypyridazine (210 mg, 0.538 mmol, 1 equiv) and tert-butyl N-[1-(6-chloropyridazin-3-yl)pyrrolidin-3-yl]-N-(1-methylcyclopropyl)carbamate (189.89 mg, 0.538 mmol, 1 equiv) and RuPhos Palladacycle Gen.3 (45.01 mg, 0.054 mmol, 0.1 equiv) and K₃PO₄ (342.7 mg, 1.614 mmol, 3 equiv) in 1,4-dioxane/H₂O=5:1(6 mL) was stirred for 3 h at 80° C. under nitrogen atmosphere. The resulting mixture was concentrated under reduced pressure. The residue was purified by silica gel column chromatography, eluted with CH₂Cl₂/MeOH (10:1) to afford tert-butyl N-(1-{6-[5-fluoro-2-(methoxymethoxy)-4-(6-methoxypyridazin-4-yl)phenyl]pyridazin-3-yl}pyrrolidin-3-yl)-N-(1-methylcyclopropyl)carbamate (250 mg, 80%) as a solid.

### Synthesis of Compounds 154-156

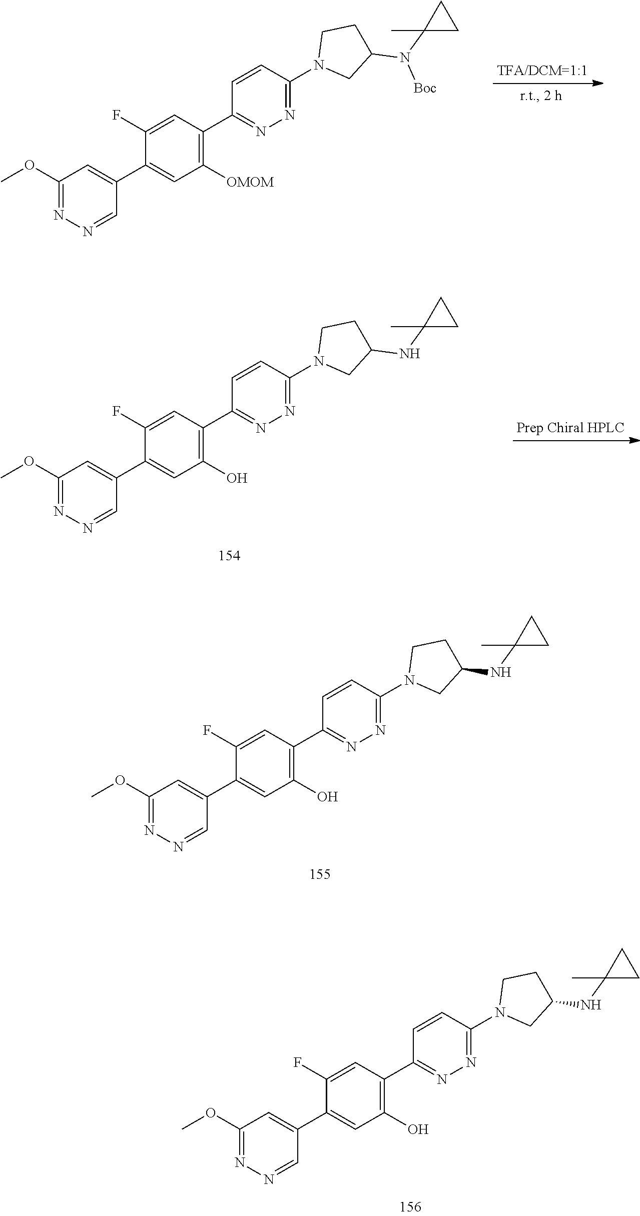

A solution of tert-butyl N-(1-{6-[5-fluoro-2-(methoxymethoxy)-4-(6-methoxypyridazin-4-yl) phenyl]pyridazin-3-yl}pyrrolidin-3-yl)-N-(1-methylcyclopropyl)carbamate (250 mg, 0.431 mmol, 1 equiv) and TFA (490.92 mg, 4.310 mmol, 10 equiv) in DCM(10 mL) was stirred for 2 h at room temperature under air atmosphere. The mixture was basified to pH 9 with saturated NaHCO₃ (aq.).Column: CHIRAL ART Cellulose-SB, 2*25 cm, 5 m; Mobile Phase A: HEX: DCM=3: 1(0.1% DEA)-HPLC, Mobile Phase B: MeOH-HPLC; Flow rate: 20 mL/min; Gradient: 10% B to 10% B in 11 min; Wave Length: UV 254/220 nm; RTl(min): 8.2; RT2(min): 9.31; Sample Solvent: DCM-HPLC; Injection Volume: 0.2 mL; Number Of Runs: to afford 4-fluoro-5-(6-methoxypyridazin-4-yl)-2-(6-{3-[(1-methylcyclopropyl)amino]pyrrolidin-1-yl}pyridazin-3-yl)phenol (180 mg, 92%) as a solid. The crude product (180 mg) was purified by Prep-HPLC with the following conditions Column: CHIRAL ART Cellulose-SB, 4.6*100 mm, 3 m; Mobile Phase A: Hex: DCM=3: 1(0.1% DEA): MeOH=90: 10; Flow rate: 1 mL/min; Gradient: 0% B to 0% B; Injection Volume: Sul mL to afford 4-fluoro-5-(6-methoxypyridazin-4-yl)-2-{6-[(3R)-3-[(1-methylcyclopropyl)amino]pyrrolidin-1-yl]pyridazin-3-yl}phenol (15.4 mg, 8%) as a solid and 4-fluoro-5-(6-methoxypyridazin-4-yl)-2-{6-[(3S)-3-[(1-methylcyclopropyl)amino]pyrrolidin-1-yl]pyridazin-3-yl}phenol (18.8 mg, 9%) as a solid.

Compound 155: LCMS (ES, m/z): 437 [M+H]^{+ 1}H NMR (400 MHz, DMSO-d₆) δ 13.80 (s, 1H), 9.16 (t, J=1.9 Hz, 1H), 8.30 (d, J=9.9 Hz, 1H), 7.99 (d, J=12.5 Hz, 1H), 7.44 (dd, J=1.9, 1.0 Hz, 1H), 7.29 (d, J=6.9 Hz, 1H), 7.18 (d, J=9.8 Hz, 1H), 4.09 (s, 3H), 3.72 (d, J=9.7 Hz, 2H), 3.69-3.57 (m, 1H), 3.50 (d, J=9.3 Hz, 1H), 3.29 (s, 1H), 2.45 (s, 1H), 2.13 (dq, J 12.5, 6.2 Hz, 1H), 1.88 (dq, J=13.5, 7.2 Hz, 1H), 1.26 (s, 3H), 0.47 (q, J=10.8, 10.2 Hz, 2H), 0.33 (s, 2H). Compound 156: LCMS (ES, m/z): 437 [M+H]^{+ 1}H NMR (400 MHz, DMSO-d₆) δ 13.83 (s, 1H), 9.17 (t, J=1.9 Hz, 1H), 8.31 (d, J=9.8 Hz, 1H), 8.01 (d, J=12.5 Hz, 1H), 7.47(dd, J=1.9, 1.0 Hz, 1H), 7.30 (d, J=7.0 Hz, 1H), 7.19 (d, J=9.8 Hz, 1H), 4.09 (s, 3H), 3.73(d, J=9.7 Hz, 2H), 3.69-3.57 (m, 1H), 3.51 (s, 1H), 3.29 (s, 1H), 2.45 (s, 1H), 1.88 (dd, J=12.6, 6.6 Hz, 1H), 1.26 (s, 3H), 0.46 (q, J=10.4 Hz, 2H), 0.33 (s, 2H).

### Example 60: Synthesis of Compound 164

### Synthesis of Intermediate B163

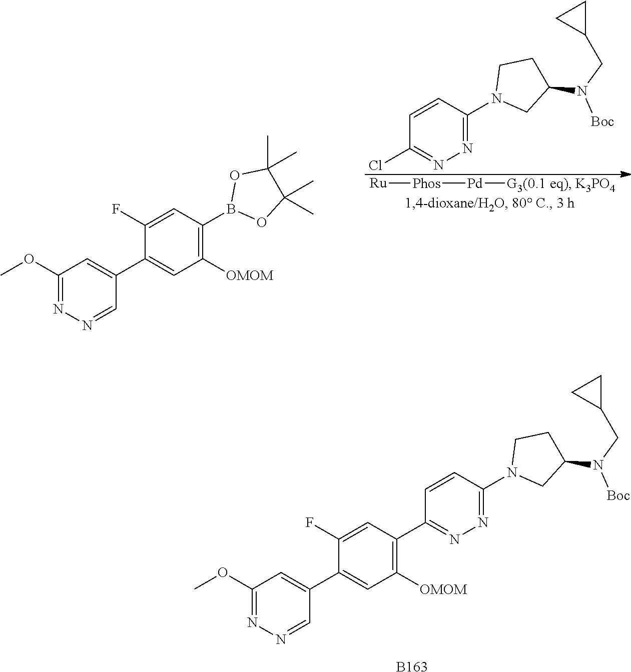

A solution of 5-[2-fluoro-5-(methoxymethoxy)-4-(4,4,5,5-tetramethyl-1,3,2-dioxaborolan-2-yl)phenyl]-3-methoxypyridazine (110 mg, 0.282 mmol, 1 equiv) and tert-butyl N-[(3R)-1-(6-chloropyridazin-3-yl)pyrrolidin-3-yl]-N-(cyclopropylmethyl)carbamate (99.47 mg, 0.282 mmol, 1 equiv) and RuPhos Palladacycle Gen.3 (23.58 mg, 0.028 mmol, 0.1 equiv) and K₃PO₄ (179.51 mg, 0.846 mmol, 3 equiv) in 1,4-dioxane/H₂O=5:1(6 mL) was stirred for 3 h at 80° C. under nitrogen atmosphere. The resulting mixture was concentrated under reduced pressure. The residue was purified by silica gel column chromatography, eluted with CH₂Cl₂/MeOH (10:1) to afford tert-butyl N-(cyclopropylmethyl)-N-[(3R)-1-{6-[5-fluoro-2-(methoxymethoxy)-4-(6-methoxypyridazin-4-yl)phenyl]pyridazin-3-yl}pyrrolidin-3-yl]carbamate (120 mg, 73%) as a solid.

### Synthesis of Compound 164

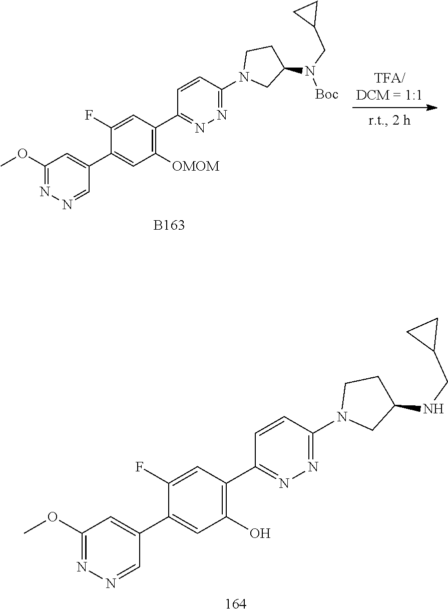

A solution of tert-butyl N-(cyclopropylmethyl)-N-[(3R)-1-{6-[5-fluoro-2-(methoxymethoxy)-4-(6-methoxypyridazin-4-yl)phenyl]pyridazin-3-yl}pyrrolidin-3-yl]carbamate (120 mg, 0.207 mmol, 1 equiv) in DCM/TFA=1:1(20 mL) was stirred for 2 h at room temperature under air atmosphere. The mixture was basified to pH 9 with saturated NaHCO₃ (aq.) and purified by Prep-HPLC (Condition 6, Gradient 1) to afford 2-{6-[(3R)-3-[(cyclopropylmethyl)amino]pyrrolidin-1-yl]pyridazin-3-yl}-4-fluoro-5-(6-methoxypyridazin-4-yl)phenol (10.2 mg, 11%) as a solid. LCMS (ES, m/z): 437 [M+H]^{+ 1}H NMR (400 MHz, DMSO-d₆) δ 13.83 (s, 1H), 9.17 (t, J=1.9 Hz, 1H), 8.32 (d, J=9.7 Hz, 1H), 8.01 (d, J=12.4 Hz, 1H), 7.47-7.42 (m, 1H), 7.30 (d, J=6.9 Hz, 1H), 7.20 (d, J=9.8 Hz, 1H), 4.09 (s, 3H), 3.67 (d, J=1.9 Hz, 2H), 3.54 (d, J=1.6 Hz, 2H), 3.47 (s, 1H), 2.45 (s, 3H), 2.16-2.09 (m, 2H), 0.89 (s, 1H), 0.45-0.37 (m, 2H), 0.13 (d, J=4.7 Hz, 2H).

### Example 61: Synthesis of Compound 165

### Synthesis of Intermediate B164

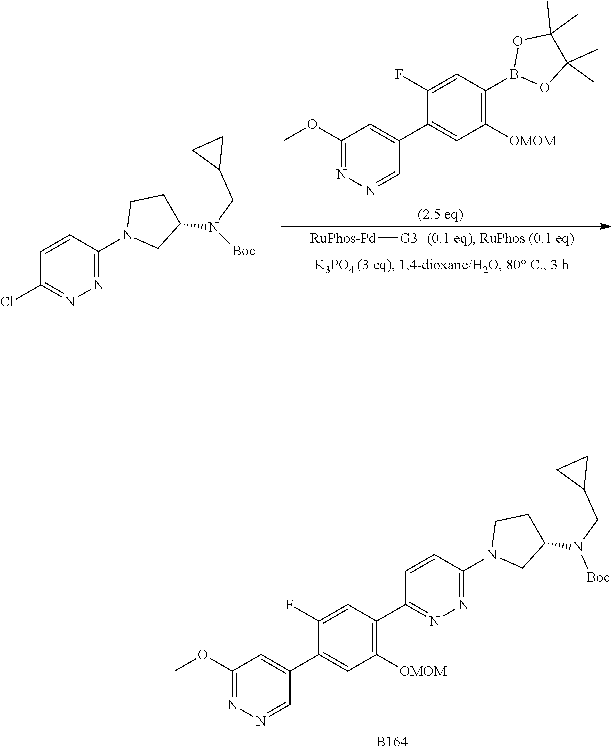

A solution of tert-butyl N-[(3S)-1-(6-chloropyridazin-3-yl)pyrrolidin-3-yl]-N-(cyclopropylmethyl)carbamate (120 mg, 0.340 mmol, 1 equiv), 5-[2-fluoro-5-(methoxymethoxy)-4-(4,4,5,5-tetramethyl-1,3,2-dioxaborolan-2-yl)phenyl]-3-methoxypyridazine (331.76 mg, 0.850 mmol, 2.5 equiv) in 1,4-dioxane (5 mL) and H₂O (1 mL) was treated with K₃PO₄ (216.56 mg, 1.020 mmol, 3 equiv) and RuPhos Palladacycle Gen.3 (28.44 mg, 0.034 mmol, 0.1 equiv) and RuPhos (15.87 mg, 0.034 mmol, 0.1 equiv) for 3 h at 80 degrees C. under nitrogen atmosphere. The mixture was allowed to cool down to room temperature. The reaction was quenched with H₂O at room temperature. The resulting mixture was extracted with EA (2×10 mL). The combined organic layers were washed with brine (1×10 mL), dried over anhydrous Na₂SO₄. After filtration, the filtrate was concentrated under reduced pressure. The residue was purified by silica gel column chromatography, eluted with DCM:MeOH (9:1) to afford tert-butyl N-(cyclopropylmethyl)-N-[(3S)-1-{6-[5-fluoro-2-(methoxymethoxy)-4-(6-methoxypyridazin-4-yl)phenyl]pyridazin-3-yl}pyrrolidin-3-yl]carbamate (96 mg, 49%) as a solid. LCMS (ES, m/z): 581 [M+H]⁺

### Synthesis of Compound 165

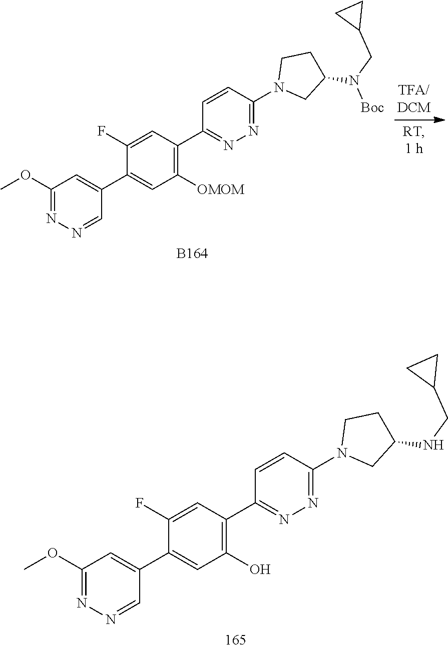

To a stirred solution of tert-butyl N-(cyclopropylmethyl)-N-[(3S)-1-{6-[5-fluoro-2-(methoxymethoxy)-4-(6-methoxypyridazin-4-yl)phenyl]pyridazin-3-yl}pyrrolidin-3-yl]carbamate (96 mg, 0.165 mmol, 1 equiv) in DCM (2 mL) was added TFA (3 mL) dropwise at room temperature. The mixture was stirred 1 h at room temperature. The reaction liquid was concentrated under reduced pressure. The crude product was purified by reverse phase flash (Condition 6, Gradient 1) to afford 2-{6-[(3S)-3-[(cyclopropylmethyl)amino]pyrrolidin-1-yl]pyridazin-3-yl}-4-fluoro-5-(6-methoxypyridazin-4-yl)phenol (16.8 mg, 23%) as a solid. LCMS (ES, m/z): 439 [M+H]+H NMR (400 MHz, DMSO-d₆) δ 13.83 (s, 1H), 9.17 (t, J=1.9 Hz, 1H), 8.32 (d, J=9.7 Hz, 1H), 8.01 (d, J=12.4 Hz, 1H), 7.47-7.42 (m, 1H), 7.30 (d, J=6.9 Hz, 1H), 7.20 (d, J=9.8 Hz, 1H), 4.09 (s, 3H), 3.67 (d, J=1.9 Hz, 2H), 3.54 (d, J=1.6 Hz, 1H), 3.47 (s, 1H), 2.45 (s, 2H), 2.16 (m, 1H), 1.90 (m, 1H), 0.89 (s, 1H), 0.45-0.37 (m, 2H), 0.13 (d, J=4.7 Hz, 2H).

### Example 62: Synthesis of Compound 166, 167, and 168

### Synthesis of Intermediate B165

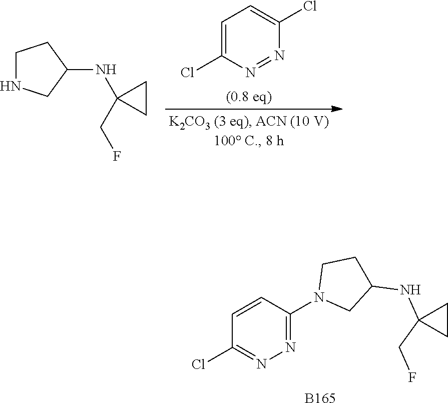

A solution of N-[1-(fluoromethyl)cyclopropyl]pyrrolidin-3-amine (200 mg, 1.264 mmol, 1 equiv) and pyridazine, 3,6-dichloro- (150.65 mg, 1.011 mmol, 0.8 equiv) and K₂CO₃ (524.10 mg, 3.792 mmol, 3 equiv) in ACN (2 mL) as stirred for 8 h at 100 degrees C. The mixture was allowed to cool down to room temperature. The reaction was quenched with H₂O at room temperature. The resulting mixture was extracted with EA (2×10 mL). The combined organic layers were washed with brine Solution (1×10 mL), dried over anhydrous Na₂SO₄. After filtration, the filtrate was concentrated under reduced pressure. This resulted in 1-(6-chloropyridazin-3-yl)-N-[1-(fluoromethyl)cyclopropyl]pyrrolidin-3-amine (180 mg, 53%) as a solid. LCMS (ES, m/z): 271 [M+H]⁺

### Synthesis of Intermediate B166

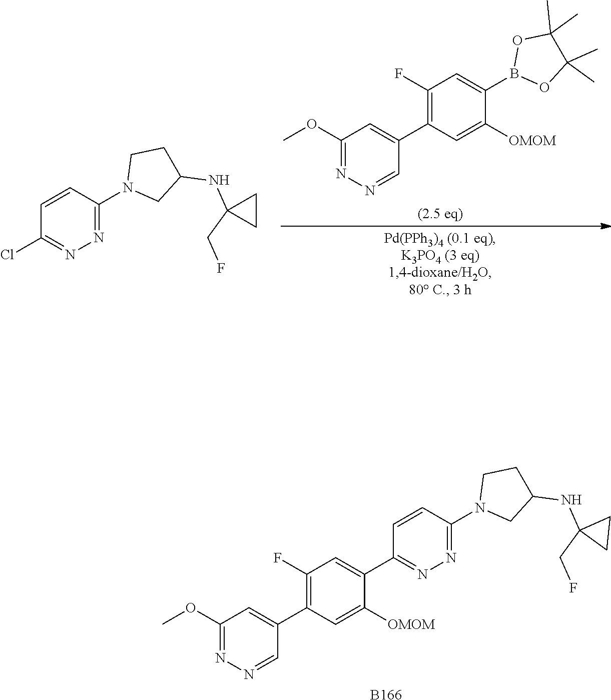

A solution of 1-(6-chloropyridazin-3-yl)-N-[1-(fluoromethyl)cyclopropyl]pyrrolidin-3-amine (180 mg, 0.665 mmol, 1 equiv), 5-[2-fluoro-5-(methoxymethoxy)-4-(4,4,5,5-tetramethyl-1,3,2-dioxaborolan-2-yl)phenyl]-3-methoxypyridazine (648.59 mg, 1.663 mmol, 2.5 equiv) in 1,4-dioxane (5 mL) and H₂O (1 mL) was treated with K₃PO₄ (216.56 mg, 1.020 mmol, 3 equiv) and Pd(PPh₃)₄(76.83 mg, 0.067 mmol, 0.1 equiv) for 3 h at 80 degrees C. under nitrogen atmosphere. The mixture was allowed to cool down to room temperature. The reaction was quenched with H₂O at room temperature. The resulting mixture was extracted with EA (2×10 mL). The combined organic layers were washed with brine (1×10 mL), dried over anhydrous Na₂SO₄. After filtration, the filtrate was concentrated under reduced pressure. The residue was purified by silica gel column chromatography, eluted with DCM:MeOH (9:1) to afford 1-{6-[5-fluoro-2-(methoxymethoxy)-4-(6-methoxypyridazin-4-yl)phenyl]pyridazin-3-yl}-N-[1-(fluoromethyl)cyclopropyl]pyrrolidin-3-amine (120 mg, 36%) as a solid. LCMS (ES, m/z): 499 [M+H]⁺

### Synthesis of Compound 166

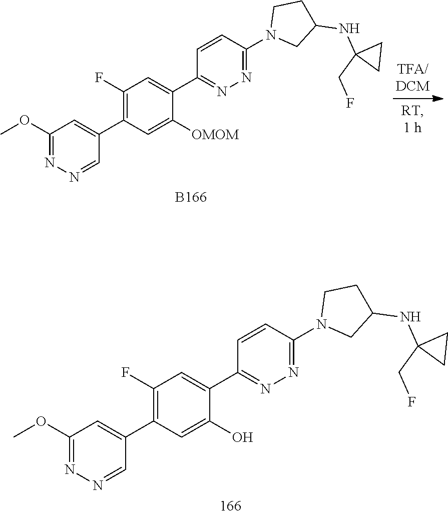

To a stirred solution of 1-{6-[5-fluoro-2-(methoxymethoxy)-4-(6-methoxypyridazin-4-yl)phenyl]pyridazin-3-yl}-N-[1-(fluoromethyl)cyclopropyl]pyrrolidin-3-amine (120 mg, 0.241 mmol, 1 equiv) in DCM (2 mL) was added TFA (3 mL) dropwise at room temperature. The mixture was stirred 1 h at room temperature. The reaction liquid was concentrated under reduced pressure. The crude product was purified by reverse phase flash (Condition 7, Gradient 2) to afford 4-fluoro-2-[6-(3-{[1-(fluoromethyl)cyclopropyl]amino}pyrrolidin-1-yl)pyridazin-3-yl]-5-(6-methoxypyridazin-4-yl)phenol (60 mg, 55%) as a solid. LCMS (ES, m/z): 455 [M+H]

### Synthesis of Compounds 167 and 168

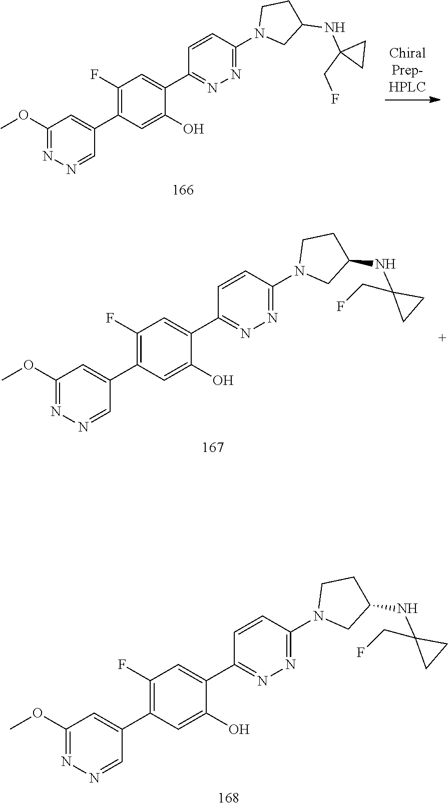 [SMILES: COc1cc(-c2cc(O)c(-c3ccc(N4CCC(NC5(CF)CC5)C4)nn3)cc2F)cnn1 ; "166" ; COc1cc(-c2cc(O)c(-c3ccc(N4CC[C@@H](NC5(CF)CC5)C4)nn3)cc2F)cnn1 ; "167" ; COc1cc(-c2cc(O)c(-c3ccc(N4CC[C@H](NC5(CF)CC5)C4)nn3)cc2F)cnn1 ; "168"]

The residue was purified by Prep-Chiral-HPLC with the following conditions: Column: CHIRAL ART Cellulose-SB, 2×25 cm, 5 m; Mobile Phase A: HEX: DCM=3: 1(0.1% DEA)-HPLC, Mobile Phase B: MeOH-HPLC; Flow rate: 20 mL/min; Gradient: 10% B to 10% B in 21 min; Wave Length: UV 254/220 nm; RT₁(min): 17.51; RT₂(min): 20.00; Sample Solvent: DCM-HPLC; Injection Volume: 0.4 mL; Number Of Runs: 5 to afford first eluting 4-fluoro-2-{6-[(3R)-3-{[1-(fluoromethyl)cyclopropyl]amino}pyrrolidin-1-yl]pyridazin-3-yl}-5-(6-methoxypyridazin-4-yl)phenol (7.0 mg, 12%) as a solid and second eluting 4-fluoro-2-{6-[(3S)-3-{[1-(fluoromethyl)cyclopropyl]amino}pyrrolidin-1-yl]pyridazin-3-yl}-5-(6-methoxypyridazin-4-yl)phenol (7.9 mg, 13%) as a solid. LCMS (ES, m/z): 455 [M+H]+H NMR (400 MHz, DMSO-d₆) δ 13.82 (s, 1H), 9.17 (t, J=1.9 Hz, 1H), 8.31 (d, J=9.9 Hz, 1H), 8.00 (d, J=12.5 Hz, 1H), 7.45 (dd, J=1.9, 1.0 Hz, 1H), 7.30 (d, J=6.9 Hz, 1H), 7.19 (d, J=9.7 Hz, 1H), 4.46 (s, 1H), 4.34 (s, 1H), 4.09 (s, 3H), 3.72 (s, 2H), 3.61 (s, 1H), 3.50 (s, 1H), 2.75 (s, 1H), 2.13 (d, J=12.8 Hz, 1H), 1.95-1.87 (m, 1H), 0.62 (s, 4H). LCMS (ES, m/z): 455 [M+H] ⁺¹H NMR (400 MHz, DMSO-d₆) δ 13.82 (s, 1H), 9.16 (t, J=1.9 Hz, 1H), 8.32 (d, J=9.9 Hz, 1H), 8.01 (d, J=12.5 Hz, 1H), 7.44 (dd, J=1.9, 1.0 Hz, 1H), 7.28 (d, J=6.9 Hz, 1H), 7.19 (d, J=9.7 Hz, 1H), 4.46 (s, 1H), 4.34 (s, 1H), 4.09 (s, 3H), 3.71 (s, 2H), 3.60 (s, 1H), 3.50 (s, 1H), 2.75 (s, 1H), 2.13 (d, J=12.8 Hz, 1H), 1.92-1.89 (m, 1H), 0.61 (s, 4H).

### Example 63: Synthesis of Compound 187

### Synthesis of Intermediate B167

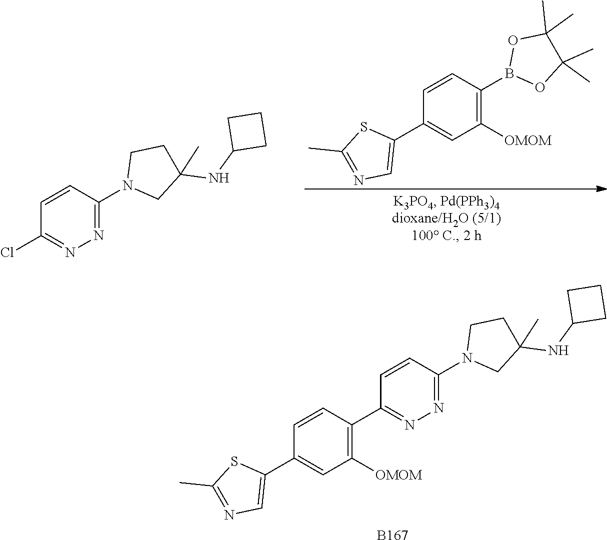

To a solution of 1-(6-chloropyridazin-3-yl)-N-cyclobutyl-3-methylpyrrolidin-3-amine (400 mg, 1.499 mmol, 1.0 equiv) and 5-[3-(methoxymethoxy)-4-(4,4,5,5-tetramethyl-1,3,2-dioxaborolan-2-yl) phenyl]-2-methyl-1,3-thiazole (595.85 mg, 1.649 mmol, 1.1 equiv) in dioxane (5 mL) and H₂O (1 mL) were added K₃PO₄ (795.69 mg, 3.748 mmol, 2.5 equiv) and Pd(PPh₃)₄(173.27 mg, 0.150 mmol, 0.1 equiv). After stirring for 2 h at 100° C. under a nitrogen atmosphere. The resulting mixture was extracted with EtOAc (3×50 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EA (1:4) to afford N-cyclobutyl-1-{6-[2-(methoxymethoxy)-4-(2-methyl-1,3-thiazol-5-yl) phenyl]pyridazin-3-yl}-3-methylpyrrolidin-3-amine (200 mg, 29%) as a solid.

### Synthesis of Compound 187

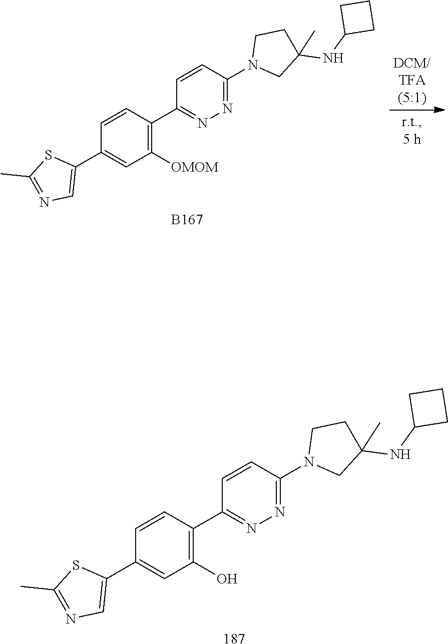

A solution of N-cyclobutyl-1-{6-[2-(methoxymethoxy)-4-(2-methyl-1,3-thiazol-5-yl) phenyl]pyridazin-3-yl}-3-methylpyrrolidin-3-amine (100 mg, 0.215 mmol, 1 equiv) in DCM (1 mL) was treated with TFA (200 uL) for 5 h at room temperature. The mixture was basified to pH 8 with saturated Na₂CO₃ (aq.). The resulting mixture was extracted with EtOAc (3×20 mL). The combined organic layers were washed with brine (3×10 mL), dried over anhydrous Na₂SO₄.

After filtration, the filtrate was concentrated under reduced pressure. The resulting. The crude product was purified by Prep-HPLC (Condition 6, Gradient 2) to afford 2-{6-[3-(cyclobutylamino)-3-methylpyrrolidin-1-yl]pyridazin-3-yl}-5-(2-methyl-1,3-thiazol-5-yl) phenol (7.5 mg, 8%) as a solid. LCMS: (ES, m/z): 421 [M+H] ⁺¹H NMR: (400 MHz, DMSO-d₆) δ14.02 (s, 1H), 8.21 (d, J=9.7 Hz, 1H), 8.10 (s, 1H), 7.90 (d, J=8.7 Hz, 1H), 7.21-7.07 (m, 3H), 3.61 (d, J=7.9 Hz, 1H), 3.57-3.48 (m, 1H), 3.42 (d, J=10.5 Hz, 1H), 3.34 (d, J=7.9 Hz, 1H), 3.29 (s, 1H), 2.68 (s, 3H), 2.12 (dtd, J=10.1, 7.0, 6.4, 3.0 Hz, 3H), 1.97 (dt, J=13.2, 7.2 Hz, 1H), 1.80 (dt, J=12.3, 7.3 Hz, 1H), 1.69 (dddd, J=19.5, 17.6, 9.7, 5.3 Hz, 2H), 1.59-1.47 (m, 2H), 1.21 (s, 3H).

### Example 64: Synthesis of Compounds 190, 191, and 192

### Synthesis of Intermediate B168

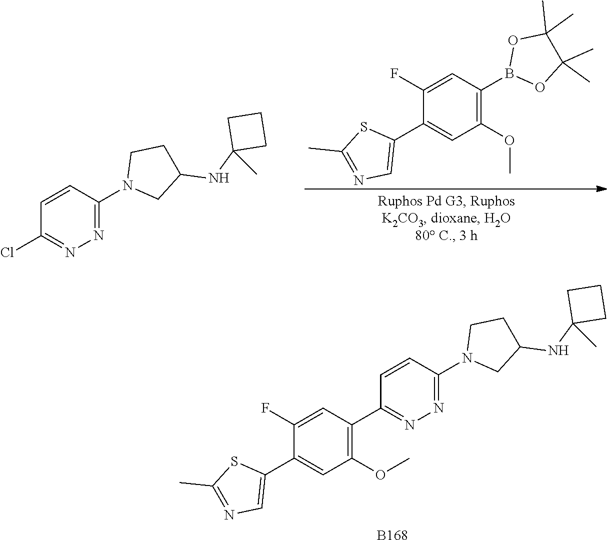

A solution of 1-(6-chloropyridazin-3-yl)-N-(1-methylcyclobutyl)pyrrolidin-3-amine (200 mg, 0.750 mmol, 1 equiv), 5-[2-fluoro-5-methoxy-4-(4,4,5,5-tetramethyl-1,3,2-dioxaborolan-2-yl)phenyl]-2-methyl-1,3-thiazole (288.0 mg, 0.825 mmol, 1.1 equiv), RuPhos Palladacycle Gen.3 (62.7 mg, 0.075 mmol, 0.1 equiv), RuPhos (34.98 mg, 0.075 mmol, 0.1 equiv) and K₂CO₃ (207.23 mg, 1.500 mmol, 2 equiv) in dioxane (5 mL) and H₂O (1 mL) was stirred for 3 h at 80° C. under nitrogen atmosphere. The resulting mixture was extracted with EtOAc (2×30 mL) and H₂O (30 mL). The combined organic layers were dried over anhydrous Na₂SO₄. After filtration, the filtrate was concentrated under reduced pressure. The residue was purified by silica gel column chromatography, eluted with PE/EA (1:5) to afford 1-{6-[5-fluoro-2-methoxy-4-(2-methyl-1,3-thiazol-5-yl)phenyl]pyridazin-3-yl}-N-(1-methylcyclobutyl)pyrrolidin-3-amine (200 mg, 59%) as a solid. LCMS:(ES, m/z):453

### Synthesis of Compound 190

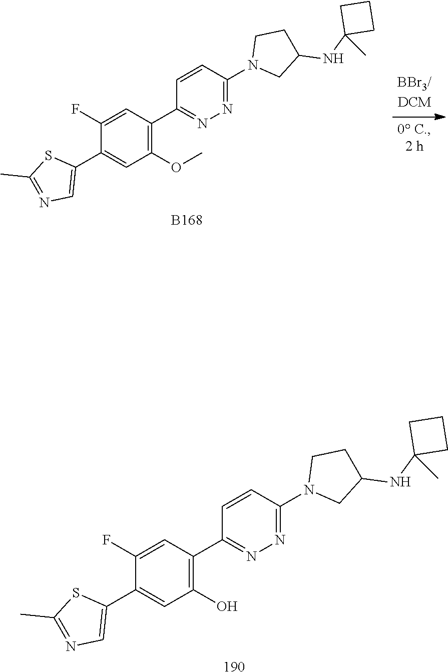 [SMILES: "B168" ; COc1cc(-c2cnc(C)s2)c(F)cc1-c1ccc(N2CCC(NC3(C)CCC3)C2)nn1 ; Cc1ncc(-c2cc(O)c(-c3ccc(N4CCC(NC5(C)CCC5)C4)nn3)cc2F)s1 ; "190"]

To a solution of 4-fluoro-5-(2-methyl-1,3-thiazol-5-yl)-2-(6-{3-[(1-methylcyclobutyl)amino]pyrrolidin-1-yl}pyridazin-3-yl)phenol (200 mg, 0.455 mmol, 1 equiv) in DCM (5 mL) was added BBr₃ (10 equiv) at 0° C. The mixture was stirred at room temperature for 2h. To MeOH (10 mL) was added the reaction mixture at 0° C. in dropwise. Then the mixture was concentrated and the crude product was purified by Prep-HPLC with the following conditions (2 #SHIMADZU (HPLC-01)): Column, Kinetex EVO C18 Column, 30*150.5 um; mobile phase, Water (10 mmol/L NH₄HCO₃) and ACN (20% ACN up to 60% in 10 min) to give 4-fluoro-5-(2-methyl-1,3-thiazol-5-yl)-2-(6-{3-[(1-methylcyclobutyl)amino]pyrrolidin-1-yl}pyridazin-3-yl)phenol (100 mg, 45%) as a solid. LCMS:(ES, m/z): 439.9[M+H]⁺

### Synthesis of Compounds 191 and 192

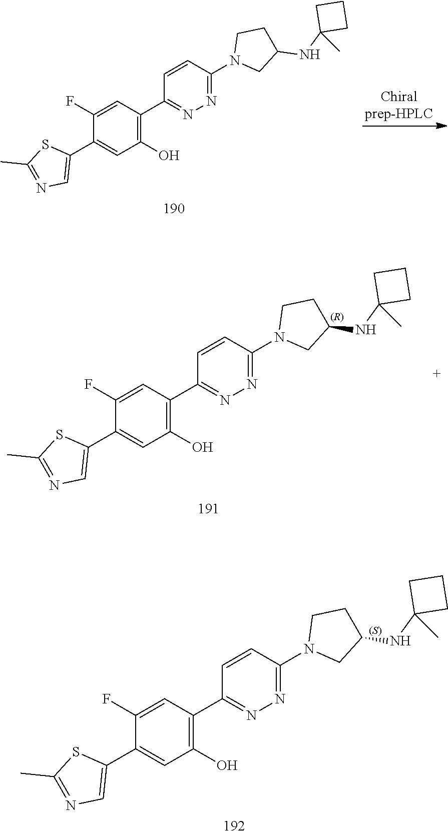

4-fluoro-5-(2-methyl-1,3-thiazol-5-yl)-2-(6-{3-[(1-methylcyclobutyl)amino]pyrrolidin-1-yl}pyridazin-3-yl)phenol (100 mg, 0.228 mmol, 1 equiv) was separated by Chiral prep-HPLC (Condition 11, Gradient 2) to afford 4-fluoro-5-(2-methyl-1,3-thiazol-5-yl)-2-{6-[(3R)-3-[(1-methylcyclobutyl)amino]pyrrolidin-1-yl]pyridazin-3-yl}phenol (12.6 mg, 12.60%) and 4-fluoro-5-(2-methyl-1,3-thiazol-5-yl)-2-{6-[(3R)-3-[(1-methylcyclobutyl)amino]pyrrolidin-1-yl]pyridazin-3-yl}phenol (11.8 mg, 12%) Compound 191: LCMS:(ES, m/z): 439.9[M+H]^{+ 1}H NMR: (400 MHz, DMSO-d₆) δ 13.73 (s, 1H), 8.25 (d, J=10.0 Hz, 1H), 8.16 (s, 1H), 7.93 (d, J=12.8 Hz, 1H), 7.29 (d, J=6.8 Hz, 1H), 7.17 (d, J=9.6 Hz, 1H), 3.76 (m, 1H), 3.74 (m, 1H), 3.48-3.46 (m, 2H), 3.18 (m, 1H), 2.70 (s, 3H), 2.16-2.14 (m, 1H), 1.95-1.90 (m, 2H), 1.81-1.76 (m, 3H), 1.69-1.66 (m, 2H), 1.26 (s, 3H). Compound 192: LCMS:(ES, m/z):439.9[M+H]^{+ 1}H NMR: (400 MHz, DMSO-d₆) δ 13.73 (s, 1H), 8.25 (d, J=10.0 Hz, 1H), 8.16 (s, 1H), 7.93 (d, J=12.8 Hz, 1H), 7.29 (d, J=6.8 Hz, 1H), 7.17 (d, J=9.6 Hz, 1H), 3.48 (m, 1H), 3.46 (m, 1H), 3.44-3.32 (m, 2H), 3.29 (m, 1H), 2.70 (s, 3H), 2.16-2.14 (m, 1H), 1.95-1.90 (m, 2H), 1.81-1.76 (m, 3H), 1.69-1.66 (m, 2H), 1.26 (s, 3H).

### Example 65: Synthesis of Compounds 205, 206, and 207

### Synthesis of Intermediate B169

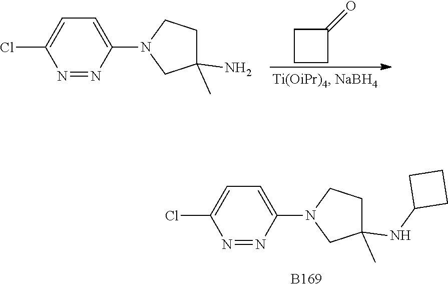 [SMILES: "B169" ; CC1(N)CCN(c2ccc(Cl)nn2)C1 ; CC1(NC2CCC2)CCN(c2ccc(Cl)nn2)C1 ; O=C1CCC1]

To a stirred mixture of (3S)-1-(6-chloropyridazin-3-yl)-3-methylpyrrolidin-3-amine (1 g, 4.702 mmol, 1 equiv) and cyclobutanone (1.32 g, 18.808 mmol, 4 equiv) in DCE (5 mL) were added NaBH₄ (0.89 g, 23.510 mmol, 5 equiv) in portions at 0° C. under air atmosphere. The reaction was quenched by the addition of Water (50 mL) at 0° C. The aqueous layer was extracted with EtOAc (2×50 mL), dried over anhydrous Na₂SO₄. After filtration, the filtrate was concentrated under reduced pressure. The residue was purified by silica gel column chromatography, eluted with CH₂Cl₂/MeOH (5:1) to afford (3S)-1-(6-chloropyridazin-3-yl)-N-cyclobutyl-3-methylpyrrolidin-3-amine (590 mg, 47%) as a solid. LCMS:(ES, m/z):266.90[M+H]⁺

### Synthesis of Intermediate B]70

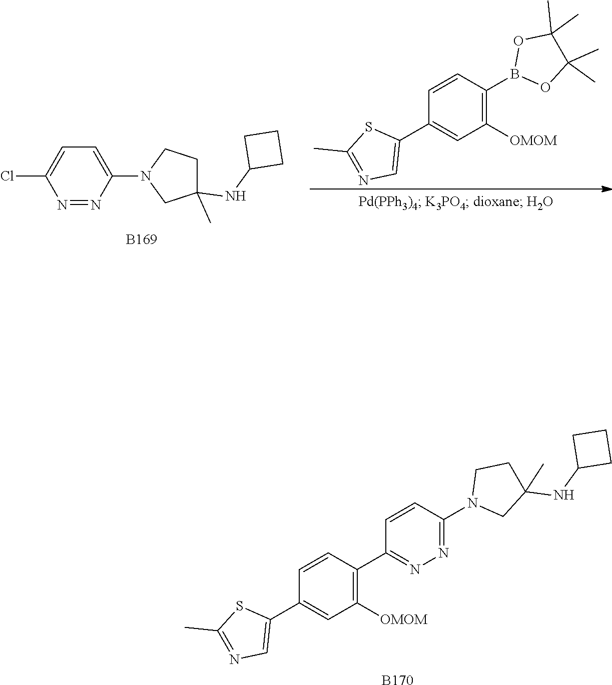

To a solution of 5-[3-(methoxymethoxy)-4-(4,4,5,5-tetramethyl-1,3,2-dioxaborolan-2-yl)phenyl]-2-methyl-1,3-thiazole (200 mg, 0.554 mmol, 1 equiv) and (3S)-1-(6-chloropyridazin-3-yl)-N-cyclobutyl-3-methylpyrrolidin-3-amine (103.38 mg, 0.388 mmol, 0.7 equiv) in dioxane (5 mL) and water (1 mL) were added K₃PO₄ (352.54 mg, 1.662 mmol, 3 equiv) and Pd(PPh₃)4 (63.98 mg, 0.055 mmol, 0.1 equiv). After stirring for 4 h at 80° C. under a nitrogen atmosphere, the resulting mixture was concentrated under reduced pressure. The residue was purified by silica gel column chromatography, eluted with CH₂Cl₂/MeOH (5:1) to afford (3S)—N-cyclobutyl-1-{6-[2-(methoxymethoxy)-4-(2-methyl-1,3-thiazol-5-yl)phenyl]pyridazin-3-yl}-3-methylpyrrolidin-3-amine (100 mg, 39%) as a solid. LCMS:(ES, m/z):466.05[M+H]⁺

### Synthesis of Compounds 205, 206, and 207

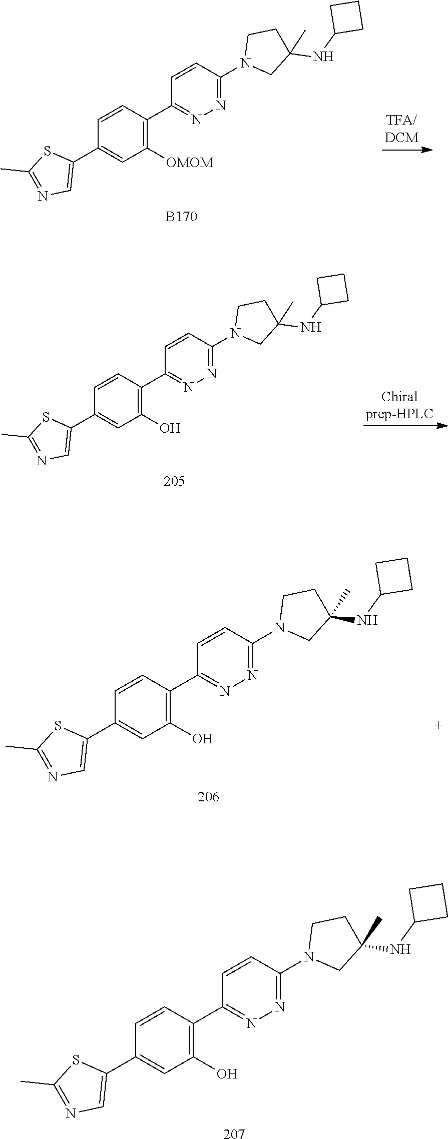

To a stirred solution of (3)-N-cyclobutyl-1-{6-[2-(methoxymethoxy)-4-(2-methyl-1,3-thiazol-5-yl)phenyl]pyridazin-3-yl}-3-methylpyrrolidin-3-amine (100 mg, 0.215 mmol, 1 equiv) in DCM(2 mL) was added TFA (1 mL) dropwise at 0° C. The crude product (50 mg) was purified by Prep-Chiral-HPLC (Condition 11, Gradient 2) to afford 2-{6-[(3)-3-(cyclobutylamino)-3-methylpyrrolidin-1-yl]pyridazin-3-yl}-5-(2-methyl-1,3-thiazol-5-yl)phenol (5.8 mg, 6.41%) as a solid and 2-{6-[(3R)-3-(cyclobutylamino)-3-methylpyrrolidin-1-yl]pyridazin-3-yl}-5-(2-methyl-1,3-thiazol-5-yl)phenol (2.2 mg, 4.38%) as a solid. Compound 206: LCMS:(ES, m/z):421.90[M+H]^{+ 1}H NMR:(400 MHz, DMSO-d₆) δ 14.02 (s, 1H), 9.06 (s, 2H), 8.21 (d, J=9.9 Hz, 1H), 8.10 (s, 1H), 7.91 (d, J=8.8 Hz, 1H), 7.20-7.10 (m, 3H), 3.98 (s, 1H), 3.74 (s, 1H), 3.62 (s, 1H), 3.54 (s, 2H), 2.68 (s, 3H), 2.18-2.07 (m, 6H), 1.86-1.62 (m, 2H), 1.58 (s, 3H).Compound 207: LCMS:(ES, m/z):421.90[M+H]^{+ 1}HNMR:(400 MHz, DMSO-d₆) δ 14.02 (s, 1H), 9.05 (s, 2H), 8.21 (d, J=9.9 Hz, 1H), 8.10 (s, 1H), 7.91 (d, J=8.8 Hz, 1H), 7.20-7.10 (m, 3H), 3.98 (s, 1H), 3.74 (s, 1H), 3.62 (s, 1H), 3.54 (s, 2H), 2.68 (s, 3H), 2.18-2.07 (m, 6H), 1.84-1.62 (m, 2H), 1.56 (s, 3H).

### Example 66: Synthesis of Compound 234

### Synthesis of Intermediate B]71

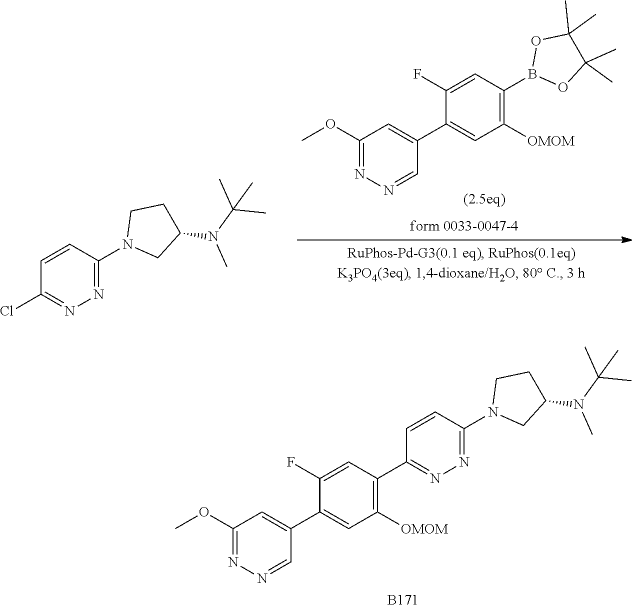

A solution of (3S)—N-tert-butyl-1-(6-chloropyridazin-3-yl)-N-methylpyrrolidin-3-amine (80 mg, 0.298 mmol, 1.00 equiv) in 1,4-dioxane (5 mL) and H₂O (1 mL) was treated with K₃PO₄ (189.53 mg, 0.894 mmol, 3 equiv) and RuPhos Palladacycle Gen.3 (24.89 mg, 0.030 mmol, 0.1 equiv) and RuPhos (13.89 mg, 0.030 mmol, 0.1 equiv) for 3 h at 80 C under nitrogen atmosphere followed by the addition of 5-[2-fluoro-5-(methoxymethoxy)-4-(4,4,5,5-tetramethyl-1,3,2-dioxaborolan-2-yl)phenyl]-3-methoxypyridazine (290.35 mg, 0.745 mmol, 2.5 equiv) dropwise portions at 80 degrees C. The mixture was allowed to cool down to room temperature. The reaction was quenched with H₂O at room temperature. The resulting mixture was extracted with EA (3×10 mL). The combined organic layers were washed with brine (2×10 mL) and dried over anhydrous Na₂SO₄. After filtration, the filtrate was concentrated under reduced pressure. The residue was purified by silica gel column chromatography, eluted with DCM:MeOH (9:1) to afford (3S)—N-tert-butyl-1-{6-[5-fluoro-2-(methoxymethoxy)-4-(6-methoxypyridazin-4-yl)phenyl]pyridazin-3-yl}-N-methylpyrrolidin-3-amine (76 mg, 51%) as a solid. LCMS (ES, m/z): 497 [M+H]⁺

### Synthesis of Compound 234

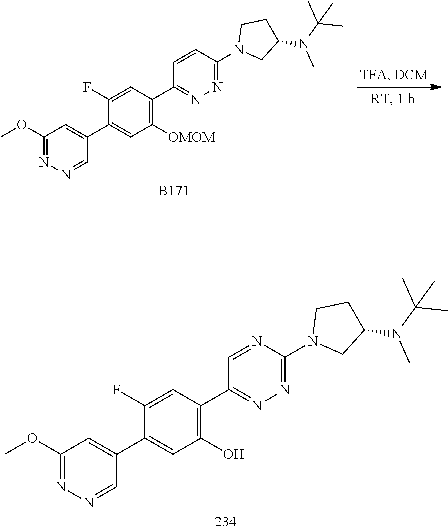

To a stirred solution of (3S)—N-tert-butyl-1-{6-[5-fluoro-2-(methoxymethoxy)-4-(6-methoxypyridazin-4-yl)phenyl]pyridazin-3-yl}-N-methylpyrrolidin-3-amine (76 mg, 0.153 mmol, 1 equiv) in DCM (2 mL) was added TFA (3 mL) dropwise at room temperature. The mixture was stirred 1 h at room temperature. The reaction liquid was concentrated under reduced pressure. The crude product was purified by reverse phase flash (Condition 8, Gradient 1) to afford 2-{6-[(3S)-3-[tert-butyl(methyl)amino]pyrrolidin-1-yl]pyridazin-3-yl}-4-fluoro-5-(6-methoxypyridazin-4-yl)phenol (3.2 mg, 4.62%) as a solid. LCMS: (ES, m/z): 453 [M+H] ^{+ 1}H NMR: (400 MHz, DMSO-d₆) δ 13.83 (s, 1H), 9.16 (d, J=1.9 Hz, 1H), 8.32 (d, J=9.8 Hz, 1H), 8.01 (d, J=12.4 Hz, 1H), 7.45 (d, J=1.7 Hz, 1H), 7.30 (d, J=6.9 Hz, 1H), 7.23 (d, J=9.8 Hz, 1H), 4.09 (s, 3H), 3.96 (p, J=8.2 Hz, 1H), 3.73 (s, 1H), 3.51 (s, 1H), 3.35 (s, 1H), 3.33 (s, 1H), 2.22 (s, 3H), 2.04 (q, J=10.2 Hz, 1H), 1.98-1.89 (m, 1H), 1.10 (s, 9H).

### Example 67: Synthesis of Compound 235

### Synthesis of Intermediate B172

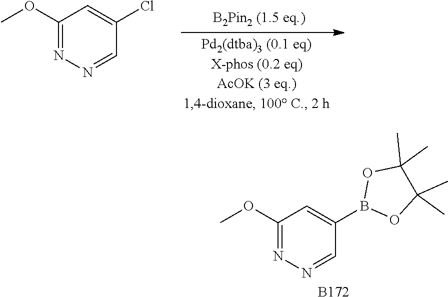

A solution of 5-chloro-3-methoxypyridazine (3 g, 20.753 mmol, 1 equiv) and 4,4,5,5-tetramethyl-2-(tetramethyl-1,3,2-dioxaborolan-2-yl)-1,3,2-dioxaborolane (7.90 g, 31.130 mmol, 1.5 equiv), AcOK (6.11 g, 62.259 mmol, 3 equiv) and Pd₂(dba)3 (1.90 g, 2.075 mmol, 0.1 equiv), X-Phos (1.98 g, 4.151 mmol, 0.2 equiv) in 1,4-dioxane(200 mL) was stirred for 2 h at 100° C. under nitrogen atmosphere. The resulting mixture was filtered and the filter cake was washed with 1,4-dioxane (3×30 mL). The filtrate was concentrated under reduced pressure. The crude product was used in the next step directly without further purification.

### Synthesis of Intermediate B173

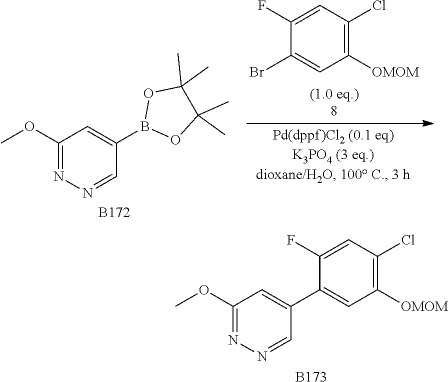

A solution of 3-methoxy-5-(4,4,5,5-tetramethyl-1,3,2-dioxaborolan-2-yl)pyridazine (3.8 g, 16.096 mmol, 1 equiv) and 1-bromo-4-chloro-2-fluoro-5-(methoxymethoxy)benzene (4.34 g, 16.096 mmol, 1 equiv) and Pd(dppf)Cl₂CH₂Cl₂ (1.31 g, 1.610 mmol, 0.1 equiv) and K₃PO₄ (10.25 g, 48.288 mmol, 3 equiv) in 1,4-dioxane/water=5:1 was stirred for 3 h at 100° C. under nitrogen atmosphere. The resulting mixture was filtered and the filter cake was washed with 1,4-dioxane (3×30 mL). The filtrate was concentrated under reduced pressure. The residue was purified by silica gel column chromatography, eluted with PE/EA (1:1) to afford 5-[4-chloro-2-fluoro-5-(methoxymethoxy)phenyl]-3-methoxypyridazine (1.2 g, 25%) as a solid.

### Synthesis of Intermediate B174

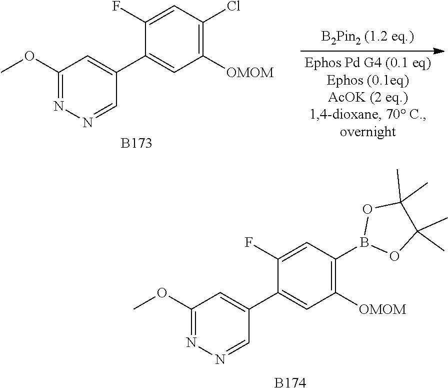

A solution of 5-[4-chloro-2-fluoro-5-(methoxymethoxy)phenyl]-3-methoxypyridazine (1.2 g, 4.017 mmol, 1 equiv) and 4,4,5,5-tetramethyl-2-(tetramethyl-1,3,2-dioxaborolan-2-yl)-1,3,2-dioxaborolane (1.22 g, 4.820 mmol, 1.2 equiv) and EPhos Pd G4 (0.37 g, 0.402 mmol, 0.1 equiv) and EPhos (0.21 g, 0.402 mmol, 0.1 equiv), AcOK (0.79 g, 8.034 mmol, 2 equiv) in 1,4-dioxane (15 mL) was stirred for overnight at 70° C. under nitrogen atmosphere. The resulting mixture was filtered and the filter cake was washed with 1,4-dioxane (3×30 mL). The filtrate was concentrated under reduced pressure. This resulted in 5-[2-fluoro-5-(methoxymethoxy)-4-(4,4,5,5-tetramethyl-1,3,2-dioxaborolan-2-yl)phenyl]-3-methoxypyridazine (1.5 g, 57%) as an oil.

### Synthesis of Intermediate B175

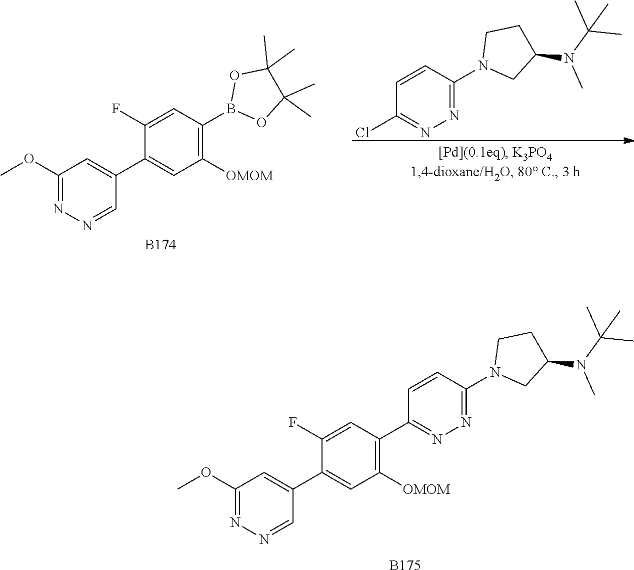

A solution 5-[2-fluoro-5-(methoxymethoxy)-4-(4,4,5,5-tetramethyl-1,3,2-dioxaborolan-2-yl) phenyl]-3-methoxypyridazine (400 mg, 1.025 mmol, 2.5 equiv) and (3R)—N-tert-butyl-1-(6-chloropyridazin-3-yl)-N-methylpyrrolidin-3-amine (110.21 mg, 0.410 mmol, 1 equiv) and RuPhos Palladacycle Gen.3 (34.29 mg, 0.041 mmol, 0.1 equiv) and RuPhos (19.13 mg, 0.041 mmol, 0.1 equiv), K₃PO₄ (261.1 mg, 1.230 mmol, 3 equiv) in 1,4-dioxane/H₂O=5:1(10 mL) was stirred for 3 h at 80° C. under nitrogen atmosphere. The resulting mixture was filtered and the filter cake was washed with 1,4-dioxane (3×30 mL). The filtrate was concentrated under reduced pressure. The residue was purified by silica gel column chromatography, eluted with CH₂Cl₂/MeOH (10:1) to afford (3R)—N-tert-butyl-1-{6-[5-fluoro-2-(methoxymethoxy)-4-(6-methoxypyridazin-4-yl)phenyl]pyridazin-3-yl}-N-methylpyrrolidin-3-amine (120 mg, 59%) as a solid.

### Synthesis of Compound 235

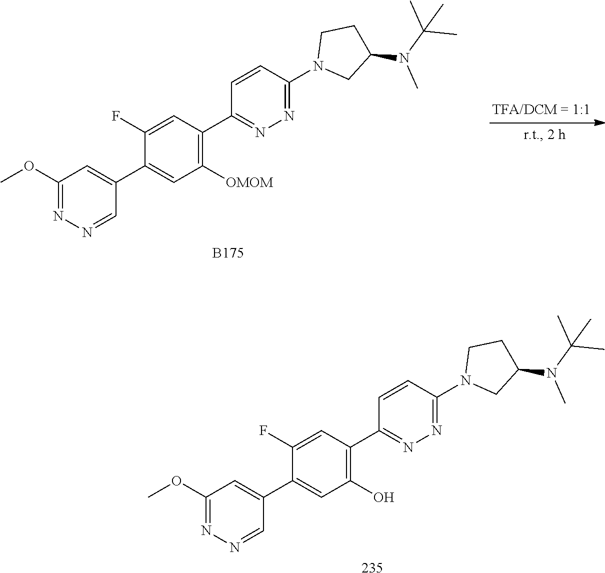

A solution of (3R)—N-tert-butyl-1-{6-[5-fluoro-2-(methoxymethoxy)-4-(6-methoxypyridazin-4-yl) phenyl]pyridazin-3-yl}-N-methylpyrrolidin-3-amine (120 mg, 0.242 mmol, 1 equiv) and trifluoroacetaldehyde (236.87 mg, 2.420 mmol, 10 equiv) in DCM(10 mL) was stirred for 2 h at room temperature under air atmosphere and purified by Prep-HPLC (Condition 6, Gradient 2) to afford 2-{6-[(3R)-3-[tert-butyl(methyl)amino]pyrrolidin-1-yl]pyridazin-3-yl}-4-fluoro-5-(6-methoxypyridazin-4-yl)phenol (20 mg, 18%) as a solid. LCMS (ES, m/z): 453 [M+H]⁺:¹H NMR (400 MHz, DMSO-d₆) δ 13.82 (s, 1H), 9.16 (t, J=1.8 Hz, 1H), 8.31 (d, J=9.7 Hz, 1H), 8.01 (d, J=12.4 Hz, 1H), 7.47-7.42 (m, 1H), 7.30 (d, J=6.9 Hz, 1H), 7.23 (d, J=9.7 Hz, 1H), 4.09 (s, 3H), 3.96 (p, J=8.5 Hz, 1H), 3.72 (s, 1H), 3.51 (t, J=9.4 Hz, 1H), 3.51 (d, J=6.9 Hz, 1H), 3.36 (d, J=6.9 Hz, 1H), 2.22 (s, 3H), 2.05 (p, J=10.1 Hz, 1H), 1.93 (dq, J=12.9, 7.1, 6.4 Hz, 1H), 1.10 (s, 9H).

### Example 68: Synthesis of Compounds 236 and 237

### Synthesis of Intermediate B176

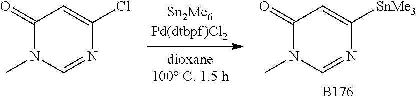

Into a 40 mL round-bottom flask were added 6-chloro-3-methylpyrimidin-4-one (500 mg, 3.459 mmol, 1 equiv), Pd(dtbpf)Cl₂ (225.43 mg, 0.346 mmol, 0.1 equiv) and dioxane (10 mL) at room temperature. The mixture was stirred for 1.5 h at 100° C. under nitrogen atmosphere. The reaction was quenched with KF(aq.) (20 mL) at room temperature. The aqueous layer was extracted with EtOAc (3×10 mL). The combined organic layers were dried over anhydrous Na₂SO₄. After filtration, the filtrate was concentrated under reduced pressure. The crude product 3-methyl-6-(trimethylstannyl)pyrimidin-4-one (1.3 g, 96%) as a solid was used in the next step directly without further purification. LCMS:(ES, m/z): 275[M+H]⁺

### Synthesis of Intermediate B177

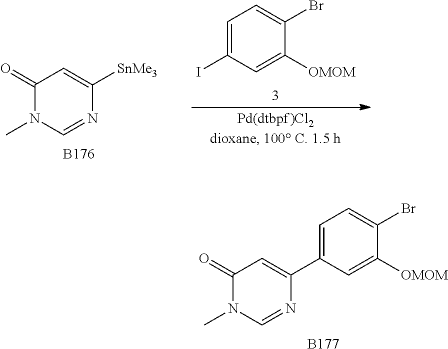

Into a 40 mL round-bottom flask were added 3-methyl-6-(trimethylstannyl)pyrimidin-4-one (1 g, 3.664 mmol, 1 equiv), 1-bromo-4-iodo-2-(methoxymethoxy)benzene (1.26 g, 3.664 mmol, 1 equiv), Pd(dtbpf)Cl₂ (0.24 g, 0.366 mmol, 0.1 equiv) and dioxane (15 mL, 177.059 mmol, 48.32 equiv) at room temperature. The resulting mixture was stirred for 1.5 h at 100° C. under nitrogen atmosphere. The resulting mixture was filtered and the filter cake was washed with CH₂Cl₂ (3×5 mL). The filtrate was concentrated under reduced pressure. The residue was purified by silica gel column chromatography, eluted with PE/EA (8:1) to afford 6-[4-bromo-3-(methoxymethoxy)phenyl]-3-methylpyrimidin-4-one (450 mg, 37.77%) as a solid. LCMS:(ES, m/z): 325[M+H]⁺

### Synthesis of Intermediate B]78

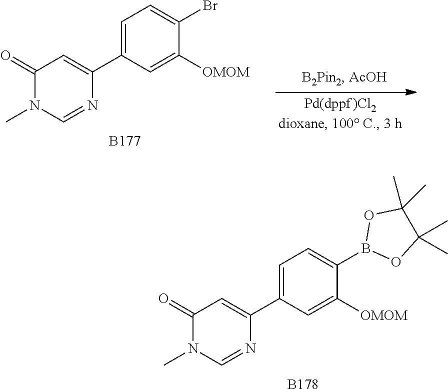

A solution of 6-[4-bromo-3-(methoxymethoxy)phenyl]-3-methylpyrimidin-4-one (450 mg, 1.384 mmol, 1 equiv) in dioxane (6 mL, 70.824 mmol, 51.18 equiv) was treated with Pd(dppf)Cl₂ (101.26 mg, 0.138 mmol, 0.1 equiv) and AcOK (135.82 mg, 1.384 mmol, 1 equiv) for 3 h at 100° C. under nitrogen atmosphere. The resulting mixture was filtered and the filter cake was washed with EtOAc (3×3 mL). The filtrate was concentrated under reduced pressure. The crude product (500 mg, 70%) was used in the next step directly without further purification. LCMS:(ES, m/z): 373[M+H]⁺

### Synthesis of Intermediate B]79

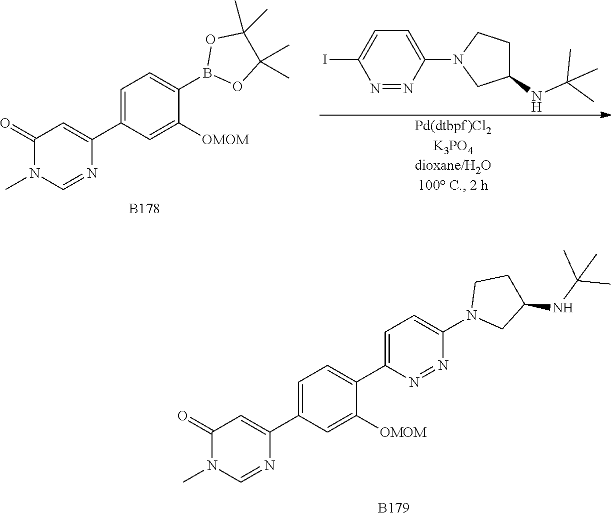

A solution of 6-[3-(methoxymethoxy)-4-(4,4,5,5-tetramethyl-1,3,2-dioxaborolan-2-yl)phenyl]-3-methylpyrimidin-4-one (200 mg, 0.537 mmol, 1 equiv) in dioxane (10 mL, 118.040 mmol, 219.69 equiv)/H₂O (2 mL, 111.019 mmol, 206.62 equiv) was treated with (3R)—N-tert-butyl-1-(6-iodopyridazin-3-yl)pyrrolidin-3-amine (186.02 mg, 0.537 mmol, 1 equiv), Pd(dtbpf)Cl₂ (43.77 mg, 0.054 mmol, 0.1 equiv) and K₃PO₄ (342.15 mg, 1.611 mmol, 3 equiv) for 2 h at 100° C. under nitrogen atmosphere. The resulting mixture was extracted with EtOAc (3×3 mL). The combined organic layers were dried over anhydrous Na₂SO₄. After filtration, the filtrate was concentrated under reduced pressure. The residue was purified by Prep-TLC (CH₂Cl₂/MeOH 10:1) to afford to afford 6-(4-{6-[(3R)-3-(tert-butylamino)pyrrolidin-1-yl]pyridazin-3-yl}-3-(methoxymethoxy)phenyl)-3-methylpyrimidin-4-one (30 mg, 12%) as a solid. LCMS:(ES, m/z): 465[M+H]⁺

### Synthesis of Intermediate B180

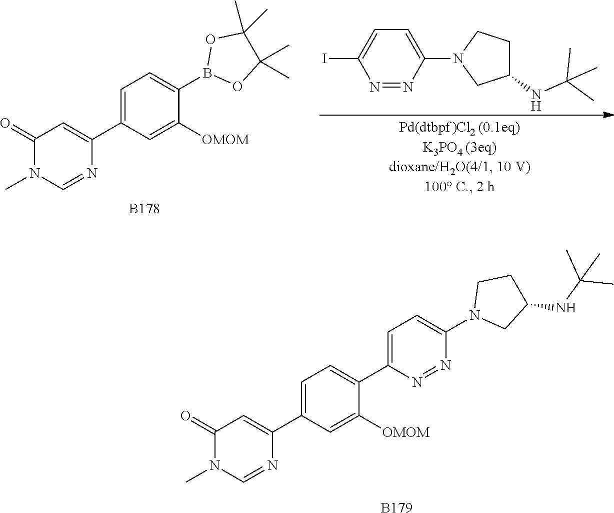

A solution of (3S)—N-tert-butyl-1-(6-iodopyridazin-3-yl)pyrrolidin-3-amine (90 mg, 0.260 mmol, 1 equiv) and 6-[3-(methoxymethoxy)-4-(4,4,5,5-tetramethyl-1,3,2-dioxaborolan-2-yl)phenyl]-3-methylpyrimidin-4-one (290 mg, 0.780 mmol, 3 equiv), Pd(DtBPF)Cl₂ (17 mg, 0.026 mmol, 0.1 equiv), K₃PO₄ (166 mg, 0.780 mmol, 3 equiv) in 1,4-dioxane (0.7 mL), water (0.2 mL) was stirred for 2 h at 100° C. under nitrogen atmosphere. The mixture was allowed to cool down to room temperature. The resulting mixture was extracted with EtOAc (1×mL). dried over anhydrous Na₂SO₄. After filtration, the filtrate was concentrated under reduced pressure. The residue was purified by silica gel column chromatography, eluted with CH₂Cl₂/MeOH (10:1) to afford 6-(4-{6-[(3S)-3-(tert-butylamino)pyrrolidin-1-yl]pyridazin-3-yl}-3-(methoxymethoxy)phenyl)-3-methylpyrimidin-4-one (26 mg, 22%) as a solid. LCMS:(ES, m/z): 465[M+H]⁺

### Synthesis of Compound 236

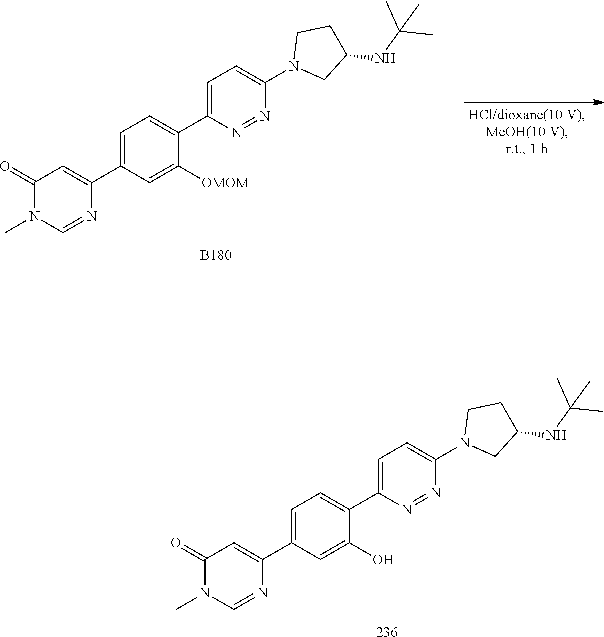

A solution of 6-(4-{6-[(3S)-3-(tert-butylamino)pyrrolidin-1-yl]pyridazin-3-yl}-3-(methoxymethoxy)phenyl)-3-methylpyrimidin-4-one (26 mg, 0.056 mmol, 1 equiv) in MeOH (0.25 mL), HCl(gas) in 1,4-dioxane (0.25 mL) was stirred for 1 h at room temperature. The resulting mixture was concentrated under reduced pressure. The crude product was purified by Prep-HPLC (Condition 6, Gradient 1) to afford 6-(4-{6-[(3S)-3-(tert-butylamino)pyrrolidin-1-yl]pyridazin-3-yl}-3-hydroxyphenyl)-3-methylpyrimidin-4-one (3.9 mg, 16%) as a solid.

LCMS:(ES, m/z):421[M+H]⁺¹H NMR: (400 MHz, DMSO-d₆) δ 13.87 (s, 1H), 8.57 (s, 1H), 8.27 (d, J=9.8 Hz, 1H), 7.98 (d, J=9.0 Hz, 1H), 7.65-7.58 (m, 2H), 7.17 (d, J=9.7 Hz, 1H), 7.00 (s, 1H), 3.80 (s, 1H), 3.65 (s, 1H), 3.54 (s, 1H), 3.44 (s, 4H), 3.09 (s, 1H), 2.18 (s, 1H), 1.77 (s, 2H), 1.09 (s, 9H).

### Synthesis of Compound 237

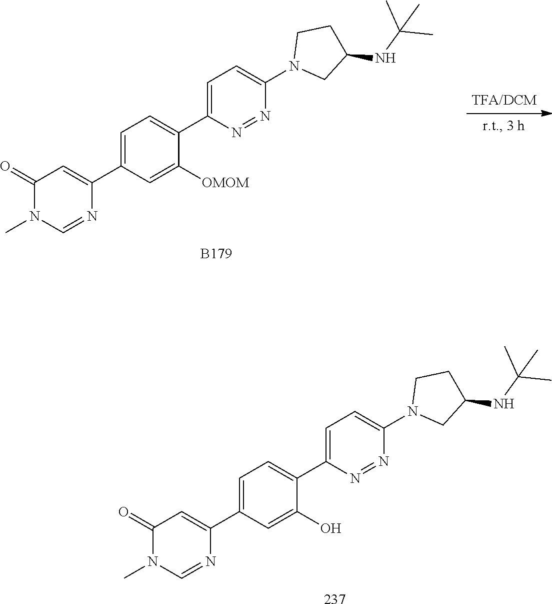

Into a 10 mL vial were added 6-(4-{6-[(3R)-3-(tert-butylamino)pyrrolidin-1-yl]pyridazin-3-yl}-3-(methoxymethoxy)phenyl)-3-methylpyrimidin-4-one (30 mg, 0.065 mmol, 1 equiv). TFA (1 mL, 13.463 mmol, 208.48 equiv) and DCM (3 mL, 47.192 mmol, 730.80 equiv). The solution was stirred for 3 h at room temperature. The crude product was purified by Chiral-Prep-HPLC (Condition 10, Gradient 3) to afford 6-(4-{6-[(3R)-3-(tert-butylamino)pyrrolidin-1-yl]pyridazin-3-yl}-3-hydroxyphenyl)-3-methylpyrimidin-4-one (3.1 mg, 11%) as a solid. LCMS:(ES, m/z):421[M+H]⁺¹H NMR: (400 MHz, DMSO-d₆) δ 13.88 (s, 1H), 8.57 (s, 1H), 8.27 (d, J=9.9 Hz, 1H), 7.98 (d, J=8.9 Hz, 1H), 7.61 (h, J=1.9 Hz, 2H), 7.16 (d, J=9.8 Hz, 1H), 7.00 (s, 1H), 3.79 (s, 1H), 3.65 (s, 1H), 3.54 (t, J=7.3 Hz, 1H), 6 3.44 (s, 4H), 3.09 (t, J=8.9 Hz, 1H), 2.18 (d, J=9.3 Hz, 1H), 1.85-1.70 (m, 2H), 1.09 (s, 9H).

### Example 69: Synthesis of Compounds 241 and 242

### Synthesis of Intermediate B181

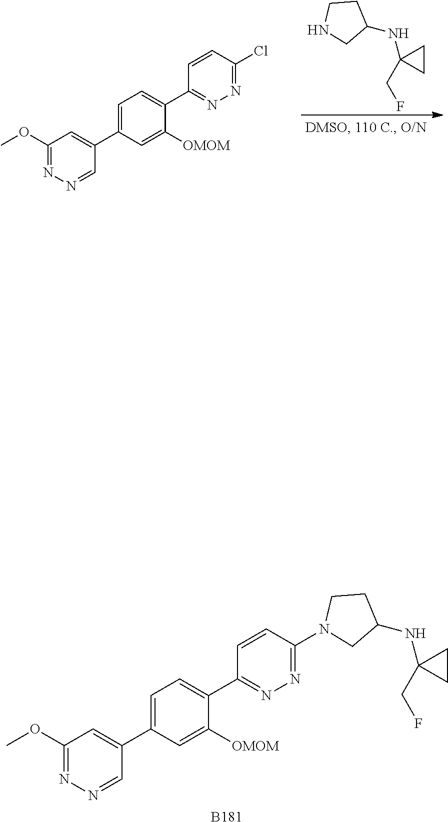

Into a 40 mL vial were added 5-[4-(6-chloropyridazin-3-yl)-3-(methoxymethoxy)phenyl]-3-methoxypyridazine (200 mg, 0.557 mmol, 1 equiv), N-[1-(fluoromethyl)cyclopropyl]pyrrolidin-3-amine (132.3 mg, 0.836 mmol, 1.5 equiv), CsF (254.03 mg, 1.671 mmol, 3 equiv) and DMSO (4 mL) at room temperature. The resulting mixture was stirred for overnight at 80° C. The resulting mixture was extracted with EtOAc (5×20 mL). The combined organic layers were dried over anhydrous Na₂SO₄. After filtration, the filtrate was concentrated under reduced pressure. The residue was purified by silica gel column chromatography, eluted with PE/EA (5:1) to afford N-[1-(fluoromethyl)cyclopropyl]-1-{6-[2-(methoxymethoxy)-4-(6-methoxypyridazin-4-yl)phenyl]pyridazin-3-yl}pyrrolidin-3-amine (70 mg, 26%) as a solid. LCMS (ESI, m/z): 481[M+H]⁺

### Synthesis of Intermediates B182 and B183

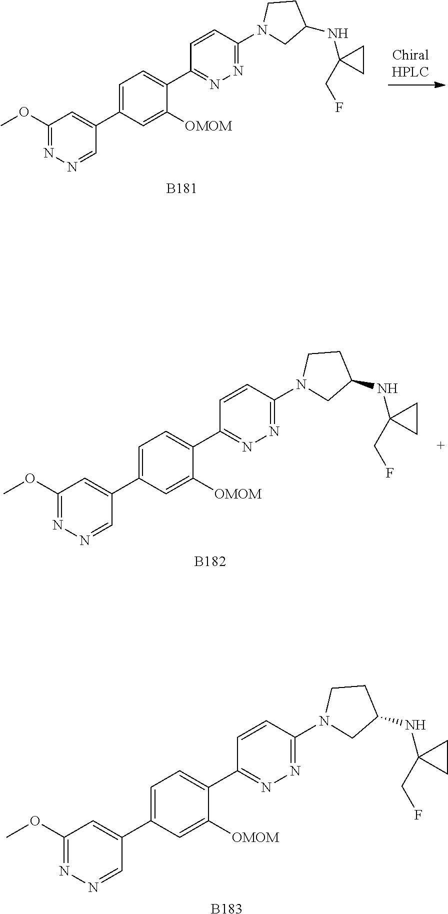

The residue was purified by Prep-Chiral-HPLC (Condition 11, Gradient 3) to afford (3R)—N-[1-(fluoromethyl)cyclopropyl]-1-{6-[2-(methoxymethoxy)-4-(6-methoxypyridazin-4-yl)phenyl]pyridazin-3-yl}pyrrolidin-3-amine (15 mg, 21.43%) as a white solid. This resulted in (3S)—N-[1-(fluoromethyl)cyclopropyl]-1-{6-[2-(methoxymethoxy)-4-(6-methoxypyridazin-4-yl)phenyl]pyridazin-3-yl}pyrrolidin-3-amine (20 mg, 29%) as a solid. LCMS (ESI, m/z): 481[M+H]⁺

### Synthesis of Compounds 241

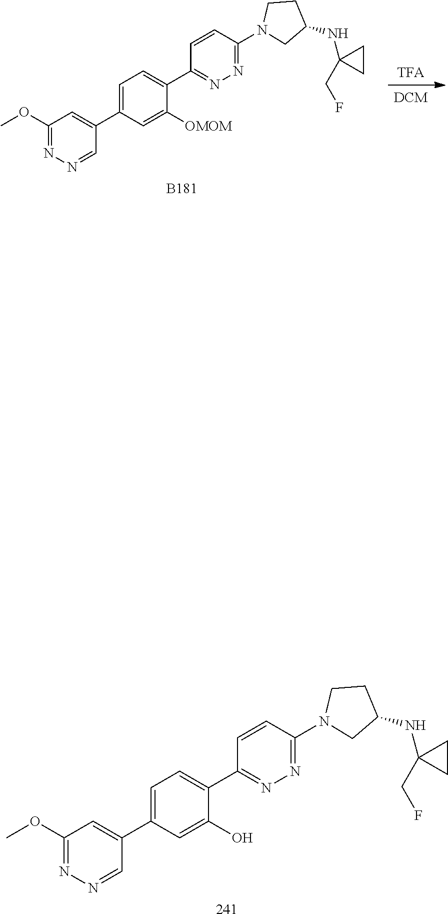

A solution of 2-[6-(3-{[1-(fluoromethyl)cyclopropyl]amino}pyrrolidin-1-yl)pyridazin-3-yl]-5-(6-methoxypyridazin-4-yl)phenol (20 mg, 0.046 mmol, 1 equiv) and TFA (1 mL, 13.463 mmol, 293.83 equiv) in DCM (1 mL) was stirred for 2 h at room temperature. The resulting mixture was concentrated under reduced pressure. The crude product was purified by Prep-HPLC (Condition 6, Gradient 1) to afford 2-{6-[(3S)-3-{[1-(fluoromethyl)cyclopropyl]amino}pyrrolidin-1-yl]pyridazin-3-yl}-5-(6-methoxypyridazin-4-yl)phenol (5.9 mg, 29%) as a solid. LCMS (ESI, m/z): 437[M+H]^{+ 1}H NMR (400 MHz, DMSO-d₆) δ 14.12 (s, 1H), 9.35 (d, J=1.9 Hz, 1H), 8.30 (d, J=9.8 Hz, 1H), 8.04 (d, J=8.3 Hz, 1H), 7.56 (d, J=1.9 Hz, 1H), 7.53-7.42 (m, 1H), 7.18 (d, J=9.8 Hz, 1H), 4.46 (s, 1H), 4.34 (s, 1H), 4.08 (s, 3H), 3.72 (d, J=7.1 Hz, 2H), 3.60 (s, 1H), 3.50 (d, J=9.0 Hz, 1H), 3.33 (s, 1H), 2.73 (s, 1H), 2.27-2.14 (m, 1H), 1.98-1.90 (m, 1H), 0.61 (d, J=4.8 Hz, 4H).

### Synthesis of Compounds 242

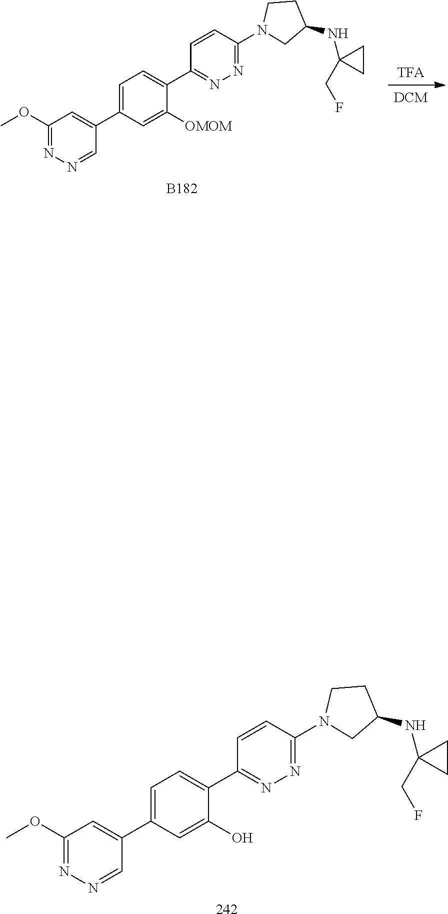

A solution of 2-[6-(3-{[1-(fluoromethyl)cyclopropyl]amino}pyrrolidin-1-yl)pyridazin-3-yl]-5-(6-methoxypyridazin-4-yl)phenol (15 mg, 0.034 mmol, 1 equiv) and TFA (1 mL, 13.463 mmol, 391.77 equiv) in DCM (1 mL) was stirred for 2 h at room temperature. The resulting mixture was concentrated under reduced pressure. The crude product was purified by Prep-HPLC (Condition 6, Gradient 1) to afford 2-{6-[(3R)-3-{[1-(fluoromethyl)cyclopropyl]amino}pyrrolidin-1-yl]pyridazin-3-yl}-5-(6-methoxypyridazin-4-yl)phenol (9.2 mg, 61%) as a solid. LCMS (ESI, m/z): 437[M+H]^{+ 1}H NMR (400 MHz, DMSO-d₆) δ 14.12 (s, 1H), 9.36 (d, J=1.9 Hz, 1H), 8.31 (d, J=9.9 Hz, 1H), 8.05 (d, J=8.4 Hz, 1H), 7.56 (d, J=1.9 Hz, 1H), 7.53-7.44 (m, 2H), 7.18 (d, J=9.8 Hz, 1H), 4.46 (s, 1H), 4.34 (s, 1H), 4.08 (s, 3H), 3.72 (d, J=6.8 Hz, 2H), 3.50 (s, 1H), 3.33 (s, 1H), 2.84-2.62 (m, 1H), 2.25-2.09 (m, 1H), 1.97-1.83 (m, 1H), 0.62 (s, 4H).

### Example 70: Synthesis of Compounds 265 and 266

### Synthesis of Intermediate B183

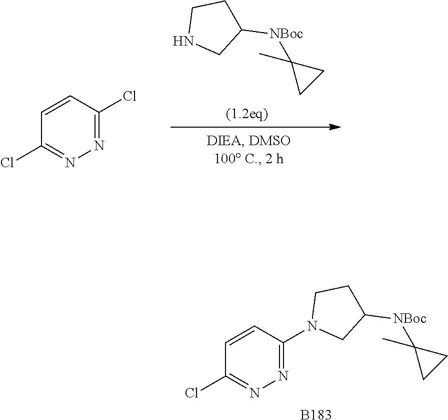

A solution of pyridazine, 3,6-dichloro- (1 g, 6.713 mmol, 1 equiv) and tert-butyl N-(1-methylcyclopropyl)-N-(pyrrolidin-3-yl)carbamate (1.94 g, 8.056 mmol, 1.2 equiv), DIEA (2.60 g, 20.139 mmol, 3 equiv) in DMSO (10 mL) was stirred for 2 h at 100° C. The mixture was allowed to cool down to room temperature. The resulting mixture was poured into water. The resulting mixture was filtered and the filter cake was washed with water (1×5 mL). The cake was concentrated under reduced pressure. This resulted in tert-butyl N-[1-(6-chloropyridazin-3-yl)pyrrolidin-3-yl]-N-(1-methylcyclopropyl)carbamate (2 g, 84%) as an solid. LCMS:(ES, m/z):353

### Synthesis of Intermediate B184

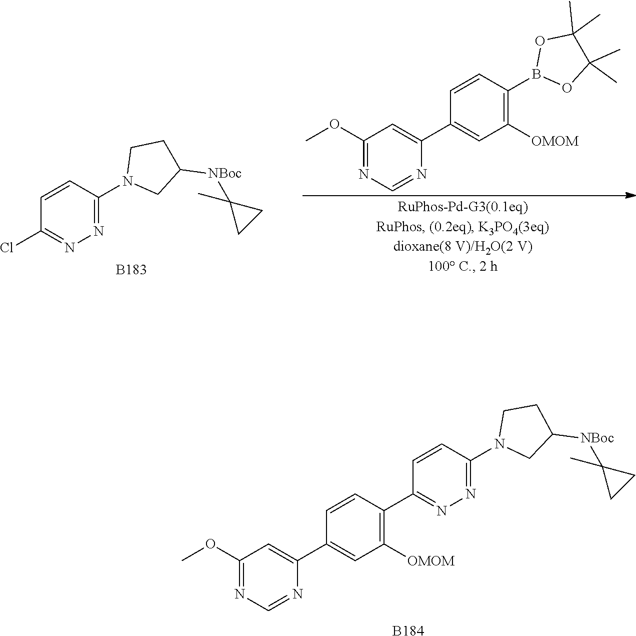

A solution of tert-butyl N-[1-(6-chloropyridazin-3-yl)pyrrolidin-3-yl]-N-(1-methylcyclopropyl)carbamate (200 mg, 0.567 mmol, 1 equiv) and 3-methoxy-5-[3-(methoxymethoxy)-4-(4,4,5,5-tetramethyl-1,3,2-dioxaborolan-2-yl)phenyl]pyridazine (422 mg, 1.134 mmol, 2 equiv), RuPhos Palladacycle Gen.3 (47 mg, 0.057 mmol, 0.1 equiv), RuPhos (53 mg, 0.113 mmol, 0.2 equiv),K₃PO₄ (361 mg, 1.701 mmol, 3 equiv) in 1,4-dioxane (1.6 mL), water (0.4 mL) was stirred for 2 h at 100° C. under nitrogen atmosphere. The mixture was allowed to cool down to room temperature. The resulting mixture was poured into water. The resulting mixture was extracted with EA (1×9 mL). dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-(1-{6-[2-(methoxymethoxy)-4-(6-methoxypyridazin-4-yl)phenyl]pyridazin-3-yl}pyrrolidin-3-yl)-N-(1-methylcyclopropyl)carbamate (300 mg, 94%) as a solid. LCMS:(ES, m/z):563

### Synthesis of Intermediates B185 and B186

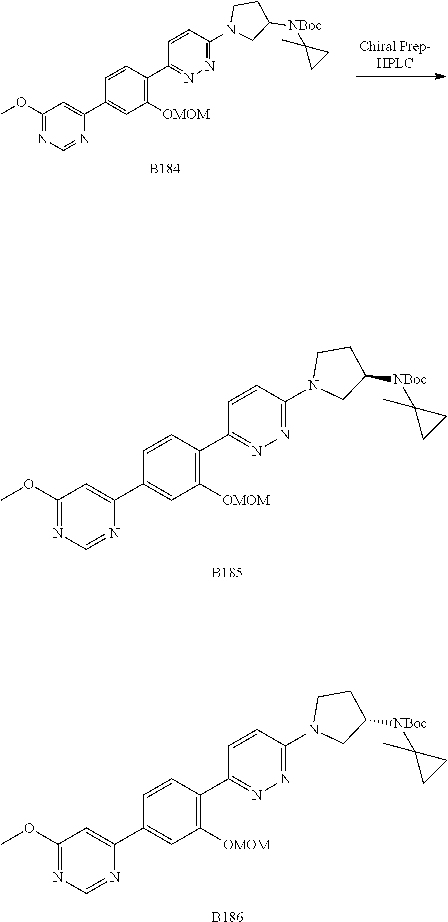

The crude product (150 mg) was purified by Prep-Chiral-HPLC (Condition 11, Gradient 2) to afford Intermediate B185 (59 mg) as a solid and Intermediate B186 (61 mg) as a solid.

### Synthesis of Compound 266

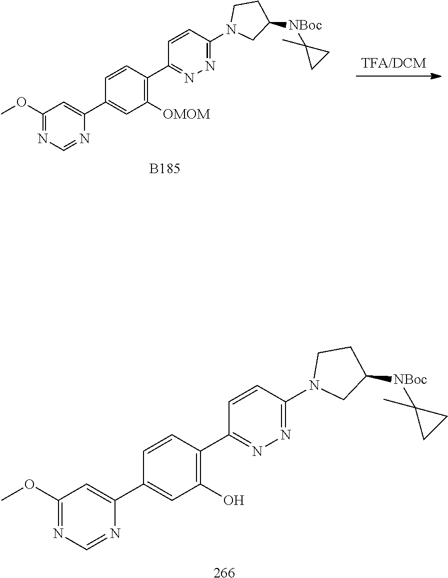

A solution of tert-butyl N-[(3R)-1-{6-[2-(methoxymethoxy)-4-(6-methoxypyridazin-4-yl)phenyl]pyridazin-3-yl}pyrrolidin-3-yl]-N-(1-methylcyclopropyl)carbamate (59 mg, 0.105 mmol, 1 equiv) in DCM (1.8 mL). TFA (0.6 mL) was stirred for 1 h at room temperature. The resulting mixture was concentrated under reduced pressure. The crude product was purified by Prep-HPLC (Condition 6, Gradient 1) to afford 5-(6-methoxypyridazin-4-yl)-2-{6-[(3R)-3-[(1-methylcyclopropyl)amino]pyrrolidin-1-yl]pyridazin-3-yl}phenol (18.4 mg, 41%) as a solid. LCMS:(ES, m/z):419 ¹HNMR: (400 MHz, DMSO-d₆) δ 14.11 (s, 1H), 9.35 (d, J=1.9 Hz, 1H), 8.29 (d, J=9.9 Hz, 1H), 8.04 (d, J=8.3 Hz, 1H), 7.55 (d, J=2.0 Hz, 1H), 7.52-7.44 (m, 2H), 7.17 (d, J=9.7 Hz, 1H), 4.09 (s, 3H), 3.76-3.66 (m, 1H), 3.64-3.60 (q, J=6.0 Hz, 2H), 3.49 (q, J=8.7, 8.1 Hz, 1H), 3.29 (s, 1H), 2.40 (s, 1H), 2.13 (dq, J=12.5, 6.2 Hz, 1H), 1.89 (dt, J 12.2, 6.9 Hz, 1H), 1.26 (s, 3H), 0.53-0.40 (m, 2H), 0.39-0.27 (m, 2H).

### Synthesis of Compound 265

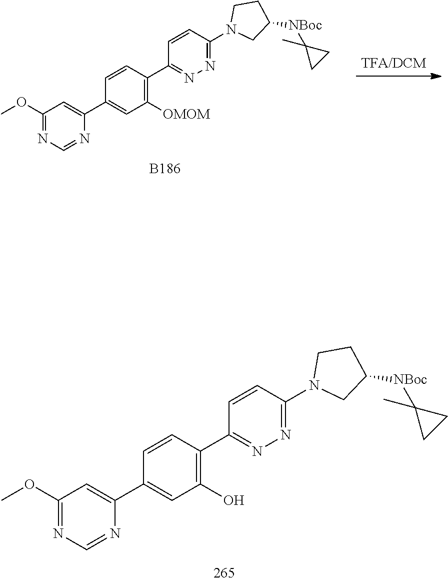

A solution of tert-butyl N-[(3S)-1-{6-[2-(methoxymethoxy)-4-(6-methoxypyridazin-4-yl)phenyl]pyridazin-3-yl}pyrrolidin-3-yl]-N-(1-methylcyclopropyl)carbamate (61 mg, 0.108 mmol, 1 equiv) in DCM (1.8 mL). TFA (0.6 mL) was stirred for 1 h at room temperature. The resulting mixture was concentrated under reduced pressure. The crude product was purified by Prep-HPLC (Condition 6, Gradient 1) to afford 5-(6-methoxypyridazin-4-yl)-2-{6-[(3S)-3-[(1-methylcyclopropyl)amino]pyrrolidin-1-yl]pyridazin-3-yl}phenol (19.2 mg, 42%) as a solid. LCMS:(ES, m/z):419 ¹HNMR: (400 MHz, DMSO-d₆) δ 14.11 (s, 1H), 9.35 (d, J=1.9 Hz, 1H), 8.29 (d, J=9.8 Hz, 1H), 8.04 (d, J=8.3 Hz, 1H), 7.55 (d, J=1.9 Hz, 1H), 7.52-7.44 (m, 2H), 7.17 (d, J=9.8 Hz, 1H), 4.09 (s, 3H), 3.72 (dd, J=10.4, 6.1 Hz, 1H), 3.65-3.60 (p, J=6.0 Hz, 2H), 3.50 (t, J=8.1 Hz, 1H), 3.29 (s, 1H), 2.40 (s, 1H), 2.13 (dq, J=12.3, 6.1 Hz, 1H), 1.88 (dq, J=13.8, 7.1 Hz, 1H), 1.26 (s, 3H), 0.52-0.40 (m, 2H), 0.33 (q, J=2.6, 2.0 Hz, 2H).

### Example 71: Synthesis of compound 268 from compound 148

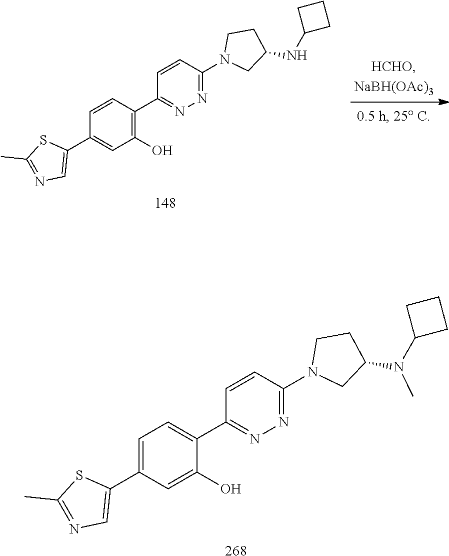 [SMILES: Cc1ncc(-c2ccc(-c3ccc(N4CC[C@H](NC5CCC5)C4)nn3)c(O)c2)s1 ; "148" ; Cc1ncc(-c2ccc(-c3ccc(N4CC[C@H](N(C)C5CCC5)C4)nn3)c(O)c2)s1 ; "268"]

To a solution of tert-butyl N-cyclobutyl-N-[(3S)-1-{6-[2-hydroxy-4-(2-methyl-1,3-thiazol-5-yl)phenyl]pyridazin-3-yl}pyrrolidin-3-yl]carbamate (100 mg, 0.197 mmol, 1 equiv) in DCE (2 mL) was added HCHO (29.57 mg, 0.985 mmol, 5 equiv), after stirring for 0.5 h at 25°, STAB (125.25 mg, 0.591 mmol, 3 equiv) was added to the mixture. And the reaction was stirred for 2 h at 25° under a nitrogen atmosphere. The reaction was quenched with water (0.5 mL) at 0° C. the resulting mixture was concentrated under reduced pressure. The crude product was purified by Chiral-Prep-HPLC with the following conditions (2 #SHIMADZU (HPLC-01)): Column, XBridge Shield RP18 OBD Column, 30*150 mm, 5 pm; mobile phase, Water (10 mmol/L NH₄HCO₃) and ACN (20% ACN up to 50% in 10 min) to afford 2-{6-[(3S)-3-(cyclobutylamino)pyrrolidin-1-yl]pyridazin-3-yl}-5-(2-methyl-1,3-thiazol-5-yl) phenol (23.3 mg, 29%) as an solid. LCMS (ES, m/z): 421.9 [M+H]^{+ 1}H NMR (400 MHz, DMSO-d₆) δ 8.39 (s, 1H), 8.16 (s, 1H), 7.82 (d, J=9.5 Hz, 1H), 7.72 (d, J=8.2 Hz, 1H), 7.67 (d, J=1.8 Hz, 1H), 7.44 (dd, J=8.2, 1.8 Hz, 1H), 6.94 (d, J=9.5 Hz, 1H), 3.69 (q, J=9.5, 8.7 Hz, 2H), 3.41 (dt, J 10.1, 5.2 Hz, 1H), 3.26 (dd, J=10.3, 8.2 Hz, 1H), 3.13 (t, J=8.0 Hz, 1H), 3.06-2.96 (m, 1H), 2.66 (s, 3H), 2.10 (s, 4H), 1.99 (tq, J=6.8, 4.0, 2.9 Hz, 2H), 1.88 (dtd, J=11.7, 9.4, 5.3 Hz, 3H), 1.59 (ddt, J=14.5, 6.4, 3.4 Hz, 2H).

### Example 72: Synthesis of Compound 269 (from Compound 146)

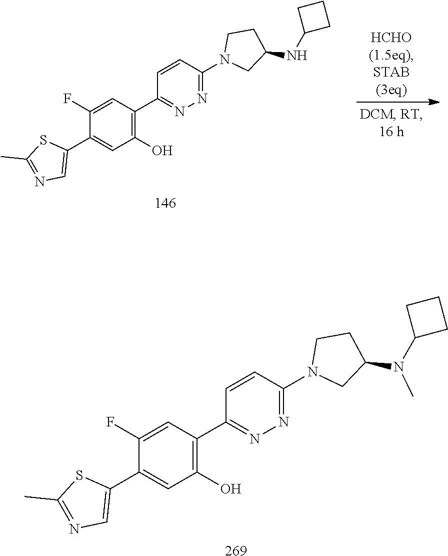 [SMILES: Cc1ncc(-c2cc(O)c(-c3ccc(N4CC[C@@H](NC5CCC5)C4)nn3)cc2F)s1 ; "146" ; Cc1ncc(-c2cc(O)c(-c3ccc(N4CC[C@@H](N(C)C5CCC5)C4)nn3)cc2F)s1 ; "269"]

A solution of 2-{6-[(3R)-3-(cyclobutylamino)pyrrolidin-1-yl]pyridazin-3-yl}-4-fluoro-5-(2-methyl-1,3-thiazol-5-yl)phenol (200 mg, 0.470 mmol, 1 equiv) in DCM (4 mL) was treated with HCHO (21.17 mg, 0.705 mmol, 1.5 equiv) for 2 h at room temperature under nitrogen atmosphere followed by the addition of STAB (298.84 mg, 1.410 mmol, 3 equiv) dropwise portions at 0 degrees C. The resulting mixture was stirred for additional 16 h at room temperature. The mixture was basified to pH 8 with NaOH Solution. The resulting mixture was diluted with H₂O (20 mL). The resulting mixture was extracted with DCM (2×50 mL). The combined organic layers were washed with NaCl Solution (1×20 mL), dried over anhydrous Na₂SO₄. After filtration, the filtrate was concentrated under reduced pressure. The crude product was purified by reverse phase flash (Condition 6, Gradient 2) to afford 2-{6-[(3R)-3-(cyclobutylamino)pyrrolidin-1-yl]pyridazin-3-yl}-4-fluoro-5-(2-methyl-1,3-thiazol-5-yl)phenol (24.6 mg, 12%) as a solid. LCMS: (ES, m/z): 440 [M+H]^{+ 1}H NMR (400 MHz, DMSO-d₆) δ 13.73 (s, 1H), 8.27 (d, J=9.8 Hz, 1H), 8.17 (s, 1H), 7.95 (d, J=12.6 Hz, 1H), 7.30 (d, J=6.8 Hz, 1H), 7.20 (d, J=9.7 Hz, 1H), 3.72 (d, J=11.3 Hz, 2H), 3.48-3.39 (m, 1H), 3.29 (dd, J=18.2, 9.3 Hz, 1H), 3.13 (t, J=8.1 Hz, 1H), 3.06-2.96 (m, 1H), 2.71 (s, 3H), 2.13 (dd, J=9.0, 3.9 Hz, 1H), 2.09 (s, 3H), 2.05-1.94 (m, 2H), 1.91 (dd, J=9.7, 2.8 Hz, 1H), 1.86 (d, J=10.3 Hz, 2H), 1.64-1.51 (m, 2H).

### Example 73: Synthesis of Compound 273

### Synthesis of Intermediate B187

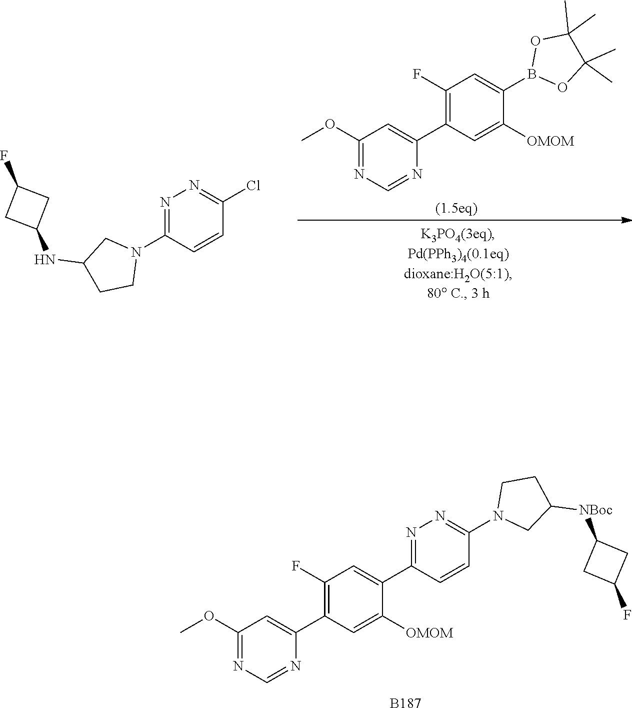

To a solution of 1-(6-chloropyridazin-3-yl)-N-[(1s,3s)-3-fluorocyclobutyl]pyrrolidin-3-amine (220 mg, 0.813 mmol, 1 equiv) and 5-[2-fluoro-5-(methoxymethoxy)-4-(4,4,5,5-tetramethyl-1,3,2-dioxaborolan-2-yl)phenyl]-3-methoxypyridazine (475.63 mg, 1.220 mmol, 1.5 equiv) in dioxane (4.5 mL) and H₂O (0.9 mL) were added K₃PO₄ (517.45 mg, 2.439 mmol, 3 equiv) and Pd(PPh₃)4 (21.31 mg, 0.081 mmol, 0.1 equiv). After stirring for 3 h at 80° C. under a nitrogen atmosphere, the resulting mixture was concentrated under reduced pressure. The residue was purified by Prep-silica gel column chromatography, eluted with PE/EA (3:1) to afford tert-butyl N-(1-{6-[5-fluoro-2-(methoxymethoxy)-4-(6-methoxypyridazin-4-yl)phenyl]pyridazin-3-yl}pyrrolidin-3-yl)-N-[(1s,3s)-3-fluorocyclobutyl]carbamate (170 mg, 35%) as a solid. LCMS: (ES, m/z):599 [M+H]⁺

### Synthesis of Compound 273

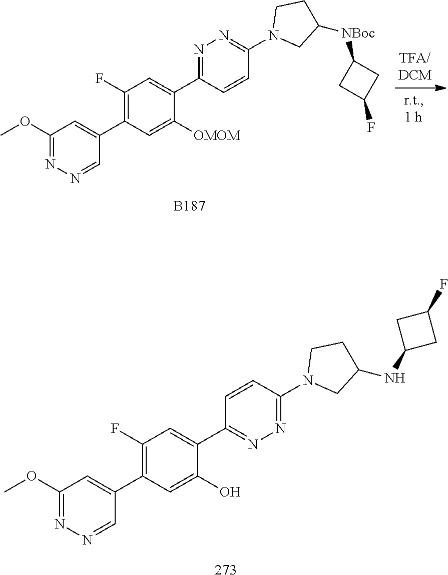

A solution of tert-butyl N-(1-{6-[5-fluoro-2-(methoxymethoxy)-4-(6-methoxypyridazin-4-yl)phenyl]pyridazin-3-yl}pyrrolidin-3-yl)-N-[(1s,3s)-3-fluorocyclobutyl]carbamate (150 mg, 0.251 mmol, 1 equiv) and TFA (4 mL, 53.852 mmol, 214.93 equiv) in DCM (4 mL) was stirred for 1 h at room temperature. The resulting mixture was concentrated under reduced pressure. The crude product (100 mg) was purified by Prep-HPLC (Condition 6, Gradient 1) to afford 4-fluoro-5-(6-methoxypyridazin-4-yl)-2-[6-(3-{[(1s,3s)-3-fluorocyclobutyl]amino}pyrrolidin-1-yl)pyridazin-3-yl]phenol (33.3 mg, 29%) as a solid. LCMS: (ES, m/z): 455 [M+H]H NMR: (400 MHz, DMSO-d₆) δ 13.82 (s, 1H), 9.17 (t, J=1.9 Hz, 1H), 8.32 (d, J=9.9 Hz, 1H), 8.01 (d, J=12.5 Hz, 1H), 7.47-7.42 (m, 1H), 7.30 (d, J=6.9 Hz, 1H), 7.20 (d, J=9.8 Hz, 1H), 4.85 (p, J=7.0 Hz, 1H), 4.09 (s, 3H), 3.52 (s, 2H), 3.43-3.35 (m, 2H), 2.82-2.74 (m, 2H), 2.66 (dq, J=11.9, 6.3 Hz, 2H), 2.10 (dq, J=12.6, 6.4 Hz, 1H), 1.93 (s, 2H), 1.84 (dt, J=12.2, 6.4 Hz, 1H).

### Example 74: Synthesis of Compounds 277 and 278

### Synthesis of Intermediate B188

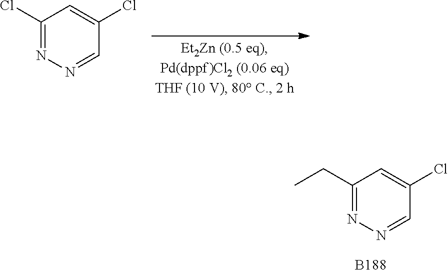

A solution of 3,5-dichloropyridazine (5 g, 33.564 mmol, 1 equiv) and ZnEt₂ (2.07 g, 16.782 mmol, 0.5 equiv), Pd(dppf)Cl₂ (1.47 g, 2.014 mmol, 0.06 equiv) in THE (50 mL) was stirred for 2 h at 80° C. under nitrogen atmosphere. The mixture was allowed to cool down to room temperature. The resulting mixture was poured into NH₄C1(aq). The resulting mixture was extracted with EA (1×150 mL). dried over anhydrous Na₂SO₄. After filtration, the filtrate was concentrated under reduced pressure. The residue was purified by silica gel column chromatography, eluted with PE/EA (5:1) to afford 5-chloro-3-ethylpyridazine (1 g, 21%) as a solid. LCMS:(ES, m/z):143[M+H]⁺

### Synthesis of Intermediate B189

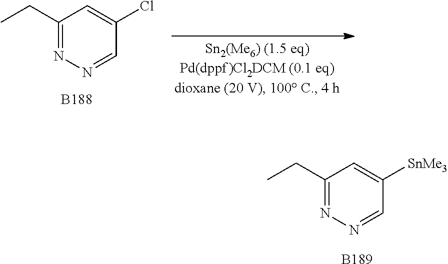

A solution of 5-chloro-3-ethylpyridazine (900 mg, 6.312 mmol, 1 equiv) and Pd(dppf)Cl₂·CH₂Cl₂ (514.17 mg, 0.631 mmol, 0.1 equiv) in 1,4-dioxane (18 mL) was stirred for 4 h at 100° C. under nitrogen atmosphere. The mixture was allowed to cool down to room temperature. The resulting mixture was poured into KF(aq). The resulting mixture was extracted with EA (1×50 mL). dried over anhydrous Na₂SO₄. After filtration, the filtrate was concentrated under reduced pressure. This resulted in 3-ethyl-5-(trimethylstannyl)pyridazine (2 g, 116.95%) as a solid. LCMS:(ES, m/z):273[M+H]⁺

### Synthesis of Intermediate B190

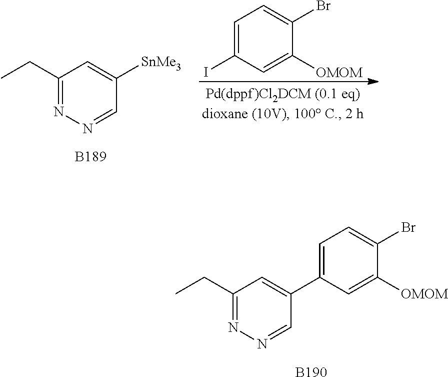

A solution of 1-bromo-4-iodo-2-(methoxymethoxy)benzene (1.25 g, 3.645 mmol, 1 equiv) in 1,4-dioxane (12.5 mL) was treated with Pd(dppf)Cl₂·CH₂Cl₂ (0.30 g, 0.365 mmol, 0.1 equiv) at 100° C. under nitrogen atmosphere followed by the addition of 3-ethyl-5-(trimethylstannyl)pyridazine (1.98 g, 7.290 mmol, 2 equiv) dropwise at 100° C., was stirred for 2 h at 100° C. under nitrogen atmosphere. The mixture was allowed to cool down to room temperature. The resulting mixture was poured into water. The resulting mixture was extracted with EA (1×40 mL). dried over anhydrous Na₂SO₄. After filtration, the filtrate was concentrated under reduced pressure. The residue was purified by silica gel column chromatography, eluted with PE/EA (1:1) to afford 5-[4-bromo-3-(methoxymethoxy)phenyl]-3-ethylpyridazine (600 mg, 51%) as a solid. LCMS:(ES, m/z):323[M+H]⁺

### Synthesis of Intermediate B191

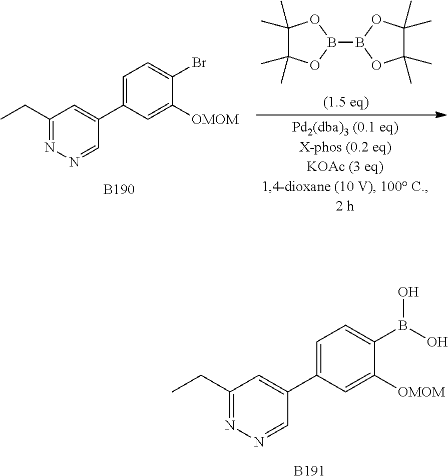

A solution of 5-[4-bromo-3-(methoxymethoxy)phenyl]-3-ethylpyridazine (540 mg, 1.671 mmol, 1 equiv) and bis(pinacolato)diboron (636 mg, 2.506 mmol, 1.5 equiv), Pd₂(dba)₃ (153.0 mg, 0.167 mmol, 0.1 equiv), XPhos (159 mg, 0.334 mmol, 0.2 equiv) KOAc (492 mg, 5.013 mmol, 3 equiv) in 1,4-dioxane (5.4 mL) was stirred for 2 h at 100° C. under nitrogen atmosphere.

The mixture was allowed to cool down to room temperature. The resulting mixture was poured into water. The resulting mixture was extracted with EA (1×20 mL). dried over anhydrous Na₂SO₄. After filtration, the filtrate was concentrated under reduced pressure. This resulted in 4-(6-ethylpyridazin-4-yl)-2-(methoxymethoxy)phenylboronic acid (600 mg, 124%) as an oil.

LCMS:(ES, m/z):289[M+H]⁺

### Synthesis of Intermediate B192

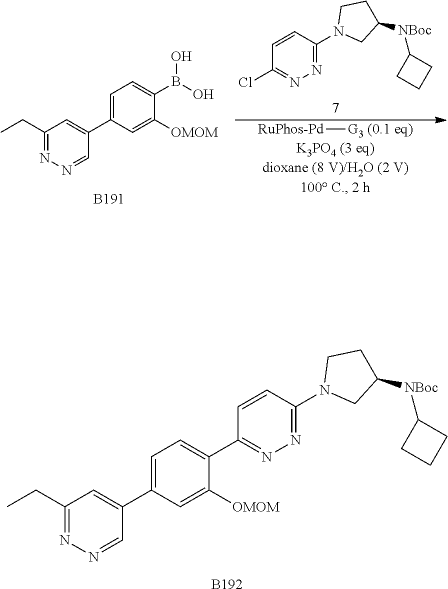

A solution of tert-butyl N-[(3R)-1-(6-chloropyridazin-3-yl)pyrrolidin-3-yl]-N-cyclobutylcarbamate (100 mg, 0.283 mmol, 1 equiv) and 4-(6-ethylpyridazin-4-yl)-2-(methoxymethoxy)phenylboronic acid (163 mg, 0.566 mmol, 2 equiv), K₃PO₄ (181 mg, 0.849 mmol, 3 equiv),RuPhos Palladacycle Gen.3 (24 mg, 0.028 mmol, 0.1 equiv) in 1,4-dioxane (0.8 mL), water (0.2 mL) was stirred for 2 h at 100° C. under nitrogen atmosphere. The mixture was allowed to cool down to room temperature. The resulting mixture was poured into water. The resulting mixture was extracted with EA (1×5 mL). dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-cyclobutyl-N-[(3R)-1-{6-[4-(6-ethylpyridazin-4-yl)-2-(methoxymethoxy)phenyl]pyridazin-3-yl}pyrrolidin-3-yl]carbamate (95 mg, 60%) as a solid. LCMS:(ES, m/z):561[M+H]⁺

### Synthesis of Compound 277

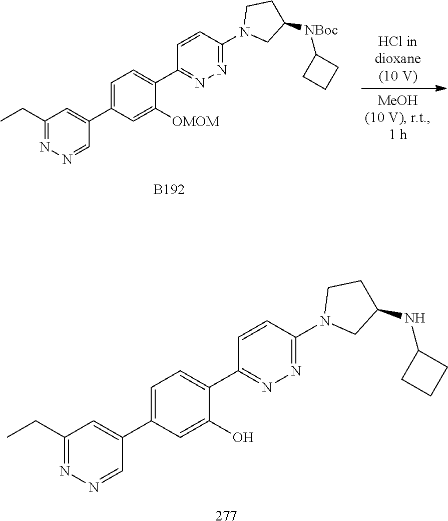

A solution of tert-butyl N-cyclobutyl-N-[(3R)-1-{6-[4-(6-ethylpyridazin-4-yl)-2-(methoxymethoxy)phenyl]pyridazin-3-yl}pyrrolidin-3-yl]carbamate (95 mg, 0.169 mmol, 1 equiv) in MeOH (0.95 mL), HCl(gas) in 1,4-dioxane (0.95 mL) was stirred for 1 h at room temperature. The resulting mixture was concentrated under reduced pressure. The crude product was purified by Prep-HPLC (Condition 8, Gradient 1) to afford 2-{6-[(3R)-3-(cyclobutylamino)pyrrolidin-1-yl]pyridazin-3-yl}-5-(6-ethylpyridazin-4-yl)phenol (15.9 mg, 22%) as a solid. LCMS:(ES, m/z):417[M+H]^{+ 1}HNMR: (400 MHz, DMSO-d₆) δ 14.11 (s, 1H), 9.51 (d, J=2.3 Hz, 1H), 8.31 (d, J=9.8 Hz, 1H), 8.06 (d, J=8.4 Hz, 1H), 7.95 (d, J=2.3 Hz, 1H), 7.55-7.46 (m, 2H), 7.18 (d, J=9.8 Hz, 1H), 3.64 (dt, J=15.4, 7.6 Hz, 2H), 3.54-3.45 (m, 1H), 3.43-3.35 (m, 1H), 3.32-3.21 (m, 2H), 3.00 (q, J=7.6 Hz, 2H), 2.13 (s, 4H), 1.83 (dq, J=13.1, 6.6 Hz, 1H), 1.68 (s, 2H), 1.66-1.50 (m, 2H), 1.35 (t, J=7.6 Hz, 3H).

### Example 75: Synthesis of Compounds 279 and 280

### Synthesis of Intermediate B193

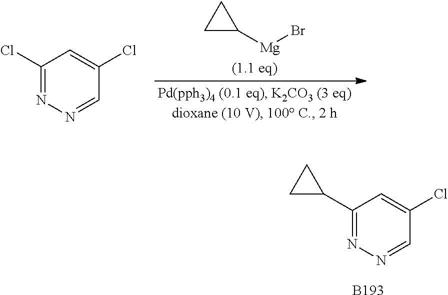

A solution of 3,5-dichloropyridazine (5 g, 33.564 mmol, 1 equiv) and bromo(cyclopropyl)magnesium (5.85 g, 40.277 mmol, 1.2 equiv), Pd(PPh₃)₄(3.88 g, 3.356 mmol, 0.1 equiv)), K₂CO₃ (13.92 g, 100.692 mmol, 3 equiv) in 1,4-dioxane (50 mL) was stirred for 2 h at 100° C. under nitrogen atmosphere. The mixture was allowed to cool down to room temperature. The resulting mixture was poured into NH₄Cl(aq). The resulting mixture was extracted with EA (1×150 mL). dried over anhydrous Na₂SO₄. After filtration, the filtrate was concentrated under reduced pressure. The residue was purified by silica gel column chromatography, eluted with PE/EA (5:1) to afford 5-chloro-3-cyclopropylpyridazine (346 mg, 7%) as an oil. LCMS:(ES, m/z):155

### Synthesis of Intermediate B194

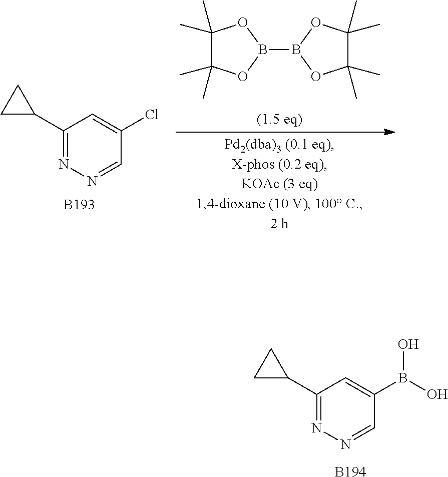

A solution of 5-chloro-3-cyclopropylpyridazine (306 mg, 1.979 mmol, 1 equiv) and bis(pinacolato)diboron (754 mg, 2.969 mmol, 1.5 equiv), Pd₂(dba)₃ (181 mg, 0.198 mmol, 0.1 equiv), XPhos (189 mg, 0.396 mmol, 0.2 equiv), KOAc (583 mg, 5.937 mmol, 3 equiv) in 1,4-dioxane (3 mL) was stirred for 2 h at 100° C. under nitrogen atmosphere. The mixture was allowed to cool down to room temperature. The resulting mixture was poured into water. The resulting mixture was extracted with EA (1×10 mL). dried over anhydrous Na₂SO₄. After filtration, the filtrate was concentrated under reduced pressure. This resulted in 6-cyclopropylpyridazin-4-ylboronic acid (350 mg, 108%) as a oil. LCMS:(ES, m/z):165

### Synthesis of Intermediate B195

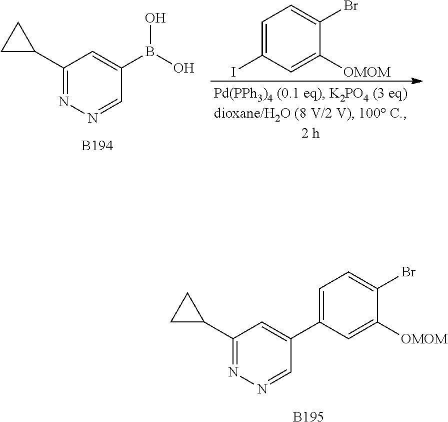

A solution of 1-bromo-4-iodo-2-(methoxymethoxy)benzene (348 mg, 1.015 mmol, 1 equiv) and 6-cyclopropylpyridazin-4-ylboronic acid (333 mg, 2.030 mmol, 2 equiv), Pd(PPh₃)₄(117 mg, 0.101 mmol, 0.1 equiv), K₃PO₄ (646 mg, 3.045 mmol, 3 equiv) in 1,4-dioxane (2.8 mL), water (0.7 mL) was stirred for 2 h at 100° C. under nitrogen atmosphere. The mixture was allowed to cool down to room temperature. The resulting mixture was poured into water. The resulting mixture was extracted with EA (1×10 mL) and dried over anhydrous Na₂SO₄. After filtration, the filtrate was concentrated under reduced pressure. The residue was purified by silica gel column chromatography, eluted with PE/EA (5:1) to afford 5-[4-bromo-3-(methoxymethoxy)phenyl]-3-cyclopropylpyridazine (244 mg, 72%) as a solid. LCMS:(ES, m/z):335

### Synthesis of Intermediate B196

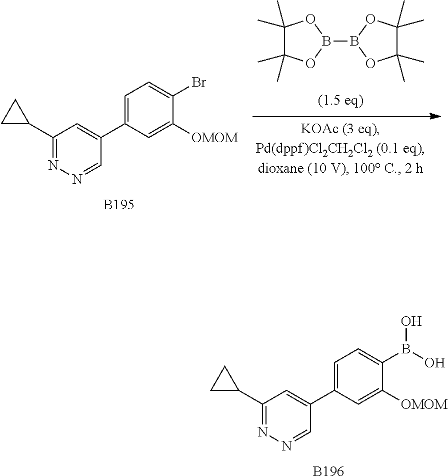

A solution of 5-[4-bromo-3-(methoxymethoxy)phenyl]-3-cyclopropylpyridazine (220 mg, 0.656 mmol, 1 equiv) and bis(pinacolato)diboron (250.0 mg, 0.984 mmol, 1.5 equiv), Pd(dppf)Cl₂·CH₂Cl₂ (54 mg, 0.066 mmol, 0.1 equiv), KOAc (193 mg, 1.968 mmol, 3 equiv) in 1,4-dioxane (2.2 mL) was stirred for 2 h at 100° C. under nitrogen atmosphere. The mixture was allowed to cool down to room temperature. The resulting mixture was poured into water. The resulting mixture was extracted with EA (1×10 mL). dried over anhydrous Na₂SO₄. After filtration, the filtrate was concentrated under reduced pressure. This resulted in 4-(6-cyclopropylpyridazin-4-yl)-2-(methoxymethoxy)phenylboronic acid (250 mg, 127%) as an oil. LCMS:(ES, m/z):301

### Synthesis of Intermediate 197

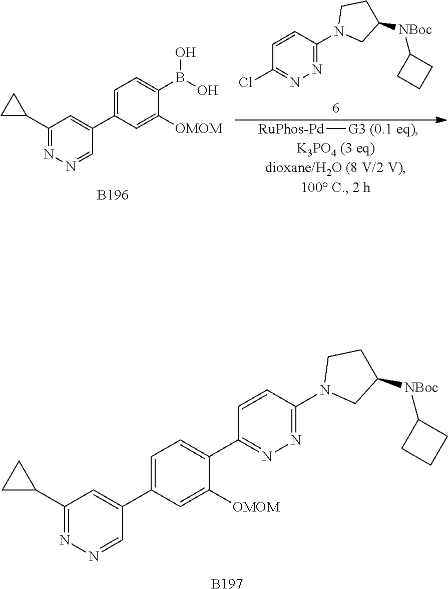

A solution of tert-butyl N-[(3R)-1-(6-chloropyridazin-3-yl)pyrrolidin-3-yl]-N-cyclobutylcarbamate (60 mg, 0.170 mmol, 1 equiv) and 4-(6-cyclopropylpyridazin-4-yl)-2-(methoxymethoxy)phenylboronic acid (102 mg, 0.340 mmol, 2 equiv), RuPhos Palladacycle Gen.3 (14 mg, 0.017 mmol, 0.1 equiv), K₃PO₄ (108 mg, 0.510 mmol, 3 equiv) in 1,4-dioxane (0.48 mL), water (0.12 mL) was stirred for 2 h at 100° C. under nitrogen atmosphere. The mixture was allowed to cool down to room temperature. The resulting mixture was extracted with EA (1×2 mL) and dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-cyclobutyl-N-[(3R)-1-{6-[4-(6-cyclopropylpyridazin-4-yl)-2-(methoxymethoxy)phenyl]pyridazin-3-yl}pyrrolidin-3-yl]carbamate (35 mg, 36%) as a solid. LCMS:(ES, m/z):573

### Synthesis of Compound 279

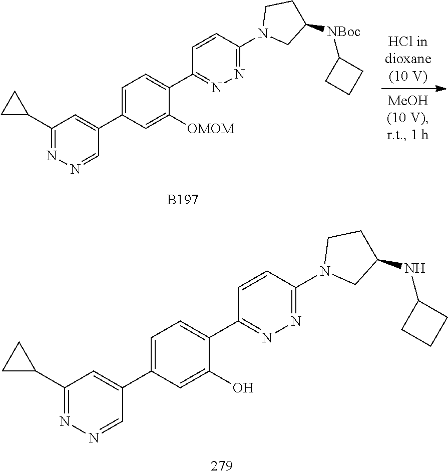

A solution of tert-butyl N-cyclobutyl-N-[(3R)-1-{6-[4-(6-cyclopropylpyridazin-4-yl)-2-(methoxymethoxy)phenyl]pyridazin-3-yl}pyrrolidin-3-yl]carbamate (35 mg, 0.061 mmol, 1 equiv) in MeOH (0.35 mL) HCl(gas) in 1,4-dioxane (0.35 mL) was stirred for 1 h at room temperature. The resulting mixture was concentrated under reduced pressure. The crude product was purified by Prep-HPLC (Condition 6, Gradient 1) to afford 2-{6-[(3R)-3-(cyclobutylamino)pyrrolidin-1-yl]pyridazin-3-yl}-5-(6-cyclopropylpyridazin-4-yl)phenol (5.3 mg, 20%) as a solid. LCMS:(ES, m/z):429 ¹H NMR: (400 MHz, DMSO-d₆) δ 14.11 (s, 1H), 9.43 (d, J=2.3 Hz, 1H), 8.30 (d, J=9.9 Hz, 1H), 8.05 (d, J=8.3 Hz, 1H), 7.84 (d, J=2.3 Hz, 1H), 7.54-7.45 (m, 2H), 7.18 (d, J=9.8 Hz, 1H), 3.64 (dt, J=14.9, 7.5 Hz, 2H), 3.51 (d, J=8.3 Hz, 1H), 3.39 (t, J=5.7 Hz, 1H), 3.31-3.21 (m, 2H), 2.18-2.14 (S, 1H), 2.13 (s, 4H), 1.83 (dq, J=13.0, 6.7 Hz, 1H), 1.74-1.63 (m, 2H), 1.63-1.50 (m, 2H), 1.21-1.08 (m, 4H).

### Synthesis of Intermediate B]79

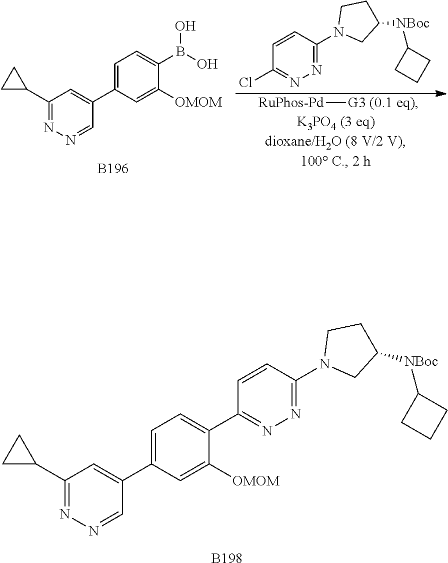

A solution of tert-butyl N-[(3S)-1-(6-chloropyridazin-3-yl)pyrrolidin-3-yl]-N-cyclobutylcarbamate (60 mg, 0.170 mmol, 1 equiv) and 4-(6-cyclopropylpyridazin-4-yl)-2-(methoxymethoxy)phenylboronic acid (102 mg, 0.340 mmol, 2 equiv), RuPhos Palladacycle Gen.3 (14 mg, 0.017 mmol, 0.1 equiv),K₃PO₄ (108 mg, 0.510 mmol, 3 equiv) in 1,4-dioxane (0.48 mL), water (0.12 mL) was stirred for 2 h at 100° C. under nitrogen atmosphere. The mixture was allowed to cool down to room temperature. The resulting mixture was extracted with EtOAc (1×2 mL). dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-cyclobutyl-N-[(3S)-1-{6-[4-(6-cyclopropylpyridazin-4-yl)-2-(methoxymethoxy)phenyl]pyridazin-3-yl}pyrrolidin-3-yl]carbamate (45 mg, 46%) as a solid. LCMS:(ES, m/z):573

### Synthesis of Compound 280

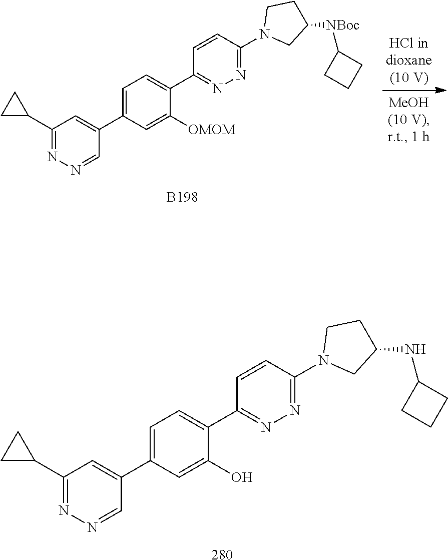

A solution of tert-butyl N-cyclobutyl-N-[(3S)-1-{6-[4-(6-cyclopropylpyridazin-4-yl)-2-(methoxymethoxy)phenyl]pyridazin-3-yl}pyrrolidin-3-yl]carbamate (45 mg, 0.079 mmol, 1 equiv) in MeOH (0.45 mL), HCl(gas) in 1,4-dioxane (0.45 mL) was stirred for 1 h at room temperature. The resulting mixture was concentrated under reduced pressure. The crude product was purified by Prep-HPLC (Condition 6, Gradient 1) to afford 2-{6-[(3S)-3-(cyclobutylamino)pyrrolidin-1-yl]pyridazin-3-yl}-5-(6-cyclopropylpyridazin-4-yl)phenol (12.1 mg, 36%) as a solid. LCMS:(ES, m/z):429 ¹H NMR: (400 MHz, DMSO-d₆) δ 14.11 (s, 1H), 9.43 (d, J=2.2 Hz, 1H), 8.30 (d, J=9.9 Hz, 1H), 8.05 (d, J=8.4 Hz, 1H), 7.84 (d, J=2.3 Hz, 1H), 7.57-7.44 (m, 2H), 7.17 (d, J=9.8 Hz, 1H), 3.69-3.58 (m, 2H), 3.50 (q, J=8.6, 7.9 Hz, 1H), 3.39 (q, J=5.6 Hz, 1H), 3.26 (dd, J=15.2, 7.6 Hz, 2H), 2.38-2.27 (m, 1H), 2.19-2.03 (m, 4H), 1.83 (dq, J=13.0, 6.7 Hz, 1H), 1.69 (tdd, J=11.2, 7.3, 4.2 Hz, 2H), 1.66-1.49 (m, 2H), 1.21-1.08 (m, 4H).

### Example 76: Synthesis of Compounds 282 and 283

### Synthesis of Intermediate B199

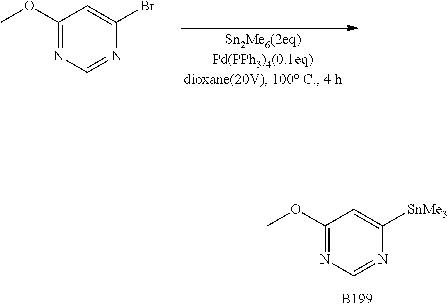

A solution of 4-bromo-6-methoxypyrimidine (500 mg, 2.645 mmol, 1 equiv) and Pd(PPh₃)₄ (306 mg, 0.265 mmol, 0.1 equiv), Sn₂Me₆ (1733 mg, 5.290 mmol, 2 equiv) in 1,4-dioxane (10 mL) was stirred for 4 h at 100° C. under nitrogen atmosphere. The mixture was allowed to cool down to room temperature. The resulting mixture was poured into KF(aq). The resulting mixture was extracted with EA (1×50 mL). The combined organic layers were washed with brine (1×20 mL), dried over anhydrous Na₂SO₄. After filtration, the filtrate was concentrated under reduced pressure. This resulted in 4-methoxy-6-(trimethylstannyl)pyrimidine (1.1 g, 152%) as a solid. LCMS:(ES, m/z):275

### Synthesis of Intermediate B200

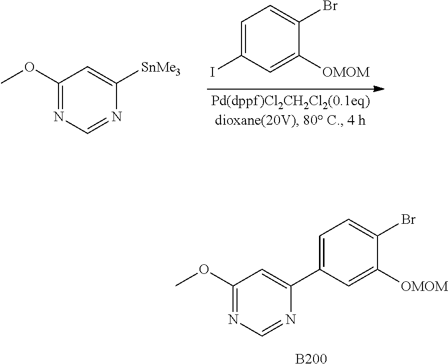

To a stirred solution of 1-bromo-4-iodo-2-(methoxymethoxy)benzene (600 mg, 1.749 mmol, 1 equiv) and Pd(dppf)Cl₂CH₂Cl₂ (143 mg, 0.175 mmol, 0.1 equiv) in 1,4-dioxane (12 mL) was added 4-methoxy-6-(trimethylstannyl)pyrimidine (955 mg, 3.498 mmol, 2 equiv) dropwise at 80° C. under nitrogen atmosphere. The reaction of 4 h. The mixture was allowed to cool down to room temperature. The resulting mixture was poured into water. The resulting mixture was extracted with EA (1×30 mL). dried over anhydrous Na₂SO₄. After filtration, the filtrate was concentrated under reduced pressure. The residue was purified by silica gel column chromatography, eluted with PE/EA (1:1) to afford 4-[4-bromo-3-(methoxymethoxy)phenyl]-6-methoxypyrimidine (264 mg, 46%) as a solid. LCMS:(ES, m/z):325

### Synthesis of Intermediate B201

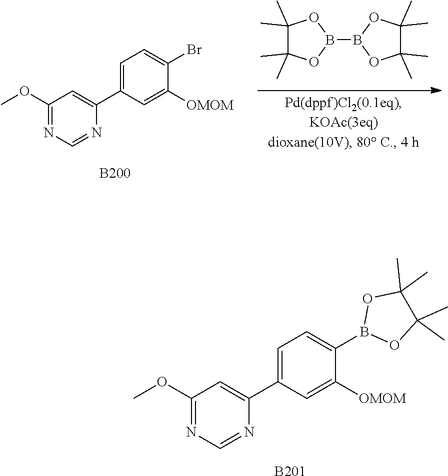

A solution of 4-[4-bromo-3-(methoxymethoxy)phenyl]-6-methoxypyrimidine (230 mg, 0.707 mmol, 1 equiv) and bis(pinacolato)diboron (269 mg, 1.060 mmol, 1.5 equiv), KOAc (208 mg, 2.121 mmol, 3 equiv) Pd(dppf)Cl₂CH₂Cl₂ (58 mg, 0.071 mmol, 0.1 equiv) in 1,4-dioxane (2.3 mL) was stirred for 4 h at 80° C. under nitrogen atmosphere. The mixture was allowed to cool down to room temperature. The resulting mixture was poured into water. The resulting mixture was extracted with EA (1×6 mL). dried over anhydrous Na₂SO₄. After filtration, the filtrate was concentrated under reduced pressure. This resulted in 4-methoxy-6-[3-(methoxymethoxy)-4-(4,4,5,5-tetramethyl-1,3,2-dioxaborolan-2-yl)phenyl]pyrimidine (200 mg, 76%) as an oil. LCMS:(ES, m/z):373

### Synthesis of Intermediate B203

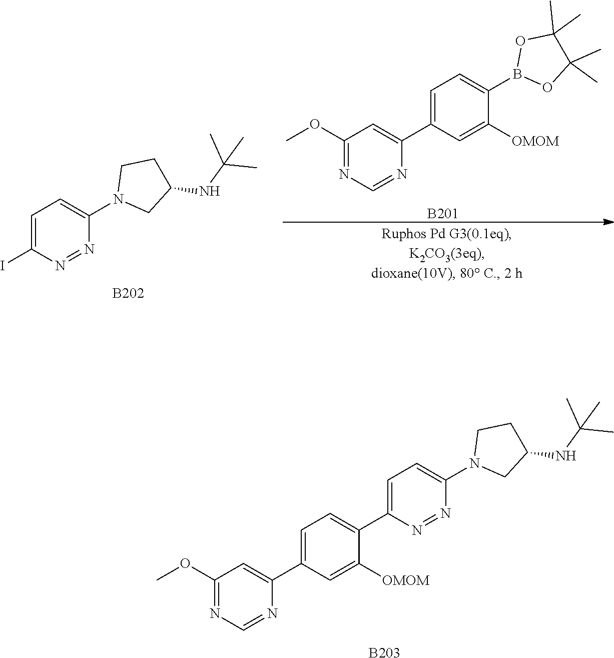

A solution of (3R)—N-tert-butyl-1-(6-iodopyridazin-3-yl)pyrrolidin-3-amine (60 mg, 0.173 mmol, 1 equiv) and 4-methoxy-6-[3-(methoxymethoxy)-4-(4,4,5,5-tetramethyl-1,3,2-dioxaborolan-2-yl)phenyl]pyrimidine (129 mg, 0.346 mmol, 2 equiv), K₂CO₃ (72 mg, 0.519 mmol, 3 equiv), RuPhos Palladacycle Gen.3 (15 mg, 0.017 mmol, 0.1 equiv) in 1,4-dioxane (0.5 mL), water (0.1 mL) was stirred for 2 h at 80° C. under nitrogen atmosphere. The mixture was allowed to cool down to room temperature. The resulting mixture was extracted with EA (1×3 mL). dried over anhydrous Na₂SO₄. After filtration, the filtrate was concentrated under reduced pressure. The residue was purified by silica gel column chromatography, eluted with CH₂Cl₂/MeOH (10:1) to afford (3R)—N-tert-butyl-1-{6-[2-(methoxymethoxy)-4-(6-methoxypyrimidin-4-yl)phenyl]pyridazin-3-yl}pyrrolidin-3-amine (20 mg, 25%) as a solid. LCMS:(ES, m/z):465

### Synthesis of Intermediate B205

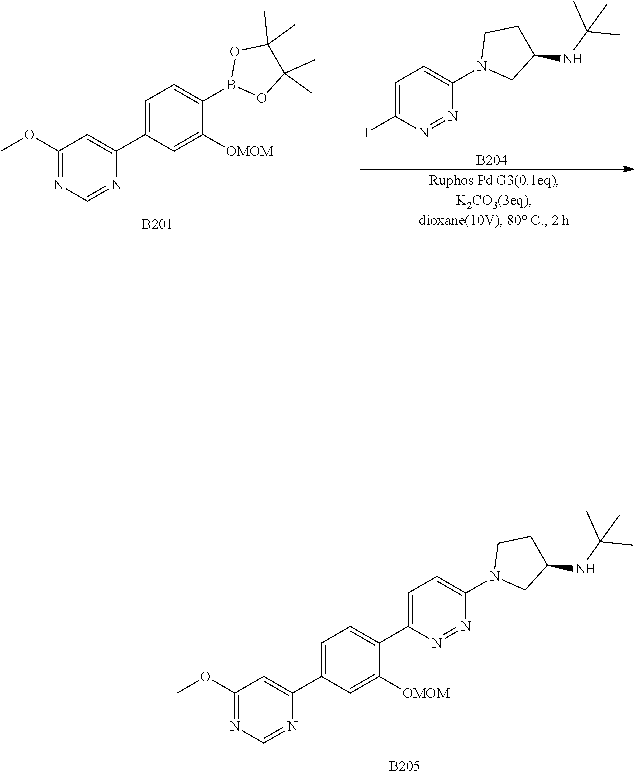

A solution of 4-methoxy-6-[3-(methoxymethoxy)-4-(4,4,5,5-tetramethyl-1,3,2-dioxaborolan-2-yl)phenyl]pyrimidine (112.89 mg, 0.303 mmol, 1.5 equiv) in dioxane (5 mL)/H₂O (1 mL) was treated with (3R)—N-tert-butyl-1-(6-iodopyridazin-3-yl)pyrrolidin-3-amine (70 mg, 0.202 mmol, 1.00 equiv), K₂CO₃ (83.83 mg, 0.606 mmol, 3 equiv) and RuPhos Palladacycle Gen.3 (16.91 mg, 0.020 mmol, 0.1 equiv) for 2 h at 80° C. under nitrogen atmosphere. The resulting mixture was filtered and the filter cake was washed with EtOAc (3×3 mL). The aqueous layer was extracted with EtOAc (3×2 mL). The combined organic layers were dried over anhydrous Na₂SO₄. After filtration, the filtrate was concentrated under reduced pressure. The residue was purified by Prep-TLC (CH₂Cl₂/MeOH 10:1) to afford (3R)—N-tert-butyl-1-{6-[2-(methoxymethoxy)-4-(6-methoxypyrimidin-4-yl)phenyl]pyridazin-3-yl}pyrrolidin-3-amine (20 mg, 21%) as an oil. LCMS:(ES, m/z): 465 [M+H]⁺

### Synthesis of Compound 282

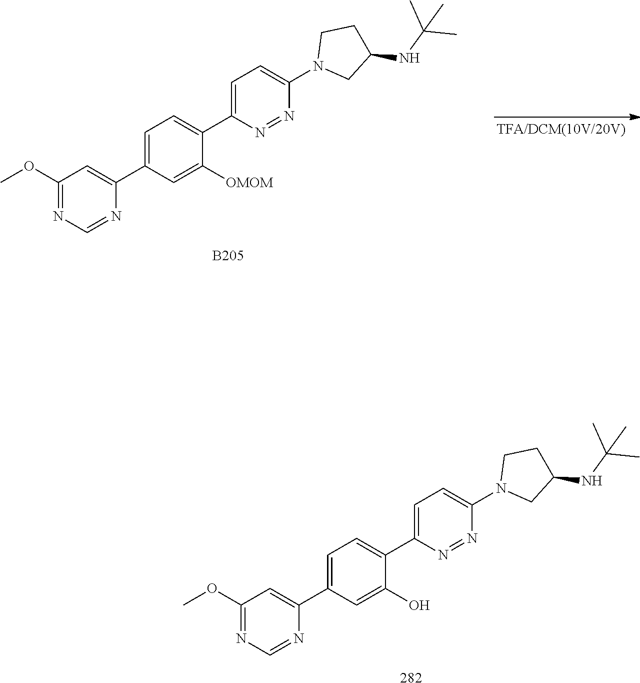

Into a 10 mL vial were added (3R)—N-tert-butyl-1-{6-[2-(methoxymethoxy)-4-(6-methoxypyrimidin-4-yl)phenyl]pyridazin-3-yl}pyrrolidin-3-amine (20 mg, 0.043 mmol, 1 equiv). TFA (0.5 mL) and DCM (1.5 mL) at room temperature. A mixture was stirred for 2 h at room temperature. The resulting mixture was concentrated under reduced pressure. The crude product was purified by Chiral-Prep-HPLC (Condition 10, Gradient 3) to afford 2-{6-[(3R)-3-(tert-butylamino)pyrrolidin-1-yl]pyridazin-3-yl}-5-(6-methoxypyrimidin-4-yl)phenol (2 mg, 10.77%) as a solid. LCMS:(ES, m/z):421[M+H]^{+ 1}H NMR: (400 MHz, DMSO-d₆) δ 13.92 (s, 1H), 8.87 (s, 1H), 8.27 (d, J=10.0 Hz, 1H), 8.04-7.97 (m, 1H), 7.74 (d, J=7.3 Hz, 2H), 7.52 (d, J=3.0 Hz, 1H), 7.17 (d, J=9.8 Hz, 1H), 3.99 (s, 3H), 3.80 (s, 1H), 3.65 (s, 1H), 3.54 (s, 1H), 6 3.44 (d, J=9.4 Hz, 1H), 3.09 (s, 1H), 2.18 (q, J=8.3, 6.6 Hz, 1H), 1.77 (dq, J=12.4, 8.5 Hz, 1H), 1.09 (s, 9H).

### Synthesis of Compound 283

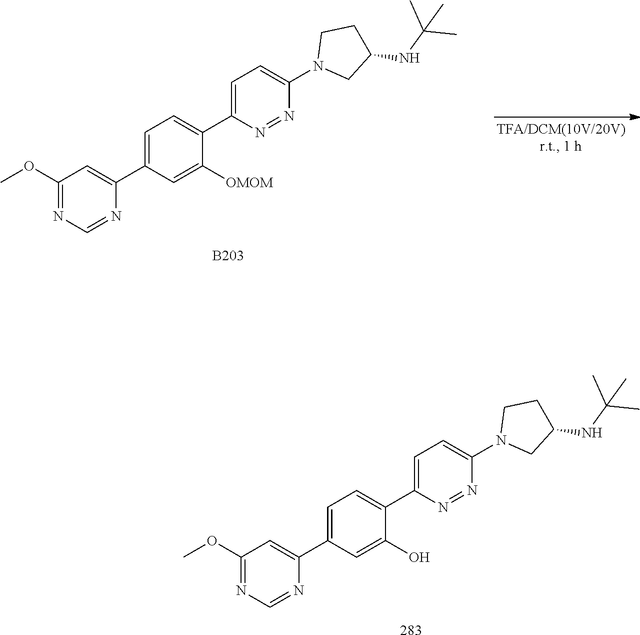

A solution of (3S)—N-tert-butyl-1-{6-[2-(methoxymethoxy)-4-(6-methoxypyrimidin-4-yl)phenyl]pyridazin-3-yl}pyrrolidin-3-amine (35 mg, 0.075 mmol, 1 equiv) in DCM (0.7 mL). TFA (0.35 mL) was stirred for 1 h at room temperature. The resulting mixture was concentrated under reduced pressure. The crude product was purified by Prep-HPLC (Condition 8, Gradient 1) to afford 2-{6-[(3S)-3-(tert-butylamino)pyrrolidin-1-yl]pyridazin-3-yl}-5-(6-methoxypyrimidin-4-yl)phenol (10.5 mg, 33%) as a solid. LCMS:(ES, m/z):421[M+H]^{+ 1}HNMR: (400 MHz, DMSO-d₆) δ 13.92 (s, 1H), 8.87 (d, J=1.0 Hz, 1H), 8.28 (d, J=9.8 Hz, 1H), 8.05-7.99 (m, 1H), 7.77-7.71 (m, 2H), 7.53 (d, J=1.1 Hz, 1H), 7.17 (d, J=9.7 Hz, 1H), 3.99 (s, 3H), 3.80 (s, 1H), 3.66 (s, 1H), 3.55 (s, 1H), 3.45 (s, 1H), 3.10 (s, 1H), 2.19 (s, 1H), 1.77 (s, 1H), 1.09 (s, 9H).

### Example 77: Synthesis of Compound 285

### Synthesis of Intermediate B204

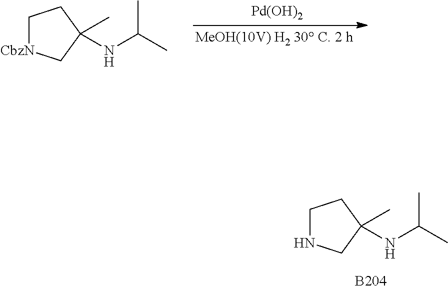

A mixture of benzyl 3-(isopropylamino)-3-methylpyrrolidine-1-carboxylate (632 mg, 2.287 mmol, 1 equiv) and Pd(OH)₂ (64 mg, 0.457 mmol, 0.2 equiv) in MeOH (6 mL) was stirred for 2 h at 30° C. under hydrogen atmosphere. The resulting mixture was filtered and the filter cake was washed with DCM (3×5 mL). The filtrate was concentrated under reduced pressure. This resulted in N-isopropyl-3-methylpyrrolidin-3-amine (217 mg, 67%) as a solid. LCMS:(ES, m/z):143 [M+H]+

### Synthesis of Intermediate B205

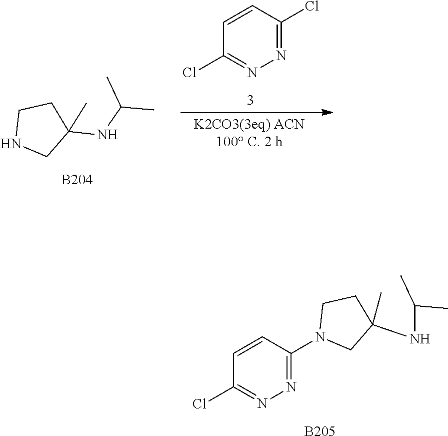

A solution of N-isopropyl-3-methylpyrrolidin-3-amine (217 mg, 1.526 mmol, 1.00 equiv) and pyridazine, 3,6-dichloro- (227 mg, 1.526 mmol, 1 equiv) K₂CO₃ (638 mg 4.538 mmol, 3.00 equiv) in ACN (2 mL, 15.260 mmol, 10 equiv) was stirred for 2 h at 100° C. The mixture was allowed to cool down to room temperature. The resulting mixture was filtered and the filter cake was washed with MeOH (4×5 mL). The filtrate was concentrated under reduced pressure. The residue was purified by silica gel column chromatography, eluted with PE/EA (1:1) to afford 1-(6-chloropyridazin-3-yl)-N-isopropyl-3-methylpyrrolidin-3-amine (210 mg, 54%) as a solid.

LCMS:(ES, m/z):255 [M+H]+

### Synthesis of Intermediate B206

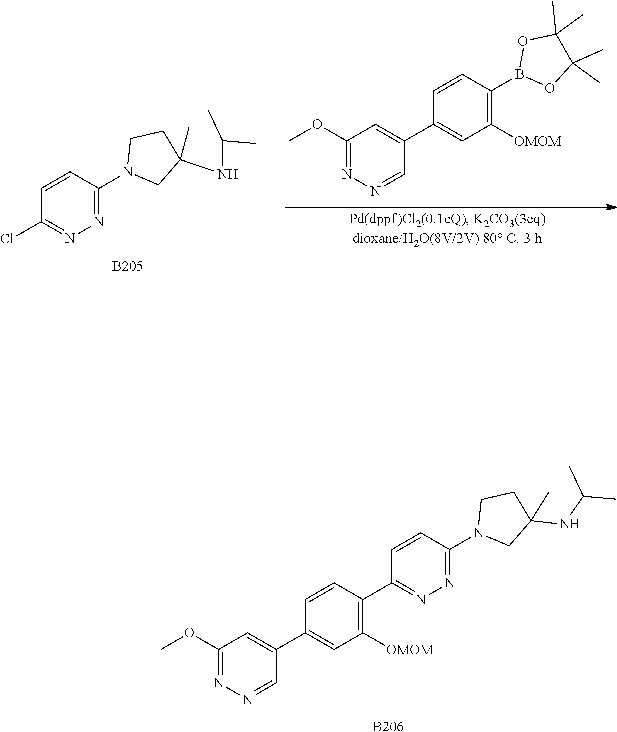

A mixture of 1-(6-chloropyridazin-3-yl)-N-isopropyl-3-methylpyrrolidin-3-amine (100 mg, 0.393 mmol, 1 equiv) and 3-methoxy-5-[3-(methoxymethoxy)-4-(4,4,5-trimethyl-1,3,2-dioxaborolan-2-yl)phenyl]pyridazine (182 mg, 0.511 mmol, 1.3 equiv), Pd(dppf)Cl₂ (40 mg, 49.746 mmol, 0.1 equiv) K₂CO₃ (162 mg, 1.179 mmol, 3 equiv) in 1,4-dioxane (5 mL), H₂O (1.25 mL) was stirred for 3 h at 80° C. under nitrogen atmosphere. The mixture was allowed to cool down to room temperature. The resulting mixture was filtered and the filter cake was washed with DCM (5×5 mL). The filtrate was concentrated under reduced pressure. The residue was purified by silica gel column chromatography, eluted with PE/EA (1:1) to afford N-isopropyl-1-{6-[2-(methoxymethoxy)-4-(6-methoxypyridazin-4-yl)phenyl]pyridazin-3-yl}-3-methylpyrrolidin-3-amine (120 mg, 66%) as an oil. LCMS:(ES, m/z):465 [M+H]+

### Synthesis of Compound 285

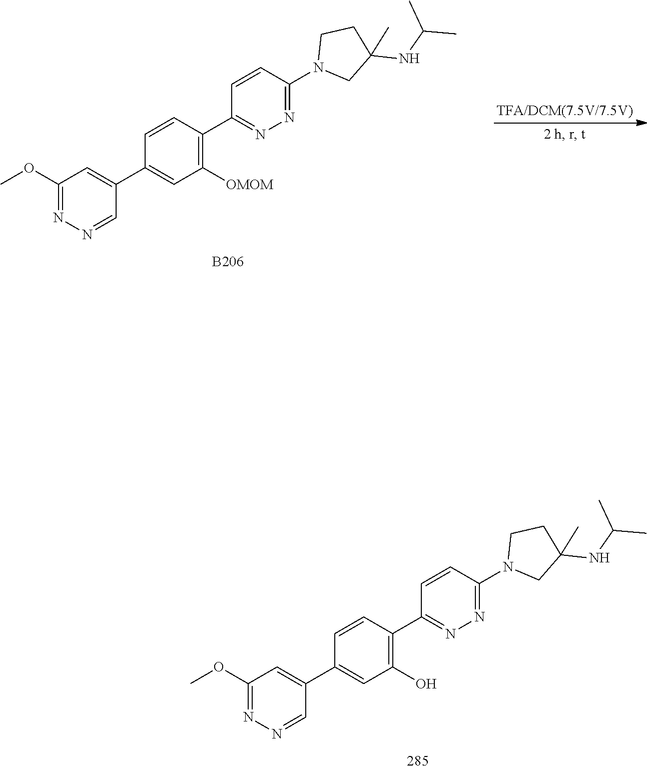

A mixture of N-isopropyl-1-{6-[2-(methoxymethoxy)-4-(6-methoxypyridazin-4-yl)phenyl]pyridazin-3-yl}-3-methylpyrrolidin-3-amine (100 mg, 0.215 mmol, 1.00 equiv) and trifluoroacetaldehyde (0.75 mL, 0.003 mmol, 1 equiv) in DCM was stirred for 2 h at room temperature. The resulting mixture was concentrated under reduced pressure. The crude product was purified by Prep-HPLC (Condition 6, Gradient 1) to afford 2-{6-[3-(isopropylamino)-3-methylpyrrolidin-1-yl]pyridazin-3-yl}-5-(6-methoxypyridazin-4-yl)phenol (38.2 mg, 42%) as a solid. LCMS:(ES, m/z):421 [M+H] +NMR: (400 MHz, DMSO-d₆) δ 14.12 (s, 1H), 9.35 (d, J=1.9 Hz, 1H), 8.30 (d, J=9.8 Hz, 1H), 8.04 (d, J=8.3 Hz, 1H), 7.56 (d, J=1.9 Hz, 1H), 7.52-7.44 (m, 2H), 7.17 (d, J=9.7 Hz, 1H), 4.08 (s, 3H), 3.64 (s, 1H), 3.54 (d, J=9.3 Hz, 1H), 3.4(s, 2H), 2.96-2.78 (m, 1H), 2.00 (dt, J=12.2, 7.3 Hz, 1H), 1.86 (ddd, J=12.6, 7.6, 5.7 Hz, 1H), 1.25 (s, 3H), 1.02 (dd, J=11.7, 6.3 Hz, 6H).

### Example 78: Synthesis of Compounds 158 and 159

### Synthesis of Intermediate B207

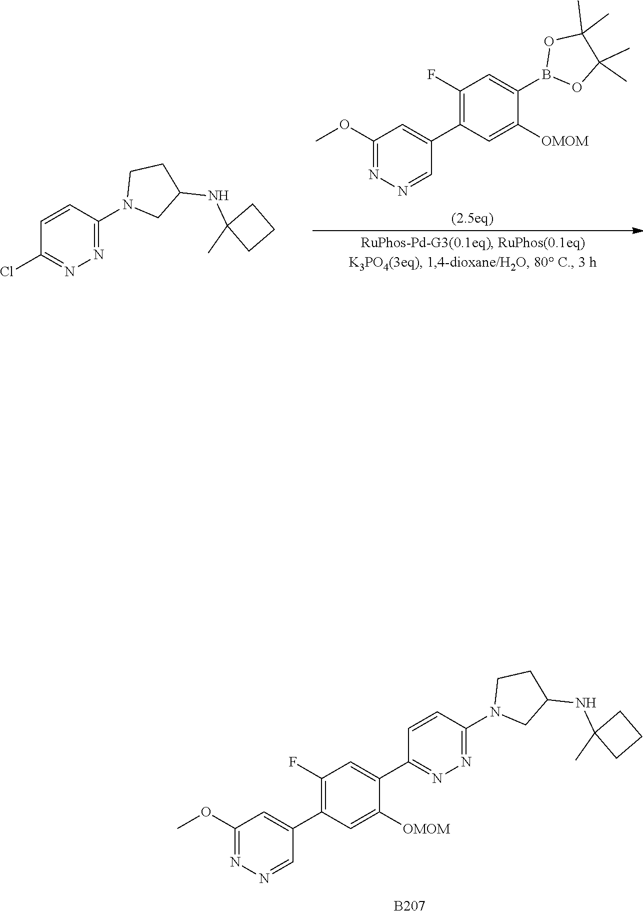

A solution of 5-[2-fluoro-5-(methoxymethoxy)-4-(4,4,5,5-tetramethyl-1,3,2-dioxaborolan-2-yl)phenyl]-3-methoxypyridazine (731.38 mg, 1.875 mmol, 2.5 equiv),1-(6-chloropyridazin-3-yl)-N-(1-methylcyclobutyl)pyrrolidin-3-amine (200 mg, 0.750 mmol, 1.00 equiv) in 1,4-dioxane (10 mL) and H₂O (2 mL) was treated with K₃PO₄ (477.41 mg, 2.250 mmol, 3 equiv) and RuPhos Palladacycle Gen.3 (62.7 mg, 0.075 mmol, 0.1 equiv) and RuPhos (34.98 mg, 0.075 mmol, 0.1 equiv) for 3 h at 80 degrees C. under nitrogen atmosphere followed. The mixture was allowed to cool down to room temperature. The reaction was quenched with H₂O at room temperature. The resulting mixture was extracted with EA (2×10 mL). The combined organic layers were washed with brine Solution (1×10 mL), dried over anhydrous Na₂SO₄. After filtration, the filtrate was concentrated under reduced pressure. The residue was purified by silica gel column chromatography, eluted with DCM:MeOH (9:1) to afford 1-{6-[5-fluoro-2-(methoxymethoxy)-4-(6-methoxypyridazin-4-yl)phenyl]pyridazin-3-yl}-N-(1-methylcyclobutyl)pyrrolidin-3-amine (160 mg, 32%) as a solid. LCMS (ES, m/z): 495 [M+H]⁺

### Synthesis of Intermediate B208

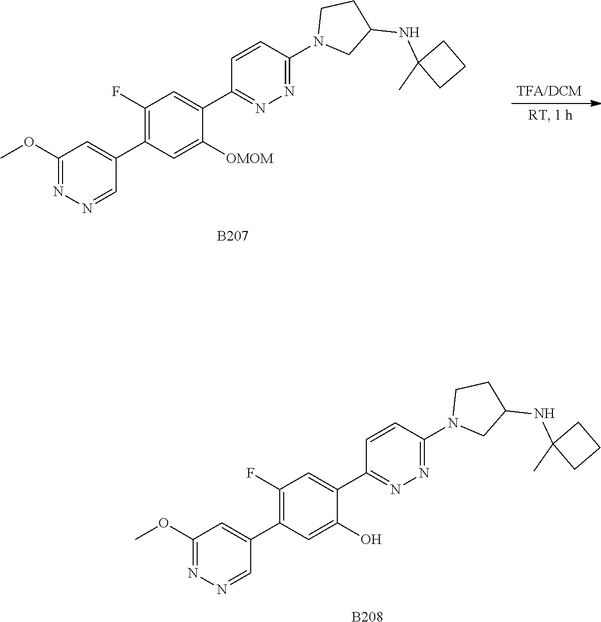

To a stirred solution of 1-{6-[5-fluoro-2-(methoxymethoxy)-4-(6-methoxypyridazin-4-yl)phenyl]pyridazin-3-yl}-N-(1-methylcyclobutyl)pyrrolidin-3-amine (160 mg, 0.324 mmol, 1 equiv) in DCM (2 mL) was added TFA (3 mL) dropwise at room temperature.

The mixture was stirred 1 h at room temperature. The reaction liquid was concentrated under reduced pressure. The crude product was purified by reverse phase flash with the following conditions (Column: XBridge Shield RP18 OBD Column, 30×150 mm, 5 m; Mobile Phase A: water (10 mmol/L NH₄HCO₃), Mobile Phase B: ACN; Flow rate: 60 mL/min; Gradient: 25% B to 65% B in 10 min, 65% B; Wave Length: UV 220 nm; RT₁(min): 8.35) to afford 4-fluoro-5-(6-methoxypyridazin-4-yl)-2-(6-{3-[(1-methylcyclobutyl)amino]pyrrolidin-1-yl}pyridazin-3-yl)phenol (52 mg, 36%) as a solid. LCMS (ES, m/z): 451 [M+H]⁺

### Synthesis of Compounds 158 and 159

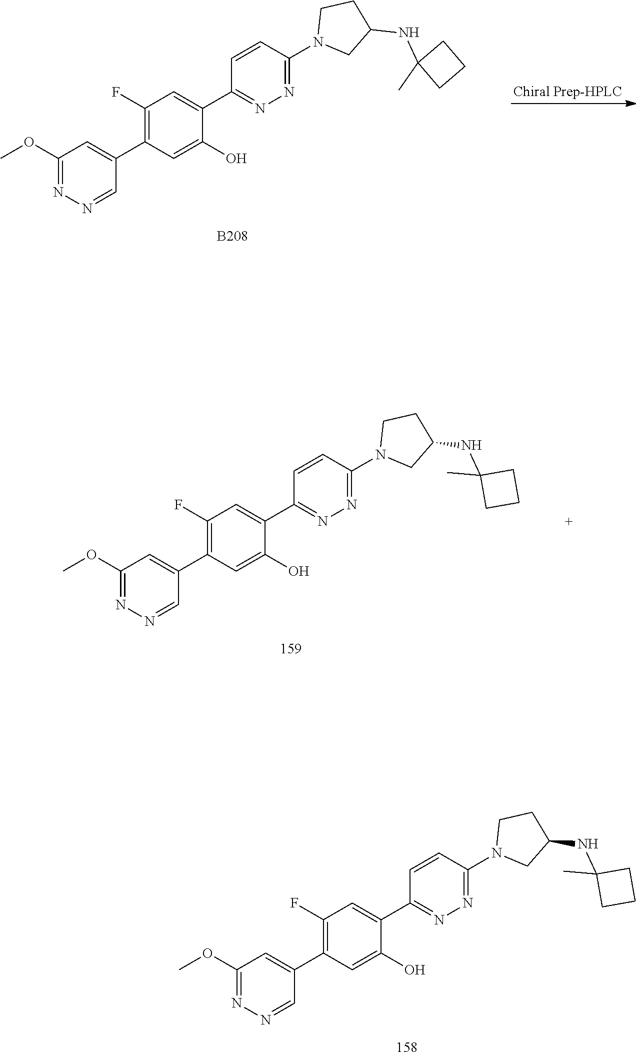 [SMILES: "B208" ; COc1cc(-c2cc(O)c(-c3ccc(N4CCC(NC5(C)CCC5)C4)nn3)cc2F)cnn1 ; COc1cc(-c2cc(O)c(-c3ccc(N4CC[C@H](NC5(C)CCC5)C4)nn3)cc2F)cnn1 ; "159" ; COc1cc(-c2cc(O)c(-c3ccc(N4CC[C@@H](NC5(C)CCC5)C4)nn3)cc2F)cnn1 ; "158"]

The residue was purified by reverse flash chromatography with the following conditions: Column: CHIRAL ART Cellulose-SB, 4.6×100 mm, 3.Oum; Mobile Phase A: MTBE(0.1% DEA): MeOH=50: 50; Flow rate: 1 mL/min; Gradient: 0% B to 0% B; Injection Volume: Sul mL to afford first eluting 4-fluoro-5-(6-methoxypyridazin-4-yl)-2-{6-[(3R)-3-[(1-methylcyclobutyl)amino]pyrrolidin-1-yl]pyridazin-3-yl}phenol (8.7 mg, 16.73%) as a yellow solid and second eluting 4-fluoro-5-(6-methoxypyridazin-4-yl)-2-{6-[(3R)-3-[(1-methylcyclobutyl)amino]pyrrolidin-1-yl]pyridazin-3-yl}phenol (11.2 mg, 20%) as a solid. Compound 159: LCMS (ES, m/z): 451 [M+H]^{+ 1}H NMR (400 MHz, DMSO-d₆) δ 13.82 (s, 1H), 9.17 (t, J=1.9 Hz, 1H), 8.31 (d, J=9.9 Hz, 1H), 8.00 (d, J=12.5 Hz, 1H), 7.47-7.42 (m, 1H), 7.30 (d, J=6.9 Hz, 1H), 7.19 (d, J=9.8 Hz, 1H), 4.09 (s, 3H), 3.74-3.67 (m, 2H), 3.48 (q, J=9.6, 8.3 Hz, 2H), 3.20-3.13 (m, 1H), 2.14 (dt, J=12.1, 6.0 Hz, 1H), 1.99-1.88 (m, 2H), 1.79 (qd, J=7.8, 4.4 Hz, 3H), 1.78-1.55 (m, 2H), 1.26 (s, 3H). Compound 158: LCMS (ES, m/z): 451 [M+H]^{+ 1}HNMR (400 MHz, DMSO-d₆) δ 13.82 (s, 1H), 9.16 (t, J=1.9 Hz, 1H), 8.31 (d, J=9.9 Hz, 1H), 8.00 (d, J=12.5 Hz, 1H), 7.47-7.42 (m, 1H), 7.30 (d, J=6.9 Hz, 1H), 7.19 (d, J=9.8 Hz, 1H), 4.09 (s, 3H), 3.75-3.62 (m, 2H), 3.56-3.42 (m, 2H), 3.16 (t, J=8.7 Hz, 1H), 2.19-2.09 (m, 1H), 2.00-1.88 (m, 2H), 1.86-1.74 (m, 3H), 1.77-1.58 (m, 2H), 1.26 (s, 3H).

### Example 79: Synthesis of Compounds 287 and 289

### Synthesis of Intermediate B209

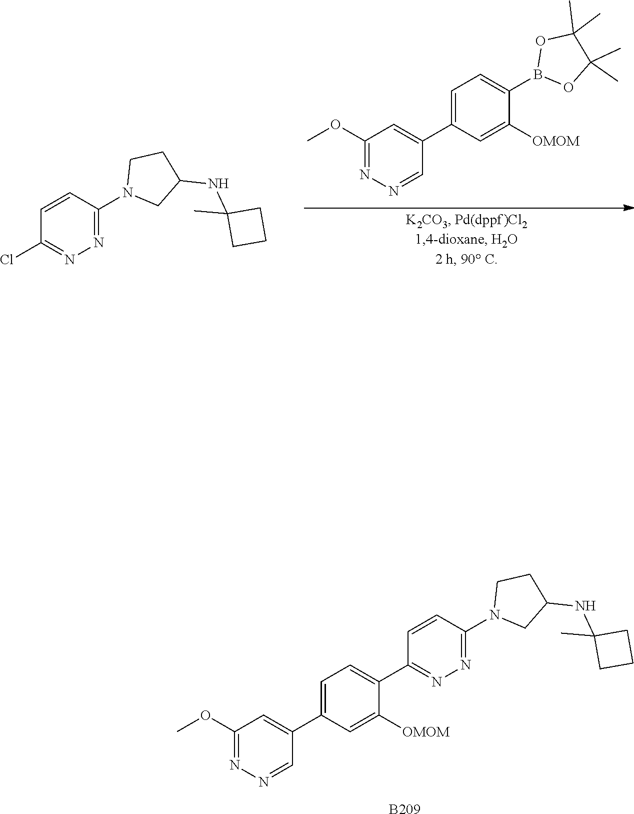

To a solution of 1-(6-chloropyridazin-3-yl)-N-(1-methylcyclobutyl)pyrrolidin-3-amine (240 mg, 0.900 mmol, 1 equiv) and 3-methoxy-5-[3-(methoxymethoxy)-4-(4,4,5,5-tetramethyl-1,3,2-dioxaborolan-2-yl)phenyl]pyridazine (502.32 mg, 1.350 mmol, 1.5 equiv) in 1,4-dioxane (2 mL) and H₂O (0.4 mL) were added K₂CO₃ (373.01 mg, 2.700 mmol, 3 equiv) and Pd(dppf)Cl₂ (65.83 mg, 0.090 mmol, 0.1 equiv). After stirring for 2 h at 90° C. under a nitrogen atmosphere, the resulting mixture was concentrated under reduced pressure. The residue was purified by silica gel column chromatography, eluted with CH₂Cl₂/MeOH (10:1) to afford 1-{6-[2-(methoxymethoxy)-4-(6-methoxypyridazin-4-yl)phenyl]pyridazin-3-yl}-N-(1-methylcyclobutyl)pyrrolidin-3-amine (110 mg, 26%) as a solid.

### Synthesis of Compounds 287 and 289

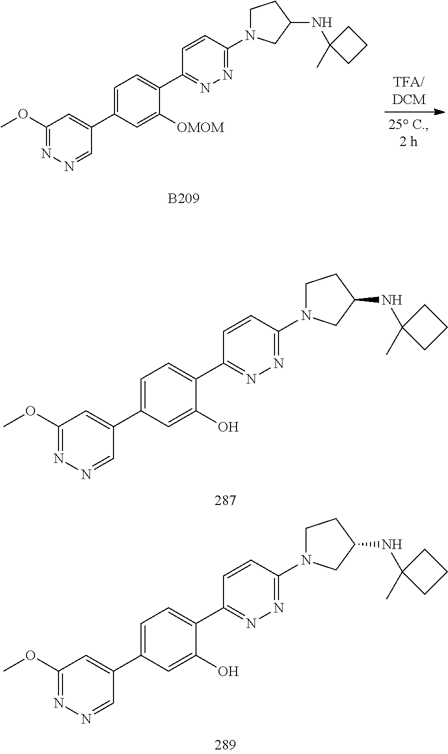

To a solution of 1-{6-[2-(methoxymethoxy)-4-(6-methoxypyridazin-4-yl)phenyl]pyridazin-3-yl}-N-(1-methylcyclobutyl)pyrrolidin-3-amine (100 mg, 0.210 mmol, 1 equiv) in DCM (3 mL) was added TFA (1 mL, 13.463 mmol, 64.16 equiv). After stirring for 2 h at 25° C. under a nitrogen atmosphere, the resulting mixture was concentrated under reduced pressure. The crude product was purified by Chiral-Prep-HPLC with the following conditions (2 #SHIMADZU (HPLC-01)): Column, Kinetex EVO prep C18, 30*150, 5 um; mobile phase, Water (10 mmol/L NH₄HCO₃) and ACN (20% ACN up to 50% in 8 min) to afford the crude product; The crude product was purified by Chiral-Prep-HPLC with the following conditions (2 #SHIMADZU (HPLC-01)): Column, CHIRAL ART Cellulose-SB, 2*25 cm, 5 um; mobile phase, MtBE(0.1% DEA) and MeOH- (hold 50% MeOH- in 15 min) to afford (R)-5-(6-methoxypyridazin-4-yl)-2-(6-(3-((1-methylcyclobutyl)amino)pyrrolidin-1-yl)pyridazin-3-yl)phenol (7.3 mg, 8.04%) as a light yellow solid. and (S)-5-(6-methoxypyridazin-4-yl)-2-(6-(3-((1-methylcyclobutyl)amino)pyrrolidin-1-yl)pyridazin-3-yl)phenol (8.4 mg, 9%) as a solid.

Compound 287: LCMS:(ES, m/z):432.91HNMR: (400 MHz, DMSO-d₆) δ 14.11 (s, 1H), 9.35 (d, J=1.9 Hz, 1H), 8.29 (d, J=9.8 Hz, 1H), 8.04 (d, J=8.3 Hz, 1H), 7.55 (d, J=1.9 Hz, 1H), 7.52-7.42 (m, 2H), 7.17 (d, J=9.8 Hz, 1H), 4.08 (s, 3H), 3.81-3.61 (m, 2H), 3.56-3.38 (m, 2H), 3.15 (dd, J=10.4, 6.7 Hz, 1H), 2.15 (d, J=5.7 Hz, 1H), 2.00-1.87 (m, 2H), 1.79 (td, J=8.0, 4.1 Hz, 3H), 1.65 (d, J=12.8 Hz, 2H), 1.26 (s, 3H). Compound 289: LCMS:(ES, m/z):432.9 ¹H NMR: (400 MHz, 1,4-Dioxane-d₈) δ 15.15 (s, 1H), 10.39 (d, J=1.9 Hz, 1H), 9.34 (d, J=9.8 Hz, 1H), 9.09 (d, J=8.3 Hz, 1H), 8.60 (d, J=1.9 Hz, 1H), 8.57-8.47 (m, 2H), 8.22 (d, J=9.8 Hz, 1H), 5.13 (s, 3H), 4.78 (d, J=7.9 Hz, 1H), 4.72 (s, 1H), 4.63-4.44 (m, 2H), 4.20 (dd, J=10.4, 6.7 Hz, 1H), 3.20 (d, J=5.8 Hz, 1H), 3.05-2.92 (m, 2H), 2.83 (td, J=7.8, 4.0 Hz, 3H), 2.70 (d, J=12.8 Hz, 2H), 2.31 (s, 3H).

### Example 80: Synthesis of Compound 291

### Synthesis of Intermediate B210

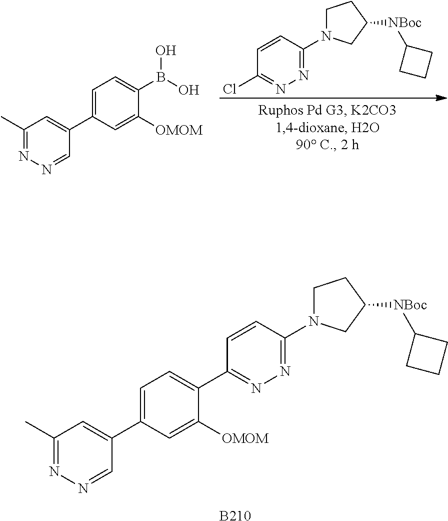

To a solution of 2-(methoxymethoxy)-4-(6-methylpyridazin-4-yl)phenylboronic acid (50 mg, 0.182 mmol, 1 equiv) and tert-butyl N-[(3S)-1-(6-chloropyridazin-3-yl)pyrrolidin-3-yl]-N-cyclobutylcarbamate (64.37 mg, 0.182 mmol, 1 equiv) in 1,4-dioxane (1 mL) and H₂O (0.25 mL, 13.877 mmol, 76.07 equiv) were added K₂CO₃ (75.64 mg, 0.546 mmol, 3 equiv) and RuPhos Palladacycle Gen.3 (8.51 mg, 0.018 mmol, 0.1 equiv). After stirring for 2 h at 90° C. under a nitrogen atmosphere, the resulting mixture was concentrated under reduced pressure. The residue was purified by Prep-TLC/silica gel column chromatography, eluted with CH₂Cl₂/MeOH (10:1) to afford tert-butyl N-cyclobutyl-N-[(3S)-1-{6-[2-(methoxymethoxy)-4-(6-methylpyridazin-4-yl)phenyl]pyridazin-3-yl}pyrrolidin-3-yl]carbamate (30 mg, 30%) as a solid. LCMS:(ES, m/z): 403 [M+H]⁺

### Synthesis of Compound 291

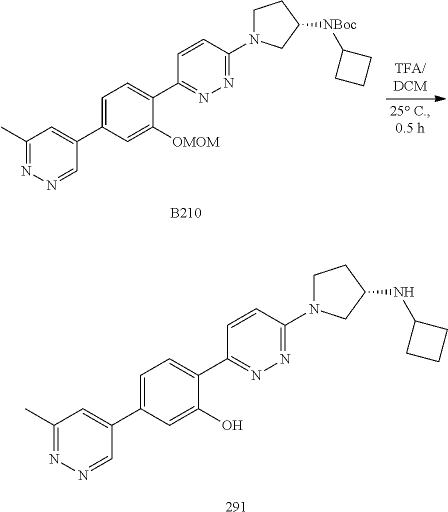

To a solution of tert-butyl N-cyclobutyl-N-[(3S)-1-{6-[2-(methoxymethoxy)-4-(6-methylpyridazin-4-yl)phenyl]pyridazin-3-yl}pyrrolidin-3-yl]carbamate (30 mg, 0.055 mmol, 1 equiv) in DCM (1 mL) was added TFA (0.3 mL). After stirring for 0.5 h at 25° C. under a nitrogen atmosphere, the resulting mixture was concentrated under reduced pressure. The crude product was purified by Chiral-Prep-HPLC with the following conditions (2 #SHIMADZU (HPLC-01)): Column, XBridge Shield RP18 OBD Column, 30*150 mm, 5 pm; mobile phase, Water (10 mmol/L NH₄HCO₃) and ACN (15% ACN up to 45% in 8 min) to afford 2-{6-[(3S)-3-(cyclobutylamino)pyrrolidin-1-yl]pyridazin-3-yl}-5-(6-methylpyridazin-4-yl)phenol (3.7 mg, 17%) as a solid. LCMS (ES, m/z): 402.9 [M+H]^{+ 1}H NMR (400 MHz, DMSO-d₆) δ 14.12 (s, 1H), 9.50 (d, J=2.3 Hz, 1H), 8.31 (d, J=9.8 Hz, 1H), 8.06 (d, J=8.3 Hz, 1H), 7.95 (d, J=2.3 Hz, 1H), 7.52-7.44 (m, 2H), 7.18 (d, J=9.7 Hz, 1H), 3.64 (dt, J=14.5, 7.2 Hz, 2H), 3.52 (t, J 8.0 Hz, 1H), 3.39 (t, J=5.6 Hz, 1H), 3.25 (t, J=7.5 Hz, 1H), 2.69 (s, 3H), 2.19-2.04 (m, 4H), 1.83 (dq, J=12.9, 6.6 Hz, 1H), 1.78-1.63 (m, 2H), 1.63-1.50 (m, 2H).

### Example 81: Synthesis of Compound 292

### Synthesis of Intermediate B211

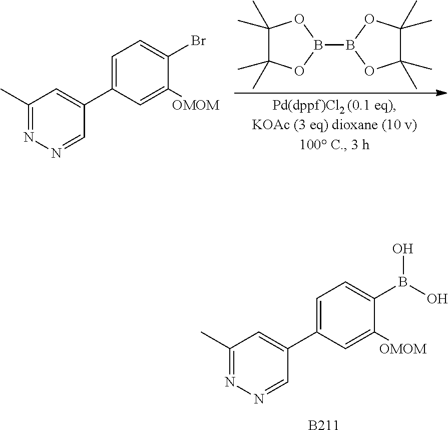

A solution of 5-[4-bromo-3-(methoxymethoxy)phenyl]-3-methylpyridazine (100 mg, 0.323 mmol, 1.00 equiv),Pd(dppf)Cl₂(26 mg, 0.032 mmol, 0.1 equiv), KOAc (95 mg, 0.969 mmol, 3 equiv) in 1,4-dioxane (10 mL) was treated with bis(pinacolato)diboron (123 mg, 0.485 mmol, 1.5 equiv) for 3 h at 100° C. The mixture was allowed to cool down to room temperature. The resulting mixture was filtered and the filter cake was washed with DCM (3×5 mL). The filtrate was concentrated under reduced pressure. The residue was purified by Prep-TLC (PE/EA 1:1) to afford 2-(methoxymethoxy)-4-(6-methylpyridazin-4-yl)phenylboronic acid (50 mg, 56.40%) as a solid. LCMS:(ES, m/z):275 [M+H]⁺

### Synthesis of Intermediate B212

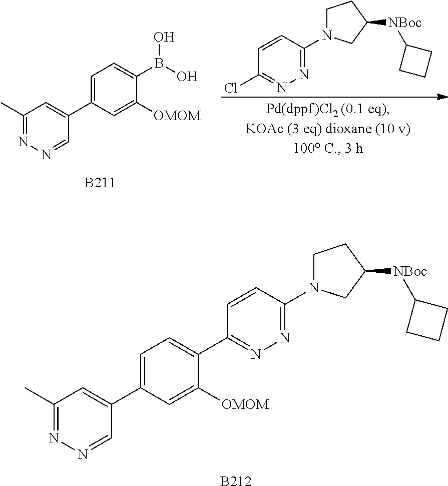

A mixture of 2-(methoxymethoxy)-4-(6-methylpyridazin-4-yl)phenylboronic acid (65 mg, 0.146 mmol, 1 equiv) and tert-butyl N-[(3R)-1-(6-chloropyridazin-3-yl)pyrrolidin-3-yl]-N-cyclobutylcarbamate (51.5 mg, 0.146 mmol, 1 equiv), RuPhos Palladacycle Gen.3 (12 mg, 0.015 mmol, 0.1 equiv), K₂CO₃ (60 mg, 0.438 mmol, 3 equiv) in 1,4-dioxane (1 mL), H₂O (0.25 mL) was stirred for 2 h at 100° C. under nitrogen atmosphere. The mixture was allowed to cool down to room temperature. The resulting mixture was filtered and the filter cake was washed with DCM (3×2 mL). The filtrate was concentrated under reduced pressure. The residue was purified by silica gel column chromatography, eluted with PE/EA (1:1) to afford tert-butyl N-cyclobutyl-N-[(3R)-1-{6-[2-(methoxymethoxy)-4-(6-methylpyridazin-4-yl)phenyl]pyridazin-3-yl}pyrrolidin-3-yl]carbamate (65 mg, 81%) as a solid. LCMS:(ES, m/z):547 [M+H]⁺

### Synthesis of Compound 292

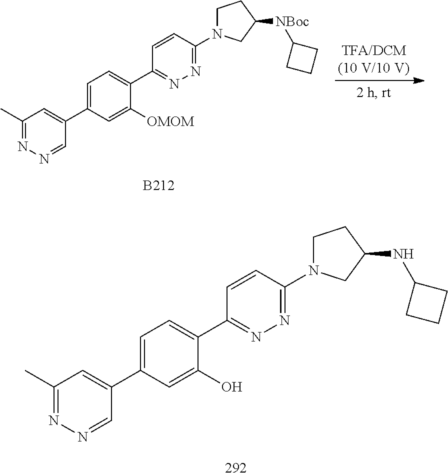

A mixture of (3R)—N-cyclobutyl-1-{6-[2-(methoxymethoxy)-4-(6-methylpyridazin-4-yl)phenyl]pyridazin-3-yl}pyrrolidin-3-amine (64 mg, 0.143 mmol, 1 equiv) and TFA (0.75 mL, 0.007 mmol, 0.05 equiv) in DCM(0.75 mL) was stirred for 2 h at 30° C. The resulting mixture was concentrated under reduced pressure. The crude product was purified by Prep-HPLC with the following conditions (Column: CHIRAL ART Cellulose-SB, 4.6*100 mm, 3.Oum; Mobile Phase A: MtBE(0.1% DEA): MeOH=50: 50; Flow rate: 1 mL/min; Gradient: 0% B to 0% B; Injection Volume: Sul mL, Column: CHIRAL ART Cellulose-SB, 4.6*100 mm, 3.Oum; Mobile Phase A: MtBE(0.1% DEA): MeOH=50: 50; Flow rate: 1 mL/min; Gradient: 0% B to 0% B; Injection Volume: Sul mL) to afford 2-{6-[(3R)-3-(cyclobutylamino)pyrrolidin-1-yl]pyridazin-3-yl}-5-(6-methylpyridazin-4-yl)phenol (10.6 mg, 18%) as a solid. LCMS:(ES, m/z):403 [M+H]¹H NMR: (400 MHz, DMSO-d₆) δ 14.12 (s, 1H), 9.50 (d, J=2.3 Hz, 1H), 8.31 (d, J=9.8 Hz, 1H), 8.06 (d, J=8.3 Hz, 1H), 7.95 (d, J=2.2 Hz, 1H), 7.53-7.44 (m, 2H), 7.18 (d, J=9.8 Hz, 1H), 3.69-3.58 (m, 2H), 3.51 (d, J=8.7 Hz, 1H), 3.39 (d, J=5.6 Hz, 1H), 3.29-3.21 (m, 2H), 2.69 (s, 3H), 2.11 (dq, J=24.1, 6.3, 5.6 Hz, 3H), 2.07 (s, 1H), 1.89-1.77 (m, 2H), 1.64-1.50 (m, 2H).

### Example 82: Synthesis of Compound 293

### Synthesis of Intermediate B213

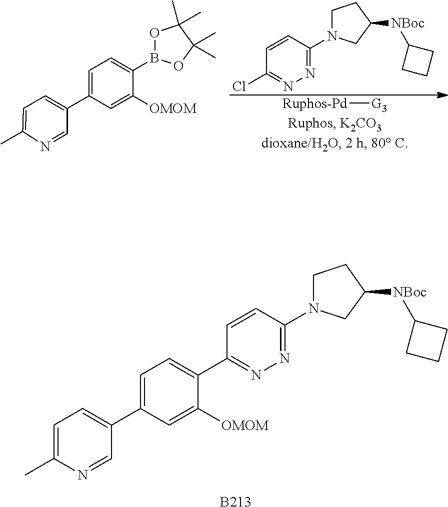

Into a 10 mL vial were added 5-[3-(methoxymethoxy)-4-(4,4,5,5-tetramethyl-1,3,2-dioxaborolan-2-yl)phenyl]-2-methylpyridine (196.32 mg, 0.552 mmol, 1.5 equiv), tert-butyl N-[(3R)-1-(6-chloropyridazin-3-yl)pyrrolidin-3-yl]-N-cyclobutylcarbamate (130 mg, 0.368 mmol, 1 equiv), Ruphos (17.19 mg, 0.037 mmol, 0.1 equiv), RuPhos Palladacycle Gen.3 (30.81 mg, 0.037 mmol, 0.1 equiv), K₂CO₃ (152.75 mg, 1.104 mmol, 3 equiv) and dioxane (0.6 mL)/H₂O (3 mL) at room temperature. The final reaction mixture was stirred for 2 h at 80° C. The residue was purified by silica gel column chromatography, eluted with PE/EA (1/3) to afford tert-butyl N-cyclobutyl-N-[(3R)-1-{6-[2-(methoxymethoxy)-4-(6-methylpyridin-3-yl)phenyl]pyridazin-3-yl}pyrrolidin-3-yl]carbamate (170 mg, 85%) as an solid.

### Synthesis of Compound 293

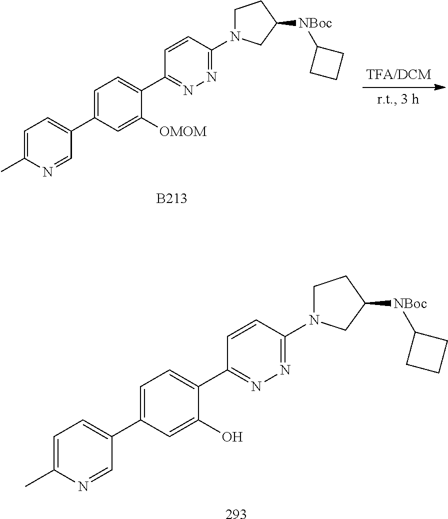

A solution of tert-butyl N-cyclobutyl-N-[(3R)-1-{6-[2-(methoxymethoxy)-4-(6-methylpyridin-3-yl)phenyl]pyridazin-3-yl}pyrrolidin-3-yl]carbamate (100 mg, 0.183 mmol, 1 equiv) in DCM (3 mL)was treated with TFA (1 mL) for 3 h at room temperature. The resulting mixture was concentrated under vacuum. The crude product was purified by Chiral-Prep-HPLC with the following conditions (2 #SHIMADZU (HPLC-01)): Column, XBridge Shield RP18 OBD Column, 30*150 mm, 5 μm; mobile phase, Water (10 mmol/L NH₄HCO₃) and ACN (20% ACN up to 65% in 8 min) to afford 2-{6-[(3R)-3-(cyclobutylamino)pyrrolidin-1-yl]pyridazin-3-yl}-5-(6-methylpyridin-3-yl)phenol (20.2 mg, 27%) as a solid. LCMS:(ES, m/z):402 ¹HNMR: (400 MHz, DMSO-d₆) δ 13.97 (s, 1H), 8.80 (d, J=2.5 Hz, 1H), 8.25 (d, J=9.8 Hz, 1H), 8.01 (dd, J=8.1, 2.5 Hz, 1H), 7.98-7.93 (m, 1H), 7.34 (d, J=8.1 Hz, 1H), 7.26 (h, J=1.9 Hz, 2H), 7.15 (d, J=9.7 Hz, 1H), 3.62 (tt, J=13.4, 6.5 Hz, 2H), 3.49 (dt, J=10.4, 7.1 Hz, 1H), 3.38 (p, J=5.7 Hz, 1H), 3.25 (p, J=7.9 Hz, 2H), 2.52 (s, 3H), 2.23-2.02 (m, 4H), 1.82 (ddd, J=13.9, 12.3, 6.8 Hz, 1H), 1.76-1.62 (m, 2H), 1.62-1.51 (m, 2H).

### Example 83: Synthesis of Compound 294

### Synthesis of Intermediate B214

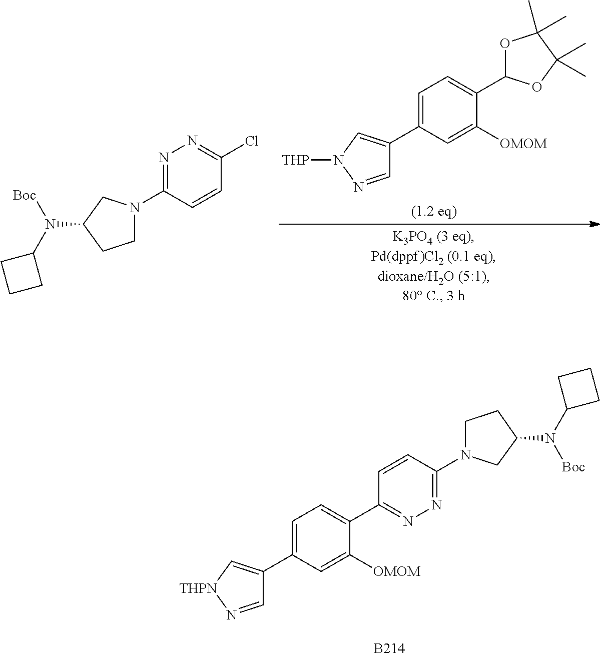

To a solution of tert-butyl N-[(3S)-1-(6-chloropyridazin-3-yl)pyrrolidin-3-yl]-N-cyclobutylcarbamate (110 mg, 0.312 mmol, 1 equiv) and 4-[3-(methoxymethoxy)-4-(4,4,5,5-tetramethyl-1,3,2-dioxaborolan-2-yl)phenyl]-1-(oxan-2-yl)pyrazole (154.99 mg, 0.374 mmol, 1.2 equiv) in H₂O (0.5 mL) and dioxane (2.5 mL) were added K₃PO₄ (198.51 mg, 0.936 mmol, 3 equiv) and Pd(dppf)Cl₂·CH₂Cl₂ (17.22 mg, 0.031 mmol, 0.1 equiv). After stirring for 3 h at 80° C. under a nitrogen atmosphere, the resulting mixture was concentrated under reduced pressure. The residue was purified by Prep-silica gel column chromatography, eluted with PE/EA (1:1) to afford tert-butyl N-cyclobutyl-N-[(3S)-1-{6-[2-(methoxymethoxy)-4-[1-(oxan-2-yl)pyrazol-4-yl]phenyl]pyridazin-3-yl}pyrrolidin-3-yl]carbamate (150 mg, 80%) as a solid. LCMS: (ES, m/z): 605 [M+H]⁺

### Synthesis of Intermediate B215

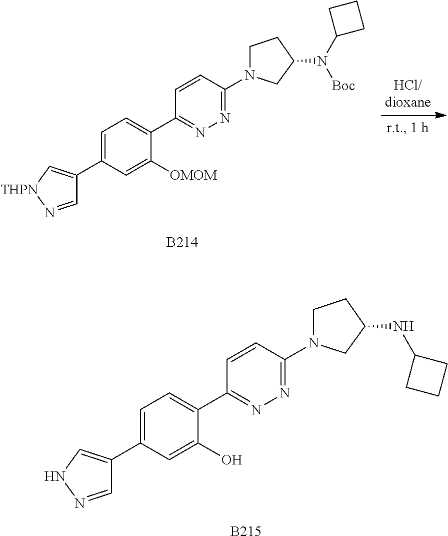

1. A solution of tert-butyl N-cyclobutyl-N-[(3S)-1-{6-[2-(methoxymethoxy)-4-[1-(oxan-2-yl)pyrazol-4-yl]phenyl]pyridazin-3-yl}pyrrolidin-3-yl]carbamate (150 mg, 0.248 mmol, 1 equiv) and HCl(gas) in 1,4-dioxane (3 mL, 98.738 mmol, 398.08 equiv) was stirred for 1 h at room temperature. The resulting mixture was concentrated under reduced pressure. This resulted in 2-{6-[(3S)-3-(cyclobutylamino)pyrrolidin-1-yl]pyridazin-3-yl}-5-(1H-pyrazol-4-yl)phenol (90 mg, 96%) as a solid. LCMS: (ES, m/z): 377 [M+H]⁺

### Synthesis of Compound 294

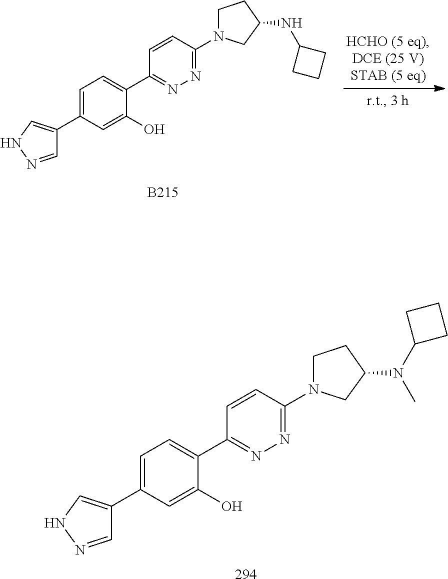 [SMILES: "B215" ; Oc1cc(-c2cn[nH]c2)ccc1-c1ccc(N2CC[C@H](NC3CCC3)C2)nn1 ; CN(C1CCC1)[C@H]1CCN(c2ccc(-c3ccc(-c4cn[nH]c4)cc3O)nn2)C1 ; "294"]

A solution of 2-{6-[(3S)-3-(cyclobutylamino)pyrrolidin-1-yl]pyridazin-3-yl}-5-(1H-pyrazol-4-yl)phenol (90 mg, 0.239 mmol, 1 equiv) in DCE (2.5 mL) was treated with HCHO (71.78 mg, 2.390 mmol, 10 equiv) for 3 h at room temperature followed by the addition of STAB (253.34 mg, 1.195 mmol, 5 equiv) dropwise at room temperature. The reaction was quenched by the addition of Water (3 mL) at room temperature. The resulting mixture was extracted with EtOAc (3×3 mL). The combined organic layers were washed with sat. salt water (2×3 mL), dried over anhydrous Na₂SO₄. After filtration, the filtrate was concentrated under reduced pressure. The residue/crude product was purified by reverse phase flash with the following conditions (Column: Kinetex EVO C18 Column, 30*150, 5 um; Mobile Phase A: Water (10 mmol/L NH₄HCO₃), Mobile Phase B: ACN; Flow rate: 60 mL/min; Gradient: 20% B to 50% B in 10 min, 50% B; Wave Length: UV 220 nm; RT1(min): 7.35; Number Of Runs: 0) to afford 2-{6-[(3S)-3-[cyclobutyl(methyl)amino]pyrrolidin-1-yl]pyridazin-3-yl}-5-(1H-pyrazol-4-yl)phenol (14.7 mg, 16%) as a solid. LCMS: (ES, m/z): 391 [M+H] ¹H NMR: (400 MHz, DMSO-d₆) δ 13.89 (s, 1H), 12.97 (s, 1H), 8.23 (s, 1H), 8.22 (d, J=9.9 Hz, 1H), 7.98 (s, 1H), 7.84 (d, J=8.3 Hz, 1H), 7.22-7.14 (m, 3H), 3.70 (s, 2H), 3.43 (dd, J=10.2, 7.0 Hz, 1H), 3.33-3.23 (m, 1H), 3.14 (p, J=7.5 Hz, 1H), 3.07-2.96 (m, 1H), 2.17-2.11 (m, 1H), 2.10 (s, 3H), 1.99 (dd, J=7.1, 3.3 Hz, 2H), 1.95-1.83 (m, 3H), 1.64-1.51 (m, 2H).

### Example 84: Synthesis of Compound 296

### Synthesis of Intermediate B216

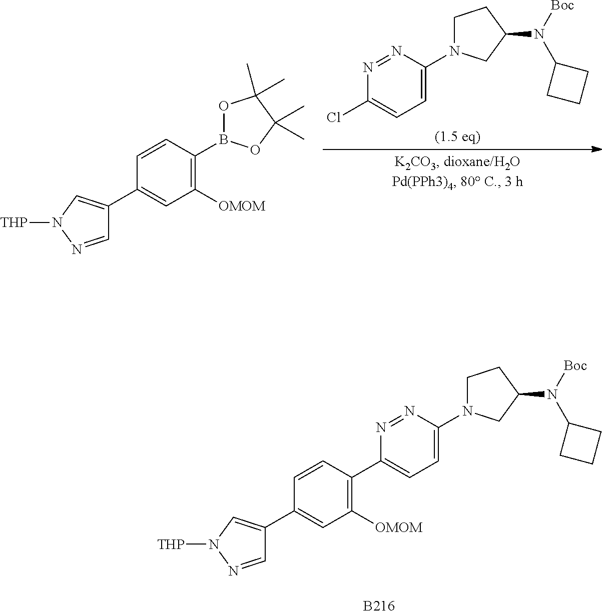

A solution of 4-[3-(methoxymethoxy)-4-(4,4,5,5-tetramethyl-1,3,2-dioxaborolan-2-yl)phenyl]-1-(oxan-2-yl)pyrazole (220 mg, 0.531 mmol, 1 equiv) and tert-butyl N-[(3R)-1-(6-chloropyridazin-3-yl) pyrrolidin-3-yl]-N-cyclobutylcarbamate (149.9 mg, 0.425 mmol, 0.8 equiv) and K₂CO₃ (220.16 mg, 1.593 mmol, 3 equiv) and Pd(PPh₃)₄(61.36 mg, 0.053 mmol, 0.1 equiv) in 1,4-dioxane/H₂O=5:1 was stirred for overnight at 80° C. under nitrogen atmosphere.

The resulting mixture was concentrated under reduced pressure. The residue was purified by silica gel column chromatography, eluted with PE/EA (1:1) to afford tert-butyl N-cyclobutyl-N-[(3R)-1-{6-[2-(methoxymethoxy)-4-[1-(oxan-2-yl) pyrazol-4-yl]phenyl]pyridazin-3-yl}pyrrolidin-3-yl]carbamate (150 mg, 47%) as a solid.

### Synthesis of Intermediate B217

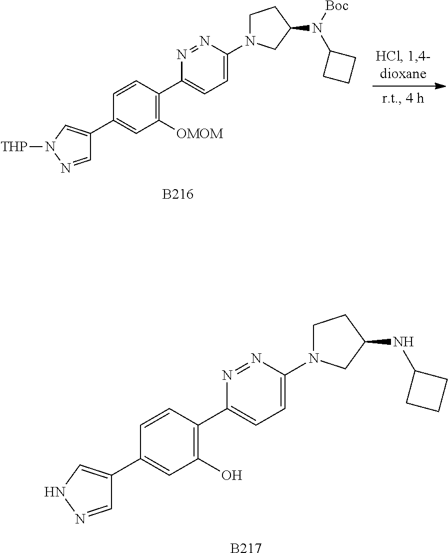

A solution of tert-butyl N-cyclobutyl-N-[(3R)-1-{6-[2-(methoxymethoxy)-4-[1-(oxan-2-yl) pyrazol-4-yl]phenyl]pyridazin-3-yl}pyrrolidin-3-yl]carbamate (150 mg, 0.248 mmol, 1 equiv) in 1,4-dioxane/HCl(5 mL) was stirred for 4 h at room temperature under air atmosphere. The resulting mixture was concentrated under reduced pressure. This resulted in 2-{6-[(3R)-3-(cyclobutylamino)pyrrolidin-1-yl]pyridazin-3-yl}-5-(1H-pyrazol-4-yl)phenol (70 mg, 75%) as a solid.

### Synthesis of Compound 296

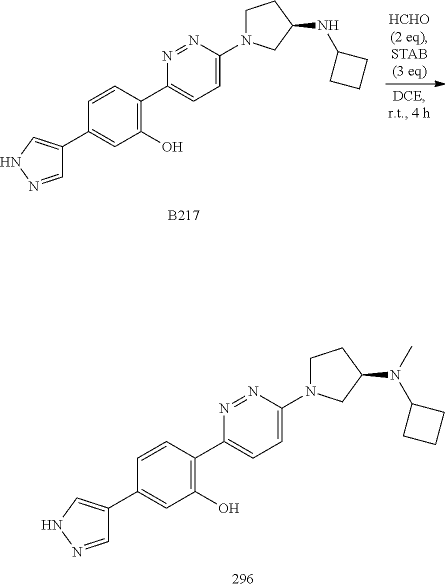 [SMILES: "B217" ; Oc1cc(-c2cn[nH]c2)ccc1-c1ccc(N2CC[C@@H](NC3CCC3)C2)nn1 ; CN(C1CCC1)[C@@H]1CCN(c2ccc(-c3ccc(-c4cn[nH]c4)cc3O)nn2)C1 ; "296"]

A solution of 2-{6-[(3R)-3-(cyclobutylamino)pyrrolidin-1-yl]pyridazin-3-yl}-5-(1H-pyrazol-4-yl)phenol (70 mg, 0.186 mmol, 1 equiv) and HCHO (11.17 mg, 0.372 mmol, 2 equiv) and STAB (118.22 mg, 0.558 mmol, 3 equiv) in DCE(5 mL) was stirred for 4 h at room temperature under nitrogen atmosphere. Column: Kinetex EVO C18 Column, 30*150, 5 um; Mobile Phase A: Water (10 mmol/L NH₄HCO₃), Mobile Phase B: ACN; Flow rate: 60 mL/min; Gradient: 15% B to 55% B in 8 min, 55% B; Wave Length: UV 220 nm; RT1(min): 6.62; Number Of Runs: to afford 2-{6-[(3R)-3-[cyclobutyl(methyl)amino]pyrrolidin-1-yl]pyridazin-3-yl}-5-(1H-pyrazol-4-yl)phenol (6.9 mg, 10%) as a solid. LCMS (ES, m/z): 391 [M+H]⁺

### Example 85: Synthesis of Compound 297

### Synthesis of Intermediate B218

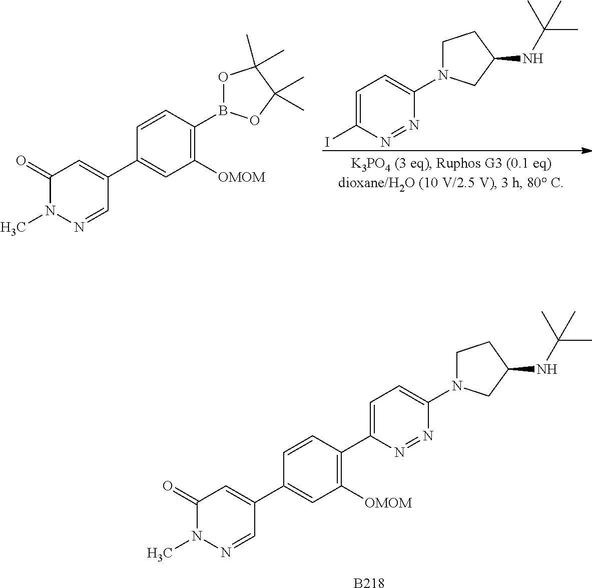

A mixture of 5-[3-(methoxymethoxy)-4-(4,4,5,5-tetramethyl-1,3,2-dioxaborolan-2-yl)phenyl]-2-methylpyridazin-3-one (100 mg, 0.269 mmol, 1.5 equiv) and (3R)—N-tert-butyl-1-(6-iodopyridazin-3-yl)pyrrolidin-3-amine (62 mg, 0.179 mmol, 1 equiv), K₃PO₄ (114 mg, 0.538 mmol, 3 equiv), RuPhos Palladacycle Gen.3 (41.61 mg, 0.050 mmol, 0.1 equiv) in 1,4-dioxane (1 mL, 0.011 mmol), H₂O (0.25 mL, 13.877 mmol) was stirred for 3 h at 80° C. under nitrogen atmosphere. The mixture was allowed to cool down to room temperature. The resulting mixture was filtered and the filter cake was washed with DCM (3×5 mL). The filtrate was concentrated under reduced pressure. The residue was purified by silica gel column chromatography, eluted with PE/EA (1:1) to afford 5-(4-{6-[(3R)-3-(tert-butylamino)pyrrolidin-1-yl]pyridazin-3-yl}-3-(methoxymethoxy)phenyl)-2-methylpyridazin-3-one (19 mg, 23%) as an oil. LCMS:(ES, m/z):465[M+H]

### Synthesis of Compound 297

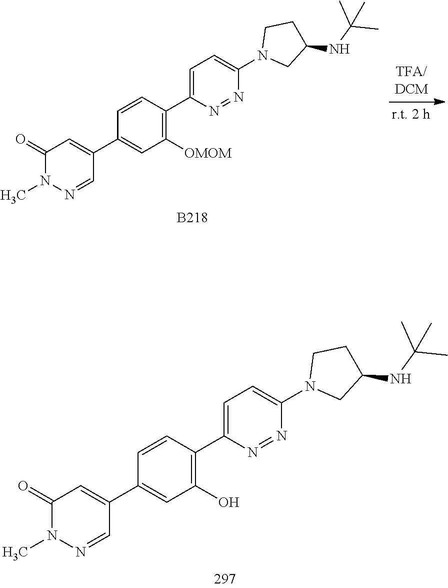

A solution of 5-(4-{6-[(3R)-3-(tert-butylamino)pyrrolidin-1-yl]pyridazin-3-yl}-3-(methoxymethoxy)phenyl)-2-methylpyridazin-3-one (19 mg, 0.041 mmol, 1 equiv) and TFA (0.2 mL, 2.699 mmol) in DCM (0.2 mL) was stirred for 2 h at room temperature under. The mixture was allowed to cool down to room temperature. The resulting mixture was concentrated under reduced pressure. The crude product) was purified by Prep-HPLC with the following conditions (Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient: 15% B to 50% B in 8 min, 50% B; Wave Length: UV 220 nm; RT1(min): 6.58) to afford 5-(4-{6-[(3R)-3-(tert-butylamino)pyrrolidin-1-yl]pyridazin-3-yl}-3-hydroxyphenyl)-2-methylpyridazin-3-one (1.9 mg, 11%) as a solid. LCMS:(ES, m/z):421[M+H] ¹H NMR:(ES, m/z): (400 MHz, Methanol-d₄) δ 8.22 (d, J=2.3 Hz, 1H), 8.07 (d, J=9.8 Hz, 1H), 7.82 (d, J=8.2 Hz, 1H), 7.25-7.17 (m, 2H), 7.13-7.05 (m, 2H), 3.86 (dd, J=10.6, 7.1 Hz, 1H), 3.72-3.61 (m, 5H), 3.51-3.40 (m, 1H), 3.30-3.23 (m, 1H), 2.32 (dd, J=10.1, 6.3 Hz, 1H), 1.98-1.84 (m, 1H), 1.18 (s, 9H).

### Example 86: Synthesis of Compound 298

### Synthesis of Intermediate B219

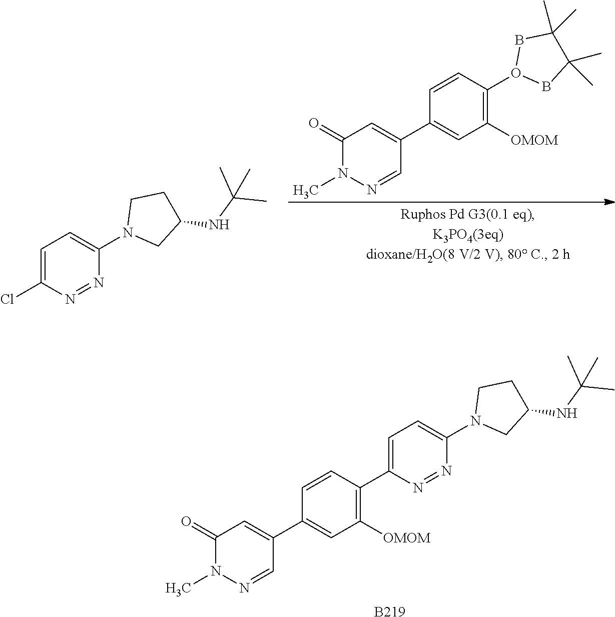

To a solution of (3S)—N-tert-butyl-1-(6-chloropyridazin-3-yl)pyrrolidin-3-amine (50 mg, 0.196 mmol, 1 equiv) and 5-[3-(methoxymethoxy)-4-(4,4,5,5-tetramethyl-1,3,2-dioxaborolan-2-yl)phenyl]-2-methylpyridazin-3-one (110 mg, 0.294 mmol, 1.5 equiv) in water (0.1 mL) and 1,4-dioxane (0.4 mL) were added K₃PO₄ (125 mg, 0.588 mmol, 3 equiv) and RuPhos Palladacycle Gen.3 (16 mg, 0.020 mmol, 0.1 equiv). After stirring for 2 h at 80° C. under a nitrogen atmosphere, the resulting mixture was concentrated under reduced pressure. The residue was purified by silica gel column chromatography, eluted with CH₂Cl₂/MeOH (10:1) to afford 5-(4-{6-[(3S)-3-(tert-butylamino)pyrrolidin-1-yl]pyridazin-3-yl}-3-(methoxymethoxy) phenyl)-2-methylpyridazin-3-one (54 mg, 36%) as a solid. LCMS:(ES, m/z):

### Synthesis of Compound 298

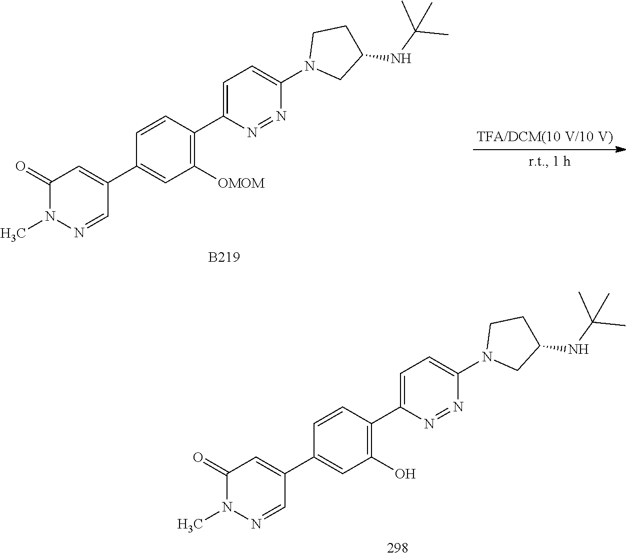

A solution of 5-(4-{6-[(3S)-3-(tert-butylamino)pyrrolidin-1-yl]pyridazin-3-yl}-3-(methoxymethoxy)phenyl)-2-methylpyridazin-3-one (54 mg, 0.116 mmol, 1 equiv) in DCM (0.5 mL). TFA (0.5 mL) was stirred for 1 h at room temperature. The resulting mixture was concentrated under reduced pressure. The crude product was purified by Prep-HPLC with the following conditions (Column: Kinetex EVO C18 Column, 30×150, 5 um; Mobile Phase A: Water (10 mmol/L NH₄HCO₃), Mobile Phase B: ACN; Flow rate: 60 mL/min; Gradient: 10% B to 40% B in 10 min, 40% B; Wave Length: UV 220 nm; RT1(min): 9.85) to afford 5-(4-{6-[(3S)-3-(tert-butylamino)pyrrolidin-1-yl]pyridazin-3-yl}-3-hydroxyphenyl)-2-methylpyridazin-3-one (1.5 mg, 3%) as a solid. LCMS:(ES, m/z):421 ¹H NMR: (400 MHz, Chloroform-d) δ13.76 (s, 1H), 9.87 (s, 1H), 8.04 (d, J=2.3 Hz, 1H), 7.73 (d, J=9.8 Hz, 1H), 7.57 (d, J=8.2 Hz, 1H), 7.20 (s, 1H), 7.07 (q, J=3.0 Hz, 2H), 6.87 (d, J=9.6 Hz, 1H), 4.12 (s, 1H), 3.92 (s, 1H), 3.84 (s, 5H), 3.57 (q, J=8.7 Hz, 1H), 2.57 (s, 2H), 1.53 (s, 9H).

### Example 87: Synthesis of Compounds 308-310

### Synthesis of Intermediate B220

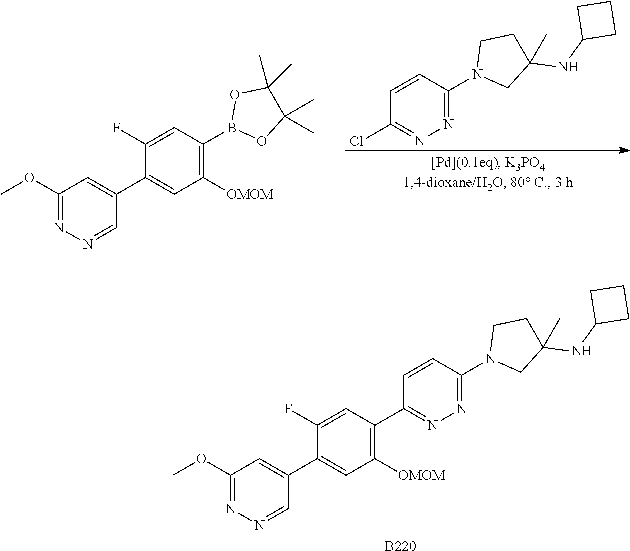

A solution of 1-(6-chloropyridazin-3-yl)-N-cyclobutyl-3-methylpyrrolidin-3-amine (1 g, 3.749 mmol, 1 equiv) and 5-[2-fluoro-5-(methoxymethoxy)-4-(4,4,5,5-tetramethyl-1,3,2-dioxaborolan-2-yl)phenyl]-3-methoxypyridazine (3.66 g, 9.373 mmol, 2.5 equiv), Pd(PPh₃)₄ (0.43 g, 0.375 mmol, 0.1 equiv) and K₃PO₄ (2.39 g, 11.247 mmol, 3 equiv) in 1,4-dioxane/H₂O(10 mL, 5:1) was stirred for overnight at 80° C. under nitrogen atmosphere. The residue was purified by silica gel column chromatography, eluted with CH₂Cl₂/MeOH (10:1) to afford N-cyclobutyl-1-{6-[5-fluoro-2-(methoxymethoxy)-4-(6-methoxypyridazin-4-yl) phenyl]pyridazin-3-yl}-3-methylpyrrolidin-3-amine (450 mg, 24%) as a solid.

### Synthesis of Compound 310

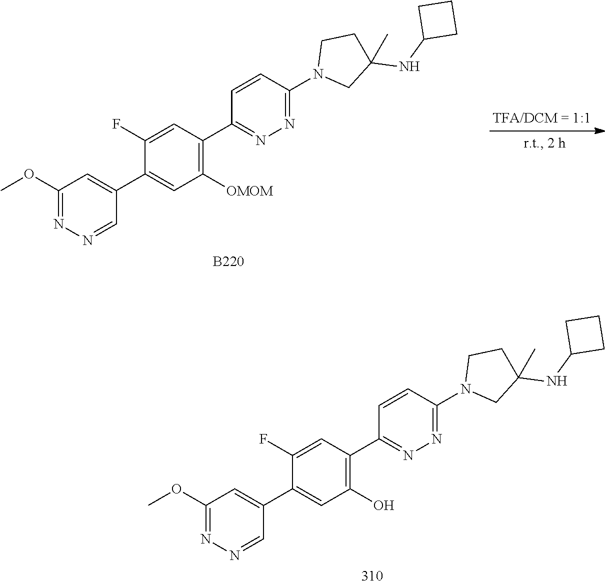

A solution of N-cyclobutyl-1-{6-[5-fluoro-2-(methoxymethoxy)-4-(6-methoxypyridazin-4-yl)phenyl]pyridazin-3-yl}-3-methylpyrrolidin-3-amine (180 mg, 0.364 mmol, 1 equiv) in TFA/DCM=1:1 (10 mL) was stirred for 3 h at room temperature under air atmosphere. Column: Kinetex EVO C18 Column, 30*150, 5 um; Mobile Phase A: Water (10 mmol/L NH₄HCO₃), Mobile Phase B: ACN; Flow rate: 60 mL/min; Gradient: 15% B to 70% B in 10 min, 70% B; Wave Length: UV 220 nm; RT1(min): 7.77; Number Of Runs: to offord 2-{6-[3-(cyclobutylamino)-3-methylpyrrolidin-1-yl]pyridazin-3-yl}-4-fluoro-5-(6-methoxypyridazin-4-yl)phenol (5.6 mg, 3%) as a solid.

### Synthesis of Compounds 308 and 309

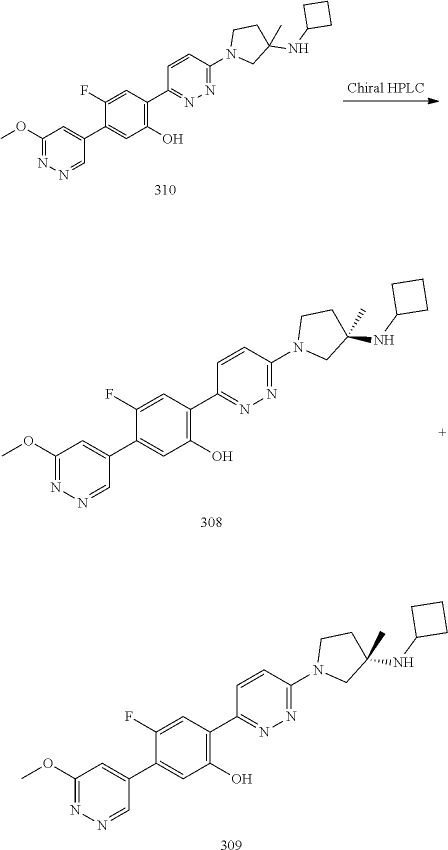 [SMILES: COc1cc(-c2cc(O)c(-c3ccc(N4CCC(C)(NC5CCC5)C4)nn3)cc2F)cnn1 ; "310" ; COc1cc(-c2cc(O)c(-c3ccc(N4CC[C@@](C)(NC5CCC5)C4)nn3)cc2F)cnn1 ; "308" ; COc1cc(-c2cc(O)c(-c3ccc(N4CC[C@](C)(NC5CCC5)C4)nn3)cc2F)cnn1 ; "309"]

The residue was purified by reversed-phase flash chromatography with the following conditions: Column: CHIRALPAK ID-3, 4.6*50 mm, 3 m; Mobile Phase A: MtBE(0.1% DEA): MeOH=50: 50; Flow rate: 1 mL/min; Gradient: 0% B to 0% B; Injection Volume: 5 ul mL to afford 2-{6-[3-(cyclobutylamino)-3-methylpyrrolidin-1-yl]pyridazin-3-yl}-4-fluoro-5-(6-methoxypyridazin-4-yl)phenol (12.3 mg, 15%) as a solid and 2-{6-[3-(cyclobutylamino)-3-methylpyrrolidin-1-yl]pyridazin-3-yl}-4-fluoro-5-(6-methoxypyridazin-4-yl)phenol (8.6 mg, 11%) as a solid. Compound 308: LCMS (ES, m/z): 451 [M+H]^{+ 1}H NMR (400 MHz, DMSO-d₆) δ 13.56 (s, 1H), 9.16 (t, J=1.9 Hz, 1H), 9.09 (s, 2H), 8.38 (d, J=9.8 Hz, 1H), 8.03 (d, J=12.4 Hz, 1H), 7.44 (t, J=1.4 Hz, 1H), 7.29 (m, 2H), 4.10 (s, 3H), 3.94 (s, 1H), 3.67-3.61 (m, 2H), 2.27 (m, 4H), 2.17 (m, 3H), 1.78 (s, 2H), 1.43 (s, 3H) Compound 309: LCMS (ES, m/z): 451 [M+H] ⁺¹H NMR (400 MHz, DMSO-d₆) δ 13.79 (s, 1H), 9.16 (t, J=1.9 Hz, 1H), 8.30 (d, J=9.8 Hz, 1H), 7.99 (d, J=12.5 Hz, 1H), 7.46-7.41 (m, 1H), 7.29 (d, J=6.9 Hz, 1H), 7.16 (d, J=9.7 Hz, 1H), 4.09 (s, 3H), 3.63 (s, 1H), 3.55 (s, 1H), 3.45 (s, 2H), 2.20-2.08 (m, 3H), 2.00 (d, J=12.3 Hz, 1H), 1.83 (q, J=6.1, 5.6 Hz, 1H), 1.72 (s, 3H), 1.53 (m, 2H), 1.22 (s, 3H).

### Example 88: Synthesis of Compound 313

### Synthesis of Intermediate B221

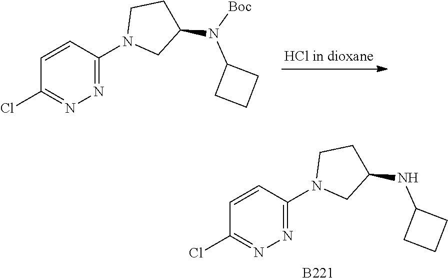 [SMILES: "B221" ; CC(C)(C)OC(=O)N(C1CCC1)[C@@H]1CCN(c2ccc(Cl)nn2)C1 ; Clc1ccc(N2CC[C@@H](NC3CCC3)C2)nn1]

A solution of tert-butyl N-[(3R)-1-(6-chloropyridazin-3-yl)pyrrolidin-3-yl]-N-cyclobutylcarbamate (500 mg, 1.417 mmol, 1 equiv) and HCl(gas) in 1,4-dioxane (3 mL) in MeOH(3 mL) was stirred for 1 h at 25° C. The resulting mixture was concentrated under reduced pressure. This resulted in (3R)-1-(6-chloropyridazin-3-yl)-N-cyclobutylpyrrolidin-3-amine (350 mg, 98%) as an oil. LCMS:(ES, m/z):252.75[M+H]⁺

### Synthesis of Intermediate B222

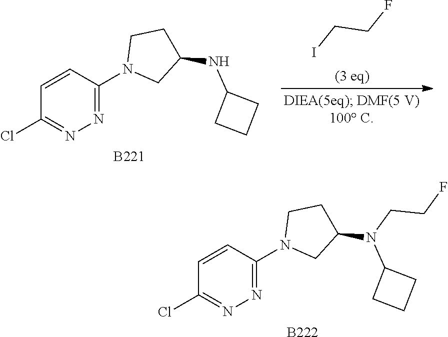

A solution of (3R)-1-(6-chloropyridazin-3-yl)-N-cyclobutylpyrrolidin-3-amine (358 mg, 1.416 mmol, 1 equiv) and ethane, 1-fluoro-2-iodo- (739.19 mg, 4.248 mmol, 3 equiv) and DIEA (1.97 mL, 11.328 mmol, 8 equiv) in DMF (5 mL) was stirred for overnight at 100° C. The mixture was allowed to cool down to room temperature. The reaction was quenched by the addition of water (20 mL) at 25° C. The aqueous layer was extracted with EtOAc (2×20 mL). The residue was purified by silica gel column chromatography, eluted with PE/EA (1:1) to afford (3R)-1-(6-chloropyridazin-3-yl)-N-cyclobutyl-N-(2-fluoroethyl)pyrrolidin-3-amine (245.6 mg, 58%) as a solid. LCMS:(ES, m/z):298.79[M+H]⁺

### Synthesis of Intermediate B223

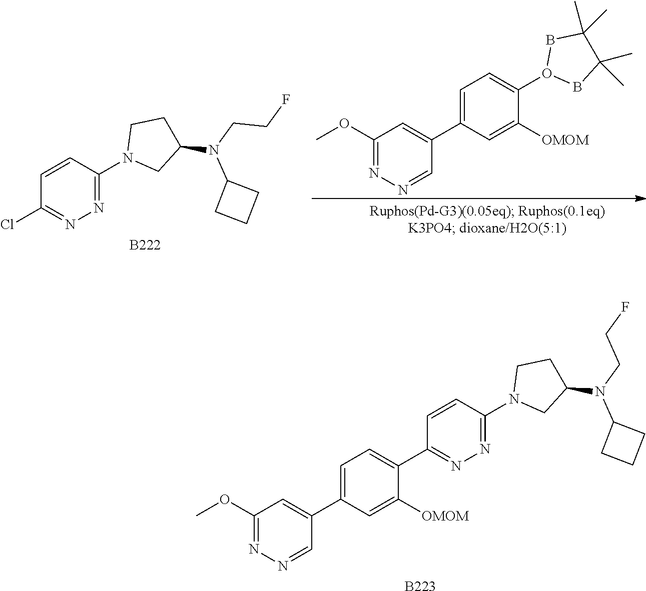

A solution of (3R)-1-(6-chloropyridazin-3-yl)-N-cyclobutyl-N-(2-fluoroethyl)pyrrolidin-3-amine (100 mg, 0.335 mmol, 1 equiv), Ruphos (15.62 mg, 0.034 mmol, 0.1 equiv), RuPhos Palladacycle Gen.3 (14.0 mg, 0.017 mmol, 0.05 equiv) and 3-methoxy-5-[3-(methoxymethoxy)-4-(4,4,5,5-tetramethyl-1,3,2-dioxaborolan-2-yl)phenyl]pyridazine (161.95 mg, 0.436 mmol, 1.3 equiv) in 1,4-dioxane/H₂O(5:1)(6 mL) was stirred for 16 h at 80° C. under nitrogen atmosphere. The residue was purified by silica gel column chromatography, eluted with PE/EA (1:1) to afford (3R)—N-cyclobutyl-N-(2-fluoroethyl)-1-{6-[2-(methoxymethoxy)-4-(6-methoxypyridazin-4-yl)phenyl]pyridazin-3-yl}pyrrolidin-3-amine (156.3 mg, 92%) as an oil. LCMS:(ES, m/z):508.26[M+H]⁺

### Synthesis of Compound 313

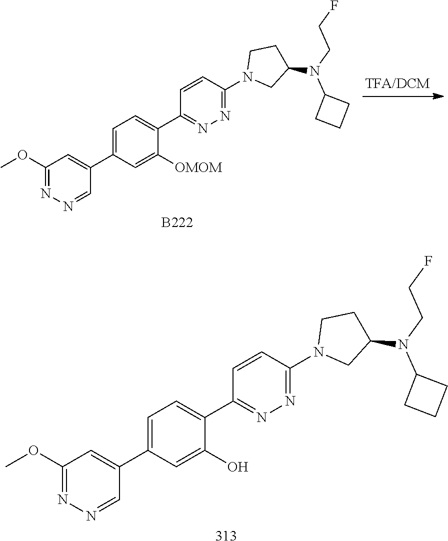

A solution of (3R)—N-cyclobutyl-N-(2-fluoroethyl)-1-{6-[2-(methoxymethoxy)-4-(6-methoxypyridazin-4-yl)phenyl]pyridazin-3-yl}pyrrolidin-3-amine (120 mg, 0.236 mmol, 1 equiv) and TFA (0.2 mL) in DCM(0.5 mL) was stirred for 2 h at 25° C. Desired product could be detected by LCMS. The crude product (100 mg) was purified by Prep-HPLC with the following conditions (Column, Kinetex EVO C18 Column, 30*150.5 um; mobile phase, Water (10 mmol/L NH₄HCO₃) and ACN (20% ACN up to 60% in 10 min)) to afford 2-{6-[(3R)-3-[cyclobutyl(2-fluoroethyl)amino]pyrrolidin-1-yl]pyridazin-3-yl}-5-(6-methoxypyridazin-4-yl)phenol (69 mg, 84%) as a solid. LCMS:(ES, m/z):464.95[M+H]^{+ 1}HNMR: (400 MHz, DMSO-d₆) δ 14.09 (s, 1H), 9.35 (d, J=1.9 Hz, 1H), 8.30 (d, J=9.8 Hz, 1H), 8.04 (d, J=8.3 Hz, 1H), 7.55 (d, J=1.9 Hz, 1H), 7.52-7.43 (m, 2H), 7.20 (d, J=9.8 Hz, 1H), 4.48 (t, J=5.6 Hz, 1H), 4.41-4.32 (m, 1H), 4.08 (s, 3H), 3.71 (s, 2H), 3.57 (s, 1H), 3.51 (s, 2H), 3.26-3.14 (m, 1H), 2.88 (t, J=5.5 Hz, 1H), 2.82 (t, J=5.7 Hz, 1H), 2.13 (dt, J=12.6, 6.4 Hz, 1H), 2.05-1.98 (m, 2H), 1.90-1.81 (m, 3H), 1.48 (m, 2H).¹⁹F NMR: (376 MHz, DMSO-d₆) δ −53.32, −57.20, −217.33.

### Example 89: Synthesis of Compound 314

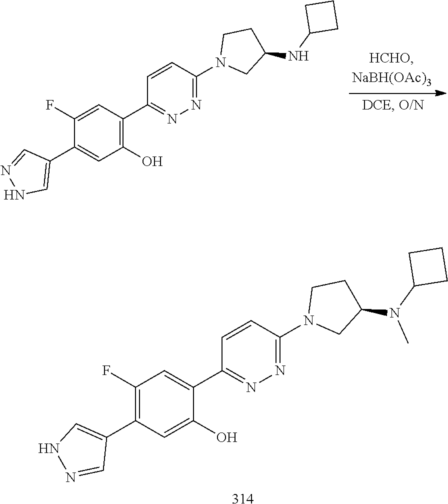 [SMILES: Oc1cc(-c2cn[nH]c2)c(F)cc1-c1ccc(N2CC[C@@H](NC3CCC3)C2)nn1 ; CN(C1CCC1)[C@@H]1CCN(c2ccc(-c3cc(F)c(-c4cn[nH]c4)cc3O)nn2)C1 ; "314"]

To a solution of 2-{6-[(3R)-3-(cyclobutylamino)pyrrolidin-1-yl]pyridazin-3-yl}-4-fluoro-5-(1H-pyrazol-4-yl)phenol (80 mg, 0.203 mmol, 1 equiv) and HCHO (7.31 mg, 0.244 mmol, 1.2 equiv) in DCM (3 mL) were added STAB (128.95 mg, 0.609 mmol, 3 equiv) at 0° C. After stirring at r.t overnight. under a nitrogen atmosphere, the resulting mixture was quenched with ice water(1 mL). The resulting mixture was concentrated under reduced pressure. The crude product was purified by Chiral-Prep-HPLC with the following conditions (2 #SHIMADZU (HPLC-01)): Column: Xselect CSH C18 OBD Column 30*150 mm 5 m, n; Mobile Phase A: Water (0.05% HCl), Mobile Phase B: ACN; Flow rate: 60 mL/min; Gradient: 5% B to 30% B in 8 min, 30% B; Wave Length: UV 220 nm; RT1(min): 6.24 to afford 2-{6-[(3R)-3-[cyclobutyl(methyl)amino]pyrrolidin-1-yl]pyridazin-3-yl}-4-fluoro-5-(1H-pyrazol-4-yl)phenol (21.9 mg, 26%) as a solid. LCMS (ES, m/z): 408.9 [M+H]^{+ 1}H NMR (400 MHz, DMSO-d₆) δ 13.62 (s, 1H), 13.12 (s, 1H), 8.23 (d, J=9.9 Hz, 2H), 7.99 (s, 1H), 7.81 (d, J=12.6 Hz, 1H), 7.29 (d, J=7.0 Hz, 1H), 7.17 (d, J=9.7 Hz, 1H), 3.71 (d, J=9.7 Hz, 2H), 3.47-3.34 (m, 1H), 3.27 (dd, J=10.5, 8.3 Hz, 1H), 3.20-3.08 (m, 1H), 3.07-2.94 (m, 1H), 2.09 (s, 4H), 1.99 (dd, J=7.0, 3.3 Hz, 2H), 1.92-1.80 (m, 3H), 1.58 (dtd, J=10.5, 6.2, 3.2 Hz, 2H).

### Example 90: Synthesis of Compound 321

### Synthesis of Intermediate B223

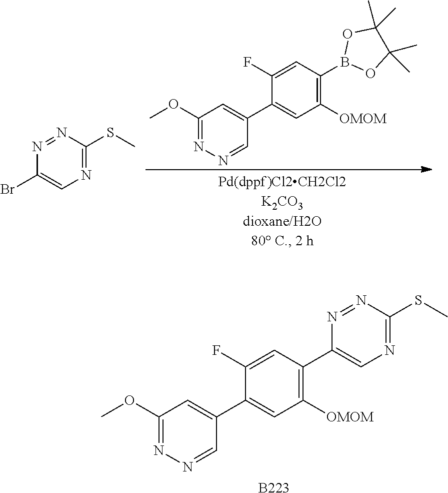

A solution of 6-bromo-3-(methylsulfanyl)-1,2,4-triazine (500 mg, 2.426 mmol, 1 equiv), 5-[2-fluoro-5-(methoxymethoxy)-4-(4,4,5,5-tetramethyl-1,3,2-dioxaborolan-2-yl)phenyl]-3-methoxypyridazine (1893.72 mg, 4.852 mmol, 2 equiv), Pd(dppf)Cl₂·CH₂Cl₂ (197.67 mg, 0.243 mmol, 0.1 equiv) and K₂CO₃ (1006.05 mg, 7.278 mmol, 3 equiv) in 1,4-dioxane and H₂O (4:1,3 mL) was stirred for 2 h at 80° C. under nitrogen atmosphere. The resulting mixture was extracted with EtOAc (5×20 mL). The combined organic layers were dried over anhydrous Na₂SO₄. After filtration, the filtrate was concentrated under reduced pressure. The residue was purified by silica gel column chromatography, eluted with PE/EA (1:1) to afford 6-[5-fluoro-2-(methoxymethoxy)-4-(6-methoxypyridazin-4-yl)phenyl]-3-(methylsulfanyl)-1,2,4-triazine (300 mg, 32%) as a solid.

### Synthesis of Intermediate B224

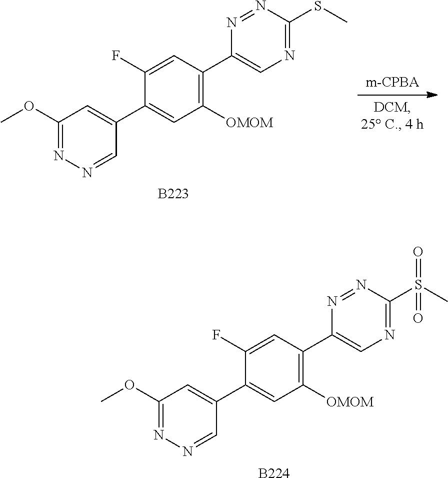

To a stirred solution of 6-[5-fluoro-2-(methoxymethoxy)-4-(6-methoxypyridazin-4-yl)phenyl]-3-(methylsulfanyl)-1,2,4-triazine (200 mg, 0.514 mmol, 1 equiv) in DCM(2 mL) was added m-CPBA (132.94 mg, 0.771 mmol, 1.5 equiv) in portions at 0° C. The resulting mixture was stirred for 4 h at room temperature. The resulting mixture was concentrated under reduced pressure. The crude product (200 mg, 60% purity) resulting mixture was used in the next step directly without further purification.

### Synthesis of Intermediate B225

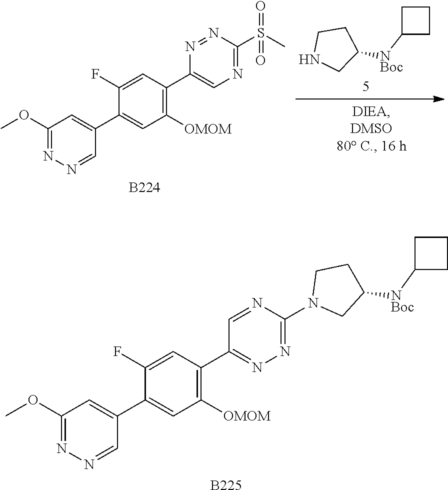

Into an 8 mL vial were added intermediate B224 crude mixture (200 mg, 0.475 mmol, 1.00 equiv), tert-butyl (S)-cyclobutyl(pyrrolidin-3-yl)carbamate (114 mg, 0.475 mmol, 1 equiv), DMSO (2 mL), DIEA (183.825 mg, 1.425 mmol, 3 equiv) at room temperature. The resulting mixture was stirred for 16 h at 80° C. The reaction was quenched by the addition of H₂O (5 mL) at room temperature. The resulting mixture was extracted with EA (3×5 mL). The combined organic layers were washed with saturated salt water (1×30 mL), dried over anhydrous Na₂SO₄.

After filtration, the filtrate was concentrated under reduced pressure. The residue was purified by Prep-TLC (DCMIMeOH=10:1) to afford tert-butyl (S)-cyclobutyl(1-(6-(5-fluoro-2-(methoxymethoxy)-4-(6-methoxypyridazin-4-yl)phenyl)-1,2,4-triazin-3-yl)pyrrolidin-3-yl)carbamate (80 mg) as a solid.

### Synthesis of Compound 321

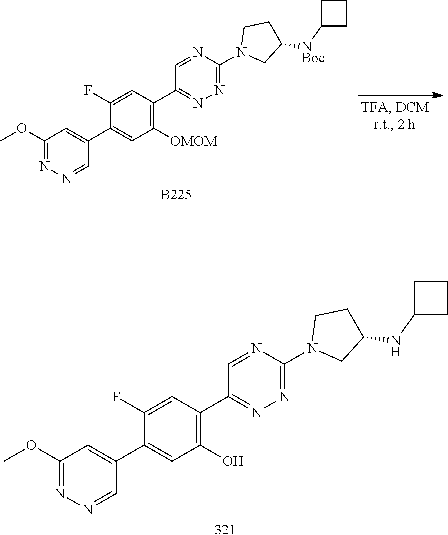

To a solution of tert-butyl N-cyclobutyl-N-[(3S)-1-{6-[5-fluoro-2-(methoxymethoxy)-4-(6-methoxypyridazin-4-yl)phenyl]-1,2,4-triazin-3-yl}pyrrolidin-3-yl]carbamate (80 mg, 0.138 mmol, 1 equiv) in DCM (3 mL) were added TFA (1 mL). After stirring for 2 h at 25° C. under a nitrogen atmosphere, the resulting mixture was concentrated under reduced pressure. The crude product was purified by Chiral-Prep-HPLC with the following conditions (2 #SHIMADZU (HPLC-01)): Column, CHIRALPAK IG, 2*25 cm, 5 um; mobile phase, MtBE (0.1% DEA) and MeOH- (hold 50% MeOH- in 18 min) to afford 2-{3-[(3S)-3-(cyclobutylamino)pyrrolidin-1-yl]-1,2,4-triazin-6-yl}-4-fluoro-5-(6-methoxypyridazin-4-yl)phenol (10.5 mg, 17%) as a solid. LCMS (ESI, m/z): 438 [M+H]^{+ 1}H NMR (400 MHz, DMSO-d₆) δ 11.32 (s, 1H), 9.13 (t, J=1.8 Hz, 1H), 9.08 (s, 1H), 7.89 (d, J=11.9 Hz, 1H), 7.41 (dd, J=1.8, 1.0 Hz, 1H), 7.24 (d, J=6.7 Hz, 1H), 4.10 (s, 3H), 3.65 (t, J=40.7 Hz, 3H), 3.39 (s, 1H), 3.29-3.19 (m, 2H), 2.14 (td, J=7.4, 2.8 Hz, 3H), 1.88 (d, J=26.0 Hz, 1H), 1.75-1.65 (m, 2H), 1.62-1.46 (m, 2H).

### Example 91: Synthesis of compound 325

### Synthesis of Intermediate B226

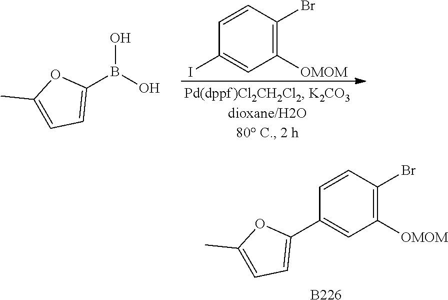

To a solution of 5-methylfuran-2-ylboronic acid (1 g, 7.942 mmol, 1 equiv) and 1-bromo-4-iodo-2-(methoxymethoxy)benzene (2.72 g, 7.942 mmol, 1 equiv) in 1,4-dioxane (2 mL) and H₂O (0.5 mL) were added K₂CO₃ (2.74 g, 19.855 mmol, 2.5 equiv) and Pd(dppf)Cl₂CH₂Cl₂ (0.32 g, 0.397 mmol, 0.05 equiv). After stirring for 2 h at 80° C. under a nitrogen atmosphere, the resulting mixture was concentrated under reduced pressure. The residue was purified by Prep-TLC/silica gel column chromatography, eluted with PE/EA (1:1) to afford 2-[4-bromo-3-(methoxymethoxy)phenyl]-5-methylfuran (600 mg, 25%) as a solid.

### Synthesis of Intermediate B227

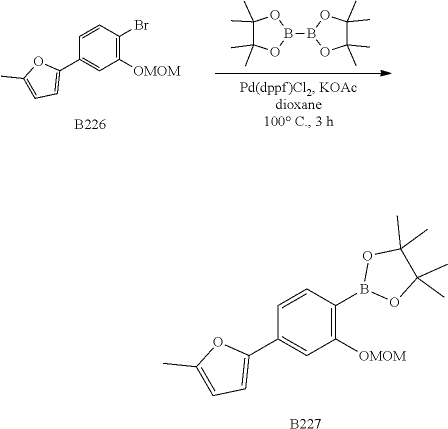

To a solution of 2-[4-bromo-3-(methoxymethoxy)phenyl]-5-methylfuran (600 mg, 2.019 mmol, 1 equiv) and bis(pinacolato)diboron (769.13 mg, 3.029 mmol, 1.5 equiv) in 1,4-dioxane (3 mL) were added KOAc (594.5 mg, 6.057 mmol, 3 equiv) and Pd(dppf)Cl₂ (73.87 mg, 0.101 mmol, 0.05 equiv). After stirring for 3 h at 100° C. under a nitrogen atmosphere. The resulting mixture was filtered and the filter cake was washed with DCM (3×10 mL). The filtrate was concentrated under reduced pressure. to afford 2-[2-(methoxymethoxy)-4-(5-methylfuran-2-yl)phenyl]-4,4,5,5-tetramethyl-1,3,2-dioxaborolane (600 mg, 86%) as an oil.

### Synthesis of Intermediate B228

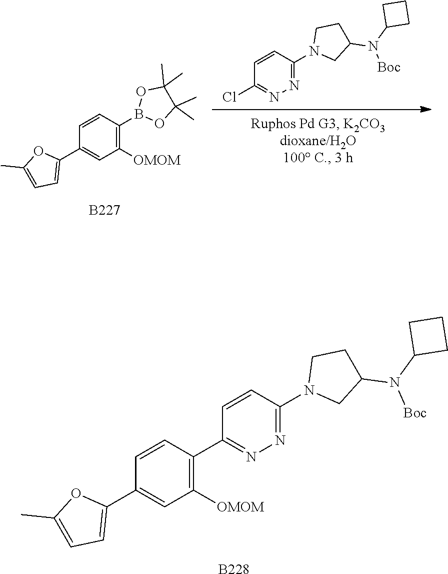

To a solution of 2-[2-(methoxymethoxy)-4-(5-methylfuran-2-yl)phenyl]-4,4,5,5-tetramethyl-1,3,2-dioxaborolane (300 mg, 0.872 mmol, 1 equiv) and tert-butyl N-[1-(6-chloropyridazin-3-yl)pyrrolidin-3-yl]-N-cyclobutylcarbamate (338.29 mg, 0.959 mmol, 1.1 equiv) in 1,4-dioxane (2 mL) and H₂O (0.5 mL, 27.755 mmol, 31.84 equiv) were added K₂CO₃ (361.36 mg, 2.616 mmol, 3 equiv) and RuPhos Palladacycle Gen.3 (72.9 mg, 0.087 mmol, 0.1 equiv). After stirring for 3 h at 100° C. under a nitrogen atmosphere, the resulting mixture was concentrated under reduced pressure. The residue was purified by Prep-TLC/silica gel column chromatography, eluted with CHCl₃/MeOH (10:1) to afford tert-butyl N-cyclobutyl-N-(1-{6-[2-(methoxymethoxy)-4-(5-methylfuran-2-yl)phenyl]pyridazin-3-yl}pyrrolidin-3-yl)carbamate (70 mg, 15%) as a solid.

### Synthesis of Compound 325

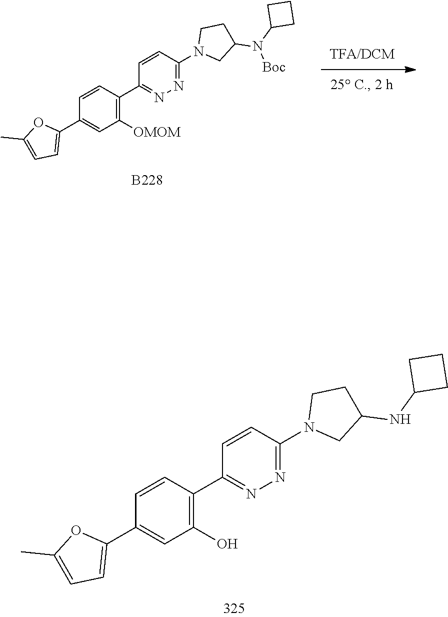

To a solution of tert-butyl N-cyclobutyl-N-(1-{6-[2-(methoxymethoxy)-4-(5-methylfuran-2-yl)phenyl]pyridazin-3-yl}pyrrolidin-3-yl)carbamate (70 mg, 0.131 mmol, 1 equiv) in DCM (1 mL, 15.731 mmol, 120.15 equiv) were added TFA (0.5 mL, 6.732 mmol, 51.42 equiv). After stirring for 2 h at 25° C. under a nitrogen atmosphere, the resulting mixture was concentrated under reduced pressure. The crude product was purified by Chiral-Prep-HPLC with the following conditions (2 #SHIMADZU (HPLC-01)): Column, Kinetex EVO prep C18, 30*150, 5 um; mobile phase, Water (10 mmol/L NH₄HCO₃) and ACN (25% ACN up to 65% in 8 min) to afford the crude product. The crude product was purified by Chiral-Prep-HPLC with the following conditions (2 #SHIMADZU (HPLC-01)): Column, Kinetex EVO prep C18, 30*150, 5 um; mobile phase, Water (10 mmol/L NH₄HCO₃) and ACN (0.1% DEA)-merk (25% ACN (0.1% DEA)-merk up to 67% in 8 min) to afford 2-{6-[3-(cyclobutylamino)pyrrolidin-1-yl]pyridazin-3-yl}-5-(5-methylfuran-2-yl)phenol (14.4 mg, 28%) as a solid. LCMS (ES, m/z): 390.9 [M+H]^{+ 1}H NMR (400 MHz, DMSO-d₆) δ 13.94 (s, 1H), 8.20 (d, J=9.8 Hz, 1H), 7.88 (d, J=8.3 Hz, 1H), 7.30-7.16 (m, 2H), 7.13 (d, J=9.7 Hz, 1H), 6.89 (d, J=3.2 Hz, 1H), 6.22 (d, J=3.2 Hz, 1H), 3.62 (dt, J=16.0, 7.8 Hz, 2H), 3.54-3.43 (m, 1H), 3.38 (q, J=5.6 Hz, 1H), 3.24 (p, J=7.5 Hz, 2H), 2.36 (s, 3H), 2.23-2.08 (m, 3H), 2.07-2.02 (m, 1H), 1.82 (dt, J 12.4, 6.5 Hz, 1H), 1.73-1.63 (m, 2H), 1.59 (ddd, J=12.5, 6.3, 2.5 Hz, 2H).

### Example 92: Synthesis of Compound 322 and 330

### Synthesis of Intermediate B229

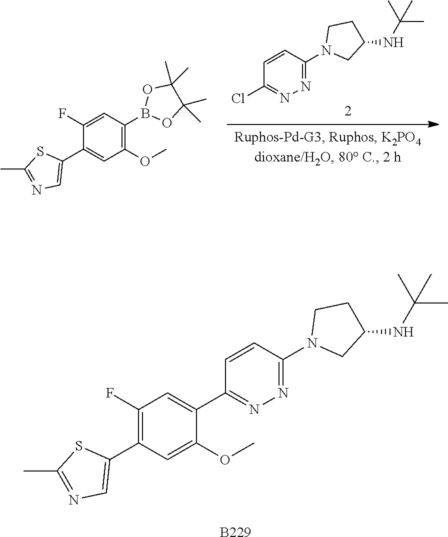

To a solution of 5-[2-fluoro-5-methoxy-4-(4,4,5,5-tetramethyl-1,3,2-dioxaborolan-2-yl) phenyl]-2-methyl-1,3-thiazole (200 mg, 0.573 mmol, 1 equiv) and (3S)—N-tert-butyl-1-(6-chloropyridazin-3-yl) pyrrolidin-3-amine (145.9 mg, 0.573 mmol, 1.0 equiv) in 1,4-dioxane (2 mL) and H₂O (400 uL) were added K₃PO₄ (303.9 mg, 1.432 mmol, 2.5 equiv) and RuPhos Palladacycle Gen.3 (47.9 mg, 0.057 mmol, 0.1 equiv) and RuPhos (26.72 mg, 0.057 mmol, 0.1 equiv). After stirring for 2 h at 80° C. under a nitrogen atmosphere, the resulting mixture was concentrated under reduced pressure. The resulting mixture was extracted with EtOAc (3×50 mL). The combined organic layers were washed with brine (3×30 mL), dried over anhydrous Na₂SO₄. After filtration, the filtrate was concentrated under reduced pressure. The residue was purified by Prep-TLC (PE/EA 3:1) to afford (3S)—N-tert-butyl-1-{6-[5-fluoro-2-methoxy-4-(2-methyl-1,3-thiazol-5-yl) phenyl]pyridazin-3-yl}pyrrolidin-3-amine (150 mg, 59%) as a solid.

### Synthesis of Compound 330

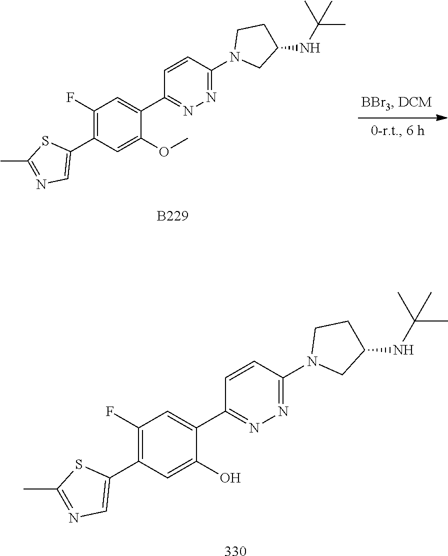

A solution of (3S)—N-tert-butyl-1-{6-[5-fluoro-2-methoxy-4-(2-methyl-1,3-thiazol-5-yl) phenyl]pyridazin-3-yl}pyrrolidin-3-amine (130 mg, 0.294 mmol, 1 equiv) in DCM (2 mL) was treated with BBr₃ (147.51 mg, 0.588 mmol, 2.0 equiv) at 0° C. under nitrogen atmosphere. A mixture was stirred for 6 h at room temperature. The crude product was purified by Chiral-Prep-HPLC with the following conditions: Column, Kinetex EVO prep C18, 30×150, 5 um; mobile phase, Water (10 mmol/L NH₄HCO₃) and ACN (20% ACN up to 60% in 8 min); Detector, UV 254 nm. This resulted in 2-{6-[(3S)-3-(tert-butylamino) pyrrolidin-1-yl]pyridazin-3-yl}-4-fluoro-5-(2-methyl-1,3-thiazol-5-yl) phenol (2.1 mg, 2%) as a solid.

### Synthesis of Compound 322

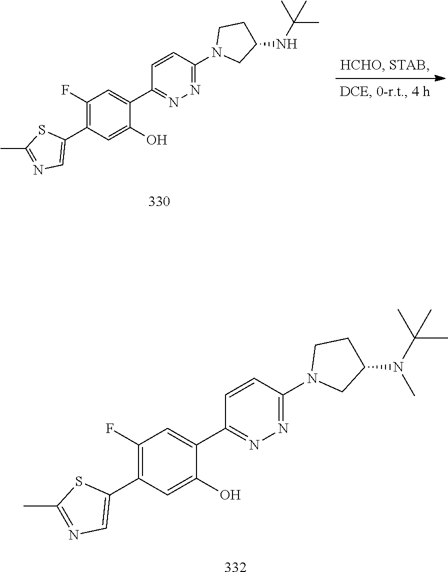

A solution of 2-{6-[(3S)-3-(tert-butylamino) pyrrolidin-1-yl]pyridazin-3-yl}-4-fluoro-5-(2-methyl-1,3-thiazol-5-yl) phenol (70 mg, 0.164 mmol, 1 equiv) in DCE (7 mL) was treated with HCHO (7.37 mg, 0.246 mmol, 1.5 equiv) for 30 min at room temperature under nitrogen atmosphere followed by the addition of STAB (104.1 mg, 0.492 mmol, 3.0 equiv) in portions at 0° C. The resulting mixture was stirred for 4 h at room temperature under nitrogen atmosphere. The crude product was purified by Chiral-Prep-HPLC with the following conditions: Column, Kinetex EVO C18 Column, 30×150.5 um; mobile phase, Water (10 mmol/L NH₄HCO₃) and ACN (20% ACN up to 75% in 8 min); Detector, UV 254 nm. This resulted in 2-{6-[(3S)-3-[tert-butyl(methyl)amino]pyrrolidin-1-yl]pyridazin-3-yl}-4-fluoro-5-(2-methyl-1,3-thiazol-5-yl) phenol (13.3 mg, 18.76%) as an off-white solid. Compound 330: LCMS: (ES, m/z): 427 [M+H]+H NMR: (400 MHz, DMSO-d₆) δ13.75 (s, 1H), 8.25 (d, J=9.9 Hz, 1H), 8.16 (s, 1H), 7.93 (d, J=12.7 Hz, 1H), 7.29 (d, J=6.8 Hz, 1H), 7.16 (d, J=9.7 Hz, 1H), 3.78 (d, J=9.4 Hz, 1H), 3.65 (s, 1H), 3.58-3.49 (m, 1H), 3.44 (q, J=9.7, 9.0 Hz, 1H), 3.08 (dd, J=10.4, 7.1 Hz, 1H), 2.71 (s, 3H), 2.18 (ddt, J=10.4, 6.7, 3.6 Hz, 1H), 1.75 (dq, J=11.9, 8.6 Hz, 2H), 1.08 (s, 9H).Compound 322: LCMS: (ES, m/z): 441 [M+H]^{+ 1}H NMR: (400 MHz, DMSO-d₆) δ13.74 (s, 1H), 8.25 (d, J=9.9 Hz, 1H), 8.16 (s, 1H), 7.93 (d, J=12.7 Hz, 1H), 7.29 (d, J=6.8 Hz, 1H), 7.19 (d, J=9.7 Hz, 1H), 4.02-3.89 (m, 1H), 3.71 (t, J=9.7 Hz, 1H), 3.49 (t, J=9.4 Hz, 1H), 3.43-3.36 (m, 1H), 3.30-3.26 (m, 1H), 2.70 (s, 3H), 2.21 (s, 3H), 2.11-1.97 (m, 1H), 1.92 (ddd, J=10.5, 7.7, 5.7 Hz, 1H), 1.10 (s, 9H).

### Example 93: Synthesis of compounds 331 and 332

### Synthesis of Intermediate B230

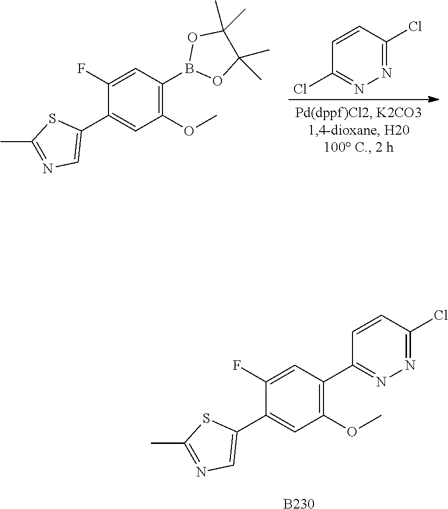

To a solution of 5-[2-fluoro-5-methoxy-4-(4,4,5,5-tetramethyl-1,3,2-dioxaborolan-2-yl)phenyl]-2-methyl-1,3-thiazole (800 mg, 2.291 mmol, 1 equiv) and pyridazine, 3,6-dichloro-(341.25 mg, 2.291 mmol, 1 equiv) in 1,4-dioxane (10 mL) and H₂O (2 mL, 111.019 mmol, 48.46 equiv) were added K₂CO₃ (949.78 mg, 6.873 mmol, 3 equiv) and Pd(dppf)Cl₂ (83.81 mg, 0.115 mmol, 0.05 equiv). After stirring for 2 h at 100° C. under a nitrogen atmosphere, the resulting mixture was concentrated under reduced pressure. The residue was purified by silica gel column chromatography, eluted with PE/EA(1:1) to afford 3-chloro-6-[5-fluoro-2-methoxy-4-(2-methyl-1,3-thiazol-5-yl)phenyl]pyridazine (800 mg, 104%) as a solid.

### Synthesis of Intermediate B231

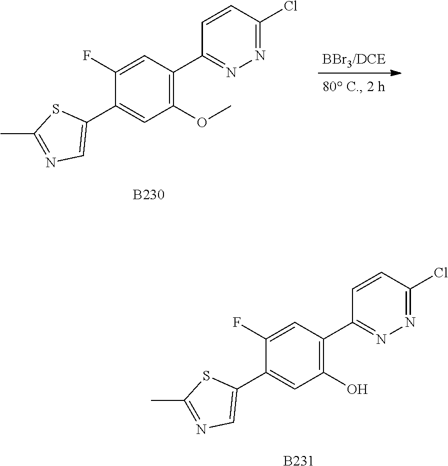 [SMILES: "B231" ; "B230" ; COc1cc(-c2cnc(C)s2)c(F)cc1-c1ccc(Cl)nn1 ; Cc1ncc(-c2cc(O)c(-c3ccc(Cl)nn3)cc2F)s1]

To a solution of 3-chloro-6-[5-fluoro-2-methoxy-4-(2-methyl-1,3-thiazol-5-yl)phenyl]pyridazine (800 mg, 2.383 mmol, 1 equiv) in DCE (20 mL) was added BBr₃ (2 mL, 21.156 mmol, 8.88 equiv) at 0° C. After stirring for 2 h at 80° C. under a nitrogen atmosphere. The mixture was allowed to cool down to 0° C. The reaction was quenched by the addition of MeOH (10 mL) at 0° C., the resulting mixture was concentrated under reduced pressure. The residue was dissolved in H₂O (20 mL), the resulting mixture was extracted with EA (3×10 mL). The combined organic layers were washed with brine (1×10 mL), dried over anhydrous Na₂SO₄. After filtration, the filtrate was concentrated under reduced pressure to afford crude 2-(6-chloropyridazin-3-yl)-4-fluoro-5-(2-methyl-1,3-thiazol-5-yl)phenol (600 mg, 78%) as a solid.

### Synthesis of Intermediate B232

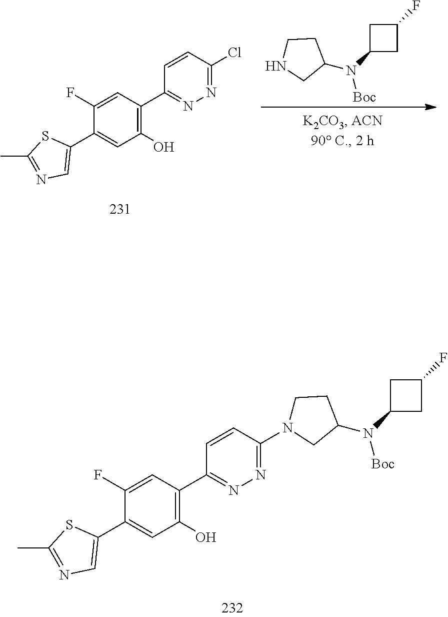 [SMILES: Cc1ncc(-c2cc(O)c(-c3ccc(Cl)nn3)cc2F)s1 ; CC(C)(C)OC(=O)N(C1CCNC1)[C@H]1C[C@H](F)C1 ; "231" ; Cc1ncc(-c2cc(O)c(-c3ccc(N4CCC(N(C(=O)OC(C)(C)C)[C@H]5C[C@H](F)C5)C4)nn3)cc2F)s1]

To a solution of 2-(6-chloropyridazin-3-yl)-4-fluoro-5-(2-methyl-1,3-thiazol-5-yl)phenol (300 mg, 0.932 mmol, 1 equiv) and tert-butyl N-(pyrrolidin-3-yl)-N-[(1r,3r)-3-fluorocyclobutyl]carbamate (240.87 mg, 0.932 mmol, 1 equiv) in ACN (3 mL, 57.073 mmol, 61.21 equiv) were added K₂CO₃ (386.59 mg, 2.796 mmol, 3 equiv). After stirring for 2 h at 90° C. under a nitrogen atmosphere. The mixture was allowed to cool down to 25° C. The resulting mixture was filtered and the filter cake was washed with DCM (3×5 mL). The filtrate was concentrated under reduced pressure. The residue was purified by silica gel column chromatography, eluted with CH₂Cl₂/MeOH (10:1) to afford tert-butyl N-(1-{6-[5-fluoro-2-hydroxy-4-(2-methyl-1,3-thiazol-5-yl)phenyl]pyridazin-3-yl}pyrrolidin-3-yl)-N-[(1r,3r)-3-fluorocyclobutyl]carbamate (30 mg, 6%) as a solid.

### Synthesis of Intermediate B233

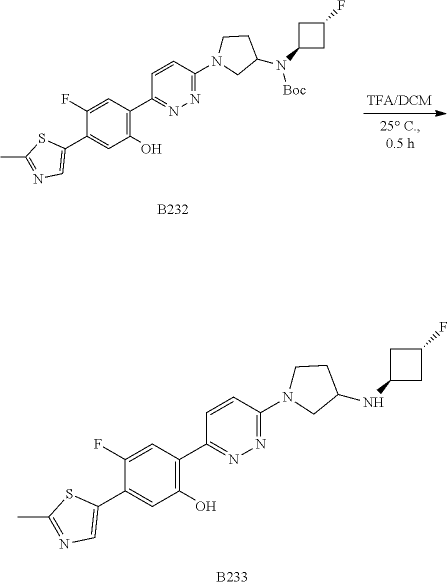 [SMILES: "B232" ; "B233" ; Cc1ncc(-c2cc(O)c(-c3ccc(N4CCC(N(C(=O)OC(C)(C)C)[C@H]5C[C@H](F)C5)C4)nn3)cc2F)s1 ; Cc1ncc(-c2cc(O)c(-c3ccc(N4CCC(N[C@H]5C[C@H](F)C5)C4)nn3)cc2F)s1]

To a solution of tert-butyl N-(1-{6-[5-fluoro-2-hydroxy-4-(2-methyl-1,3-thiazol-5-yl)phenyl]pyridazin-3-yl}pyrrolidin-3-yl)-N-[(1r,3r)-3-fluorocyclobutyl]carbamate (30 mg, 0.055 mmol, 1 equiv) in DCM (1 mL, 15.731 mmol, 285.05 equiv) was added TFA (0.25 mL, 3.366 mmol, 60.99 equiv). After stirring for 0.5 h at 25° C. under a nitrogen atmosphere, the resulting mixture was concentrated under reduced pressure. The crude product was purified by Chiral-Prep-HPLC with the following conditions (2 #SHIMADZU (HPLC-01)): Column, XBridge Shield RP18 OBD Column, 30*150 mm, 5 μm; mobile phase, Water (10 mmol/L NH₄HCO₃) and ACN (20% ACN up to 60% in 10 min) to afford 4-fluoro-5-(2-methyl-1,3-thiazol-5-yl)-2-[6-(3- {[(1r,3r)-3-fluorocyclobutyl]amino}pyrrolidin-1-yl)pyridazin-3-yl]phenol as a solid.

### Synthesis of Compound 331 and 332

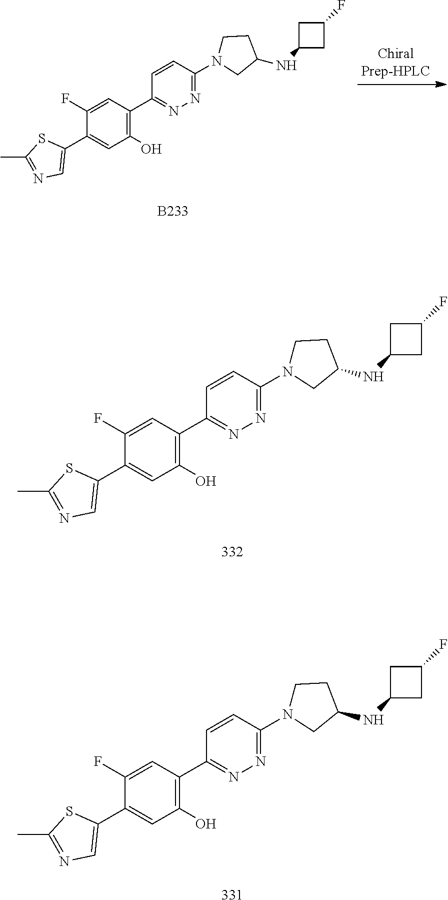 [SMILES: "B233" ; Cc1ncc(-c2cc(O)c(-c3ccc(N4CCC(N[C@H]5C[C@H](F)C5)C4)nn3)cc2F)s1 ; Cc1ncc(-c2cc(O)c(-c3ccc(N4CC[C@H](N[C@H]5C[C@H](F)C5)C4)nn3)cc2F)s1 ; "332" ; Cc1ncc(-c2cc(O)c(-c3ccc(N4CC[C@@H](N[C@H]5C[C@H](F)C5)C4)nn3)cc2F)s1 ; "331"]

The 4-fluoro-5-(2-methyl-1,3-thiazol-5-yl)-2-[6-(3-{[(1r,3r)-3-fluorocyclobutyl]amino}pyrrolidin-1-yl)pyridazin-3-yl]phenol (10 mg, 0.023 mmol, 1 equiv) was purified by Chiral-Prep-HPLC with the following conditions (2 #SHIMADZU (HPLC-01)): Column, CHIRALPAK IA, 2*25 cm, 5 um; mobile phase, MtBE(0.1% DEA) and MeOH- (hold 50% MeOH- in 13.5 min) to afford 4-fluoro-2-(6-((S)-3-(((1r,3R)-3-fluorocyclobutyl)amino)pyrrolidin-1-yl)pyridazin-3-yl)-5-(2-methylthiazol-5-yl)phenol (1.5 mg, 15.00%) as a white solid, 4-fluoro-2-(6-((R)-3-(((1r,3R)-3-fluorocyclobutyl)amino)pyrrolidin-1-yl)pyridazin-3-yl)-5-(2-methylthiazol-5-yl)phenol (1.7 mg, 17.00%) as a solid. Compound 332: LCMS:(ES, m/z): 443.8 [M+H]^{+ 1}H NMR: (400 MHz, DMSO-d₆) δ 13.72 (s, 1H), 8.25 (d, J=9.9 Hz, 1H), 8.15 (s, 1H), 7.93 (d, J=12.7 Hz, 1H), 7.29 (d, J=6.8 Hz, 1H), 7.17 (d, J=9.8 Hz, 1H), 5.18 (ddt, J=56.9, 6.3, 2.9 Hz, 1H), 3.73-3.59 (m, 2H), 3.57-3.45 (m, 2H), 3.37 (t, J=5.7 Hz, 1H), 3.29 (s, 1H), 2.71 (s, 3H), 2.43-2.24 (m, 2H), 2.15 (ddd, J=26.2, 10.8, 5.5 Hz, 3H), 1.88-1.77 (m, 1H). Compound 331: LCMS:(ES, m/z): 443.8 [M+H]^{+ 1}HNMR: (400 MHz, DMSO-d₆) δ 13.72 (s, 1H), 8.25 (d, J=9.9 Hz, 1H), 8.15 (s, 1H), 7.93 (d, J=12.7 Hz, 1H), 7.29 (d, J=6.8 Hz, 1H), 7.17 (d, J=9.8 Hz, 1H), 5.18 (ddt, J=56.9, 6.3, 2.9 Hz, 1H), 3.73-3.59 (m, 2H), 3.57-3.45 (m, 2H), 3.37 (t, J=5.7 Hz, 1H), 3.29 (s, 1H), 2.71 (s, 3H), 2.43-2.24 (m, 2H), 2.15 (ddd, J=26.2, 10.8, 5.5 Hz, 3H), 1.88-1.77 (m, 1H).

### Example 94: Synthesis of Compound 334

### Synthesis of Intermediate B234

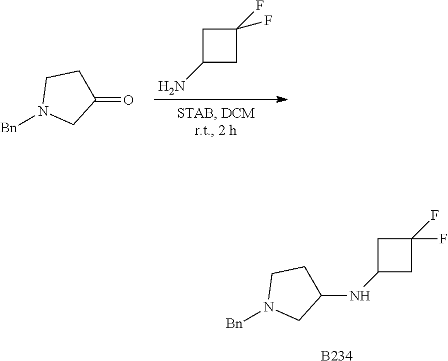 [SMILES: "B234" ; O=C1CCN(Cc2ccccc2)C1 ; NC1CC(F)(F)C1 ; FC1(F)CC(NC2CCN(Cc3ccccc3)C2)C1]

To a solution of 1-benzylpyrrolidin-3-one (1 g, 5.707 mmol, 1 equiv) in DCM (10 mL) was added 3,3-difluorocyclobutan-1-amine (0.61 g, 5.707 mmol, 1 equiv) and STAB (2.42 g, 11.414 mmol, 2 equiv) at 0° C. After stirring for 3 h at 25° C. under a nitrogen atmosphere. The reaction was quenched by the addition of ice water(10 mL) at 0° C. The mixture was basified to pH 10 with Na₂CO₃. The resulting mixture was extracted with CH₂Cl₂ (3×10 mL). The combined organic layers were washed with brine (1×10 mL), dried over anhydrous Na₂SO₄. After filtration, the filtrate was concentrated under reduced pressure to afford crude 1-benzyl-N-(3,3-difluorocyclobutyl)pyrrolidin-3-amine (1 g, 66%) as an oil.

### Synthesis of Intermediate B235

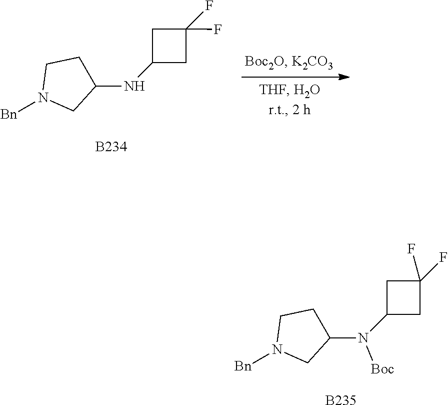 [SMILES: "B234" ; "B235" ; FC1(F)CC(NC2CCN(Cc3ccccc3)C2)C1 ; CC(C)(C)OC(=O)N(C1CCN(Cc2ccccc2)C1)C1CC(F)(F)C1]

To a solution of 1-benzyl-N-(3,3-difluorocyclobutyl)pyrrolidin-3-amine (1 g, 3.75 mmol, 1 equiv) in THF (10 mL) were added K₂CO₃ (1.04 g, 7.51 mmol, 2 equiv) in H₂O (2 mL) and Boc₂O (1.23 g, 5.6 mmol, 1.5 equiv) was added to the reaction. After stirring for 2 h at 25° C. under a nitrogen atmosphere. The resulting mixture was extracted with EtOAc (3×30 mL). The combined organic layers were washed with brine (1×10 mL), dried over anhydrous Na₂SO₄. After filtration, the filtrate was concentrated under reduced pressure. the resulting mixture was concentrated under reduced pressure. The residue was purified by Prep-TLC/silica gel column chromatography, eluted with PE/EA (3:1) to afford tert-butyl (1-benzylpyrrolidin-3-yl)(3,3-difluorocyclobutyl)carbamate (1 g, 73%) as an oil.

### Synthesis of Intermediate B236

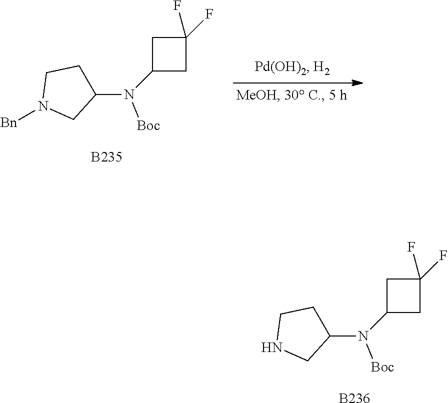

To a solution of tert-butyl (1-benzylpyrrolidin-3-yl)(3,3-difluorocyclobutyl)carbamate (1 g, 2.729 mmol, 1 equiv) in MeOH (10 mL) was added Pd(OH)2/C (0.3 g) under nitrogen atmosphere in a 100 mL round-bottom flask. The mixture was hydrogenated at 30° C. for 5 h under hydrogen atmosphere using a hydrogen balloon. The resulting mixture was filtered and the filter cake was washed with MeOH (30×10 mL). The filtrate was concentrated under reduced pressure to afford tert-butyl (3,3-difluorocyclobutyl)(pyrrolidin-3-yl)carbamate (600 mg, 80%) as an oil.

### Synthesis of Intermediate B237

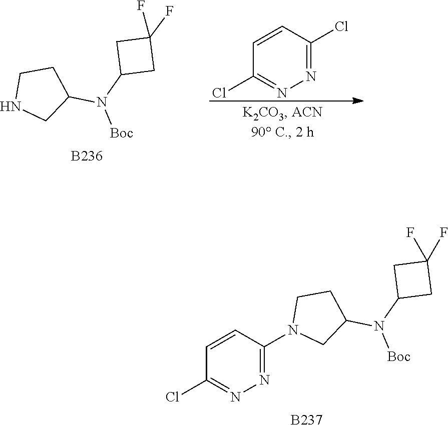

To a solution of tert-butyl (3,3-difluorocyclobutyl)(pyrrolidin-3-yl)carbamate (300 mg, 1.086 mmol, 1 equiv) and pyridazine, 3,6-dichloro- (161.73 mg, 1.086 mmol, 1 equiv) in ACN (5 mL) were added K₂CO₃ (450.13 mg, 3.258 mmol, 3 equiv). After stirring for 2 h at 90° C. under a nitrogen atmosphere. The mixture was allowed to cool down to 25° C. The resulting mixture was filtered and the filter cake was washed with DCM(2×10 mL). The filtrate was concentrated under reduced pressure. The residue was purified by Prep-TLC/silica gel column chromatography, eluted with CH₂Cl₂/ PE (1:1) to afford tert-butyl (1-(6-chloropyridazin-3-yl)pyrrolidin-3-yl)(3,3-difluorocyclobutyl)carbamate (300 mg, 71%) as a solid.

### Synthesis of Intermediate B238

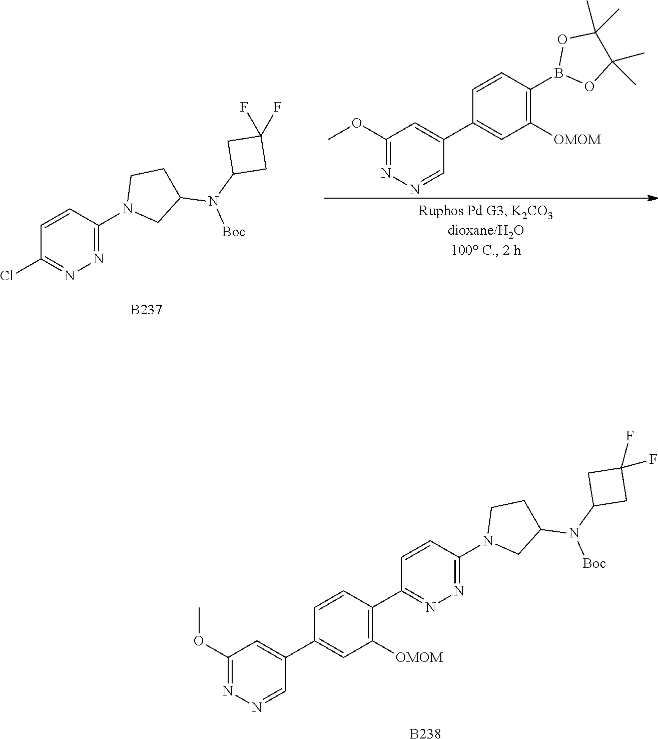

To a solution of tert-butyl (1-(6-chloropyridazin-3-yl)pyrrolidin-3-yl)(3,3-difluorocyclobutyl)carbamate (100 mg, 0.257 mmol, 1 equiv) and 3-methoxy-5-[3-(methoxymethoxy)-4-(4,4,5,5-tetramethyl-1,3,2-dioxaborolan-2-yl)phenyl]pyridazine (105.3 mg, 0.283 mmol, 1.1 equiv) in 1,4-dioxane (2 mL) and H₂O (0.4 mL, 22.204 mmol, 81.01 equiv) were added 3rd Generation RuPhos precatalyst (21.51 mg, 0.026 mmol, 0.1 equiv) and K₂CO₃ (106.63 mg, 0.771 mmol, 3 equiv). After stirring for 2 h at 100° C. under a nitrogen atmosphere, the resulting mixture was concentrated under reduced pressure. The residue was purified by silica gel column chromatography, eluted with PE/EA (1/7) to afford tert-butyl (3,3-difluorocyclobutyl)(1-(6-(2-(methoxymethoxy)-4-(6-methoxypyridazin-4-yl)phenyl)pyridazin-3-yl)pyrrolidin-3-yl)carbamate (100 mg, 64.95%) as a yellow solid.

### Synthesis of Compound 334

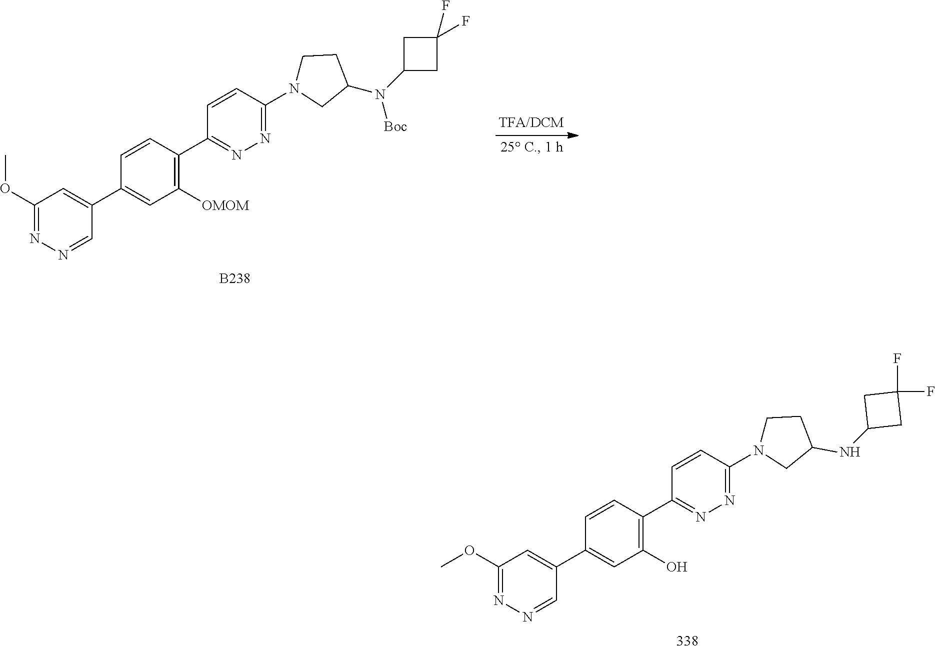

To a solution of tert-butyl N-(3,3-difluorocyclobutyl)-N-(1-{6-[2-(methoxymethoxy)-4-(6-methoxypyridazin-4-yl)phenyl]pyridazin-3-yl}pyrrolidin-3-yl)carbamate (100 mg, 0.167 mmol, 1 equiv) in DCM (3 mL, 47.192 mmol, 282.51 equiv) were added TFA (1 mL, 13.463 mmol, 80.60 equiv). After stirring for 2 h at 25° C. under a nitrogen atmosphere, the resulting mixture was concentrated under reduced pressure. The crude product was purified by Chiral-Prep-HPLC with the following conditions (2 #SHIMADZU (HPLC-01)): Column, Kinetex EVO C18 Column, 30*150.5 um; mobile phase, Water (10 mmol/L NH₄HCO₃) and ACN (20% ACN up to 60% in 8 min) to afford 2-(6-(3-((3,3-difluorocyclobutyl)amino)pyrrolidin-1-yl)pyridazin-3-yl)-5-(6-methoxypyridazin-4-yl)phenol (28.5 mg, 38%) a solid. LCMS:(ES, m/z):454.9[M+H]^{+ 1}H NMR: (400 MHz, DMSO-d₆) δ 14.09 (s, 1H), 9.35 (d, J=1.9 Hz, 1H), 8.31 (d, J=9.9 Hz, 1H), 8.05 (d, J=8.3 Hz, 1H), 7.55 (d, J=1.9 Hz, 1H), 7.50-7.42 (m, 2H), 7.19 (d, J=9.8 Hz, 1H), 4.08 (s, 3H), 3.68 (dd, J=10.7, 5.9 Hz, 1H), 3.63 (d, J=8.0 Hz, 1H), 3.52 (q, J=10.7, 8.8 Hz, 1H), 3.41 (s, 1H), 3.34 (s, 1H), 3.31-3.33 (m, 1H), 2.95-2.67 (m, 2H), 2.47-2.24 (m, 2H), 2.13 (dt, J=12.8, 6.3 Hz, 1H), 1.86 (dd, J=12.5, 6.4 Hz, 1H).

### Example 95: Synthesis of Compound 335

### Synthesis of Intermediate B239

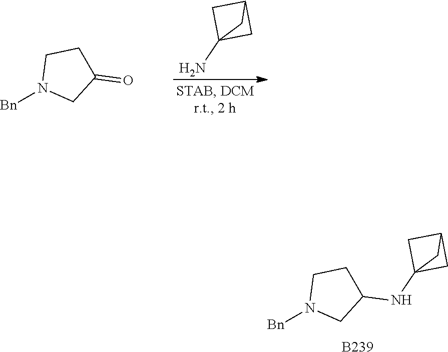

To a solution of 1-benzylpyrrolidin-3-one (1 g, 5.707 mmol, 1 equiv) in DCM (10 mL) was added bicyclo[1.1.1]pentan-1-amine (0.47 g, 5.707 mmol, 1 equiv) and STAB (2.42 g, 11.414 mmol, 2 equiv) at 0° C. After stirring for 3 h at 25° C. under a nitrogen atmosphere. The reaction was quenched by the addition of ice water(10 mL) at 0° C. The mixture was basified to pH 10 with Na₂CO₃. The resulting mixture was extracted with CH₂Cl₂ (3×10 mL). The combined organic layers were washed with brine (1×10 mL), dried over anhydrous Na₂SO₄. After filtration, the filtrate was concentrated under reduced pressure to afford crude 1-benzyl-N-{bicyclo[1.1.1]pentan-1-yl}pyrrolidin-3-amine (1 g, 72%) as an oil.

### Synthesis of Intermediate B240

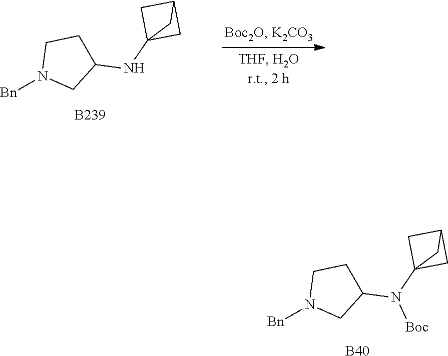 [SMILES: "B239" ; c1ccc(CN2CCC(NC34CC(C3)C4)C2)cc1 ; "B40" ; CC(C)(C)OC(=O)N(C1CCN(Cc2ccccc2)C1)C12CC(C1)C2]

To a solution of 1-benzyl-N-{bicyclo[1.1.1]pentan-1-yl}pyrrolidin-3-amine (1 g, 4.126 mmol, 1 equiv) in THF (10 mL) were added K₂CO₃ (1.14 g, 8.252 mmol, 2 equiv) in H₂O (2 mL) and Boc₂O (1.35 g, 6.189 mmol, 1.5 equiv) was added to the reaction. After stirring for 2 h at 25° C. under a nitrogen atmosphere. The resulting mixture was extracted with EtOAc (3×30 mL). The combined organic layers were washed with brine (1×10 mL), dried over anhydrous Na₂SO₄.

After filtration, the filtrate was concentrated under reduced pressure. the resulting mixture was concentrated under reduced pressure. The residue was purified by Prep-TLC/silica gel column chromatography, eluted with PE/EA (3:1) to afford tert-butyl N-(1-benzylpyrrolidin-3-yl)-N-{bicyclo[1.1.1]pentan-1-yl}carbamate (1 g, 71%) as an oil.

### Synthesis of Intermediate B241

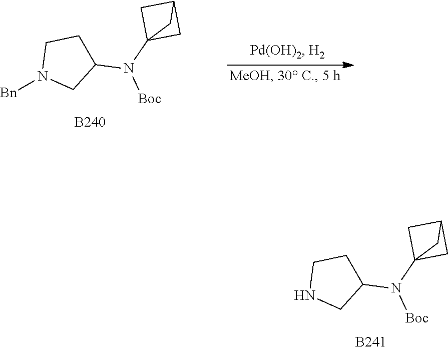

To a solution of tert-butyl N-(1-benzylpyrrolidin-3-yl)-N-{bicyclo[1.1.1]pentan-1-yl}carbamate (1 g, 2.920 mmol, 1 equiv) in MeOH (10 mL) was added Pd(OH)2/C (0.3 g, 2.136 mmol, 0.73 equiv) under nitrogen atmosphere in a 100 mL round-bottom flask. The mixture was hydrogenated at 30° C. for 5 h under hydrogen atmosphere using a hydrogen balloon. The resulting mixture was filtered and the filter cake was washed with MeOH (30×10 mL). The filtrate was concentrated under reduced pressure to afford tert-butyl N-{bicyclo[1.1.1]pentan-1-yl}-N-(pyrrolidin-3-yl)carbamate (600 mg, 81.43%) as an oil.

### Synthesis of Intermediate B242

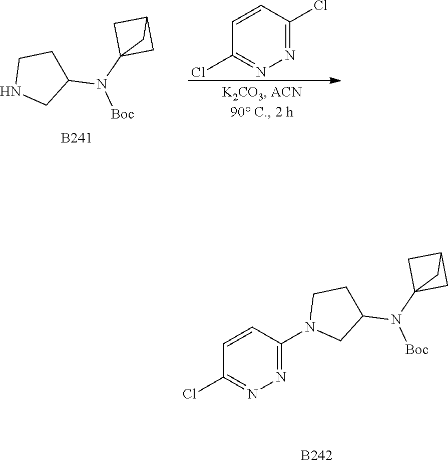 [SMILES: "B241" ; CC(C)(C)OC(=O)N(C1CCNC1)C12CC(C1)C2 ; "B242" ; CC(C)(C)OC(=O)N(C1CCN(c2ccc(Cl)nn2)C1)C12CC(C1)C2 ; Clc1ccc(Cl)nn1]

To a solution of tert-butyl N-{bicyclo[1.1.1]pentan-1-yl}-N-(pyrrolidin-3-yl)carbamate (300 mg, 1.189 mmol, 1 equiv) and pyridazine, 3,6-dichloro- (177.09 mg, 1.189 mmol, 1 equiv) in ACN (5 mL, 95.121 mmol, 80.02 equiv) were added K₂CO₃ (492.89 mg, 3.567 mmol, 3 equiv).

After stirring for 2 h at 90° C. under a nitrogen atmosphere. The mixture was allowed to cool down to 25° C. The resulting mixture was filtered and the filter cake was washed with DCM (2×10 mL). The filtrate was concentrated under reduced pressure. The residue was purified by Prep-TLC/silica gel column chromatography, eluted with CH₂Cl₂/ PE (1:1) to afford tert-butyl N-{bicyclo[1.1.1]pentan-1-yl}-N-[1-(6-chloropyridazin-3-yl)pyrrolidin-3-yl]carbamate (300 mg, 69%) as a solid.

### Synthesis of Intermediate B243

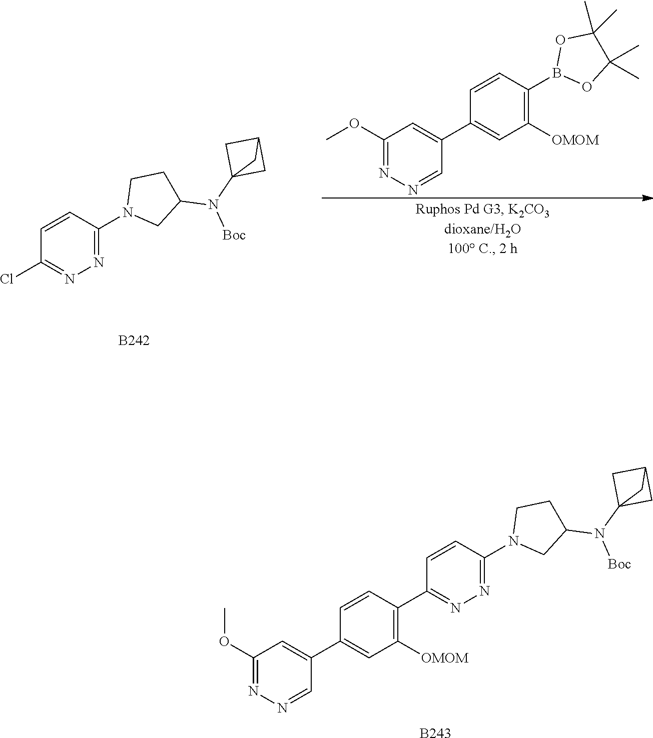

To a solution of tert-butyl N-{bicyclo[1.1.1]pentan-1-yl}-N-[1-(6-chloropyridazin-3-yl)pyrrolidin-3-yl]carbamate (100 mg, 0.274 mmol, 1 equiv) and 3-methoxy-5-[3-(methoxymethoxy)-4-(4,4,5,5-tetramethyl-1,3,2-dioxaborolan-2-yl)phenyl]pyridazine (112.22 mg, 0.301 mmol, 1.1 equiv) in 1,4-dioxane (2 mL) and H₂O (0.4 mL, 22.204 mmol, 81.01 equiv) were added 3rd Generation RuPhos precatalyst (22.92 mg, 0.027 mmol, 0.1 equiv) and K₂CO₃ (113.63 mg, 0.822 mmol, 3 equiv). After stirring for 2 h at 100° C. under a nitrogen atmosphere, the resulting mixture was concentrated under reduced pressure. The residue was purified by Prep-TLC/silica gel column chromatography, eluted with PE/EA (1/7) to afford tert-butyl N-{bicyclo[1.1.1]pentan-1-yl}-N-(1-{6-[2-(methoxymethoxy)-4-(6-methoxypyridazin-4-yl)phenyl]pyridazin-3-yl}pyrrolidin-3-yl)carbamate (100 mg, 63%) as a solid.

### Synthesis of Compound 335

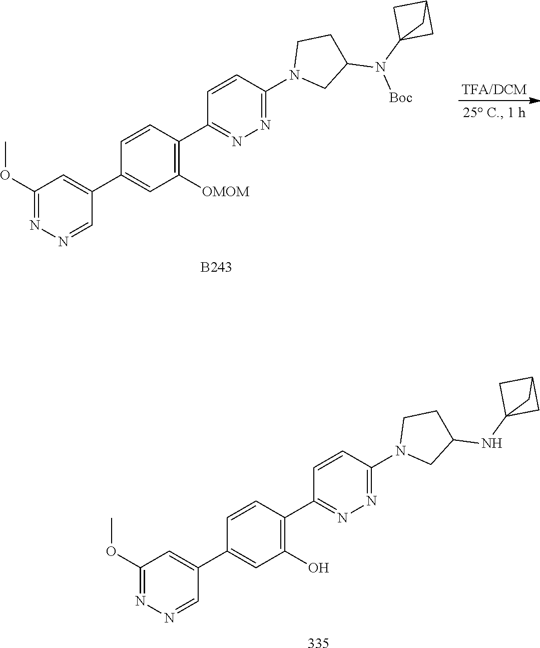

To a solution of tert-butyl N-{bicyclo[1.1.1]pentan-1-yl}-N-(1-{6-[2-(methoxymethoxy)-4-(6-methoxypyridazin-4-yl)phenyl]pyridazin-3-yl}pyrrolidin-3-yl)carbamate (100 mg, 0.174 mmol, 1 equiv) in DCM (3 mL) were added TFA (0.6 mL). After stirring for 2 h at 25° C. under a nitrogen atmosphere, the resulting mixture was concentrated under reduced pressure. The crude product was purified by Chiral-Prep-HPLC with the following conditions (2 #SHIMADZU (HPLC-01)): Column, XBridge Shield RP18 OBD Column, 30*150 mm, 5 μm; mobile phase, Water (10 mmol/L NH₄HCO₃) and ACN (15% ACN up to 65% in 10 min) to afford 2-[6-(3-{bicyclo[1.1.1]pentan-1-ylamino}pyrrolidin-1-yl)pyridazin-3-yl]-5-(6-methoxypyridazin-4-yl)phenol (18.7 mg, 25%) as a solid.LCMS:(ES, m/z):430.9 [M+H]^{+ 1}HNMR: (400 MHz, DMSO-d₆) δ 14.10 (s, 1H), 9.35 (d, J=1.9 Hz, 1H), 8.30 (d, J=9.8 Hz, 1H), 8.04 (d, J=8.3 Hz, 1H), 7.55 (d, J=1.9 Hz, 1H), 7.50 (d, J=1.9 Hz, 1H), 7.46 (d, J=1.9 Hz, 1H), 7.18 (d, J=9.8 Hz, 1H), 4.09 (s, 3H), 3.73 (dd, J=10.8, 6.2 Hz, 1H), 3.62 (d, J=6.3 Hz, 1H), 3.50 (d, J=10.3 Hz, 2H), 3.23 (dd, J=10.9, 5.2 Hz, 1H), 2.78 (s, 1H), 2.36 (s, 1H), 2.23-2.11 (m, 1H), 1.89-1.73 (m, 7H).

### Example 96: Synthesis of Compounds 336 and 337

### Synthesis of Intermediate B244

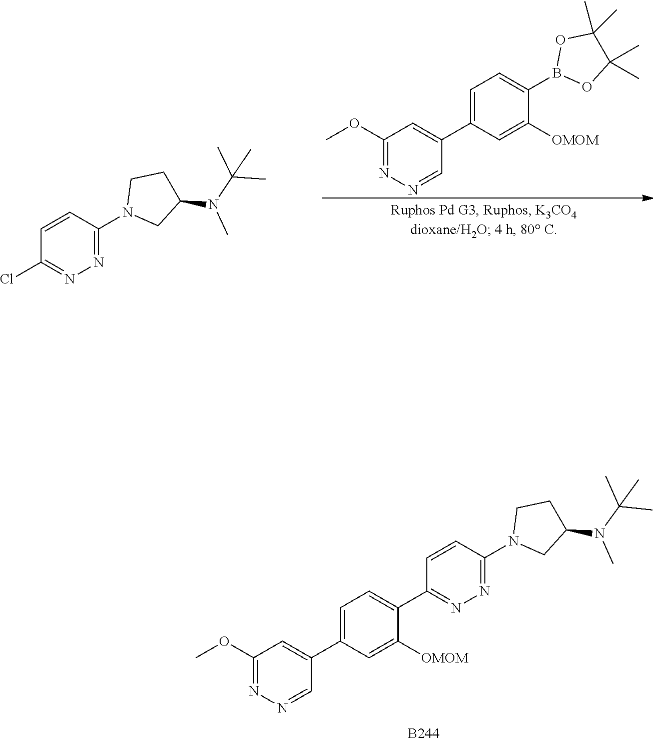

A solution of 3-methoxy-5-[3-(methoxymethoxy)-4-(4,4,5,5-tetramethyl-1,3,2-dioxaborolan-2-yl)phenyl]pyridazine (150 mg, 0.403 mmol, 1 equiv) and (3R)—N-tert-butyl-1-(6-chloropyridazin-3-yl)-N-methylpyrrolidin-3-amine (97.48 mg, 0.363 mmol, 0.9 equiv) and Ruphos (18.8 mg, 0.040 mmol, 0.1 equiv) and K₃PO₄ (256.61 mg, 1.209 mmol, 3 equiv) and 3rd Generation RuPhos precatalyst (16.85 mg, 0.020 mmol, 0.05 equiv) in 1,4-dioxane/H₂O(5:1.10 mL) was stirred for 4 h at 80° C. under nitrogen atmosphere. The mixture was allowed to cool down to room temperature. The reaction was quenched by the addition of Water (5 mL) at room temperature. The aqueous layer was extracted with EtOAc (3×10 mL). The resulting mixture was concentrated under reduced pressure. The residue was dissolved in CH₂Cl₂ (5 mL). The residue was purified by silica gel column chromatography, eluted with CH₂Cl₂/MeOH (7:1) to afford (3R)—N-tert-butyl-1-{6-[2-(methoxymethoxy)-4-(6-methoxypyridazin-4-yl)phenyl]pyridazin-3-yl}-N-methylpyrrolidin-3-amine (150 mg, 78%) as an oil. LCMS:(ES, m/z): 479.1 [M+H]⁺

### Synthesis of Compound 336

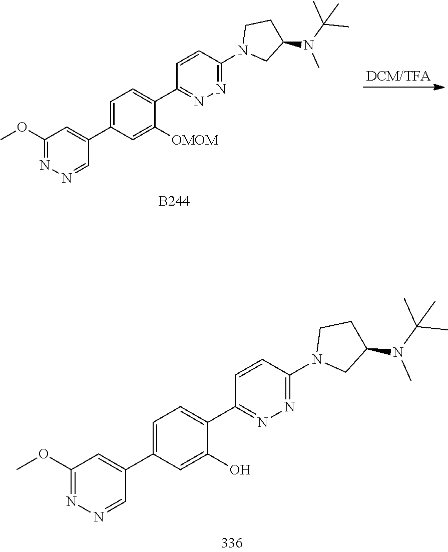

To a stirred solution of (3R)—N-tert-butyl-1-{6-[2-(methoxymethoxy)-4-(6-methoxypyridazin-4-yl) phenyl]pyridazin-3-yl}-N-methylpyrrolidin-3-amine (50 mg, 0.104 mmol, 1 equiv) in DCM(1 mL) was added TFA(0.5 mL) in portions at room temperature under air atmosphere.

Desired product could be detected by LCMS. The reaction was quenched with NH3 at 0° C. The crude product (100 mg) was purified by Prep-HPLC with the following conditions (Column, Kinetex EVO prep C18, 30*150, 5 um; mobile phase, Water (10 mmol/L NH₄HCO₃) and ACN (20% ACN up to 55% in 8 min)) to afford 2-{6-[(3R)-3-[tert-butyl(methyl)amino]pyrrolidin-1-yl]pyridazin-3-yl}-5-(6-methoxypyridazin-4-yl)phenol (22.1 mg, 49%) as a solid. LCMS:(ES, m/z):435.00 [M+H]^{+ 1}HNMR:(400 MHz, DMSO-d₆) δ 14.11 (s, 1H), 9.35 (d, J=1.9 Hz, 1H), 8.30 (d, J=9.8 Hz, 1H), 8.05 (d, J=8.3 Hz, 1H), 7.55 (d, J=1.9 Hz, 1H), 7.52-7.44 (m, 2H), 7.21 (d, J=9.8 Hz, 1H), 4.09 (s, 3H), 3.96 (t, J=8.4 Hz, 1H), 3.72 (t, J=9.8 Hz, 1H), 3.51 (t, J 9.2 Hz, 1H), 3.41 (dd, J=10.4, 7.2 Hz, 1H), 3.29 (s, 1H), 2.22 (s, 3H), 2.10-1.98 (m, 1H), 1.91 (m, 1H), 1.11 (s, 9H).

### Synthesis of Compound 337

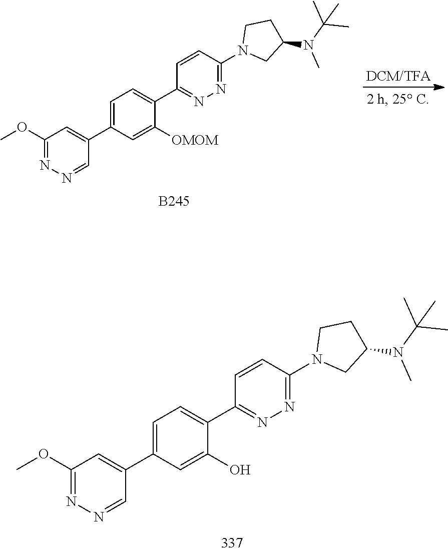

To a solution of (3S)—N-tert-butyl-1-{6-[2-(methoxymethoxy)-4-(6-methoxypyridazin-4-yl)phenyl]pyridazin-3-yl}-N-methylpyrrolidin-3-amine (100 mg, 0.209 mmol, 1 equiv) in DCM (2 mL) were added TFA (0.5 mL). After stirring for 2 h at 25° C. under a nitrogen atmosphere, the resulting mixture was concentrated under reduced pressure. The crude product was purified by Chiral-Prep-HPLC with the following conditions (2 #SHIMADZU (HPLC-01)): Column, XBridge Shield RP18 OBD Column, 30*150 mm, 5 μm; mobile phase, Water (10 mmol/L NH₄HCO₃) and ACN (20% ACN up to 55% in 10 min) to afford 2-{6-[(3S)-3-[tert-butyl(methyl)amino]pyrrolidin-1-yl]pyridazin-3-yl}-5-(6-methoxypyridazin-4-yl)phenol (18.5 mg, 20%) as a solid. LCMS:(ES, m/z):434[M+H]^{+ 1}HNMR: (400 MHz, DMSO-d₆) δ 14.10 (s, 1H), 9.34 (d, J=1.9 Hz, 1H), 8.30 (d, J=9.9 Hz, 1H), 8.04 (d, J=8.3 Hz, 1H), 7.55 (d, J=1.9 Hz, 1H), 7.50-7.43 (m, 2H), 7.21 (d, J=9.7 Hz, 1H), 4.09 (s, 3H), 3.96 (p, J=8.4 Hz, 1H), 3.72 (t, J=9.7 Hz, 1H), 3.51 (t, J=9.3 Hz, 1H), 3.41 (dd, J=10.4, 7.0 Hz, 1H), 3.35 (d, J=10.1 Hz, 1H), 2.22 (s, 3H), 2.11-1.98 (m, 1H), 1.93 (dt, J=12.9, 7.4 Hz, 1H), 1.11 (s, 9H).

### Example 97: Synthesis of Compounds 338 and 339

### Synthesis of Intermediate B245

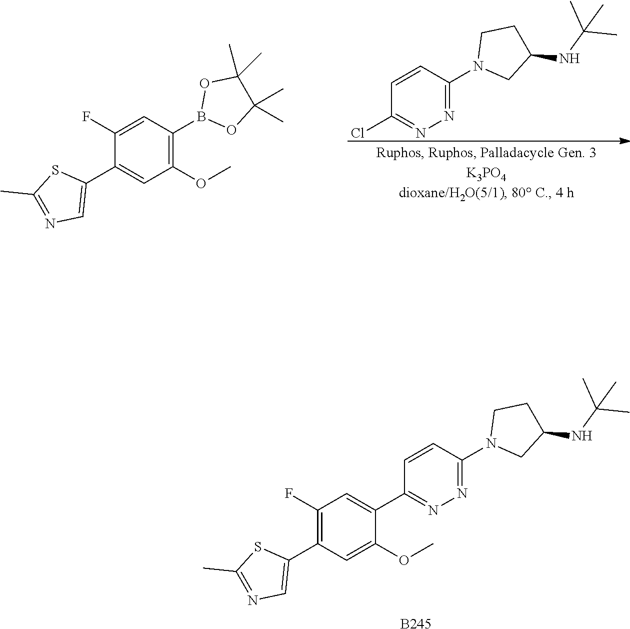

To a solution of 5-[2-fluoro-5-methoxy-4-(4,4,5,5-tetramethyl-1,3,2-dioxaborolan-2-yl) phenyl]-2-methyl-1,3-thiazole (450 mg, 1.289 mmol, 1 equiv) and (3R)—N-tert-butyl-1-(6-chloropyridazin-3-yl) pyrrolidin-3-amine (196.96 mg, 0.773 mmol, 0.6 equiv) in RuPhos (60.13 mg, 0.129 mmol, 0.1 equiv) were added K₃PO₄ (820.54 mg, 3.867 mmol, 3 equiv) and RuPhos Palladacycle Gen.3 (53.89 mg, 0.064 mmol, 0.05 equiv). After stirring for 4 h at 80° C. under a nitrogen atmosphere, the resulting mixture was concentrated under reduced pressure. The mixture was allowed to cool down to room temperature. The reaction was quenched by the addition of Water (10 mL) at room temperature. The aqueous layer was extracted with EtOAc (3×10 mL). The resulting mixture was concentrated under reduced pressure. The residue was dissolved in CH₂Cl₂ (5 mL). The residue was purified by silica gel column chromatography, eluted with CH₂Cl₂/MeOH (5:1) to afford (3R)—N-tert-butyl-1-{6-[5-fluoro-2-methoxy-4-(2-methyl-1,3-thiazol-5-yl) phenyl]pyridazin-3-yl}pyrrolidin-3-amine (230 mg, 40%) as a solid. LCMS:(ES, m/z):441.20 [M+H]⁺

### Synthesis of Compound 338

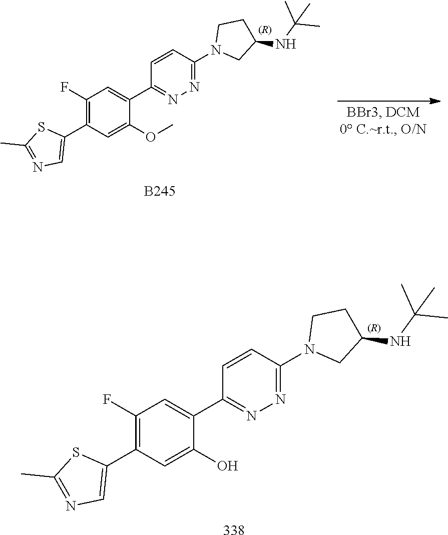

A mixture of (3R)—N-tert-butyl-1-{6-[5-fluoro-2-methoxy-4-(2-methyl-1,3-thiazol-5-yl) phenyl]pyridazin-3-yl}pyrrolidin-3-amine (25 mg, 0.057 mmol, 1 equiv) and BBr₃ (0.1 mL, 1.058 mmol, 18.68 equiv) in DCM (1 mL) was stirred for 16 h at 0-25° C. The reaction was quenched with MeOH at 0° C. The mixture was basified to pH 10 with amino methanol. The resulting mixture was washed with 2×1 20 mL of water. The resulting mixture was concentrated under reduced pressure. The crude product (100 mg) was purified by Prep-HPLC with the following conditions (Column, XBridge Shield RP18 OBD Column, 30×150 mm, 5 μm; mobile phase, Water (10 mmol/L NH₄HCO₃) and ACN (20% ACN up to 60% in 8 min)) to afford 2-{6-[(3R)-3-(tert-butylamino) pyrrolidin-1-yl]pyridazin-3-yl}-4-fluoro-5-(2-methyl-1,3-thiazol-5-yl) phenol (12.9 mg, 7%) as a solid. LCMS:(ES, m/z):427.90 [M+H]^{+ 1}H NMR: (400 MHz, DMSO-d₆) δ 13.74 (s, 1H), 8.25 (d, J=9.8 Hz, 1H), 8.16 (s, 1H), 7.93 (d, J=12.6 Hz, 1H), 7.29 (d, J=6.8 Hz, 1H), 7.17 (d, J=9.7 Hz, 1H), 3.79 (d, J=9.6 Hz, 1H), 3.65 (s, 1H), 3.54 (s, 2H), 3.44 (d, J=9.0 Hz, 1H), 3.09 (s, 1H), 2.71 (s, 3H), 2.21-2.14 (m, 1H), 1.77 (t, J=10.1 Hz, 1H), 1.24 (s, 1H), 1.09 (s, 9H). ¹⁹F NMR: (376 MHz, DMSO-d₆) δ −125.46.

### Synthesis of Compound 339

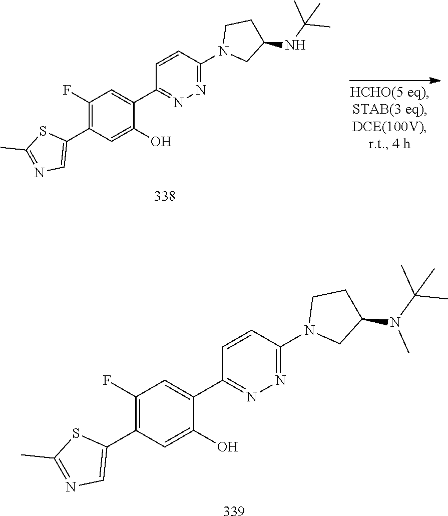

A solution of 2-{6-[(3R)-3-(tert-butylamino)pyrrolidin-1-yl]pyridazin-3-yl}-4-fluoro-5-(2-methyl-1,3-thiazol-5-yl)phenol (120 mg, 0.281 mmol, 1 equiv) and HCHO (42.14 mg, 1.405 mmol, 5 equiv) and STAB (178.46 mg, 0.843 mmol, 3 equiv) in DCE(1 mL) was stirred for 4 h at 25° C. The crude product (30 mg) was purified by Prep-HPLC with the following conditions (Column, CHIRALPAK IA, 2×25 cm, 5 um; mobile phase, MtBE 0.1% DEA) and MeOH(hold 50% MeOH-in 9 min)) to afford 2-{6-[(3R)-3-[tert-butyl(methyl)amino]pyrrolidin-1-yl]pyridazin-3-yl}-4-fluoro-5-(2-methyl-1,3-thiazol-5-yl)phenol (4.9 mg, 4%) as a solid. LCMS:(ES, m/z):441.90[M+H]^{+ 1}H NMR: (400 MHz, DMSO-d₆) δ 13.74 (s, 1H), 8.26 (d, J=9.8 Hz, 1H), 8.16 (s, 1H), 7.94 (d, J=12.7 Hz, 1H), 7.30 (d, J=6.8 Hz, 1H), 7.20 (d, J=9.7 Hz, 1H), 3.95 (q, J=8.6 Hz, 1H), 3.70 (d, J=9.7 Hz, 1H), 3.50 (t, J=9.3 Hz, 1H), 3.39 (d, J=7.8 Hz, 1H), 3.37 (d, J=7.6 Hz, 1H), 2.71 (s, 3H), 2.21 (s, 1H), 1.93 (q, J=6.4 Hz, 1H), 1.10 (s, 9H). ¹⁹F NMR: (376 MHz, DMSO-d₆) δ −125.44.

### Example 98: Synthesis of Compound 342

### Synthesis of Intermediate B246

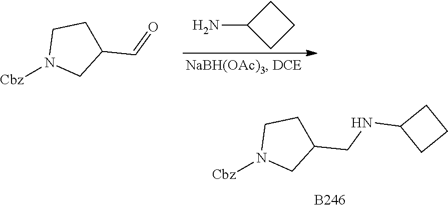 [SMILES: "B246" ; O=CC1CCN(C(=O)OCc2ccccc2)C1 ; NC1CCC1 ; O=C(OCc1ccccc1)N1CCC(CNC2CCC2)C1]

A solution of benzyl 3-formylpyrrolidine-1-carboxylate (5 g, 21.435 mmol, 1 equiv) and cyclobutylamine (1.83 g, 25.722 mmol, 1.2 equiv) and STAB (13.63 g, 64.305 mmol, 3 equiv) in DCE (100 mL) was stirred for 4 h at 25° C. The reaction was quenched by the addition of water (100 mL) at room temperature. The aqueous layer was extracted with EtOAc (3×100 mL). The resulting mixture was concentrated under reduced pressure. The residue was dissolved in CH₂Cl₂ (20 mL). The residue was purified by silica gel column chromatography, eluted with CH₂Cl₂/MeOH (6:1) to afford benzyl 3-[(cyclobutylamino)methyl]pyrrolidine-1-carboxylate (7.5 g, 121%) as an oil. LCMS:(ES, m/z):288.90[M+H]⁺

### Synthesis of Intermediate B247

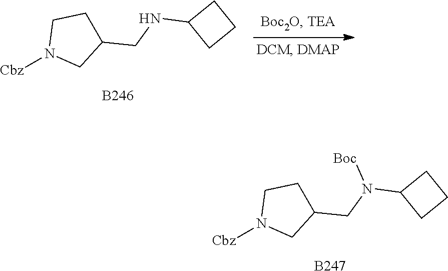 [SMILES: "B246" ; "B247" ; O=C(OCc1ccccc1)N1CCC(CNC2CCC2)C1 ; CC(C)(C)OC(=O)N(CC1CCN(C(=O)OCc2ccccc2)C1)C1CCC1]

A solution of benzyl 3-[(cyclobutylamino)methyl]pyrrolidine-1-carboxylate (5 g, 17.338 mmol, 1 equiv) and Boc₂O (7.57 g, 34.676 mmol, 2 equiv) and DIEA (4.48 g, 34.676 mmol, 2 equiv) in DCM (100 mL) was stirred for 4 h at 25° C. The mixture was allowed to cool down to room temperature. The reaction was quenched by the addition of Water (100 mL) at room temperature. The aqueous layer was extracted with CH₂Cl₂ (2×100 mL). The resulting mixture was concentrated under reduced pressure. The residue was dissolved in CH₂Cl₂ (20 mL). The residue was purified by silica gel column chromatography, eluted with CH₂Cl₂/MeOH (5:1) to afford benzyl 3-{[(tert-butoxycarbonyl) (cyclobutyl)amino]methyl}pyrrolidine-1-carboxylate (4.5 g, 67%) as a solid. LCMS:(ES, m/z):388.20[M+H]⁺

### Synthesis of Intermediate B248

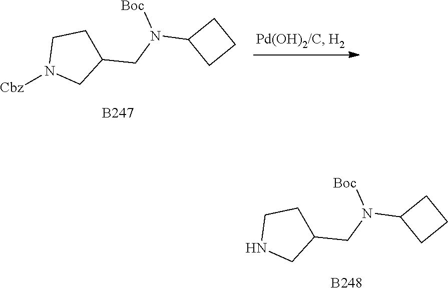 [SMILES: "B247" ; "B248" ; CC(C)(C)OC(=O)N(CC1CCN(C(=O)OCc2ccccc2)C1)C1CCC1 ; CC(C)(C)OC(=O)N(CC1CCNC1)C1CCC1]

A mixture of benzyl 3-{[(tert-butoxycarbonyl) (cyclobutyl)amino]methyl}pyrrolidine-1-carboxylate (3 g, 7.722 mmol, 1 equiv) and Pd(OH)₂/C (0.9 g, 6.409 mmol, 0.83 equiv) in MeOH (30 mL) was stirred for 6 h at room temperature under hydrogen atmosphere. The resulting mixture was filtered and the filter cake was washed with MeOH (2×30 mL). The filtrate was concentrated under reduced pressure. The residue was purified by silica gel column chromatography, eluted with CH₂Cl₂/MeOH (5:1) to afford tert-butyl N-cyclobutyl-N-(pyrrolidin-3-ylmethyl)carbamate (1.7 g, 87%) as a solid.

### Synthesis of Intermediate B249

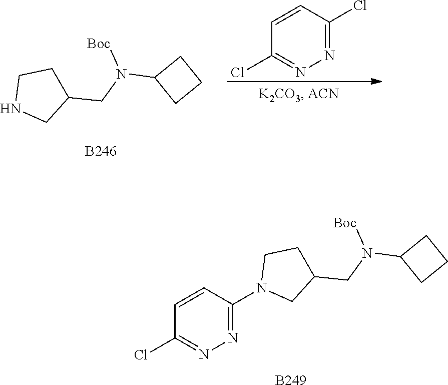

A solution of tert-butyl N-cyclobutyl-N-(pyrrolidin-3-ylmethyl) carbamate (1.7 g, 6.683 mmol, 1 equiv) in ACN (20 mL) was treated with pyridazine, 3,6-dichloro- (1.00 g, 6.683 mmol, 1 equiv) for 4 h at 80° C. The mixture was allowed to cool down to room temperature. The reaction was quenched by the addition of Water (5 mL) at room temperature. The aqueous layer was extracted with EtOAc (3×10 mL). The resulting mixture was concentrated under reduced pressure. The residue was purified by silica gel column chromatography, eluted with CH₂Cl₂/MeOH (5:1) to afford N-{[1-(6-chloropyridazin-3-yl)pyrrolidin-3-yl]methyl}cyclobutanamine (1.3 g, 72.92%) as an oil. LCMS:(ES, m/z):367.05[M+H]⁺

### Synthesis of Intermediate B250

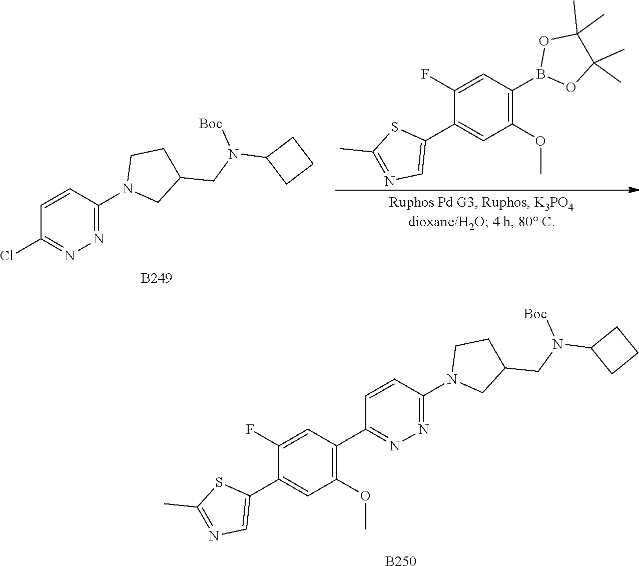

To a solution of tert-butyl N-{[1-(6-chloropyridazin-3-yl)pyrrolidin-3-yl]methyl}-N-cyclobutylcarbamate (84.05 mg, 0.229 mmol, 0.8 equiv) and 5-[2-fluoro-5-methoxy-4-(4,4,5,5-tetramethyl-1,3,2-dioxaborolan-2-yl)phenyl]-2-methyl-1,3-thiazole (100 mg, 0.286 mmol, 1.00 equiv) in dioxane (5.0 mL) and water (1.0 mL) were added K₃PO₄ (182.34 mg, 0.858 mmol, 3 equiv) and 3rd Generation RuPhos precatalyst (11.97 mg, 0.014 mmol, 0.05 equiv) and Ruphos (13.36 mg, 0.029 mmol, 0.1 equiv). After stirring for 4 h at 80° C. under a nitrogen atmosphere, the mixture was allowed to cool down to room temperature, the resulting mixture was concentrated under reduced pressure. The residue was purified by Prep-TLC column chromatography, eluted with CH₂Cl₂/MeOH (5:1) to afford tert-butyl N-cyclobutyl-N-[(1-{6-[5-fluoro-2-methoxy-4-(2-methyl-1,3-thiazol-5-yl)phenyl]pyridazin-3-yl}pyrrolidin-3-yl)methyl]carbamate) as a solid. LCMS:(ES, m/z):554.05[M+H]⁺

### Synthesis of Compound 342

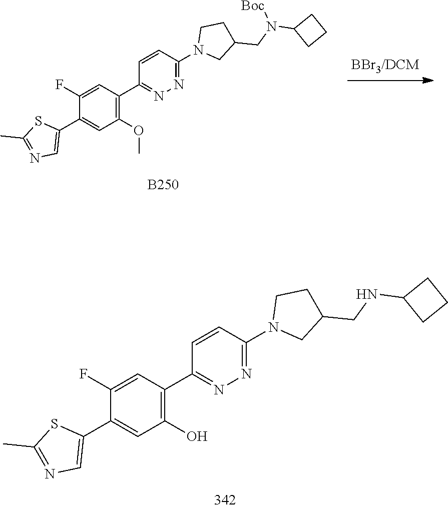 [SMILES: "B250" ; COc1cc(-c2cnc(C)s2)c(F)cc1-c1ccc(N2CCC(CN(C(=O)OC(C)(C)C)C3CCC3)C2)nn1 ; Cc1ncc(-c2cc(O)c(-c3ccc(N4CCC(CNC5CCC5)C4)nn3)cc2F)s1]

To a stirred mixture of tert-butyl N-cyclobutyl-N-[(1-{6-[5-fluoro-2-methoxy-4-(2-methyl-1,3-thiazol-5-yl)phenyl]pyridazin-3-yl}pyrrolidin-3-yl)methyl]carbamate (50 mg, 0.090 mmol, 1 equiv) in DCM (1 mL) was added BBr₃ (0.04 mL, 0.450 mmol, 5 equiv) dropwise at 0° C. under air atmosphere. The reaction was quenched by the addition of MeOH (5 mL) at 0° C. The resulting mixture was concentrated under reduced pressure. The mixture was basified to pH 8 with NH₃·H₂O. The resulting mixture was extracted with EtOAc (2×30 mL), dried over anhydrous Na₂SO₄. After filtration, the filtrate was concentrated under reduced pressure. The crude product (50 mg) was purified by Prep-HPLC with the following conditions (Column, Kinetex EVO prep C18, 30×150, 5 um; mobile phase, Water (10 mmol/L NH₄HCO₃) and ACN (20% ACN up to 55% in 8 min)) to afford 2-(6-{3-[(cyclobutylamino)methyl]pyrrolidin-1-yl}pyridazin-3-yl)-4-fluoro-5-(2-methyl-1,3-thiazol-5-yl)phenol (4.6 mg, 12%) as a solid. LCMS:(ES, m/z):439.90 [M+H]⁺¹H NMR:(400 MHz, DMSO-d₆) δ 13.74 (s, 1H), 8.26 (d, J=9.8 Hz, 1H), 8.16 (s, 1H), 7.93 (d, J=12.7 Hz, 1H), 7.29 (d, J=6.8 Hz, 1H), 7.17 (d, J=9.8 Hz, 1H), 3.71-3.60 (m, 2H), 3.49 (d, J=9.3 Hz, 1H), 3.29 (s, 1H), 3.19 (s, 2H), 2.71 (s, 3H), 2.41 (s, 1H), 2.10 (d, J=7.8 Hz, 1H), 1.75 (dt, J=23.1, 8.9 Hz, 3H), 1.68-1.53 (m, 5H). ¹⁹F NMR: (376 MHz, DMSO-d₆) δ −37.17, −125.45.

### Example 99: Synthesis of Compound 343

### Synthesis of Intermediate B251

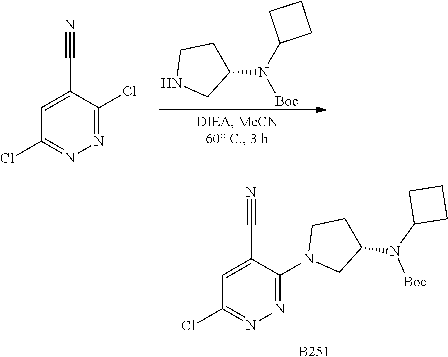 [SMILES: "B251" ; N#Cc1cc(Cl)nnc1Cl ; CC(C)(C)OC(=O)N(C1CCC1)[C@H]1CCNC1 ; CC(C)(C)OC(=O)N(C1CCC1)[C@H]1CCN(c2nnc(Cl)cc2C#N)C1]

A solution of 3,6-dichloropyridazine-4-carbonitrile (1 g, 5.748 mmol, 1 equiv) in MeCN (10 mL) was treated with tert-butyl N-cyclobutyl-N-[(3S)-pyrrolidin-3-yl]carbamate (1.38 g, 5.748 mmol, 1 equiv) and DIEA (1.49 g, 11.496 mmol, 2 equiv) for 3 h at 60° C. LCMS showed the obtaining of the desired Ms. The mixture was washed with water and extracted with EtOAc (100 mL). The organic layer was washed with brine (100 mL), dried over Na₂SO₄ and concentrated.

The crude was purified by column chromatograph on silica gel (PE:EA=10:1-3:1) to give B251 (1.4 g, 64.46% yield). LCMS:(ES, m/z):378.0 [M+H]⁺¹H NMR: (400 MHz, DMSO-d₆) δ 8.19 (s, 1H), 4.46-4.41 (m, 1H), 4.28-4.05 (m, 1H), 3.98-3.93 (m, 1H), 3.85-3.81 (m, 2H), 3.72-3.71 (m, 1H), 2.49-2.35 (m, 3H), 2.10-2.06 (m, 3H), 1.63-1.53 (m, 2H), 1.41 (s, 9H).

### Synthesis of Intermediate B252

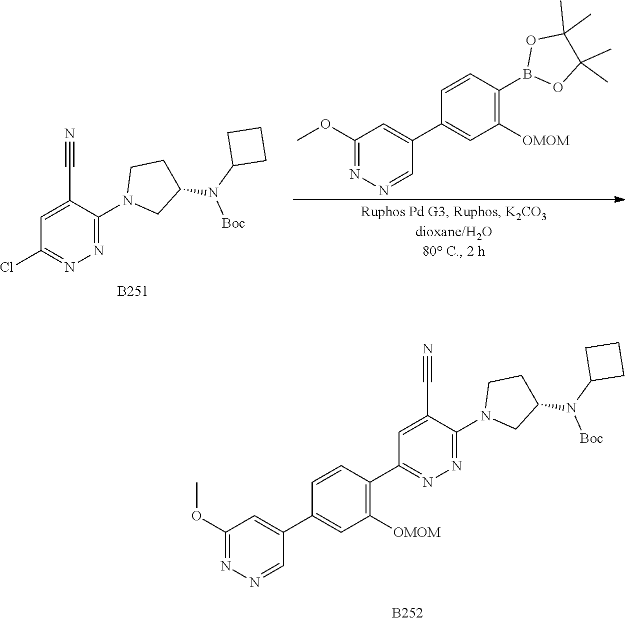

A solution of tert-butyl N-[(3S)-1-(6-chloro-4-methylpyridazin-3-yl)pyrrolidin-3-yl]-N-cyclobutylcarbamate (140 mg, 0.382 mmol, 1 equiv), 3-methoxy-5-[3-(methoxymethoxy)-4-(4,4,5,5-tetramethyl-1,3,2-dioxaborolan-2-yl)phenyl]pyridazine (142.04 mg, 0.382 mmol, 1 equiv), K₂CO₃ (158.21 mg, 1.146 mmol, 3 equiv), 3rd Generation RuPhos precatalyst (31.92 mg, 0.038 mmol, 0.1 equiv), RuPhos (35.61 mg, 0.076 mmol, 0.2 equiv) in 1,4-dioxane and water was degassed and purged with nitrogen atmosphere. Then the mixture was stirred at 80° C. for 2 h under nitrogen atmosphere. The mixture was filtered and the filtrate was concentrated. The crude was purified by prep-TLC (EtOAc) to give tert-butyl N-cyclobutyl-N-[(3S)-1-{6-[2-(methoxymethoxy)-4-(6-methoxypyridazin-4-yl)phenyl]-4-methylpyridazin-3-yl}pyrrolidin-3-yl]carbamate (60 mg, 20%). LCMS:(ES, m/z): 588.1[M+H]^{+ 1}HNMR: (400 MHz, DMSO-d₆) δ 9.37 (s, 1H), 8.23 (s, 1H), 7.92 (d, J=8.0 Hz, 1H), 7.72-7.71 (m, 2H), 7.60 (s, 1H), 5.44 (s, 2H), 4.53-4.44 (m, 1H), 4.36-4.29 (m, 1H), 4.27 (s, 3H), 3.92 (m, 2H), 3.82-3.78 (m, 1H), 3.31 (s, 3H), 2.42-2.28 (m, 3H), 2.15-2.12 (m, 3H), 1.65-1.55 (m, 2H), 1.43 (s, 9H).

### Synthesis of Compound 343

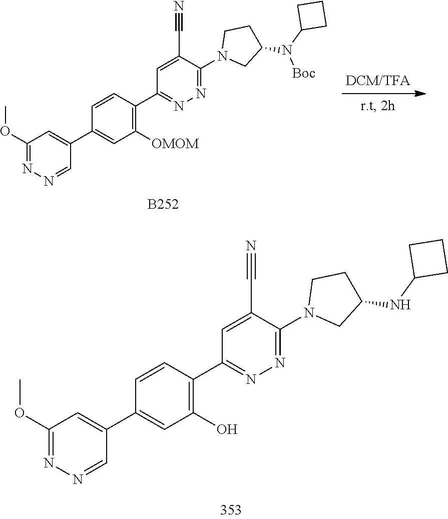

A mixture of tert-butyl N-[(3S)-1-{4-cyano-6-[2-(methoxymethoxy)-4-(6-methoxypyridazin-4-yl)phenyl]pyridazin-3-yl}pyrrolidin-3-yl]-N-cyclobutylcarbamate (60 mg, 0.102 mmol, 1 equiv) and TFA (1 mL) in DCM (5 mL) was stirred at room temperature for 2hThe mixture was concentrated and the crude was purified by prep-HPLC (condition: (2 #SHIMADZU (HPLC-01)): Column, Kinetex EVO prep C18, 30*150, 5 um; mobile phase, Water (10 mmol/L NH₄HCO₃) and ACN (20% ACN up to 55% in 10 min); Detector, UV) and Chiral-Prep-HPLC with the following conditions (2 #SHIMADZU (HPLC-01)): Column, CHIRALPAK IC, 2*25 cm, 5 um; mobile phase, MtBE(0.1% DEA) and MeOH- (hold 50% MeOH- in 26 min); Detector, UV) to give 3-[(3S)-3-(cyclobutylamino)pyrrolidin-1-yl]-6-[2-hydroxy-4-(6-methoxypyridazin-4-yl)phenyl]pyridazine-4-carbonitrile (21.0 mg, 46% yield). LCMS:(ES, m/z): 443.95[M+H]⁺¹HNMR: (400 MHz, DMSO-d₆) δ 12.32 (s, 1H), 9.32 (s, 1H), 8.70 (s, 1H), 8.07 (d, J=8.0 Hz, 1H), 7.53-7.49 (m, 3H), 4.09 (s, 3H), 3.94-3.92 (m, 2H), 3.91-3.90 (m, 1H), 3.83-3.81 (m, 1H), 3.60-3.58 (m, 1H), 3.32 (m, 1H), 2.15-2.09 (m, 3H), 1.88-1.86 (m, 1H), 1.74 (m, 2H), 1.66-1.60 (m, 2H).

### Example 100: Synthesis of Compound 344

### Synthesis of Intermediate B253

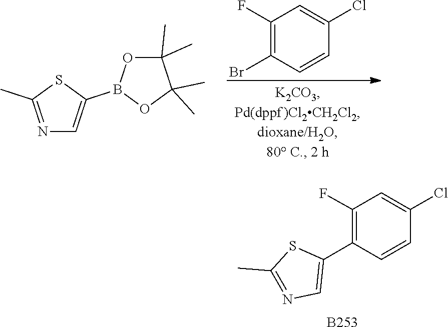

A solution of 2-methyl-5-(4,4,5,5-tetramethyl-1,3,2-dioxaborolan-2-yl)-1,3-thiazole (1 g, 4.442 mmol, 1 equiv) in 1,4-dioxane and H₂O was treated with 1-bromo-4-chloro-2-fluorobenzene (837.35 mg, 3.998 mmol, 0.9 equiv), K₂CO₃ (1.23 g, 8.884 mmol, 2 equiv) and Pd(dppf)Cl₂·CH₂Cl₂ (36.19 mg, 0.044 mmol, 0.1 equiv) for 2 h at 80° C. under nitrogen atmosphere. LCMS showed the obtaining of the product. The mixture was extracted with EtOAc (50 mL) and washed with water and brine, dried over with Na₂SO₄ and concentrated. The crude was purified by column chromatograph on silica gel (0˜30% EtOAc in PE) to give product.

LCMS:(ES, m/z): 228 [M+H]⁺

### Synthesis of Intermediate B254

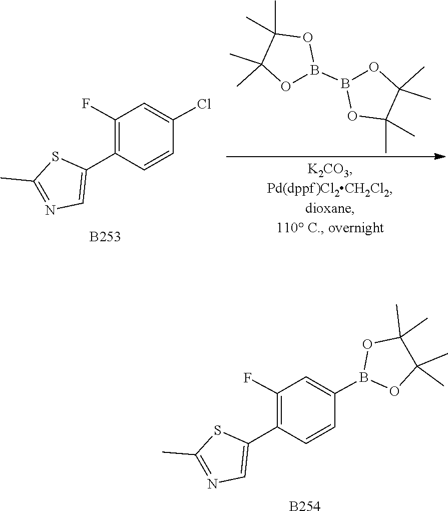

A solution of 5-(4-chloro-2-fluorophenyl)-2-methyl-1,3-thiazole (190 mg, 0.835 mmol, 1 equiv), bis(pinacolato)diboron (254.3 mg, 1.002 mmol, 1.2 equiv), KOAc (163.8 mg, 1.670 mmol, 2 equiv), Pd(dppf)Cl₂·CH₂Cl₂ (33.99 mg, 0.042 mmol, 0.05 equiv) in 1,4-dioxane was stirred at 80° C. for 2 h. LCMS showed no reaction. The mixture was stirred at 110° C. overnight. The mixture was filtered and the crude was used directly. LCMS:(ES, m/z): 320 [M+H]⁺

### Synthesis of Intermediate B255

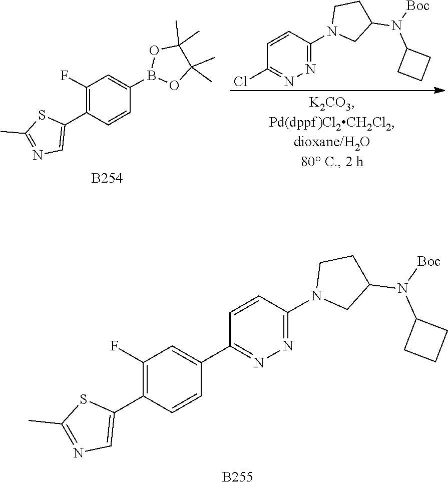

A solution of tert-butyl N-[1-(6-chloropyridazin-3-yl)pyrrolidin-3-yl]-N-cyclobutylcarbamate (200 mg, 0.567 mmol, 1 equiv), 5-[2-fluoro-4-(4,4,5,5-tetramethyl-1,3,2-dioxaborolan-2-yl)phenyl]-2-methyl-1,3-thiazole (300 mg, 0.564 mmol, 0.99 equiv, 60%), K₂CO₃ (235.0 mg, 1.701 mmol, 3 equiv), Pd(dppf)Cl₂·CH₂Cl₂ (23.09 mg, 0.028 mmol, 0.05 equiv) in dioxane and water was stirred at 80° C. for 2 h. LCMS showed the obtaining of the desired Ms. The mixture was extracted with EtOAc (50 mL) and washed with water and brine, dried over with Na₂SO₄ and concentrated. The crude was purified by prep-TLC (PE:EA=1:2) to give product.

LCMS:(ES, m/z): 510 [M+H]⁺

### Synthesis of Compound 344

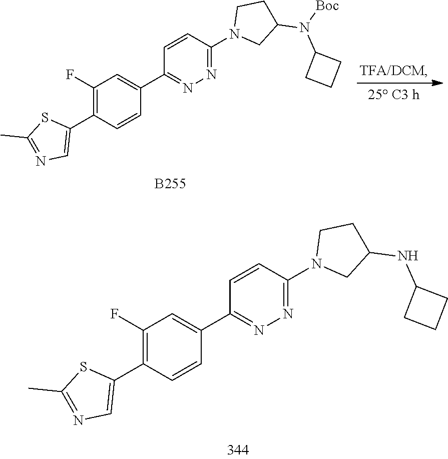 [SMILES: "B255" ; Cc1ncc(-c2ccc(-c3ccc(N4CCC(N(C(=O)OC(C)(C)C)C5CCC5)C4)nn3)cc2F)s1 ; Cc1ncc(-c2ccc(-c3ccc(N4CCC(NC5CCC5)C4)nn3)cc2F)s1]

A solution of tert-butyl N-cyclobutyl-N-(1-{6-[3-fluoro-4-(2-methyl-1,3-thiazol-5-yl)phenyl]pyridazin-3-yl}pyrrolidin-3-yl)carbamate, 1 equiv, 30%) in DCM (10 mL) and TFA (2 mL) was stirred at 25° C. for 3h. LCMS showed the reaction was completed. The mixture was concentrated and the crude was purified by prep-HPLC with the following conditions (2 #SHIMADZU (HPLC-01)): Column, Kinetex EVO prep C18, 30*150, 5 um; mobile phase, Water (10 mmol/L NH₄HCO₃) and ACN (15% ACN up to 60% in 10 min); Detector, UV) to give product. LCMS:(ES, m/z): 410 [M+H]^{+ 1}H-NMR: (400 MHz, DMSO-d₆) δ 8.16 (s, 1H), 7.99 (dd, J=14.8, 9.3 Hz, 3H), 7.88 (t, J=8.0 Hz, 1H), 6.94 (d, J=9.6 Hz, 1H), 3.61 (d, J=16.1 Hz, 2H), 3.49 (s, 1H), 3.39-3.35 (m, 1H), 3.30 (s, 1H), 3.27-3.23 (m, 1H), 2.72 (s, 3H), 2.17-2.06 (m, 3H), 1.81 (dd, J=12.6, 6.4 Hz, 1H), 1.69 (d, J=10.1 Hz, 2H), 1.66-1.49 (m, 2H).

### Example 101: Synthesis of Compound 346

### Synthesis of Intermediate B256

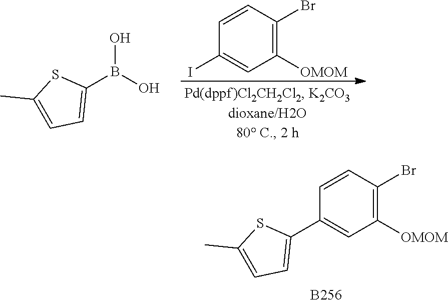

To a solution of (5-methylthiophen-2-yl)boronic acid (1 g, 7.043 mmol, 1 equiv) and 1-bromo-4-iodo-2-(methoxymethoxy)benzene (2.42 g, 7.043 mmol, 1 equiv) in 1,4-dioxane (2 mL) and H₂O (0.5 mL) were added K₂CO₃ (2.43 g, 17.608 mmol, 2.5 equiv) and Pd(dppf)Cl₂CH₂Cl₂ (0.29 g, 0.352 mmol, 0.05 equiv). After stirring for 2 h at 80° C. under a nitrogen atmosphere, the resulting mixture was concentrated under reduced pressure. The residue was purified by Prep-TLC/silica gel column chromatography, eluted with PE/EA (1:1) to afford 2-(4-bromo-3-(methoxymethoxy)phenyl)-5-methylthiophene (500 mg, 23%) as a solid.

### Synthesis of Intermediate B257

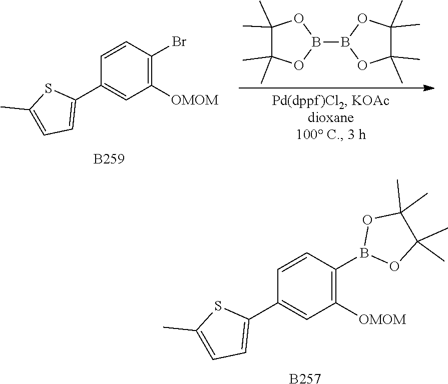

To a solution of 2-(4-bromo-3-(methoxymethoxy)phenyl)-5-methylthiophene (500 mg, 1.596 mmol, 1 equiv) and bis(pinacolato)diboron (608.07 mg, 2.394 mmol, 1.5 equiv) in 1,4-dioxane (3 mL) were added KOAc (470.01 mg, 4.788 mmol, 3 equiv) and Pd(dppf)Cl₂ (58.4 mg, 0.080 mmol, 0.05 equiv). After stirring for 3 h at 100° C. under a nitrogen atmosphere. The resulting mixture was filtered and the filter cake was washed with DCM (3×10 mL). The filtrate was concentrated under reduced pressure. to afford crude 2-(2-(methoxymethoxy)-4-(5-methylthiophen-2-yl)phenyl)-4,4,5,5-tetramethyl-1,3,2-dioxaborolane (300 mg) as a brown oil.

### Synthesis of Intermediate B258

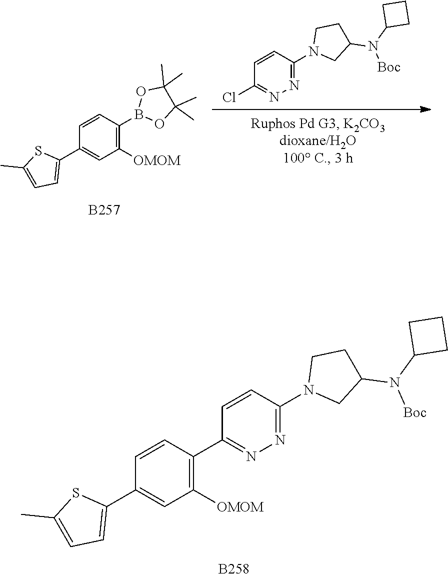

To a solution of 2-(2-(methoxymethoxy)-4-(5-methylthiophen-2-yl)phenyl)-4,4,5,5-tetramethyl-1,3,2-dioxaborolane (300 mg, 0.833 mmol, 1 equiv) and tert-butyl N-[1-(6-chloropyridazin-3-yl)pyrrolidin-3-yl]-N-cyclobutylcarbamate (323.2 mg, 0.916 mmol, 1.1 equiv) in 1,4-dioxane (2 mL) and H₂O (0.5 mL) were added K₂CO₃ (345.24 mg, 2.499 mmol, 3 equiv) and RuPhos Palladacycle Gen.3 (69.64 mg, 0.083 mmol, 0.1 equiv). After stirring for 3 h at 100° C. under a nitrogen atmosphere, the resulting mixture was concentrated under reduced pressure. The residue was purified by Prep-TLC/silica gel column chromatography, eluted with CHCl₃/MeOH (10:1) to afford tert-butyl cyclobutyl(1-(6-(2-(methoxymethoxy)-4-(5-methylthiophen-2-yl)phenyl)pyridazin-3-yl)pyrrolidin-3-yl)carbamate (30 mg) as a brown solid.

### Synthesis of Compound 346

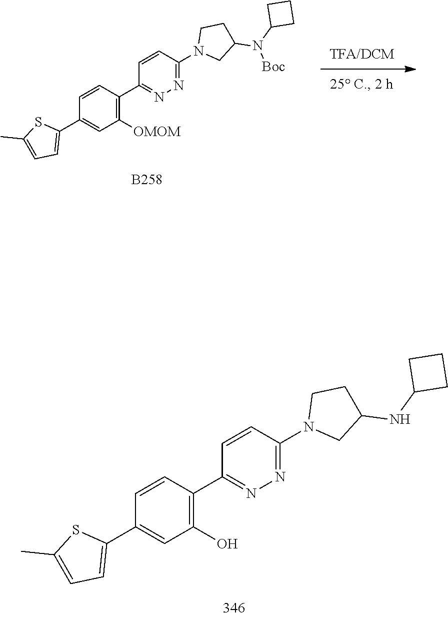

To a solution of tert-butyl N-cyclobutyl-N-(1-{6-[2-(methoxymethoxy)-4-(5-methylthiophen-2-yl)phenyl]pyridazin-3-yl}pyrrolidin-3-yl)carbamate (30 mg, 0.054 mmol, 1 equiv) in DCM (1 mL, 15.731 mmol, 288.77 equiv) were added TFA (0.3 mL, 4.039 mmol, 74.14 equiv) at 0° C.

After stirring for 0.5 h at 25° C. under a nitrogen atmosphere, the resulting mixture was concentrated under reduced pressure. The crude product was purified by Chiral-Prep-HPLC with the following conditions (2 #SHIMADZU (HPLC-01)): Column, XBridge Shield RP18 OBD Column, 30*150 mm, 5 μm; mobile phase, Water (10 mmol/L NH₄HCO₃) and ACN (30% ACN up to 75% in 10 min) to afford 2-{6-[3-(cyclobutylamino)pyrrolidin-1-yl]pyridazin-3-yl}-5-(5-methylthiophen-2-yl)phenol (2.1 mg, 9%) as a solid. LCMS (ES, m/z): 406.9 [M+H]^{+ 1}H NMR (400 MHz, DMSO-d₆) δ 13.95 (s, 1H), 8.19 (d, J=9.8 Hz, 1H), 7.87 (d, J=8.2 Hz, 1H), 7.38 (d, J=3.5 Hz, 1H), 7.18-7.14 (m, 1H), 7.12 (d, J=2.9 Hz, 2H), 6.84 (dd, J=3.6, 1.2 Hz, 1H), 3.74-3.55 (m, 2H), 3.53-3.45 (m, 1H), 3.38 (d, J=5.6 Hz, 1H), 3.30 (s, 1H), 3.23 (q, J=8.2 Hz, 1H), 2.49-2.47 (m, 3H), 2.23-2.00 (m, 4H), 1.81 (dq, J=13.1, 6.7 Hz, 1H), 1.74-1.62 (m, 2H), 1.62-1.48 (m, 2H).

### Example 102: Synthesis of Compound 347

### Synthesis of Intermediate B259

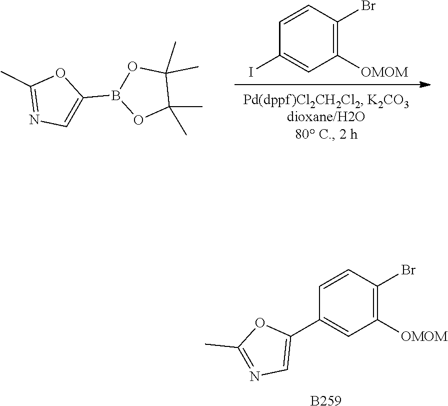

To a solution of 2-methyl-5-(4,4,5,5-tetramethyl-1,3,2-dioxaborolan-2-yl)oxazole (1.659 g, 7.942 mmol, 1 equiv) and 1-bromo-4-iodo-2-(methoxymethoxy)benzene (2.72 g, 7.942 mmol, 1 equiv) in 1,4-dioxane (2 mL) and H₂O (0.5 mL) were added K₂CO₃ (2.74 g, 19.855 mmol, 2.5 equiv) and Pd(dppf)Cl₂CH₂Cl₂ (0.32 g, 0.397 mmol, 0.05 equiv). After stirring for 2 h at 80° C. under a nitrogen atmosphere, the resulting mixture was concentrated under reduced pressure. The residue was purified by silica gel column chromatography, eluted with PE/EA (1:1) to afford 5-(4-bromo-3-(methoxymethoxy)phenyl)-2-methyloxazole (600 mg, 24%) as a solid.

### Synthesis of Intermediate B260

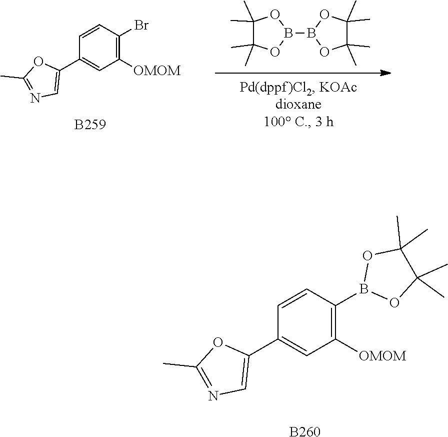

To a solution of 5-(4-bromo-3-(methoxymethoxy)phenyl)-2-methyloxazole (600 mg, 2.02 mmol, 1 equiv) and bis(pinacolato)diboron (769 mg, 3.03 mmol, 1.5 equiv) in 1,4-dioxane (3 mL) were added KOAc (594.79 mg, 6.060 mmol, 3 equiv) and Pd(dppf)Cl₂ (73.83 mg, 0.101 mmol, 0.05 equiv). After stirring for 3 h at 100° C. under a nitrogen atmosphere. The resulting mixture was filtered and the filter cake was washed with DCM (3×10 mL). The filtrate was concentrated under reduced pressure. to afford 5-(3-(methoxymethoxy)-4-(4,4,5,5-tetramethyl-1,3,2-dioxaborolan-2-yl)phenyl)-2-methyloxazole (600 mg, 86%) as an oil.

### Synthesis of Intermediate B261

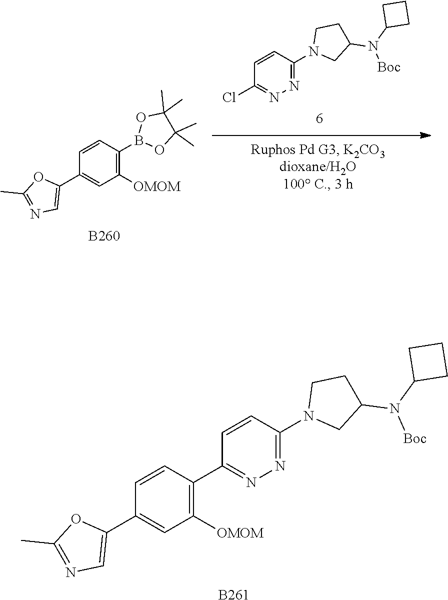

To a solution of 5-(3-(methoxymethoxy)-4-(4,4,5,5-tetramethyl-1,3,2-dioxaborolan-2-yl)phenyl)-2-methyloxazole (300 mg, 0.869 mmol, 1 equiv) and tert-butyl N-[1-(6-chloropyridazin-3-yl)pyrrolidin-3-yl]-N-cyclobutylcarbamate (337.23 mg, 0.956 mmol, 1.1 equiv) in 1,4-dioxane (2 mL) and H₂O (0.5 mL) were added K₂CO₃ (360.12 mg, 2.607 mmol, 3 equiv) and RuPhos Palladacycle Gen.3 (72.9 mg, 0.087 mmol, 0.1 equiv). After stirring for 3 h at 100° C. under a nitrogen atmosphere, the resulting mixture was concentrated under reduced pressure. The residue was purified by silica gel column chromatography, eluted with CHCl₃/MeOH (10:1) to afford tert-butyl cyclobutyl(1-(6-(2-(methoxymethoxy)-4-(2-methyloxazol-5-yl)phenyl)pyridazin-3-yl)pyrrolidin-3-yl)carbamate (50 mg, 11%) as a solid.

### Synthesis of Compound 347

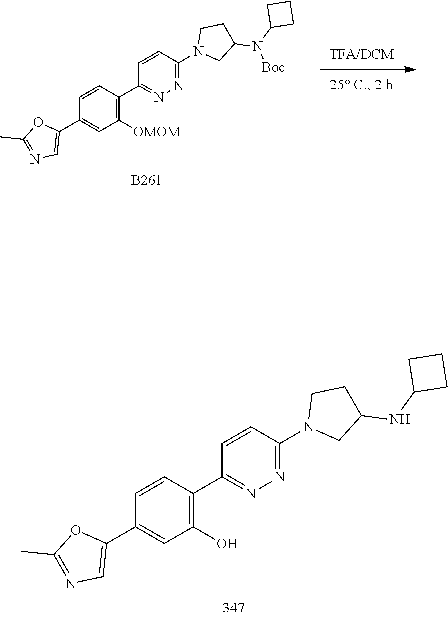

To a solution of tert-butyl N-cyclobutyl-N-(1-{6-[2-(methoxymethoxy)-4-(2-methyl-1,3-oxazol-5-yl)phenyl]pyridazin-3-yl}pyrrolidin-3-yl)carbamate (50 mg, 0.093 mmol, 1 equiv) in DCM (1 mL, 15.731 mmol, 168.52 equiv) were added TFA (0.4 mL, 5.385 mmol, 57.69 equiv).

After stirring for 1 h at 25° C. under a nitrogen atmosphere, the resulting mixture was concentrated under reduced pressure. The crude product was purified by Chiral-Prep-HPLC with the following conditions (2 #SHIMADZU (HPLC-01)): Column, Kinetex EVO prep C18, 30*150, 5 um; mobile phase, Water (10 mmol/L NH₄HCO₃) and ACN (20% ACN up to 50% in 8 min) to afford 2-{6-[3-(cyclobutylamino)pyrrolidin-1-yl]pyridazin-3-yl}-5-(2-methyl-1,3-oxazol-5-yl)phenol (8 mg, 22%) as a solid. LCMS (ES, m/z): 391.8 [M+H]^{+ 1}H NMR (400 MHz, DMSO-d₆) δ 14.03 (s, 1H), 8.22 (d, J=9.9 Hz, 1H), 7.94 (d, J=8.9 Hz, 1H), 7.60 (s, 1H), 7.24-7.19 (m, 2H), 7.14 (d, J=9.8 Hz, 1H), 3.63 (dt, J=15.4, 7.7 Hz, 2H), 3.56-3.43 (m, 1H), 3.38 (t, J=5.7 Hz, 1H), 3.29-3.19 (m, 2H), 2.49 (s, 3H), 2.23-2.01 (m, 4H), 1.88-1.76 (m, 1H), 1.76-1.63 (m, 2H), 1.59 (dd, J=11.7, 9.3 Hz, 2H).

### Example 103: Synthesis of Compound 348

### Synthesis of Intermediate B262

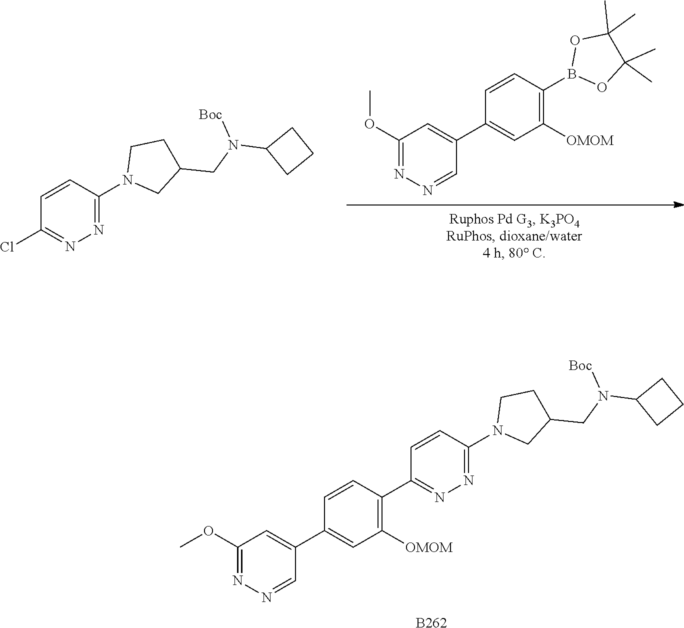

To a solution of tert-butyl N-{[1-(6-chloropyridazin-3-yl)pyrrolidin-3-yl]methyl}-N-cyclobutylcarbamate (69.0 mg, 0.188 mmol, 0.7 equiv) and 3-methoxy-5-[3-(methoxymethoxy)-4-(4,4,5,5-tetramethyl-1,3,2-dioxaborolan-2-yl)phenyl]pyridazine (100 mg, 0.269 mmol, 1.00 equiv) in dioxane (1 mL) and water (0.2 mL) were added K₃PO₄ (171.08 mg, 0.807 mmol, 3 equiv) and RuPhos Palladacycle Gen.3 (11.23 mg, 0.013 mmol, 0.05 equiv) and RuPhos (12.54 mg, 0.027 mmol, 0.1 equiv). After stirring for 4 h at 80° C. under a nitrogen atmosphere. The mixture was allowed to cool down to room temperature. The reaction was quenched by the addition of Water (5 mL) at room temperature. The aqueous layer was extracted with EtOAc (3×10 mL). The resulting mixture was concentrated under reduced pressure. The residue was dissolved in CH₂Cl₂ (5 mL). The residue was purified by silica gel column chromatography, eluted with CH₂Cl₂/MeOH (5:1) to afford tert-butyl N-cyclobutyl-N-[(1-{6-[2-(methoxymethoxy)-4-(6-methoxypyridazin-4-yl)phenyl]pyridazin-3-yl}pyrrolidin-3-yl)methyl]carbamate (120 mg, 77%) as an oil. LCMS:(ES, m/z):577.20[M+H]

### Synthesis of Compound 348

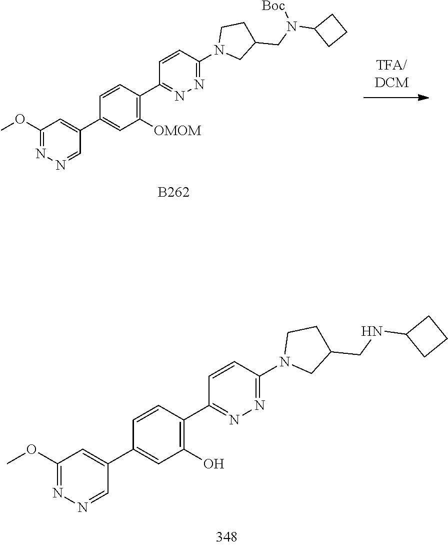

To a stirred mixture of tert-butyl N-cyclobutyl-N-[(1-{6-[2-(methoxymethoxy)-4-(6-methoxypyridazin-4-yl)phenyl]pyridazin-3-yl}pyrrolidin-3-yl)methyl]carbamate (120 mg, 0.208 mmol, 1 equiv) in DCM (2 mL) was added TFA (1 mL) dropwise at 0° C. under air atmosphere.

The reaction was quenched by the addition of MeOH (10 mL) at 0° C. The resulting mixture was concentrated under reduced pressure. The mixture was basified to pH 8 with NH₃·H₂O. The resulting mixture was extracted with EtOAc (2×30 mL), dried over anhydrous Na₂SO₄. After filtration, the filtrate was concentrated under reduced pressure. The crude product (50 mg) was purified by Prep-HPLC with the following conditions (Column, Kinetex EVO prep C18, 30×150, 5 um; mobile phase, Water (10 mmol/L NH₄HCO₃) and ACN (15% ACN up to 46% in 10 min)) to afford 2-(6-{3-[(cyclobutylamino)methyl]pyrrolidin-1-yl}pyridazin-3-yl)-5-(6-methoxypyridazin-4-yl)phenol (60.1 mg, 67%) as a solid. LCMS:(ES, m/z):432.95[M+H1H NMR:(ES, m/z):(400 MHz, DMSO-d₆) δ 14.11 (s, 1H), 9.34 (d, J=1.9 Hz, 1H), 8.30 (dd, J=10.0, 1.8 Hz, 1H), 8.04 (d, J=8.1 Hz, 1H), 7.57-7.52 (m, 1H), 7.52-7.43 (m, 2H), 7.21-7.14 (m, 1H), 4.09 (s, 3H), 3.69 (t, J=9.5 Hz, 2H), 3.63 (s, 1H), 3.49 (q, J=8.3 Hz, 2H), 3.25-3.14 (m, 1H), 2.56 (dd, J=11.3, 6.5 Hz, 1H), 2.49-2.37 (m, 1H), 2.16-2.05 (m, 3H), 1.68 (s, 3H), 1.68-1.50 (m, 2H).

### Example 104: Synthesis of Compound 349

### Synthesis of Intermediate B263

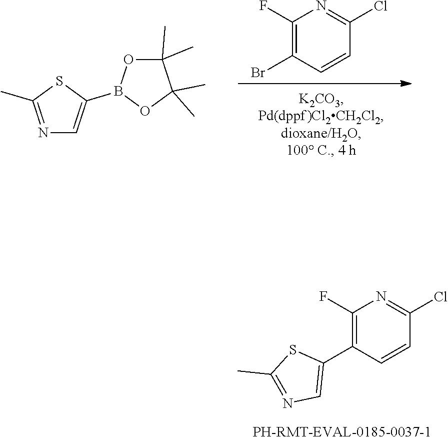

A solution of 2-methyl-5-(4,4,5,5-tetramethyl-1,3,2-dioxaborolan-2-yl)-1,3-thiazole (1 g, 4.442 mmol, 1 equiv), 3-bromo-6-chloro-2-fluoropyridine (1.12 g, 5.330 mmol, 1.2 equiv), K₂CO₃ (1.23 g, 8.884 mmol, 2 equiv), Pd(dppf)Cl₂·CH₂Cl₂ (0.18 g, 0.222 mmol, 0.05 equiv) in dioxane and water was stirred at 100° C. for 4 h under N₂ balloon. LCMS showed the obtaining of the product and byproduct (over reaction). The mixture was dried over with Na₂SO₄ and the crude was purified by column chromatograph on silica gel (10˜30% EA in PE) to give product LCMS:(ES, m/z): 229 [M+H]⁺

### Synthesis of Intermediate B264

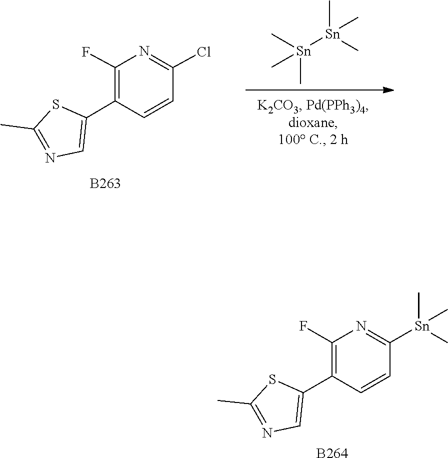

A solution of 6-chloro-2-fluoro-3-(2-methyl-1,3-thiazol-5-yl)pyridine (200 mg, 0.875 mmol, 1 equiv), hexamethyldistannane (573.11 mg, 1.750 mmol, 2 equiv), Pd(PPh₃)₄(50.54 mg, 0.044 mmol, 0.05 equiv) in dioxane was stirred at 100° C. for 2 h. The mixture was quenched by KF solution and extracted with EtOAc. The organic layer was washed with water and brine, dried over with Na₂SO4 and concentrated. The crude was used to the next step directly. LCMS:(ES, m/z): 359 [M+H]⁺

### Synthesis of Intermediate B265

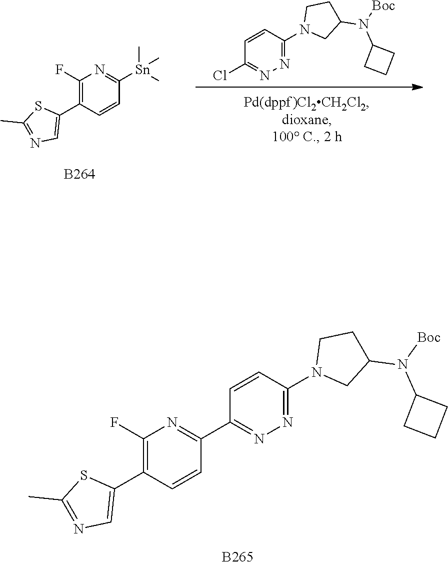

A solution of 2-fluoro-3-(2-methyl-1,3-thiazol-5-yl)-6-(trimethylstannyl)pyridine (300 mg, 0.840 mmol, 1 equiv), tert-butyl N-[1-(6-chloropyridazin-3-yl)pyrrolidin-3-yl]-N-cyclobutylcarbamate (148.25 mg, 0.420 mmol, 0.5 equiv), Pd(dppf)Cl₂·CH₂Cl₂ (34.22 mg, 0.042 mmol, 0.05 equiv) in dioxane was stirred at 100° C. for 2h. LCMS showed the obtaining of the product. The mixture was concentrated and the crude was purified by column chromatograph on silica gel (PE:EA=5:1-EA) to give product. LCMS:(ES, m/z): 511 [M+H]⁺

### Synthesis of Compound 349

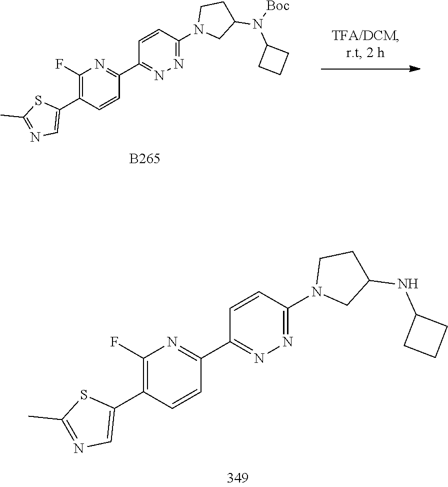 [SMILES: Cc1ncc(-c2ccc(-c3ccc(N4CCC(N(C(=O)OC(C)(C)C)C5CCC5)C4)nn3)nc2F)s1 ; "B265" ; Cc1ncc(-c2ccc(-c3ccc(N4CCC(NC5CCC5)C4)nn3)nc2F)s1]

To a solution of tert-butyl N-cyclobutyl-N-(1-{6-[6-fluoro-5-(2-methyl-1,3-thiazol-5-yl)pyridin-2-yl]pyridazin-3-yl}pyrrolidin-3-yl)carbamate (300 mg, 0.235 mmol, 1 equiv, 40%) in DCM (5 mL) was added TFA (1 mL) and the mixture was stirred at room temperature for 2h.

The mixture was concentrated and the residue was extracted with EtOAc for three times. The aqueous layer was basified by NH₃·H₂O and extracted with DCM. The organic layer was dried over with Na₂SO4 and concentrated. The crude product was purified by Chiral-Prep-HPLC with the following conditions (2 #SHIMADZU (HPLC-01)): Column, XBridge Shield RP18 OBD Column, 30*150 mm, 5 μm; mobile phase, Water (10 mmol/L NH₄HCO₃) and ACN (20% ACN up to 58% in 8 min); Detector, UV to give product as white solid. LCMS:(ES, m/z): 411 [M+H]+H-NMR: (400 MHz, DMSO-d₆) δ 8.44 (dd, J=10.0, 8.0 Hz, 1H), 8.35 (dd, J=8.0, 2.1 Hz, 1H), 8.23 (s, 1H), 8.07 (d, J=9.5 Hz, 1H), 6.97 (d, J=9.6 Hz, 1H), 3.65 (s, 2H), 3.51 (s, 1H), 3.40 (p, J=5.7 Hz, 1H), 3.25 (q, J=7.7 Hz, 2H), 2.73 (s, 3H), 2.22-2.04 (m, 3H), 1.83 (dq, J 13.0, 6.7 Hz, 1H), 1.69 (dd, J=9.5, 3.3 Hz, 2H), 1.64-1.50 (m, 2H). - 2.06 (m, 4H), 1.83 (dt, J=12.4, 6.6 Hz, 1H), 1.76-1.63 (m, 2H), 1.63-1.47 (m, 3H).

### Example 105: Synthesis of Compound 350

### Synthesis of Intermediate B266

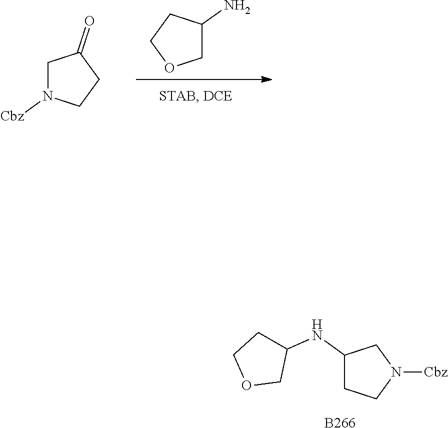

A solution of benzyl 3-oxopyrrolidine-1-carboxylate (1 g, 4.561 mmol, 1 equiv) and oxolan-3-amine (0.40 g, 4.561 mmol, 1 equiv) and STAB (2.90 g, 13.683 mmol, 3 equiv) in DCE (20 mL) was stirred for 4 h at 25° C. The reaction was quenched by the addition of Water (5 mL) at room temperature. The aqueous layer was extracted with EtOAc (3×10 mL). The resulting mixture was concentrated under reduced pressure. The residue was dissolved in CH₂Cl₂ (5 mL).

The residue was purified by silica gel column chromatography, eluted with CH₂Cl₂/MeOH (5:1) to afford benzyl 3-(oxolan-3-ylamino)pyrrolidine-1-carboxylate (1 g, 76%) as a oil.

LCMS:(ES, m/z):290.90[M+H]⁺

### Synthesis of Intermediate B267

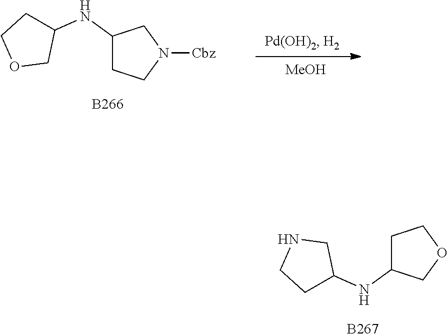

A solution of benzyl 3-(oxolan-3-ylamino)pyrrolidine-1-carboxylate (1 g, 3.444 mmol, 1 equiv) and Pd(OH)₂/C (300 mg, 2.136 mmol, 0.62 equiv) in MeOH(20 mL) was stirred for 16 h at 25° C. under hydrogen atmosphere. The crude product was used in the next step directly without further purification. LCMS:(ES, m/z):156.95[M+H]⁺

### Synthesis of Intermediate B268

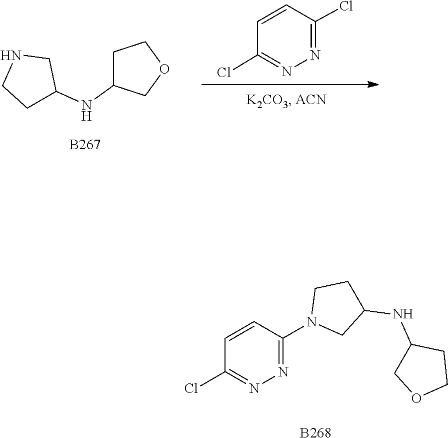

A solution of N-(oxolan-3-yl)pyrrolidin-3-amine (1 g, 6.401 mmol, 1 equiv) in ACN (10 mL) was treated with K₂CO₃ (4.42 g, 32.005 mmol, 5 equiv) for 4 h at 80° C. under nitrogen atmosphere followed by the addition of pyridazine, 3,6-dichloro- (0.95 g, 6.401 mmol, 1 equiv).

The aqueous layer was extracted with CH₂Cl₂ (2×200 mL). The residue was purified by silica gel column chromatography, eluted with CH₂Cl₂/MeOH (5:1) to afford tert-butyl N-[1-(6-chloropyridazin-3-yl)pyrrolidin-3-yl]-N-(oxolan-3-yl)carbamate (280 mg, 12%) as a oil.

LCMS:(ES, m/z):268.95[M+H]⁺

### Synthesis of Intermediate B269

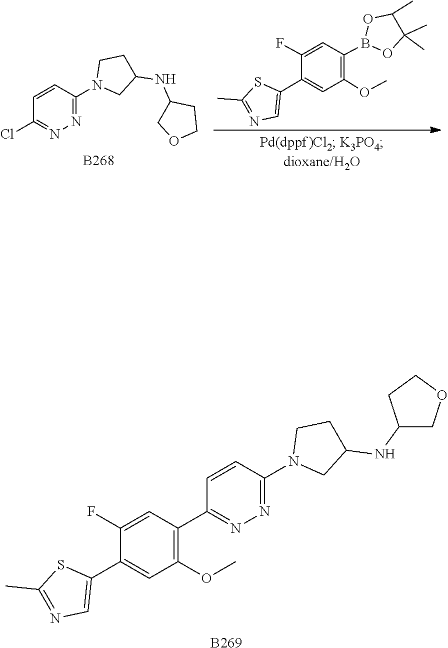

To a solution of 1-(6-chloropyridazin-3-yl)-N-(oxolan-3-yl)pyrrolidin-3-amine (61.56 mg, 0.229 mmol, 0.8 equiv) and 5-[2-fluoro-5-methoxy-4-(4,4,5,5-tetramethyl-1,3,2-dioxaborolan-2-yl)phenyl]-2-methyl-1,3-thiazole (100 mg, 0.286 mmol, 1.00 equiv) in dioxane (2 mL) and water (0.4 mL) were added K₃PO₄ (182.34 mg, 0.858 mmol, 3 equiv) and Pd(dppf)Cl₂·CH₂Cl₂ (23.33 mg, 0.029 mmol, 0.1 equiv). After stirring for 4 h at 80° C. under a nitrogen atmosphere. The mixture was allowed to cool down to room temperature, the resulting mixture was concentrated under reduced pressure. The reaction was quenched by the addition of Water (5 mL) at room temperature. The aqueous layer was extracted with EtOAc (3×10 mL). The resulting mixture was concentrated under reduced pressure. The residue was dissolved in CH₂Cl₂ (5 mL). The residue was purified by Prep-TLC gel column chromatography, eluted with CH₂Cl₂/MeOH (5:1) to afford 1-{6-[5-fluoro-2-methoxy-4-(2-methyl-1,3-thiazol-5-yl)phenyl]pyridazin-3-yl}-N-(oxolan-3-yl)pyrrolidin-3-amine as a solid. LCMS:(ES, m/z):455.85[M+H]⁺

### Synthesis of Compound 350

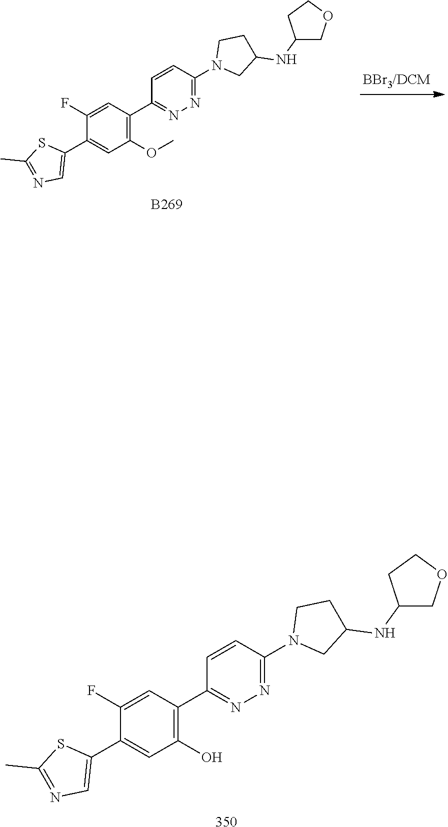 [SMILES: COc1cc(-c2cnc(C)s2)c(F)cc1-c1ccc(N2CCC(NC3CCOC3)C2)nn1 ; "B269" ; Cc1ncc(-c2cc(O)c(-c3ccc(N4CCC(NC5CCOC5)C4)nn3)cc2F)s1 ; "350"]

To a stirred mixture of 1-{6-[5-fluoro-2-methoxy-4-(2-methyl-1,3-thiazol-5-yl)phenyl]pyridazin-3-yl}-N-(oxolan-3-yl)pyrrolidin-3-amine (60 mg, 0.132 mmol, 1 equiv) in DCM (1 mL) was added TFA (0.5 mL) dropwise at 0° C. under air atmosphere. The reaction was quenched by the addition of MeOH (5 mL) at 0° C. The resulting mixture was concentrated under reduced pressure. The mixture was basified to pH 8 with NH₃-H₂O. The resulting mixture was extracted with EtOAc (2×30 mL), dried over anhydrous Na₂SO₄. After filtration, the filtrate was concentrated under reduced pressure. The crude product (50 mg) was purified by Prep-HPLC with the following conditions (Column, Kinetex EVO prep C18, 30×150, 5 um; mobile phase, Water (10 mmol/L NH₄HCO₃) and ACN (10% ACN up to 51% in 8 min)) to afford 4-fluoro-5-(2-methyl-1,3-thiazol-5-yl)-2-{6-[3-(oxolan-3-ylamino)pyrrolidin-1-yl]pyridazin-3-yl}phenol (9.5 mg, 16%) as a solid. LCMS:(ES, m/z):441.80[M+H]^{+ 1}HNMR:(400 MHz, DMSO-d₆) δ 13.72 (s, 1H), 8.25 (d, J=9.6 Hz, 1H), 8.16 (s, 1H), 7.93 (dd, J=12.7, 2.6 Hz, 1H), 7.29 (d, J=6.8 Hz, 1H), 7.15 (t, J=9.8 Hz, 1H), 4.53 (d, J=16.5 Hz, 1H), 3.58 (s, 4H), 3.28 (s, 1H), 3.14 (s, 2H), 2.93-2.84 (m, 2H), 2.71 (s, 3H), 2.06 (s, 3H), 1.76 (d, J=8.7 Hz, 1H). ¹⁹F NMR: (376 MHz, DMSO-d6) δ−125.47.

### Example 106: Synthesis of Compound 351

### Synthesis of Intermediate B270

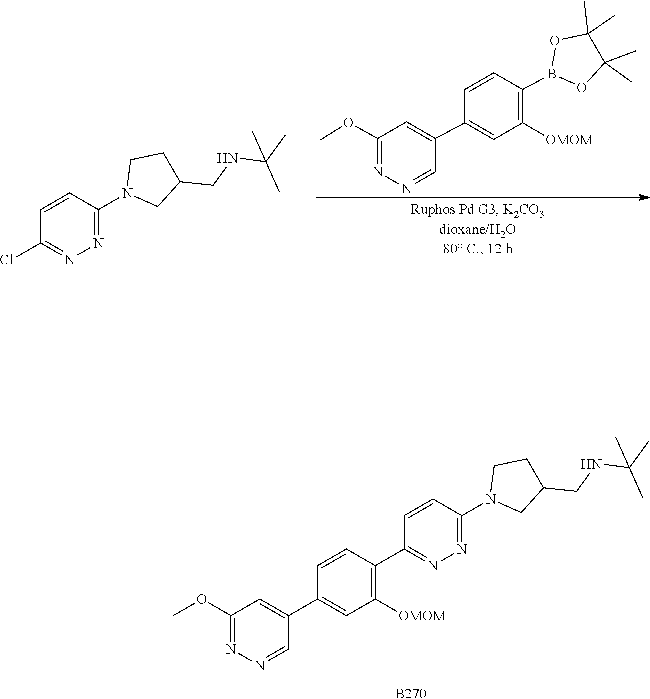

A mixture of tert-butyl({[1-(6-chloropyridazin-3-yl)pyrrolidin-3-yl]methyl})amine (400 mg, 1.488 mmol, 1 equiv) and 3-methoxy-5-[3-(methoxymethoxy)-4-(4,4,5,5-tetramethyl-1,3,2-dioxaborolan-2-yl)phenyl]pyridazine (664.72 mg, 1.786 mmol, 1.2 equiv) and RuPhos Palladacycle Gen.3 (124.47 mg, 0.149 mmol, 0.1 equiv) and K₂CO₃ (411.34 mg, 2.976 mmol, 2 equiv) in dioxane (6 mL) and H₂O (1.2 mL) was stirred for 12 h at 80° C. under nitrogen atmosphere. The resulting mixture was concentrated under reduced pressure. The residue was purified by silica gel column chromatography, eluted with CH₂Cl₂/MeOH (10:1) to afford tert-butyl[(1-{6-[2-(methoxymethoxy)-4-(6-methoxypyridazin-4-yl)phenyl]pyridazin-3-yl}pyrrolidin-3-yl)methyl]amine amine (190 mg, 26%).

### Synthesis of Compound 351

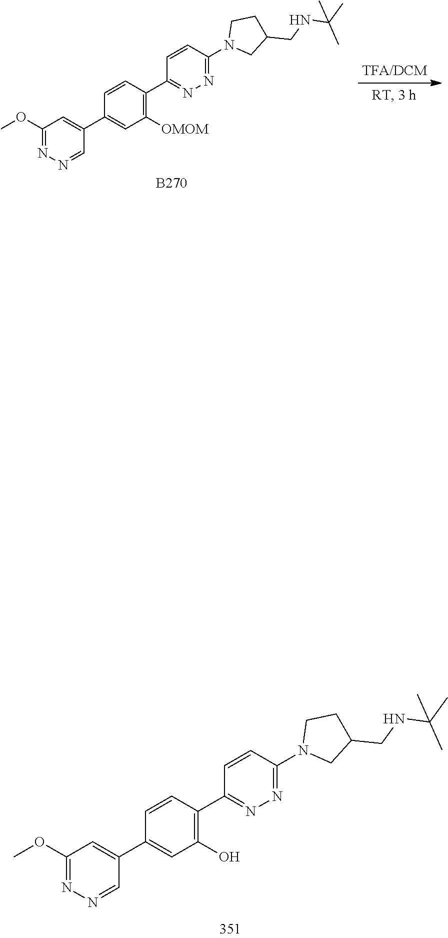

A solution of tert-butyl[(1-{6-[2-(methoxymethoxy)-4-(6-methoxypyridazin-4-yl)phenyl]pyridazin-3-yl}pyrrolidin-3-yl)methyl]amine (190 mg, 0.397 mmol, 1 equiv) and TFA (1.5 mL) in DCM (4.5 mL) was stirred for 3 h at room temperature under air atmosphere. The resulting mixture was concentrated under reduced pressure. The residue was neutralized to pH 10 with NH₃·H₂O. The aqueous layer was extracted with CH₂Cl₂ (3×10 mL). The crude product was purified by Chiral-Prep-HPLC with the following conditions (SHIMADZU (HPLC-01): Column, Kinetex EVO prep C18, 30*150, 5 um; mobile phase, Water (10 mmol/L NH₄HCO₃) and ACN (5% ACN up to 45% in 10 min); Detector, UV 220 nm) to afford 2-(6-{3-[(tert-butylamino)methyl]pyrrolidin-1-yl}pyridazin-3-yl)-5-(6-methoxypyridazin-4-yl)phenol (14 mg, 8%) as a solid. LCMS:(ES, m/z): 435 [M+H]⁺¹HNMR: (400 MHz, DMSO-d₆) δ 14.07 (s, 1H), 9.35 (d, J=1.9 Hz, 1H), 9.34-8.34 (m, 2H), 8.05 (d, J=8.0 Hz, 1H), 7.55 (s, 1H), 7.49 (s, 2H), 7.19 (d, J=9.8, 1H), 4.09 (s, 3H), 3.78 (s, 1H), 3.66 (s, 1H), 3.52 (d, J=9.5 Hz, 1H), 3.41-3.39 (m, 1H), 3.31-3.27 (m, 2H), 2.84-2.66 (m, 1H), 2.32-2.19 (m, 1H), 1.98-1.81 (m, 1H), 1.18 (s, 9H).

### Example 107: Synthesis of Compound 352

### Synthesis of Intermediate B271

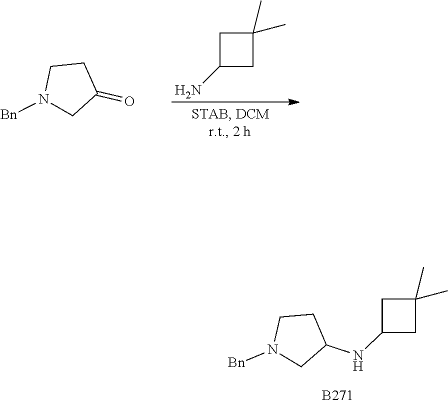

To a solution of 1-benzylpyrrolidin-3-one (1 g, 5.707 mmol, 1 equiv) in DCM (10 mL) was added 3,3-dimethylcyclobutan-1-amine (0.57 g, 5.707 mmol, 1 equiv) and STAB (2.42 g, 11.414 mmol, 2 equiv) at 0° C. After stirring for 3 h at 25° C. under a nitrogen atmosphere. The reaction was quenched by the addition of ice water(10 mL) at 0° C. The mixture was basified to pH 10 with Na₂CO₃. The resulting mixture was extracted with CH₂Cl₂ (3×10 mL). The combined organic layers were washed with brine (1×10 mL), dried over anhydrous Na₂SO₄. After filtration, the filtrate was concentrated under reduced pressure to afford crude 1-benzyl-N-(3,3-dimethylcyclobutyl)pyrrolidin-3-amine (1 g, 68%) as an oil.

### Synthesis of Intermediate B272

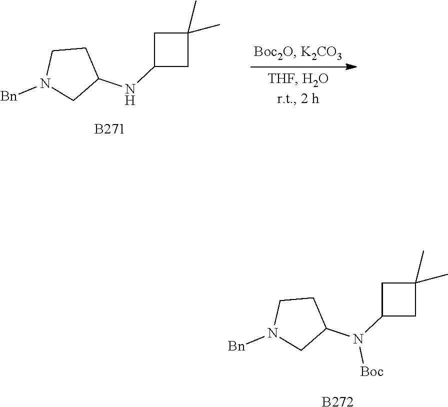

To a solution of 1-benzyl-N-(3,3-dimethylcyclobutyl)pyrrolidin-3-amine (1 g, 3.870 mmol, 1 equiv) in THF (10 mL) were added K₂CO₃ (1.07 g, 7.742 mmol, 2 equiv) in H₂O (2 mL) and Boc₂O (1.27 g, 5.805 mmol, 1.5 equiv) was added to the reaction. After stirring for 2 h at 25° C. under a nitrogen atmosphere. The resulting mixture was extracted with EtOAc (3×30 mL). The combined organic layers were washed with brine (1×10 mL), dried over anhydrous Na₂SO₄.

After filtration, the filtrate was concentrated under reduced pressure. the resulting mixture was concentrated under reduced pressure. The residue was purified by Prep-TLC/silica gel column chromatography, eluted with PE/EA (3:1) to afford tert-butyl (1-benzylpyrrolidin-3-yl)(3,3-dimethylcyclobutyl)carbamate (1 g, 72.08%) as a brown oil.

### Synthesis of Intermediate B273

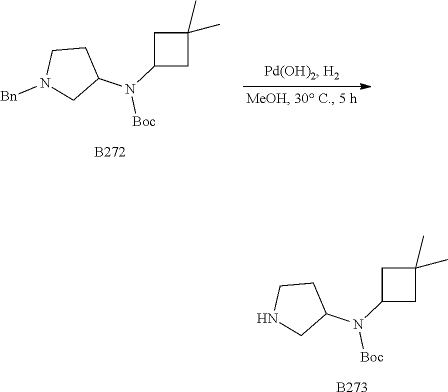

To a solution of tert-butyl (1-benzylpyrrolidin-3-yl)(3,3-dimethylcyclobutyl)carbamate (1 g, 2.789 mmol, 1 equiv) in MeOH (10 mL) was added Pd(OH)2/C (0.3 g) under nitrogen atmosphere in a 100 mL round-bottom flask. The mixture was hydrogenated at 30° C. for 5 h under hydrogen atmosphere using a hydrogen balloon. The resulting mixture was filtered and the filter cake was washed with MeOH (30×10 mL). The filtrate was concentrated under reduced pressure to afford tert-butyl (3,3-dimethylcyclobutyl)(pyrrolidin-3-yl)carbamate (600 mg, 80.15%) as an oil.

### Synthesis of Intermediate B274

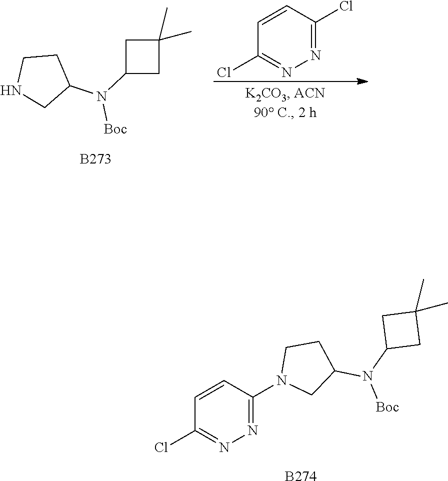

To a solution of tert-butyl (3,3-dimethylcyclobutyl)(pyrrolidin-3-yl)carbamate (300 mg, 1.118 mmol, 1 equiv) and pyridazine, 3,6-dichloro- (166.51 mg, 1.118 mmol, 1 equiv) in ACN (5 mL) were added K₂CO₃ (463.43 mg, 3.354 mmol, 3 equiv). After stirring for 2 h at 90° C. under a nitrogen atmosphere. The mixture was allowed to cool down to 25° C. The resulting mixture was filtered and the filter cake was washed with DCM(2×10 mL). The filtrate was concentrated under reduced pressure. The residue was purified by Prep-TLC/silica gel column chromatography, eluted with CH₂Cl₂/ PE (1:1) to afford tert-butyl (1-(6-chloropyridazin-3-yl)pyrrolidin-3-yl)(3,3-dimethylcyclobutyl)carbamate (300 mg, 71%) as a solid.

### Synthesis of Intermediate B275

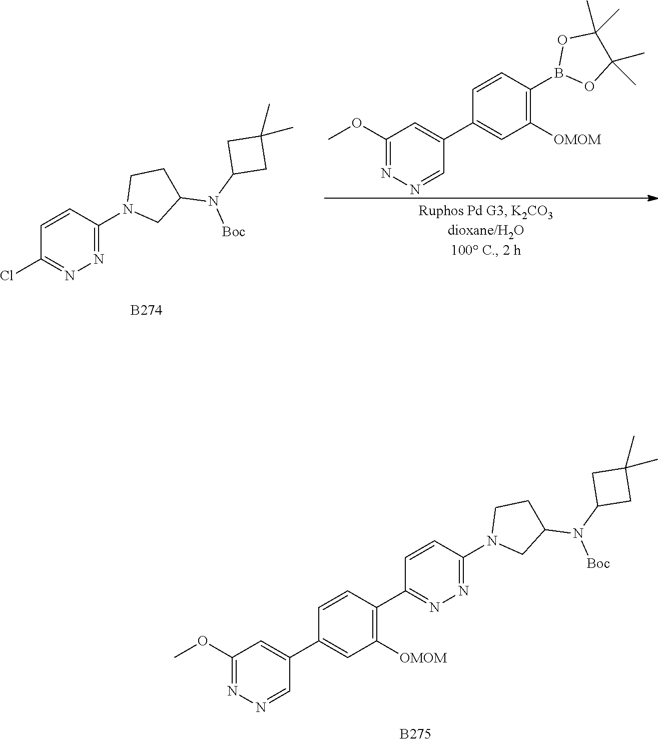

To a solution of tert-butyl (1-(6-chloropyridazin-3-yl)pyrrolidin-3-yl)(3,3-dimethylcyclobutyl)carbamate (100 mg, 0.263 mmol, 1 equiv) and 3-methoxy-5-[3-(methoxymethoxy)-4-(4,4,5,5-tetramethyl-1,3,2-dioxaborolan-2-yl)phenyl]pyridazine (107.49 mg, 0.289 mmol, 1.1 equiv) in 1,4-dioxane (2 mL) and H₂O (0.4 mL) were added 3rd Generation RuPhos precatalyst (21.96 mg, 0.026 mmol, 0.1 equiv) and K₂CO₃ (108.85 mg, 0.789 mmol, 3 equiv). After stirring for 2 h at 100° C. under a nitrogen atmosphere, the resulting mixture was concentrated under reduced pressure. The residue was purified by Prep-TLC/silica gel column chromatography, eluted with PE/EA (1/7) to afford tert-butyl (3,3-dimethylcyclobutyl)(1-(6-(2-(methoxymethoxy)-4-(6-methoxypyridazin-4-yl)phenyl)pyridazin-3-yl)pyrrolidin-3-yl)carbamate (80 mg, 52%) as a solid.

### Synthesis of Compound 352

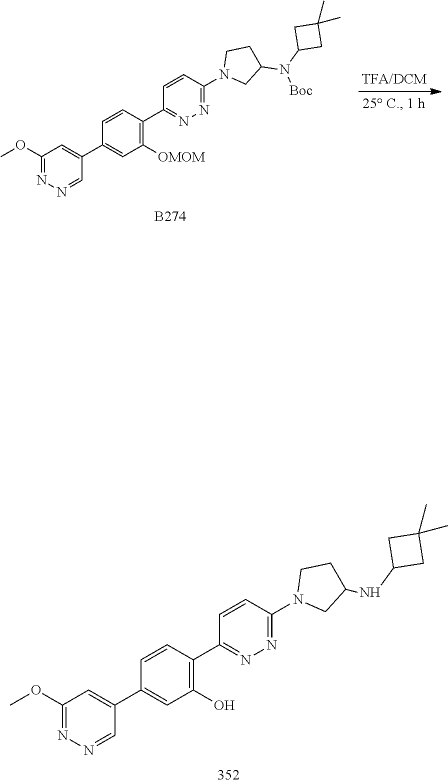

To a solution of tert-butyl N-(3,3-dimethylcyclobutyl)-N-(1-{6-[2-(methoxymethoxy)-4-(6-methoxypyridazin-4-yl)phenyl]pyridazin-3-yl}pyrrolidin-3-yl)carbamate (80 mg, 0.135 mmol, 1 equiv) in DCM (2 mL) were added TFA (0.5 mL). After stirring for 1 h at 25° C. under a nitrogen atmosphere, the resulting mixture was concentrated under reduced pressure. The crude product was purified by Chiral-Prep-HPLC with the following conditions (2 #SHIMADZU (HPLC-01)): Column, Kinetex EVO prep C18, 30*150, 5 um; mobile phase, Water (10 mmol/L NH₄HCO₃) and ACN (25% ACN up to 61% in 8 min) to afford 2-(6-{3-[(3,3-dimethylcyclobutyl)amino]pyrrolidin-1-yl}pyridazin-3-yl)-5-(6-methoxypyridazin-4-yl)phenol (16.2 mg, 26.79%) as a solid. LCMS:(ES, m/z):446.9[M+H]¹HNMR (400 MHz, DMSO-d₆) δ 14.11 (s, 1H), 9.35 (d, J=1.9 Hz, 1H), 8.29 (d, J=9.8 Hz, 1H), 8.04 (d, J=8.2 Hz, 1H), 7.55 (d, J=1.9 Hz, 2H), 7.53-7.43 (m, 2H), 7.17 (d, J=9.8 Hz, 1H), 4.09 (s, 3H), 3.63 (dd, J=11.0, 5.6 Hz, 2H), 3.51 (s, 1H), 3.36 (t, J=5.5 Hz, 1H), 3.26 (dd, J=17.3, 9.6 Hz, 2H), 2.09 (dt, J=12.6, 6.7 Hz, 1H), 1.98 (dd, J=6.9, 4.4 Hz, 2H), 1.83 (dd, J=12.5, 6.4 Hz, 1H), 1.52 (ddd, J=11.9, 8.2, 4.2 Hz, 2H), 1.08 (d, J=14.7 Hz, 6H).

### Example 108: Synthesis of Compound 353

### Synthesis of Intermediate B275

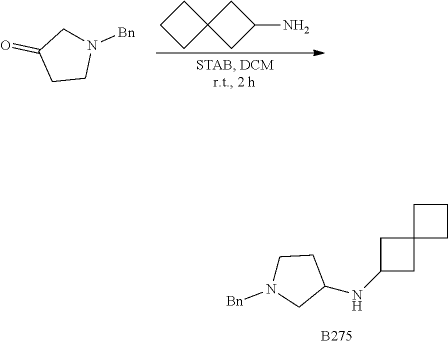

To a solution of spiro[3.3]heptan-2-amine (1 g, 5.707 mmol, 1 equiv) in DCM (10 mL) was added 3,3-dimethylcyclobutan-1-amine (0.63 g, 5.707 mmol, 1 equiv) and STAB (2.42 g, 11.414 mmol, 2 equiv) at 0° C. After stirring for 3 h at 25° C. under a nitrogen atmosphere. The reaction was quenched by the addition of ice water(10 mL) at 0° C. The mixture was basified to pH 10 with Na₂CO₃. The resulting mixture was extracted with CH₂Cl₂ (3×10 mL). The combined organic layers were washed with brine (1×10 mL), dried over anhydrous Na₂SO₄. After filtration, the filtrate was concentrated under reduced pressure to afford crude 1-benzyl-N-(spiro[3.3]heptan-2-yl)pyrrolidin-3-amine (1 g, 64.8%) as a brown oil.

### Synthesis of Intermediate B276

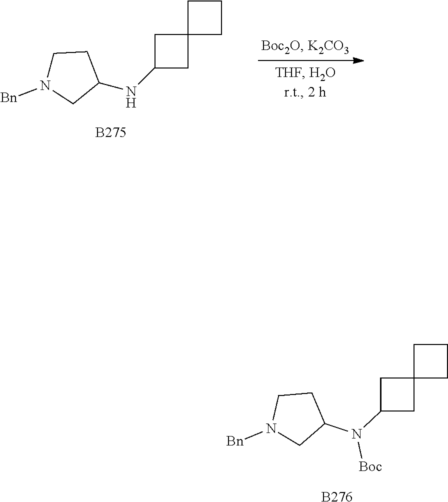

To a solution of 1-benzyl-N-(spiro[3.3]heptan-2-yl)pyrrolidin-3-amine (1 g, 3.698 mmol, 1 equiv) in THF (10 mL) were added K₂CO₃ (1.02 g, 7.396 mmol, 2 equiv) in H₂O (2 mL) and Boc₂O (1.21 g, 5.547 mmol, 1.5 equiv) was added to the reaction. After stirring for 2 h at 25° C. under a nitrogen atmosphere. The resulting mixture was extracted with EtOAc (3×30 mL). The combined organic layers were washed with brine (1×10 mL), dried over anhydrous Na₂SO₄.

After filtration, the filtrate was concentrated under reduced pressure. the resulting mixture was concentrated under reduced pressure. The residue was purified by Prep-TLC/silica gel column chromatography, eluted with PE/EA (3:1) to afford tert-butyl (1-benzylpyrrolidin-3-yl)(spiro[3.3]heptan-2-yl)carbamate (1 g, 73%) as an oil.

### Synthesis of Intermediate B277

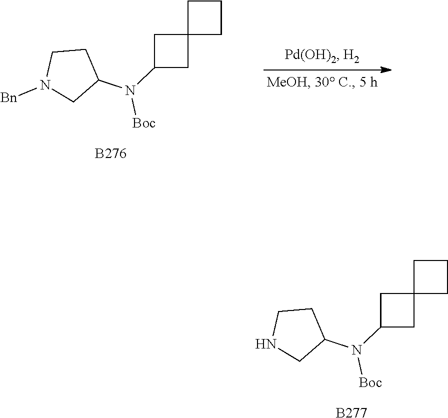

To a solution of tert-butyl (1-benzylpyrrolidin-3-yl)(spiro[3.3]heptan-2-yl)carbamate (1 g, 2.699 mmol, 1 equiv) in MeOH (10 mL) was added Pd(OH)₂/C (0.3 g) under nitrogen atmosphere in a 100 mL round-bottom flask. The mixture was hydrogenated at 30° C. for 5 h under hydrogen atmosphere using a hydrogen balloon. The resulting mixture was filtered and the filter cake was washed with MeOH (30×10 mL). The filtrate was concentrated under reduced pressure to afford tert-butyl pyrrolidin-3-yl(spiro[3.3]heptan-2-yl)carbamate (600 mg, 79.28%) as an oil.

### Synthesis of Intermediate B278

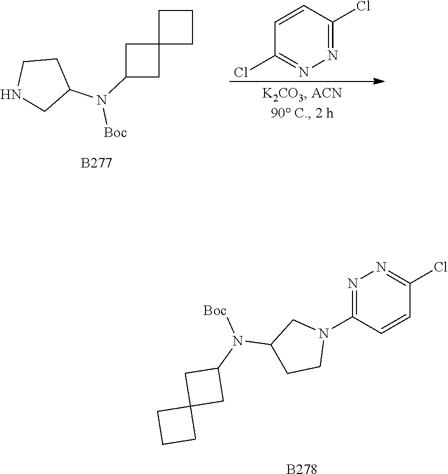

To a solution of tert-butyl pyrrolidin-3-yl(spiro[3.3]heptan-2-yl)carbamate (300 mg, 1.070 mmol, 1 equiv) and pyridazine, 3,6-dichloro- (159.38 mg, 1.070 mmol, 1 equiv) in ACN (5 mL) were added K₂CO₃ (443.58 mg, 3.210 mmol, 3 equiv). After stirring for 2 h at 90° C. under a nitrogen atmosphere. The mixture was allowed to cool down to 25° C. The resulting mixture was filtered and the filter cake was washed with DCM(2×10 mL). The filtrate was concentrated under reduced pressure. The residue was purified by Prep-TLC/silica gel column chromatography, eluted with EA/PE (1:1) to afford tert-butyl (1-(6-chloropyridazin-3-yl)pyrrolidin-3-yl)(spiro[3.3]heptan-2-yl)carbamate (300 mg, 71%) as a solid.

### Synthesis of Intermediate B279

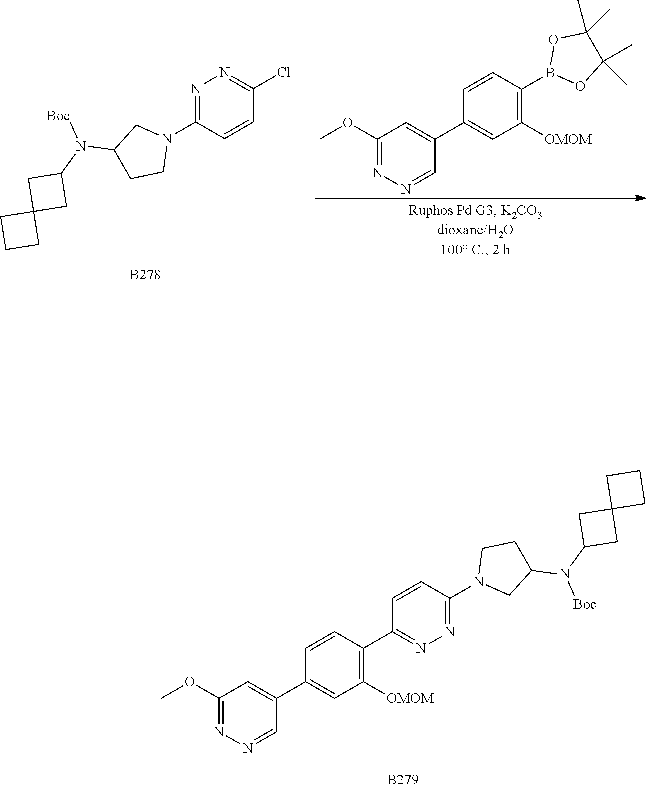

To a solution of tert-butyl (1-(6-chloropyridazin-3-yl)pyrrolidin-3-yl)(spiro[3.3]heptan-2-yl)carbamate (100 mg, 0.254 mmol, 1 equiv) and 3-methoxy-5-[3-(methoxymethoxy)-4-(4,4,5,5-tetramethyl-1,3,2-dioxaborolan-2-yl)phenyl]pyridazine (104.21 mg, 0.279 mmol, 1.1 equiv) in 1,4-dioxane (2 mL) and H₂O (0.4 mL) were added 3rd Generation RuPhos precatalyst (15.88 mg, 0.025 mmol, 0.1 equiv) and K₂CO₃ (105.52 mg, 0.762 mmol, 3 equiv). After stirring for 2 h at 100° C. under a nitrogen atmosphere, the resulting mixture was concentrated under reduced pressure. The residue was purified by Prep-TLC/silica gel column chromatography, eluted with PE/EA (1/7) to afford tert-butyl (1-(6-(2-(methoxymethoxy)-4-(6-methoxypyridazin-4-yl)phenyl)pyridazin-3-yl)pyrrolidin-3-yl)(spiro[3.3]heptan-2-yl)carbamate (80 mg, 52%) as a solid.

### Synthesis of Compound 353

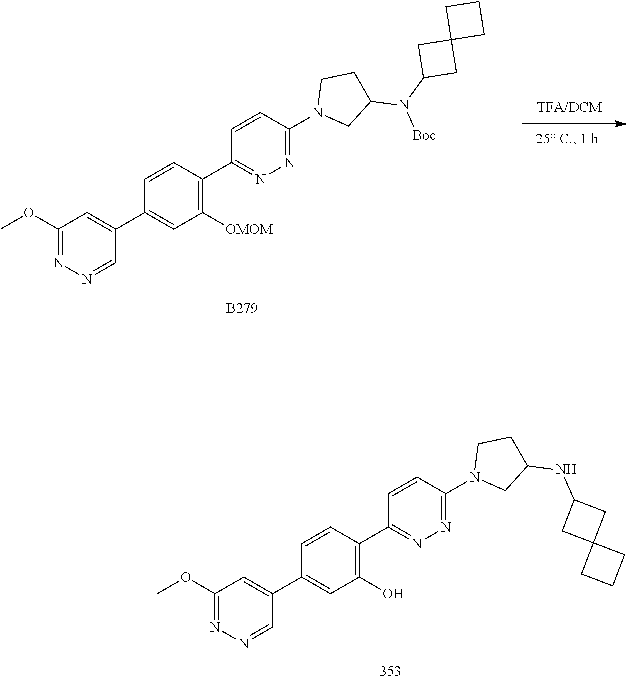

To a solution of tert-butyl N-(1-{6-[2-(methoxymethoxy)-4-(6-methoxypyridazin-4-yl)phenyl]pyridazin-3-yl}pyrrolidin-3-yl)-N-{spiro[3.3]heptan-2-yl}carbamate (80 mg, 0.133 mmol, 1 equiv) in DCM (1 mL, 15.731 mmol, 118.52 equiv) were added TFA (0.5 mL, 6.732 mmol, 50.72 equiv). After stirring for 1 h at 25° C. under a nitrogen atmosphere, the resulting mixture was concentrated under reduced pressure. The crude product was purified by Chiral-Prep-HPLC with the following conditions (2 #SHIMADZU (HPLC-01)): Column, Kinetex EVO prep C18, 30*150, 5 um; mobile phase, Water (10 mmol/L NH₄HCO₃) and ACN (20% ACN up to 65% in 8 min) to afford 5-(6-methoxypyridazin-4-yl)-2-[6-(3-{spiro[3.3]heptan-2-ylamino}pyrrolidin-1-yl)pyridazin-3-yl]phenol (12.9 mg, 21%) as a solid. LCMS:(ES, m/z):458.9 ¹HNMR: (400 MHz, DMSO-d₆) δ 14.11 (s, 1H), 9.35 (d, J=1.8 Hz, 1H), 8.29 (d, J=9.9 Hz, 1H), 8.04 (d, J=8.3 Hz, 1H), 7.55 (d, J=1.9 Hz, 1H), 7.52-7.42 (m, 2H), 7.17 (d, J=9.7 Hz, 1H), 4.08 (s, 3H), 3.62 (dt, J=11.9, 6.1 Hz, 2H), 3.49 (q, J=9.5, 8.3 Hz, 1H), 3.36 (q, J=5.6 Hz, 1H), 3.30-3.23 (m, 1H), 3.18-3.05 (m, 1H), 2.32-2.20 (m, 2H), 2.17-2.02 (m, 1H), 1.97 (t, J=7.0 Hz, 2H), 1.91-1.72 (m, 5H), 1.72-1.59 (m, 2H).

### Example 109: Synthesis of Compound 354

### Synthesis of Intermediate B280

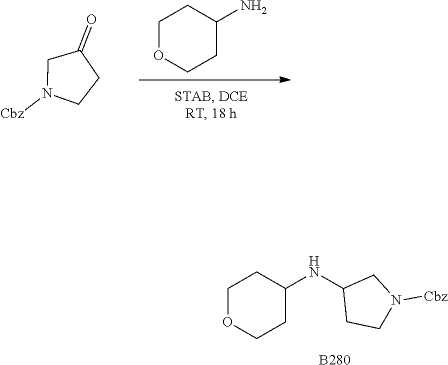

A solution of benzyl 3-oxopyrrolidine-1-carboxylate (1 g, 4.561 mmol, 1 equiv) in DCE (20 mL) was treated with oxan-4-amine (0.55 g, 5.473 mmol, 1.2 equiv) for 2 h at room temperature under nitrogen atmosphere followed by the addition of STAB (2.90 g, 13.683 mmol, 3 equiv) in portions at room temperature. The resulting mixture was stirred for 16 h at room temperature. The reaction was quenched with water at room temperature. The aqueous layer was extracted with CH₂Cl₂ (3×50 mL). The filtrate was concentrated under reduced pressure. The crude product was used in the next step directly without further purification.

### Synthesis of Intermediate B281

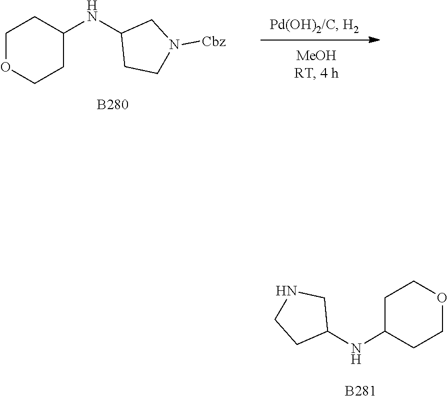

A solution of benzyl 3-(oxan-4-ylamino)pyrrolidine-1-carboxylate (1.9 g, 6.242 mmol, 1 equiv) and Pd(OH)₂/C (600 mg, 4.273 mmol, 0.68 equiv) in MeOH (20 mL) was stirred for 4 h at room temperature under hydrogen atmosphere. The resulting mixture was filtered and the filter cake was washed with MeOH (3×20 mL). The filtrate was concentrated under reduced pressure. The crude product was used in the next step directly without further purification.

### Synthesis of Intermediate B282

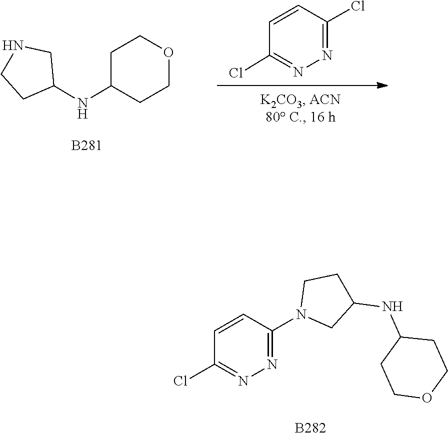

A mixture of N-(oxan-4-yl)pyrrolidin-3-amine (1.09 g, 6.402 mmol, 1 equiv) and pyridazine, 3,6-dichloro- (1.14 g, 7.682 mmol, 1.2 equiv) and K₂CO₃ (2.65 g, 19.206 mmol, 3 equiv) in ACN (20 mL) was stirred for 16 h at 80° C. under nitrogen atmosphere. The resulting mixture was filtered and the filter cake was washed with DCM (30 mL). The filtrate was concentrated under reduced pressure. The residue was purified by silica gel column chromatography, eluted with PE/EA (3:1) to afford 1-(6-chloropyridazin-3-yl)-N-(oxan-4-yl)pyrrolidin-3-amine (1.29 g, 71%).

### Synthesis of Intermediate B283

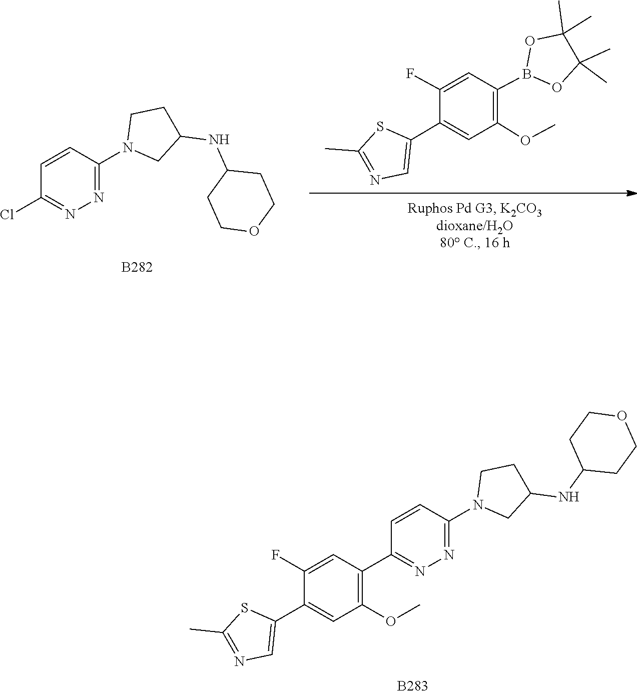

A mixture of 1-(6-chloropyridazin-3-yl)-N-(oxan-4-yl)pyrrolidin-3-amine (150 mg, 0.530 mmol, 1 equiv) and 5-[2-fluoro-5-(methoxymethoxy)-4-(4,4,5,5-tetramethyl-1,3,2-dioxaborolan-2-yl)phenyl]-2-methyl-1,3-thiazole (241.42 mg, 0.636 mmol, 1.2 equiv) and K₂CO₃ (146.63 mg, 1.060 mmol, 2 equiv) and RuPhos Palladacycle Gen.3 (44.37 mg, 0.053 mmol, 0.1 equiv) in 1,4-dioxane was stirred for 16 h at 80° C. under nitrogen atmosphere. The resulting mixture was concentrated under reduced pressure The residue was purified by silica gel column chromatography, eluted with CH₂Cl₂/MeOH (10:1) to afford1-{6-[5-fluoro-2-(methoxymethoxy)-4-(2-methyl-1,3-thiazol-5-yl)phenyl]pyridazin-3-yl}-N-(oxan-4-yl)pyrrolidin-3-amine (90 mg, 34%) as a solid.

### Synthesis of Compound 354

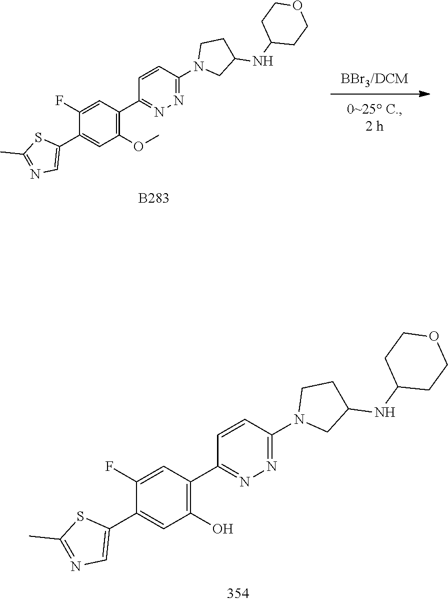 [SMILES: COc1cc(-c2cnc(C)s2)c(F)cc1-c1ccc(N2CCC(NC3CCOCC3)C2)nn1 ; "B283" ; Cc1ncc(-c2cc(O)c(-c3ccc(N4CCC(NC5CCOCC5)C4)nn3)cc2F)s1]

To a stirred solution of 1-{6-[5-fluoro-2-methoxy-4-(2-methyl-1,3-thiazol-5-yl)phenyl]pyridazin-3-yl}-N-(oxan-4-yl)pyrrolidin-3-amine (90 mg, 0.192 mmol, 1 equiv) in DCM (1 mL) was added BBr₃ (0.1 mL, 0.960 mmol, 5 equiv) dropwise portions at 0° C. under nitrogen atmosphere. The resulting mixture was stirred for additional 2 h at room temperature. The reaction was quenched with water/Ice at 0° C. The mixture was neutralized to pH 9 with NH₃·H₂O. The resulting mixture was extracted with EtOAc (3×5 mL). The combined organic layers were dried over anhydrous Na₂SO₄. After filtration, the filtrate was concentrated under reduced pressure. The crude product was purified by Chiral-Prep-HPLC with the following conditions (2SHIMADZU (HPLC-01): Column, Kinetex EVO prep C18, 30×150, 5 um; mobile phase, Water (20 mmol/L NH₄HCO₃) and ACN (15% ACN up to 40% in 10 min); Detector, UV 220 nm) to afford 4-fluoro-5-(2-methyl-1,3-thiazol-5-yl)-2-{6-[3-(oxan-4-ylamino)pyrrolidin-1-yl]pyridazin-3-yl}phenol (0.7 mg, 0.80%) as a solid. LCMS:(ES, m/z): 456 [M+H]^{+ 1}H NMR (400 MHz, DMSO-d₆) δ 13.70 (s, 1H), 8.26 (d, J=9.4 Hz, 1H), 8.16 (s, 1H), 7.94 (d, J=12.6 Hz, 1H), 7.29 (d, J=6.8, Hz, 1H), 7.19 (t, J=8.4 Hz, 1H), 4.45 (s, 1H), 3.57 (s, 2H), 3.50-3.46 (m, 2H), 3.43-3.38 (m, 2H), 3.17-3.15 (m, 1H), 3.12-3.08 (m, 1H), 2.94-2.85 (m, 1H), 2.72 (s, 3H), 2.02-1.90 (m, 2H), 1.88-1.55 (m, 4H).

### Example 110: Synthesis of Compound 363

### Synthesis of Intermediate B284

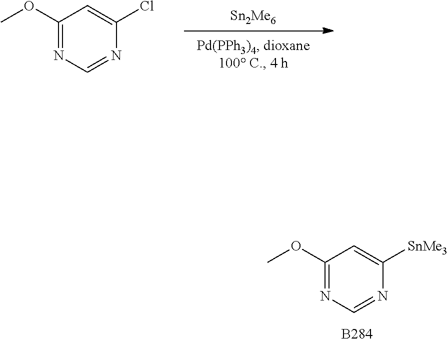

To a stirred mixture of 4-chloro-6-methoxypyrimidine (3 g, 20.753 mmol, 1 equiv) and Pd(PPh₃)₄(1.2 g, 0.1 equiv) in dioxane (70 mL)was added Sn₂Me₆ (8.7 mL, 41.506 mmol, 2 equiv) dropwise at 100° C. for 4 h. Desired product could be detected by LCMS. The aqueous layer was extracted with DCM (100 mL). The resulting mixture was concentrated under reduced pressure to afford 4-methoxy-6-(trimethylstannyl)pyrimidine (5 g, crude) as a solid. The crude product was used in the next step directly without further purification. LCMS:(ES, m/z): 273 [M+H]⁺

### Synthesis of Intermediate B285

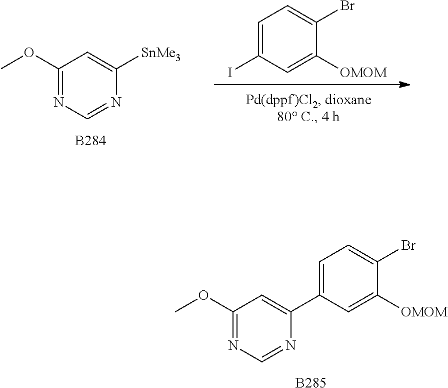

A mixture of 4-methoxy-6-(trimethylstannyl)pyrimidine (1 g, 3.664 mmol, 1 equiv) and 1-bromo-4-iodo-2-(methoxymethoxy)benzene (1.26 g, 3.664 mmol, 1 equiv) in dioxane (12 mL) was stirred for 4 h at 80° C. Desired product could be detected by LCMS. The resulting mixture was concentrated under reduced pressure. The residue was purified by silica gel column chromatography, eluted with PE/EA (1:1) to afford 4-[4-bromo-3-(methoxymethoxy)phenyl]-6-methoxypyrimidine (590 mg, 50%) as a solid. LCMS:(ES, m/z): 326 [M+H]⁺

### Synthesis of Intermediate B286

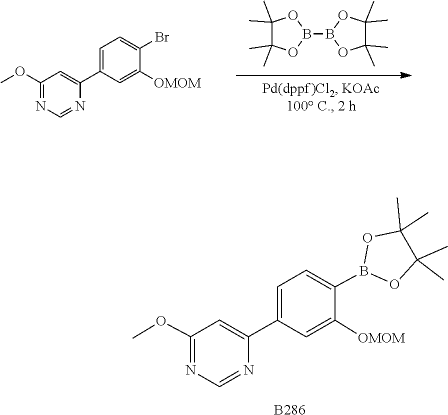

A solution of 4-[4-bromo-3-(methoxymethoxy)phenyl]-6-methoxypyrimidine (1 g, 3.075 mmol, 1 equiv), K₂CO₃ (1.49 g, 10.763 mmol, 3.5 equiv) and bis(pinacolato)diboron (1.56 g, 6.150 mmol, 2 equiv) in dioxane (15 mL) was stirred for 2 h at 100° C. under nitrogen atmosphere. Desired product could be detected by LCMS. The resulting mixture was concentrated under reduced pressure. The residue was purified by silica gel column chromatography, eluted with PE/EA (1:1) to afford 4-methoxy-6-[3-(methoxymethoxy)-4-(4,4,5,5-tetramethyl-1,3,2-dioxaborolan-2-yl)phenyl]pyrimidine (520 mg, 45%) as a solid. LCMS:(ES, m/z): 373 [M+H]⁺

### Synthesis of Intermediate B287

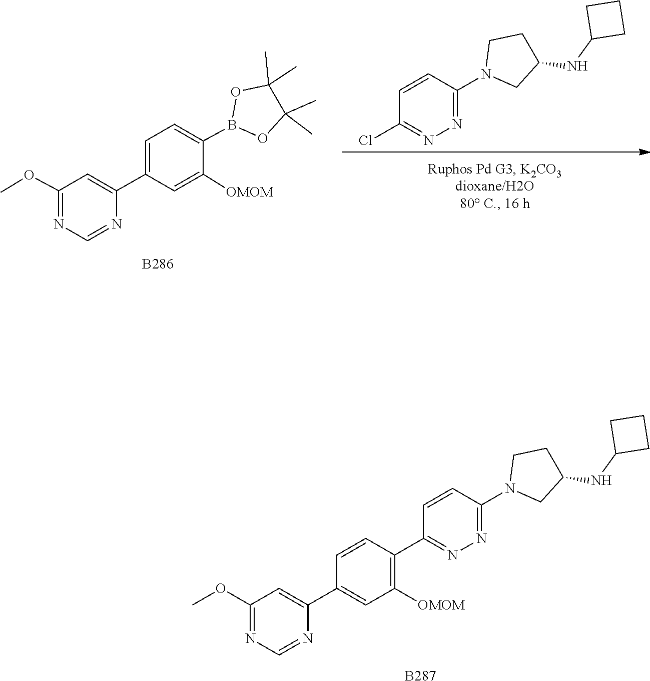

A solution of 4-methoxy-6-[3-(methoxymethoxy)-4-(4,4,5,5-tetramethyl-1,3,2-dioxaborolan-2-yl)phenyl]pyrimidine (107 mg, 0.287 mmol, 1 equiv), (3S)-1-(6-chloropyridazin-3-yl)-N-cyclobutylpyrrolidin-3-amine (94.45 mg, 0.373 mmol, 1.3 equiv), K₂CO₃ (79.46 mg, 0.574 mmol, 2 equiv) and RuPhos Palladacycle Gen.3 (240.42 mg, 0.287 mmol, 1 equiv) in 1,4-dioxane/H₂O(5:1)(6 mL) was stirred for 16 h at 80° C. under nitrogen atmosphere. The resulting mixture was concentrated under reduced pressure. The residue was purified by silica gel column chromatography, eluted with CH₂Cl₂/MeOH (10:1) to afford (3S)—N-cyclobutyl-1-{6-[2-(methoxymethoxy)-4-(6-methoxypyrimidin-4-yl)phenyl]pyridazin-3-yl}pyrrolidin-3-amine (60 mg, 45%) as a solid. LCMS:(ES, m/z): 463 [M+H]⁺

### Synthesis of Compound 363

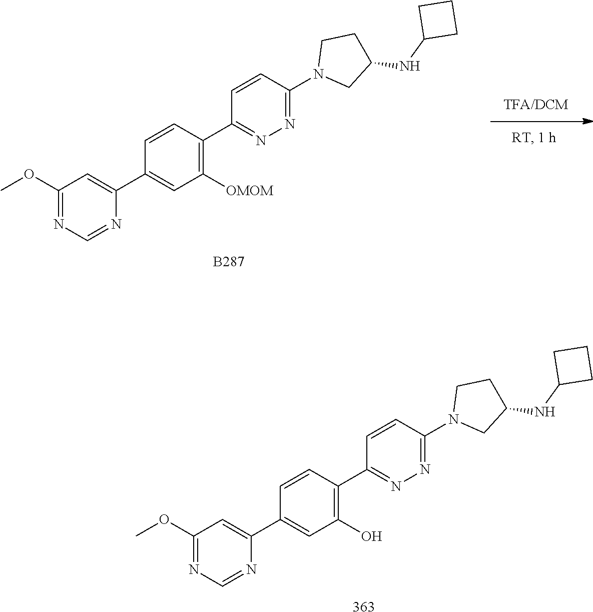

Into a solution of (3S)—N-cyclobutyl-1-{6-[2-(methoxymethoxy)-4-(6-methoxypyrimidin-4-yl)phenyl]pyridazin-3-yl}pyrrolidin-3-amine (60 mg, 0.130 mmol, 1 equiv) in DCM (3 mL) was slowly added TFA (1 mL). Then the solution was stirred at room temperature for 1 h. The resulting mixture was concentrated under reduced pressure. The crude product was purified by Chiral-Prep-HPLC with the following conditions (2SHIMADZU (HPLC-01): Column, Kinetex EVO prep C18, 30×150, 5 um; mobile phase, Water (10 mmol/L NH₄HCO₃) and ACN (15% ACN up to 60% in 10 min); Detector, UV 220 nm) to afford 2-{6-[(3 S)-3-(cyclobutylamino)pyrrolidin-1-yl]pyridazin-3-yl}-5-(6-methoxypyrimidin-4-yl)phenol (2.2 mg, 4%) as a solid. LCMS:(ES, m/z): 418 [M+H]^{+ 1}H NMR: (400 MHz, DMSO-d₆) δ 13.89 (s, 1H), 8.86 (d, J=1.1 Hz, 1H), 8.28 (d, J=9.9 Hz, 1H), 8.02-8.00 (m, 1H), 7.74-7.73 (m, 2H), 7.52 (d, J=1.1 Hz, 1H), 7.17 (d, J=9.7 Hz, 1H), 3.99 (s, 3H), 3.68-3.60 (m, 2H), 3.52 (t, J=7.7 Hz, 1H), 3.41 (s, 1H), 3.30-3.28 (m, 2H), 2.15 (s, 3H), 2.19-2.06 (m, 1H), 1.89-1.84 (m, 2H), 1.72-1.60 (m, 2H).

### Example 111: Synthesis of Compound 364

### Synthesis of Intermediate B288

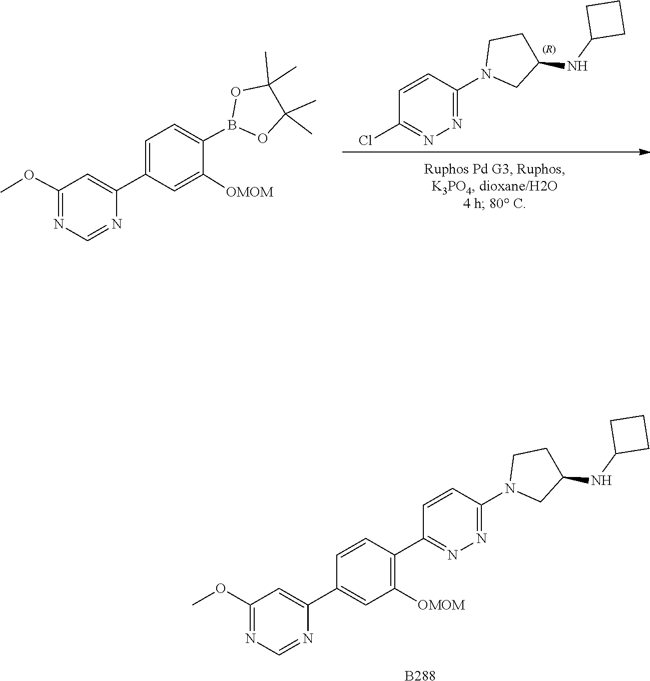

To a solution of 4-methoxy-6-[3-(methoxymethoxy)-4-(4,4,5,5-tetramethyl-1,3,2-dioxaborolan-2-yl)phenyl]pyrimidine (100 mg, 0.269 mmol, 1 equiv) and (3R)-1-(6-chloropyridazin-3-yl)-N-cyclobutylpyrrolidin-3-amine (47.53 mg, 0.188 mmol, 0.7 equiv) in dioxane (1 mL) and water (0.2 mL) were added K₃PO₄ (142.56 mg, 0.673 mmol, 2.5 equiv) and RuPhos Palladacycle Gen.3 (11.23 mg, 0.013 mmol, 0.05 equiv) and RuPhos (12.54 mg, 0.027 mmol, 0.1 equiv). After stirring for 4 h at 80° C. under a nitrogen atmosphere. The mixture was allowed to cool down to room temperature, the resulting mixture was concentrated under reduced pressure. The reaction was quenched by the addition of Water (5 mL) at room temperature. The aqueous layer was extracted with EtOAc (3×10 mL). The resulting mixture was concentrated under reduced pressure. The residue was dissolved in CH₂Cl₂ (5 mL). The residue was purified by silica gel column chromatography, eluted with CH₂Cl₂/MeOH (5:1) to afford (3R)—N-cyclobutyl-1-{6-[2-(methoxymethoxy)-4-(6-methoxypyrimidin-4-yl)phenyl]pyridazin-3-yl}pyrrolidin-3-amine (50 mg, 40%) as an oil. LCMS:(ES, m/z):463.05[M+H]

### Synthesis of Compound 364

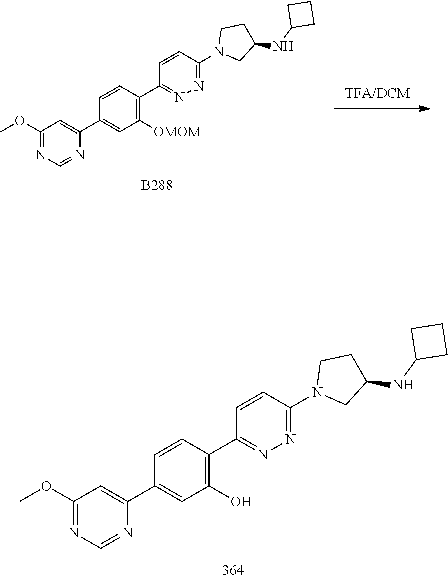

To a stirred solution of (3R)—N-cyclobutyl-1-{6-[2-(methoxymethoxy)-4-(6-methoxypyrimidin-4-yl)phenyl]pyridazin-3-yl}pyrrolidin-3-amine (50 mg, 0.108 mmol, 1 equiv) in DCM (2 mL) was added TFA (1 mL) dropwise at 0° C. under air atmosphere. The solution was stirred for 1 h at room temperature under air atmosphere. The reaction was quenched with MeOH at 0° C. The crude product (50 mg) was purified by Prep-HPLC with the following conditions (Column, Kinetex EVO prep C18, 30×150, 5 um; mobile phase, Water (10 mmol/L NH₄HCO₃) and ACN (15% ACN up to 55% in 10 min)) to afford 2-{6-[(3R)-3-(cyclobutylamino)pyrrolidin-1-yl]pyridazin-3-yl}-5-(6-methoxypyrimidin-4-yl)phenol (20.1 mg, 44%) as a solid.

LCMS:(ES, m/z):418.95[M+H]⁺¹H NMR:(400 MHz, DMSO-d₆) δ 13.90 (s, 1H), 8.86 (d, J=1.1 Hz, 1H), 8.28 (d, J=9.8 Hz, 1H), 8.05-7.98 (m, 1H), 7.77-7.70 (m, 2H), 7.52 (d, J=1.1 Hz, 1H), 7.16 (d, J=9.8 Hz, 1H), 3.99 (s, 3H), 3.70-3.57 (m, 2H), 3.52 (d, J=7.1 Hz, 1H), 3.48 (d, J=9.1 Hz, 1H), 3.40 (p, J=5.9 Hz, 1H), 3.24 (d, J=7.9 Hz, 1H), 2.20-2.04 (m, 3H), 1.83 (dq, J=12.9, 6.6 Hz, 1H), 1.69 (s, 2H), 1.62 (td, J=28.7, 25.7, 20.9, 10.3, 7.3 Hz, 2H).

### Example 112: Synthesis of Compounds 365 and 366

### Synthesis of Intermediates B289

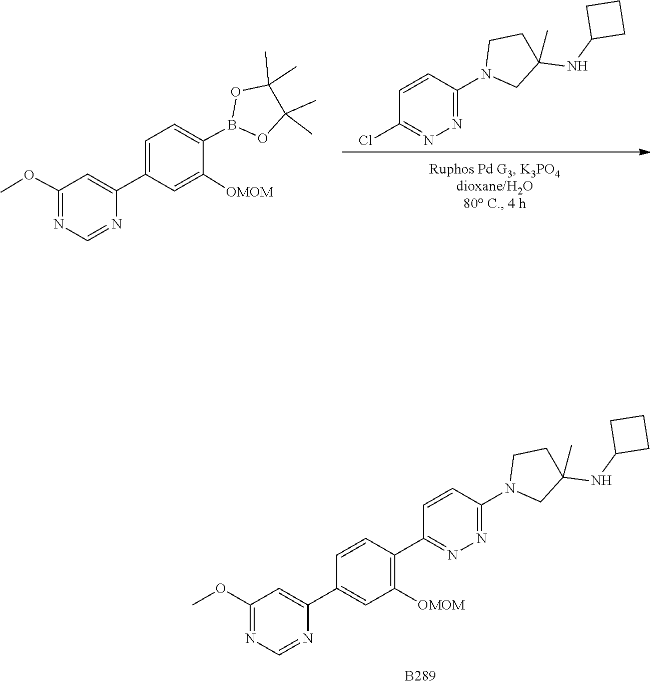

To a solution of 4-methoxy-6-[3-(methoxymethoxy)-4-(4,4,5,5-tetramethyl-1,3,2-dioxaborolan-2-yl)phenyl]pyrimidine (300 mg, 0.806 mmol, 1 equiv) and (3S)-1-(6-chloropyridazin-3-yl)-N-cyclobutyl-3-methylpyrrolidin-3-amine (150.5 mg, 0.564 mmol, 0.7 equiv) in dioxane (5 mL) and water (1 mL) were added K₃PO₄ (513.23 mg, 2.418 mmol, 3 equiv) and RuPhos Palladacycle Gen.3 (18.8 mg, 0.040 mmol, 0.05 equiv) and RuPhos (37.61 mg, 0.081 mmol, 0.1 equiv). After stirring for 4 h at 80° C. under a nitrogen atmosphere. The mixture was allowed to cool down to room temperature, the resulting mixture was concentrated under reduced pressure. The reaction was quenched by the addition of Water (5 mL) at room temperature. The aqueous layer was extracted with EtOAc (3×10 mL). The resulting mixture was concentrated under reduced pressure. The residue was dissolved in CH₂Cl₂ (5 mL). The residue was purified by silica gel column chromatography, eluted with CH₂Cl₂/MeOH (5:1) to afford (3S)—N-cyclobutyl-1-{6-[2-(methoxymethoxy)-4-(6-methoxypyrimidin-4-yl)phenyl]pyridazin-3-yl}-3-methylpyrrolidin-3-amine (115 mg, 30%) as a solid. LCMS:(ES, m/z):477.15[M+H]

### Synthesis of Compounds 365 and 366

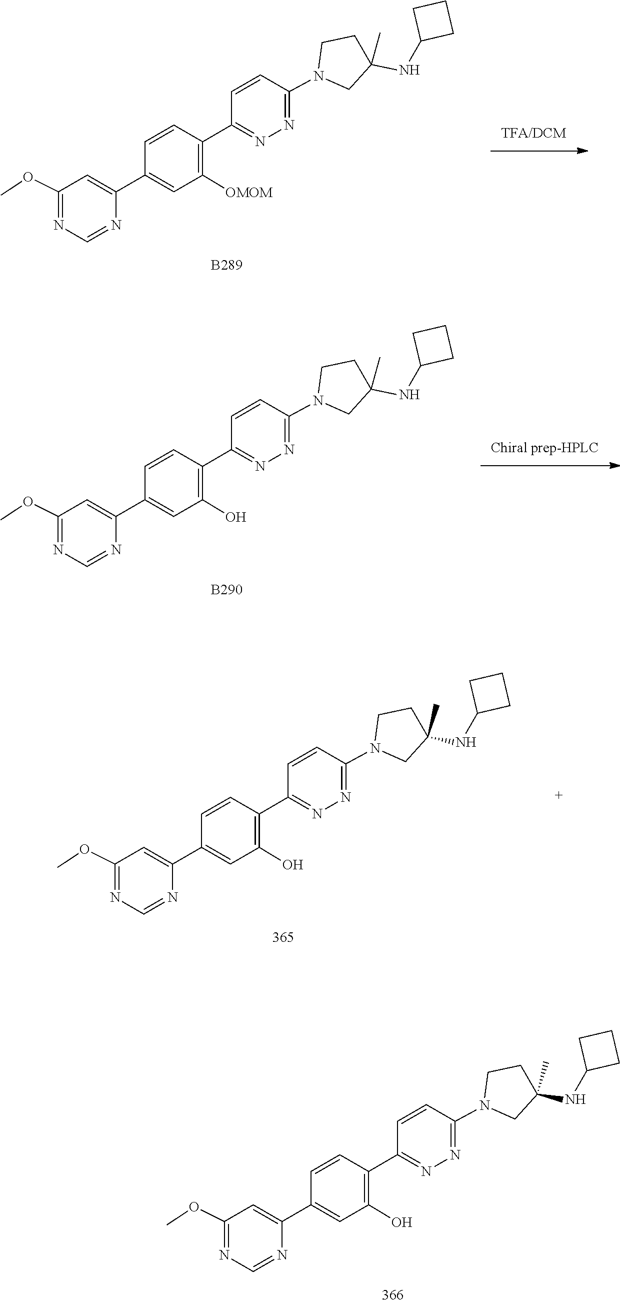

To a stirred solution of (3S)—N-cyclobutyl-1-{6-[2-(methoxymethoxy)-4-(6-methoxypyrimidin-4-yl)phenyl]pyridazin-3-yl}-3-methylpyrrolidin-3-amine (50 mg, 0.105 mmol, 1 equiv) in DCM (2.5 mL) was added TFA (0.5 mL) dropwise at 0° C. under air atmosphere. The reaction was quenched by the addition of MeOH (5 mL) at 0° C. The crude product (50 mg) was purified by Prep-Chiral-HPLC with the following conditions (Column: CHIRAL ART Cellulose-SB, 2*25 cm, 5 m; Mobile Phase A: Hex(0.1% DEA)-HPLC, Mobile Phase B: EtOH-HPLC; Flow rate: 20 mL/min; Gradient: 50% B to 50% B in 13 min; Wave Length: 254/220 nm; RT1(min): 9.54; RT2(min): 11.44; Sample Solvent: MeOH:DCM=1:2; Injection Volume: 0.6 mL; Number Of Runs: 11) to afford 2-{6-[(3S)-3-(cyclobutylamino)-3-methylpyrrolidin-1-yl]pyridazin-3-yl}-5-(6-methoxypyrimidin-4-yl)phenol (5.3 mg, 11.68%) as a yellow solid and 2-{6-[(3R)-3-(cyclobutylamino)-3-methylpyrrolidin-1-yl]pyridazin-3-yl}-5-(6-methoxypyrimidin-4-yl)phenol (5.9 mg, 12%) as a solid. Compound 365: LCMS:(ES, m/z): 432.90[M+H]¹HNMR: (400 MHz, DMSO-d₆) δ 13.89 (s, 1H), 8.86 (d, J=1.1 Hz, 1H), 8.27 (d, J=9.8 Hz, 1H), 8.01 (d, J=8.8 Hz, 1H), 7.77-7.70 (m, 2H), 7.52 (d, J=1.1 Hz, 1H), 7.15 (d, J=9.7 Hz, 1H), 3.99 (s, 3H), 3.68 (s, 1H), 3.65 (s, 1H), 3.63 (s, 1H), 3.58 (s, 1H), 3.55 (d, J=7.9 Hz, 1H), 2.48 (s, 1H), 2.13 (ddt, J=10.1, 7.0, 3.2 Hz, 2H), 2.00 (dt, J=13.4, 7.1 Hz, 1H), 1.89-1.81 (m, 1H), 1.76 (dt, J=26.9, 7.5 Hz, 3H), 1.55 (dq, J=10.5, 4.4, 3.8 Hz, 2H), 1.23 (s, 3H). Compound 366: LCMS:(ES, m/z):432.90[M+H] ¹H NMR: (400 MHz, DMSO-d₆) δ 13.89 (s, 1H), 8.86 (d, J=1.1 Hz, 1H), 8.27 (d, J=9.8 Hz, 1H), 8.01 (d, J=8.8 Hz, 1H), 7.77-7.70 (m, 2H), 7.52 (d, J=1.1 Hz, 1H), 7.15 (d, J=9.7 Hz, 1H), 3.99 (s, 3H), 3.68 (s, 1H), 3.65 (s, 1H), 3.63 (s, 1H), 3.58 (s, 1H), 3.55 (d, J=7.9 Hz, 1H), 2.48 (s, 1H), 2.13 (ddt, J=10.1, 7.0, 3.2 Hz, 2H), 2.00 (dt, J=13.4, 7.1 Hz, 1H), 1.89-1.81 (m, 1H), 1.76 (dt, J=26.9, 7.5 Hz, 3H), 1.55 (dq, J=10.5, 4.4, 3.8 Hz, 2H), 1.23 (s, 3H).

### Example 113: Synthesis of Compounds 375 and 376

### Synthesis of Intermediate B291

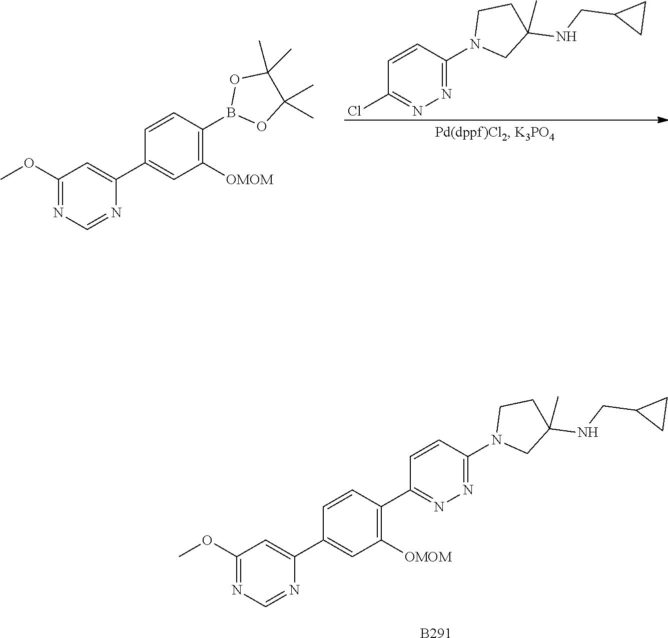

To a solution of 4-methoxy-6-[3-(methoxymethoxy)-4-(4,4,5,5-tetramethyl-1,3,2-dioxaborolan-2-yl)phenyl]pyrimidine (200 mg, 0.537 mmol, 1 equiv) and 1-(6-chloropyridazin-3-yl)-N-(cyclopropylmethyl)-3-methylpyrrolidin-3-amine (100.34 mg, 0.376 mmol, 0.7 equiv) in dioxane (5 mL) and H₂O (1 mL) were added K₃PO₄ (342.15 mg, 1.611 mmol, 3 equiv) and Pd(dppf)Cl₂ (39.31 mg, 0.054 mmol, 0.1 equiv). After stirring for 4 h at 80° C. under a nitrogen atmosphere, the resulting mixture was concentrated under reduced pressure. The resulting mixture was extracted with EtOAc (3×10 mL). The combined organic layers were washed with water (3×10 mL), dried over anhydrous Na₂SO₄. After filtration, the filtrate was concentrated under reduced pressure. The residue was purified by silica gel column chromatography, eluted with CH₂Cl₂/MeOH (5:1) to afford N-(cyclopropylmethyl)-1-{6-[2-(methoxymethoxy)-4-(6-methoxypyrimidin-4-yl)phenyl]pyridazin-3-yl}-3-methylpyrrolidin-3-amine (100 mg, 39%) as an oil. LCMS:(ES, m/z):476.25[M+H]

### Synthesis of Compounds 375 and 376

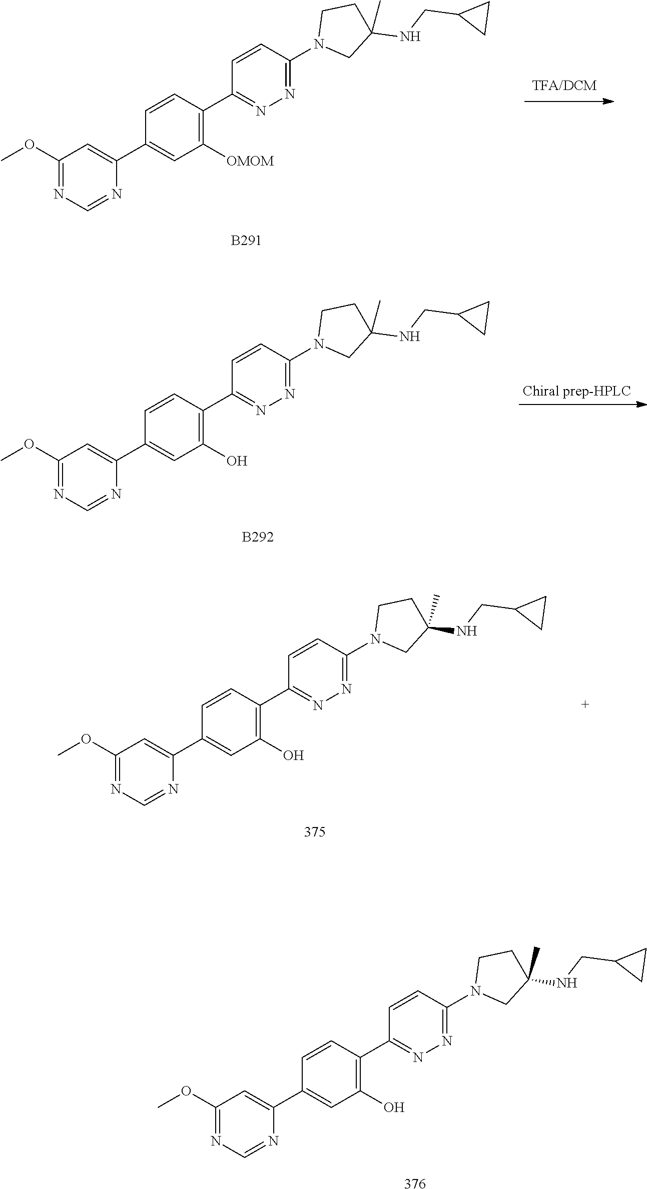

To a stirred solution of (3R)—N-(cyclopropylmethyl)-1-{6-[2-(methoxymethoxy)-4-(6-methoxypyrimidin-4-yl)phenyl]pyridazin-3-yl}-3-methylpyrrolidin-3-amine (30 mg, 0.063 mmol, 1 equiv) in DCM (1 mL) was added TFA (0.5 mL) dropwise at 0° C. under air atmosphere. The reaction was monitored by LCMS. The crude product (20 mg) was purified by Prep-HPLC with the following conditions (Column, CHIRALPAK IG, 2*25 cm, 5 um; mobile phase, MtBE(0.1% DEA) and MeOH- (hold 50% MeOH- in 15 min)) to afford 2-{6-[(3R)-3-[(cyclopropylmethyl)amino]-3-methylpyrrolidin-1-yl]pyridazin-3-yl}-5-(6-methoxypyrimidin-4-yl)phenol (6.4 mg, 23.04%) as a yellow solid and 2-{6-[(3S)-3-[(cyclopropylmethyl)amino]-3-methylpyrrolidin-1-yl]pyridazin-3-yl}-5-(6-methoxypyrimidin-4-yl)phenol (6.4 mg, 21.23%) as a solid. Compound 375: LCMS:(ES, m/z):432.85[M+H]¹HNMR: (400 MHz, DMSO-d₆) δ 13.90 (s, 1H), 8.86 (d, J=1.0 Hz, 1H), 8.27 (d, J=9.8 Hz, 1H), 8.04-7.98 (m, 1H), 7.77-7.70 (m, 2H), 7.52 (d, J=1.4 Hz, 1H), 7.15 (d, J=9.7 Hz, 1H), 3.99 (s, 3H), 3.68-3.60 (m, 2H), 3.57 (d, J=8.2 Hz, 1H), 3.49 (d, J=10.7 Hz, 1H), 2.48-2.37 (m, 2H), 2.03 (ddd, J=13.5, 7.7, 5.8 Hz, 1H), 1.84 (dt, J=12.2, 7.3 Hz, 1H), 1.23 (s, 3H), 0.90-0.78 (m, 1H), 0.43-0.32 (m, 2H), 0.15-0.03 (m, 2H). Compound 376: LCMS:(ES, m/z):432.90[M+H]¹HNMR: (400 MHz, DMSO-d₆) δ 13.90 (s, 1H), 8.86 (d, J=1.0 Hz, 1H), 8.27 (d, J=9.8 Hz, 1H), 8.04-7.98 (m, 1H), 7.77-7.70 (m, 2H), 7.52 (d, J=1.4 Hz, 1H), 7.15 (d, J=9.7 Hz, 1H), 3.99 (s, 3H), 3.68-3.60 (m, 2H), 3.57 (d, J=8.2 Hz, 1H), 3.49 (d, J=10.7 Hz, 1H), 2.48-2.37 (m, 2H), 2.03 (ddd, J=13.5, 7.7, 5.8 Hz, 1H), 1.84 (dt, J=12.2, 7.3 Hz, 1H), 1.23 (s, 3H), 0.90-0.78 (m, 1H), 0.43-0.32 (m, 2H), 0.15-0.03 (m, 2H).

### Example 114: Synthesis of Compound 379

### Synthesis of Intermediate B293

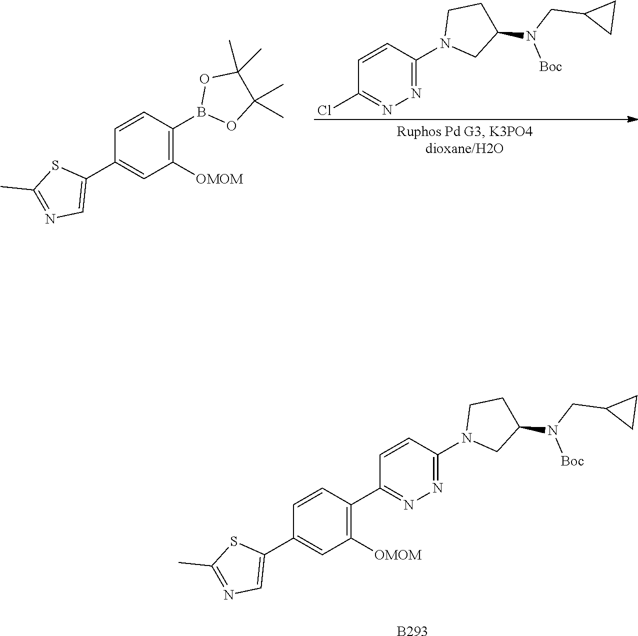

To a solution of 5-[3-(methoxymethoxy)-4-(4,4,5,5-tetramethyl-1,3,2-dioxaborolan-2-yl)phenyl]-2-methyl-1,3-thiazole (100 mg, 0.277 mmol, 1.5 equiv) and tert-butyl N-[(3R)-1-(6-chloropyridazin-3-yl)pyrrolidin-3-yl]-N-(cyclopropylmethyl)carbamate (65.12 mg, 0.185 mmol, 1 equiv) in dioxane (2 mL) and water (0.4 mL) were added K₃PO₄ (117.51 mg, 0.554 mmol, 3 equiv) and RuPhos Palladacycle Gen.3 (15.43 mg, 0.018 mmol, 0.1 equiv). After stirring for 4 h at 80° C. under a nitrogen atmosphere, the resulting mixture was concentrated under reduced pressure. The residue was purified by silica gel column chromatography, eluted with CH₂Cl₂/MeOH (5:1) to afford tert-butyl N-(cyclopropylmethyl)-N-[(3R)-1-{6-[2-(methoxymethoxy)-4-(2-methyl-1,3-thiazol-5-yl)phenyl]pyridazin-3-yl}pyrrolidin-3-yl]carbamate as a solid. LCMS:(ES, m/z): 551.90[M+H]

### Synthesis of Compound 379

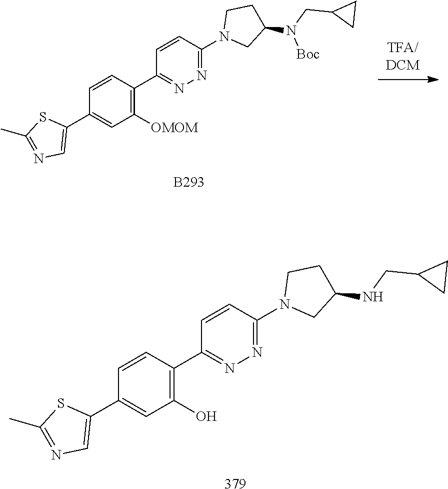

To a stirred solution of tert-butyl N-(cyclopropylmethyl)-N-[(3R)-1-{6-[2-(methoxymethoxy)-4-(2-methyl-1,3-thiazol-5-yl)phenyl]pyridazin-3-yl}pyrrolidin-3-yl]carbamate (100 mg, 0.181 mmol, 1 equiv) in DCM (2 mL) was added TFA (1 mL) dropwise at 0° C. under air atmosphere. The resulting mixture was stirred for 1 h at room temperature under air atmosphere. The reaction was quenched with MeOH at 0° C. The resulting mixture was concentrated under reduced pressure. The crude product (100 mg) was purified by Prep-HPLC with the following conditions (Column, Kinetex EVO prep C18, 30×150, 5 um; mobile phase, Water (10 mmol/L NH₄HCO₃) and ACN (15% ACN up to 57% in 8 min)) to afford 2-{6-[(3R)-3-[(cyclopropylmethyl)amino]pyrrolidin-1-yl]pyridazin-3-yl}-5-(2-methyl-1,3-thiazol-5-yl)phenol (22.2 mg, 30%) as a solid. LCMS:(ES, m/z):407.85[M+H] ¹H NMR:(400 MHz, DMSO-d₆) δ 14.03 (s, 1H), 8.23 (d, J=9.9 Hz, 1H), 8.10 (s, 1H), 7.92 (d, J=8.8 Hz, 1H), 7.17 (td, J=4.1, 1.6 Hz, 3H), 3.68 (dd, J=10.8, 6.0 Hz, 1H), 3.61 (t, J=7.6 Hz, 1H), 3.51 (m, 1H), 3.50 (m, 1H), 3.48 (m, 1H), 2.68 (s, 3H), 2.46 (d, J=6.7 Hz, 2H), 2.14 (dq, J=13.1, 6.8 Hz, 1H), 1.88 (dd, J=13.2, 6.9 Hz, 1H), 0.89 (m, 1H), 0.46-0.37 (m, 2H), 0.17-0.09 (m, 2H).

### Example 115: Synthesis of Compound 380

### Synthesis of Intermediate B294

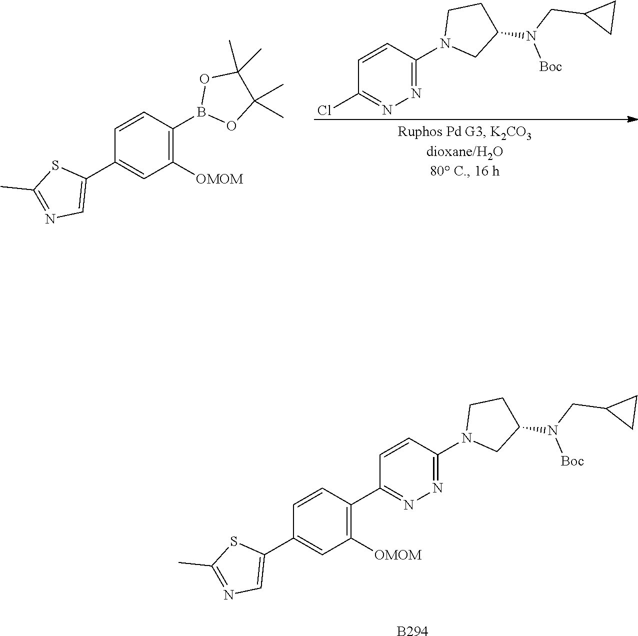

A solution of 5-[3-(methoxymethoxy)-4-(4,4,5-trimethyl-1,3,2-dioxaborolan-2-yl)phenyl]-2-methyl-1,3-thiazole (100 mg, 0.288 mmol, 1 equiv), tert-butyl N-[(3S)-1-(6-chloropyridazin-3-yl)pyrrolidin-3-yl]-N-(cyclopropylmethyl)carbamate (111.78 mg, 0.317 mmol, 1.1 equiv), RuPhos Palladacycle Gen.3 (24.09 mg, 0.029 mmol, 0.1 equiv) and K₂CO₃ (79.6 mg, 0.576 mmol, 2 equiv) in 1,4-dioxane/H₂O(5:1)(2.4 mL) was stirred for 16 h at 80° C. under nitrogen atmosphere. The residue was purified by silica gel column chromatography, eluted with PE/EA (0%˜90%) to afford tert-butyl N-(cyclopropylmethyl)-N-[(3S)-1-{6-[2-(methoxymethoxy)-4-(2-methyl-1,3-thiazol-5-yl)phenyl]pyridazin-3-yl}pyrrolidin-3-yl]carbamate (110 mg, 69%) as a solid. LCMS:(ES, m/z): 552 [M+H]⁺

### Synthesis of Compound 380

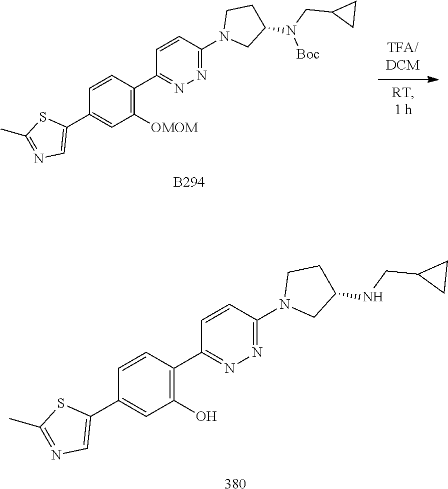

A solution of tert-butyl N-(cyclopropylmethyl)-N-[(3S)-1-{6-[2-(methoxymethoxy)-4-(2-methyl-1,3-thiazol-5-yl)phenyl]pyridazin-3-yl}pyrrolidin-3-yl]carbamate (100 mg, 0.181 mmol, 1 equiv) and TFA (0.1 mL, 1.346 mmol, 7.43 equiv) in DCM (1.5 mL) was stirred for 1 h at 25° C. The resulting mixture was concentrated under reduced pressure. The crude product was purified by Chiral-Prep-HPLC with the following conditions (2SHIMADZU (HPLC-01): Column, Kinetex EVO prep C18, 30×150, 5 um; mobile phase, Water (10 mmol/L NH₄HCO₃) and ACN (15% ACN up to 55% in 10 min); Detector, UV 220 nm) to afford 2-{6-[(3S)-3-[(cyclopropylmethyl)amino]pyrrolidin-1-yl]pyridazin-3-yl}-5-(2-methyl-1,3-thiazol-5-yl)phenol (33.2 mg, 43%) as a solid. LCMS:(ES, m/z): 408 [M+H]⁺¹H NMR: (400 MHz, DMSO-d₆) δ 14.02 (s, 1H), 8.22 (d, J=9.9 Hz, 1H), 8.10 (s, 1H), 7.92 (d, J=8.8 Hz, 1H), 7.17 (t, J=4.0 Hz, 3H), 3.70-3.65 (m, 1H), 3.55-3.46 (m, 1H), 3.36-3.31 (m, 2H), 3.28 (s, 1H), 2.68 (s, 3H), 2.47 (d, J=6.7 Hz, 2H), 2.14 (dq, J=13.1, 6.7 Hz, 1H), 1.89 (dt, J=12.7, 6.3 Hz, 1H), 0.42-0.40 (m, 2H), 0.14-0.12 (m, 2H).

### Example 116: Synthesis of Compound 381

### Synthesis of Intermediate B295

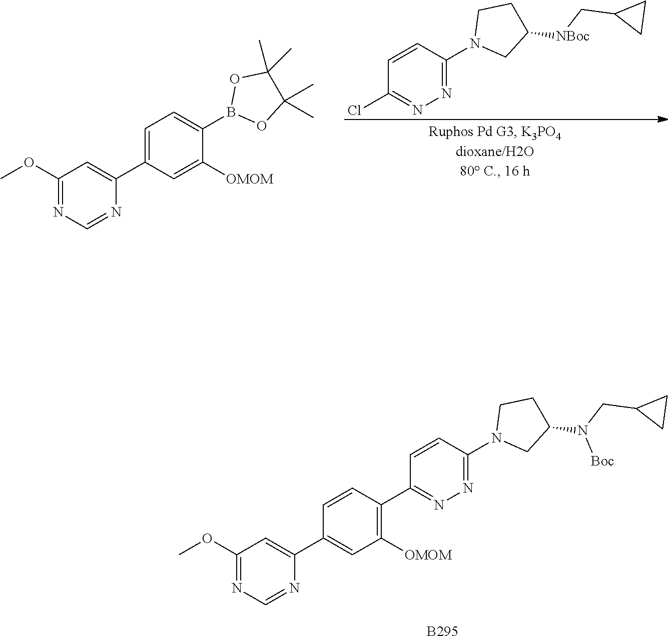

A mixture of 4-methoxy-6-[3-(methoxymethoxy)-4-(4,4,5-trimethyl-1,3,2-dioxaborolan-2-yl)phenyl]pyrimidine (100 mg, 0.279 mmol, 1 equiv) and tert-butyl N-[(3S)-1-(6-chloropyridazin-3-yl)pyrrolidin-3-yl]-N-(cyclopropylmethyl)carbamate (108.36 mg, 0.307 mmol, 1.1 equiv) and RuPhos Palladacycle Gen.3 (23.35 mg, 0.028 mmol, 0.1 equiv) and K₃PO₄ (118.52 mg, 0.558 mmol, 2 equiv) in dioxane (2 mL) and H₂O (0.4 mL) was stirred for 16 h at 80° C. under nitrogen atmosphere. The resulting mixture was dried over anhydrous Na₂SO₄. After filtration, the filtrate was concentrated under reduced pressure. The residue was purified by silica gel column chromatography, eluted with PE/EA (0%˜90%) to afford tert-butyl N-(cyclopropylmethyl)-N-[(3S)-1-{6-[2-(methoxymethoxy)-4-(6-methoxypyrimidin-4-yl)phenyl]pyridazin-3-yl}pyrrolidin-3-yl]carbamate (100 mg, 64%) as a solid. LCMS:(ES, m/z): 563 [M+H]⁺

### Synthesis of Compound 381

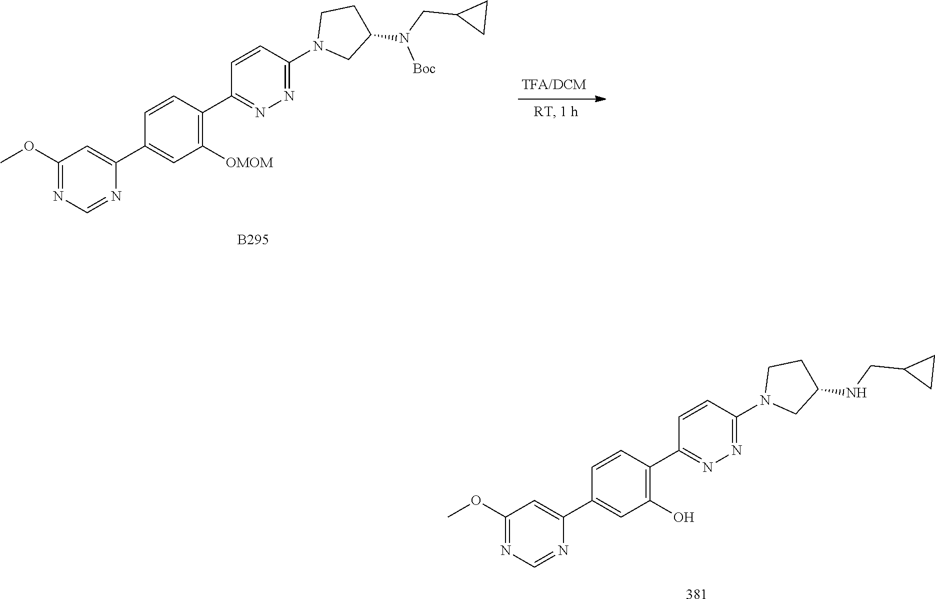

A solution of tert-butyl N-(cyclopropylmethyl)-N-[(3S)-1-{6-[2-(methoxymethoxy)-4-(6-methoxypyrimidin-4-yl)phenyl]pyridazin-3-yl}pyrrolidin-3-yl]carbamate (100 mg, 0.178 mmol, 1 equiv) and TFA (1 mL, 13.463 mmol, 75.75 equiv) in DCM (3 mL) was stirred for 1 h at 25° C. The resulting mixture was concentrated under reduced pressure. The crude product was purified by Chiral-Prep-HPLC with the following conditions (2SHIMADZU (HPLC-01)): Column, XBridge Shield RP18 OBD Column, 30×150 mm, 5 μm; mobile phase, Water (10 mmol/L NH₄HCO₃) and ACN (20% ACN up to 50% in 10 min); Detector, UV220 nm) to afford 2-{6-[(3S)-3-[(cyclopropylmethyl)amino]pyrrolidin-1-yl]pyridazin-3-yl}-5-(6-methoxypyrimidin-4-yl)phenol (13.8 mg, 18%) as a solid. LCMS: (ES, m/z): 419 [M+H]^{+ 1}H NMR (400 MHz, DMSO-d₆) δ 13.90 (s, 1H), 8.87 (s, 1H), 8.29 (d, J=9.8 Hz, 1H), 8.02 (d, J=8.9 Hz, 1H), 7.77-7.71 (m, 2H), 7.53 (s, 1H), 7.18 (d, J=9.8 Hz, 1H), 3.99 (s, 3H), 3.70 (s, 1H), 3.63 (s, 1H), 3.53 (s, 2H), 3.28 (s, 1H), 2.67-2.51 (m, 2H), 2.15 (s, 1H), 1.90 (s, 1H), 0.89 (s, 1H), 0.42 (d, J=7.8 Hz, 2H), 0.14 (d, J=4.2 Hz, 2H).

### Example 117: Synthesis of Compound 382

### Synthesis of Intermediate B296

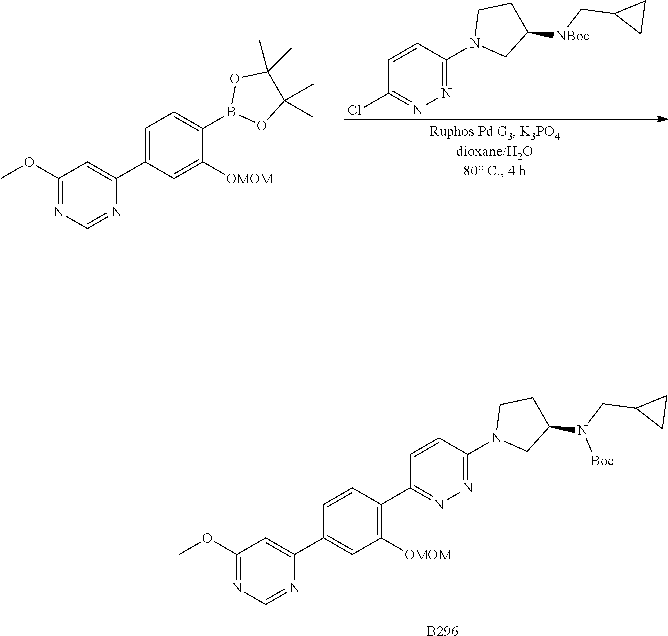

To a solution of 4-methoxy-6-[3-(methoxymethoxy)-4-(4,4,5,5-tetramethyl-1,3,2-dioxaborolan-2-yl)phenyl]pyrimidine (20 mg, 0.054 mmol, 1 equiv) and tert-butyl N-[(3R)-1-(6-chloropyridazin-3-yl)pyrrolidin-3-yl]-N-(cyclopropylmethyl)carbamate (13.27 mg, 0.038 mmol, 0.7 equiv) in dioxane (1 mL) and water (0.2 mL) were added K₃PO₄ (34.22 mg, 0.162 mmol, 3 equiv) and RuPhos Palladacycle Gen.3 (4.49 mg, 0.005 mmol, 0.1 equiv). After stirring for 4 h at 80° C. under a nitrogen atmosphere. The mixture was allowed to cool down to room temperature, the resulting mixture was concentrated under reduced pressure. The resulting mixture was extracted with CH₂Cl₂ (2×10 mL). The combined organic layers were washed with water (2×10 mL), dried over anhydrous Na₂SO₄. After filtration, the filtrate was concentrated under reduced pressure. The residue was purified by silica gel column chromatography, eluted with CH₂Cl₂/MeOH (5:1) to afford tert-butyl N-(cyclopropylmethyl)-N-[(3R)-1-{6-[2-hydroxy-4-(6-methoxypyrimidin-4-yl)phenyl]pyridazin-3-yl}pyrrolidin-3-yl]carbamate as a solid. LCMS:(ES, m/z):563.15[M+H]

### Synthesis of Compound 382

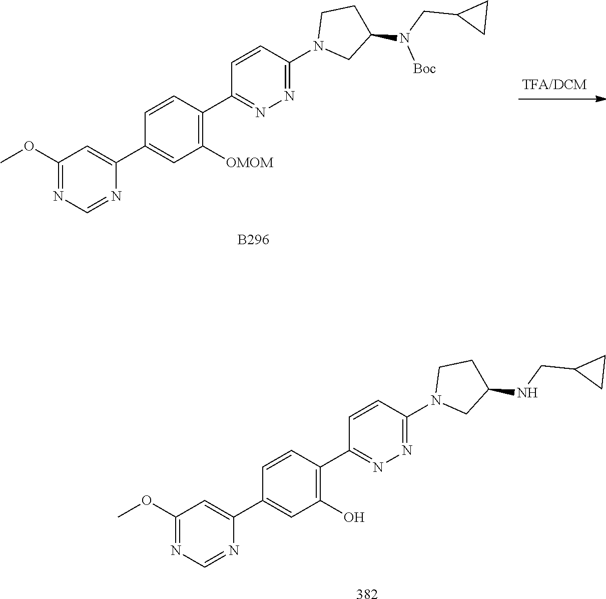

A solution of tert-butyl N-(cyclopropylmethyl)-N-[(3R)-1-{6-[2-(methoxymethoxy)-4-(6-methoxypyrimidin-4-yl)phenyl]pyridazin-3-yl}pyrrolidin-3-yl]carbamate (100 mg, 0.178 mmol, 1 equiv) and TFA (1 mL) in DCM (2 mL) was stirred for 1 h at room temperature under air atmosphere. The reaction was quenched by the addition of MeOH (5 mL) at 0° C. The resulting mixture was concentrated under reduced pressure. The crude product (100 mg) was purified by Prep-HPLC with the following conditions (Column, Kinetex EVO prep C18, 30×150, 5 um; mobile phase, Water (10 mmol/L NH₄HCO₃) and ACN (20% ACN up to 48% in 10 min)) to afford 2-{6-[(3R)-3-[(cyclopropylmethyl)amino]pyrrolidin-1-yl]pyridazin-3-yl}-5-(6-methoxypyrimidin-4-yl)phenol (8.8 mg, 12%) as a solid. LCMS:(ES, m/z):418.90[M+H]¹HNMR:(400 MHz, DMSO-d₆) δ 13.90 (s, 1H), 8.87 (d, J=1.0 Hz, 1H), 8.29 (d, J=9.8 Hz, 1H), 8.02 (d, J=9.0 Hz, 1H), 7.77-7.71 (m, 2H), 7.53 (d, J=1.1 Hz, 1H), 7.18 (d, J=9.7 Hz, 1H), 3.99 (s, 3H), 3.70 (dd, J=10.8, 5.9 Hz, 1H), 3.67 (s, 1H), 3.65 (m, 2H), 3.62 (s, 1H), 3.55 (s, 1H), 3.52 (s, 1H), 2.16 (dt, J 12.6, 6.7 Hz, 1H), 1.95-1.87 (m, 1H), 0.47-0.38 (m, 2H), 0.18-0.10 (m, 2H).

### Example 118: Synthesis of Compounds 383 and 384

### Synthesis of Intermediate B297

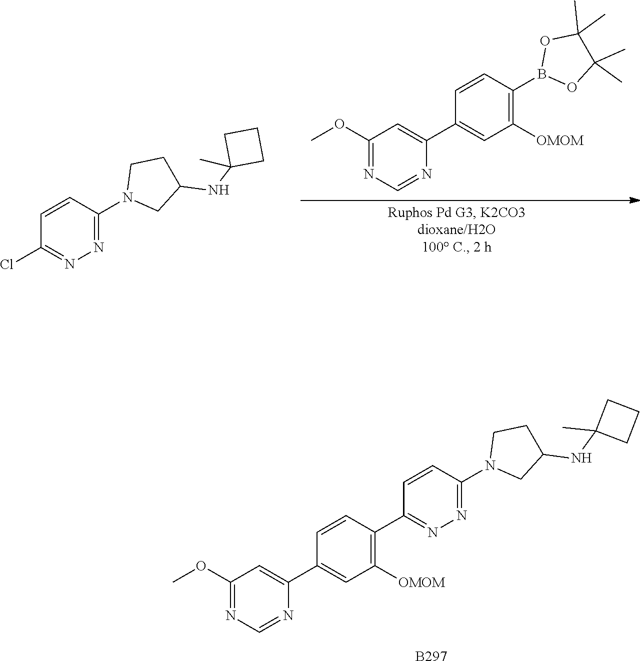

A solution of 1-(6-chloropyridazin-3-yl)-N-(1-methylcyclobutyl)pyrrolidin-3-amine (200 mg, 0.750 mmol, 1 equiv) in dioxane (4 mL, 47.216 mmol, 62.98 equiv)/H₂O (1 mL, 55.509 mmol, 74.04 equiv) was treated with 4-methoxy-6-[3-(methoxymethoxy)-4-(4,4,5,5-tetramethyl-1,3,2-dioxaborolan-2-yl)phenyl]pyrimidine (279.06 mg, 0.750 mmol, 1 equiv), K₂CO₃ (310.84 mg, 2.250 mmol, 3 equiv) and RuPhos Palladacycle Gen.3 (62.7 mg, 0.075 mmol, 0.1 equiv) for 2 h at 100° C. under nitrogen atmosphere. The resulting mixture was filtered and the filter cake was washed with EtOAc (3×1 mL). The aqueous layer was extracted with EtOAc (3×1 mL). The combined organic layers were dried over anhydrous Na₂SO₄. After filtration, the filtrate was concentrated under reduced pressure. The residue was purified by silica gel column chromatography, eluted with CH₂Cl₂/MeOH (10:1) to afford 1-{6-[2-(methoxymethoxy)-4-(6-methoxypyrimidin-4-yl)phenyl]pyridazin-3-yl}-N-(1-methylcyclobutyl)pyrrolidin-3-amine as a solid.

### Synthesis of Compounds 383 and 384

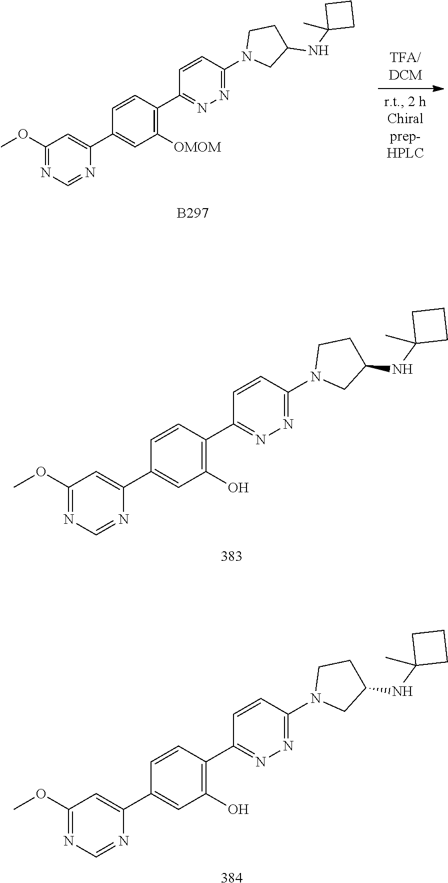

Into a 10 mL vial were added 1-{6-[2-(methoxymethoxy)-4-(6-methoxypyrimidin-4-yl)phenyl]pyridazin-3-yl}-N-(1-methylcyclobutyl)pyrrolidin-3-amine (150 mg, 0.315 mmol, 1 equiv). TFA) and DCM) at room temperature. The solution was stirred for 2 h at room temperature. The resulting mixture was concentrated under reduced pressure. The crude product was purified by Chiral-Prep-HPLC with the following conditions (2 #SHIMADZU (HPLC-01)): Column, Kinetex EVO prep C18, 30*150, 5 um; mobile phase, Water (10 mmol/L NH₄HCO₃) and ACN (20% ACN up to 60% in 8 min) to afford 5-(6-methoxypyrimidin-4-yl)-2-(6-{3-[(1-methylcyclobutyl)amino]pyrrolidin-1-yl}pyridazin-3-yl)phenol (80 mg, 58.77%). The 5-(6-methoxypyrimidin-4-yl)-2-(6-{3-[(1-methylcyclobutyl)amino]pyrrolidin-1-yl}pyridazin-3-yl)phenol (80 mg) was purified by Chiral-Prep-HPLC with the following conditions (2 #SHIMADZU (HPLC-01)): Column, CHIRAL ART Cellulose-SB, 2*25 cm, 5 um; mobile phase, MtBE(0.1% DEA) and MeOH- (hold 30% MeOH- in 8 min) to afford 5-(6-methoxypyrimidin-4-yl)-2-{6-[(3R)-3-[(1-methylcyclobutyl)amino]pyrrolidin-1-yl]pyridazin-3-yl}phenol (21.7 mg, 36.17%) as a light yellow solid.) and 5-(6-methoxypyrimidin-4-yl)-2-{6-[(3S)-3-[(1-methylcyclobutyl)amino]pyrrolidin-1-yl]pyridazin-3-yl}phenol (20.3 mg, 33.83%) as a solid. Compound 383: LCMS:(ES, m/z):432[M+H]^{+ 1}H NMR: (400 MHz, DMSO-d₆) δ 13.91 (s, 1H), 8.86 (d, J=1.0 Hz, 1H), 8.27 (d, J=9.8 Hz, 1H), 8.01 (d, J=8.6 Hz, 1H), 7.74 (d, J=7.6 Hz, 2H), 7.52 (d, J=1.1 Hz, 1H), 7.15 (d, J=9.7 Hz, 1H), 3.98 (s, 3H), 3.74 (dd, J=10.3, 6.6 Hz, 1H), 3.66 (s, 1H), 3.53-3.38 (m, 2H), 3.15 (dd, J=10.4, 6.7 Hz, 1H), 2.15 (tq, J 6.9, 4.3 Hz, 2H), 1.93 (h, J=8.0 Hz, 2H), 1.85-1.73 (m, 3H), 1.72-1.58 (m, 2H), 1.26 (s, 3H). Compound 384: LCMS:(ES, m/z):432[M+H]^{+ 1}H NMR: (400 MHz, DMSO-d₆) δ 13.90 (s, 1H), 8.86 (d, J=1.1 Hz, 1H), 8.27 (d, J=9.8 Hz, 1H), 8.01 (d, J=8.4 Hz, 1H), 7.74 (d, J=7.4 Hz, 2H), 7.52 (d, J=1.1 Hz, 1H), 7.16 (d, J=9.7 Hz, 1H), 3.99 (s, 3H), 3.74 (dd, J=10.4, 6.5 Hz, 1H), 3.67 (s, 1H), 3.47 (ddd, J=18.2, 11.8, 7.3 Hz, 2H), 3.16 (dd, J=10.5, 6.7 Hz, 1H), 2.15 (ddt, J=11.8, 6.9, 4.4 Hz, 2H), 1.94 (q, J=9.5 Hz, 2H), 1.78 (pt, J=8.1, 4.5 Hz, 3H), 1.73-1.57 (m, 2H), 1.26 (s, 3H).

### Example 119: Synthesis of Compounds 399 and 400

### Synthesis of Intermediate 298

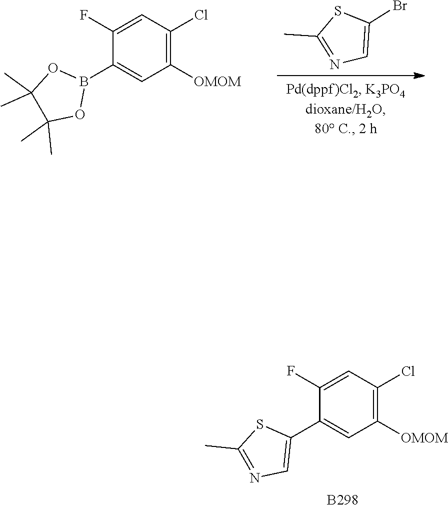

To a stirred solution of 2-[4-chloro-2-fluoro-5-(methoxymethoxy)phenyl]-4,4,5,5-tetramethyl-1,3,2-dioxaborolane (1.9 g, 6.002 mmol, 1 equiv) and 5-bromo-2-methyl-1,3-thiazole (1.07 g, 6.002 mmol, 1 equiv) and K₃PO₄ (3.82 g, 18.006 mmol, 3 equiv) in 1,4-dioxane (5 mL) and H₂O (1 mL) were added Pd(dppf)Cl₂ (0.44 g, 0.600 mmol, 0.1 equiv) dropwise portions at room temperature under nitrogen atmosphere. The resulting mixture was stirred for 2 h at 80° C. under nitrogen atmosphere. The mixture was allowed to cool down to room temperature. The reaction was quenched with H₂O at room temperature. The resulting mixture was extracted with EA (2×10 mL). The combined organic layers were washed with NaCl Solution (1×10 mL), dried over anhydrous Na₂SO₄. After filtration, the filtrate was concentrated under reduced pressure. The residue was purified by silica gel column chromatography, eluted with PE:EA (1:1) to afford 5-[4-chloro-2-fluoro-5-(methoxymethoxy)phenyl]-2-methyl-1,3-thiazole (1 g, 58%) as a solid. LCMS:(ES, m/z):288 [M+H]⁺

### Synthesis of Intermediate B299

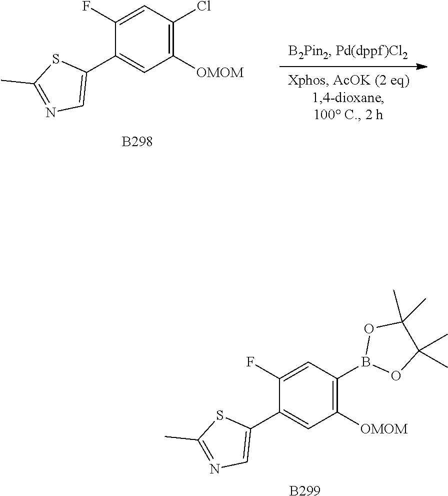

To a stirred solution of 5-[4-chloro-2-fluoro-5-(methoxymethoxy)phenyl]-2-methyl-1,3-thiazole (1 g, 3.475 mmol, 1 equiv) and 4,4,5,5-tetramethyl-2-(tetramethyl-1,3,2-dioxaborolan-2-yl)-1,3,2-dioxaborolane (1.32 g, 5.213 mmol, 1.5 equiv) and AcOK (0.68 g, 6.950 mmol, 2 equiv) in 1,4-dioxane (3 mL) were added XPhos (0.08 g, 0.174 mmol, 0.05 equiv) and Pd(dppf)Cl₂ (0.25 g, 0.348 mmol, 0.1 equiv) dropwise portions at room temperature under nitrogen atmosphere. The resulting mixture was stirred for 2 h at 100° C. under nitrogen atmosphere. The mixture was allowed to cool down to room temperature. The reaction was quenched with H₂O at room temperature. The resulting mixture was extracted with EA (2×5 mL). The combined organic layers were washed with NaCl Solution (1×10 mL), dried over anhydrous Na₂SO₄. After filtration, the filtrate was concentrated under reduced pressure. The residue was purified by silica gel column chromatography, eluted with PE:EA (3:1) to afford 5-[2-fluoro-5-(methoxymethoxy)-4-(4,4,5,5-tetramethyl-1,3,2-dioxaborolan-2-yl)phenyl]-2-methyl-1,3-thiazole (840 mg, 64%) as a solid. LCMS:(ES, m/z):380 [M+H]⁺

### Synthesis of Intermediate B300

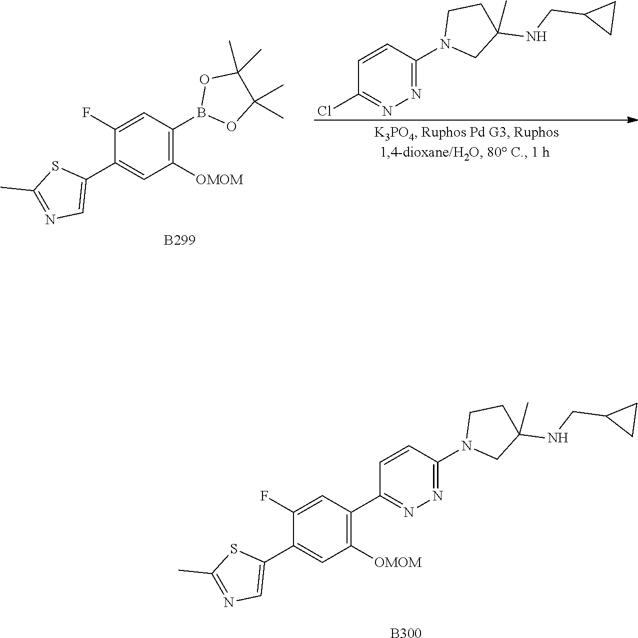

To a stirred solution of 5-[2-fluoro-5-(methoxymethoxy)-4-(4,4,5,5-tetramethyl-1,3,2-dioxaborolan-2-yl)phenyl]-2-methyl-1,3-thiazole (210 mg, 0.554 mmol, 1 equiv) and 1-(6-chloropyridazin-3-yl)-N-(cyclopropylmethyl)-3-methylpyrrolidin-3-amine (147.72 mg, 0.554 mmol, 1 equiv) and K₃PO₄ (352.61 mg, 1.662 mmol, 3 equiv) in 1,4-dioxane (2 mL) and H₂O (0.5 mL) were added RuPhos (25.84 mg, 0.055 mmol, 0.1 equiv) and RuPhos Palladacycle Gen.3 (46.31 mg, 0.055 mmol, 0.1 equiv) dropwise portions at room temperature under nitrogen atmosphere. The resulting mixture was stirred for 1 h at 80° C. under nitrogen atmosphere. The mixture was allowed to cool down to room temperature. The reaction was quenched with H₂O at room temperature. The resulting mixture was extracted with EA (2×5 mL). The combined organic layers were washed with NaCl Solution (1×10 mL), dried over anhydrous Na₂SO₄. After filtration, the filtrate was concentrated under reduced pressure. The residue was purified by silica gel column chromatography, eluted with PE:EA (1:1) to afford N-(cyclopropylmethyl)-1-{6-[5-fluoro-2-(methoxymethoxy)-4-(2-methyl-1,3-thiazol-5-yl)phenyl]pyridazin-3-yl}-3-methylpyrrolidin-3-amine (130 mg, 49%) as a solid. LCMS:(ES, m/z): 484 [M+H]⁺

### Synthesis of Intermediate B301

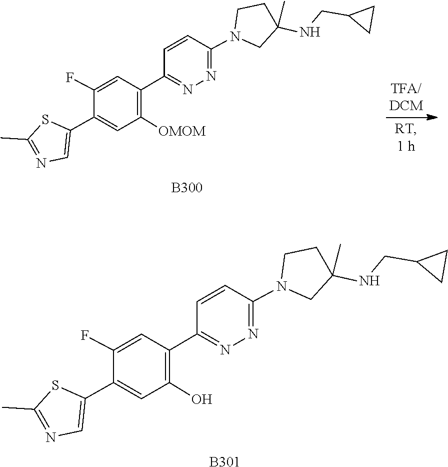

To a stirred solution of N-(cyclopropylmethyl)-1-{6-[5-fluoro-2-(methoxymethoxy)-4-(2-methyl-1,3-thiazol-5-yl)phenyl]pyridazin-3-yl}-3-methylpyrrolidin-3-amine (140 mg, 0.289 mmol, 1 equiv) in DCM (1 mL) was added TFA (1.5 mL) dropwise at room temperature. The mixture was stirred 1 h at room temperature. The mixture was basified to pH 8 with NH₃·H₂O.

The resulting mixture was diluted with H₂O (3 mL). The resulting mixture was extracted with DCM (2×5 mL). The combined organic layers were washed with NaCl Solution (1×10 mL), dried over anhydrous Na₂SO₄. After filtration, the filtrate was concentrated under reduced pressure. This resulted in 2-(6-{3-[(cyclopropylmethyl)amino]-3-methylpyrrolidin-1-yl}pyridazin-3-yl)-4-fluoro-5-(2-methyl-1,3-thiazol-5-yl)phenol (100 mg, 79%) as a solid. LCMS:(ES, m/z): 440 [M+H]⁺

### Synthesis of Compounds 399 and 400

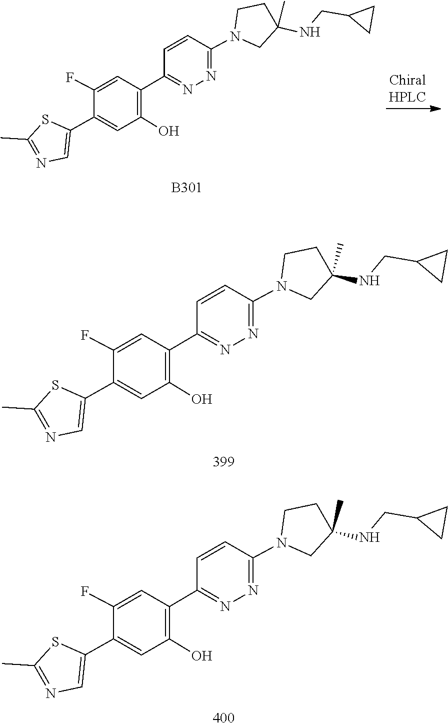 [SMILES: Cc1ncc(-c2cc(O)c(-c3ccc(N4CCC(C)(NCC5CC5)C4)nn3)cc2F)s1 ; "B301" ; Cc1ncc(-c2cc(O)c(-c3ccc(N4CC[C@@](C)(NCC5CC5)C4)nn3)cc2F)s1 ; "399" ; Cc1ncc(-c2cc(O)c(-c3ccc(N4CC[C@](C)(NCC5CC5)C4)nn3)cc2F)s1 ; "400"]

The residue was purified by reverse flash chromatography with the following conditions: Column: CHIRALPAK IH-3, 4.6*50 mm, 3 m; Mobile Phase A: Hex(0.1% DEA): (EtOH:DCM=1:1)=65: 35; Flow rate: 1 mL/min; Gradient: 0% B to 0% B; Injection Volume: Sul mL to afford first eluting 2-{6-[(3R)-3-[(cyclopropylmethyl)amino]-3-methylpyrrolidin-1-yl]pyridazin-3-yl}-4-fluoro-5-(2-methyl-1,3-thiazol-5-yl)phenol (15.1 mg, 14.98%) as a light yellow solid and second eluting 2-{6-[(3S)-3-[(cyclopropylmethyl)amino]-3-methylpyrrolidin-1-yl]pyridazin-3-yl}-4-fluoro-5-(2-methyl-1,3-thiazol-5-yl)phenol (11.6 mg, 12%) as a solid. Compound 399: LCMS:(ES, m/z): 440 [M+H]^{+ 1}H NMR: (400 MHz, DMSO-d₆) δ 13.75 (s, 1H), 8.25 (d, J=9.8 Hz, 1H), 8.16 (s, 1H), 7.93 (d, J=12.7 Hz, 1H), 7.29 (d, J=6.8 Hz, 1H), 7.15 (d, J=9.8 Hz, 1H), 3.63 (s, 1H), 3.56 (s, 1H), 3.48 (d, J=10.7 Hz, 1H), 2.71 (s, 3H), 2.42 (d, J=6.6 Hz, 2H), 2.03 (dt, J=13.0, 6.9 Hz, 1H), 1.83 (dt, J=12.5, 7.1 Hz, 1H), 1.73 (s, 1H), 1.23 (s, 3H), 0.85-0.80 (m, 1H), 0.40-0.39 (m, 2H), 0.14-0.08 (m, 2H). Compound 400: LCMS:(ES, m/z): 440 [M+H]⁺¹H NMR: (400 MHz, DMSO-d₆) δ 13.75 (s, 1H), 8.25 (d, J=9.8 Hz, 1H), 8.16 (s, 1H), 7.93 (d, J=12.7 Hz, 1H), 7.29 (d, J=6.8 Hz, 1H), 7.15 (d, J=9.7 Hz, 1H), 3.63 (s, 1H), 3.56 (s, 1H), 3.48 (d, J=10.7 Hz, 1H), 2.71 (s, 3H), 2.42 (d, J=6.6 Hz, 2H), 2.03 (dt, J=13.0, 7.0 Hz, 1H), 1.83 (dt, J=12.6, 7.2 Hz, 1H), 1.74 (s, 1H), 1.23 (s, 3H), 0.88-0.78 (m, 1H), 0.40-0.34 (m, 2H), 0.15-0.08 (m, 2H).

### Example 120: Synthesis of Compound 403

### Synthesis of Intermediate B302

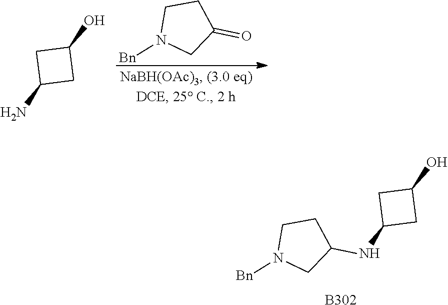

To a stirred solution of 1-benzylpyrrolidin-3-one hydrochloride (1 g, 4.72 mmol, 1.00 equiv) and (1s,3s)-3-aminocyclobutan-1-ol (0.41 g, 4.72 mmol, 1.00 equiv) in DCE (70 mL) was added NaBH(OAc)₃ (3.00 g, 14.17 mmol, 3.00 equiv) and the reaction was reacted for 2 hours at 25° C. under nitrogen atmosphere. The reaction was quenched by the addition of water (3 mL) at 0° C. The reaction mixture was extracted with EtOAc (3×20 mL). The combined organic layers were washed with brine (2×10 mL), dried over anhydrous Na₂SO₄. The reaction mixture was concentrated under reduced pressure. The residue was purified by silica gel column chromatography, eluted with PE/EA (1:1) to afford crude product (1s,3s)-3-[(1-benzylpyrrolidin-3-yl)amino]cyclobutan-1-ol (2.5 g, 68%) as an oil.

### Synthesis of Intermediate B303

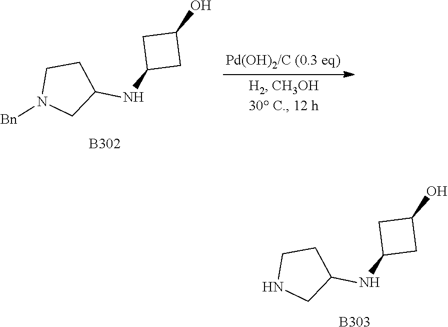

To a stirred solution of (1s,3s)-3-(pyrrolidin-3-ylamino)cyclobutan-1-ol (1.5 g, 6.097 mmol, 1 equiv) in MeOH was added Pd(OH)₂/C (0.4 g) at 30° C. under hydrogen atmosphere and the reaction was reacted for 12 hours at 30° C. under hydrogen atmosphere. The reaction mixture was filtrated and washed with MeOH (3×10 mL). The reaction mixture was concentrated under reduced pressure to afford (1s,3s)-3-[(1-benzylpyrrolidin-3-yl)amino]cyclobutan-1-ol (0.8 g, 84%) as an oil.

### Synthesis of Intermediate B304

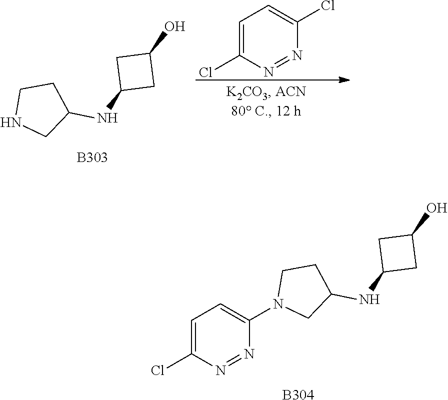

To a stirred solution of (1s,3s)-3-(pyrrolidin-3-ylamino)cyclobutan-1-ol (300 mg, 1.920 mmol, 1 equiv) and pyridazine, 3,6-dichloro- (286.06 mg, 1.920 mmol, 1 equiv) in MeCN (20 mL)was added K₂CO₃ (796.17 mg, 5.760 mmol, 3 equiv) and the reaction was reacted for 12 hat 80° C. under nitrogen atmosphere. The reaction mixture was filtrated and the reaction mixture was concentrated under reduced pressure to afford (1r,3r)-3-{[1-(6-chloropyridazin-3-yl)pyrrolidin-3-yl]amino}cyclobutan-1-ol (300 mg, 58%) as an oil.

### Synthesis of Intermediate B305

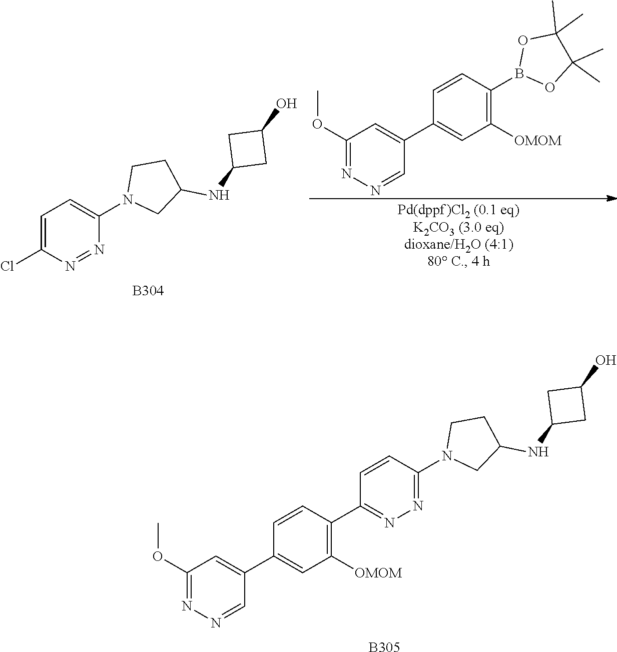

To a stirred solution of (1s,3s)-3-{[1-(6-chloropyridazin-3-yl)pyrrolidin-3-yl]amino}cyclobutan-1-ol (100 mg, 0.372 mmol, 1 equiv) and 3-methoxy-5-[3-(methoxymethoxy)-4-(4,4,5,5-tetramethyl-1,3,2-dioxaborolan-2-yl)phenyl]pyridazine (166.21 mg, 0.446 mmol, 1.2 equiv) in 1,4-dioxane (4 mL) and water (1 mL) was added K₂CO₃ (154.28 mg, 1.116 mmol, 3 equiv), Pd(dppf)Cl₂ (27.23 mg, 0.037 mmol, 0.1 equiv) under nitrogen atmosphere and the reaction mixture was reacted at 80° C. for 4 h. The reaction mixture was concentrated under reduced pressure. The residue was purified by silica gel column chromatography, eluted with CH₂Cl₂/MeOH (10:1) to afford (1s,3s)-3-[(1-{6-[2-(methoxymethoxy)-4-(6-methoxypyridazin-4-yl)phenyl]pyridazin-3-yl}pyrrolidin-3-yl)amino]cyclobutan-1-ol (60 mg, 34%) as an oil.

### Synthesis of Compound 403

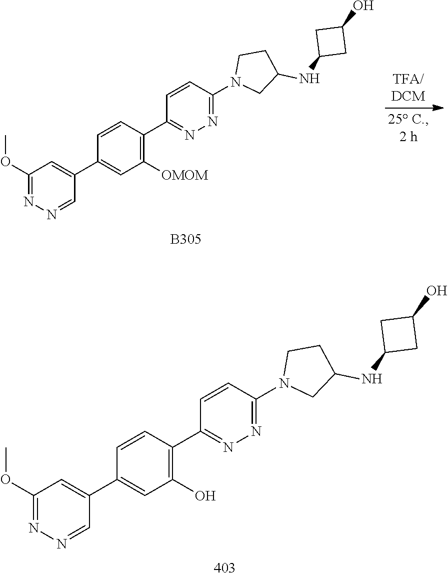

To a solution of (1s,3s)-3-((1-(6-(2-(methoxymethoxy)-4-(6-methoxypyridazin-4-yl)phenyl)pyridazin-3-yl)pyrrolidin-3-yl)amino)cyclobutan-1-ol (60 mg, 0.125 mmol, 1 equiv) in DCM (2 mL) were added TFA (0.5 mL). After stirring for 1 h at 25° C. under a nitrogen atmosphere, the resulting mixture was concentrated under reduced pressure. The crude product was purified by Chiral-Prep-HPLC with the following conditions (2 #SHIMADZU (HPLC-01)): Column, Kinetex EVO prep C18, 30*150, 5 um; mobile phase, Water (10 mmol/L NH₄HCO₃) and ACN (10% ACN up to 48% in 8 min) to afford 5-(6-methoxypyridazin-4-yl)-2-[6-(3-{[(1s,3s)-3-hydroxycyclobutyl]amino}pyrrolidin-1-yl)pyridazin-3-yl]phenol (19.9 mg, 31%) as a solid. LCMS (ES, m/z):434.9 [M+H]⁺¹H NMR (400 MHz, DMSO-d₆) δ 14.11 (s, 1H), 9.35 (d, J=1.9 Hz, 1H), 8.30 (d, J=9.9 Hz, 1H), 8.04 (d, J=8.4 Hz, 1H), 7.55 (d, J=2.0 Hz, 1H), 7.51-7.43 (m, 2H), 7.17 (d, J=9.8 Hz, 1H), 4.90 (d, J=5.9 Hz, 1H), 4.08 (s, 3H), 3.77 (p, J 7.2 Hz, 1H), 3.64 (dd, J=10.8, 5.8 Hz, 2H), 3.50 (d, J=8.8 Hz, 1H), 3.36 (d, J=5.7 Hz, 1H), 3.29 (s, 1H), 2.83-2.62 (m, 1H), 2.50 (q, J=1.9 Hz, 2H), 2.15-2.06 (m, 1H), 2.03 (d, J=12.6 Hz, 1H), 1.83 (dq, J=13.0, 6.6 Hz, 1H), 1.61-1.48 (m, 2H).

### Example 121: Synthesis of Compound 404

### Synthesis of Intermediate B306

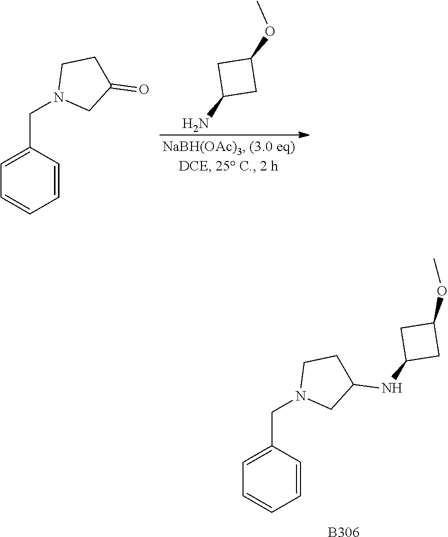

To a stirred solution of 1-benzylpyrrolidin-3-one hydrochloride (1.00 g, 4.724 mmol, 1 equiv) and (1s,3s)-3-methoxycyclobutan-1-amine (0.48 g, 4.724 mmol, 1 equiv) in DCE (70 mL) was added NaBH(OAc)₃ (3.00 g, 14.172 mmol, 3.0 equiv) at 25° C. under nitrogen atmosphere and the reaction was reacted for 2 hours. The reaction was quenched with water (3 mL) at 0° C. The reaction mixture was extracted with DCM (3×20 mL). The combined organic layers were washed with brine (2×10 mL), dried over anhydrous Na₂SO₄. The residue was purified by silica gel column chromatography, eluted with CH₂Cl₂/MeOH (1:1) to afford (1s,3s)-3-[(1-benzylpyrrolidin-3-yl)amino]cyclobutan-1-ol (1 g, 67%) as an oil.

### Synthesis of Intermediate B307

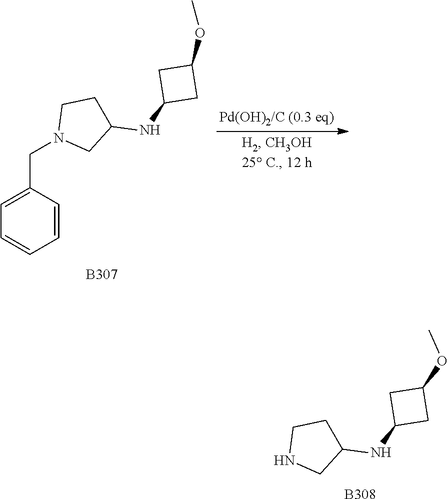

To a stirred solution of N-[(1s,3s)-3-methoxycyclobutyl]pyrrolidin-3-amine (900 mg, 5.286 mmol, 1 equiv) in MeOH (10 mL) was added Pd(OH)₂/C (222 mg) at 25° C. under hydrogen atmosphere and the reaction was reacted for 12 hours. The reaction mixture was filtrated and washed with MeOH (3×10 mL). The reaction mixture was concentrated under reduced pressure to afford 1-benzyl-N-[(1s,3s)-3-methoxycyclobutyl]pyrrolidin-3-amine (300 mg, 51%) as an oil.

### Synthesis of Intermediate B309

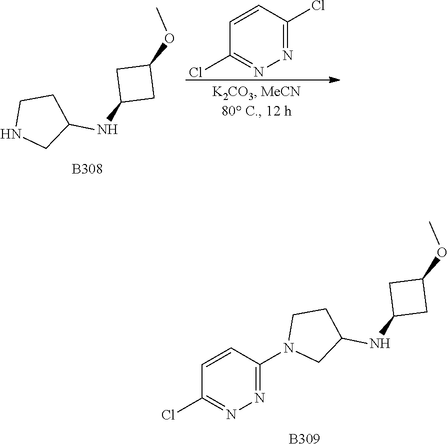

To a stirred solution of N-[(1s,3s)-3-methoxycyclobutyl]pyrrolidin-3-amine (300 mg, 1.762 mmol, 1 equiv) and pyridazine, 3,6-dichloro- (262.25 mg, 1.762 mmol, 1 equiv) in MeCN (5 mL) was added K₂CO₃ (243.5 mg, 3 equiv) and the reaction was reacted at 80° C. under nitrogen atmosphere for 12 h. The reaction mixture was filtrated and the reaction mixture was concentrated under reduced pressure to afford 1-(6-chloropyridazin-3-yl)-N-[(1s,3s)-3-methoxycyclobutyl]pyrrolidin-3-amine (300 mg, 60%) as an oil.

### Synthesis of Intermediate B310

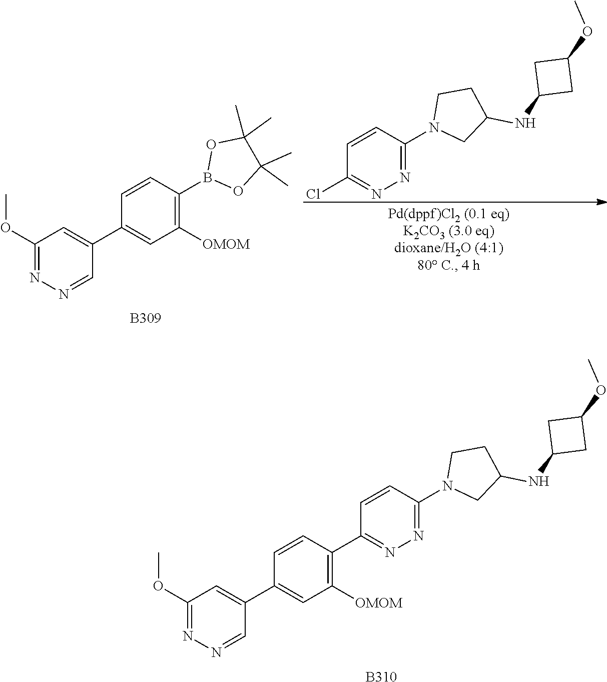

To a stirred solution of 1-(6-chloropyridazin-3-yl)-N-[(1s,3s)-3-methoxycyclobutyl]pyrrolidin-3-amine (100 mg, 0.354 mmol, 1 equiv) and 3-methoxy-5-[3-(methoxymethoxy)-4-(4,4,5,5-tetramethyl-1,3,2-dioxaborolan-2-yl)phenyl]pyridazine (131.64 mg, 0.354 mmol, 1.00 equiv) in 1,4-dioxane(1 mL) and water (0.25 mL) was added K₂CO₃(146.63 mg, 1.06.62 mmol, 3 equiv), Pd(dppf)Cl₂ (25.88 mg, 0.035 mmol, 0.1 equiv) and the reaction was reacted for 4 hours at 80° C. under nitrogen atmosphere. The aqueous layer was extracted with EtOAc (3×10 mL). The residue was purified by silica gel column chromatography, eluted with CH₂Cl₂/MeOH (10:1) to afford 1-{6-[2-(methoxymethoxy)-4-(6-methoxypyridazin-4-yl)phenyl]pyridazin-3-yl}-N-[(1s,3s)-3-methoxycyclobutyl]pyrrolidin-3-amine (80 mg, 46%) as an oil.

### Synthesis of Compound 404

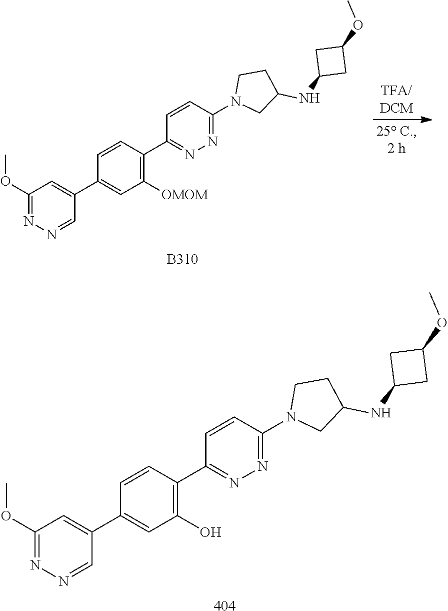

To a solution of N-((1s,3s)-3-methoxycyclobutyl)-1-(6-(2-(methoxymethoxy)-4-(6-methoxypyridazin-4-yl)phenyl)pyridazin-3-yl)pyrrolidin-3-amine (80 mg) in DCM (2 mL) were added TFA (0.5 mL). After stirring for 1 h at 25° C. under a nitrogen atmosphere, the resulting mixture was concentrated under reduced pressure. The crude product was purified by Chiral-Prep-HPLC with the following conditions (2 #SHIMADZU (HPLC-01)): Column, XBridge Shield RP18 OBD Column, 30*150 mm, 5 μm; mobile phase, Water (10 mmol/L NH₄HCO₃) and ACN (10% ACN up to 65% in 10 min) to afford 5-(6-methoxypyridazin-4-yl)-2-[6-(3-{[(1s,3s)-3-methoxycyclobutyl]amino}pyrrolidin-1-yl)pyridazin-3-yl]phenol (18.3 mg, 25%) as a solid.

LCMS (ESI, m/z): 448.9[M+H]⁺¹H NMR (400 MHz, DMSO-d₆) δ 14.07 (s, 1H), 9.35 (d, J=1.9 Hz, 1H), 8.32 (d, J=9.8 Hz, 1H), 8.05 (d, J=8.3 Hz, 1H), 7.55 (d, J=1.9 Hz, 1H), 7.50 (d, J=1.9 Hz, 1H), 7.49-7.46 (m, 1H), 7.20 (d, J=9.7 Hz, 1H), 4.09 (s, 3H), 3.76-3.61 (m, 2H), 3.53-3.43 (m, 3H), 3.32 (s, 1H), 3.12 (s, 3H), 2.95 (s, 1H), 2.62-2.52 (m, 2H), 2.20-2.07 (m, 1H), 1.89 (d, J=6.1 Hz, 1H), 1.64 (s, 2H).

### Example 121: Synthesis of Compound 406

### Synthesis of Intermediate B311

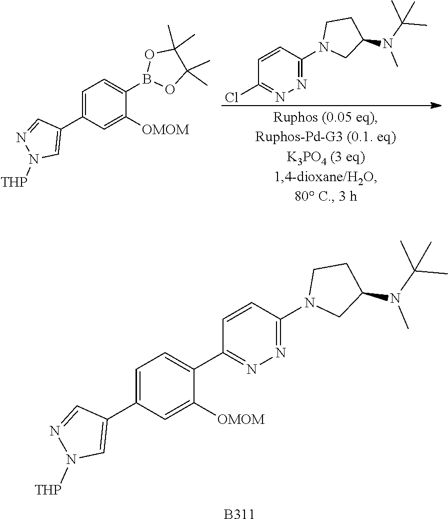

A mixture of 4-[3-(methoxymethoxy)-4-(4,4,5,5-tetramethyl-1,3,2-dioxaborolan-2-yl)phenyl]-1-(oxan-2-yl)pyrazole (101 mg, 0.244 mmol, 1.00 equiv), (3R)—N-tert-butyl-1-(6-chloropyridazin-3-yl)-N-methylpyrrolidin-3-amine (65.53 mg, 0.244 mmol, 1 equiv), (phosphoperoxy)potassium; dipotassium (155.24 mg, 0.732 mmol, 3 equiv), Ruphos (11.38 mg, 0.024 mmol, 0.1 equiv) and RuPhos Palladacycle Gen.3 (20.39 mg, 0.024 mmol, 0.1 equiv) in dioxane (5 mL)/H₂O (1 mL) was added in portions at room temperature under nitrogen atmosphere. The resulting mixture was stirred for additional 3 h at 80° C. After completion of reaction, the mixture was allowed to cool down to room temperature. The reaction was quenched with water (30 mL) and extracted with EtOAc (3×20 mL). The combined organic layers were washed with brine (3×5 mL), dried over anhydrous Na₂SO₄. After filtration, the filtrate was concentrated under reduced pressure. The residue was purified by silica gel column chromatography, eluted with CH₂Cl₂/MeOH (10:3) to afford (3R)—N-tert-butyl-1-{6-[2-(methoxymethoxy)-4-[1-(oxan-2-yl)pyrazol-4-yl]phenyl]pyridazin-3-yl}-N-methylpyrrolidin-3-amine (0.19 g, 150%) as a solid. LCMS (ES, m/z):521[M+H]⁺

### Synthesis of Compound 406

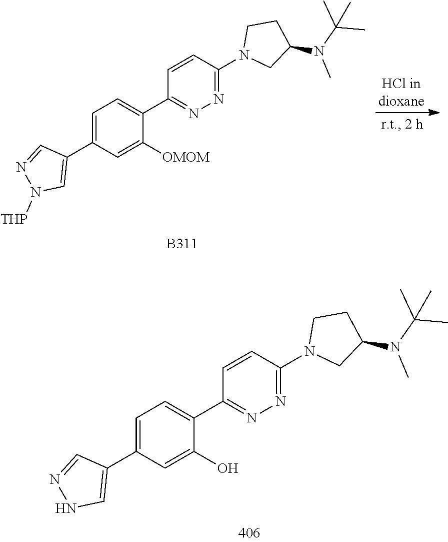

Into an 8 mL sealed tube were added (3R)—N-tert-butyl-1-{6-[2-(methoxymethoxy)-4-[1-(oxan-2-yl)pyrazol-4-yl]phenyl]pyridazin-3-yl}-N-methylpyrrolidin-3-amine (20 mg, 0.038 mmol, 1 equiv) and HCl(gas) in 1,4-dioxane (0.5 mL, 0.380 mmol), the resulting mixture was stirred for 2 h at room temperature. The resulting mixture was concentrated under vacuum. The crude product was purified by Chiral-Prep-HPLC with the following conditions: Column, Kinetex EVO C18 Column, 30×150.5 um; mobile phase, water (10 mmol/L NH₄HCO₃) and ACN (15% ACN up to 55% in 8 min); Detector, UV 220 nm to afford 2-{6-[(3R)-3-[tert-butyl(methyl)amino]pyrrolidin-1-yl]pyridazin-3-yl}-5-(1H-pyrazol-4-yl)phenol (6.6 mg, 4%) as a solid. LCMS:(ES, m/z):393[M+H]^{+ 1}H NMR: (400 MHz, DMSO-d₆) δ 13.90 (s, 1H), 12.96 (s, 1H), 8.20(d, J=12 Hz, 2H), 7.97 (s, 1H), 7.83 (d, J=8.4 Hz, 1H), 7.19-7.16 (m, 3H), 3.97 (s, 1H), 3.70 (t, J=8.8 Hz, 1H), 3.54 (d, J=1.6 Hz, 1H), 3.49 (s, 1H), 3.47-3.36 (m, 2H), 2.23 (s, 3H), 2.08-2.03 (m, 1H), 1.94 (s, 1H), 1.11 (s, 9H) Example 122: Synthesis of Compounds 407 and 408

### Synthesis of Intermediate B312

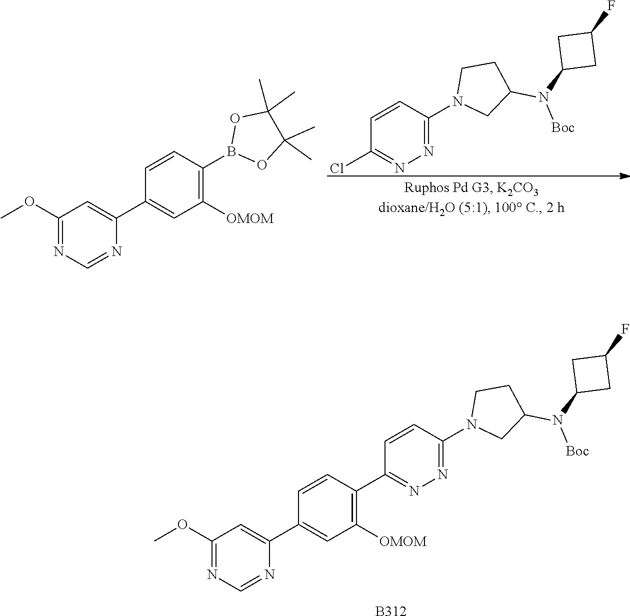

To a solution of 4-methoxy-6-[3-(methoxymethoxy)-4-(4,4,5,5-tetramethyl-1,3,2-dioxaborolan-2-yl) phenyl]pyrimidine (150 mg, 0.403 mmol, 1 equiv) and tert-butyl N-[1-(6-chloropyridazin-3-yl) pyrrolidin-3-yl]-N-[(1s,3s)-3-fluorocyclobutyl]carbamate (224.17 mg, 0.605 mmol, 1.5 equiv) in 1,4-dioxane (2 mL) and H₂O (400 uL) were added K₂CO₃ (139.23 mg, 1.008 mmol, 2.5 equiv) and RuPhos Palladacycle Gen.3 (33.7 mg, 0.040 mmol, 0.1 equiv). After stirring for 2 h at 100° C. under a nitrogen atmosphere, the resulting mixture was concentrated under reduced pressure. The resulting mixture was extracted with EtOAc (3×50 mL). The combined organic layers were washed with brine (3×30 mL), dried over anhydrous Na₂SO₄. After filtration, the filtrate was concentrated under reduced pressure. The residue was purified by Prep-TLC (PE/EA 1:1) to afford tert-butyl N-(1-{6-[2-(methoxymethoxy)-4-(6-methoxypyrimidin-4-yl) phenyl]pyridazin-3-yl}pyrrolidin-3-yl)-N-[(1s,3s)-3-fluorocyclobutyl]carbamate (90 mg, 38%) as a solid.

### Synthesis of Intermediate B313

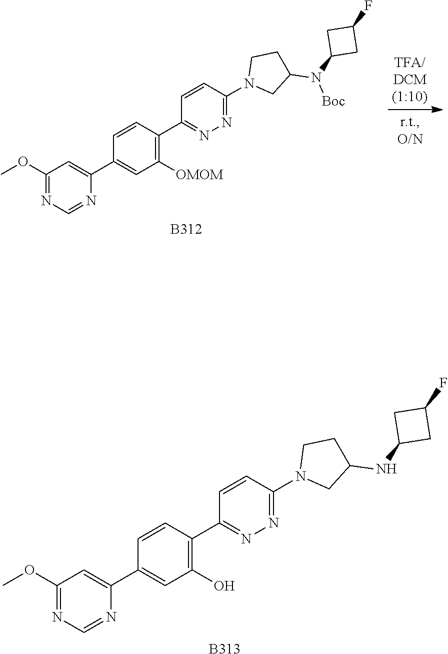

A solution of tert-butyl N-(1-{6-[2-(methoxymethoxy)-4-(6-methoxypyrimidin-4-yl) phenyl]pyridazin-3-yl}pyrrolidin-3-yl)-N-[(1s,3s)-3-fluorocyclobutyl]carbamate (90 mg, 0.155 mmol, 1 equiv) in DCM (1 mL) was treated with TFA (100 uL) for overnight at room temperature. The mixture was basified to pH 8 with saturated Na₂CO₃ (aq.). The resulting mixture was extracted with EtOAc (3×50 mL). The combined organic layers were washed with brine (3×30 mL), dried over anhydrous Na₂SO₄. After filtration, the filtrate was concentrated under reduced pressure. The crude product was purified by Chiral-Prep-HPLC with the following conditions: Column, Kinetex EVO prep C18, 30×150, 5 um; mobile phase, Water (10 mmol/L NH₄HCO₃) and ACN (15% ACN up to 50% in 10 min); Detector, UV 254 nm. This resulted in 5-(6-methoxypyrimidin-4-yl)-2-[6-(3-{[(1s,3s)-3-fluorocyclobutyl]amino}pyrrolidin-1-yl) pyridazin-3-yl]phenol (50 mg, 74%) as a solid.

### Synthesis of compounds 407 and 408

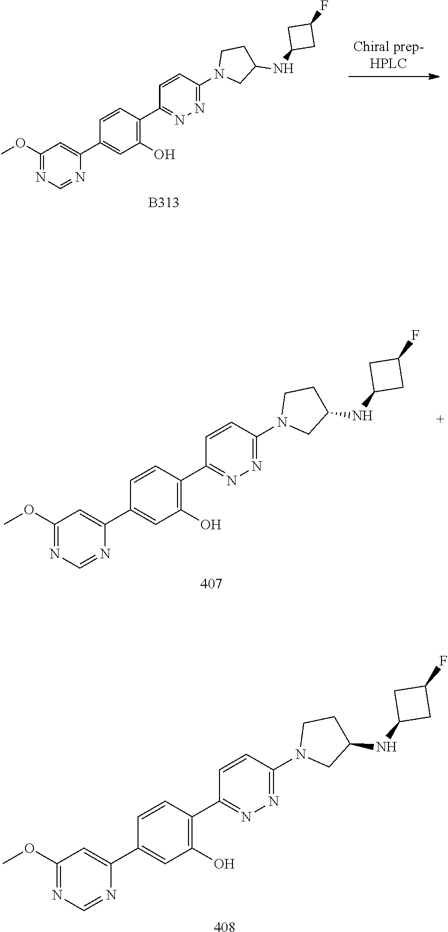 [SMILES: COc1cc(-c2ccc(-c3ccc(N4CCC(N[C@H]5C[C@@H](F)C5)C4)nn3)c(O)c2)ncn1 ; "B313" ; COc1cc(-c2ccc(-c3ccc(N4CC[C@H](N[C@H]5C[C@@H](F)C5)C4)nn3)c(O)c2)ncn1 ; "407" ; COc1cc(-c2ccc(-c3ccc(N4CC[C@@H](N[C@H]5C[C@@H](F)C5)C4)nn3)c(O)c2)ncn1 ; "408"]

5-(6-methoxypyrimidin-4-yl)-2-[6-(3-f{[(1s,3s)-3-fluorocyclobutyl]amino}pyrrolidin-1-yl) pyridazin-3-yl]phenol (50 mg, 0.115 mmol, 1 equiv) was purified by Chiral-Prep-HPLC with the following conditions: Column, CHIRALPAK IA, 2×25 cm, 5 ums; mobile phase, HEX:MtBE=1:1(0.1% DEA) and MeOH- (hold 40% MeOH- in 24 min); Detector, UV 254 nm. This resulted in 5-(6-methoxypyrimidin-4-yl)-2-{6-[(3R)-3-{[(1s,3s)-3-fluorocyclobutyl]amino}pyrrolidin-1-yl]pyridazin-3-yl}phenol (12.2 mg, 24.38%) as a white solid and 5-(6-methoxypyrimidin-4-yl)-2-{6-[(3S)-3-{[(1s,3s)-3-fluorocyclobutyl]amino}pyrrolidin-1-yl]pyridazin-3-yl}phenol (6.5 mg, 13%) as a solid. Compound 407: LCMS: (ES, m/z): 436 [M+H]⁺¹H NMR: (400 MHz, DMSO-d₆) δ 13.91 (s, 1H), 8.87 (d, J=1.0 Hz, 1H), 8.28 (d, J=9.9 Hz, 1H), 8.05-7.98 (m, 1H), 7.78-7.70 (m, 2H), 7.53 (d, J=1.1 Hz, 1H), 7.17 (d, J=9.8 Hz, 1H), 4.78 (dp, J=56.5, 7.0 Hz, 1H), 3.99 (s, 3H), 3.75-3.69 (m, 2H). 3.69-3.59 (m, 1H), 3.51 (d, J=7.7 Hz, 1H), 3.39 (d, J=5.5 Hz, 1H), 2.78 (p, J=7.6 Hz, 1H), 2.71-2.59 (m, 2H), 2.10 (dq, J=12.7, 6.4 Hz, 1H), 1.96-1.80 (m, 3H). Compound 408: LCMS: (ES, m/z): 436 [M+H]⁺¹H NMR: (400 MHz, DMSO-d₆) δ 13.91 (s, 1H), 8.87 (d, J=1.0 Hz, 1H), 8.28 (d, J=9.9 Hz, 1H), 8.05-7.98 (m, 1H), 7.78-7.70 (m, 2H), 7.53 (d, J=1.1 Hz, 1H), 7.17 (d, J=9.8 Hz, 1H), 4.78 (dp, J=56.5, 7.0 Hz, 1H), 3.99 (s, 3H), 3.75-3.69 (m, 2H). 3.69-3.59 (m, 1H), 3.51 (d, J=7.7 Hz, 1H), 3.39 (d, J=5.5 Hz, 1H), 2.78 (p, J=7.6 Hz, 1H), 2.71-2.59 (m, 2H), 2.10 (dq, J=12.7, 6.4 Hz, 1H), 1.96-1.80 (m, 3H).

### Example 123: Synthesis of Compound 420

### Synthesis of Intermediate B314

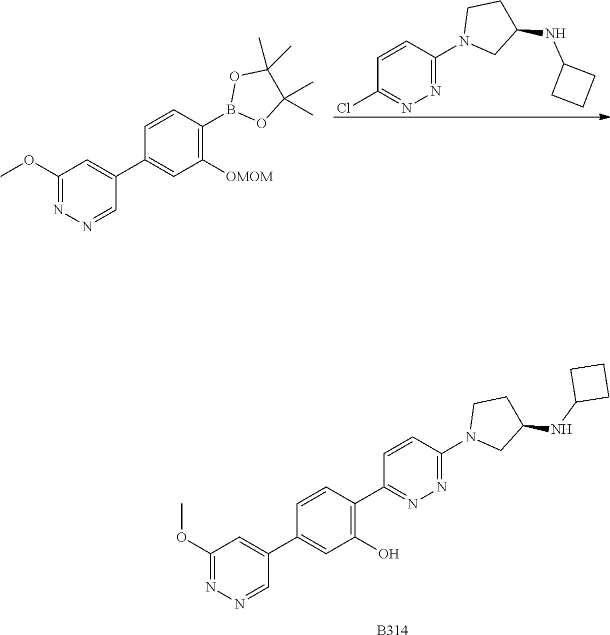

A solution of 3-methoxy-5-[3-(methoxymethoxy)-4-(4,4,5,5-tetramethyl-1,3,2-dioxaborolan-2-yl)phenyl]pyridazine (100 mg, 0.269 mmol, 1 equiv) in 1,4-dioxane (3 mL)/H₂O (0.6 mL) was treated with (3R)-1-(6-chloropyridazin-3-yl)-N-cyclobutylpyrrolidin-3-amine (67.9 mg, 0.269 mmol, 1 equiv), K₃PO₄ (171.08 mg, 0.807 mmol, 3 equiv) and RuPhos Palladacycle Gen.3 (22.47 mg, 0.027 mmol, 0.1 equiv) for 2 h at 100° C. under nitrogen atmosphere. The residue was purified by Prep-TLC (PE/EA 1:3) to afford (3R)—N-cyclobutyl-1-{6-[2-(methoxymethoxy)-4-(6-methoxypyridazin-4-yl)phenyl]pyridazin-3-yl}pyrrolidin-3-amine (80 mg, 64%) as a solid.

LCMS:(ES, m/z):419 [M+H]⁺

### Synthesis of Compound 419

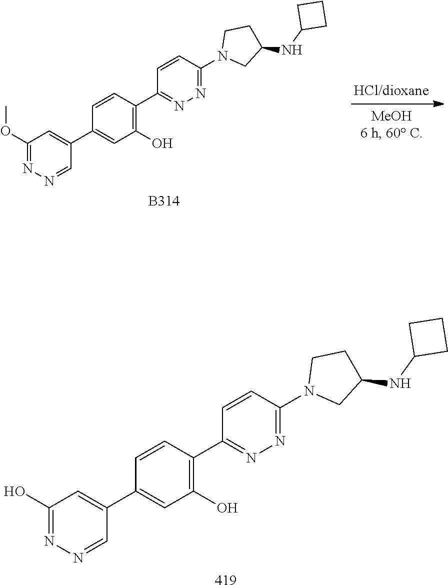 [SMILES: COc1cc(-c2ccc(-c3ccc(N4CC[C@@H](NC5CCC5)C4)nn3)c(O)c2)cnn1 ; "B314" ; Oc1cc(-c2ccc(-c3ccc(N4CC[C@@H](NC5CCC5)C4)nn3)c(O)c2)cnn1 ; "419"]

Into a 5 mL vial were added 2-{6-[(3R)-3-(cyclobutylamino)pyrrolidin-1-yl]pyridazin-3-yl}-5-(6-methoxypyridazin-4-yl)phenol (10 mg, 0.024 mmol, 1 equiv), HCl(gas) in 1,4-dioxane (0.1 mL, 3.291 mmol, 137.74 equiv) and methanol (0.4 mL) at room temperature. The final reaction mixture was stirred for 6 h at 60° C. The resulting mixture was concentrated under reduced pressure. The crude product was purified by Chiral-Prep-HPLC with the following conditions (2 #SHIMADZU (HPLC-01)): Column, Kinetex EVO C18 Column, 30*150.5 um; mobile phase, Water (10 mmol/L NH₄HCO₃) and ACN (10% ACN up to 50% in 10 min) to afford 5-(4-{6-[(3R)-3-(cyclobutylamino)pyrrolidin-1-yl]pyridazin-3-yl}-3-hydroxyphenyl)pyridazin-3-ol (13.9 mg, 23%) as a solid. LCMS:(ES, m/z):405[M+H]^{+ 1}HNMR:(ES, m/z): (400 MHz, DMSO-d₆) δ 14.09 (s, 1H), 13.11 (s, 1H), 8.36-8.26 (m, 2H), 8.02 (d, J=8.2 Hz, 1H), 7.38 (d, J=7.9 Hz, 2H), 7.20-7.15 (m, 2H), 3.68-3.58 (m, 2H), 3.55-3.46 (m, 1H), 3.40 (q, J=5.6 Hz, 1H), 3.27 (dd, J=17.3, 9.7 Hz, 2H), 2.21-2.03 (m, 3H), 1.83 (dq, J=13.0, 6.7 Hz, 1H), 1.76-1.64 (m, 2H), 1.64-1.45 (m, 2H).

### Synthesis of Intermediate B315

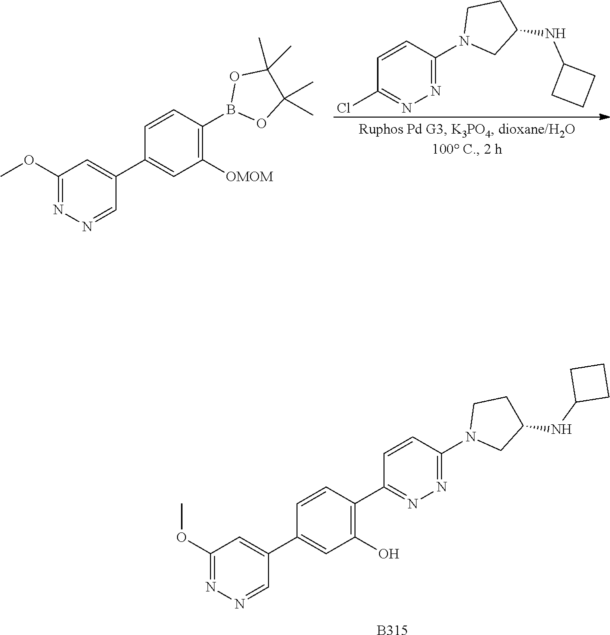

A solution of 3-methoxy-5-[3-(methoxymethoxy)-4-(4,4,5,5-tetramethyl-1,3,2-dioxaborolan-2-yl)phenyl]pyridazine (100 mg, 0.269 mmol, 1 equiv) in 1,4-dioxane (3 mL)/H₂O (0.6 mL) was treated with (3S)-1-(6-chloropyridazin-3-yl)-N-cyclobutylpyrrolidin-3-amine (67.9 mg, 0.269 mmol, 1 equiv), K₃PO₄ (171.08 mg, 0.807 mmol, 3 equiv) and RuPhos Palladacycle Gen.3 (22.47 mg, 0.027 mmol, 0.1 equiv) for 2 h at 100° C. under nitrogen atmosphere. The residue was purified by Prep-TLC (PE/EA 1:3) to afford 2-{6-[(3S)-3-(cyclobutylamino)pyrrolidin-1-yl]pyridazin-3-yl}-5-(6-methoxypyridazin-4-yl)phenol (90 mg, 80%) as a solid. LCMS:(ES, m/z): 419 [M+H]⁺

### Synthesis of Compound 420

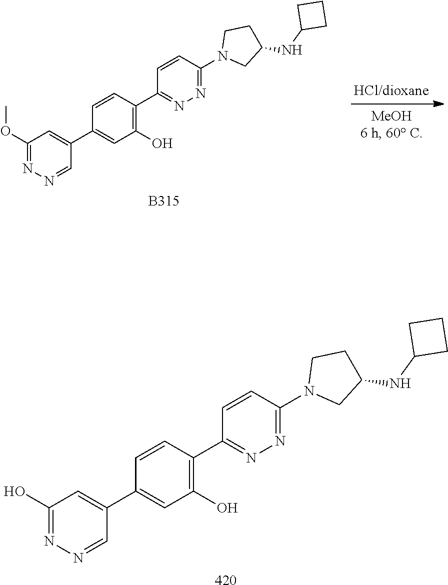 [SMILES: COc1cc(-c2ccc(-c3ccc(N4CC[C@H](NC5CCC5)C4)nn3)c(O)c2)cnn1 ; "B315" ; Oc1cc(-c2ccc(-c3ccc(N4CC[C@H](NC5CCC5)C4)nn3)c(O)c2)cnn1 ; "420"]

Into a 5 mL vial were added 2-{6-[(3S)-3-(cyclobutylamino)pyrrolidin-1-yl]pyridazin-3-yl}-5-(6-methoxypyridazin-4-yl)phenol (90 mg, 0.215 mmol, 1 equiv), HCl(gas) in 1,4-dioxane (0.5 mL) and methanol (2 mL) at room temperature. The final reaction mixture was stirred for 6 h at 60° C. The resulting mixture was concentrated under reduced pressure. The crude product was purified by Chiral-Prep-HPLC with the following conditions (2 #SHIMADZU (HPLC-01)): Column, Kinetex EVO C18 Column, 30*150.5 um; mobile phase, Water (10 mmol/L NH₄HCO₃) and ACN (10% ACN up to 50% in 10 min) to afford 5-(4-{6-[(3S)-3-(cyclobutylamino)pyrrolidin-1-yl]pyridazin-3-yl}-3-hydroxyphenyl)pyridazin-3-ol (20.3 mg, 22.85%) as a solid. LCMS:(ES, m/z): 405 [M+H]^{+ 1}H NMR:(ES, m/z): (400 MHz, DMSO-d₆) δ 14.09 (s, 1H), 13.11 (s, 1H), 8.36-8.26 (m, 2H), 8.02 (d, J=8.1 Hz, 1H), 7.38 (d, J=7.9 Hz, 2H), 7.20-7.14 (m, 2H), 3.70-3.57 (m, 2H), 3.55-3.44 (m, 1H), 3.41-3.36 (m, 1H), 3.25 (p, J=7.6 Hz, 2H), 2.13 (ddd, J=18.1, 10.9, 6.6 Hz, 3H), 1.83 (dq, J=13.1, 6.7 Hz, 1H), 1.76-1.63 (m, 2H), 1.63-1.45 (m, 2H).

### Example 124: Synthesis of Compound 436

### Synthesis of Intermediate B316

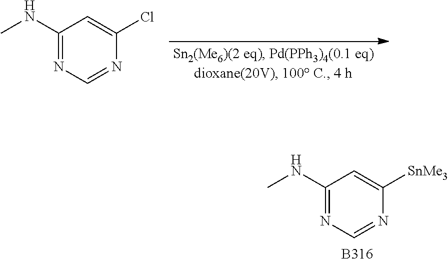

A solution of 6-chloro-N-methylpyrimidin-4-amine (2.7 g, 18.806 mmol, 1 equiv) and Sn₂Me₆ (12.32 g, 37.612 mmol, 2 equiv), Pd(PPh₃)₄(2.17 g, 1.881 mmol, 0.1 equiv) in 1,4-dioxane (54 mL) was stirred for 4 h at 100° C. under nitrogen atmosphere. The mixture was allowed to cool down to room temperature. The resulting mixture was poured into KF(aq). The resulting mixture was extracted with EA (1×150 mL). dried over anhydrous Na₂SO₄. After filtration, the filtrate was concentrated under reduced pressure. This resulted in N-methyl-6-(trimethylstannyl)pyrimidin-4-amine (5.2 g, 101%) as a solid. LCMS:(ES, m/z):274

### Synthesis of Intermediate B317

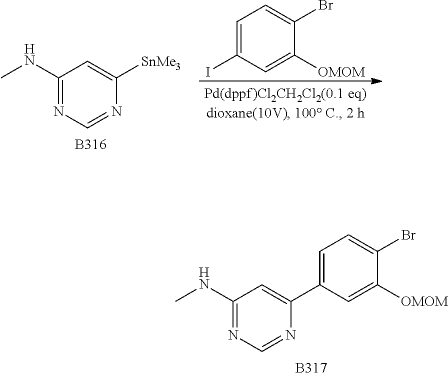

To a solution of 1-bromo-4-iodo-2-(methoxymethoxy)benzene (1.35 g, 3.936 mmol, 1 equiv) and N-methyl-6-(trimethylstannyl)pyrimidin-4-amine (3.21 g, 11.808 mmol, 3 equiv) in 1,4-dioxane (13.5 mL) were added Pd(dppf)Cl₂·CH₂Cl₂ (0.32 g, 0.394 mmol, 0.1 equiv). After stirring for 2 h at 100° C. under a nitrogen atmosphere, the resulting mixture was concentrated under reduced pressure. The residue was purified by Prep-TLC gel column chromatography, eluted with PE/EA (1:1) to afford 6-[4-bromo-3-(methoxymethoxy)phenyl]-N-methylpyrimidin-4-amine (640 mg, 50%) as a solid. LCMS:(ES, m/z):324

### Synthesis of Intermediate B318

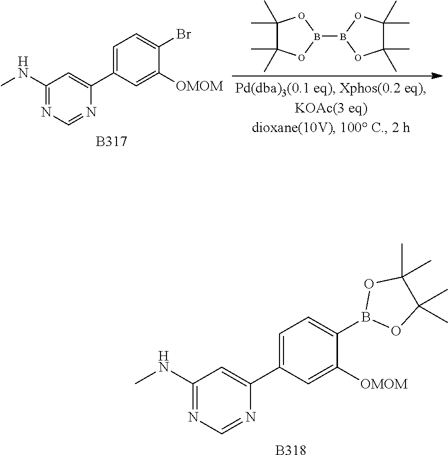

A solution of 6-[4-bromo-3-(methoxymethoxy)phenyl]-N-methylpyrimidin-4-amine (580 mg, 1.789 mmol, 1 equiv) and bis(pinacolato)diboron (682 mg, 2.683 mmol, 1.5 equiv), XPhos (171 mg, 0.358 mmol, 0.2 equiv), KOAC (527 mg, 5.367 mmol, 3 equiv), Pd₂(dba)₃ (164 mg, 0.179 mmol, 0.1 equiv) in 1,4-dioxane (5.8 mL) was stirred for 2 h at 100° C. under nitrogen atmosphere. The mixture was allowed to cool down to room temperature. The resulting mixture was extracted with EA (1×20 mL). dried over anhydrous Na₂SO₄. After filtration, the filtrate was concentrated under reduced pressure. This resulted in 6-[3-(methoxymethoxy)-4-(4,4,5,5-tetramethyl-1,3,2-dioxaborolan-2-yl)phenyl]-N-methylpyrimidin-4-amine (1.2 g, 181%) as a oil. LCMS:(ES, m/z):372

### Synthesis of Intermediate B319

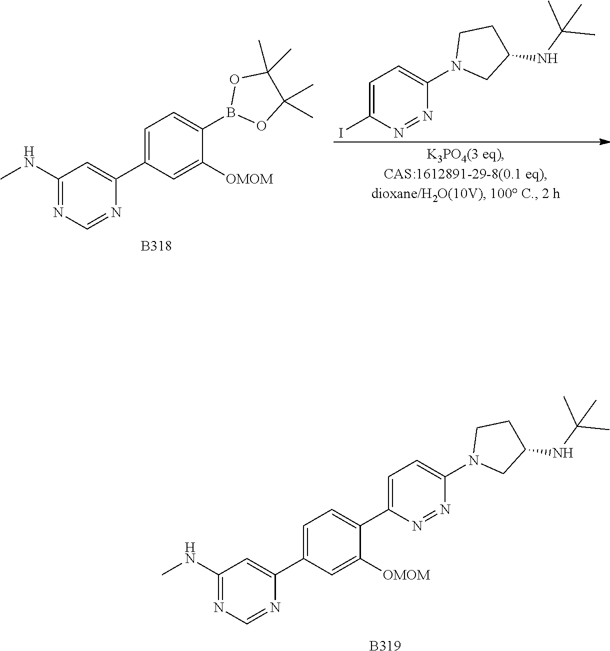

To a solution of (3S)—N-tert-butyl-1-(6-iodopyridazin-3-yl)pyrrolidin-3-amine (100 mg, 0.289 mmol, 1 equiv) and 6-[3-(methoxymethoxy)-4-(4,4,5,5-tetramethyl-1,3,2-dioxaborolan-2-yl)phenyl]-N-methylpyrimidin-4-amine (322 mg, 0.867 mmol, 3 equiv) in 1,4-dioxane (0.8 mL) and water (0.2 mL) were added K₃PO₄ (184 mg, 0.867 mmol, 3 equiv) and {1,3-bis[2,6-bis(pentan-3-yl)phenyl]-4,5-dichloro-2,3-dihydro-1H-imidazol-2-yl}dichloro(2-methyl-llambda4-pyridin-1-yl)palladium (24 mg, 0.029 mmol, 0.1 equiv). After stirring for 2 h at 100° C. under a nitrogen atmosphere, the resulting mixture was concentrated under reduced pressure. The residue was silica gel column chromatography, eluted with CH₂Cl₂/MeOH (10:1) to afford 6-(4-{6-[(3S)-3-(tert-butylamino)pyrrolidin-1-yl]pyridazin-3-yl}-3-(methoxymethoxy)phenyl)-N-methylpyrimidin-4-amine (60 mg, 45%) as a solid. LCMS:(ES, m/z):464

### Synthesis of Compound 436

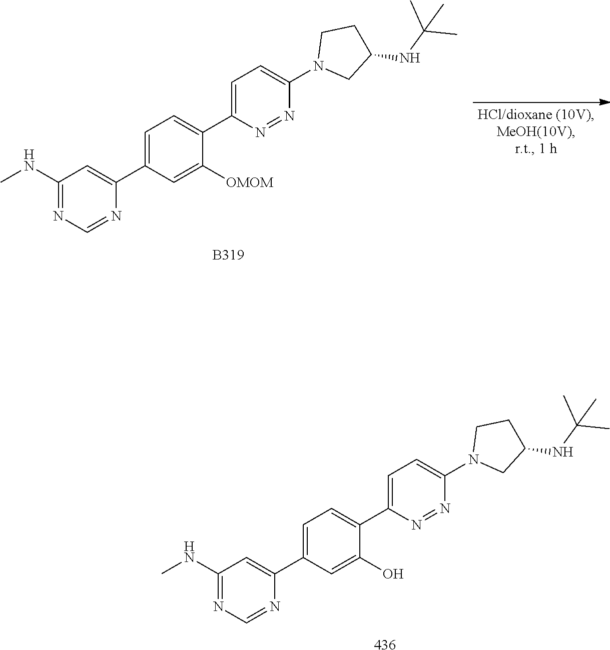

A solution of 6-(4-{6-[(3S)-3-(tert-butylamino)pyrrolidin-1-yl]pyridazin-3-yl}-3-(methoxymethoxy)phenyl)-N-methylpyrimidin-4-amine (54 mg, 0.116 mmol, 1 equiv) in MeOH (1 mL), HCl(gas) in 1,4-dioxane (1 mL) was stirred for 1 h at room temperature. The resulting mixture was concentrated under reduced pressure. The crude product was purified by Prep-HPLC with the following conditions (Column: Kinetex EVO C18 Column, 30×150, 5 um; Mobile Phase A: Water (10 mmol/L NH₄HCO₃), Mobile Phase B: ACN; Flow rate: 60 mL/min; Gradient: 10% B to 45% B in 10 min, 45% B; Wave Length: UV 220 nm; RT1(min): 8.83) to afford 2-{6-[(3S)-3-(tert-butylamino)pyrrolidin-1-yl]pyridazin-3-yl}-5-[6-(methylamino)pyrimidin-4-yl]phenol (3.3 mg, 7%) as a solid. LCMS:(ES, m/z):420 ¹H NMR: (400 MHz, DMSO-d₆) δ 13.81 (s, 1H), 8.50 (s, 1H), 8.24 (d, J=9.8 Hz, 1H), 7.98 (d, J=8.8 Hz, 1H), 7.57 (s, 2H), 7.34 (s, 1H), 7.16 (d, J=9.7 Hz, 1H), 6.94 (d, J=1.2 Hz, 1H), 3.80 (s, 1H), 3.66 (s, 1H), 3.54 (s, 1H), 3.45 (d, J=9.1 Hz, 1H), 3.09 (s, 1H), 2.87 (d, J=4.8 Hz, 3H), 2.19 (s, 1H), 1.77 (s, 1H), 1.09 (s, 9H).

### Example 125: Synthesis of Compound 437

### Synthesis of Intermediate B320

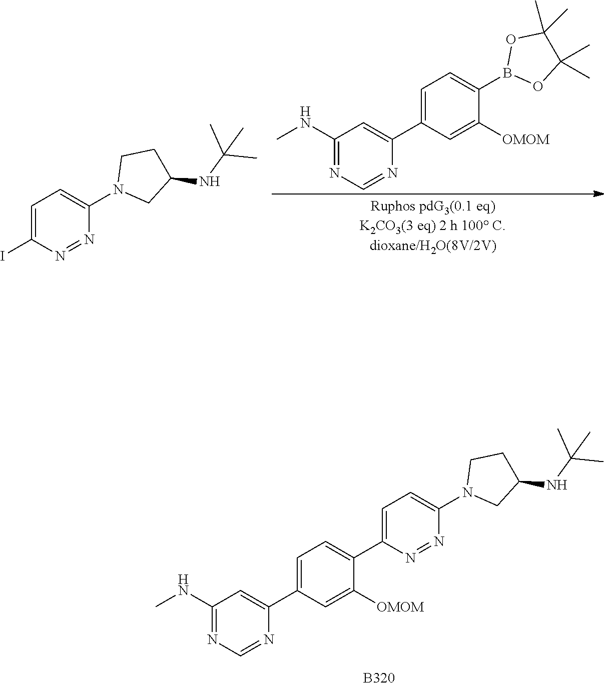

A mixture of (3R)—N-tert-butyl-1-(6-iodopyridazin-3-yl)pyrrolidin-3-amine (100 mg, 0.289 mmol, 1 equiv) and 6-[3-(methoxymethoxy)-4-(4,4,5,5-tetramethyl-1,3,2-dioxaborolan-2-yl)phenyl]-N-methylpyrimidin-4-amine (428 mg, 1.156 mmol, 4 equiv), RuPhos Palladacycle Gen.3 (3.5 mg, 0.433 mmol, 0.1 equiv), K₂CO₃ (119 mg, 0.867 mmol, 3 equiv) in 1,4-dioxane (1 mL), H₂O (0.25 mL) was stirred for 2 h at 100° C. The mixture was allowed to cool down to room temperature. The resulting mixture was filtered and the filter cake was washed with DCM (5×5 mL). The filtrate was concentrated under reduced pressure. The residue was purified by silica gel column chromatography, eluted with CH₂Cl₂/MeOH (10:1) to afford 6-(4-{6-[(3R)-3-(tert-butylamino)pyrrolidin-1-yl]pyridazin-3-yl}-3-(methoxymethoxy)phenyl)-N-methylpyrimidin-4-amine (73 mg, 55%) as an oil. LCMS:(ES, m/z):464[M+H]⁺

### Synthesis of Compound 437

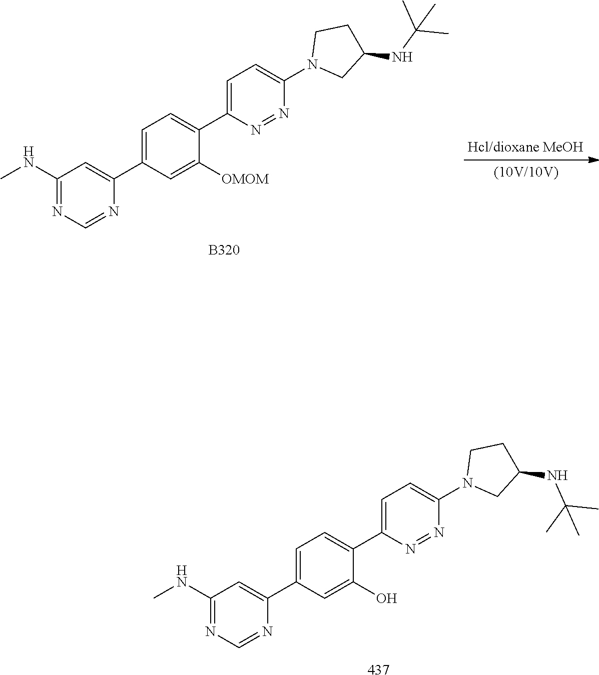

Into a 50 mL 3-necked round-bottom flask were added 6-(4-{6-[(3R)-3-(tert-butylamino)pyrrolidin-1-yl]pyridazin-3-yl}-3-(methoxymethoxy)phenyl)-N-methylpyrimidin-4-amine (68 mg, 0.147 mmol, 1 equiv) and HCl(gas)(0.68 mL) in Meoh (0.68 mL, 22.381 mmol,) at room temperature for 2h. The resulting mixture was concentrated under reduced pressure. The crude product was purified by Prep-HPLC with the following conditions (Column: Kinetex EVO C18 Column, 30*150, 5 um; Mobile Phase A: Water (10 mmol/L NH₄HCO₃), Mobile Phase B: ACN; Flow rate: 60 mL/min; Gradient: 10% B to 45% B in 8 min, 45% B; Wave Length: UV 220 nm; RT1(min): 6.6) to afford 2-{6-[(3R)-3-(tert-butylamino)pyrrolidin-1-yl]pyridazin-3-yl}-5-[6-(methylamino)pyrimidin-4-yl]phenol (2.9 mg, 4%) as a solid. LCMS:(ES, m/z):420[M+H]¹H NMR: (400 MHz, DMSO-d₆) δ 8.50 (s, 1H), 8.24 (d, J=9.8 Hz, 1H), 7.97 (dd, J=8.9, 2.0 Hz, 1H), 7.58 (s, 2H), 7.37 (s, 1H), 7.16 (d, J=9.7 Hz, 1H), 6.95 (s, 1H), 3.85-3.76 (m, 1H), 3.66 (s, 1H), 3.60-3.52 (m, 1H), 3.12 (t, J=8.8 Hz, 1H), 2.87 (d, J=4.6 Hz, 3H), 2.21 (d, J=10.6 Hz, 1H), 1.86-1.72 (m, 1H), 1.10 (s, 9H).

### Example 126: Synthesis of Compound 438 and 439

### Synthesis of Intermediate B321

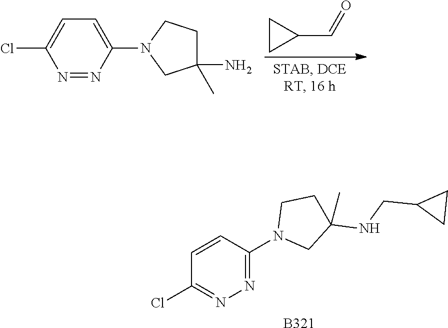

A solution of 1-(6-chloropyridazin-3-yl)-3-methylpyrrolidin-3-amine (800 mg, 3.762 mmol, 1 equiv) in DCE (10 mL) was treated with cyclopropanecarbaldehyde (263.65 mg, 3.762 mmol, 1 equiv) for 2 h at room temperature under nitrogen atmosphere followed by the addition of STAB (1.59 g, 7.524 mmol, 2 equiv) in portions at room temperature. The solution was stirred for 14 h at room temperature under nitrogen atmosphere. The resulting mixture was concentrated under reduced pressure. The mixture was neutralized to pH 9 with saturated NaHCO₃ (aq.). The aqueous layer was extracted with CH₂Cl₂ (3×30 mL). The combined organic layers were dried over anhydrous Na₂SO₄. After filtration, the filtrate was concentrated under reduced pressure. The residue was purified by silica gel column chromatography, eluted with PE/EA (1:1) to afford 1-(6-chloropyridazin-3-yl)-N-(cyclopropylmethyl)-3-methylpyrrolidin-3-amine (400 mg, 40%) as an oil. LCMS:(ES, m/z): 267 [M+H]⁺

### Synthesis of Intermediate B322

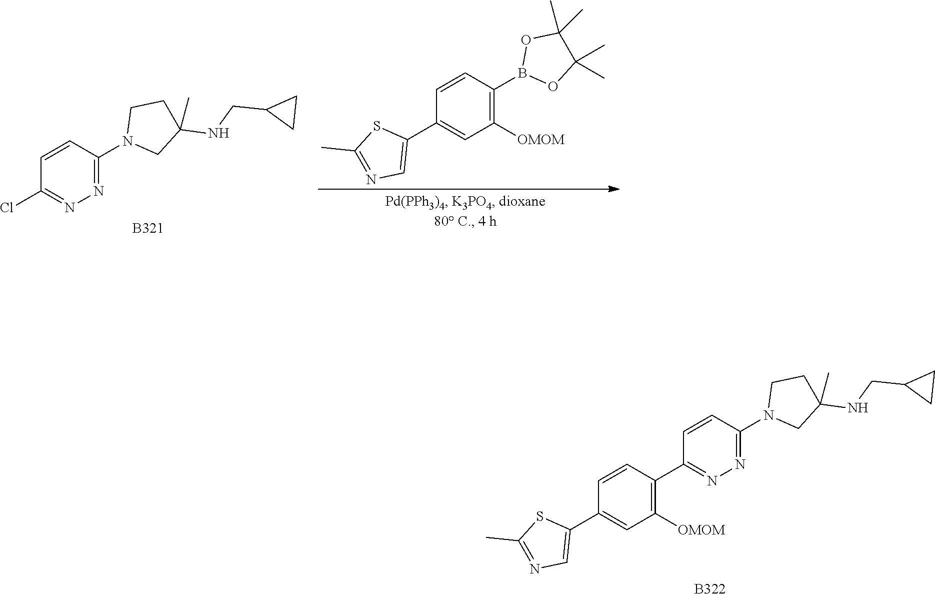

A mixture of 1-(6-chloropyridazin-3-yl)-N-(cyclopropylmethyl)-3-methylpyrrolidin-3-amine (265.84 mg, 0.996 mmol, 1.20 equiv) and 5-[3-(methoxymethoxy)-4-(4,4,5,5-tetramethyl-1,3,2-dioxaborolan-2-yl)phenyl]-2-methyl-1,3-thiazole (300 mg, 0.830 mmol, 1.00 equiv) and K₃PO₄ (528.81 mg, 2.490 mmol, 3 equiv) and Pd(PPh₃)₄(95.96 mg, 0.083 mmol, 0.1 equiv) in dioxane/H₂O(12 mL)(5:1) was stirred for 4 h at 80° C. under nitrogen atmosphere. The resulting liquid was dried over anhydrous Na₂SO₄. After filtration, the filtrate was concentrated under reduced pressure. The residue was purified by silica gel column chromatography, eluted with CH₂Cl₂/MeOH (10:1) to afford N-(cyclopropylmethyl)-1-{6-[2-(methoxymethoxy)-4-(2-methyl-1,3-thiazol-5-yl)phenyl]pyridazin-3-yl}-3-methylpyrrolidin-3-amine (360 mg, 93%) as an oil. LCMS:(ES, m/z): 466 [M+H]⁺

### Synthesis of Intermediate B323

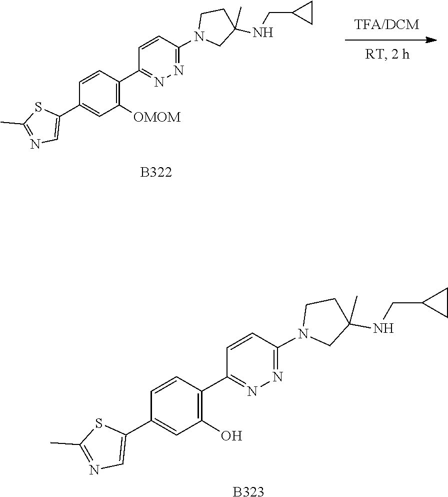

A mixture of N-(cyclopropylmethyl)-1-{6-[2-(methoxymethoxy)-4-(2-methyl-1,3-thiazol-5-yl)phenyl]pyridazin-3-yl}-3-methylpyrrolidin-3-amine (400 mg, 0.859 mmol, 1 equiv) and TFA (2.0 mL) in CH₂Cl₂ (4.0 mL) was stirred for 2 h at room temperature under air atmosphere. The resulting mixture was concentrated under reduced pressure. The residue was neutralized to pH 9 with saturated NaHCO₃ (aq.). The aqueous layer was extracted with CH₂Cl₂ (3×50 mL). This resulted in 2-(6-{3-[(cyclopropylmethyl)amino]-3-methylpyrrolidin-1-yl}pyridazin-3-yl)-5-(2-methyl-1,3-thiazol-5-yl)phenol (207 mg, 52%) as a solid.

### Synthesis of Compounds 438 and 439

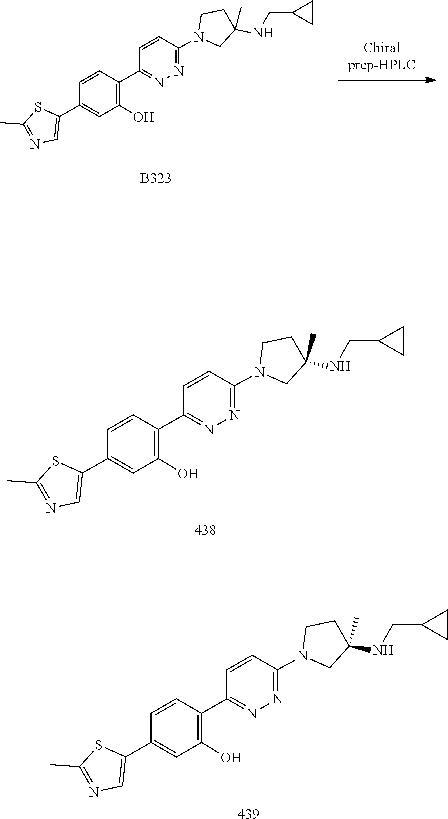 [SMILES: Cc1ncc(-c2ccc(-c3ccc(N4CCC(C)(NCC5CC5)C4)nn3)c(O)c2)s1 ; "B323" ; Cc1ncc(-c2ccc(-c3ccc(N4CC[C@](C)(NCC5CC5)C4)nn3)c(O)c2)s1 ; "438" ; Cc1ncc(-c2ccc(-c3ccc(N4CC[C@@](C)(NCC5CC5)C4)nn3)c(O)c2)s1 ; "439"]

The crude product was purified by Chiral-Prep-HPLC with the following conditions (2SHIMADZU (HPLC-01): Column: CHIRALPAK IF, 3*25 cm, 5 m; Mobile Phase A: MtBE(0.1% DEA)-HPLC, Mobile Phase B: IPA-HPLC; Flow rate: 20 mL/min; Gradient: 20% B to 20% B in 17 min; Wave Length: UV 254/220 nm; RT1(min): 12.7; RT2(min): 14.8; Sample Solvent: MeOH-HPLC; Injection Volume: 0.35 mL; Number Of Runs: 13) to afford Compound 438 (62.6 mg, 16%) as a solid and Compound 439 (64.9 mg, 16.46%) as a solid. LCMS (ES, m/z): 422 [M+H]⁺Compound 438: 1H NMR (400 MHz, DMSO-d₆) δ 14.03 (s, 1H), 8.23 (d, J=9.8 Hz, 1H), 8.11 (s, 1H), 7.92 (d, J=8.8 Hz, 1H), 7.21-7.12 (m, 3H), 3.63 (t, J=7.4 Hz, 1H), 3.60-3.53 (m, 1H), 3.49 (d, J=10.4 Hz, 1H), 3.36 (s, 1H), 2.69 (s, 3H), 2.44 (d, J=5.9 Hz, 2H), 2.04 (s, 1H), 1.85 (s, 1H), 1.24 (s, 3H), 0.85 (s, 1H), 0.44-0.35 (m, 2H), 0.12 (d, J=4.9 Hz, 2H). Compound 439: LCMS (ES, m/z): 422 [M+H]^{+ 1}H NMR (400 MHz, DMSO-d₆) δ 14.03 (s, 1H), 8.23 (d, J=9.8 Hz, 1H), 8.11 (s, 1H), 7.92 (d, J=8.8 Hz, 1H), 7.21-7.12 (m, 3H), 3.68-3.49 (m, 3H), 3.37 (s, 1H), 2.69 (s, 3H), 2.45 (s, 2H), 2.04 (d, J=7.3 Hz, 1H), 1.85 (dd, J=12.9, 6.6 Hz, 1H), 1.24 (s, 3H), 0.85 (s, 1H), 0.43-0.35 (m, 2H), 0.15-0.09 (m, 2H).

### Example 127: Synthesis of Compounds 440 and 441

### Synthesis of Intermediate B324

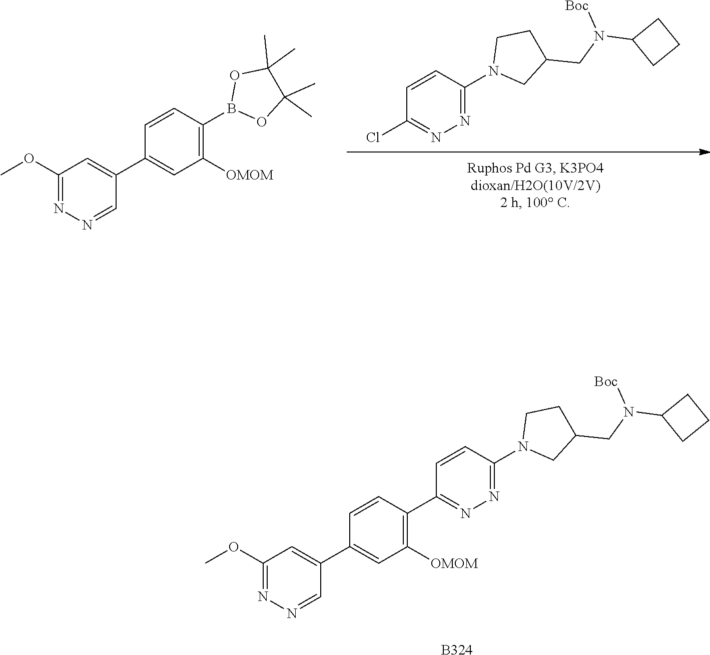

To a solution of 3-methoxy-5-[3-(methoxymethoxy)-4-(4,4,5,5-tetramethyl-1,3,2-dioxaborolan-2-yl)phenyl]pyridazine (0.20 g, 0.545 mmol, 1 equiv) and tert-butyl N-{[1-(6-chloropyridazin-3-yl)pyrrolidin-3-yl]methyl}-N-cyclobutylcarbamate (0.2 g, 0.545 mmol, 1.00 equiv) in 1,4-dioxane (2 mL) and H₂O (0.4 mL) were added K₃PO₄ (0.23 g, 1.635 mmol, 3 equiv) and RuPhos Palladacycle Gen.3 (0.05 g, 0.055 mmol, 0.1 equiv). After stirring for 2 h at 100° C. under a nitrogen atmosphere. The mixture was allowed to cool down to room temperature. The resulting mixture was filtered and the filter cake was washed with DCM (5×3 mL). the filtrate was concentrated under reduced pressure. The residue was purified by silica gel column chromatography, eluted with PE/EA (1:1) to afford tert-butyl N-cyclobutyl-N-[(1-{6-[2-(methoxymethoxy)-4-(6-methoxypyridazin-4-yl)phenyl]pyridazin-3-yl}pyrrolidin-3-yl)methyl]carbamate (200 mg, 64%) as a solid. LCMS:(ES, m/z):577[M+H]

### Synthesis of Intermediate B325

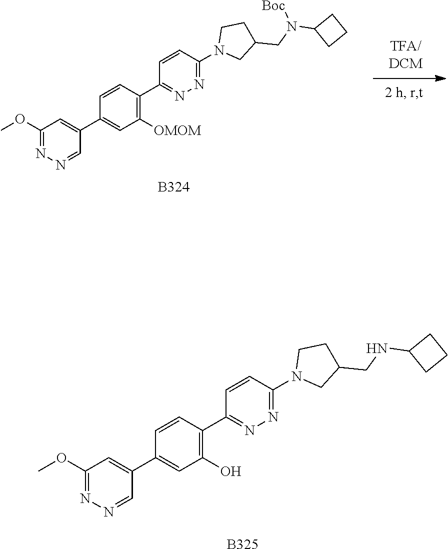

Into an 8 mL vial were added tert-butyl N-cyclobutyl-N-[(1-{6-[2-(methoxymethoxy)-4-(6-methoxypyridazin-4-yl)phenyl]pyridazin-3-yl}pyrrolidin-3-yl)methyl]carbamate (100 mg, 0.173 mmol, 1 equiv) and TFA (0.2 mL, 2.693 mmol, 15.53 equiv) DCM (1 mL, 15.731 mmol, 90.72 equiv) for 2 h at room temperature. The mixture was allowed to cool down to room temperature. The resulting mixture was concentrated under reduced pressure. The crude product (80 mg) was purified by Prep-HPLC with the following conditions (Column: Kinetex EVO C18 Column, 30*150, 5 um; Mobile Phase A: Water (10 mmol/L NH₄HCO₃), Mobile Phase B: ACN; Flow rate: 60 mL/min; Gradient: 15% B to 50% B in 10 min, 50% B; Wave Length: UV 220 nm; RT1(min): 7.35; Number Of Runs: 0) to afford 2-(6-{3-[(cyclobutylamino)methyl]pyrrolidin-1-yl}pyridazin-3-yl)-5-(6-methoxypyridazin-4-yl)phenol (25 mg, 33%) as a liquid. LCMS:(ES, m/z):433[M+H]

### Synthesis of Compound 440

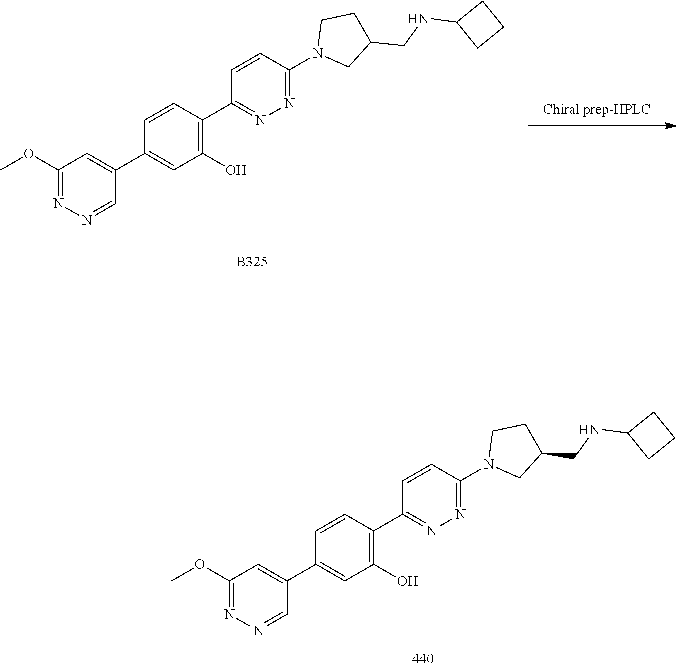 [SMILES: COc1cc(-c2ccc(-c3ccc(N4CCC(CNC5CCC5)C4)nn3)c(O)c2)cnn1 ; "B325" ; COc1cc(-c2ccc(-c3ccc(N4CC[C@@H](CNC5CCC5)C4)nn3)c(O)c2)cnn1]

2-(6-{3-[(cyclobutylamino)methyl]pyrrolidin-1-yl}pyridazin-3-yl)-5-(6-methoxypyridazin-4-yl)phenol (6.2 mg, 0.014 mmol, 1 equiv)was purified by Chiral-Prep-HPLC with the following conditions (2 #SHIMADZU (HPLC-01)): Column, CHIRAL ART Cellulose-SB, 2×25 cm, 5 um; mobile phase, MtBE(0.10% DEA and MeOH- (hold 40% MeOH- in 10.5 min); Detector, UV254 nm. This resulted in 2-{6-[(3S)-3-[(cyclobutylamino)methyl]pyrrolidin-1-yl]pyridazin-3-yl}-5-(6-methoxypyridazin-4-yl)phenol (6.2 mg, 99%) as a powder. LCMS:(ES, m/z):433[M+H] 1HNMR: (400 MHz, DMSO-d₆) δ 14.13 (s, 1H), 9.36 (d, J=1.9 Hz, 1H), 8.32 (d, J=9.9 Hz, 1H), 8.06 (d, J=8.3 Hz, 1H), 7.56 (d, J=1.9 Hz, 1H), 7.53-7.45 (m, 2H), 7.20 (d, J=9.8 Hz, 1H), 4.09 (s, 3H), 3.73-3.62 (m, 2H), 3.5-3.42 (m, 1H), 3.33 (s, 2H), 2.68(S, 1H), 2.51 (s, 2H), 2.12 (q, J=8.4, 7.9 Hz, 3H), 1.83-1.72 (m, 3H), 1.65-1.54 (m, 2H), 1.24 (s, 1H).

### Synthesis of Compound 441

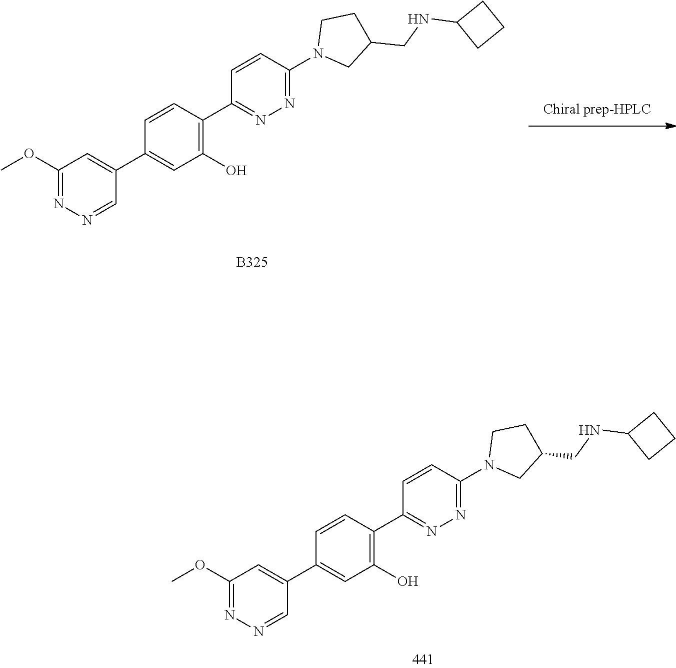 [SMILES: COc1cc(-c2ccc(-c3ccc(N4CCC(CNC5CCC5)C4)nn3)c(O)c2)cnn1 ; "B325" ; COc1cc(-c2ccc(-c3ccc(N4CC[C@H](CNC5CCC5)C4)nn3)c(O)c2)cnn1]

2-(6-{3-[(cyclobutylamino)methyl]pyrrolidin-1-yl}pyridazin-3-yl)-5-(6-methoxypyridazin-4-yl)phenol (1 equiv)was purified by Chiral-Prep-HPLC with the following conditions (2 #SHIMADZU (HPLC-01)): Column, CHIRAL ART Cellulose-SB, 2×25 cm, 5 um; mobile phase, MtBE(0.1% DEA and MeOH- (hold 40% MeOH- in 10.5 min); Detector, UV254 nm. This resulted in 2-{6-[(3S)-3-[(cyclobutylamino)methyl]pyrrolidin-1-yl]pyridazin-3-yl}-5-(6-methoxypyridazin-4-yl)phenol as a powder. LCMS:(ES, m/z):433[M+H]: ¹H NMR: (400 MHz, DMSO-d₆) δ 14.13 (s, 1H), 9.36 (d, J=1.9 Hz, 1H), 8.32 (d, J=9.9 Hz, 1H), 8.06 (d, J=8.3 Hz, 1H), 7.56 (d, J=1.9 Hz, 1H), 7.53-7.45 (m, 2H), 7.20 (d, J=9.8 Hz, 1H), 4.09 (s, 3H), 3.73-3.62 (m, 2H), 3.5(s, 1H), 3.33 (s, 2H), 2.68 (S, 1H), 2.51 (s, 2H), 2.12 (q, J=8.4, 7.9 Hz, 3H), 1.83-1.72 (m, 3H), 1.60-1.43 (m, 2H), 1.24 (s, 1H) Example 128: Synthesis of Compounds 449 and 450

### Synthesis of Intermediates B326 and B327

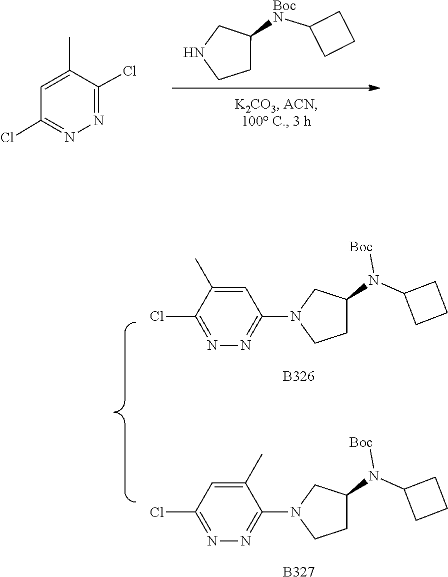

A mixture of 3,6-dichloro-4-methylpyridazine (2.2 g, 13.497 mmol, 1 equiv) and tert-butyl N-(3S)-cyclobutyl-N-(pyrrolidin-3-yl)carbamate (3.24 g, 13.497 mmol, 1 equiv), K₂CO₃ (5.60 g, 40.491 mmol, 3 equiv) in CH₃CN (20 mL) was stirred for 3 h at 100° C. under nitrogen atmosphere. The reaction was quenched by the addition of Water (10 mL) at room temperature. The resulting mixture was extracted with EtOAc (3×20 mL). The combined organic layers were washed with salt water (2×10 mL), dried over anhydrous Na₂SO₄. After filtration, the filtrate was concentrated under reduced pressure. The residue was purified by silica gel column chromatography, eluted with PE/EA (5:1) to afford the mixture product of tert-butyl N-(3S)-[1-(6-chloro-4-methylpyridazin-3-yl)pyrrolidin-3-yl]-N-cyclobutylcarbamate and tert-butyl N-(3S)-(1-(6-chloro-5-methylpyridazin-3-yl)pyrrolidin-3-yl)(cyclobutyl)carbamate (0.9 g, mixture, 18%) as an oil. LCMS: (ES, m/z): 367 [M+H]⁺

### Synthesis of Intermediates B328 and B329

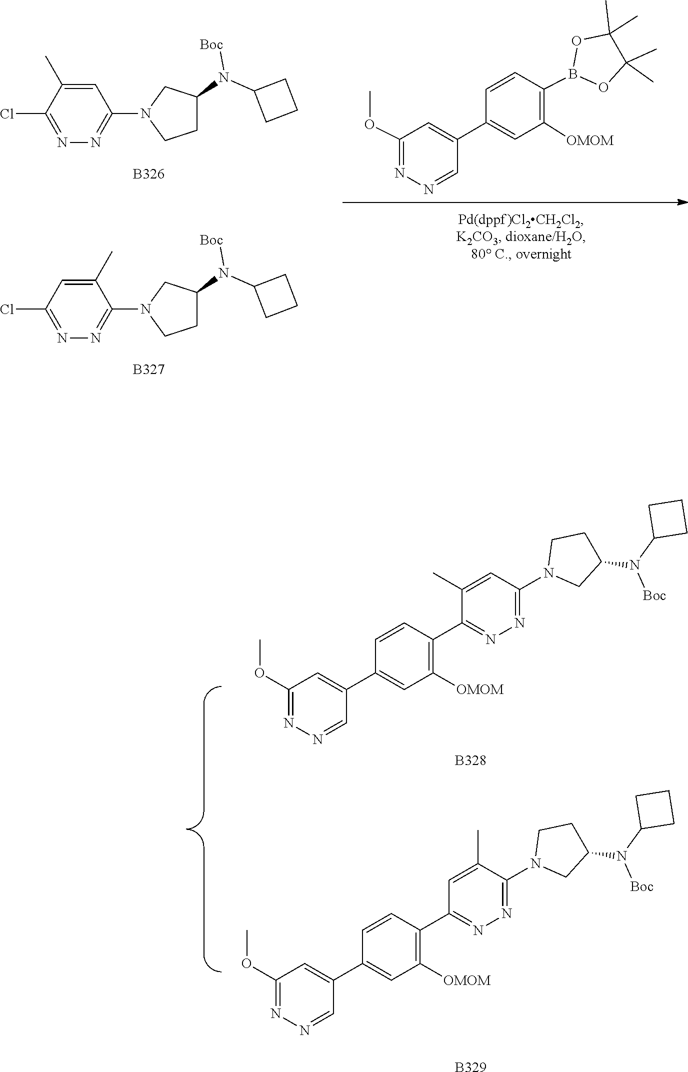

To a solution of the mixture of tert-butyl N-[(3S)-1-(6-chloro-4-methylpyridazin-3-yl)pyrrolidin-3-yl]-N-cyclobutylcarbamate and tert-butyl N-(3S)-(1-(6-chloro-5-methylpyridazin-3-yl)pyrrolidin-3-yl)(cyclobutyl)carbamate (100 mg, mixture, 0.273 mmol, 1 equiv) and 3-methoxy-5-[3-(methoxymethoxy)-4-(4,4,5,5-tetramethyl-1,3,2-dioxaborolan-2-yl)phenyl]pyridazine (101.46 mg, 0.273 mmol, 1 equiv) in dioxane (1.5 mL) and H₂O (0.3 mL) were added K₂CO₃ (113.01 mg, 0.819 mmol, 3 equiv) and Pd(dppf)Cl₂·CH₂Cl₂ (22.2 mg, 0.027 mmol, 0.1 equiv). After stirring for overnight at 80° C. under a nitrogen atmosphere, the resulting mixture was concentrated under reduced pressure. The residue was purified by silica gel column chromatography, eluted with PE/EA (5:1) to afford the mixture product of tert-butyl N-cyclobutyl-N-[(3S)-1-{6-[2-(methoxymethoxy)-4-(6-methoxypyridazin-4-yl)phenyl]-4-methylpyridazin-3-yl}pyrrolidin-3-yl]carbamate and tert-butyl (S)-cyclobutyl(1-(6-(2-(methoxymethoxy)-4-(6-methoxypyridazin-4-yl)phenyl)-5-methylpyridazin-3-yl)pyrrolidin-3-yl)carbamate (100 mg, mixture, 64%) as a solid. LCMS: (ES, m/z): 576 [M+H]⁺

### Synthesis of Compounds 449 and 450

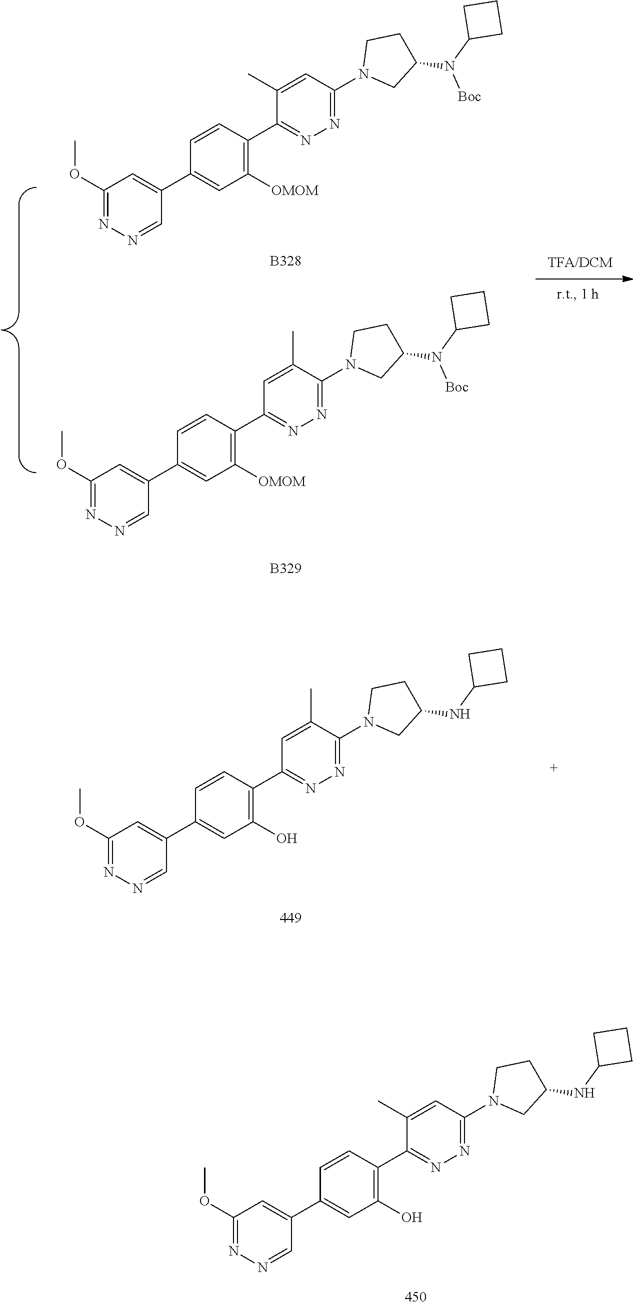

A solution of the mixture of tert-butyl N-cyclobutyl-N-[(3S)-1-{6-[2-(methoxymethoxy)-4-(6-methoxypyridazin-4-yl)phenyl]-4-methylpyridazin-3-yl}pyrrolidin-3-yl]carbamate and tert-butyl (S)-cyclobutyl(1-(6-(2-(methoxymethoxy)-4-(6-methoxypyridazin-4-yl)phenyl)-5-methylpyridazin-3-yl)pyrrolidin-3-yl)carbamate (100 mg, mixture, 0.173 mmol, 1 equiv) and TFA (0.5 mL) in DCM (2 mL) was stirred for 1 h at room temperature. The resulting mixture was concentrated under reduced pressure. The crude product (60 mg) was purified by Prep-HPLC with the following conditions (Column: Kinetex EVO C18 Column, 30*150, 5 um; Mobile Phase A: Water (10 mmol/L NH₄HCO₃), Mobile Phase B: ACN; Flow rate: 60 mL/min; Gradient: 10% B to 55% B in 10 min, 55% B; Wave Length: UV 220 nm; RT1(min): 9.13; Number Of Runs: 0) to afford 2-{6-[(3S)-3-(cyclobutylamino)pyrrolidin-1-yl]-5-methylpyridazin-3-yl}-5-(6-methoxypyridazin-4-yl)phenol (6.6 mg, 8.65%) as a yellow solid and (S)-2-(6-(3-(cyclobutylamino)pyrrolidin-1-yl)-4-methylpyridazin-3-yl)-5-(6-methoxypyridazin-4-yl)phenol (25.0 mg) as a solid. Compound 449: LCMS: (ES, m/z): 433 [M+H]^{+ 1}H NMR: (400 MHz, DMSO-d₆): δ 14.21 (s, 1H), 9.35 (d, J=1.9 Hz, 1H), 8.15 (s, 1H), 8.04 (d, J=8.3 Hz, 1H), 7.56 (d, J=1.9 Hz, 1H), 7.49 (d, J=7.0 Hz, 2H), 4.08 (s, 3H), 3.83-3.74 (m, 2H), 3.68 (d, J=8.7 Hz, 1H), 3.30 (s, 3H), 2.51 (d, J=1.8 Hz, 3H), 2.13 (s, 2H), 2.01 (dd, J=12.0, 6.2 Hz, 1H), 1.74 (dd, J=12.0, 6.5 Hz, 1H), 1.69 (s, 3H), 1.59 (s, 2H).Compound 450: LCMS: (ES, m/z): 433 [M+H]^{+ 1}H NMR: (400 MHz, DMSO-d₆) δ 10.31 (s, 1H), 9.25 (d, J=1.9 Hz, 1H), 7.47-7.31 (m, 4H), 6.75 (s, 1H), 4.09 (s, 3H), 3.59 (tt, J=14.8, 6.5 Hz, 2H), 3.49-3.31 (m, 2H), 3.29-3.18 (m, 2H), 2.15 (s, 4H), 2.20-2.03 (m, 2H), 1.80 (dq, J=13.4, 7.0 Hz, 1H), 1.71 (s, 2H), 1.75-1.49 (m, 2H).

### Example 129: Synthesis of Compound 451 and 452

### Synthesis of Intermediate 330

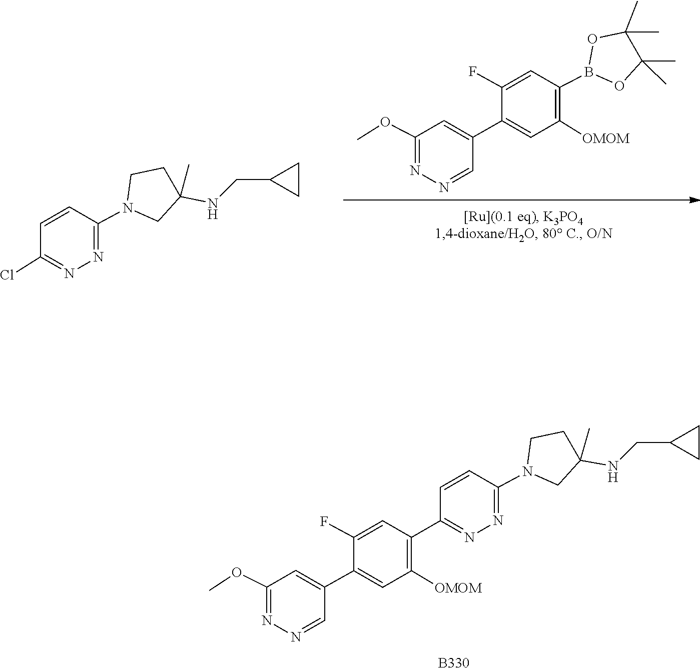

A solution of 1-(6-chloropyridazin-3-yl)-N-(cyclopropylmethyl)-3-methylpyrrolidin-3-amine (500 mg, 1.874 mmol, 1 equiv) and 5-[2-fluoro-5-(methoxymethoxy)-4-(4,4,5,5-tetramethyl-1,3,2-dioxaborolan-2-yl)phenyl]-3-methoxypyridazine (1828.45 mg, 4.685 mmol, 2.5 equiv), K₃PO₄ (1193.53 mg, 5.622 mmol, 3 equiv) and RuCl₂(p-cymene)(R-BINAP) (148.94 mg, 0.187 mmol, 0.1 equiv) in 1,4-dioxane/H₂O=5:1 was stirred for overnight at 80° C. under nitrogen atmosphere. The resulting mixture was concentrated under reduced pressure. The residue was purified by silica gel column chromatography, eluted with CH₂Cl₂/MeOH (10:1) to afford N-(cyclopropylmethyl)-1-{6-[5-fluoro-2-(methoxymethoxy)-4-(6-methoxypyridazin-4-yl)phenyl]pyridazin-3-yl}-3-methylpyrrolidin-3-amine (379 mg, 41%) as an oil.

### Synthesis of Intermediate B331

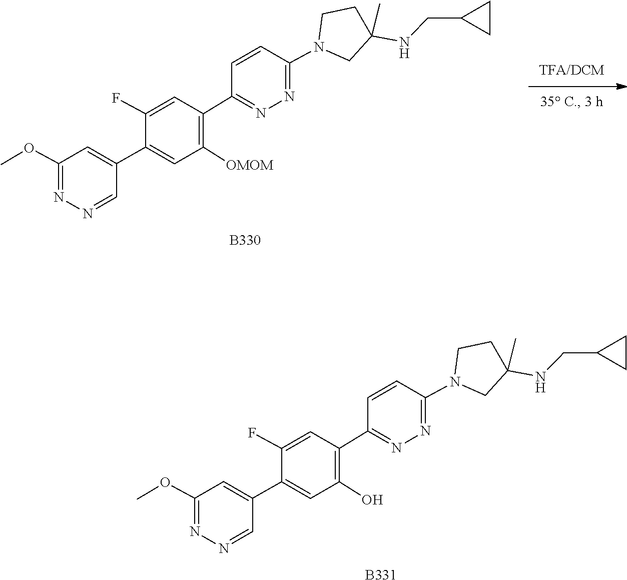

A solution of N-(cyclopropylmethyl)-1-{6-[5-fluoro-2-(methoxymethoxy)-4-(6-methoxypyridazin-4-yl) phenyl]pyridazin-3-yl}-3-methylpyrrolidin-3-amine (390 mg, 0.789 mmol, 1 equiv) and TFA (899.14 mg, 7.890 mmol, 10 equiv) in CH₂Cl₂(10 mL) was stirred for 3 h at 35° C. under air atmosphere. This resulted in 2-(6-{3-[(cyclopropylmethyl)amino]-3-methylpyrrolidin-1-yl}pyridazin-3-yl)-4-fluoro-5-(6-methoxypyridazin-4-yl)phenol (240 mg, 68%) as a solid.

### Synthesis of Compounds 451 and 452

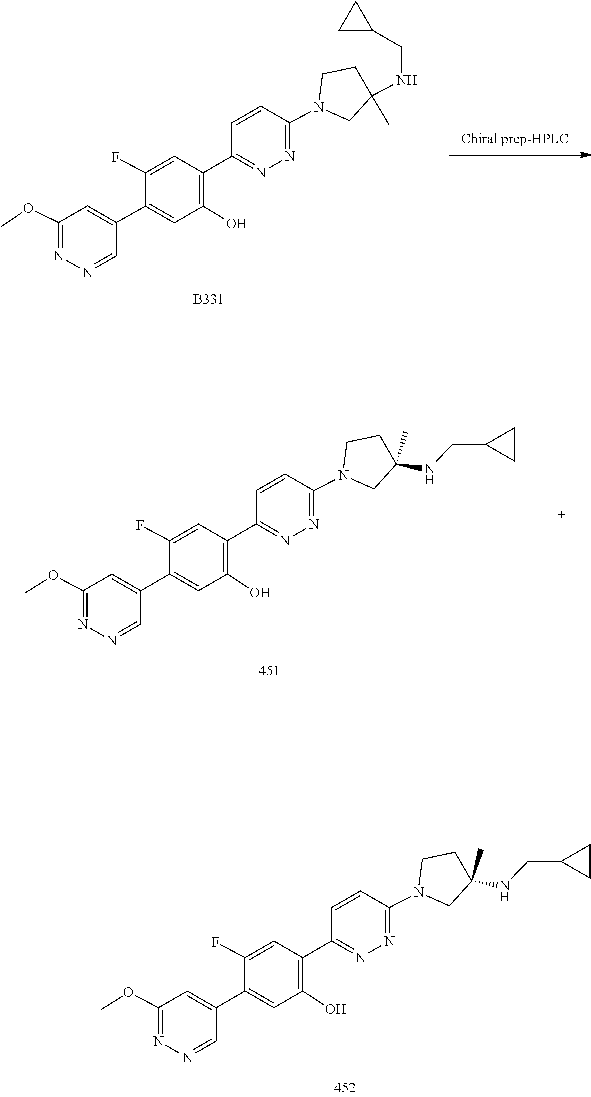 [SMILES: COc1cc(-c2cc(O)c(-c3ccc(N4CCC(C)(NCC5CC5)C4)nn3)cc2F)cnn1 ; "B331" ; COc1cc(-c2cc(O)c(-c3ccc(N4CC[C@@](C)(NCC5CC5)C4)nn3)cc2F)cnn1 ; COc1cc(-c2cc(O)c(-c3ccc(N4CC[C@](C)(NCC5CC5)C4)nn3)cc2F)cnn1]

The residue was purified by reversed-phase flash chromatography with the following conditions: (Column: CHIRALPAK IF-3, 4.6*50 mm, 3 m; Mobile Phase A: MtBE(0.1% DEA):MeOH=70:30; Flow rate: 1 mL/min; Gradient: 0% B to 0% B; Injection Volume: Sul mL) to afford 2-{6-[(3R)-3-[(cyclopropylmethyl)amino]-3-methylpyrrolidin-1-yl]pyridazin-3-yl}-4-fluoro-5-(6-methoxypyridazin-4-yl)phenol (26.9 mg, 11.04%) and 2-{6-[(3S)-3-[(cyclopropylmethyl)amino]-3-methylpyrrolidin-1-yl]pyridazin-3-yl}-4-fluoro-5-(6-methoxypyridazin-4-yl)phenol (50 mg, 20%) as a solid. Compound 451: LCMS (ES, m/z): 451 [M+H]^{+ 1}H NMR: (400 MHz, DMSO-d₆) δ 13.79 (s, 1H), 9.16 (t, J=1.9 Hz, 1H), 8.30 (d, J=9.8 Hz, 1H), 7.99 (d, J=12.5 Hz, 1H), 7.46-7.41 (m, 1H), 7.29 (d, J=6.9 Hz, 1H), 7.17 (d, J=9.7 Hz, 1H), 4.09 (s, 3H), 3.35 (m, 4H), 2.43 (d, J=6.6 Hz, 2H), 2.03 (m, 1H), 1.83 (dt, J 12.3, 7.3 Hz, 1H), 1.72 (s, 1H), 1.23 (s, 3H), 0.88-0.78 (m, 1H), 0.43-0.34 (m, 2H), 0.14-0.06 (m, 2H). Compound 452: LCMS (ES, m/z): 451 [M+H]⁺¹H NMR (400 MHz, DMSO-d₆) δ 13.80 (s, 1H), 9.16 (t, J=1.9 Hz, 1H), 8.31 (d, J=9.9 Hz, 1H), 7.99 (d, J=12.5 Hz, 1H), 7.44 (dd, J=1.8, 0.9 Hz, 1H), 7.29 (d, J=6.9 Hz, 1H), 7.17 (d, J=9.7 Hz, 1H), 4.09 (s, 3H), 3.58 (s, 3H), 3.48 (s, 1H), 2.44 (d, J=6.7 Hz, 3H), 2.04 (dt, J=12.9, 6.8 Hz, 1H), 1.84 (dt, J=12.9, 7.2 Hz, 1H), 1.23 (s, 3H), 0.88-0.78 (m, 1H), 0.38 (dt, J=8.7, 2.8 Hz, 2H), 0.15-0.07 (m, 2H).

### Example 130: Exemplary Splicing Assay for Monitoring Expression Levels of Splice Variants

Compounds described herein were used to modulate RNA transcript abundance in cells. The expression of a target mRNA was measured by detecting the formation of an exon-exon junction in the canonical transcript (CJ). A compound mediated exon-inclusion event was detected by observing an increase in formation of a new junction with an alternative exon (AJ). Real-time qPCR assays were used to detect these splicing switches and interrogate the potency of various compounds towards different target genes. A high-throughput real time quantitative PCR (RT-qPCR) assay was developed to measure these two isoforms of the mRNA (CJ and AJ) for exemplary genes, such as HTT, SMN2, and MYB, together with a control housekeeping gene, GAPDH or GUSB or PPIA, used for normalization. Briefly, the A673 or K562 cell line was treated with various compounds described herein (e.g., compounds of Formula (I), (II), (III), (IV)). After treatment, the levels of the HTT, MYB, or SMN2 mRNA targets were determined from each sample of cell lysate by cDNA synthesis followed by qPCR.

### Materials:

Cells-to-C_{T} 1-step kit: ThermoFisher A25602, Cells-to-C_{T} lysis reagent: ThermoFisher 4391851C, TaqMan™ Fast Virus 1-Step Master Mix: ThermoFisher 4444436
GAPDH: VIC-PL, ThermoFisher 4326317E (Assay: Hs99999905_ml)—used for K562/suspension cell lines
GUSB: VIC-PL, ThermoFisher 4326320E (Assay: Hs99999908_ml)—used for K562/suspension cell lines
PPIA: VIC-PL, ThermoFisher 4326316E (Assay: Hs99999904_ml)—used for A673/adherent cell lines

### Probe/Primer Sequences

Canonical junction (CJ)
HTT Primer 1:
TCCTCCTGAGAAAGAGAAGGAC
HTT Primer 2:
GCCTGGAGATCCAGACTCA
HTT CY5-Probe:
/5Cy5/TGGCAACCCTTGAGGCCCTGTCCT/3IAbRQSp/
MYB Primer 1:
CCTCATTGGTCACAAATTGACTG
MYB Primer 2:
TGGAGAGCTTTCTAAGATTGACC
MYB CY5-Probe:
/5Cy5/AGGAAAATACTGTTTTTAGAACCCCAG/3IAbRQSp/
Alternative junction (AJ)
HTT Primer 1:
TCCTGAGAAAGAGAAGGACATTG
HTT Primer 2:
CTGTGGGCTCCTGTAGAAATC
HTT FAM-Probe:
/56-FAM/TGGCAACCC/ZEN/TTGAGAGGCAAGCCCT/3IABkFQ/
MYB Primer 1:
CAACACCATTTCATAGAGACCAGAC
MYB Primer 2:
GTTCTAAAATCATCCCTTGGCTTCTAAT
MYB FAM-Probe:
/56-FAM/AAATACTGT/ZEN/ATAGGACCTCTTCTGACATCC/
3IABkFQ/

### Description

The A673 cell line was cultured in DMEM with 10% FBS. Cells were diluted with full growth media and plated in a 96-well plate (15,000 cells in 100ul media per well). The plate was incubated at 37° C. with 5% CO₂ for 24 hours to allow cells to adhere. An 11-point 3-fold serial dilution of the compounds was made in DMSO then diluted in media in an intermediate plate. Compounds were transferred from the intermediate plate to the cell plate with the top dose at a final concentration of 10 uM in the well. Final DMSO concentration was kept at or below 0.25%. The cell plate was returned to the incubator at 37° C. with 5% CO₂ for an additional 24 hours.

The K562 cell line was cultured in IMDM with 10% FBS. For K562, cells were diluted with full growth media and plated in either a 96-well plate (50,000 cells in 50 uL media per well) or a 384-well plate (8,000-40,000 cells in 45 uL media per well). An 11-point 3-fold serial dilution of the compounds were made in DMSO then diluted in media in an intermediate plate.

Compound was transferred from the intermediate plate to the cell plate with the top dose at a final concentration of 10 uM in the well. Final DMSO concentration was kept at or below 0.25%.

Final volume was 100 uL for 96-well plate and 50 uL for 384-well plate. The cell plate was then placed in an incubator at 37° C. with 5% CO₂ for 24 hours.

The cells were then gently washed with 50 uL-100 uL cold PBS before proceeding to addition of lysis buffer. 30 uL-50 μL of room temperature lysis buffer with DNAse I (and optionally RNAsin) was added to each well. Cells were shaken/mixed thoroughly at room temperature for 5-10 minutes for lysis to take place and then 3 uL-5 μL of room temperature stop solution was added and wells were shaken/mixed again. After 2-5 minutes, the cell lysate plate was transferred to ice for RT-qPCR reaction setup. The lysates could also be frozen at −80° C. for later use.

In some cases, a direct lysis buffer was used. An appropriate volume of 3× lysis buffer (10 mM Tris, 150 mM NaCl, 1.5%-2.5% Igepal and 0.1-1 U/uL RNAsin, pH 7.4) was directly added to either K562 or A673 cells in media and mixed by pipetting 3 times. The plates were then incubated at room temperature with shaking/rocking for 20-50 minutes to allow for lysis to take place. After this time, the cell lysate plate was transferred to ice to set up for the RT-qPCR reactions. The lysates could also be frozen at −80° C. for later use.

To set up 10 uL RT-qPCR reactions, cell lysates were transferred to 384-well qPCR plates containing the master mix according to the table below. The plates were sealed, gently vortexed, and spun down before the run. The volumes were adjusted accordingly in some instances where the reaction was carried in 20 μL. The table below summarizes the components of the RT-qPCR reactions:

| Component | 1X |
|---|---|
| Taqman 1-step RT-qPCR mix (4X) | 2.5 |
| 20X AJ Primers + Probe (FAM) | 0.5 |
| 20X CJ Primers + Probe (CY5) | 0.5 |
| 20X PPIA Control (VIC) | 0.5 |
| Cell lysate (1X) | 1-2 |
| H₂O | 4-5 |
| Total volume | 10 |

The RT-qPCR reaction was performed using a QuantStudio (ThermoFisher) under the following fast cycling conditions. All samples and standards were analyzed at least in duplicate. In some instances, bulk room temperature (RT) step of 5-10 minutes was completed for all plates before proceeding with qPCR. The table below summarizes the PCR cycle:

| Step | # cycles | Temp. | Time | |
|---|---|---|---|---|
| RT step | 1 | 50° C. | 5 | min |
| RT inactivation/initial | 1 | 95° C. | 20 | sec |
| denaturation | | | | |
| Amplification | 40 | 95° C. | 3 | sec |
| | | 60° C. | 30 | sec |

The data analysis was performed by first determining the ΔCt vs the housekeeper gene. This ΔCt was then normalized against the DMSO control (ΔΔCt) and converted to RQ (relative quantification) using the 2\hat{ }(-ΔΔCt) equation. The RQ were then converted to a percentage response by arbitrarily setting an assay window of 3.5 and 4.0 ΔCt for HTT-CJ and MYB-CJ respectively and an assay window of 9 and 3 ΔCt for HTT-AJ and MYB-AJ in 96 well format (50,000 K562 cells/well and 15,000 A673 cells per well) and an assay window of 3 and 4 ΔCt for HTT-CJ and MYB-CJ respectively and an assay window of 5 and 3 ΔCt for HTT-AJ and MYB-AJ respectively in 384 well format (8,000 K562 cells/well example). These assay windows correspond to the maximal modulation observed at high concentration of the most active compounds. The percentage response was then fitted to the 4 parametric logistic equation to evaluate the concentration dependence of compound treatment. The increase in AJ mRNA is reported as AC₅₀ (compound concentration having 500 response in AJ increase) while the decrease in CJ mRNA levels is reported as IC₅₀ (compound concentration having 50% o response in CJ decrease).

A summary of these results is illustrated in Tables 3A and 3B, wherein “A” represents an AC₅₀/IC₅₀ of less than 100 nM; “B” represents an AC₅₀/IC₅₀ of between 100 nM and 1 μM; and “C” represents an AC₅₀/IC₅₀ of between 1 μM and 10 μM; and “D” represents an AC₅₀/IC₅₀ of greater than 10 μM.

TABLE 3A
Modulation of RNA Splicing by Exemplary Compounds.

| Compound | HTT | HTT | MYB | MYB |
|---|---|---|---|---|
| No. | CJ | AJ | CJ | AJ |
| 100 | A | A | B | B |
| 101 | A | A | A | A |
| 102 | D | D | D | D |
| 103 | A | A | B | B |
| 104 | A | A | B | B |
| 105 | A | A | B | B |
| 106 | A | A | B | B |
| 107 | A | A | A | A |
| 108 | A | A | B | B |
| 109 | C | D | D | D |
| 110 | D | D | D | D |
| 111 | A | A | A | A |
| 112 | A | A | B | A |
| 113 | A | A | A | A |
| 114 | A | A | B | B |
| 115 | A | A | A | A |
| 116 | A | A | B | B |
| 117 | C | C | D | D |
| 119 | A | A | A | A |
| 120 | A | A | A | A |
| 146 | A | A | A | A |
| 161 | B | B | B | B |
| 208 | B | B | C | B |
| 209 | A | A | A | A |
| 210 | A | A | A | A |
| 211 | B | B | C | C |
| 212 | A | A | A | A |
| 213 | B | B | B | C |
| 214 | A | A | B | B |
| 215 | A | A | B | B |
| 216 | A | B | B | B |
| 217 | B | B | B | C |
| 218 | A | A | A | A |
| 219 | C | C | C | C |
| 220 | B | B | B | B |
| 221 | B | B | B | C |
| 222 | B | B | B | C |
| 223 | B | B | B | B |
| 224 | C | D | D | D |
| 225 | D | D | D | D |
| 226 | A | B | B | B |
| 227 | A | A | B | B |
| 228 | B | B | B | B |
| 229 | A | A | B | B |
| 230 | B | B | C | C |
| 231 | A | A | B | B |
| 232 | A | B | B | B |
| 234 | A | A | A | A |
| 235 | A | A | B | B |
| 236 | B | B | B | B |
| 237 | A | A | B | B |
| 238 | A | B | B | B |
| 239 | A | A | B | B |
| 241 | A | B | B | B |
| 242 | A | B | B | B |
| 261 | A | A | B | B |
| 262 | A | A | A | A |
| 263 | A | A | B | B |
| 264 | A | B | B | B |
| 265 | A | A | A | A |
| 266 | A | A | B | B |
| 267 | A | A | A | A |
| 268 | B | C | D | D |
| 269 | A | A | A | B |
| 270 | D | D | D | D |
| 271 | A | A | A | B |
| 272 | A | A | B | B |
| 273 | B | B | B | B |
| 274 | A | B | B | B |
| 275 | A | A | B | B |
| 276 | A | B | B | B |
| 277 | C | D | C | D |
| 278 | C | D | C | D |
| 279 | C | D | D | D |
| 280 | C | C | C | D |
| 281 | B | B | C | C |
| 282 | | | | |
| 283 | A | A | B | B |
| 284 | A | A | B | B |
| 285 | A | A | B | B |
| 286 | A | B | C | C |
| 287 | A | A | A | A |
| 288 | A | B | B | B |
| 289 | A | B | B | B |
| 290 | B | B | B | B |
| 291 | C | D | D | D |
| 292 | D | D | D | D |
| 293 | B | B | C | B |
| 294 | A | B | B | B |
| 295 | A | A | B | B |
| 296 | B | B | B | B |
| 297 | A | A | A | A |
| 298 | A | B | B | B |
| 299 | A | A | B | B |
| 300 | A | A | B | B |
| 301 | D | D | D | D |
| 302 | | | | |
| 303 | B | B | D | D |
| 304 | C | C | C | C |
| 305 | B | B | C | C |
| 306 | C | C | C | C |
| 307 | C | C | D | D |
| 308 | A | B | C | B |
| 309 | A | B | C | C |
| 310 | A | B | B | B |
| 311 | | | | |
| 312 | | | | |
| 313 | B | C | D | C |
| 314 | A | B | C | B |
| 315 | C | C | D | D |
| 316 | B | B | C | B |
| 317 | A | B | B | B |
| 318 | B | B | C | D |
| 319 | B | B | C | B |
| 320 | B | B | B | B |
| 321 | B | B | C | C |
| 322 | A | A | A | A |
| 323 | A | A | A | A |
| 324 | A | A | C | B |
| 325 | C | C | C | C |
| 326 | A | B | B | B |
| 327 | B | B | C | B |
| 328 | A | A | B | B |
| 329 | A | A | B | B |
| 333 | C | D | D | D |
| 334 | A | B | B | C |
| 335 | A | B | B | C |
| 336 | A | A | A | A |
| 337 | A | A | A | A |
| 338 | A | A | A | A |
| 339 | A | A | A | A |
| 342 | A | A | D | C |
| 343 | B | C | D | D |
| 344 | C | C | C | C |
| 345 | C | C | C | C |
| 347 | B | B | B | B |
| 348 | A | B | B | C |
| 349 | B | B | B | B |
| 350 | A | A | A | B |
| 351 | A | B | B | C |
| 352 | B | B | B | C |
| 353 | B | B | C | C |
| 354 | A | A | B | A |
| 355 | A | A | B | B |
| 356 | A | A | B | B |
| 357 | A | A | B | B |
| 358 | B | C | C | C |
| 359 | B | C | C | C |
| 360 | A | A | A | A |
| 361 | A | A | B | A |
| 362 | B | C | C | B |
| 364 | B | B | B | C |
| 365 | A | A | B | B |
| 366 | A | A | B | B |
| 367 | D | D | D | D |
| 368 | B | B | C | C |
| 369 | C | C | C | C |
| 370 | D | D | D | D |
| 371 | C | C | D | D |
| 372 | A | A | A | A |
| 373 | A | A | A | A |
| 374 | B | B | B | C |
| 375 | A | B | B | C |
| 376 | A | A | B | B |
| 377 | B | B | C | C |
| 378 | B | B | C | C |
| 379 | A | A | A | A |
| 380 | A | A | A | A |
| 381 | A | B | B | B |
| 382 | A | B | B | B |
| 383 | A | A | B | B |
| 384 | A | A | B | B |
| 385 | A | A | B | B |
| 386 | B | C | C | C |
| 387 | B | C | C | C |
| 388 | C | C | C | C |
| 389 | B | C | C | C |
| 390 | D | D | D | D |
| 391 | A | A | A | B |
| 392 | A | A | A | A |
| 393 | A | A | A | A |
| 394 | A | A | A | A |
| 395 | A | B | B | B |
| 396 | B | C | C | C |
| 397 | B | B | B | C |
| 398 | C | C | C | C |
| 399 | A | A | B | B |
| 400 | A | A | B | B |
| 401 | B | B | C | C |
| 402 | B | B | C | C |
| 403 | A | B | B | C |
| 404 | A | B | B | C |
| 405 | A | A | A | A |
| 406 | B | B | B | C |
| 407 | B | B | B | B |
| 408 | A | B | B | B |
| 409 | D | D | D | D |
| 410 | A | A | C | B |
| 411 | A | A | C | B |
| 412 | D | D | D | D |
| 413 | C | C | C | C |
| 414 | A | B | B | B |
| 415 | A | A | B | B |
| 416 | A | A | B | B |
| 417 | C | C | D | C |
| 418 | A | A | A | A |
| 419 | B | C | C | D |
| 420 | B | C | C | D |
| 421 | B | B | B | B |
| 422 | A | A | B | B |
| 423 | C | C | D | D |
| 424 | B | B | C | C |
| 425 | B | C | C | C |
| 426 | B | B | C | C |
| 427 | B | B | C | C |
| 428 | B | B | B | B |
| 429 | B | B | C | C |
| 430 | D | D | D | D |
| 431 | B | B | B | B |
| 432 | A | B | C | B |
| 433 | D | D | D | D |
| 434 | A | A | B | B |
| 435 | B | B | C | C |
| 436 | A | A | B | B |
| 437 | A | A | A | A |
| 438 | A | B | B | B |
| 439 | A | A | B | B |
| 440 | A | B | B | B |
| 441 | B | B | B | B |
| 442 | D | C | D | D |
| 443 | C | C | D | D |
| 444 | D | D | D | D |
| 445 | D | D | D | D |
| 446 | A | A | A | A |
| 447 | A | A | B | B |
| 448 | B | B | B | B |
| 449 | B | B | B | B |
| 450 | C | C | C | C |
| 451 | A | B | B | B |

TABLE 3B

| Cmpd No. | HTT CJ | HTT AJ | MYB CJ | MYB AJ |
|---|---|---|---|---|
| 800 | B | B | C | B |
| 801 | C | C | C | C |
| 805 | B | C | C | D |
| 808 | C | D | D | D |
| 809 | A | C | C | C |
| 812 | A | A | B | B |
| 813 | B | C | C | C |
| 817 | D | D | D | D |
| 818 | D | D | D | D |
| 819 | C | C | C | C |
| 820 | B | B | C | B |
| 830 | D | D | D | D |
| 832 | A | B | B | B |
| 835 | B | B | B | B |
| 836 | B | C | C | C |
| 837 | B | C | C | C |
| 843 | D | D | D | D |
| 845 | A | A | B | B |
| 856 | B | C | C | C |
| 857 | A | B | B | B |
| 858 | A | A | B | B |
| 859 | C | C | C | C |
| 862 | B | B | C | C |
| 869 | D | D | D | D |
| 870 | C | C | C | C |
| 873 | B | C | D | C |
| 874 | A | A | B | B |
| 875 | A | A | B | B |
| 881 | D | D | D | D |
| 882 | B | B | B | B |
| 883 | C | C | C | C |
| 884 | C | C | D | D |
| 892 | C | C | D | D |
| 893 | B | B | B | C |
| 894 | D | D | D | D |
| 895 | A | B | B | B |
| 896 | B | B | C | C |
| 897 | A | B | B | B |
| 898 | A | A | A | A |
| 899 | A | A | B | B |
| 900 | C | C | C | C |

Additional studies were carried out for a larger panel of genes using the protocol provided above. The junction between flanking upstream and downstream exons was used to design canonical junction qPCR assays. At least one of the forward primer, reverse primer or the CY5-labeled 5′ nuclease probe (with 3′ quencher such as ZEN/Iowa Black FQ) was designed to overlap with the exon junction to capture the CJ mRNA transcript. BLAST was used to confirm the specificity of the probeset and parameters such as melting temperature, GC content, amplicon size, and primer dimer formation are considered during their design. Data for the decrease in CJ mRNA levels for four exemplary genes (HTT, SMN2, MYB, and Target C) analyzed in this panel are reported as IC₅₀ (compound concentration having 50% response in CJ decrease).

A summary of the results from the panel is illustrated in Tables 4A and 4B, wherein “A” represents an IC₅₀ of less than 100 nM; “B” represents an IC₅₀ of between 100 nM and 1 μM; and “C” represents an IC₅₀ of between 1 μM and 10 μM; and “D” represents an IC₅₀ of greater than 10 μM.

TABLE 4A
Modulation of RNA Splicing by Exemplary Compounds

| Cmpd | | | | Target |
|---|---|---|---|---|
| No. | HTT | MYB | SMN2 | C |
| 100 | A | B | A | A |
| 101 | A | A | A | A |
| 102 | D | D | C | D |
| 103 | A | B | A | B |
| 104 | A | B | A | B |
| 105 | A | B | A | A |
| 106 | A | B | A | B |
| 107 | A | A | A | A |
| 108 | A | B | A | B |
| 109 | C | D | C | D |
| 110 | D | D | D | D |
| 111 | A | A | A | A |
| 112 | A | B | A | B |
| 113 | A | A | A | A |
| 114 | A | B | A | B |
| 115 | A | A | A | A |
| 116 | A | B | A | A |
| 117 | C | D | B | D |
| 119 | A | A | A | A |
| 120 | A | A | A | A |
| 146 | A | A | A | A |
| 161 | B | B | A | B |
| 208 | B | C | B | C |
| 209 | A | A | A | A |
| 210 | A | A | A | A |
| 211 | B | C | A | B |
| 212 | A | A | A | A |
| 213 | B | B | A | C |
| 214 | A | B | A | B |
| 215 | A | B | A | B |
| 216 | A | B | A | B |
| 217 | B | B | A | B |
| 218 | A | A | A | A |
| 219 | C | C | C | C |
| 220 | B | B | B | B |
| 221 | B | B | A | C |
| 222 | B | B | B | C |
| 223 | B | B | B | C |
| 224 | C | D | C | D |
| 225 | D | D | D | D |
| 226 | A | B | A | B |
| 227 | A | B | A | B |
| 228 | B | B | A | B |
| 229 | A | B | A | B |
| 230 | B | C | A | C |
| 231 | A | B | A | B |
| 232 | A | B | A | B |
| 261 | A | B | A | A |
| 262 | A | A | A | A |
| 263 | A | B | A | B |
| 264 | A | B | A | B |
| 265 | A | A | A | A |
| 266 | A | B | A | B |
| 267 | A | A | A | A |
| 268 | B | D | B | D |
| 269 | A | A | A | A |
| 270 | D | D | B | D |
| 271 | A | A | A | A |
| 272 | A | B | A | B |
| 273 | B | B | A | B |
| 274 | A | B | A | B |
| 275 | A | B | A | A |
| 276 | A | B | A | B |
| 277 | C | C | C | D |
| 278 | C | C | C | D |
| 279 | C | D | B | D |
| 280 | C | C | B | D |
| 281 | B | C | A | C |
| 283 | A | B | A | A |
| 284 | A | B | A | B |
| 285 | A | B | A | A |
| 286 | A | C | A | B |
| 287 | A | A | A | A |
| 288 | A | B | A | B |
| 289 | A | B | A | A |
| 290 | B | B | A | B |
| 291 | C | D | C | D |
| 292 | D | D | C | D |
| 293 | B | C | A | C |
| 294 | A | B | A | B |
| 295 | A | B | A | A |
| 296 | B | B | A | B |
| 297 | A | A | A | A |
| 298 | A | B | A | B |
| 299 | A | B | A | B |
| 300 | A | B | A | B |
| 301 | D | D | C | D |
| 303 | B | D | B | D |
| 304 | C | C | C | C |
| 305 | B | C | B | C |
| 306 | C | C | B | C |
| 307 | C | D | C | D |
| 308 | A | C | A | B |
| 309 | A | C | A | B |
| 310 | A | B | A | B |
| 313 | B | D | B | D |
| 314 | A | C | A | B |
| 315 | C | D | C | D |
| 316 | B | C | B | B |
| 317 | A | B | B | B |
| 318 | B | C | B | B |
| 319 | B | C | B | C |
| 320 | B | B | A | B |
| 321 | B | C | B | B |
| 322 | A | A | A | A |
| 323 | A | A | A | A |
| 324 | A | C | A | A |
| 325 | C | C | C | C |
| 326 | A | B | A | B |
| 327 | B | C | A | C |
| 328 | A | B | A | B |
| 329 | A | B | A | A |
| 333 | C | D | C | D |
| 334 | A | B | B | B |
| 335 | A | B | B | B |
| 336 | A | A | A | A |
| 337 | A | A | A | A |
| 338 | A | A | A | A |
| 339 | A | A | A | A |
| 342 | A | D | A | A |
| 343 | B | D | B | C |
| 344 | C | C | B | C |
| 345 | C | C | B | D |
| 347 | B | B | A | B |
| 348 | A | B | A | B |
| 349 | B | B | B | C |
| 350 | A | A | A | A |
| 351 | A | B | B | B |
| 352 | B | B | B | B |
| 353 | B | C | A | B |
| 354 | A | B | A | A |
| 355 | A | B | A | A |
| 356 | A | B | A | B |
| 357 | A | B | A | A |
| 358 | B | C | B | C |
| 359 | B | C | B | C |
| 360 | A | A | A | A |
| 361 | A | B | A | A |
| 362 | B | C | B | B |
| 364 | B | B | A | B |
| 365 | A | B | A | A |
| 366 | A | B | A | B |
| 367 | D | D | C | D |
| 368 | B | C | B | C |
| 369 | C | C | B | C |
| 370 | D | D | B | D |
| 371 | C | D | C | D |
| 372 | A | A | A | A |
| 373 | A | A | A | A |
| 374 | B | B | A | B |
| 375 | A | B | A | B |
| 376 | A | B | A | B |
| 377 | B | C | B | B |
| 378 | B | C | B | C |
| 379 | A | A | A | A |
| 380 | A | A | A | A |
| 381 | A | B | A | B |
| 382 | A | B | A | B |
| 383 | A | B | A | A |
| 384 | A | B | A | B |
| 385 | A | B | A | B |
| 386 | B | C | B | C |
| 387 | B | C | B | C |
| 388 | C | C | B | C |
| 389 | B | C | B | C |
| 390 | D | D | C | D |
| 391 | A | A | A | A |
| 392 | A | A | A | A |
| 393 | A | A | A | A |
| 394 | A | A | A | A |
| 395 | A | B | A | B |
| 396 | B | C | A | C |
| 397 | B | B | B | B |
| 398 | C | C | C | C |
| 399 | A | B | A | A |
| 400 | A | B | A | A |
| 401 | B | C | B | B |
| 402 | B | C | B | C |
| 403 | A | B | A | B |
| 404 | A | B | A | B |
| 405 | A | A | A | A |
| 406 | B | B | B | B |
| 407 | B | B | B | B |
| 408 | A | B | A | B |
| 409 | D | D | D | D |
| 410 | A | C | A | B |
| 411 | A | C | A | A |
| 412 | D | D | D | D |
| 413 | C | C | C | D |
| 414 | A | B | A | B |
| 415 | A | B | A | B |
| 416 | A | B | A | B |
| 417 | C | D | C | D |
| 418 | A | A | A | A |
| 419 | B | C | B | C |
| 420 | B | C | B | C |
| 421 | B | B | B | B |
| 422 | A | B | A | B |
| 423 | C | D | B | D |
| 424 | B | C | B | C |
| 425 | B | C | B | C |
| 426 | B | C | B | C |
| 427 | B | C | B | C |
| 428 | B | B | A | B |
| 429 | B | C | B | B |
| 430 | D | D | D | D |
| 431 | B | B | A | B |
| 432 | A | C | A | B |
| 433 | D | D | D | D |
| 434 | A | B | A | B |
| 435 | B | C | A | B |
| 436 | A | B | A | A |
| 437 | A | A | A | A |
| 438 | A | B | A | B |
| 439 | A | B | A | A |
| 440 | A | B | A | B |
| 441 | B | B | A | B |
| 442 | D | D | C | D |
| 443 | C | D | C | D |
| 444 | D | D | C | D |
| 445 | D | D | D | D |
| 446 | A | A | A | A |
| 447 | A | B | A | B |
| 448 | B | B | A | B |
| 449 | B | B | A | C |
| 450 | C | C | C | C |
| 451 | A | B | A | B |
| 452 | A | B | B | B |
| 453 | A | C | A | C |
| 454 | A | C | A | B |
| 455 | D | D | D | D |

TABLE 4B

| Cmpd No. | HTT | MYB | SMN2 | Target C |
|---|---|---|---|---|
| 800 | B | C | A | B |
| 801 | C | C | B | C |
| 805 | B | C | C | C |
| 808 | C | D | C | D |
| 809 | A | C | B | C |
| 812 | A | B | A | B |
| 813 | B | C | B | C |
| 817 | D | D | C | D |
| 818 | D | D | D | D |
| 819 | C | C | B | C |
| 820 | B | C | A | C |
| 830 | D | D | D | D |
| 832 | A | B | A | B |
| 835 | B | B | B | B |
| 836 | B | C | B | C |
| 837 | B | C | B | C |
| 843 | D | D | D | D |
| 845 | A | B | A | B |
| 856 | B | C | B | C |
| 857 | A | B | A | B |
| 858 | A | B | A | B |
| 859 | C | C | A | C |
| 862 | B | C | B | C |
| 869 | D | D | D | D |
| 870 | C | C | C | C |
| 873 | B | D | B | C |
| 874 | A | B | A | B |
| 875 | A | B | A | B |
| 881 | D | D | D | D |
| 882 | B | B | A | B |
| 883 | C | C | C | C |
| 884 | C | D | C | D |
| 892 | C | D | C | D |
| 893 | B | B | B | C |
| 894 | D | D | C | D |
| 895 | A | B | A | B |
| 896 | B | C | B | C |
| 897 | A | B | A | B |
| 898 | A | A | A | A |
| 899 | A | B | A | B |
| 900 | C | C | C | C |

### Example 131: Exemplary In Vitro Assays for Measuring Membrane Efflux and Permeability

Compounds described herein were screened for cell membrane permeability and efflux in a transwell assay using Madin-Darby Canine Kidney (MDCK) cells expressing Breast Cancer Resistance Protein (BCRP) or subclone MVDCKII cells expressing Multidrug Resistance Protein 1 (MDR1).

### Materials:

MDCK-BCRP or MIDCKII-MDR1 Cells: The Netherlands Cancer Institute, High Glucose Dulbecco's Modified Eagle's Medium (DMVEM): Gibco/Thermo Fisher Scientific, Fetal Bovine Serum (FBS): Corning, Hank's Balanced Salt Solution (HIBSS) and Trypsin/EDTA: Gibco/Thermo Fisher Scientific, HTS Transwell-96 Well Permeable Supports: Corning Corporation, Millicell Epithelial Volt-Ohm: Millipore, Cellometer® Vision: Nexcelom Bioscience LLC, Infinite 200 PRO microplate reader: Tecan, MTS2/4 orbital shaker: IKA Labortechnik, HEPES/Penicillin/Streptomycin: Solarbio.

### Description

MDCK-BCRP or MDCKII-MDR1 cells were seeded to each of the wells of the transwell plate at a density of 1.6×10⁶ cells/mL and the plate was incubated for 4-8 days with medium changes every two days. To perform the drug transport assay, cell monolayers were first washed three times with pre-warmed HBSS and then incubated with gentle shaking (150 rpm) for 30 min at 37° C. Stock solutions of test compounds (e.g., exemplary compounds of Formulas (I), (II), (III), (IV)) and control compounds (e.g., metoprolol, prazosin, and/or imatinib) were prepared in DMSO and adjusted to a working solution using HBSS. The final concentration of test compounds was 1 μM with DMSO; 0.5%. The plates were incubated at 37° C. for 2 hours. Permeability and efflux were measured according to published protocols (e.g., those described in Drug Metabolism and Disposition 36, 268-275 (2008) and Journal of Pharmaceutical Sciences 107 2225-2235 (2018)). Exemplary compounds were then analyzed and binned; an exemplary scheme is set out as follows. For the efflux ratio, A represents a ratio less than 1.5; B represents a ratio between 1.5 and 5; and C represents a ratio greater than 5. For permeability, A represents a Papp <2×10⁻⁶ cm s⁻¹; B represents a Papp between 2-6×10⁻⁶ cm s⁻¹; and C represents a Papp

### Example 132: Evaluating Effect of Exemplary Compounds on Protein Abundance

Compounds described herein were used to screen for effects on quantitative protein abundance using a HiBit assay system (Promega). Quantitative protein abundance was determined by measuring the protein levels of HiBit-tagged protein targets expressed in cell culture via luminescence using the Nano-Glo HiBiT Lytic Detection System, which uses a split complementation assay format to reconstitute NanoBiT enzyme to generate a luminescent signal. A protein abundance assay was developed such that endogenous protein targets could be modified with the HiBiT peptide tag and their abundance could be assessed after compound treatment. Briefly, K562 cell lines containing a HiBiT-modification were treated with various compounds described herein (e.g., compounds of Formulas (I), (II), (III), or (IV)). After treatment for 24 hours, the protein abundance of a specific target was determined by measuring luminescence.

### Materials:

Promega Nano-Glo HiBiT Lytic Detection System (cat #N3030)
Corning 384-well TC-treated microplates (cat #3570)
Synthego Engineered Cells Knock-In Clones

TABLE 5
Design of genetically modified HiBiT cell lines

| | | | Guide | Guide | |
|---|---|---|---|---|---|
| Cell | | Modi- | RNA | RNA cut | |
| Line | Gene | fication | Sequence | location | Donor Sequence |
| K562 | MYB | HiBiT | GCGCCA | chr6: | CGGTGCGGTCCCCGCGGCTC |
| | | | TGGCCC | 135,181,526 | TCGGCGGAGCCCCGCGCCCG |
| | | | GAAGAC | | CCGCGCCATGgtgagcggctggcgg |
| | | | CC | | ctgttcaagaagattagcGGCAGCTCC |
| | | | | | GGAGGATCTAGCGGCGCCCG |
| | | | | | AAGACCCCGGCACAGgtaacgg |
| | | | | | ggagccgggcgggcggccgaggg |
| K562 | HTT | HiBiT | CAGCTTT | chr4: | CGAGTCGGCCCGAGGCCTCC |
| | | | TCCAGG | 3,074,830 | GGGGACTGCCGTGCCGGGCG |
| | | | GTCGCC | | GGAGACCGCCATGgtgagcggctg |
| | | | A | | gcggctgttcaagaagattagcGGCAGC |
| | | | | | TCCGGAGGATCTAGCGGCGC |
| | | | | | GACCCTGGAAAAGCTGATGA |
| | | | | | AGGCCTTCGAGTCCCTCAAG |
| | | | | | TCCTTCCA |

### Description

Cells were maintained in IMDM with 10o FBS. Before the assay, cells were diluted with phenolphthalein-free growth media (IMDM+100 FBS media) and were seeded in a 384-well plate at a density of 10000 cells/well (for each cell line listed in Table 5). Each compound was prepared as a 10-point 3-fold serial dilution in DMSO with the top dose at a final concentration of 10 M in the well. Unmodified K562 cells were added at the previously specified density with DMSO to serve as an assay baseline and positive control (PC) and DMSO only with the respective modified cell lines was added to the negative control (NC) columns. Final DMSO concentration was kept at or below 0.25% o. Treated cell plates were placed in an incubator at 37° C. with 500 CO₂ for 24 hours. After 24 hours, 25 μL of Complete HiBit Lytic reagent was added to each well at room temperature (e.g. one plate requiring 10 mL Lytic Buffer, 100 μL LgBiT Protein, 200 μL Lytic Substrate), shaken for 5 minutes at 600 RPM, then left to sit for 10 minutes for signal to stabilize before reading on a Spark Cyto plate reader (Tecan) with a 500 ms measurement time.

To determine compound effects on protein abundance of each target in Table 5, the percent response for each respective cell line was calculated at each compound concentration as follows:

$$\% response = 100 * ( S - PC ) / ( NC - PC )$$

For the normalized response at each concentration, a four-parameter logistical regression was fit to the data and the response was interpolated at the 50% value to determine a concentration for protein abundance at 50% (IC₅₀) the untreated control. A summary of the results for protein abundance can be prepared and labeled, with compounds following in the following representative categories: A represents <100 nM; B represents 100-1000 nM; C represents 1000-9999 nM; and D represents greater than 10 μM.

### Example 133: Investigating Effect of Exemplary Compounds on Cell Viability

Compounds described herein were screened for toxicity in K562 (human chronic myelogenous leukemia) and SH-SY5Y (human neuroblastoma) cells using a Cell Titer Glo 2.0 assay.

### Materials

Promega CellTiter-Glo® 2.0 Cell Viability Assay (cat #G9241)
Corning 384-well TC-treated microplates (cat #3570)

### Description

Cells were plated at 500 cells/well (K562 cells) in 45 μL of IMDM supplemented with 10% FBS in a 384-well opaque plate. Wells containing only medium were used as a blank control. Test compounds (e.g., compounds of Formula (I), (II), (III), (IV)) were first serially diluted in DMSO then diluted 1:100 with IMIDM+10% FBS. The final concentration of DMSO was 0.1% in each well. The cells were incubated for 72 hours at 37° C. and 5% CO₂ before assaying with Cell Titer Glo 2.0 reagent.

Exemplary compounds were tested and found to fall within the following ranges: compounds labeled “A” represent <100 nM; “B” represent 100-1000 nM; “C” represent 1000-9999 nM; and “D” represent greater than 10 μM in K562 cells.

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A compound of Formula (I): 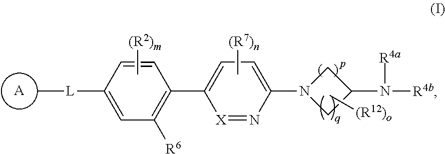
or a pharmaceutically acceptable salt,
solvate, hydrate, tautomer, or stereoisomer thereof, wherein:
A is heteroaryl optionally substituted with one or more R¹;
L is absent, C₁-C₆-alkyl, C₂-C₆-alkenyl, —O—, —C(O)—, —N(R³)—;
X is C(R⁵) or N;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, C₁-C₆ alkylene-aryl, C₁-C₆ alkenylene-aryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸;
each R² and R⁷ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D}, NR^{B}C(O)R^{D}, or —C(O)NR^{B}R^{C};
each R³ is independently hydrogen, C₁-C₆-alkyl, C₁-C₆-haloalkyl, or cycloalkyl;
each of R^{4a} and R^{4b} is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R⁹; or
R^{4a} and R^{4b} are taken together with the nitrogen atom to which they are attached to form a heterocyclyl or heteroaryl, wherein the heterocyclyl and heteroaryl are optionally substituted with one or more R⁹.
R⁵ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D} NR^{B}C(O)R^{D}, or —C(O)NR^{B}R^{C};
R⁶ is C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D} NR^{B}C(O)R^{D}, or —C(O)NR^{B}R^{C};
each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R”;
each R⁹ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —NR^{B}R^{C}, —OR^{A}, —NR^{B}C(O)R^{D}, —C(O)NR^{B}R^{C}, —C(O)R^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹⁰;
each R¹⁰, R¹¹, and R¹²is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, halo, oxo, cyano, —OR^{A}, or —NR^{B}R^{C};
each R^{A} is independently hydrogen, C₁-C₆ alkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₁-C₆-heteroalkyl, cycloalkyl, heterocyclyl; or
R^{B} and R^{C} together with the atom to which they are attached form a 3-7-membered heterocyclyl ring optionally substituted with one or more R³;
each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
R¹³ is C₁-C₆-alkyl or halo;
m is 0, 1, 2, or 3;
n is 0, 1, or 2;
p and q are each independently 1, 2, 3, or 4;
o is 0, 1, 2, 3, 4, 5, 6, 7, 8, 9, 10, 11, 12, or 13; and
x is 0, 1, or 2.

2-3. (canceled)

4. The compound of claim 1, wherein A is a nitrogen-containing heteroaryl optionally substituted with one or more R¹.

5-6. (canceled)

7. The compound of claim 1, wherein A is selected from 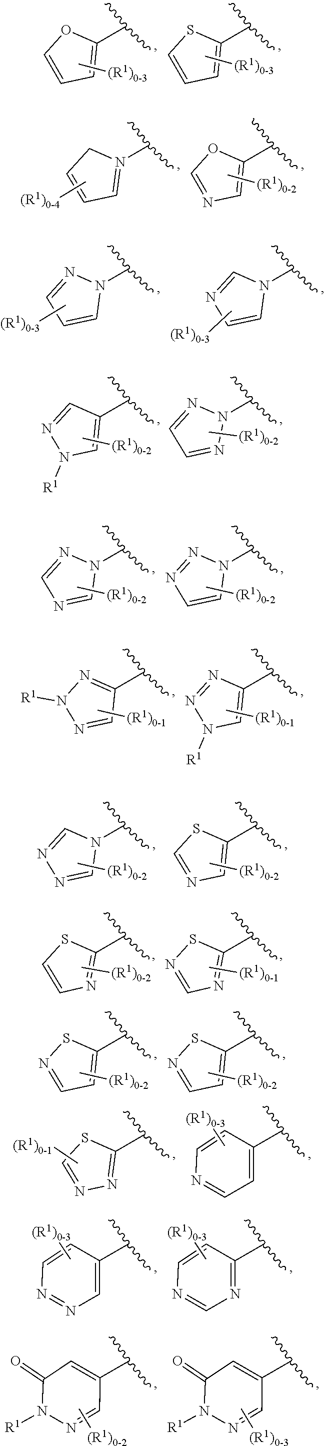 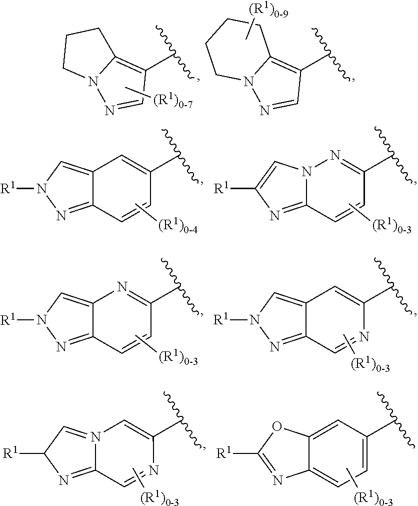
wherein R¹ is as described in claim 1.

8-9. (canceled)

10. The compound of claim 1, wherein A is selected from

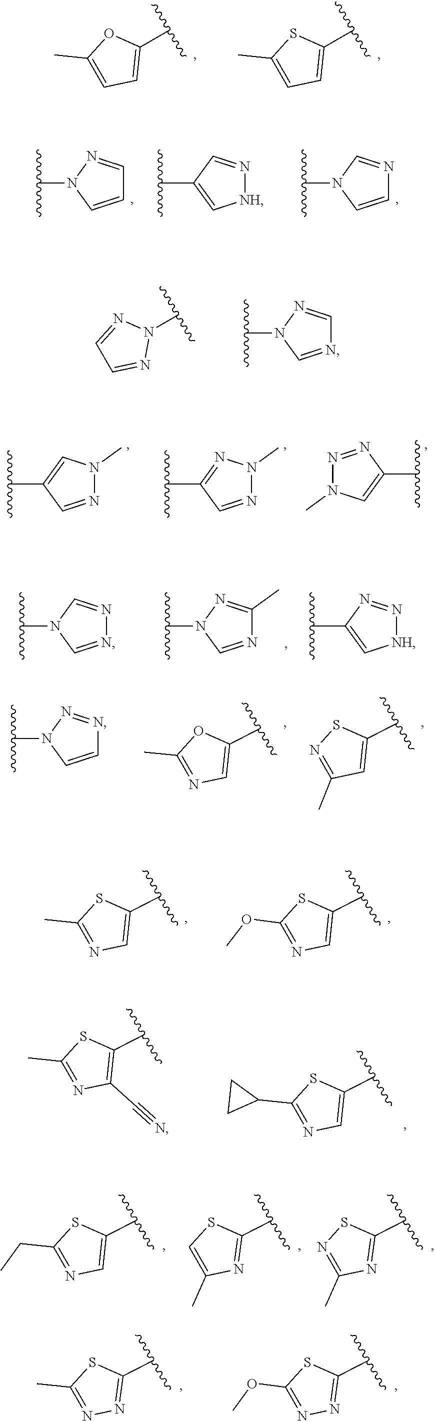

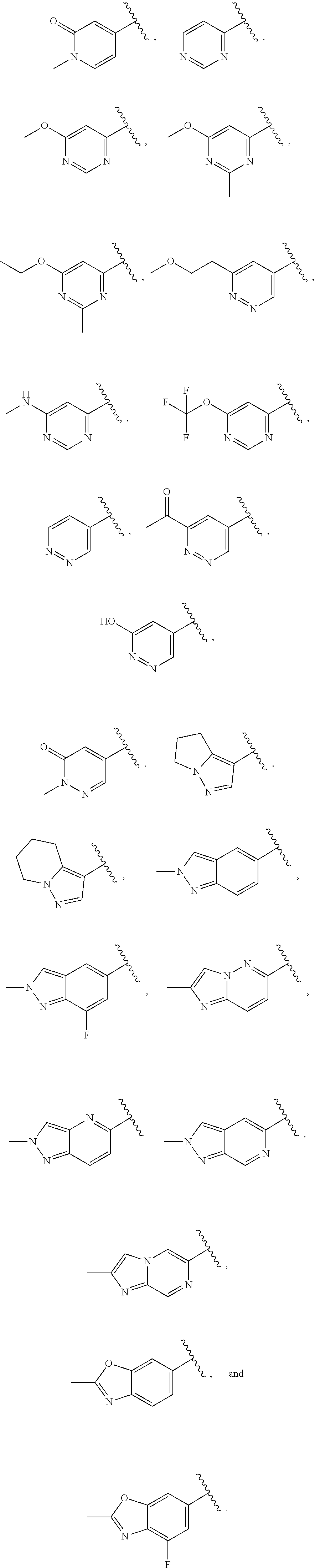

11-13. (canceled)

14. The compound of claim 1, wherein L is absent, —O—, or —N(R³)—.

15-19. (canceled)

20. The compound of claim 1, wherein X is N.

21-26. (canceled)

27. The compound of claim 1, wherein one of R^{4a} and R^{4b} is hydrogen or C₁-C₆-alkyl and the other of R^{4a} and R^{4b} is C₁-C₆-alkyl or cycloalkyl, each of which is optionally substituted with one or more R⁹.

28-30. (canceled)

31. The compound of claim 1, wherein 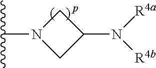
is selected from

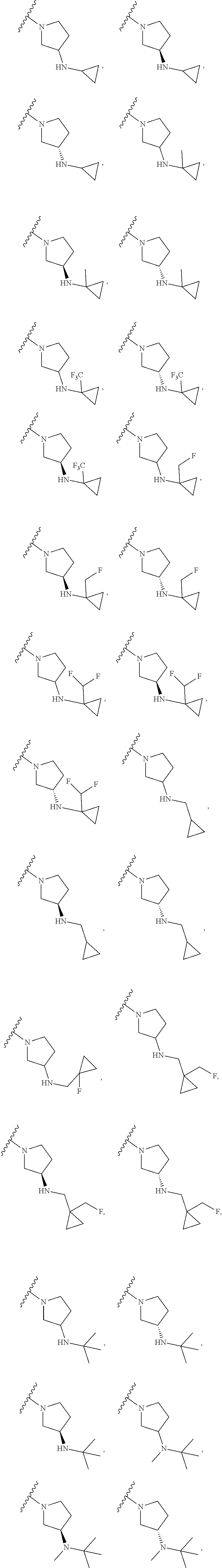

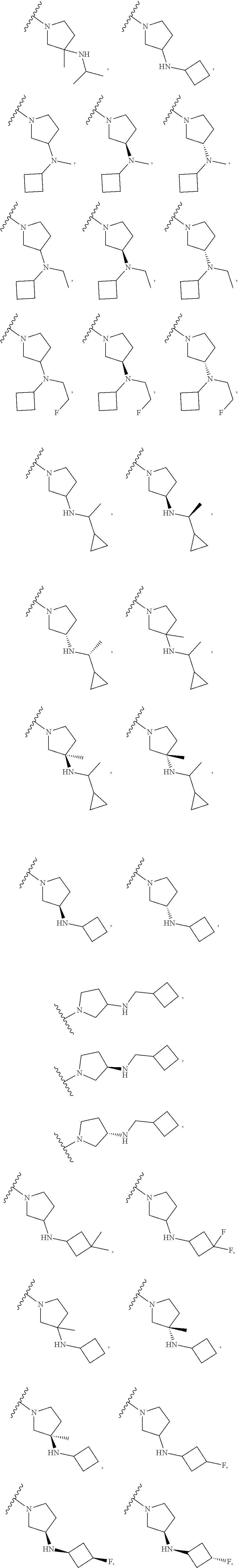

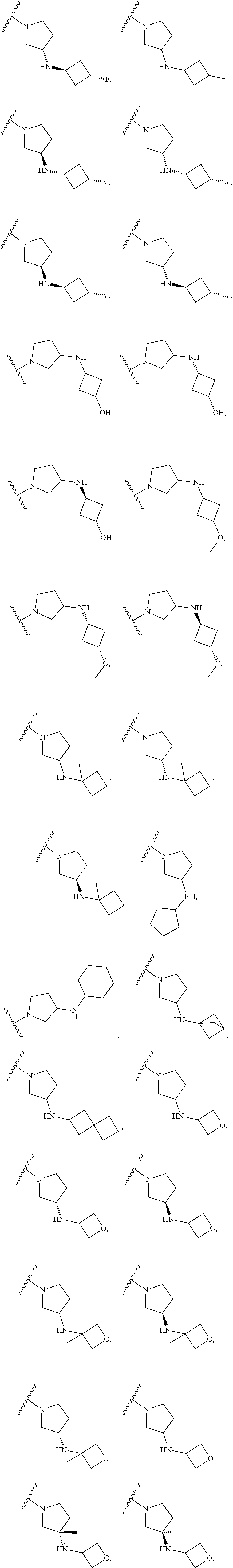

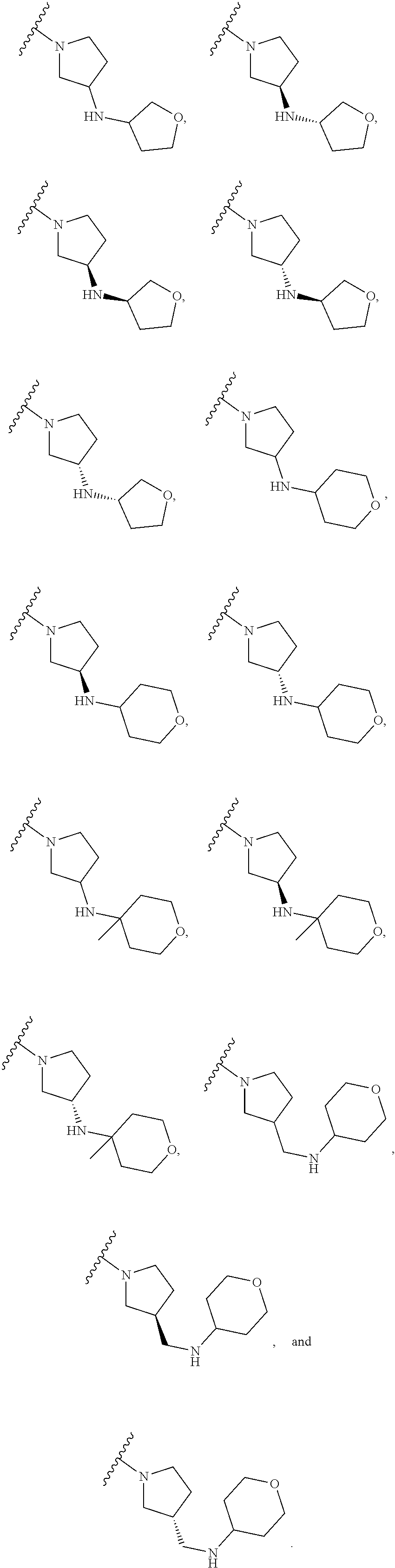

32. The compound of claim 1, wherein the compound is a compound of Formula (I-a): 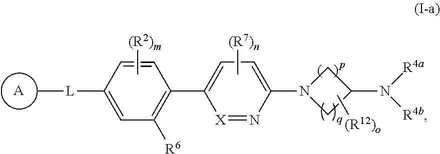
or a pharmaceutically acceptable salt, solvate, hydrate, tautomer, or stereoisomer thereof, wherein A, X, R², R^{4a}, R^{4b}, R⁷, R¹², m, n, p, o, and subvariables thereof are as described in claim 1, and R′ is hydrogen, C₁-C₆-alkyl or cycloalkyl.

33. (canceled)

34. The compound of claim 1, wherein the compound is a compound of Formula (I-c): 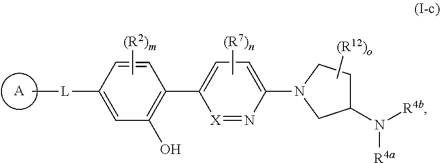
or a pharmaceutically acceptable salt, solvate, hydrate, tautomer, or stereoisomer thereof, wherein A, X, R², R^{4a}, R^{4b}, R⁷, R¹², m, n, o, and subvariables thereof are as described in claim 1.

35-39. (canceled)

40. The compound of claim 1, wherein the compound is selected from any one of the compounds shown in Table 1 or a pharmaceutically acceptable salt, solvate, hydrate, tautomer, or stereoisomer thereof.

41. A compound of Formula (II): 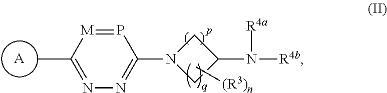
or a pharmaceutically acceptable salt, solvate, hydrate, tautomer, or stereoisomer thereof, wherein:
A is heteroaryl optionally substituted with one or more R¹;
M and P are each independently C(R²) or N;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A};
each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷;
each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or
R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or
each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶;
R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷;
each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or
R^{B} and R^{C} together with the nitrogen atom to which they are attached form a 3-7-membered heterocyclyl or heteroaryl, wherein each heterocyclyl or heteroaryl is optionally substituted with one or more R⁸.
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
R⁸ is C₁-C₆-alkyl, halo, or cycloalkyl;
n is 0, 1, 2, 3, 4, 5, 6, 7, 8, 9, 10, or 11;
p is 1, 2, 3, or 4;
q is 0, 1, 2, or 3; and
x is 0, 1, or 2.

42. The compound of claim 41, wherein A is a nitrogen-containing heteroaryl or nitrogen-containing heterocyclyl optionally substituted with one or more R¹.

43. (canceled)

44. The compound of claim 41, wherein A is selected from 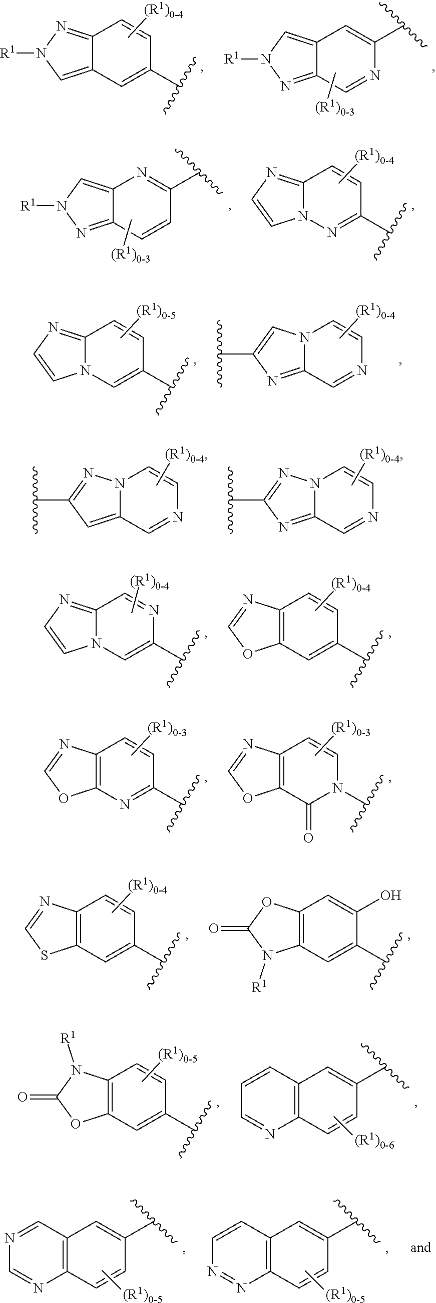 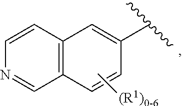
wherein R¹ is as described in claim 41.

45. (canceled)

46. The compound of claim 41, wherein A is selected from

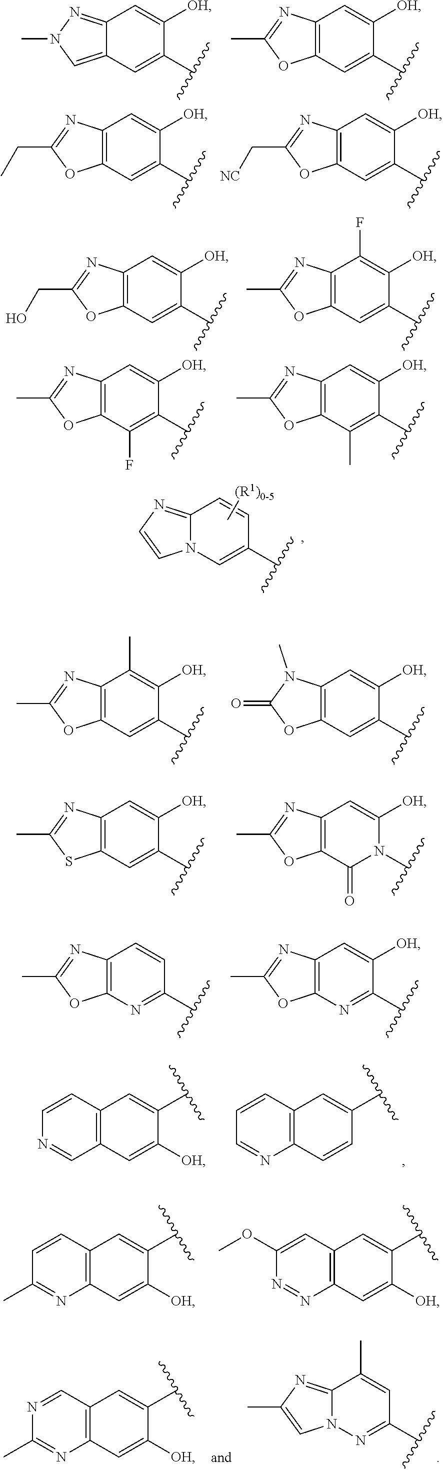

47. (canceled)

48. The compound of claim 41, wherein 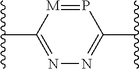
is selected from

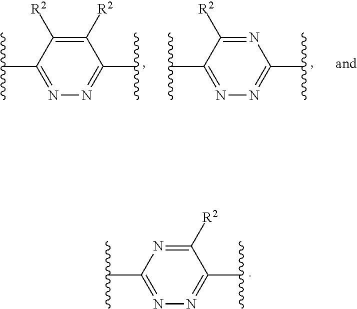

49-63. (canceled)

64. The compound of claim 41, wherein 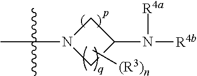
is selected from

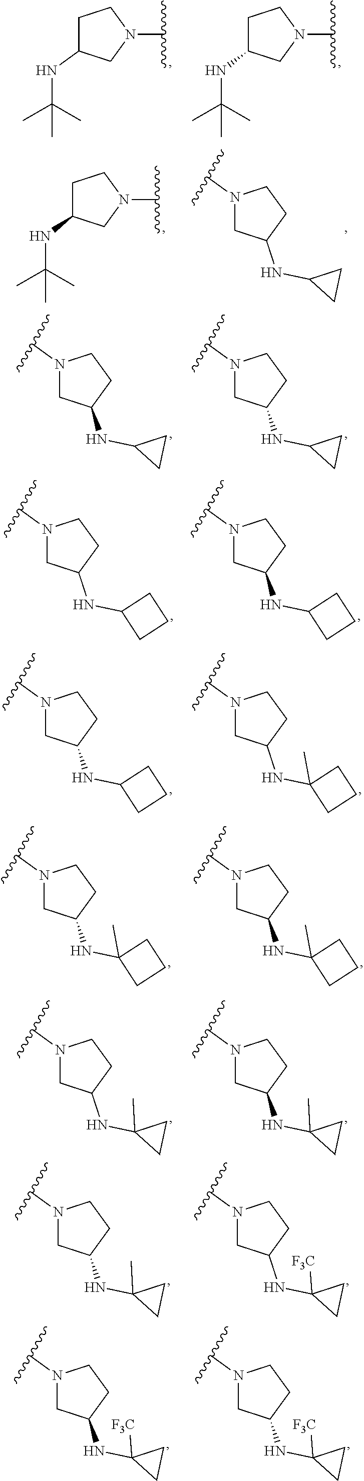

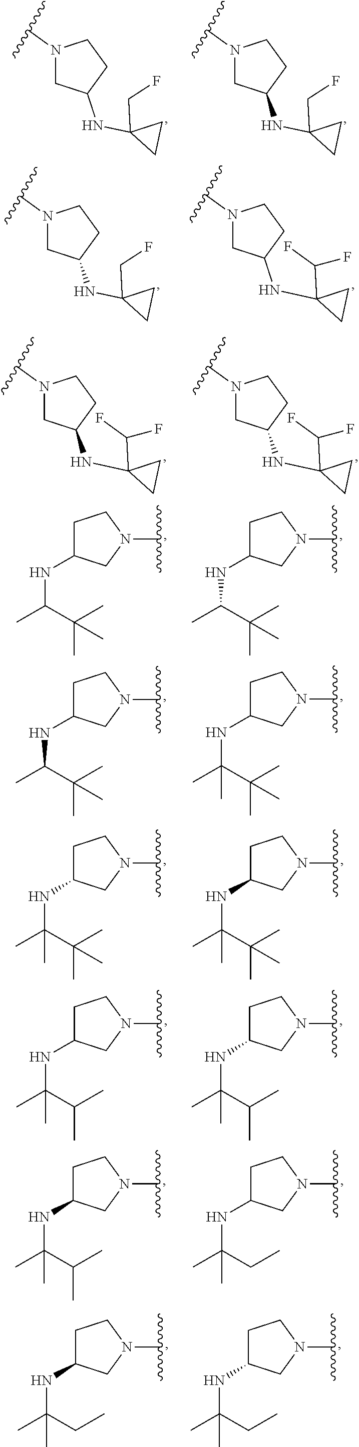

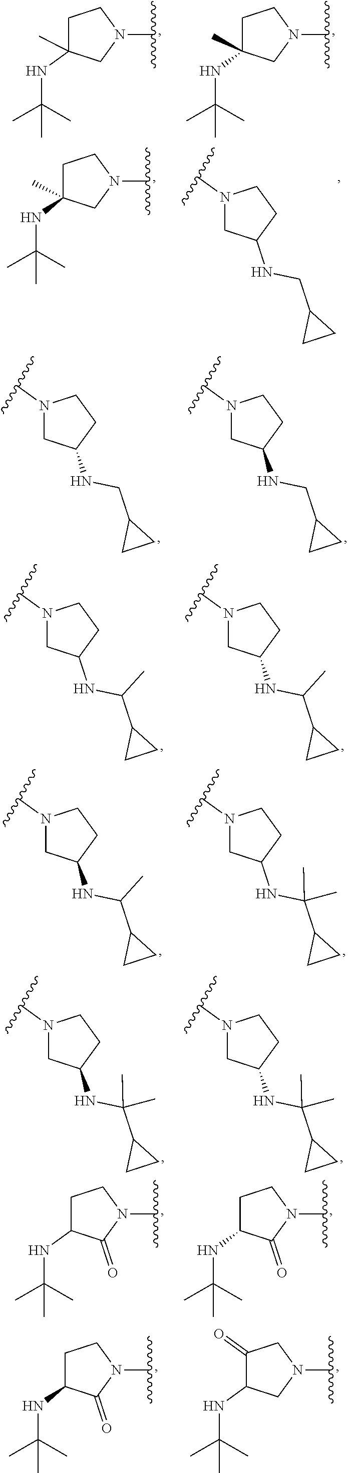

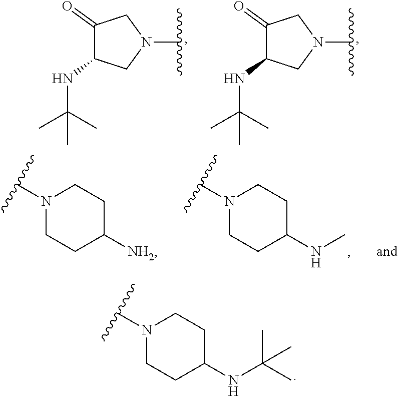

65-66. (canceled)

67. The compound of claim 41, wherein the compound is a compound of Formula (II-a): 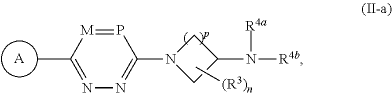
or a pharmaceutically acceptable salt, solvate, hydrate, tautomer, or stereoisomer thereof, wherein A, R³, R^{4a}, R^{4b}, n, p, and subvariables thereof are as described in claim 41.

68. (canceled)

69. The compound of claim 41, wherein the compound is a compound of Formula (II-c): 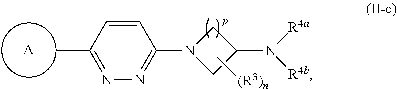
or a pharmaceutically acceptable salt, solvate, hydrate, tautomer, or stereoisomer thereof, wherein A, R³, R^{4a}, R^{4b}, n, p, and subvariables thereof are as described in claim 41.

70-73. (canceled)

74. A compound of Formula (III): 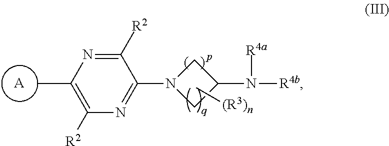
or a pharmaceutically acceptable salt, solvate, hydrate, tautomer, or stereoisomer thereof, wherein:
A is heteroaryl optionally substituted with one or more R¹;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A};
each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷;
each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or
R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or
each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶ R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷;
each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or
R^{B} and R^{C} together with the nitrogen atom to which they are attached form a 3-7-membered heterocyclyl or heteroaryl, wherein each heterocyclyl or heteroaryl is optionally substituted with one or more R⁸;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
R⁸ is C₁-C₆-alkyl, halo, or cycloalkyl;
n is 0, 1, 2, 3, 4, 5, 6, 7, 8, 9, 10, or 11;
p is 1, 2, 3, or 4;
q is 0, 1, 2, or 3; and
x is 0, 1, or 2.

75-96. (canceled)

97. A compound of Formula (IV): 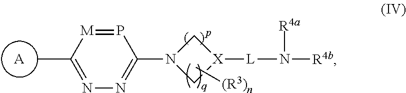
or a pharmaceutically acceptable salt, solvate, hydrate, tautomer, or stereoisomer thereof, wherein:
A is heteroaryl optionally substituted with one or more R¹;
M and P are each independently C(R²) or N;
X is C(R³) or N;
L is absent, C₁-C₆-alkylene, C₂-C₆-alkenylene, C₁-C₆-heteroalkylene, —C(O)—, —NR^{B}C(O)—, —C(O)NR^{B}—;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, or —OR^{A};
each R³ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, and haloalkyl is optionally substituted with one or more R⁷;
each of R^{4a} and R^{4b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, or heteroaryl, wherein each alkyl, alkenyl, alkynyl, heteroalkyl, haloalkyl, cycloalkyl, heterocyclyl, aryl, and heteroaryl is optionally substituted with one or more R⁶; or
R^{4a} and R^{4b} are taken together with the nitrogen atom to which they are attached to form a 3-7-membered heterocyclyl or heteroaryl, wherein each heterocyclyl and heteroaryl is optionally substituted with one or more R⁶; or
each of R^{4a} and R^{4b} is taken independently with the nitrogen atom to which it is attached to form a 3-7-membered spiro or fused heterocyclyl or heteroaryl with the adjacent heterocyclyl ring, wherein each heterocyclyl and heteroaryl is optionally substituted with one or more R⁶ R⁵ and R⁶ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷;
each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or
R^{B} and R^{C} together with the nitrogen atom to which they are attached form a 3-7-membered heterocyclyl or heteroaryl, wherein each heterocyclyl or heteroaryl is optionally substituted with one or more R⁸;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
R⁸ is C₁-C₆-alkyl, halo, or cycloalkyl;
n is 0, 1, 2, 3, 4, 5, 6, 7, 8, 9, 10, or 11;
p is 1, 2, 3, or 4;
q is 0, 1, 2, or 3; and
x is 0, 1, or 2.

98. The compound of claim 41, wherein the compound is selected from a compound provided in Table 2, or a pharmaceutically acceptable salt thereof.

99. A pharmaceutical composition comprising a compound of claim 1 and a pharmaceutically acceptable excipient.

100. The compound of claim 1, wherein the compound;
(i) alters a target nucleic acid;
(ii) binds to a target nucleic acid: or
(iii) stabilizes a target nucleic acid.

101-102. (canceled)

103. The compound of claim 1, wherein the compound;
i) increases splicing at splice site on a target nucleic acid (e.g., an RNA, e.g., a pre-mRNA), by about 0.5%1, %, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR; or
(ii) decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

104. (canceled)

105. A method of forming a complex comprising a component of a spliceosome (e.g., a major spliceosome component or a minor spliceosome component), a nucleic acid (e.g., a DNA, RNA, e.g., a pre-mRNA), and a compound of Formula (I), (II), (III), or (IV), according to claim 1, comprising contacting the nucleic acid (e.g., a DNA, RNA, e.g., a pre-mRNA) with a compound of Formula (I), (II), (III), (IV).

106. (canceled)

107. A method of altering the conformation of a nucleic acid (e.g., a DNA, RNA, e.g., a pre-mRNA) comprising contacting the nucleic acid with a compound of Formula (I), (II), (III), (IV), according to claim 1.

108. The method of claim 107, wherein the altering comprises;
(i) forming a bulge in the nucleic acid-;
(ii) stabilizing a bulge in the nucleic acid: or
(iii) reducing a bulge in the nucleic acid.

109-111. (canceled)

112. A method for treating a disease or disorder in a subject comprising administering to the subject a compound of Formula (I), (II), (III), (IV), according to claim 1.

113. The method of claim 112, wherein the disease or disorder comprises a proliferative disease; or a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

114-118. (canceled)

119. A composition for use in treating a disease or disorder in a subject comprising a compound of Formula (I), (II), (III), (IV), according to claim 1.

120. The composition for use of claim 119, wherein the disease or disorder comprises a proliferative disease; or a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

121-125. (canceled)
